(19) United States
(12) Patent Application Publication
(10) Pub. No.: US 2026/0347317 A1
(43) Pub. Date: Nov. 5, 2026

(54) SULFONIUM SALT, CHEMICALLY AMPLIFIED RESIST COMPOSITION, AND PATTERN FORMING METHOD

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/632,632

(22) Filed: Mar. 30, 2026

(30) Foreign Application Priority Data
Apr. 4, 2025 (JP) 2025-062666

(51) Int. Cl. C07C 381/12; G03F 7/004; G03F 7/039; G03F 7/20

(52) U.S. Cl. CPC C07C 381/12; G03F 7/0045; G03F 7/0397; G03F 7/2004

(57) ABSTRACT

The sulfonium salt can be used in a chemically amplified resist composition which has high sensitivity, is excellent in resolution, can improve lithographic performance such as LWR, CDU, EL, or DOF, and can suppress collapse of a resist pattern in photolithography using a high energy ray such as a far ultraviolet ray, an electron beam (EB), or EUV. The sulfonium salt has the following formula (1), wherein Z⁻ is an aromatic carboxylic acid anion which may have a substituent.

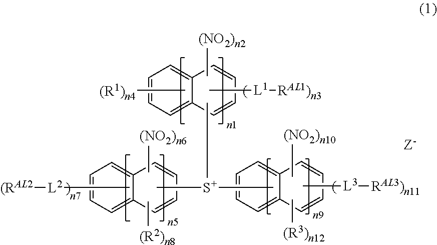

### CROSS-REFERENCE TO RELATED APPLICATION

This non-provisional application claims priority under 35 U.S.C. § 119(a) on Patent Application No. 2025-062666 filed in Japan on Apr. 4, 2025, the entire contents of which are hereby incorporated by reference.

### TECHNICAL FIELD

The present invention relates to a sulfonium salt, a chemically amplified resist composition, and a pattern forming method.

### BACKGROUND ART

With high integration and high speed of LSI, miniaturization of a pattern rule is rapidly progressing. This is because high-speed communication of 5G and artificial intelligence (Artificial intelligence (AI) have become widespread, and a high-performance device for processing them is required. As the most advanced miniaturization technique, mass production of 5 nm node devices by extreme ultraviolet (EUV) lithography having a wavelength of 13.5 nm is performed. Furthermore, studies using EUV lithography have been performed also in a next generation 3 nm node and a next generation 2 nm node device.

With a progress of miniaturization, image blurring due to acid diffusion has become a problem. In order to ensure resolution in a fine pattern after a 45 nm dimension size generation, it has been proposed that not only improvement of dissolution contrast that has been conventionally proposed but also control of acid diffusion is important (Non-Patent Document 1). However, since a chemically amplified resist composition increases sensitivity and contrast by acid diffusion, when a post exposure bake (PEB) temperature is lowered or a time is shortened to suppress acid diffusion to an utmost limit, the sensitivity and the contrast are significantly lowered.

A triangle trade-off relationship of sensitivity, resolution, and line width roughness (LWR) is indicated. In order to improve the resolution, it is necessary to suppress acid diffusion, but the sensitivity is lowered when an acid diffusion distance decreases.

It is effective to suppress acid diffusion by adding an acid generator that generates a bulky acid. Therefore, inclusion of a repeat unit derived from an onium salt having a polymerizable unsaturated bond in a polymer has been proposed. At this time, the polymer also functions as an acid generator (polymer-bound acid generator). Patent Document 1 proposes a sulfonium salt or an iodonium salt having a polymerizable unsaturated bond that generates a specific sulfonic acid. Patent Document 2 proposes a sulfonium salt in which a sulfonic acid is directly linked to a main chain.

In an acid-unstable group used in a (meth)acrylate polymer for an ArF resist composition, a deprotection reaction proceeds by using an acid generator that generates a sulfonic acid whose α-position is replaced with a fluorine atom, but the deprotection reaction does not proceed by using an acid generator that generates a sulfonic acid or a carboxylic acid whose α-position is not replaced with a fluorine atom. When a sulfonium salt or an iodonium salt that generates a sulfonic acid whose α-position is not replaced with a fluorine atom is mixed with a sulfonium salt or an iodonium salt that generates a sulfonic acid whose α-position is replaced with a fluorine atom, the sulfonium salt or the iodonium salt that generates a sulfonic acid whose α-position is not replaced with a fluorine atom undergoes ion exchange with the sulfonic acid whose α-position is replaced with a fluorine atom. Since the sulfonic acid whose α-position is replaced with a fluorine atom, generated by light is returned to a sulfonium salt or an iodonium salt by ion exchange, a sulfonium salt or an iodonium salt of a sulfonic acid or a carboxylic acid whose α-position is not replaced with a fluorine atom functions as a quencher. A resist composition using a sulfonium salt or an iodonium salt that generates a carboxylic acid as a quencher has been proposed (Patent Document 3).

A sulfonium salt type quencher that generates a carboxylic acid has been proposed. For example, sulfonium salts of salicylic acid and β-hydroxycarboxylic acid (Patent Document 4), a salicylic acid derivative (Patent Documents 5 and 6), fluorosalicylic acid (Patent Document 7), hydroxynaphthoic acid (Patent Document 8), salicylic acid having an iodine-containing aromatic substituent introduced therein (Patent Document 9), and salicylic acid having a cyclic acetal structure introduced therein (Patent Document 10) have been proposed. In particular, salicylic acid has a high effect of suppressing acid diffusion by an intramolecular hydrogen bond between a carboxy group and a hydroxy group.

It is pointed out that dimensional uniformity (CDU) of a resist pattern is reduced by aggregation of the quencher. It is expected to improve dimensional uniformity of a developed pattern by preventing aggregation of the quencher in a resist film and uniformizing distribution thereof.

In response to a demand for further miniaturization, there is a problem that swelling due to a developer occurs particularly at the time of alkali development in a positive resist, and pattern collapse occurs at the time of fine pattern formation. In order to respond to such a problem of miniaturization, it is important to develop a material for a novel resist composition, and it is desired to develop an onium salt type quencher which has favorable sensitivity, sufficiently controls acid diffusion, is excellent in solvent solubility, and is effective for suppressing pattern collapse.

### CITATION LIST

Patent Document 1: JP-A 2006-45311
Patent Document 2: JP-A 2006-178317
Patent Document 3: JP-A 2007-114431
Patent Document 4: WO 2018/159560
Patent Document 5: JP-A 2020-203984
Patent Document 6: JP-A 2020-91404
Patent Document 7: JP-A 2020-91312
Patent Document 8: JP-A 2019-120760
Patent Document 9: JP-A 2022-77505
Patent Document 10: WO 2023/189502
Non-Patent Document 1: SPIE Vol. 6520 65203L-1 (2007)

### SUMMARY OF THE INVENTION

In response to recent demand for high resolution of a resist pattern, a resist composition using a conventional sulfonium salt type acid generator and quencher cannot sufficiently suppress acid diffusion, and as a result, there is a problem that contrast or lithographic performance such as LWR, CDU, exposure latitude (EL), or focal depth (DOF) deteriorates. In addition, there is a problem that pattern collapse due to swelling occurs at the time of fine pattern formation.

The present invention has been made in view of the above circumstances, and an object thereof is to provide a sulfonium salt used in a chemically amplified resist composition which has high sensitivity, is excellent in resolution, can improve lithographic performance such as LWR, CDU, EL, or DOF, and can suppress collapse of a resist pattern in photolithography using a high energy ray such as a far ultraviolet ray, an electron beam (EB), or EUV, a chemically amplified resist composition containing the sulfonium salt as a quencher, and a pattern forming method using the chemically amplified resist composition.

As a result of intensive studies to achieve the above object, the present inventor has found that a sulfonium salt containing a sulfonium cation having a nitro group and an acid-unstable group having a tertiary/secondary ether structure, a tertiary/secondary ester structure, a tertiary/secondary carbonate structure, or an acetal structure in the same aromatic ring and an aromatic carboxylic acid anion is excellent in solvent solubility, and a chemically amplified resist composition using the sulfonium salt as a quencher has high sensitivity and high contrast, is excellent in lithographic performance such as LWR, CDU, EL, or DOF, is extremely effective for suppressing pattern collapse during fine pattern formation, and contributes also to suppression of defects during pattern development using an alkaline developer, and has completed the present invention.

That is, the present invention provides the following sulfonium salt, chemically amplified resist composition, and pattern forming method.

1. A sulfonium salt having the following formula (1):

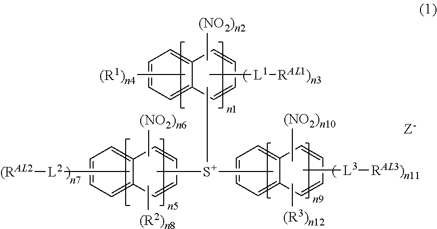

wherein n1 is 0 or 1, n2 is 0, 1, or 2, n3 is 0, 1, or 2, n4 is 0, 1, or 2, provided that 0≤n2+n3+n4≤5 when n1 is 0, and 0≤n2+n3+n4≤7 when n1 is 1, n5 is 0 or 1, n6 is 0, 1, or 2, n7 is 0, 1, or 2, n8 is 0, 1, or 2, provided that 0 s n6+n7+n8≤5 when n5 is 0, and 0≤n6+n7+n8≤7 when n5 is 1, n9 is 0 or 1, n10 is 0, 1, or 2, n11 is 0, 1, or 2, n12 is 0, 1, or 2, provided that 0≤n10+n11+n12≤5 when n9 is 0, and 0≤n10+n11+n12≤7 when n9 is 1, 1≤n2+n6+n10≤6, 1≤n3+n7+n11≤6,
R¹, R², and R³ are each independently a halogen atom, a cyano group, a hydroxy group, a carboxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n4 is 2, R¹s may be the same as or different from each other, and two R¹s may be bonded to each other to form a ring together with carbon atoms to which they are bonded, when n8 is 2, R²s may be the same as or different from each other, and two R²s may be bonded to each other to form a ring together with carbon atoms to which they are bonded, when n12 is 2, R³s may be the same as or different from each other, and two R³s may be bonded to each other to form a ring together with carbon atoms to which they are bonded,
L¹, L², and L³ are each independently an ether bond, an ester bond, or a carbonate bond, when n3 is 2, L's may be the same as or different from each other, when n7 is 2, L²s may be the same as or different from each other, when n11 is 2, L³s may be the same as or different from each other,
R^{AL1}, R^{AL2}, and R^{AL3} are each independently an acid-unstable group, when n3 is 2, R^{AL1}s may be the same as or different from each other, when n7 is 2, R^{AL2}s may be the same as or different from each other, when n11 is 2, R^{AL3}s may be the same as or different from each other,
two of the three aromatic rings bonded to S⁺ may be bonded to each other to form a ring together with the sulfur atom to which they are bonded, and
Z⁻ is an aromatic carboxylic acid anion which may have a substituent.

2. The sulfonium salt according to the item 1, having the following formula (1A):

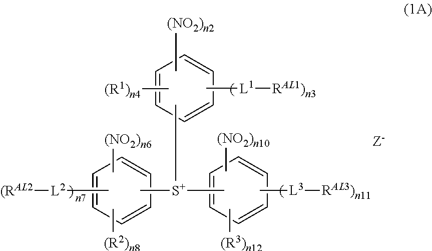

wherein n2 to n4, n6 to n8, n10 to n12, R¹ to R³, L¹ to L³, R^{AL1} to R^{AL3}, and Z⁻ are as defined above.

3. The sulfonium salt according to the item 1 or 2, wherein the acid-unstable group has the following formula (AL-1) or (AL-2):

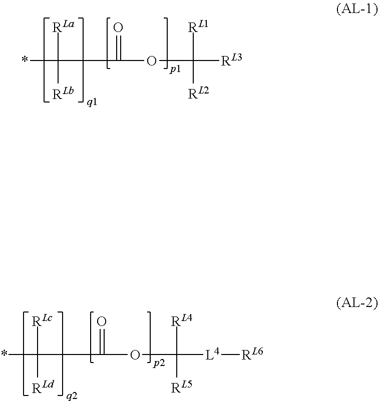

wherein p1 and p2 are each independently 0 or 1, q1 and q2 are each independently 0, 1, 2, 3, or 4,
R^{L1} and R^{L2} are each independently a C₁-C₁₂ hydrocarbyl group, R^{L3} is a hydrogen atom or a C₁-C₁₂ hydrocarbyl group, some of the (—CH₂-)s of the hydrocarbyl group of R^{L1}, R^{L2}, and R^{L3} may be replaced with —O— or —S—, when the hydrocarbyl group contains an aromatic ring, some or all of the hydrogen atoms of the aromatic ring may be replaced with a halogen atom, a cyano group, a nitro group, a C₁-C₄ alkyl group which may contain a halogen atom, or a C₁-C₄ alkoxy group which may contain a halogen atom, R^{L1} and R^{L2} may be bonded to each other to form a ring together with a carbon atom to which they are bonded, some of the (—CH₂-)s of the ring may be replaced with —O— or —S—, provided that when R^{L3} is a hydrogen atom, R^{L1} and R^{L2} are bonded to each other to form an alicyclic ring containing a multiple bond together with a carbon atom to which they are bonded, some of hydrogen atoms of the alicyclic ring may be replaced with a halogen atom,
R^{L4} and R^{L5} are each independently a hydrogen atom or a C₁-C₁₀ hydrocarbyl group, R^{L6} is a C₁-C₂₀ hydrocarbyl group, some of the (—CH₂-)s of the hydrocarbyl group may be replaced with —O— or —S—, R^{L5} and R^{L6} may be bonded to each other to form a C₃-C₂₀ heterocyclic group together with a carbon atom to which they are bonded and L⁴, some of the (—CH₂-)s of the heterocyclic group may be replaced with —O— or —S—,
L⁴ is —O— or —S—,
R^{La} to R^{Ld} are each independently a hydrogen atom or a C₁-C₂₀ hydrocarbyl group which may contain a heteroatom, and
* represents a bond with L¹, L², and L³.

4. The sulfonium salt according to any one of the items 1 to 3, wherein Z⁻ is an anion having the following formula (Z):

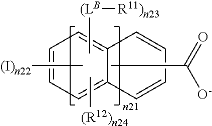

wherein n21 is 0 or 1, n22 is 0, 1, 2, 3, or 4, n23 is 0, 1, or 2, n24 is 0, 1, 2, 3, or 4, provided that 2≤n22+n23+n24≤5 when n21 is 0, provided that 2≤n22+n23+n24≤7 when n21 is 1,
L^{A} is a single bond, —O—, —S—, an ester bond, or a carbonate bond,
R¹¹ is a hydrogen atom or a C₁-C₂₀ hydrocarbyl group which may contain a heteroatom when L^{A} is a single bond, and is a hydrogen atom, a C₁-C₄₀ hydrocarbyl group which may contain a heteroatom (except for an acid-unstable group), or an acid-unstable group when L^{A} is —O—, —S—, an ester bond, or a carbonate bond,
R¹² is a halogen atom other than an iodine atom, a nitro group, a cyano group, a pentafluorosulfanyl group, a carboxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n24 is 2, 3, or 4, a plurality of R¹²s may be bonded to each other to form a ring structure together with carbon atoms on aromatic rings to which R¹²s are bonded, and some of the (—CH₂-)s of the rings may be replaced with —O— or —S—.

5. The sulfonium salt according to the item 4, wherein Z⁻ has the following formula (Z1):

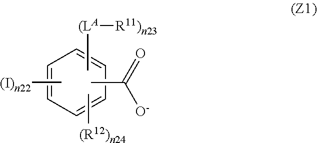

wherein n22 to n24, L^{A}, R¹¹, and R¹² are as defined above.

6. The sulfonium salt according to the item 4 or 5, wherein L^{A} is —O— or an ester bond.

7. The sulfonium salt according to the item 4 or 5, wherein L^{A} is —O—, —S—, an ester bond, or a carbonate bond, and R¹¹ is an acid-unstable group.

8. The sulfonium salt according to any one of the items 4 to 7, wherein the acid-unstable group of R¹¹ has the following formula (AL-3) or (AL-4):

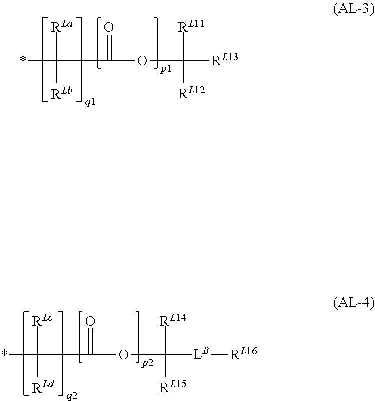

wherein p1 and p2 are each independently 0 or 1, q1 and q2 are each independently 0, 1, 2, 3, or 4,
R^{L11} and R^{L12} are each independently a C₁-C₁₂ hydrocarbyl group, R^{L13} is a hydrogen atom or a C₁-C₁₂ hydrocarbyl group, some of the (—CH₂-)s of the hydrocarbyl group of R^{L11}, R^{L12}, and R^{L13} may be replaced with —O— or —S—, when the hydrocarbyl group contains an aromatic ring, some or all of the hydrogen atoms of the aromatic ring may be replaced with a halogen atom, a cyano group, a nitro group, a C₁-C₄ alkyl group which may contain a halogen atom, or a C₁-C₄ alkoxy group which may contain a halogen atom, R^{L11} and R^{L12} may be bonded to each other to form a ring together with a carbon atom to which they are bonded, some of the (—CH₂-)s of the ring may be replaced with —O— or —S—, provided that when R^{L13} is a hydrogen atom, R^{L11} and R^{L12} are bonded to each other to form an alicyclic ring containing a multiple bond together with a carbon atom to which they are bonded, some of the hydrogen atoms of the alicyclic ring may be replaced with a halogen atom,
R^{L14} and R^{L15} are each independently a hydrogen atom or a C₁-C₁₀ hydrocarbyl group, R^{L16} is a C₁-C₂₀ hydrocarbyl group, some of the (—CH₂-)s of the hydrocarbyl group may be replaced with —O— or —S—, R^{L15} and R^{L16} may be bonded to each other to form a C₃-C₂₀ heterocyclic group together with a carbon atom to which they are bonded and L^{B}, some of the (—CH₂-)s of the heterocyclic group may be replaced with —O— or —S—,
L^{B} is —O— or —S—,
R^{La} to R^{Ld} are each independently a hydrogen atom or a C₁-C₂₀ hydrocarbyl group which may contain a heteroatom, and
* represents a bond with L^{A}.

9. A quencher formed of the sulfonium salt according to any one of the items 1 to 8.

10. A chemically amplified resist composition containing the quencher according to the item 9.

11. The chemically amplified resist composition according to the item 10, further containing a base polymer containing a polymer containing at least one selected from a repeat unit having the following formula (a1), a repeat unit having the following formula (a2), and a repeat unit having the following formula (a3):

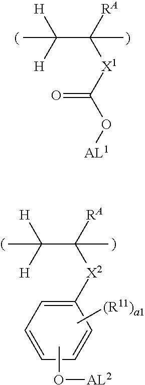

wherein R^{A}s are each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—N(H)—X¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
X² is a single bond, *—C(═O)—O—, or *—C(═O)—N(H)—X¹¹—,
* represents a bond with a carbon atom of a main chain,
R²¹ is a halogen atom, a cyano group, a hydroxy group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when a1 is 2, 3, or 4, R²¹s may be the same as or different from each other,
AL¹ and AL² are each independently an acid-unstable group, and a1 is 0, 1, 2, 3, or 4,

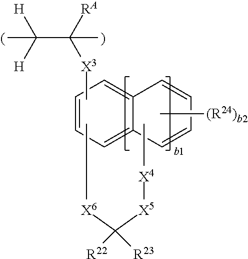

wherein b1 is 0 or 1, b2 is 0, 1, 2, or 3 when b1 is 0, and b2 is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—N(H)—, * represents a bond with a carbon atom of a main chain,
X⁴ is a single bond, a C₁-C₄ aliphatic hydrocarbylene group, a carbonyl group, a sulfonyl group, or a group obtained by combining these groups,
X⁵ and X⁶ are each independently an oxygen atom or a sulfur atom, provided that X⁴ and X⁶ are bonded to adjacent aromatic ring carbon atoms,
R²² and R²³ are each independently a hydrogen atom or a C₁-C₂₀ hydrocarbyl group which may contain a heteroatom, R²² and R²³ may be bonded to each other to form a ring together with a carbon atom to which they are bonded,
R²⁴ is a halogen atom, a hydroxy group, a cyano group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}), R^{24A} and R^{24B} are each independently a hydrogen atom or a C₁-C₆ hydrocarbyl group, when b2 is 2 or more, R²⁴s may be the same as or different from each other, and the plurality of R²⁴s may be bonded to each other to form a ring together with aromatic ring carbon atoms to which they are bonded.

12. The chemically amplified resist composition according to the item 11, wherein the polymer contains a repeat unit having the following formula (b1) or (b2):

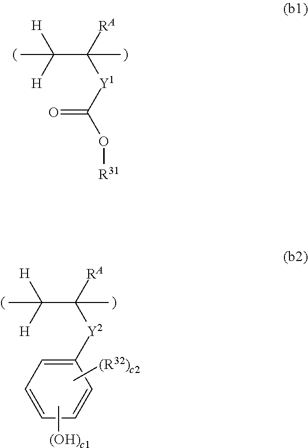

wherein R^{A}s are each independently a hydrogen atom, a fluorine atom, a methyl group, or a trifluoromethyl group,
Y¹ is a single bond, a phenylene group, a naphthylene group, *—C(═O)—O—Y¹¹—, or *—C(═O)—N(H)—Y¹¹—, the phenylene group or the naphthylene group may have a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom as a substituent, Y¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
Y² is a single bond or *—C(═O)—O—,
* represents a bond with a carbon atom of a main chain,
R³¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—),
R³² is a halogen atom, a carboxy group, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when c2 is 2, 3, or 4, R³²s may be the same as or different from each other,
c1 is 1, 2, 3, or 4, and c2 is 0, 1, 2, 3, or 4, provided that 1≤c1+c2≤5.

13. The chemically amplified resist composition according to the item 11 or 12, wherein the polymer contains at least one selected from a repeat unit having the following formula (c1), a repeat unit having the following formula (c2), a repeat unit having the following formula (c3), a repeat unit having the following formula (c4), and a repeat unit having the following formula (c5):

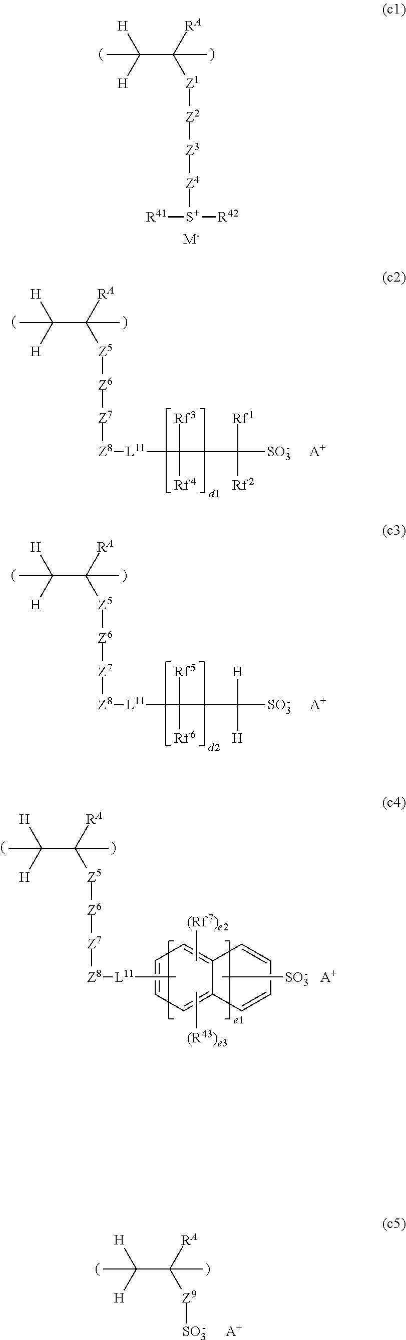

wherein d1 and d2 are each independently 0, 1, 2, or 3,
e1 is 0 or 1, e2 is 0, 1, 2, 3, or 4, e3 is 0, 1, 2, 3, or 4, provided that 0≤e2+e3≤4 when e1 is 0 and 0≤e2+e3≤6 when e1 is 1,
R^{A}s are each independently a hydrogen atom, a fluorine atom, a methyl group, or a trifluoromethyl group,
Z¹ is a single bond or a phenylene group which may have a substituent,
Z² is a single bond, **—C(═O)—O—Z²¹—, **—C(═O)N(H)—Z²¹—, or **—O—Z²¹—, Z²¹ is a C₁-C₆ aliphatic hydrocarbylene group, a phenylene group, or a divalent group obtained by combining these groups, and may contain a halogen atom, a carbonyl group, an ester bond, an ether bond, or a hydroxy group,
Z³ is a single bond, an ether bond, an ester bond, a sulfonate bond, an amide bond, a sulfonamide bond, a carbonate bond, or a carbamate bond,
Z⁴ is a single bond, or a C₁-C₆ aliphatic hydrocarbylene group, a phenylene group, or a divalent group obtained by combining these groups, and may contain a halogen atom, a carbonyl group, an ester bond, an ether bond, or a hydroxy group,
Z⁵s are each independently a single bond, a phenylene group which may contain a substituent, a naphthylene group which may contain a substituent, *—C(═O)—O—Z⁵¹—, or *—C(═O)—N(R)—Z⁵¹—, Z⁵¹ is a C₁-C₁₀ aliphatic hydrocarbylene group, a phenylene group, or a naphthylene group, and the aliphatic hydrocarbylene group may contain a halogen atom, a hydroxy group, an ether bond, an ester bond, or a lactone ring, R is a hydrogen atom or a C₁-C₁₀ hydrocarbyl group which may contain a heteroatom,
Z⁶ is a single bond, an ether bond, an ester bond, a sulfonate bond, an amide bond, a sulfonamide bond, a carbonate bond, or a carbamate bond,
Z⁷s are each independently a single bond, ***—Z⁷¹—C(═O)—O—, ***—C(═O)—N(R)—Z⁷¹—, or ***—O—Z⁷¹—, Z⁷¹ is a C₁-C₂₀ hydrocarbylene group which may contain a heteroatom, R is as defined above,
Z⁸s are each independently a single bond, ****—Z⁸¹—C(═O)—O—, ****—C(═O)—N(R)—Z⁸¹—, or ****—O—Z⁸¹—, Z⁸¹ is a C₁-C₂₀ hydrocarbylene group which may contain a heteroatom, R is as defined above,
Z⁹ is a single bond, a methylene group, an ethylene group, a phenylene group, a fluorinated phenylene group, a phenylene group replaced with a trifluoromethyl group, *—C(═O)—O—Z⁹¹—, *—C(═O)—N(R)—Z⁹¹—, or *—O—Z⁹¹—, Z⁹¹ is a C₁-C₆ aliphatic hydrocarbylene group, a phenylene group, a fluorinated phenylene group, or a phenylene group replaced with a trifluoromethyl group, and may contain a carbonyl group, an ester bond, an ether bond, or a hydroxy group, R is as defined above,
* represents a bond with a carbon atom of a main chain, ** represents a bond with Z¹, *** represents a bond with Z⁶, **** represents a bond with Z⁷,
L¹¹ is a single bond, an ether bond, an ester bond, a carbonyl group, a sulfonate bond, a sulfonamide bond, a carbonate bond, or a carbamate bond,
Rf¹ and Rf² are each independently a fluorine atom or a C₁-C₆ fluorinated saturated hydrocarbyl group,
Rf³ and Rf⁴ are each independently a hydrogen atom, a fluorine atom, or a C₁-C₆ fluorinated saturated hydrocarbyl group,
Rf⁵ and Rf⁶ are each independently a hydrogen atom, a fluorine atom, or a C₁-C₆ fluorinated saturated hydrocarbyl group, provided that not all Rf⁵s and Rf⁶s are simultaneously hydrogen atoms,
Rf⁷ is a fluorine atom, a C₁-C₆ fluorinated alkyl group, a C₁-C₆ fluorinated alkoxy group, a C₁-C₆ fluorinated alkylthio group, or a pentafluorosulfanyl group, when e2 is 2, 3, or 4, Rf⁷s may be the same as or different from each other,
R⁴¹ and R⁴² are each independently a C₁-C₂₀ hydrocarbyl group which may contain a heteroatom, R⁴¹ and R⁴² may be bonded to each other to form a ring together with a sulfur atom to which they are bonded,
R⁴³ is a halogen atom other than a fluorine atom, or a C₁-C₂₀ hydrocarbyl group which may contain a heteroatom, when e3 is 2, 3, or 4, R⁴³s may be the same as or different from each other, and a plurality of R⁴³s may be bonded to each other to form a ring together with carbon atoms to which they are bonded,
M⁻ is a non-nucleophilic counter ion, and
A⁺ is an onium cation.

14. The chemically amplified resist composition according to any one of the items 10 to 13, further containing an organic solvent.

15. The chemically amplified resist composition according to any one of the items 10 to 14, further containing a photoacid generator that generates a strong acid.

16. The chemically amplified resist composition according to any one of the items 10 to 15, further containing a quencher other than the quencher according to the item 9.

17. The chemically amplified resist composition according to any one of the items 10 to 16, further containing a surfactant.

18. A pattern forming method including: a step of forming a resist film on a substrate using the chemically amplified resist composition according to any one of the items 10 to 17; a step of exposing the resist film to a high energy ray; and a step of developing the exposed resist film using a developer.

19. The pattern forming method according to the item 18, wherein the high energy ray is a KrF excimer laser beam, an ArF excimer laser beam, EB, or EUV having a wavelength of 3 to 15 nm.

### Advantageous Effects of the Invention

When a pattern is formed using a chemically amplified resist composition containing the sulfonium salt of the present invention as a quencher, a resist pattern which has high contrast and favorable sensitivity, is excellent in lithographic performance such as EL, LWR, CDU, or DOF, and suppresses development defects can be formed.

### DESCRIPTION OF THE PREFERRED EMBODIMENT

### [Sulfonium Salt]

A sulfonium salt of the present invention has the following formula (1):

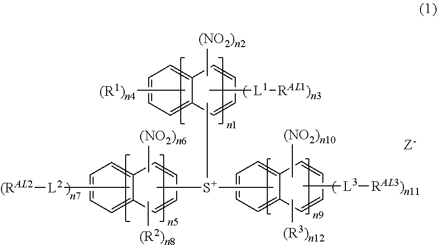

In the formula (1), n1 is 0 or 1. When n1 is 0, it is a benzene ring, and when n1 is 1, it is a naphthalene ring, but a benzene ring in which n1 is 0 is preferable from a viewpoint of solvent solubility. n2 is 0, 1, or 2. n3 is 0, 1, or 2. n4 is 0, 1, or 2. n4 is preferably 0 or 1 from a viewpoint of raw material procurement. Provided that 0≤n2+n3+n4≤5 when n1 is 0, and 0≤n2+n3+n4≤7 when n1 is 1.

In the formula (1), n5 is 0 or 1. When n5 is 0, it is a benzene ring, and when n5 is 1, it is a naphthalene ring, but a benzene ring in which n5 is 0 is preferable from a viewpoint of solvent solubility. n6 is 0, 1, or 2. n7 is 0, 1, or 2. n8 is 0, 1, or 2. n8 is preferably 0 or 1 from a viewpoint of raw material procurement. Provided that 0≤n6+n7+n8≤5 when n5 is 0, and 0≤n6+n7+n8≤7 when n5 is 1.

In the formula (1), n9 is 0 or 1. When n9 is 0, it is a benzene ring, and when n9 is 1, it is a naphthalene ring, but a benzene ring in which n9 is 0 is preferable from a viewpoint of solvent solubility. n10 is 0, 1, or 2. n11 is 0, 1, or 2. n12 is 0, 1, or 2. n12 is preferably 0 or 1 from a viewpoint of raw material procurement. Provided that 0≤n10+n11+n12≤5 when n9 is 0, and 0≤n10+n1+n12≤7 when n9 is 1.

In the formula (1), 1≤n2+n6+n10≤6, preferably 1≤n2+n6+n10≤3, and more preferably 1≤n2+n6+n10≤2. 1≤n3+n7+n11≤6, preferably 1≤n3+n7+n11≤3, and more preferably 1≤n3+n7+n11≤2.

In the formula (1), R¹, R², and R³ are each independently a halogen atom, a cyano group, a hydroxy group, a carboxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Examples of the halogen atom include a fluorine atom, a chlorine atom, a bromine atom, and an iodine atom. The hydrocarbyl group and hydrocarbyl moieties of the hydrocarbyloxy group and the hydrocarbylthio group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₂₀ alkenyl group such as a vinyl group, a 1-propenyl group, a 2-propenyl group, a butenyl group, or a hexenyl group; a C₃-C₂₀ cyclic unsaturated hydrocarbyl group such as a cyclohexenyl group; a C₆-C₂₀ aryl group such as a phenyl group or a naphthyl group; a C₇-C₂₀ aralkyl group such as a benzyl group, a 1-phenylethyl group, or a 2-phenylethyl group; and a group obtained by combining these groups. Among these, an aryl group is preferable. Some or all of the hydrogen atoms of the hydrocarbyl group may be replaced with a group containing a heteroatom such as an oxygen atom, a sulfur atom, a nitrogen atom, or a halogen atom, some of the (—CH₂-)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

When n4 is 2, R¹s may be the same as or different from each other. When n4 is 2, two R¹s may be bonded to each other to form a ring together with carbon atoms to which they are bonded. The ring is preferably a 5- to 8-membered ring. When n8 is 2, R²s may be the same as or different from each other. When n8 is 2, two R²s may be bonded to each other to form a ring together with carbon atoms to which they are bonded. The ring is preferably a 5- to 8-membered ring. When n12 is 2, R³s may be the same as or different from each other. When n12 is 2, two R³s may be bonded to each other to form a ring together with carbon atoms to which they are bonded. The ring is preferably a 5- to 8-membered ring.

In the formula (1), L¹, L², and L³ are each independently an ether bond, an ester bond, or a carbonate bond. Among these, L¹, L², and L³ are each independently preferably an ether bond or an ester bond. Among these, L¹, L², and L³ are each independently preferably an ether bond or an ester bond. When n3 is 2, L¹s may be the same as or different from each other. When n7 is 2, L²s may be the same as or different from each other. When n11 is 2, L³s may be the same as or different from each other.

In the formula (1), R^{AL1}, R^{AL2}, and R^{AL3} are each independently an acid-unstable group. When n3 is 2, R^{AL1}s may be the same as or different from each other. When n7 is 2, R^{AL2}s may be the same as or different from each other. When n11 is 2, R^{AL3}s may be the same as or different from each other.

The acid-unstable group preferably has the following formula (AL-1) or (AL-2):

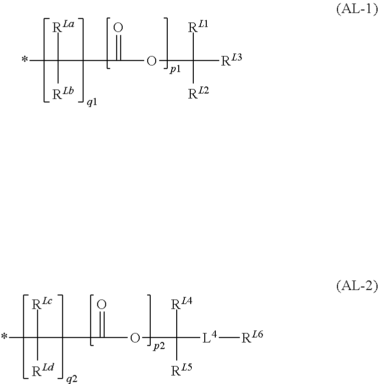

wherein * represents a bond with L¹, L², and L³.

In the formulas (AL-1) and (AL-2), p1 and p2 are each independently 0 or 1. q1 and q2 are each independently 0, 1, 2, 3, or 4. When p1 is 1, q1 is preferably 1 or 2, and when p1 is 0, q1 is 0. When p2 is 1, q2 is preferably 1 or 2, and when p2 is 0, q2 is 0.

In the formula (AL-1), R^{L1} and R^{L2} are each independently a C₁-C₁₂ hydrocarbyl group. R^{L3} is a hydrogen atom or a C₁-C₁₂ hydrocarbyl group. Some of the (—CH₂-)s of the hydrocarbyl group of R^{L1}, R^{L2}, and R^{L3} may be replaced with —O— or —S—, when the hydrocarbyl group contains an aromatic ring, some or all of the hydrogen atoms of the aromatic ring may be replaced with a halogen atom, a cyano group, a nitro group, a C₁-C₄ alkyl group which may contain a halogen atom, or a C₁-C₄ alkoxy group which may contain a halogen atom.

The C₁-C₁₂ hydrocarbyl groups of R^{L1}, R^{L2}, and R^{L3} may be saturated or unsaturated, and may be linear, branched, or cyclic. Specific examples thereof include: a C₁-C₁₂ alkyl group such as a methyl group, an ethyl group, a n-propyl group, an isopropyl group, a n-butyl group, an isobutyl group, a sec-butyl group, a tert-butyl group, a tert-pentyl group, a n-pentyl group, a n-hexyl group, a n-octyl group, a 2-ethylhexyl group, a n-nonyl group, a n-decyl group, a n-undecyl group, or a n-dodecyl group; a C₃-C₁₂ cyclic saturated hydrocarbyl group such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a norbornylmethyl group, an adamantyl group, an adamantylmethyl group, a tricyclo [5.2.1.0^{2,6}] decyl group, or a tetracyclo [6.2.1.1^{3,6}.0^{2,7}] dodecyl group; a C₂-C₁₂ alkenyl group such as a vinyl group, a 1-propenyl group, a 2-propenyl group, a butenyl group, a pentenyl group, or a hexenyl group; a C₂-C₁₂ alkynyl group such as an ethynyl group, a propynyl group, a butynyl group, a pentynyl group, or a hexynyl group; a C₃-C₁₂ cyclic unsaturated aliphatic hydrocarbyl group such as a cyclopentenyl group or a cyclohexenyl group; a C₆-C₁₂ aryl group such as a phenyl group, a naphthyl group, or an indanyl group; a C₇-C₁₂ aralkyl group such as a benzyl group, a 1-phenylethyl group, or a 2-phenylethyl group, and a group obtained by combining these groups.

R^{L1} and R^{L2} may be bonded to each other to form a ring together with a carbon atom to which they are bonded, and some of the (—CH₂-)s of the ring may be replaced with —O— or —S—. Specific examples of the ring formed at this time include a cyclopropane ring, a cyclobutane ring, a cyclopentane ring, a cyclohexane ring, a cycloheptane ring, a cyclooctane ring, a norbornane ring, an adamantane ring, a tricyclo [5.2.1.0^{2,6}] decane ring, and a tetracyclo [6.2.1.1^{3,6}.0^{2,7}] dodecane ring. Some of the (—CH₂-)s of the ring may be replaced with —O— or —S—. Provided that when R^{L3} is a hydrogen atom, R^{L1} and R^{L2} are bonded to each other to form an alicyclic ring containing a multiple bond together with a carbon atom to which they are bonded. Some of hydrogen atoms of the alicyclic ring may be replaced with a halogen atom.

In the formula (AL-2), R^{L4} and R^{L5} are each independently a hydrogen atom or a C₁-C₁₀ hydrocarbyl group. The C₁-C₁₀ hydrocarbyl groups of R^{L4} and R^{L5} may be saturated or unsaturated, and may be linear, branched, or cyclic. Specific examples thereof include those similar to those having 1 to 10 carbon atoms among those exemplified as the hydrocarbyl group of R^{L1}, R^{L2}, and R^{L3}.

In the formula (AL-2), R^{L6} is a C₁-C₂₀ hydrocarbyl group, and some of the (—CH₂-)s of the hydrocarbyl group may be replaced with —O— or —S—. The hydrocarbyl group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, a norbornylmethyl group, an adamantyl group, an adamantylmethyl group, a tricyclo [5.2.1.0^{2,6}] decyl group, or a tetracyclo [6.2.1.1^{3,6}.0^{2,7}] dodecyl group; a C₂-C₂₀ alkenyl group such as a vinyl group, a propenyl group, a butenyl group, a pentenyl group, or a hexenyl group; a C₂-C₂₀ alkynyl group such as an ethynyl group, a propynyl group, a butynyl group, a pentynyl group, or a hexynyl group; a C₃-C₂₀ cyclic unsaturated aliphatic hydrocarbyl group such as a cyclopentenyl group, a cyclohexenyl group, or a norbornenyl group; a C₆-C₂₀ aryl group such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, or a tert-butylnaphthyl group; a C₇-C₂₀ aralkyl group such as a benzyl group or a phenethyl group; and a group obtained by combining these groups. R^{L5} and R^{L6} may be bonded to each other to form a C₃-C₂₀ heterocyclic group together with a carbon atom and L⁴ to which they are bonded, and some of the (—CH₂-)s of the heterocyclic group may be replaced with —O— or —S—.

In the formula (AL-2), L⁴ is —O— or —S—.

In the formulas (AL-1) and (AL-2), R^{La} to R^{Ld} are each independently a hydrogen atom or a C₁-C₂₀ hydrocarbyl group which may contain a heteroatom. The C₁-C₂₀ hydrocarbyl group may be saturated or unsaturated, and may be linear, branched, or cyclic. Specific examples thereof include, but are not limited to, those similar to those exemplified as the hydrocarbyl group of R^{L6}.

Specific examples of the acid-unstable group having the formula (AL-1) include, but are not limited to, those listed below. Note that, in the following formulas, * represents a bond with L¹, L², and L³.

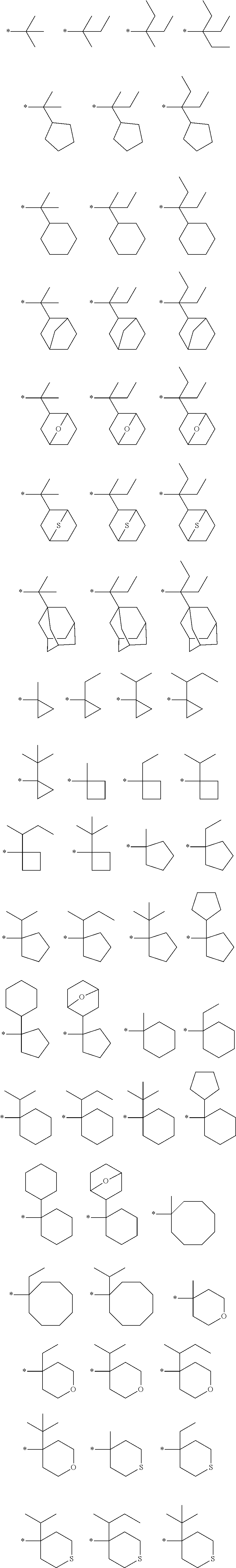

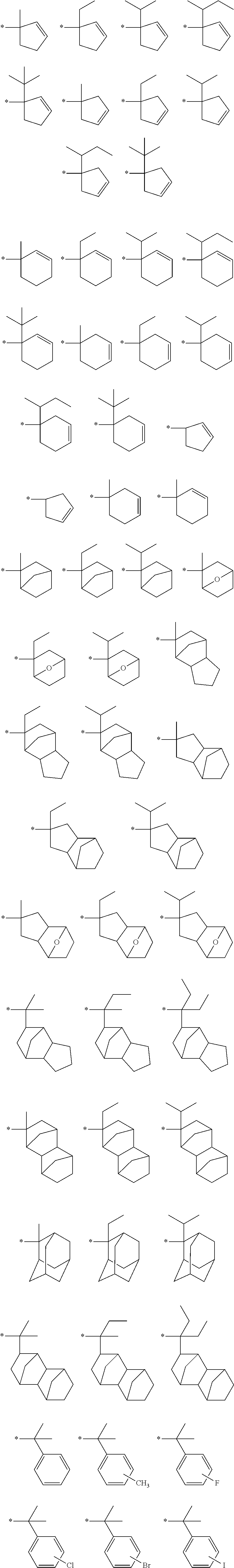

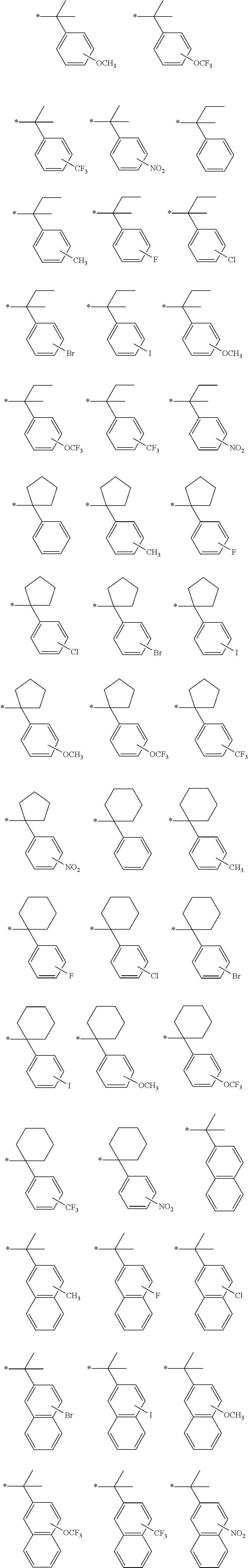

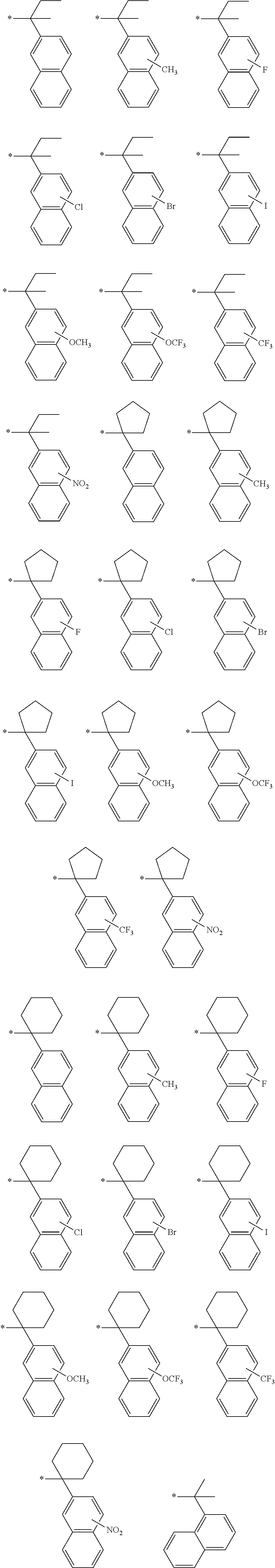

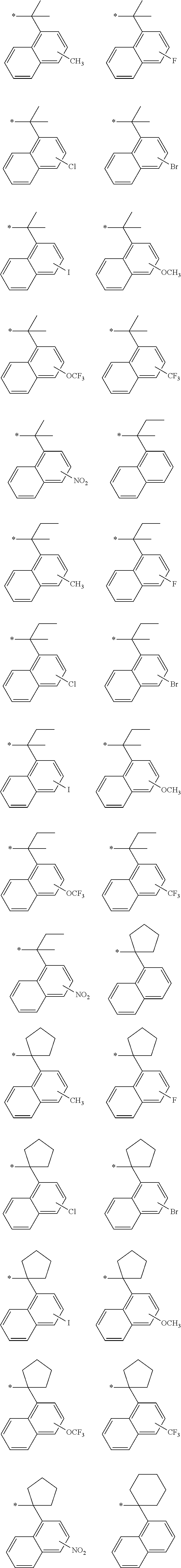

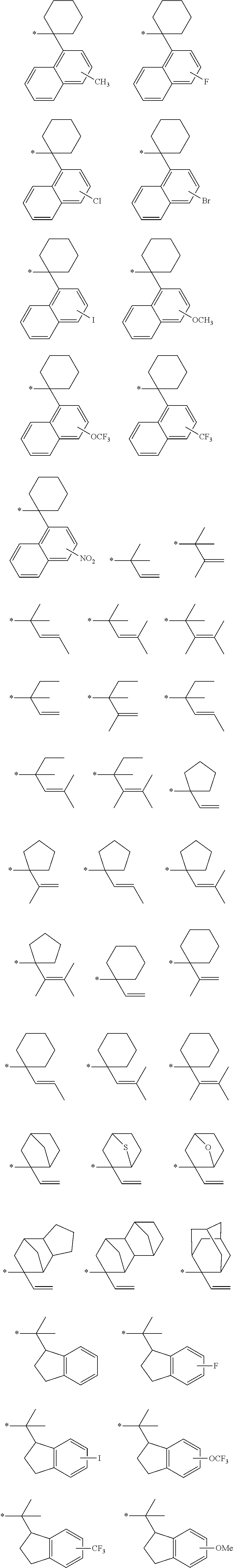

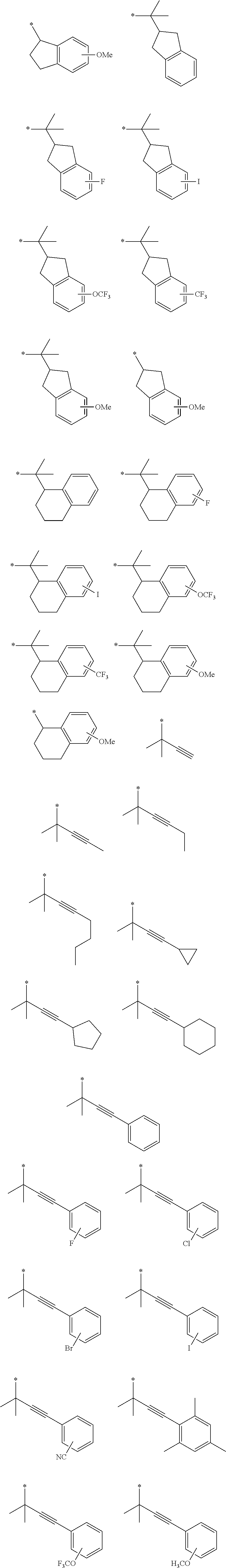

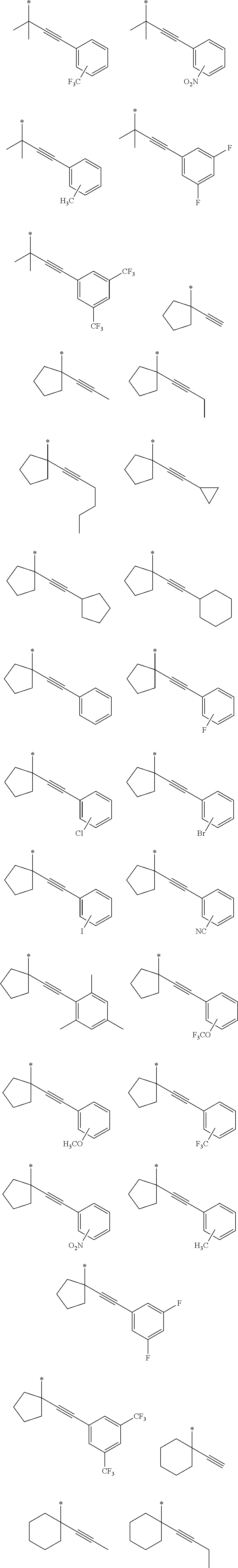

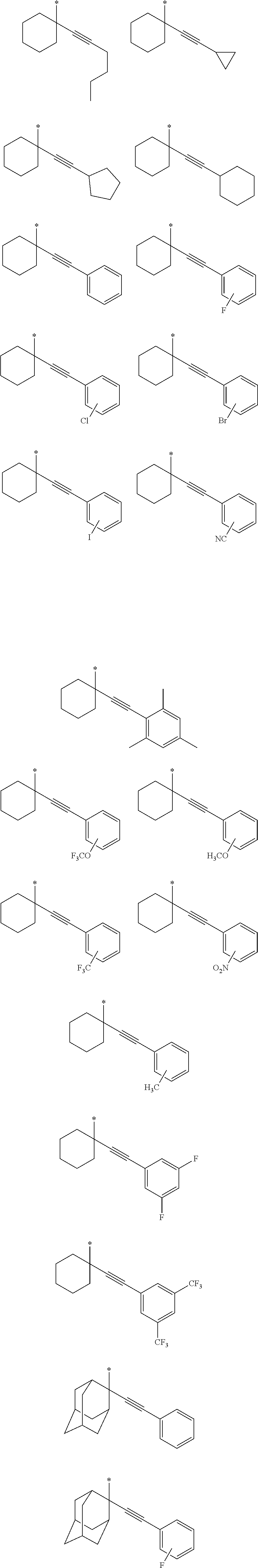

Specific examples of the acid-unstable group having the formula (AL-2) include, but are not limited to, those listed below. Note that, in the following formulas, * represents a bond with L¹, L², and L³.

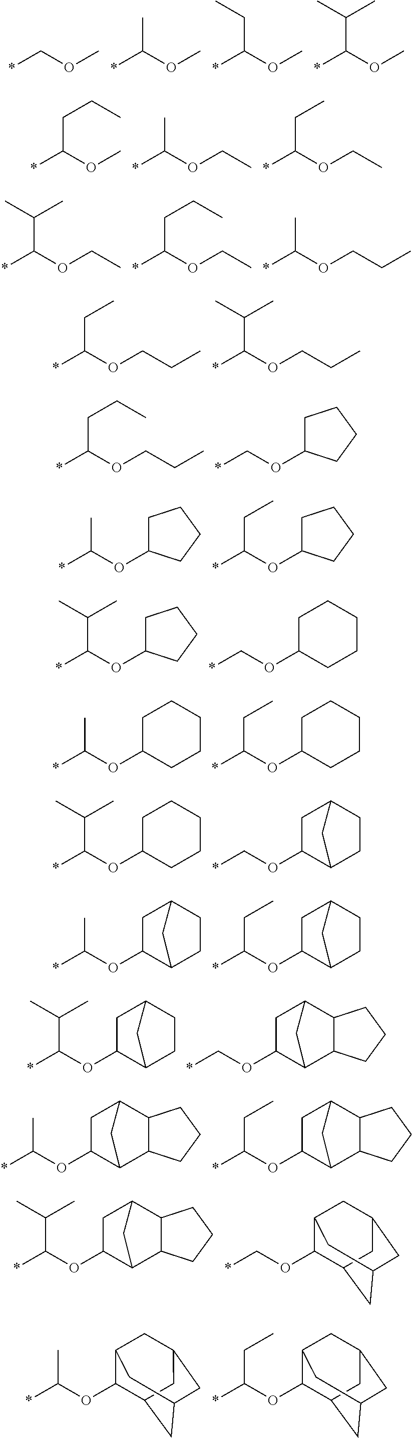

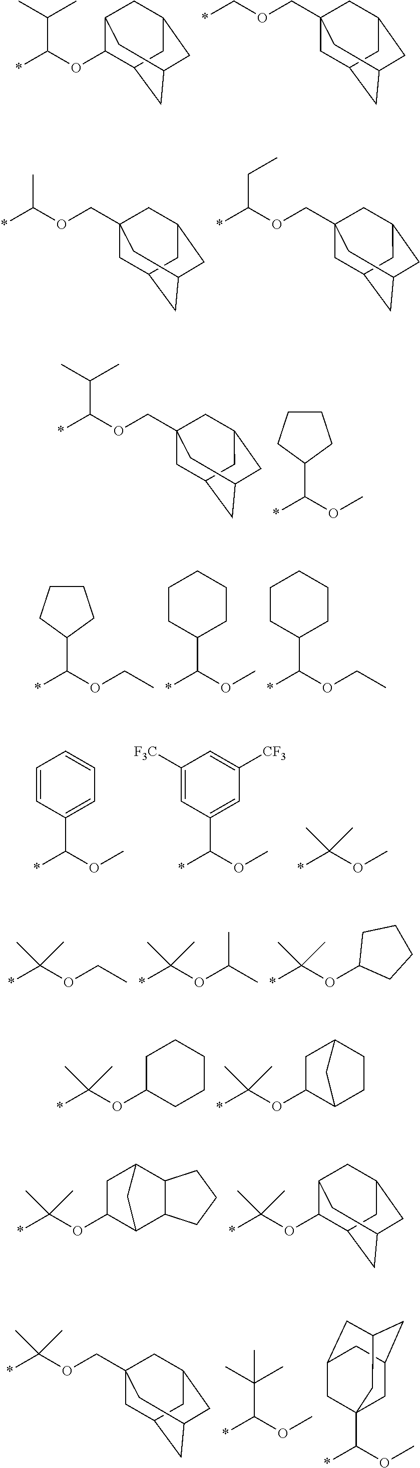

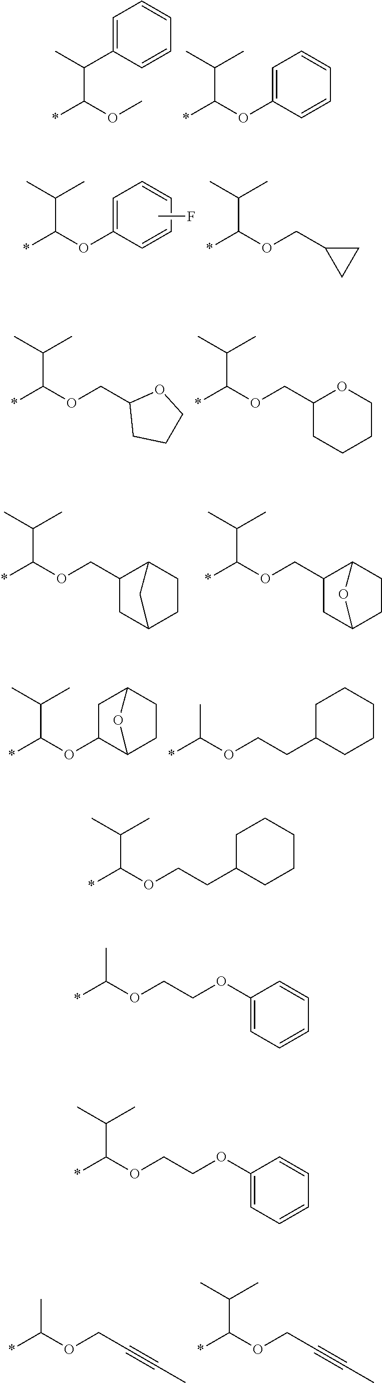

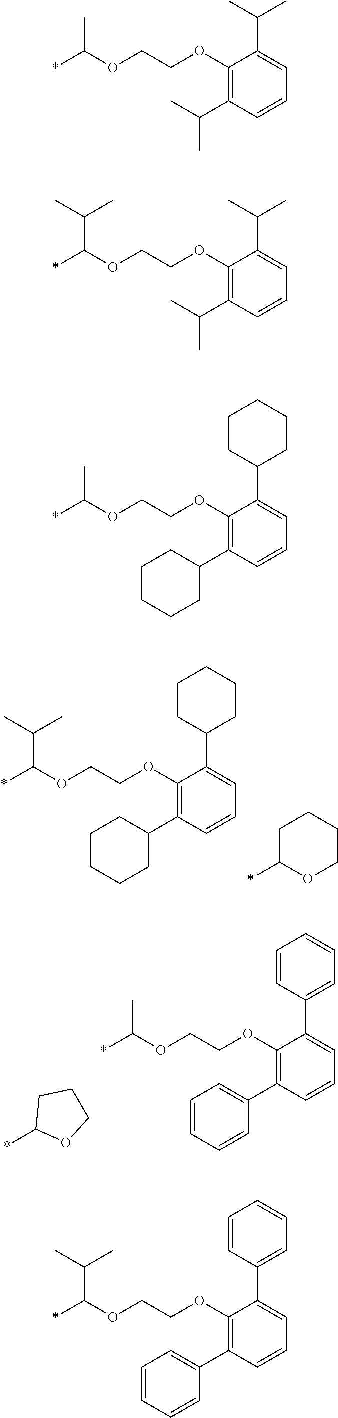

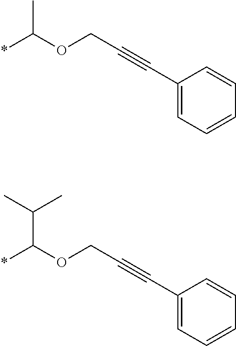 [SMILES: *C(C)OCC#Cc1ccccc1 ; *C(OCC#Cc1ccccc1)C(C)C]

In the formula (1), the nitro group and -L¹-R^{AL1} are preferably bonded to carbon atoms adjacent to each other. By the nitro group and -L¹-R^{AL1} being adjacent to each other, acidity of a phenol or a carboxylic acid generated by deprotecting —R^{AL1} is improved due to an electron withdrawing property of the nitro group, and dissolution contrast is improved.

In the formula (1), the nitro group and -L²-R^{AL2} are preferably bonded to carbon atoms adjacent to each other. By the nitro group and -L²-R^{AL2} being adjacent to each other, acidity of a phenol or a carboxylic acid generated by deprotecting —R^{AL2} is improved due to an electron withdrawing property of the nitro group, and dissolution contrast is improved.

In the formula (1), the nitro group and -L³-R^{AL3} are preferably bonded to carbon atoms adjacent to each other. By the nitro group and -L³-R^{AL3} being adjacent to each other, acidity of a phenol or a carboxylic acid generated by deprotecting —R^{AL3} is improved due to an electron withdrawing property of the nitro group, and dissolution contrast is improved.

Two of the three aromatic rings bonded to S⁺ may be bonded to each other to form a ring together with the sulfur atom to which they are bonded. At this time, specific examples of the structure of the ring include those having the following formulas:

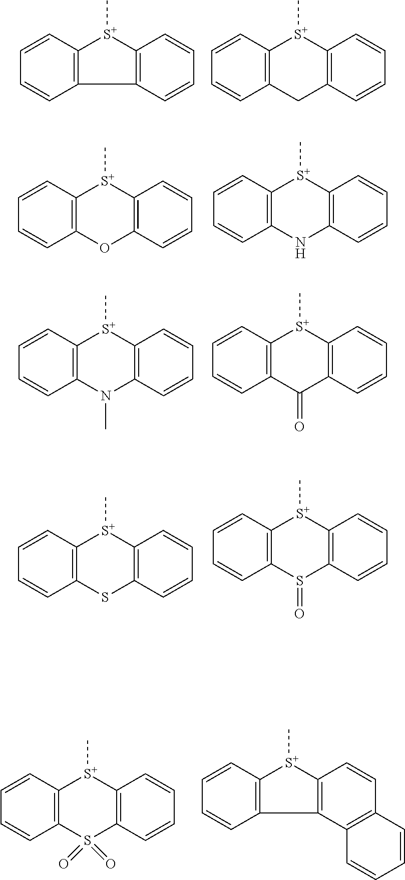

wherein the dashed line represents a bond.

The sulfonium salt having the formula (1) preferably has the following formula (1A):

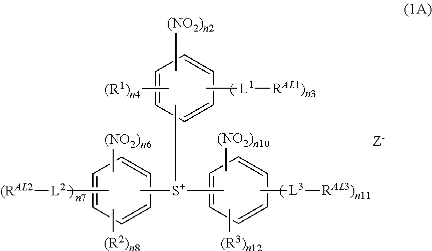

wherein n2 to n4, n6 to n8, n10 to n12, R¹ to R³, L¹ to L³, and R^{AL1} to R^{AL3} are as defined above. Z⁻ is described below.

Specific examples of the cation of the sulfonium salt having the formula (1) include, but are not limited to, those listed below. Note that, in the following formulas, Me is a methyl group.

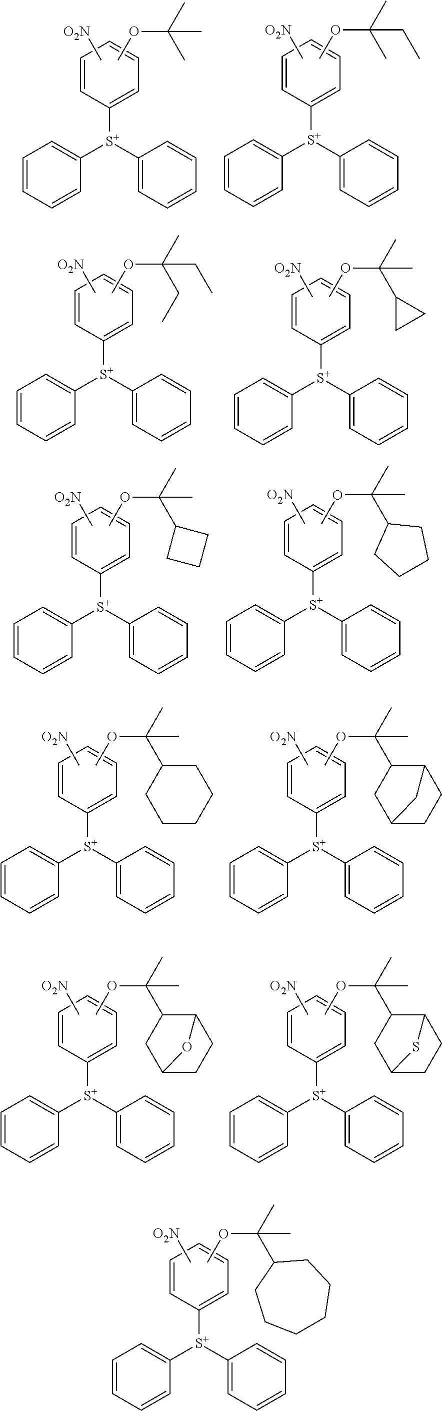

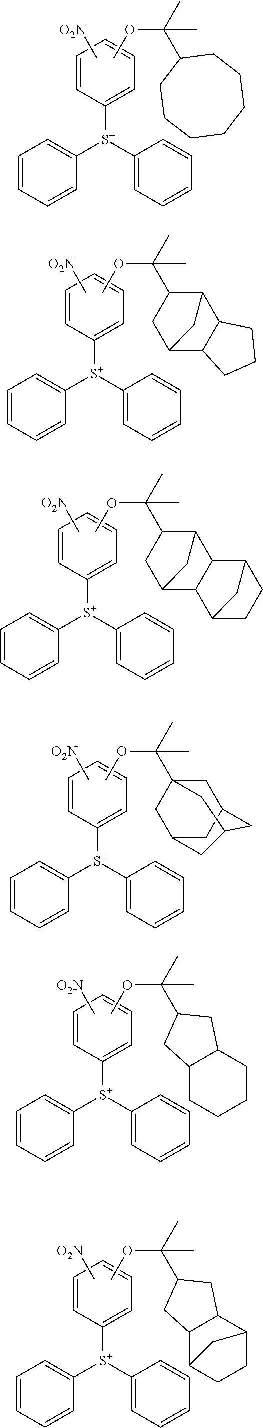

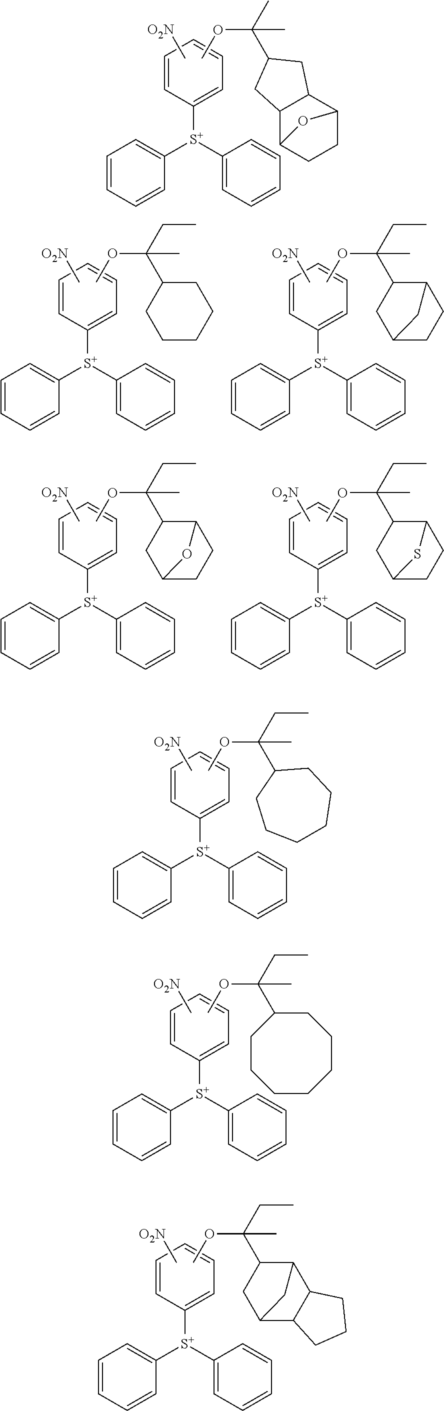

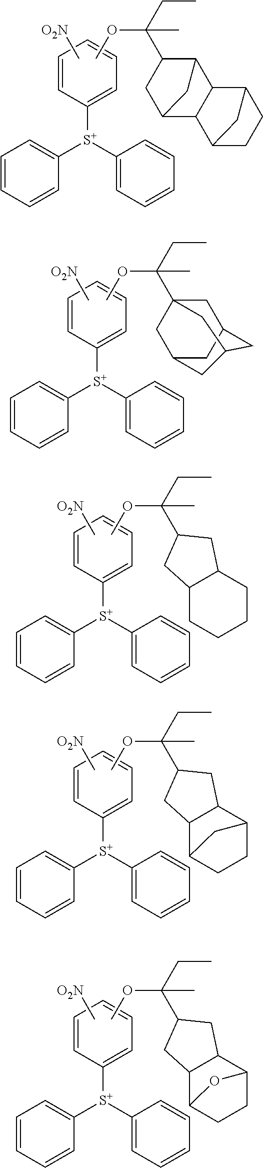

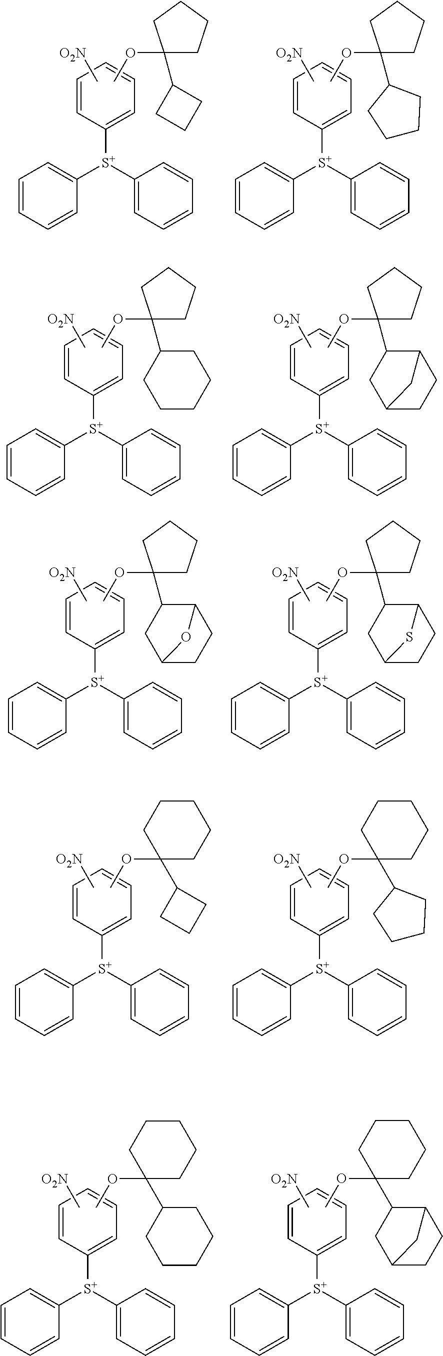

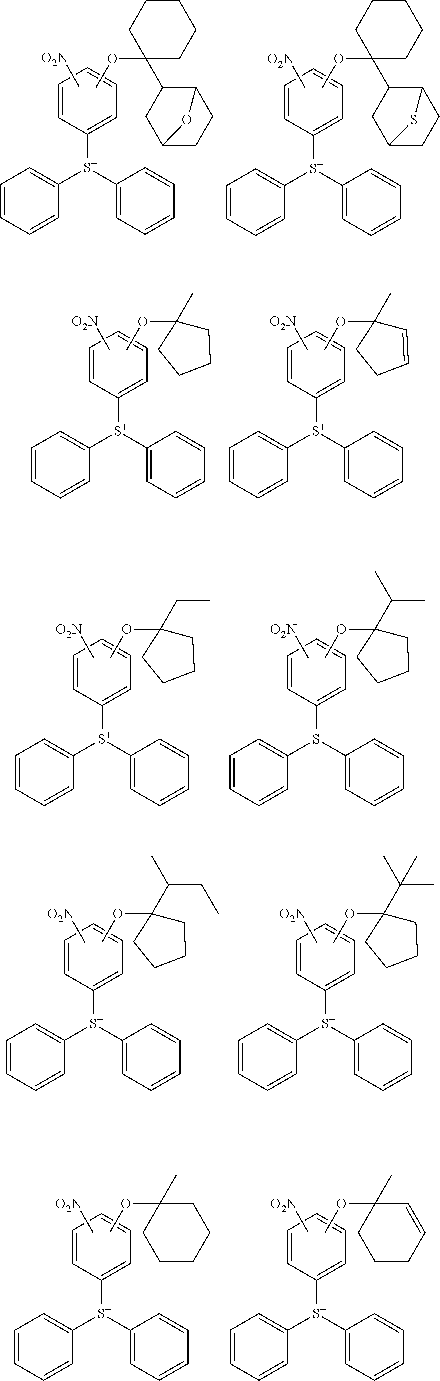

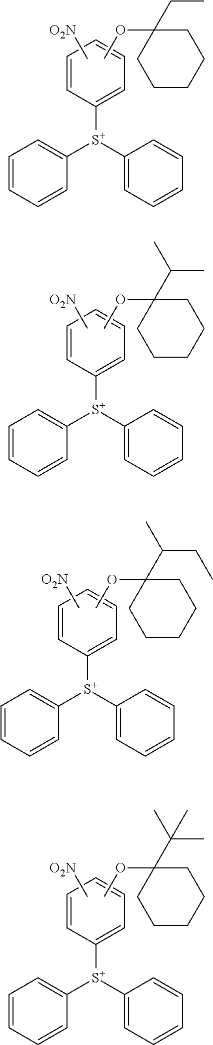

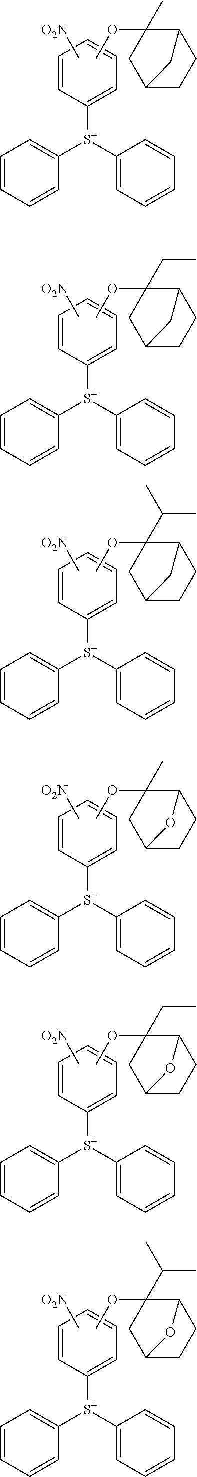

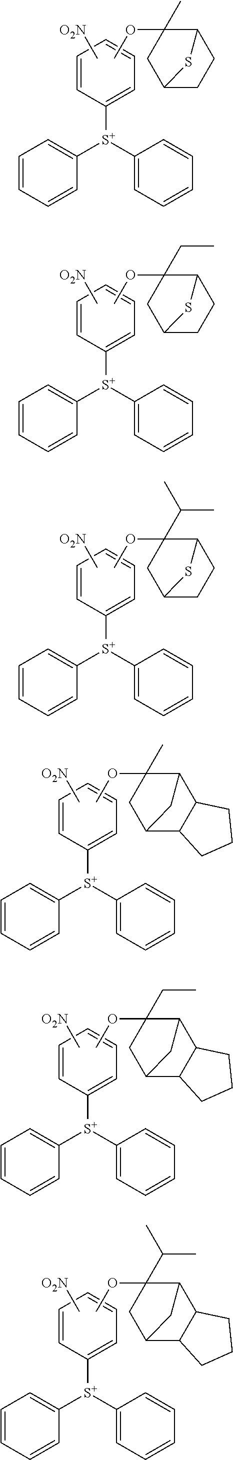

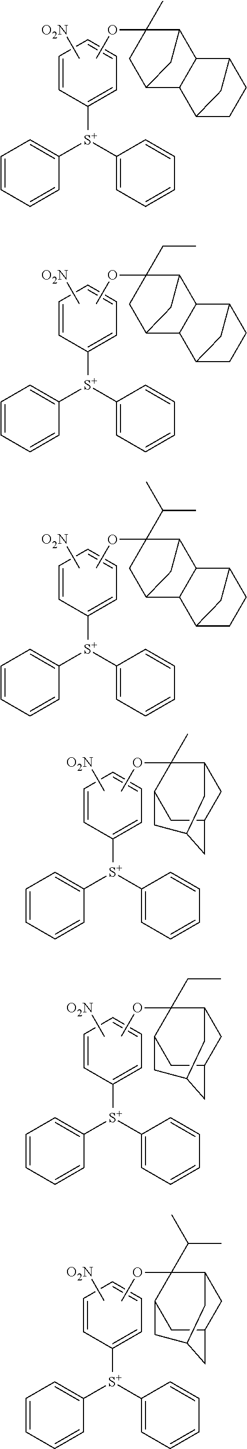

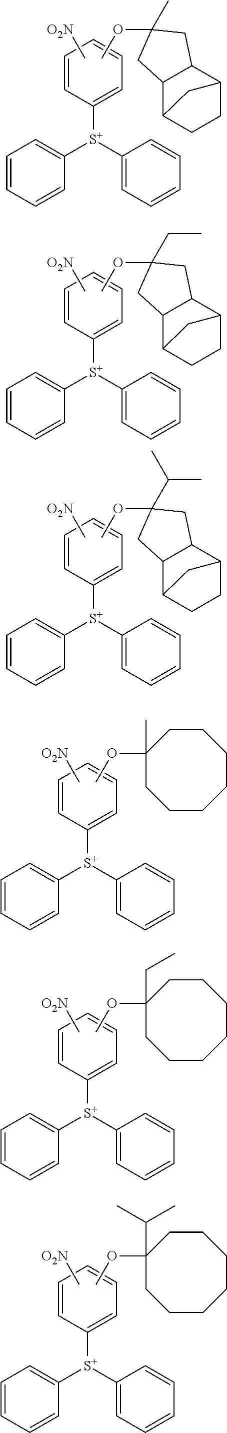

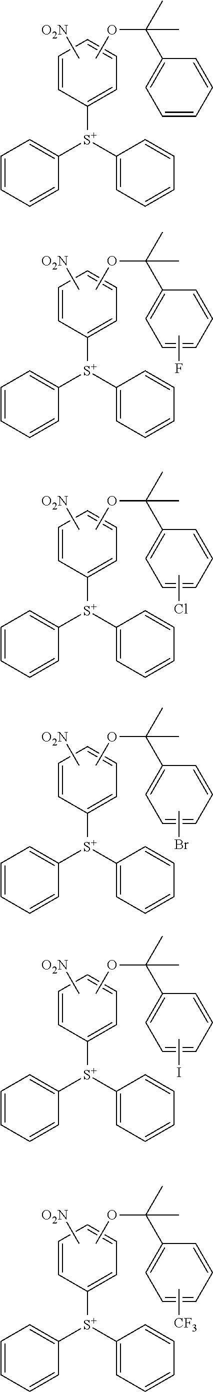

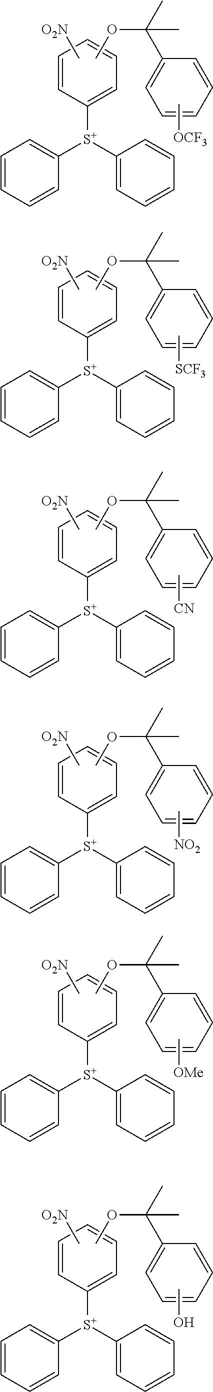

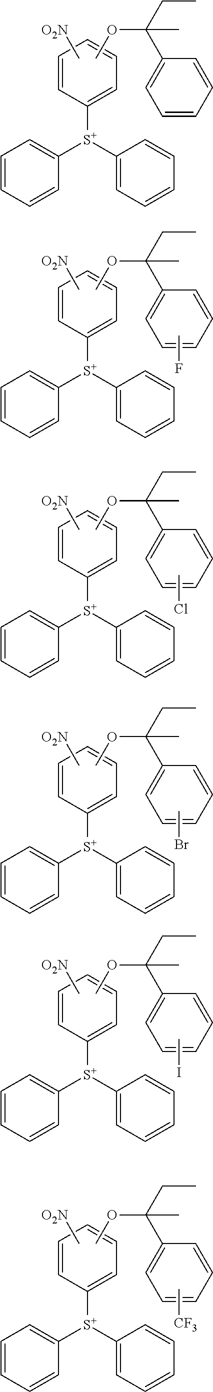

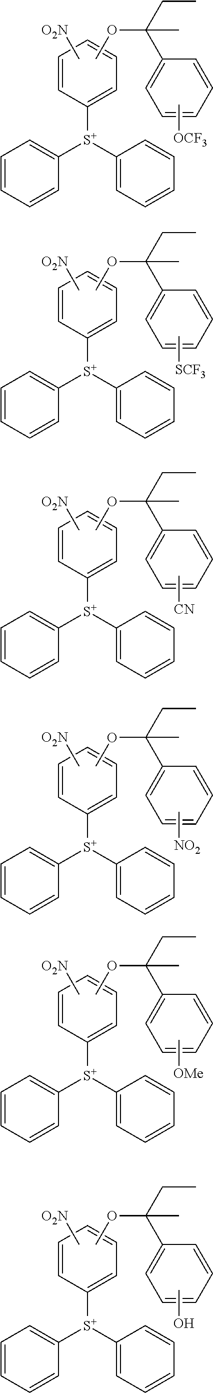

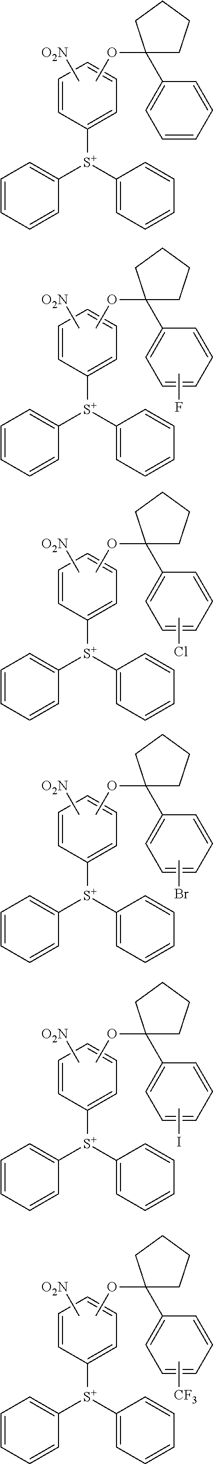

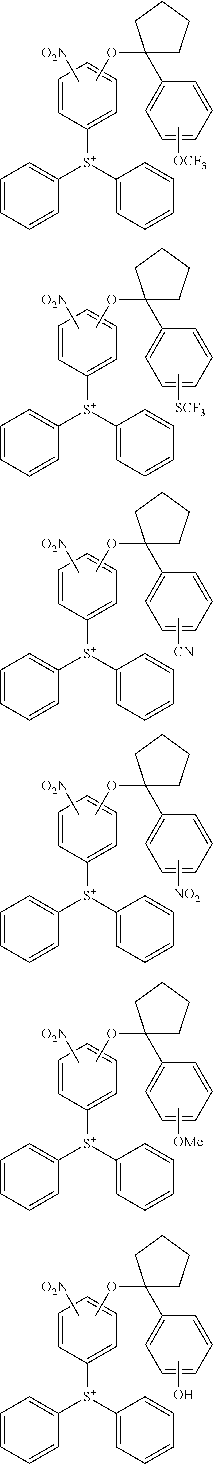

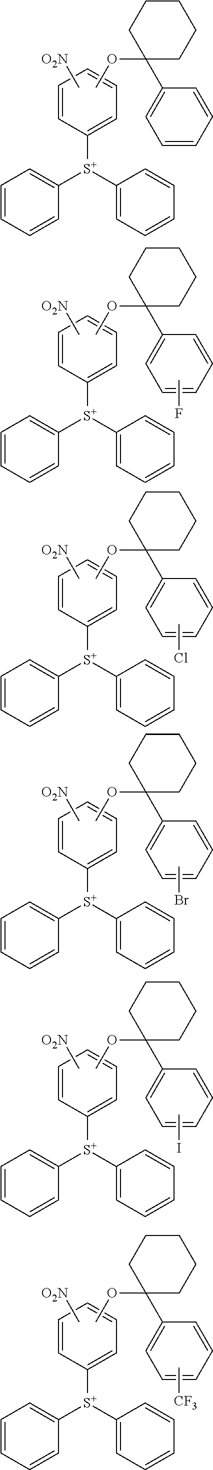

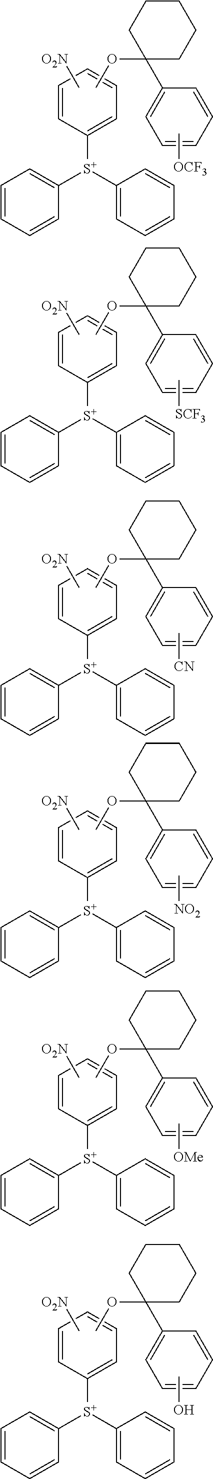

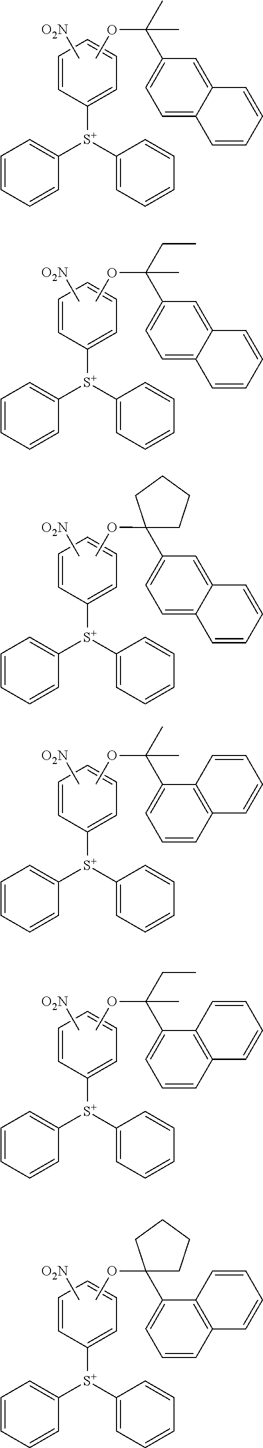

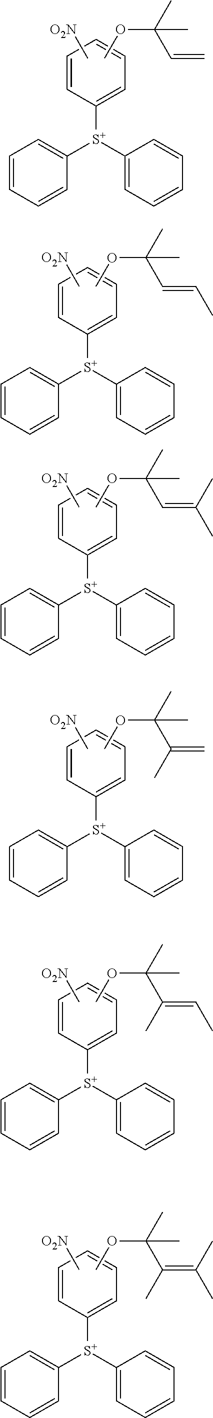

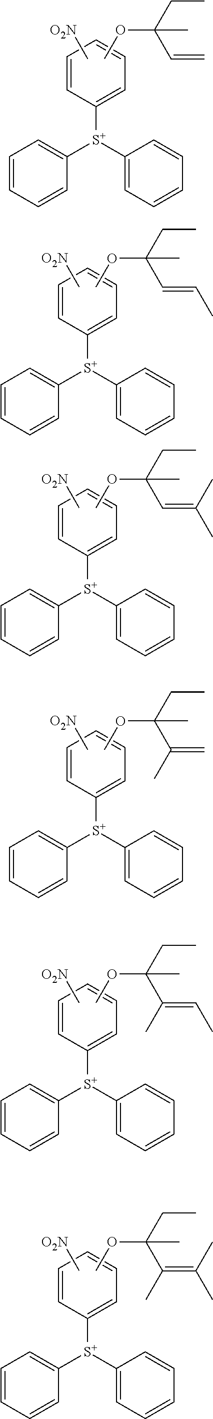

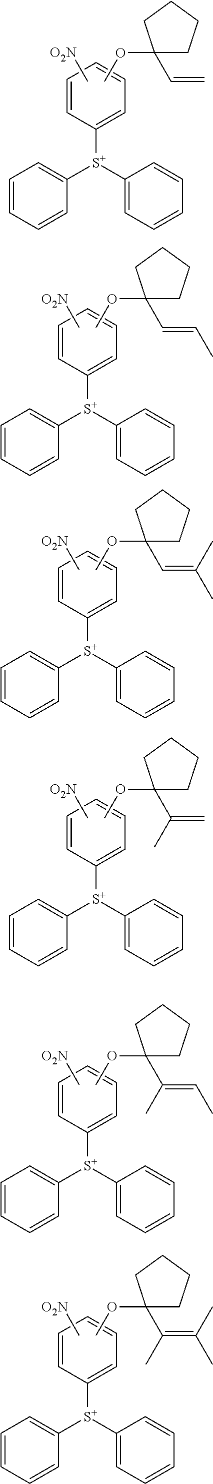

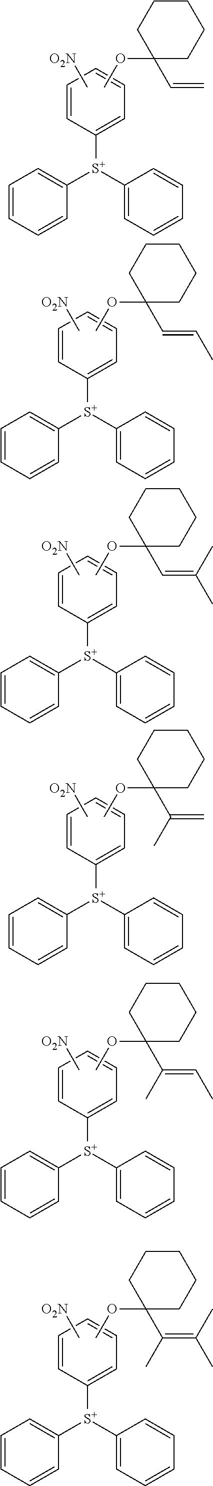

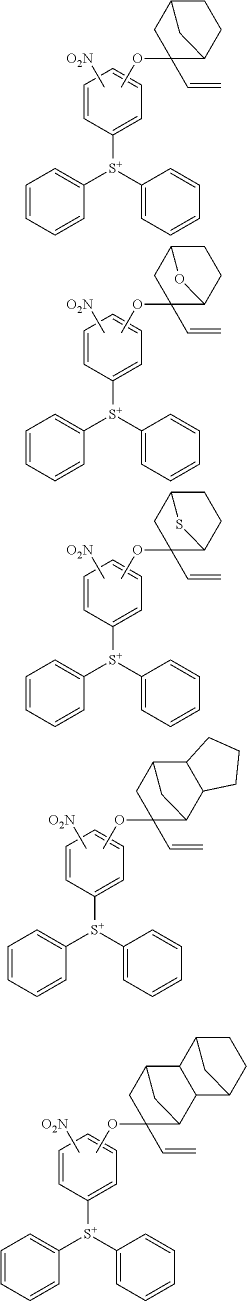

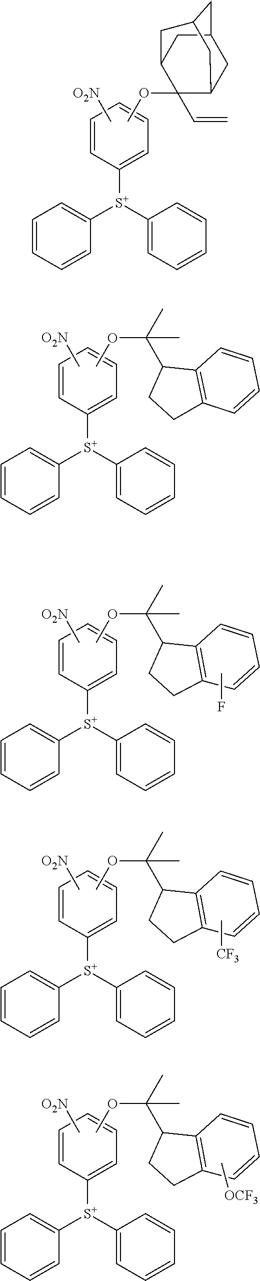

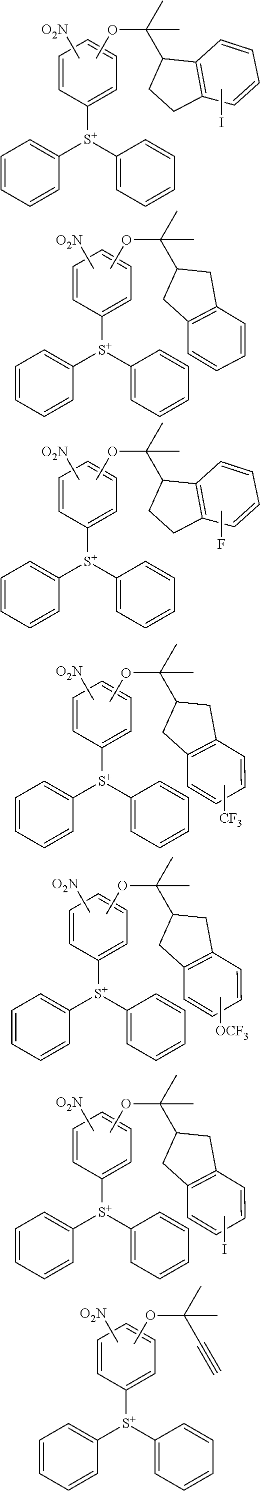

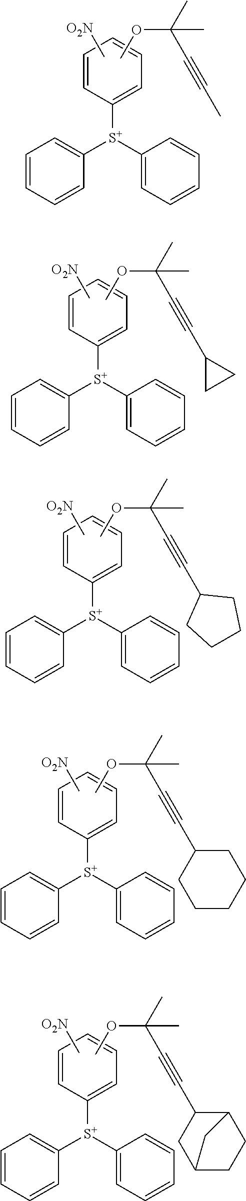

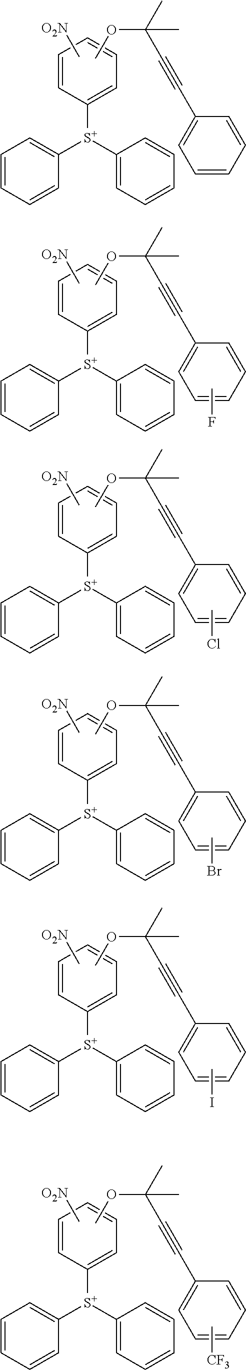

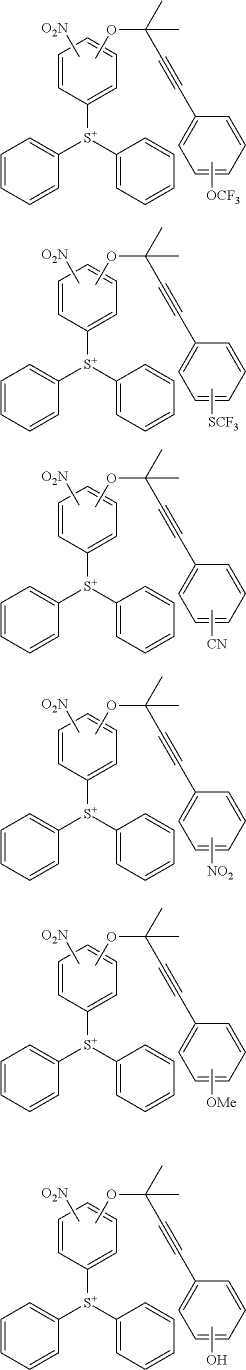

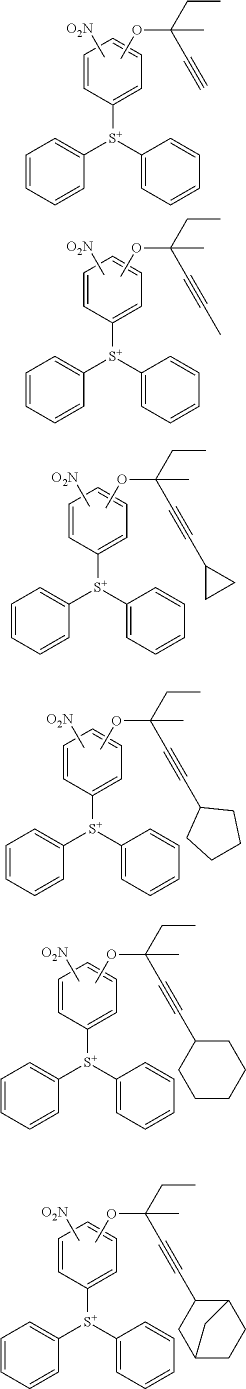

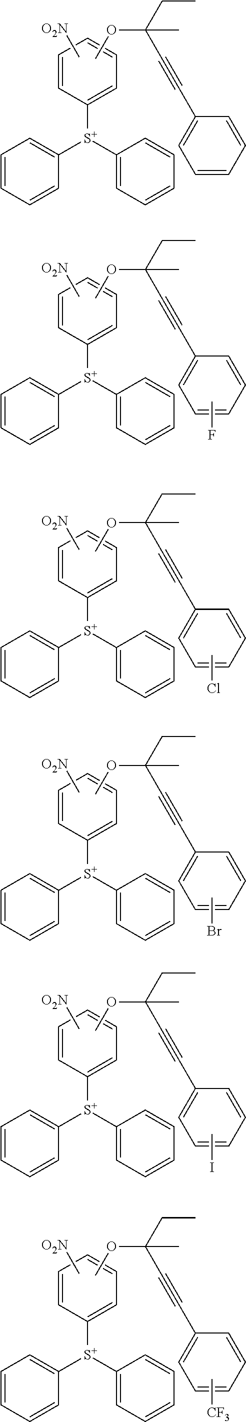

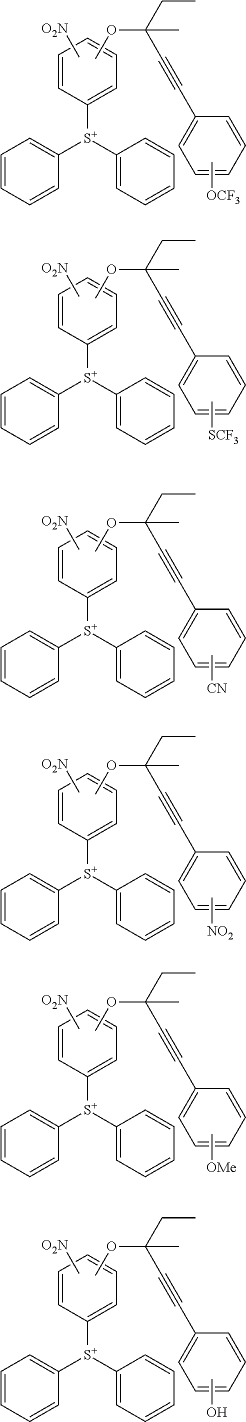

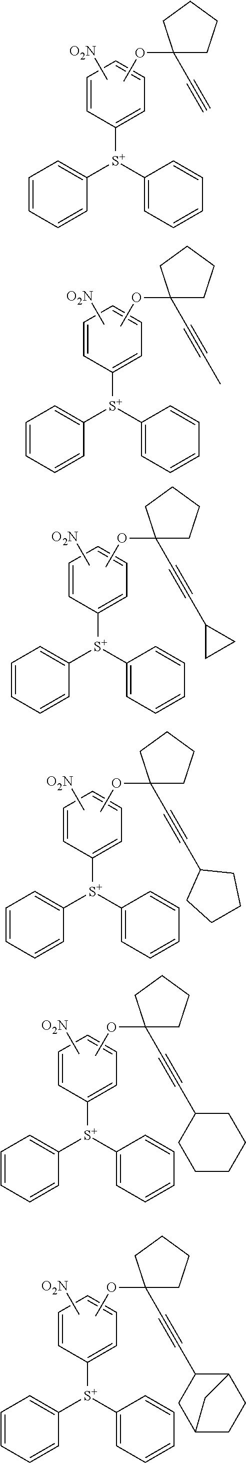

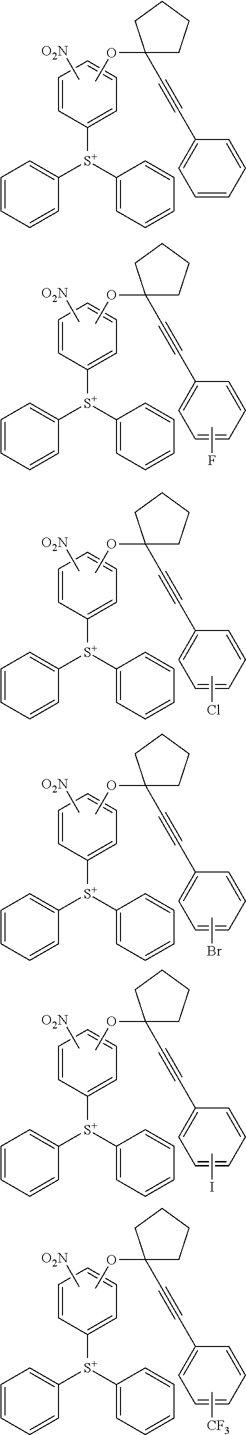

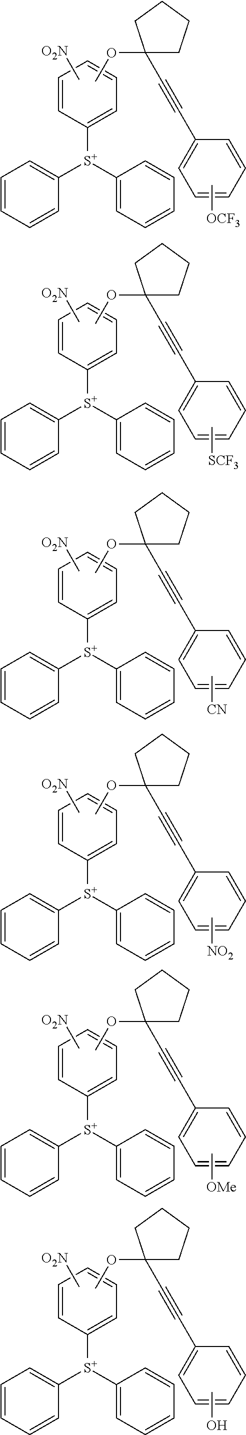

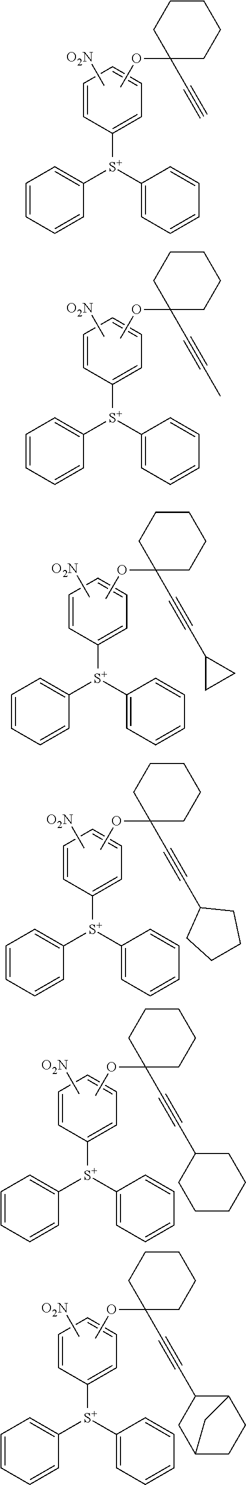

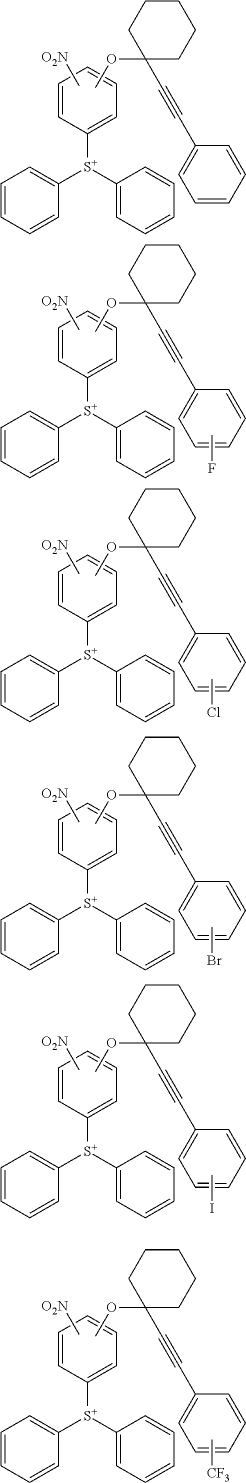

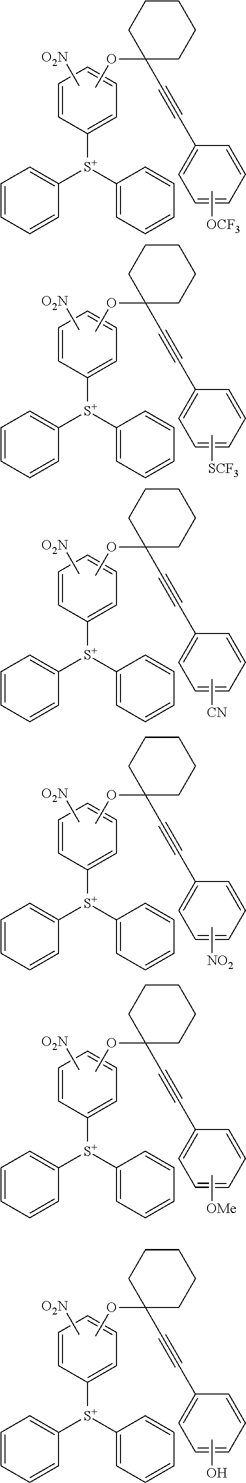

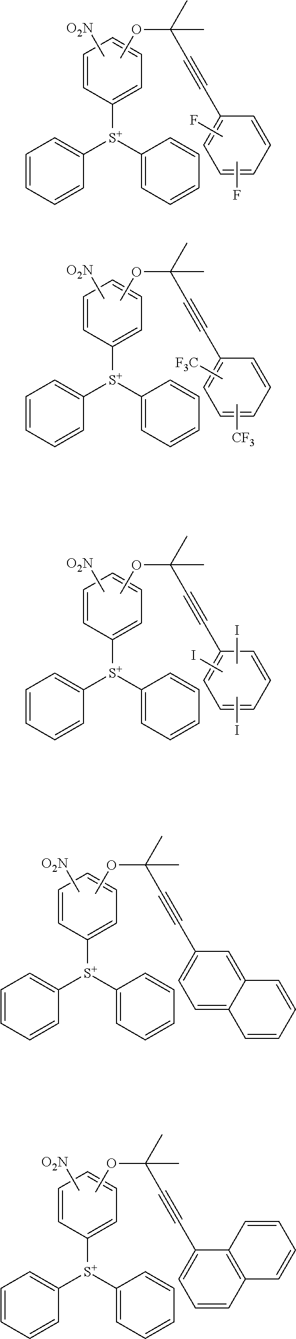

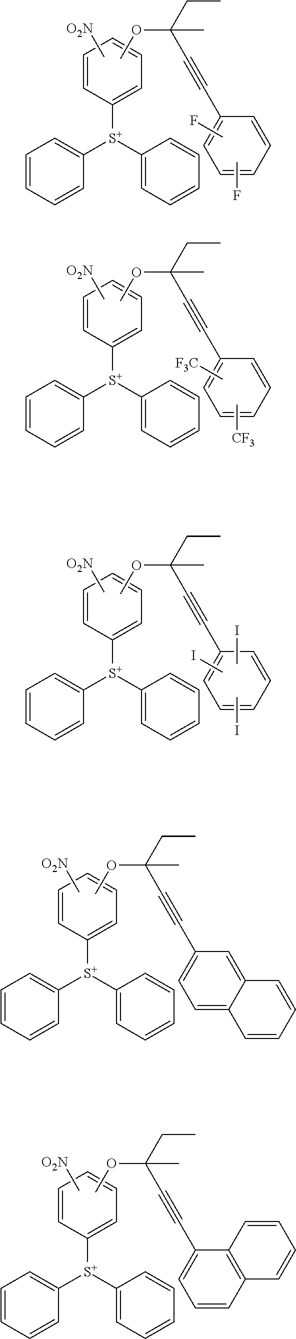

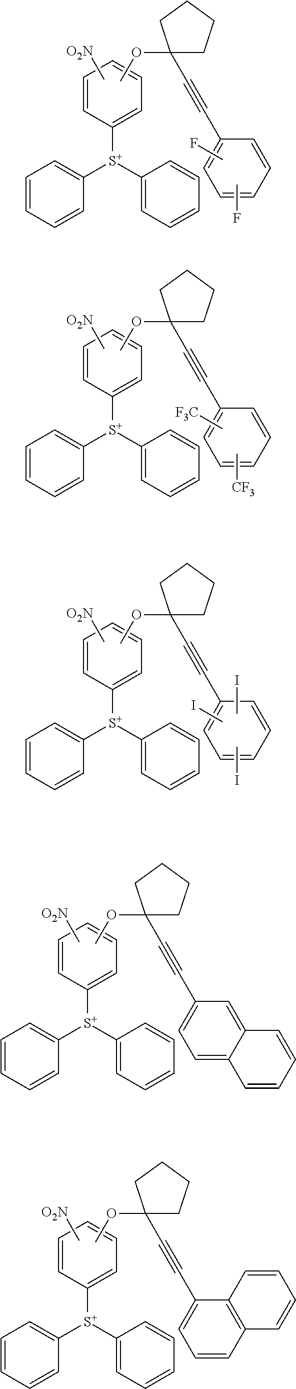

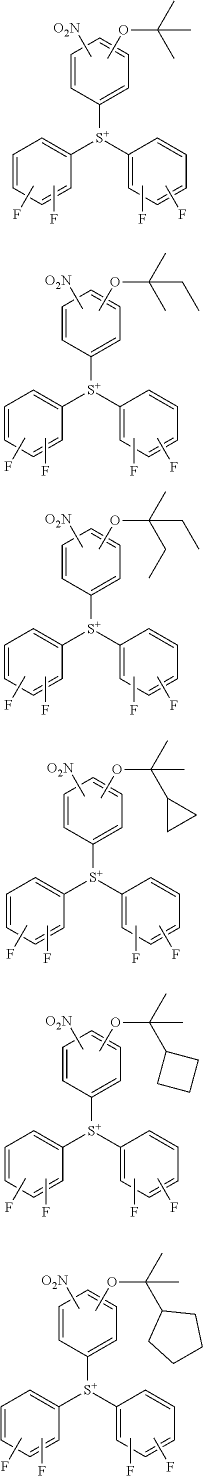

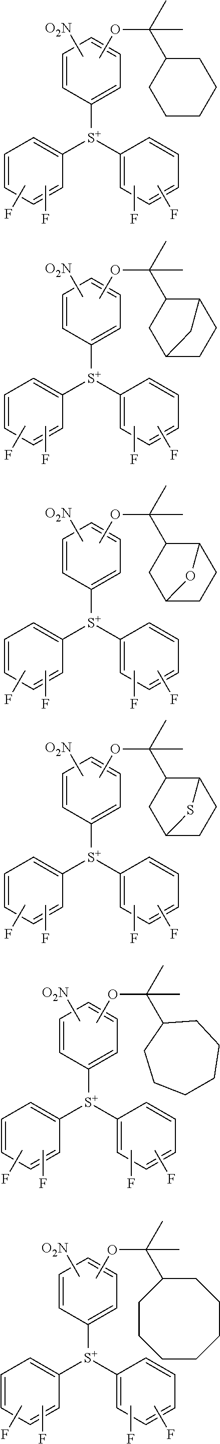

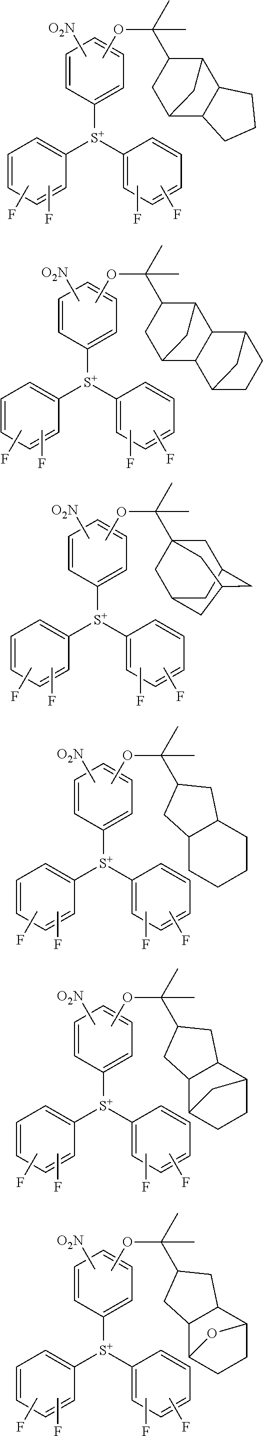

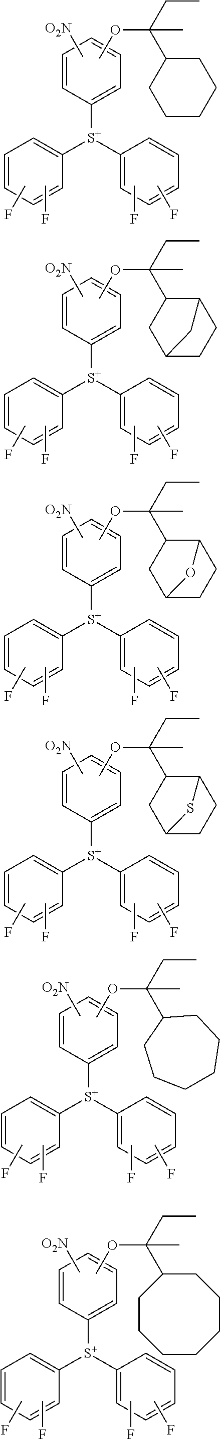

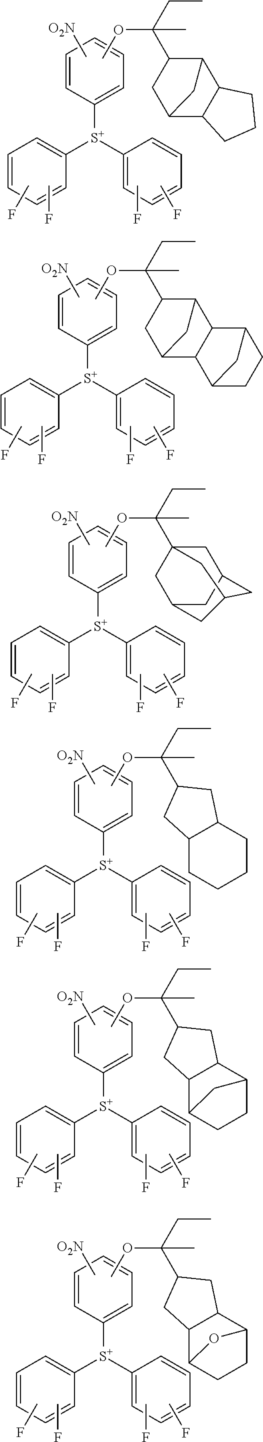

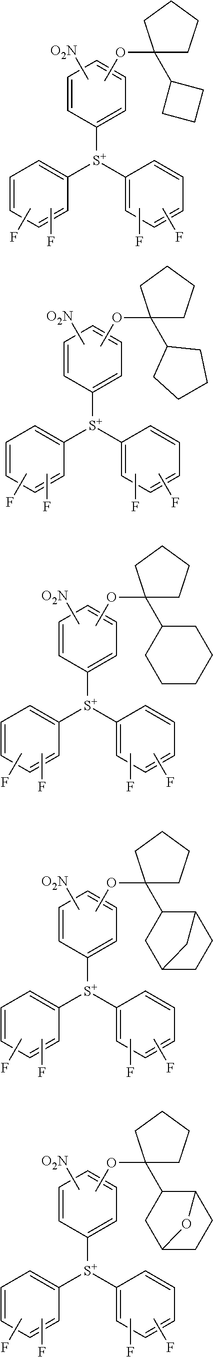

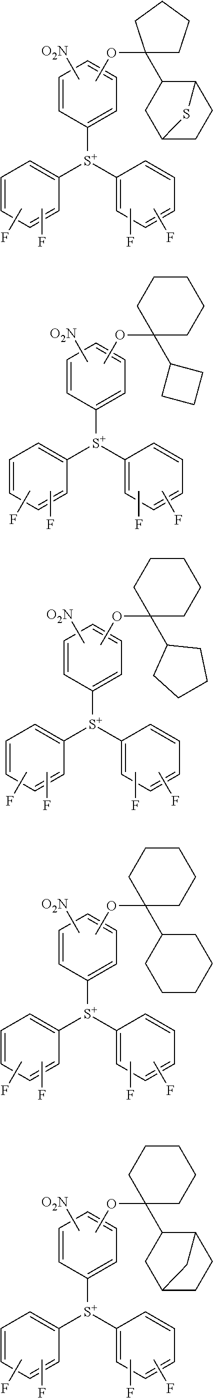

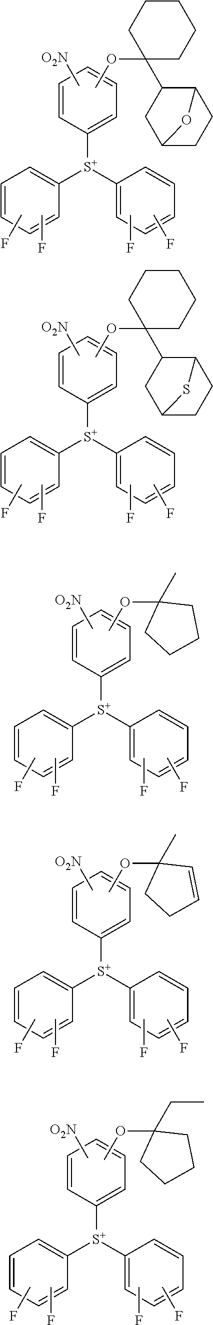

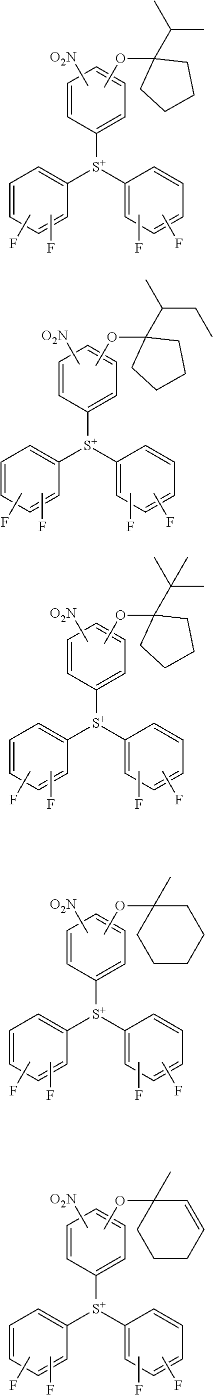

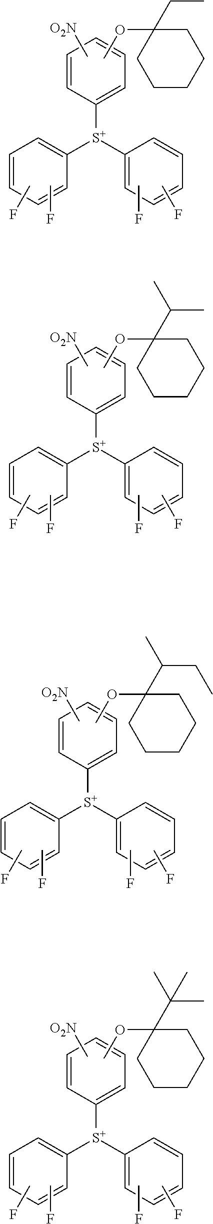

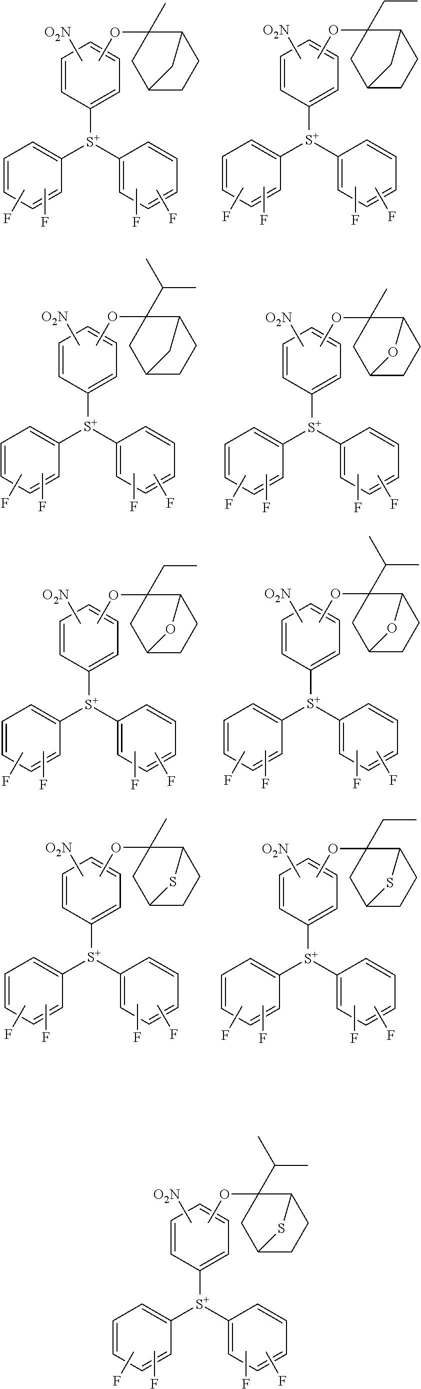

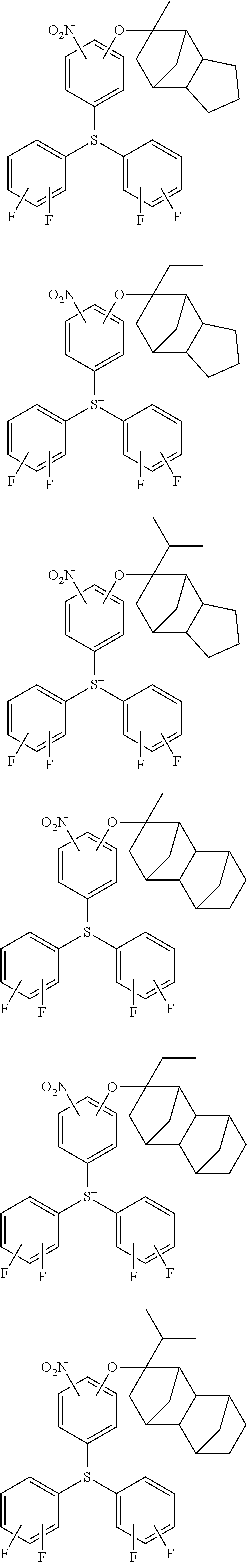

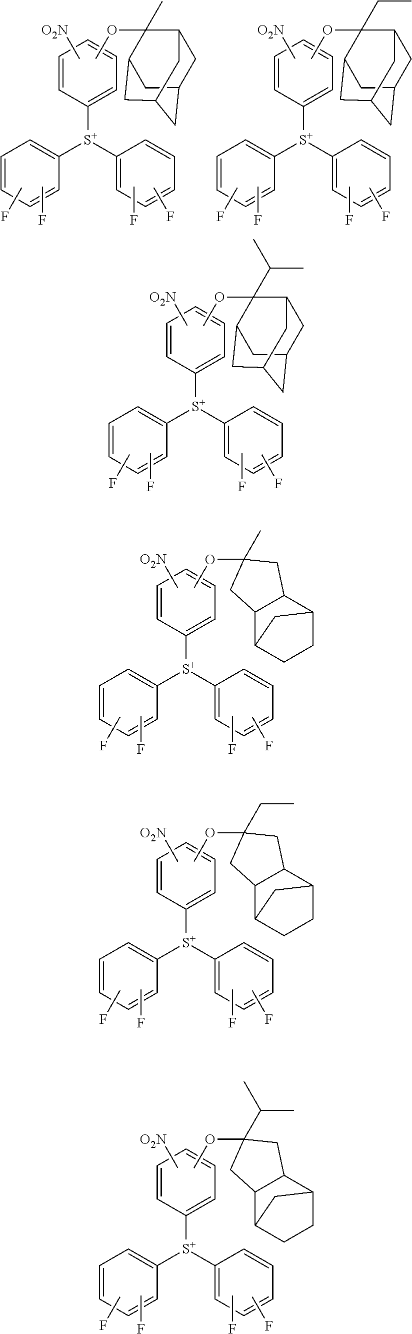

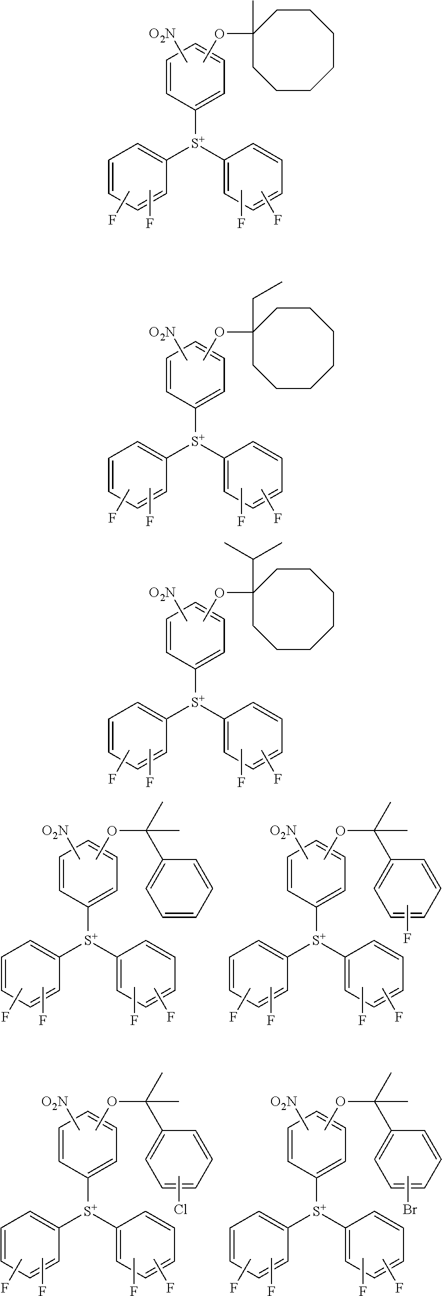

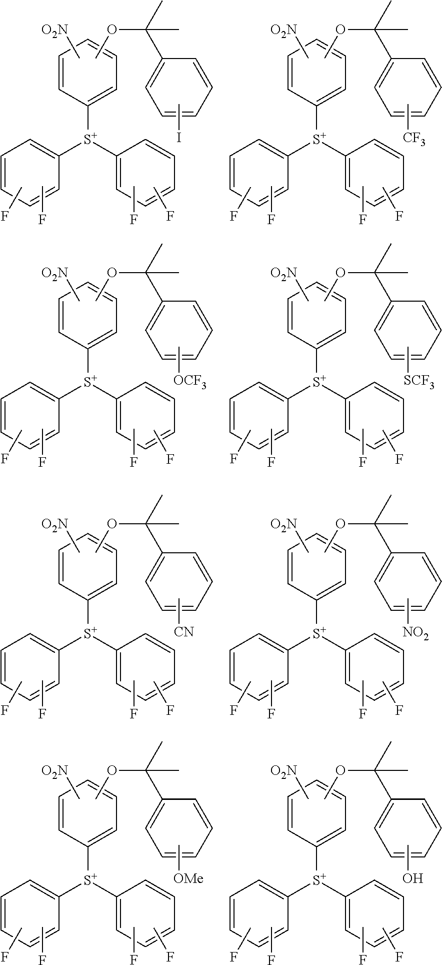

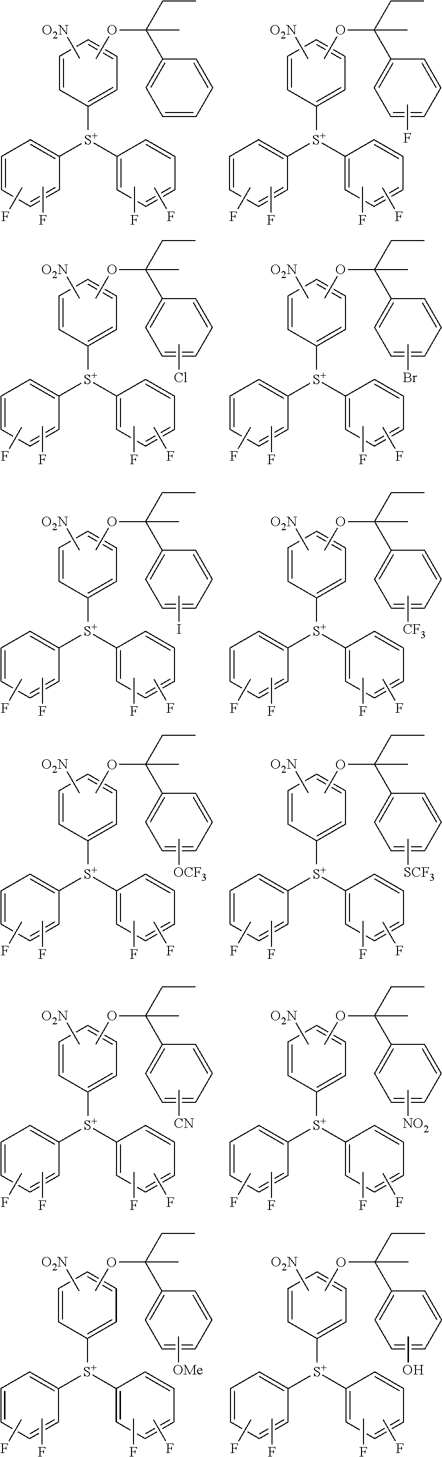

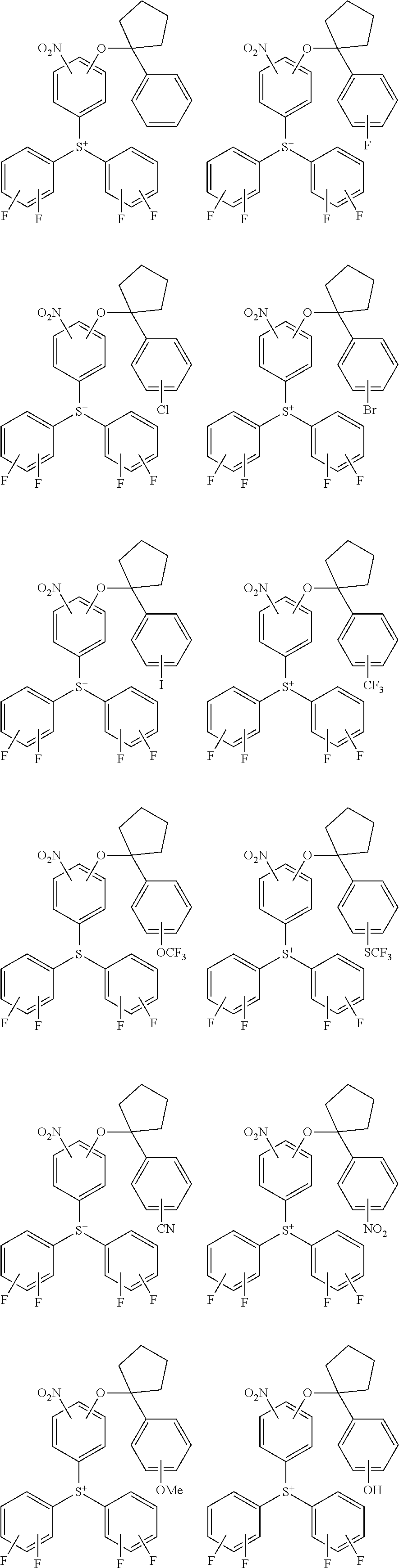

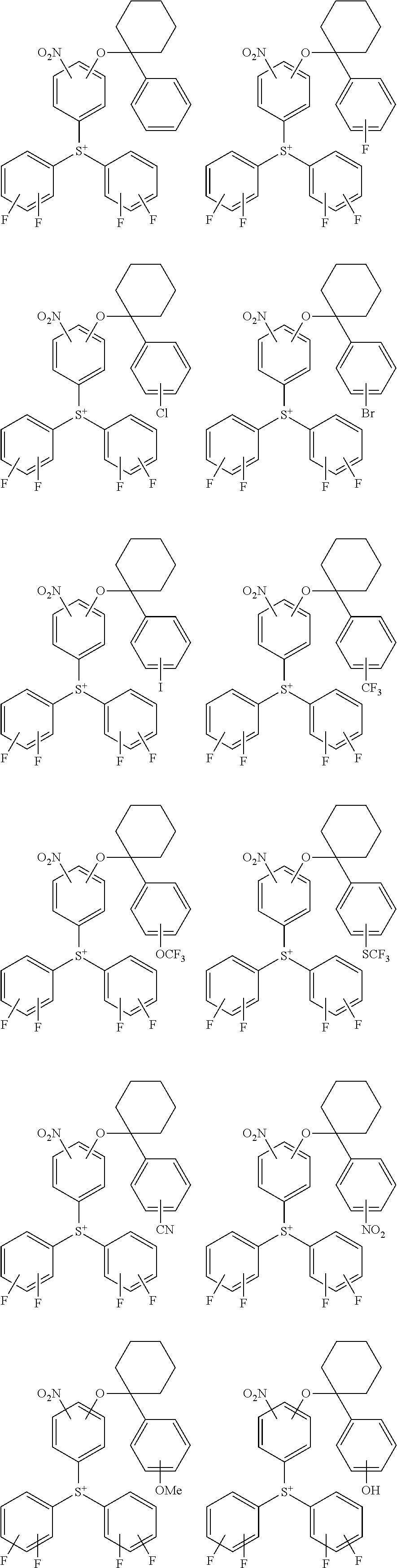

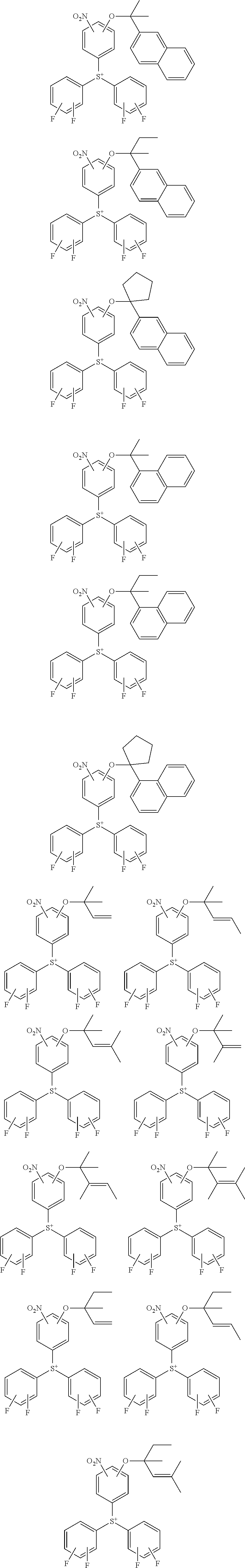

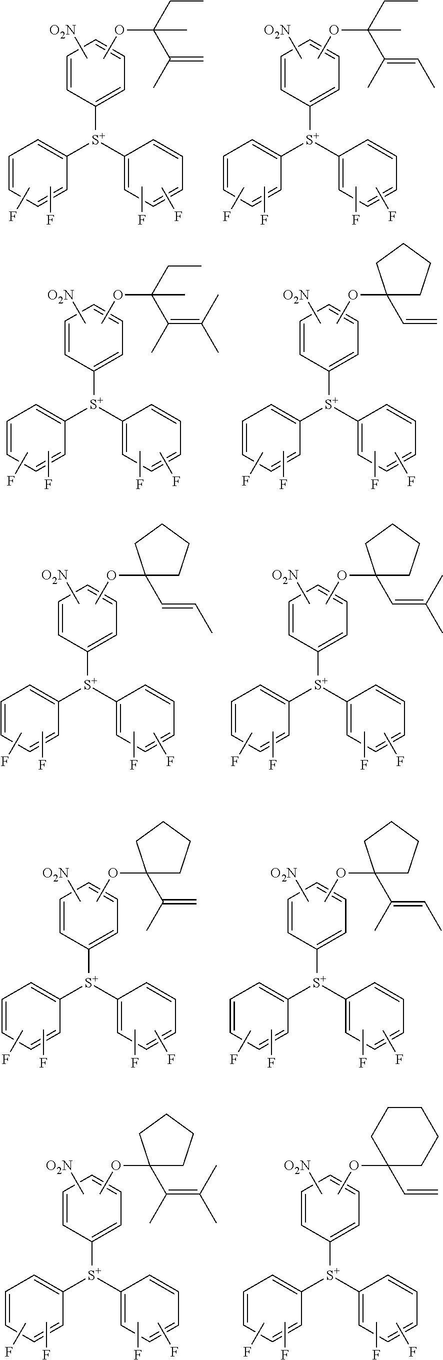

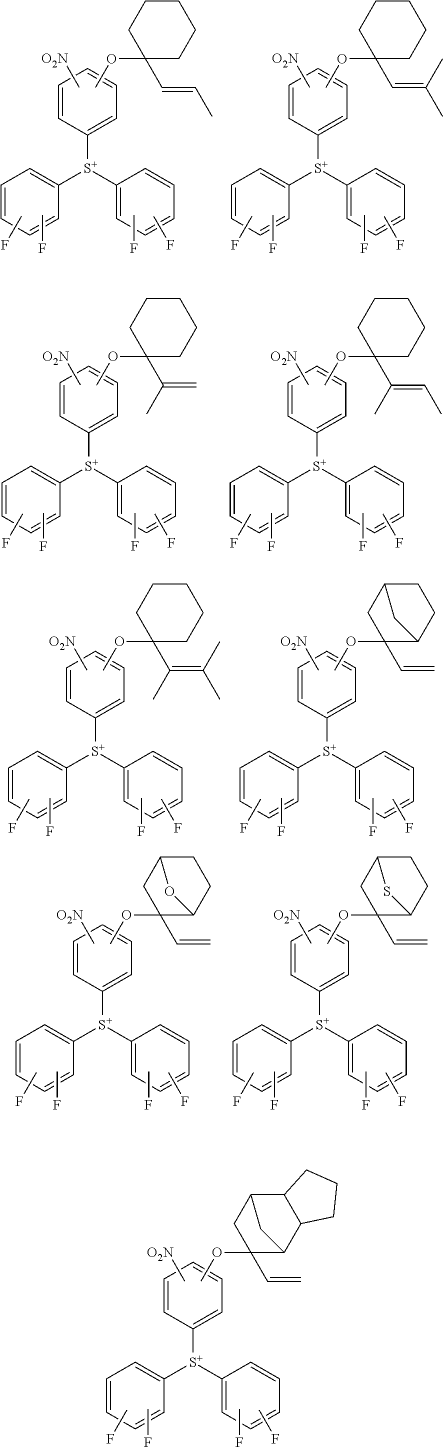

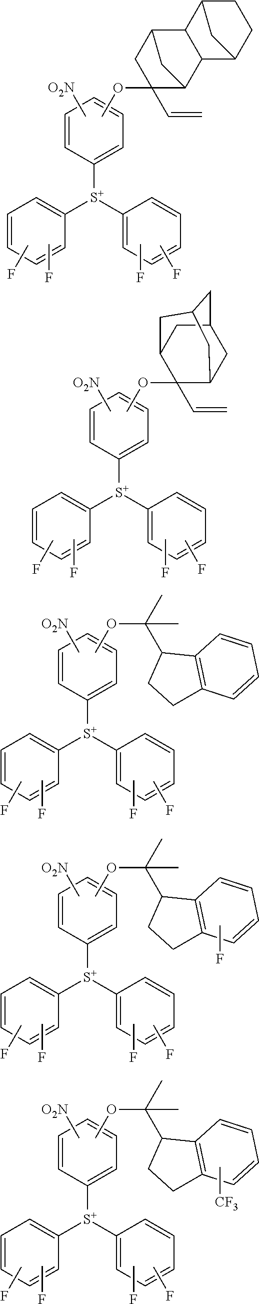

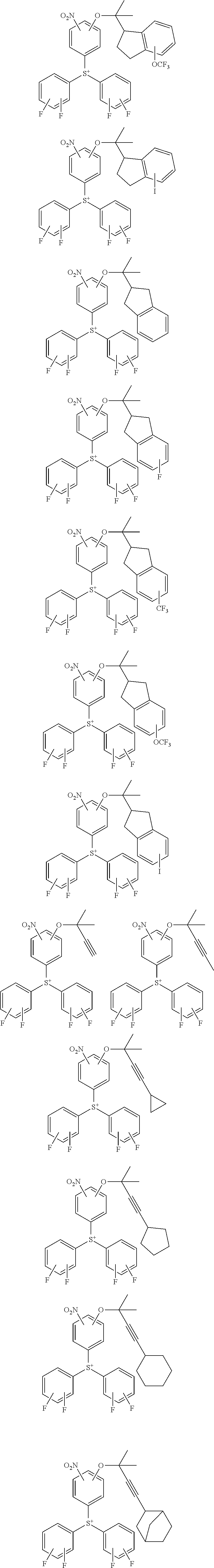

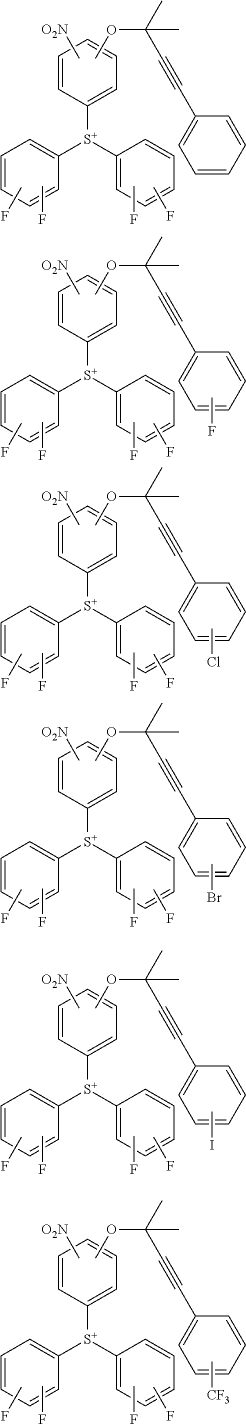

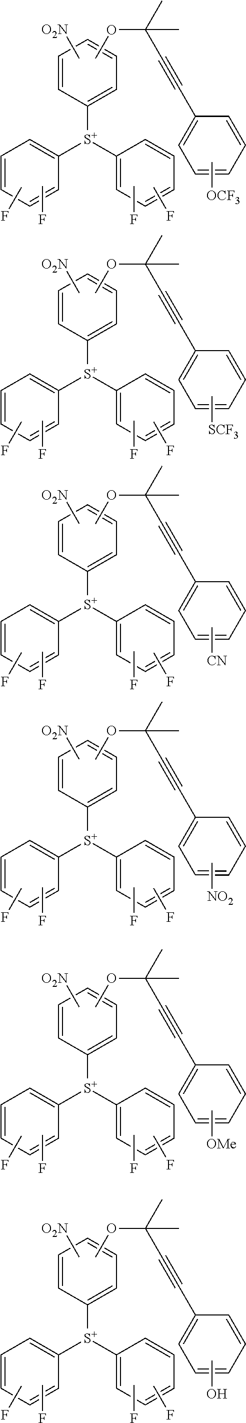

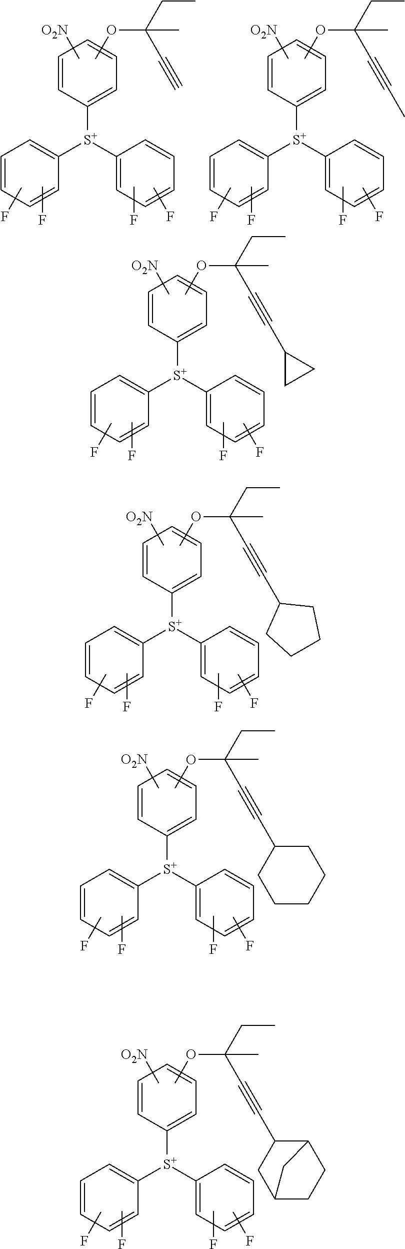

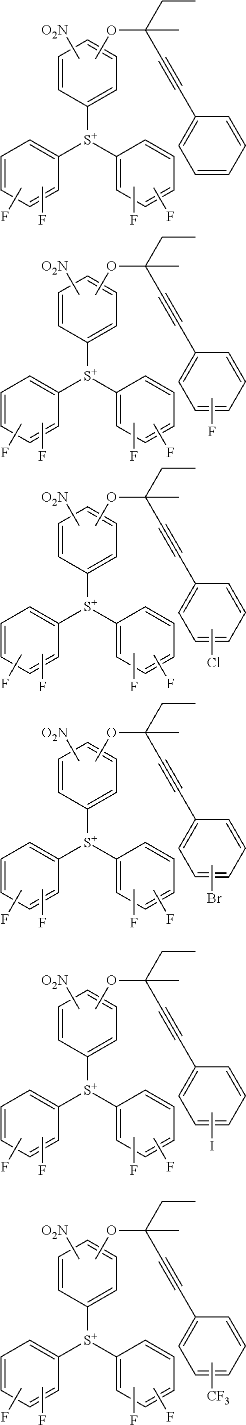

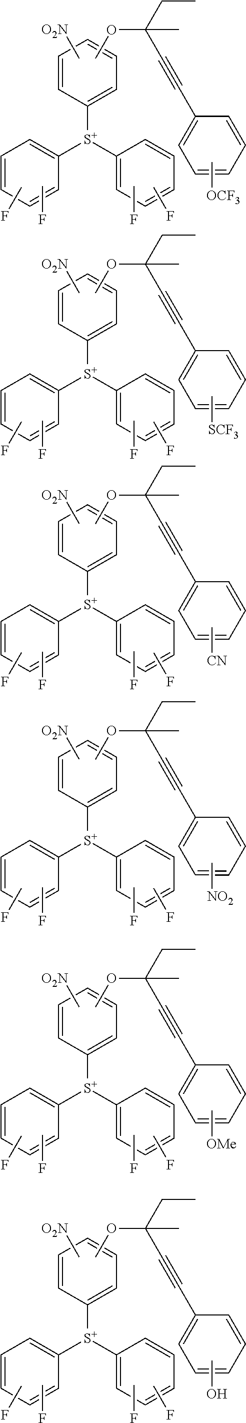

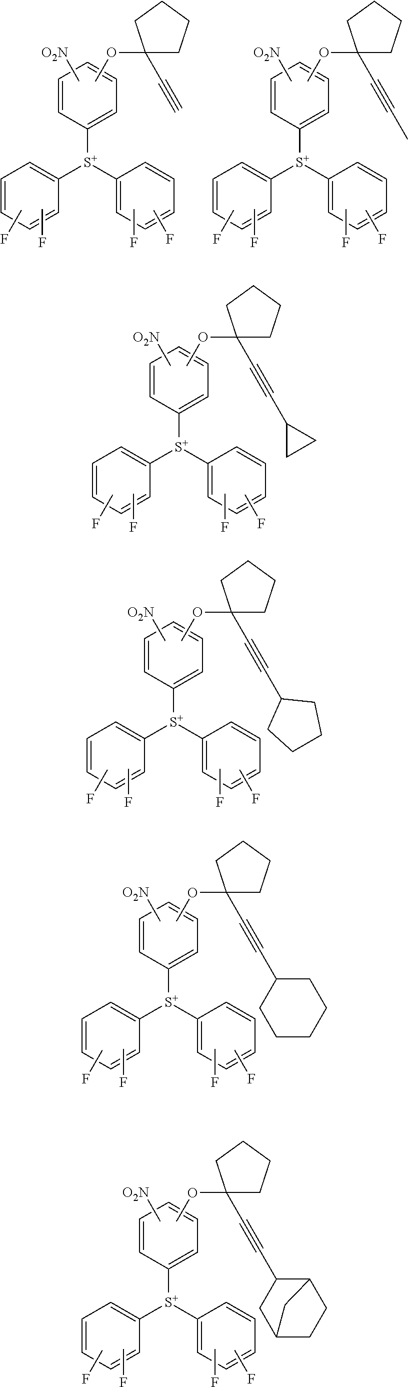

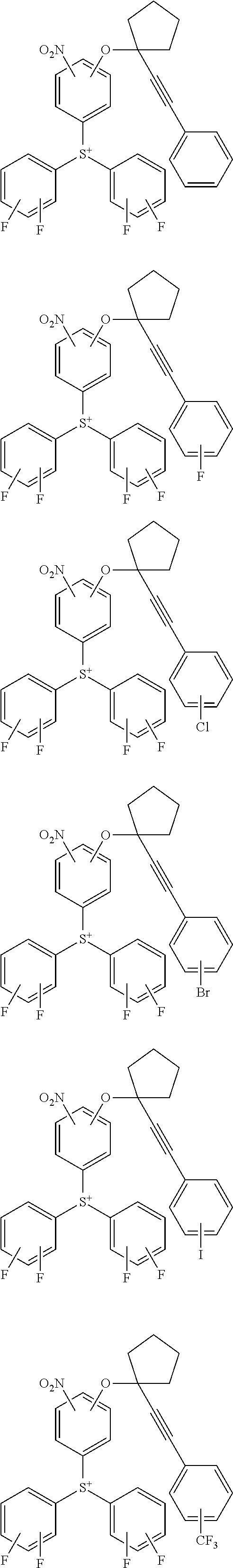

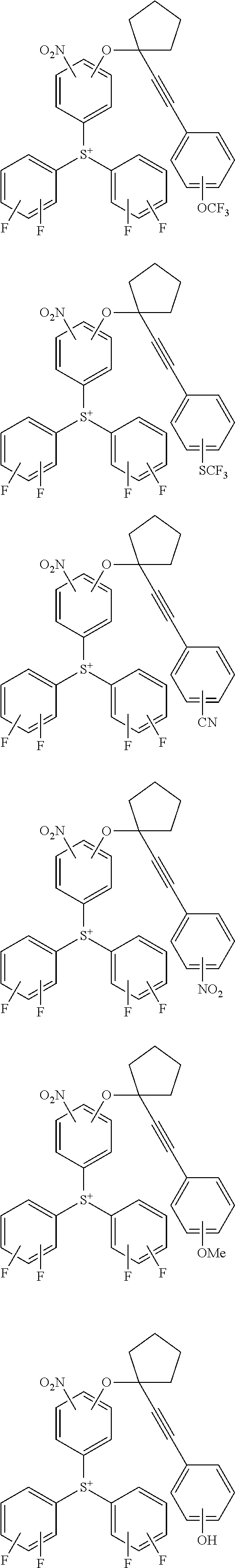

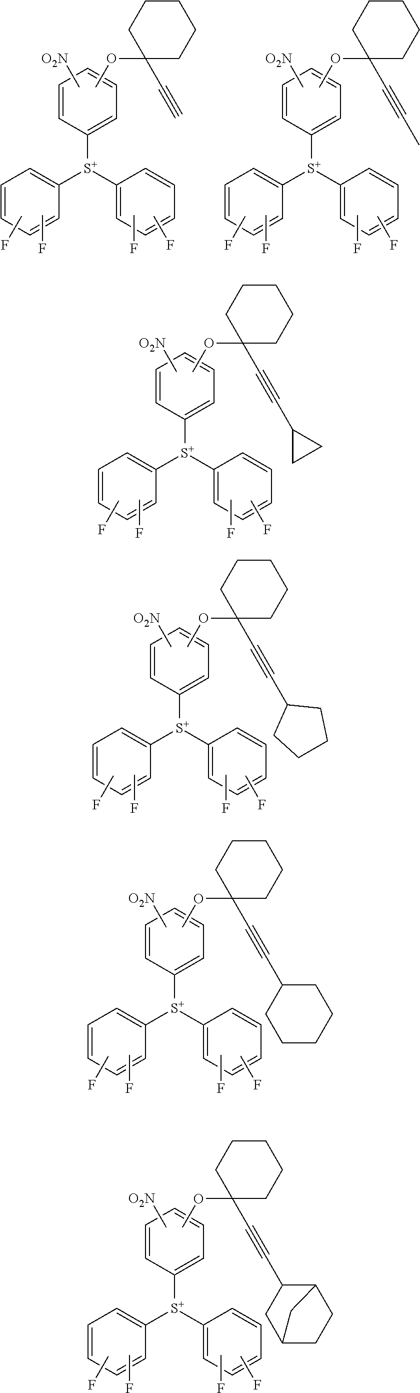

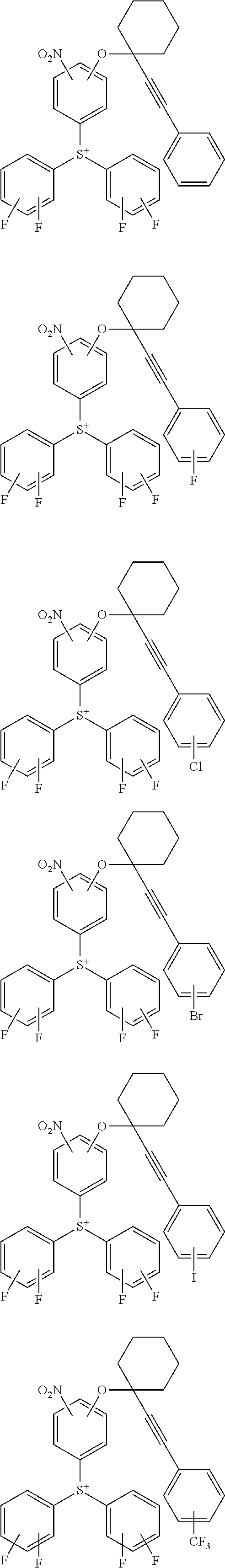

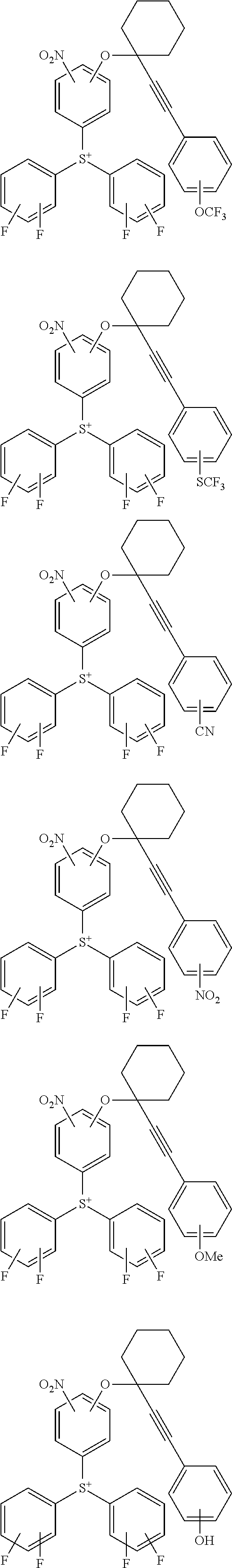

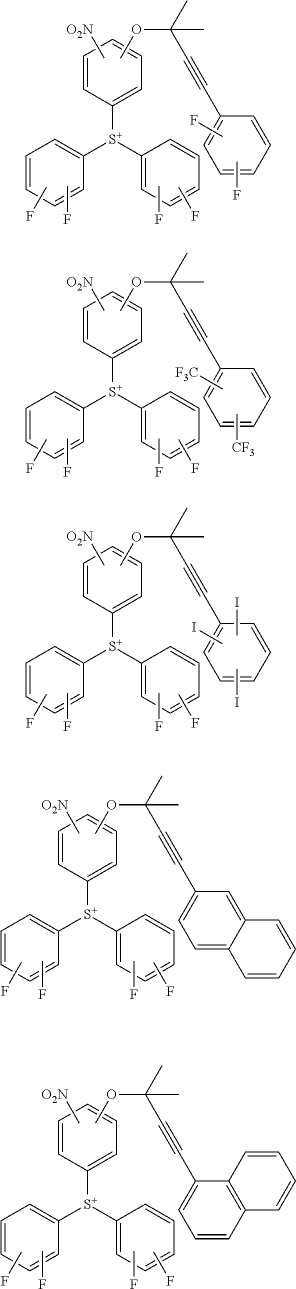

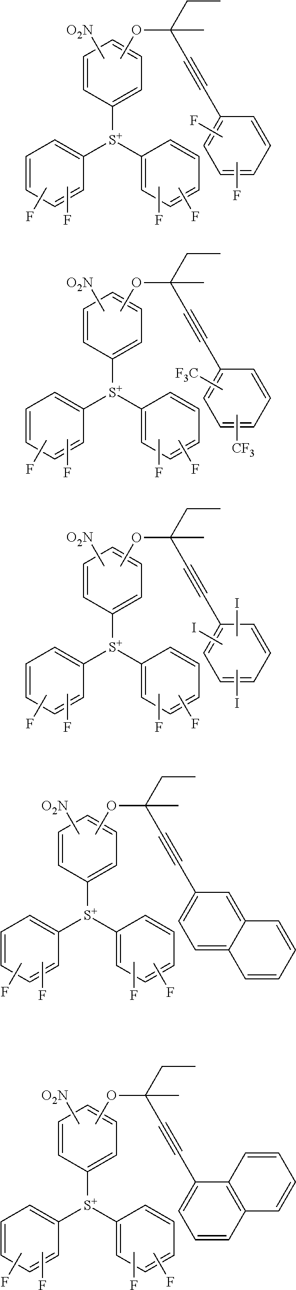

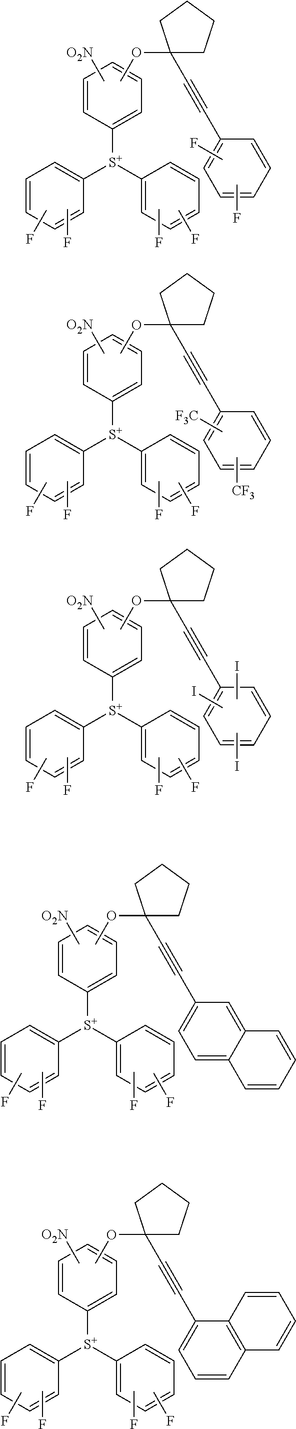

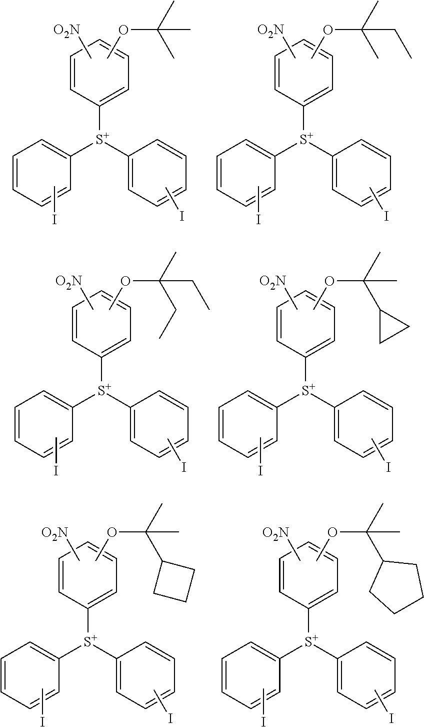

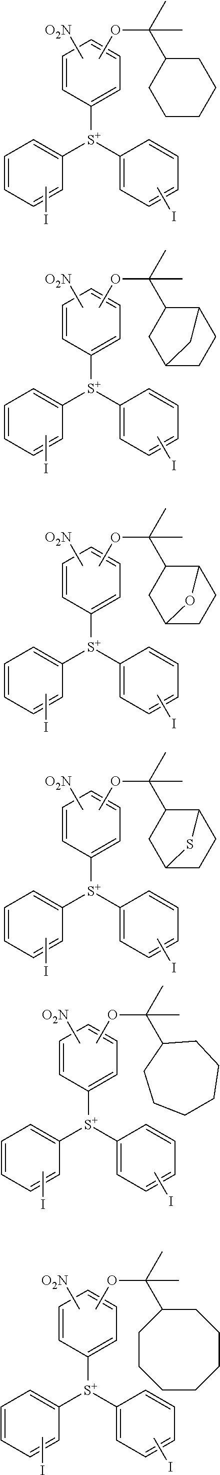

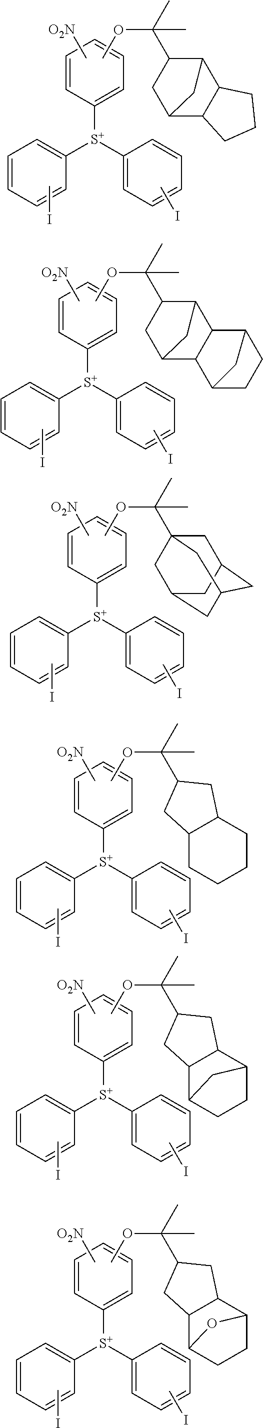

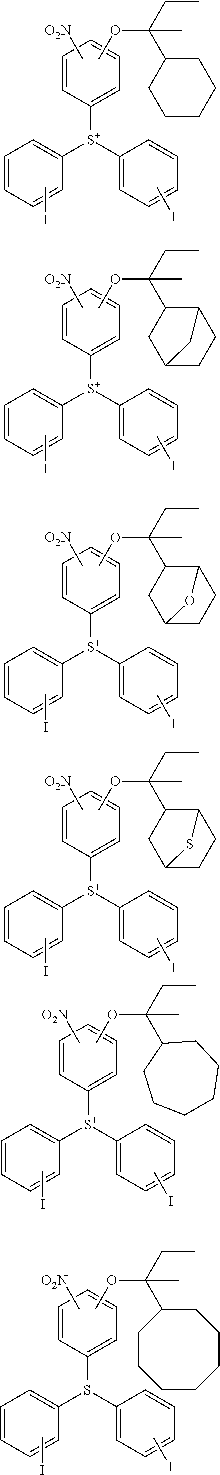

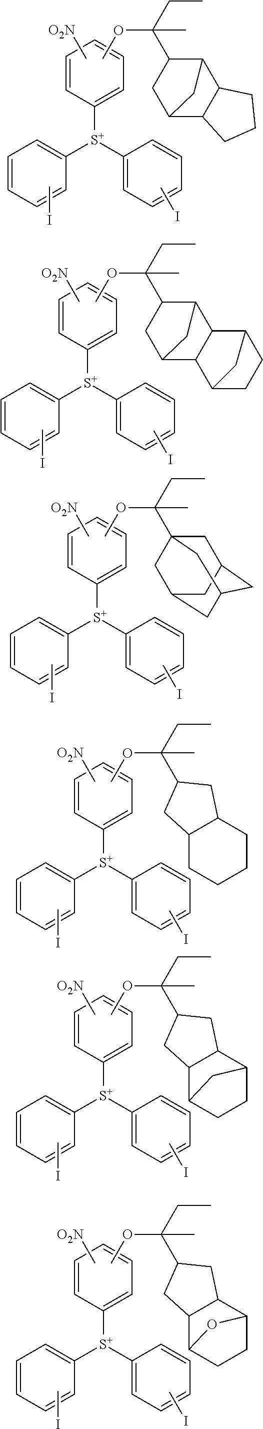

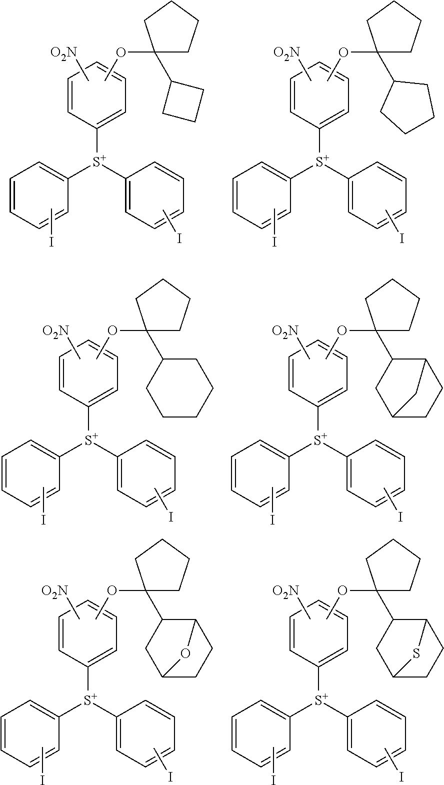

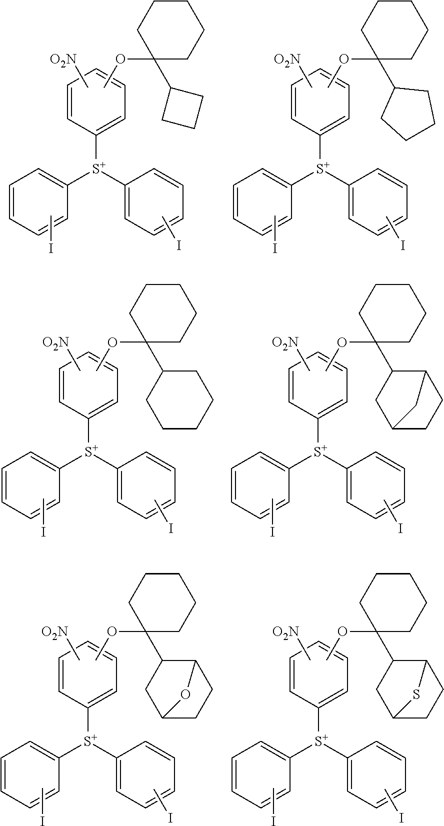

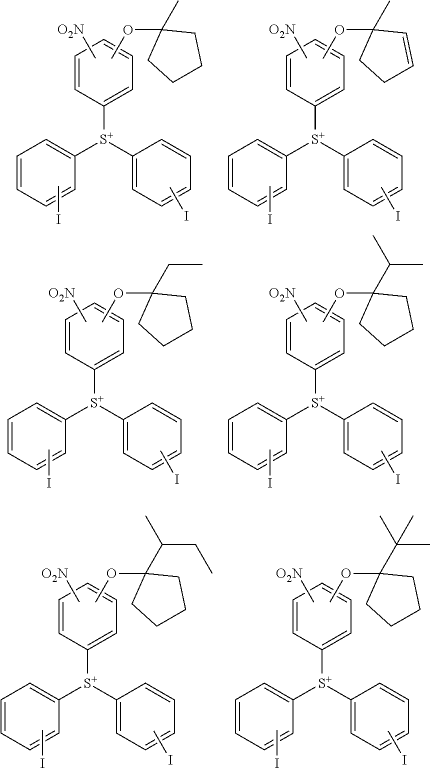

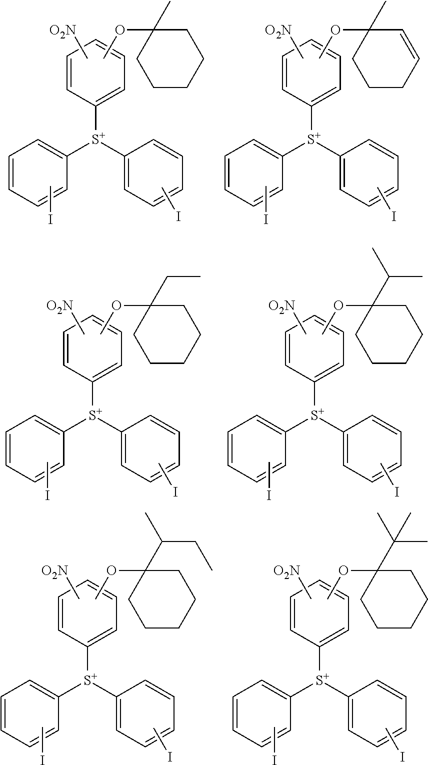

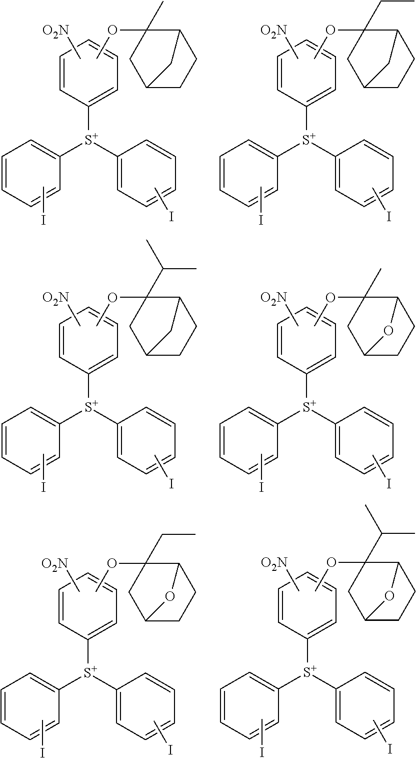

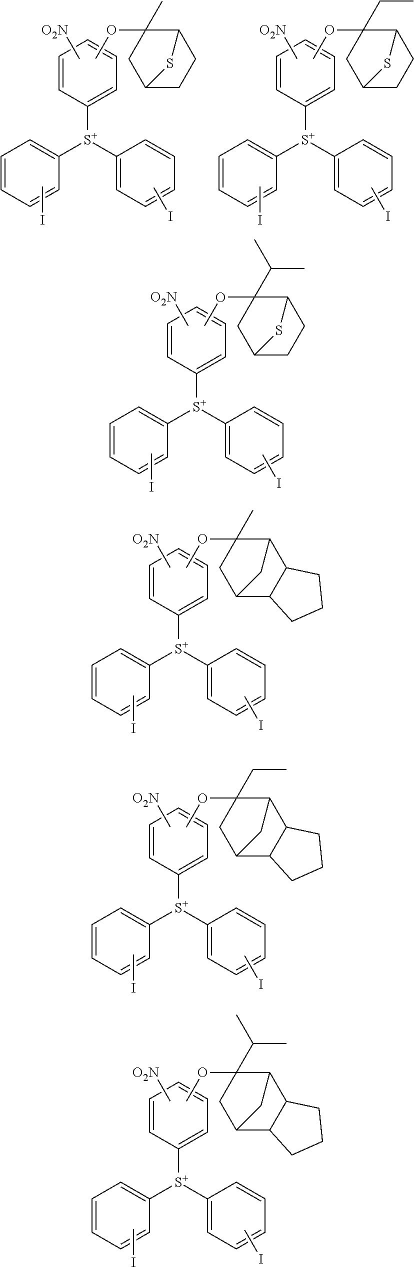

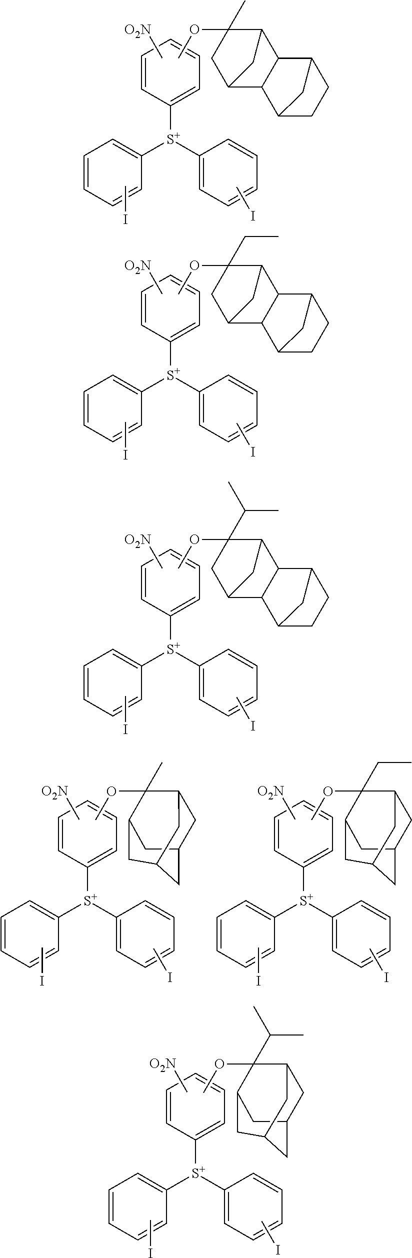

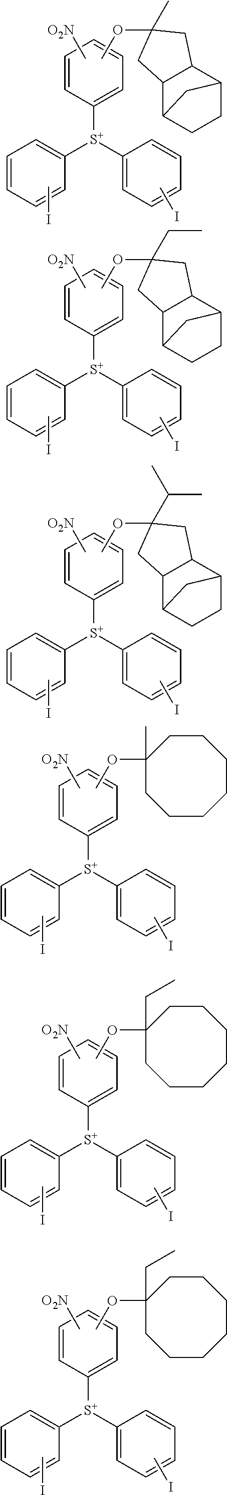

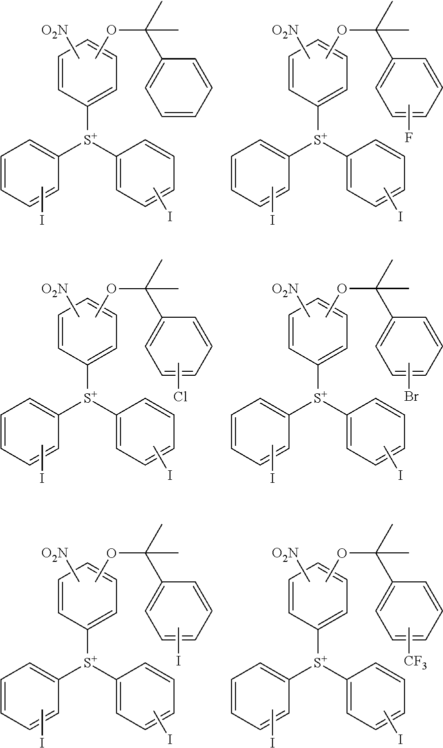

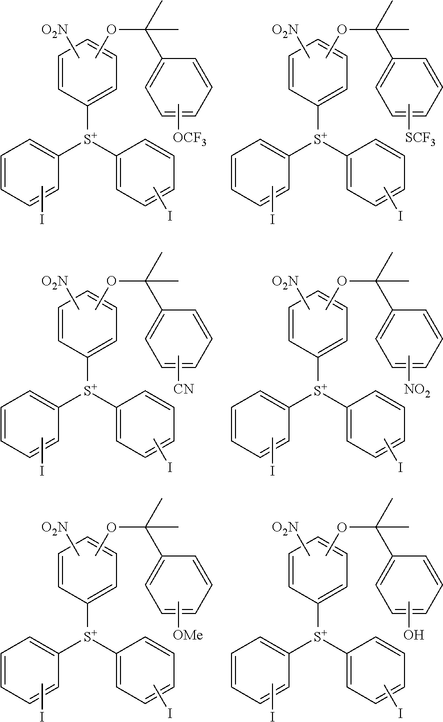

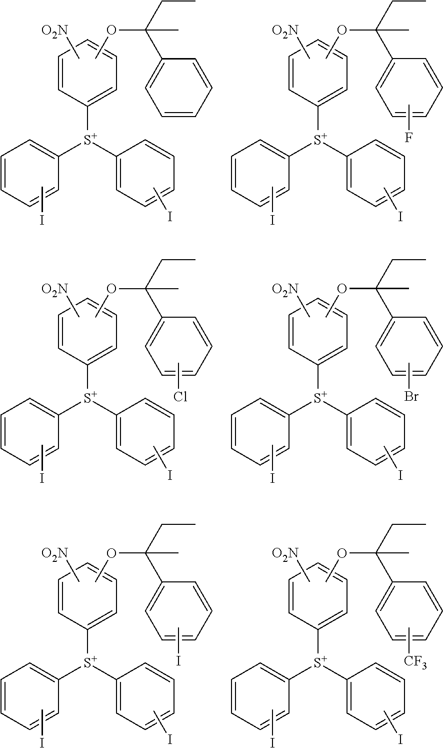

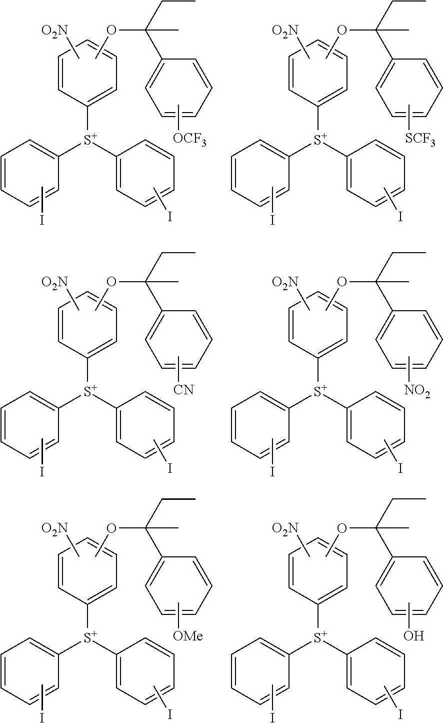

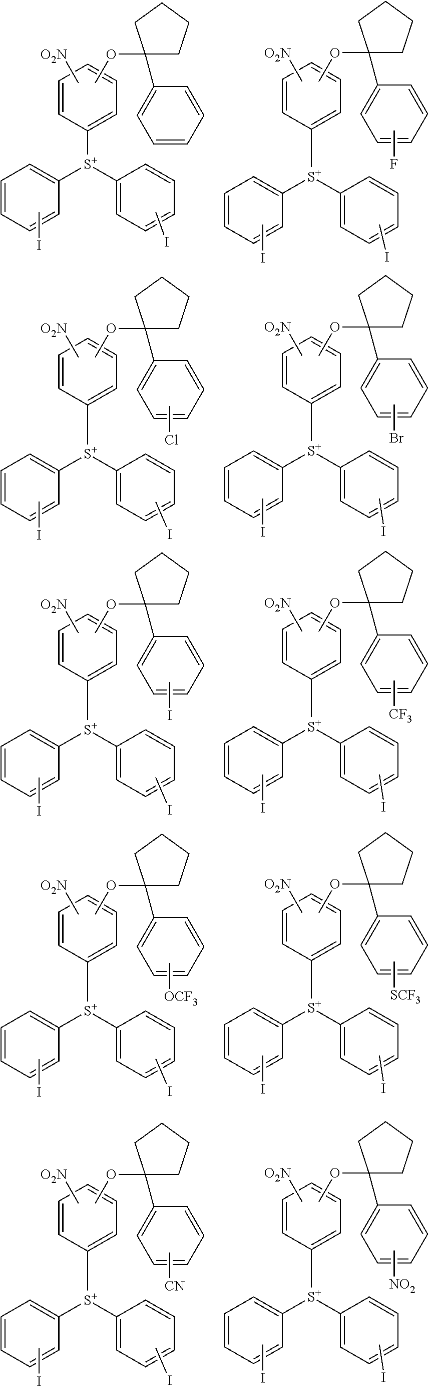

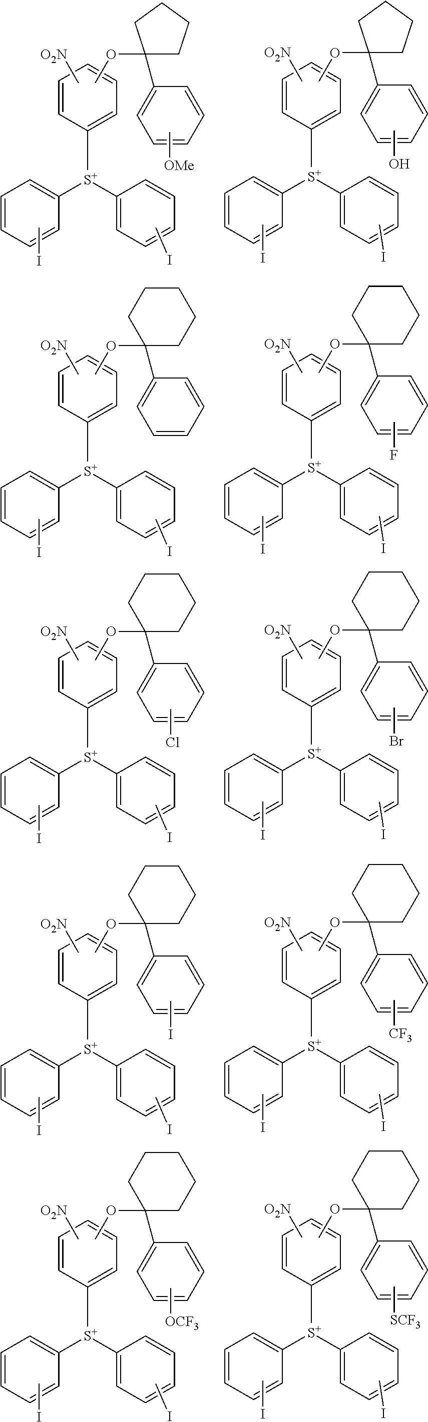

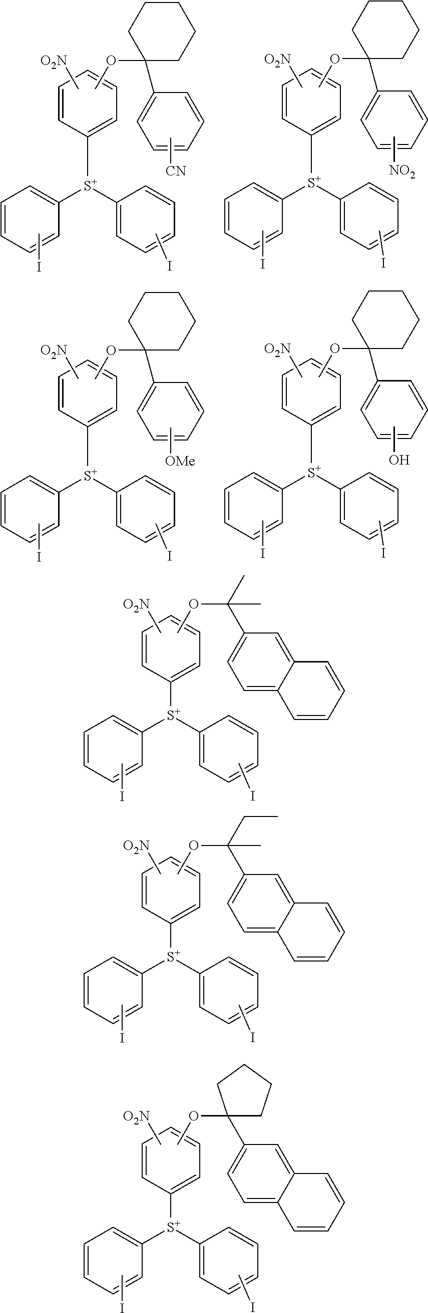

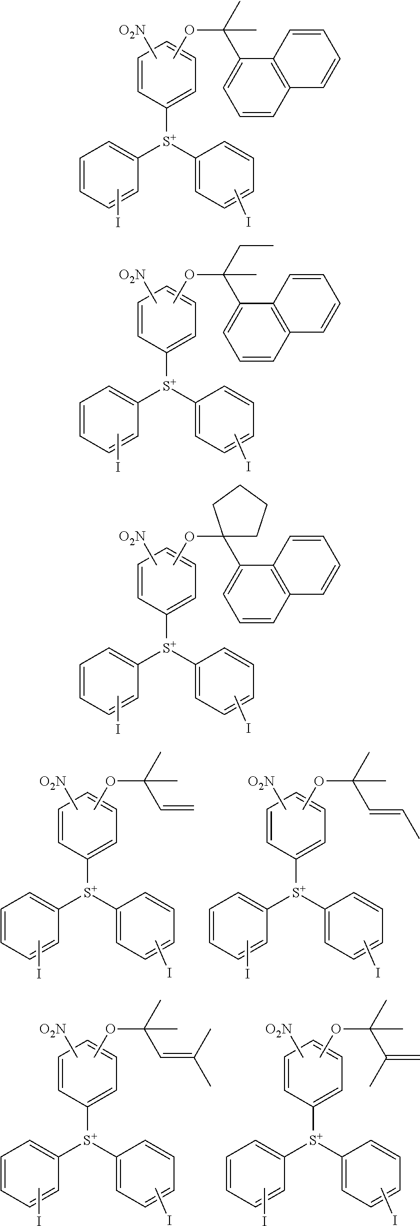

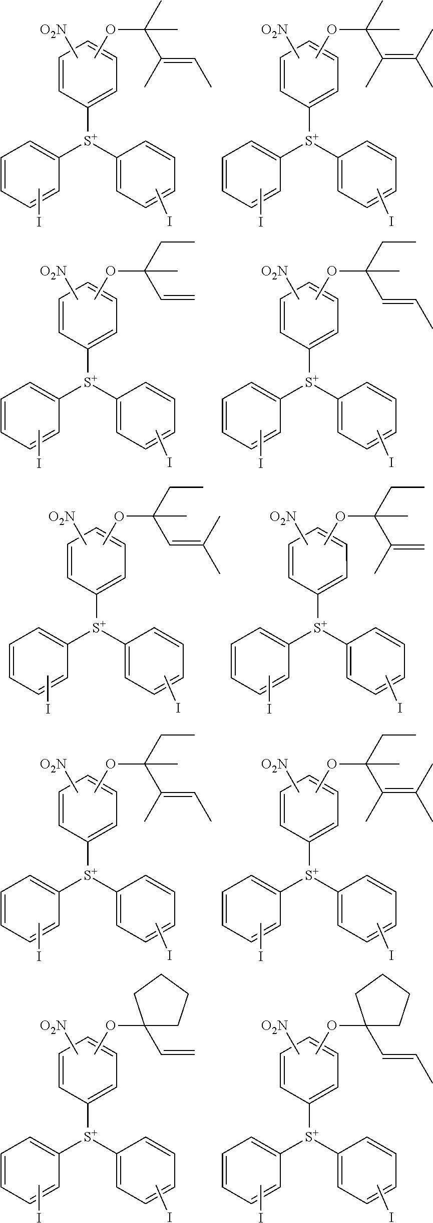

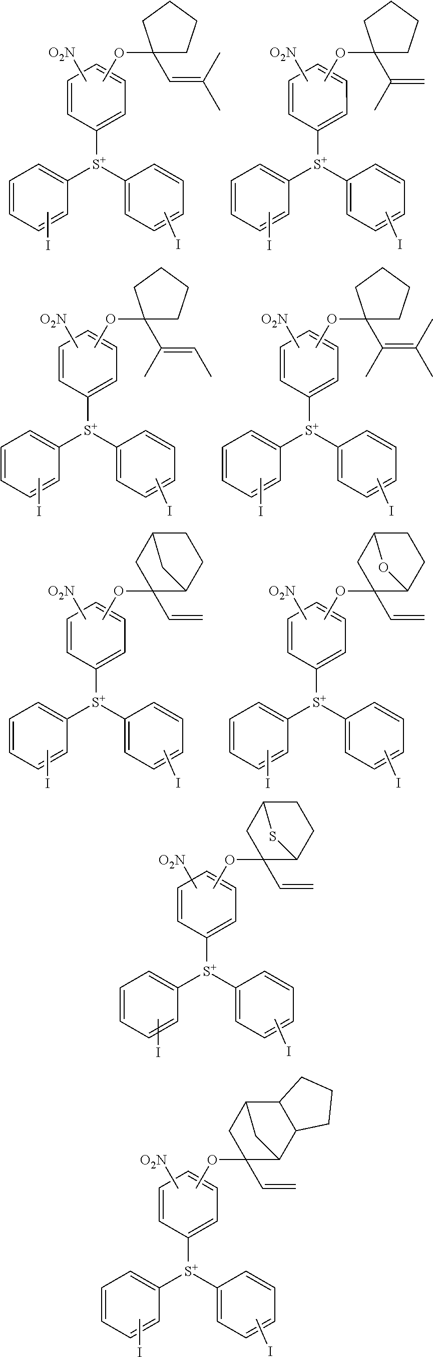

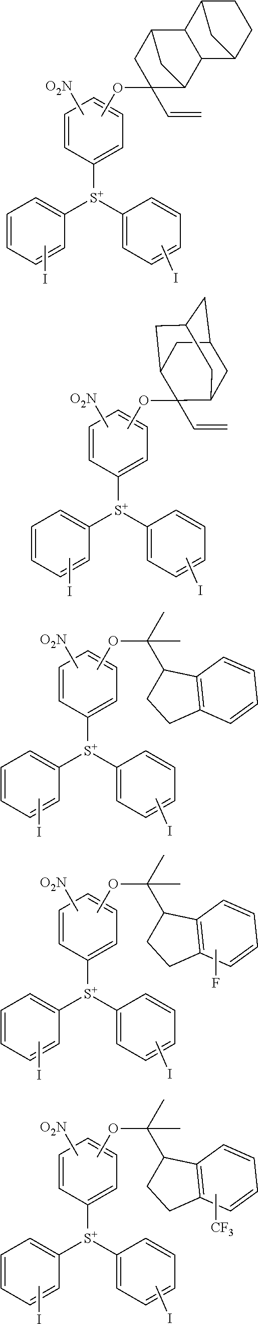

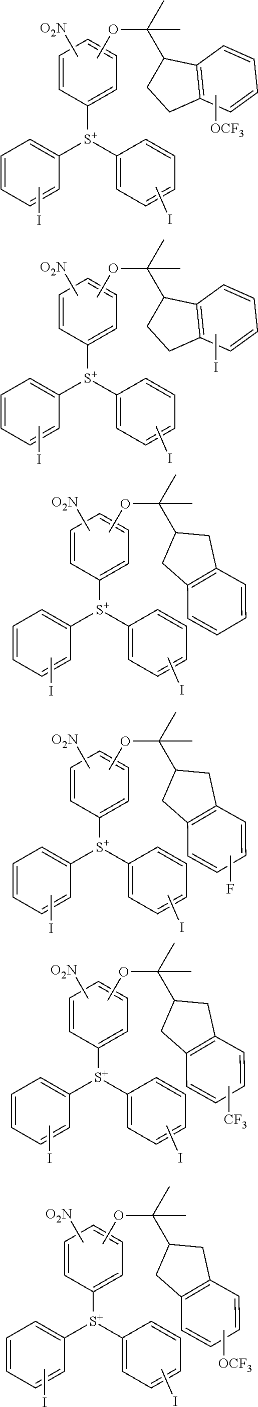

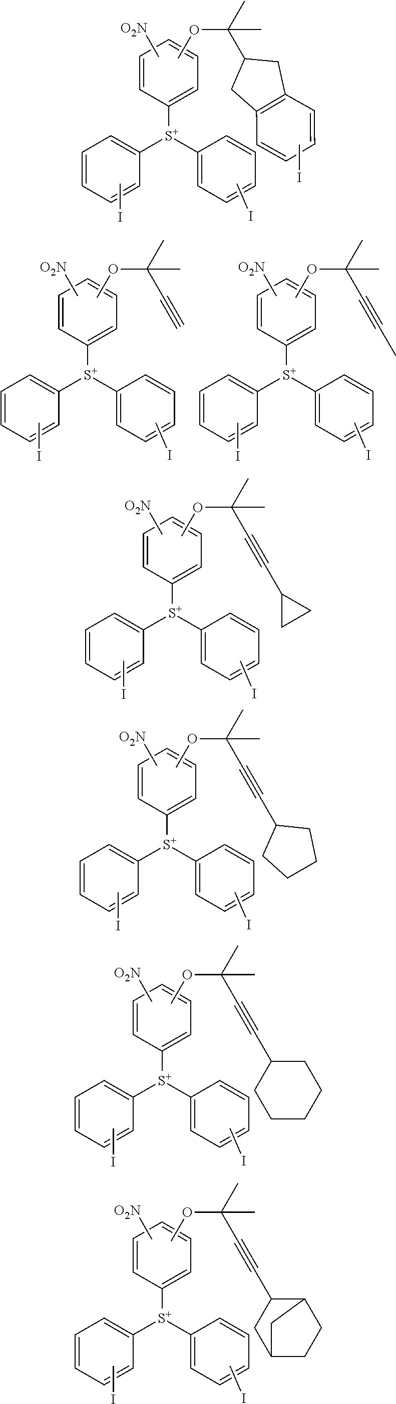

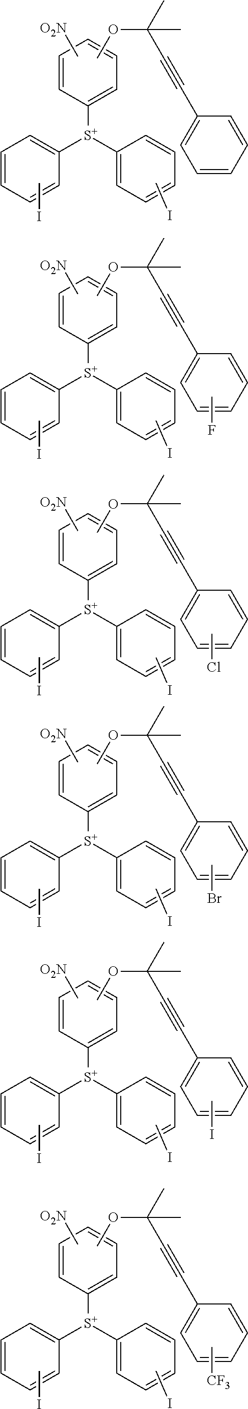

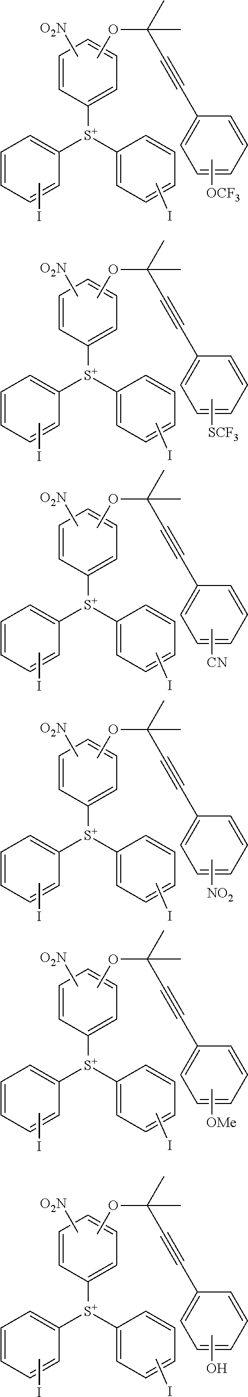

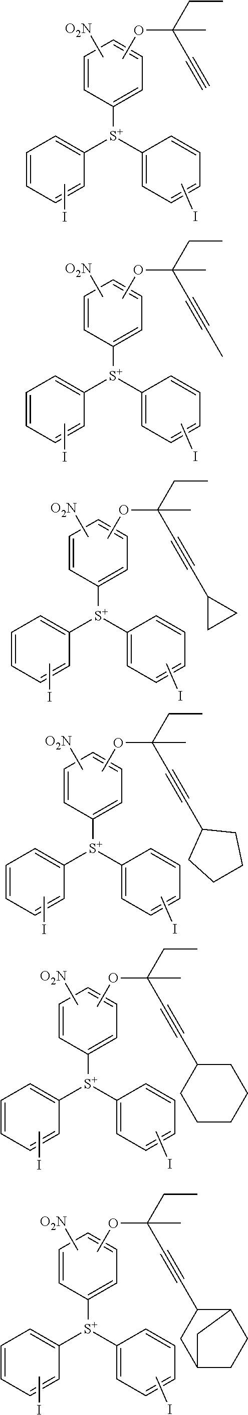

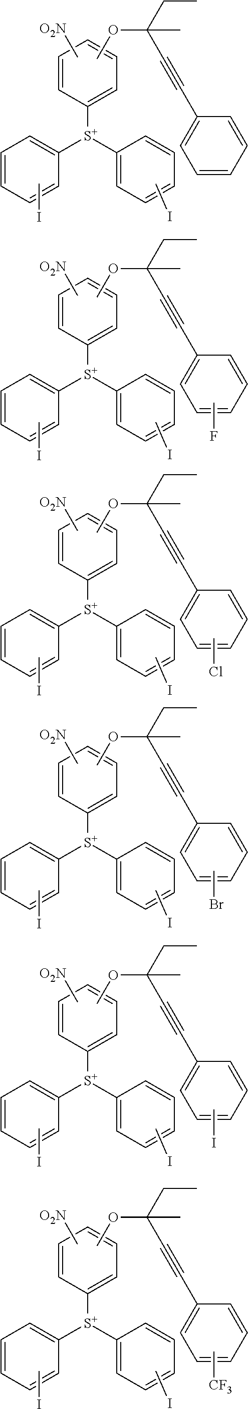

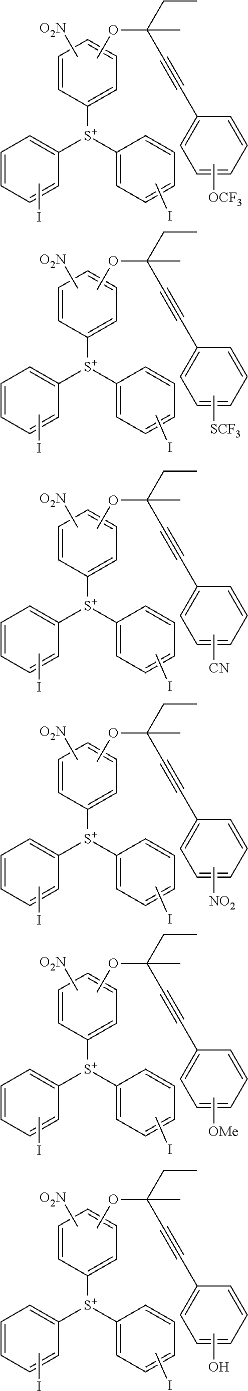

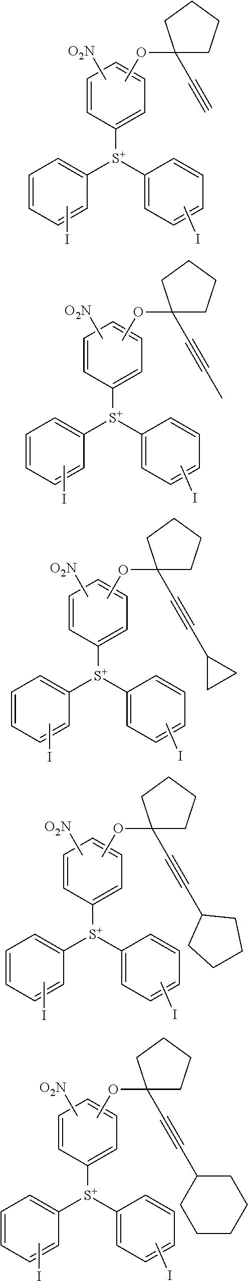

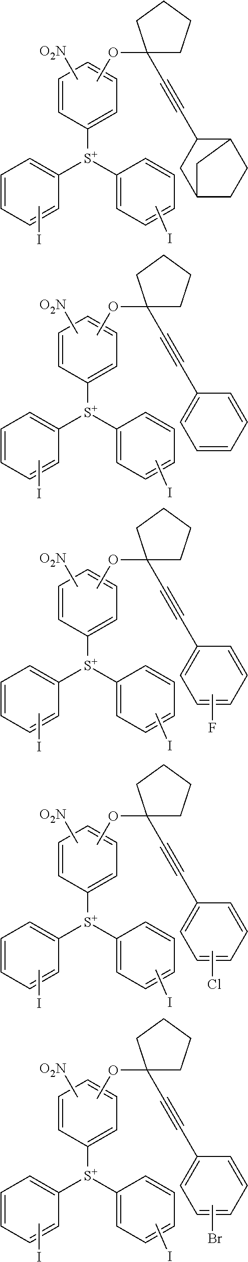

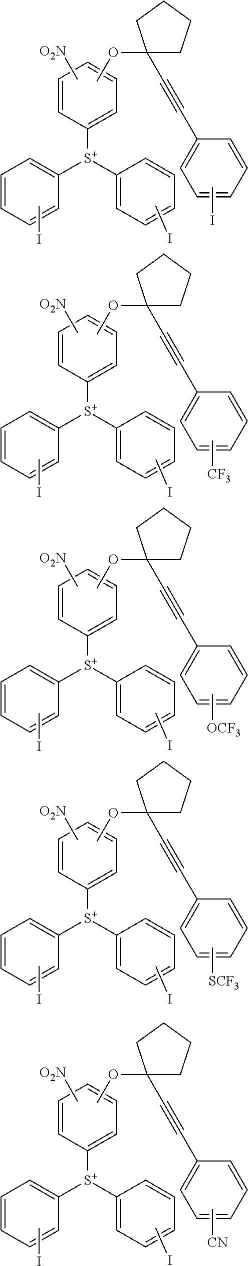

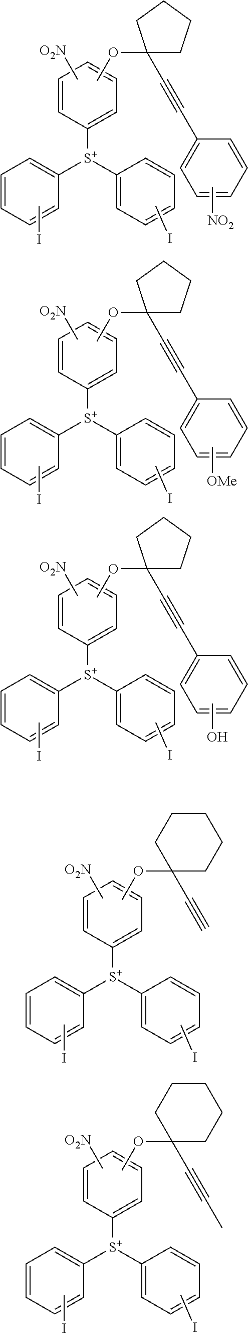

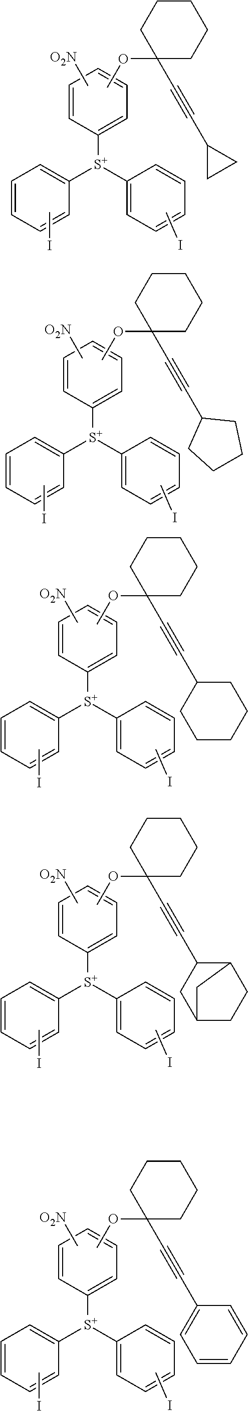

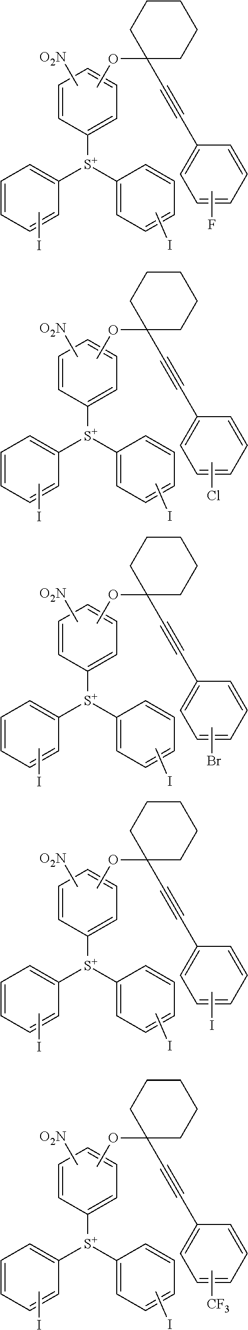

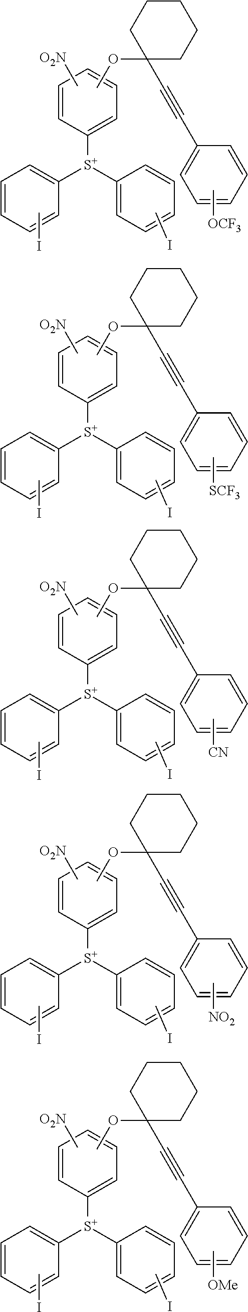

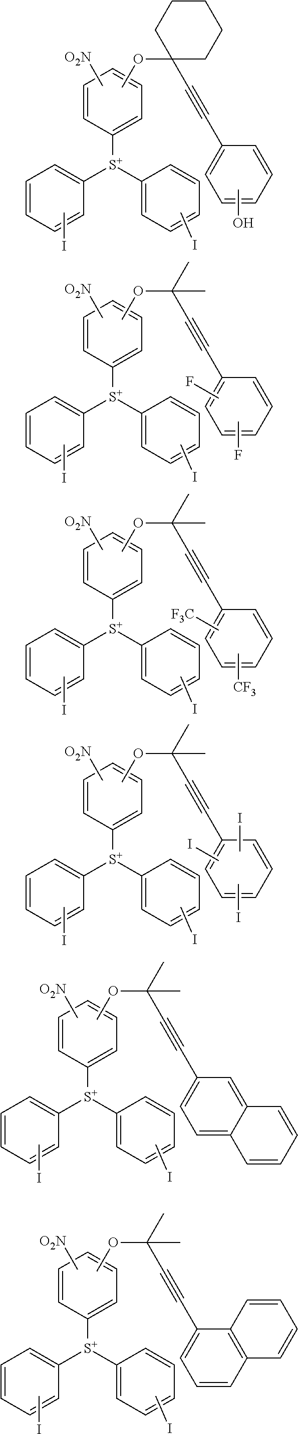

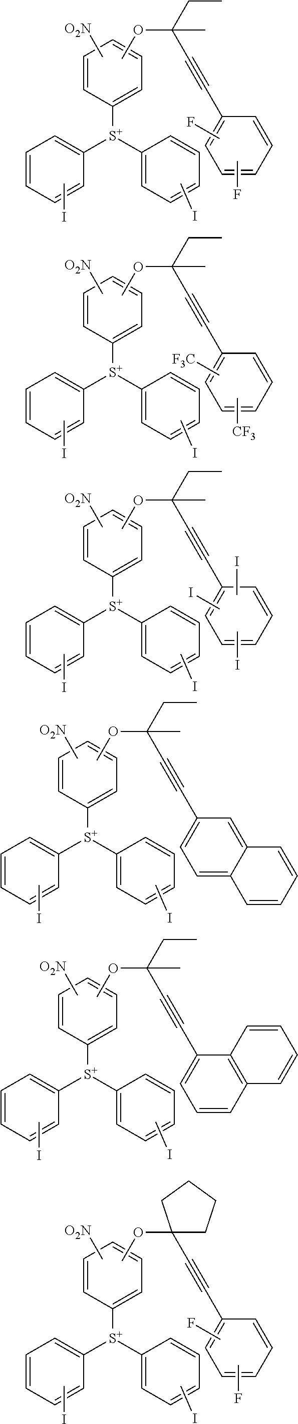

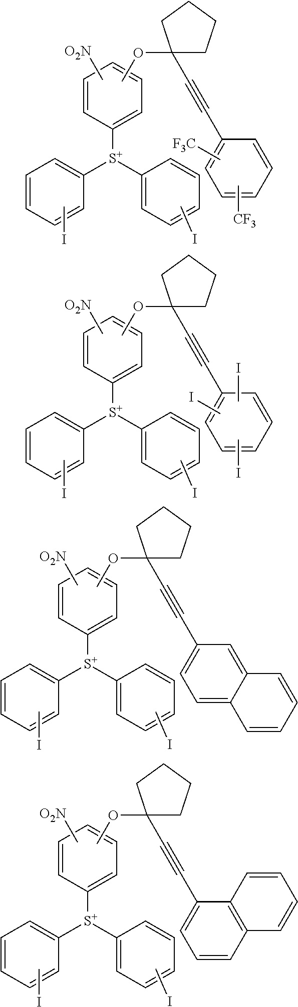

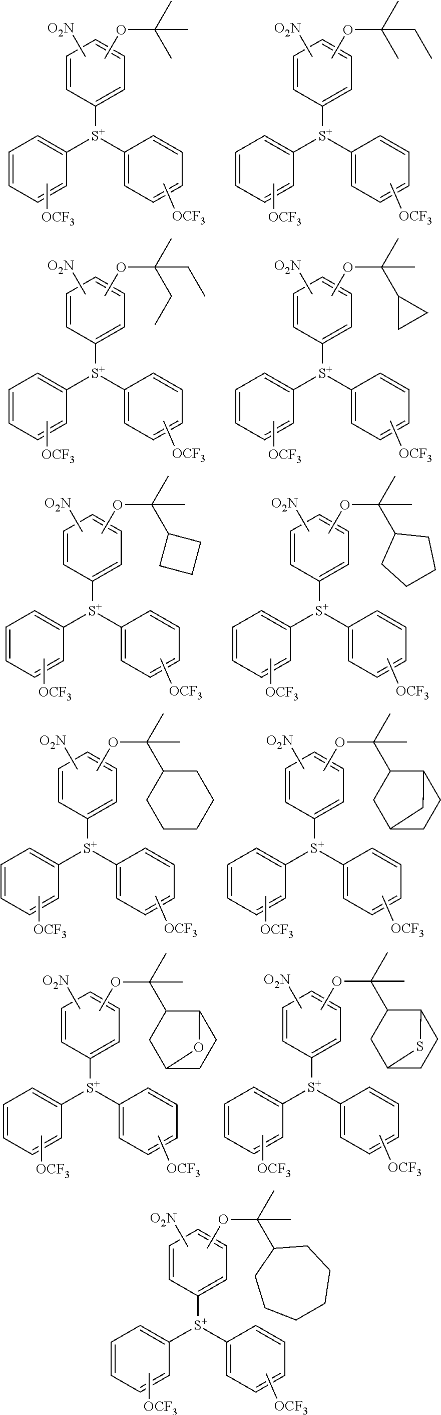

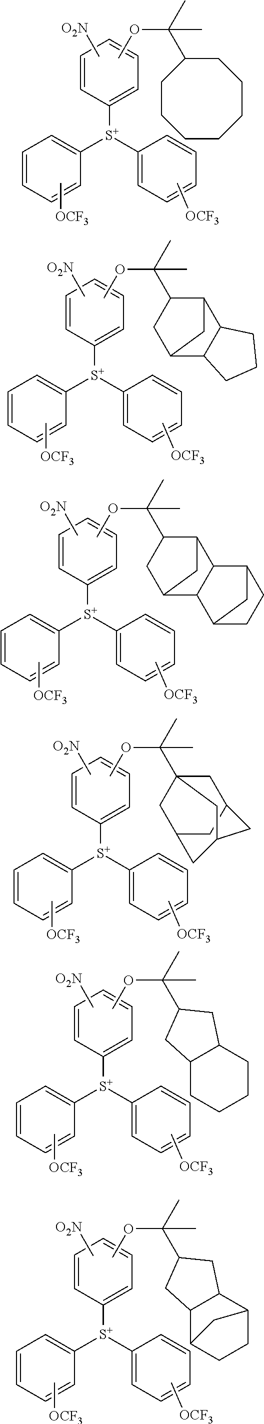

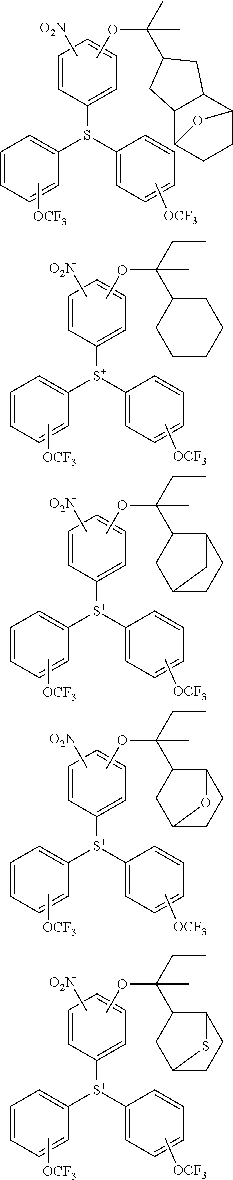

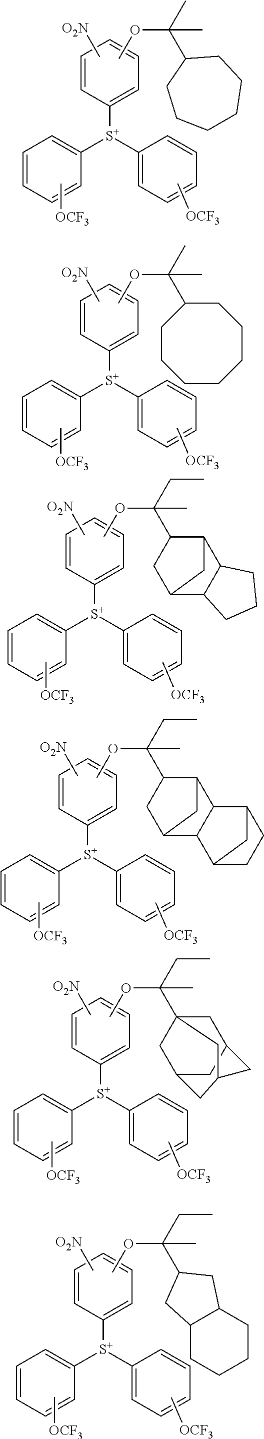

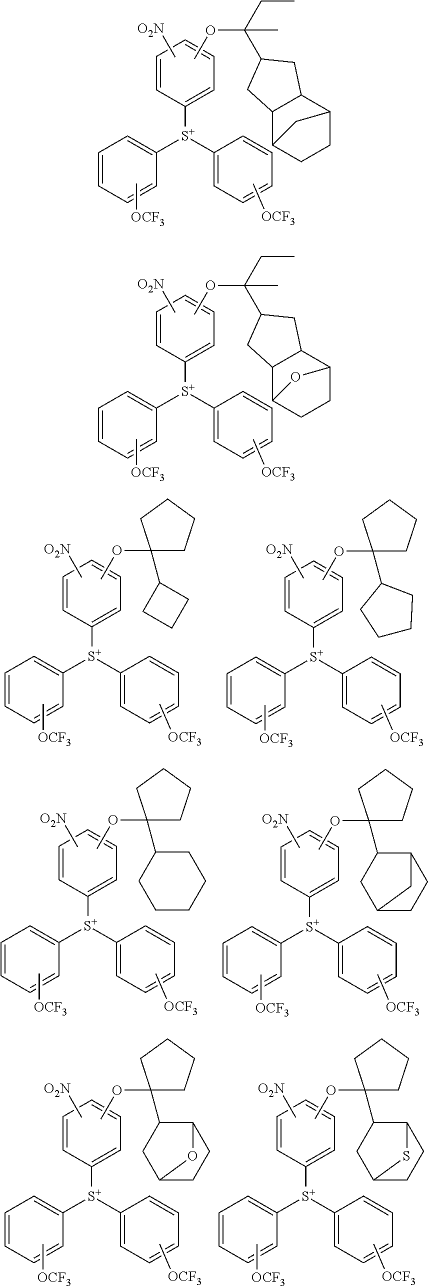

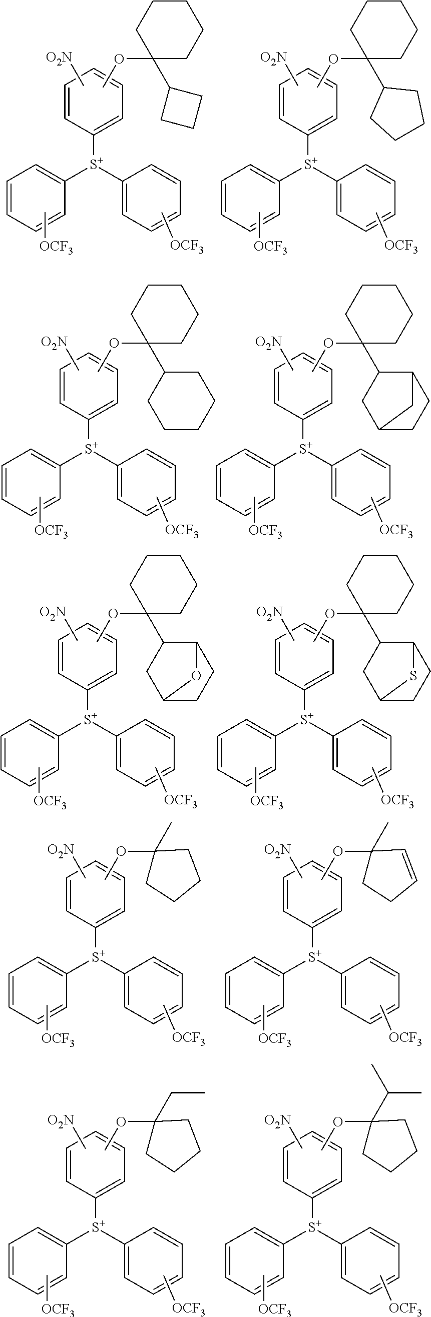

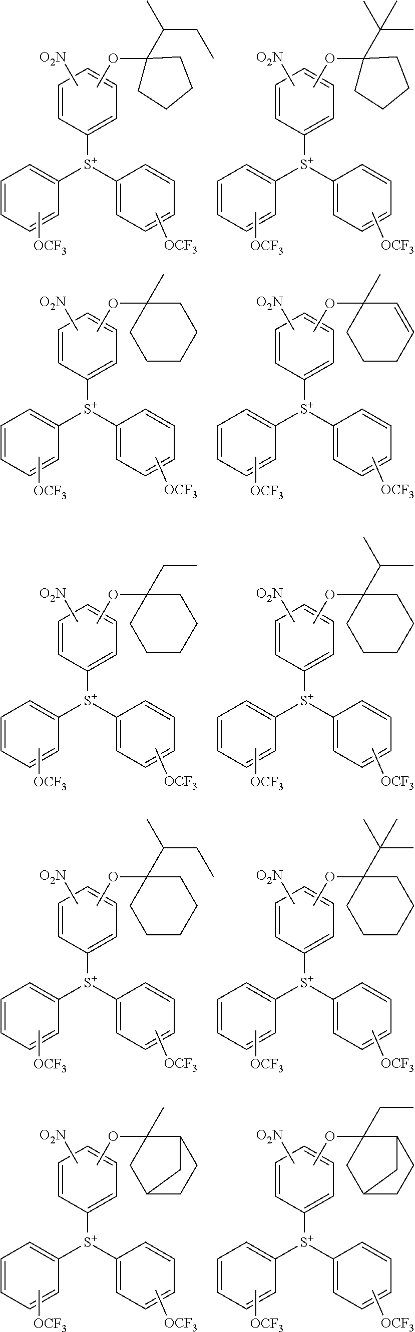

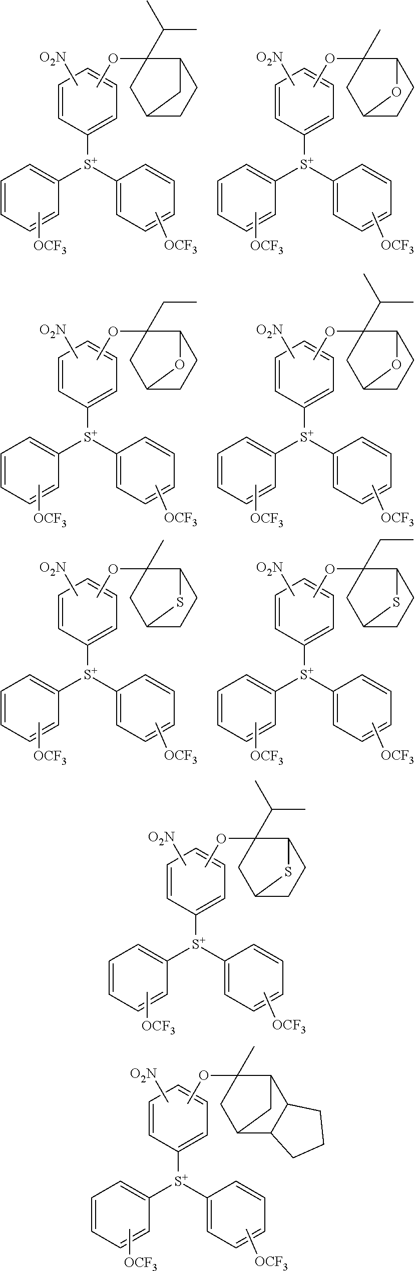

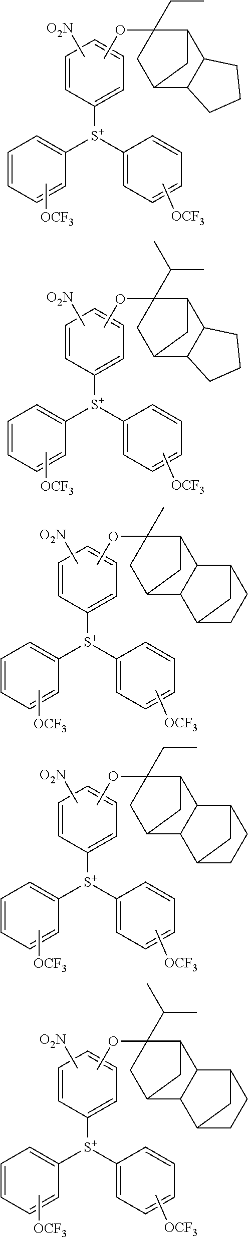

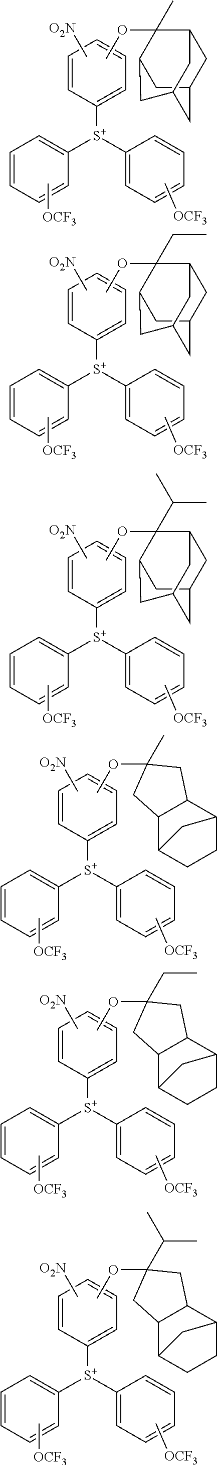

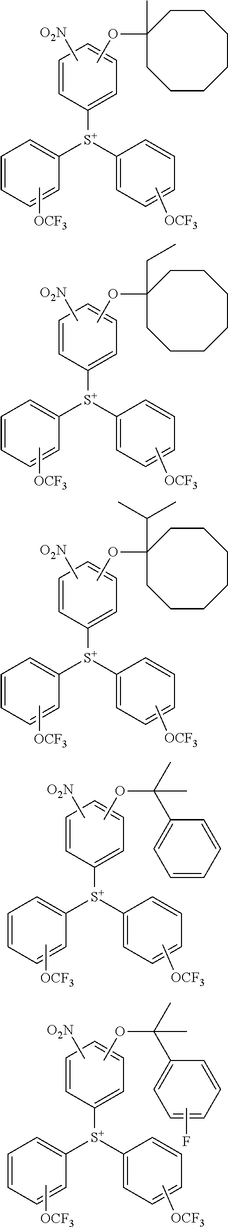

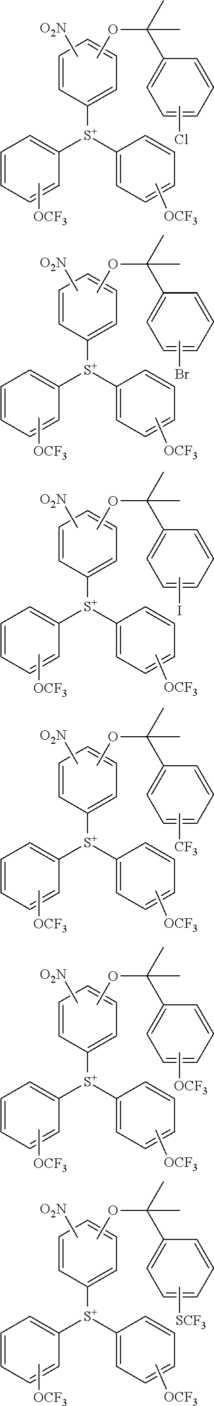

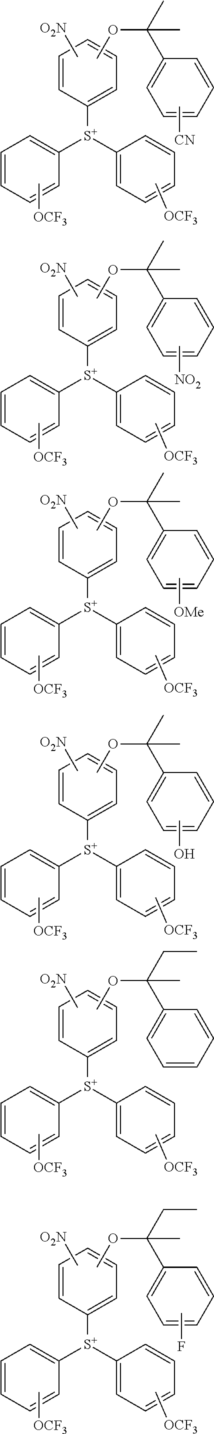

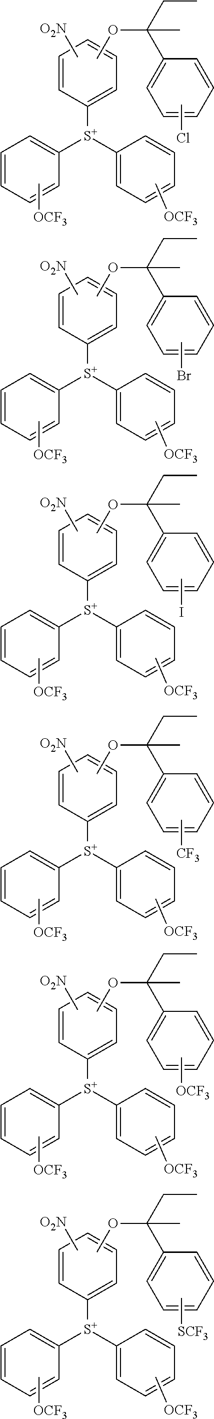

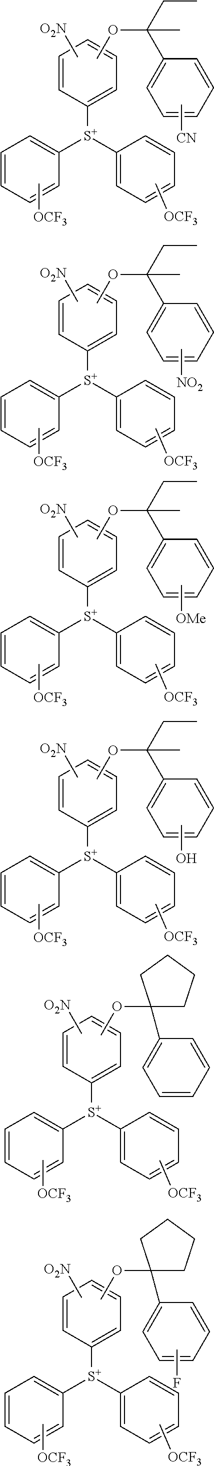

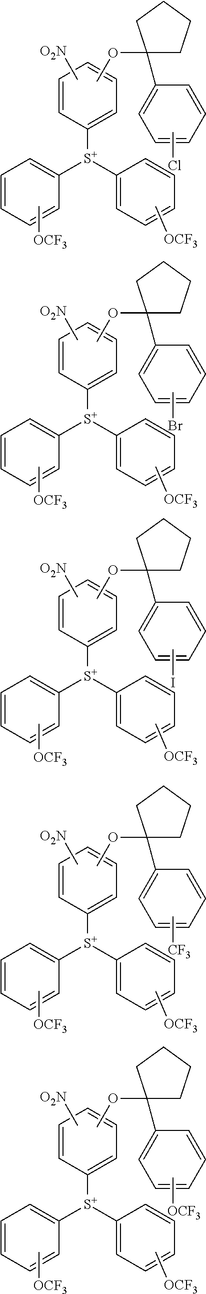

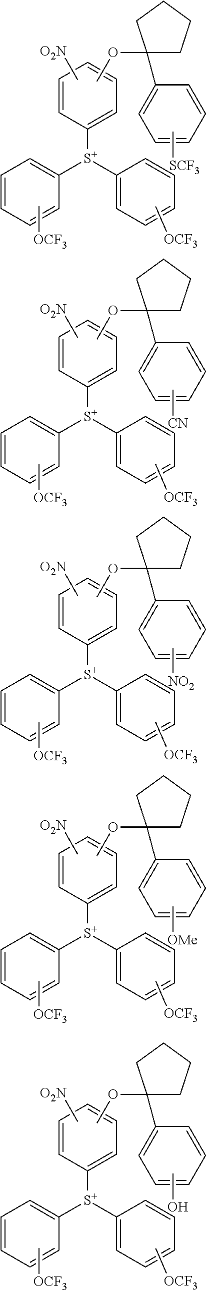

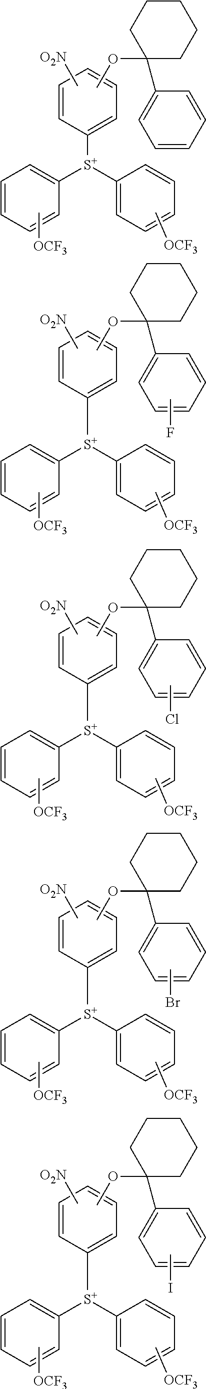

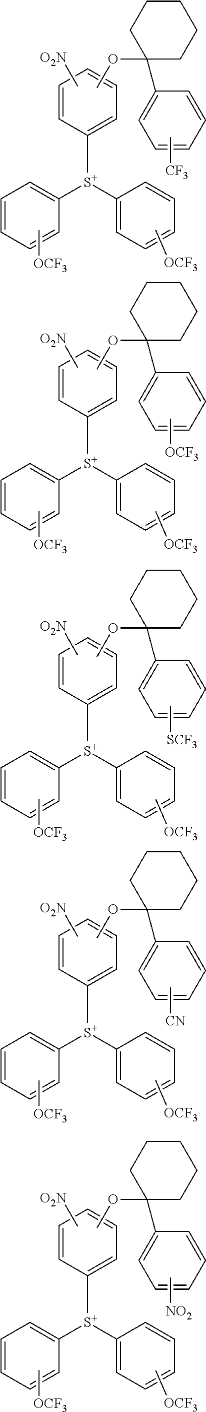

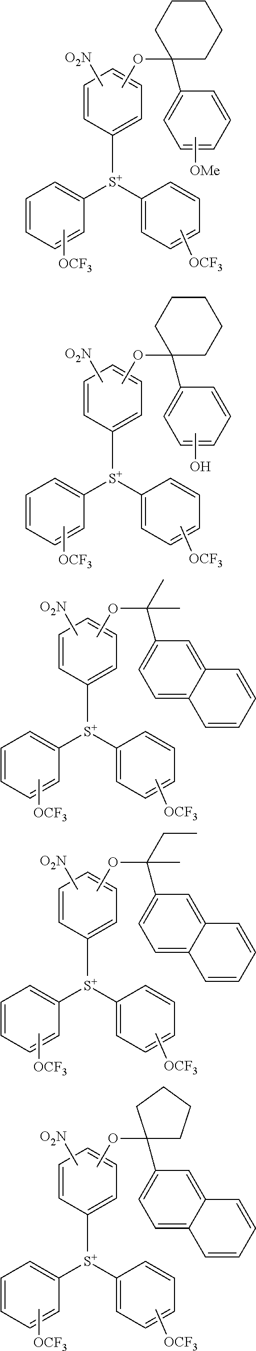

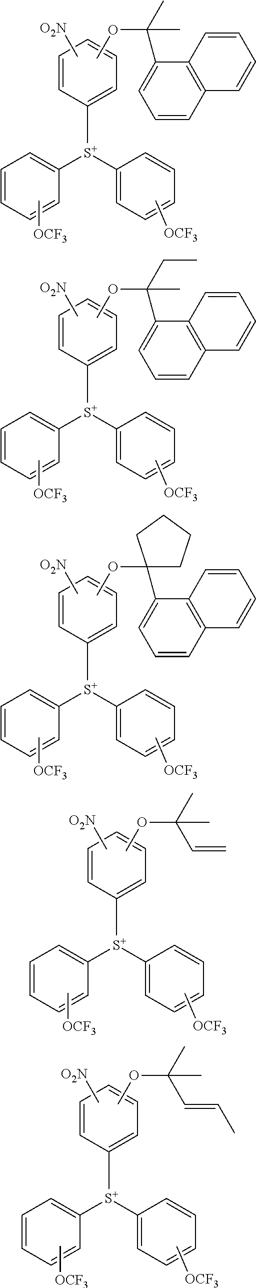

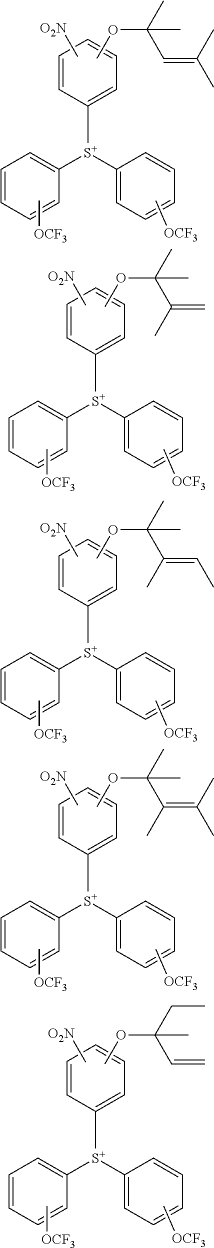

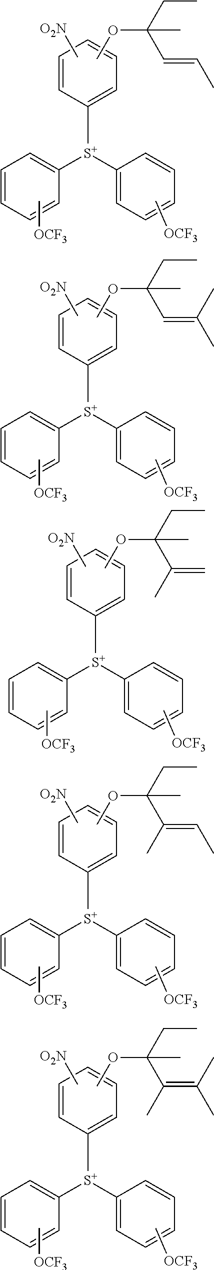

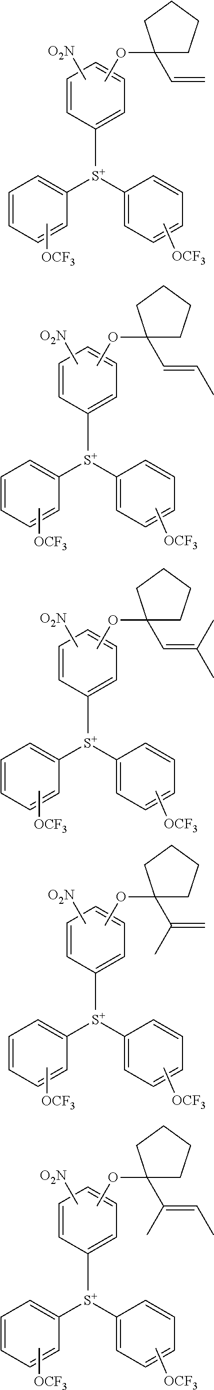

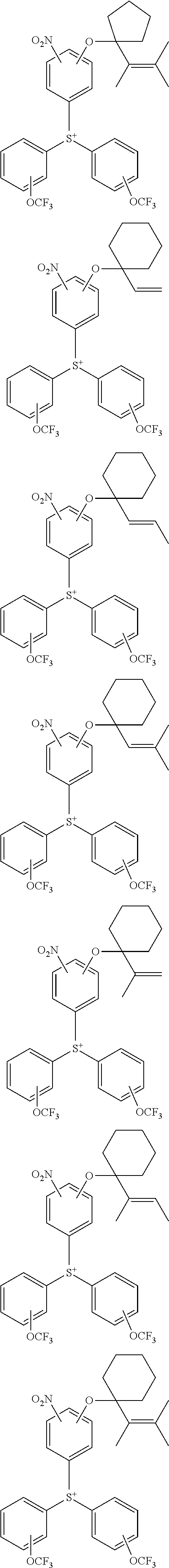

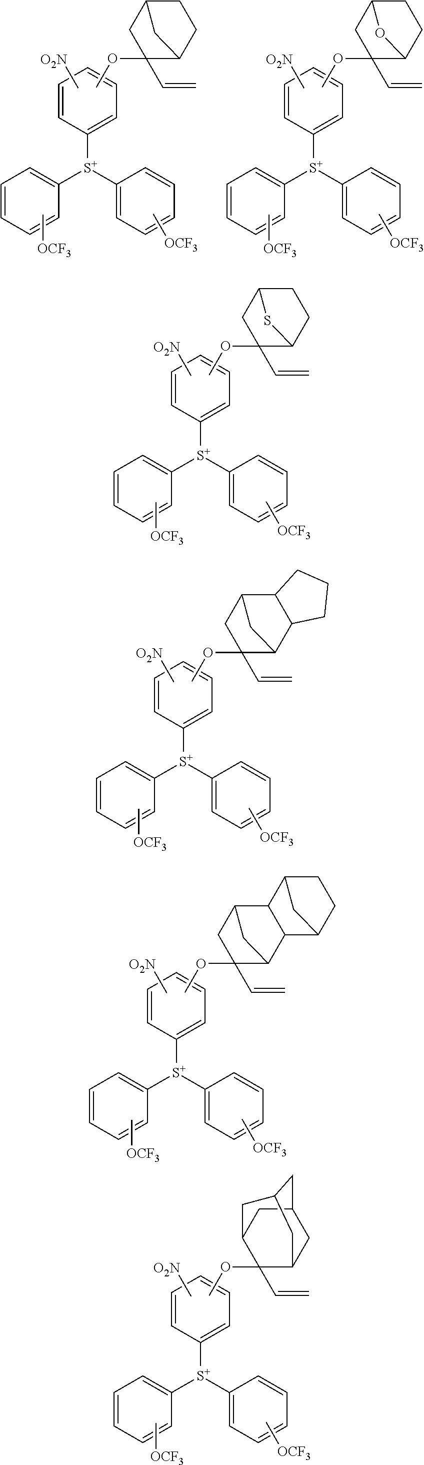

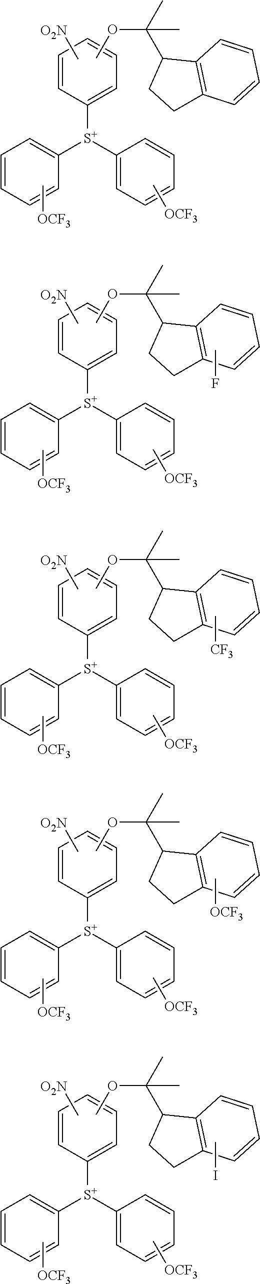

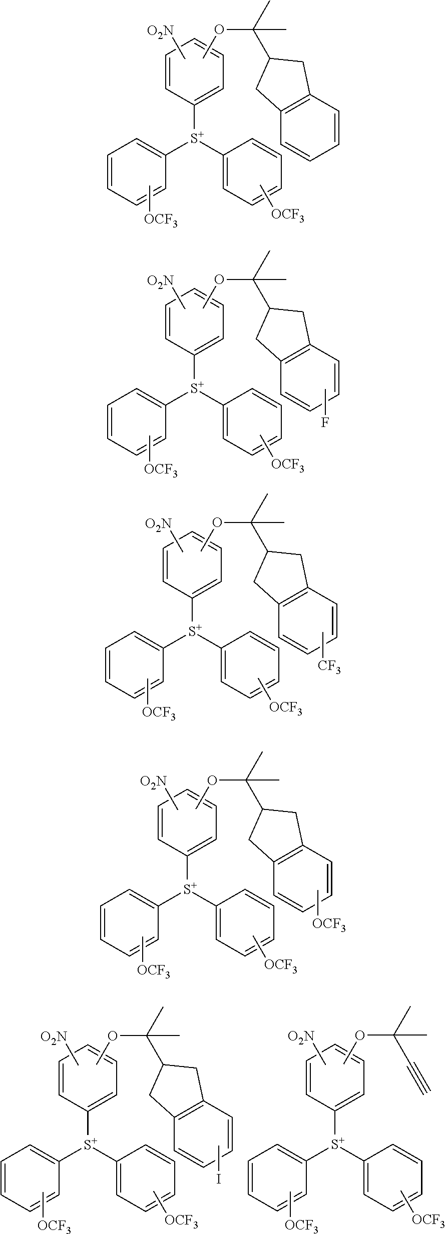

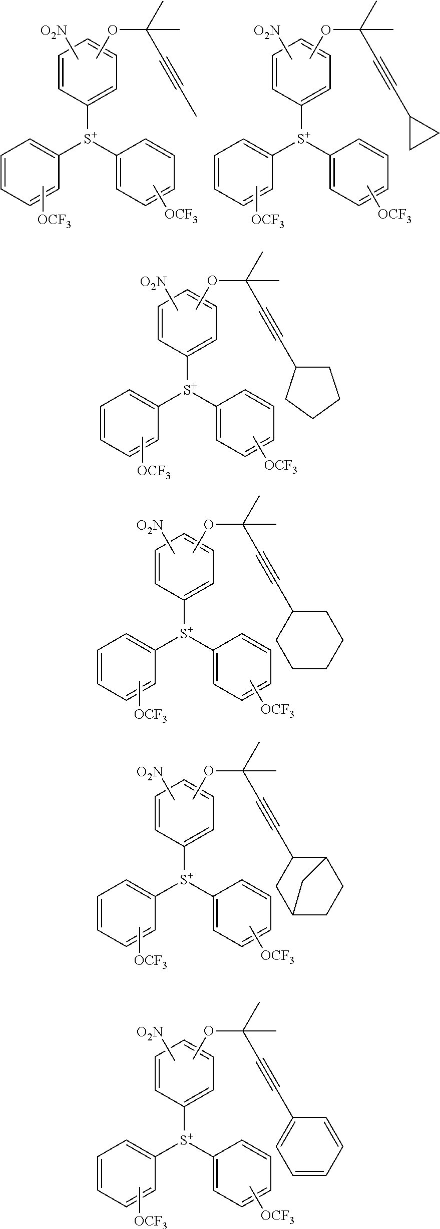

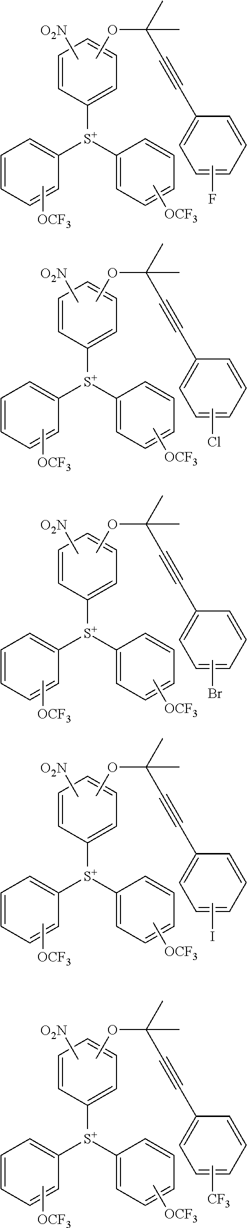

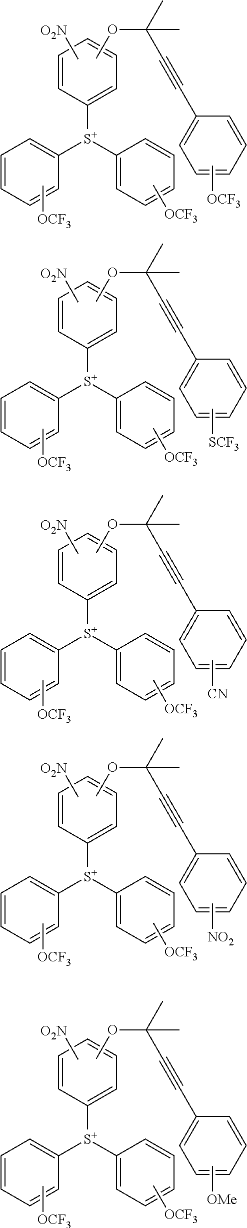

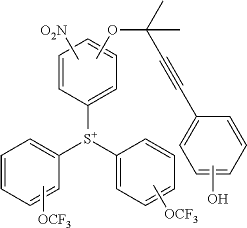

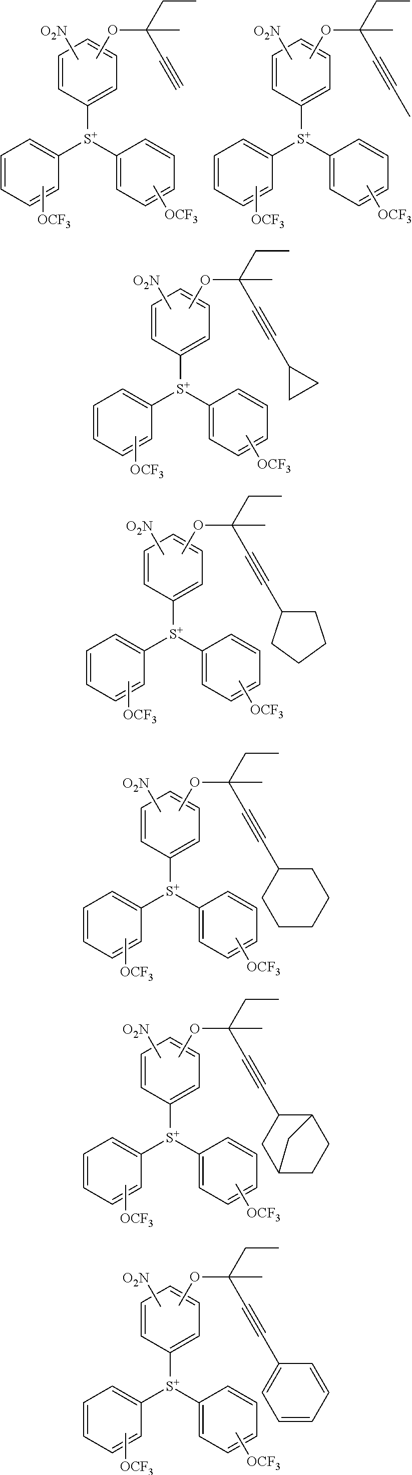

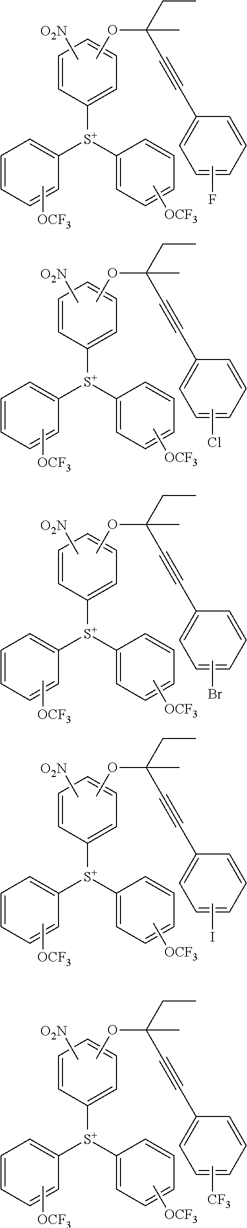

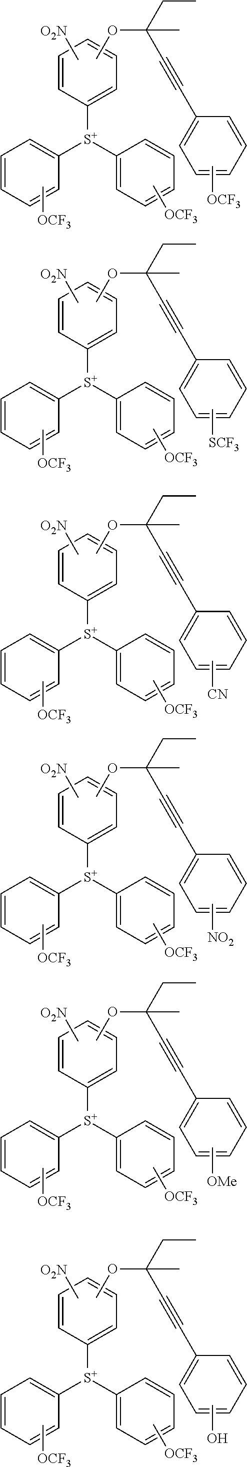

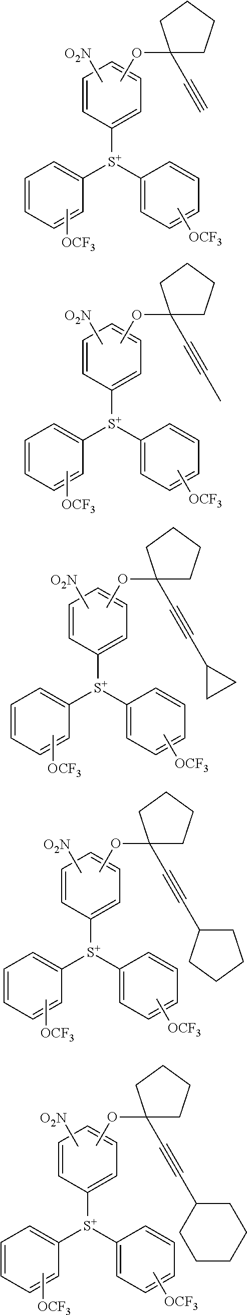

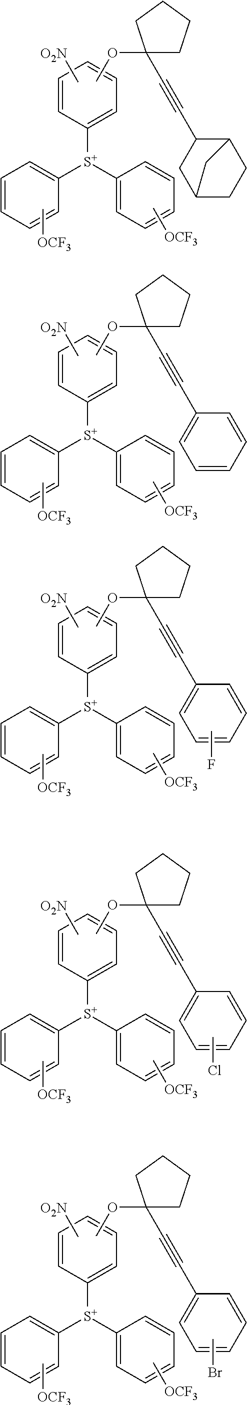

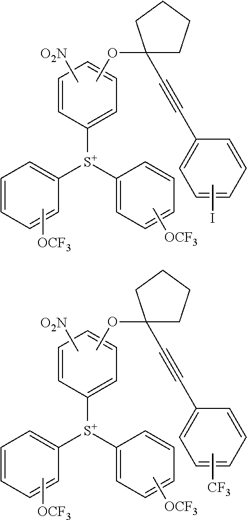

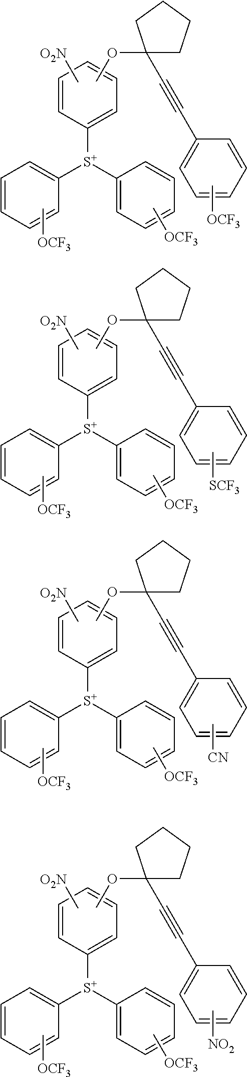

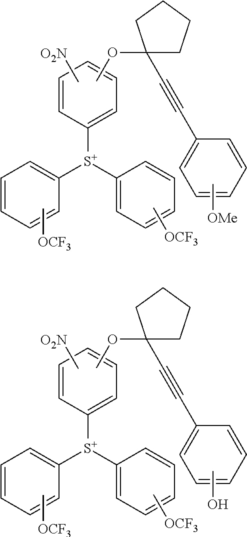

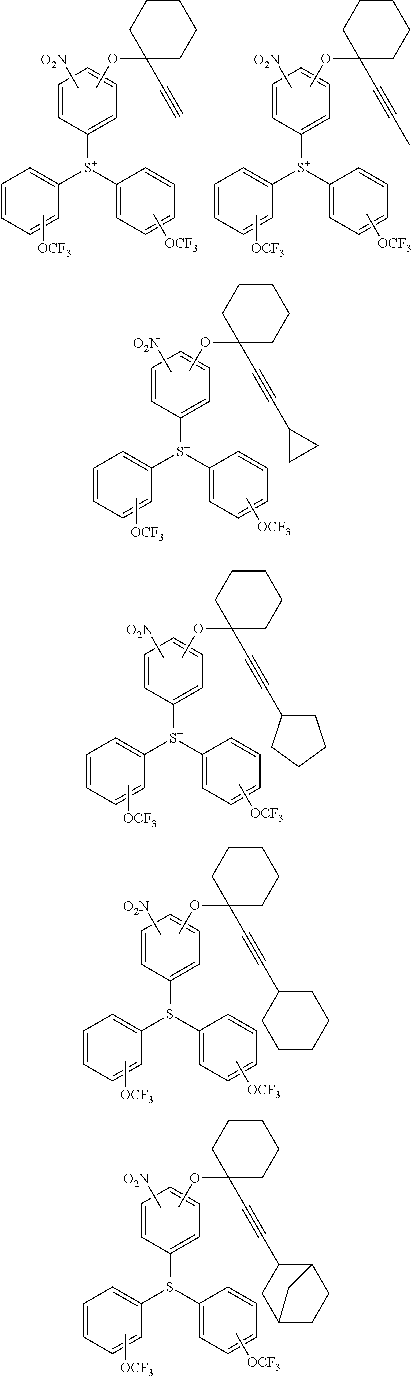

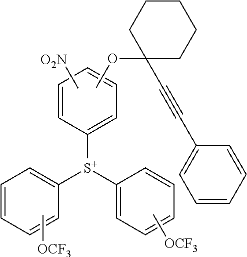

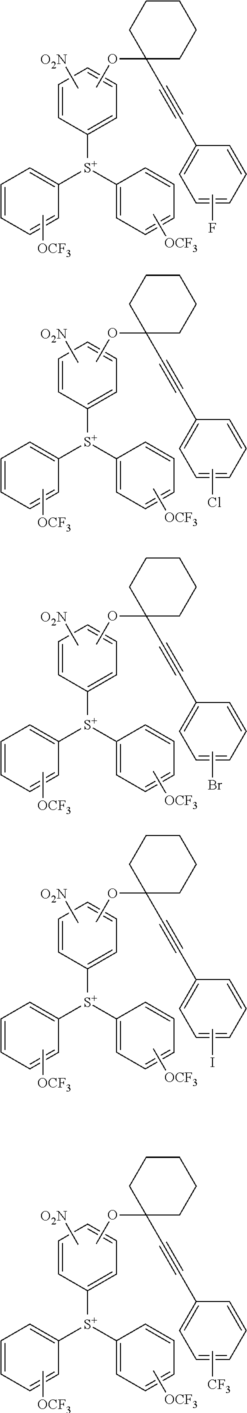

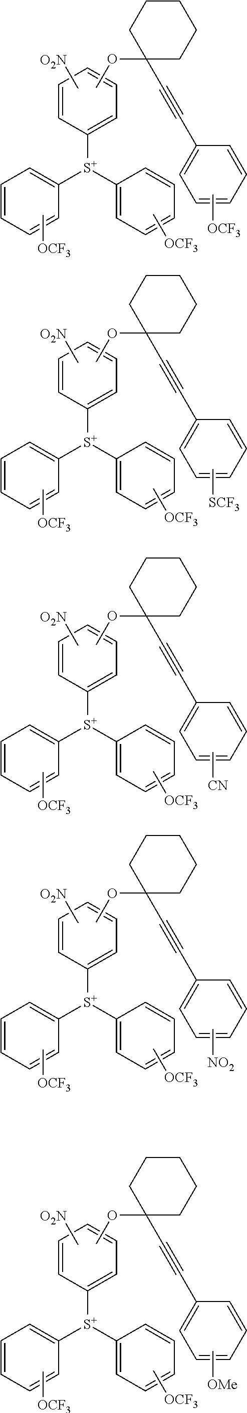

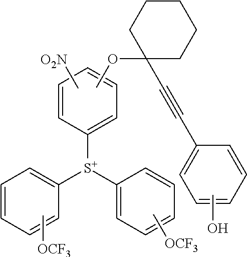

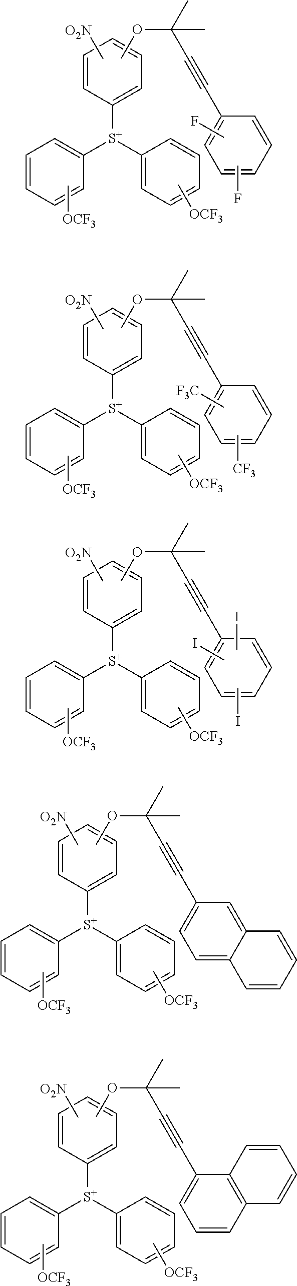

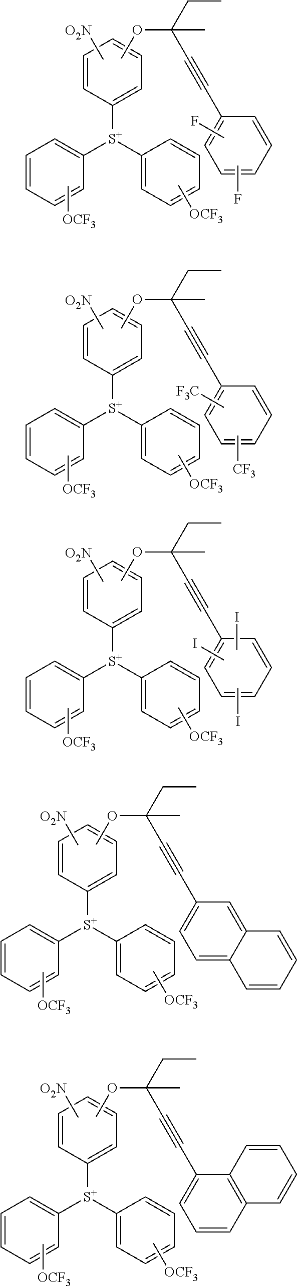

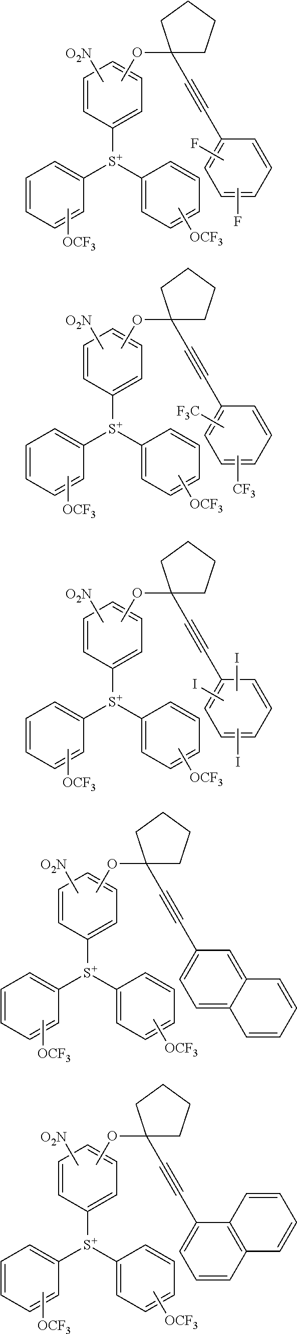

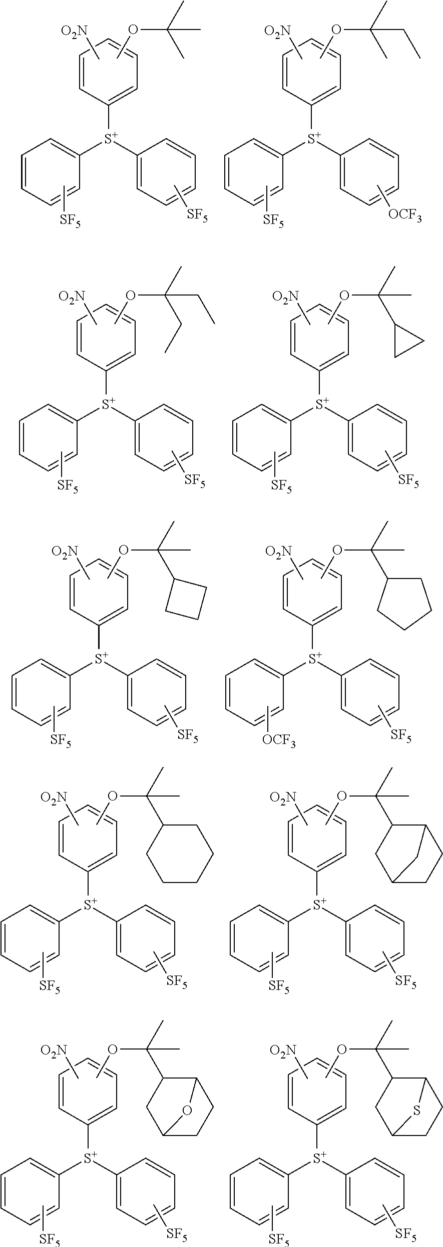

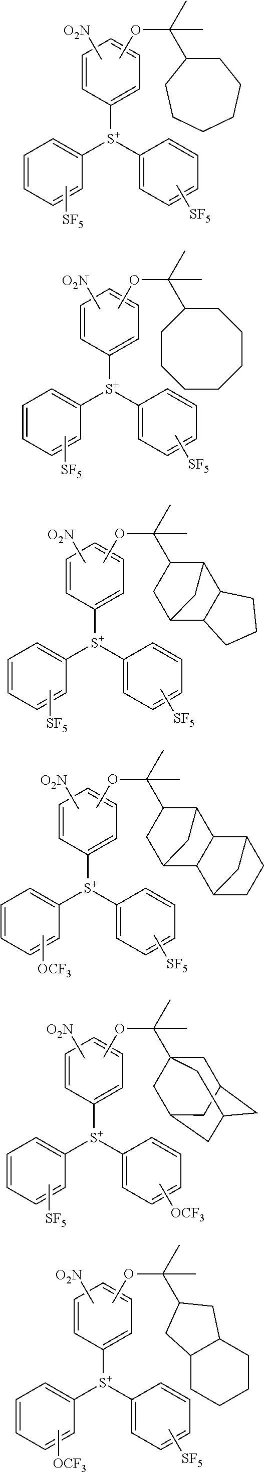

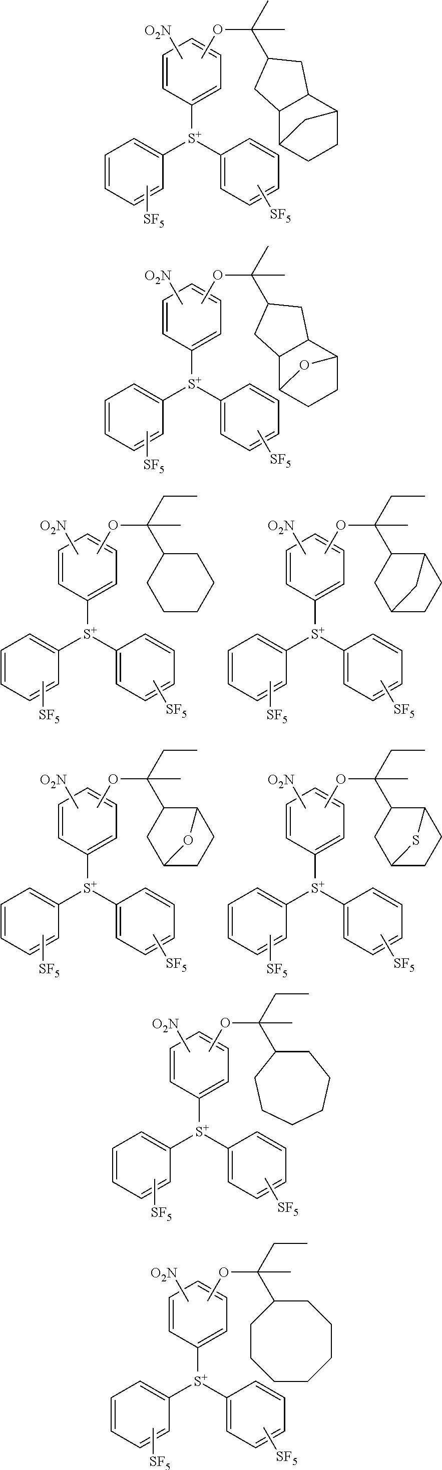

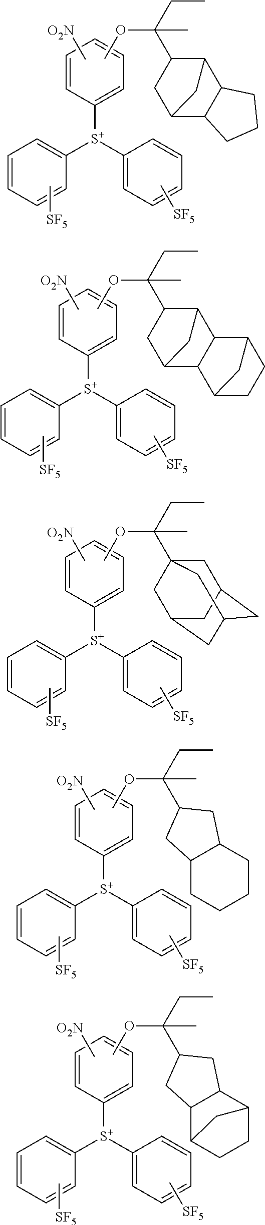

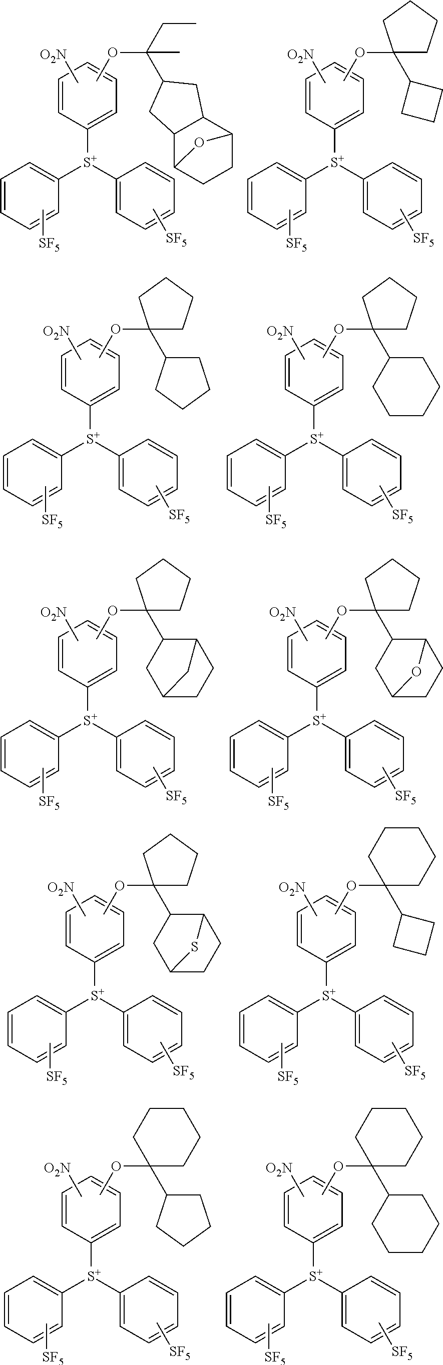

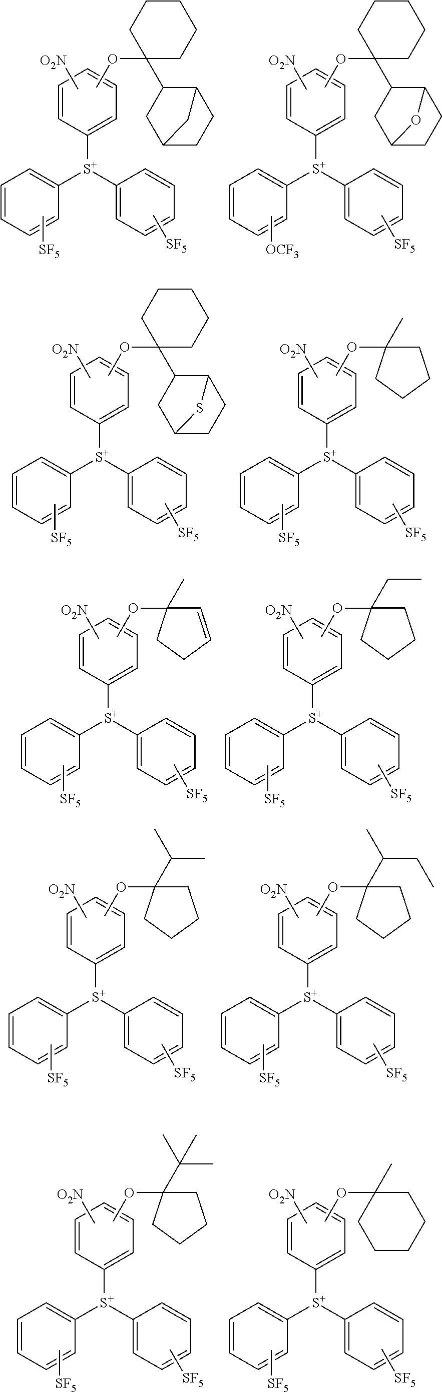

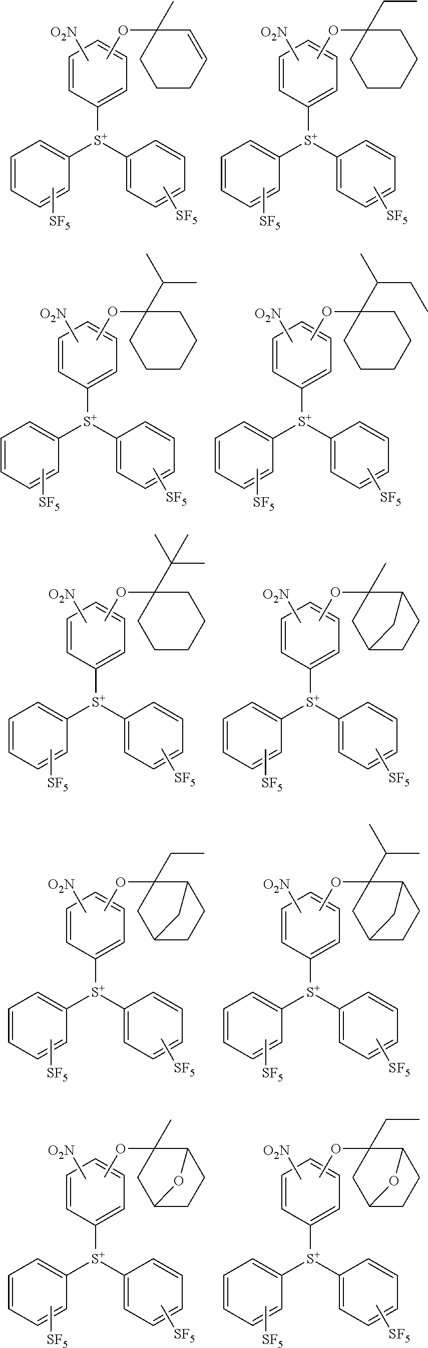

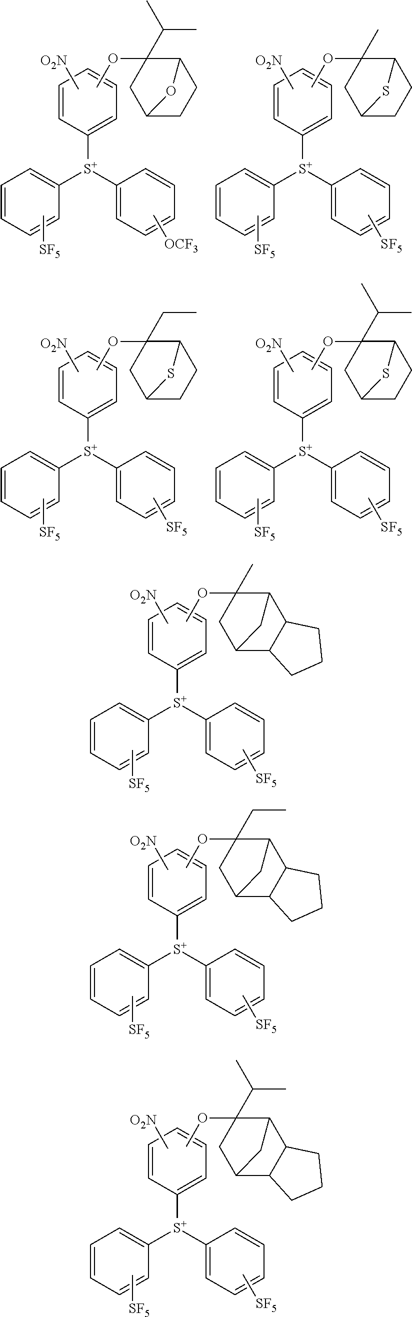

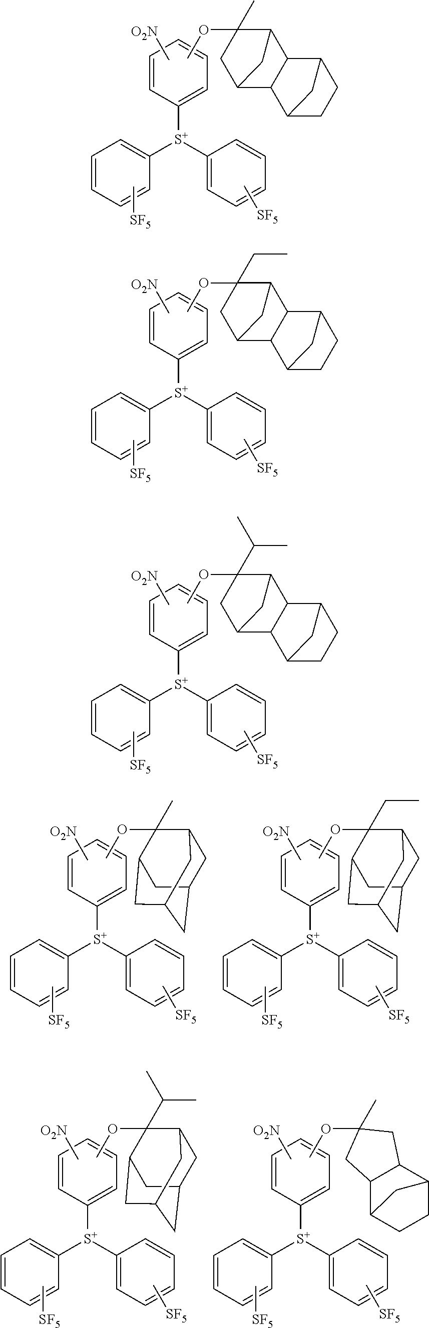

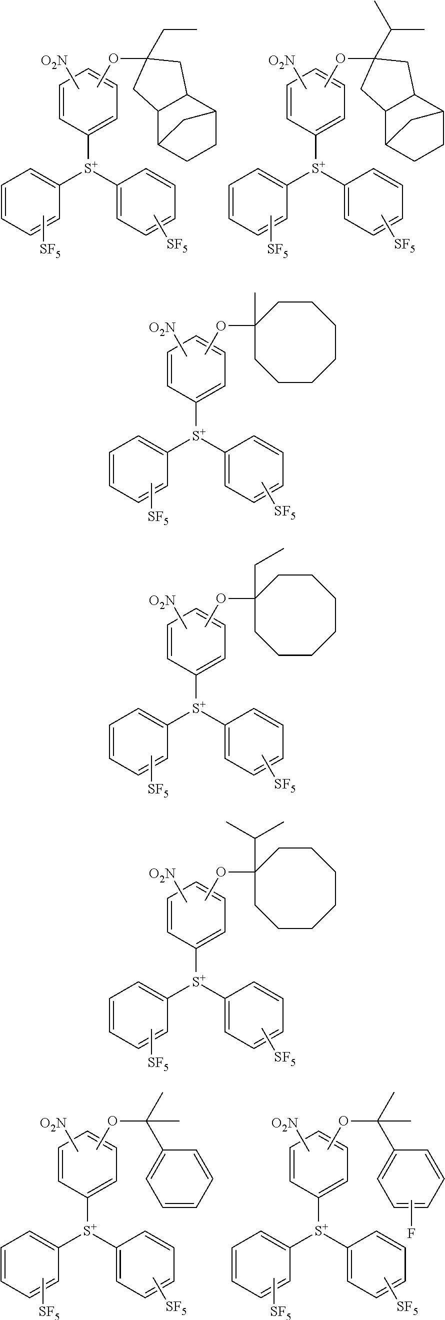

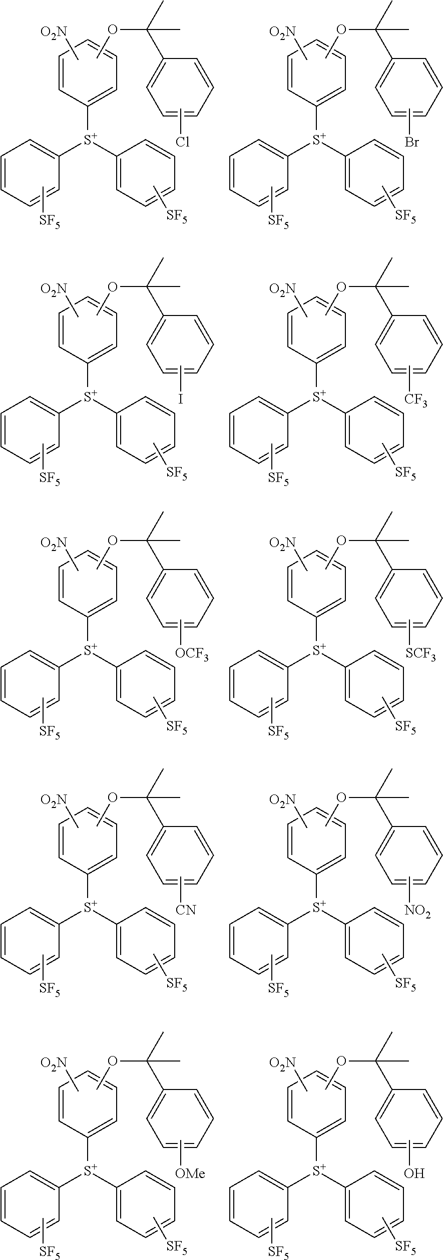

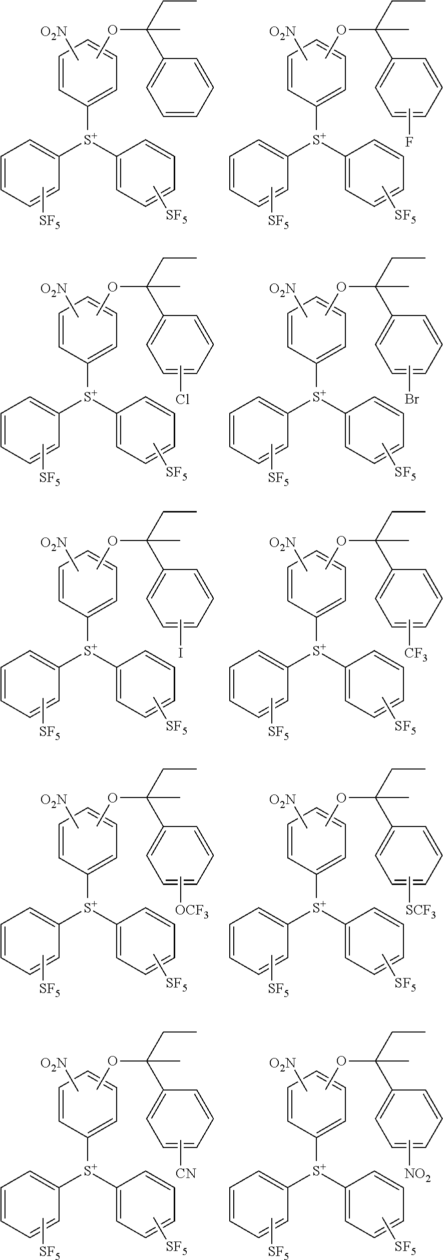

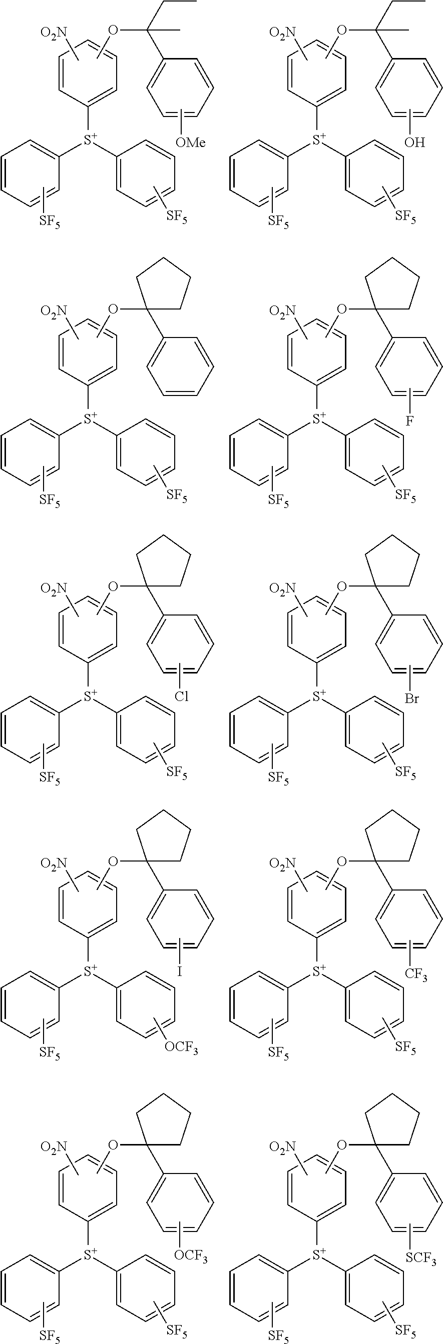

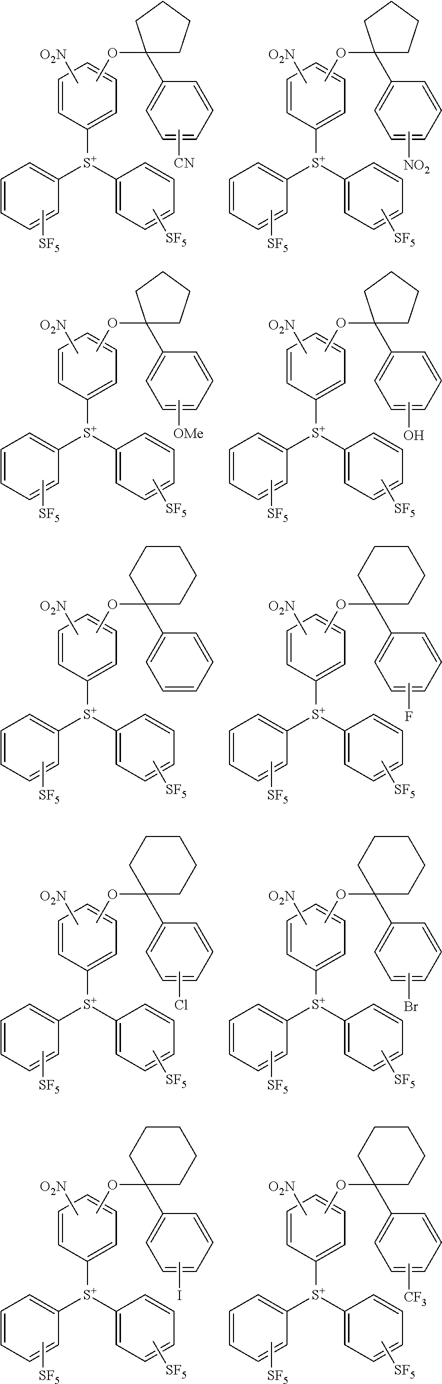

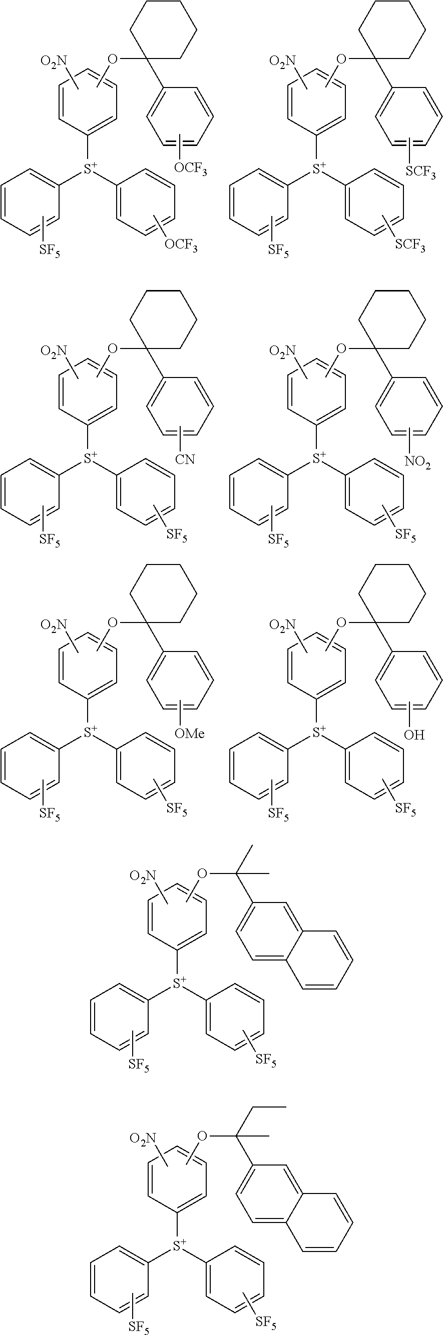

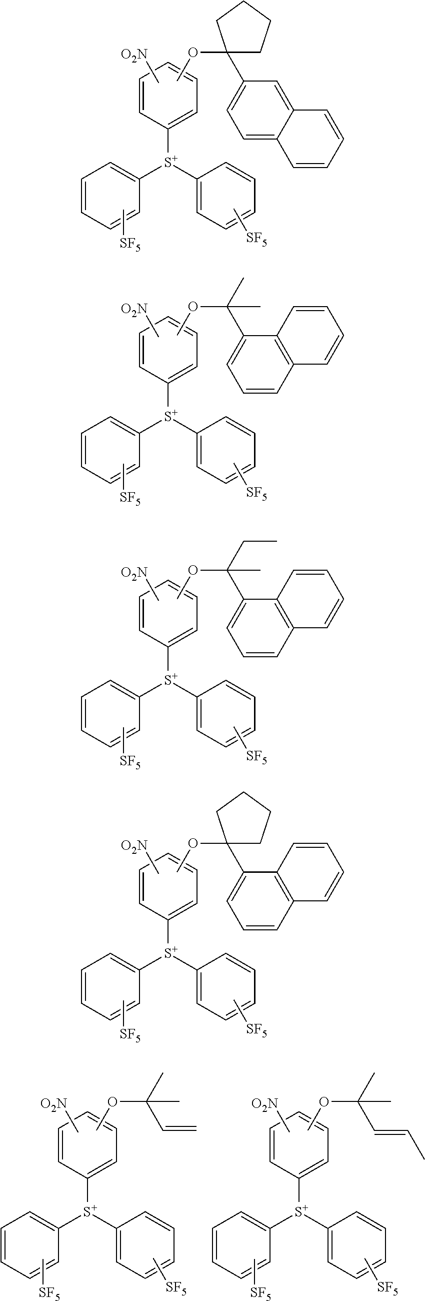

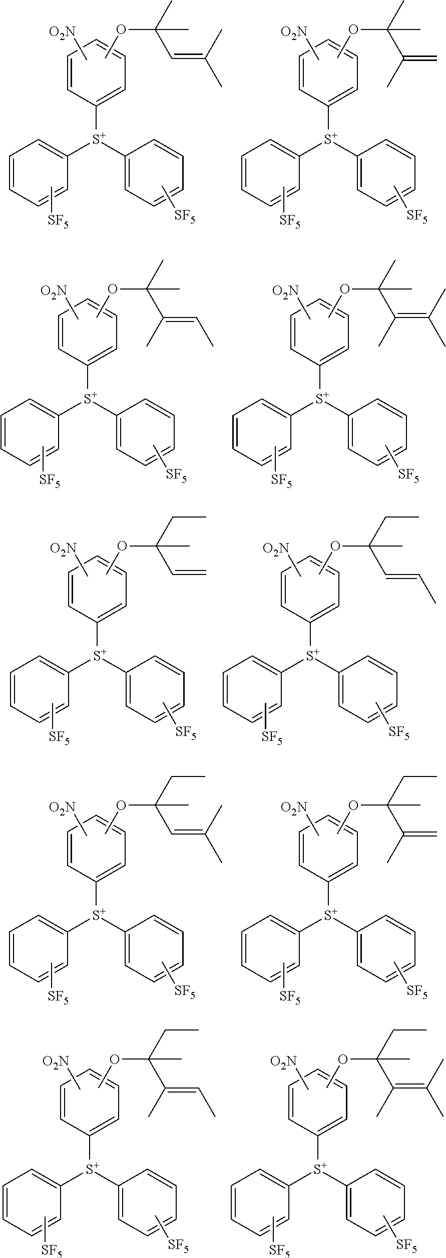

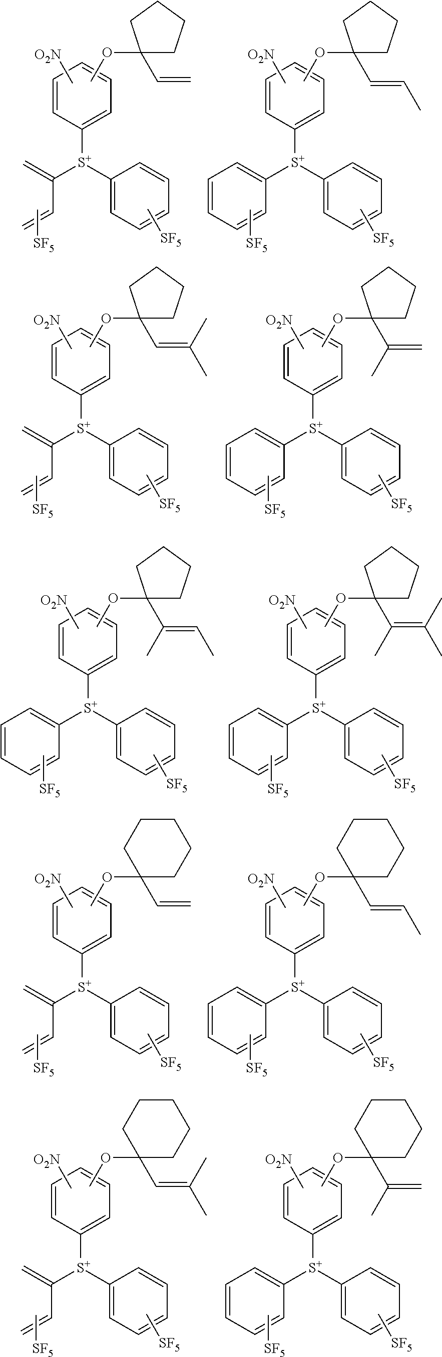

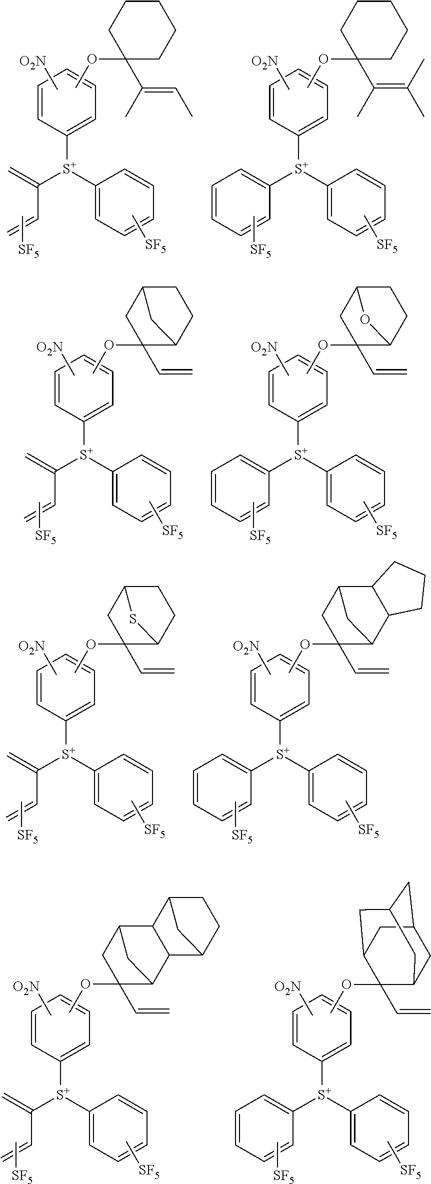

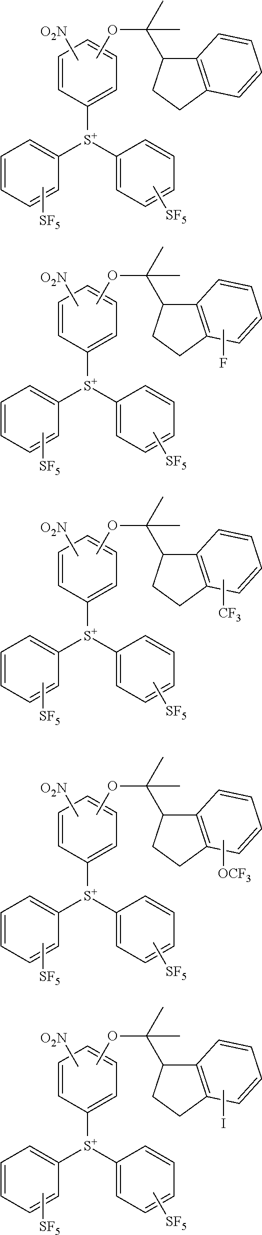

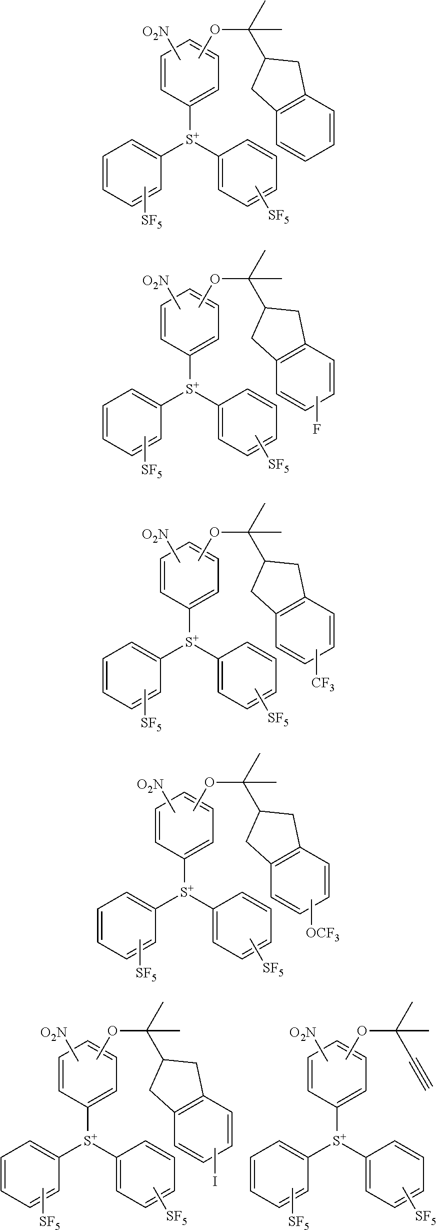

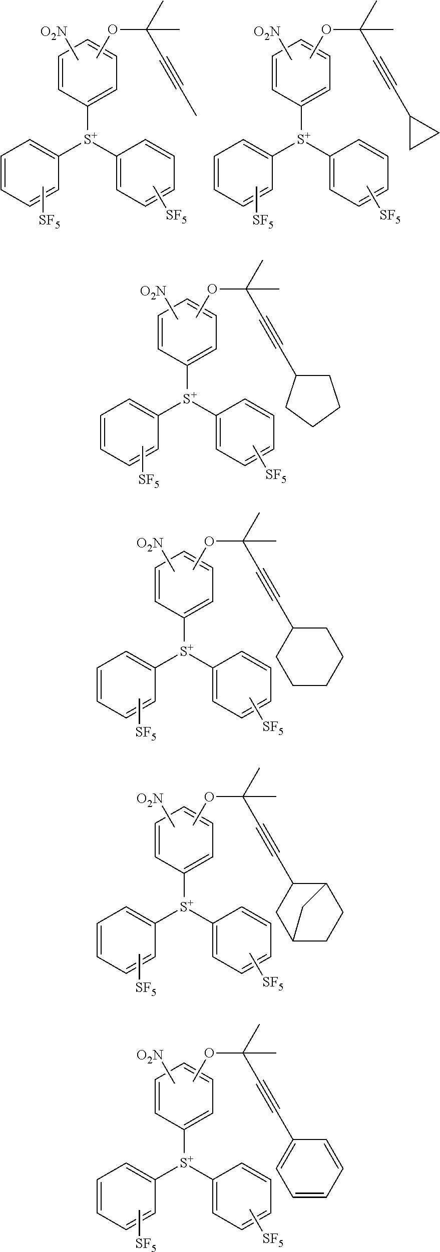

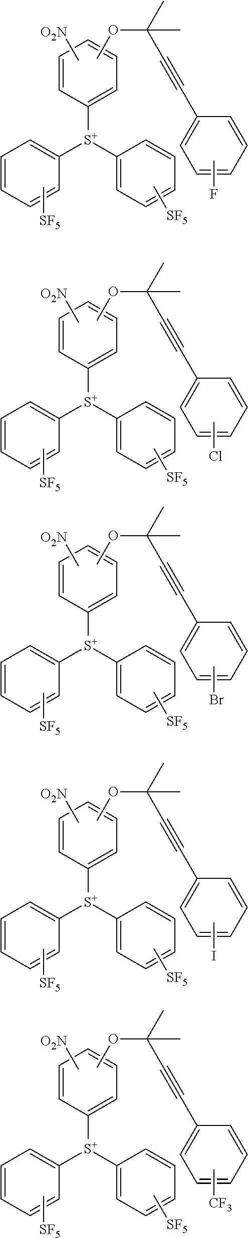

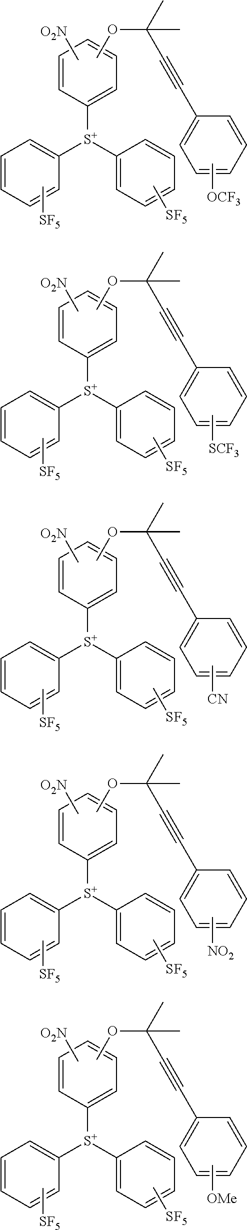

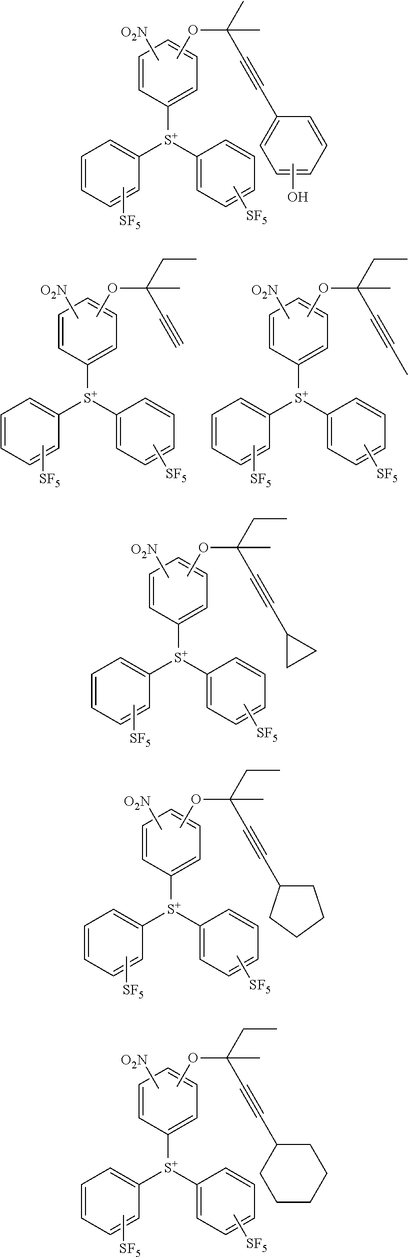

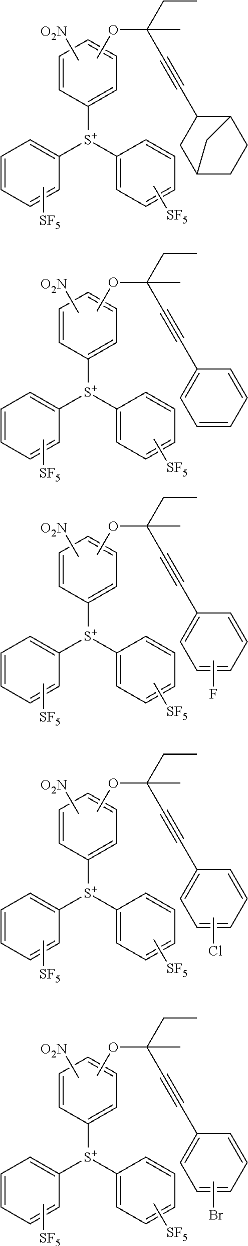

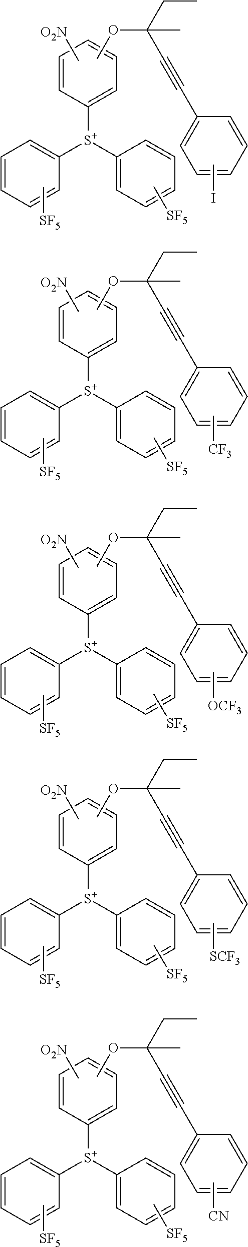

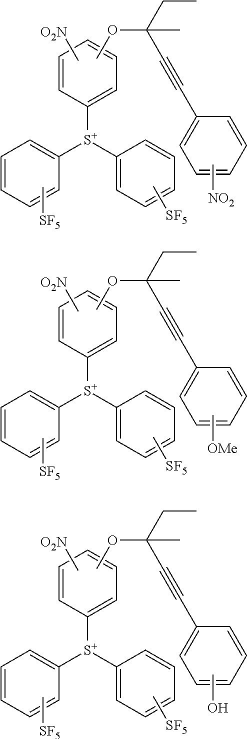

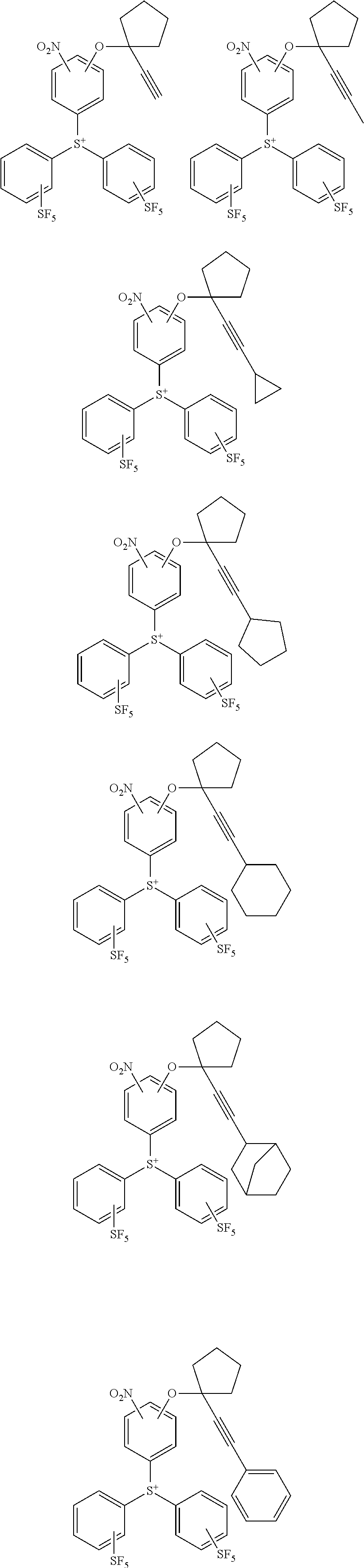

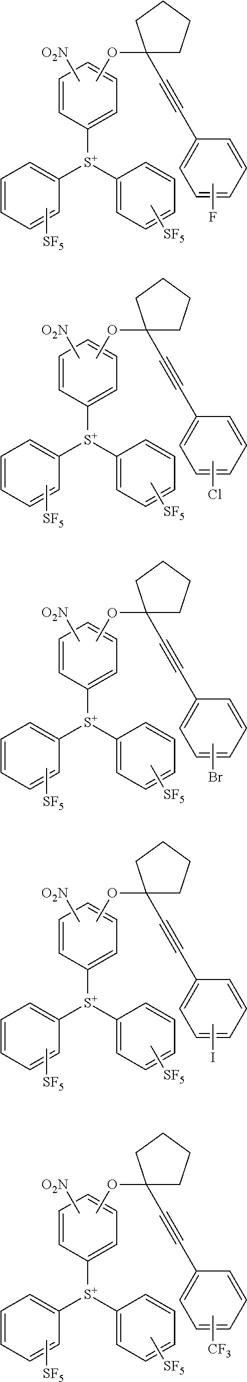

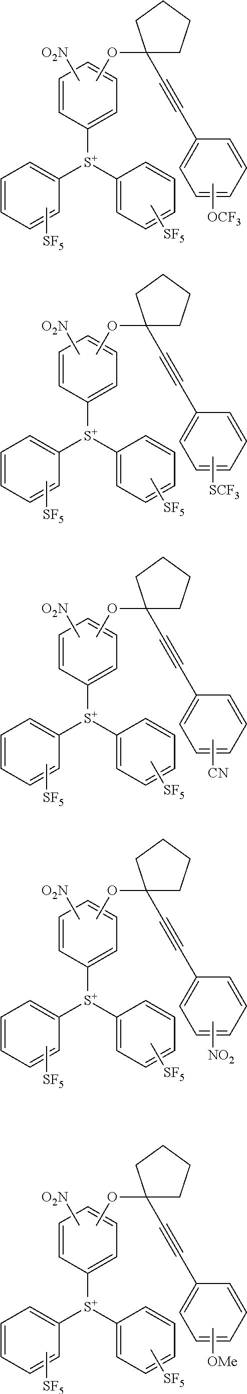

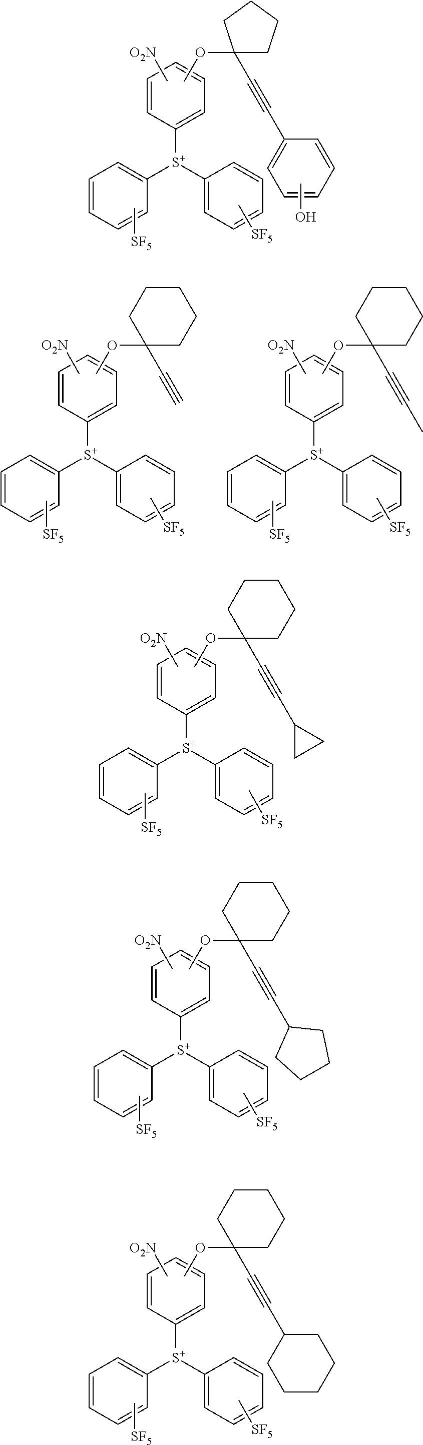

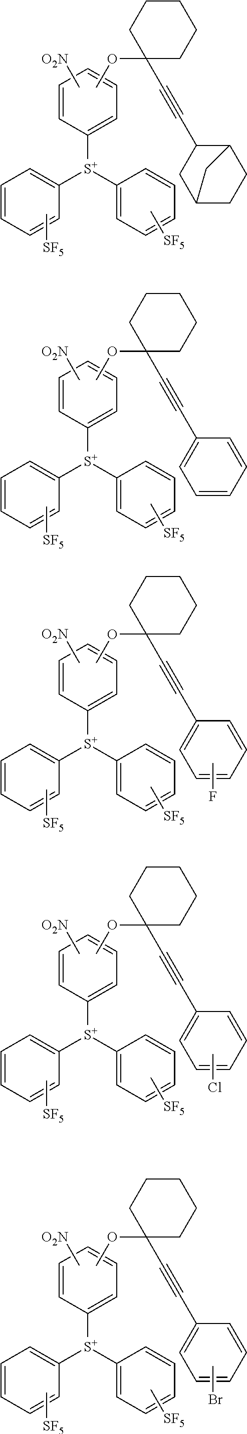

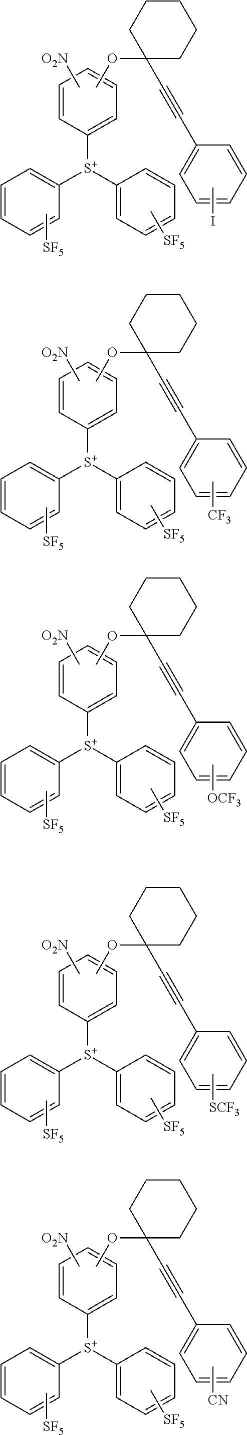

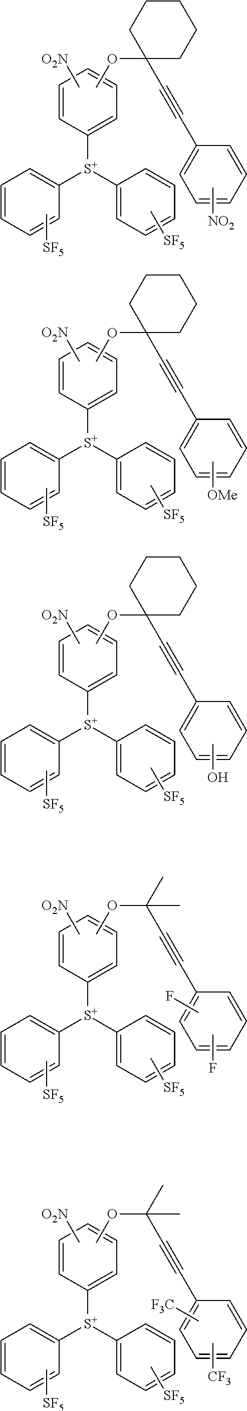

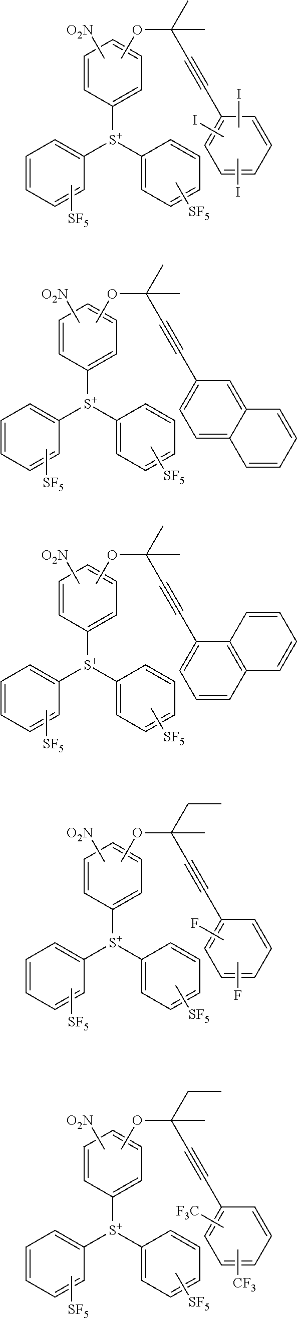

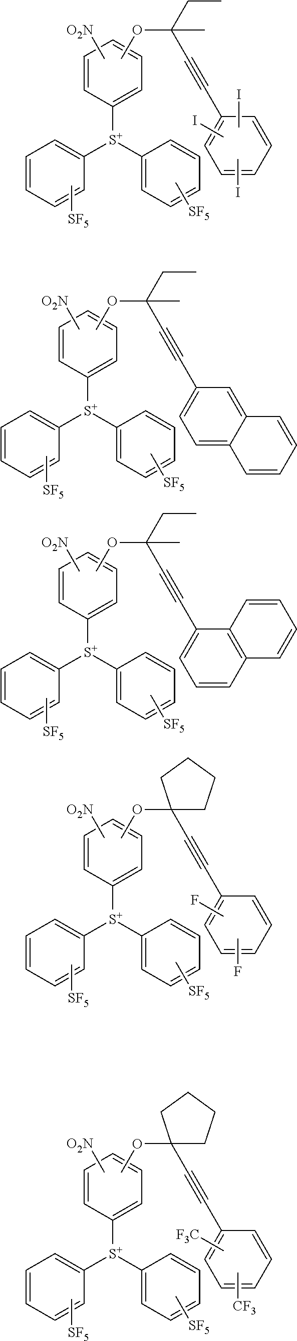

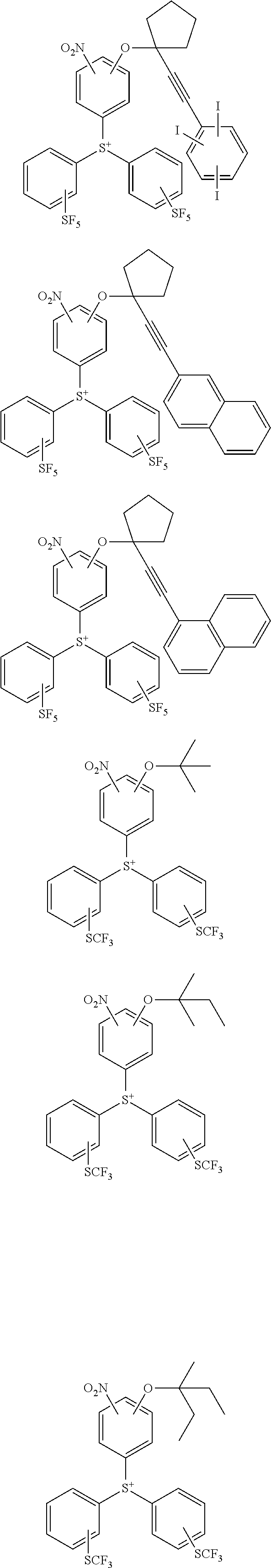

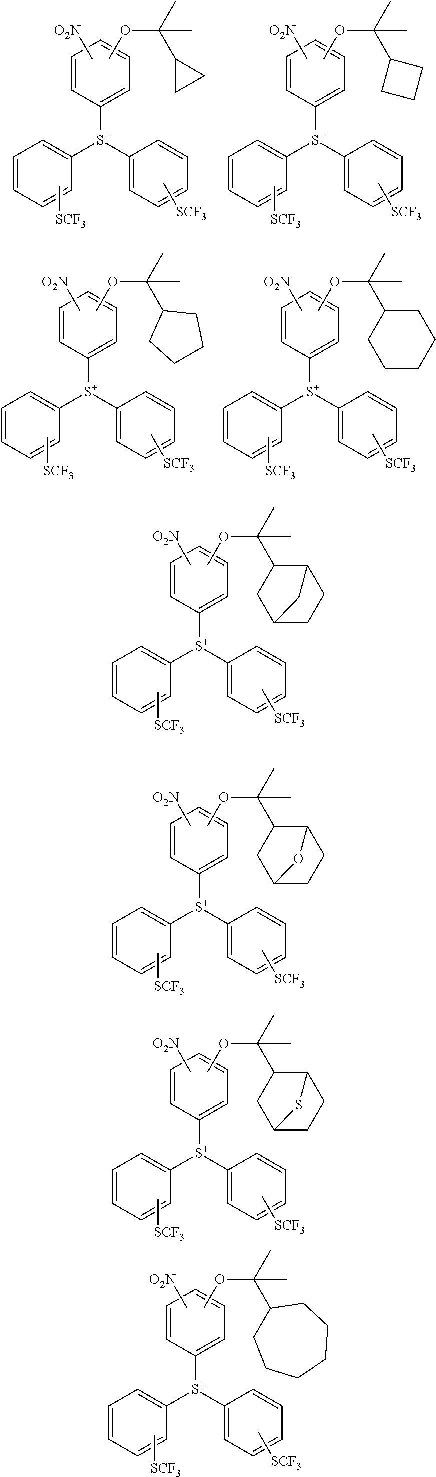

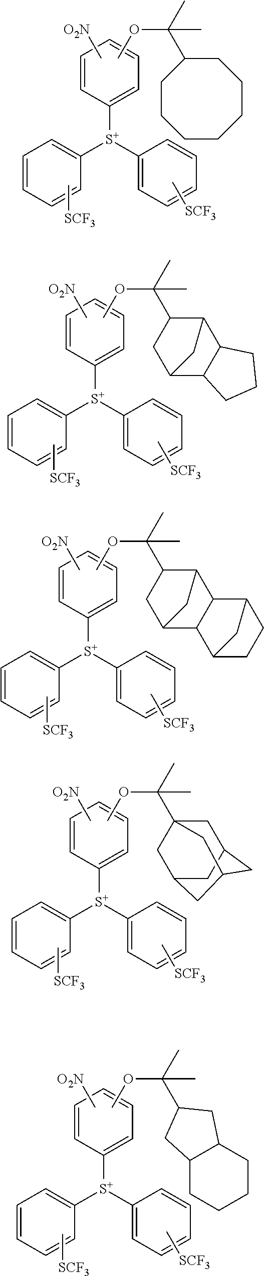

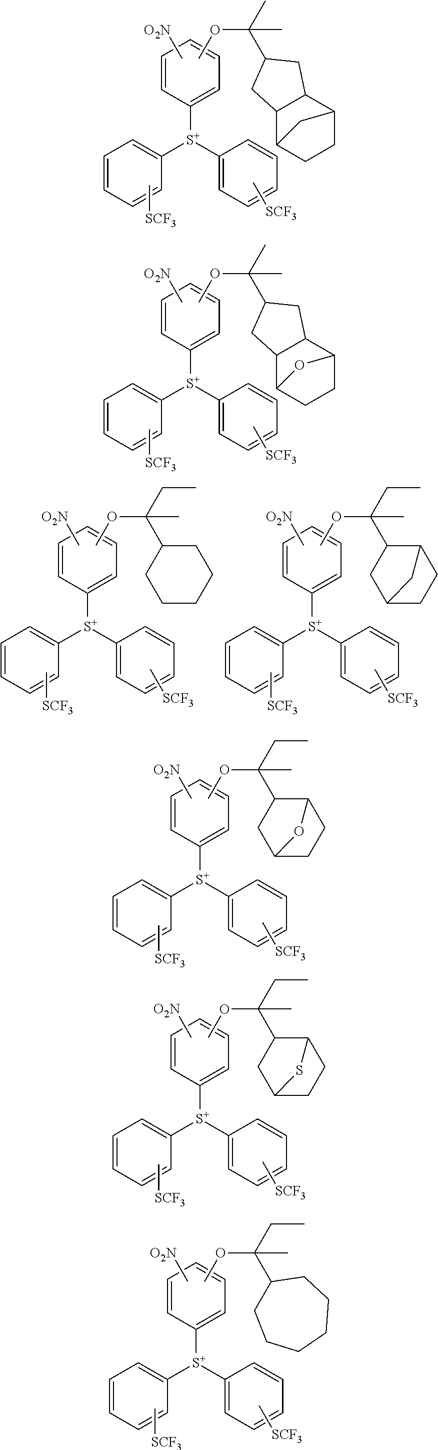

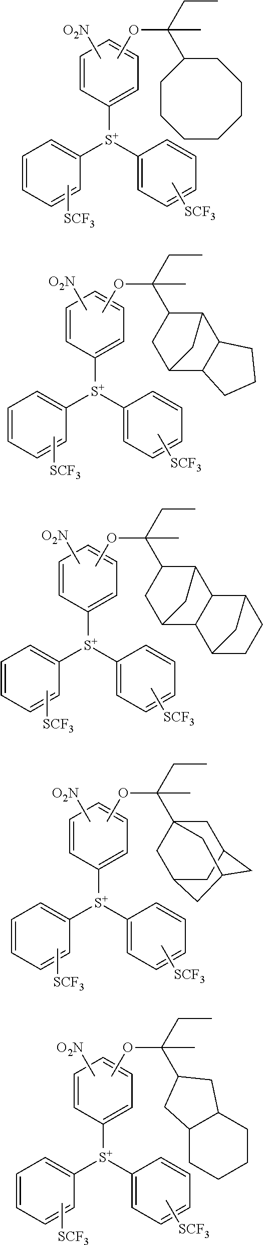

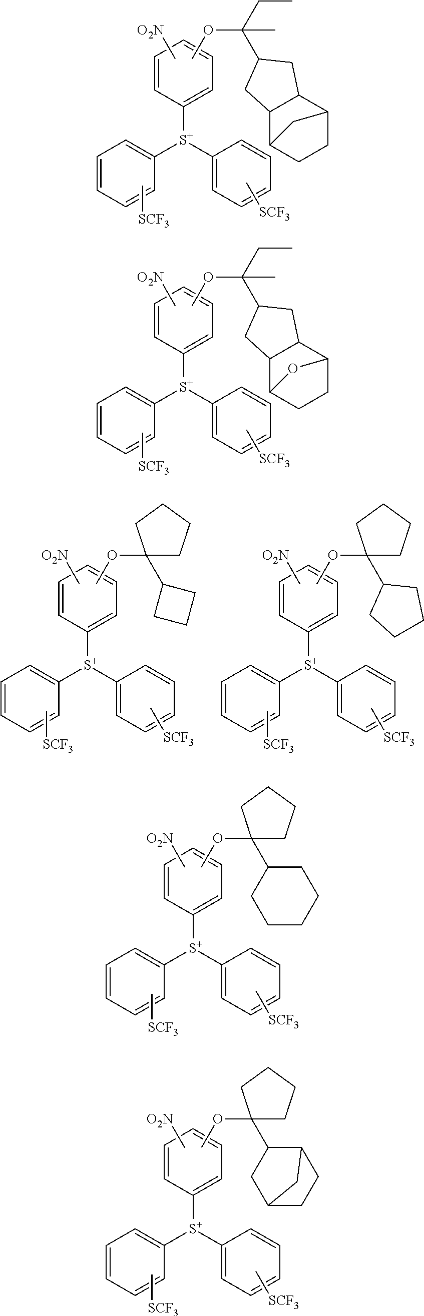

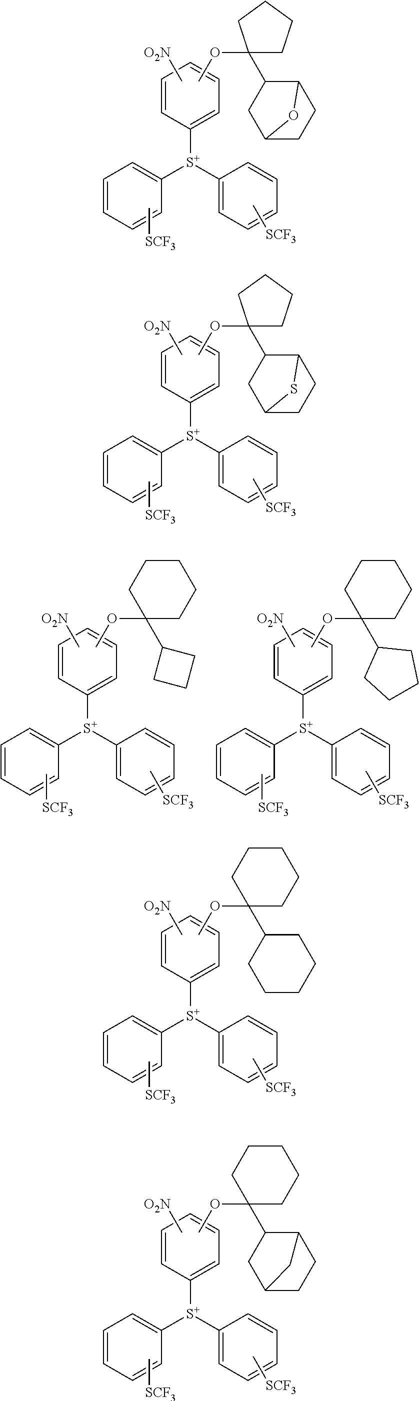

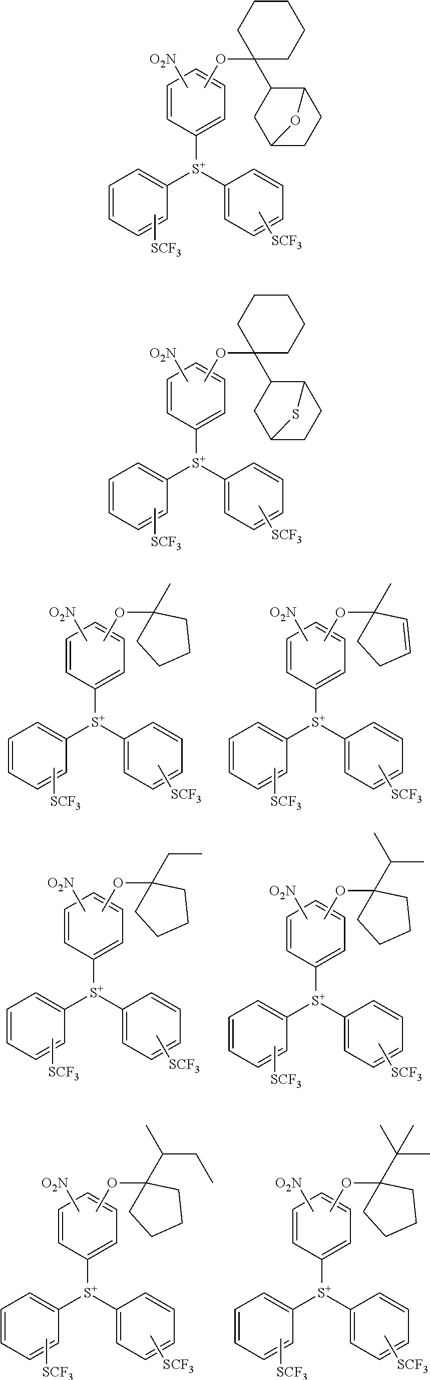

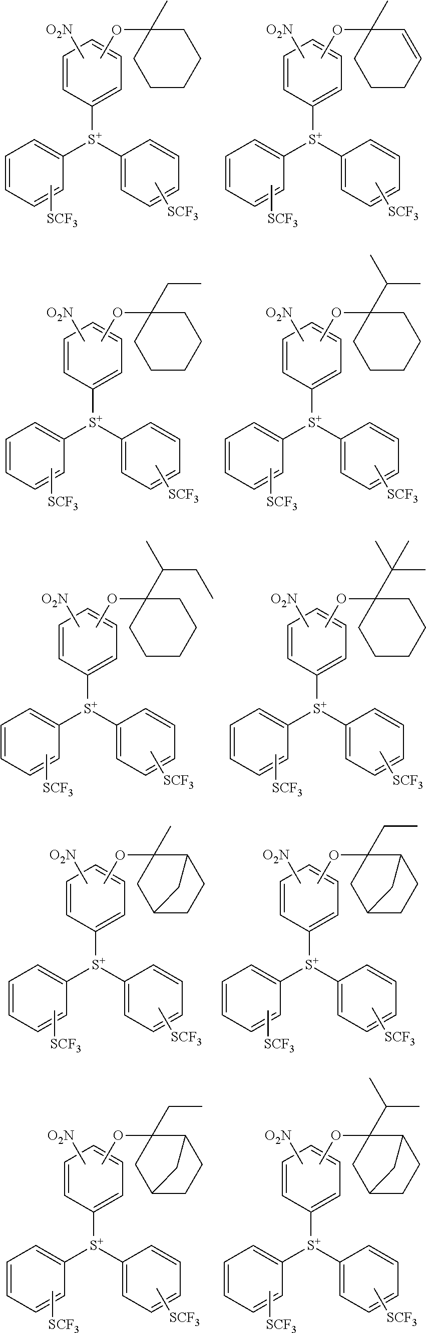

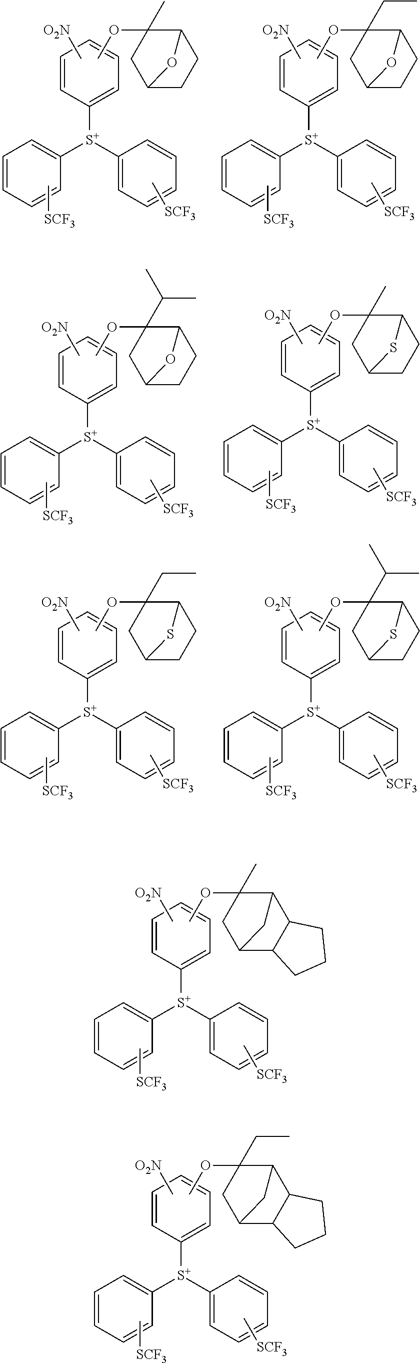

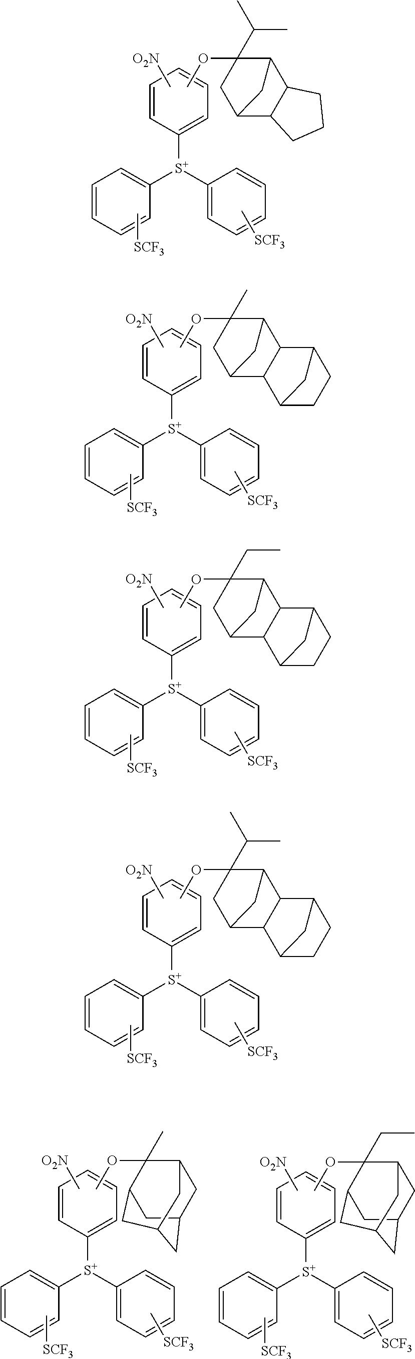

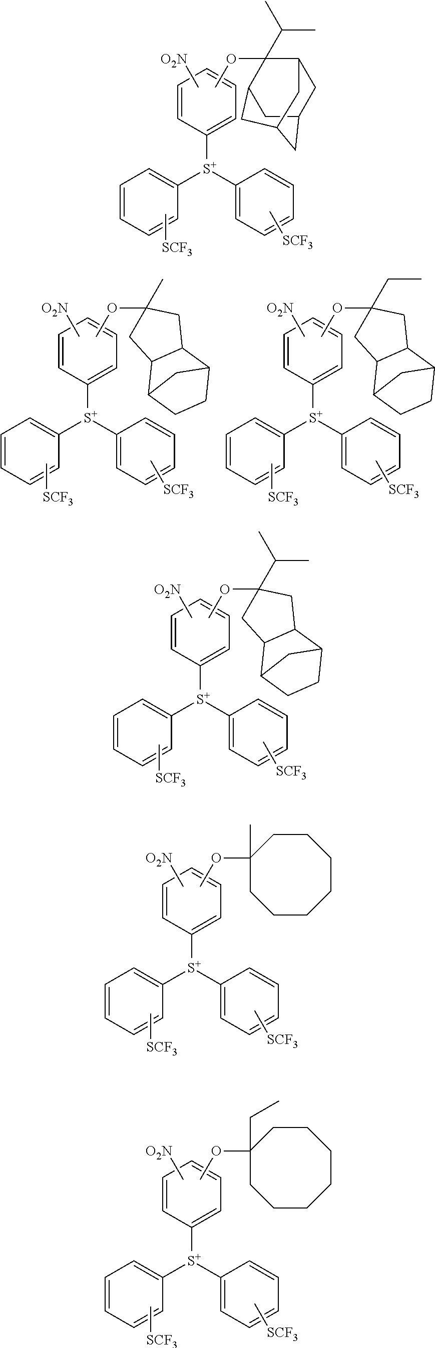

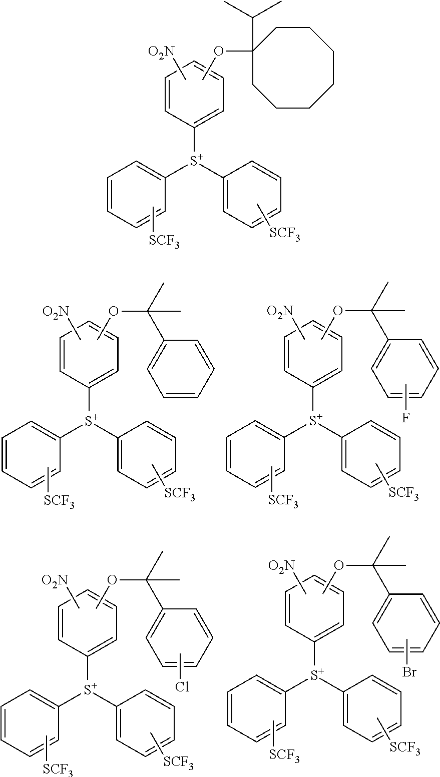

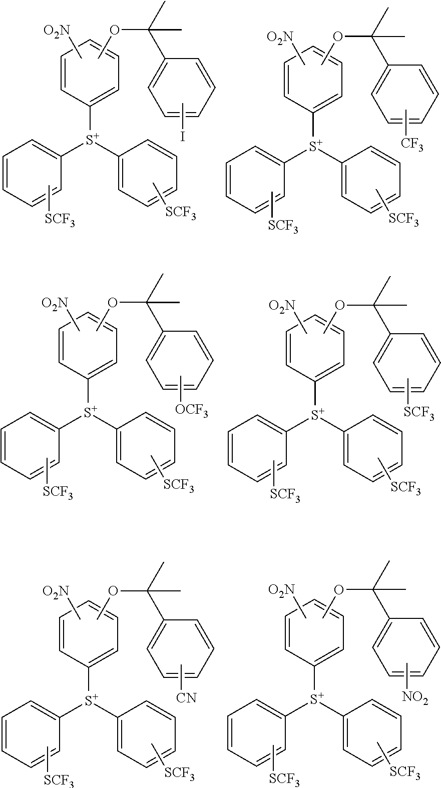

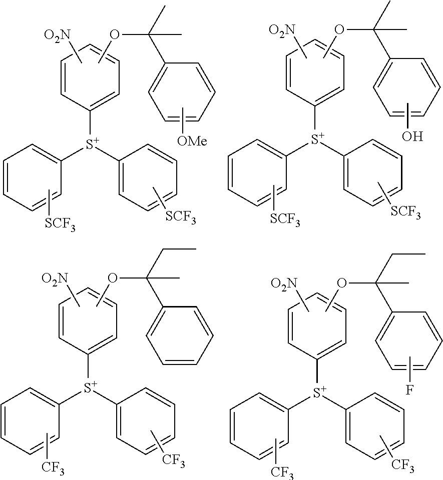

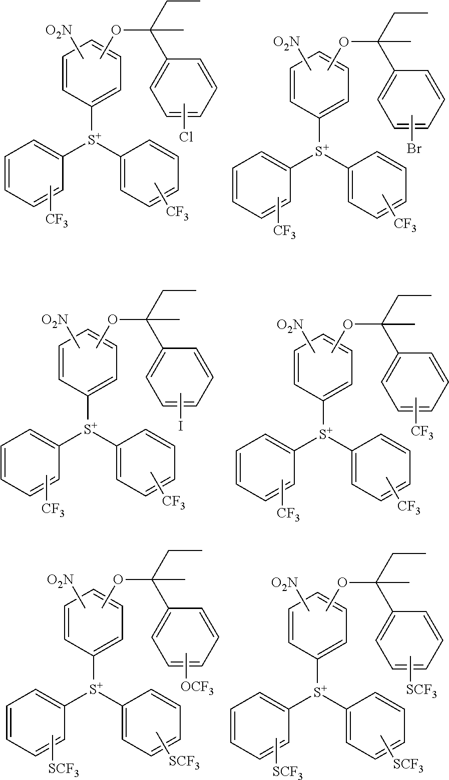

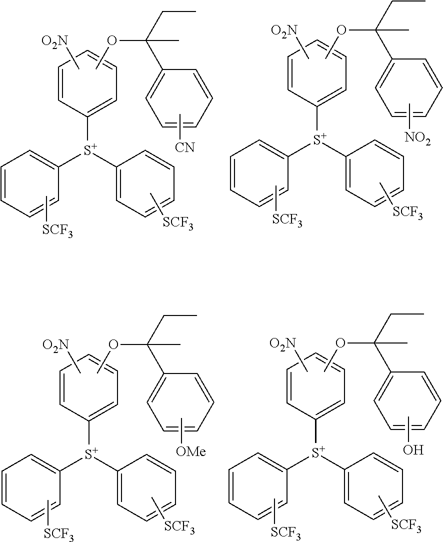

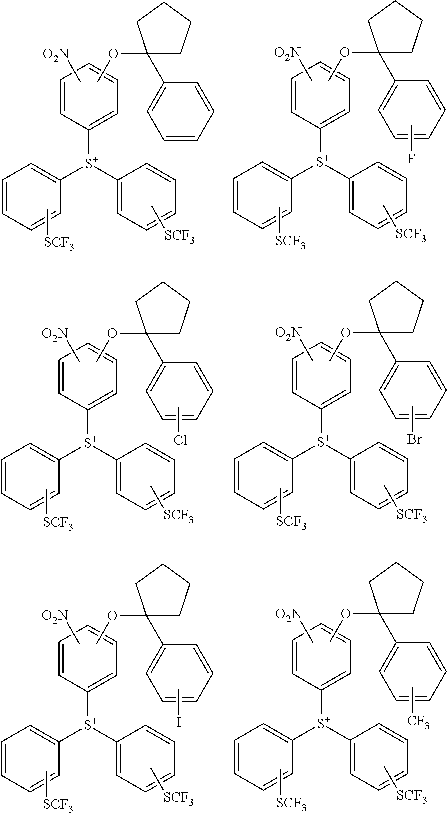

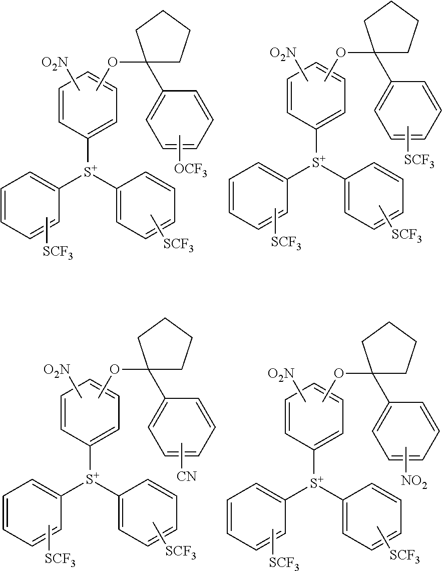

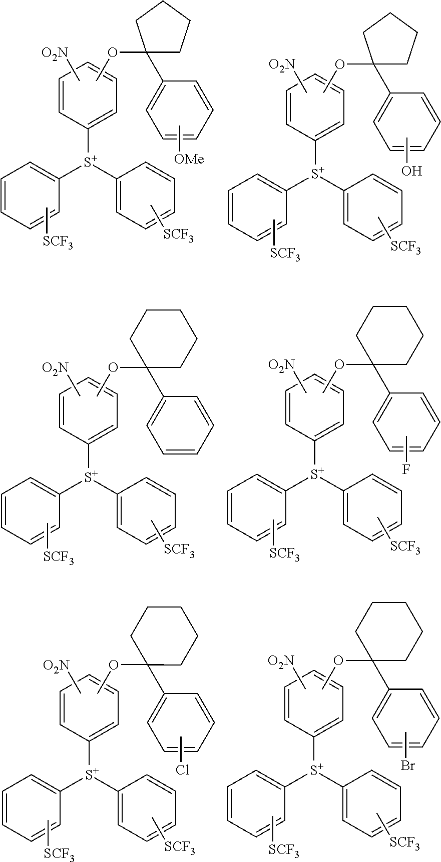

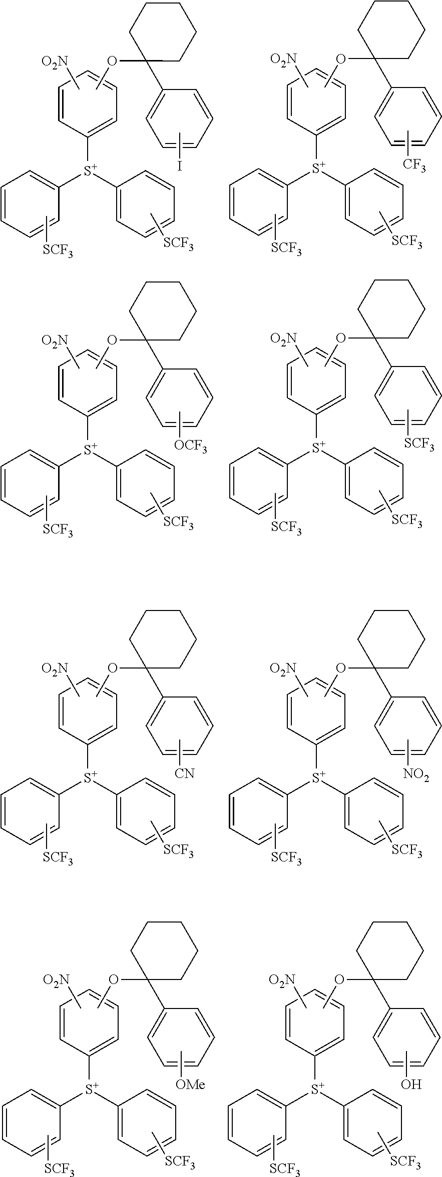

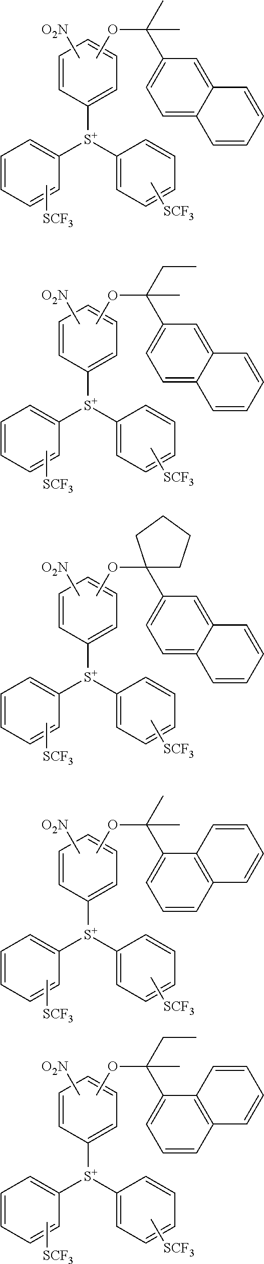

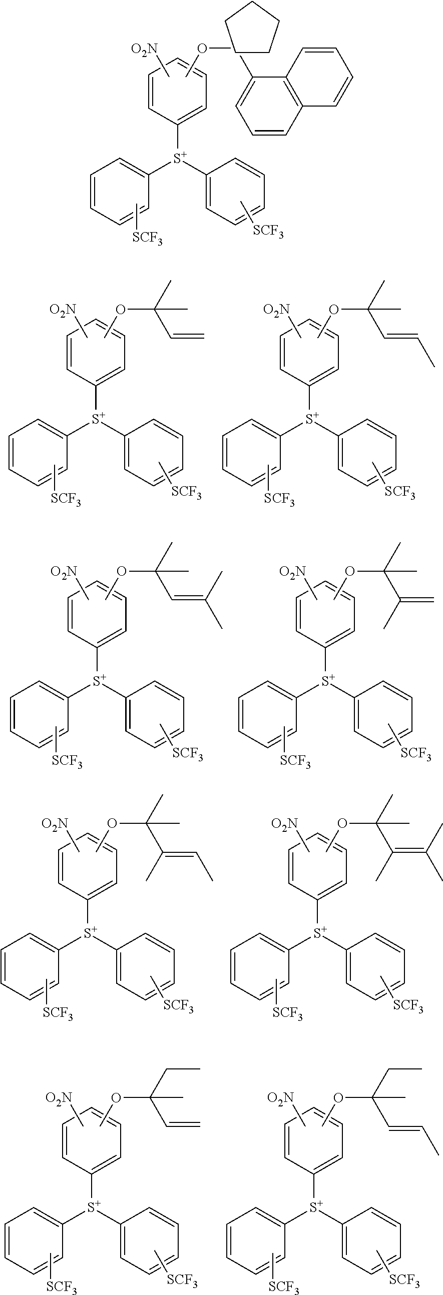

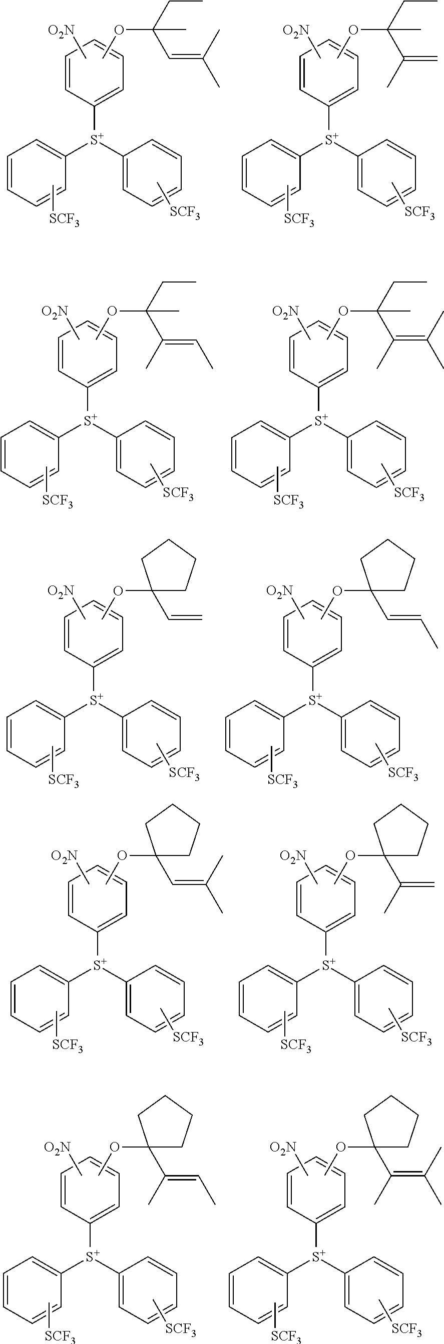

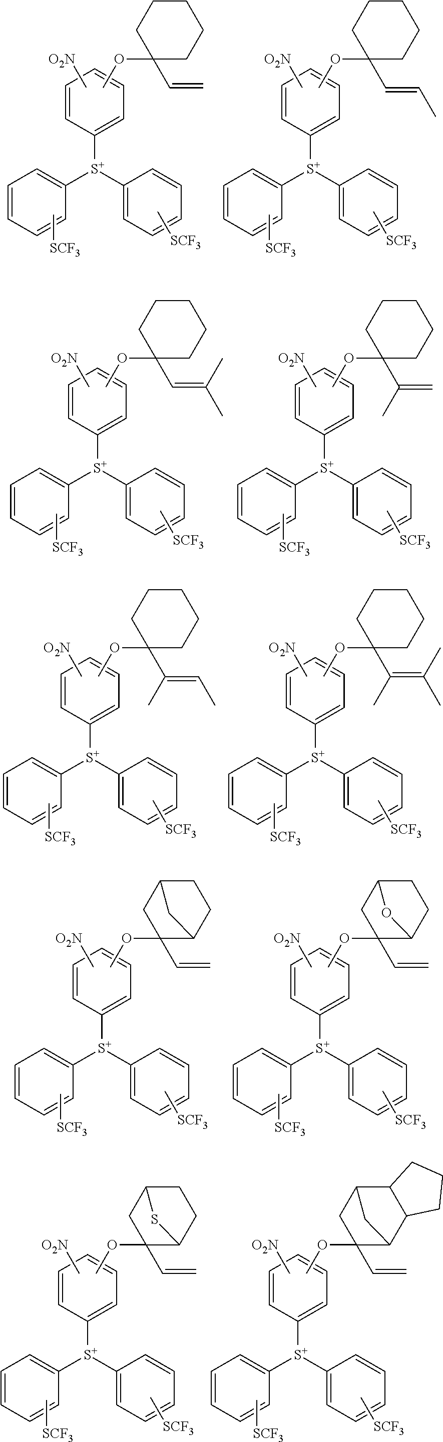

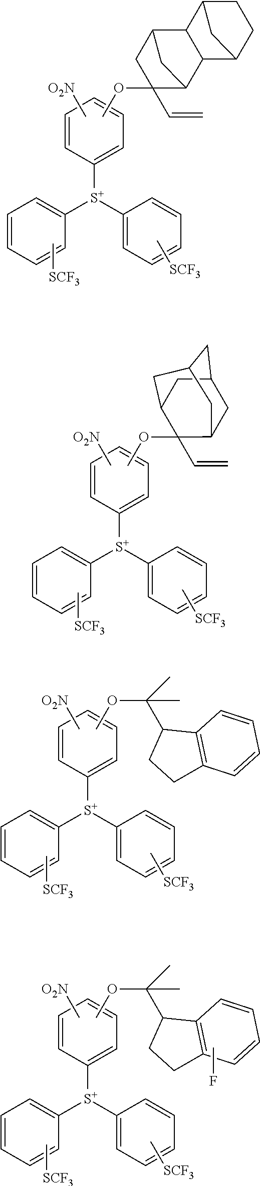

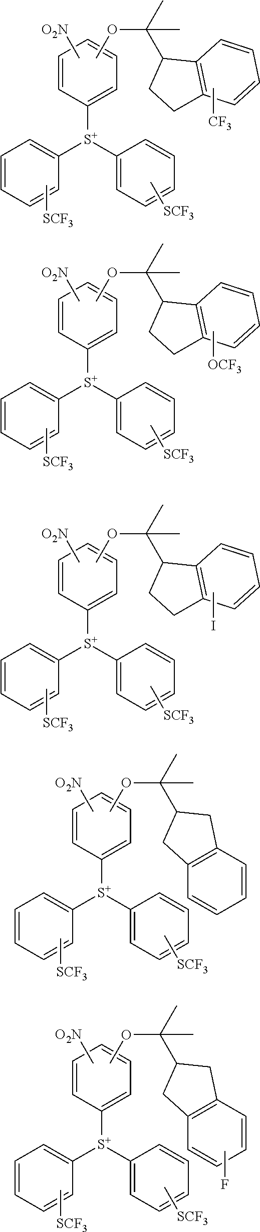

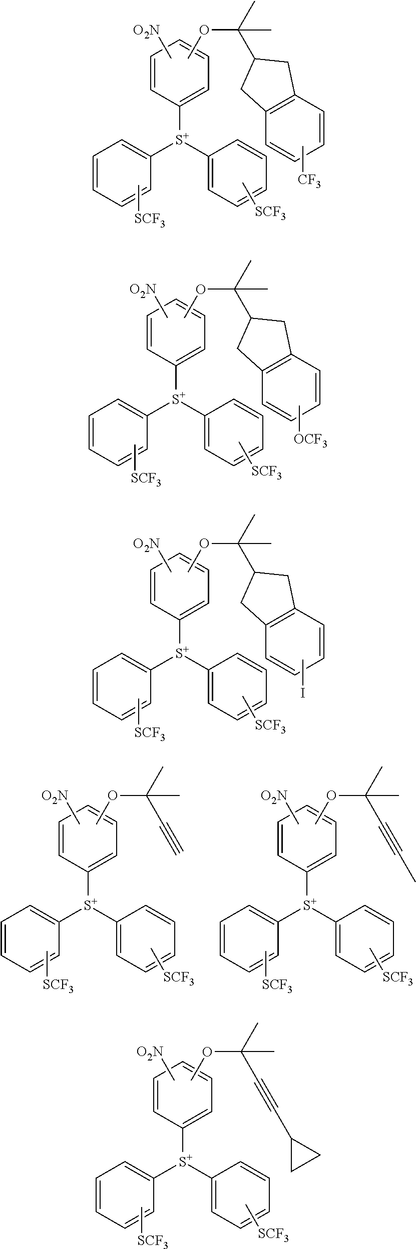

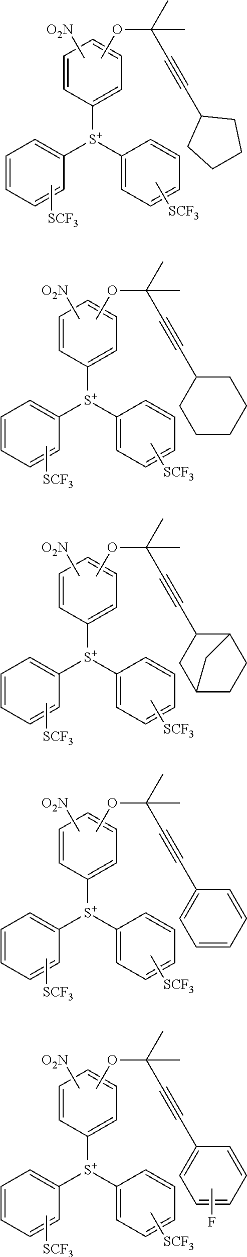

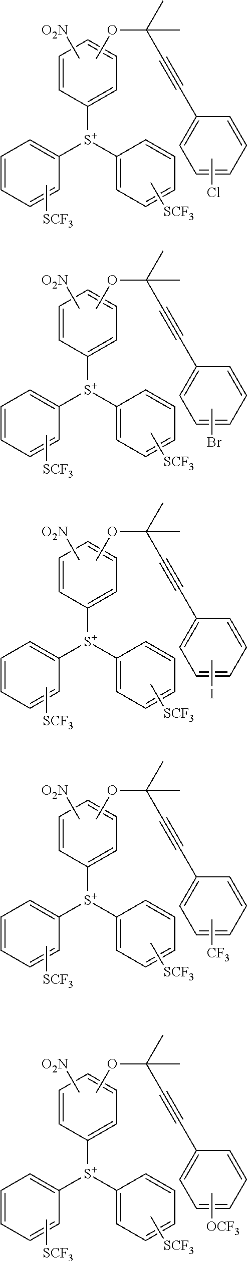

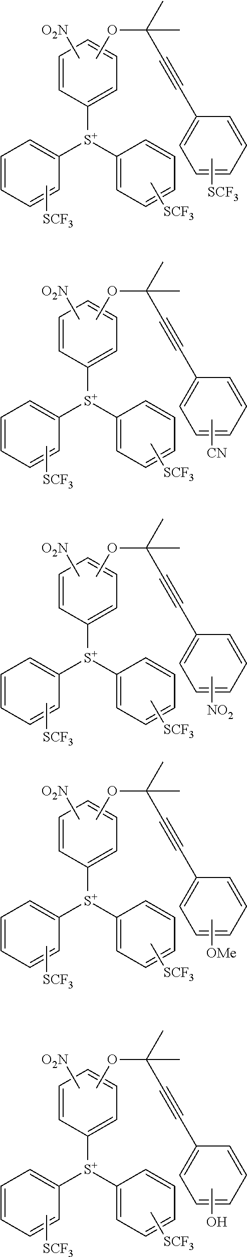

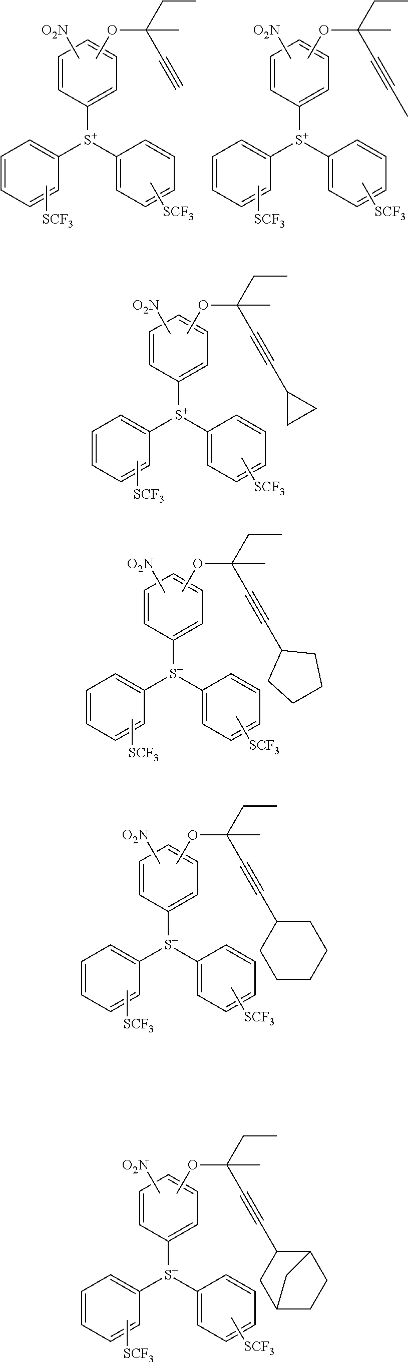

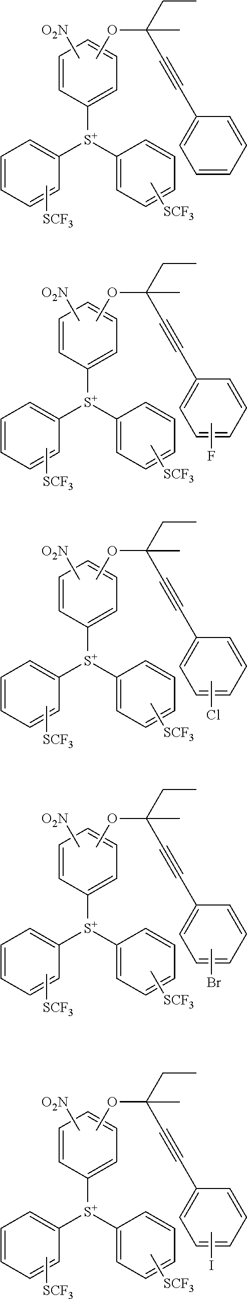

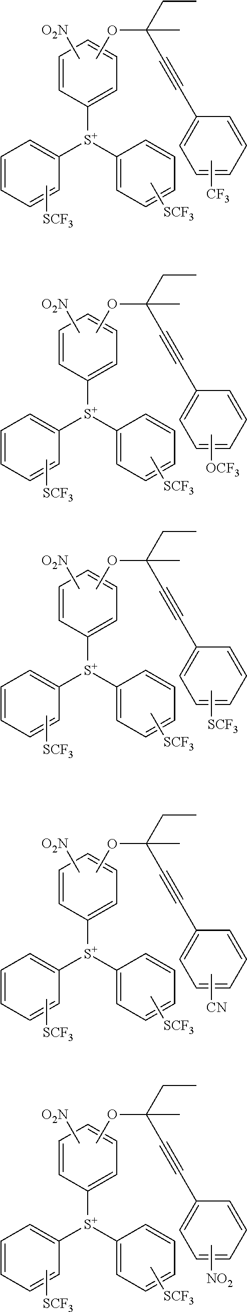

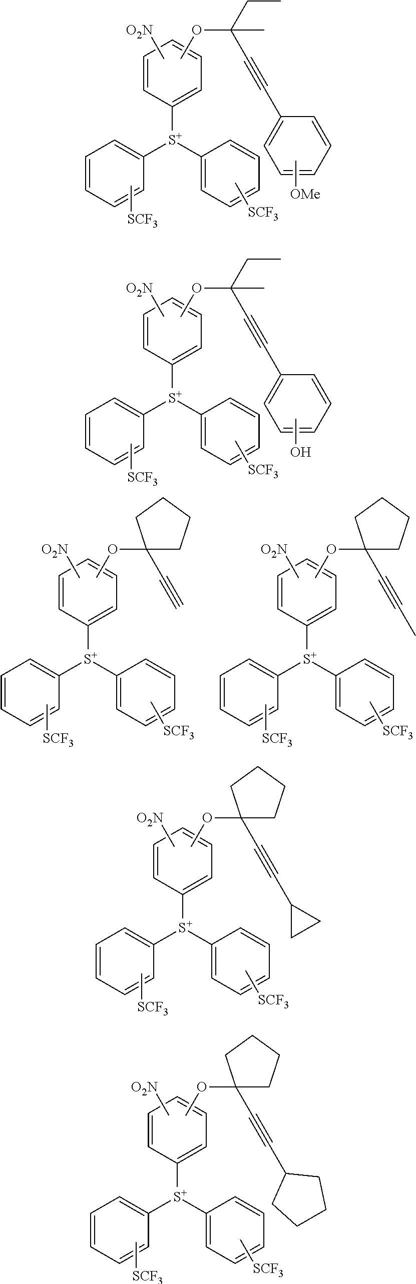

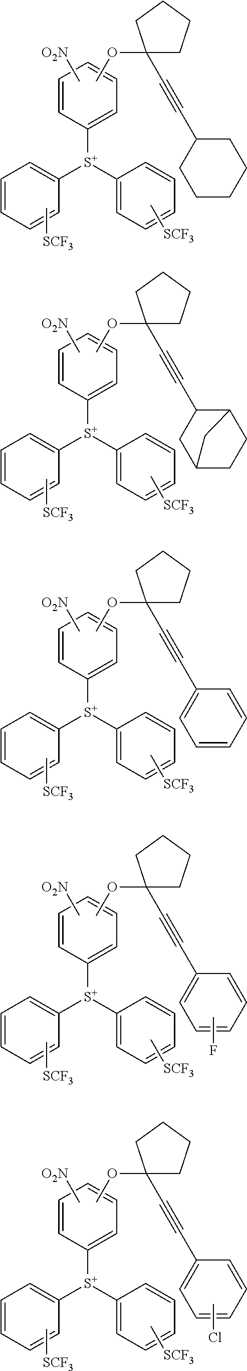

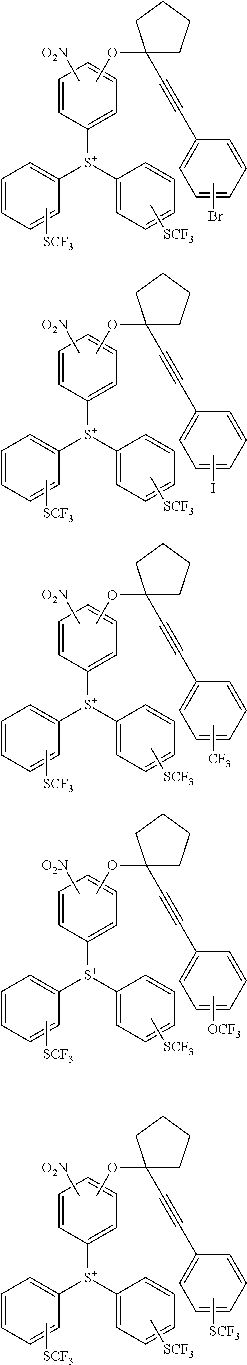

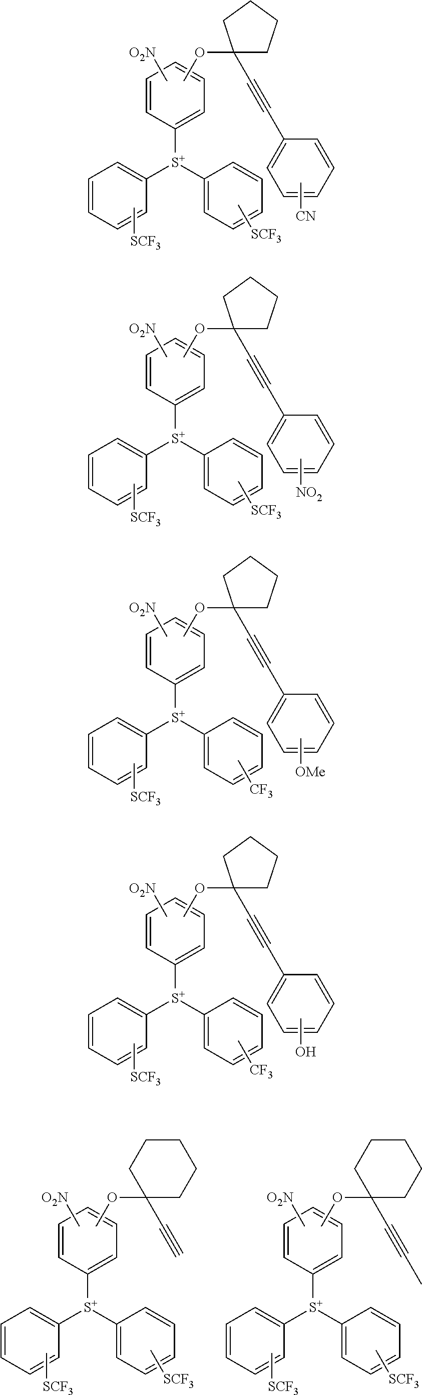

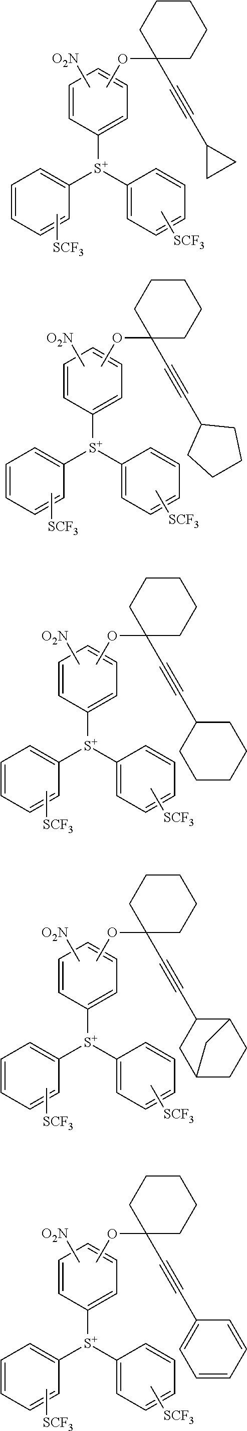

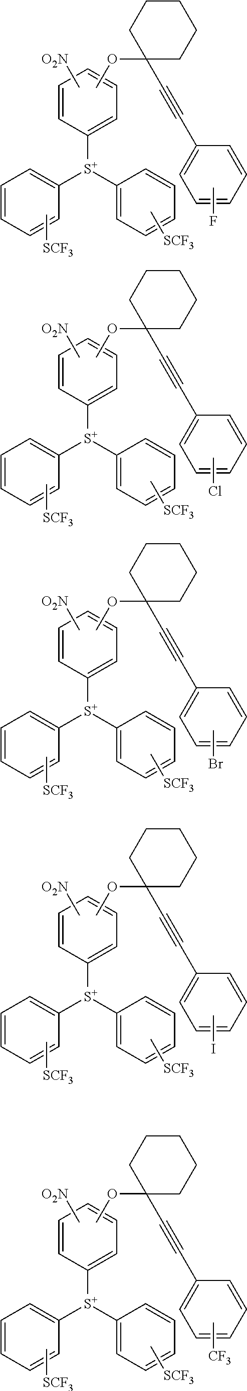

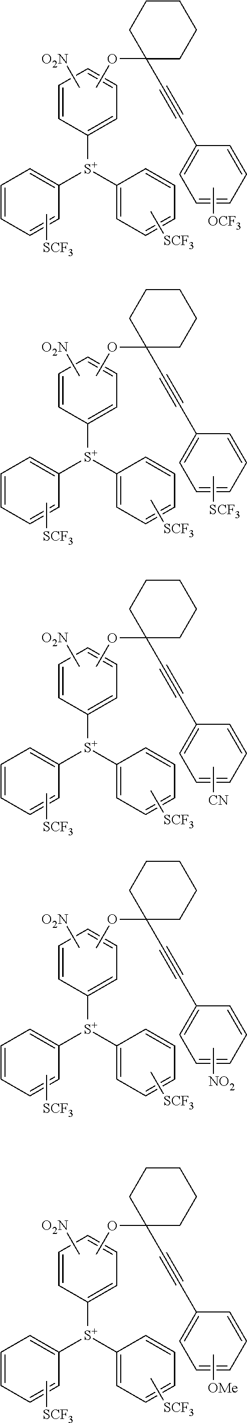

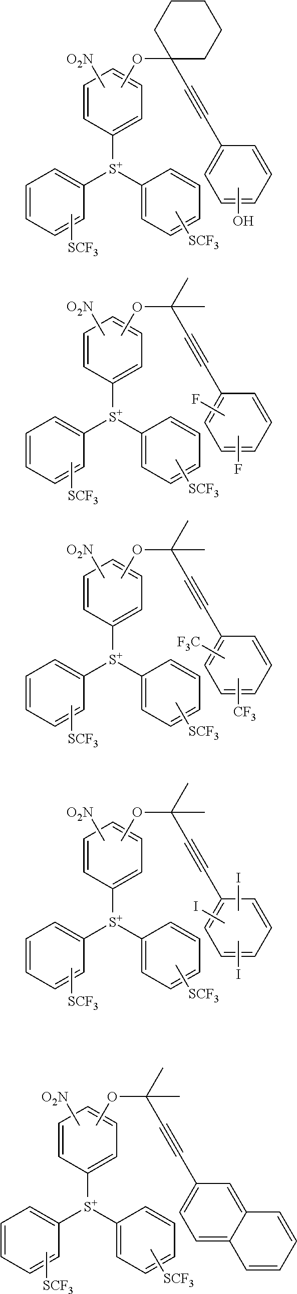

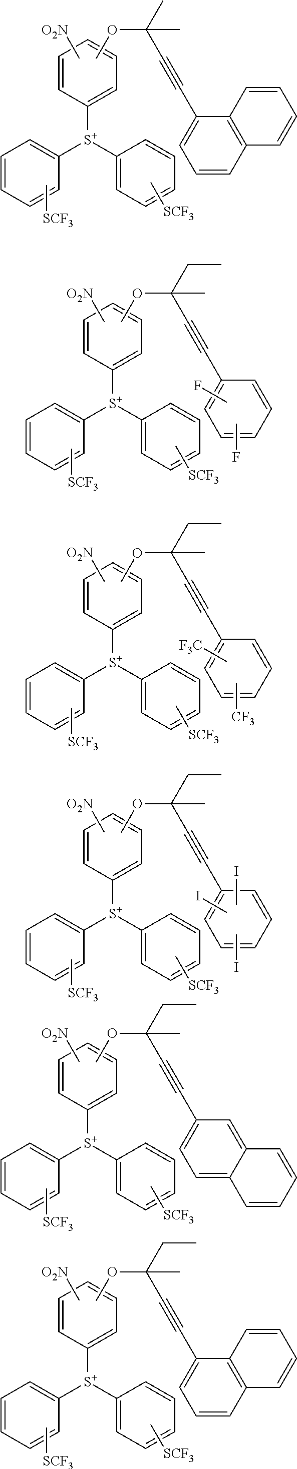

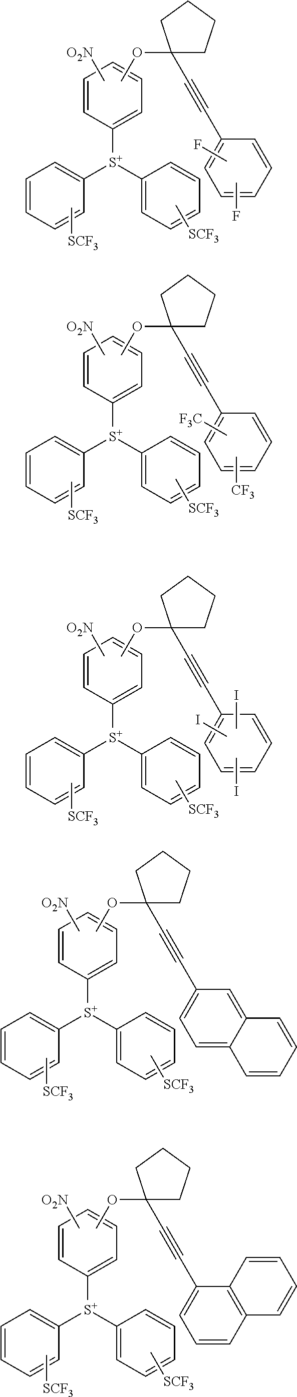

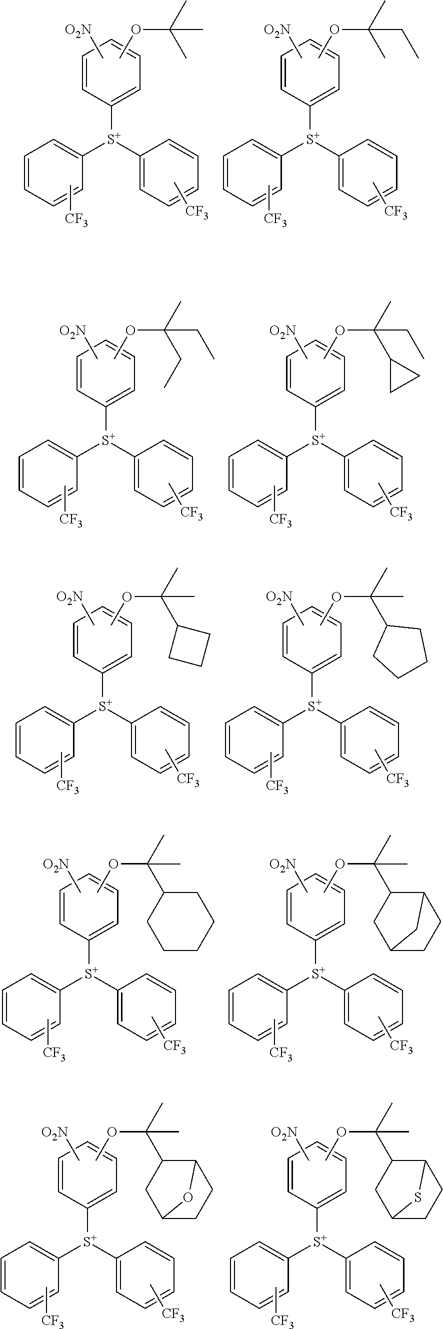

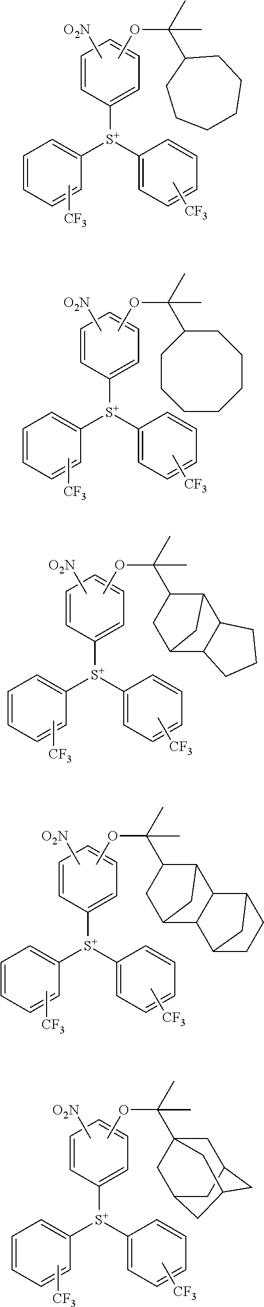

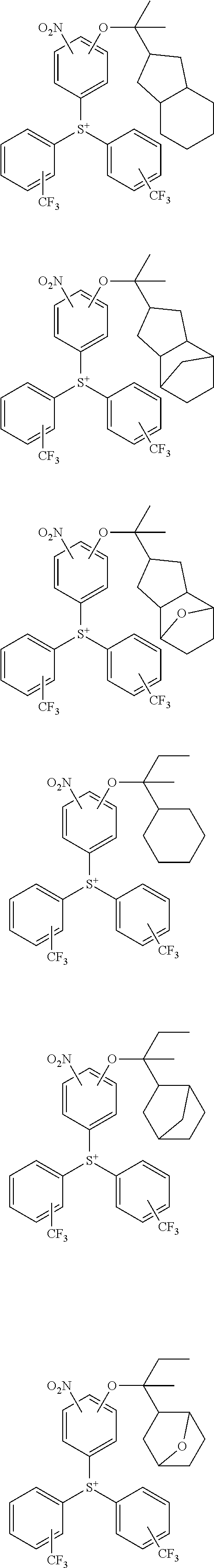

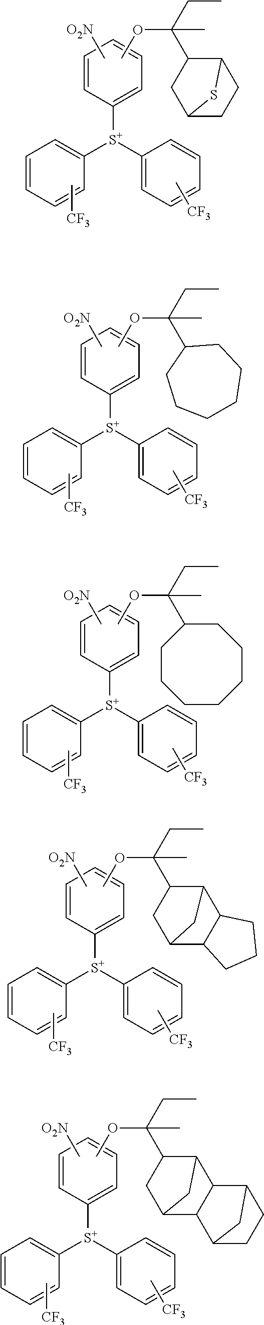

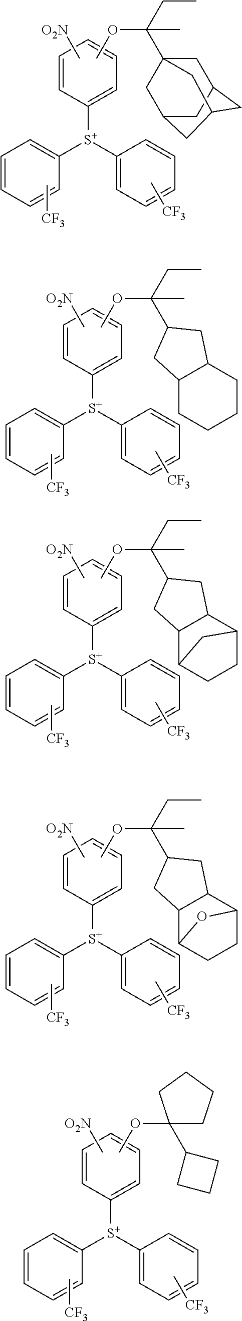

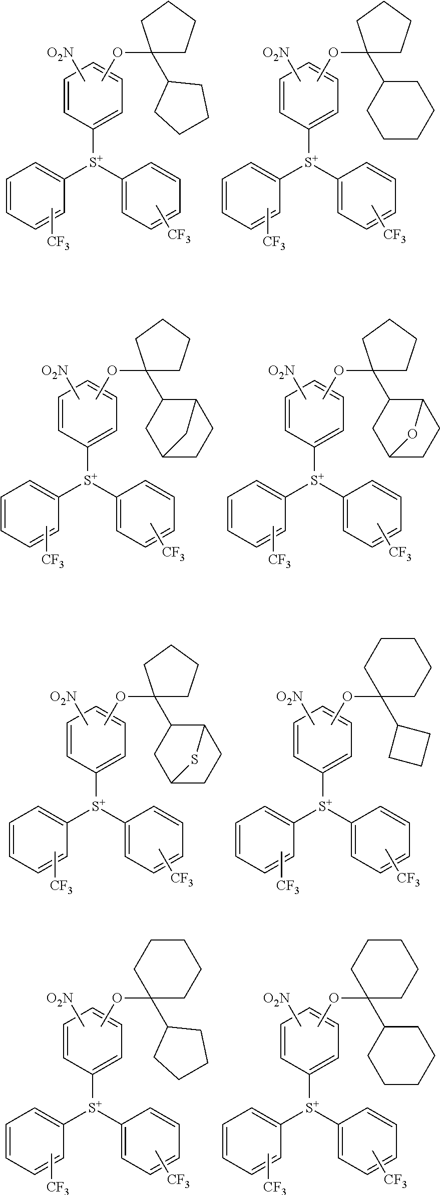

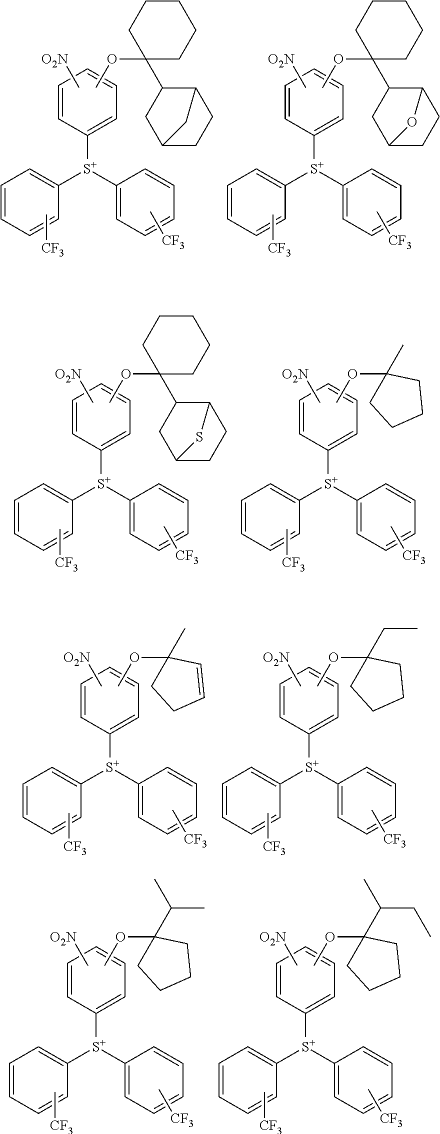

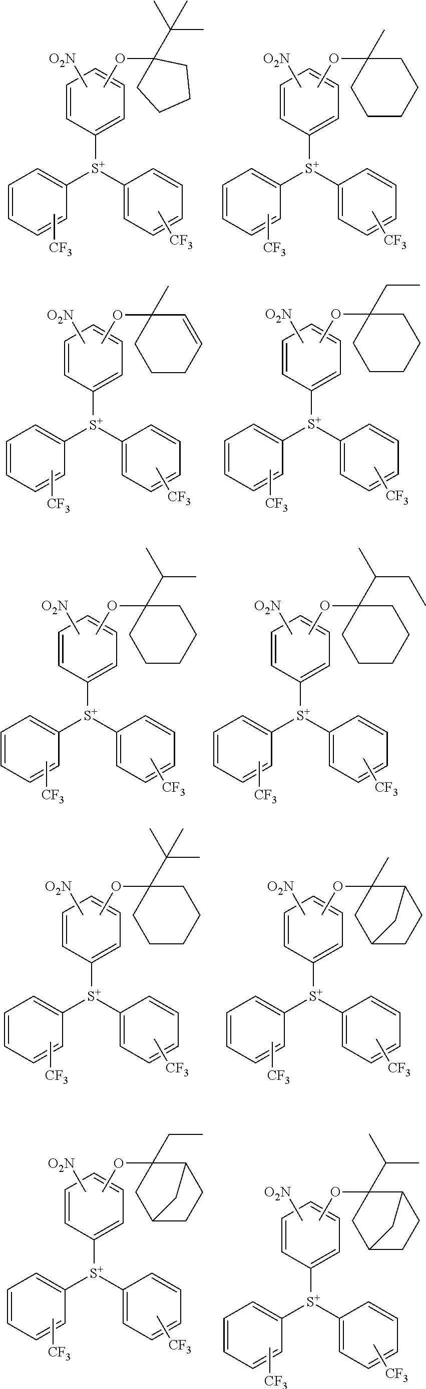

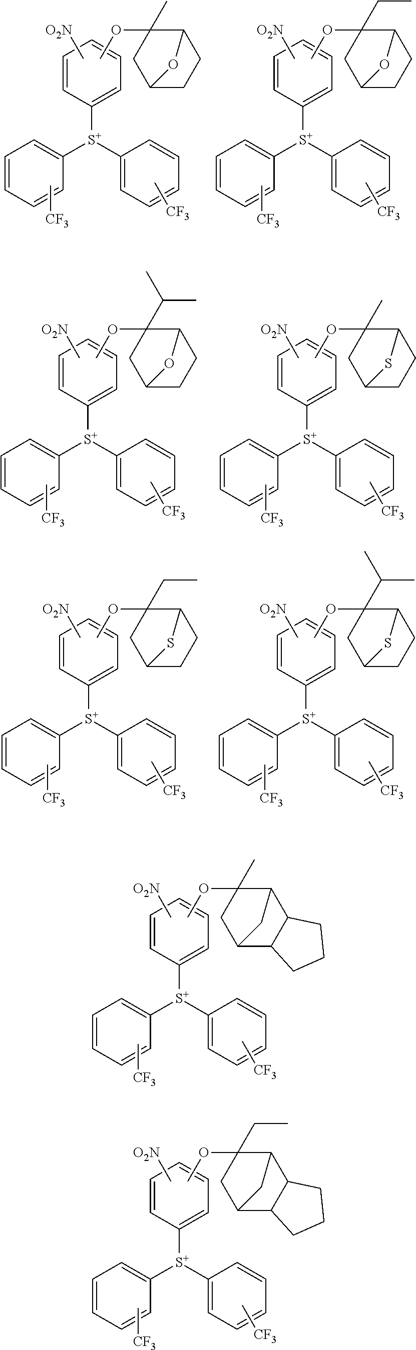

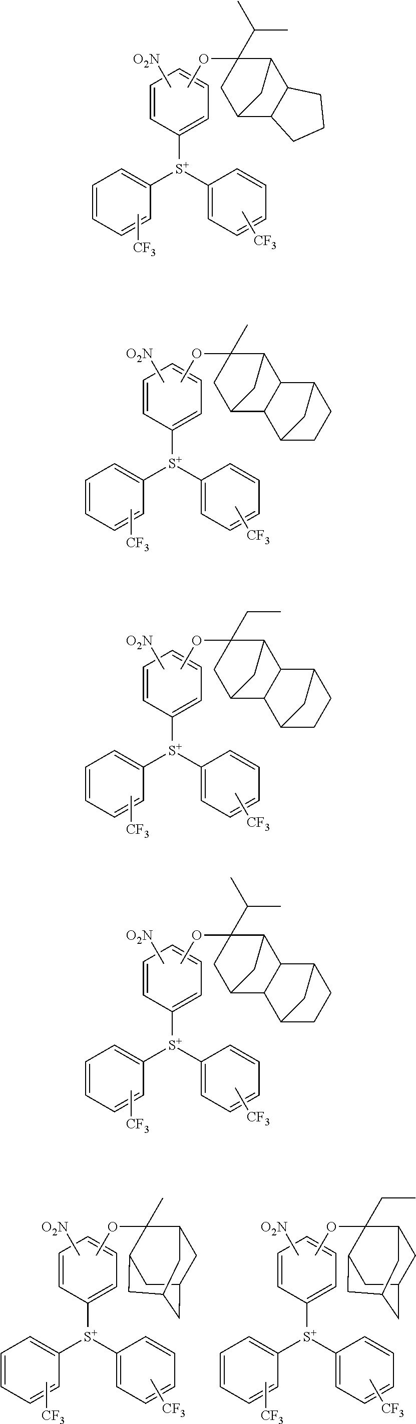

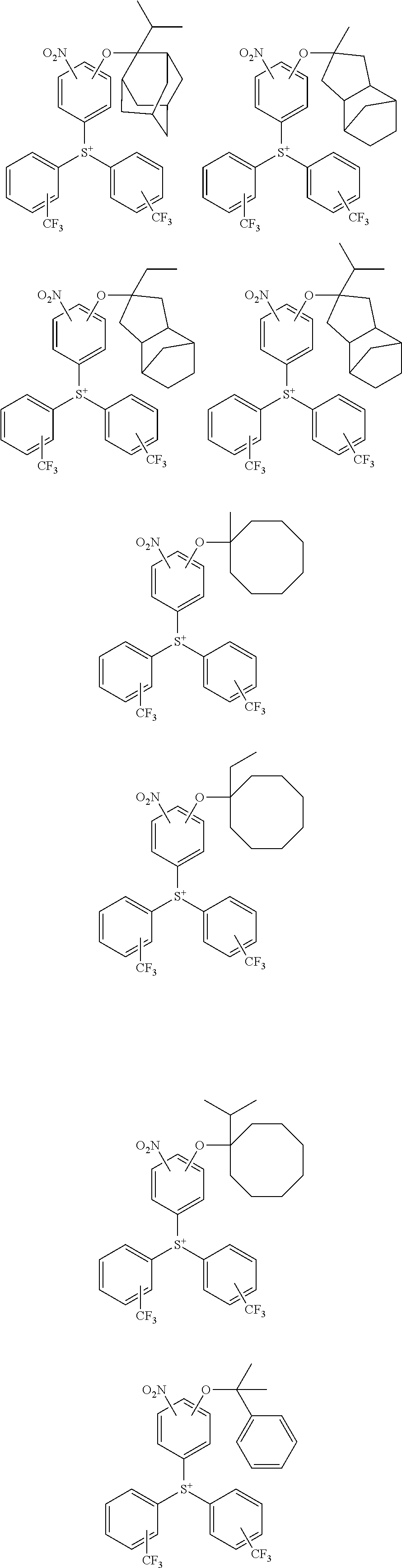

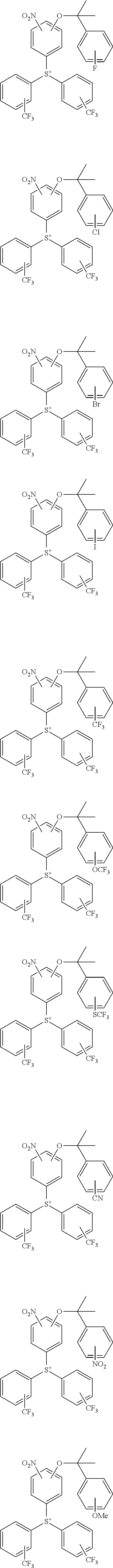

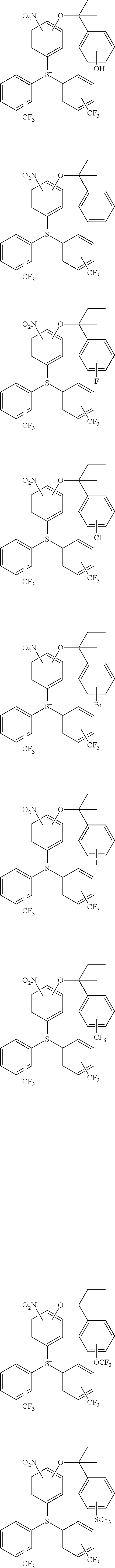

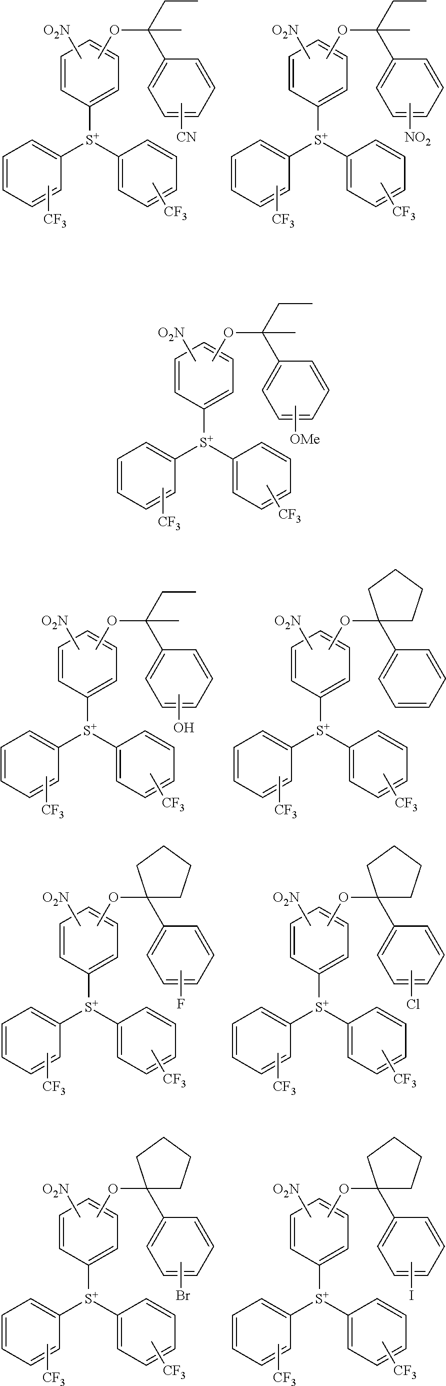

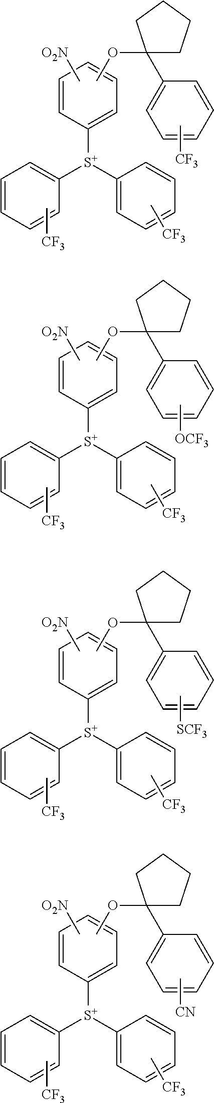

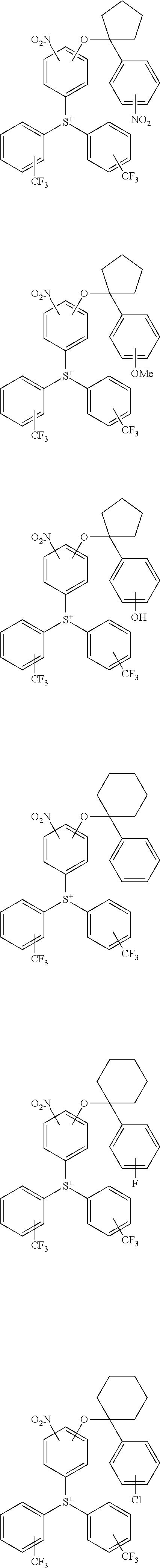

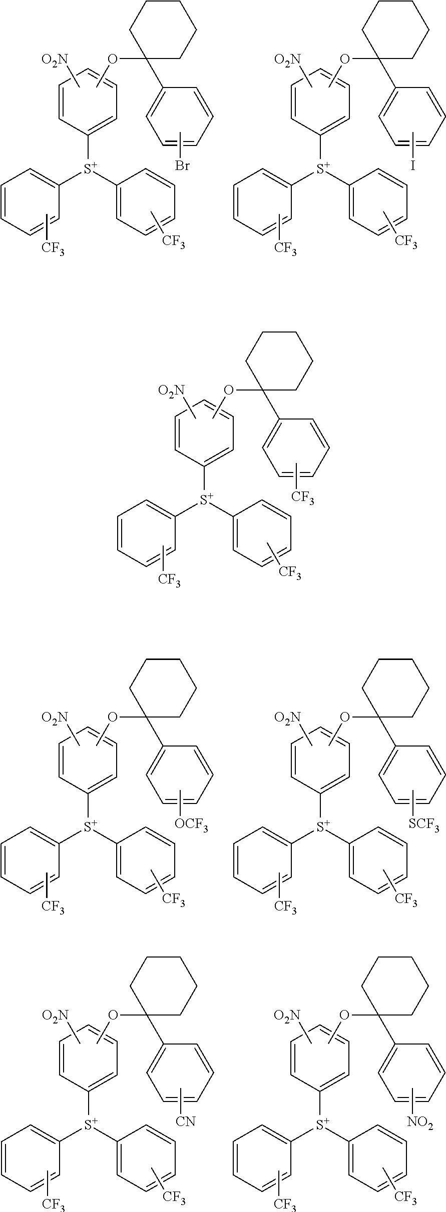

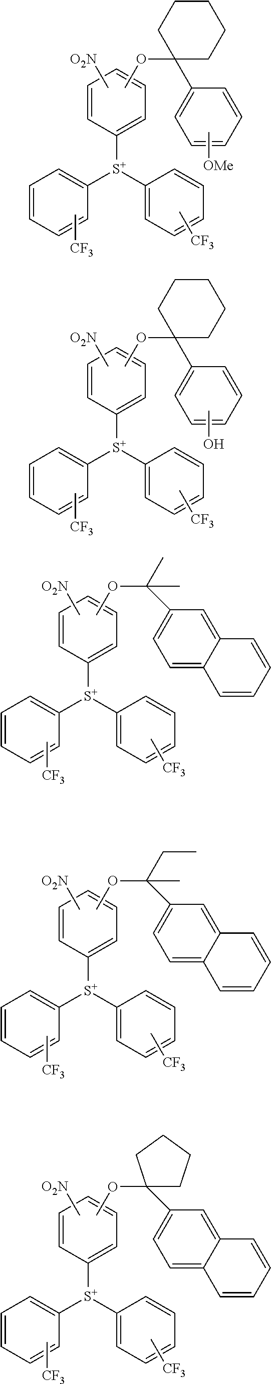

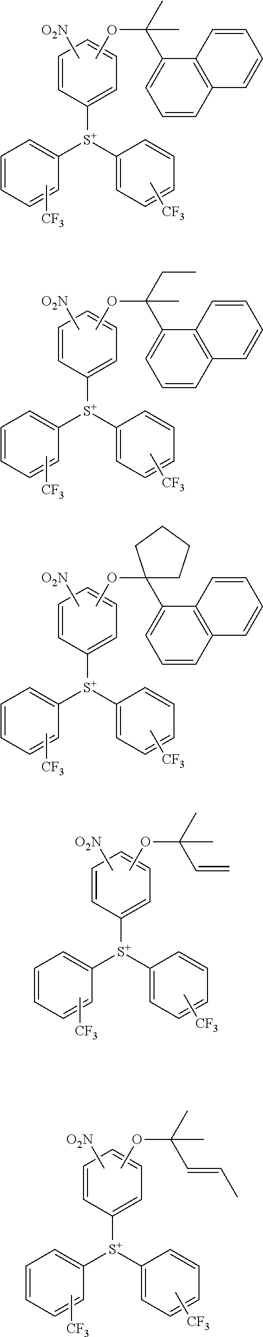

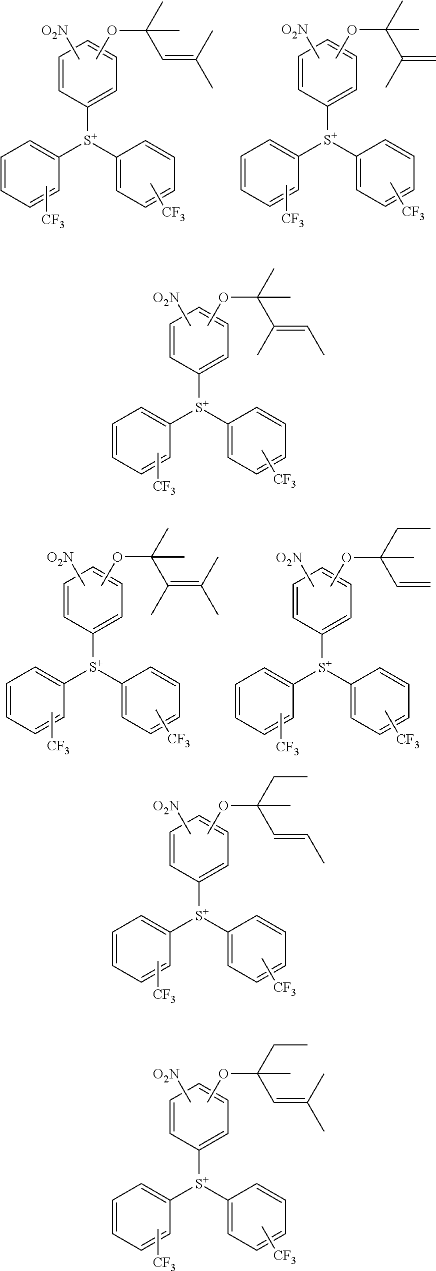

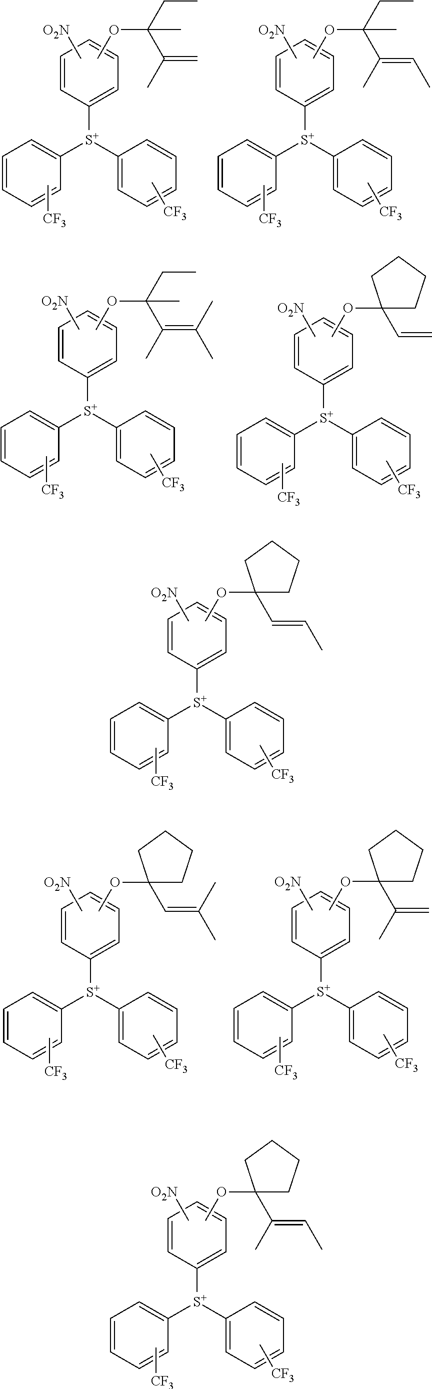

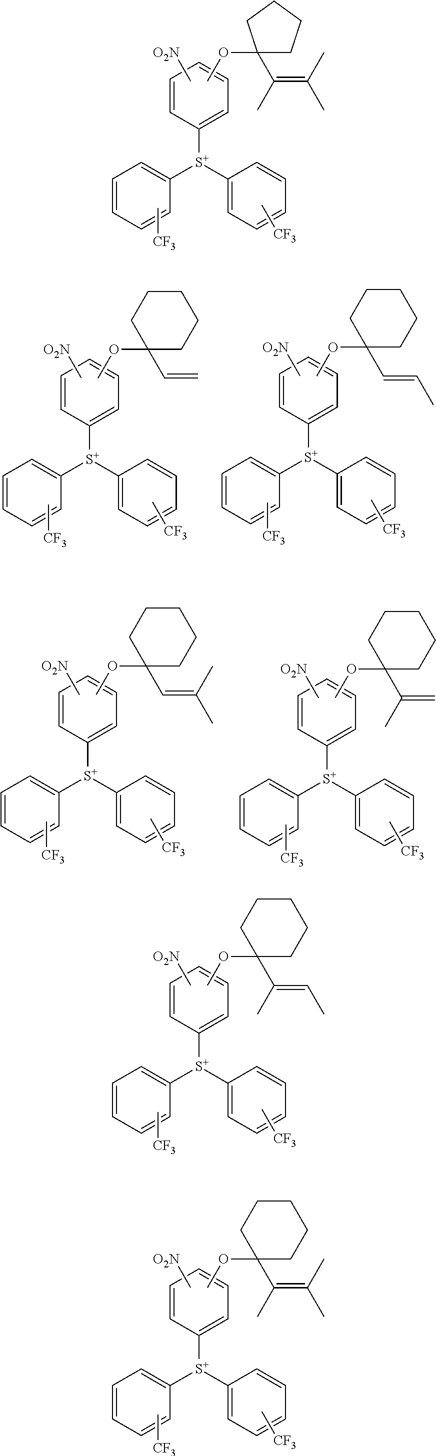

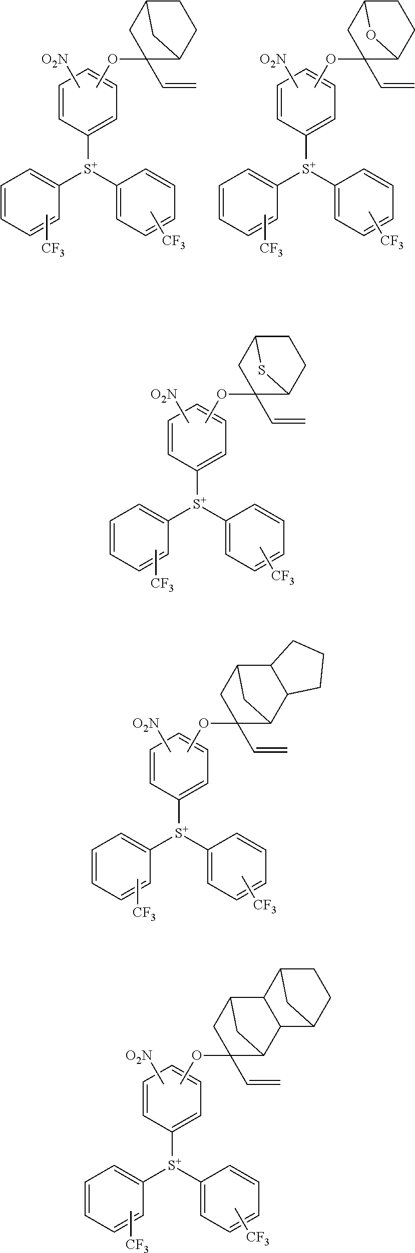

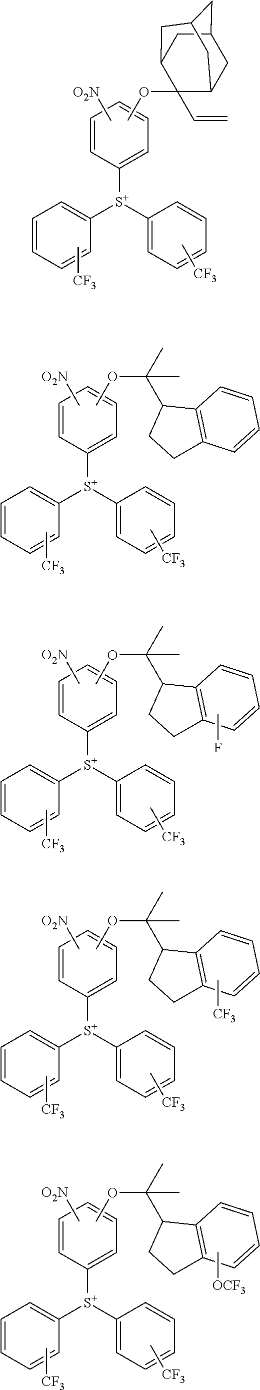

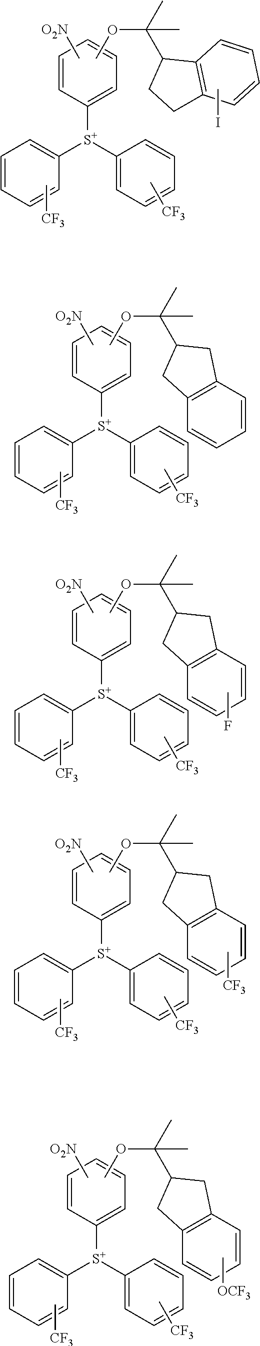

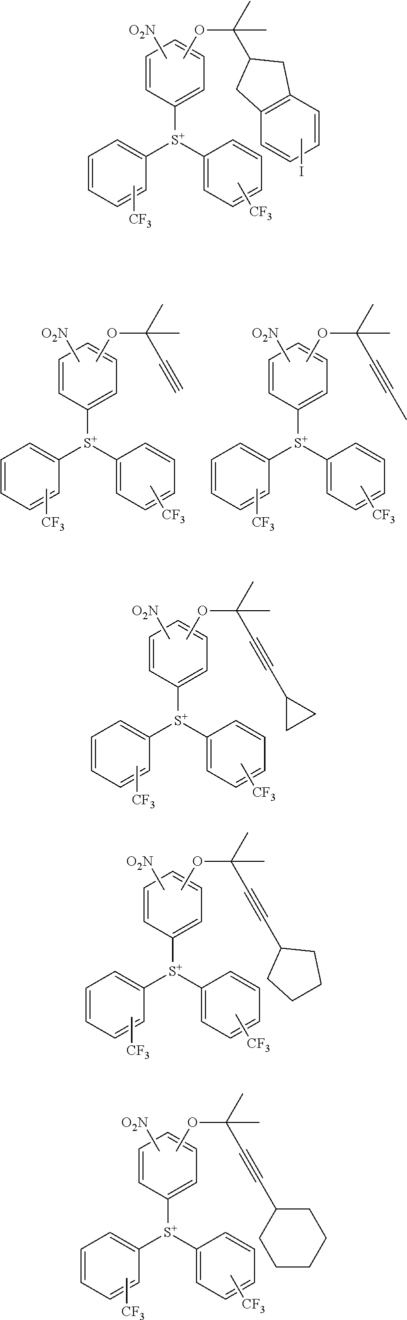

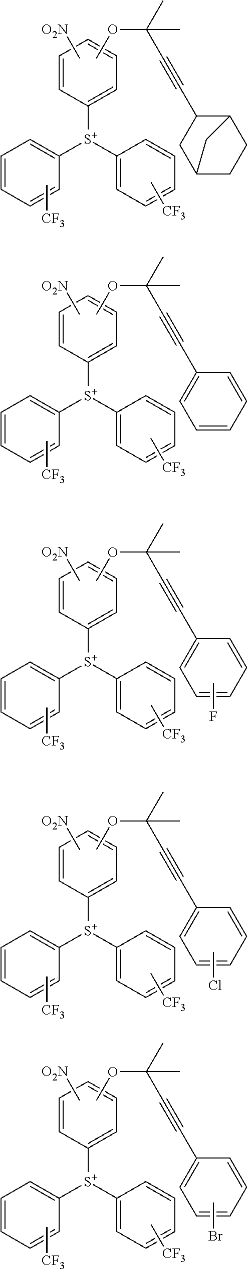

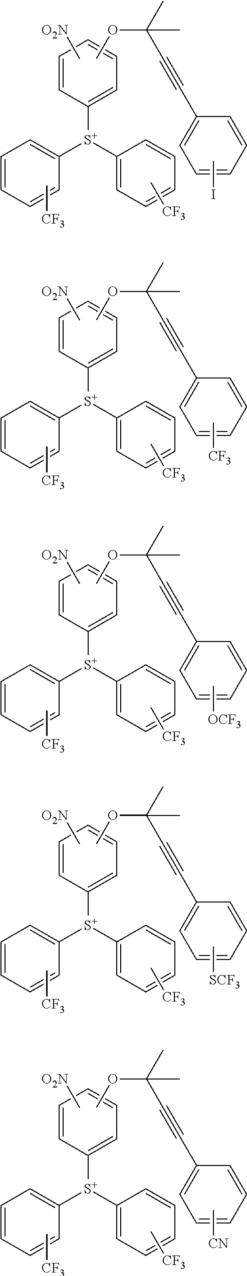

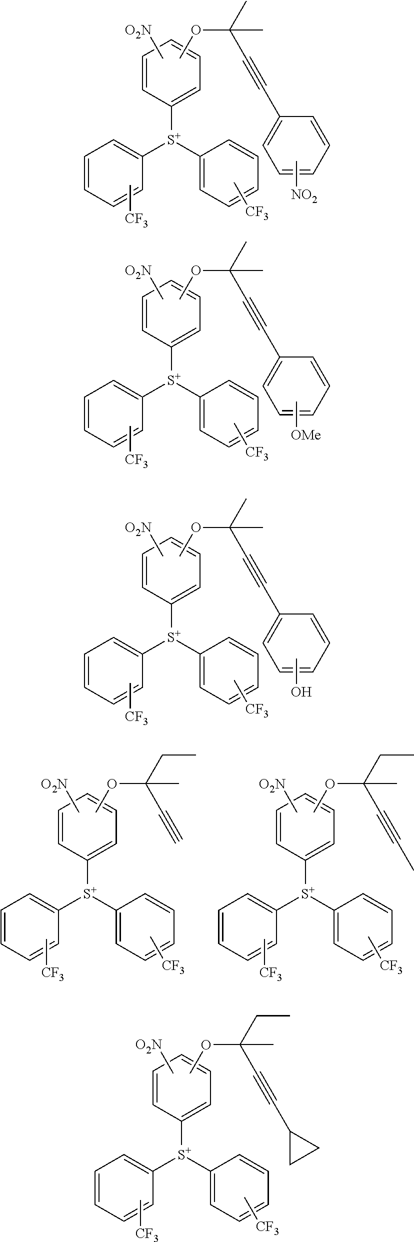

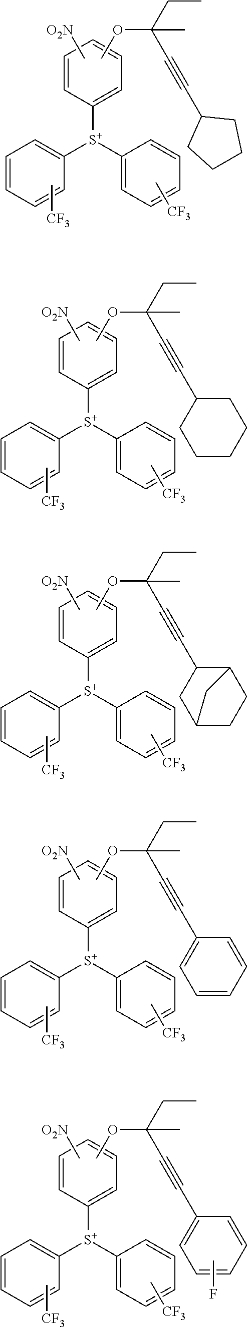

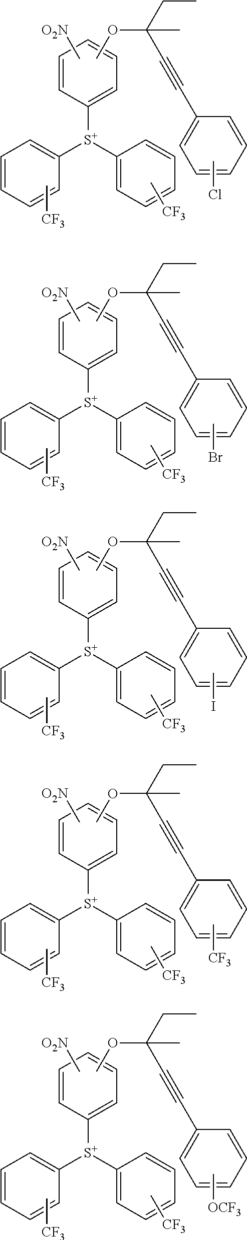

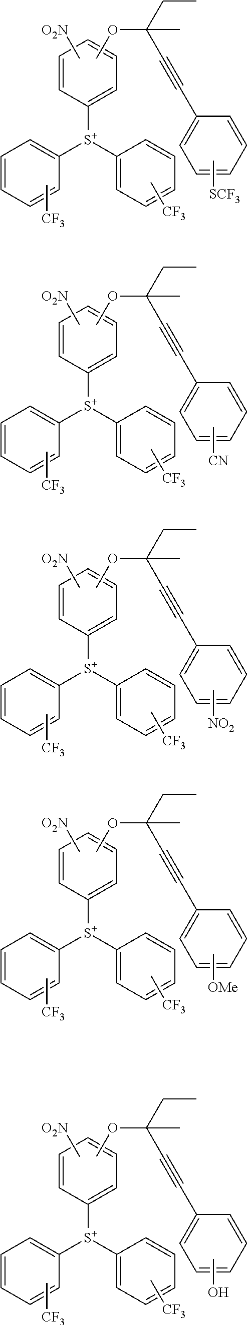

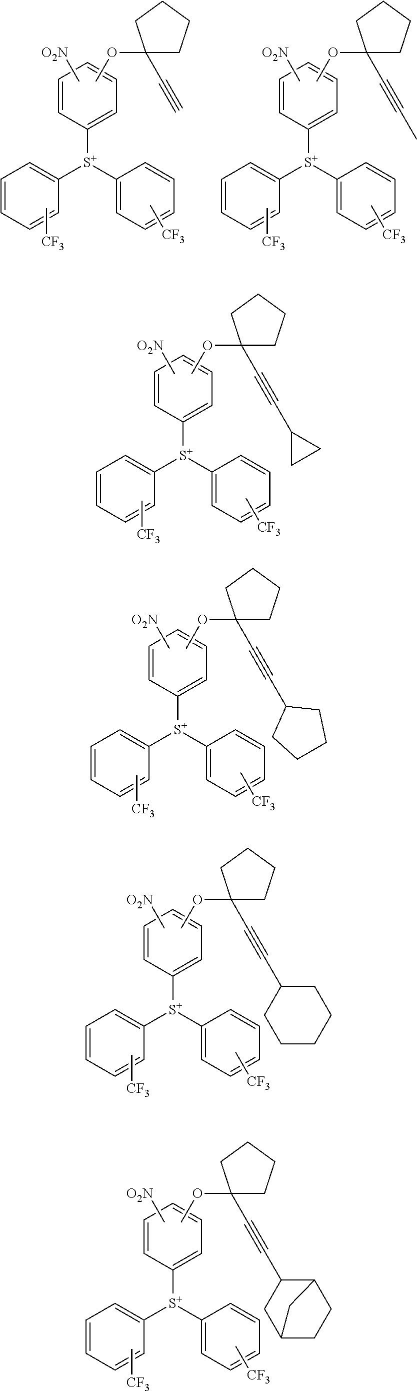

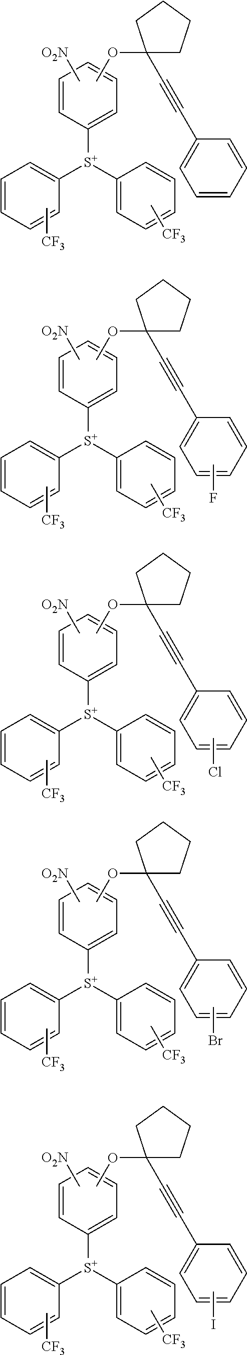

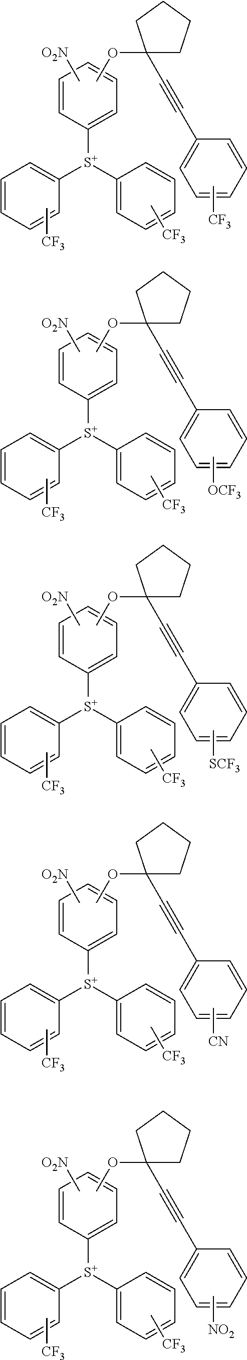

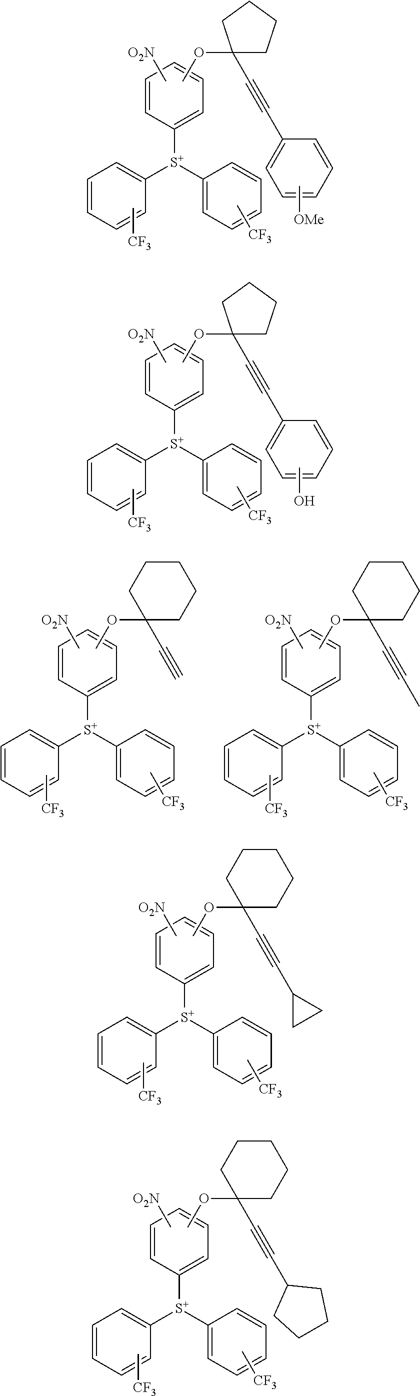

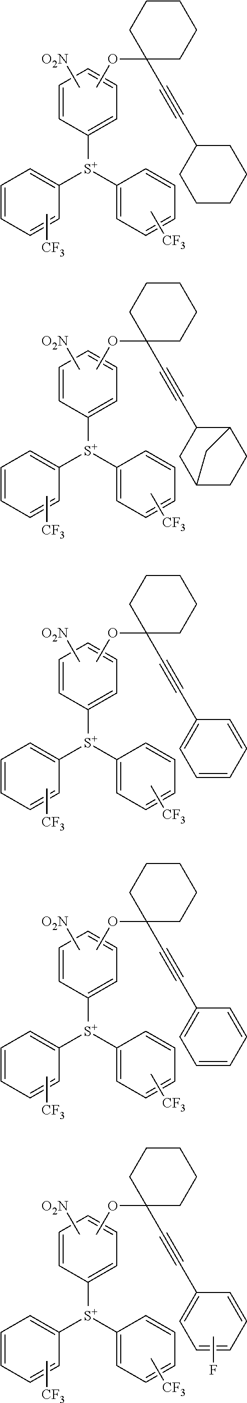

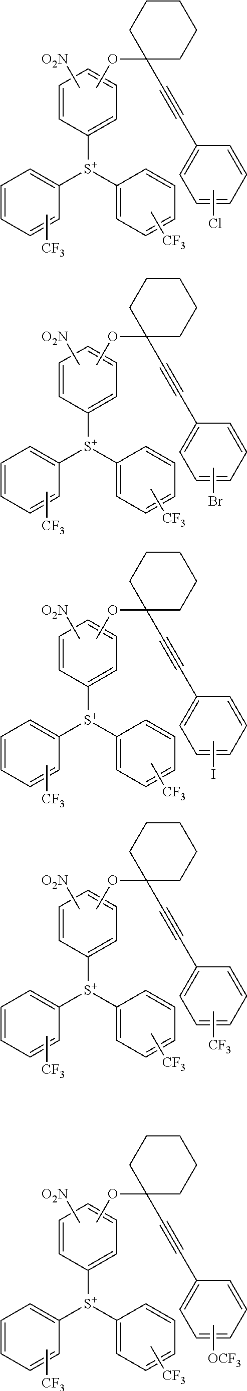

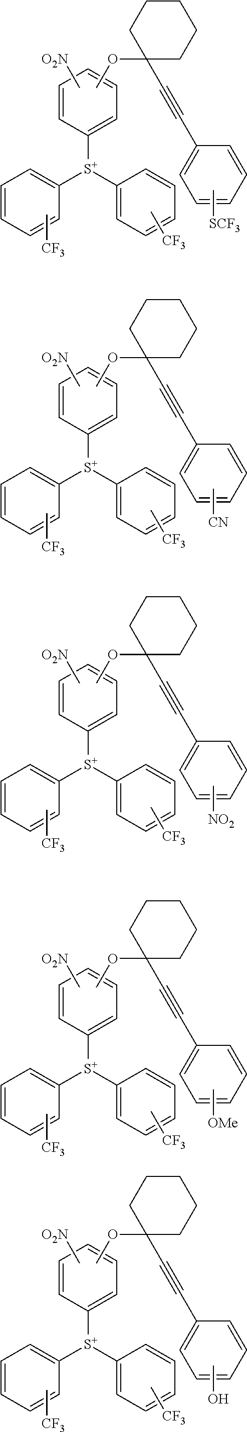

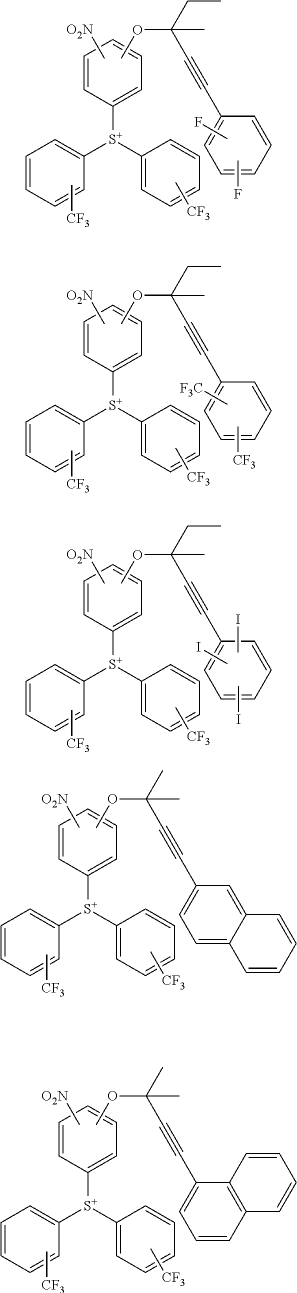

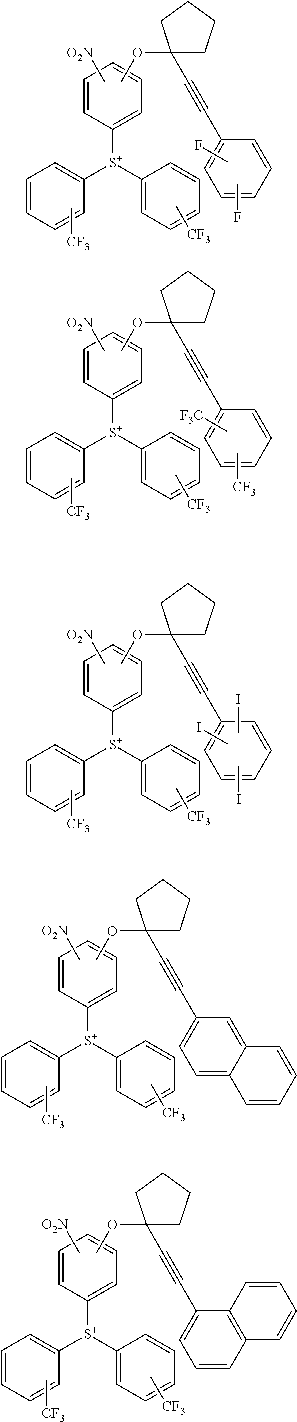

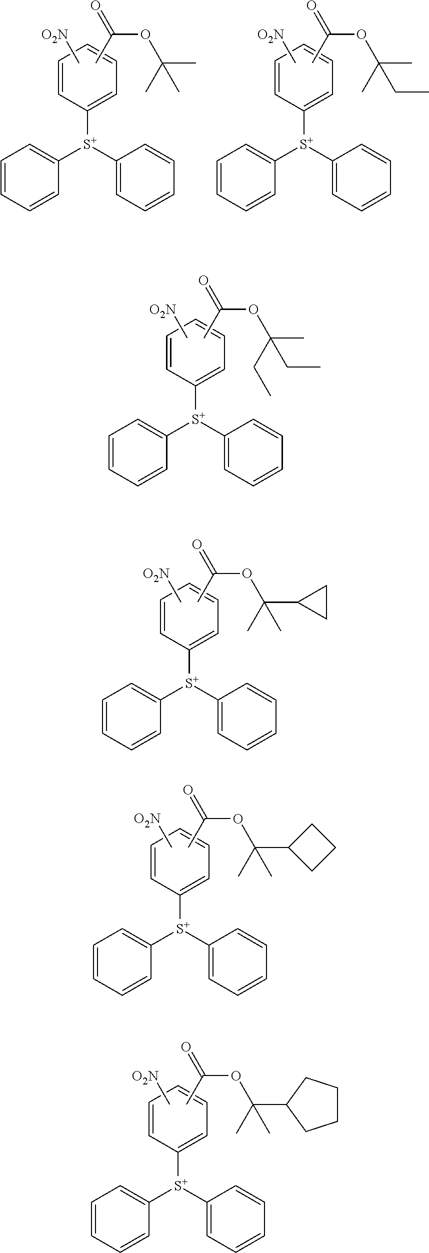

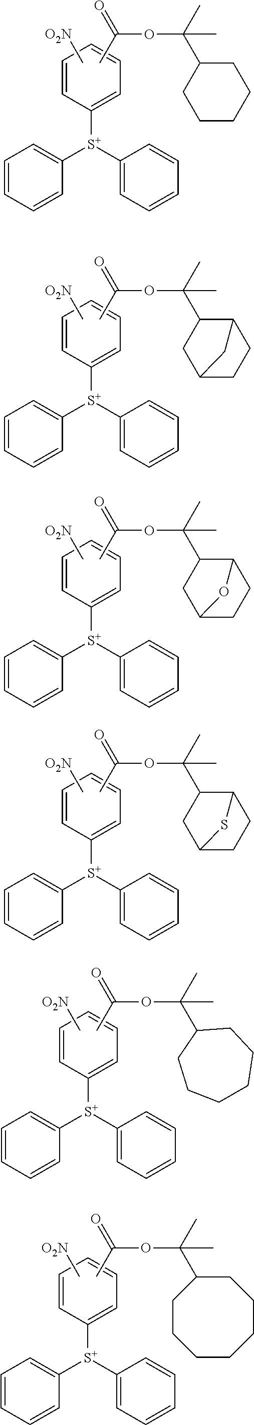

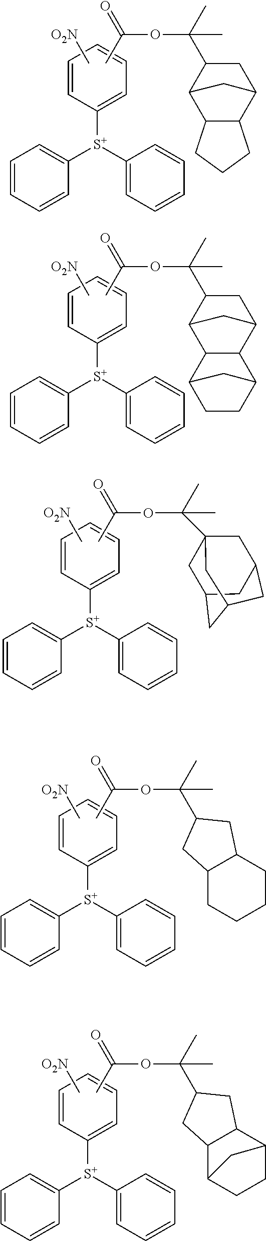

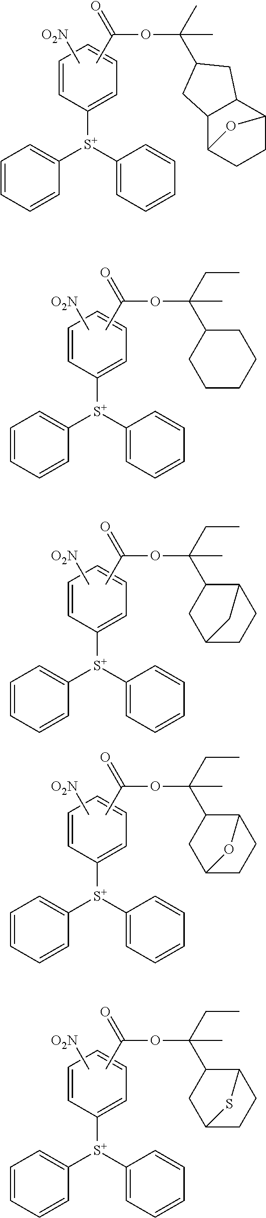

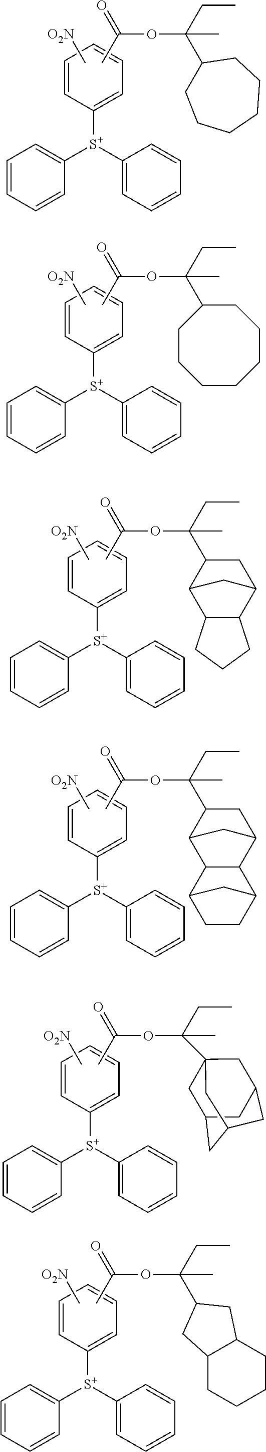

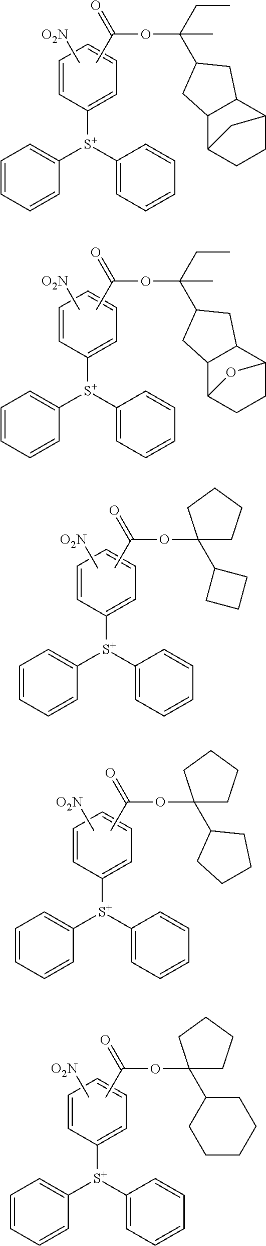

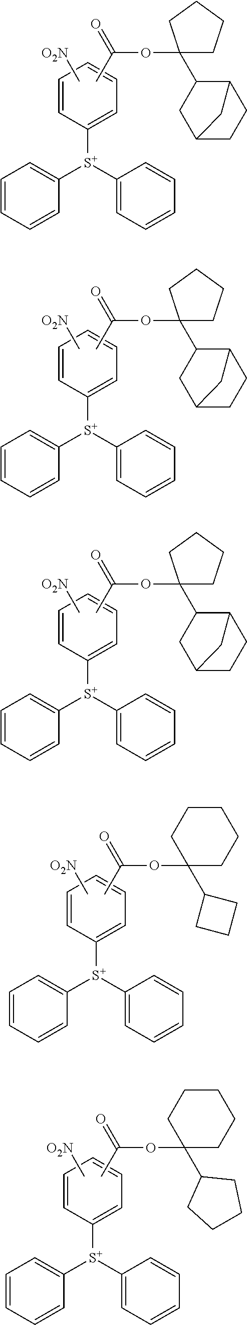

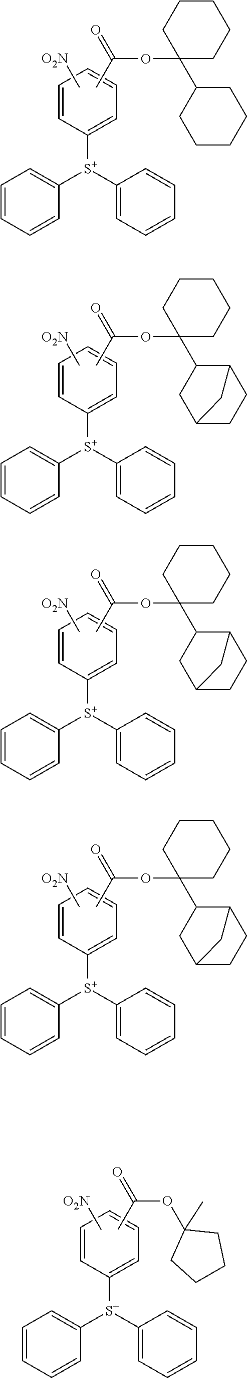

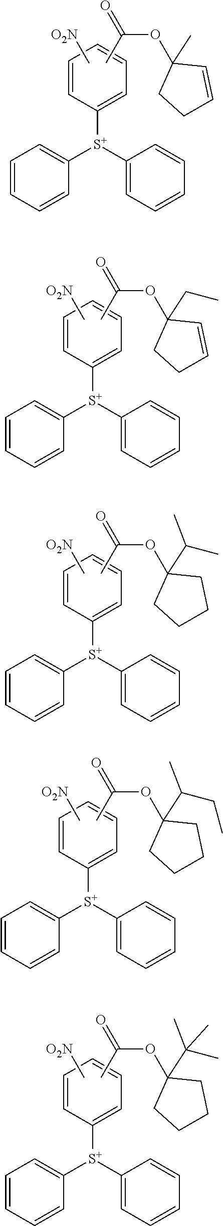

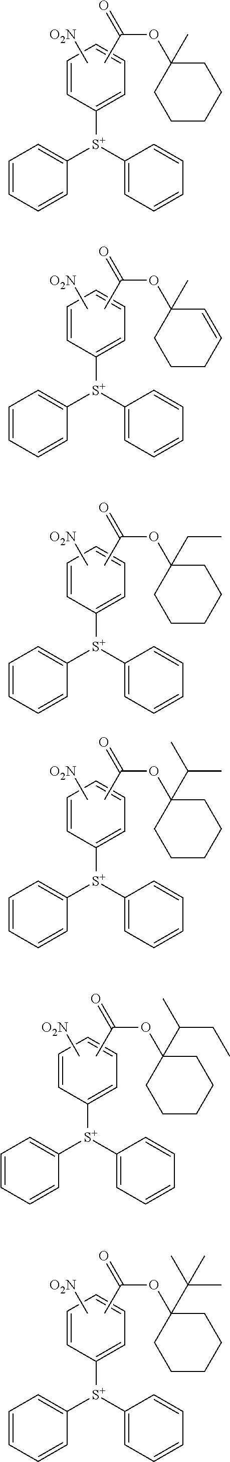

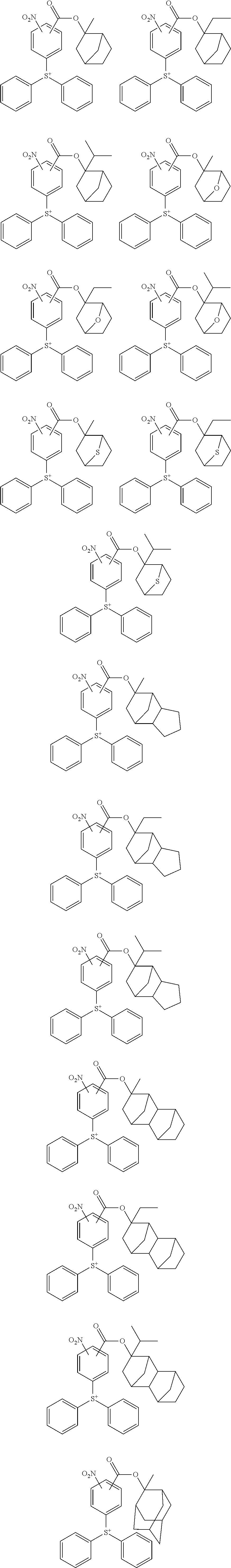

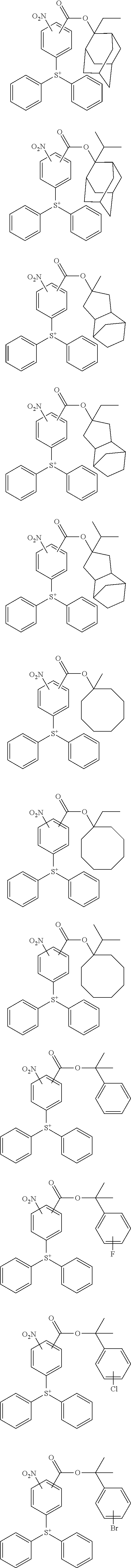

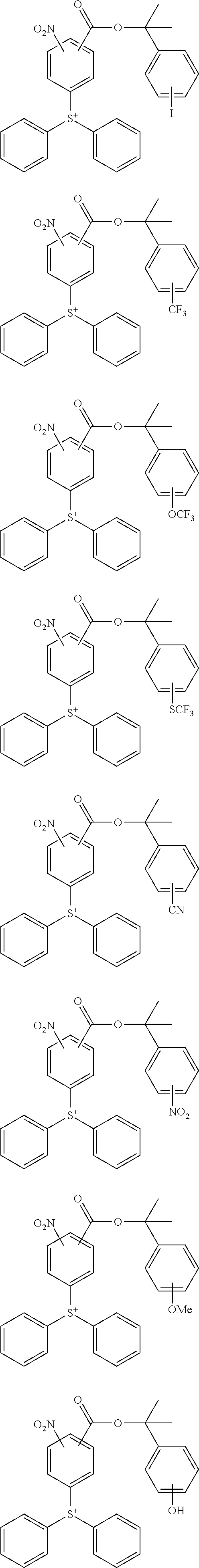

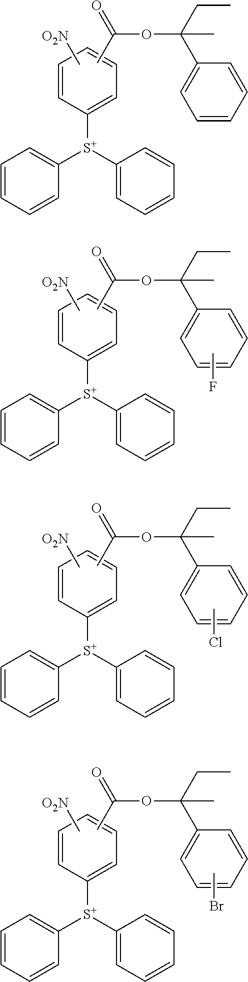

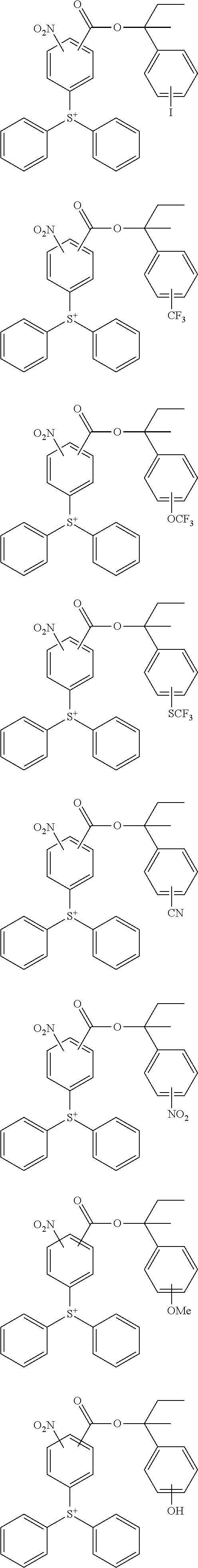

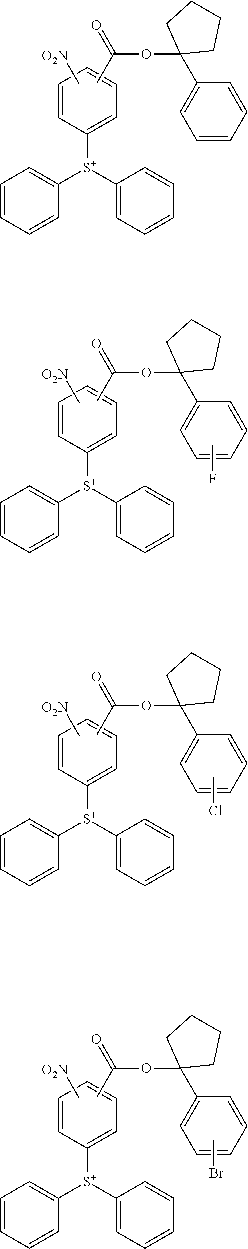

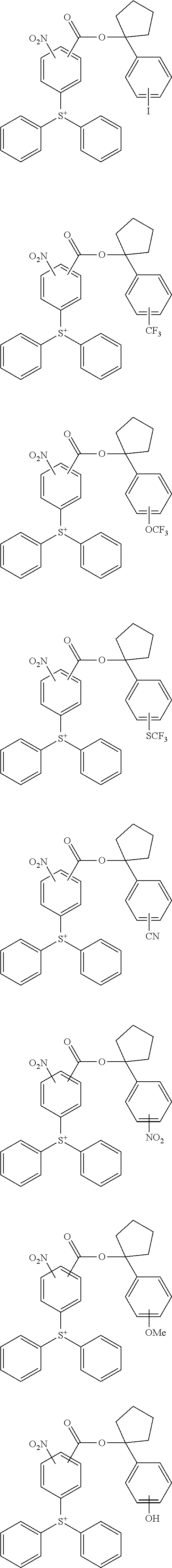

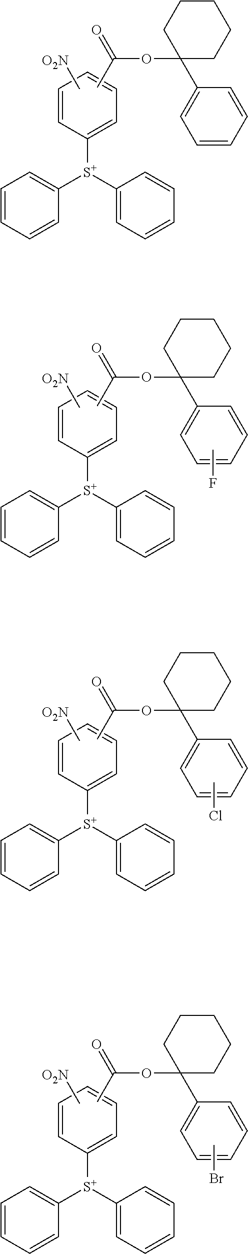

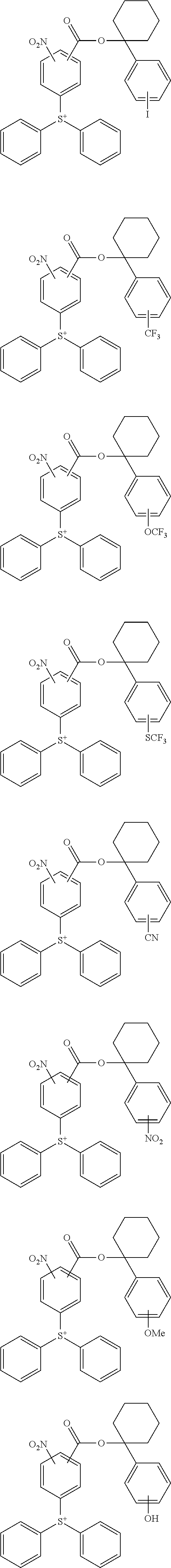

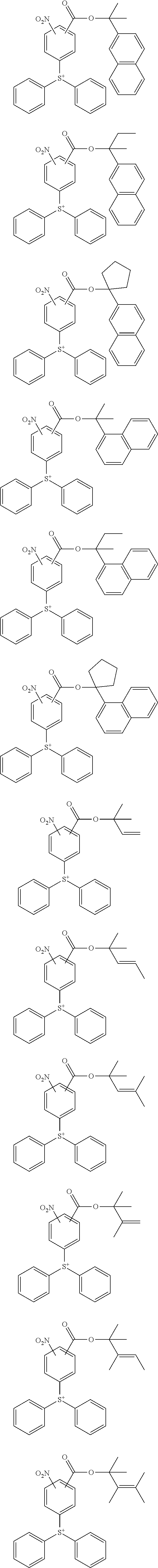

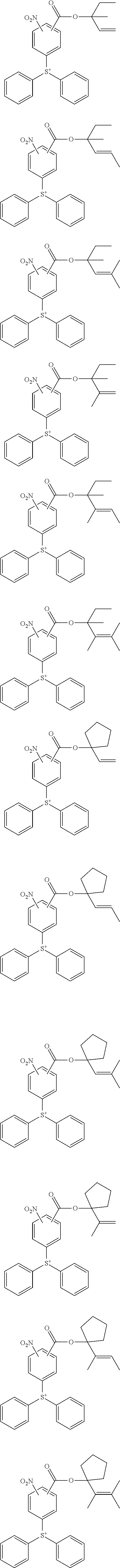

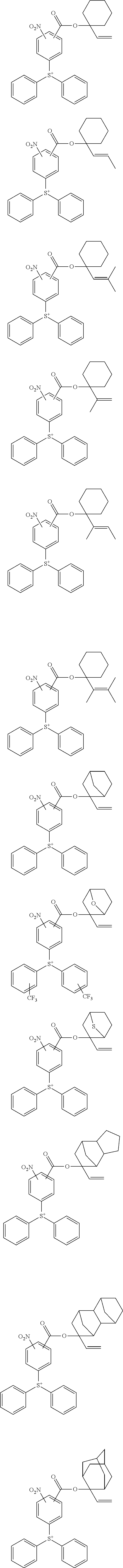

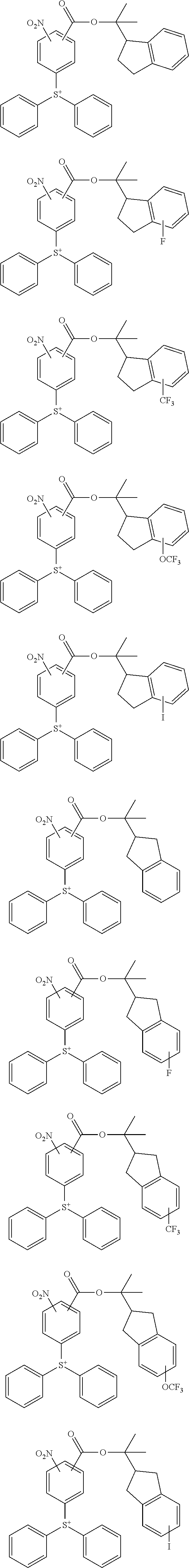

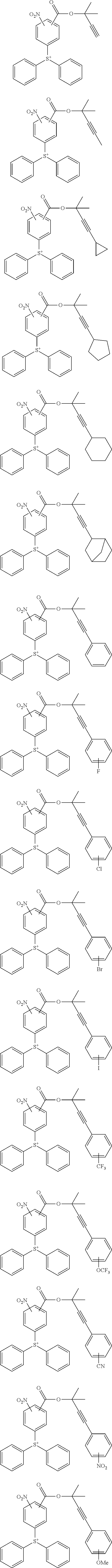

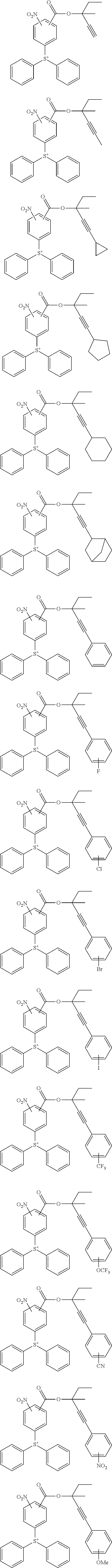

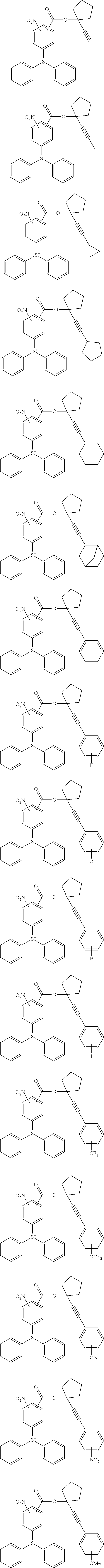

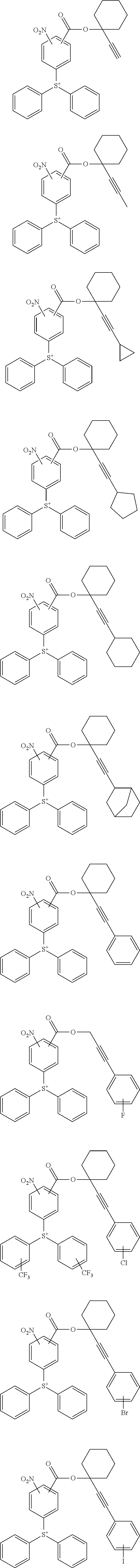

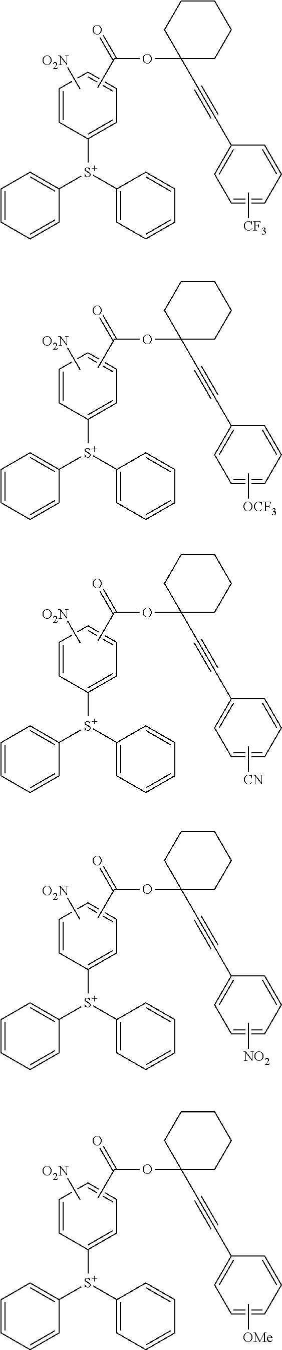

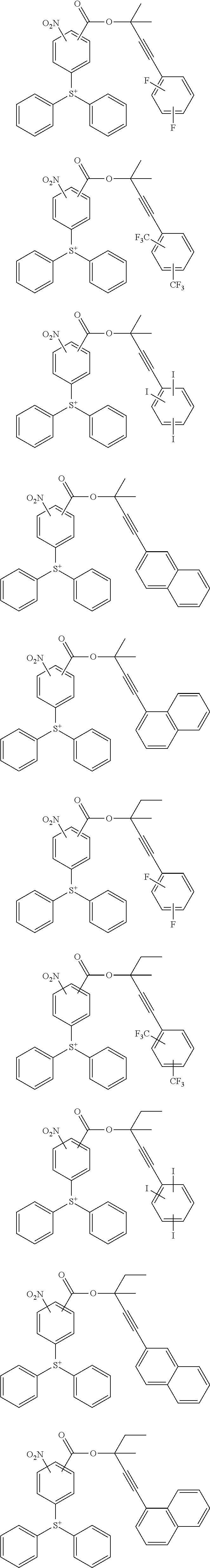

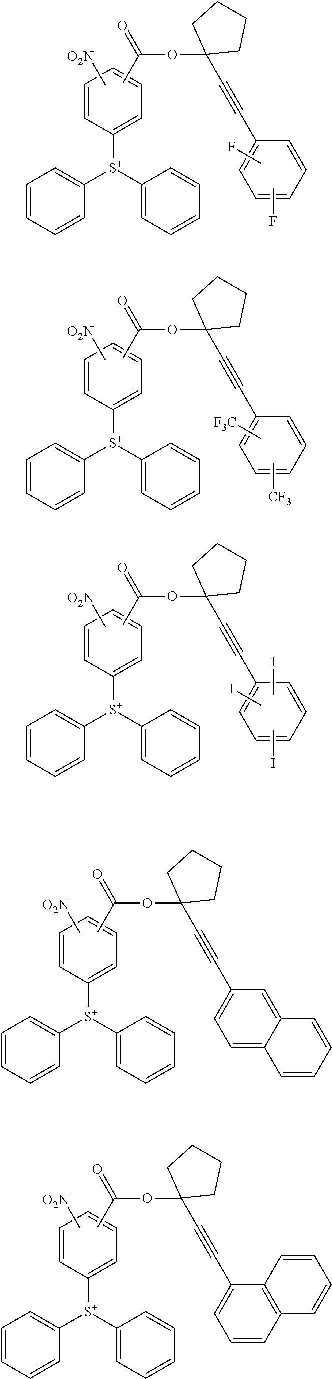

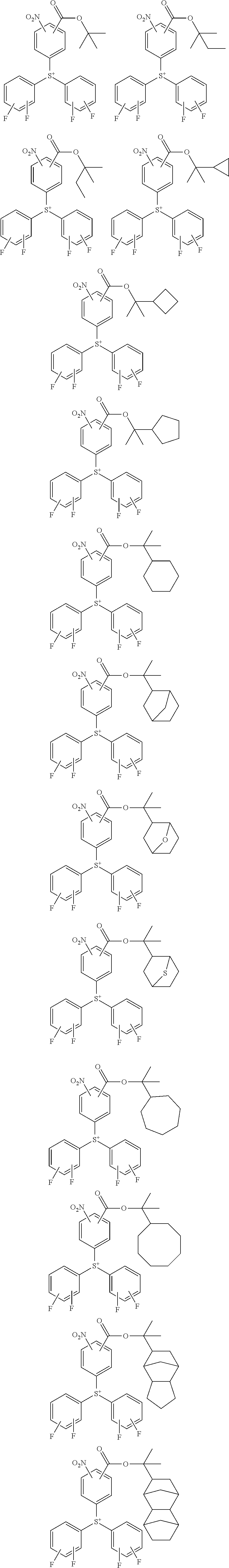

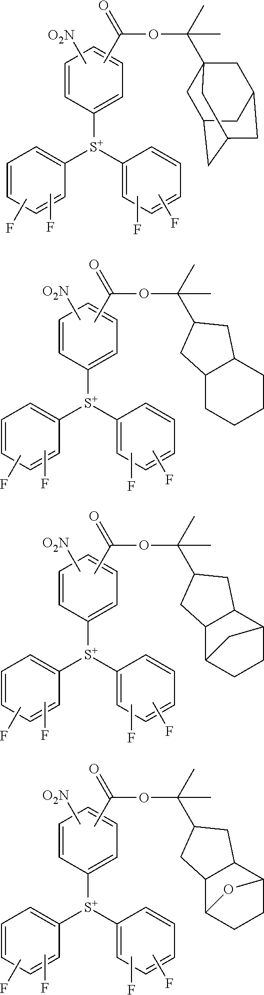

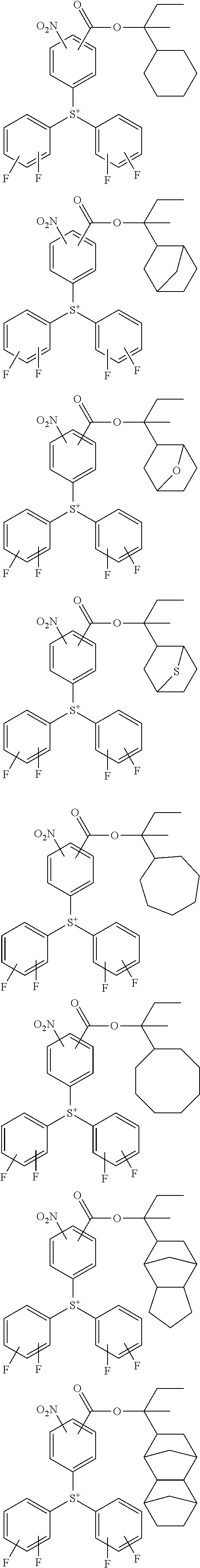

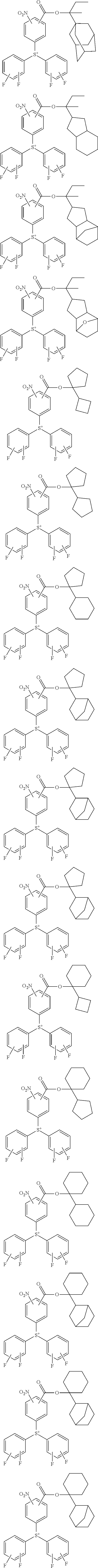

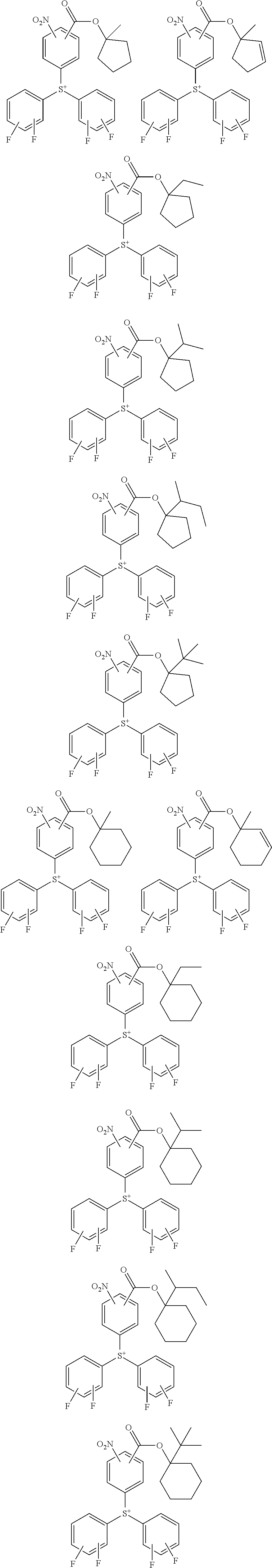

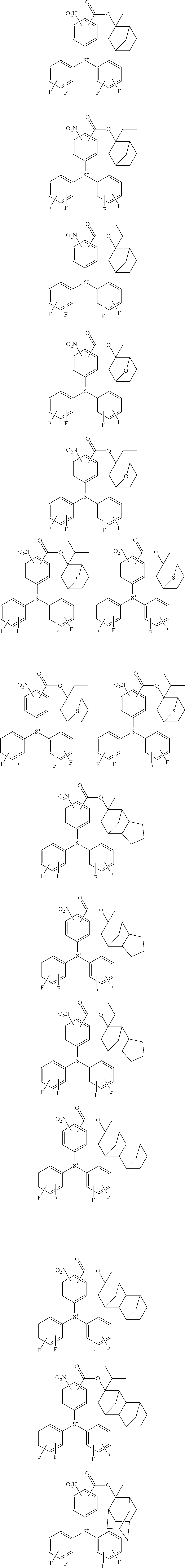

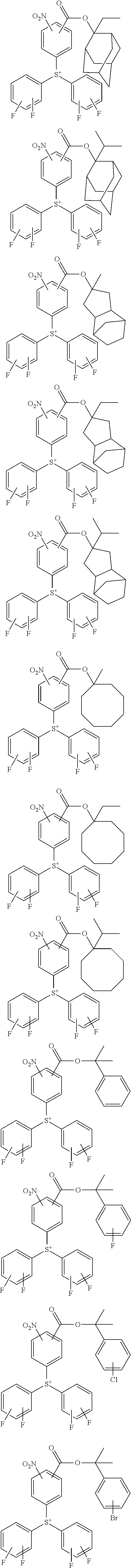

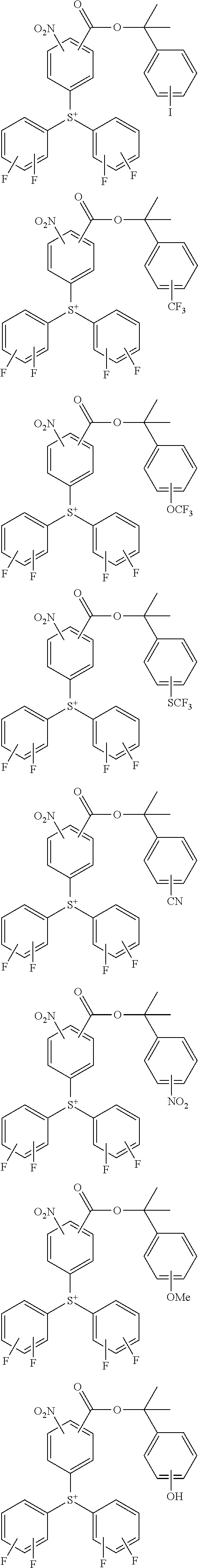

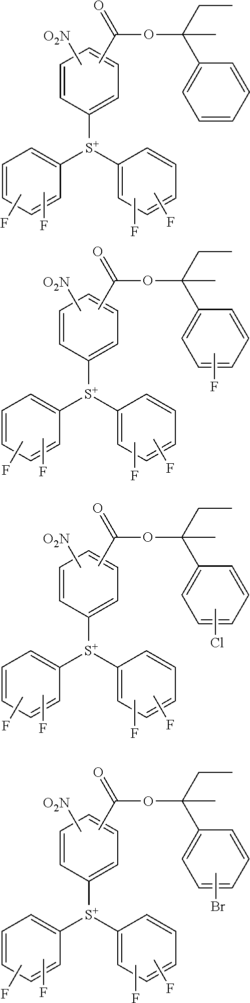

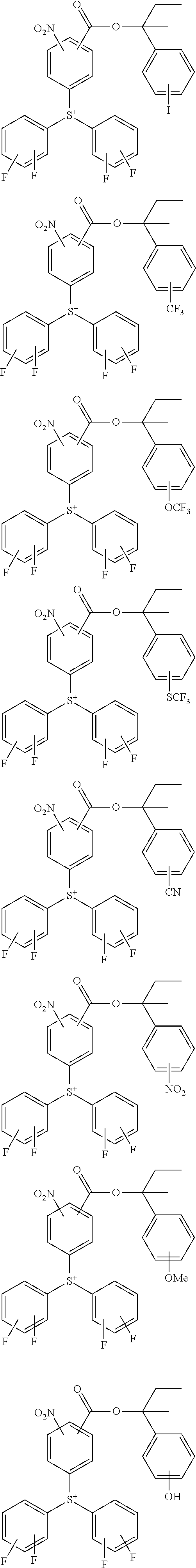

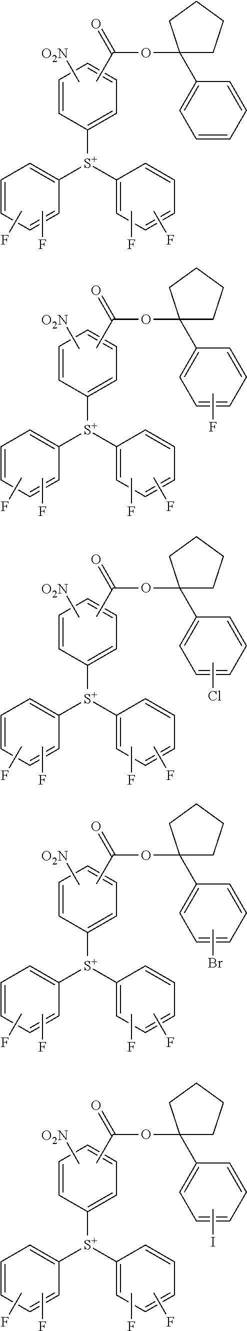

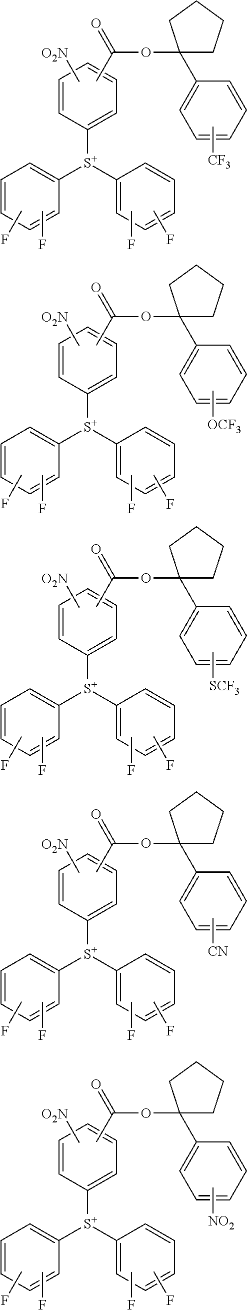

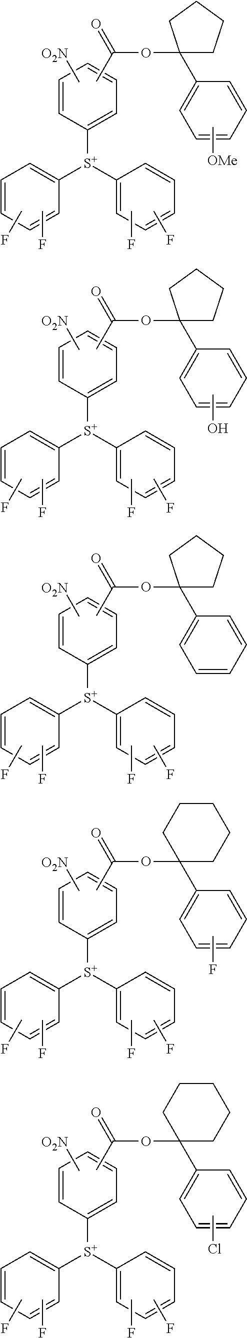

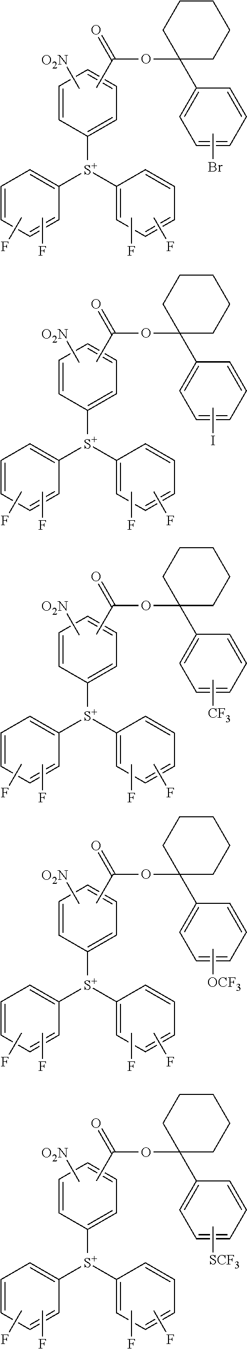

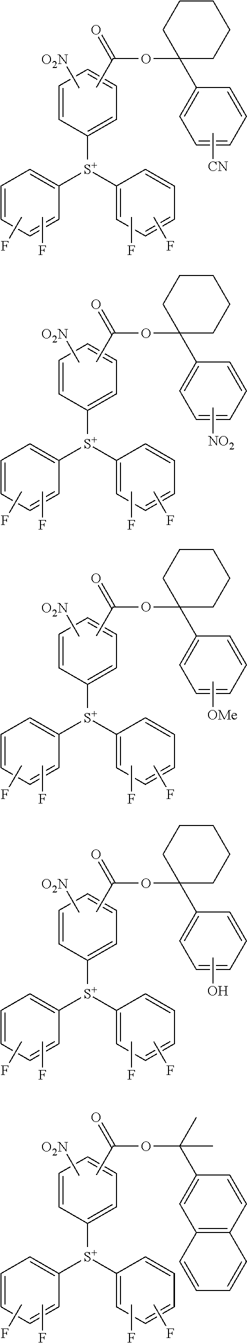

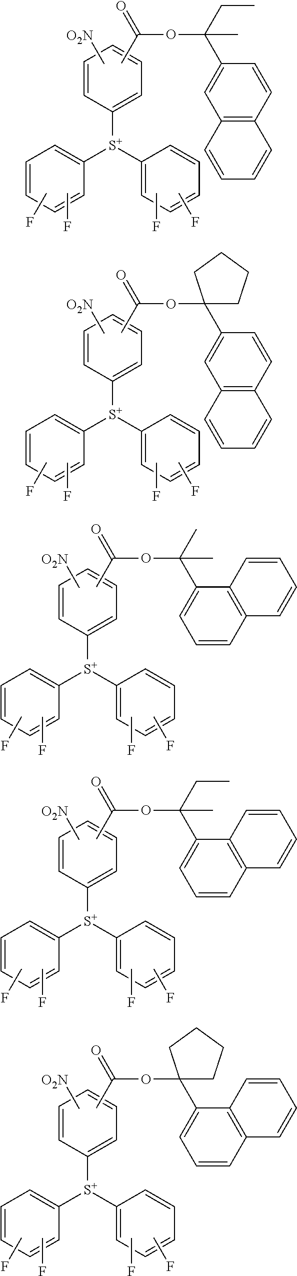

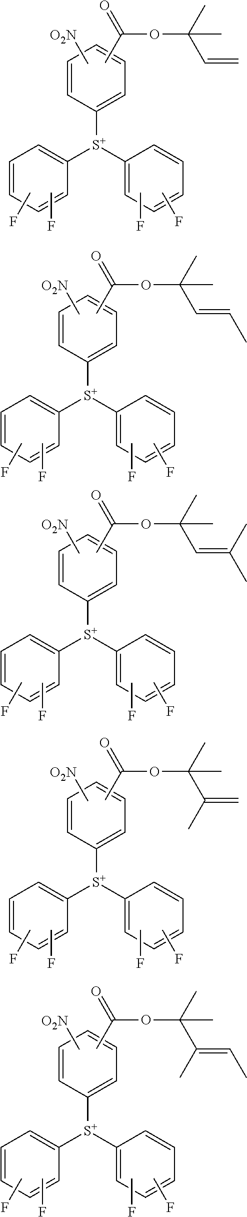

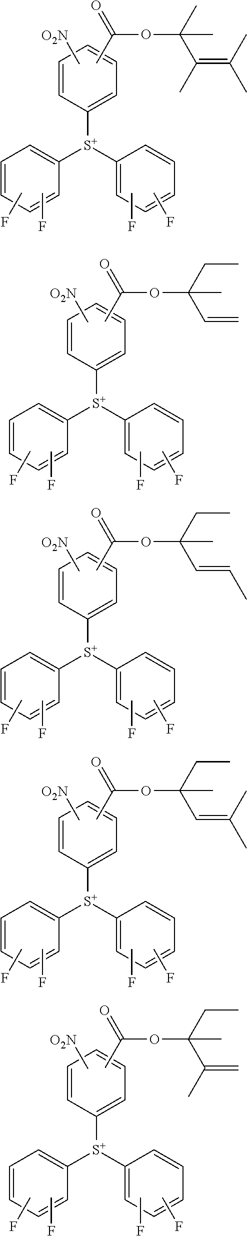

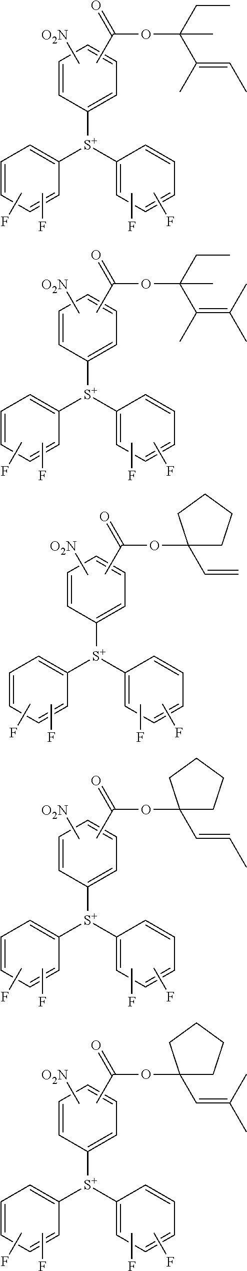

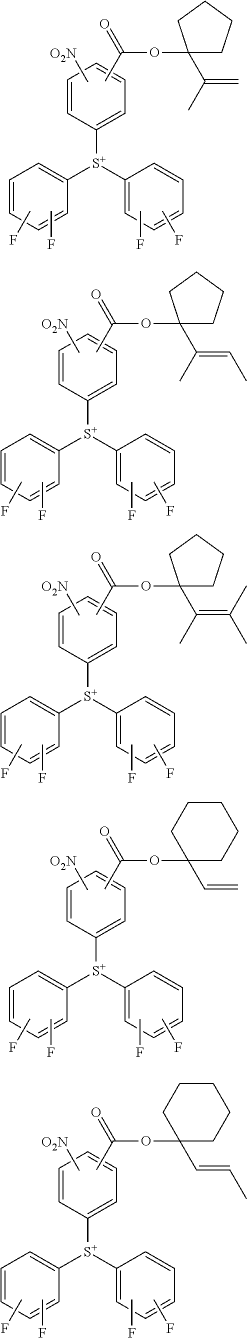

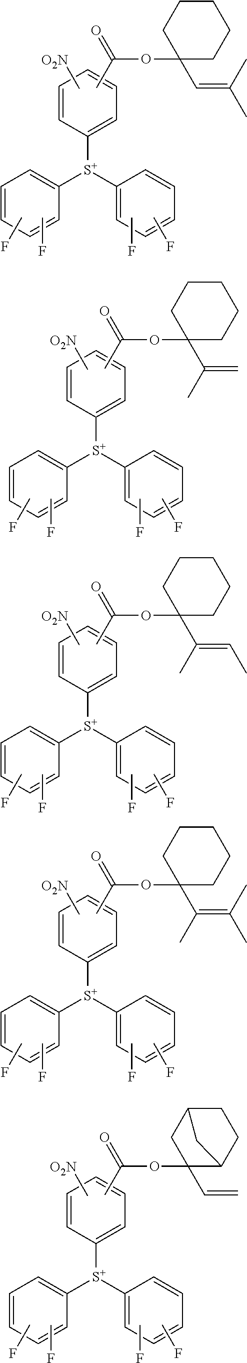

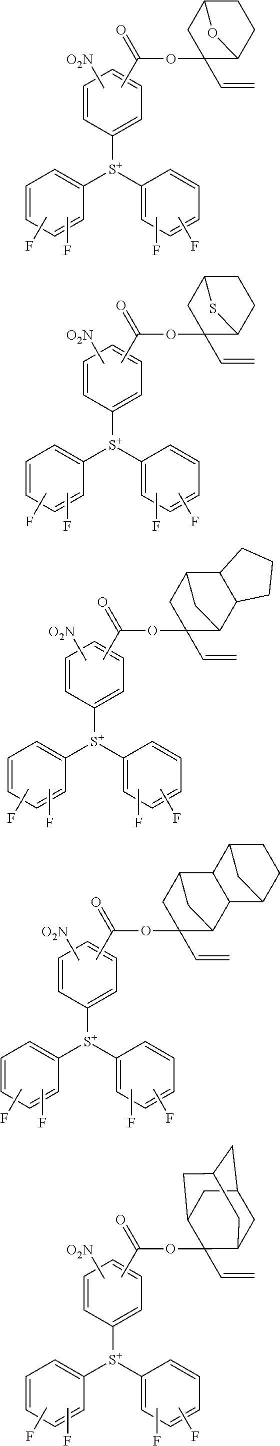

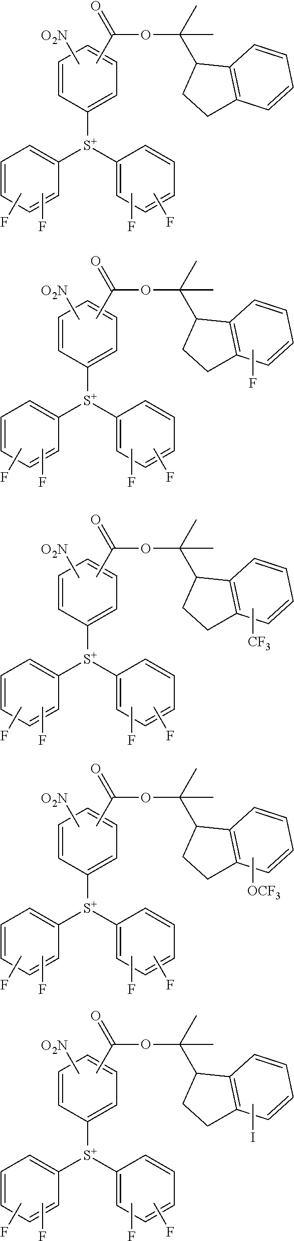

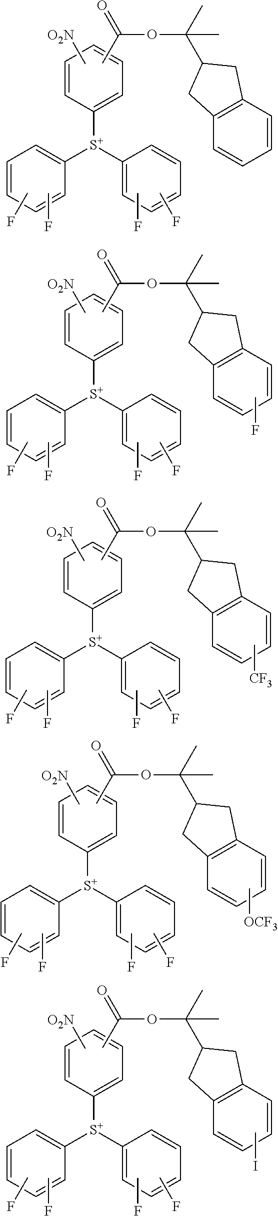

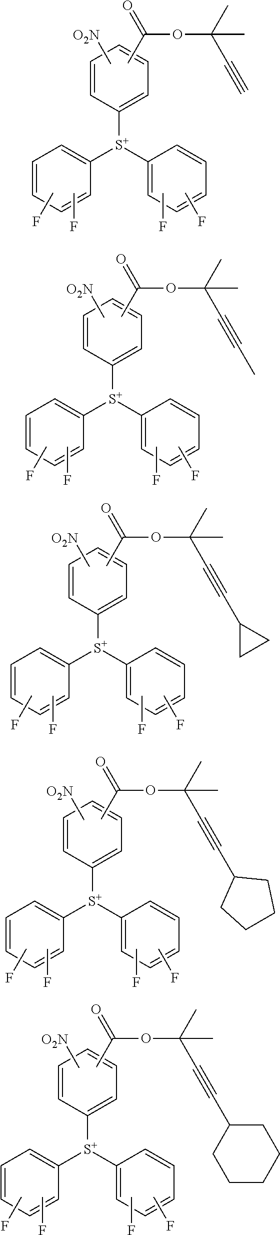

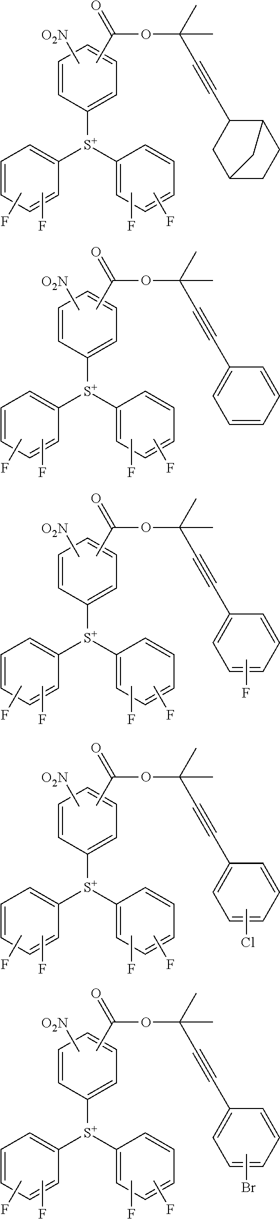

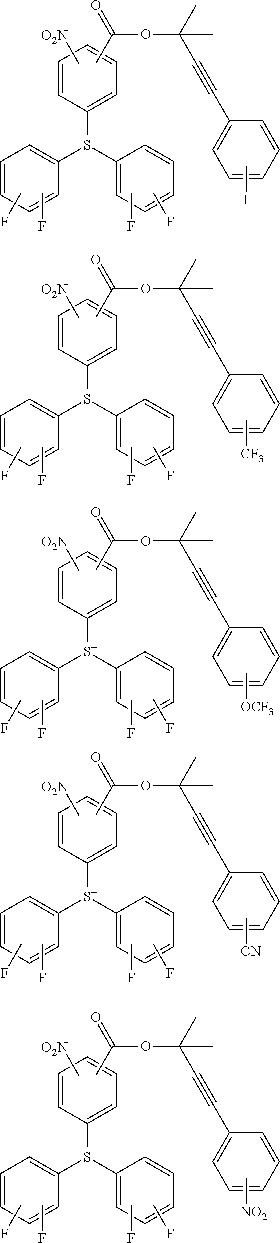

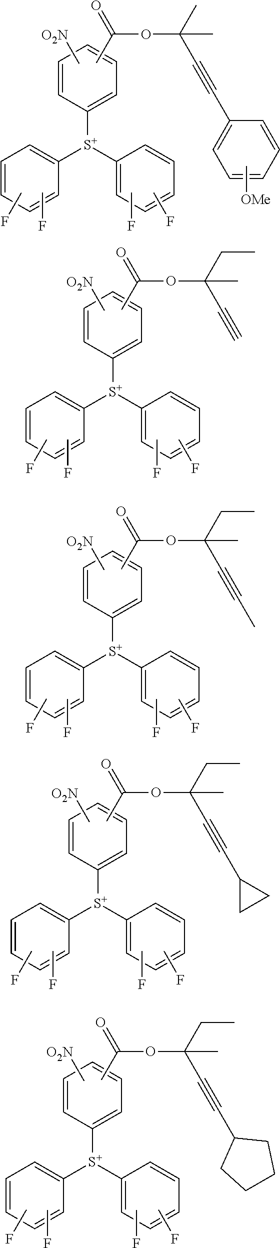

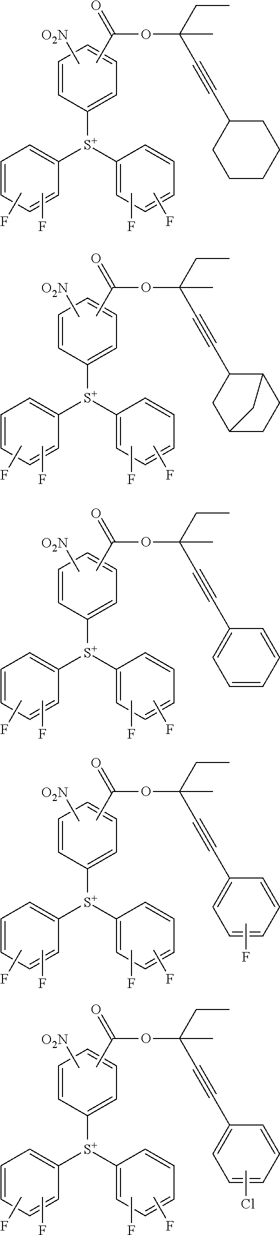

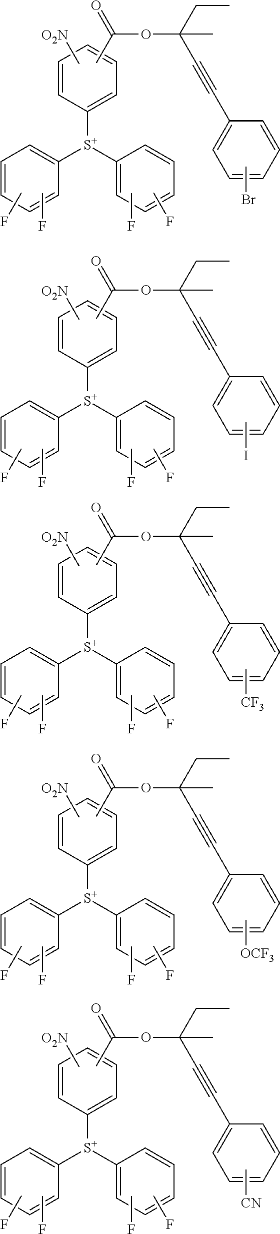

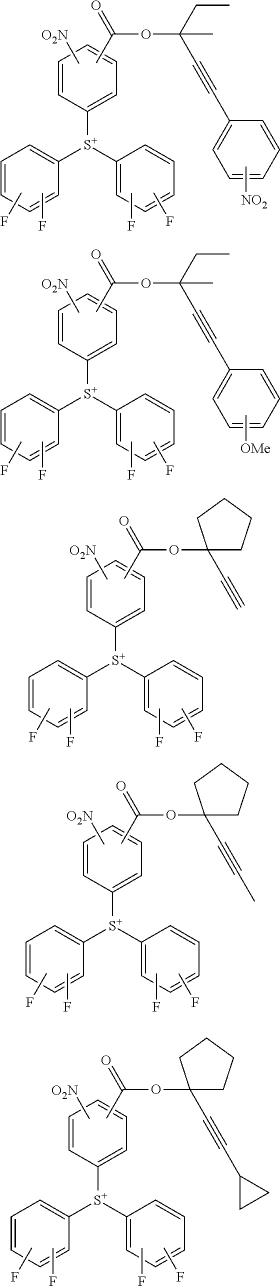

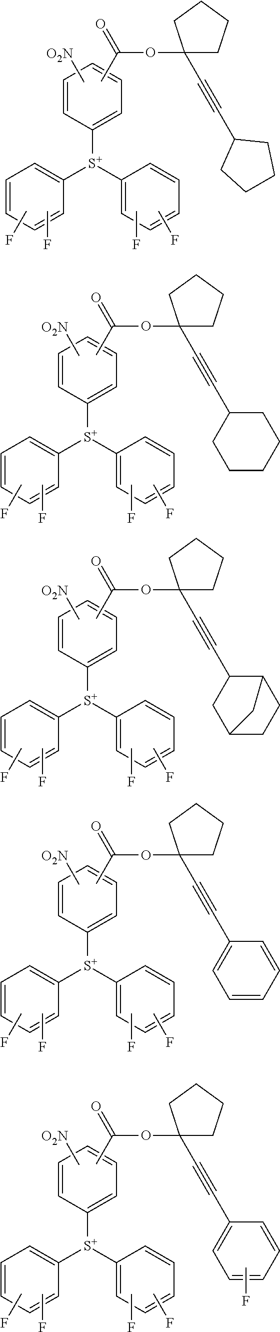

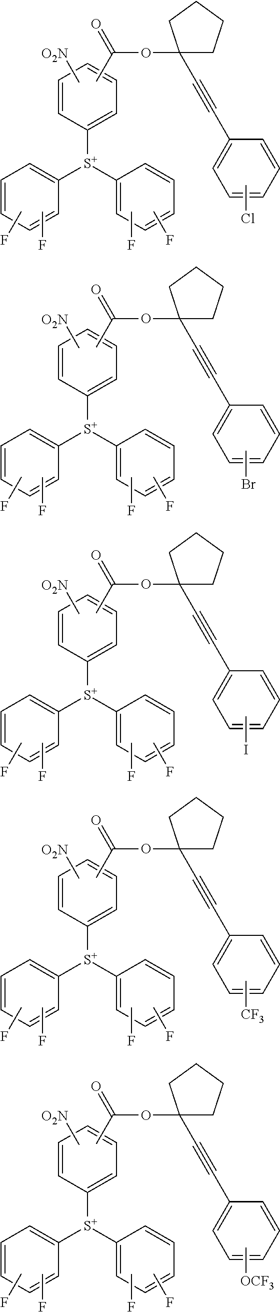

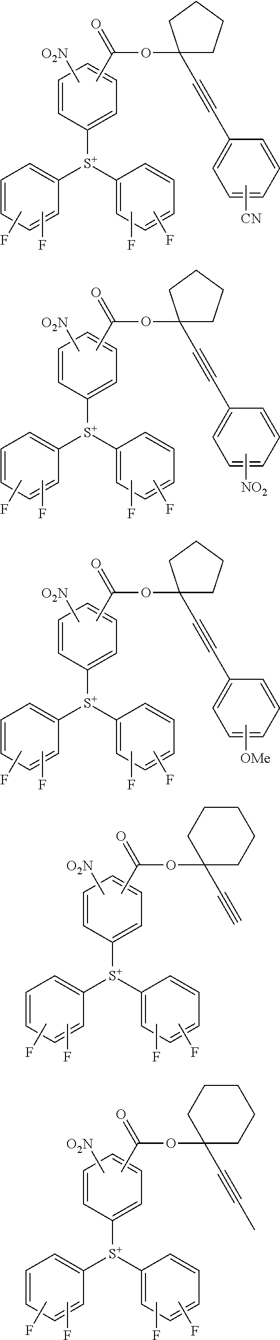

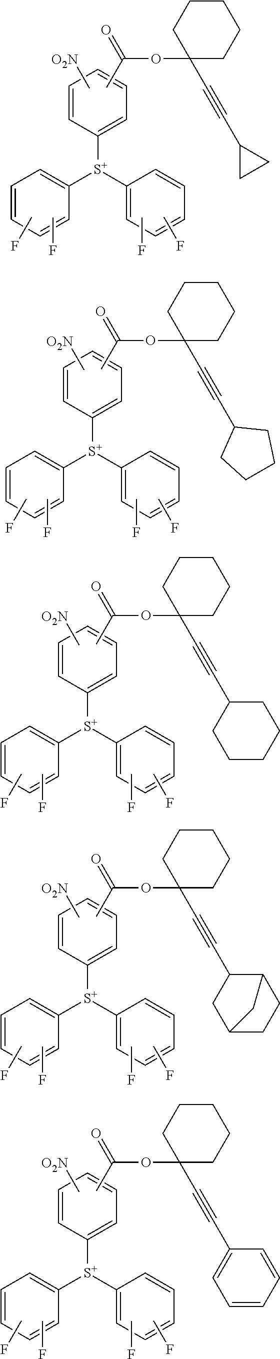

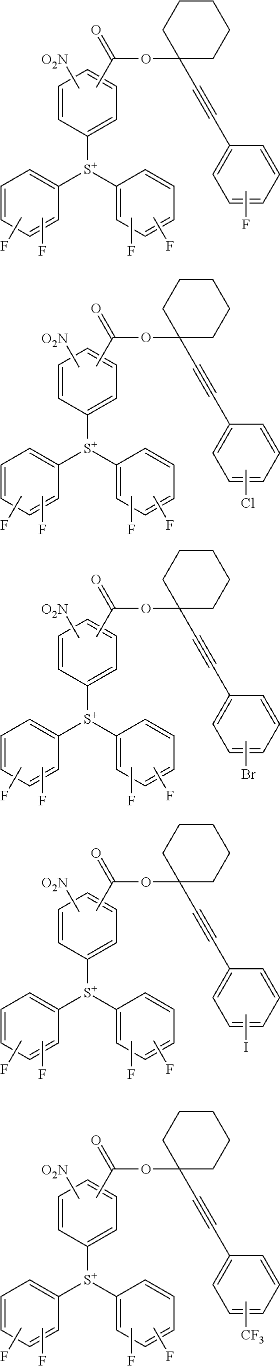

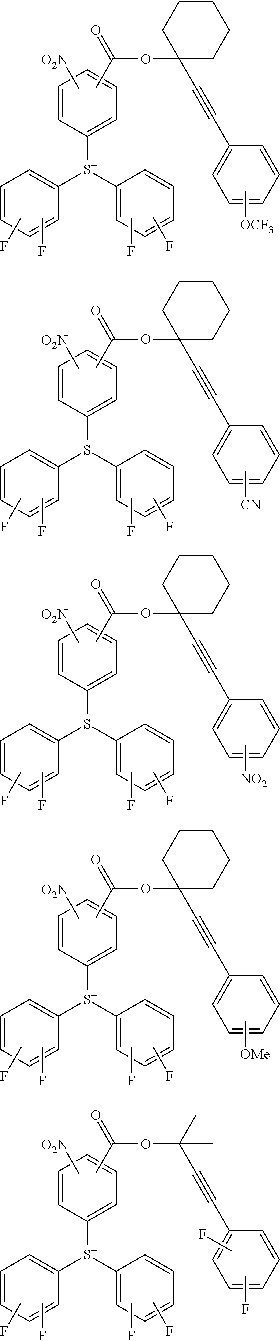

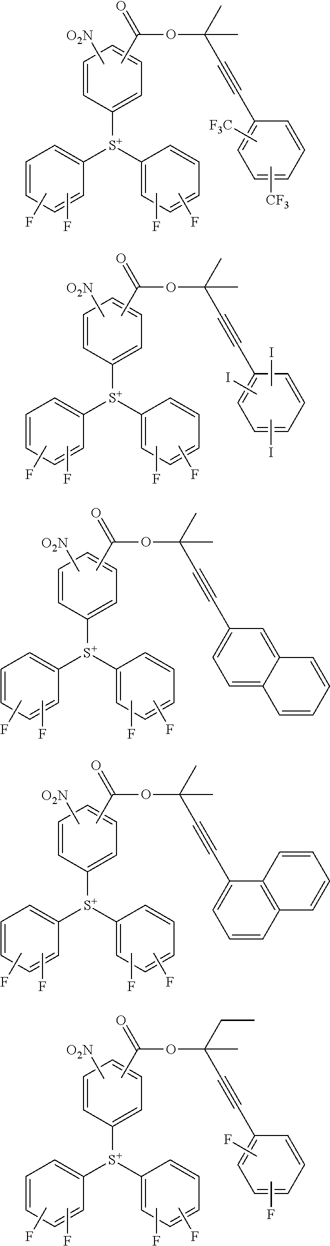

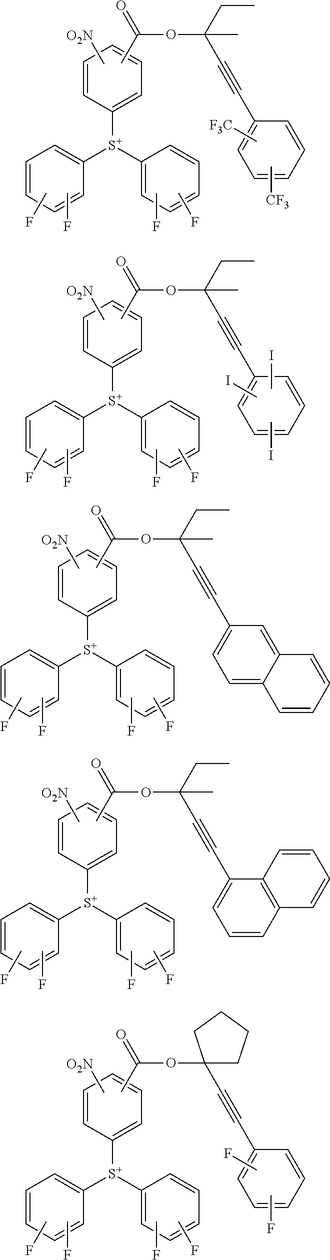

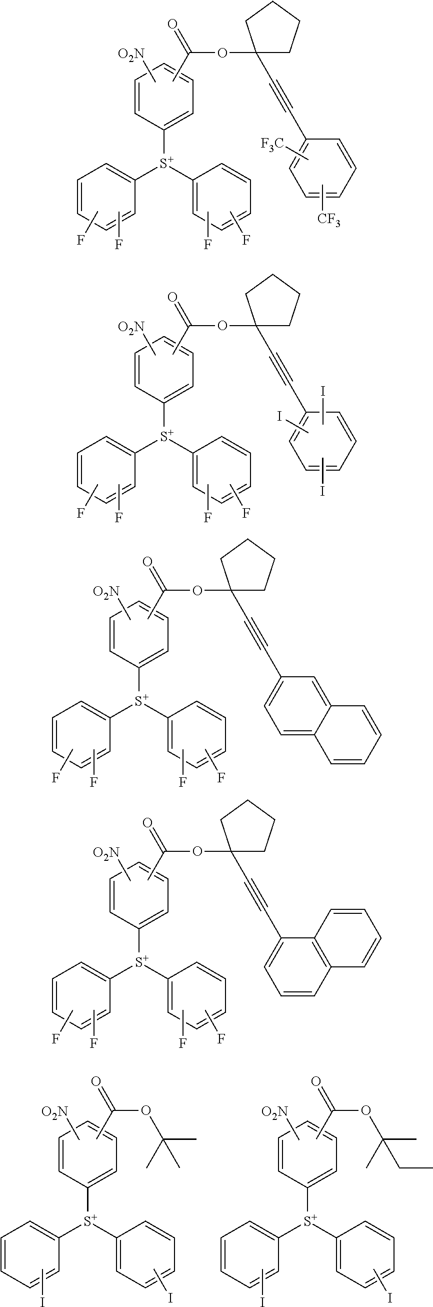

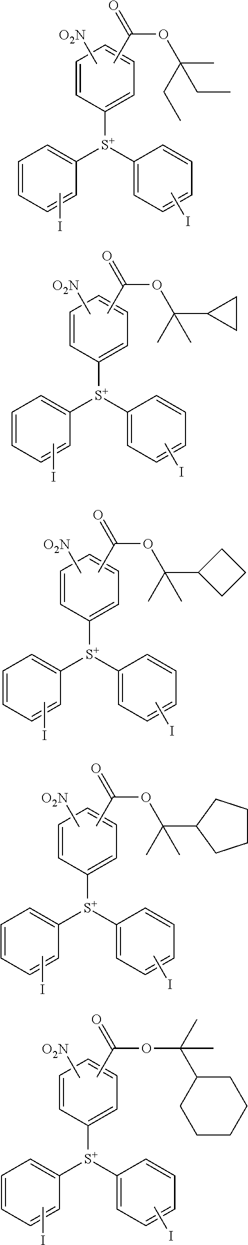

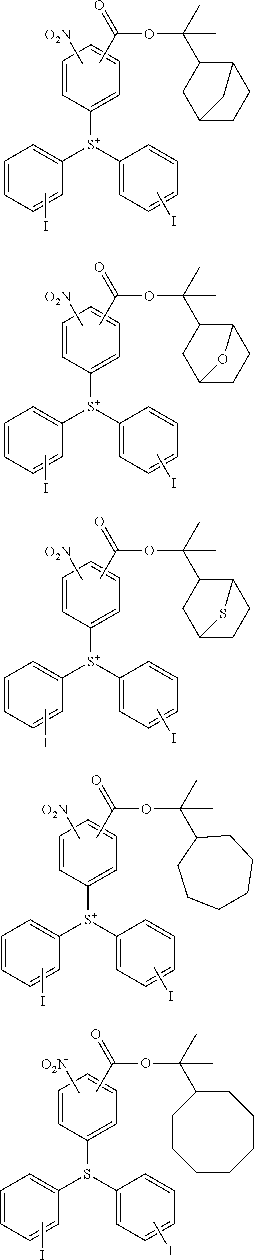

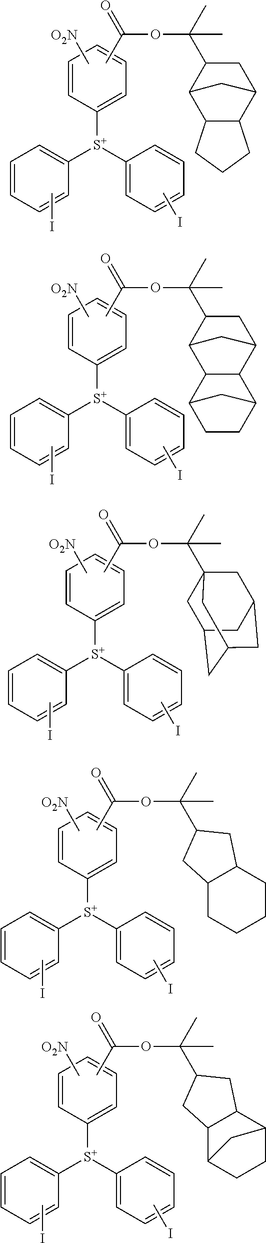

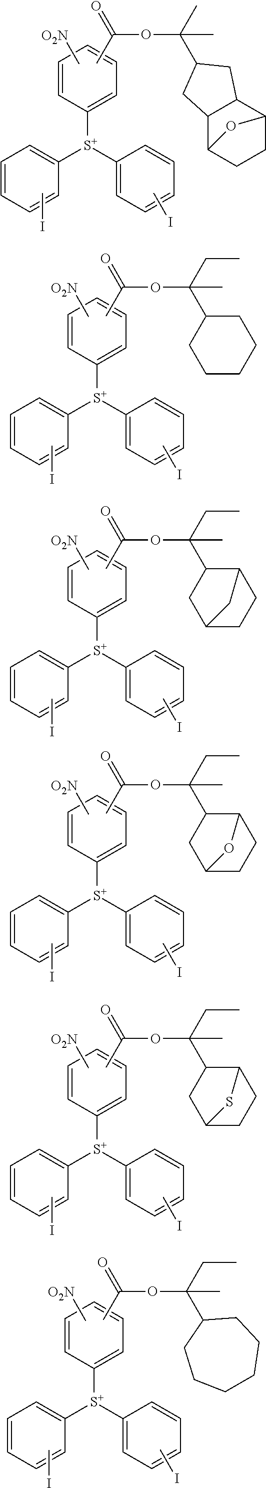

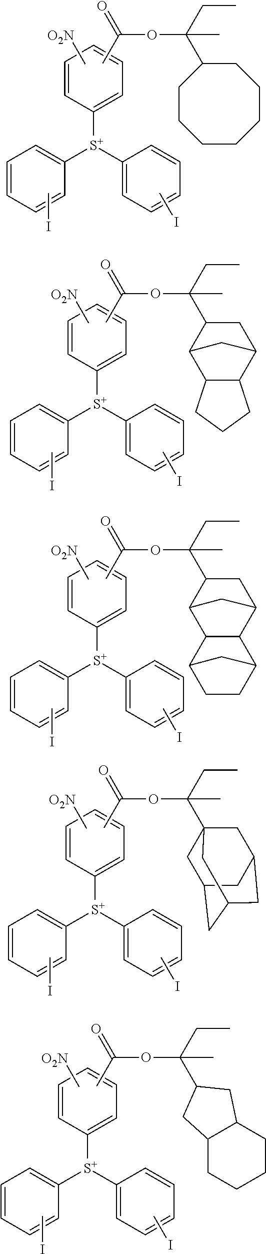

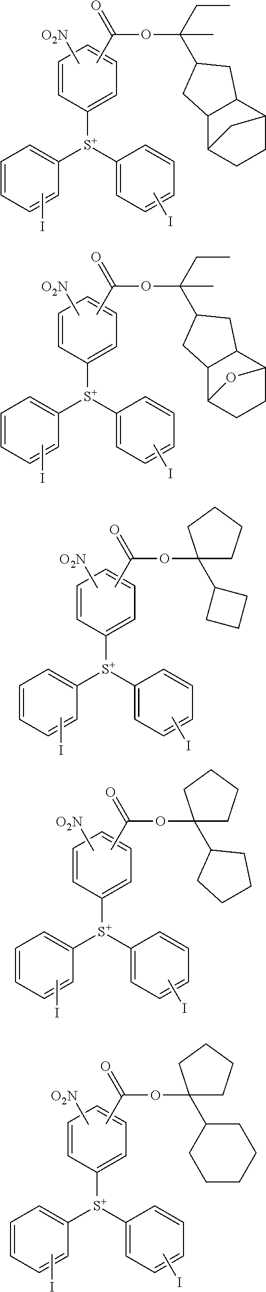

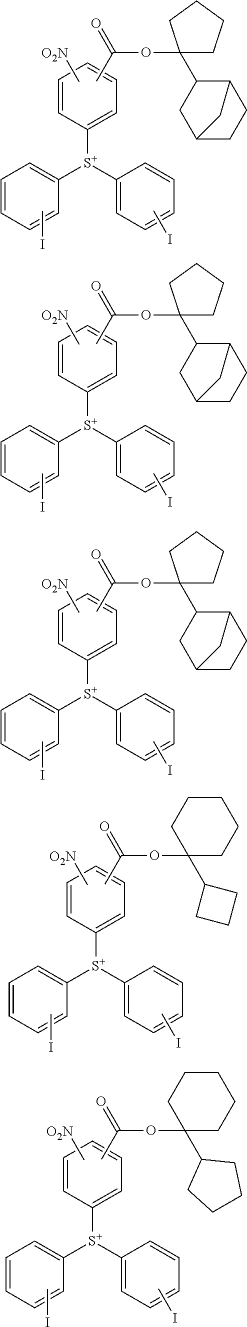

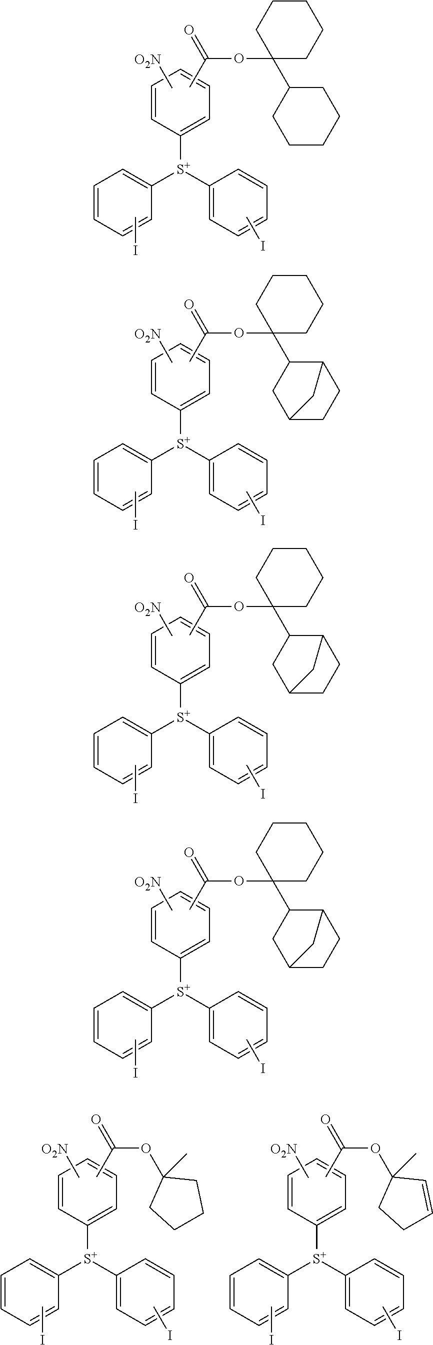

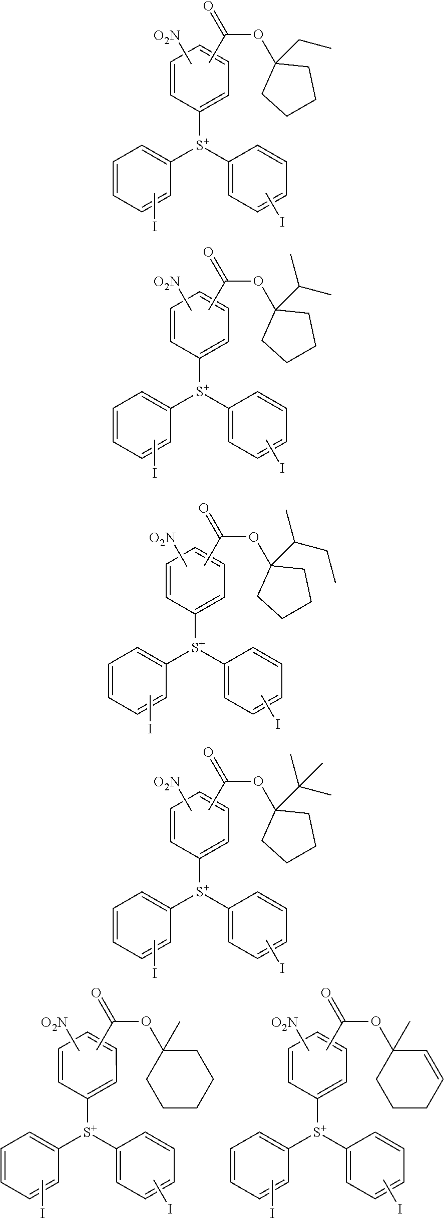

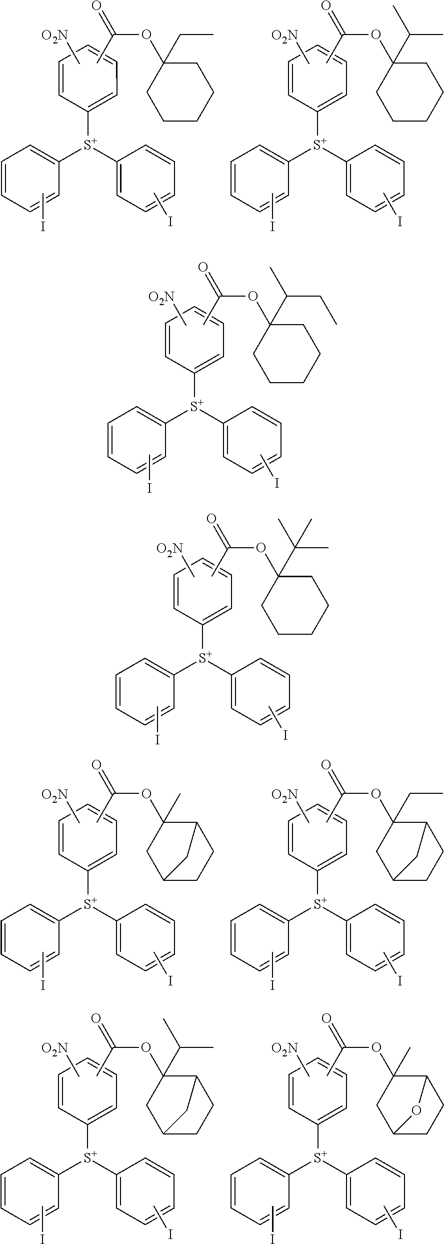

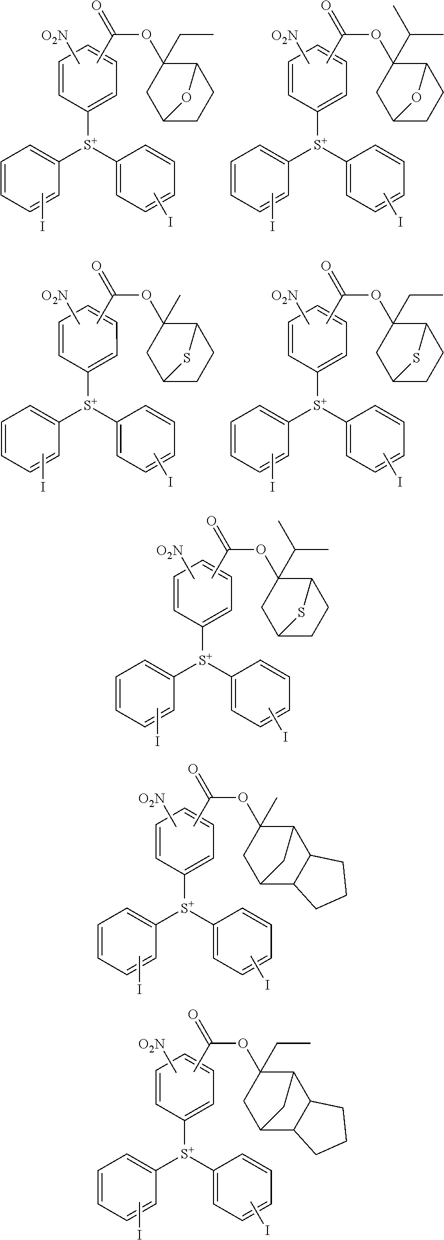

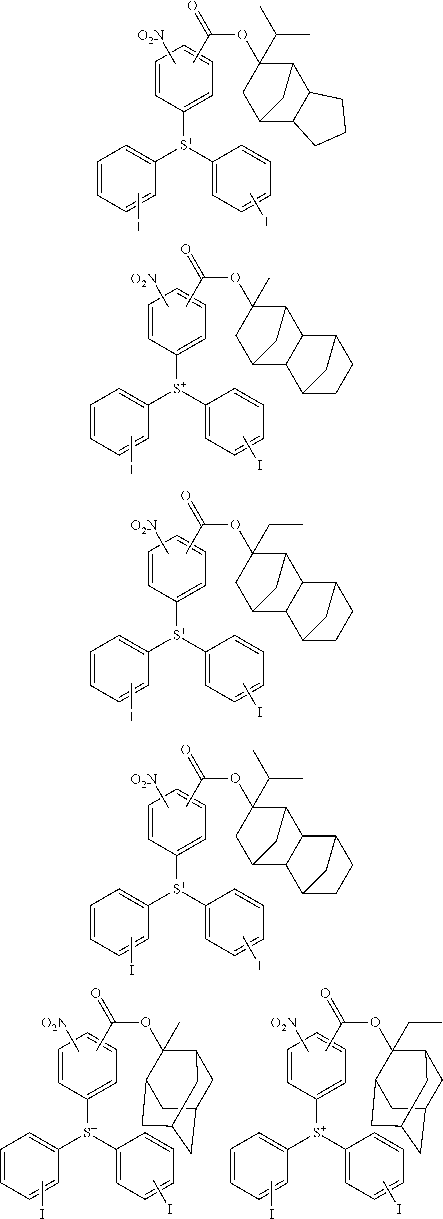

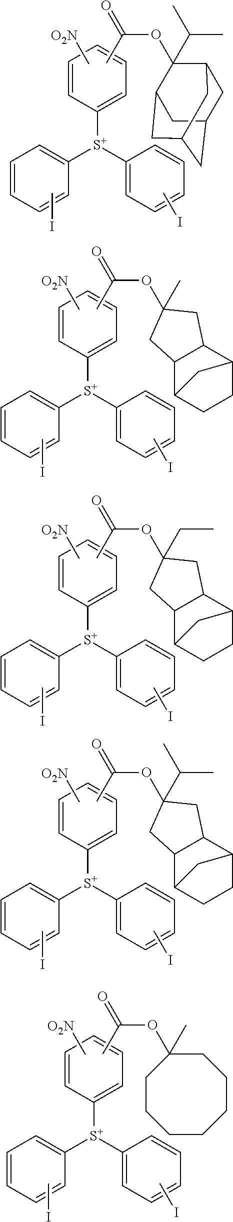

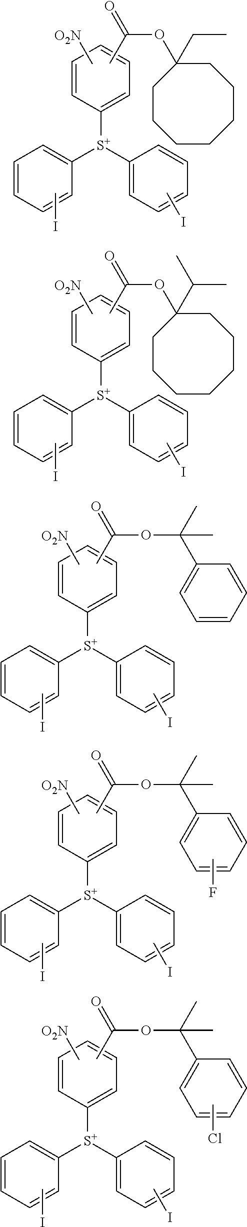

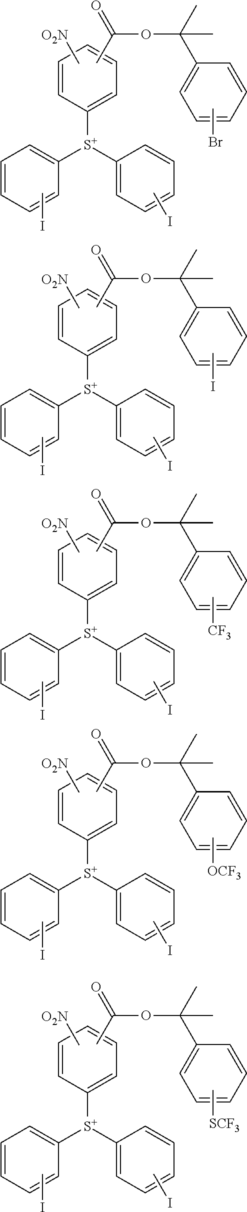

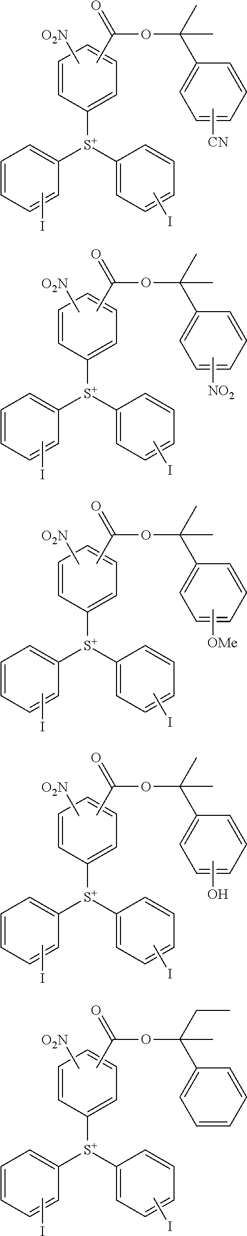

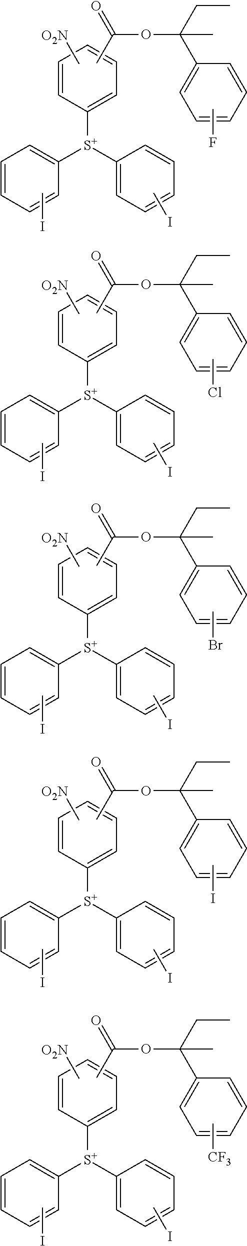

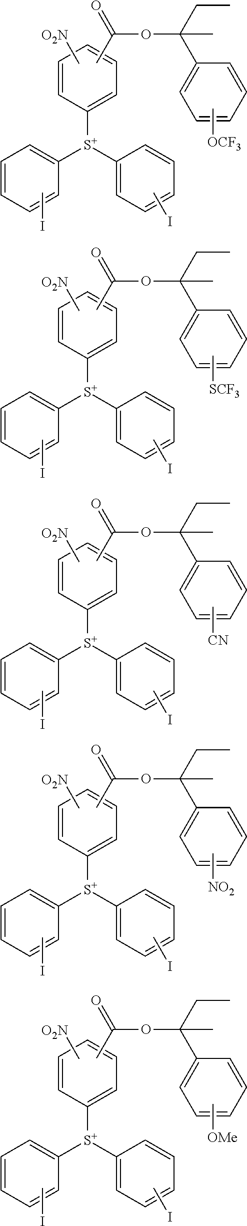

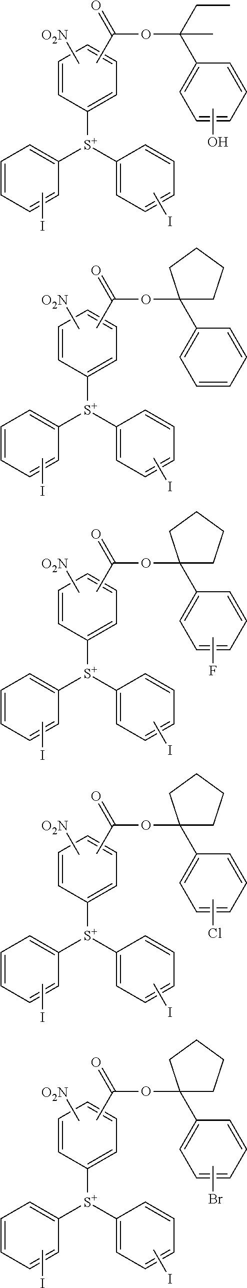

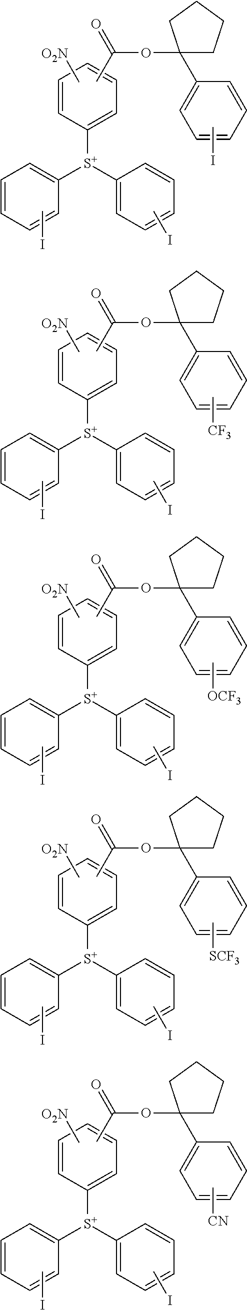

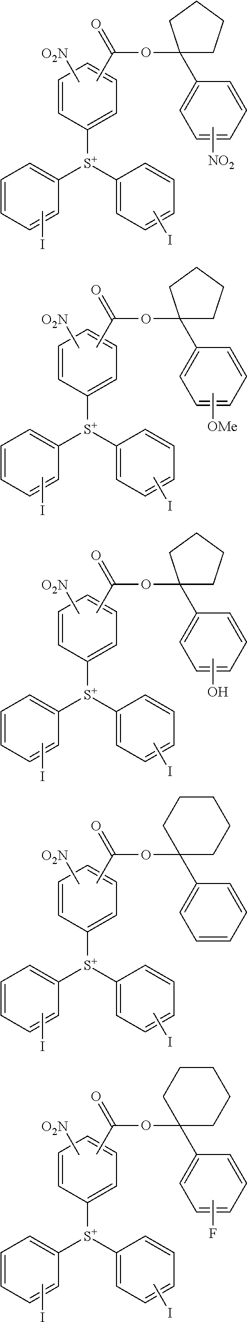

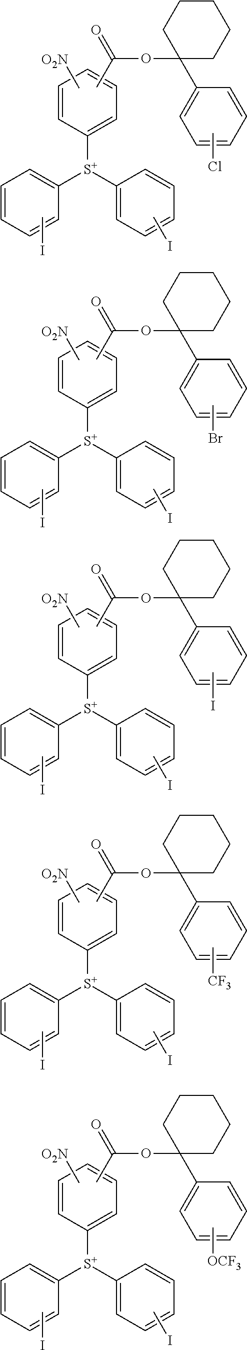

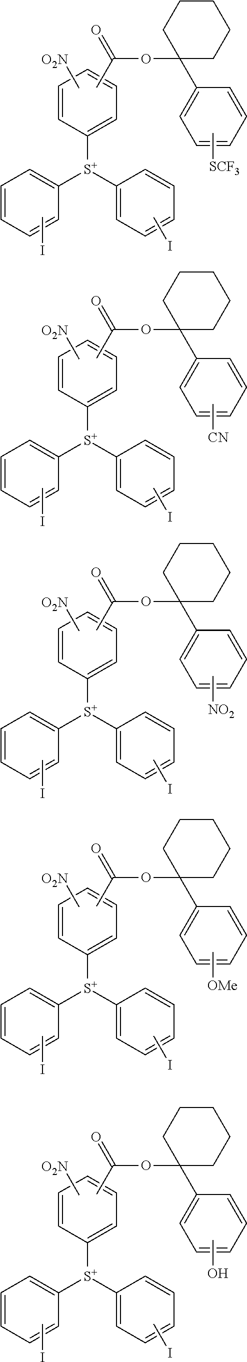

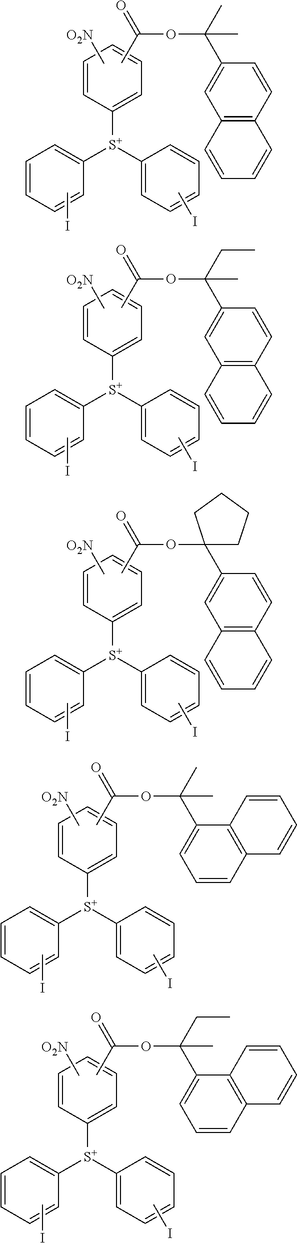

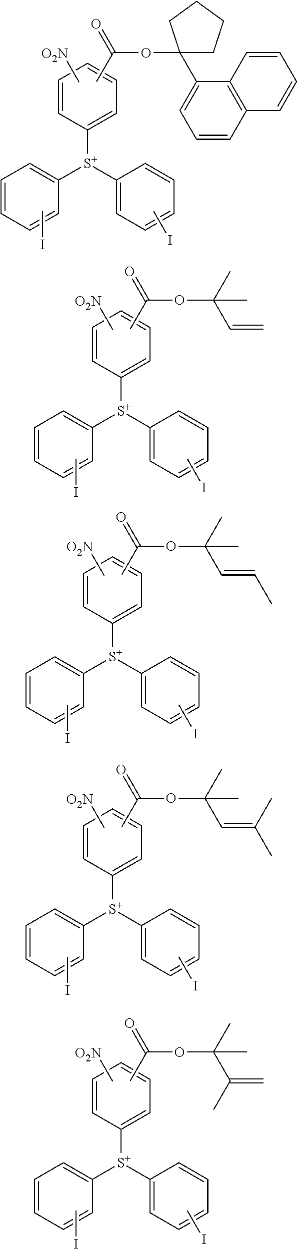

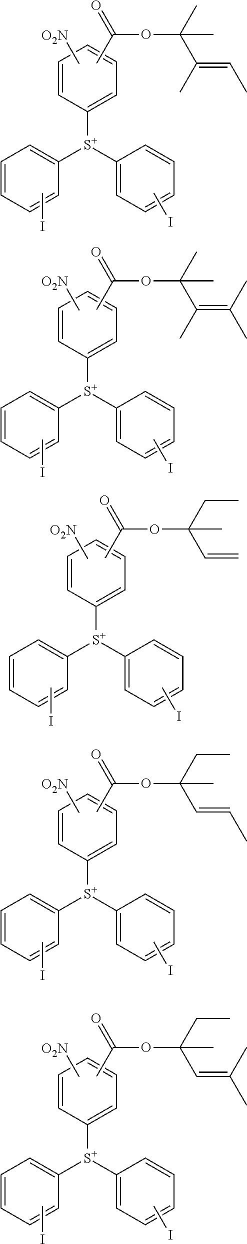

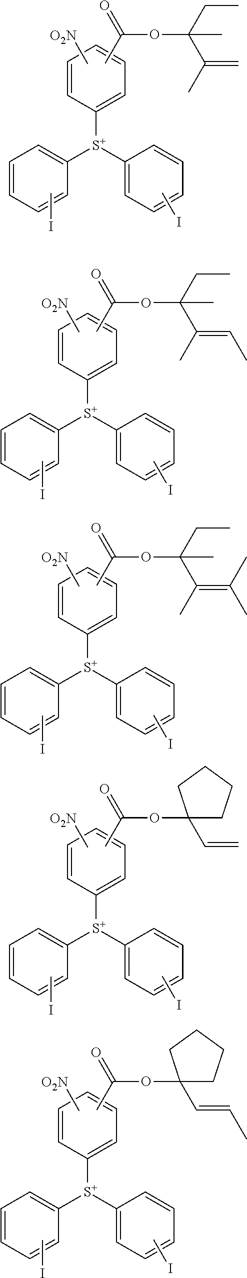

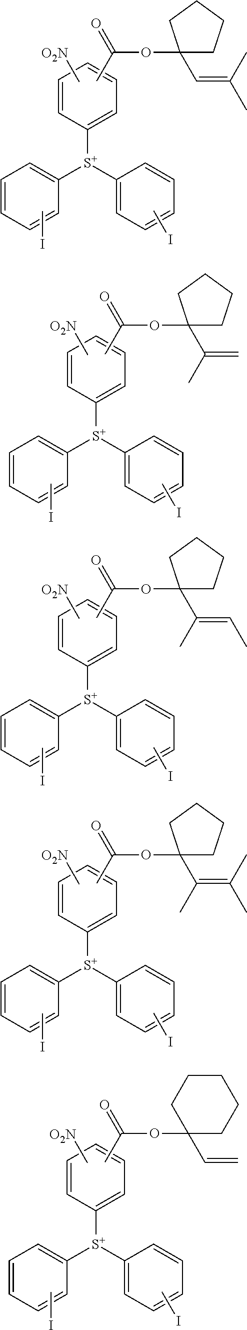

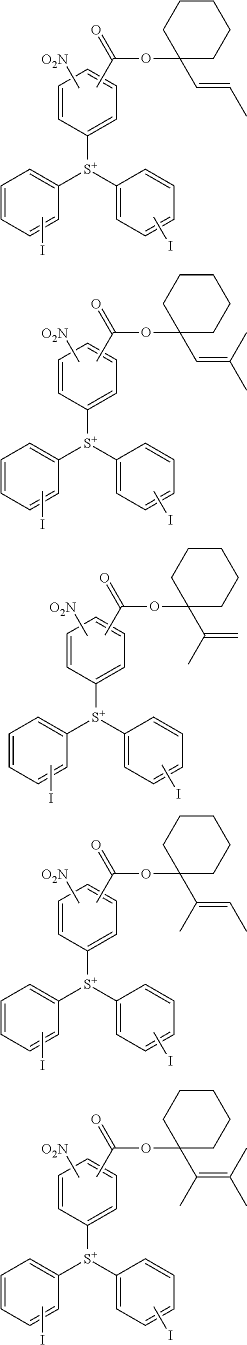

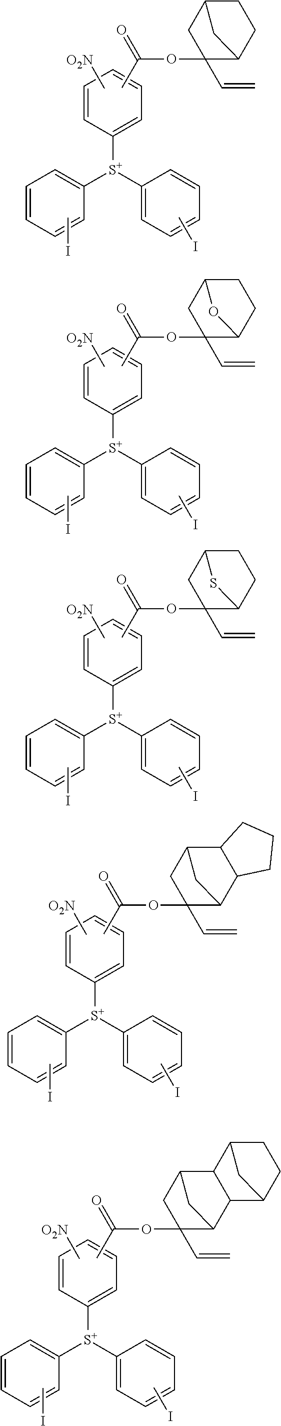

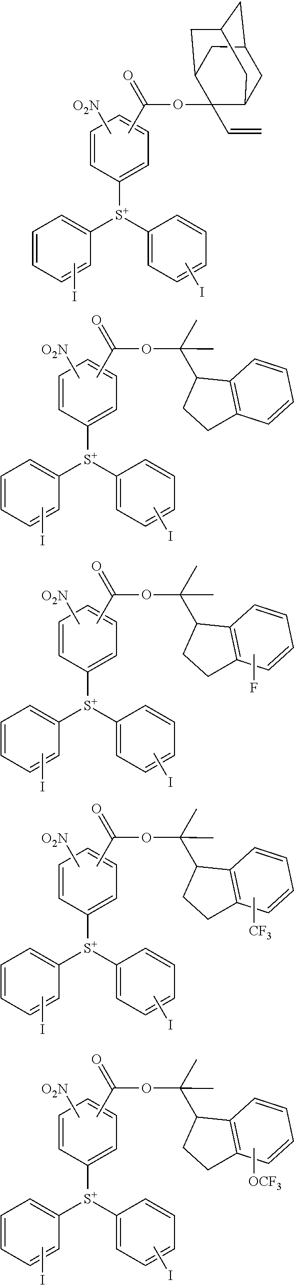

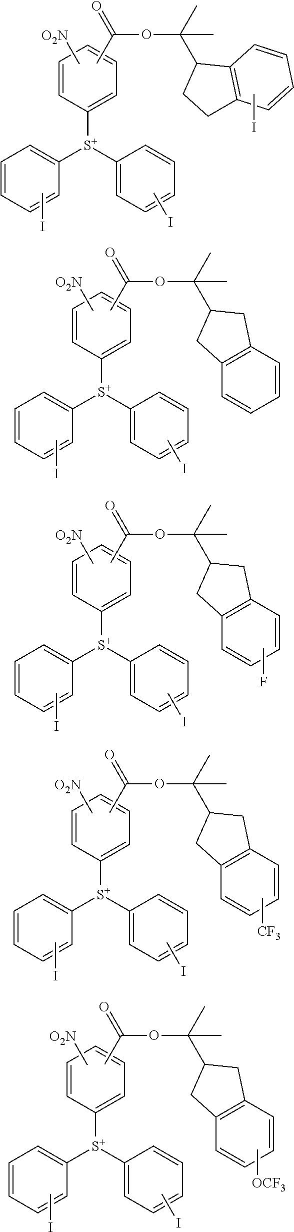

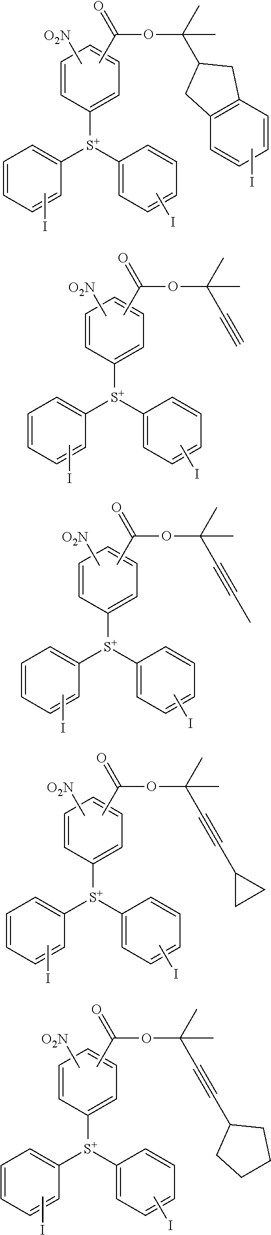

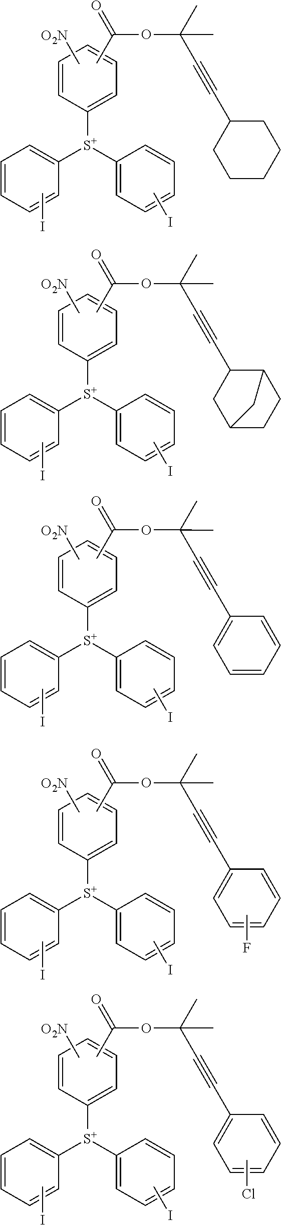

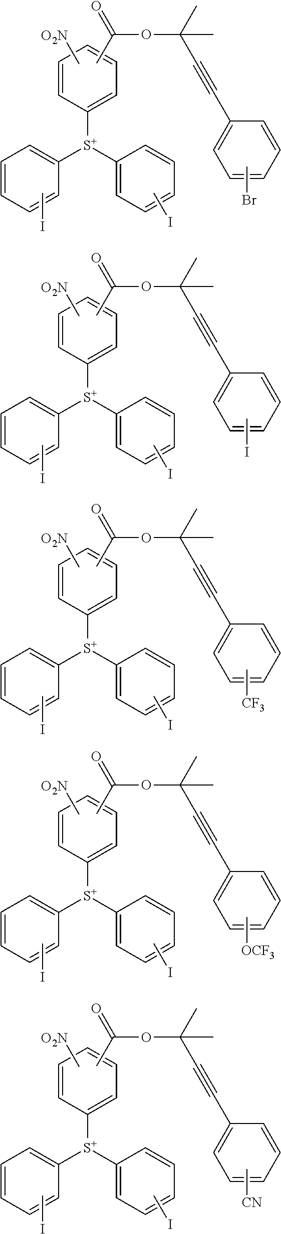

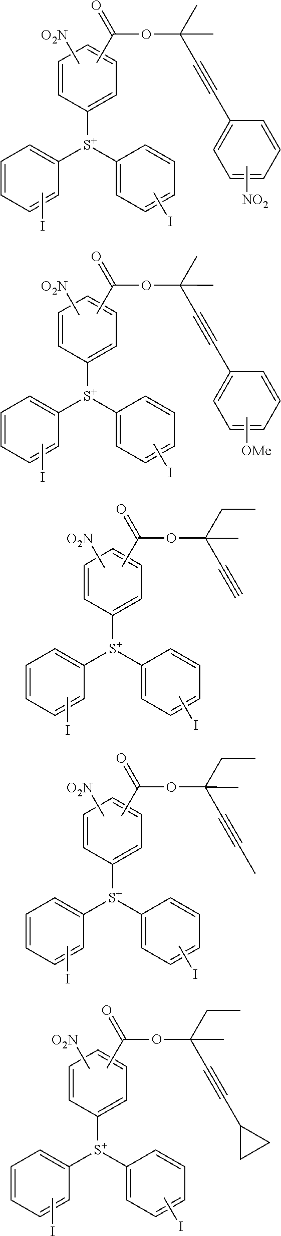

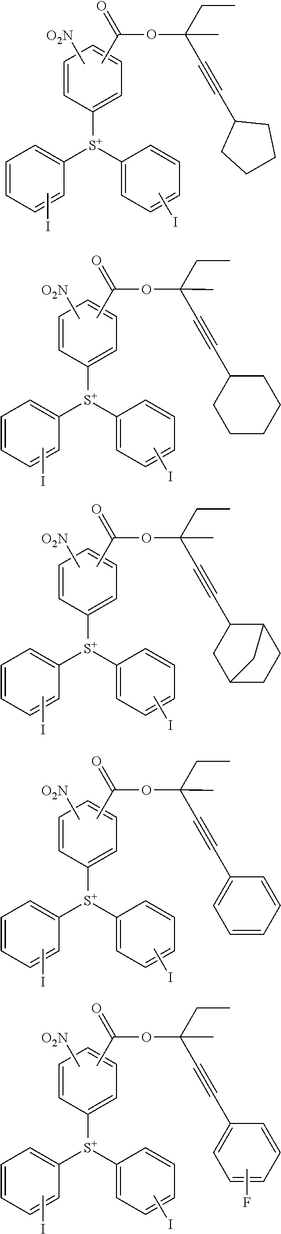

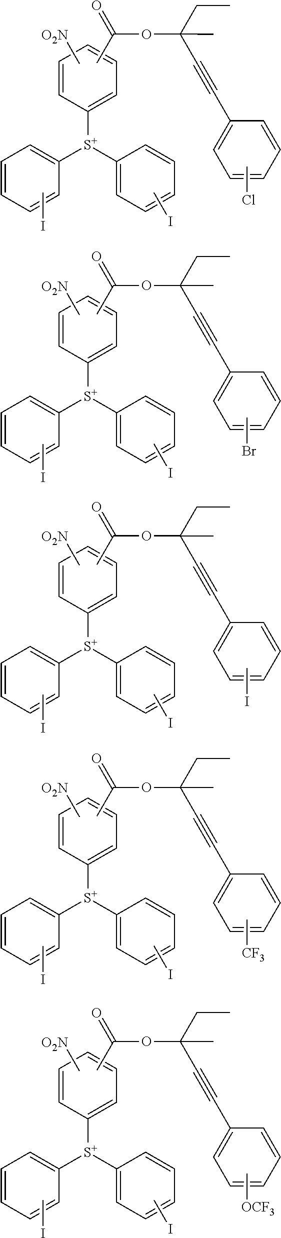

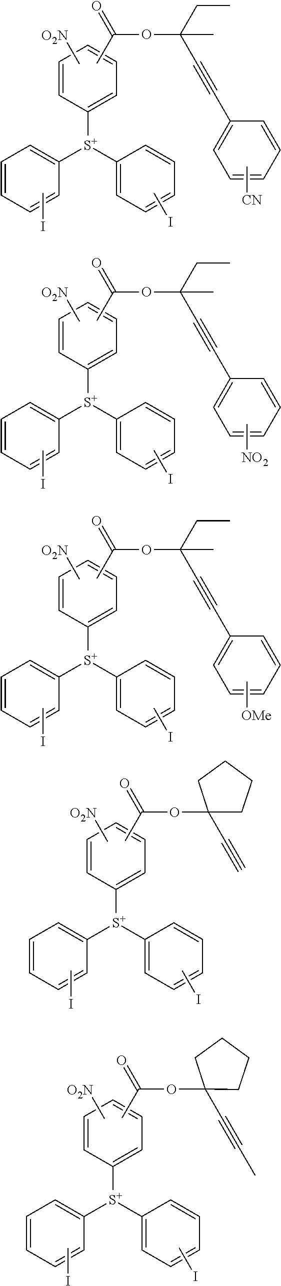

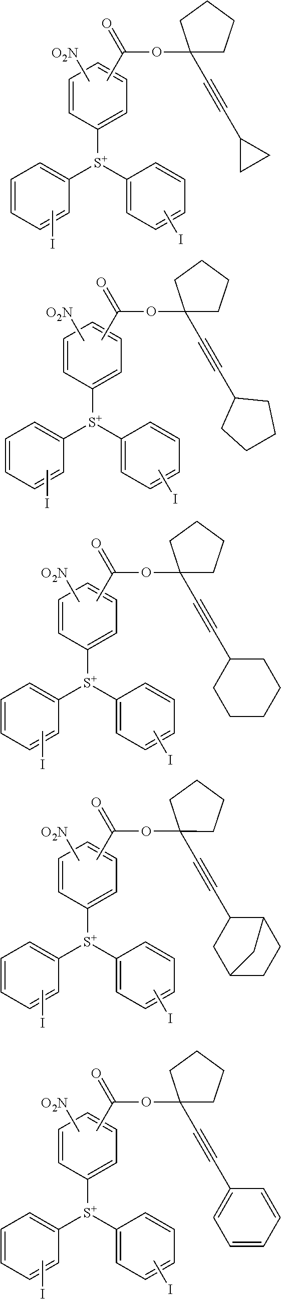

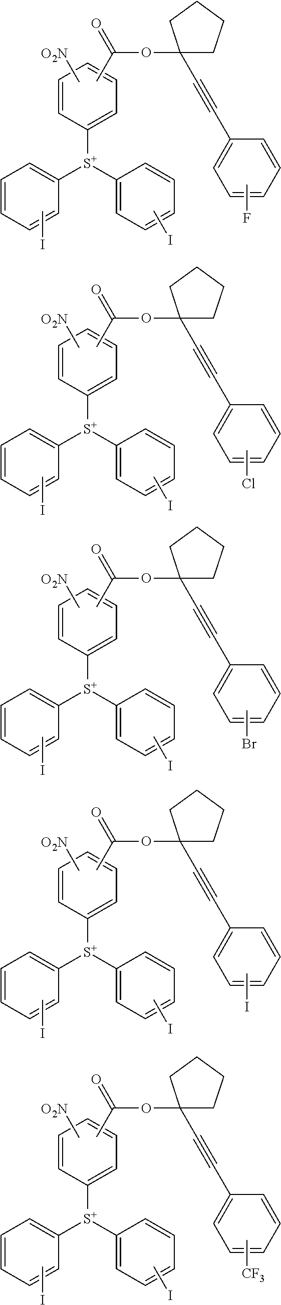

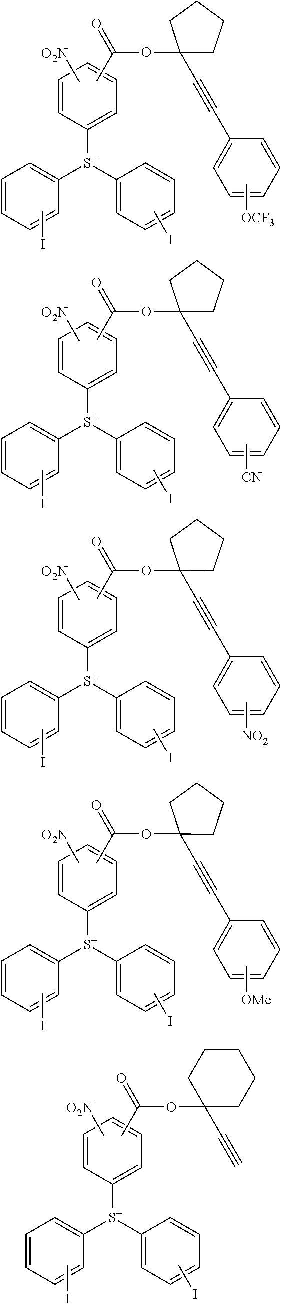

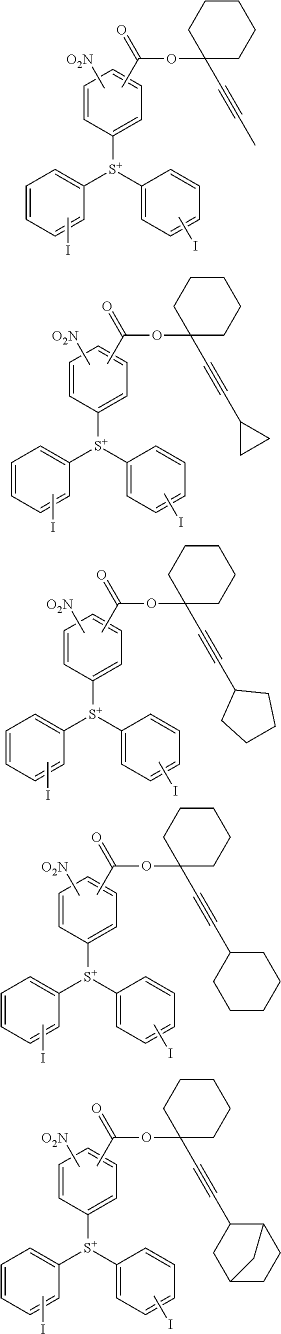

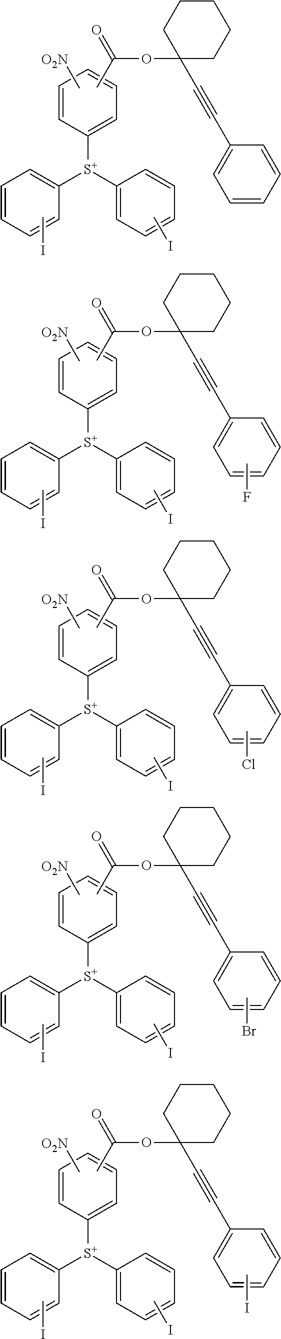

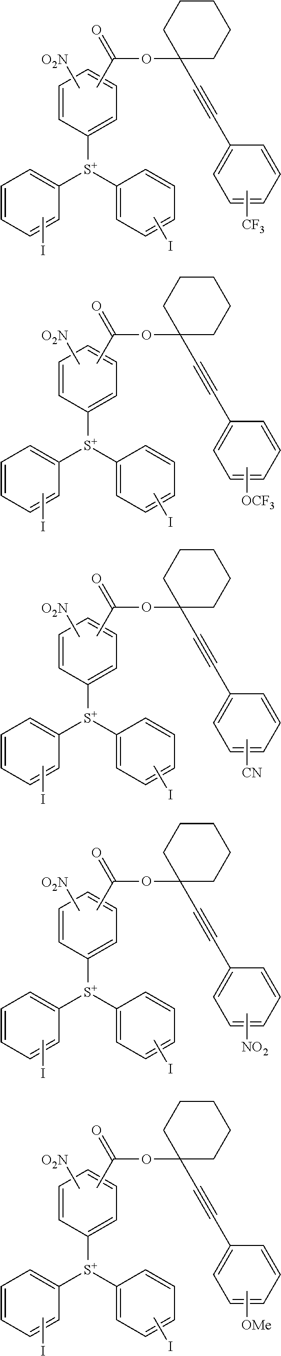

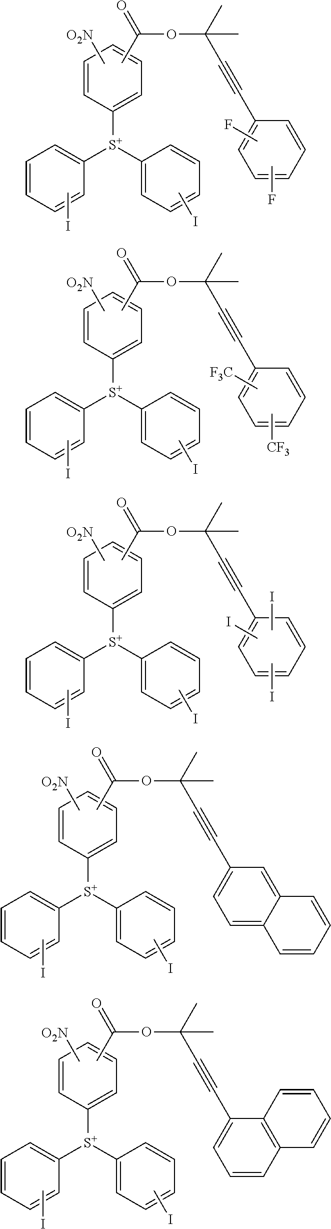

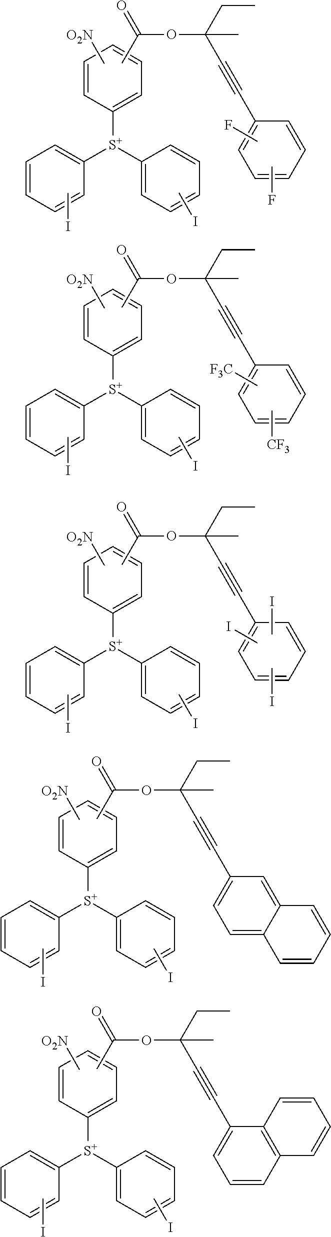

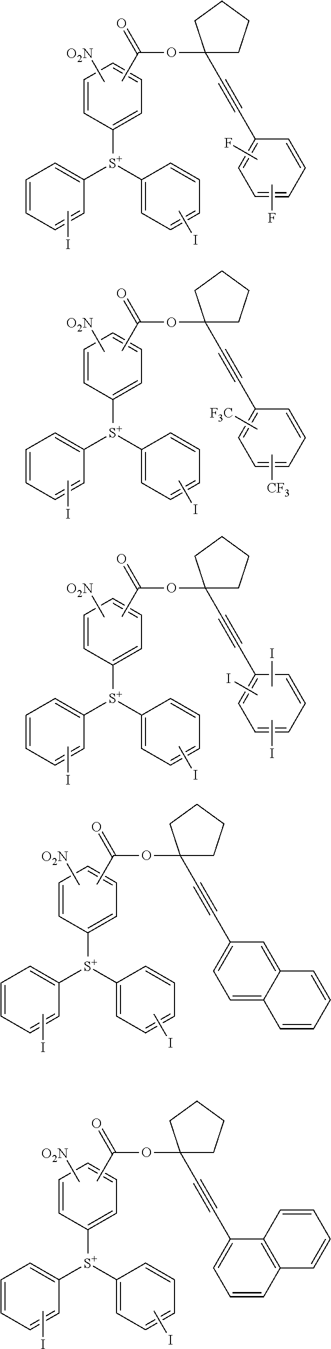

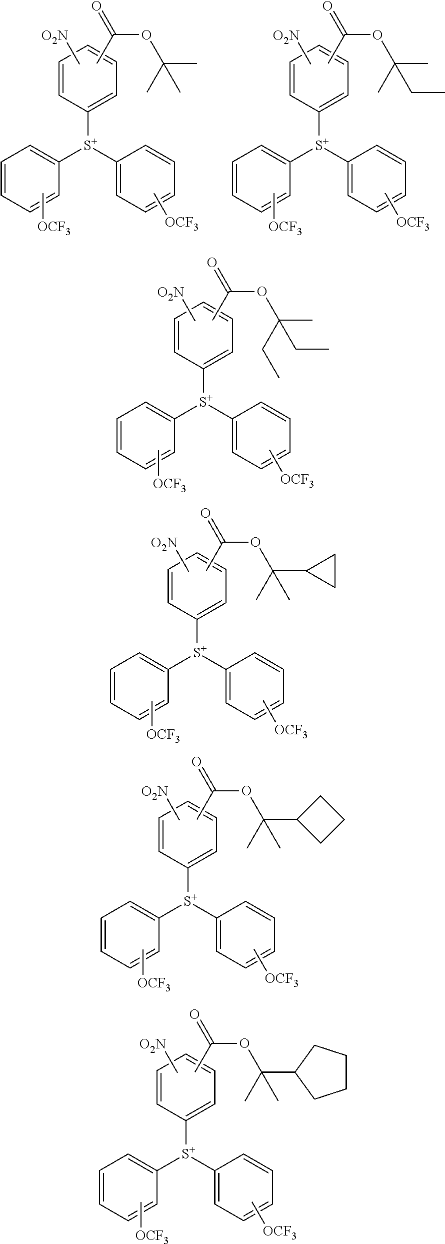

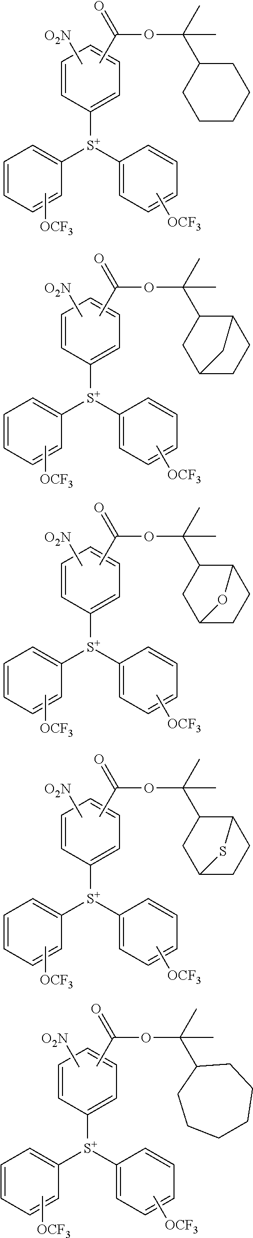

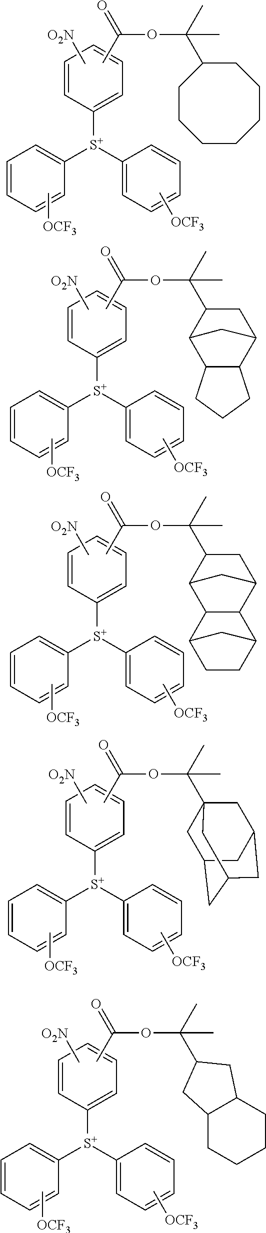

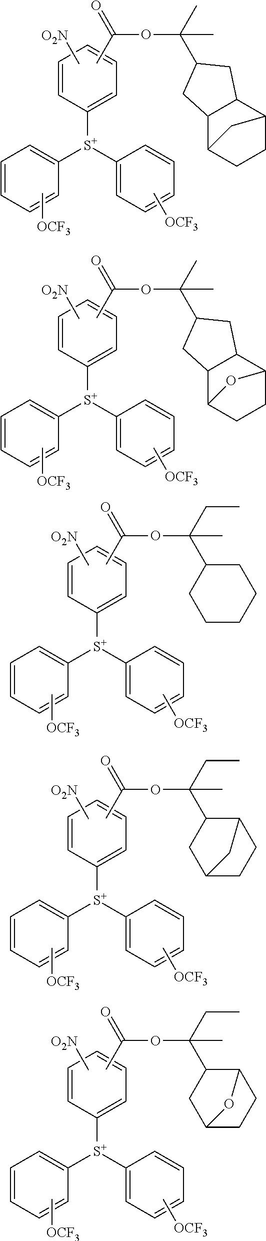

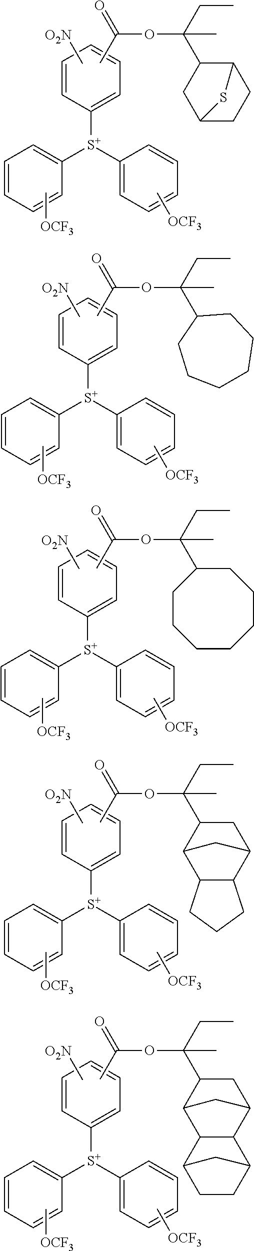

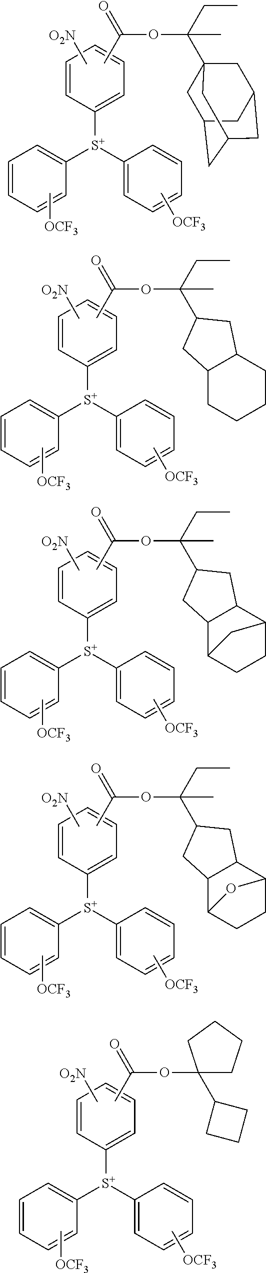

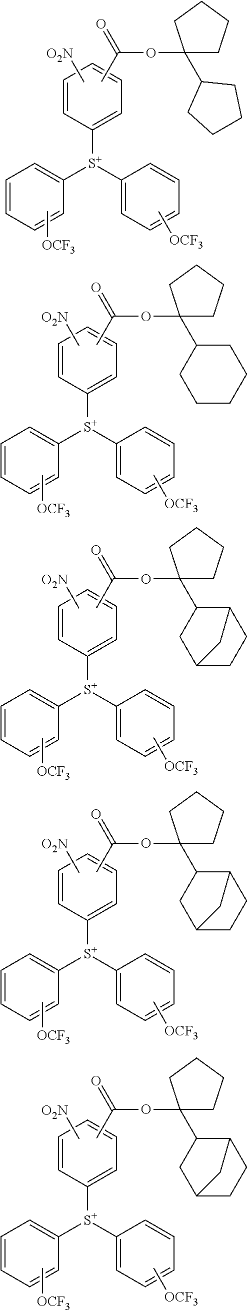

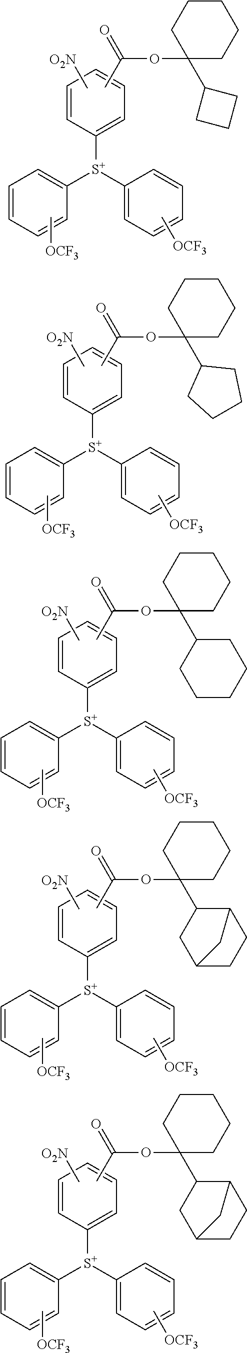

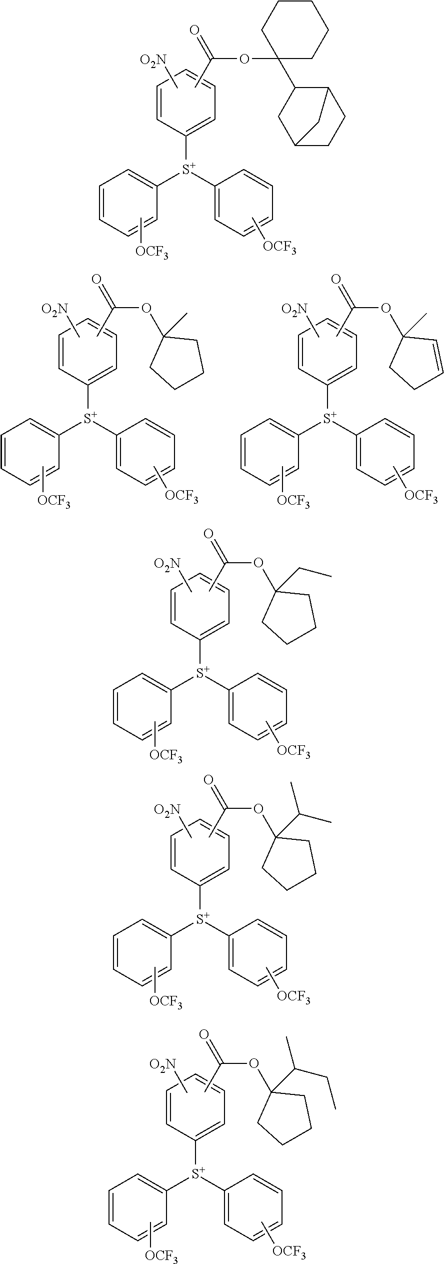

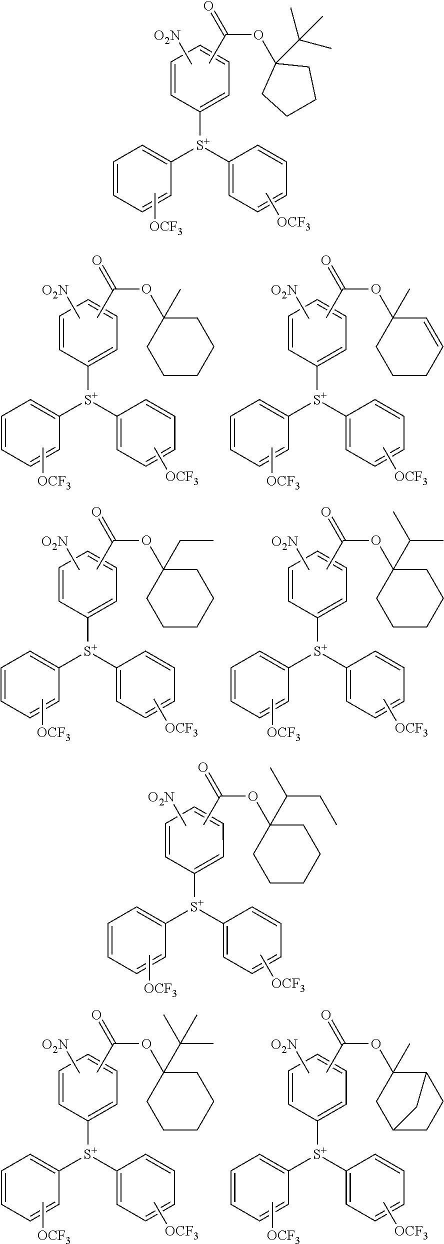

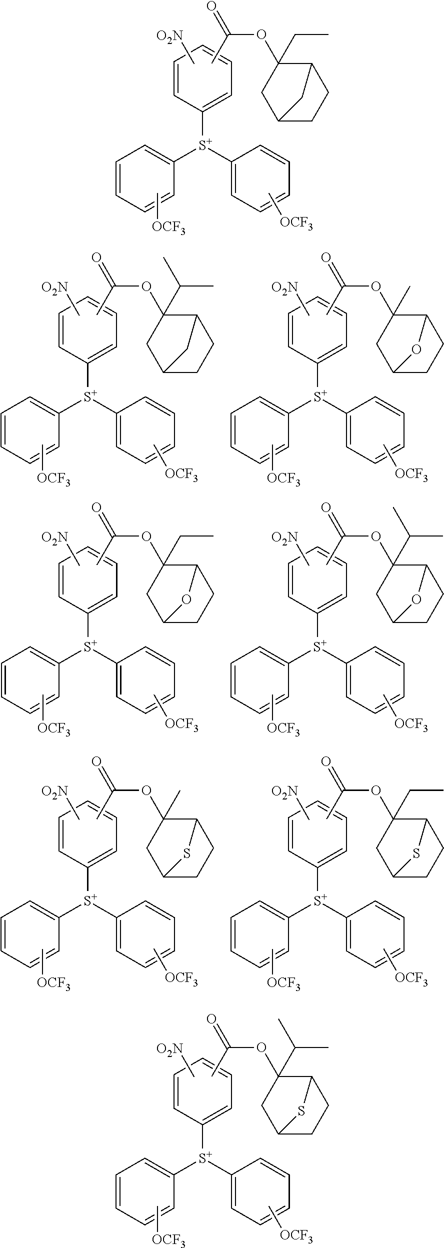

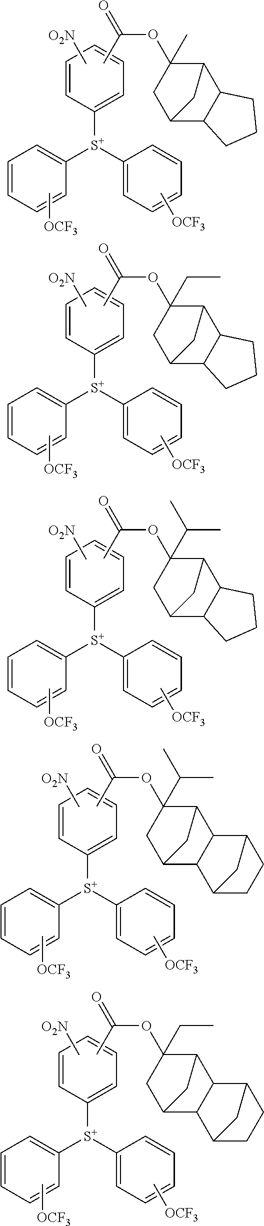

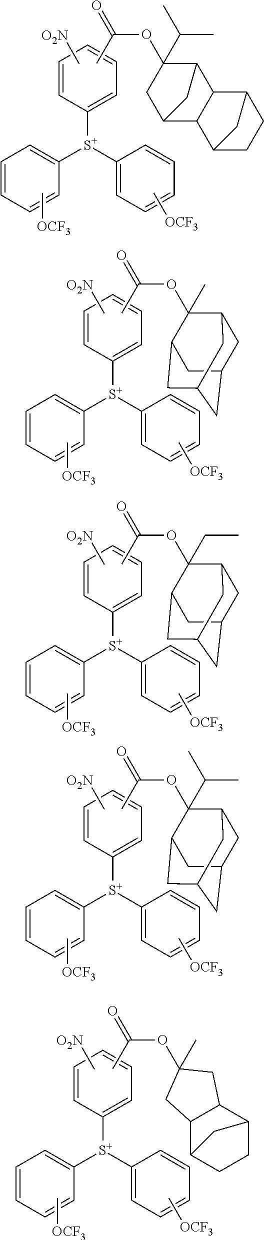

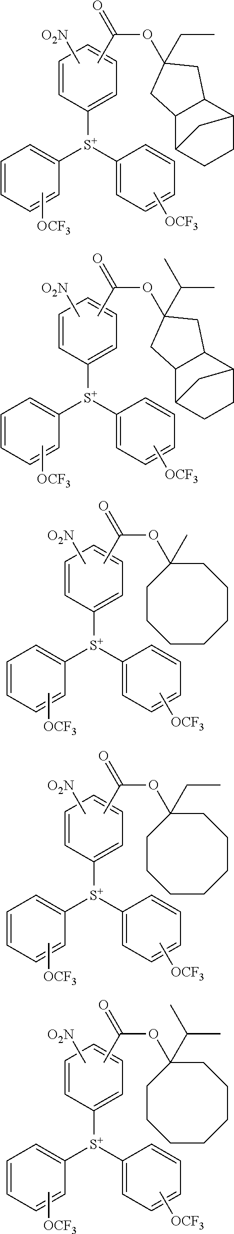

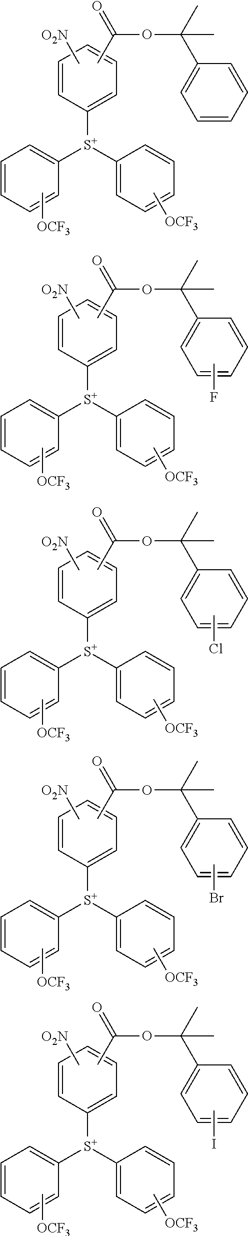

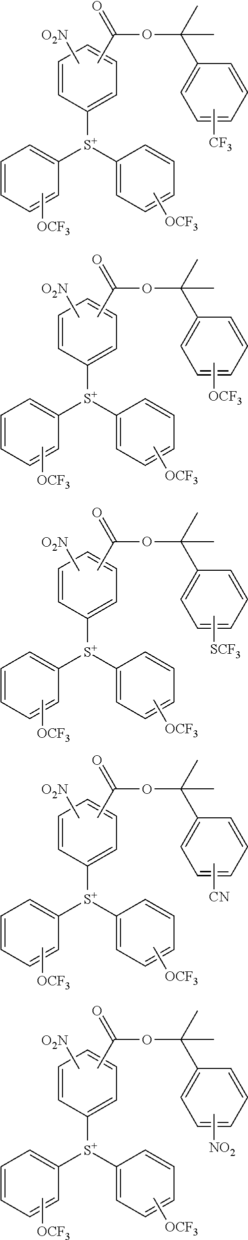

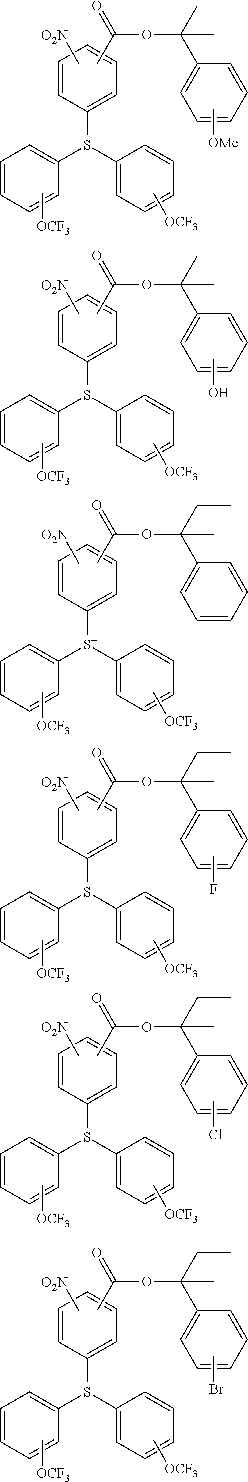

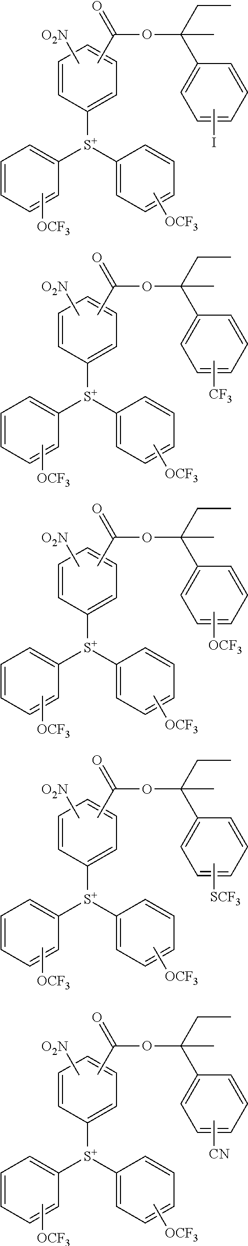

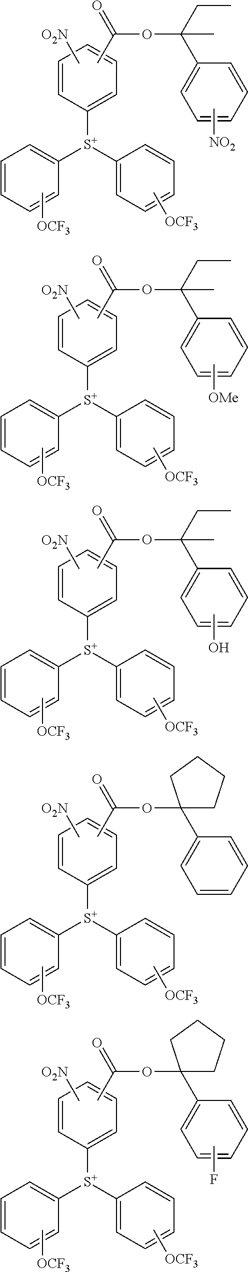

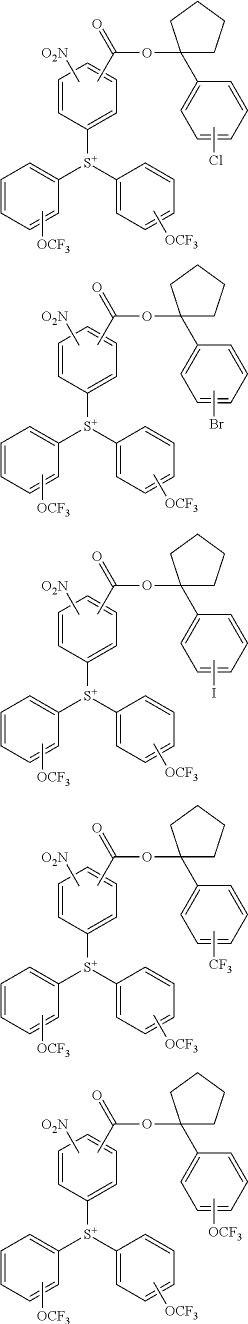

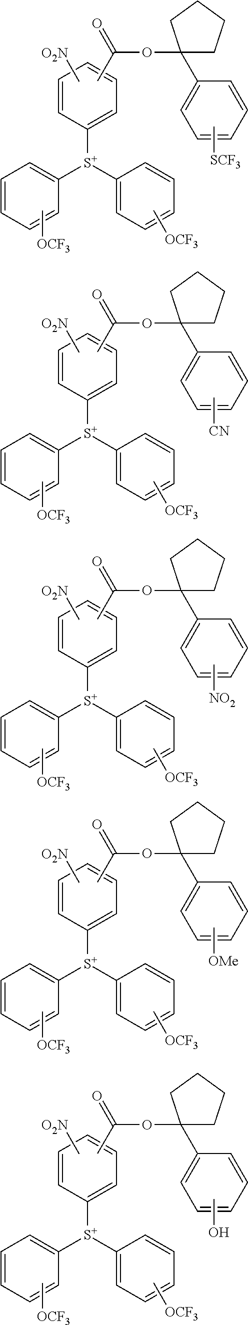

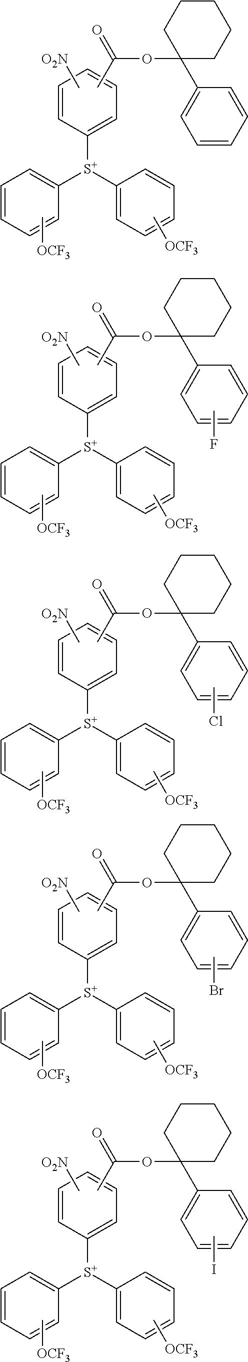

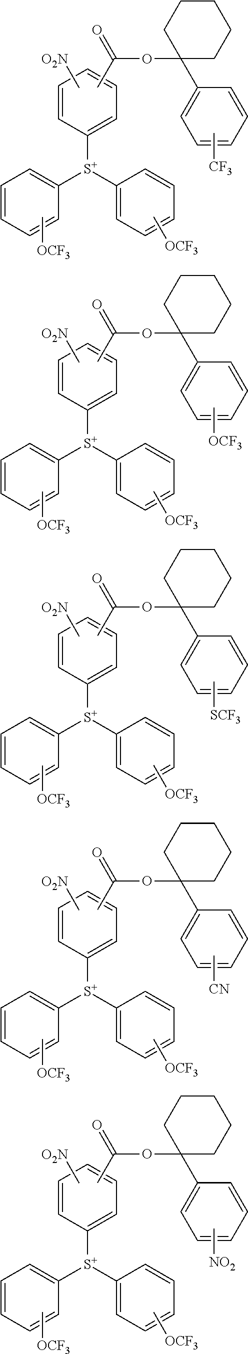

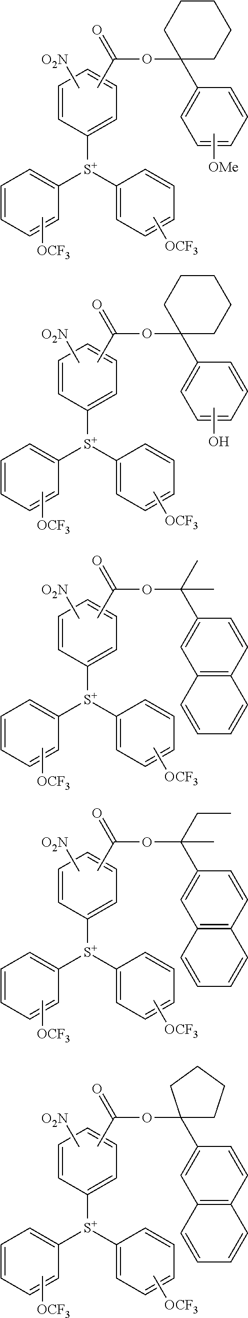

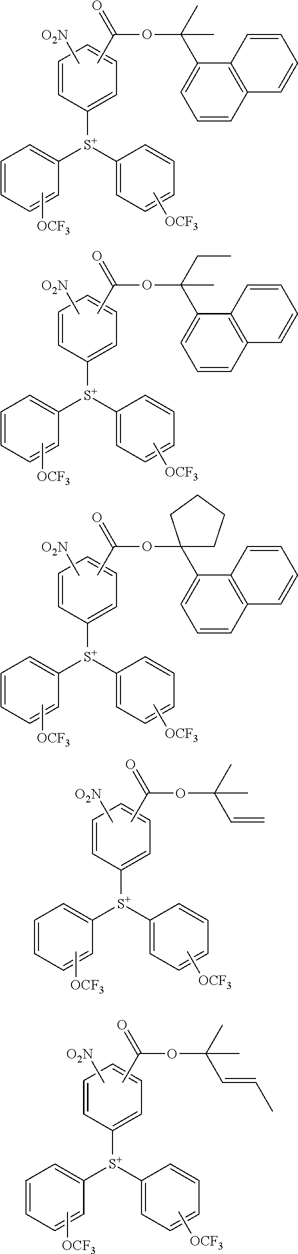

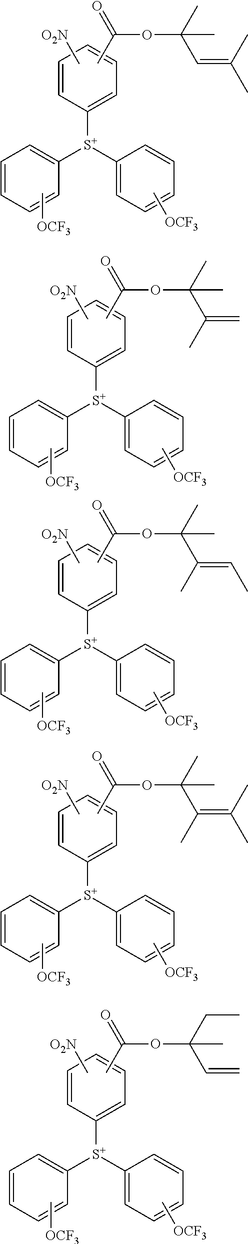

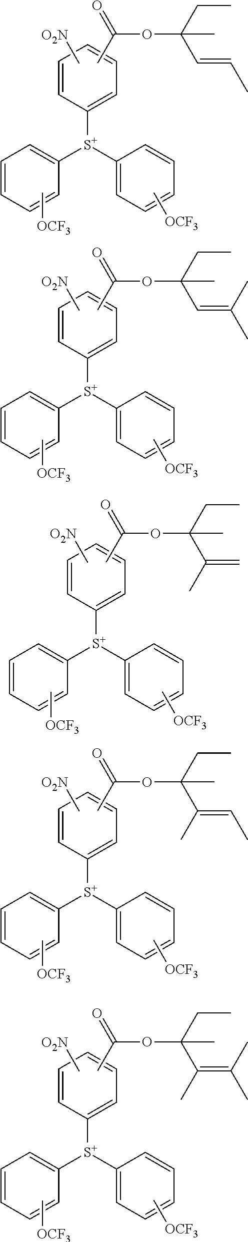

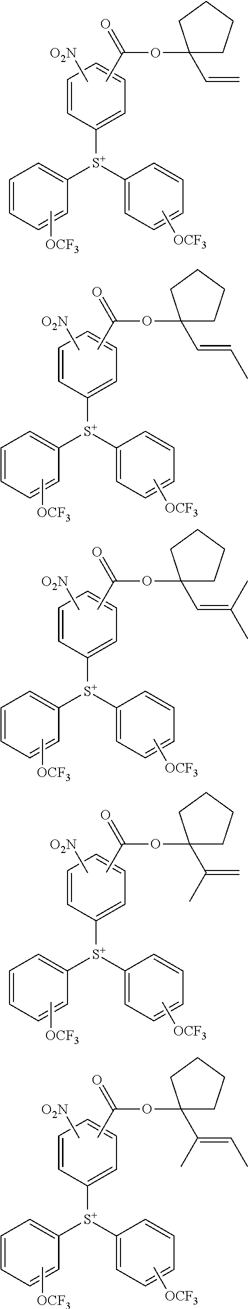

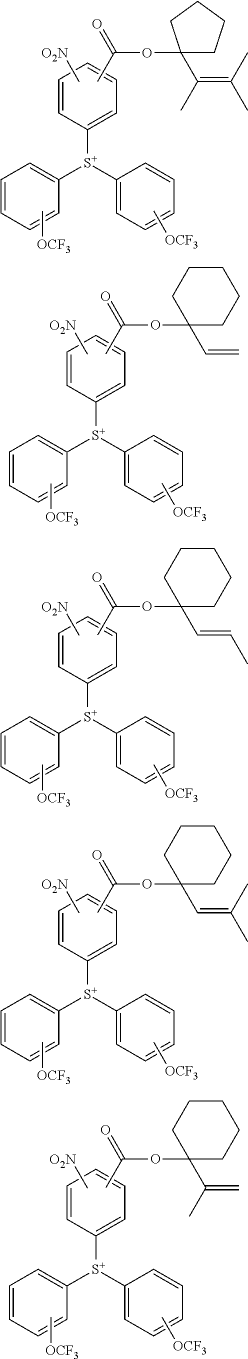

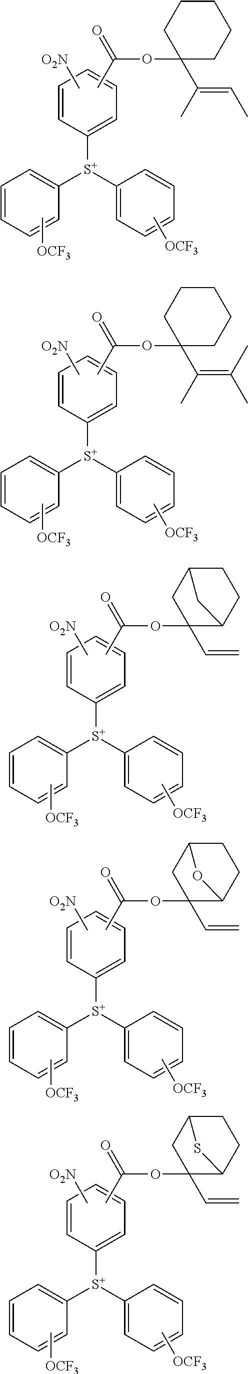

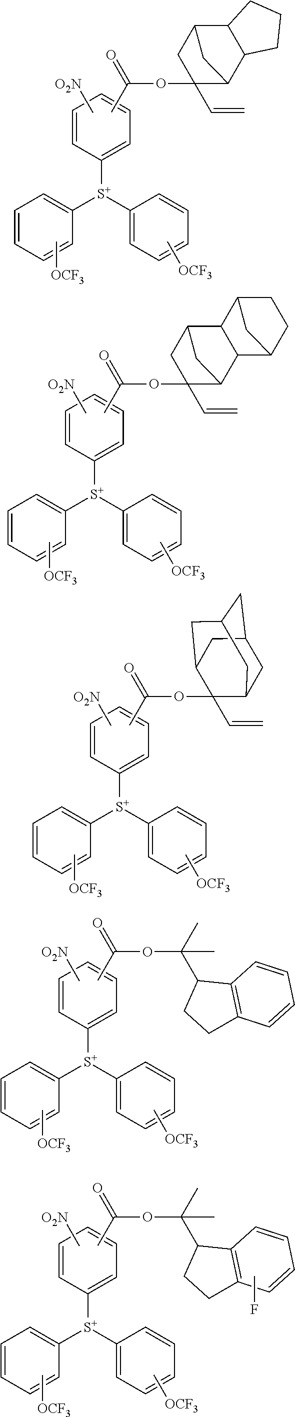

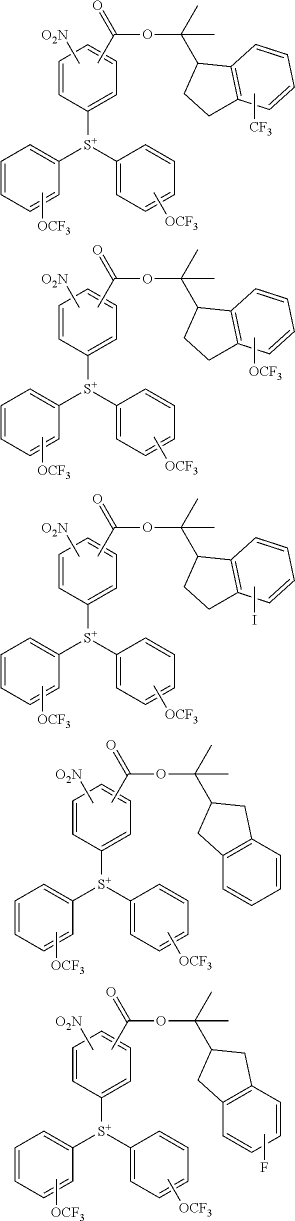

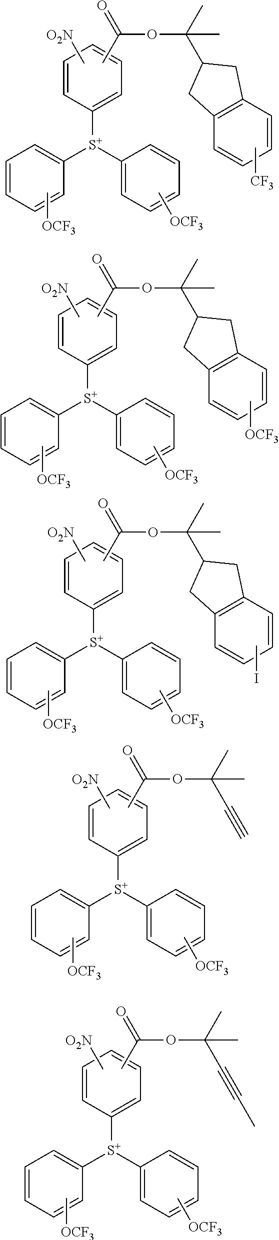

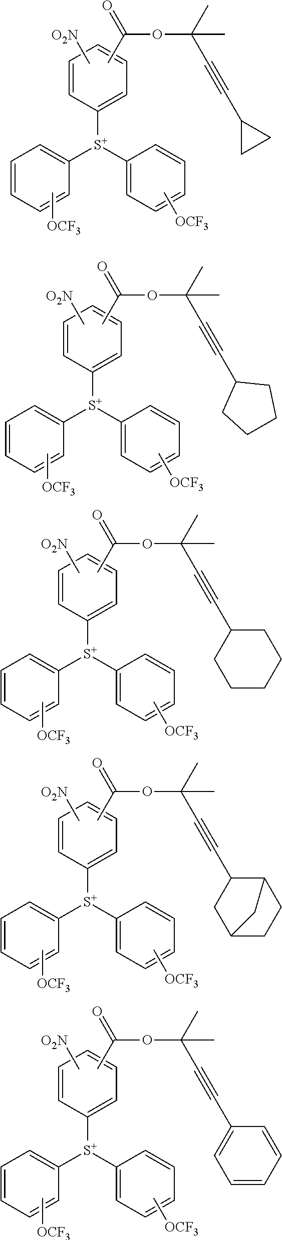

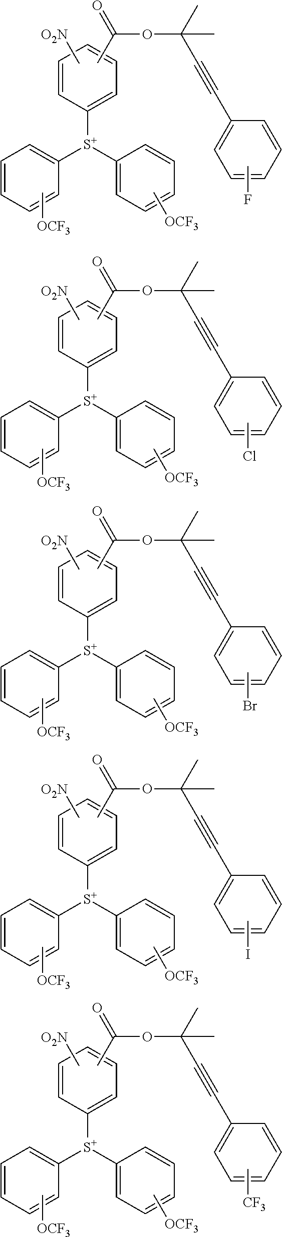

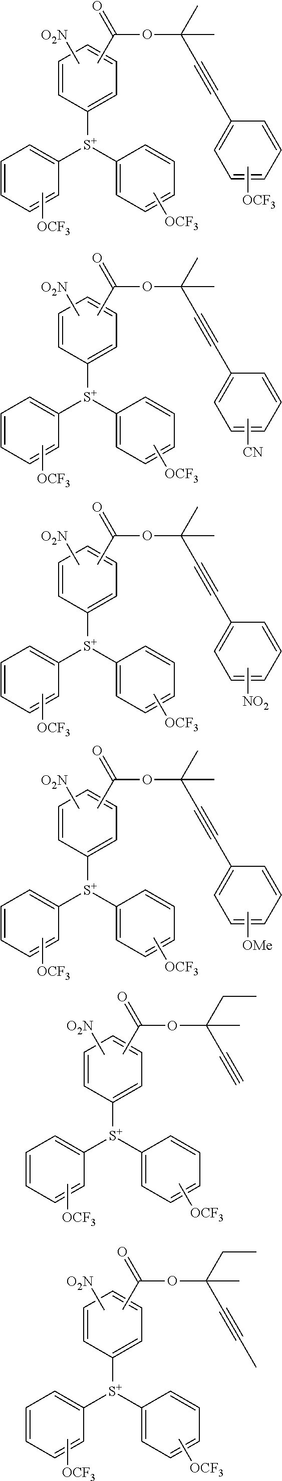

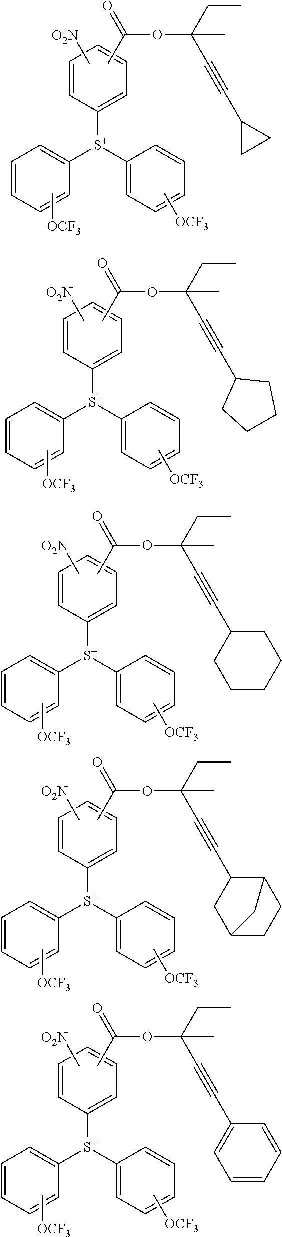

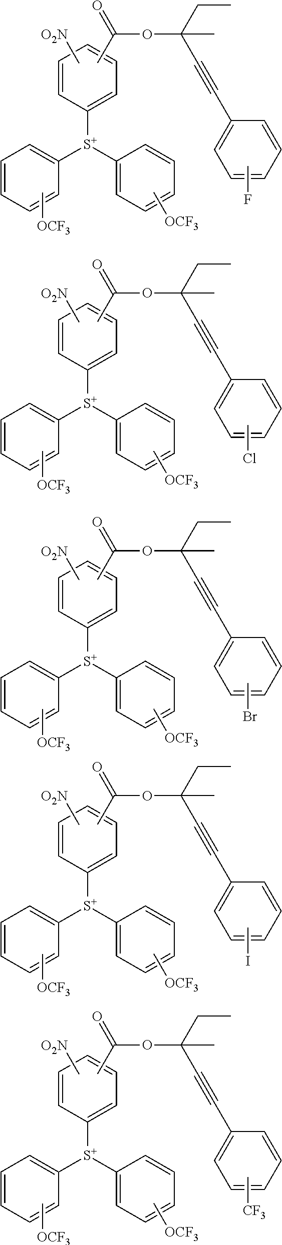

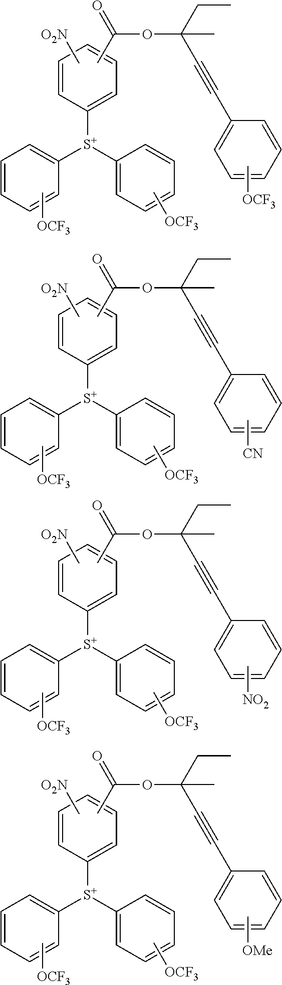

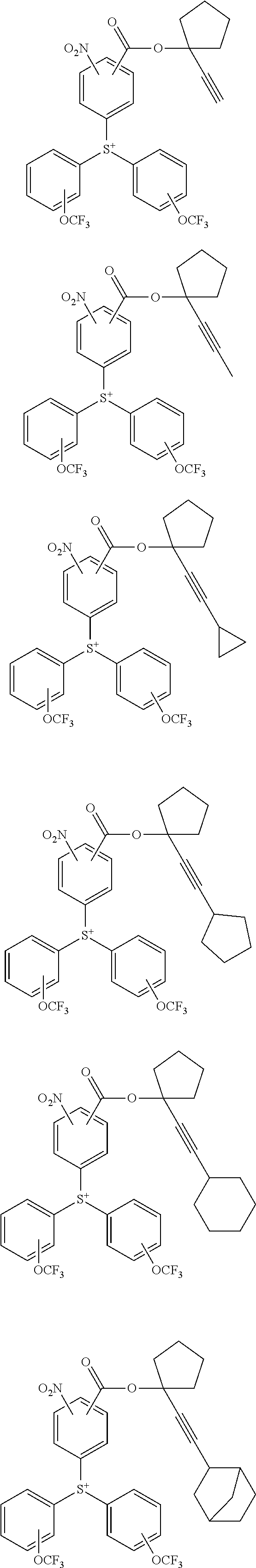

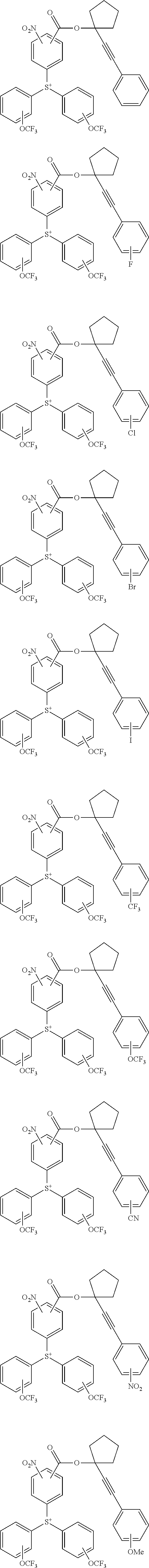

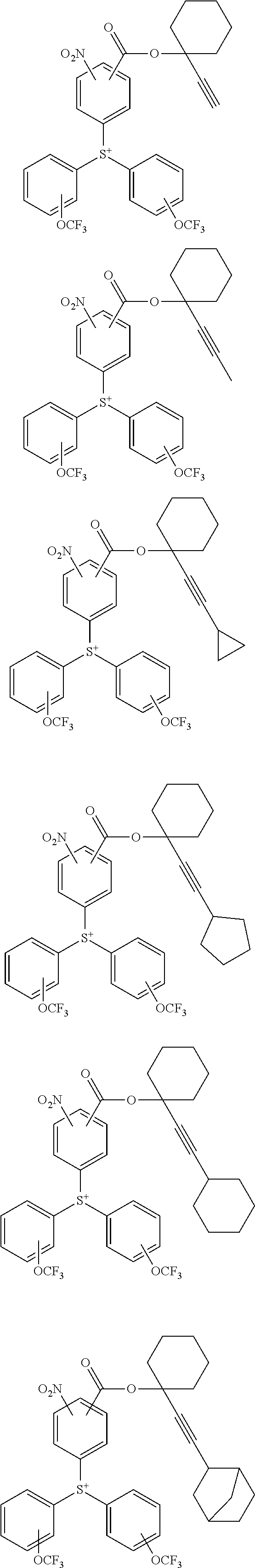

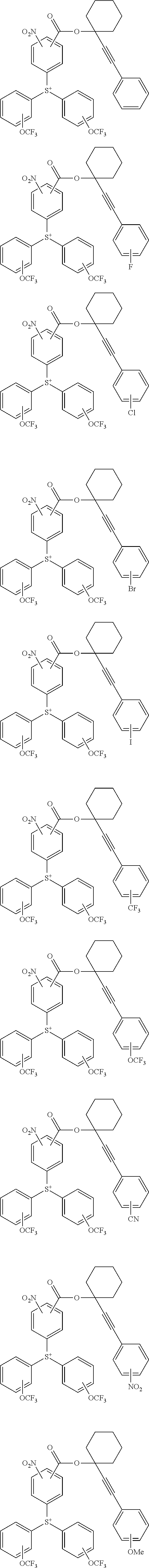

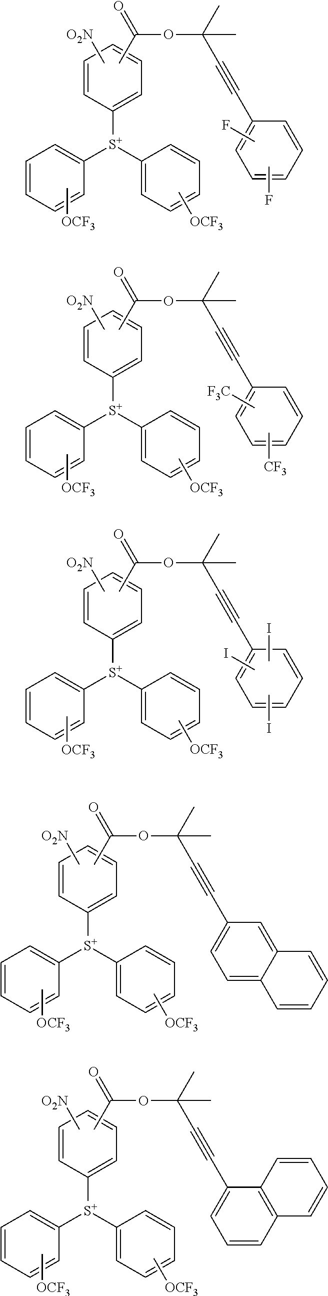

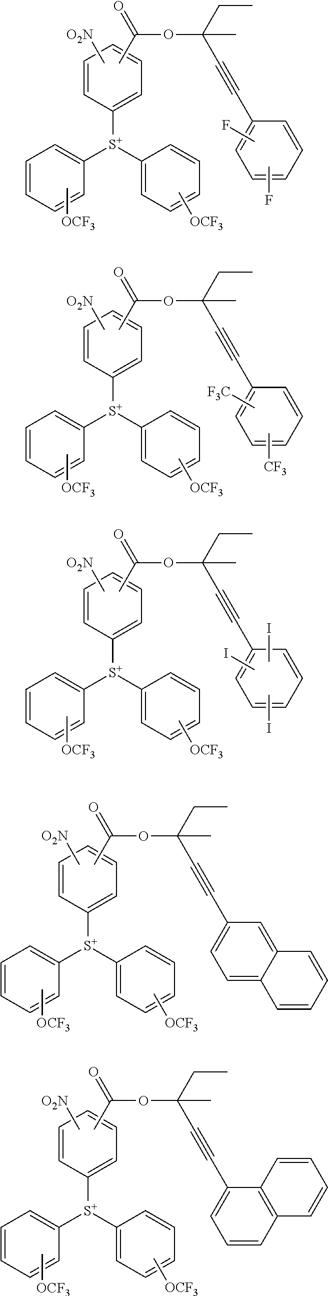

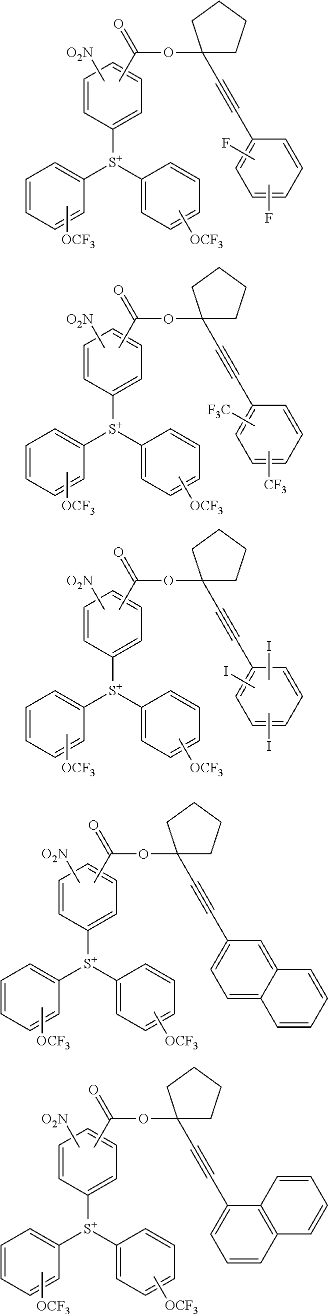

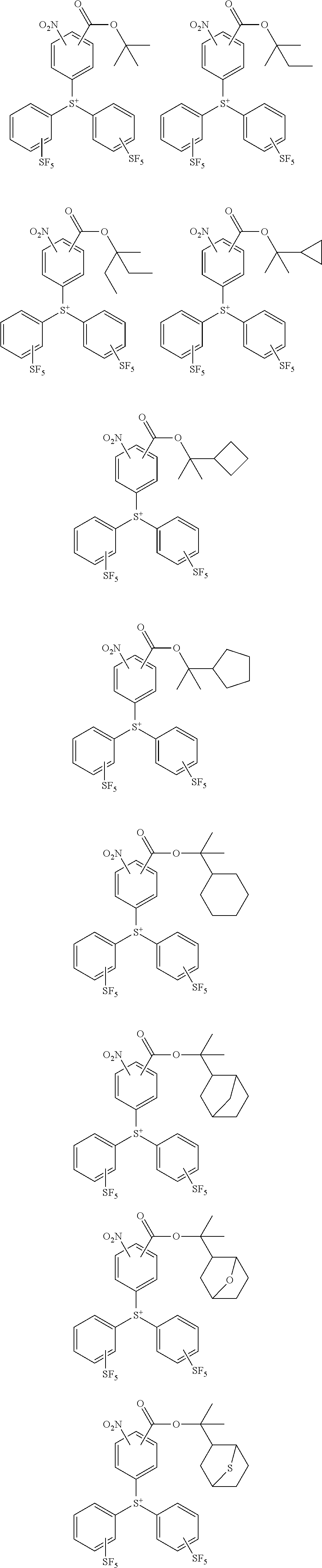

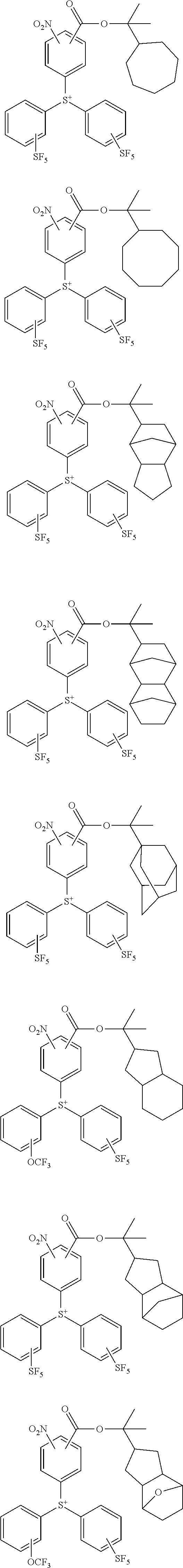

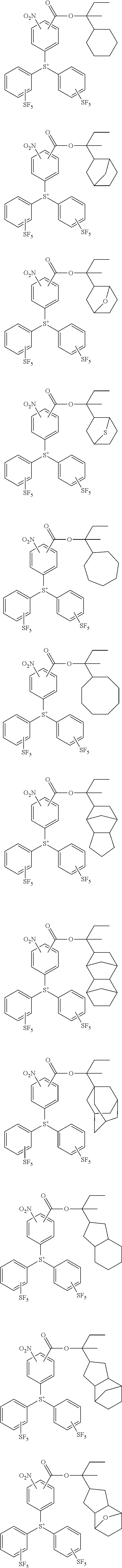

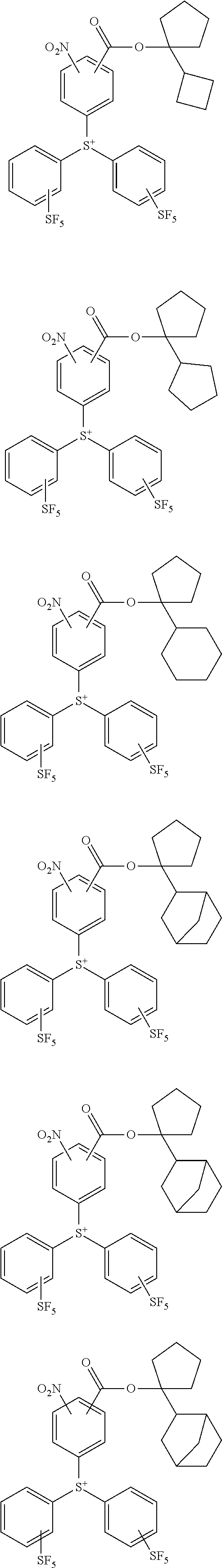

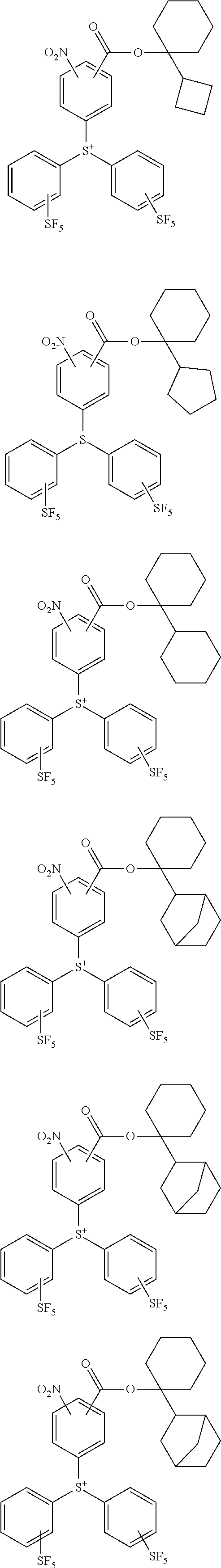

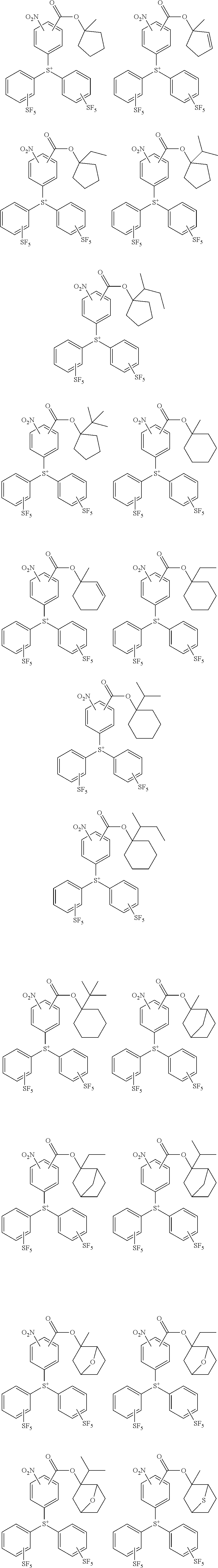

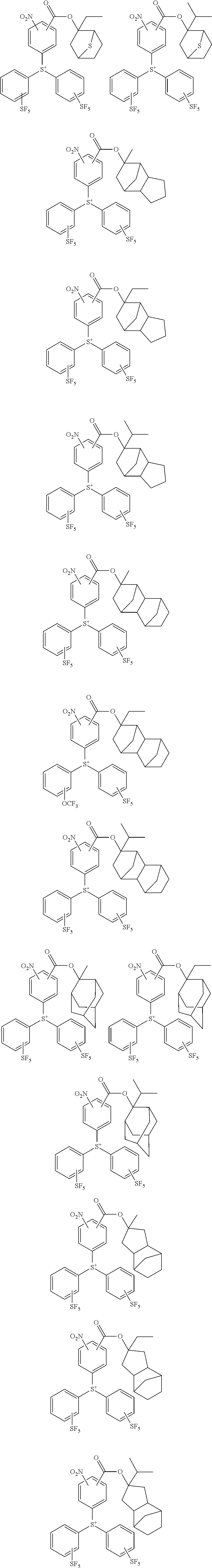

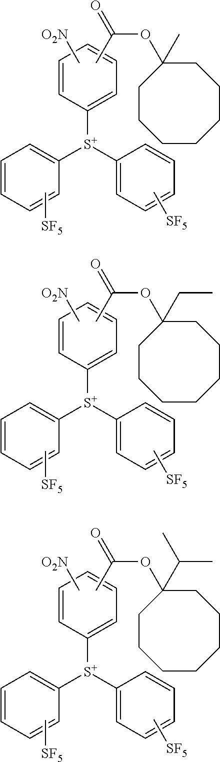

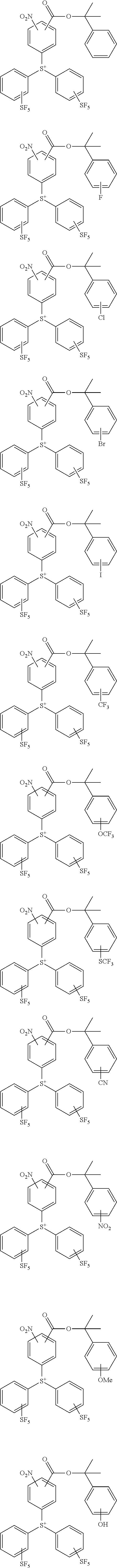

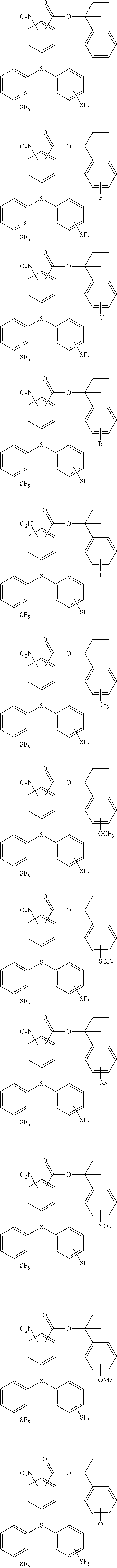

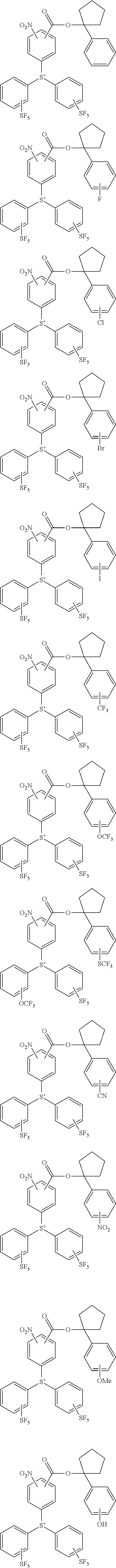

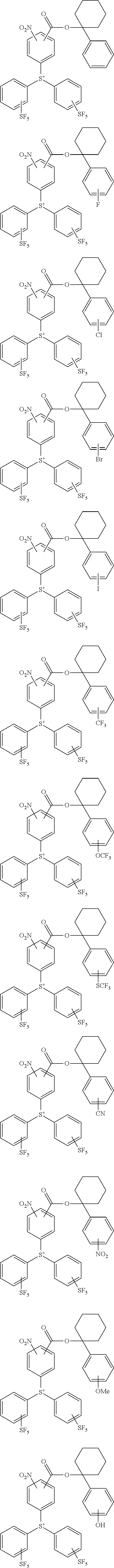

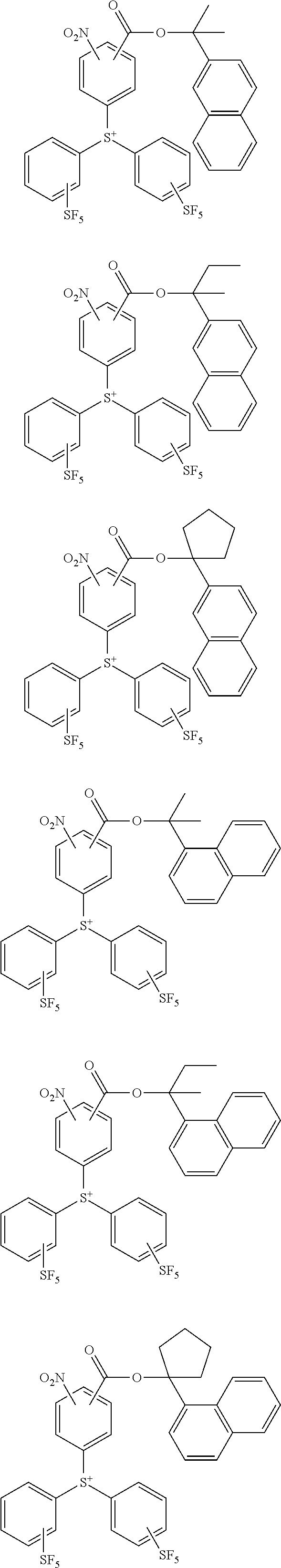

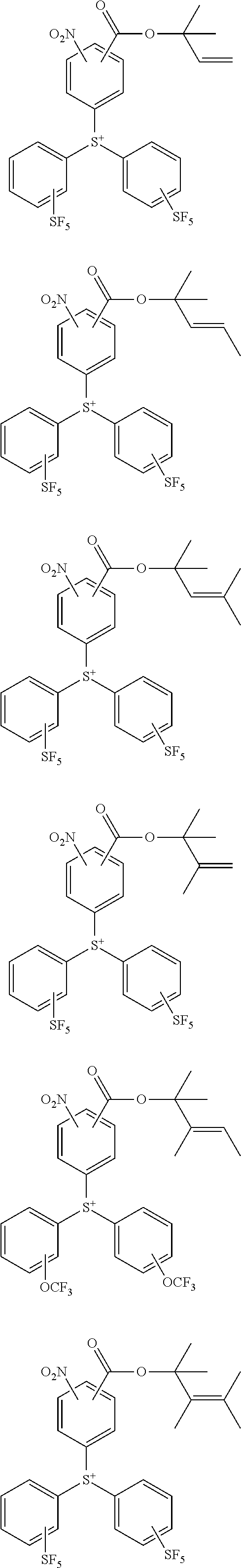

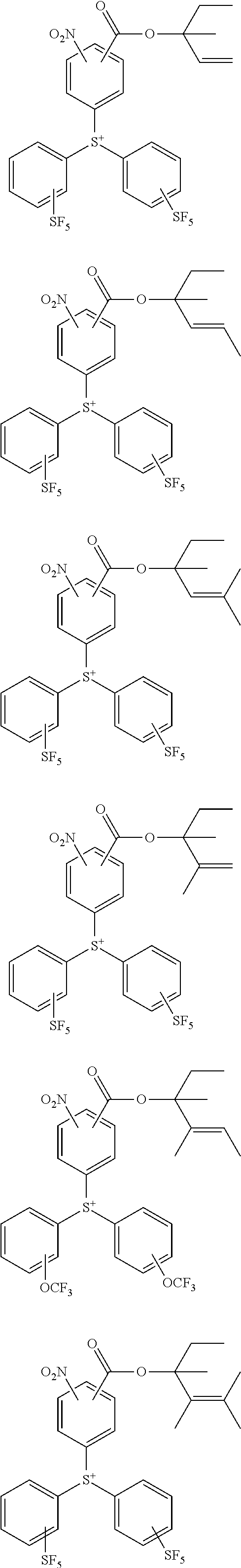

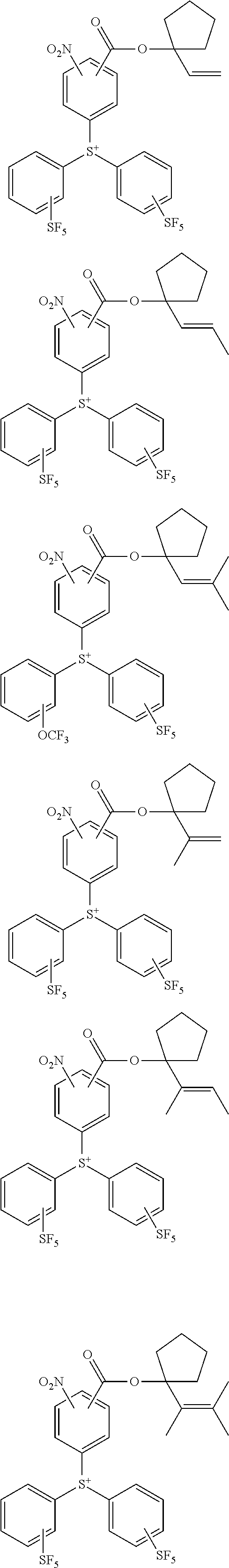

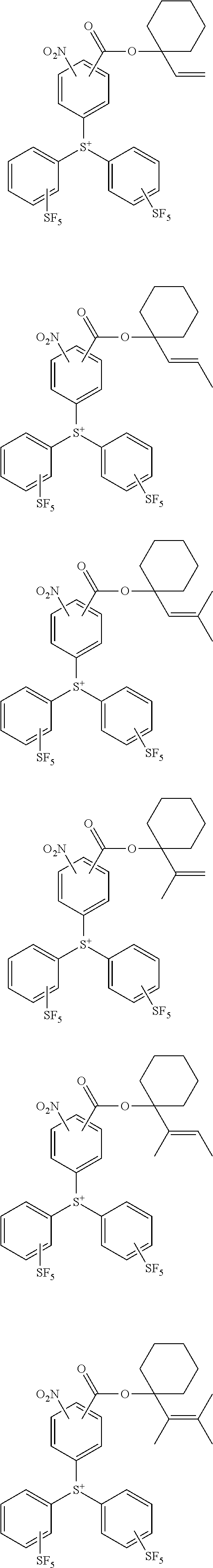

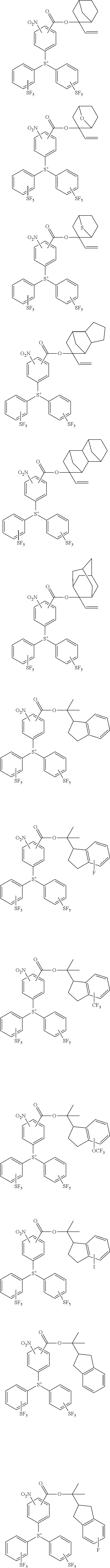

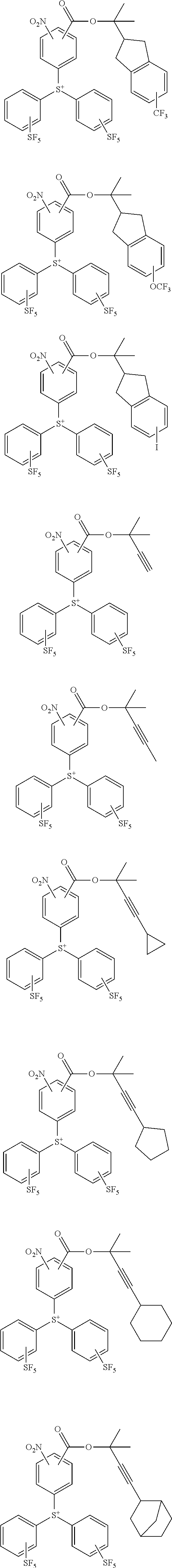

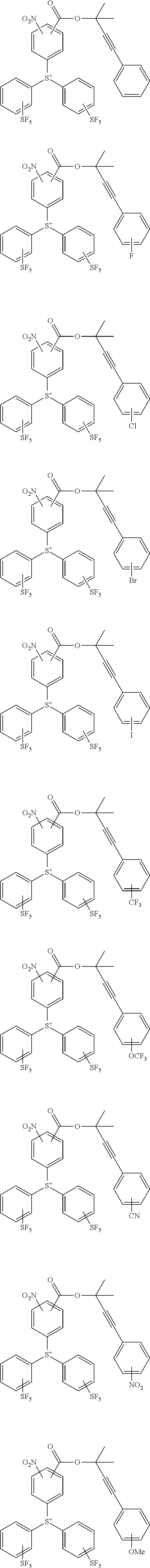

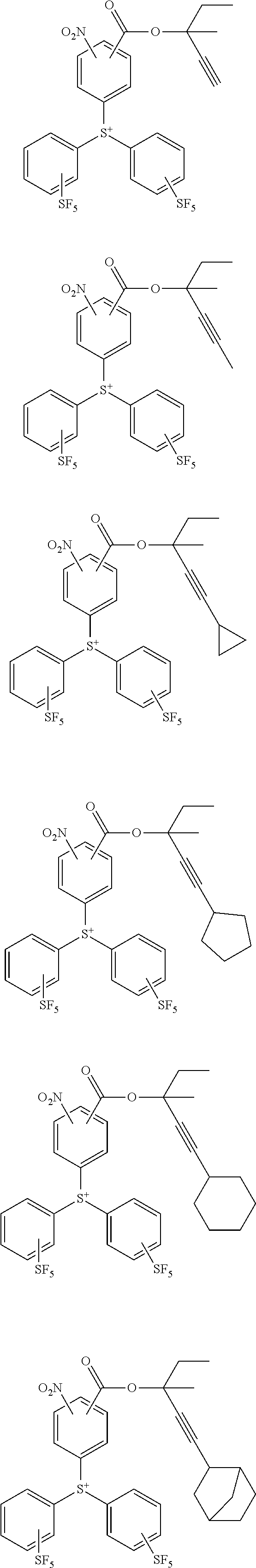

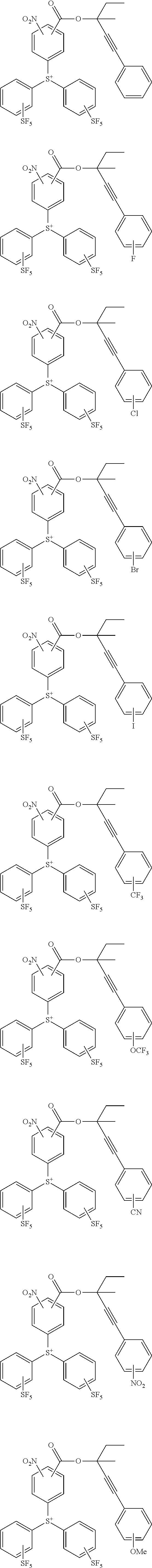

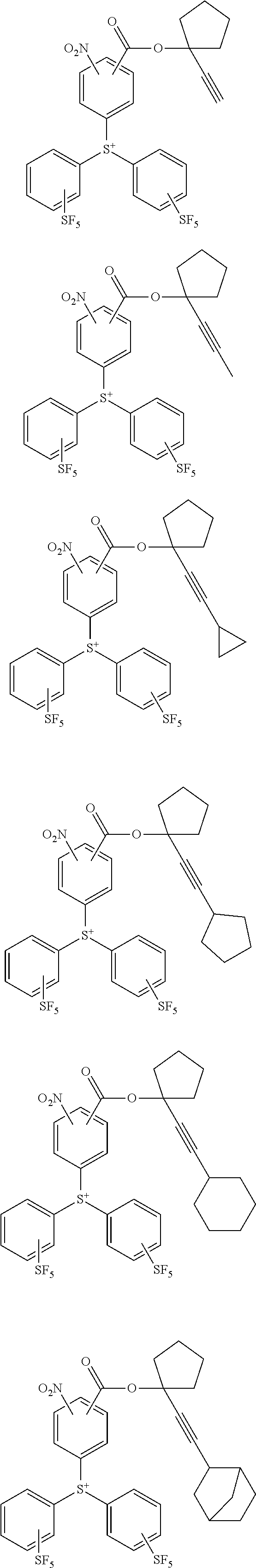

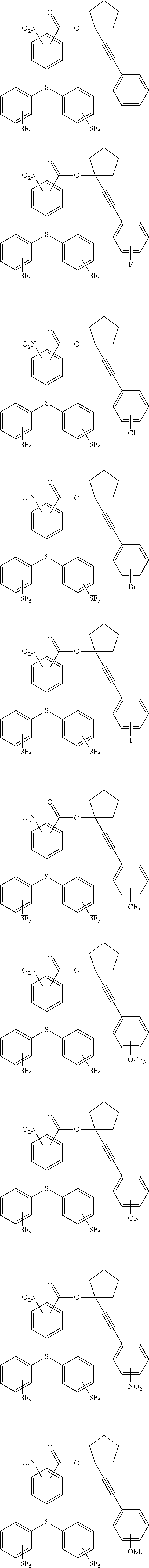

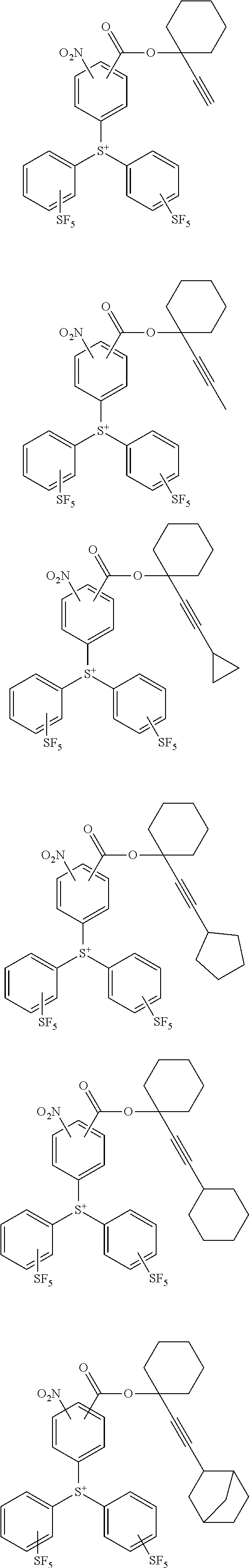

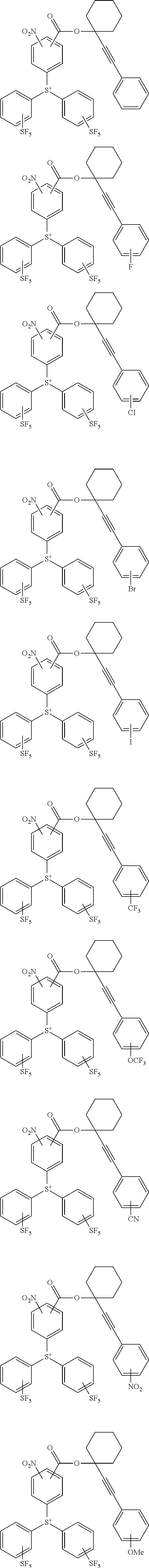

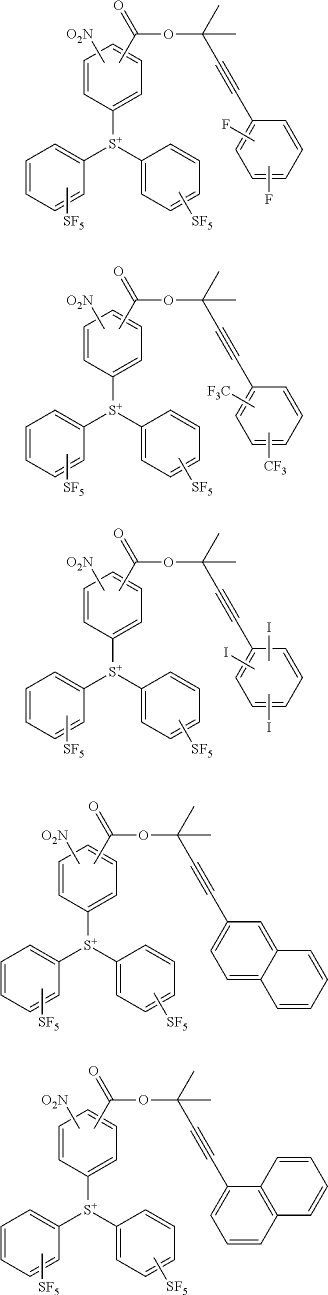

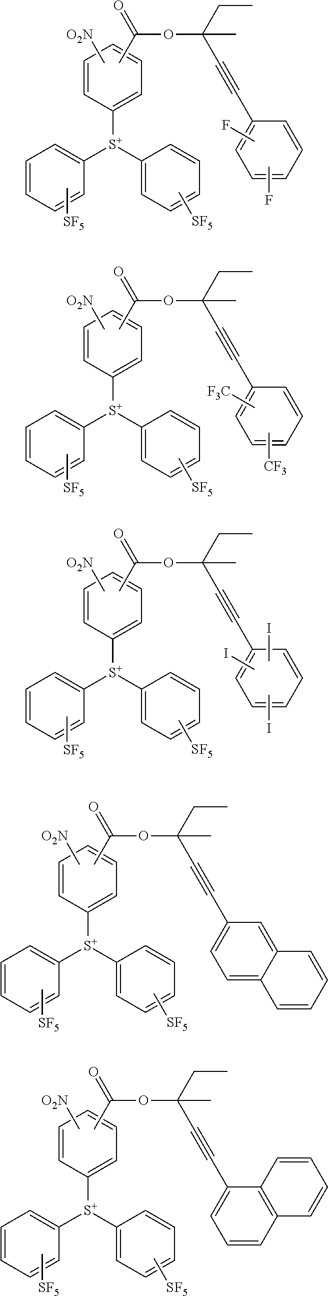

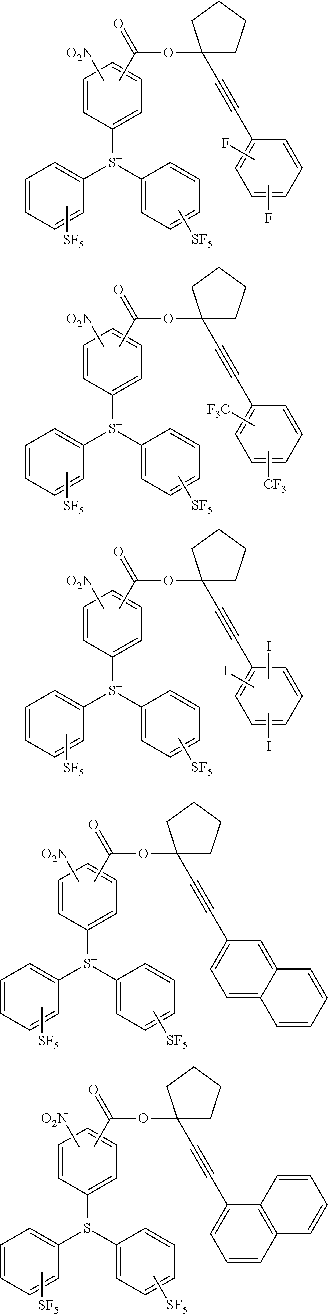

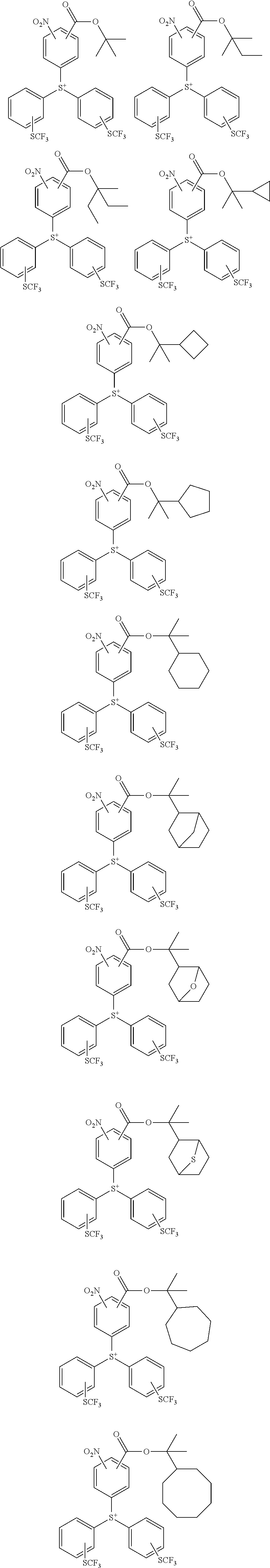

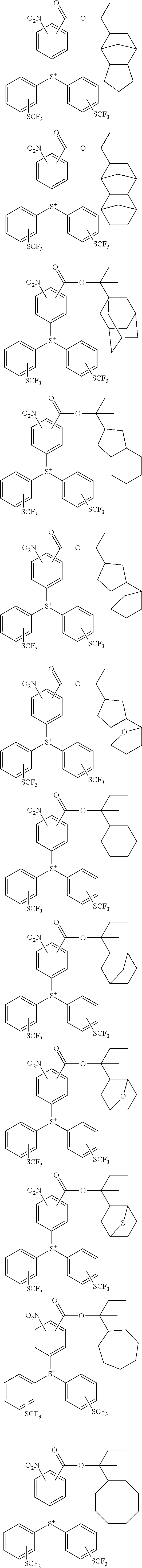

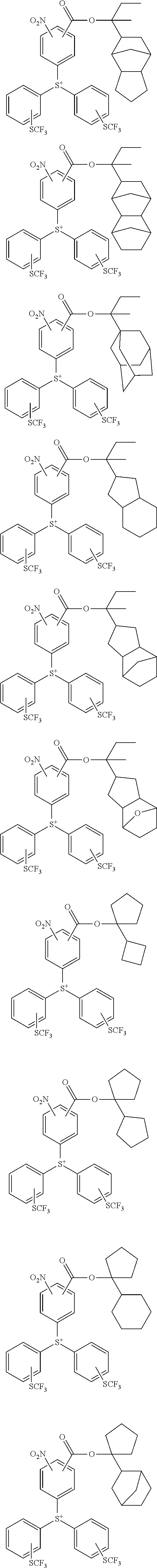

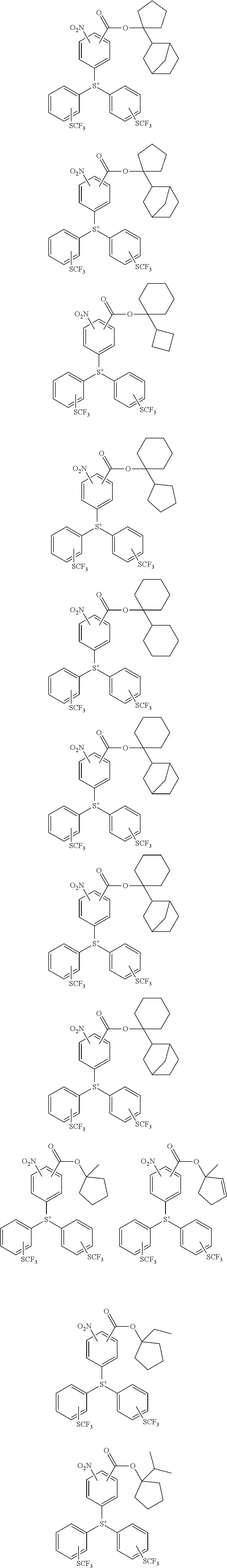

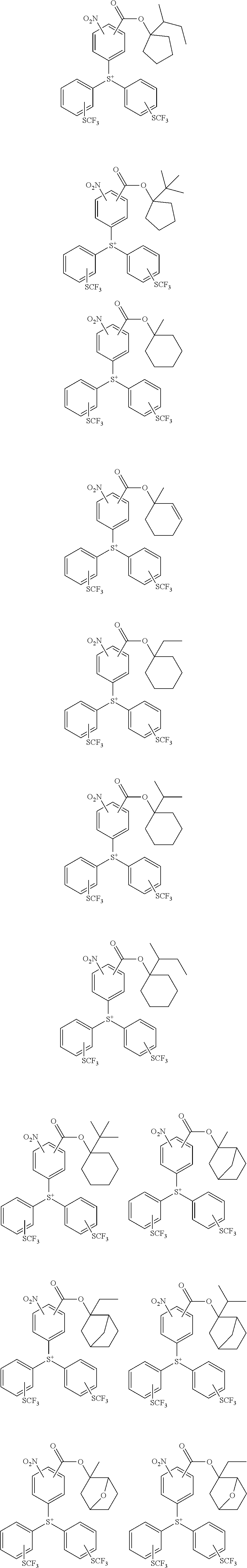

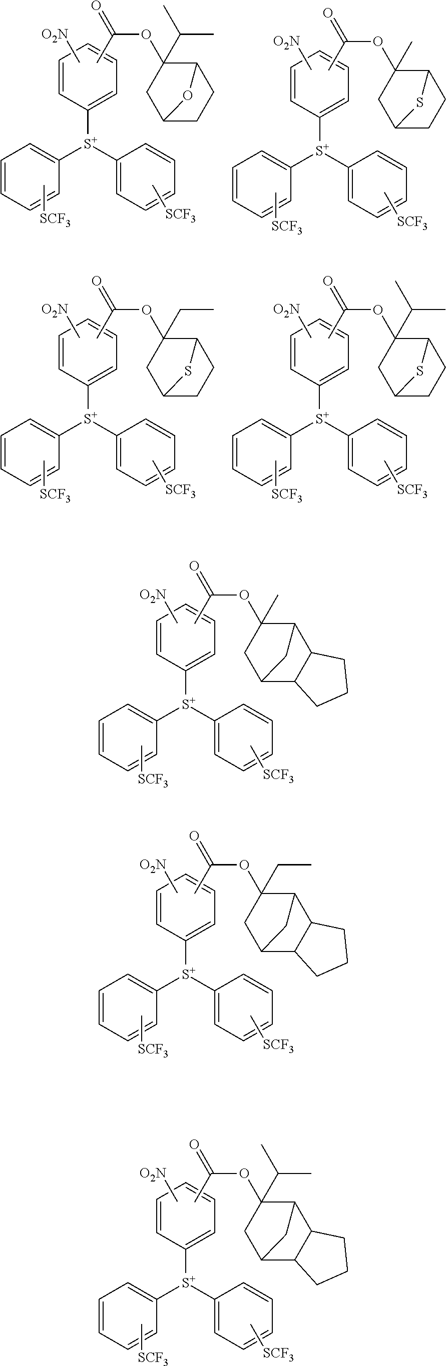

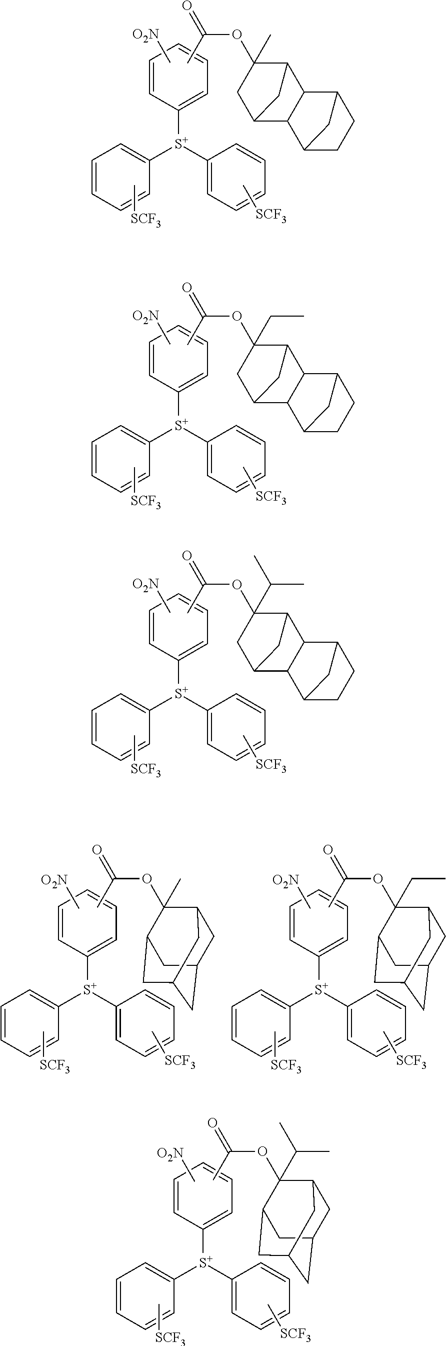

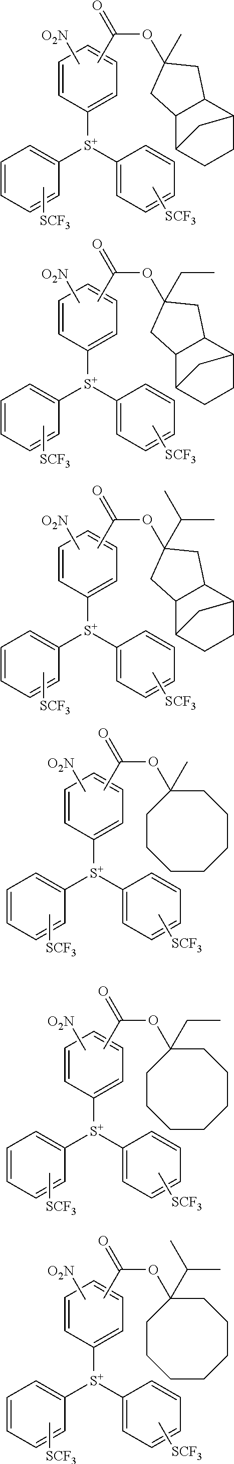

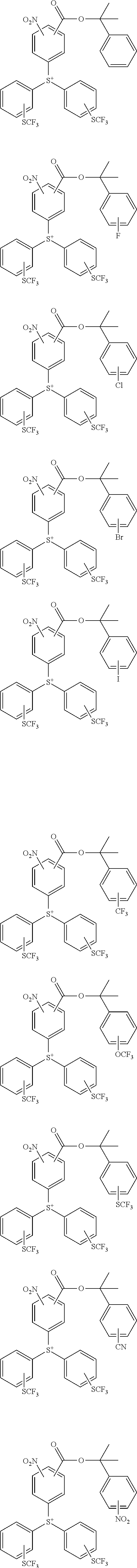

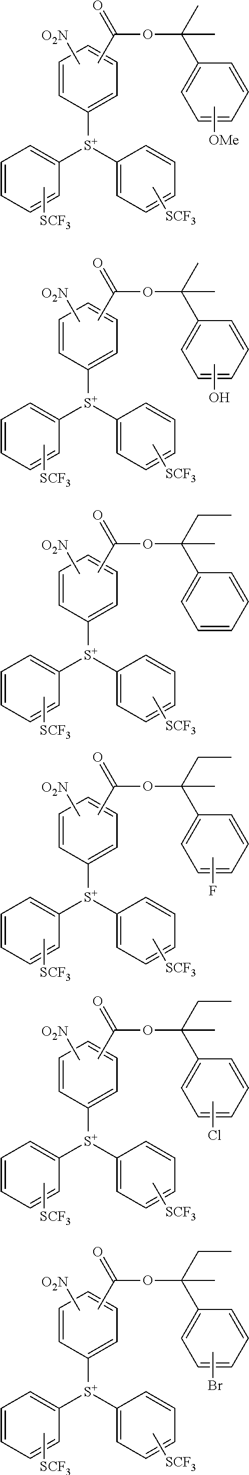

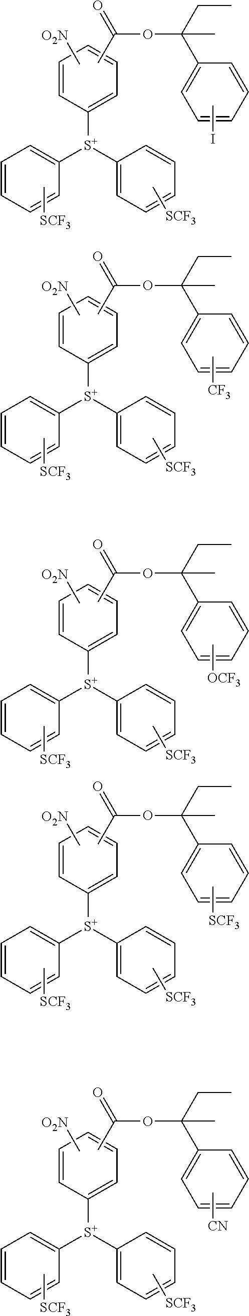

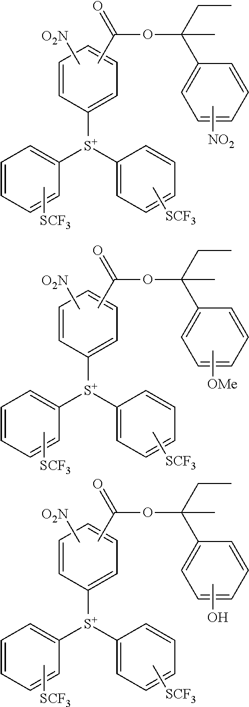

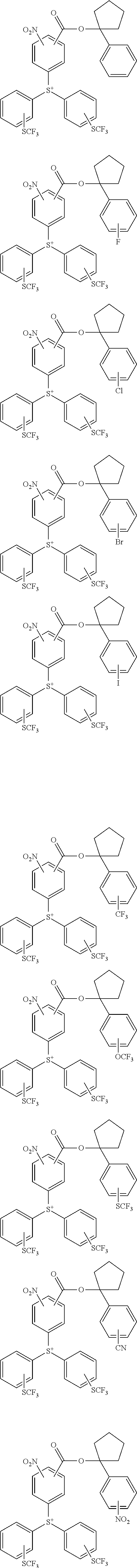

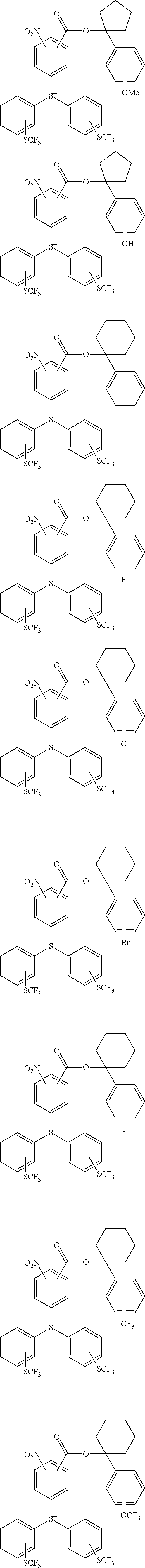

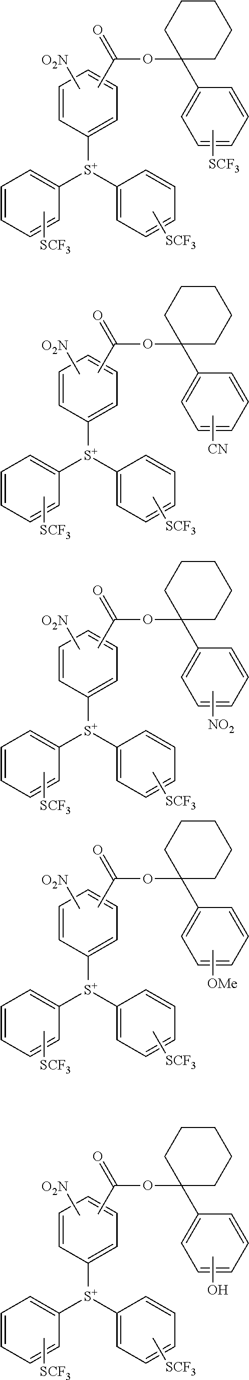

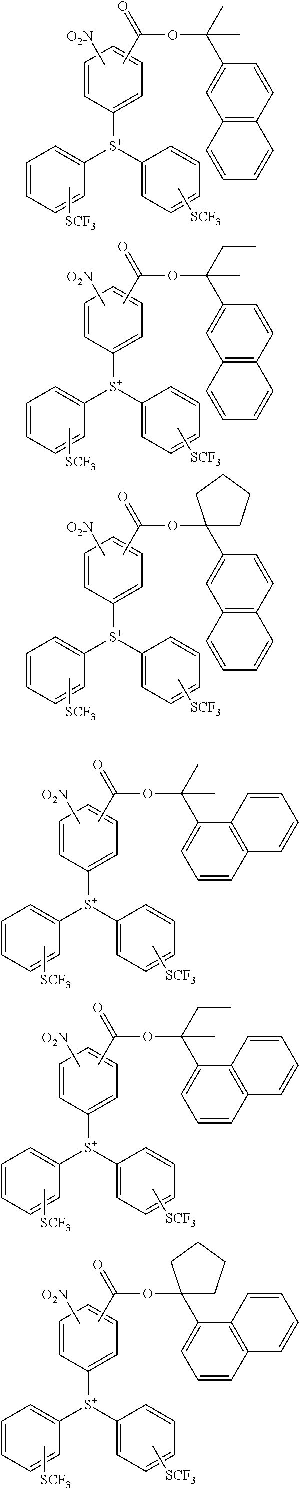

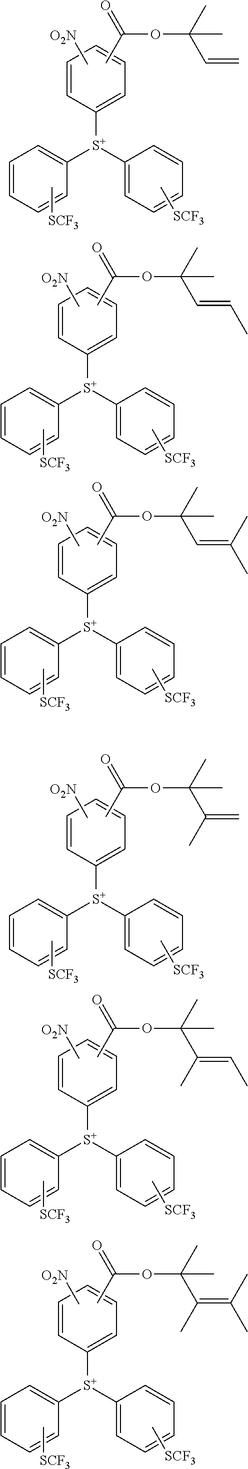

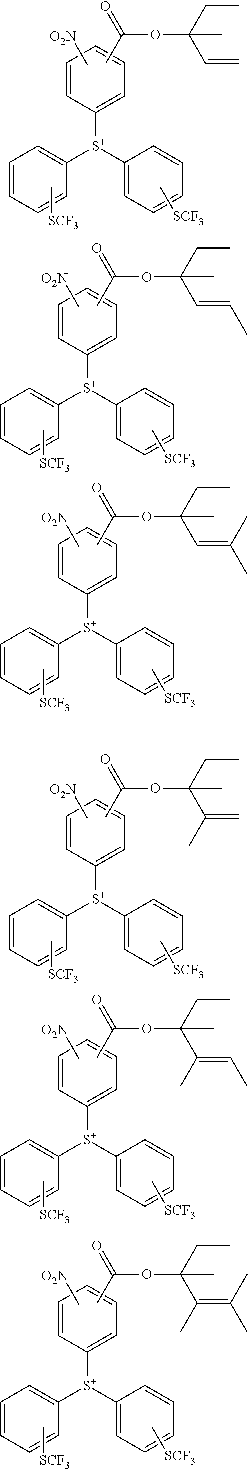

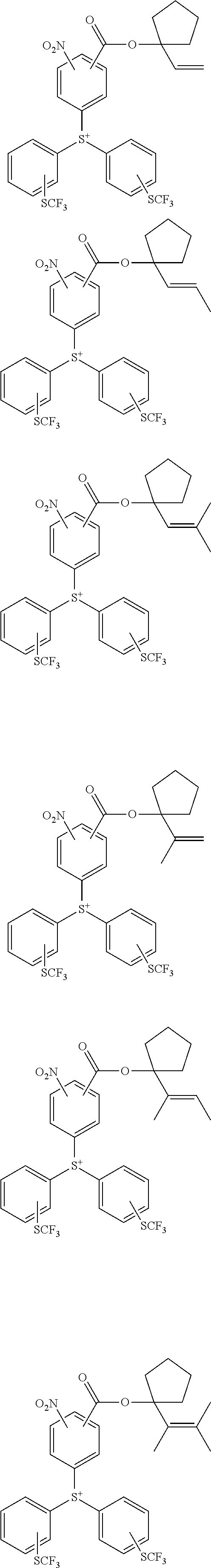

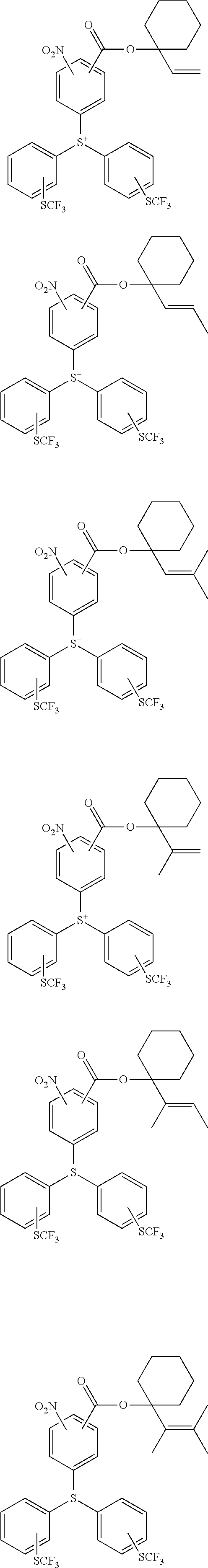

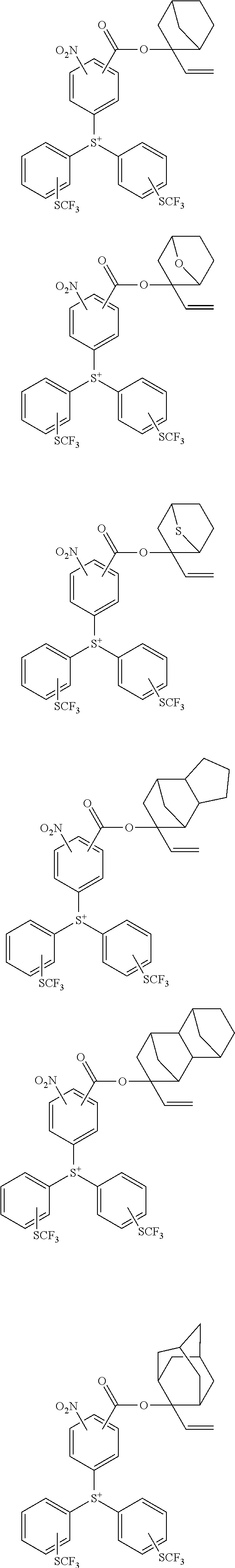

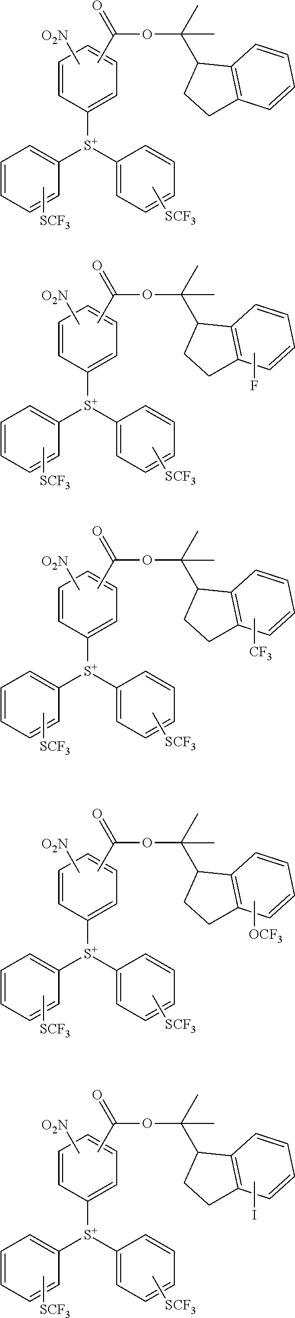

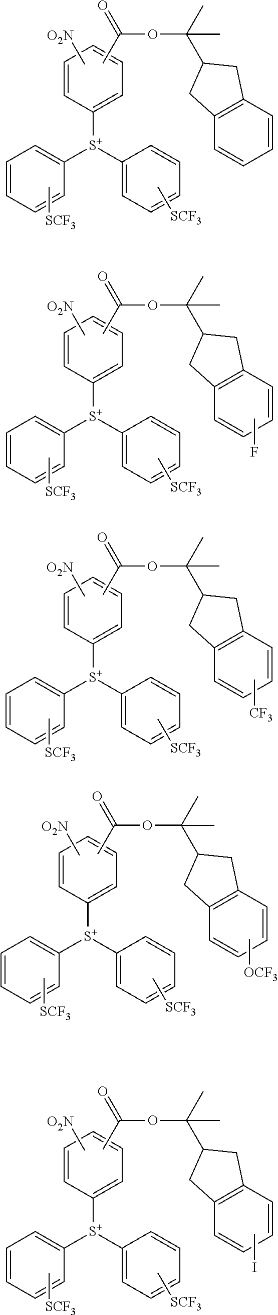

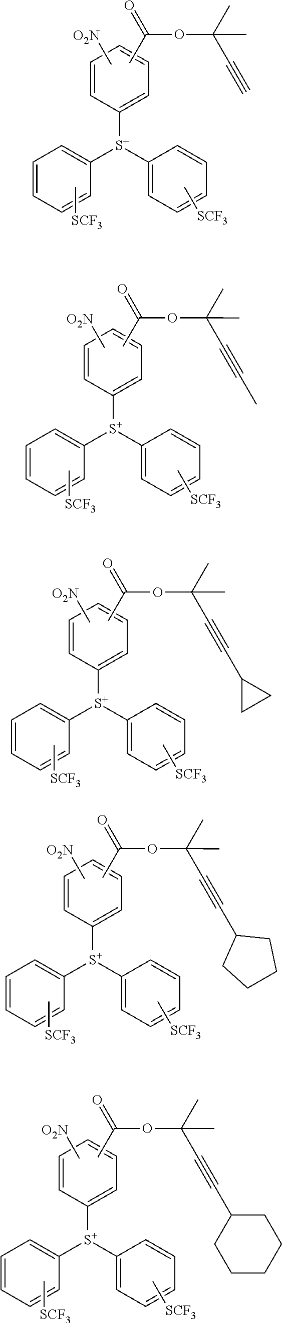

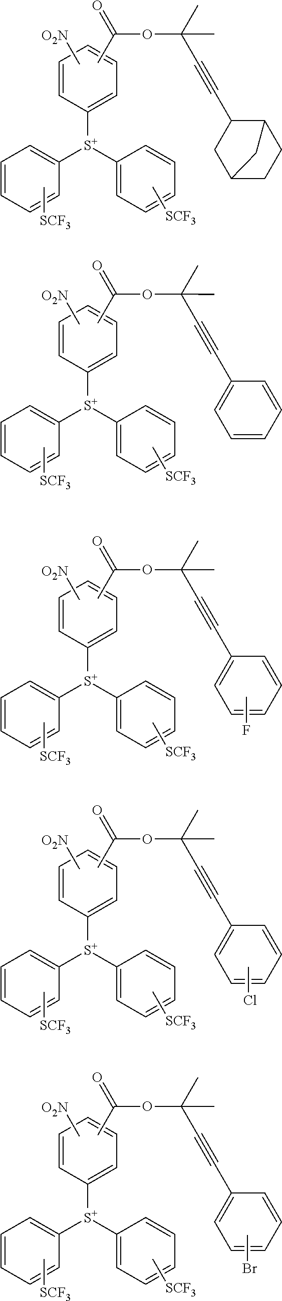

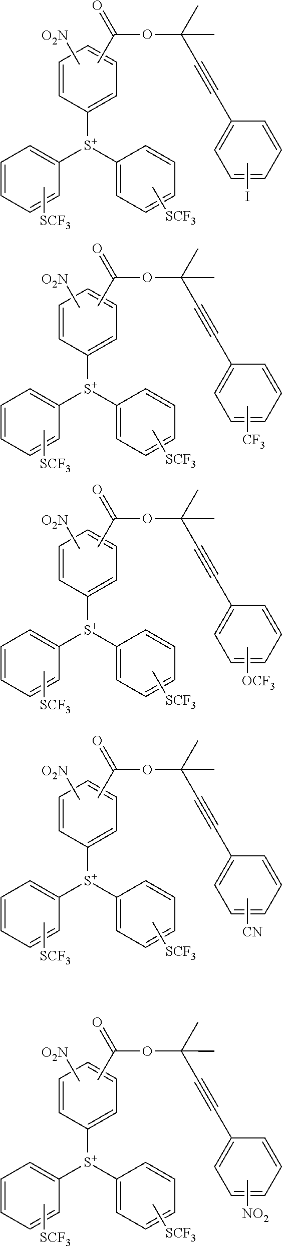

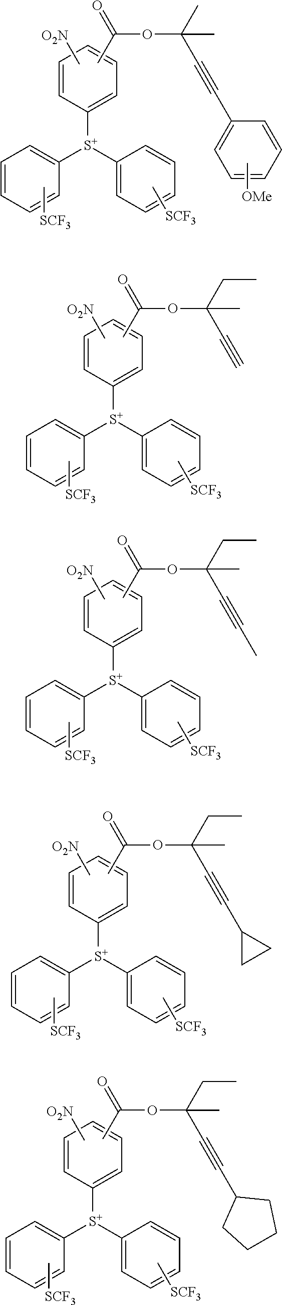

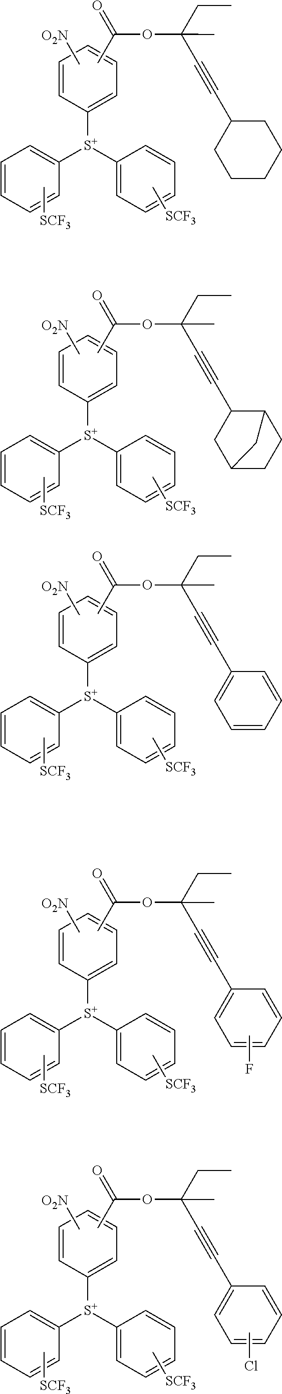

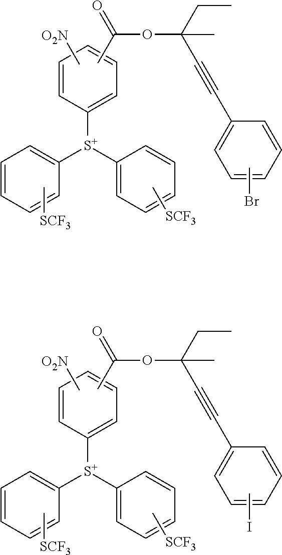

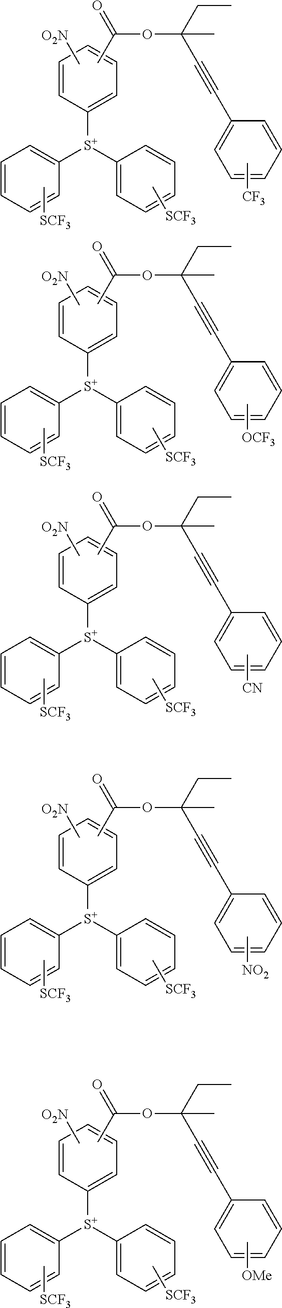

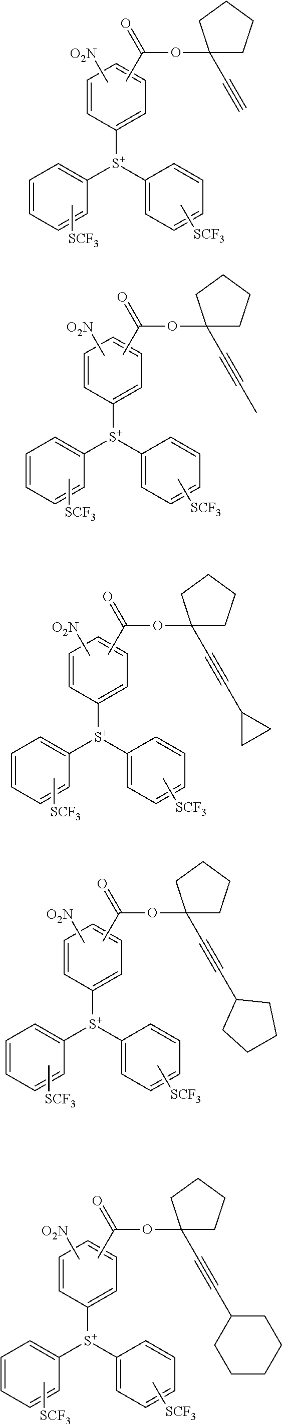

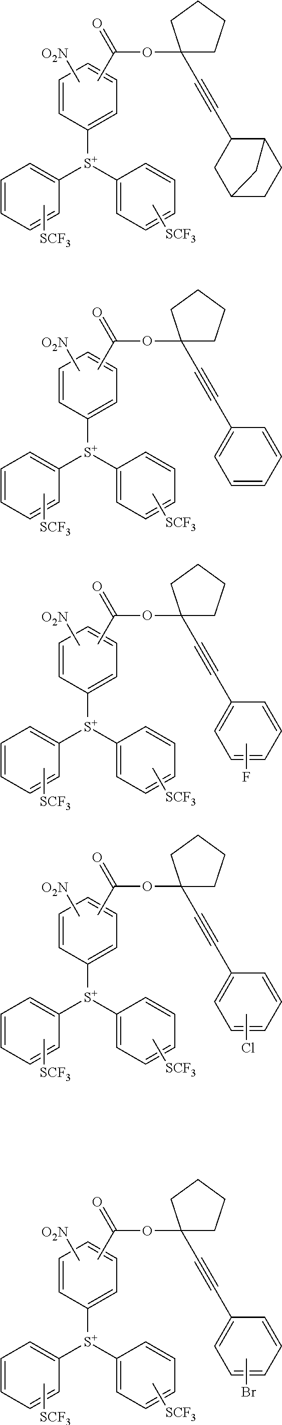

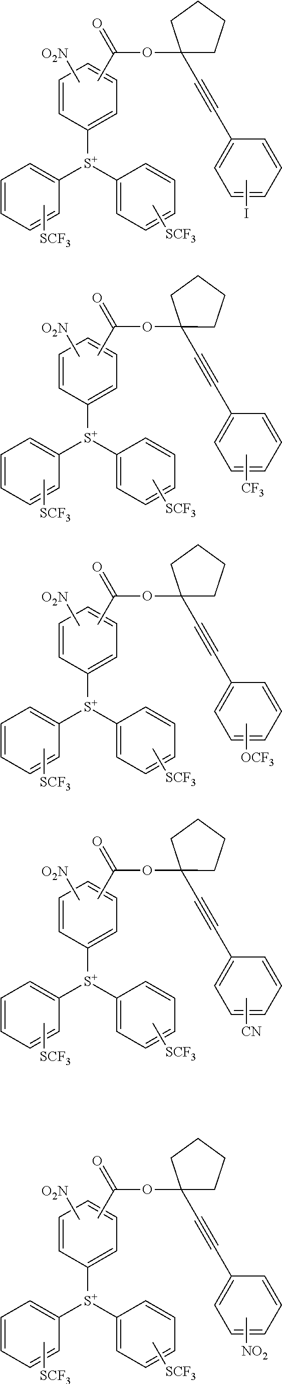

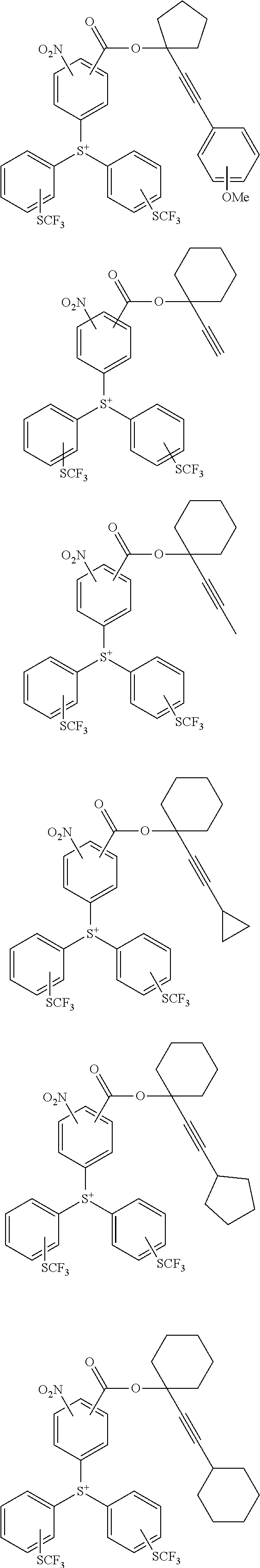

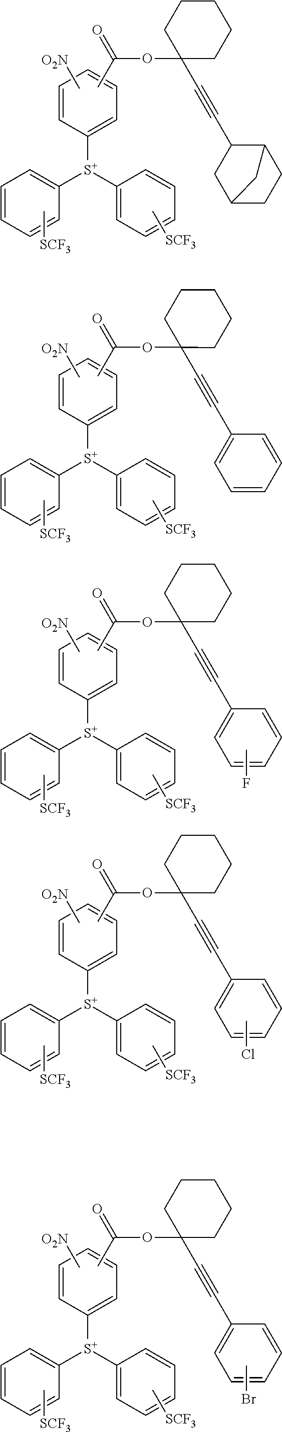

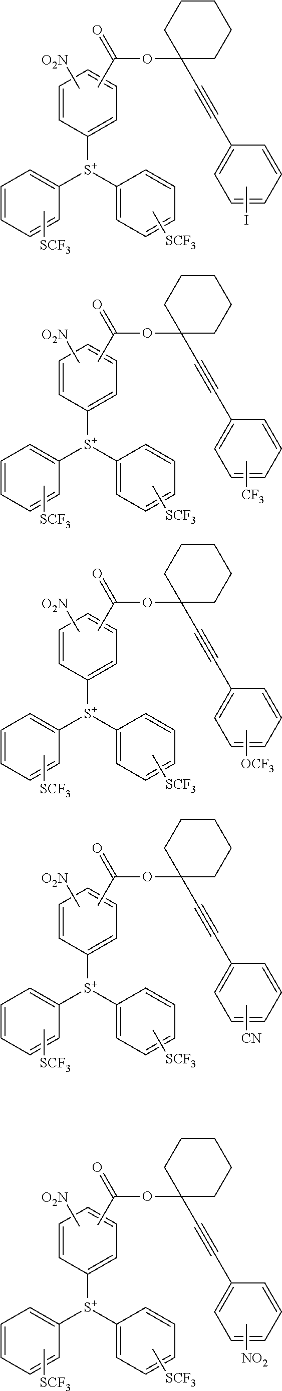

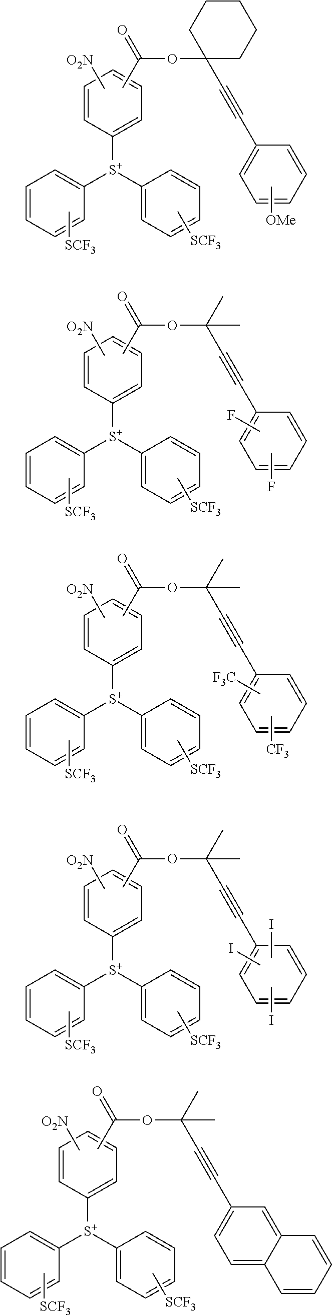

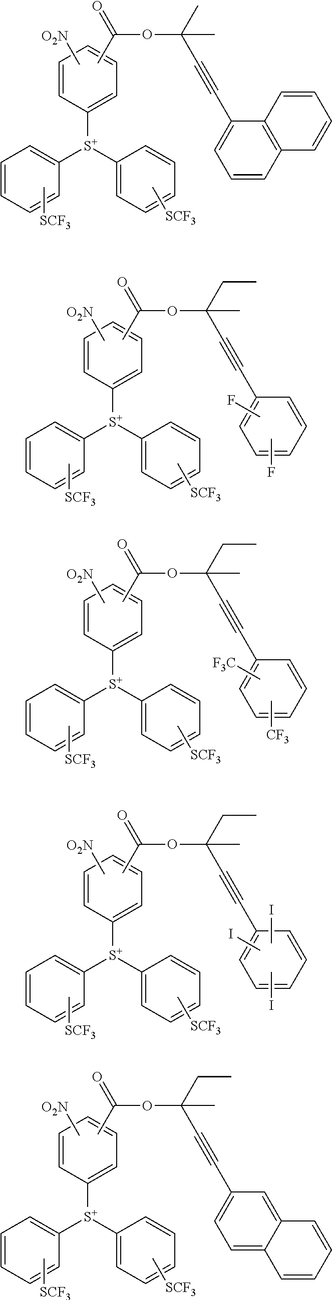

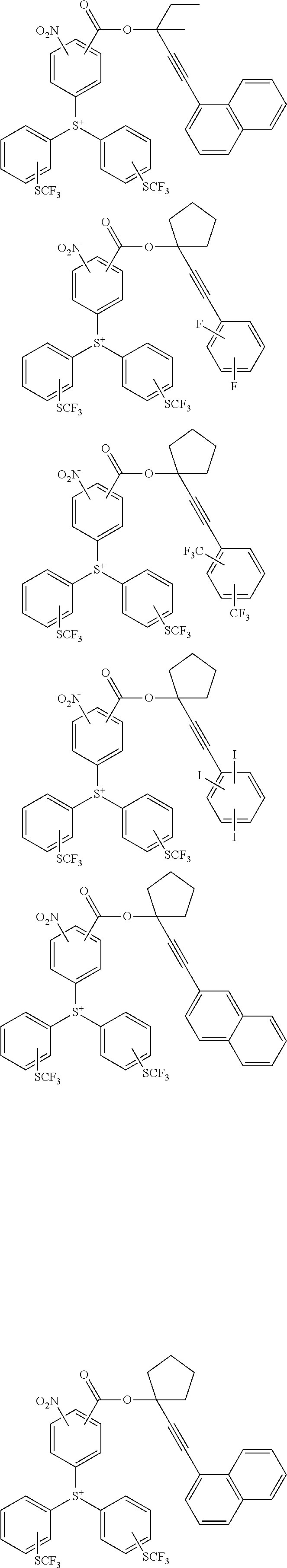

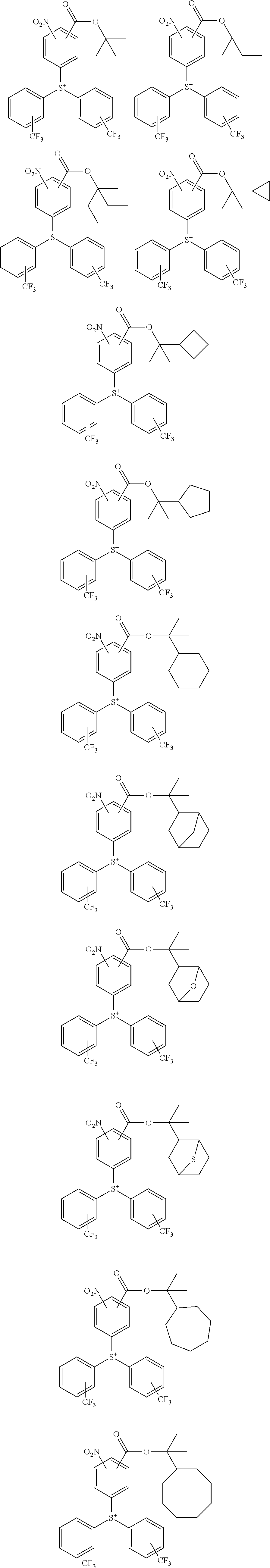

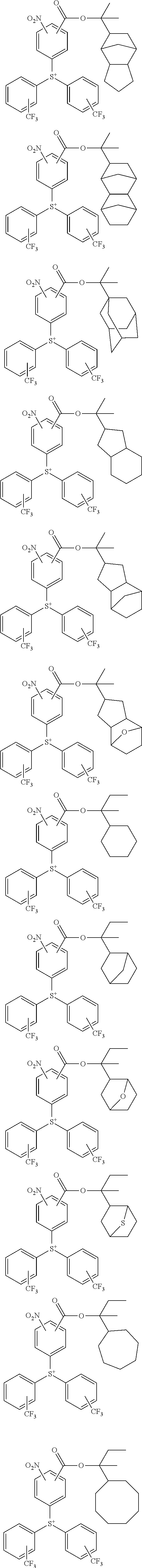

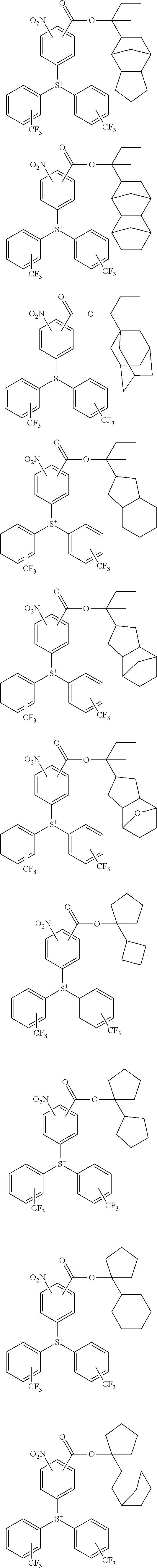

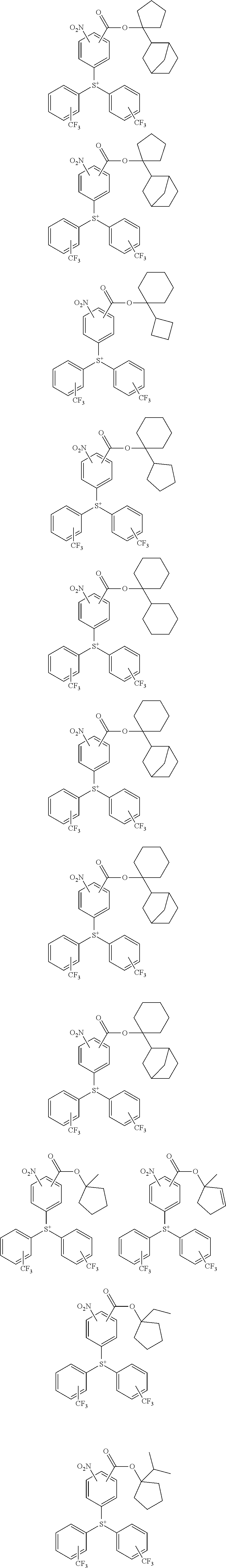

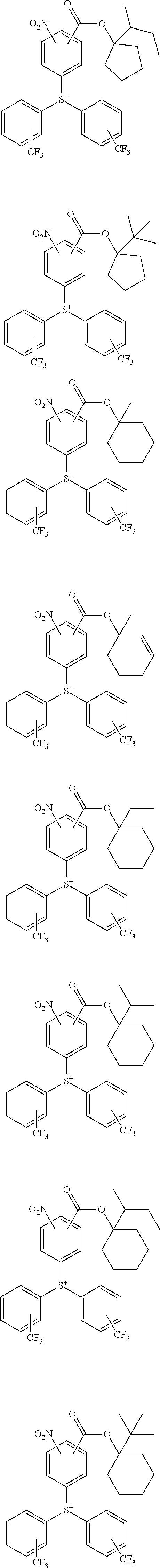

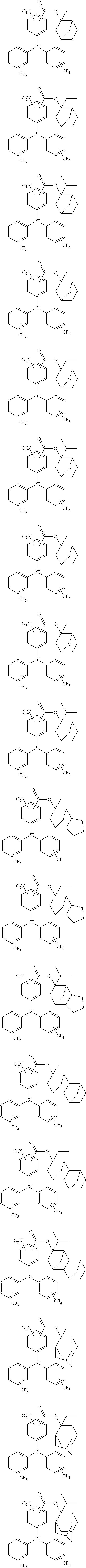

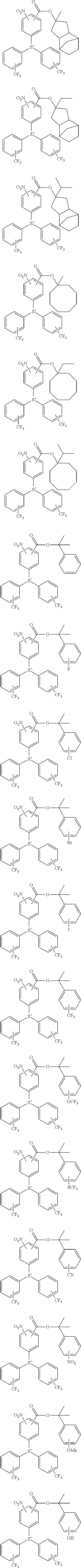

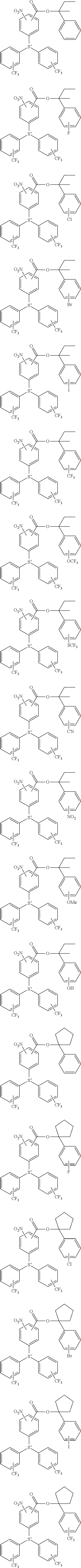

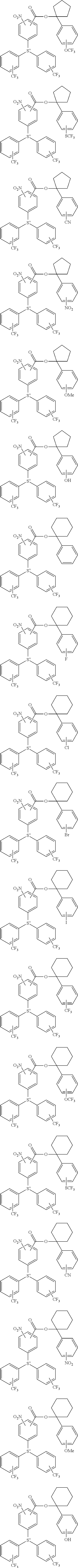

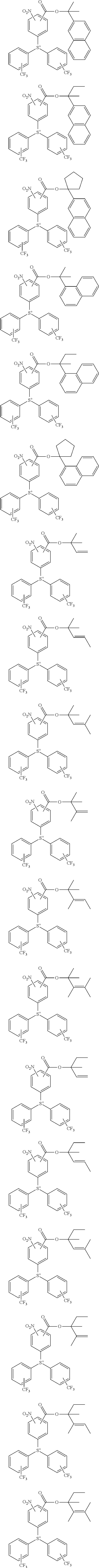

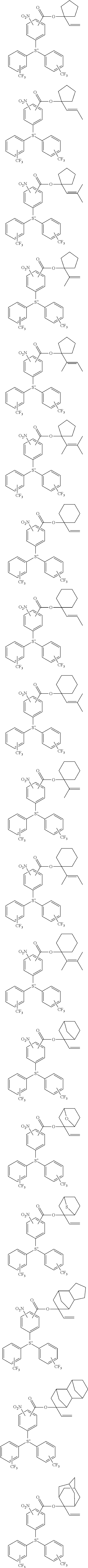

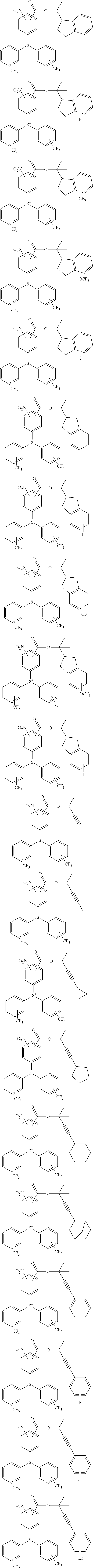

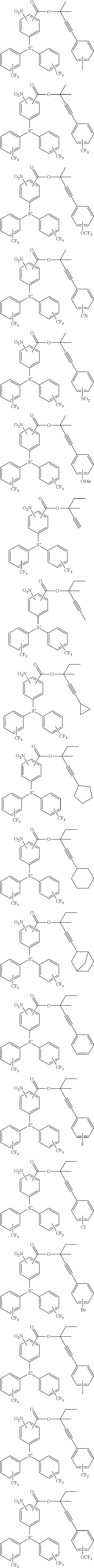

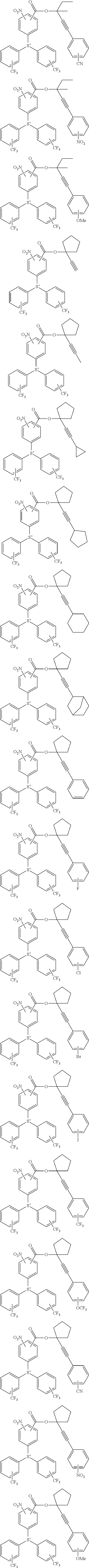

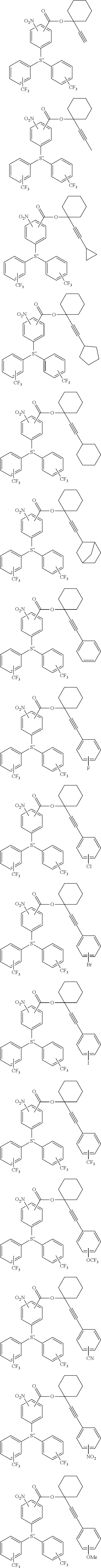

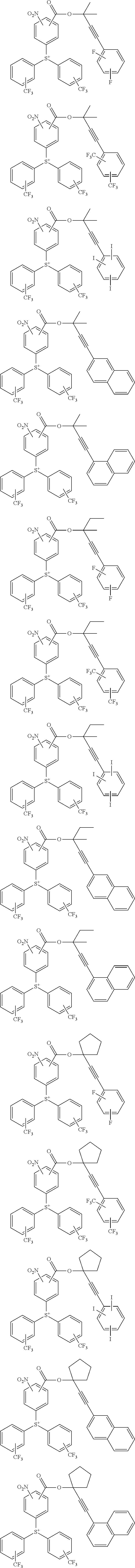

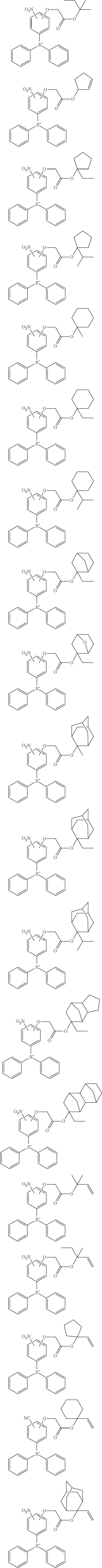

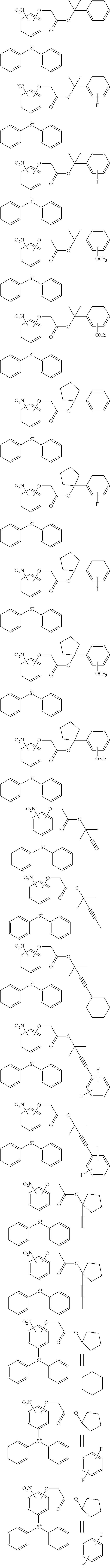

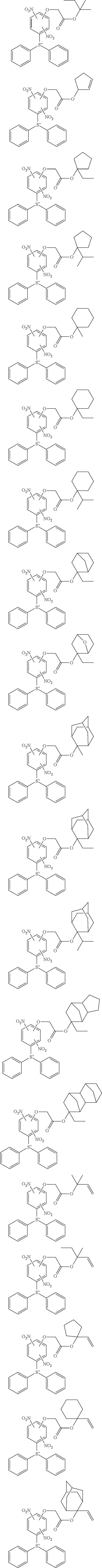

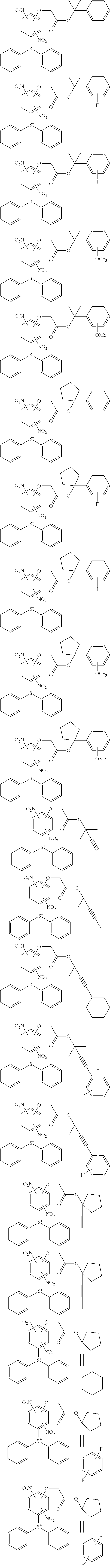

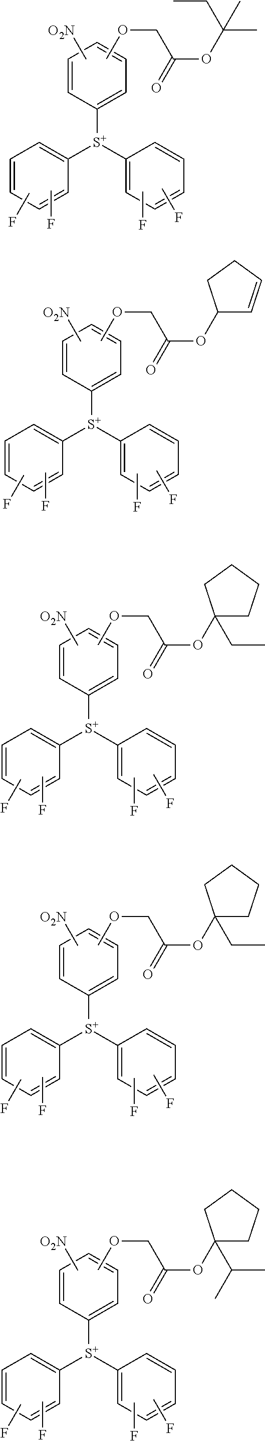

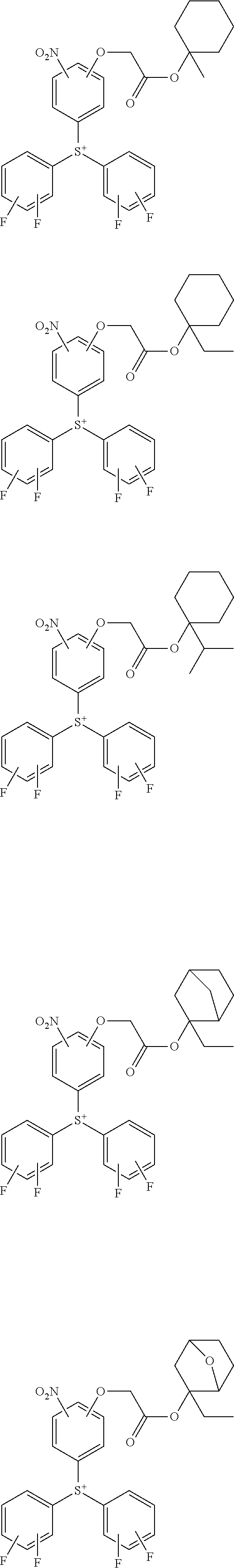

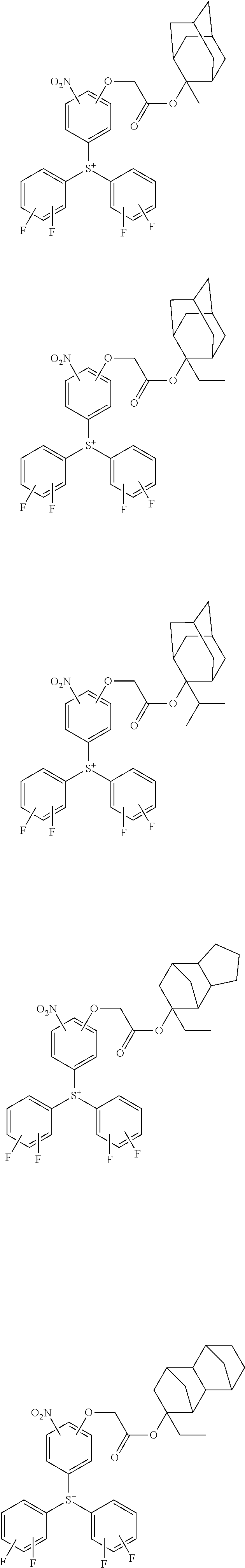

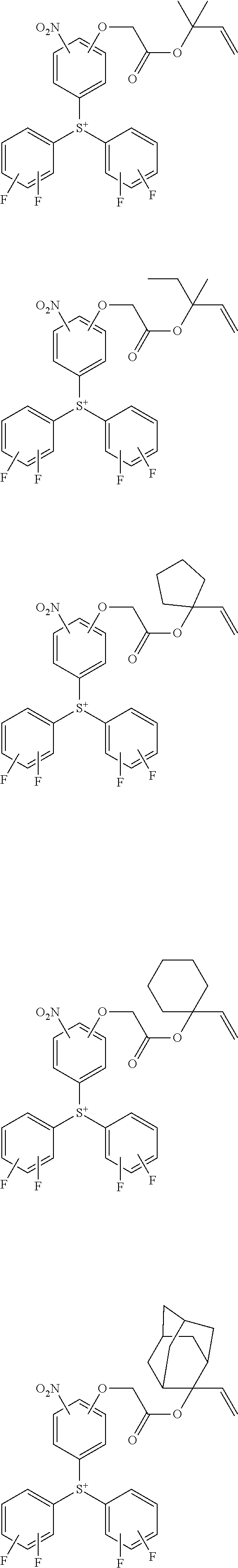

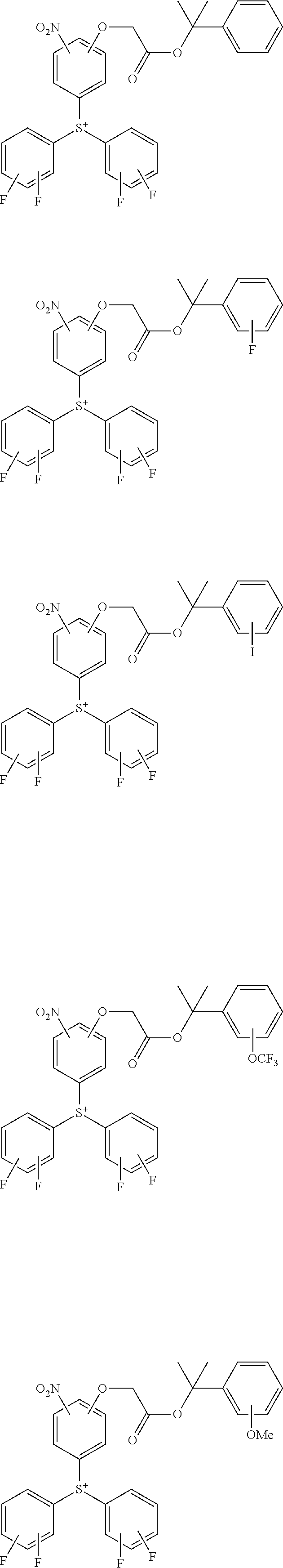

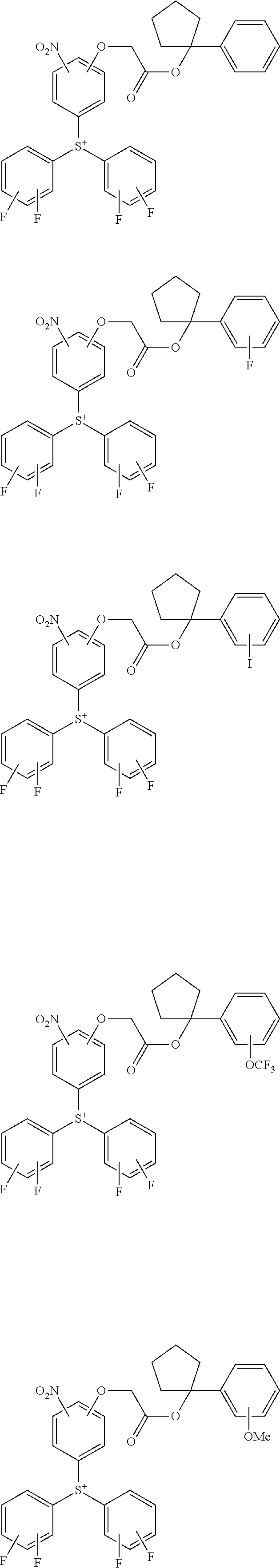

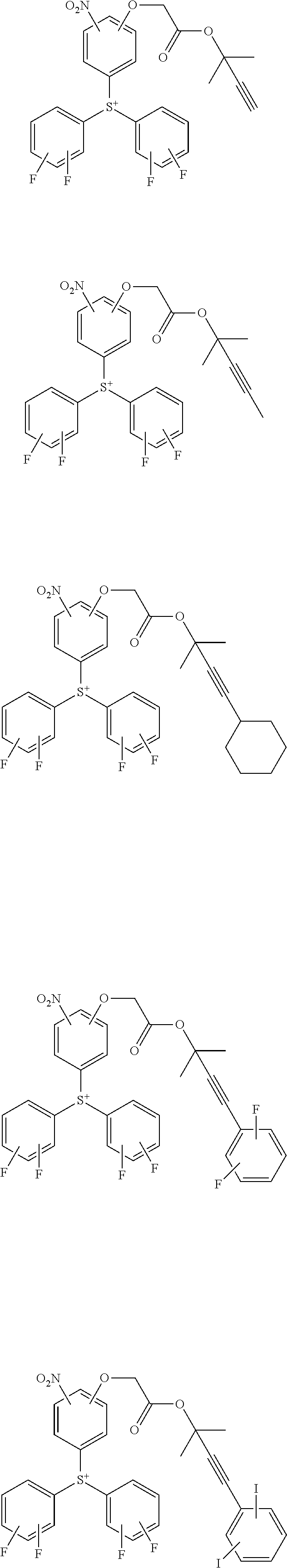

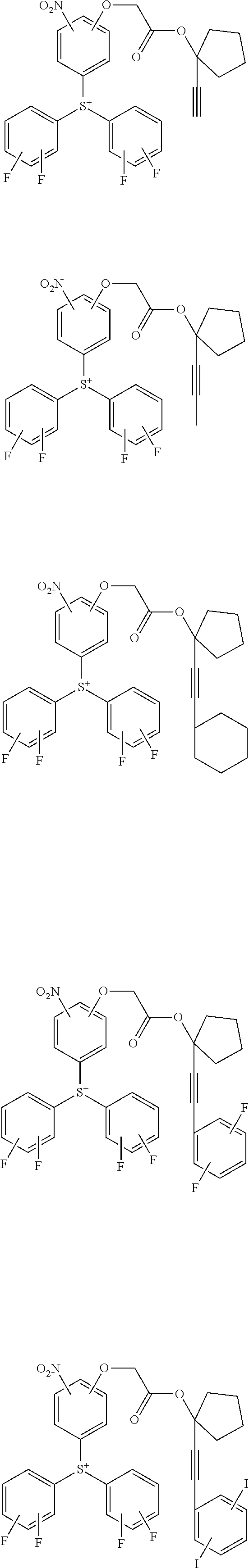

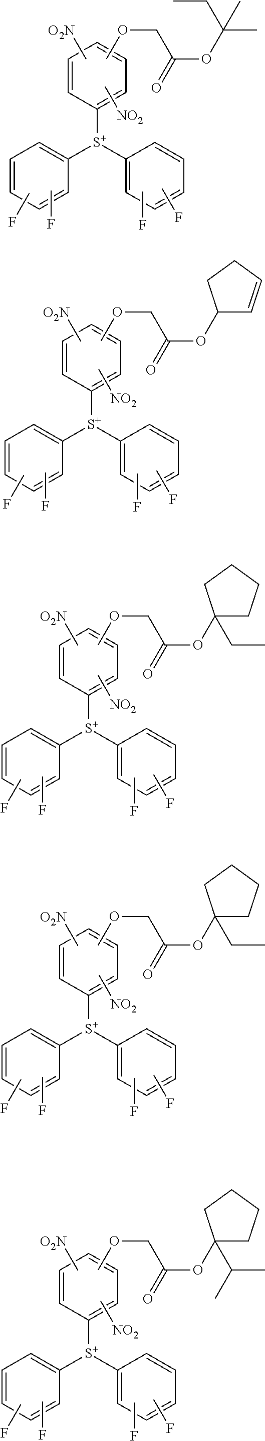

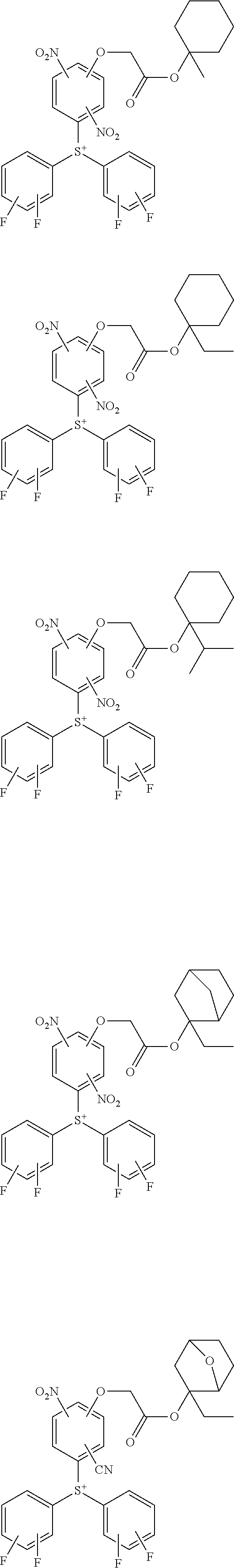

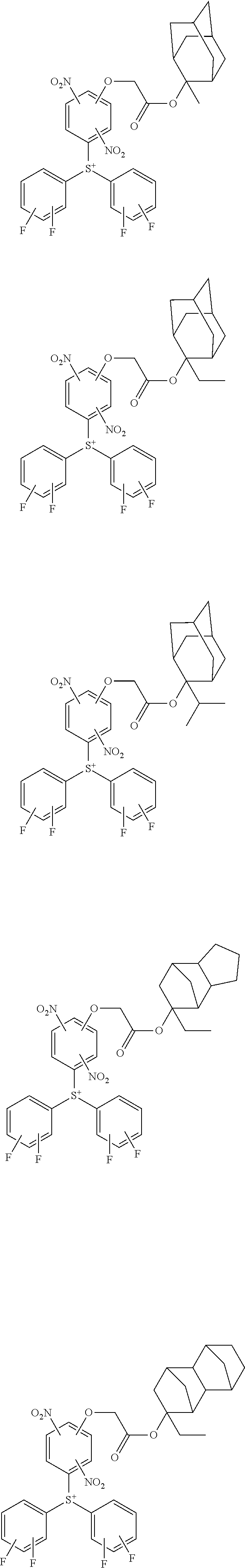

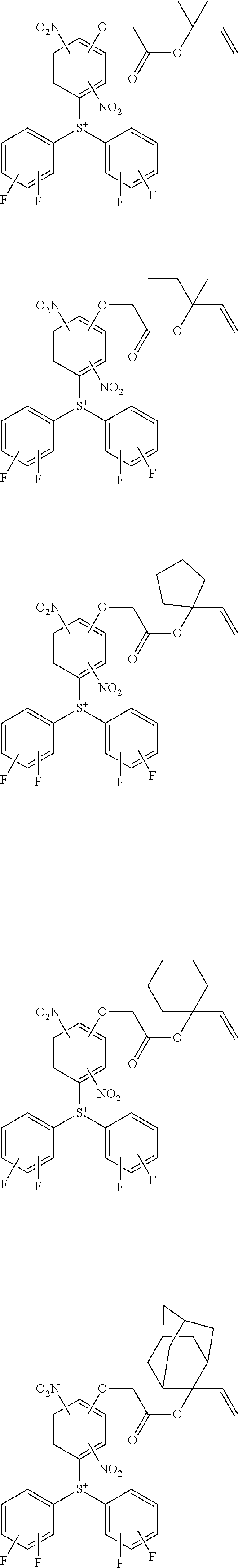

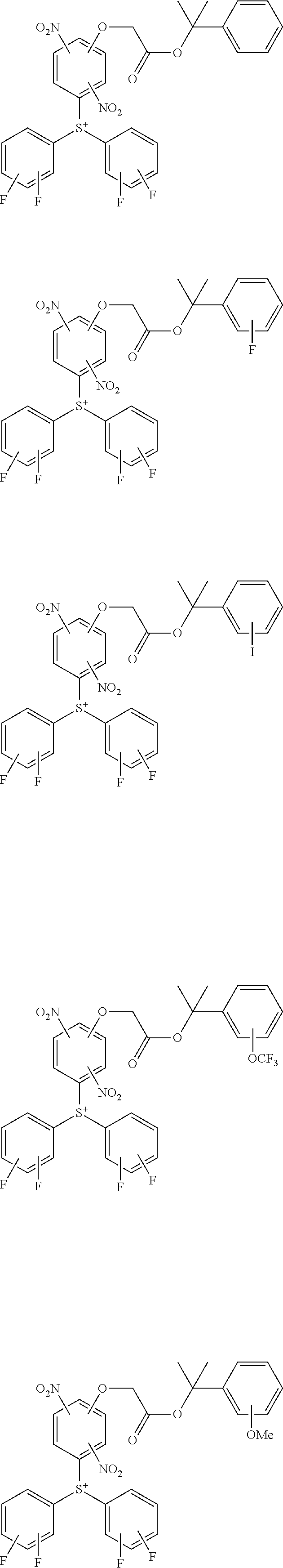

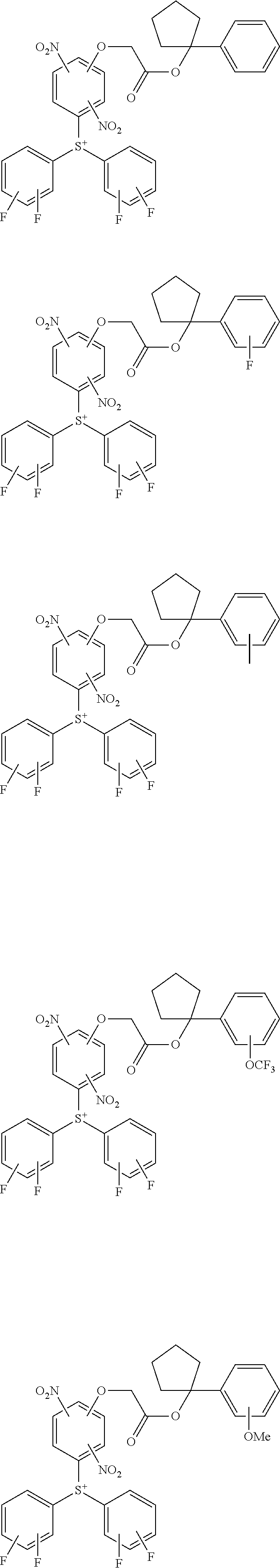

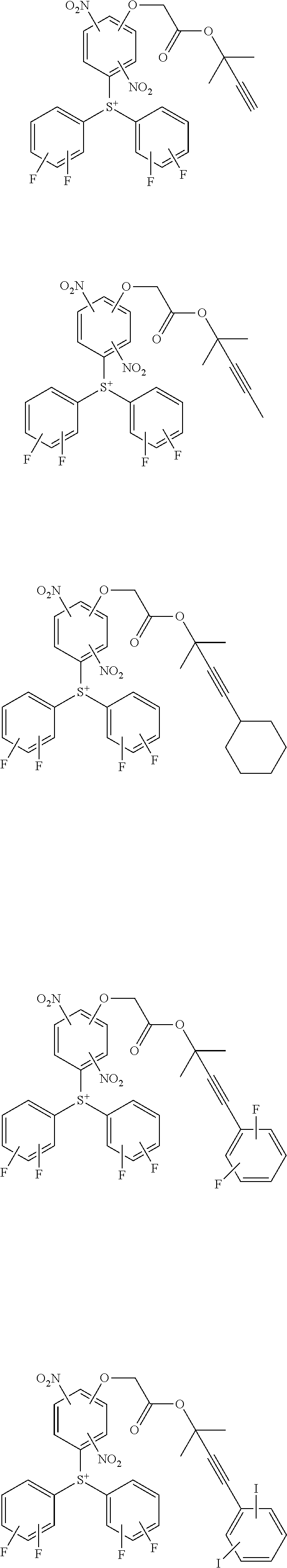

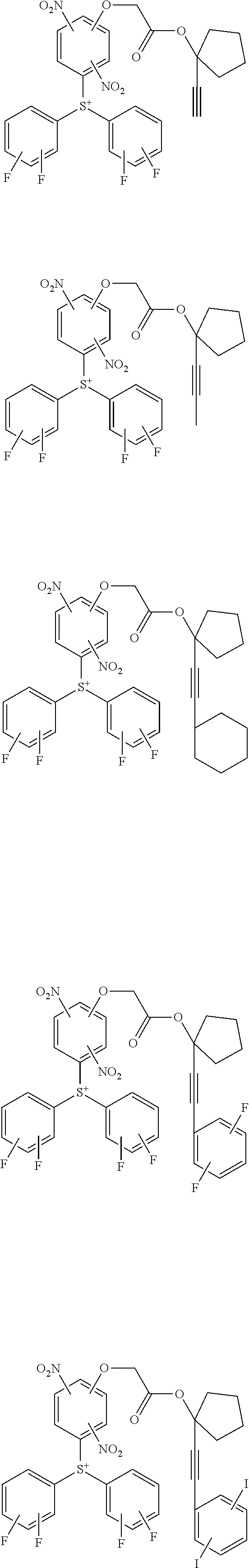

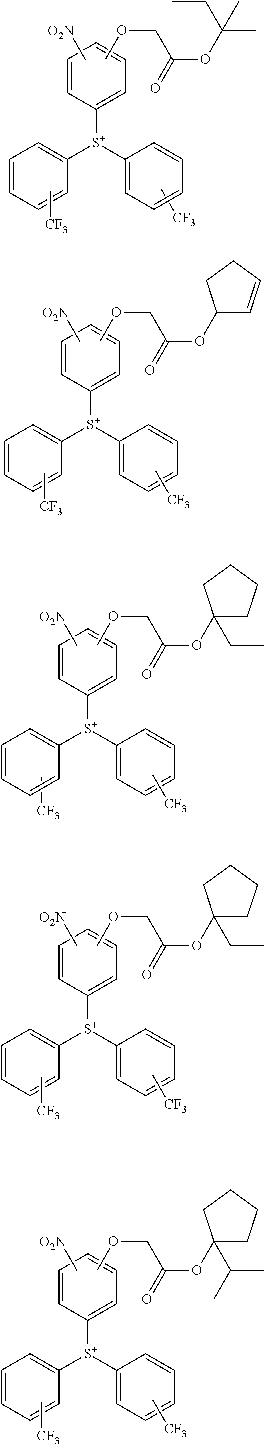

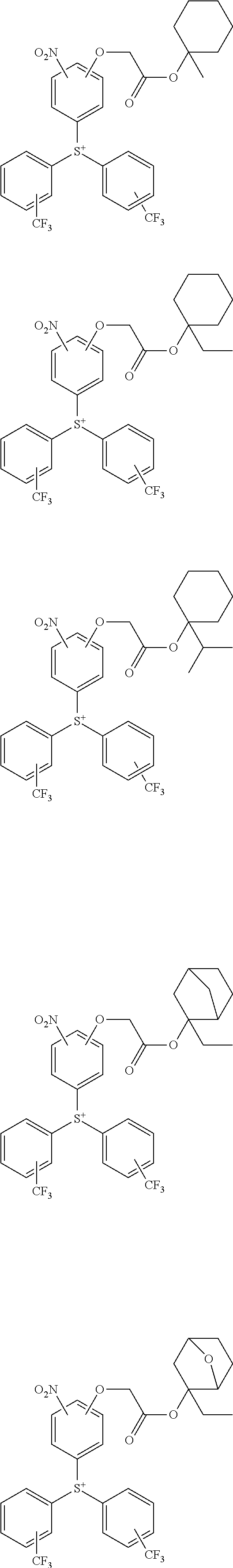

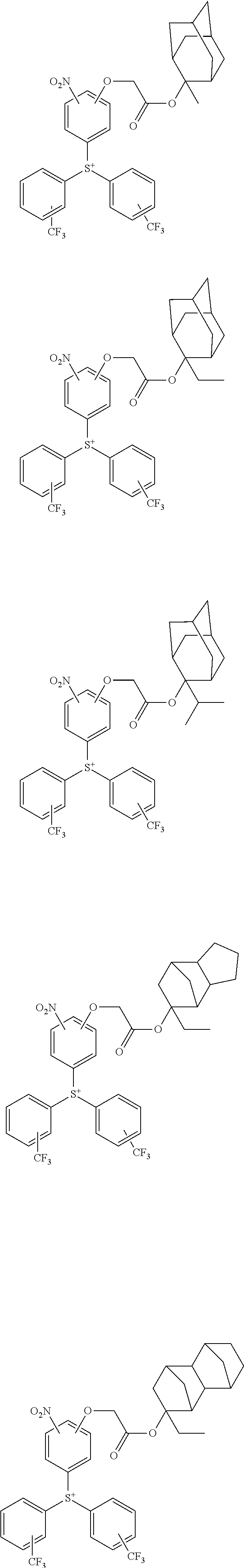

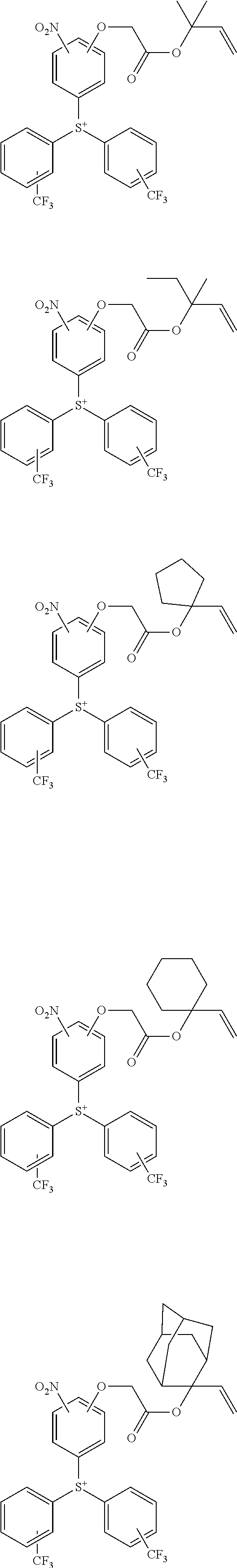

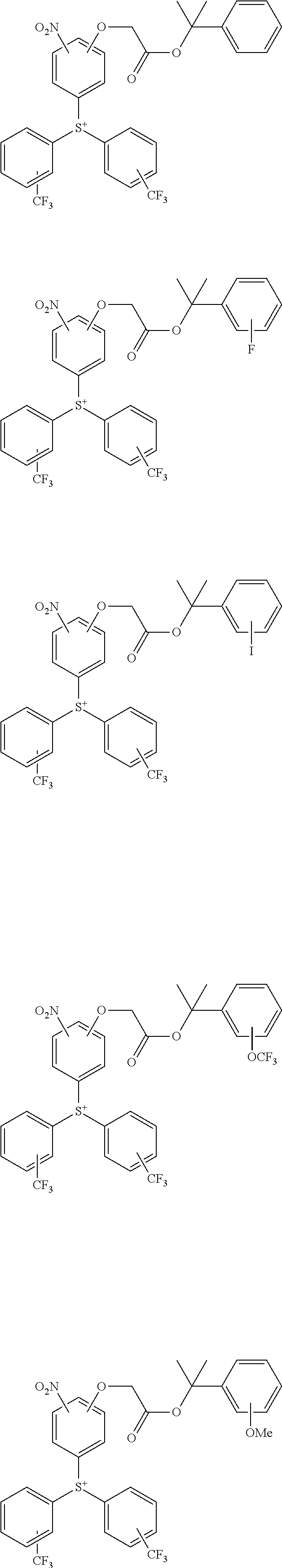

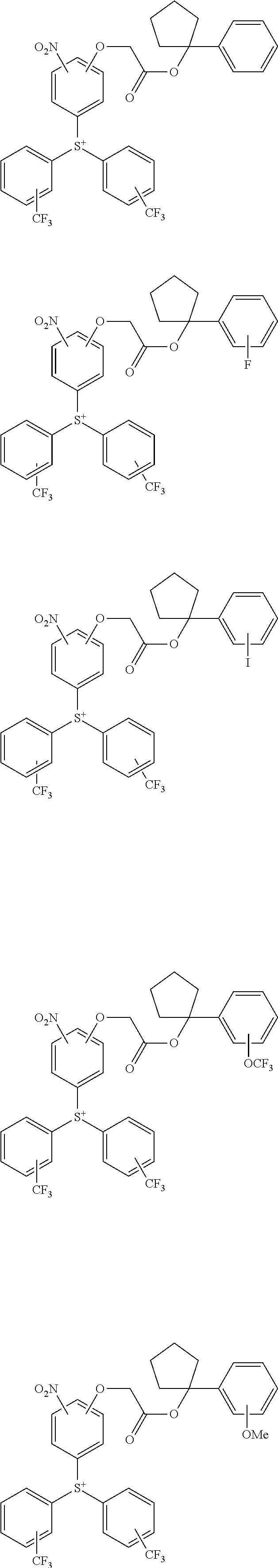

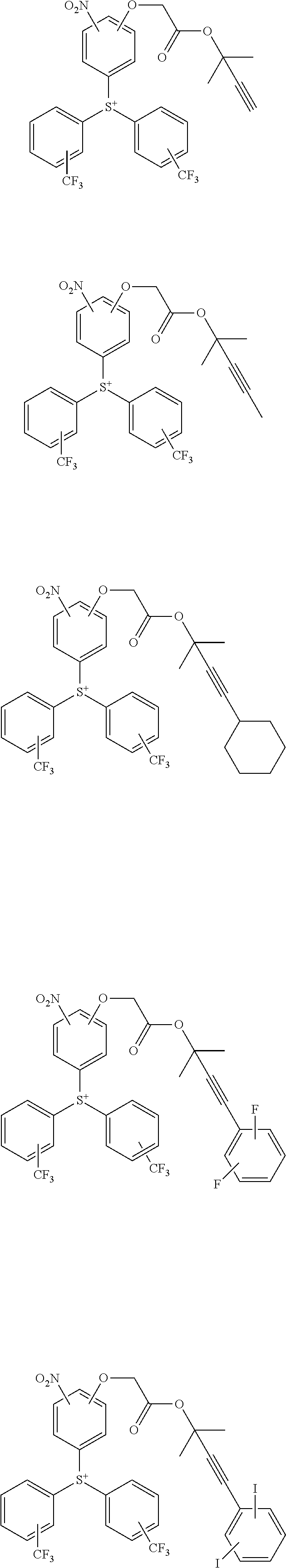

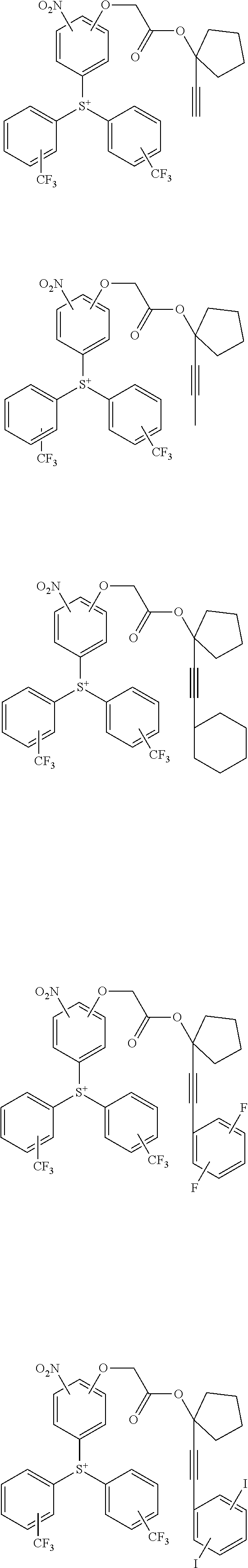

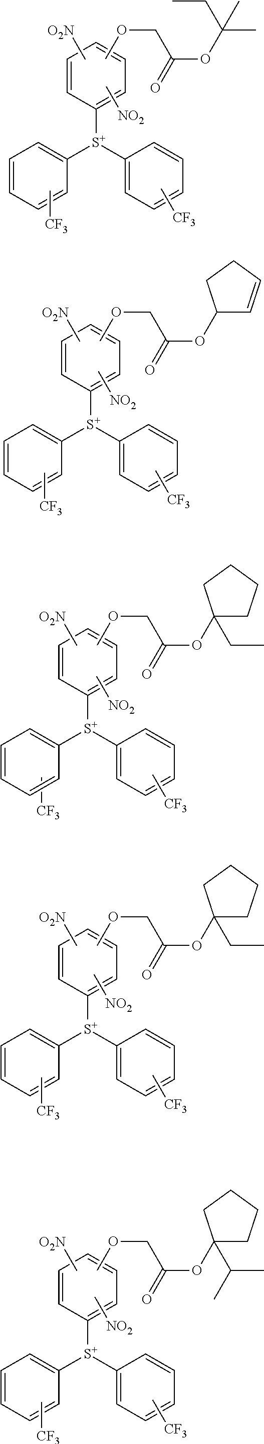

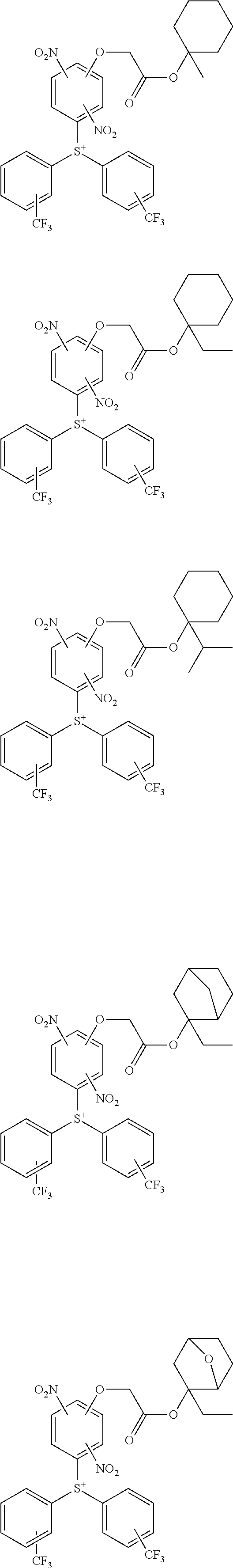

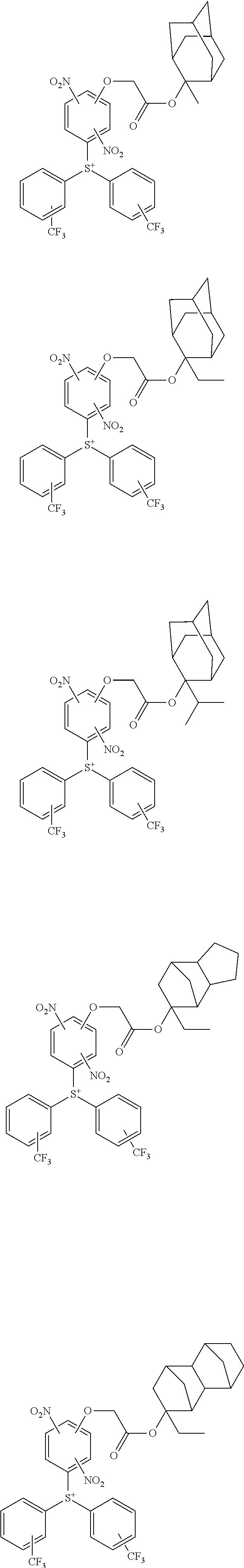

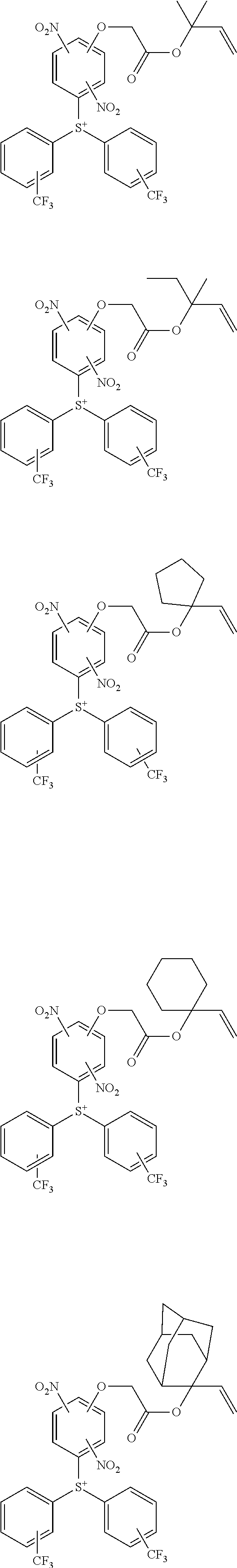

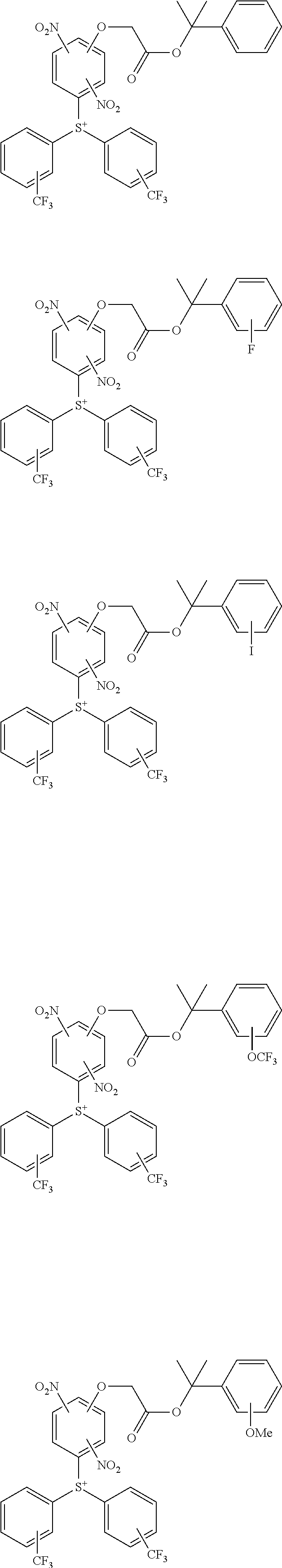

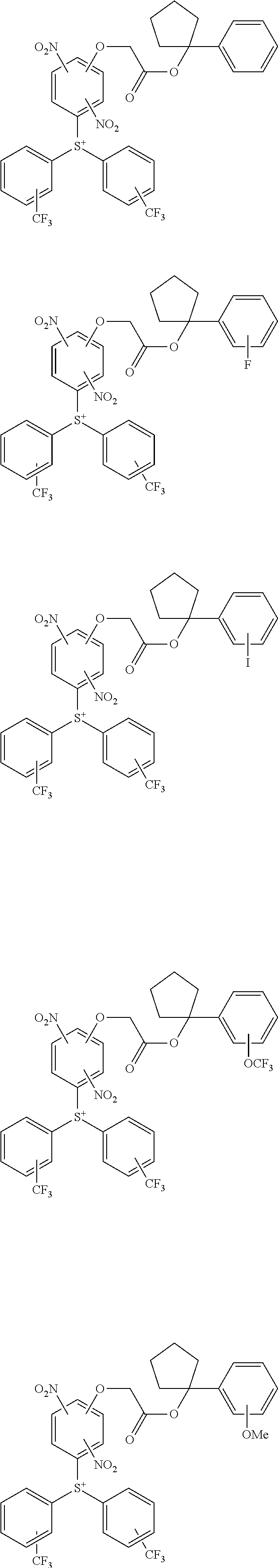

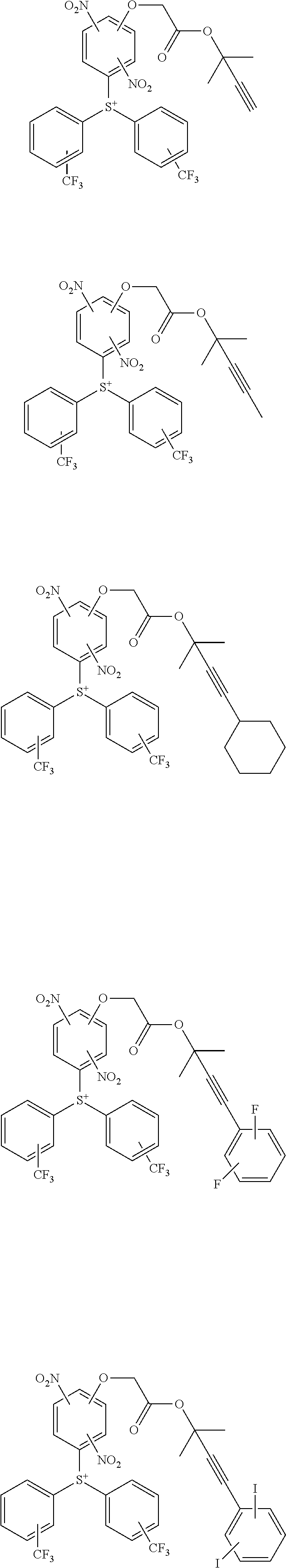

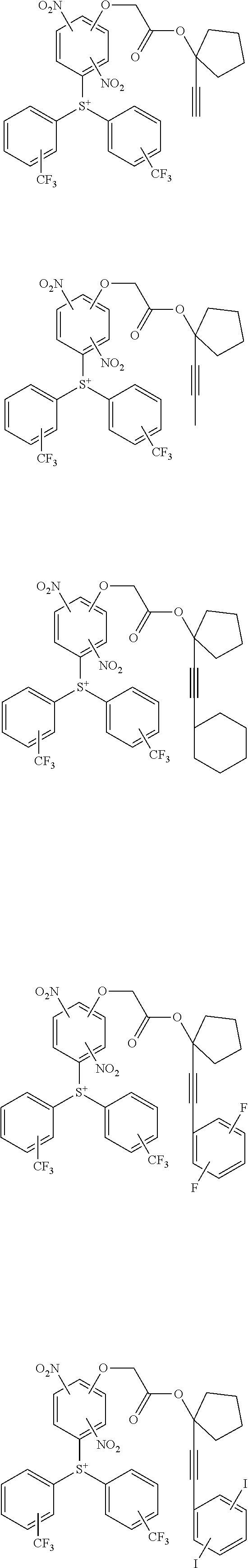

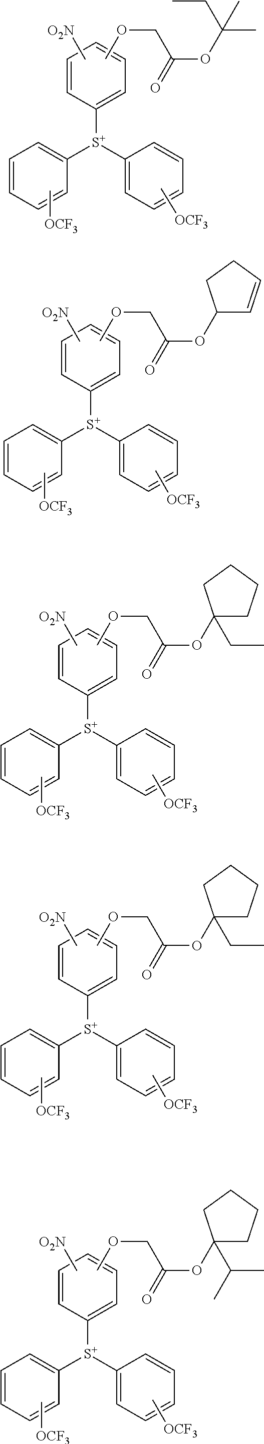

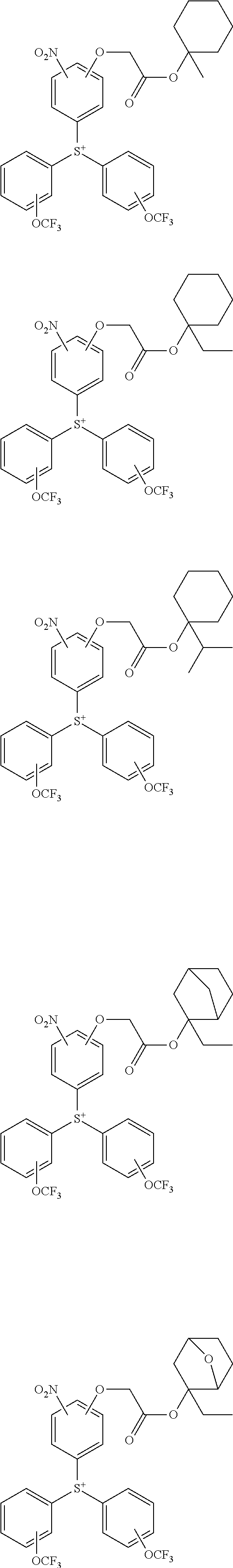

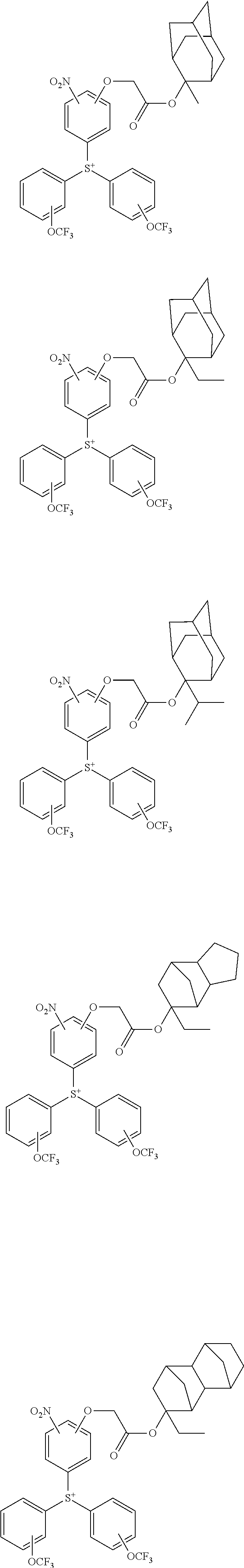

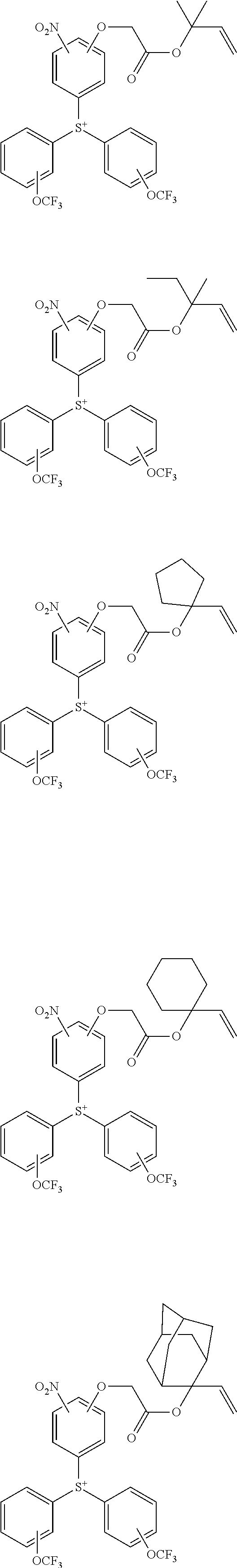

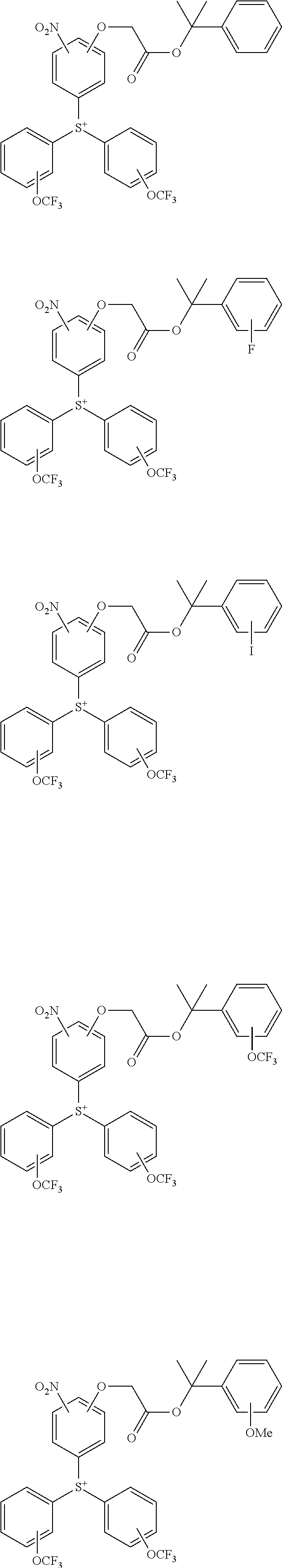

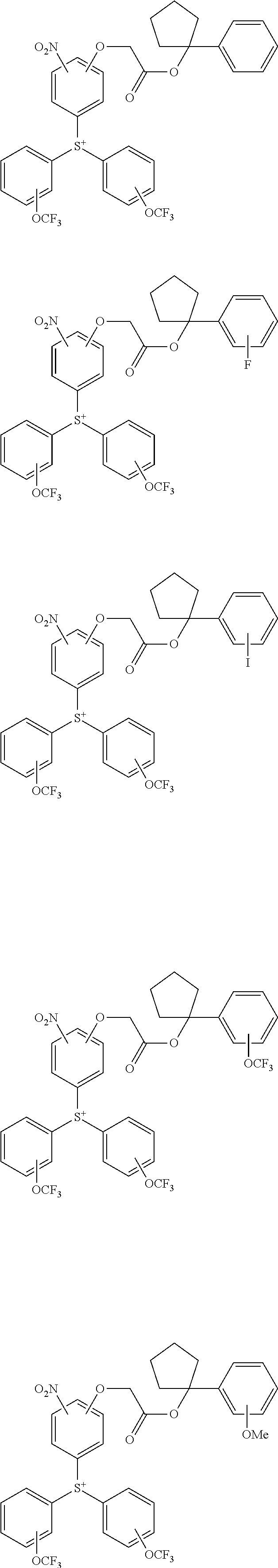

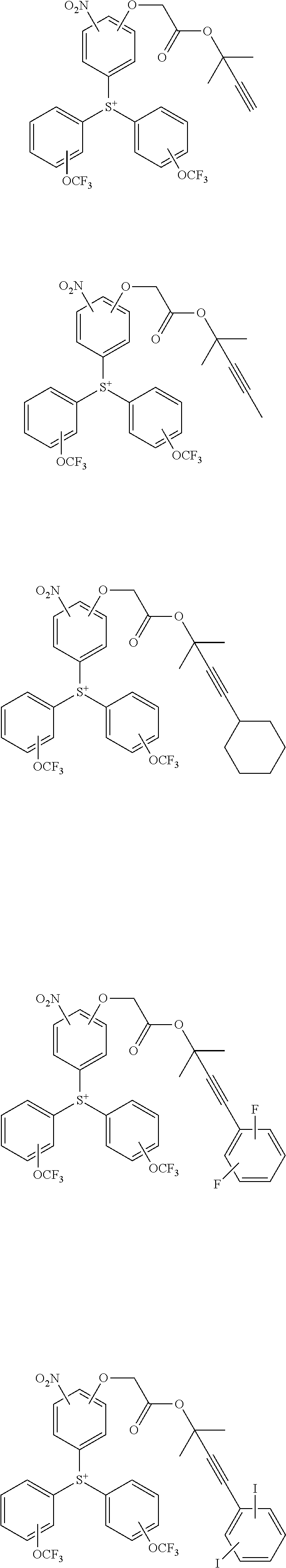

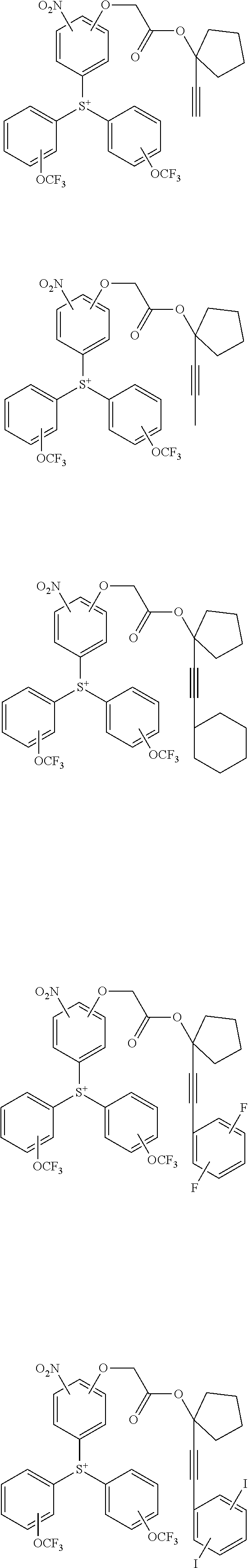

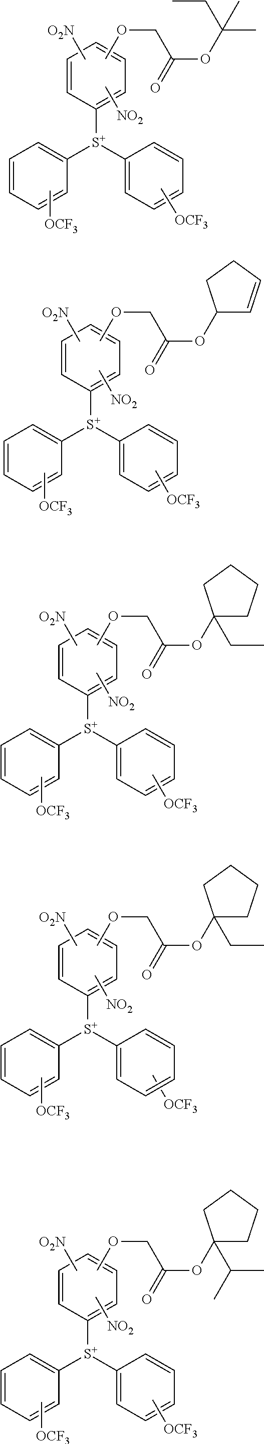

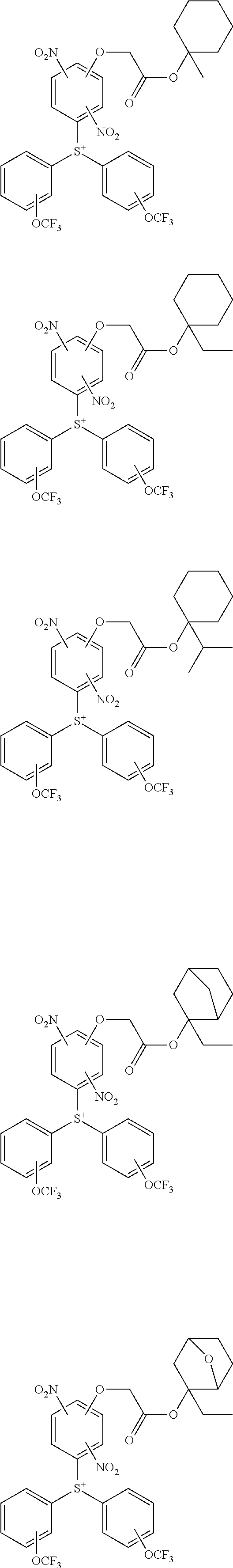

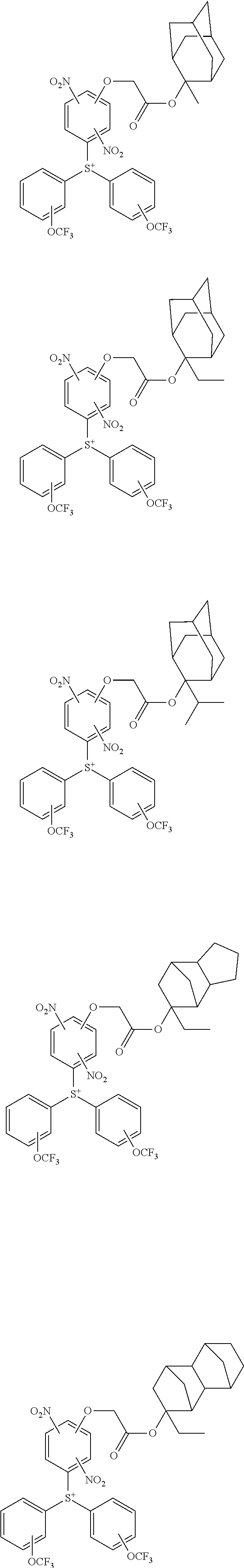

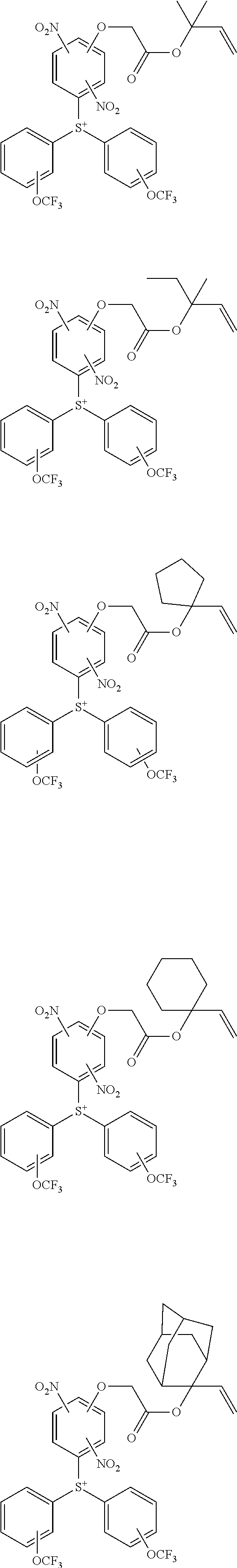

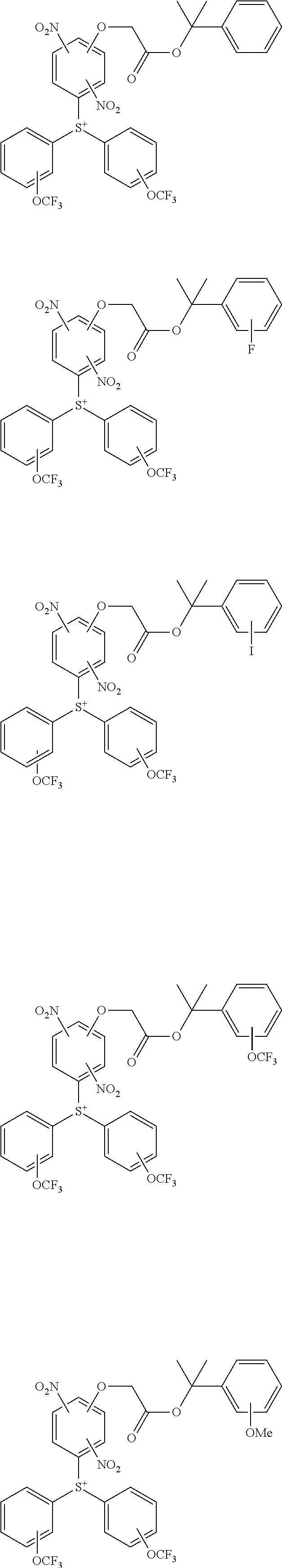

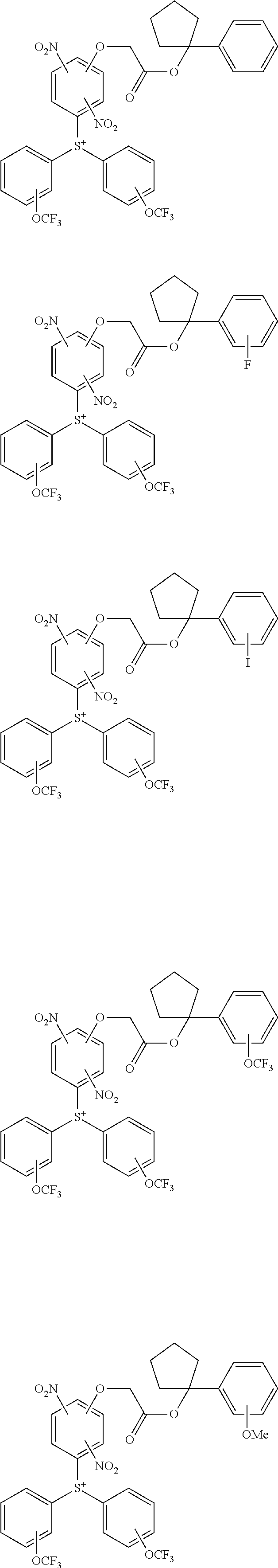

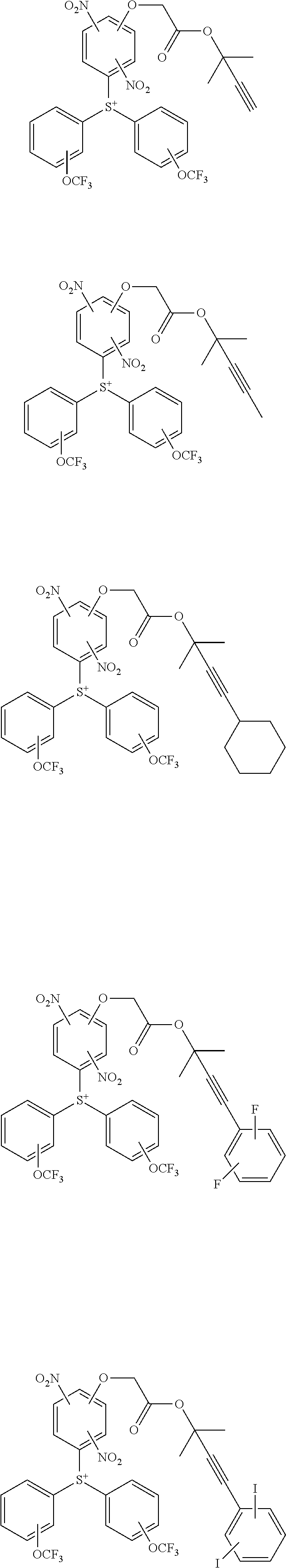

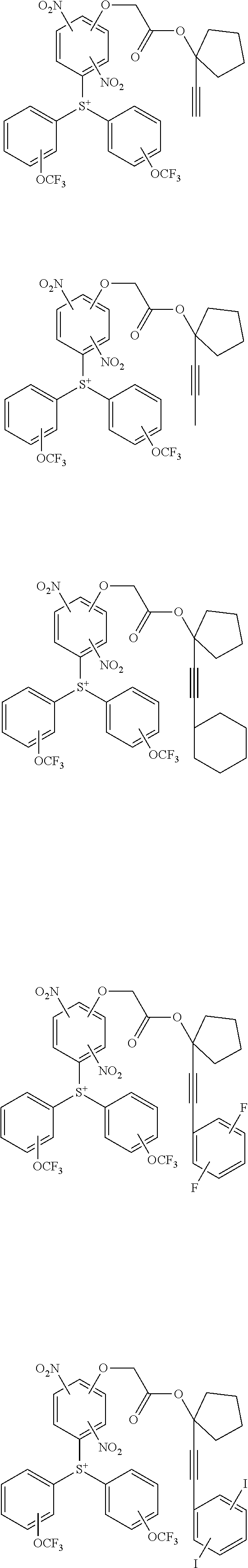

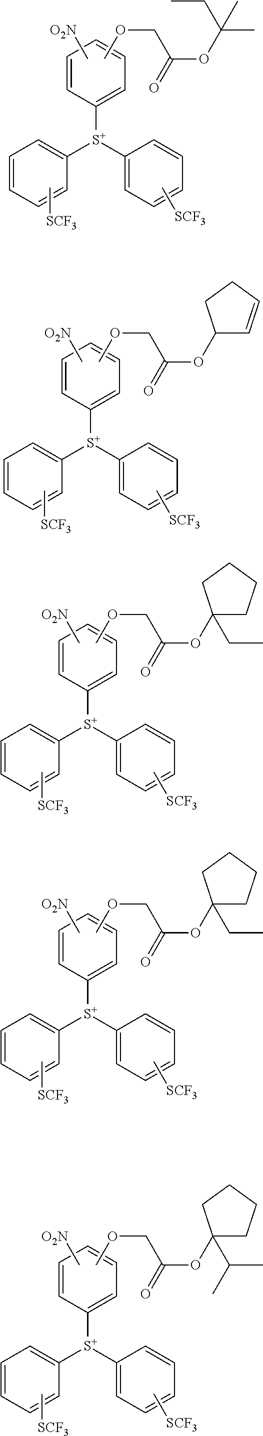

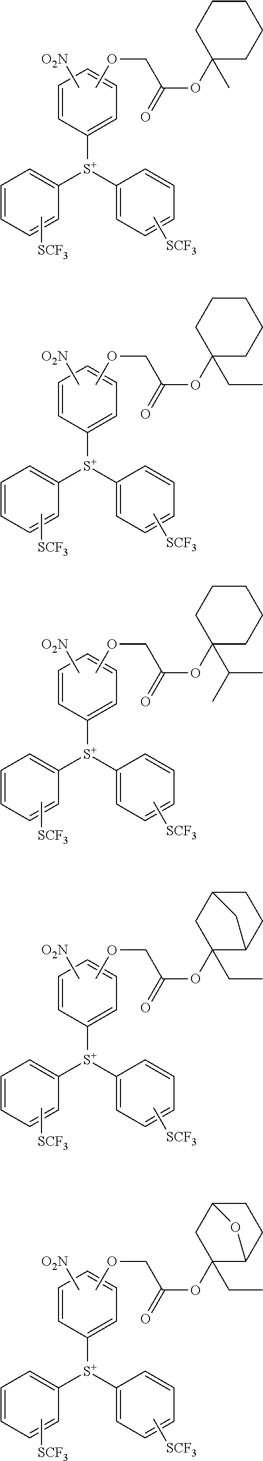

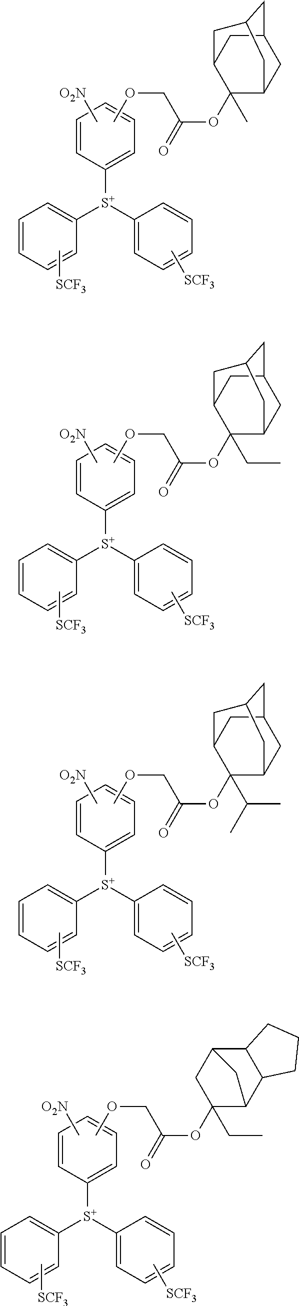

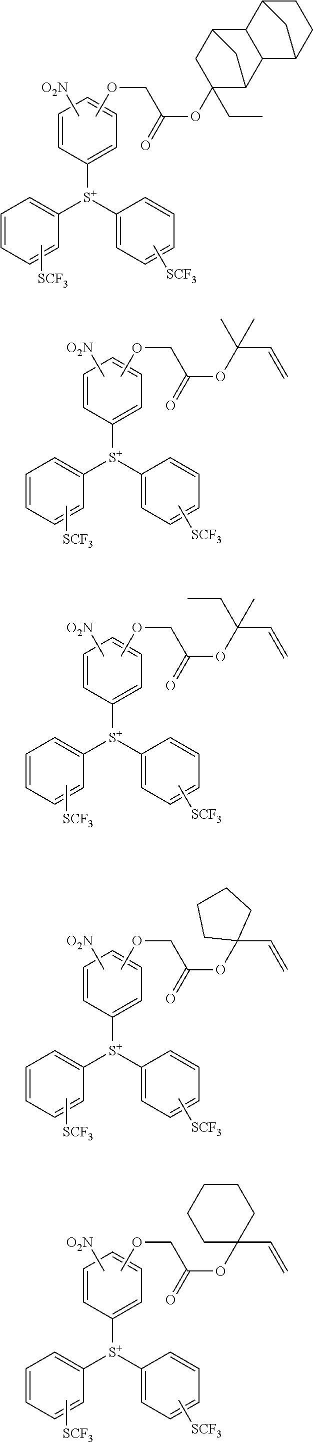

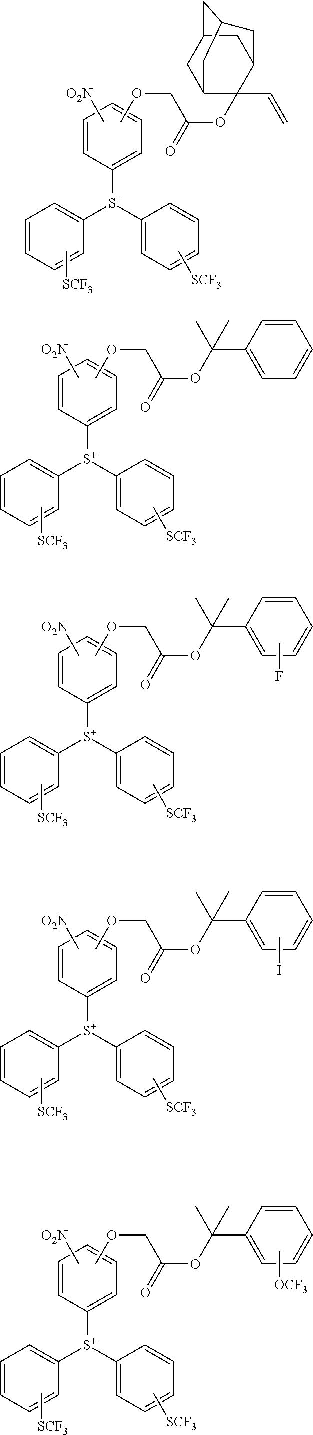

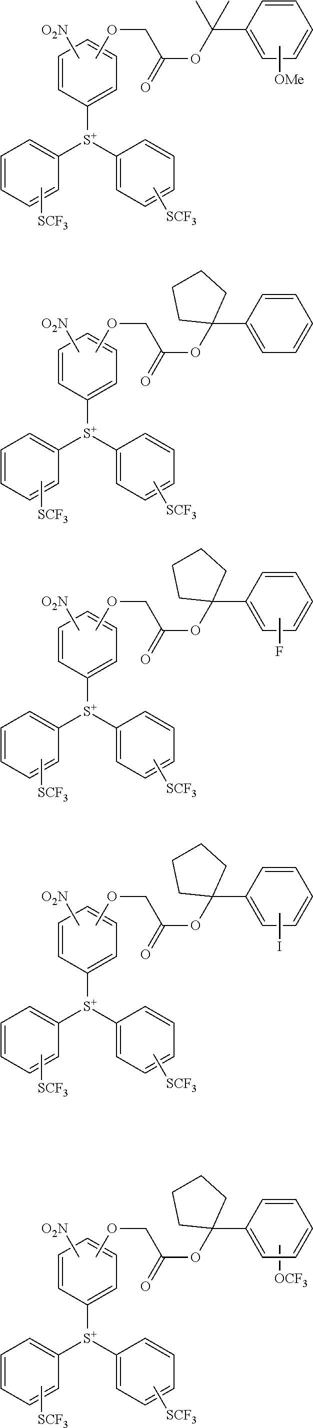

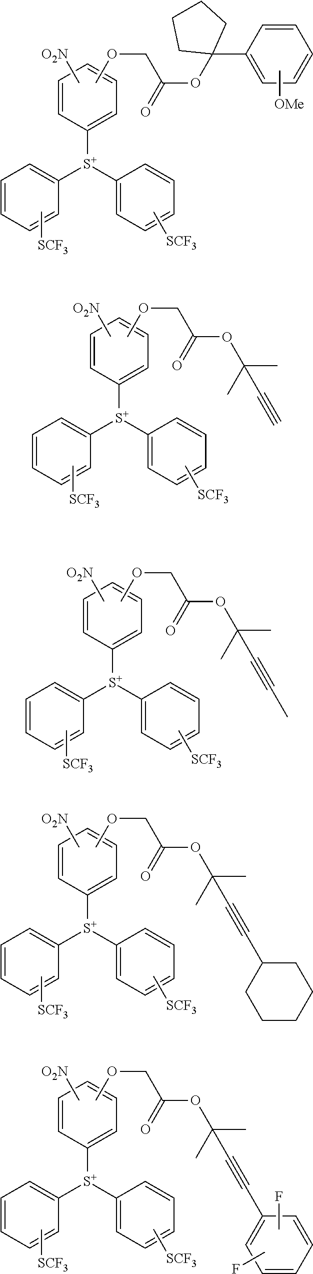

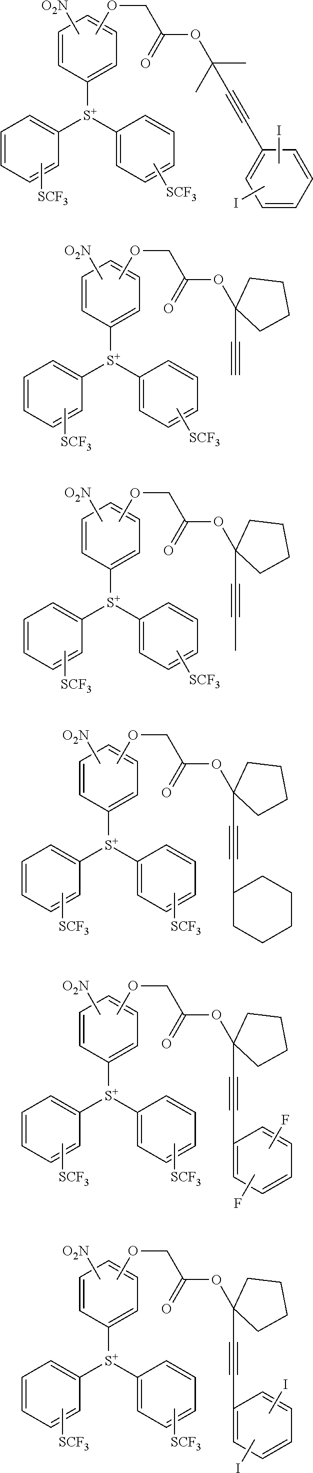

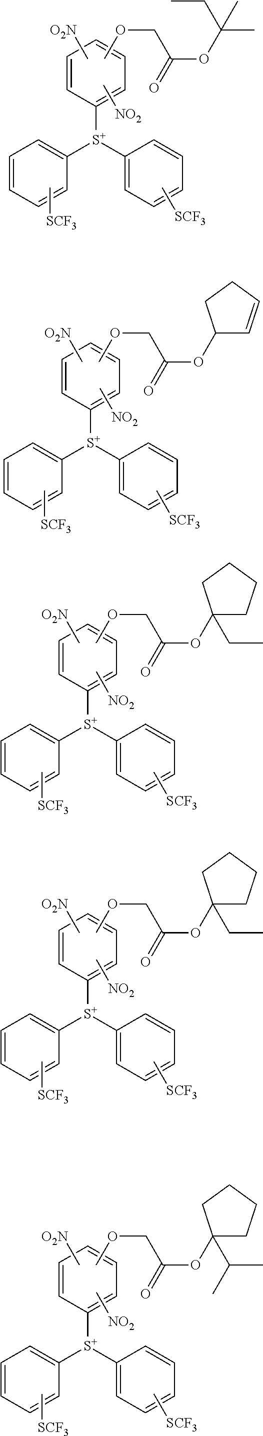

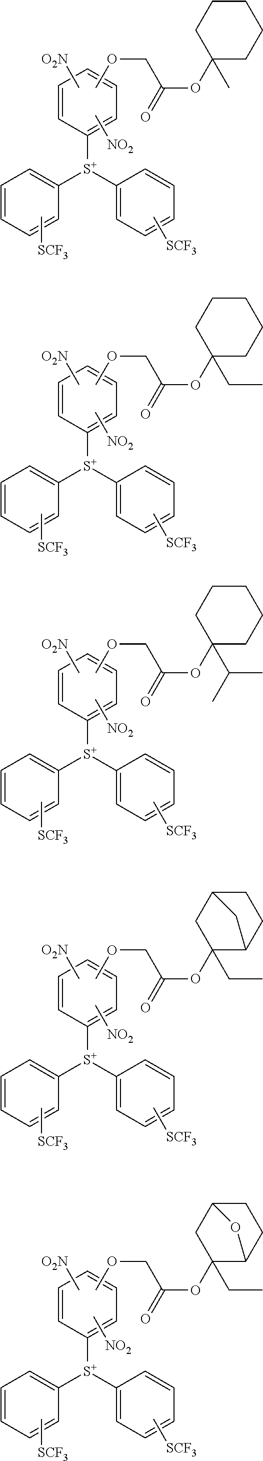

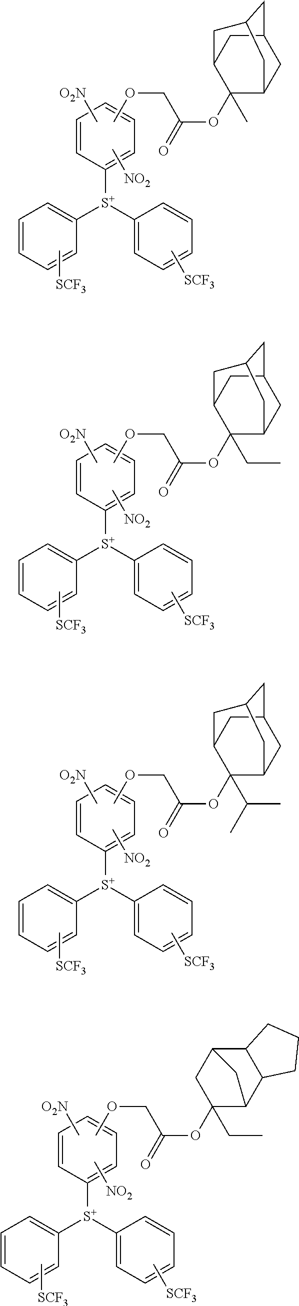

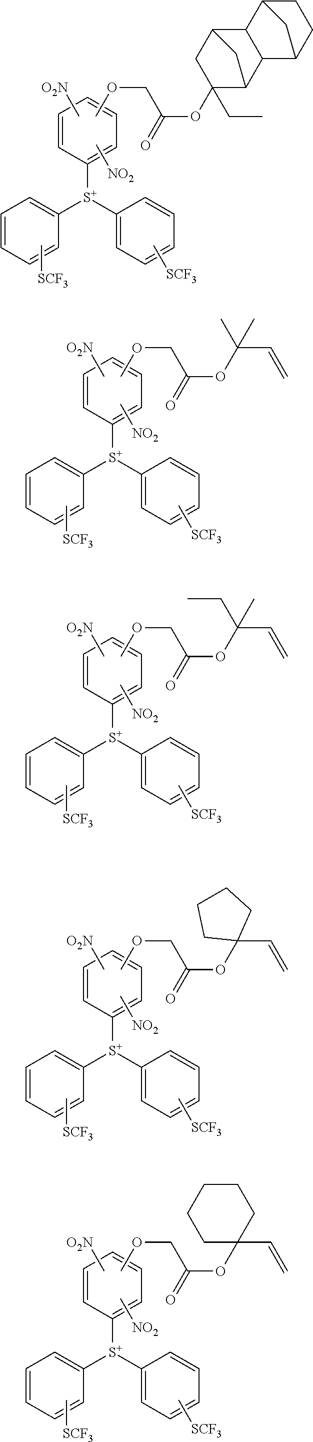

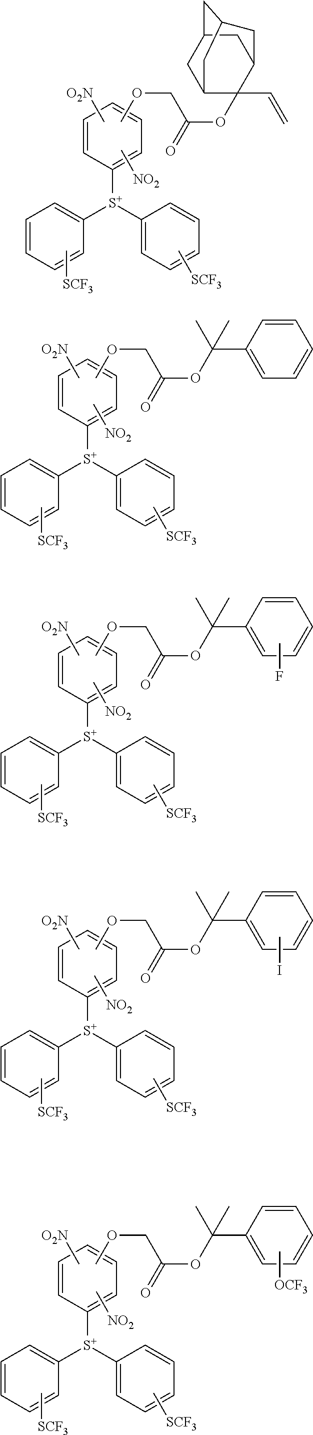

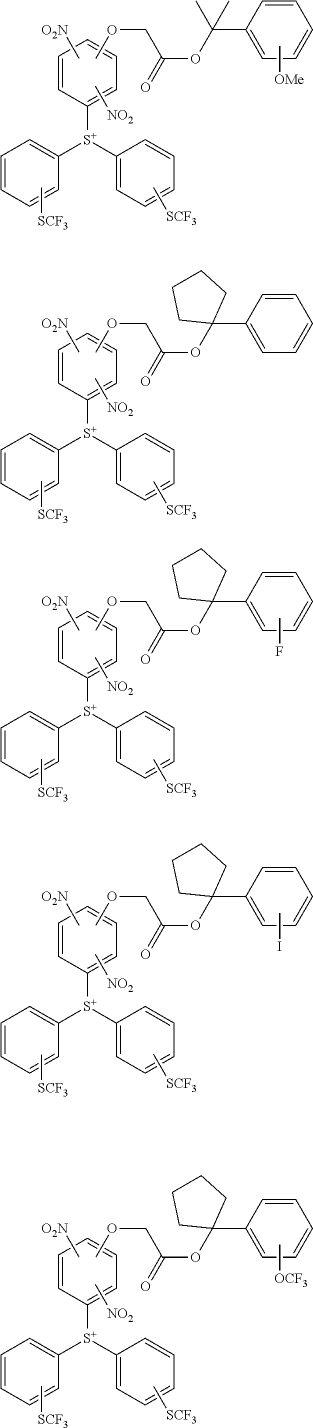

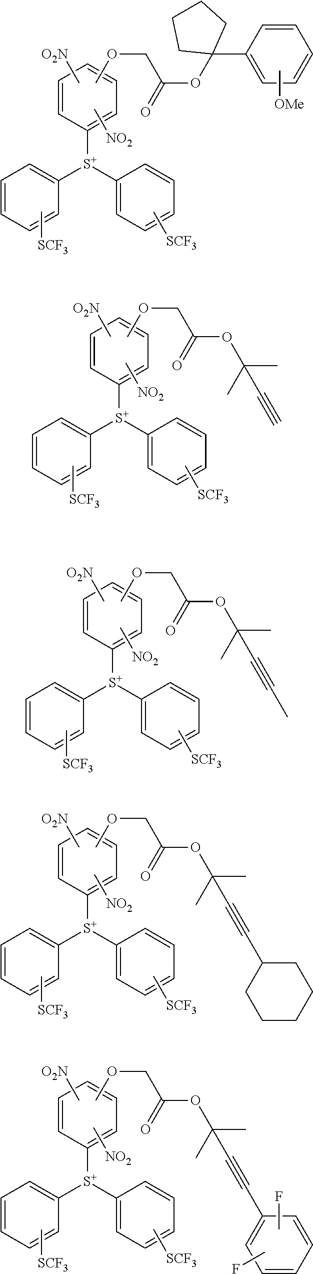

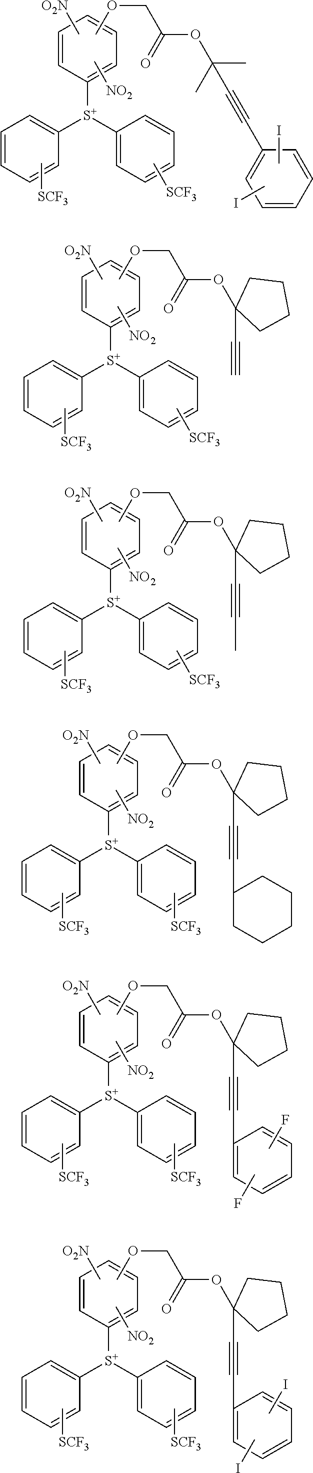

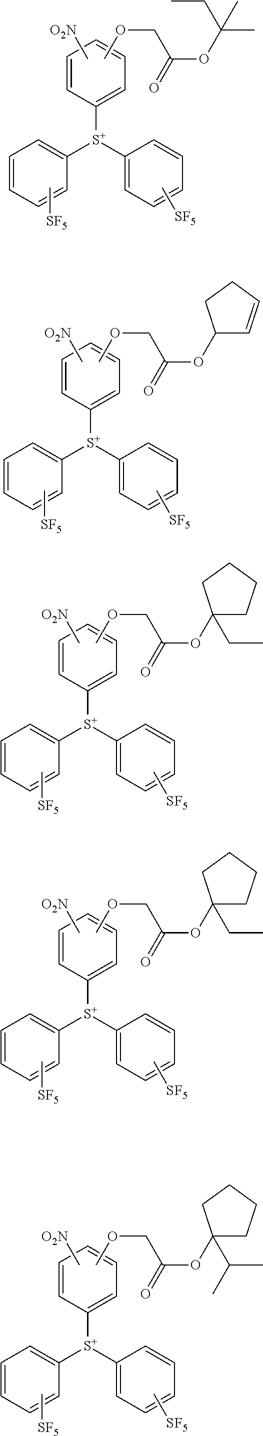

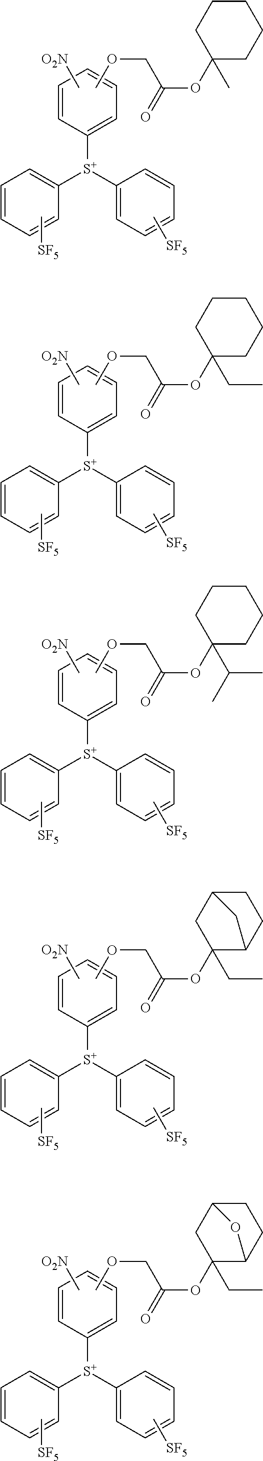

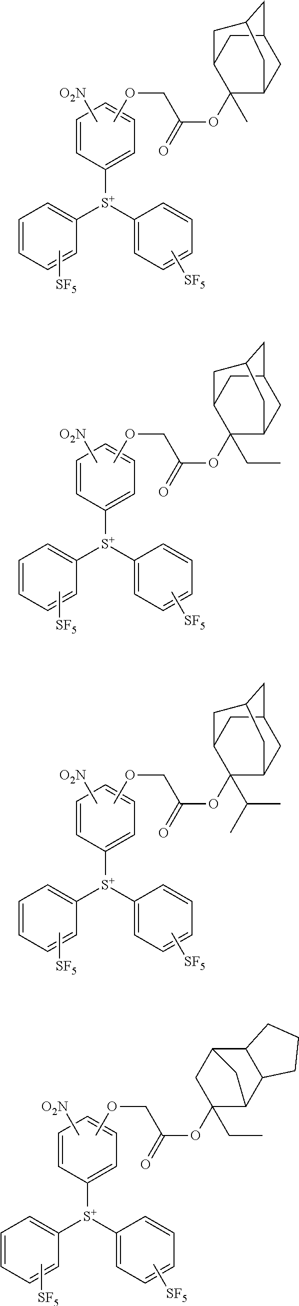

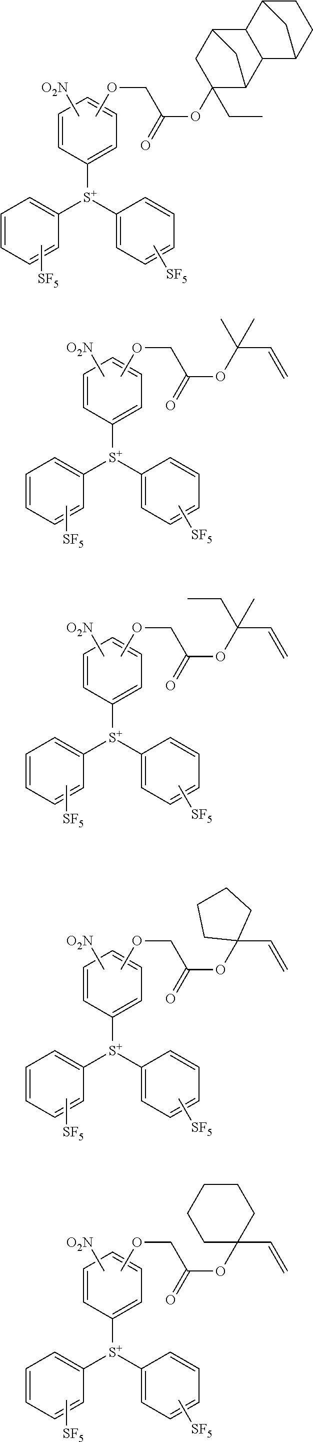

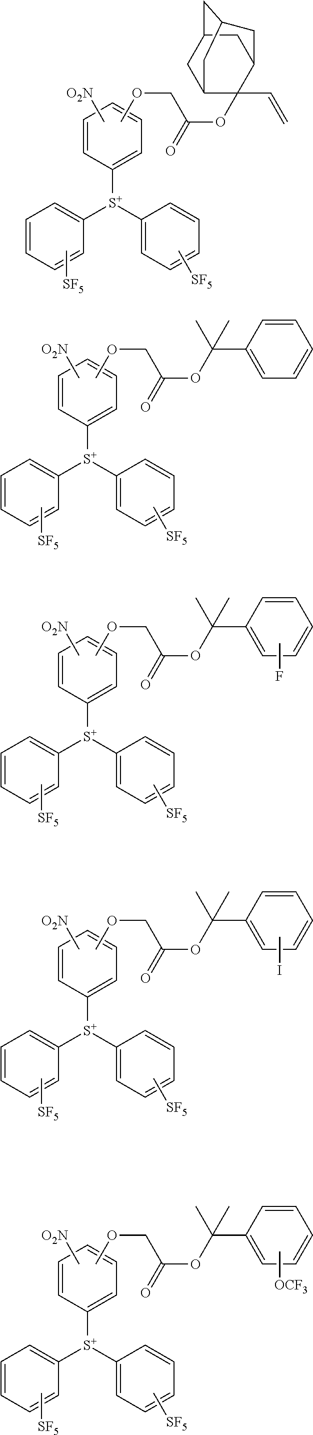

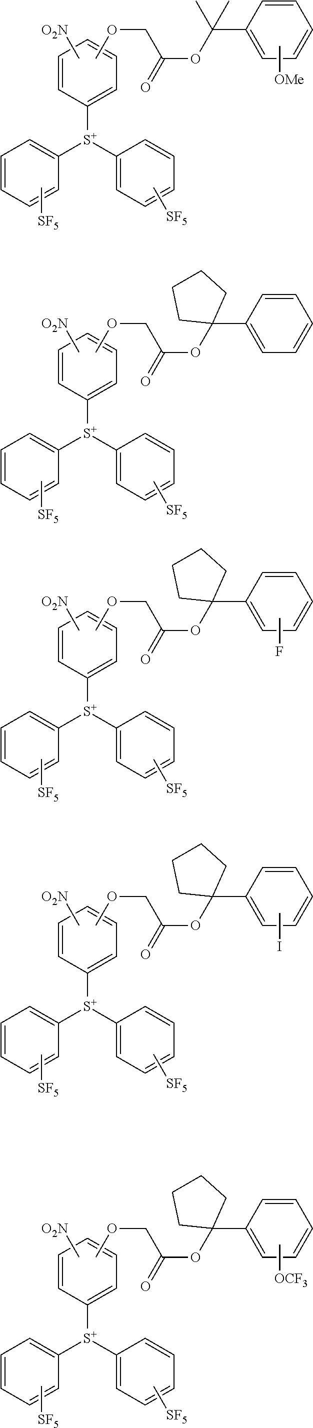

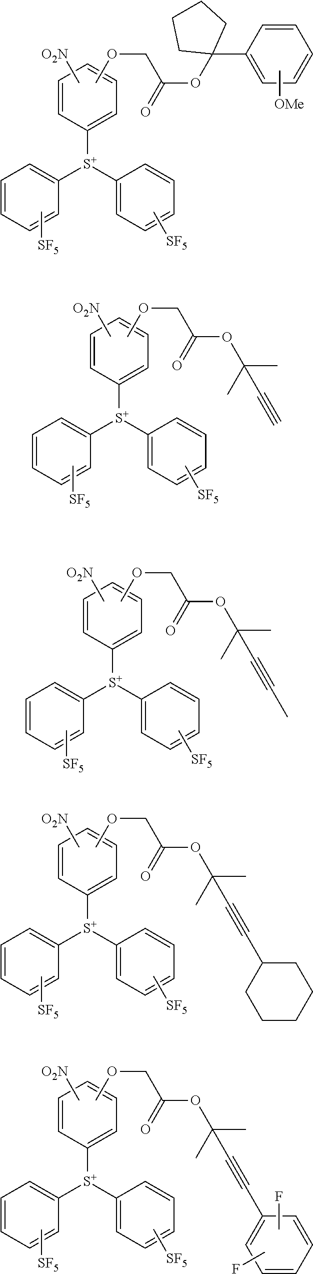

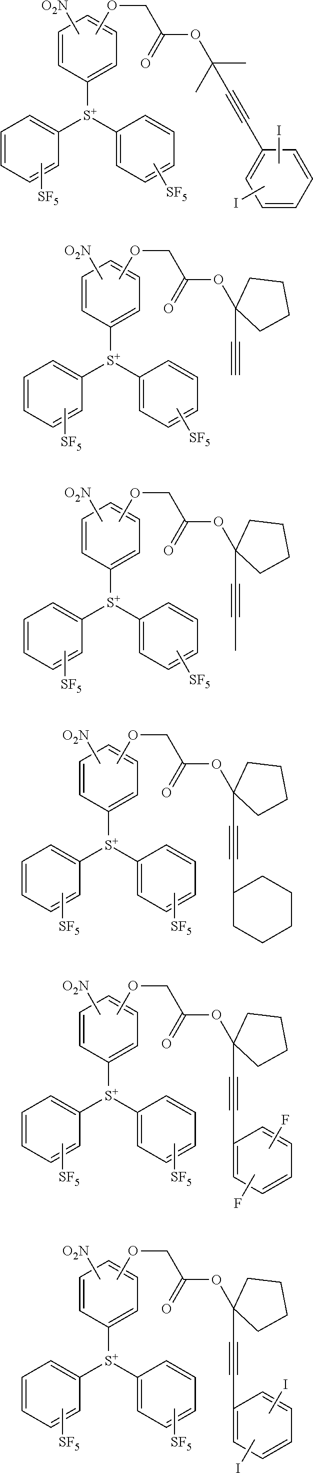

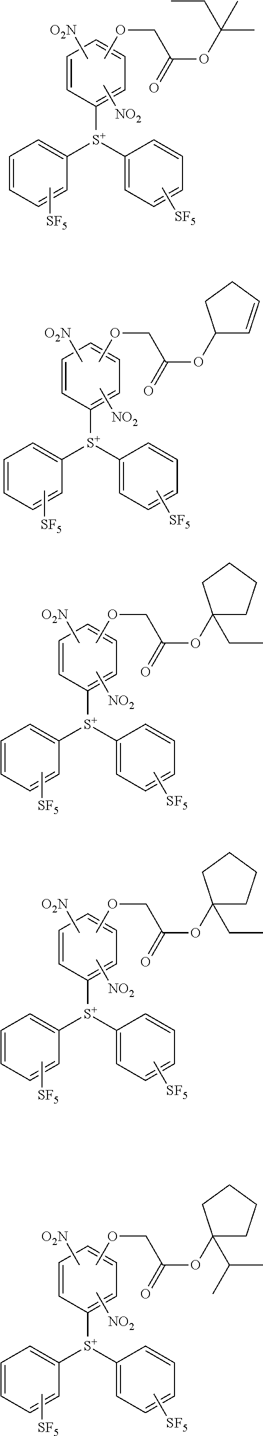

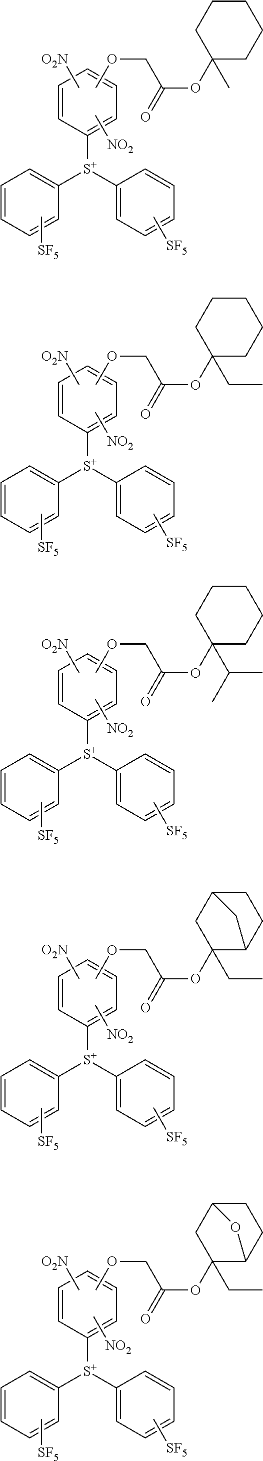

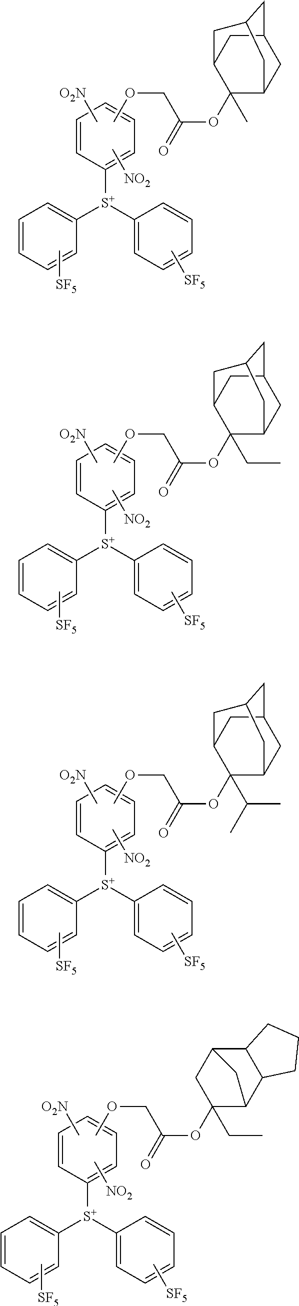

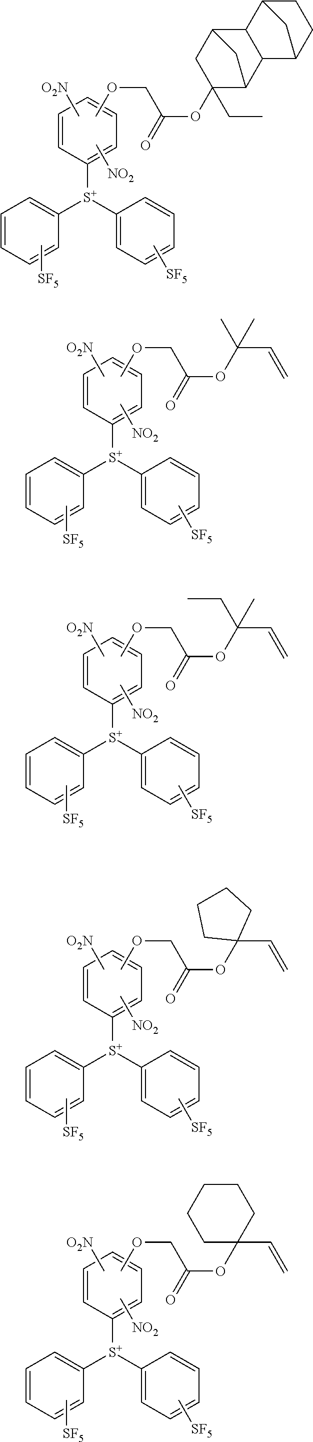

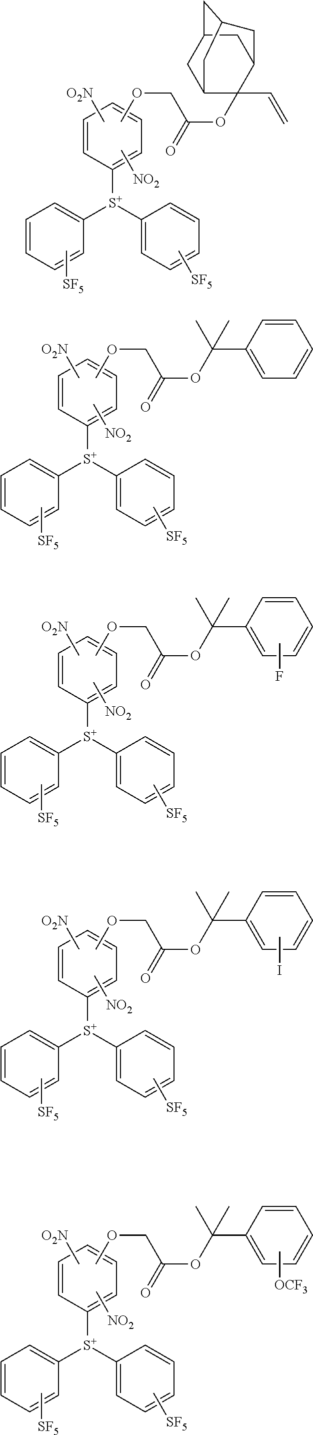

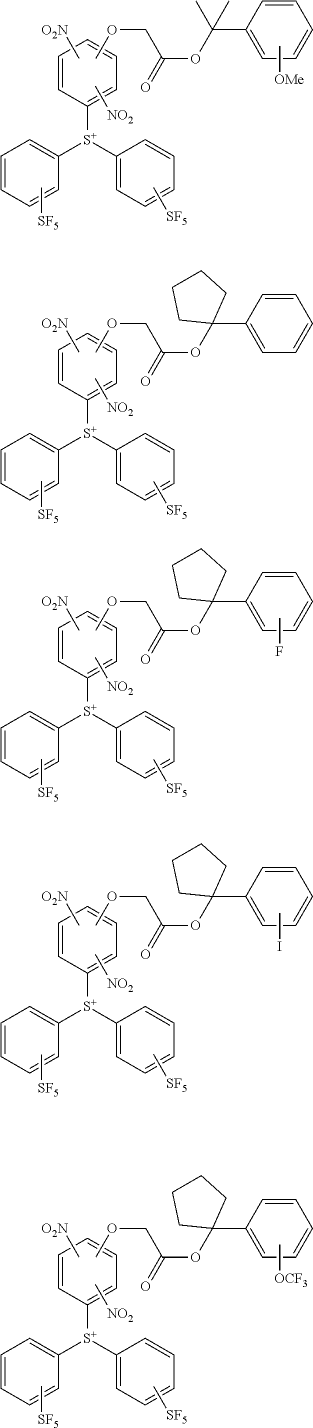

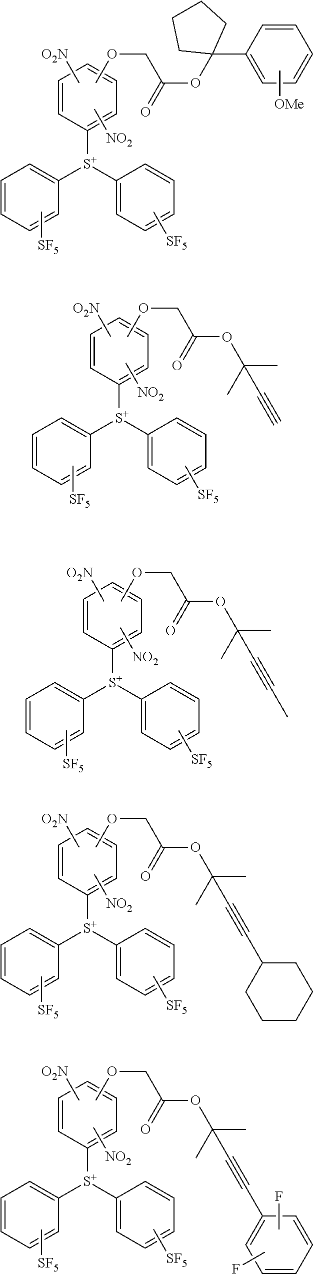

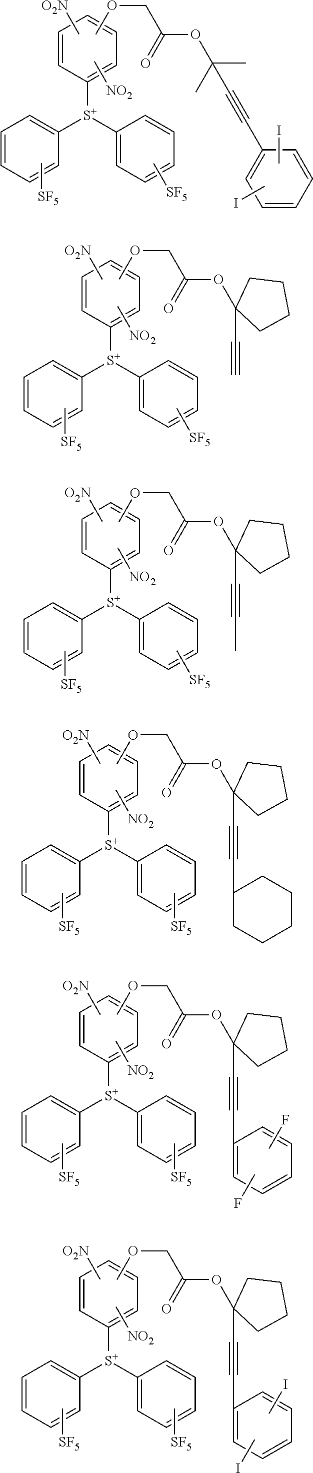

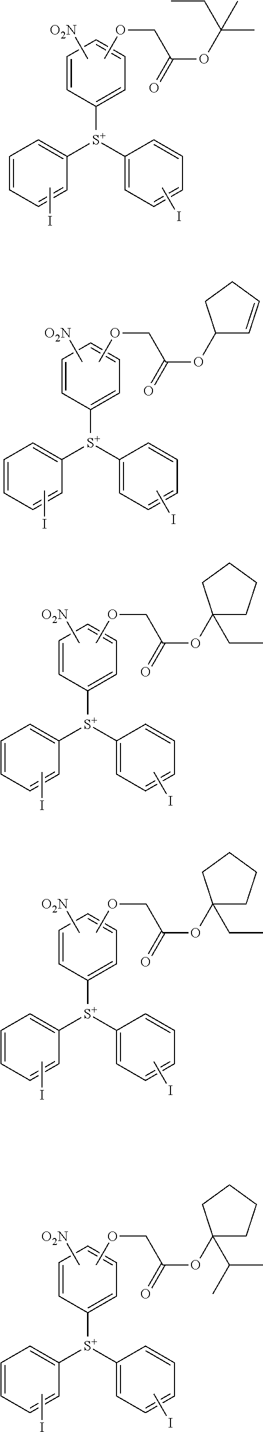

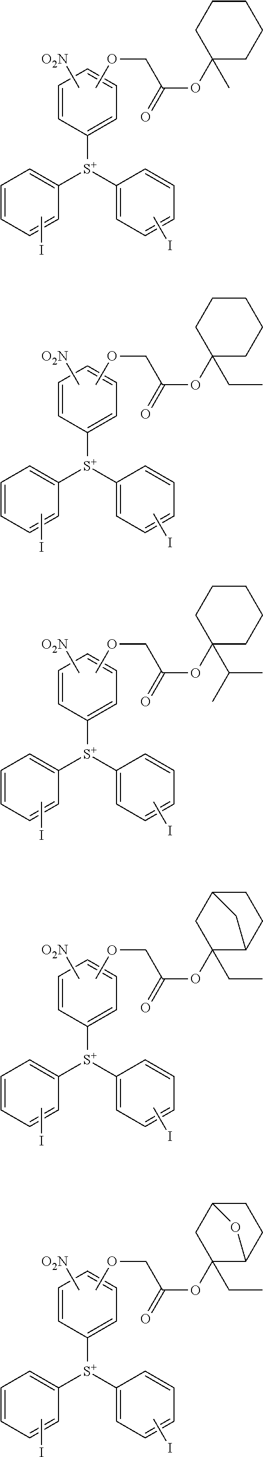

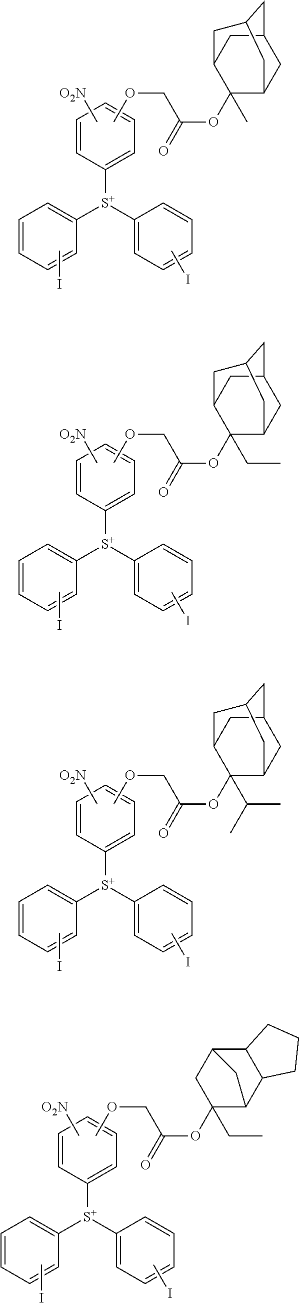

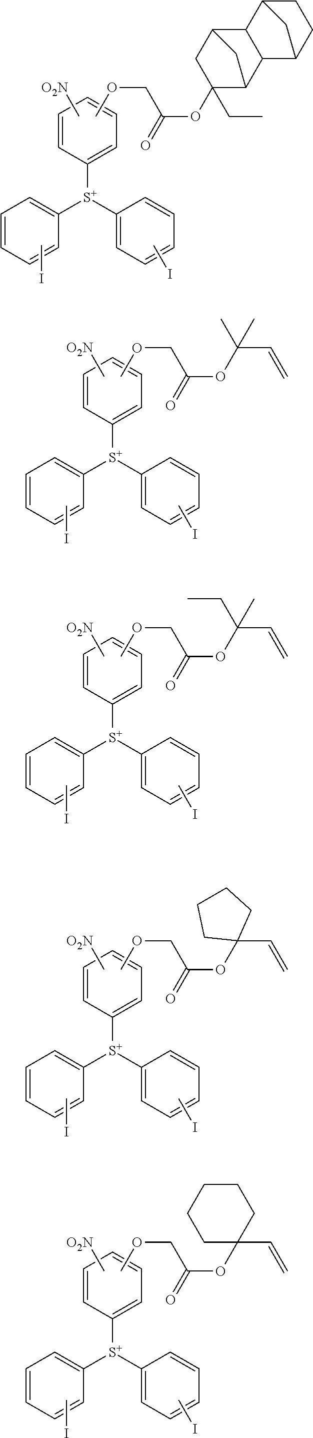

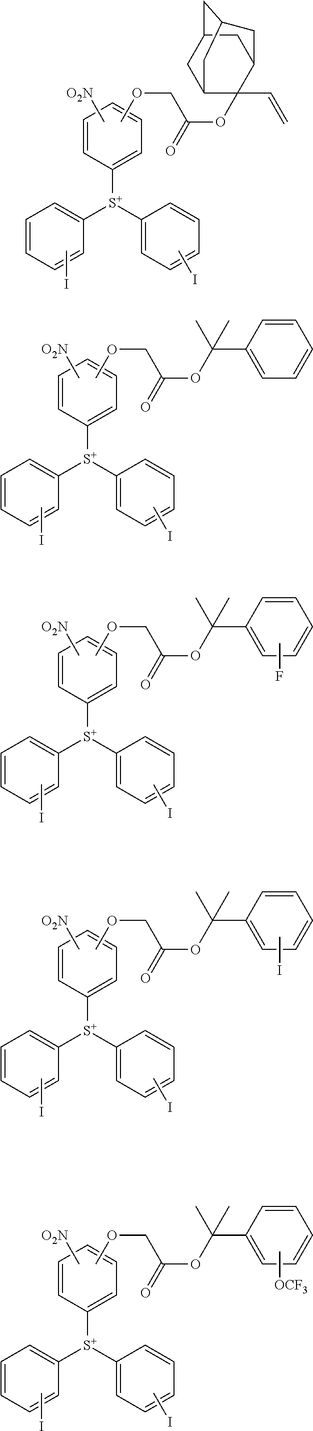

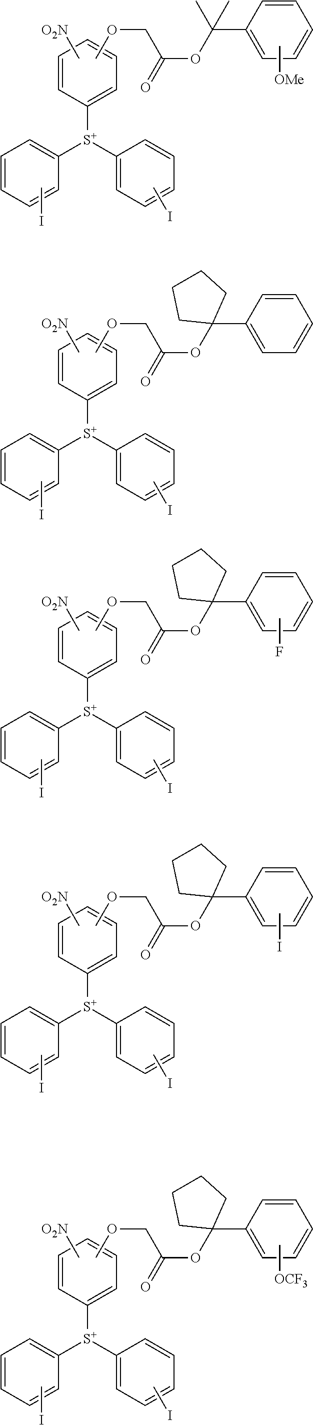

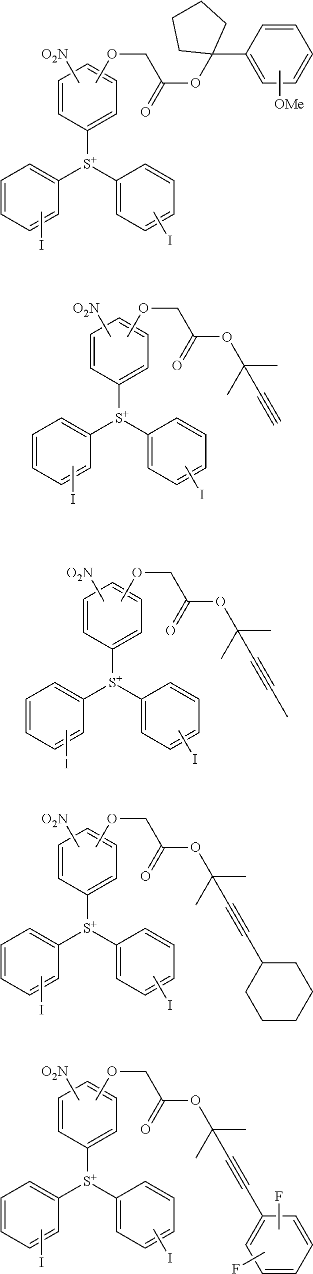

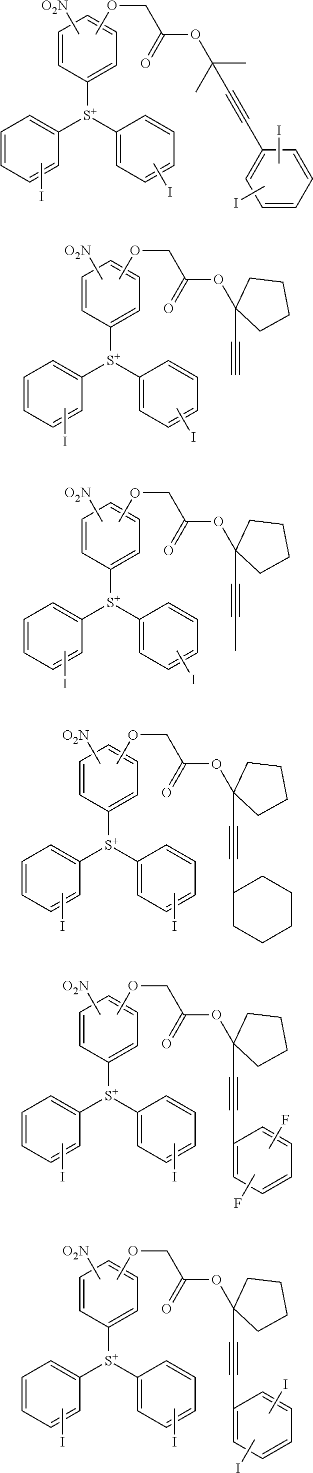

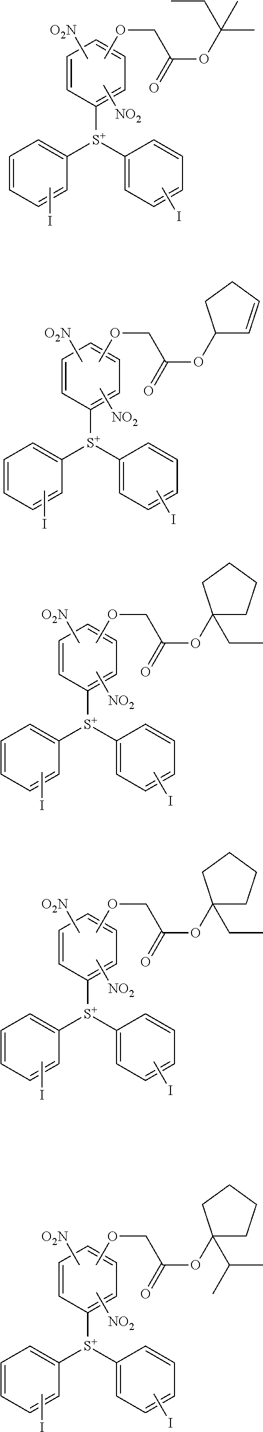

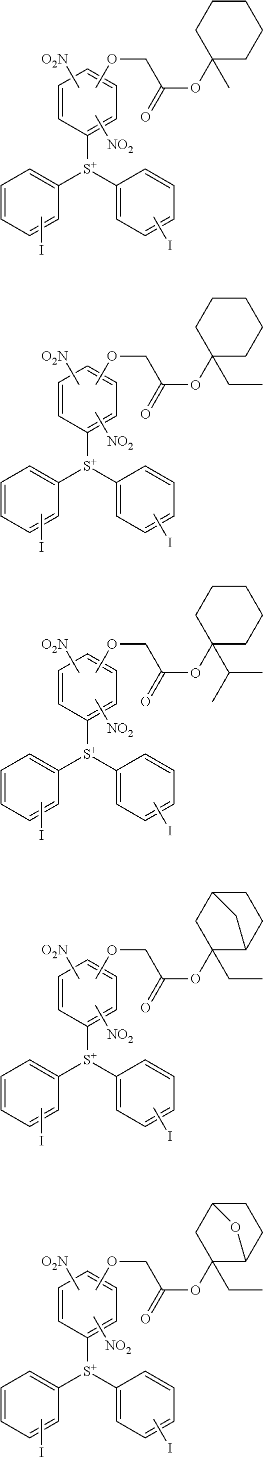

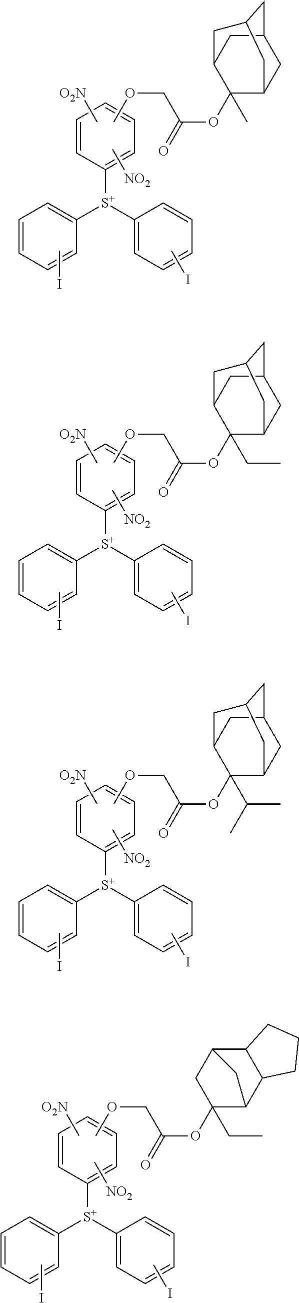

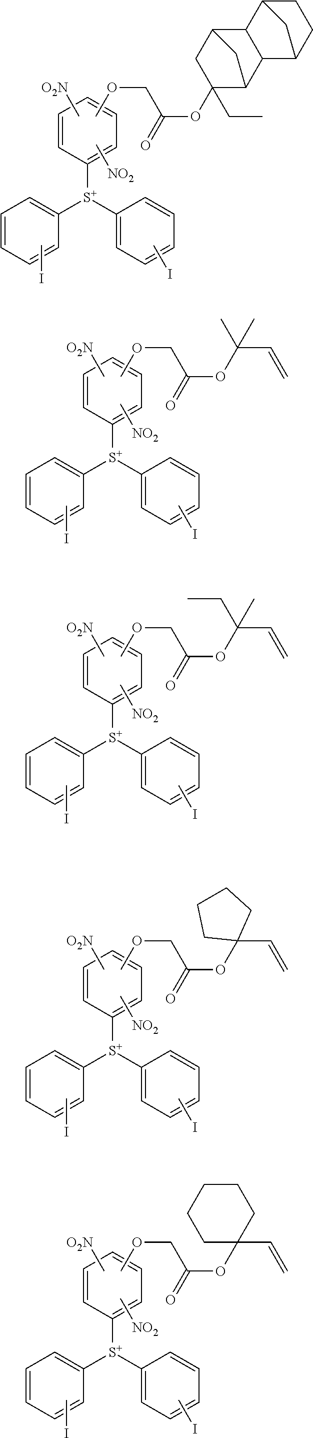

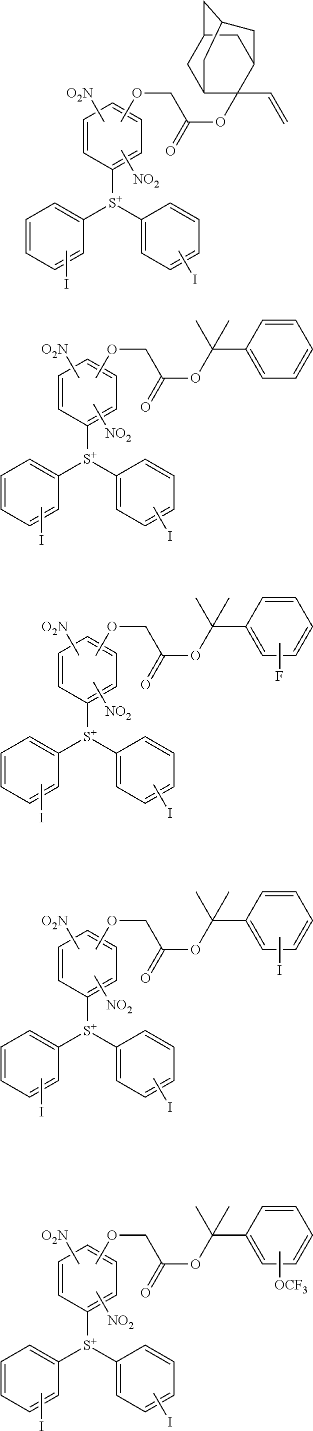

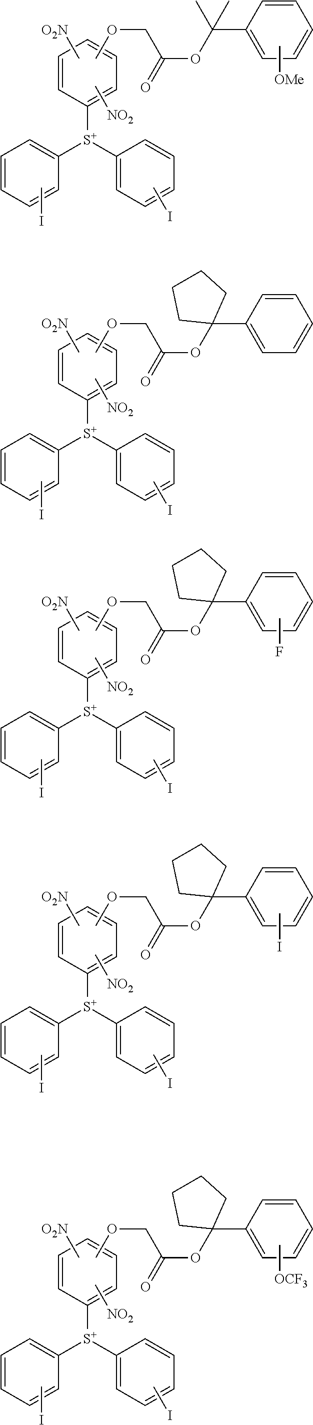

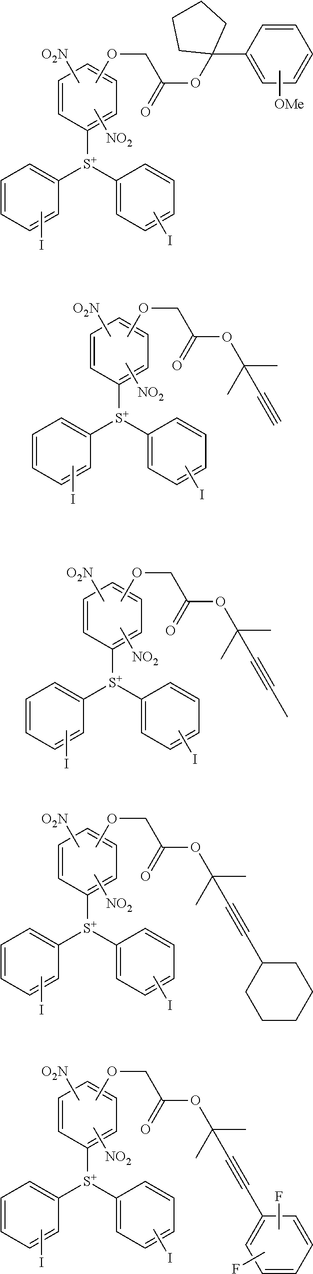

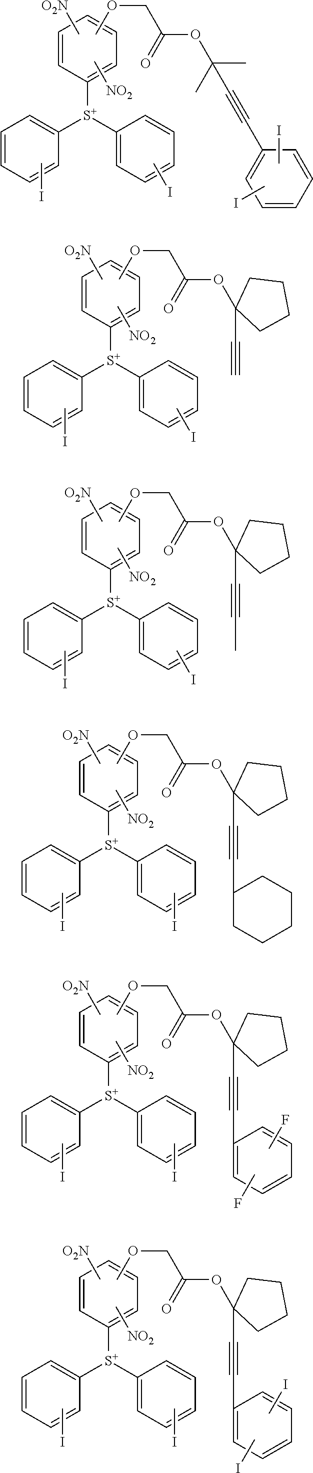

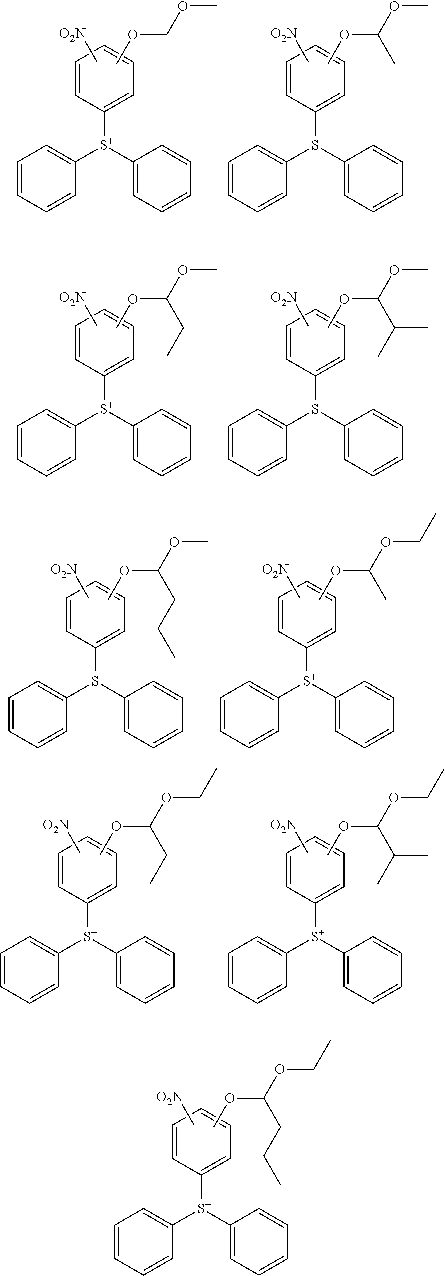

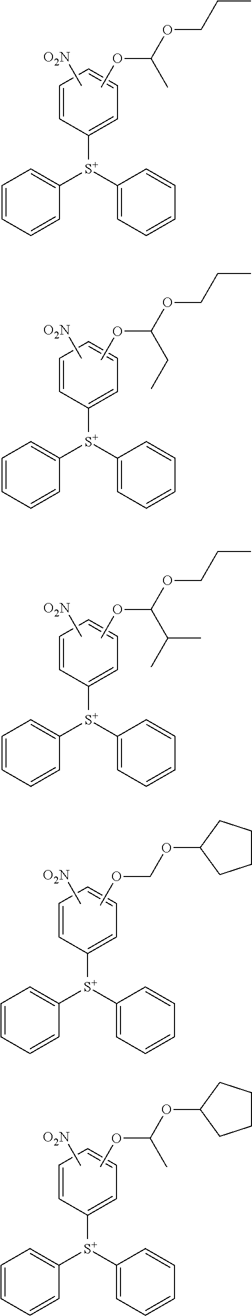

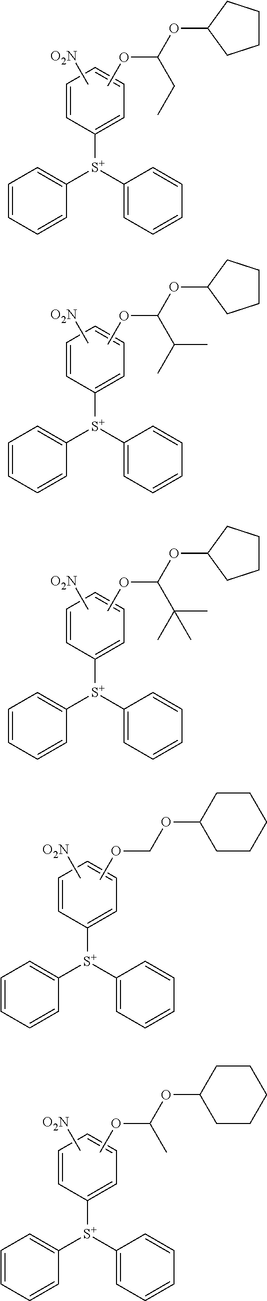

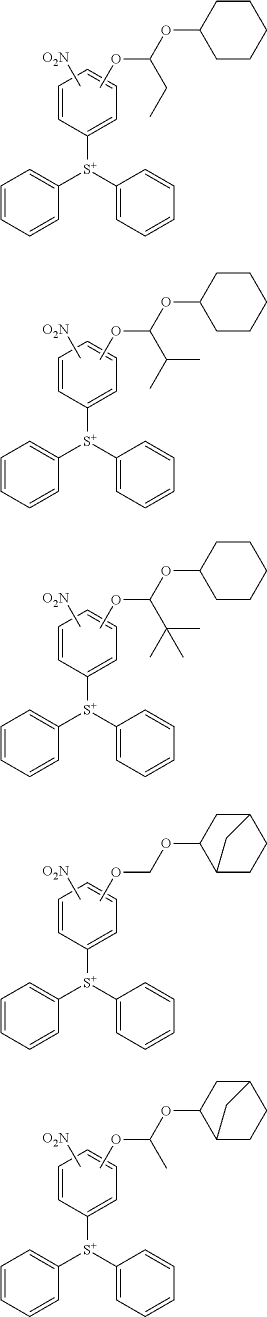

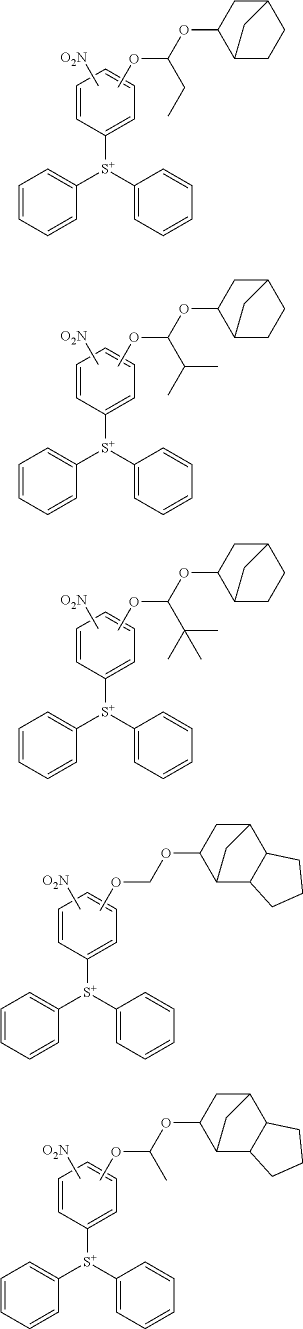

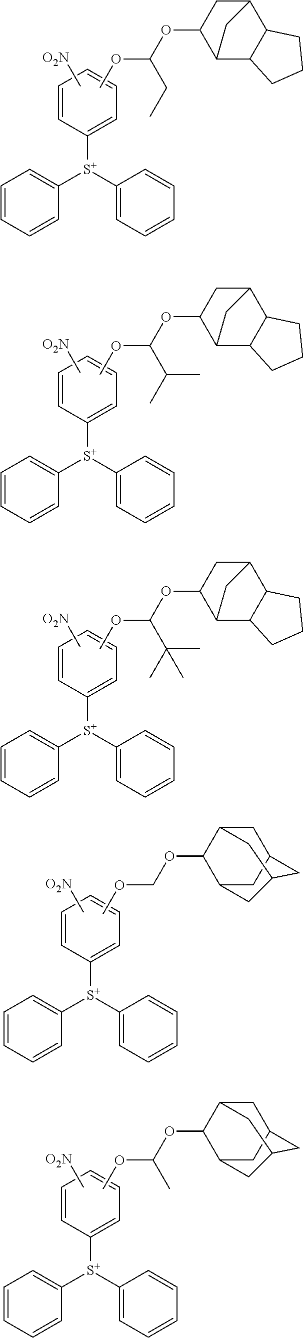

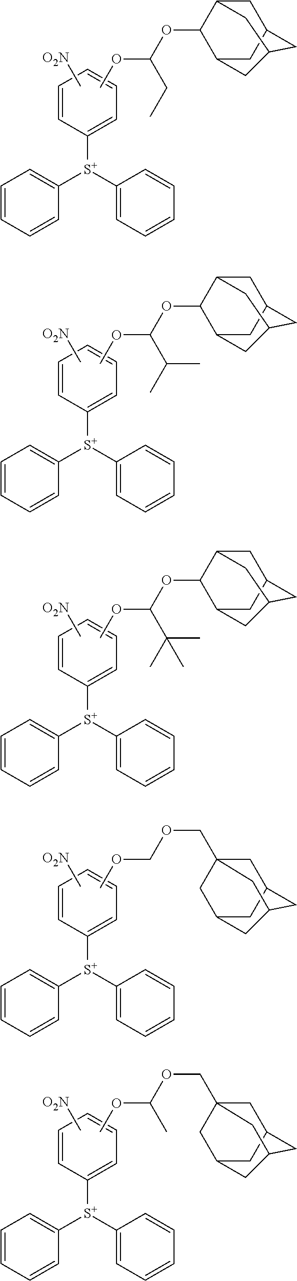

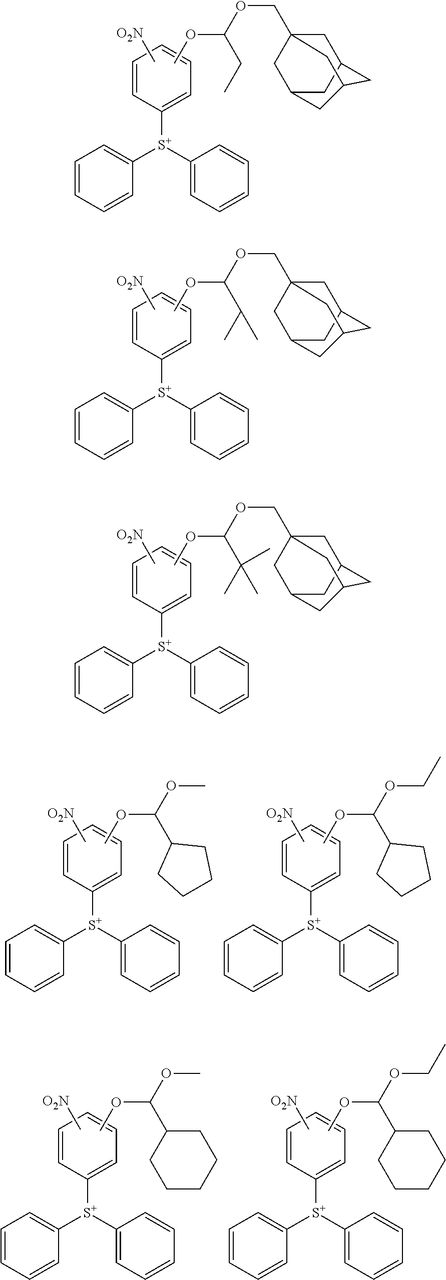

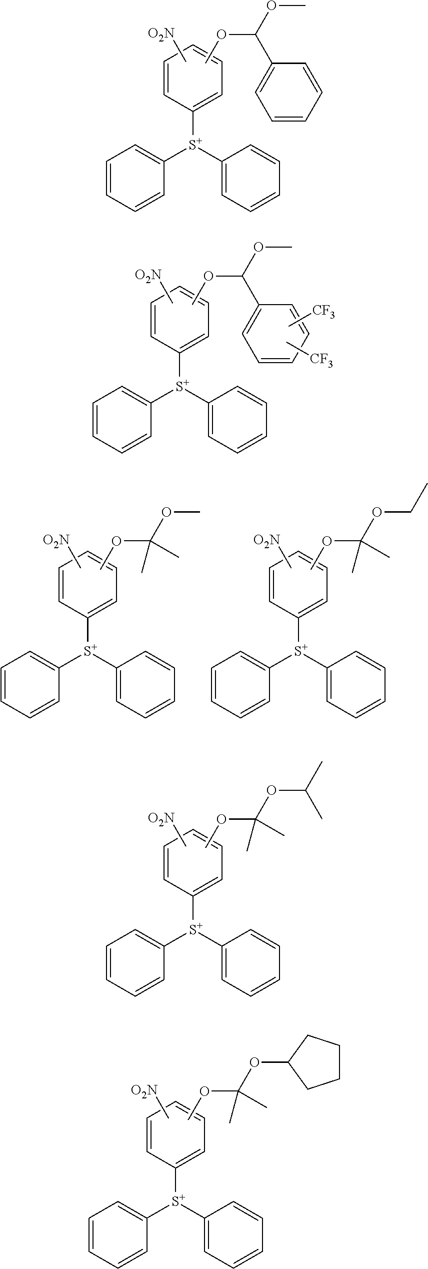

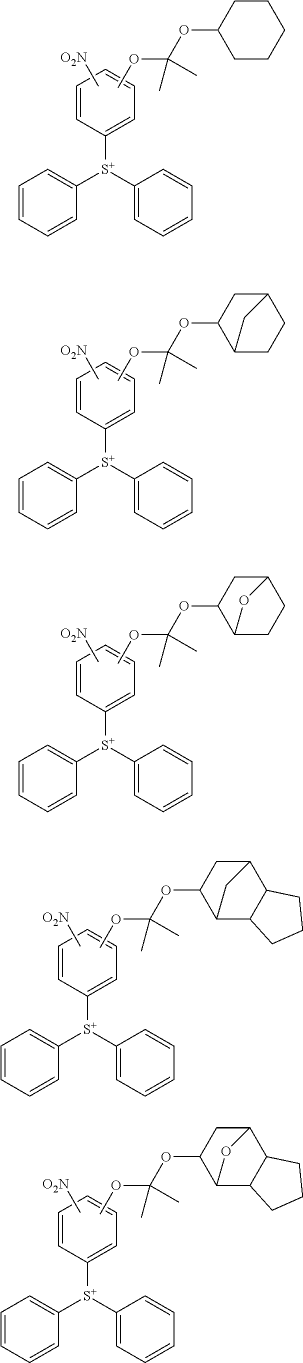

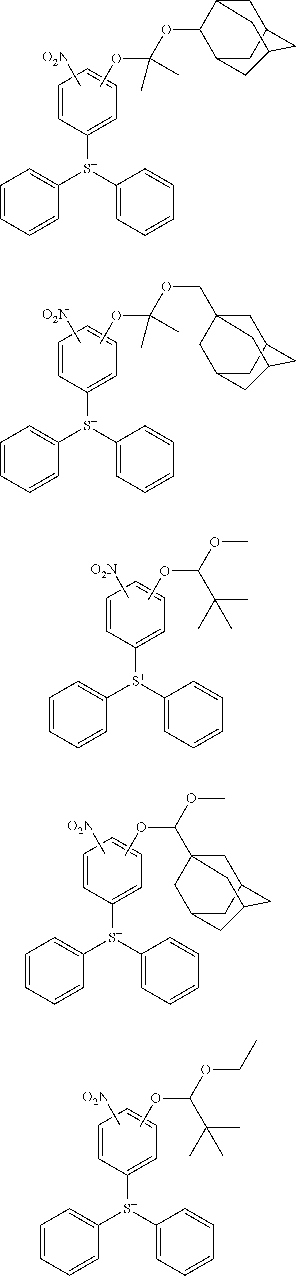

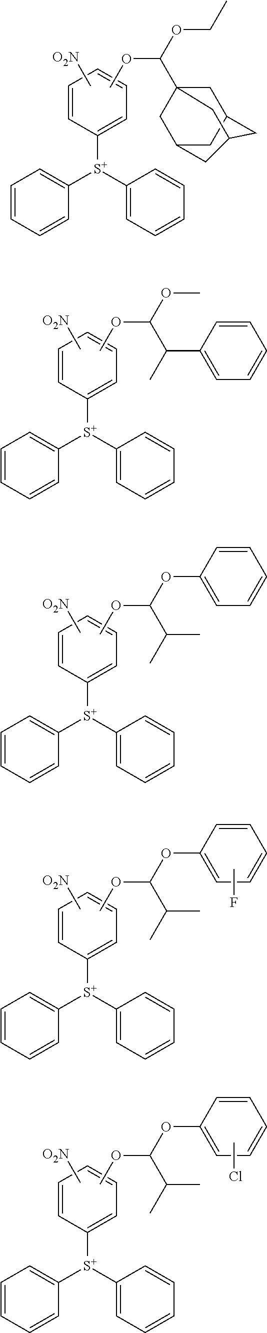

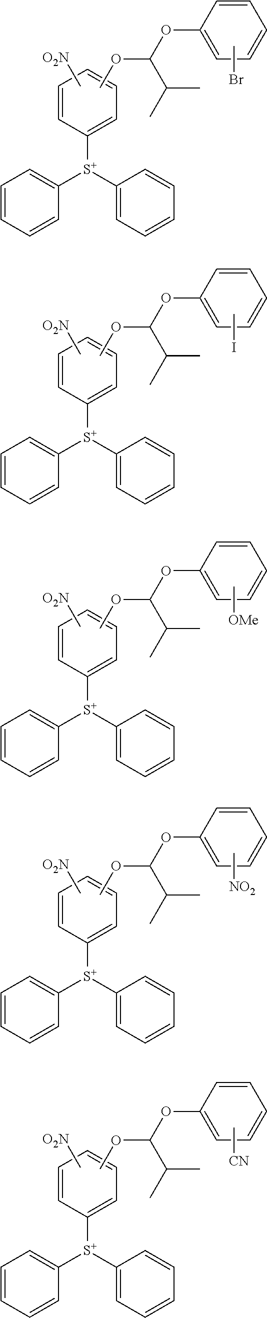

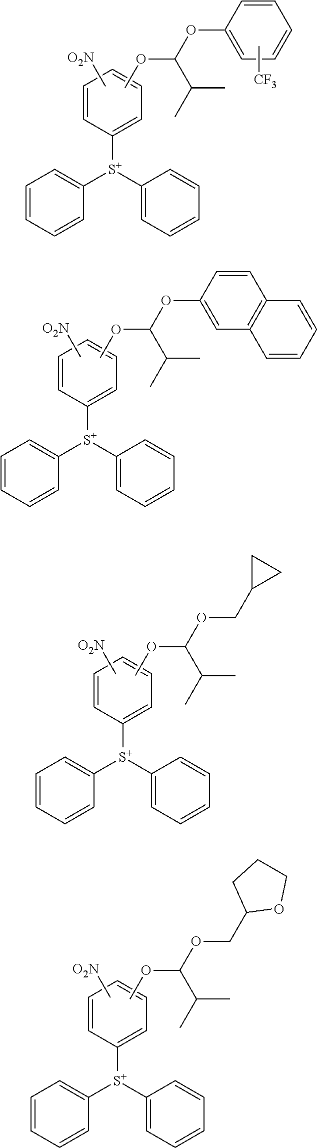

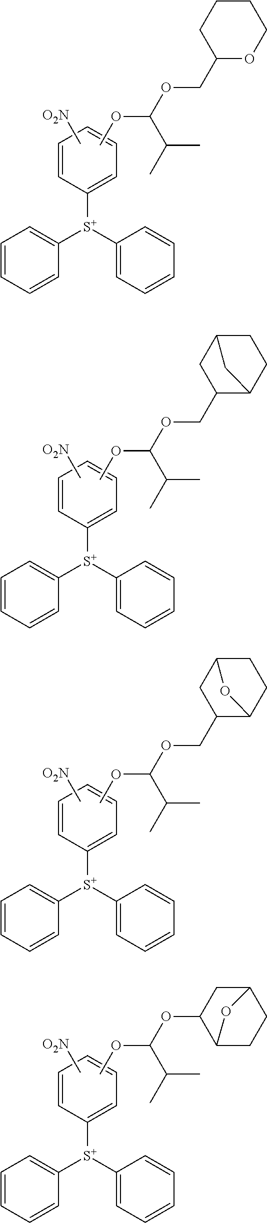

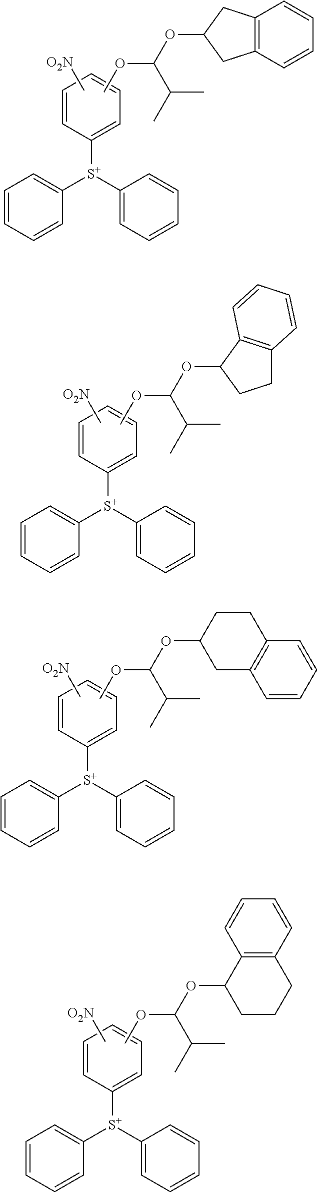

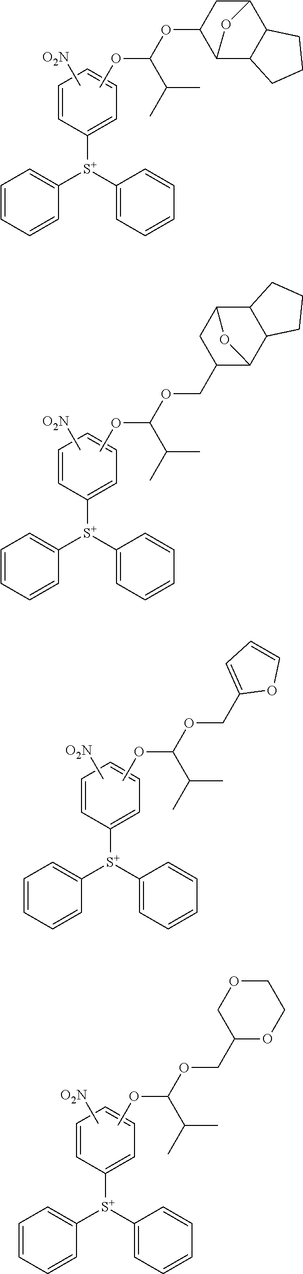

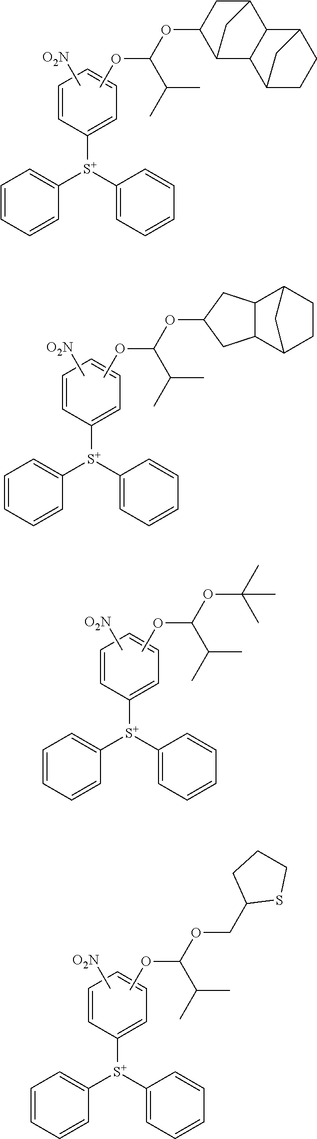

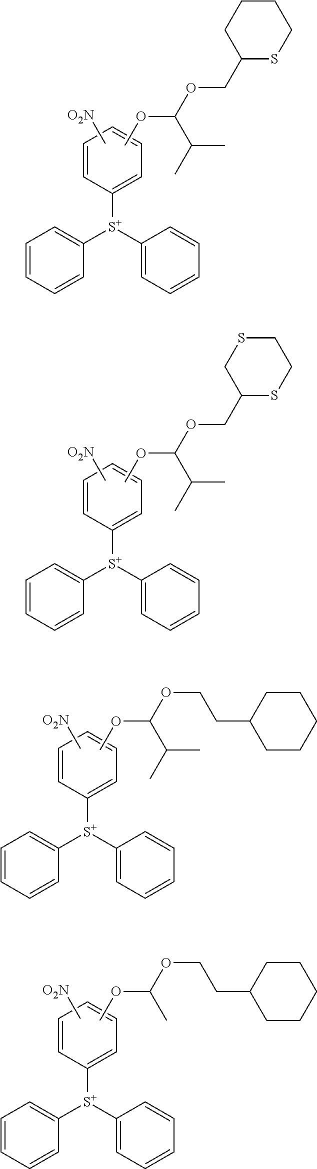

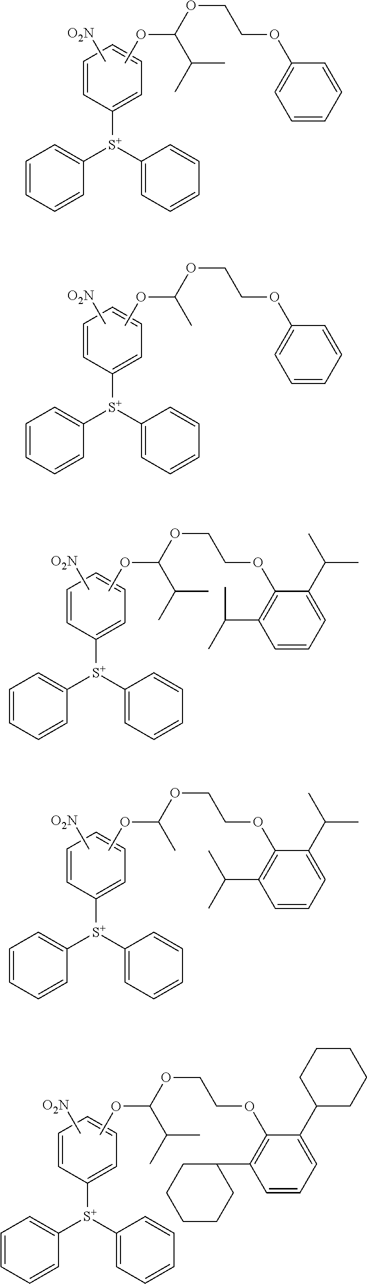

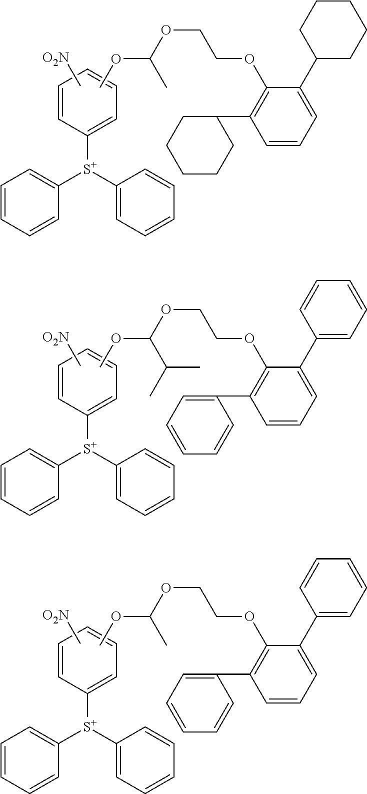

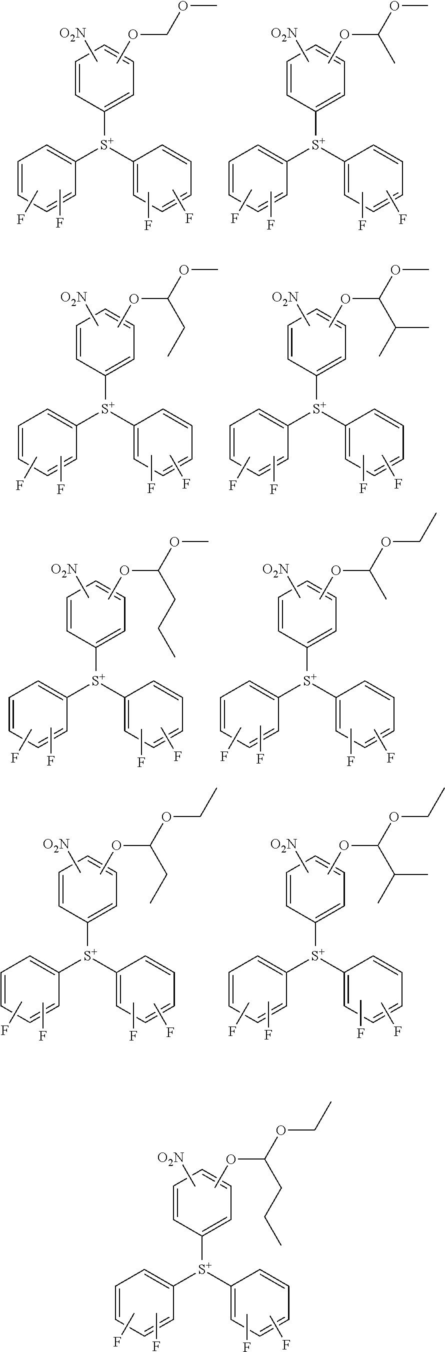

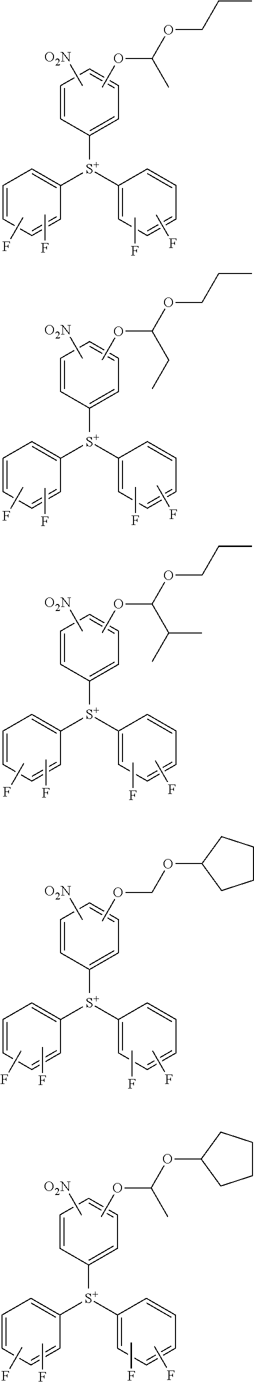

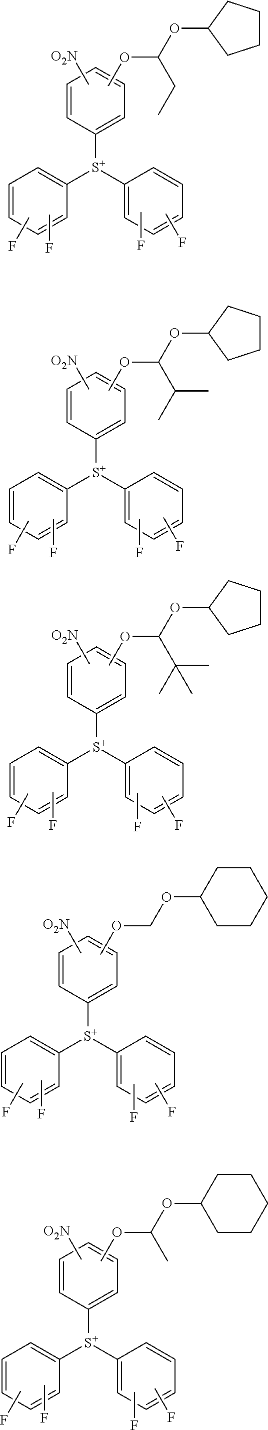

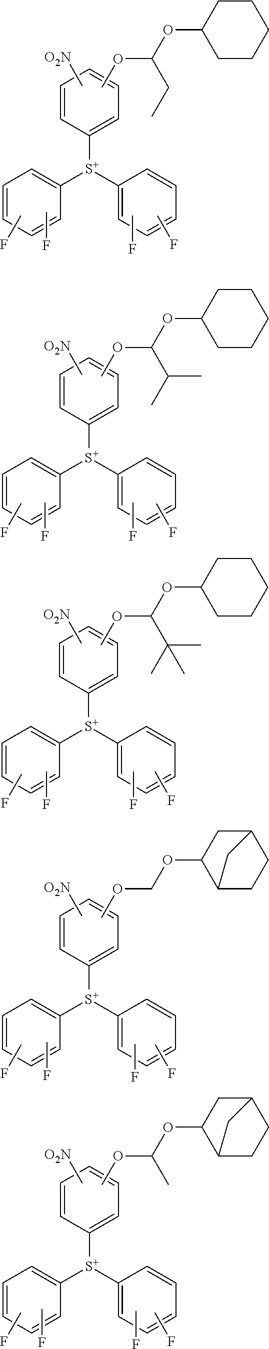

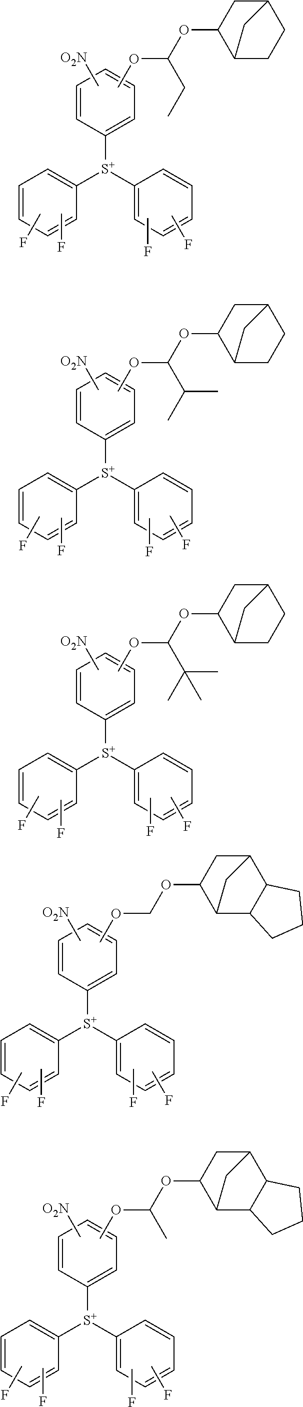

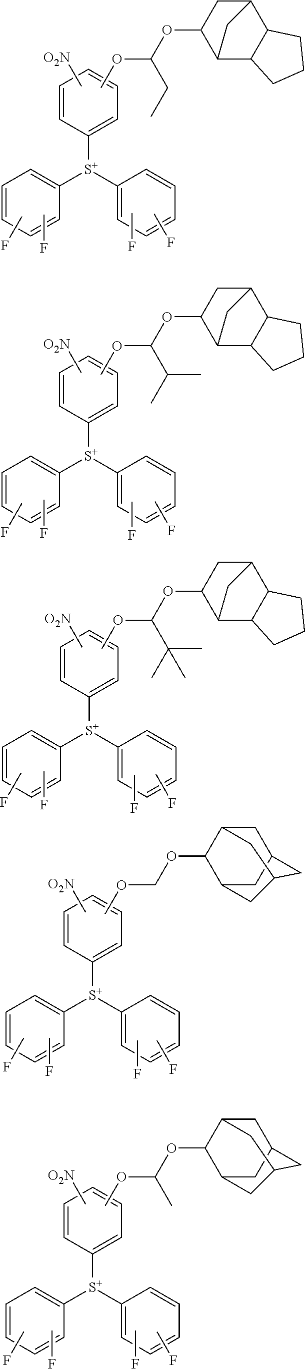

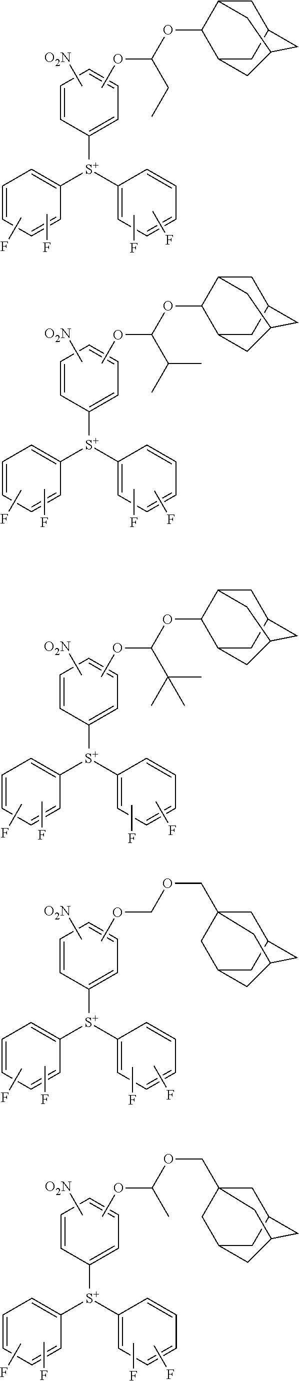

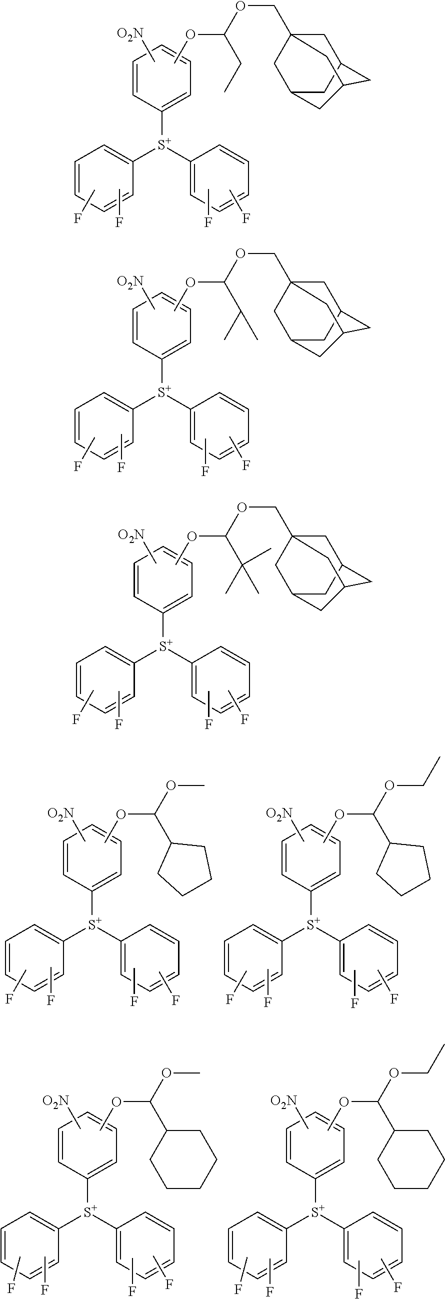

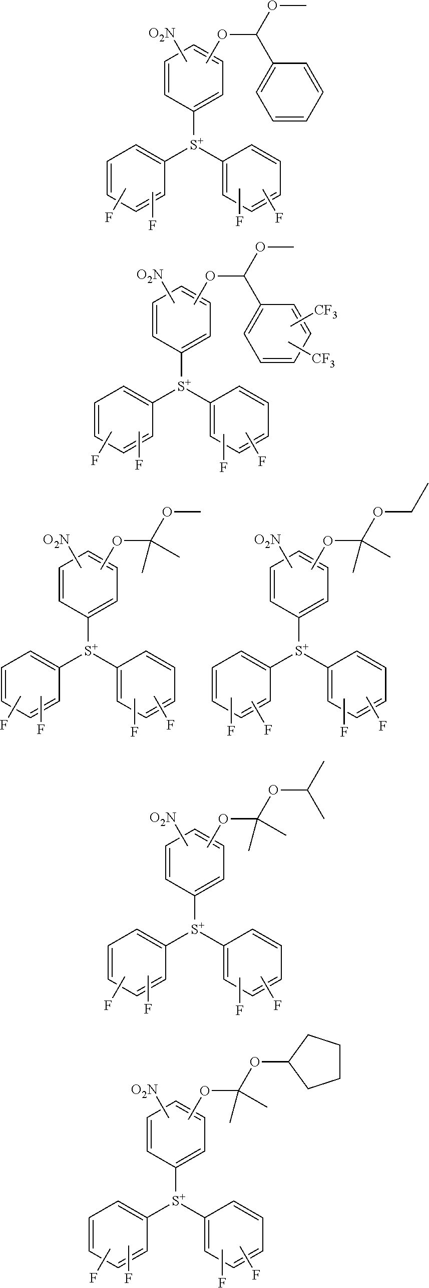

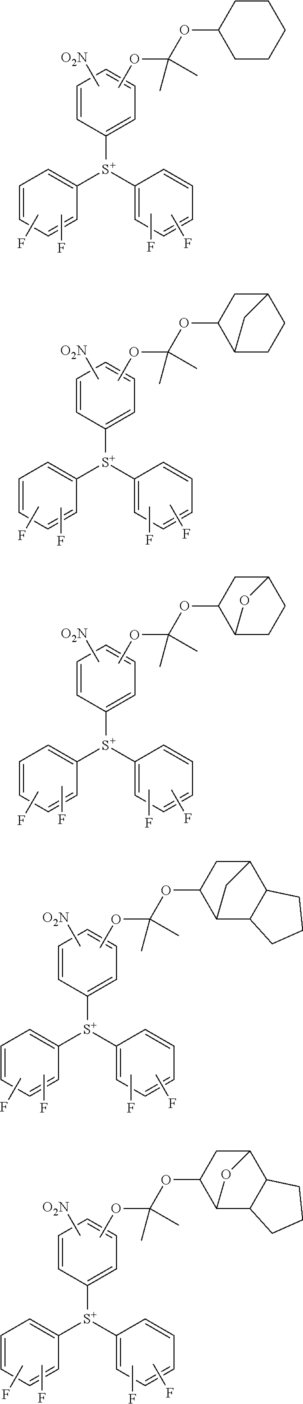

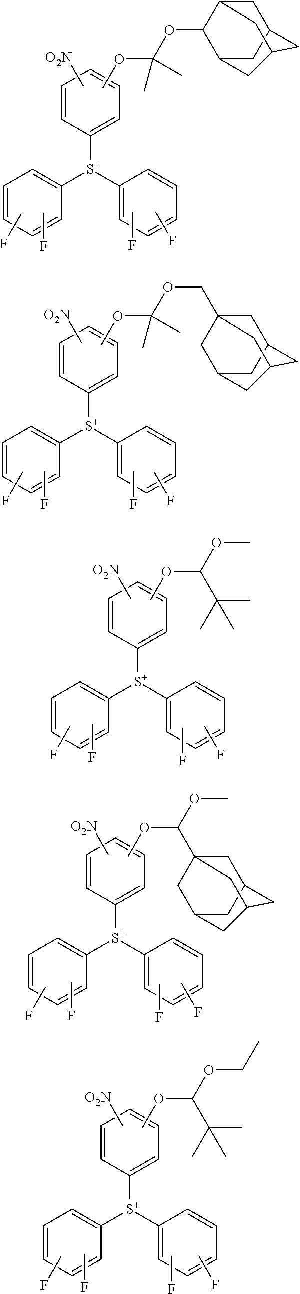

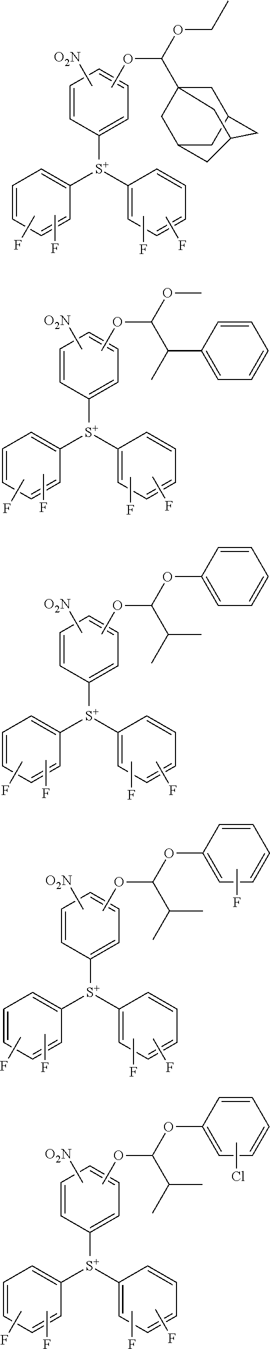

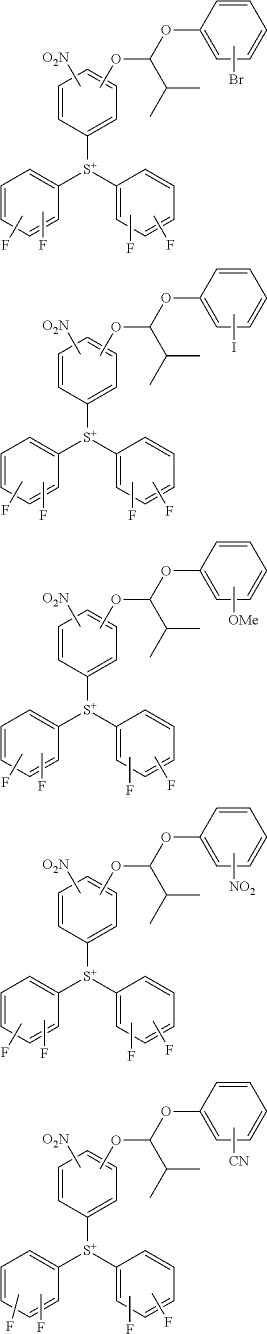

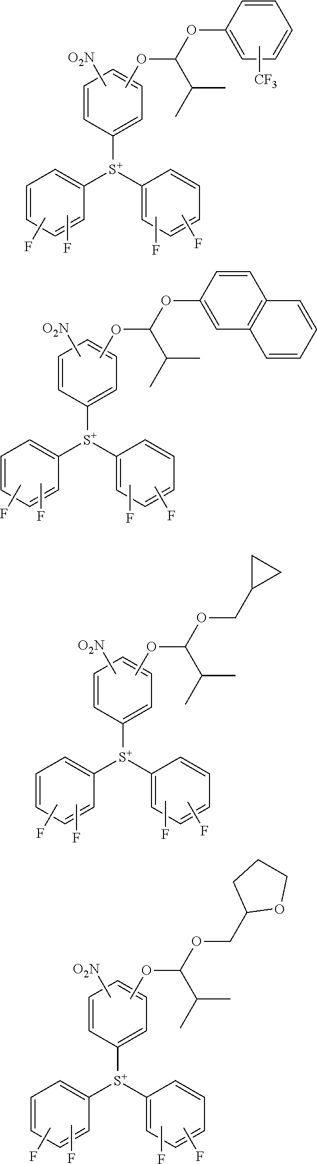

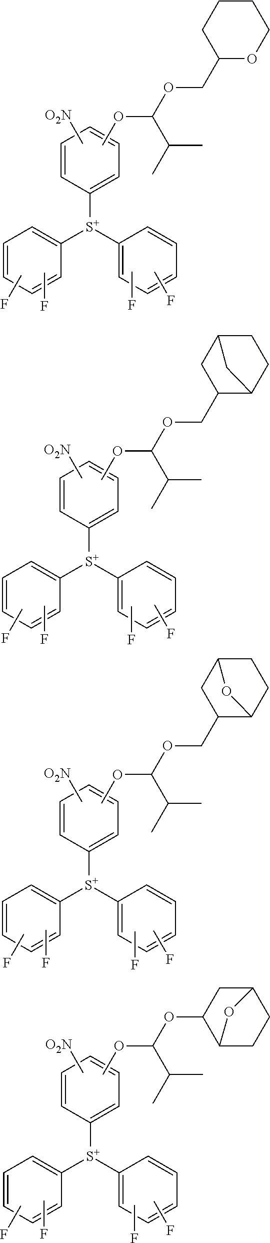

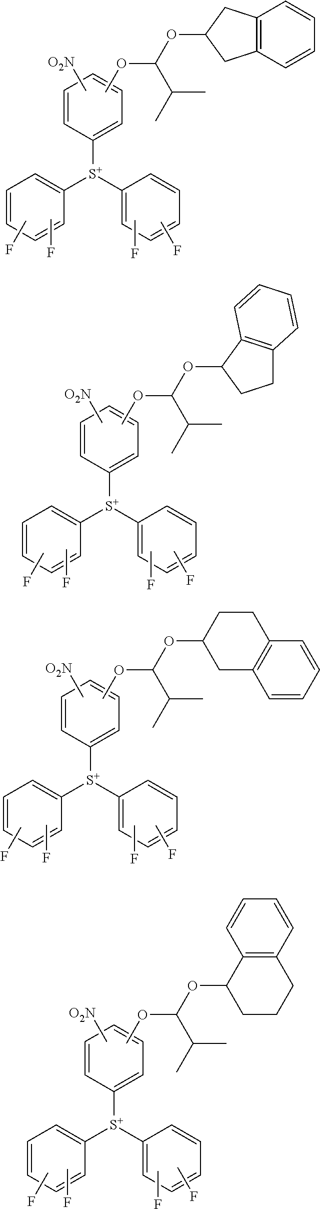

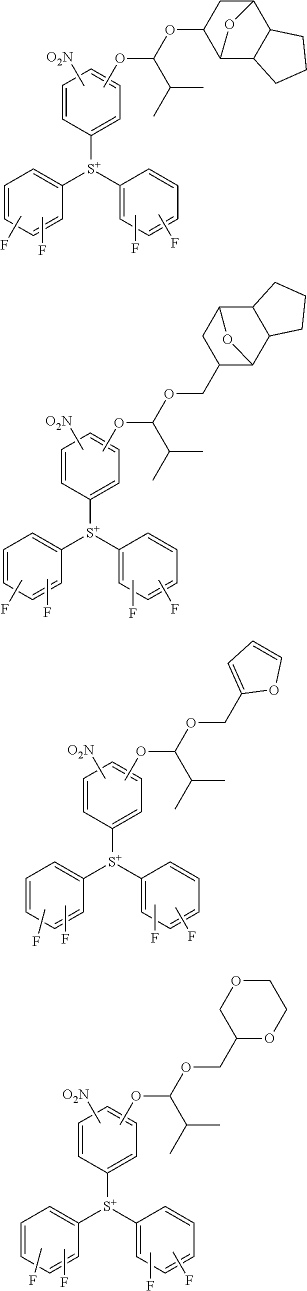

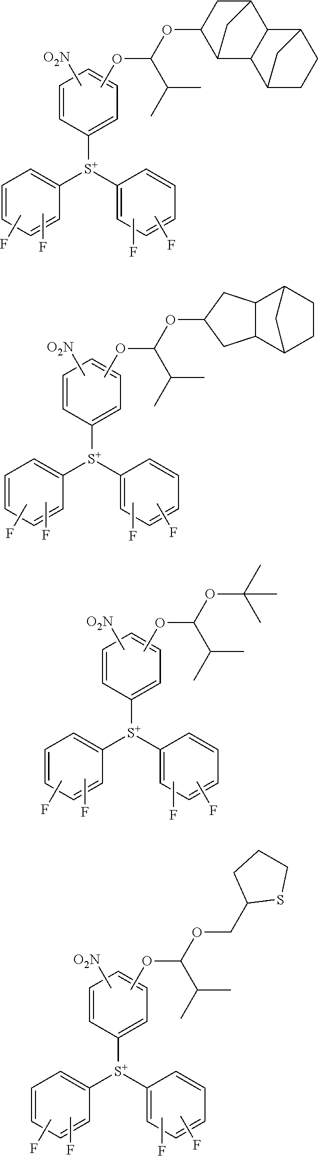

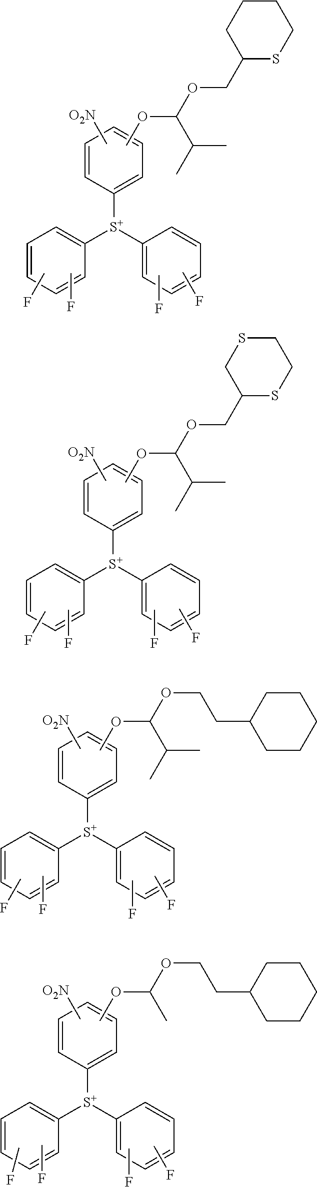

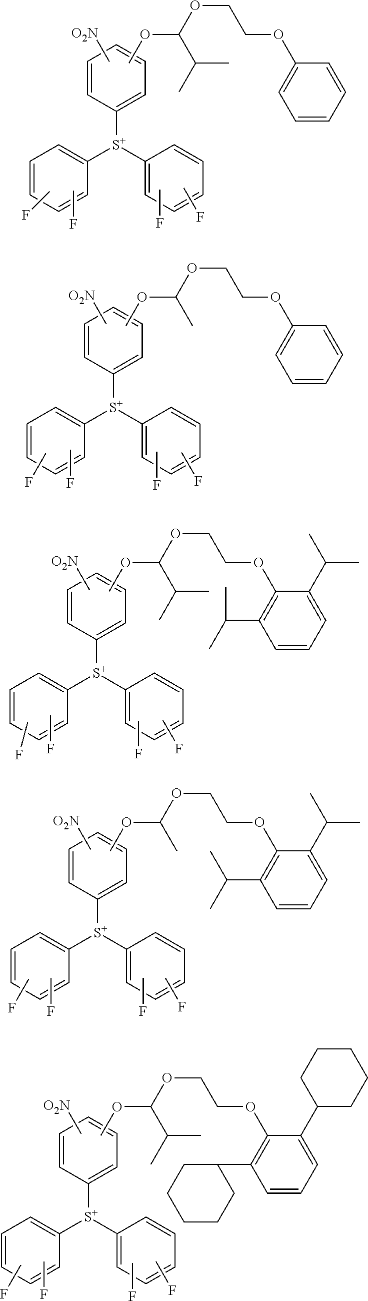

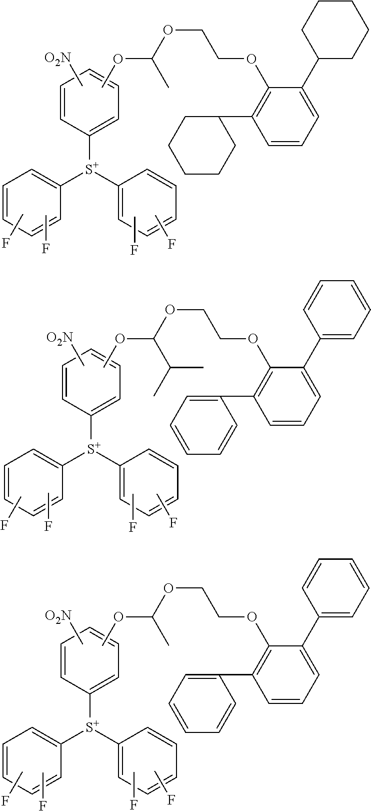

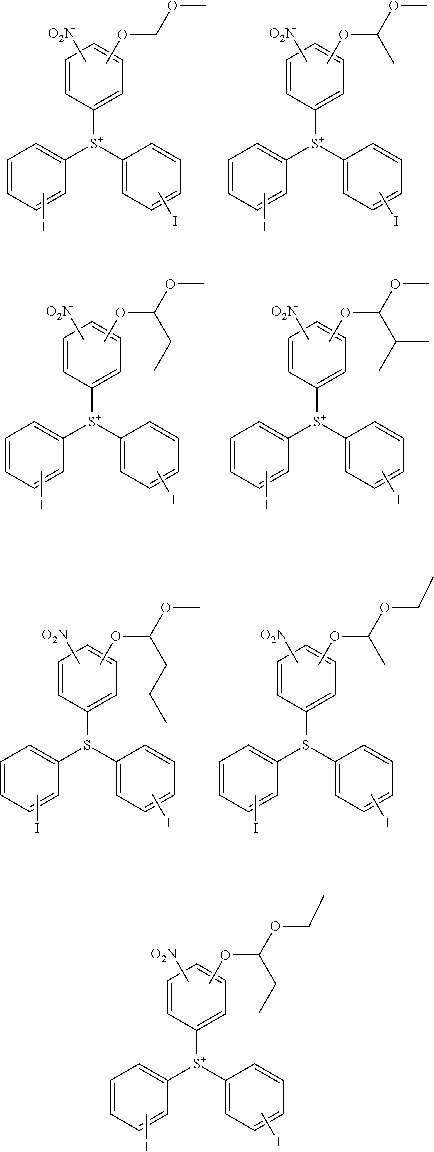

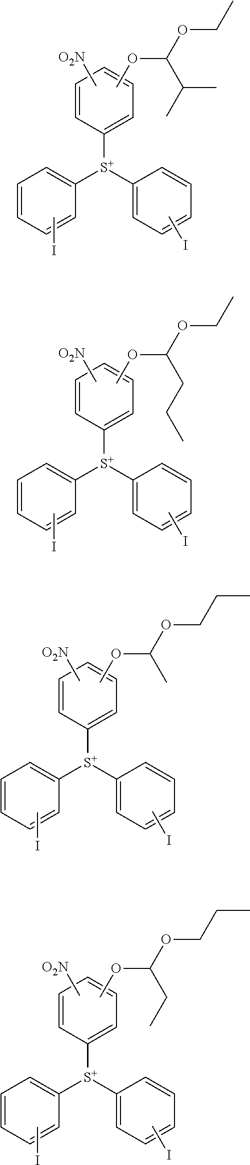

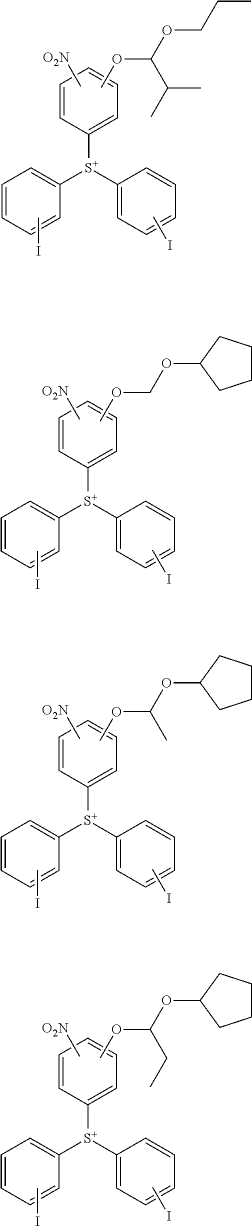

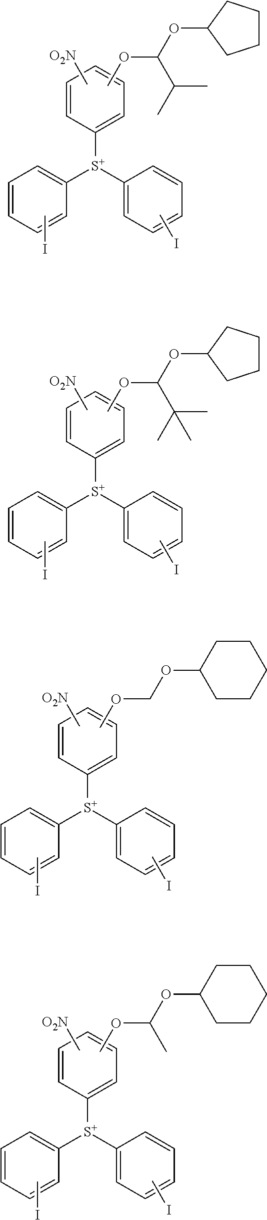

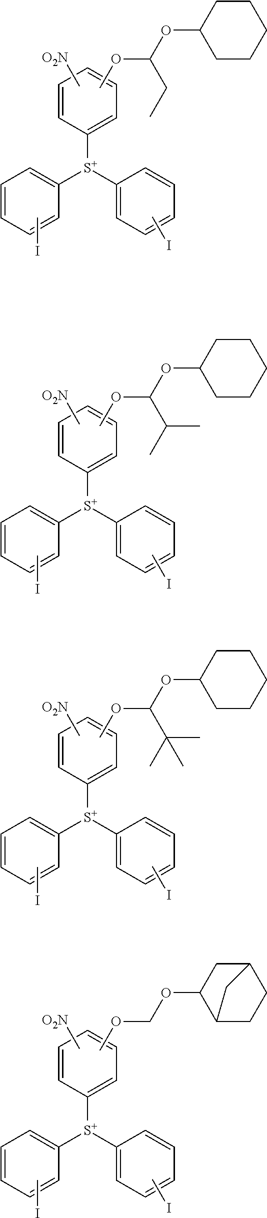

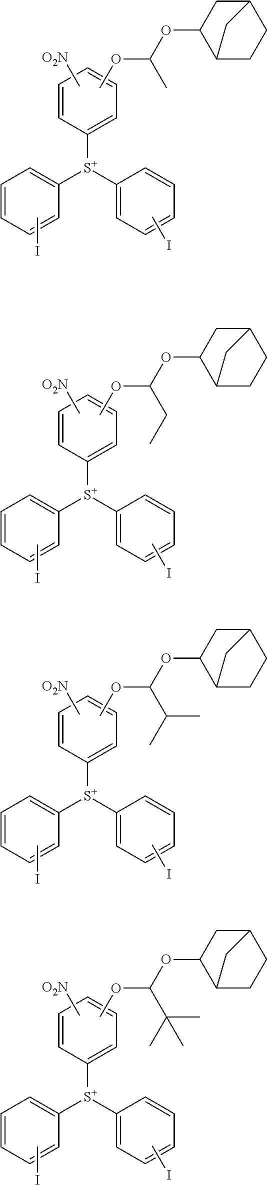

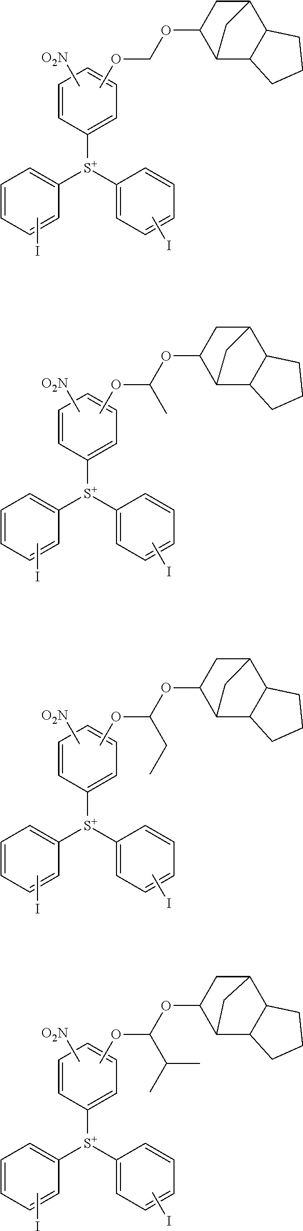

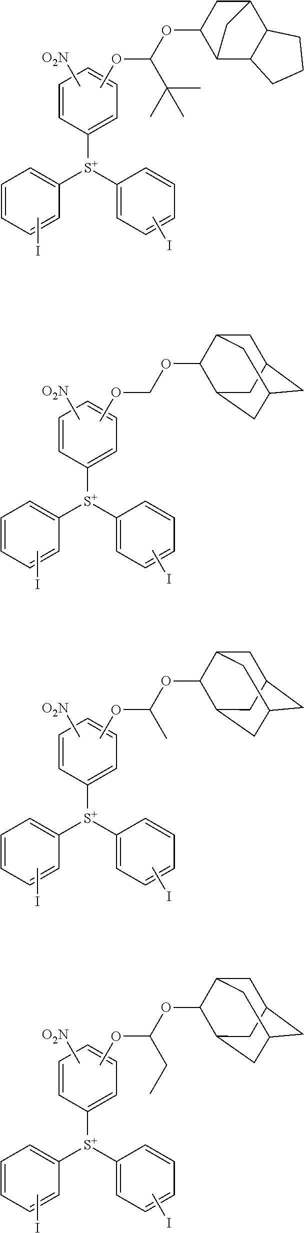

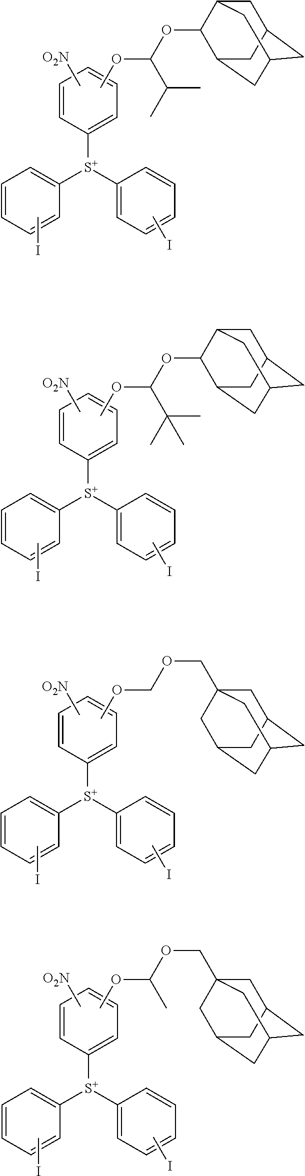

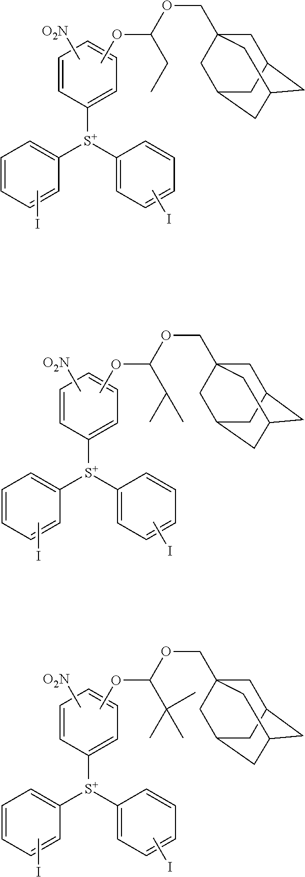

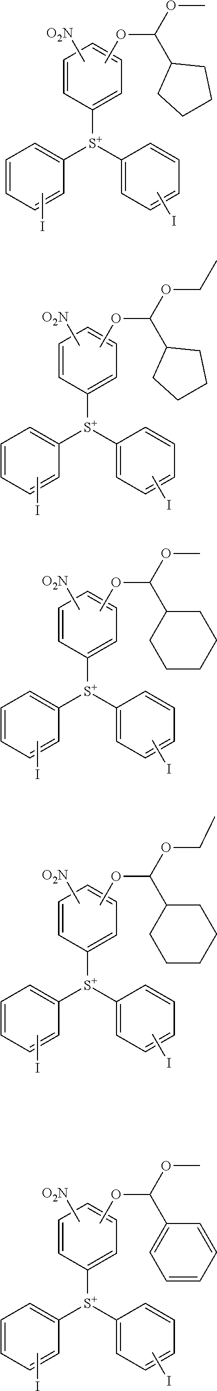

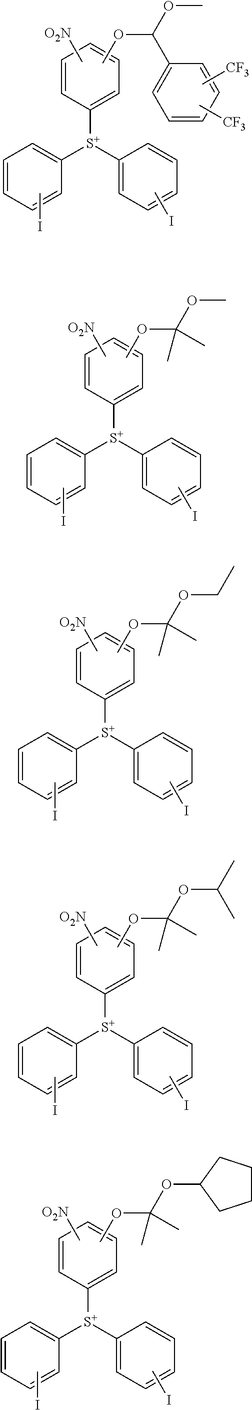

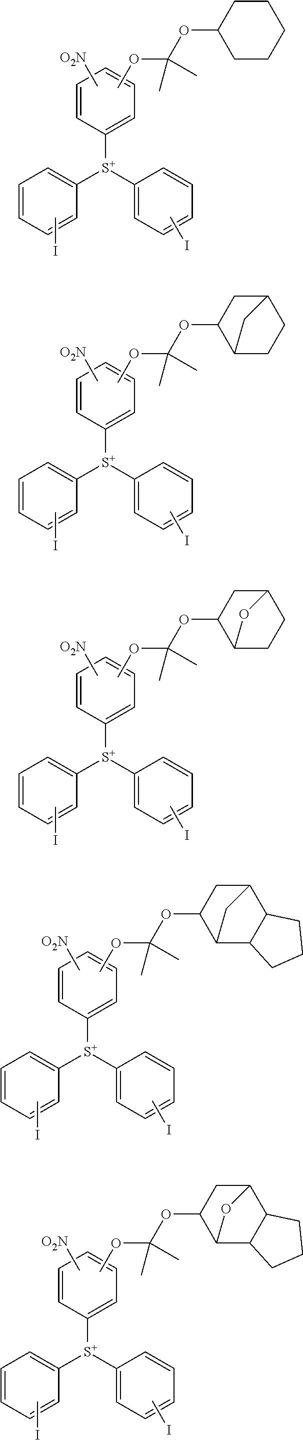

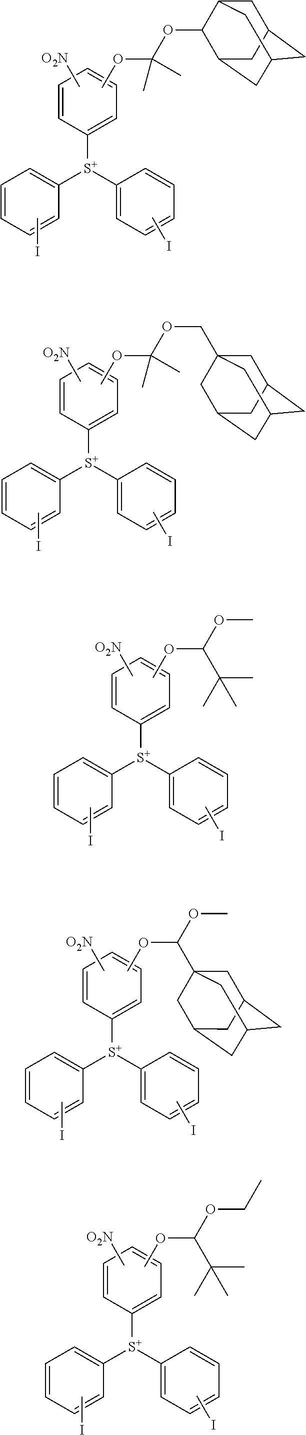

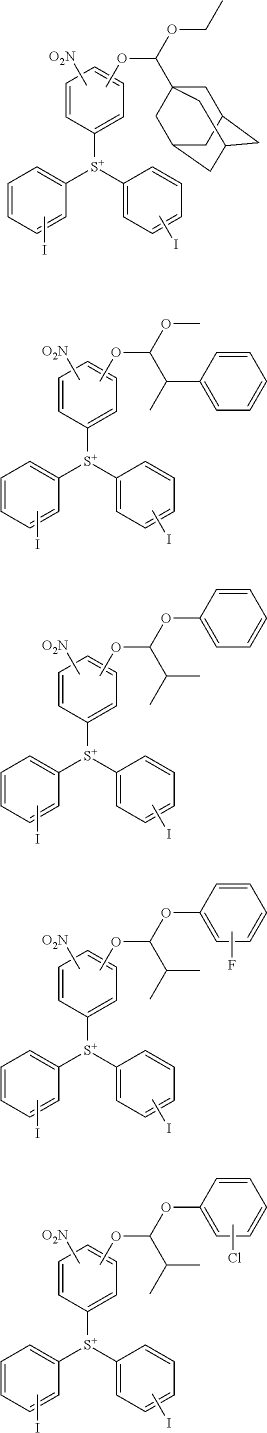

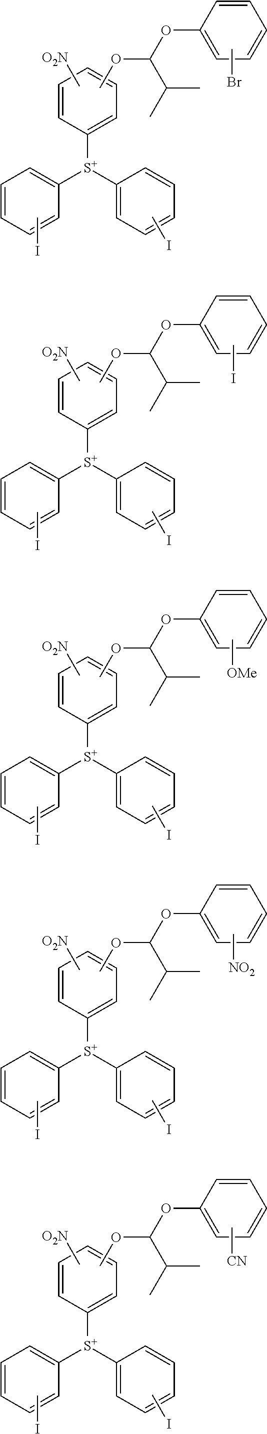

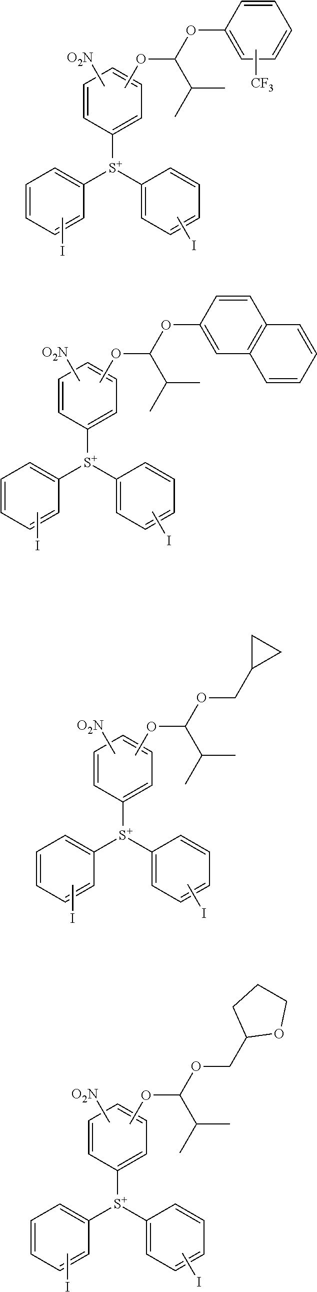

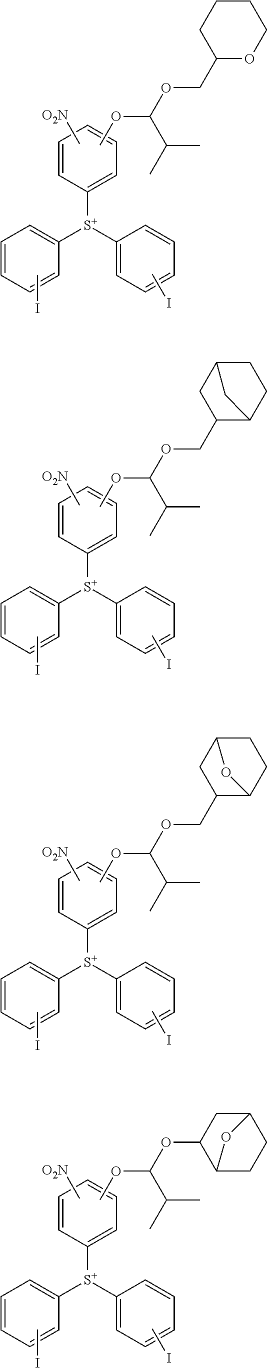

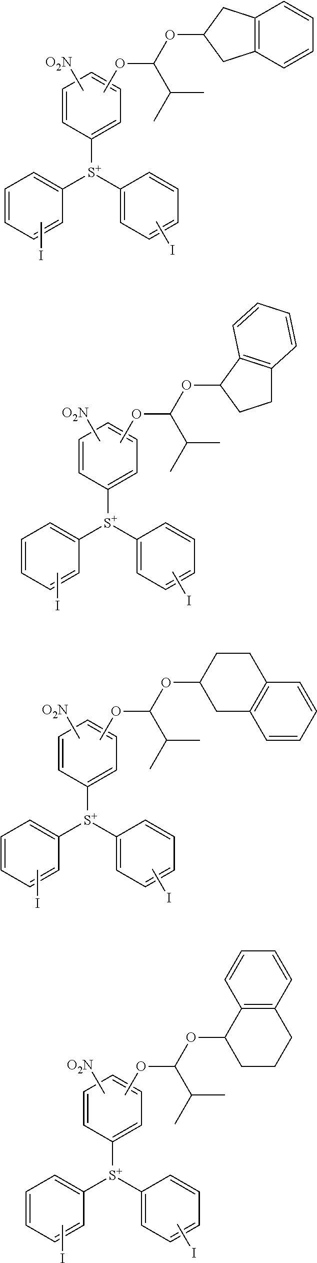

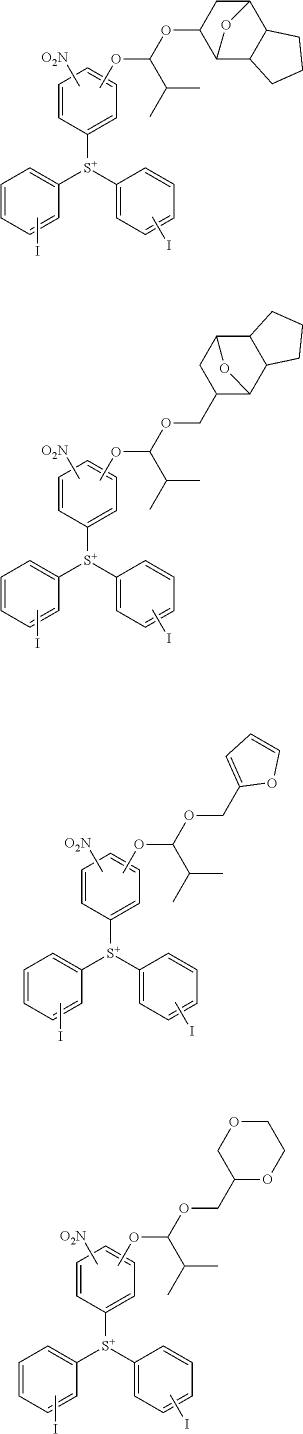

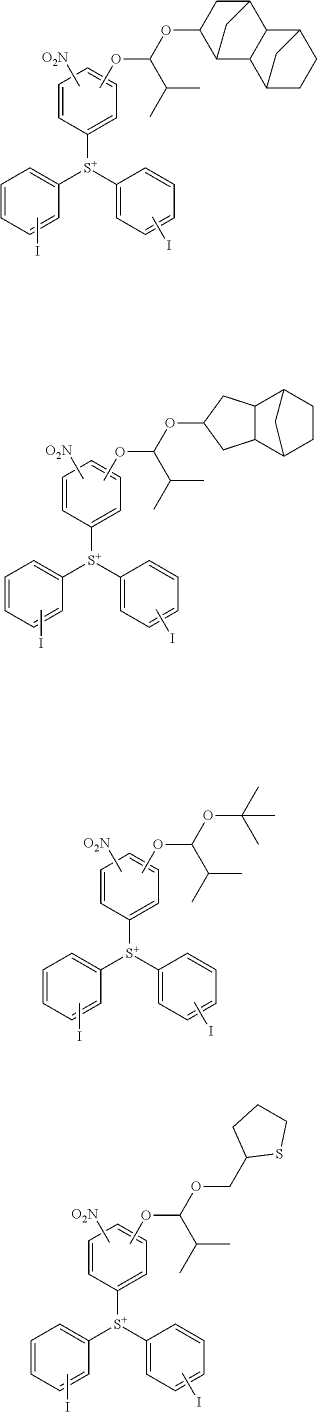

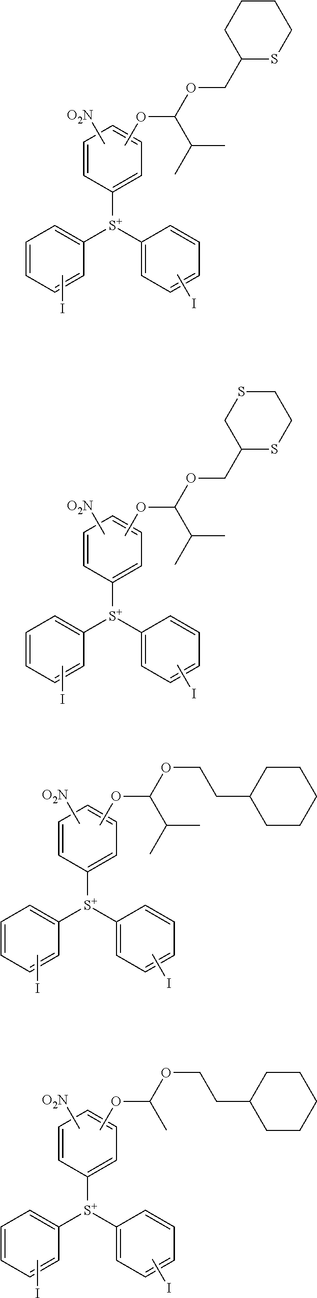

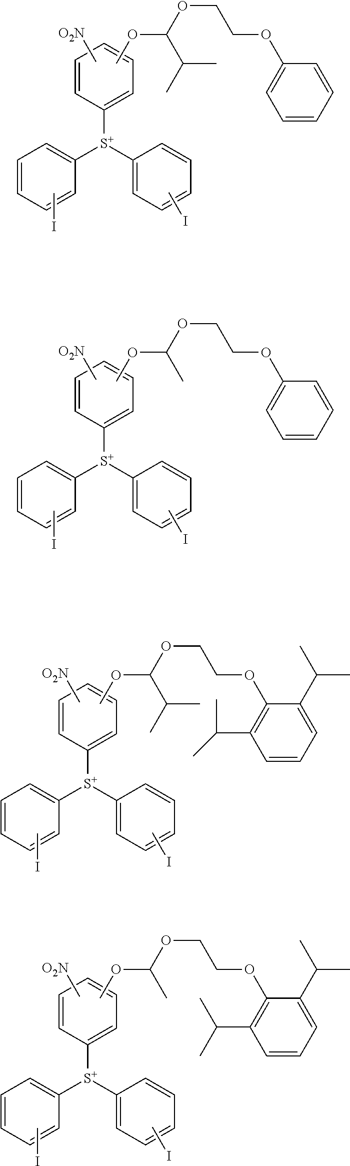

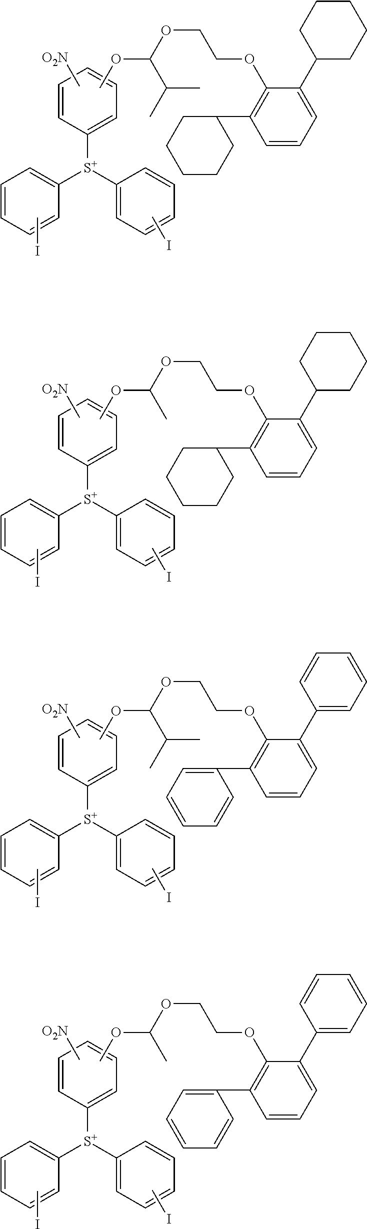

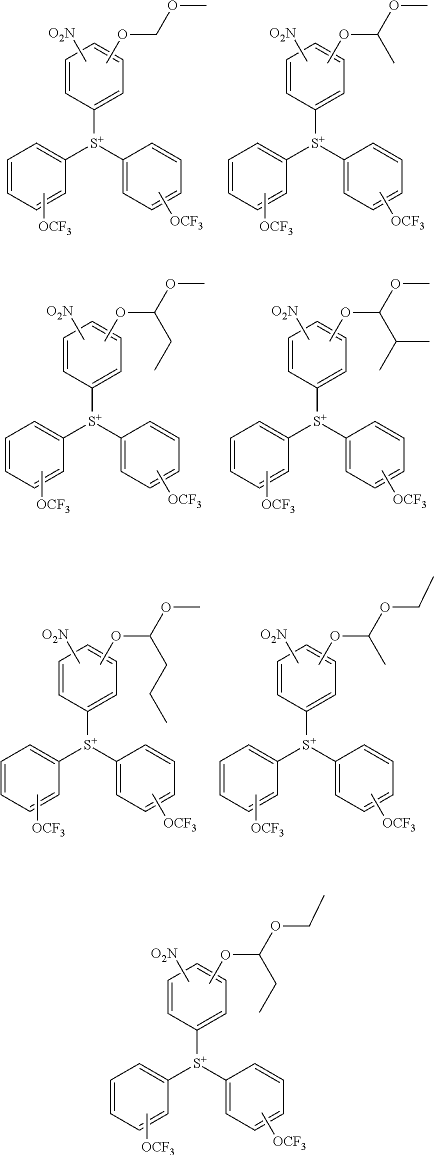

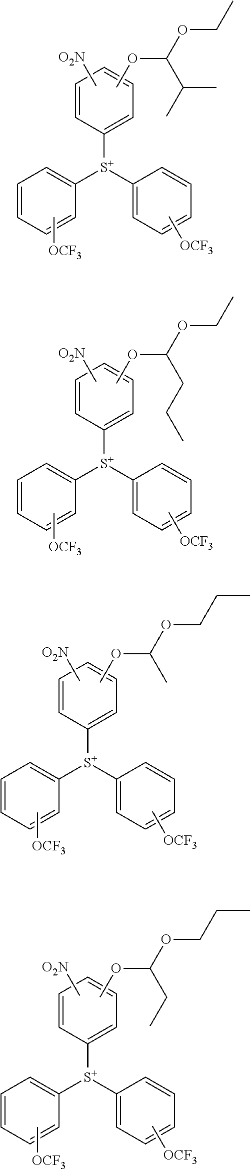

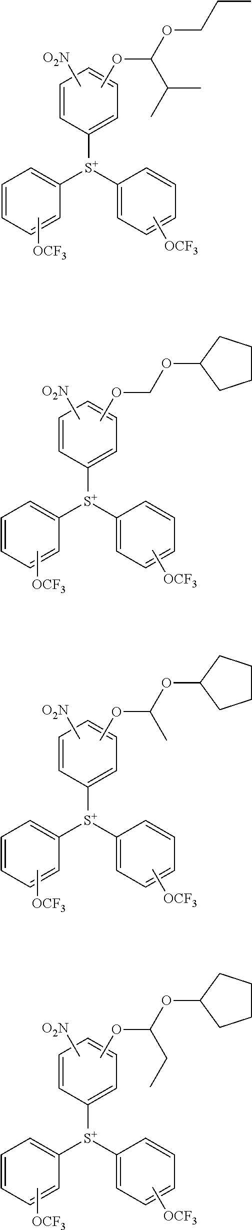

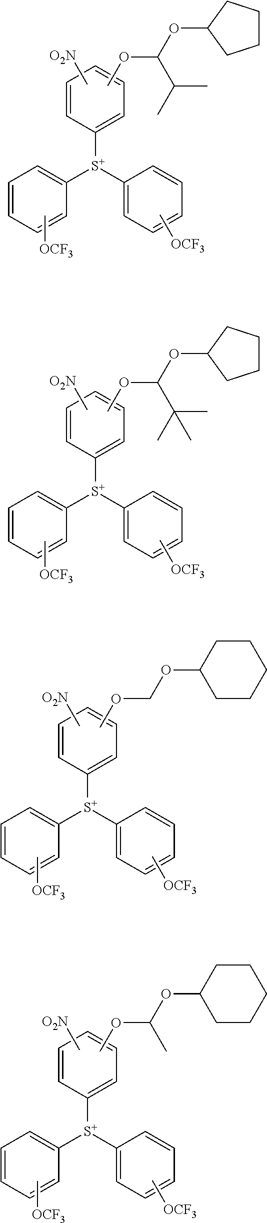

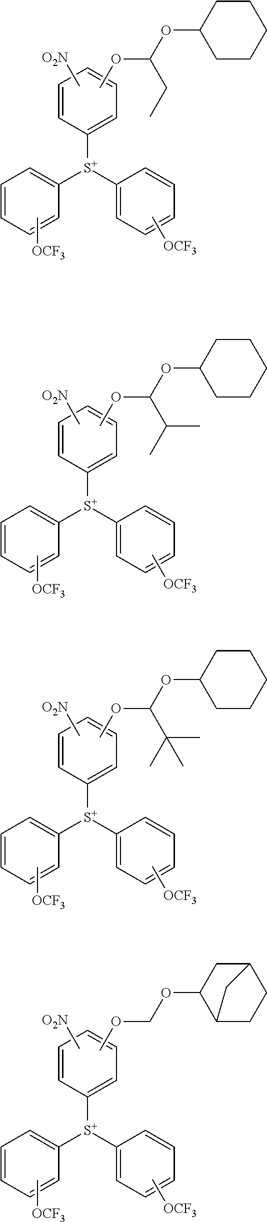

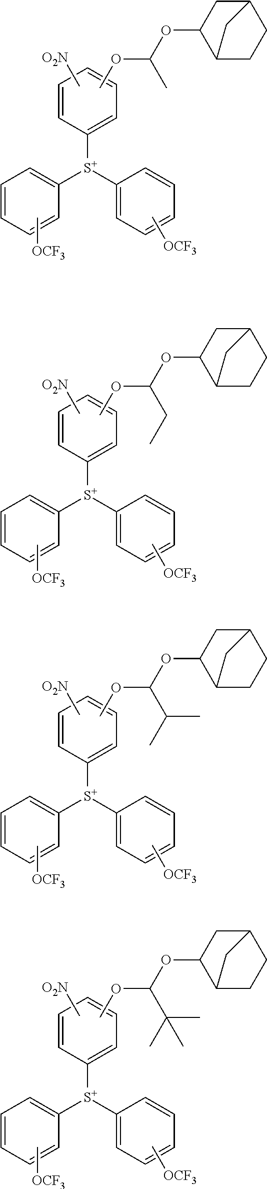

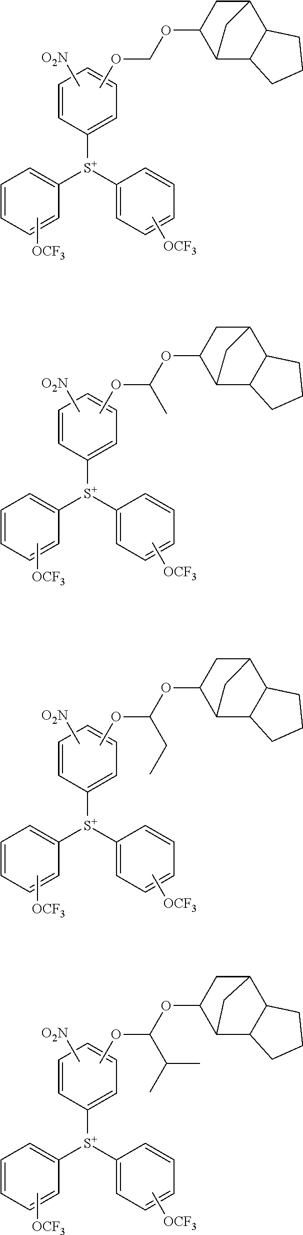

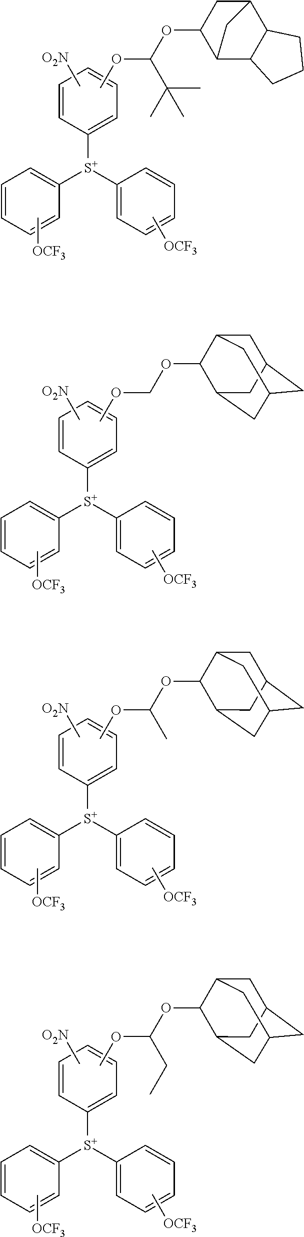

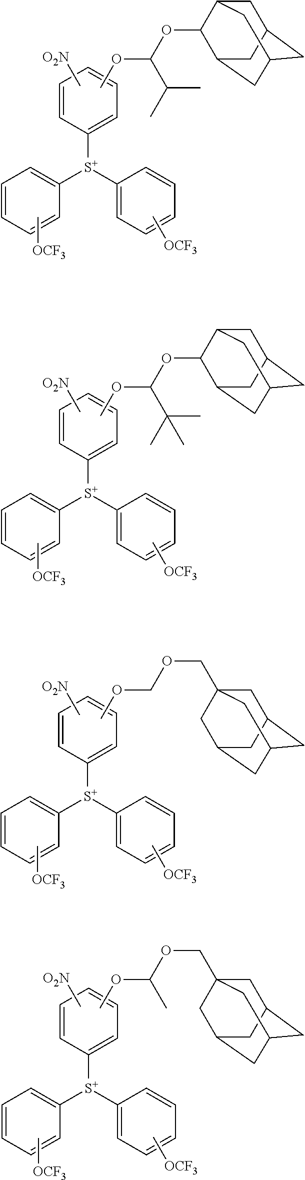

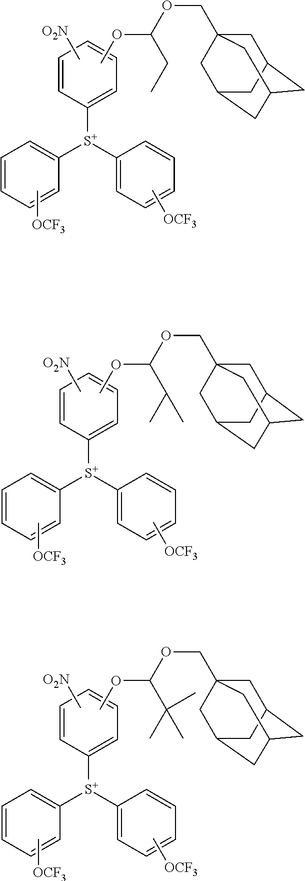

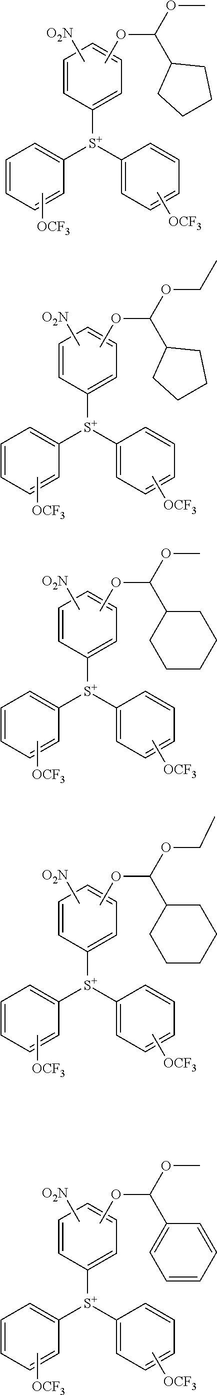

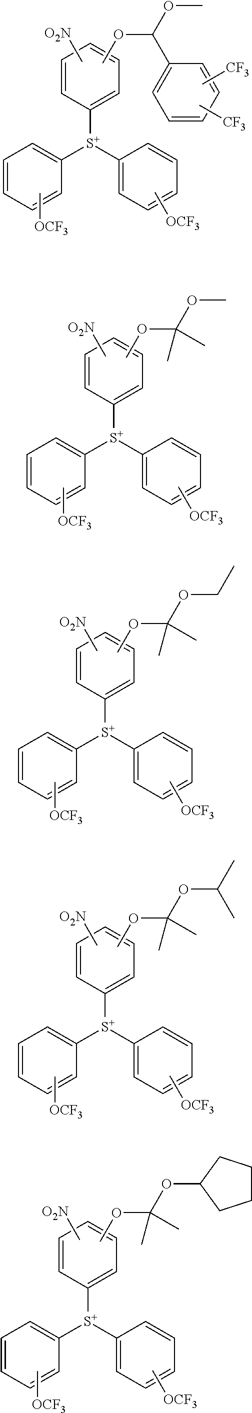

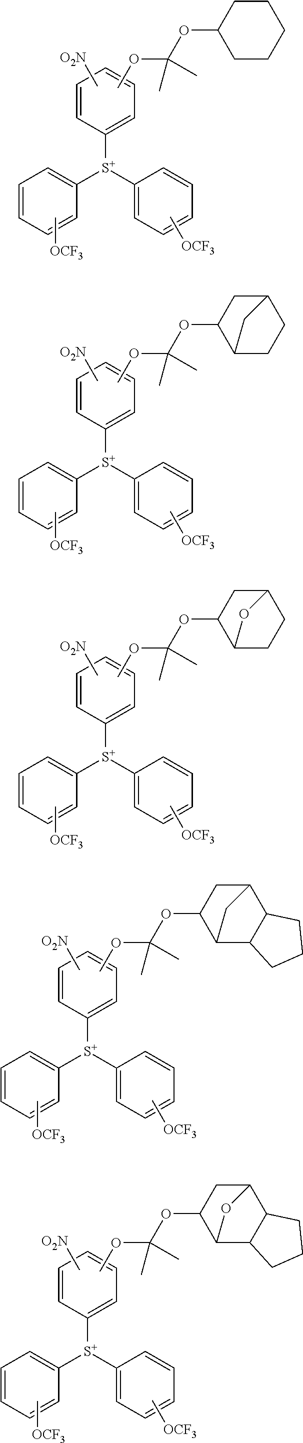

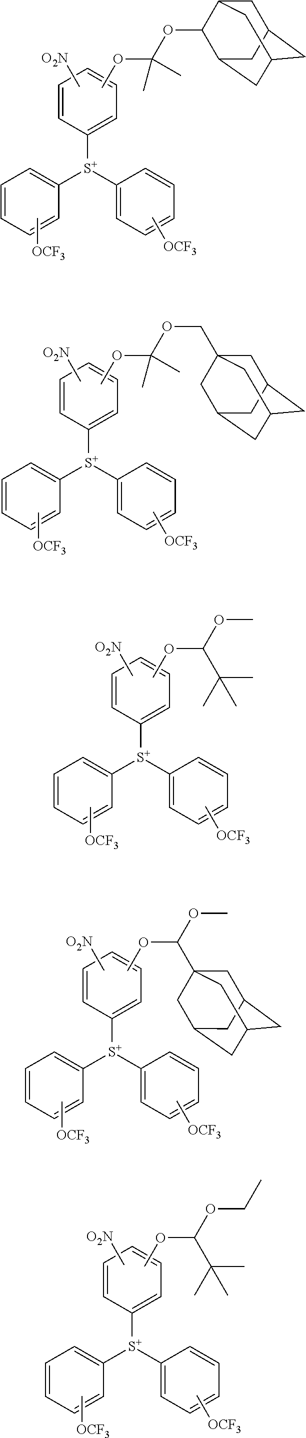

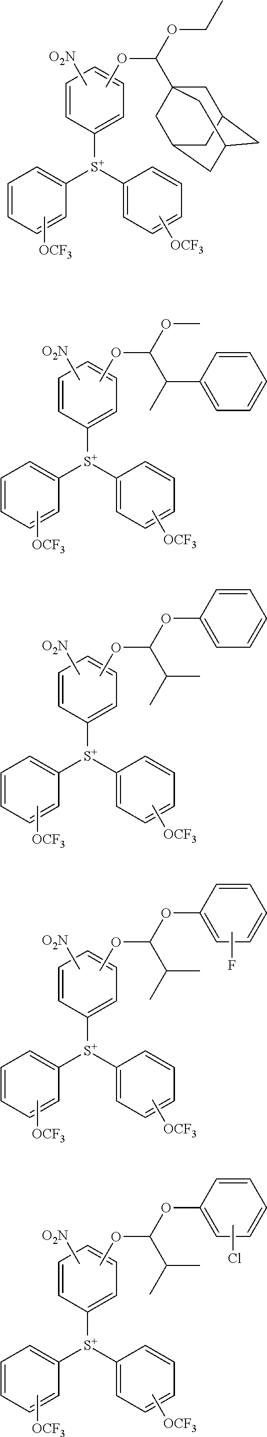

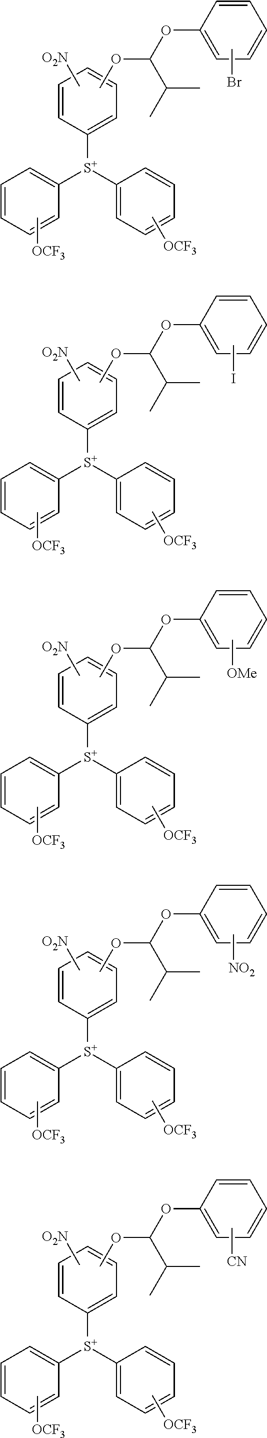

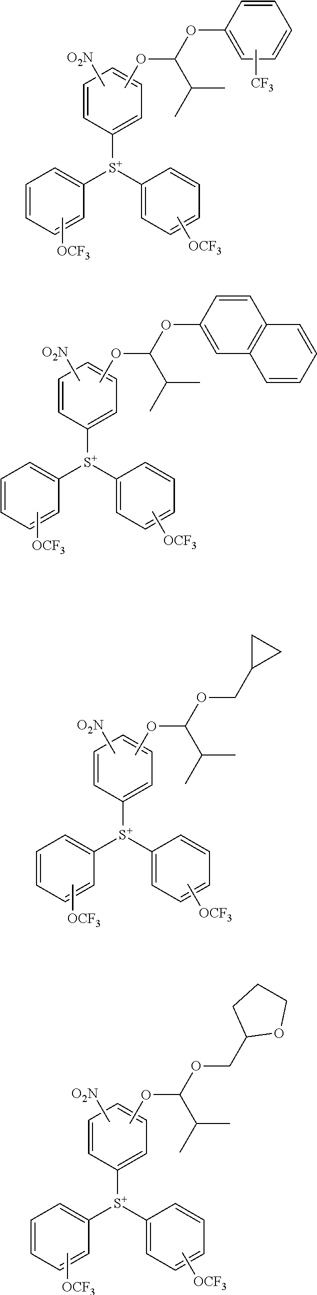

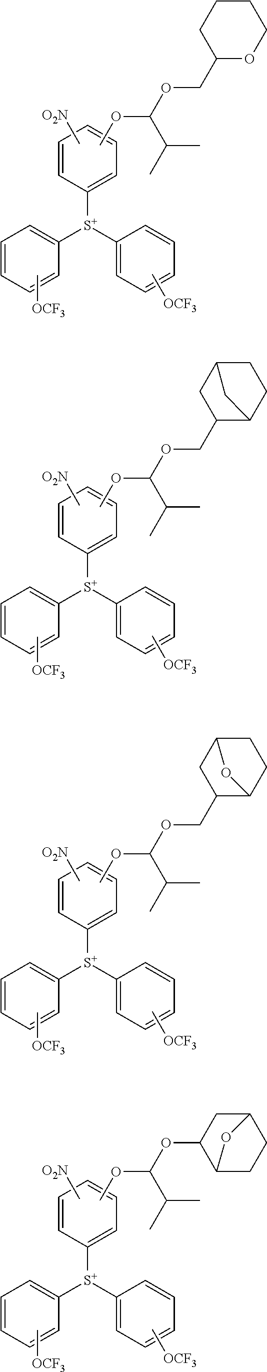

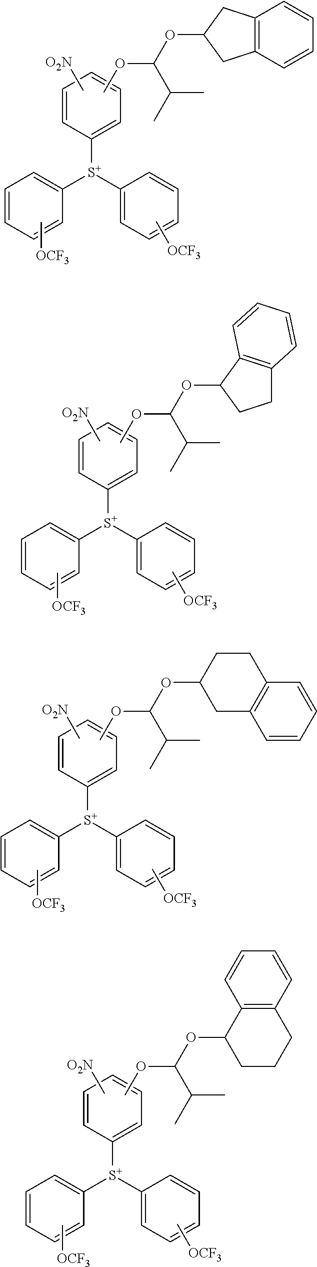

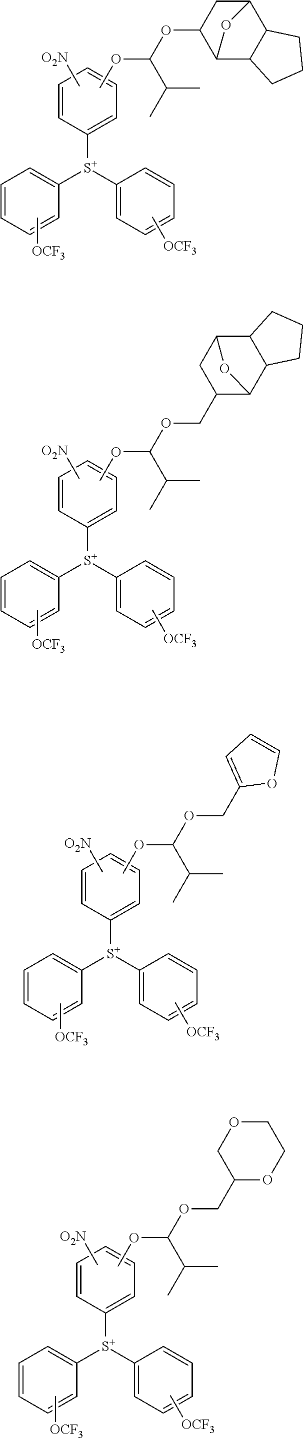

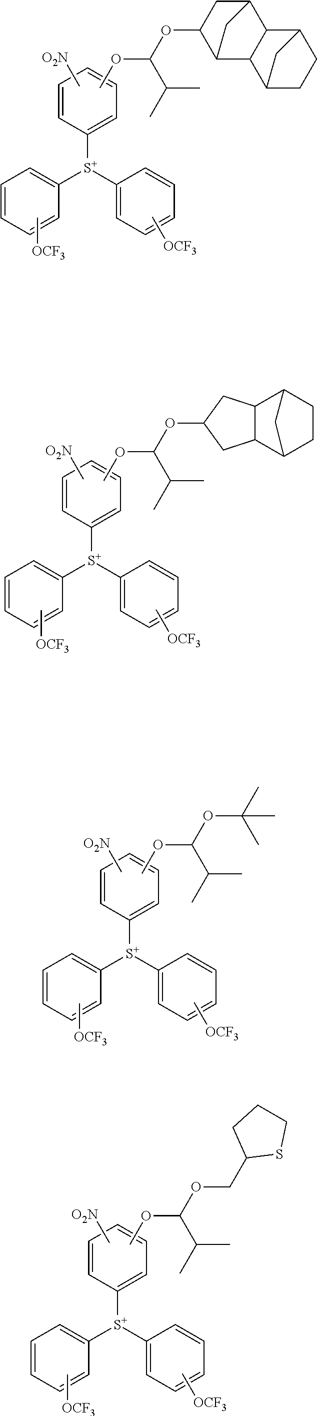

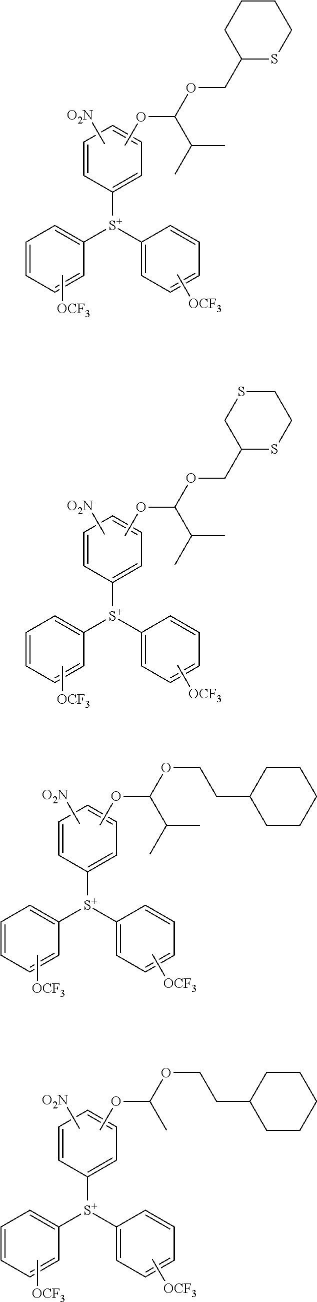

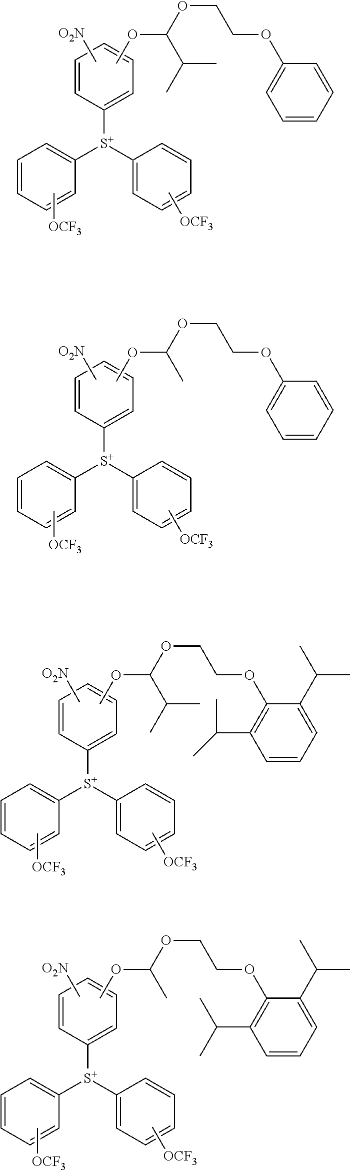

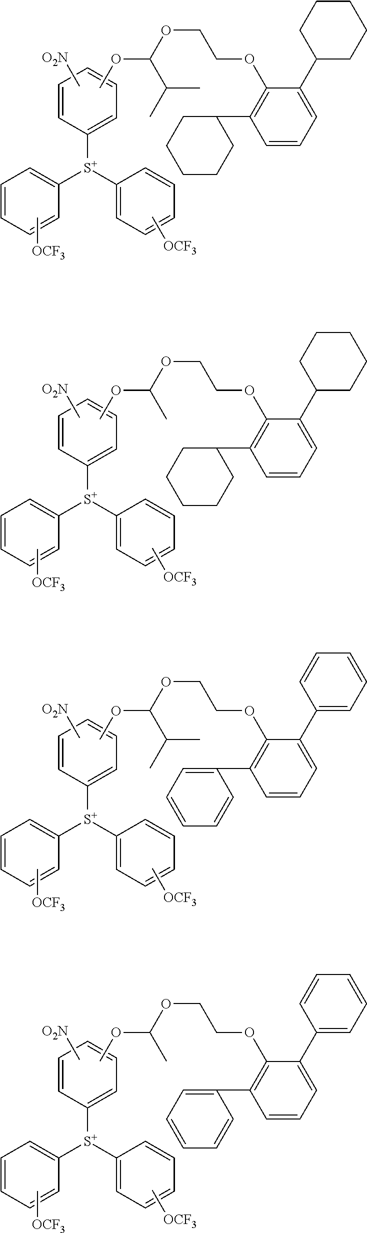

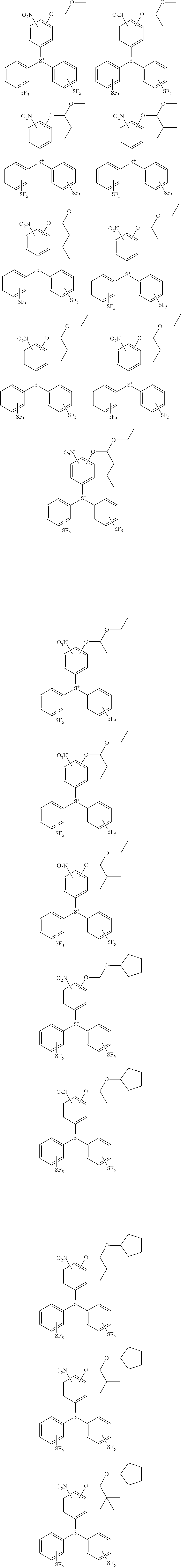

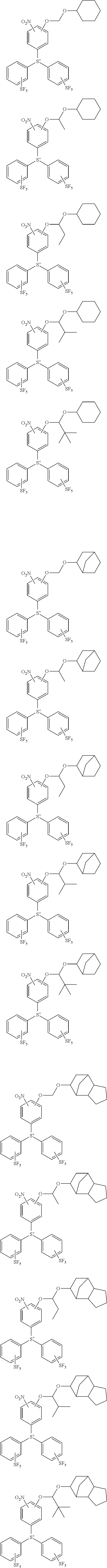

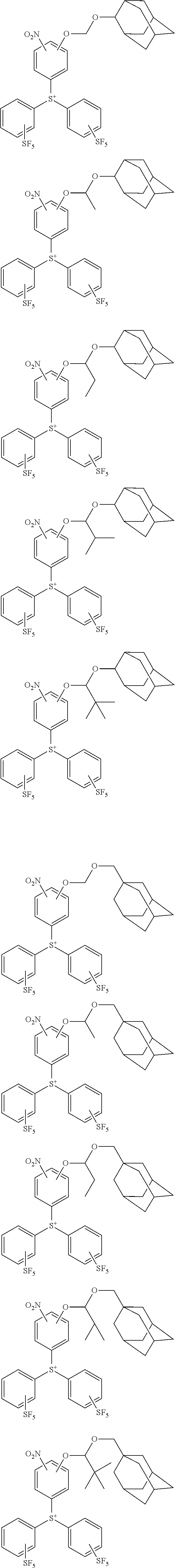

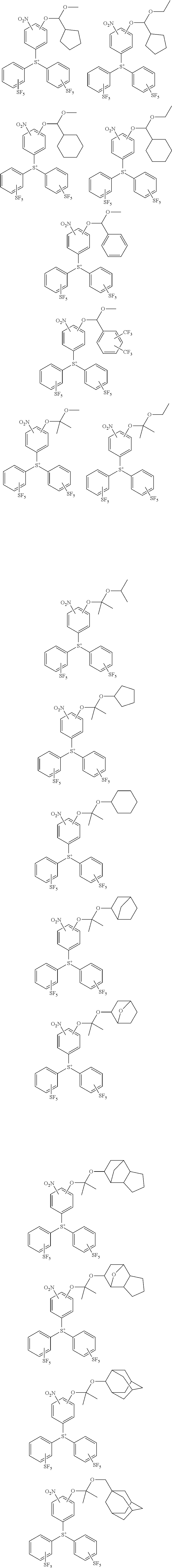

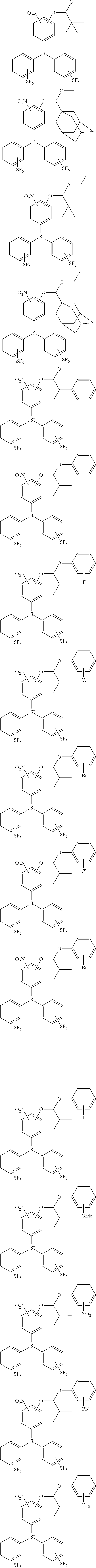

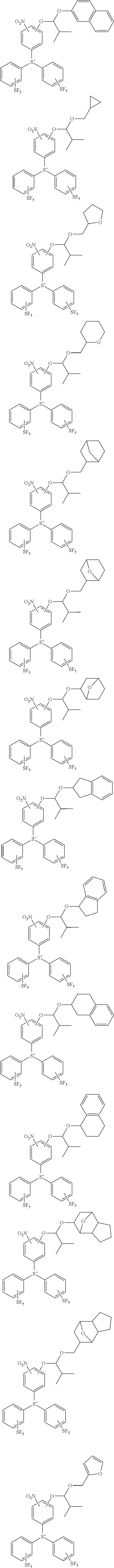

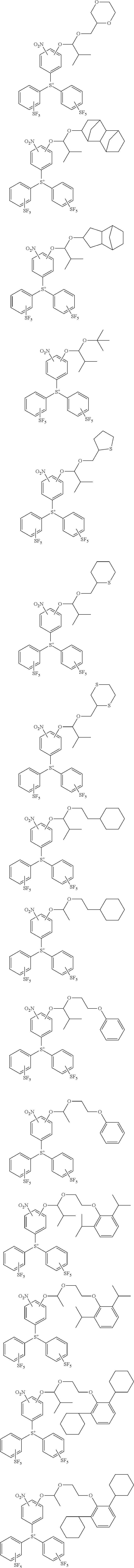

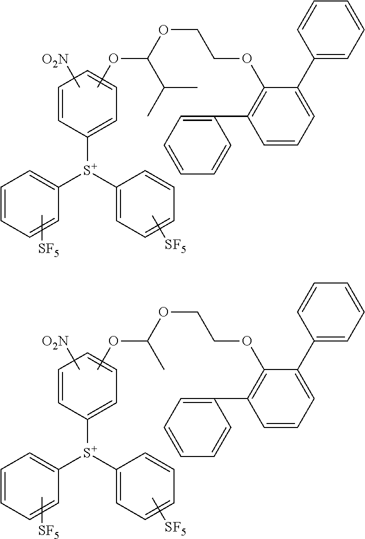

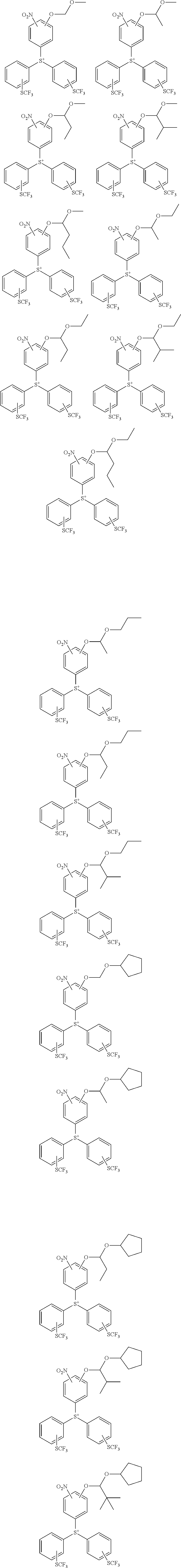

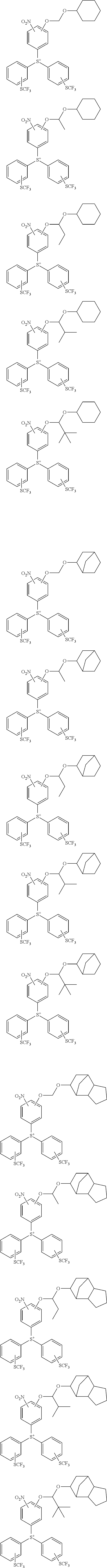

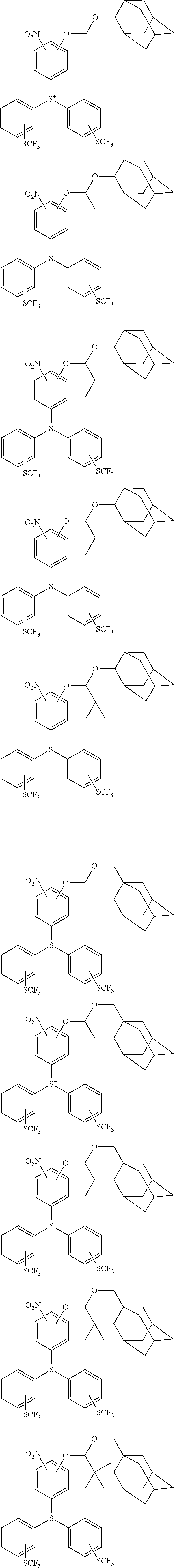

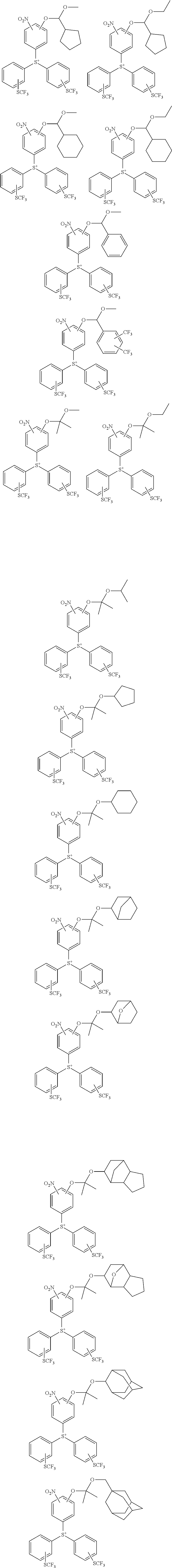

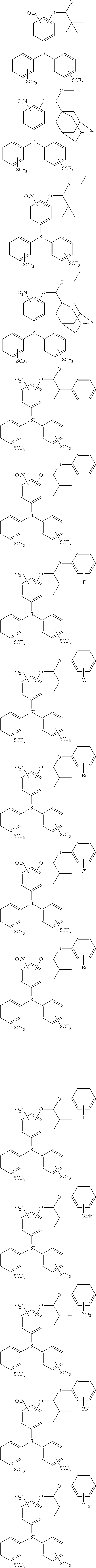

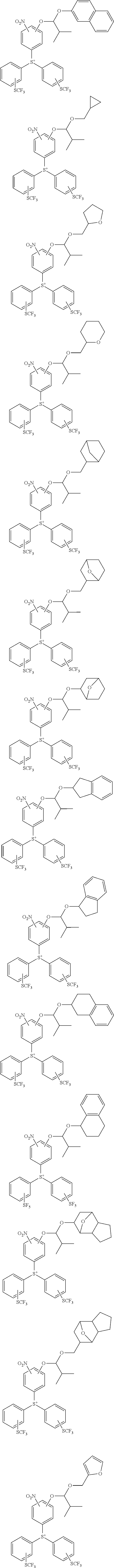

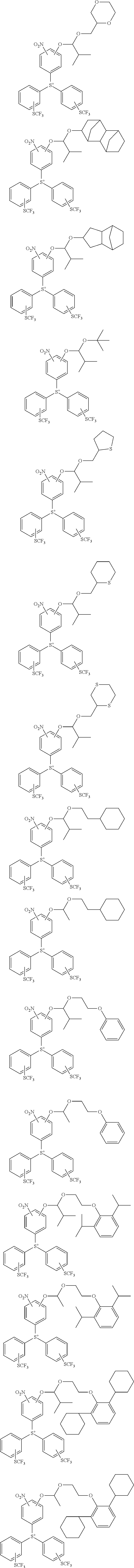

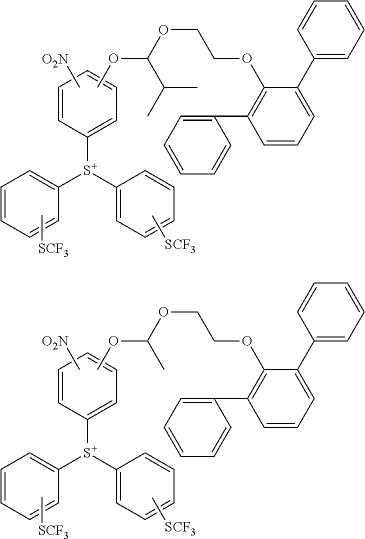

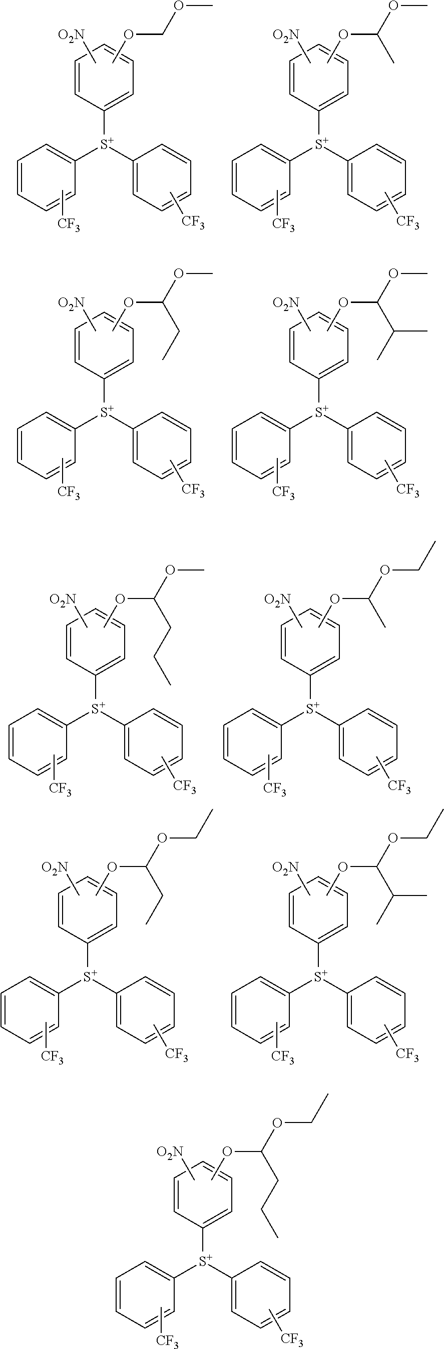

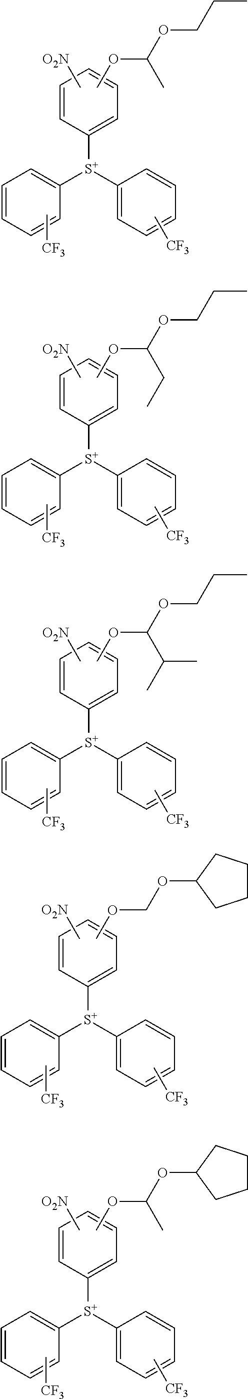

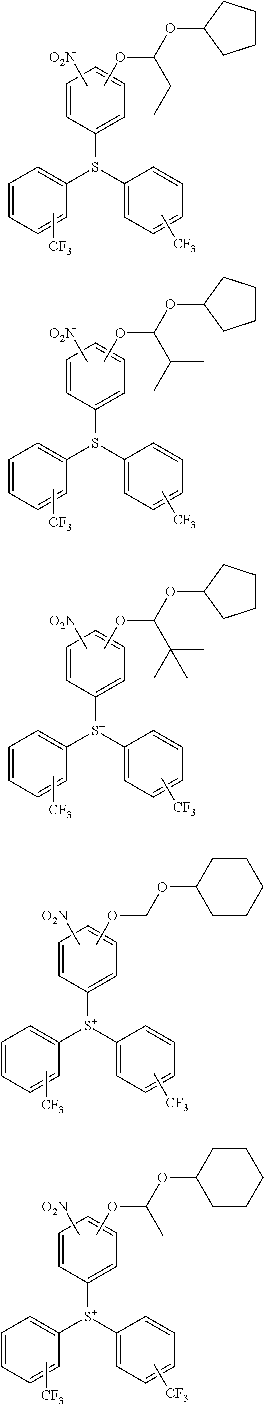

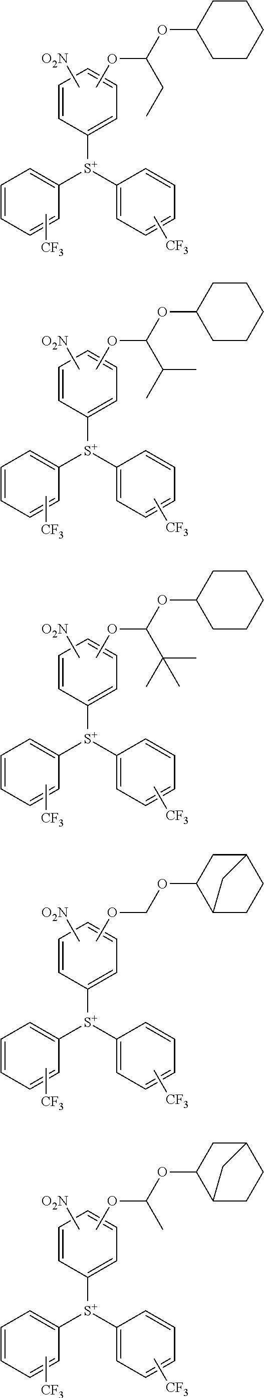

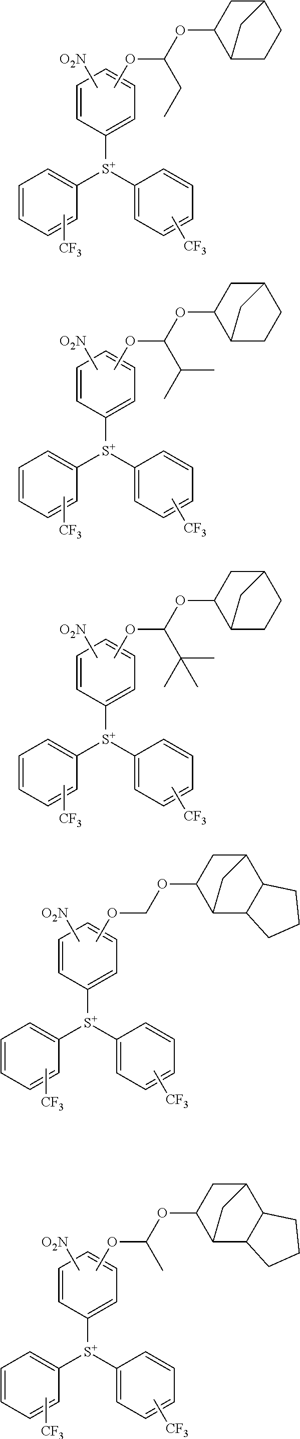

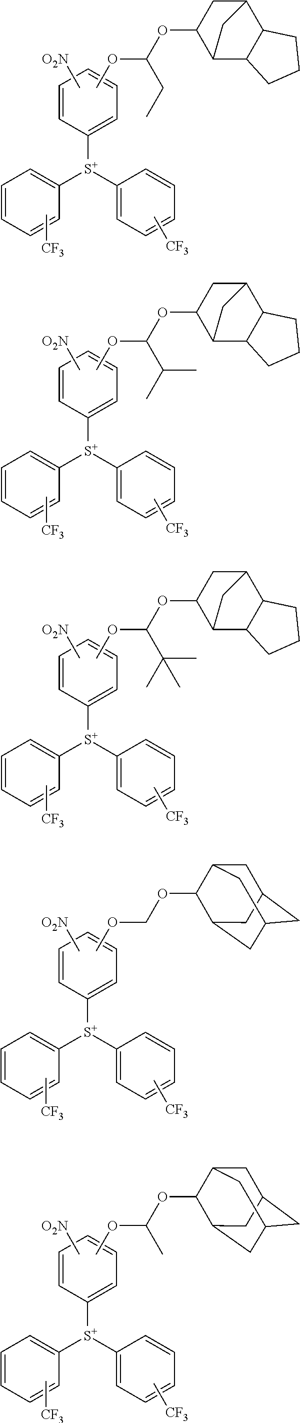

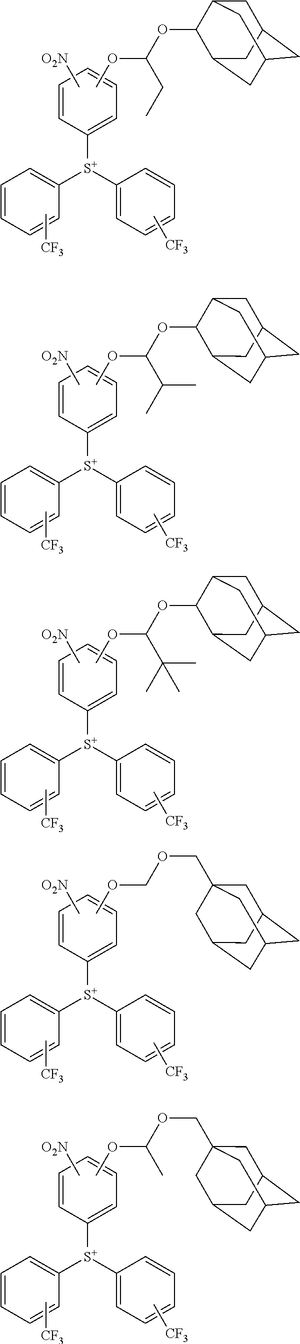

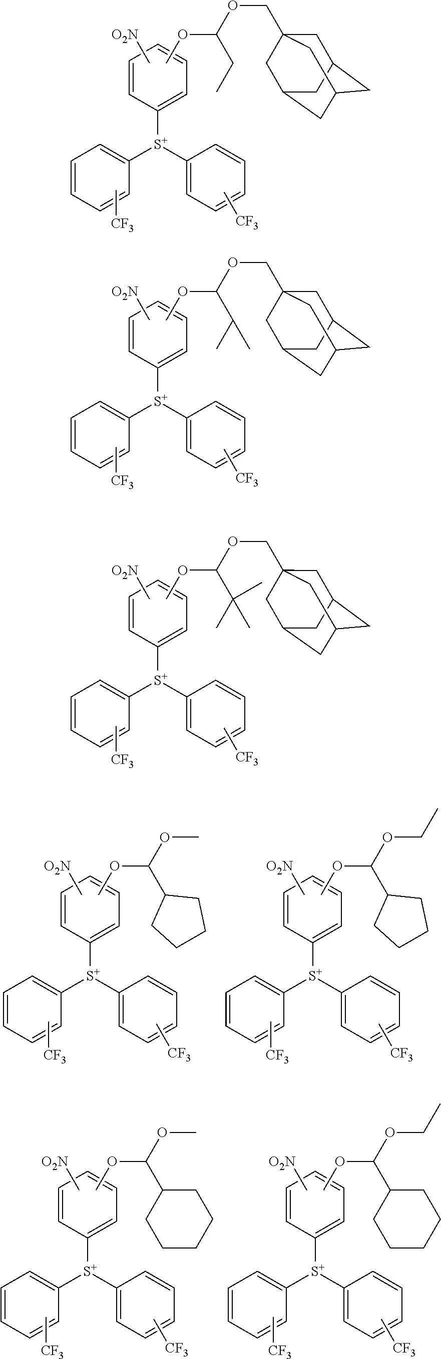

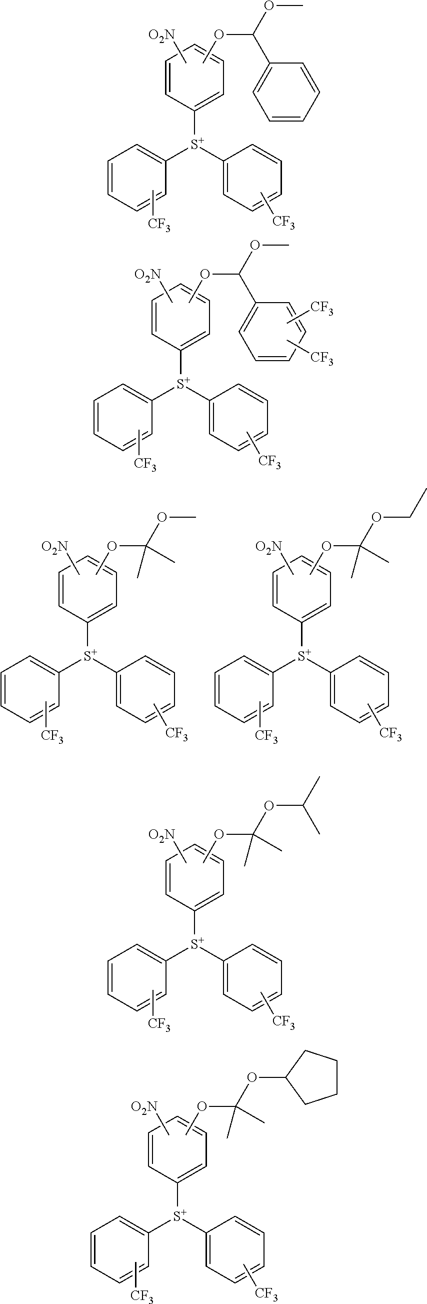

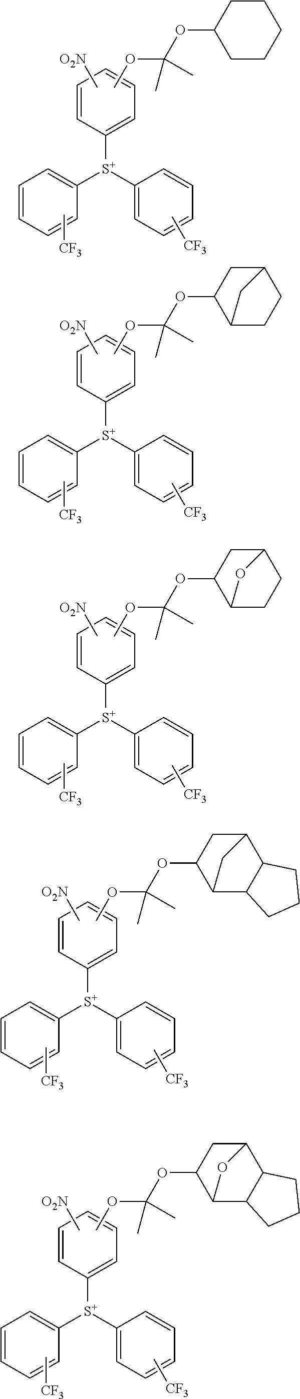

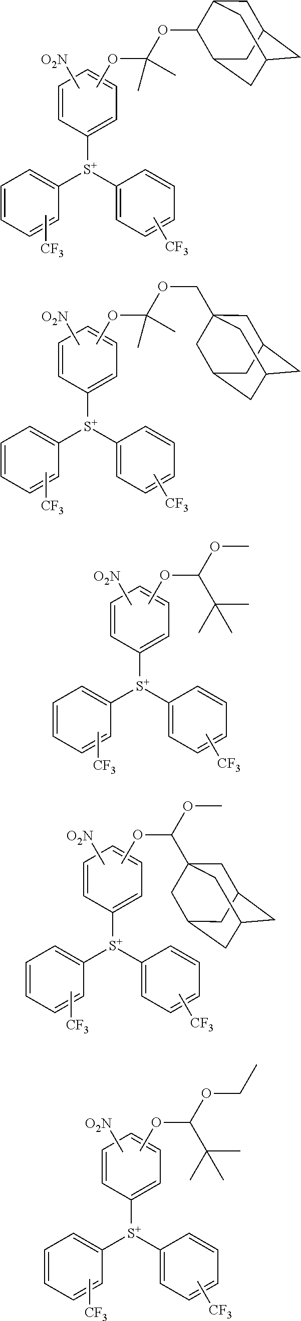

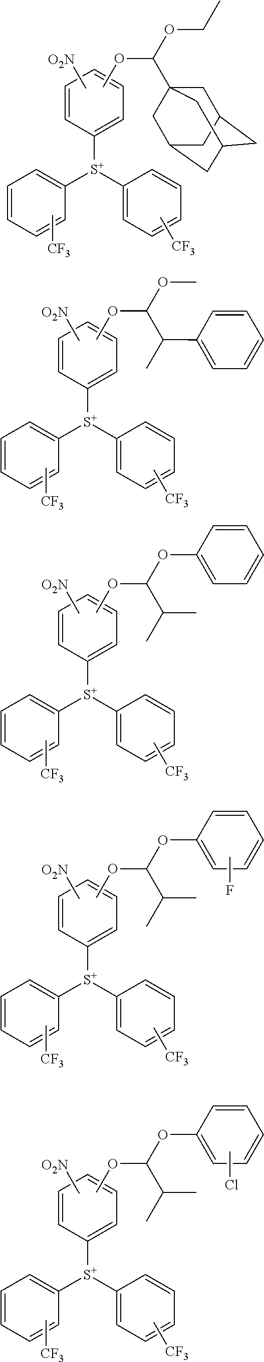

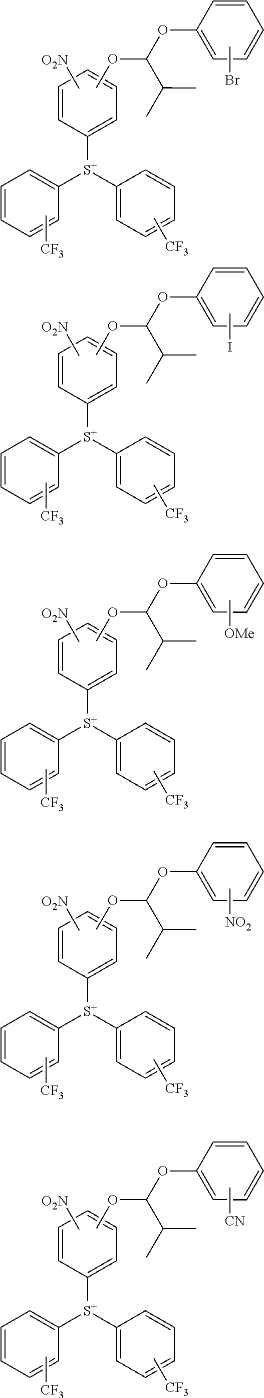

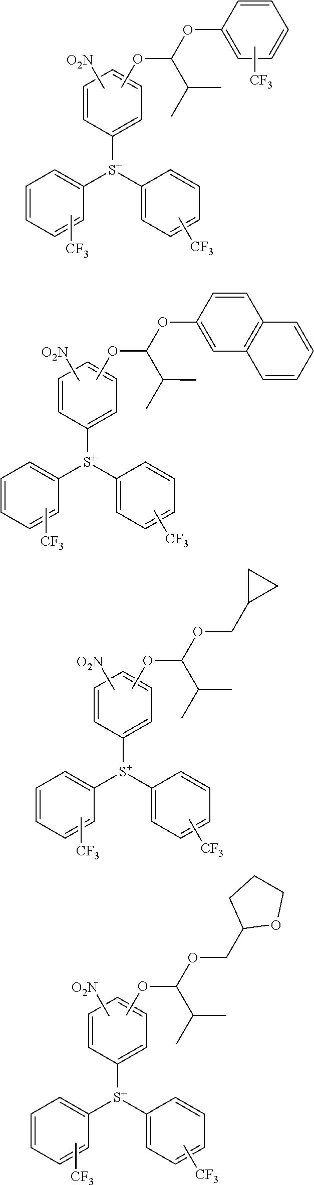

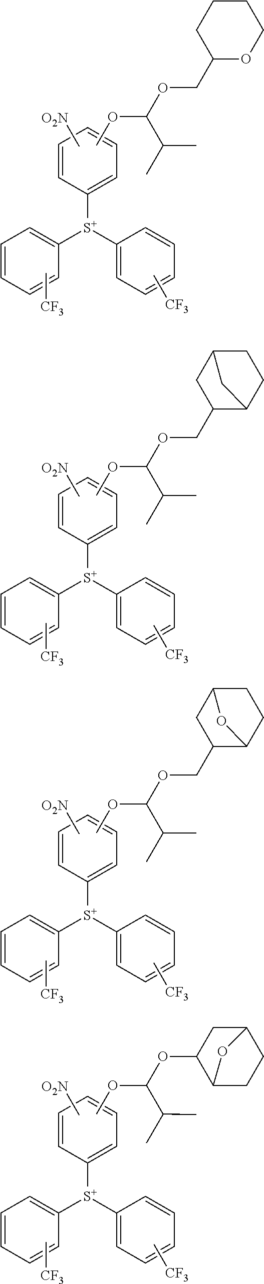

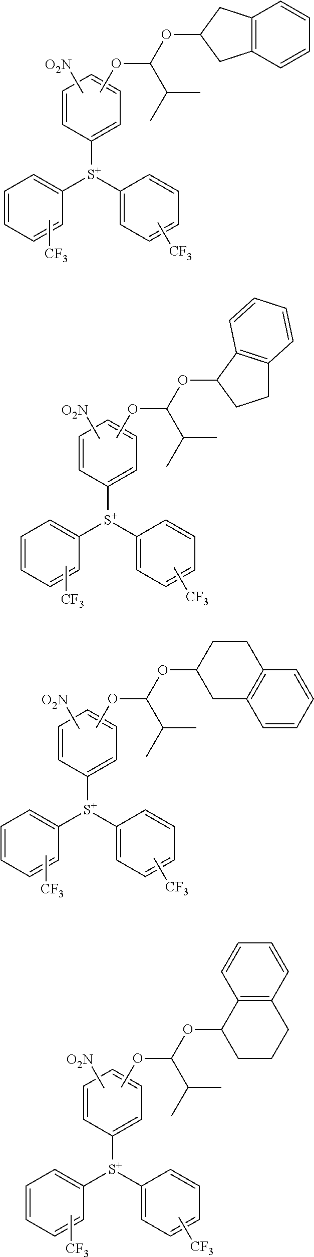

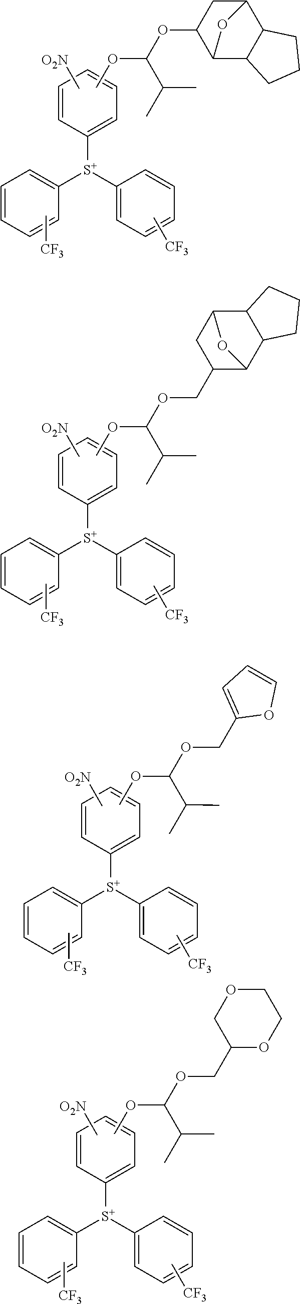

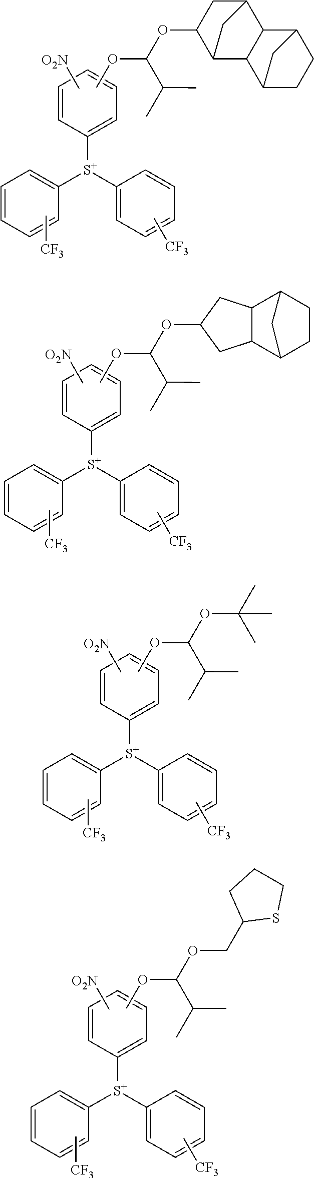

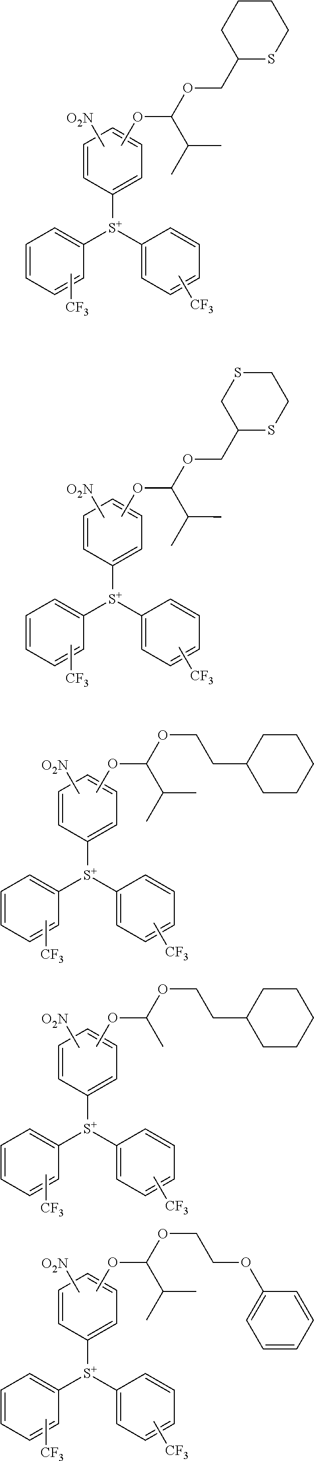

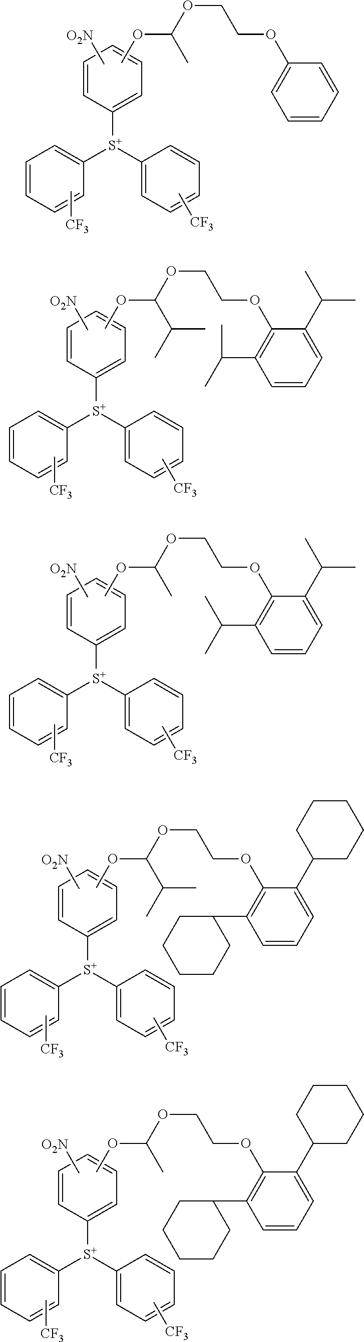

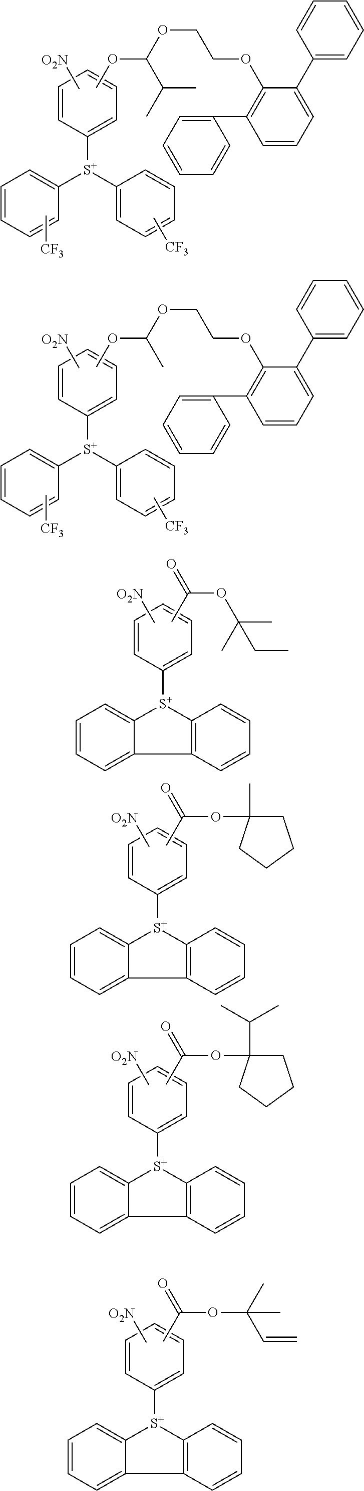

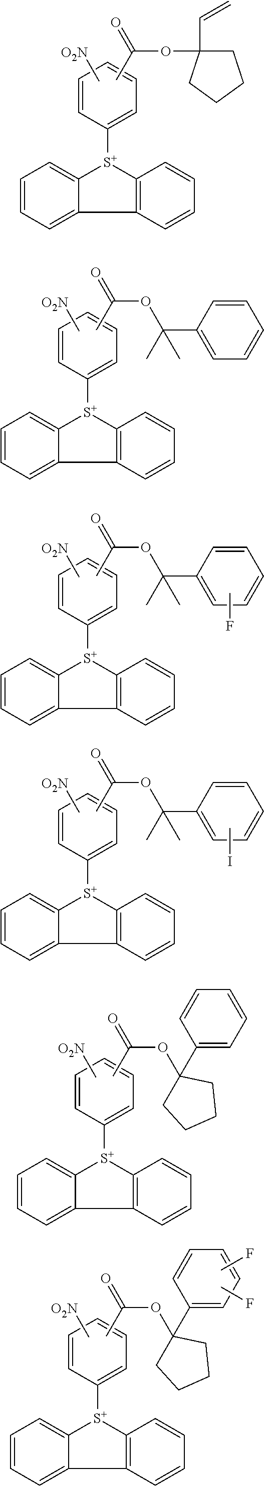

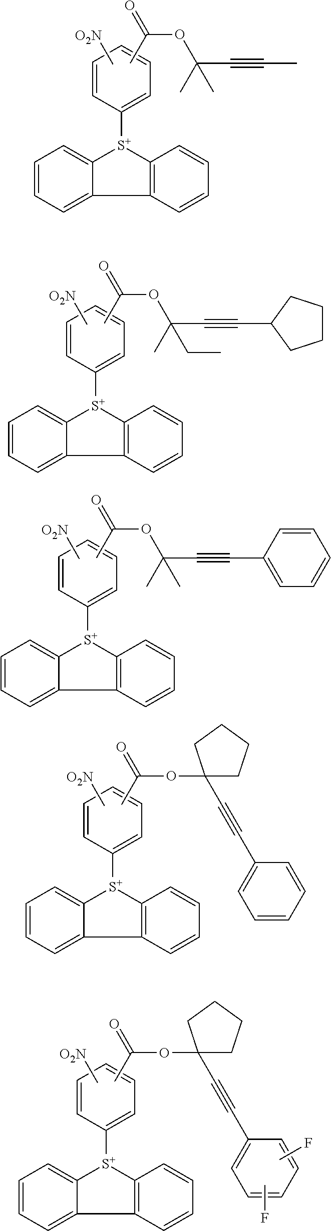

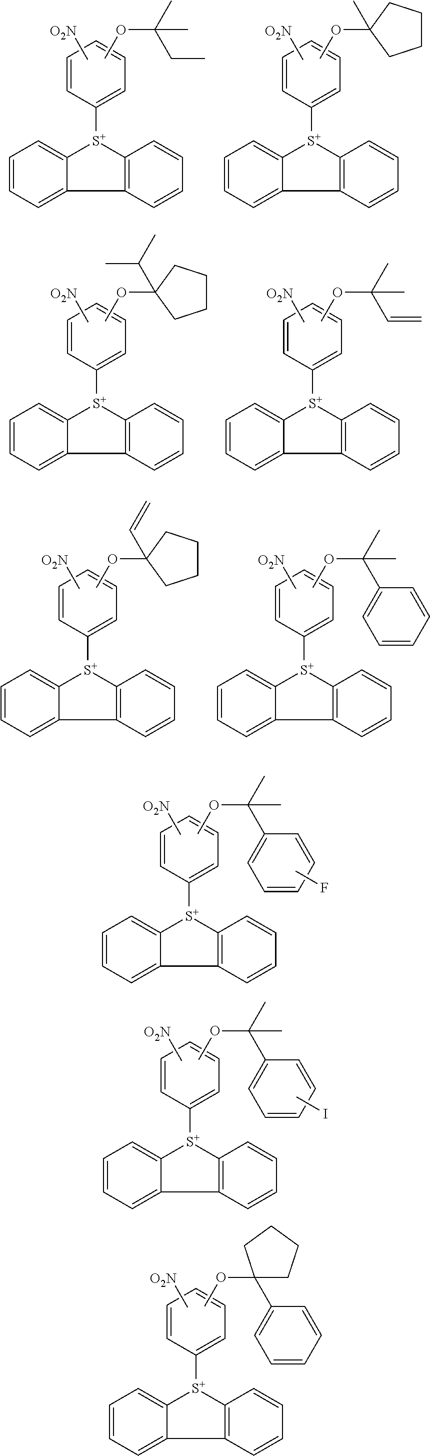

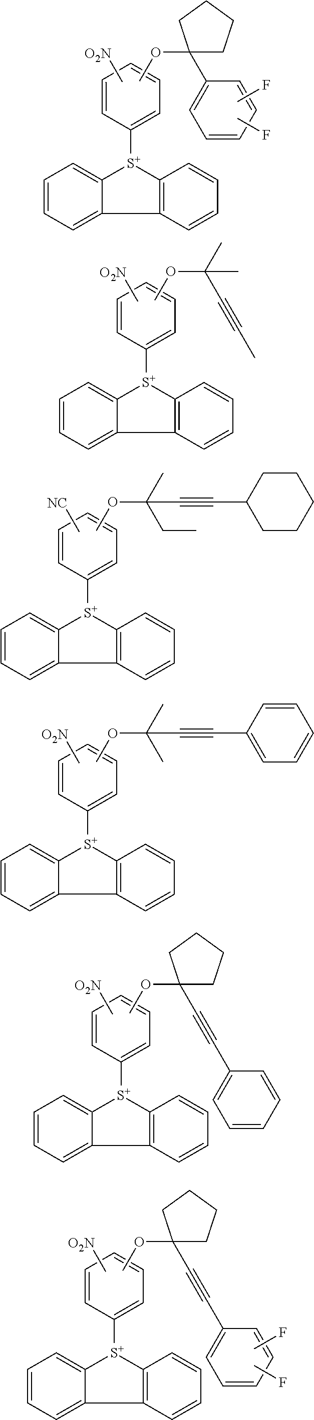

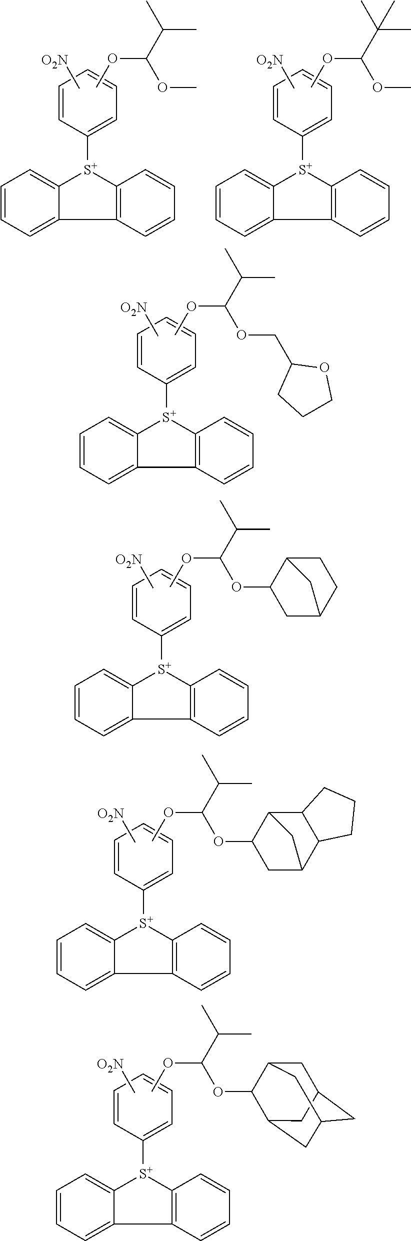

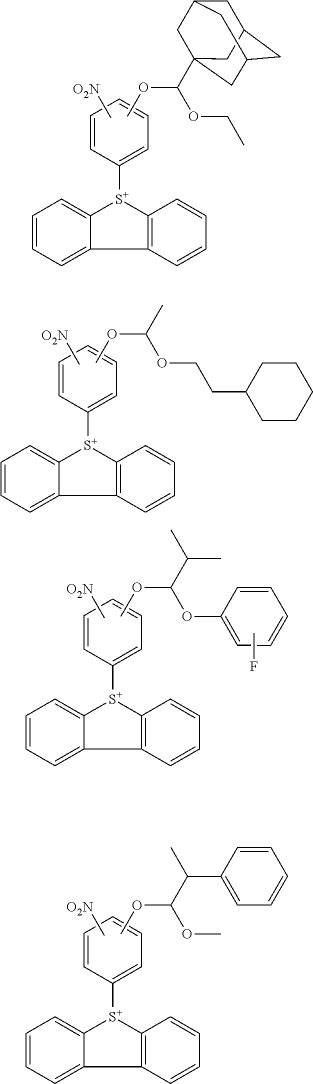

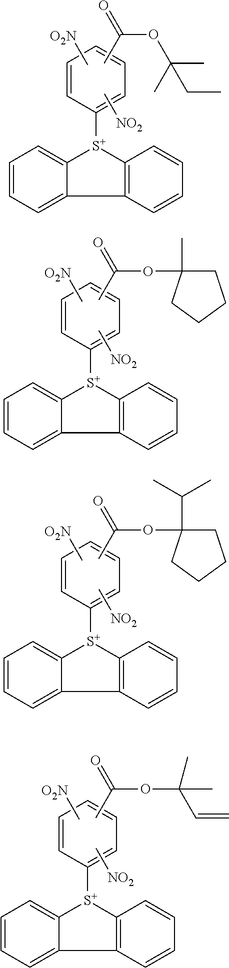

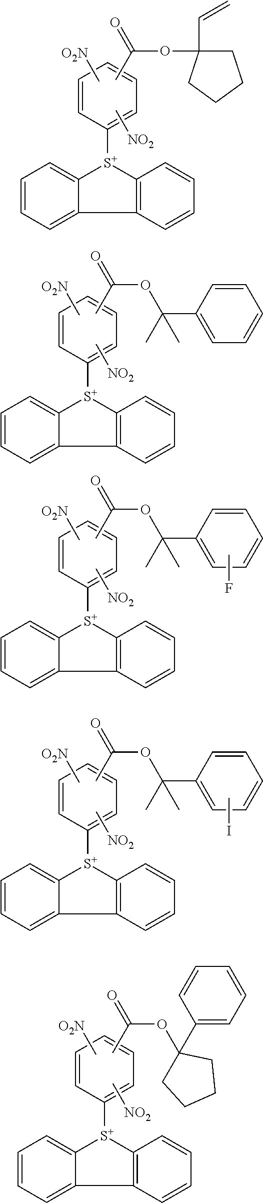

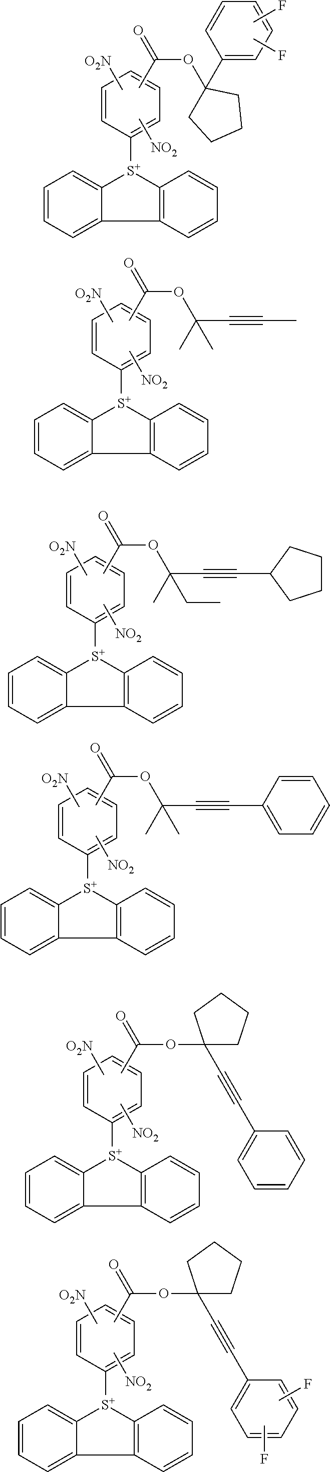

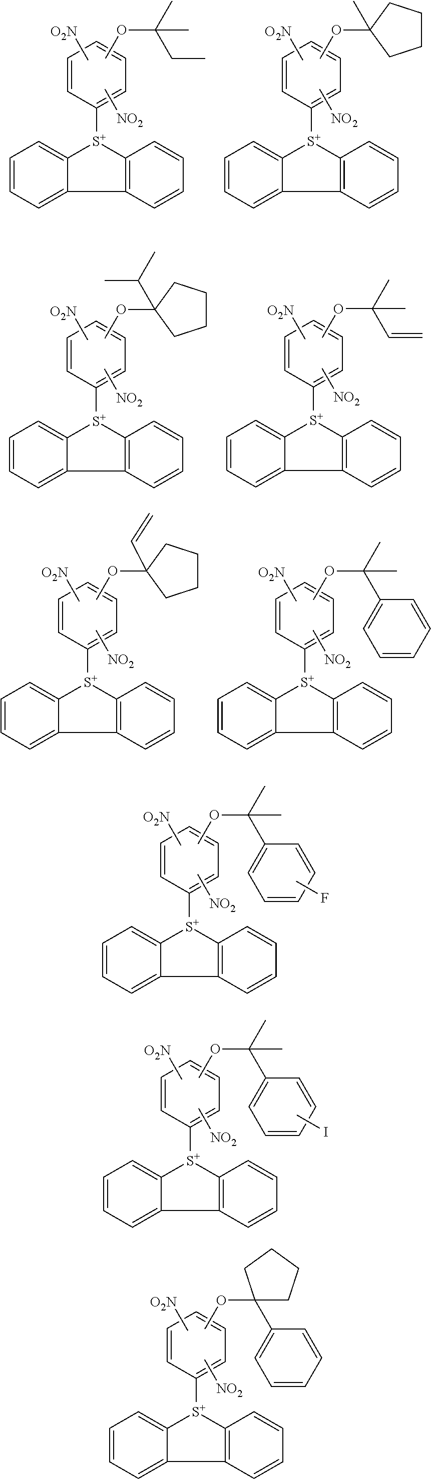

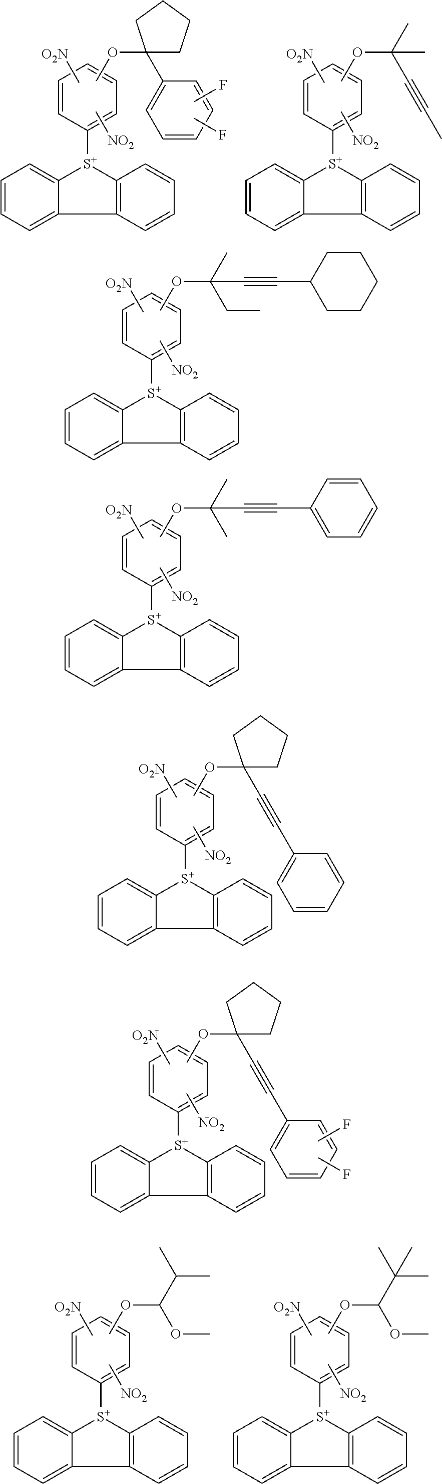

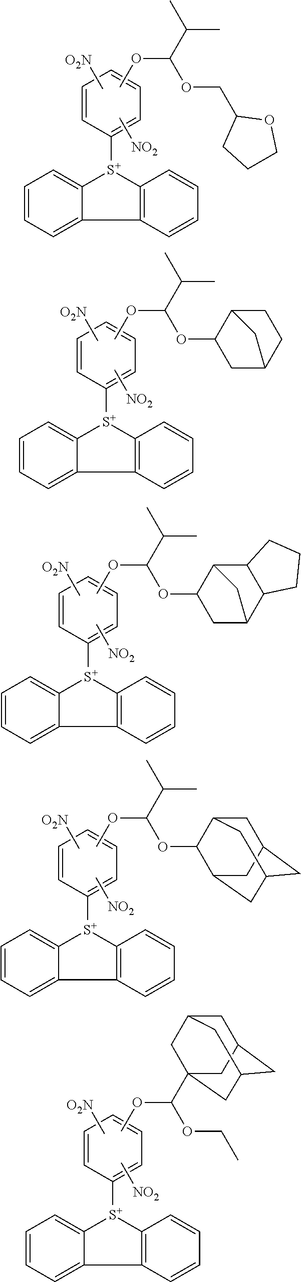

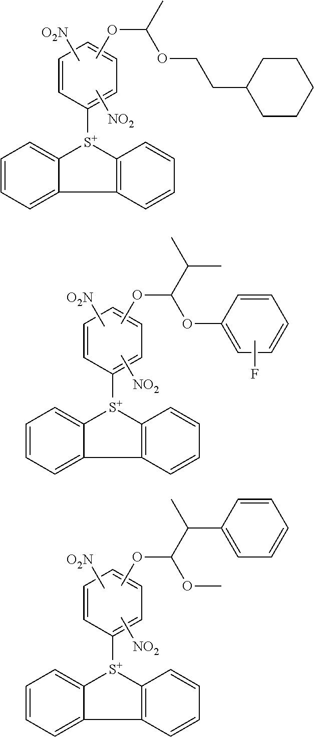

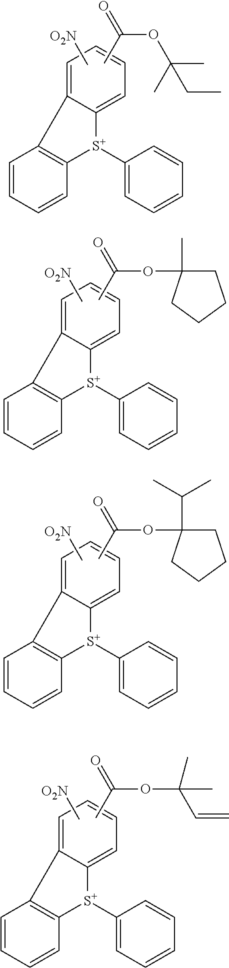

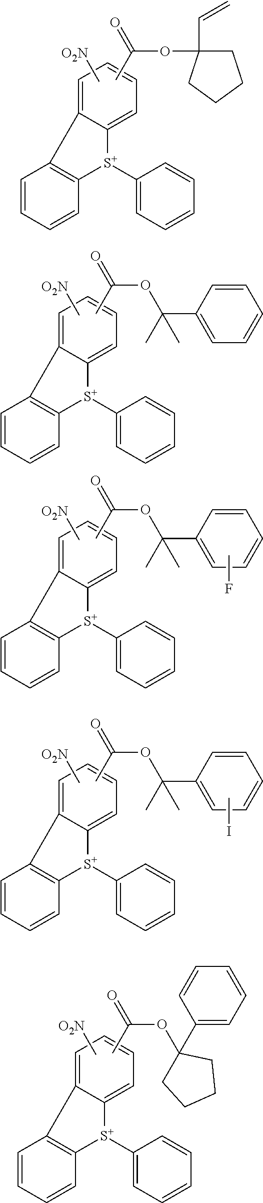

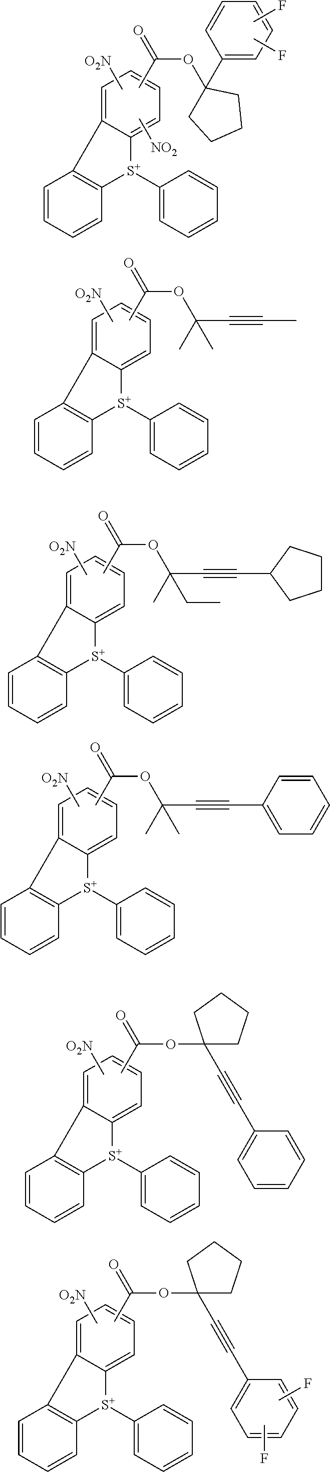

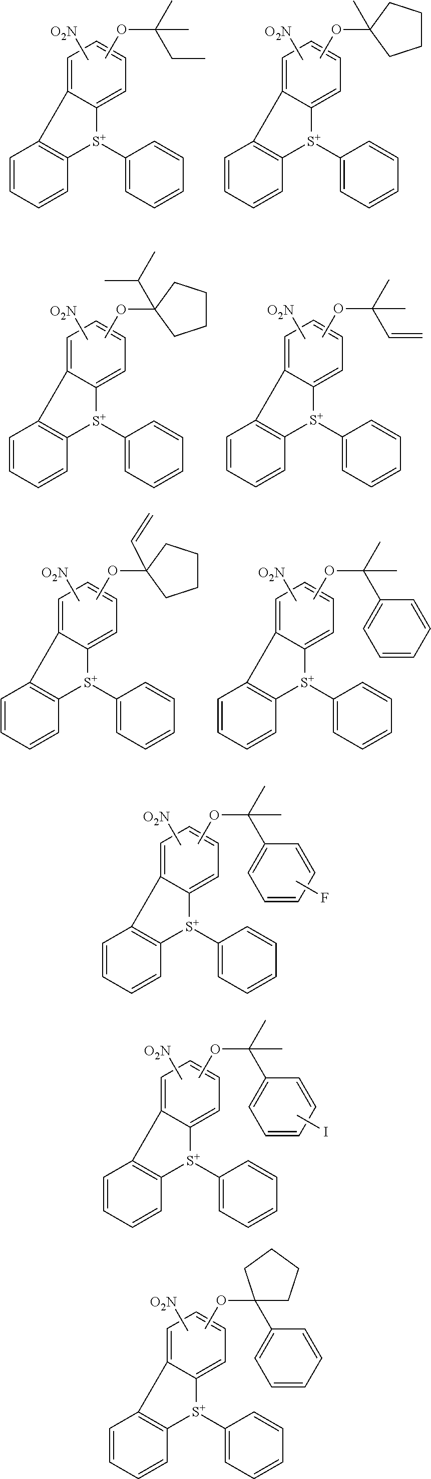

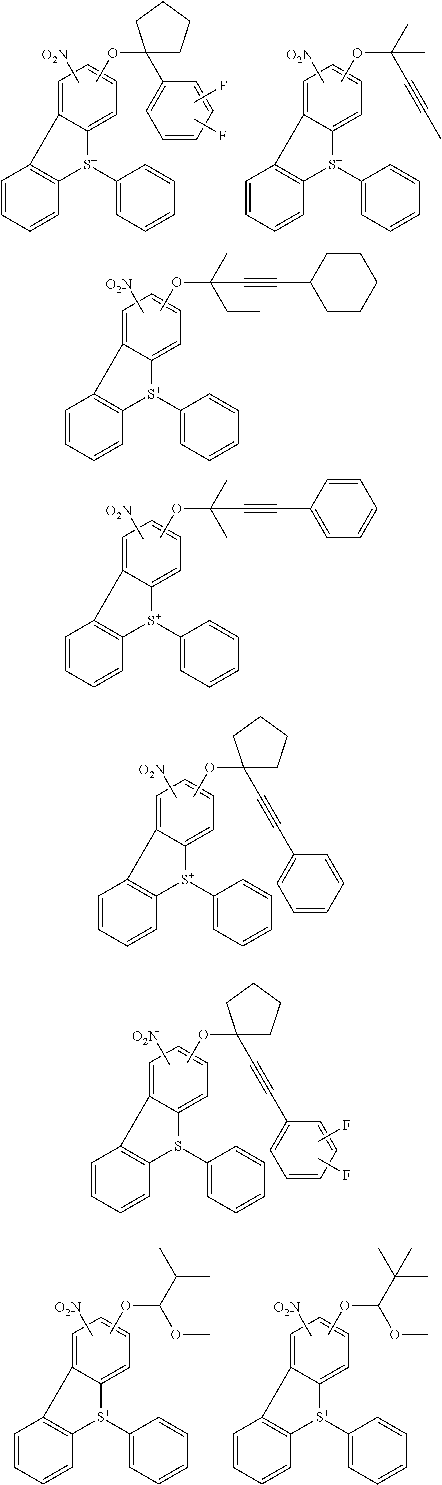

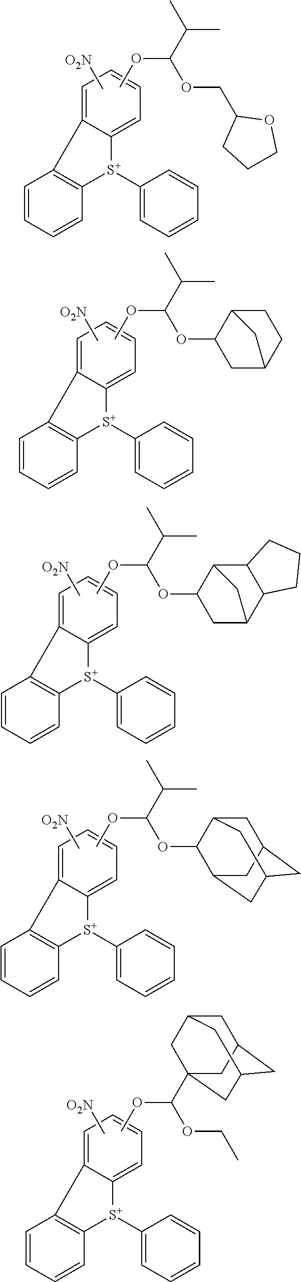

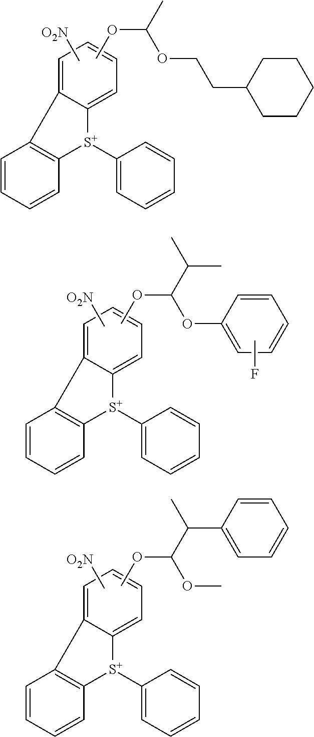

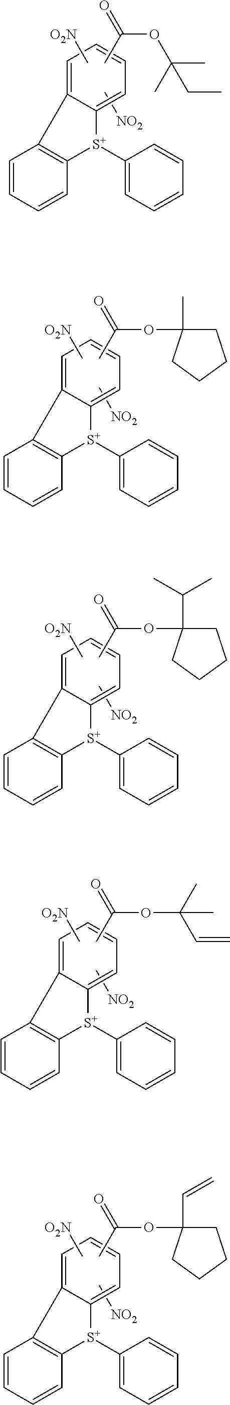

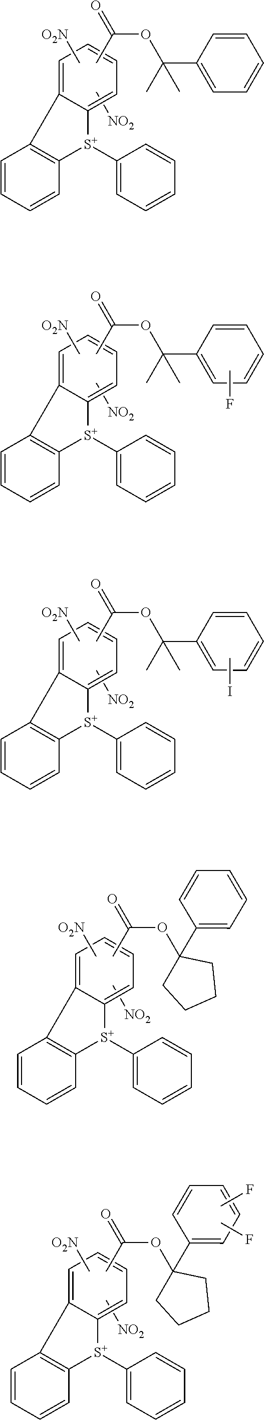

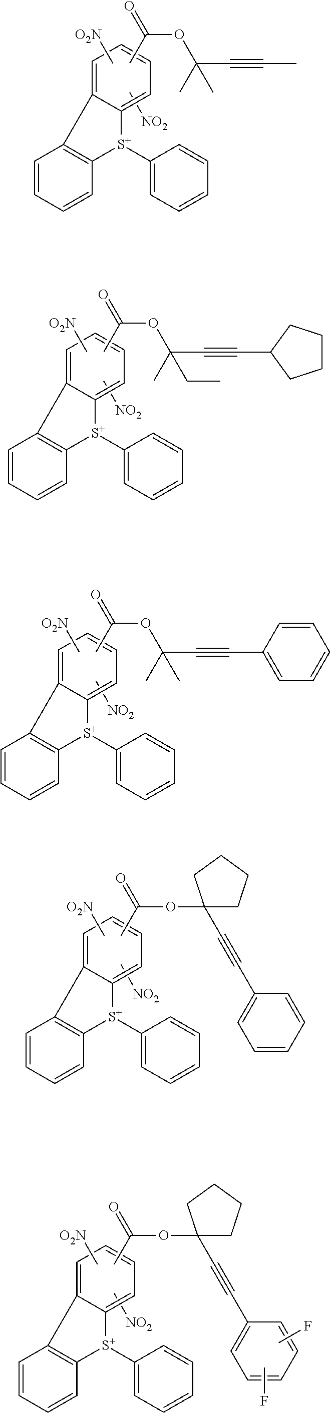

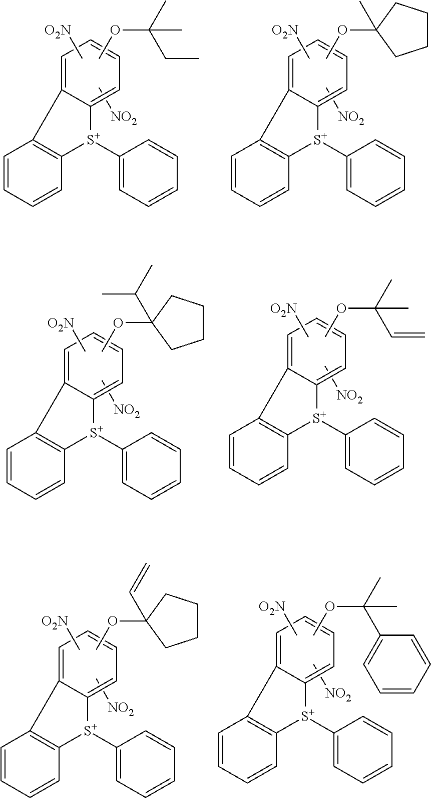

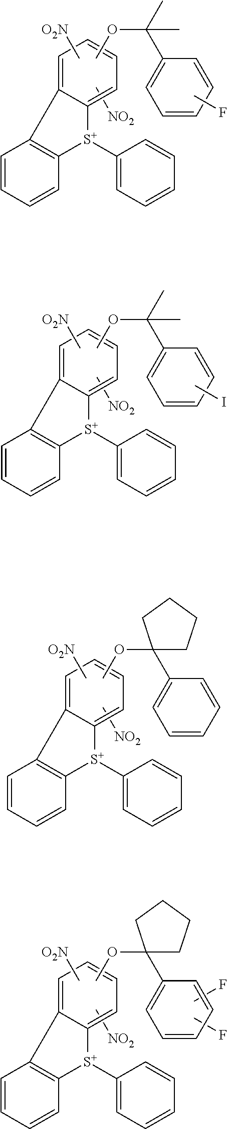

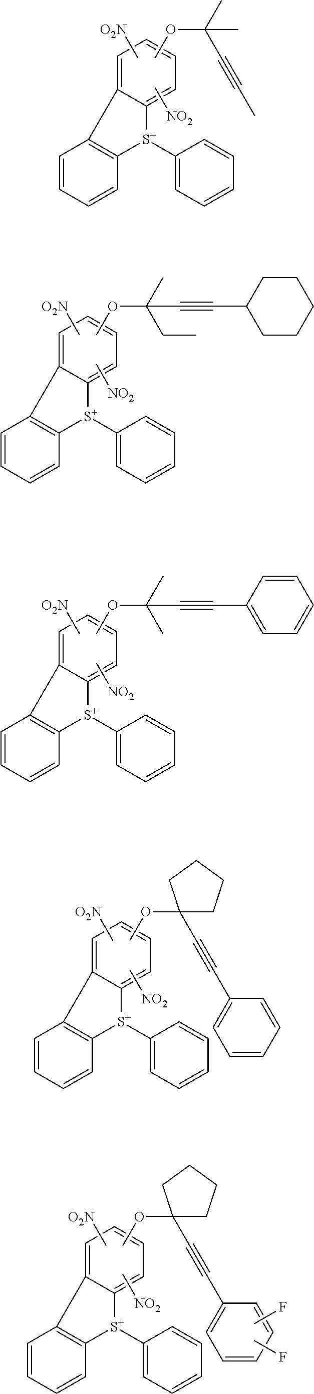

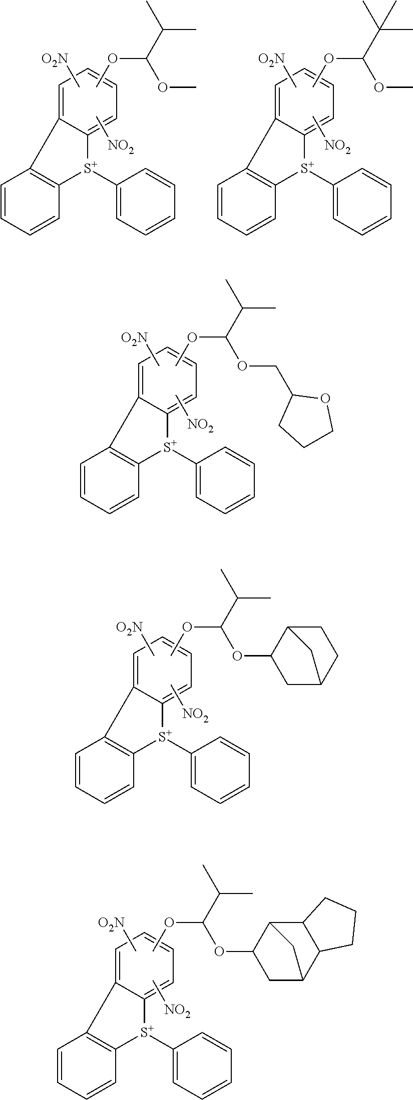

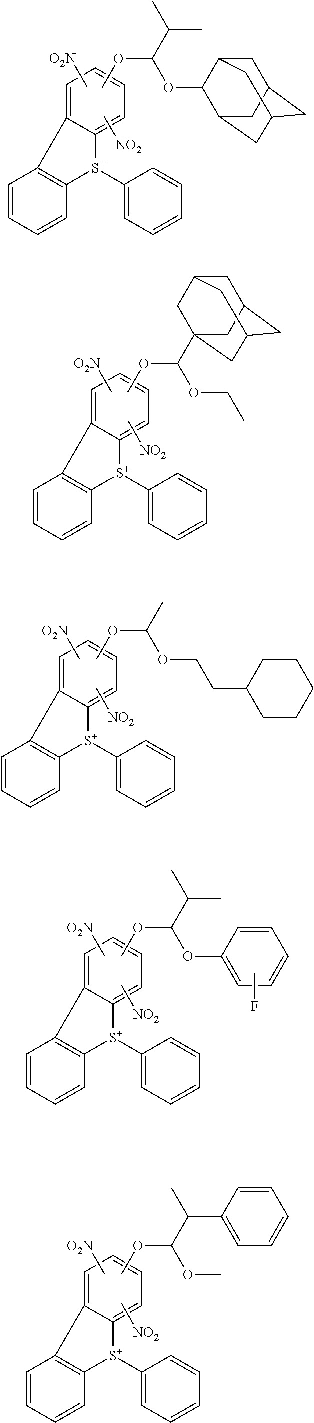

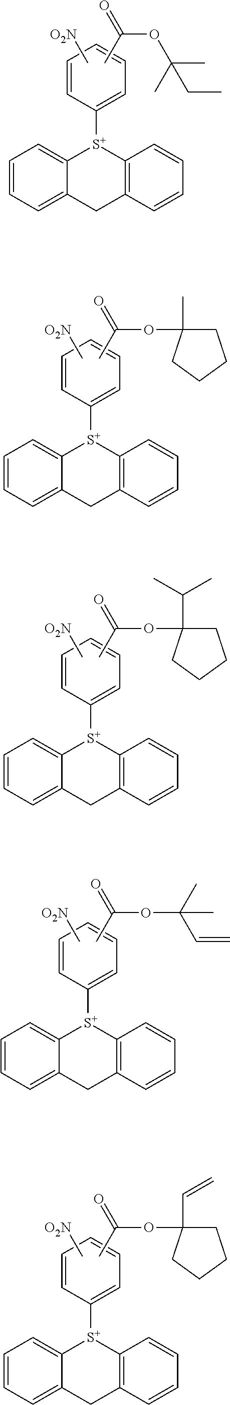

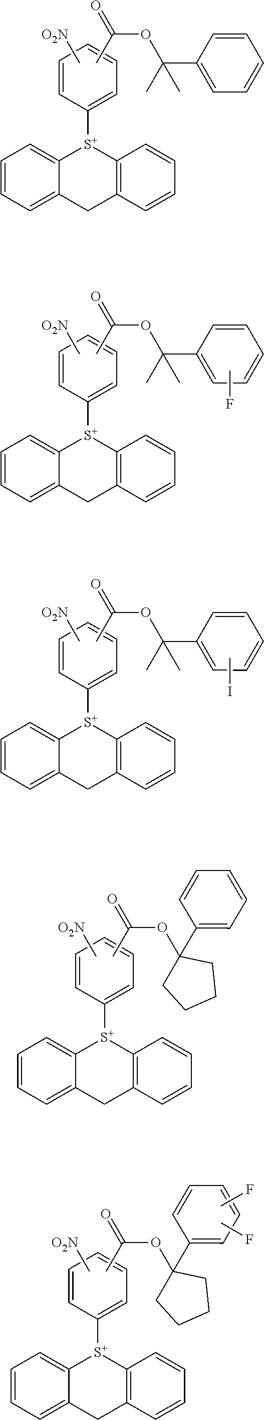

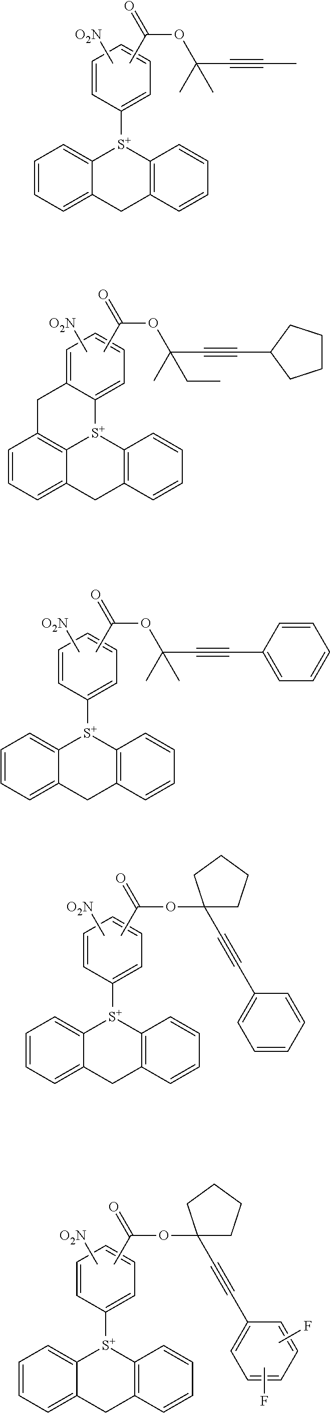

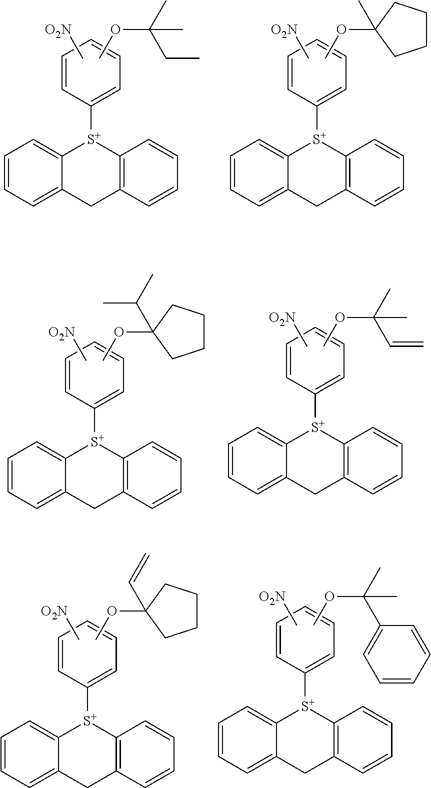

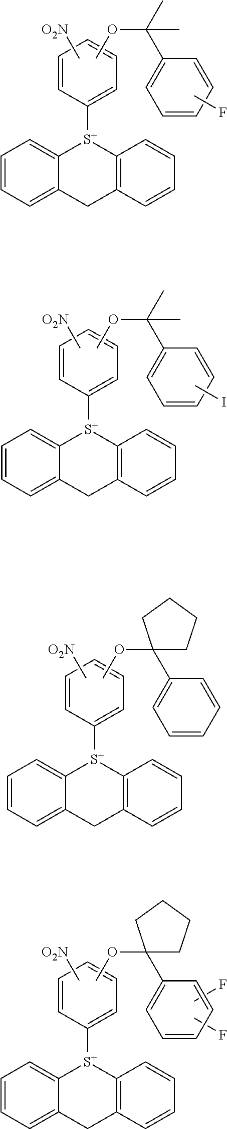

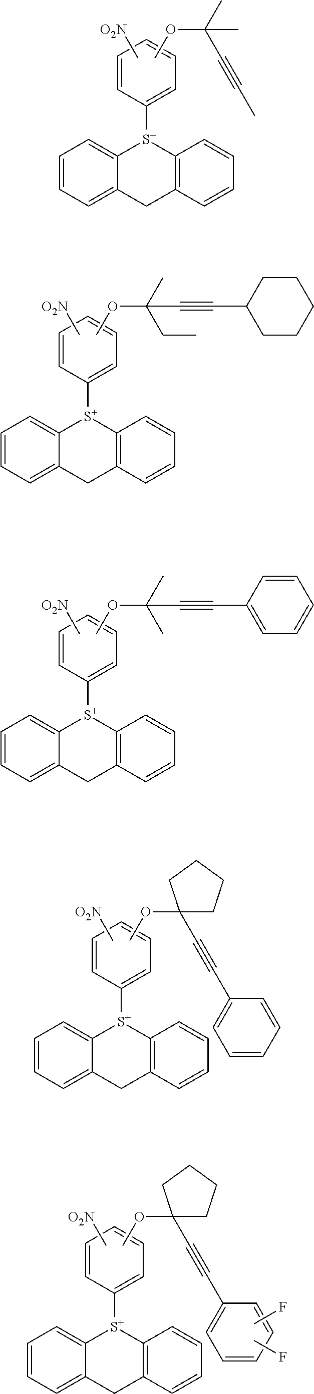

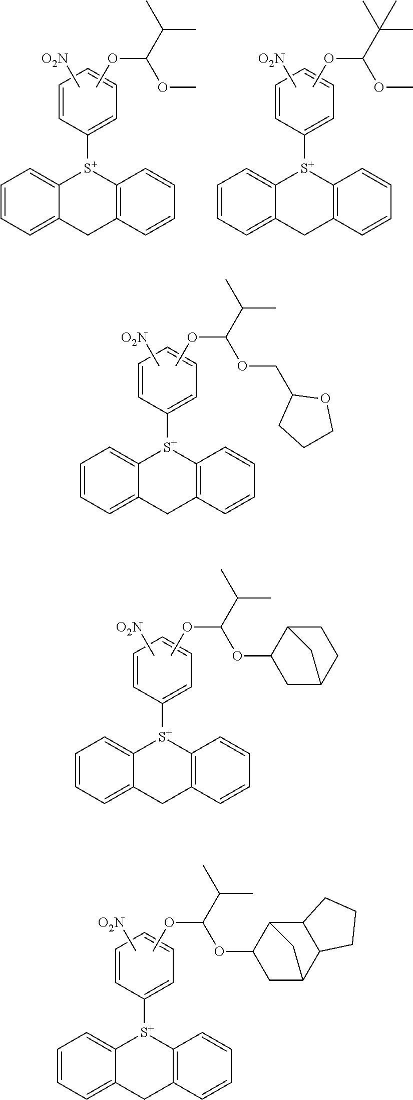

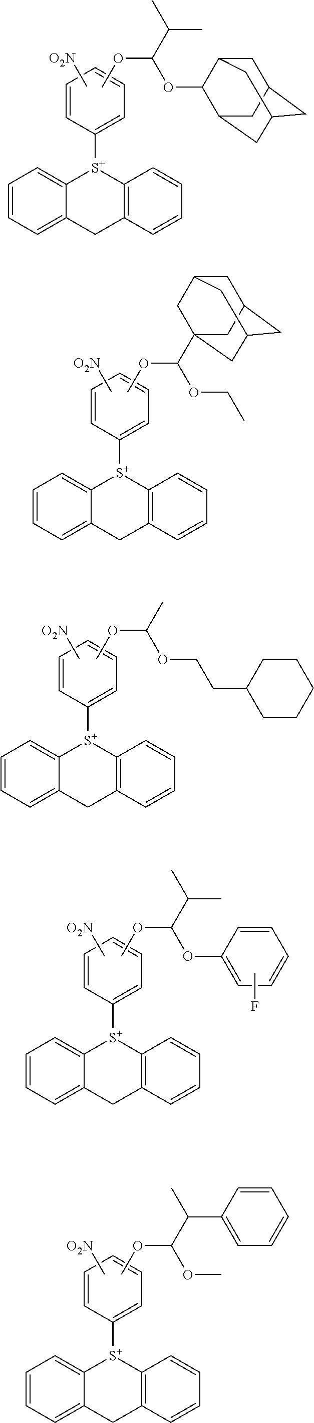

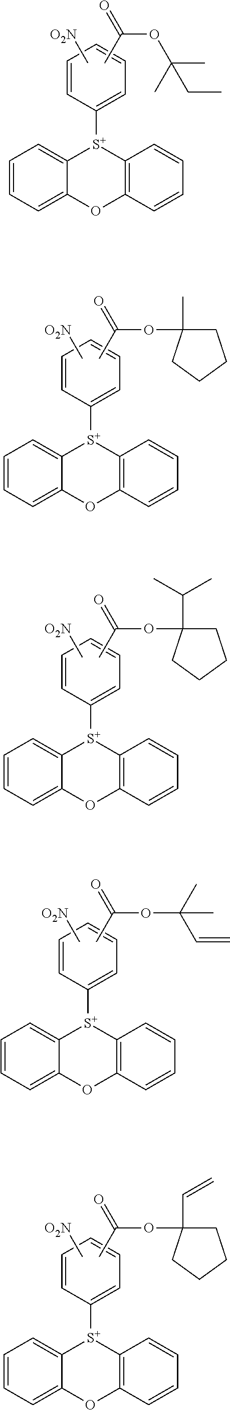

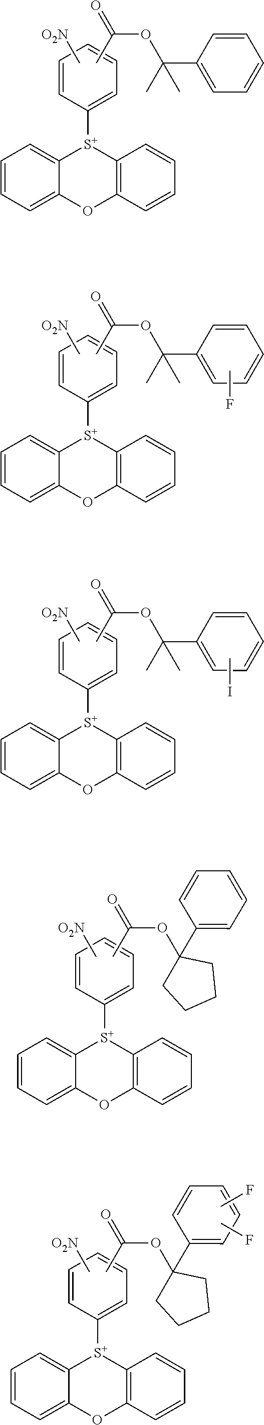

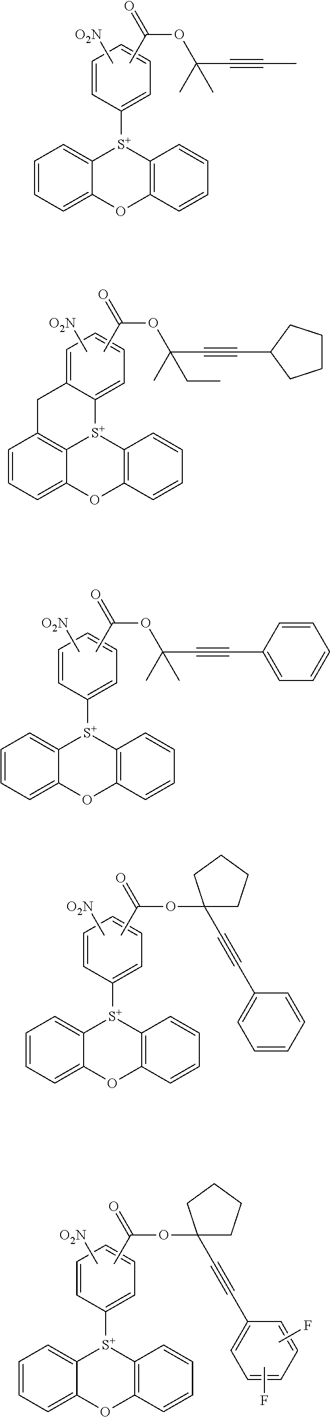

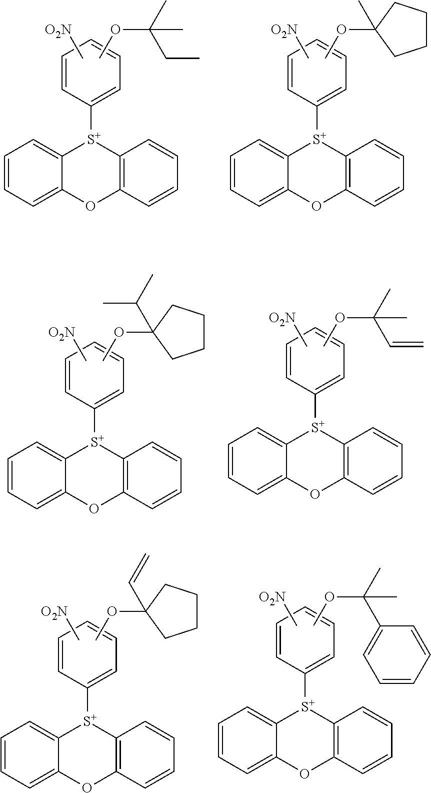

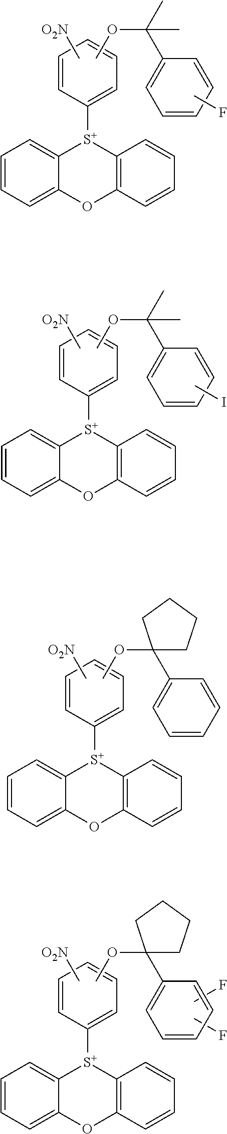

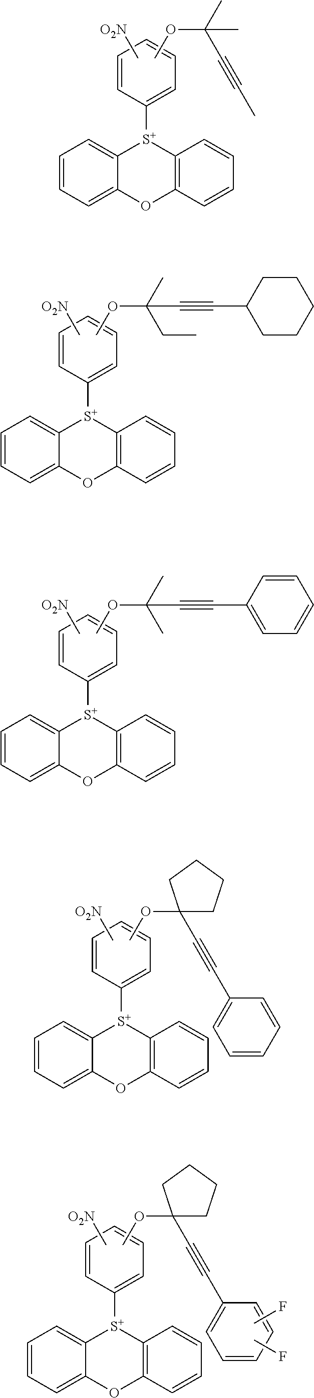

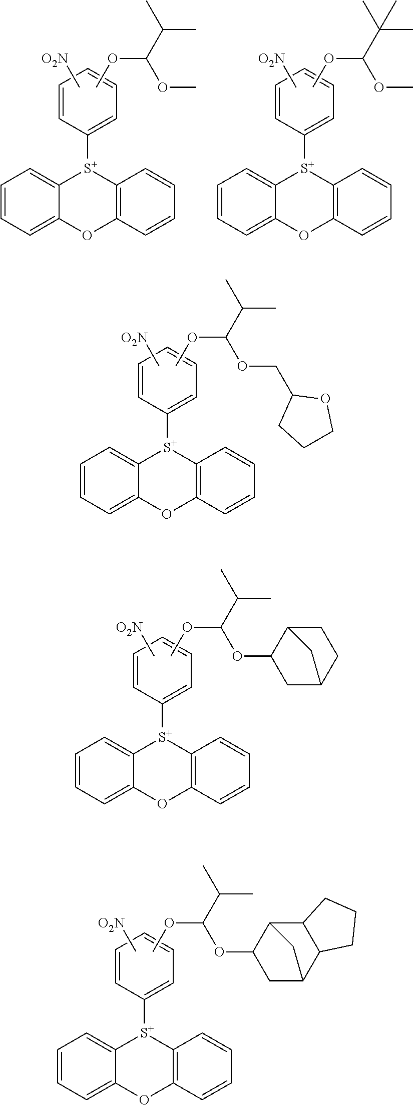

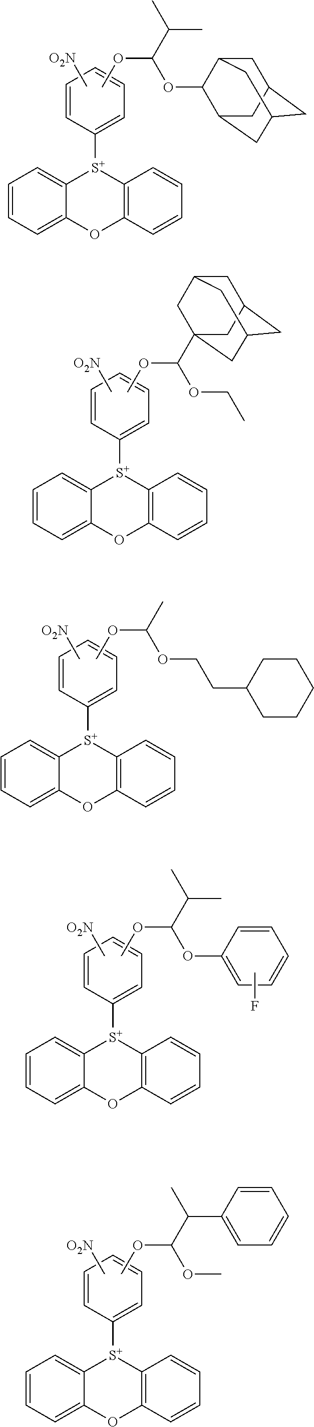

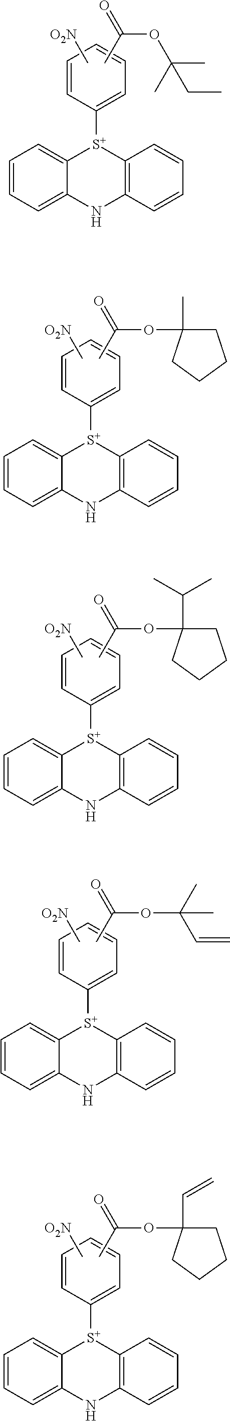

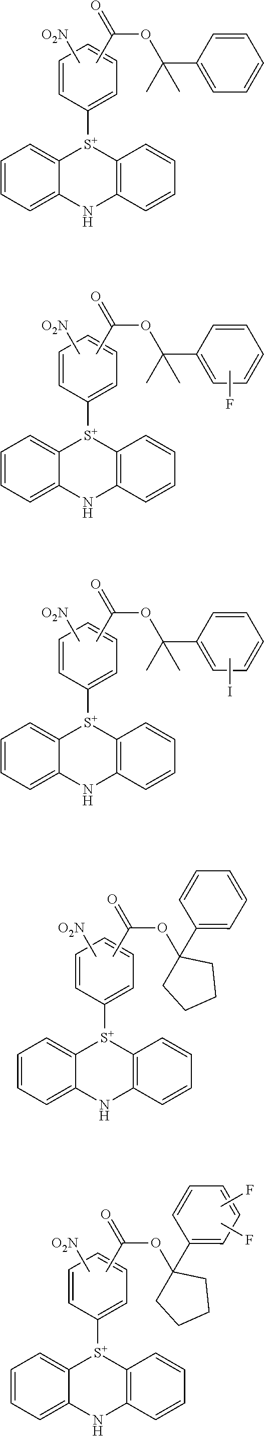

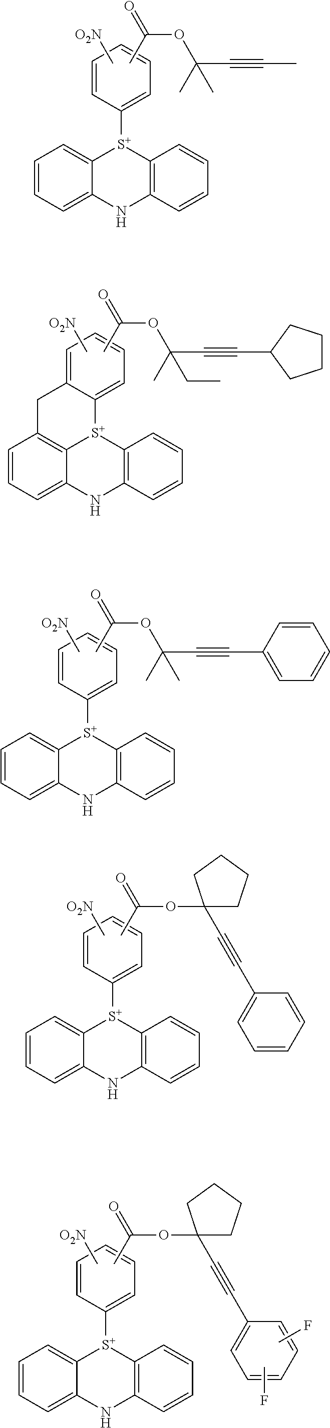

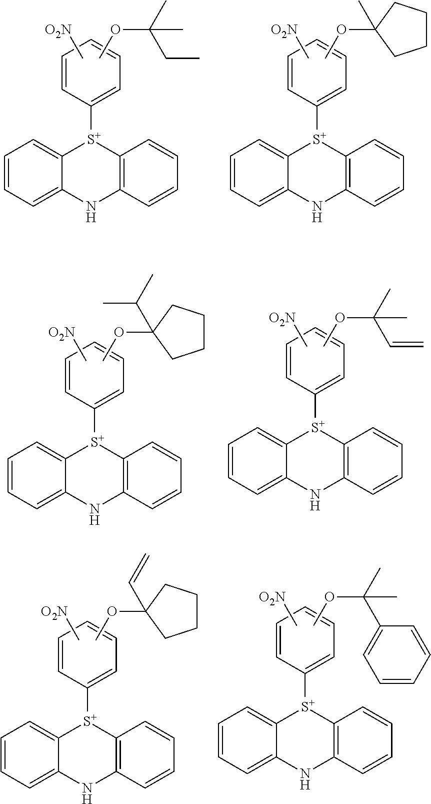

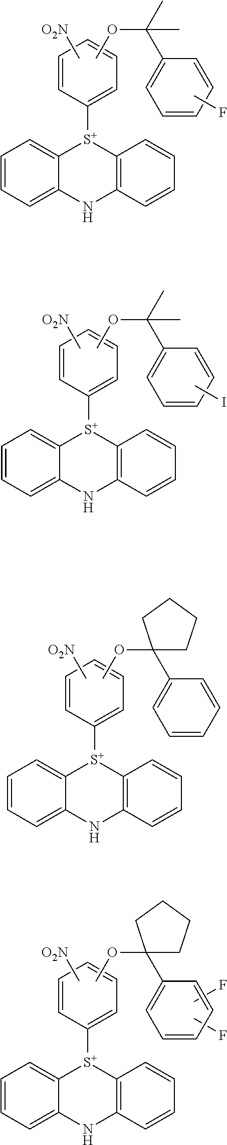

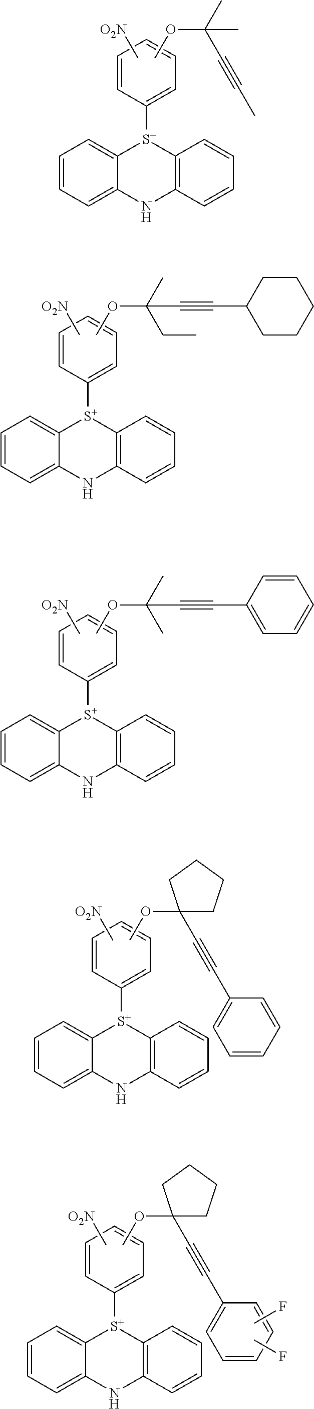

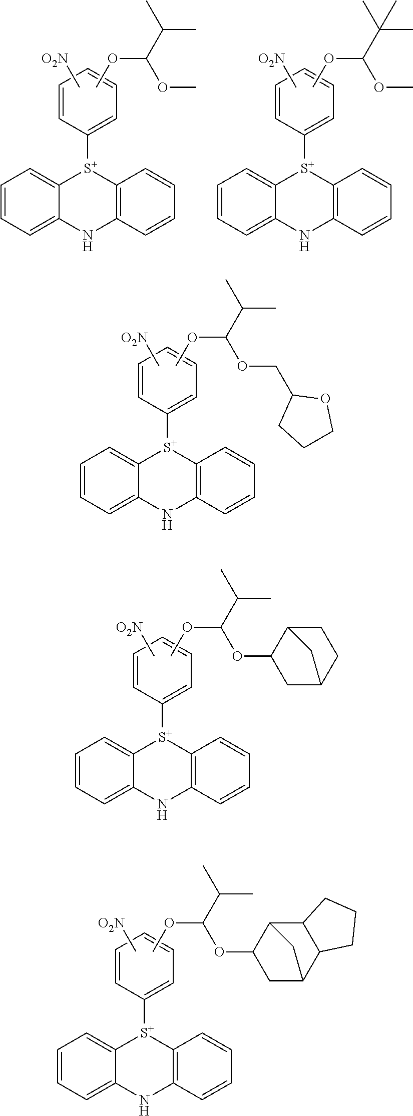

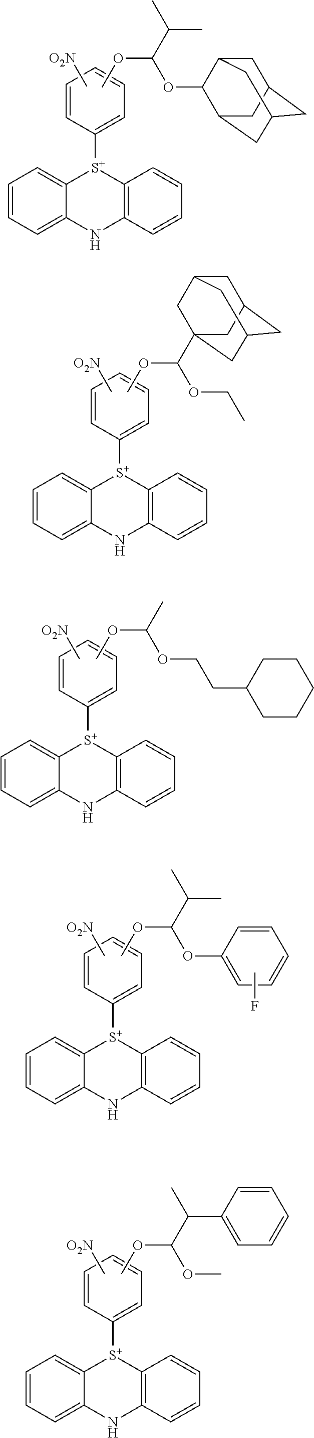

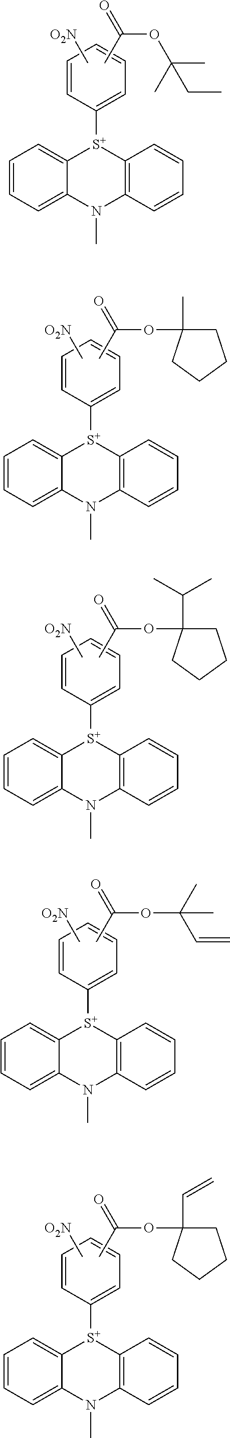

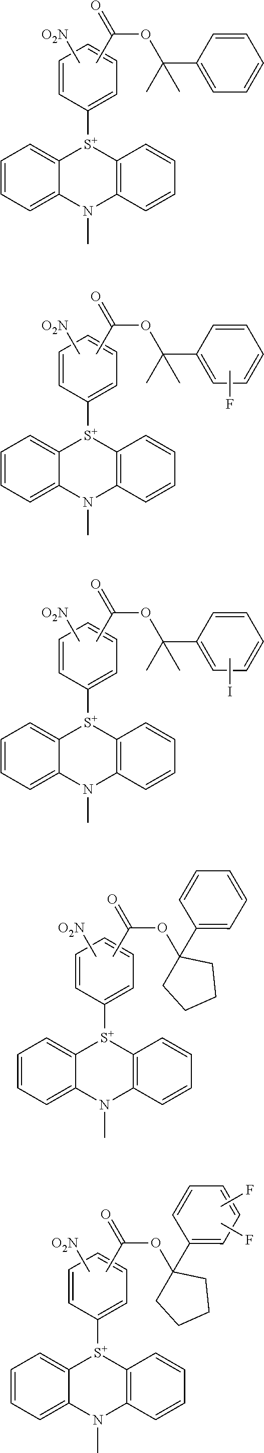

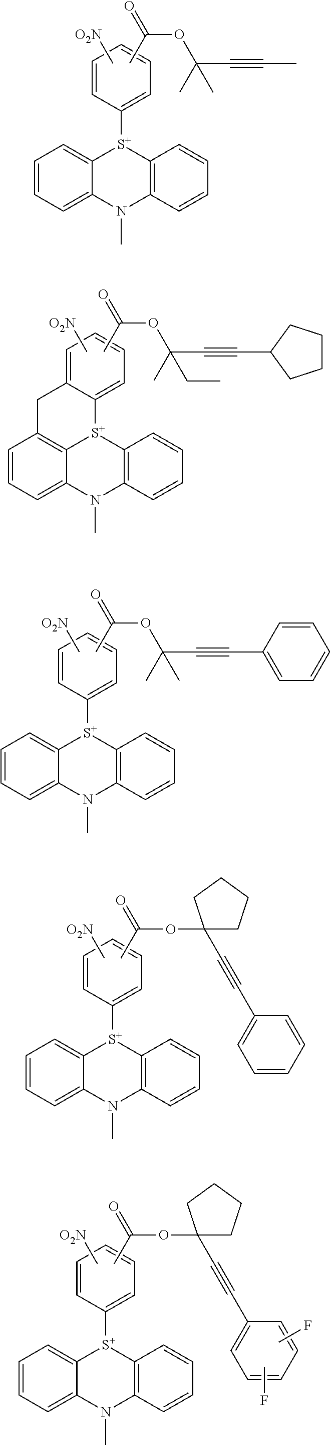

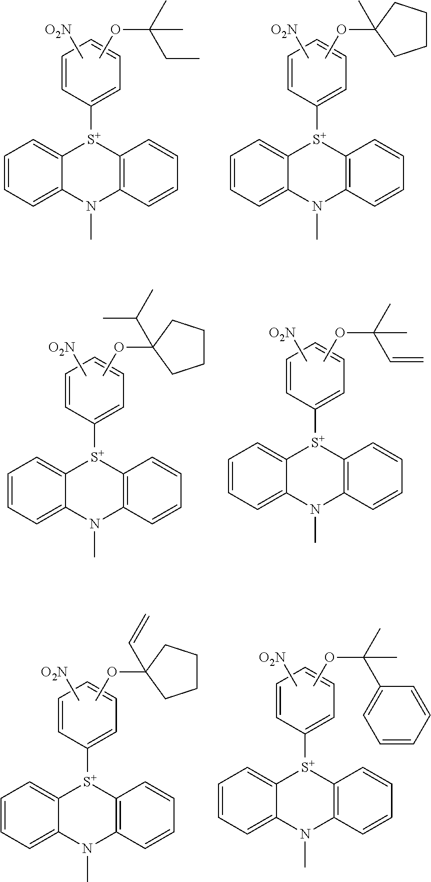

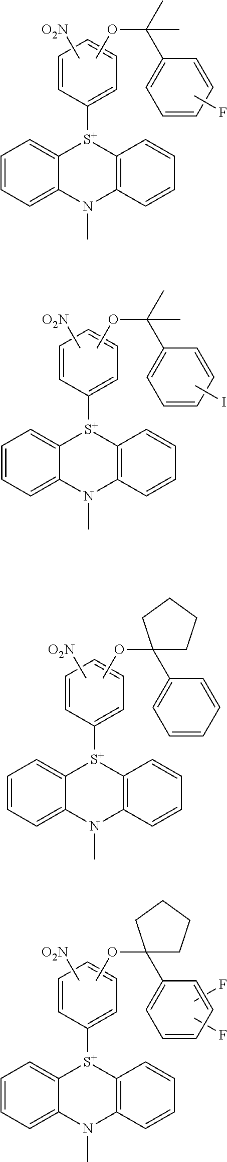

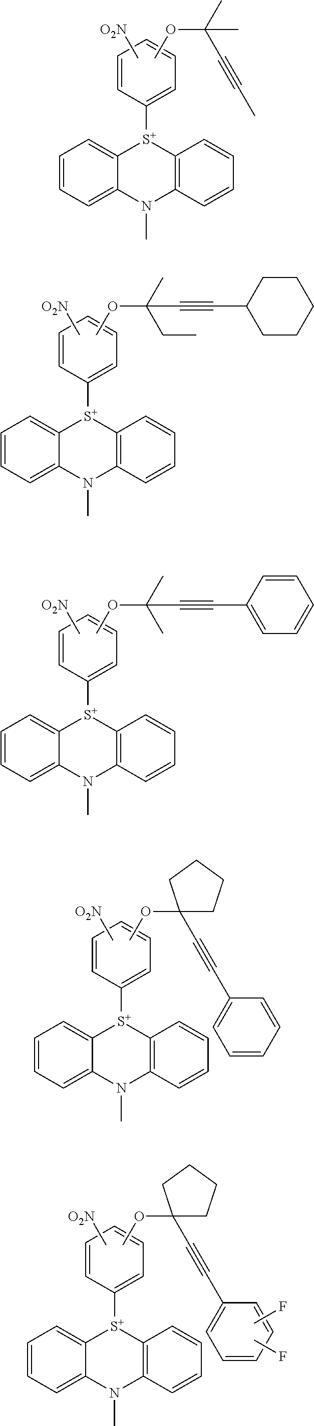

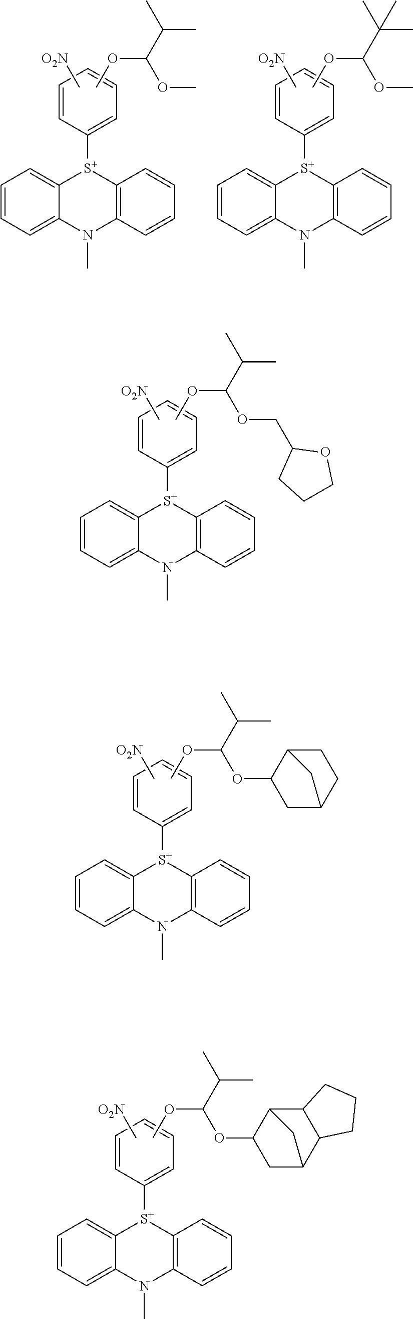

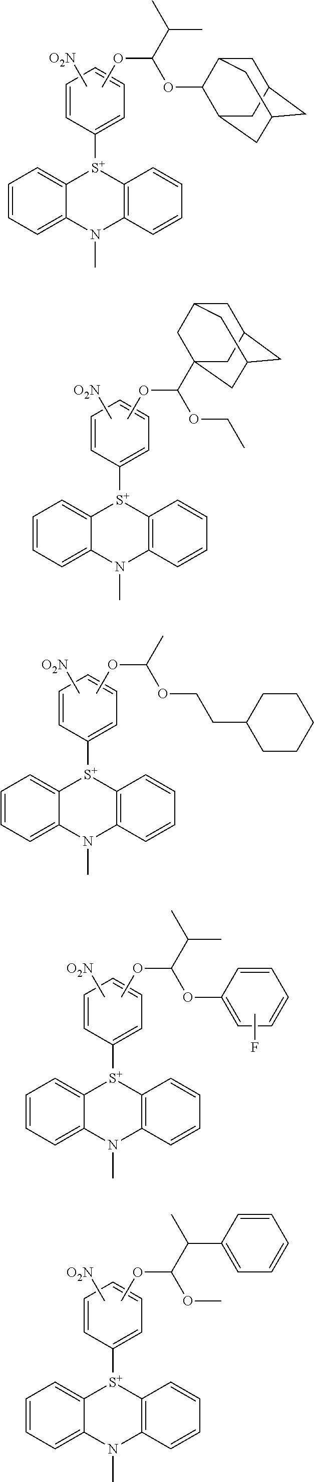

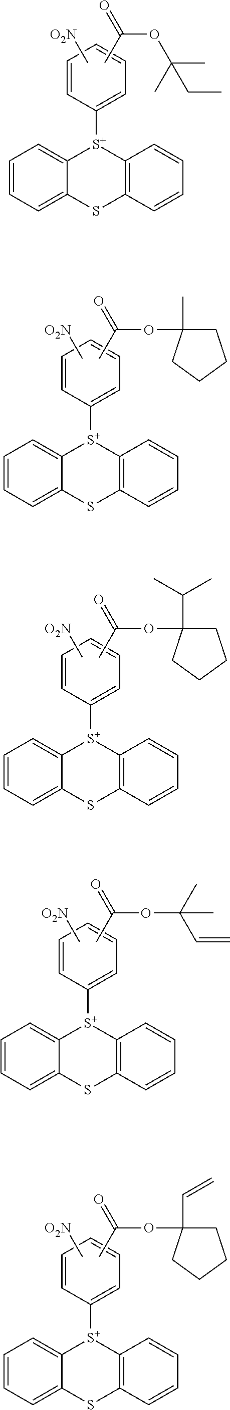

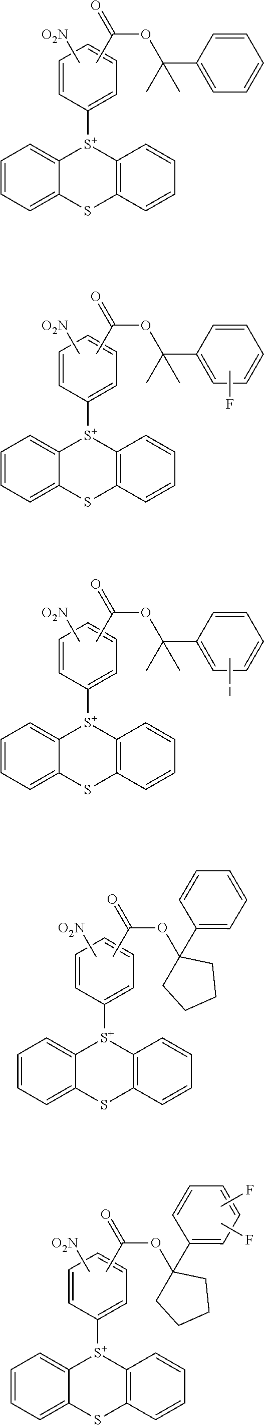

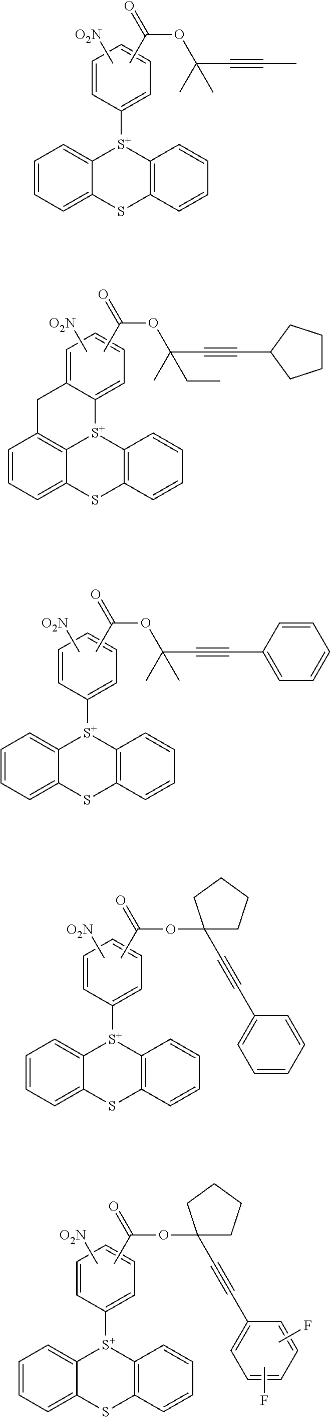

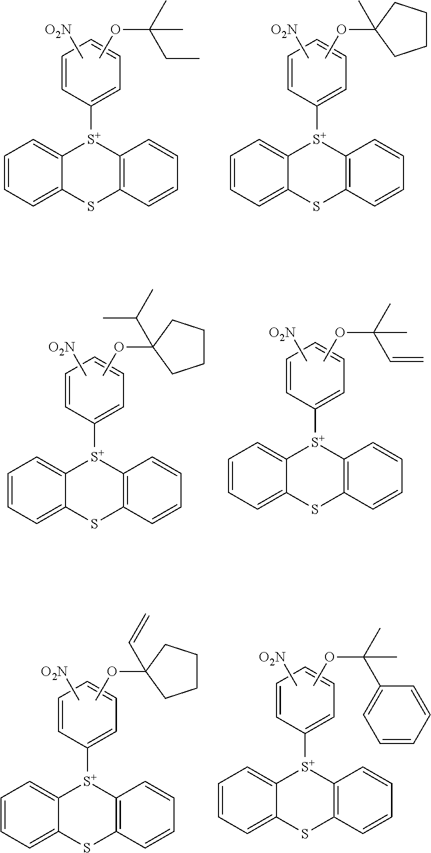

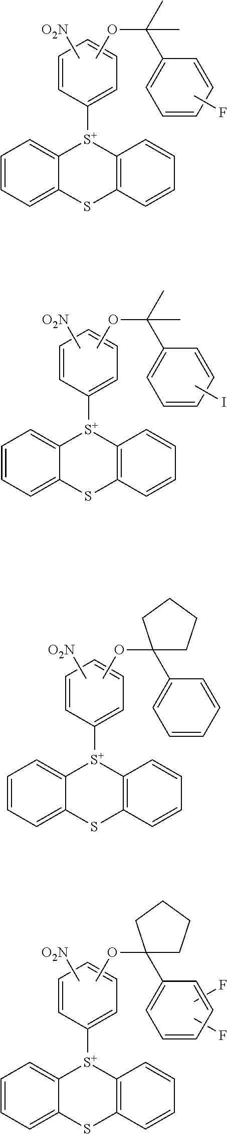

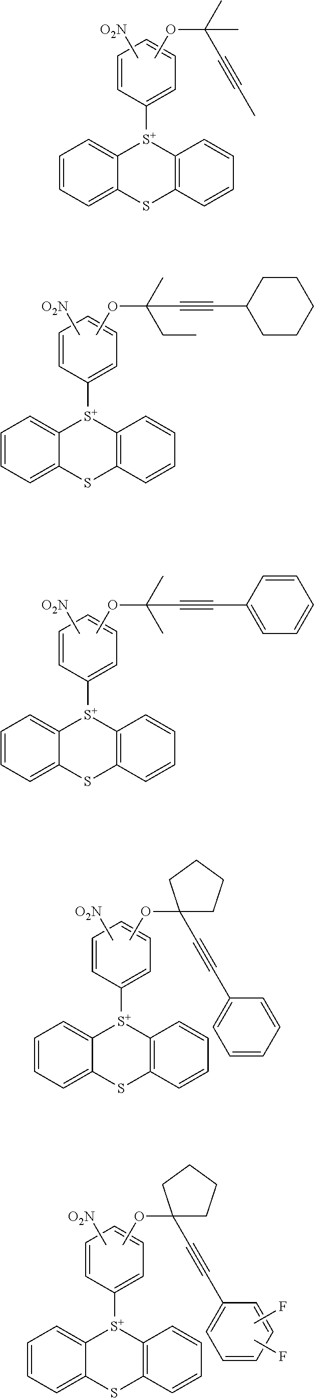

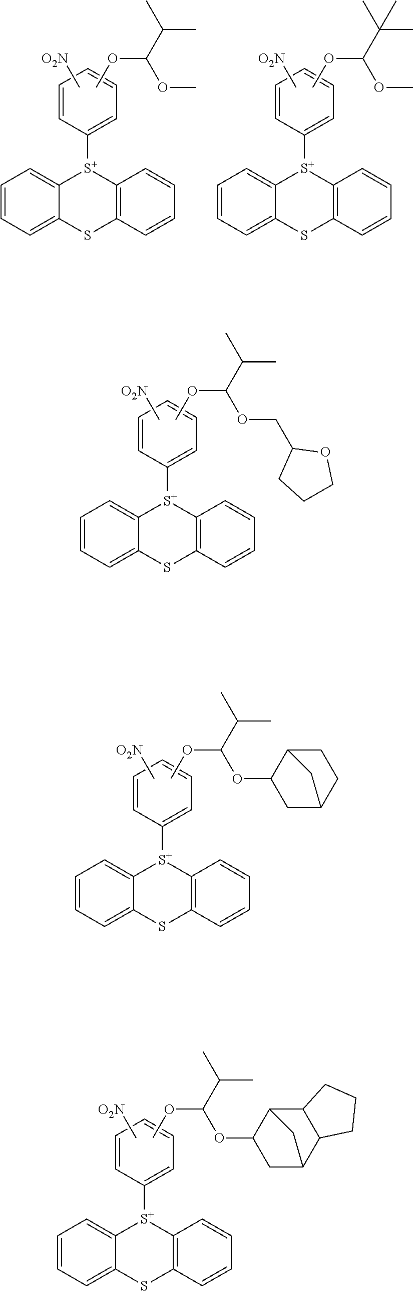

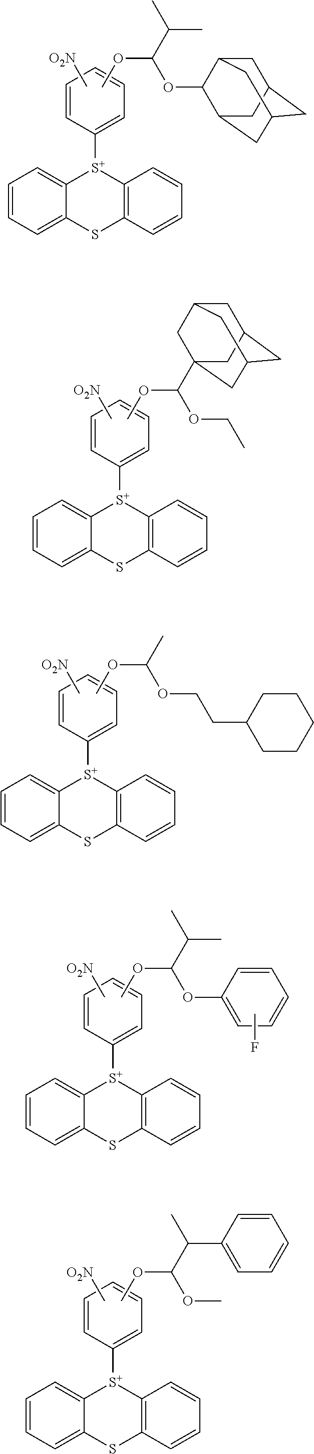

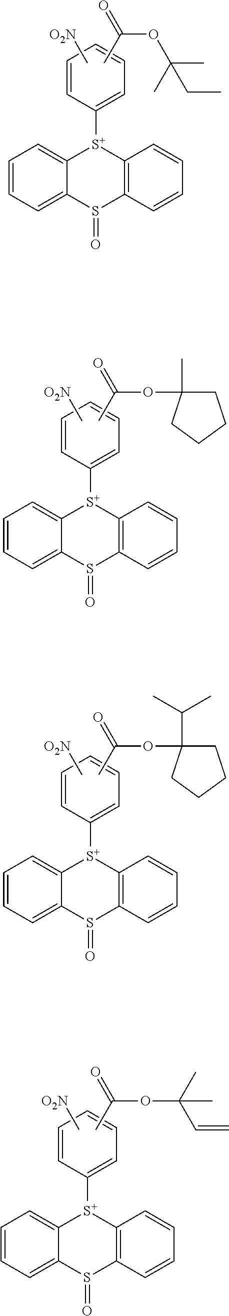

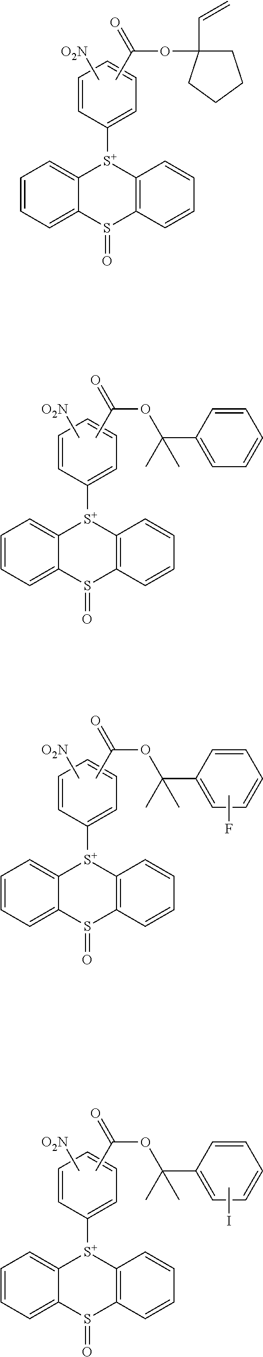

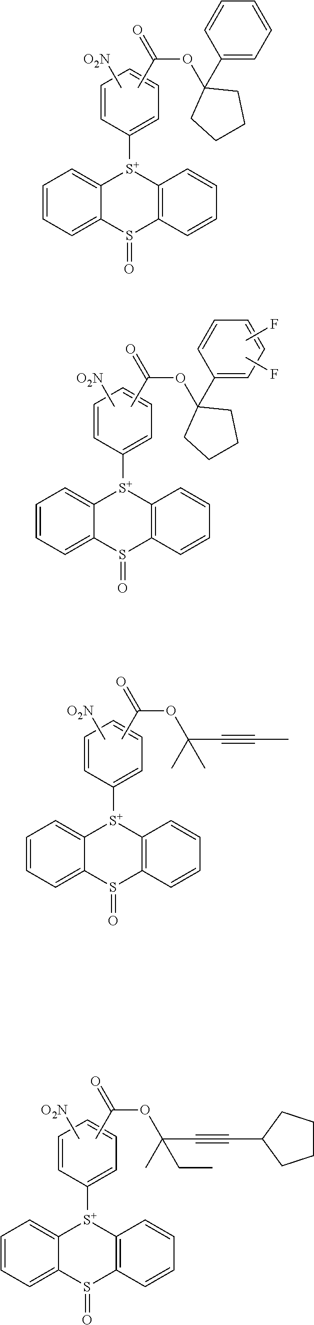

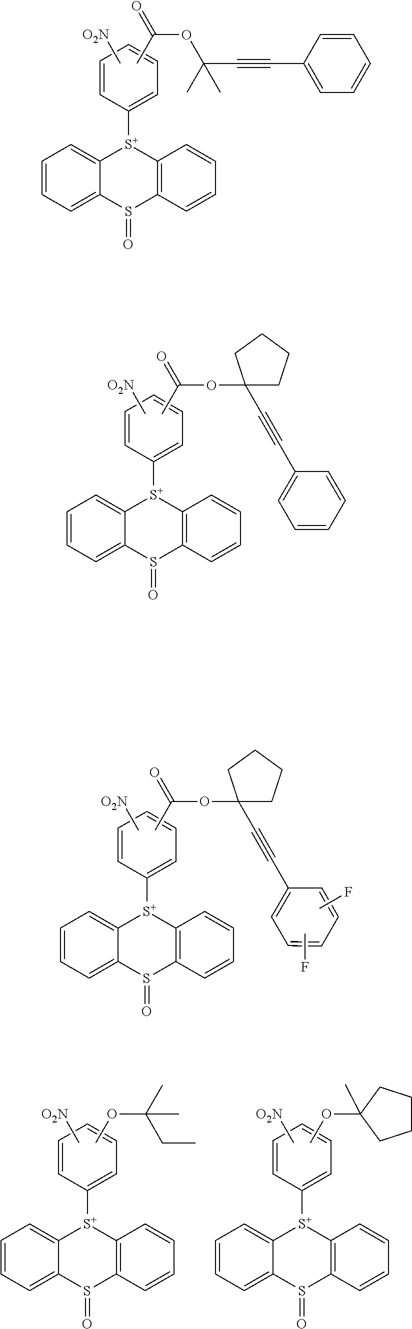

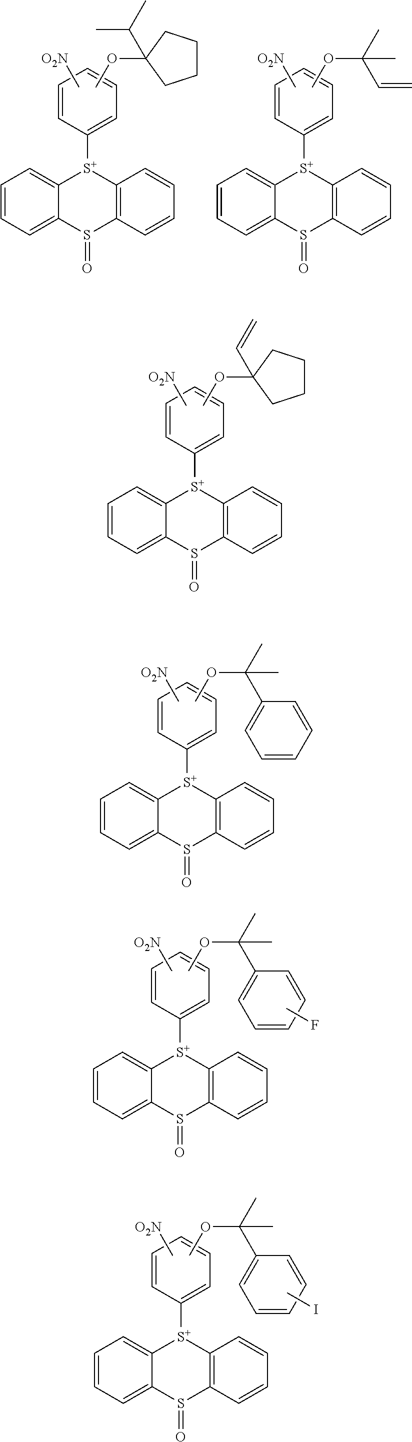

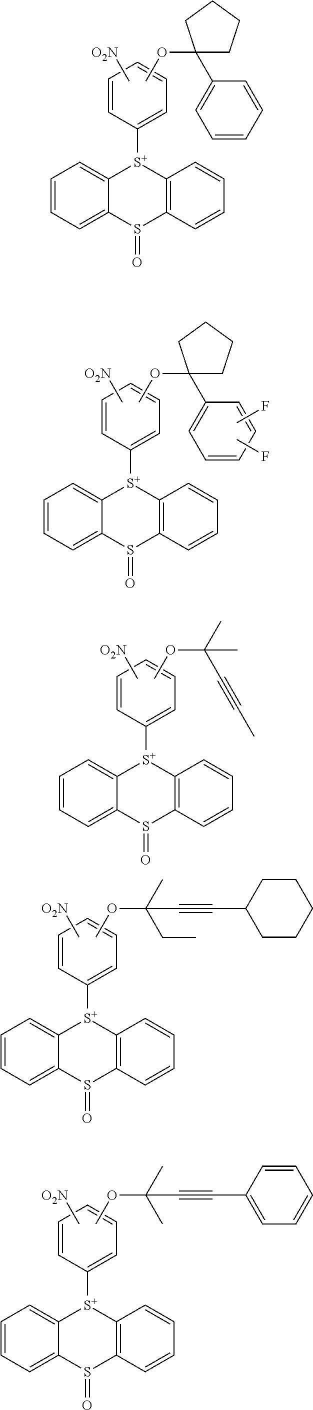

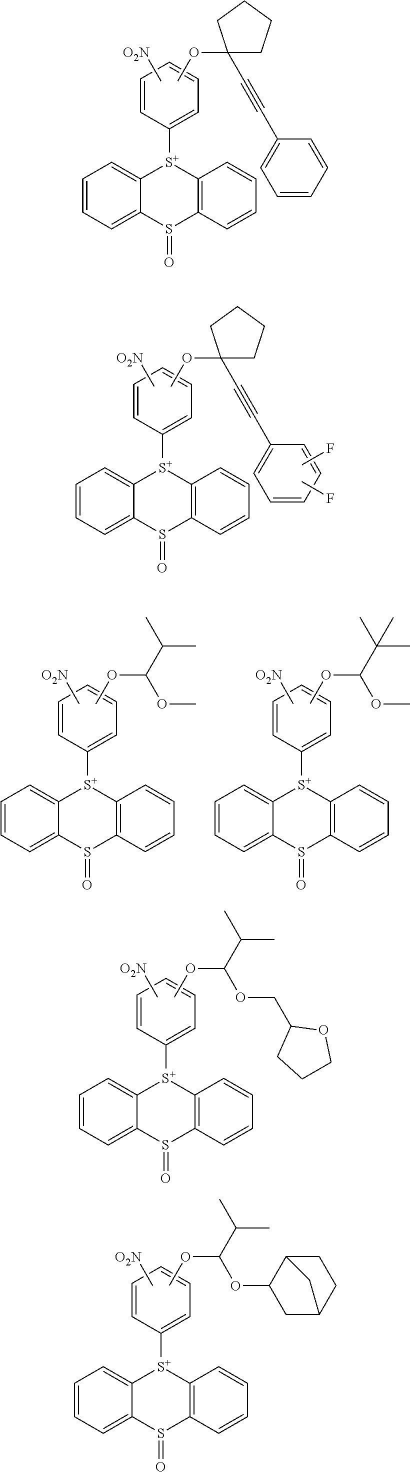

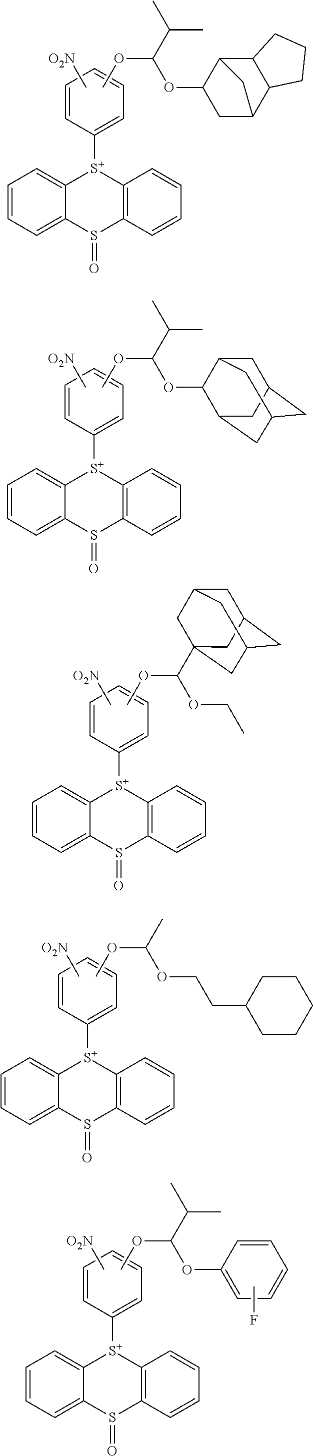

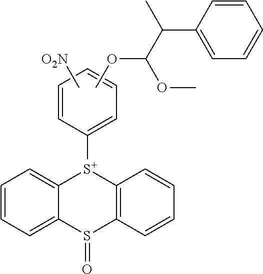

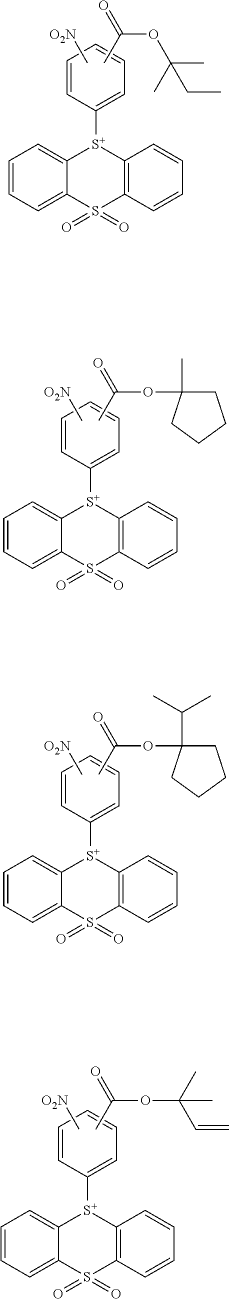

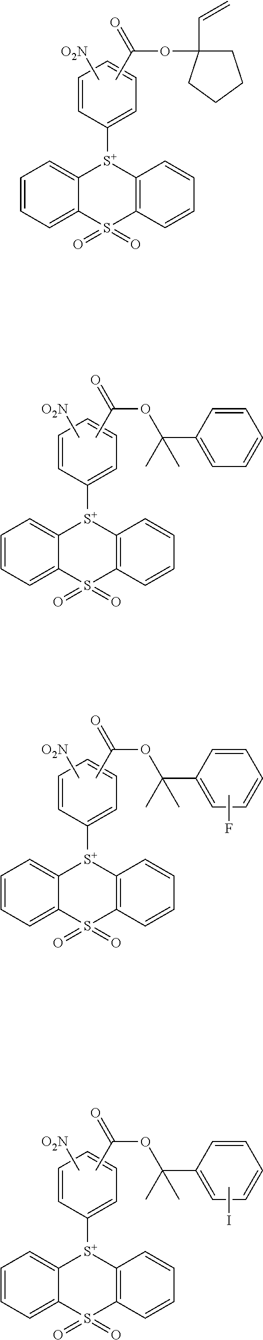

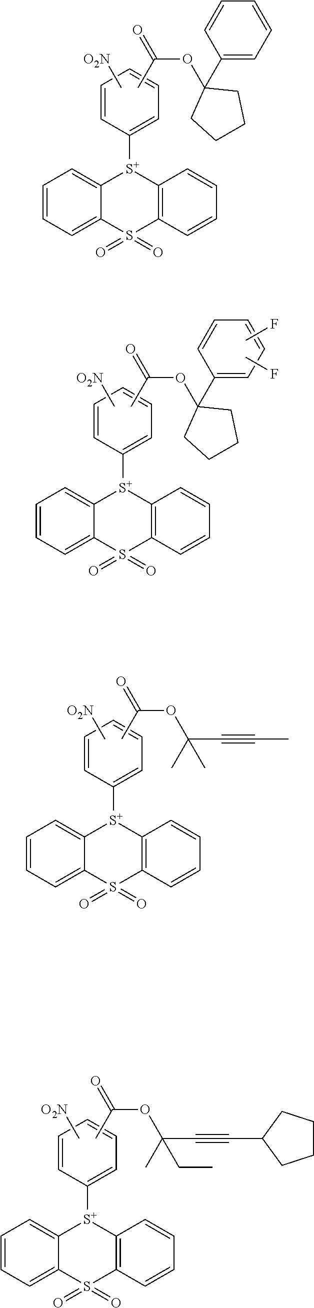

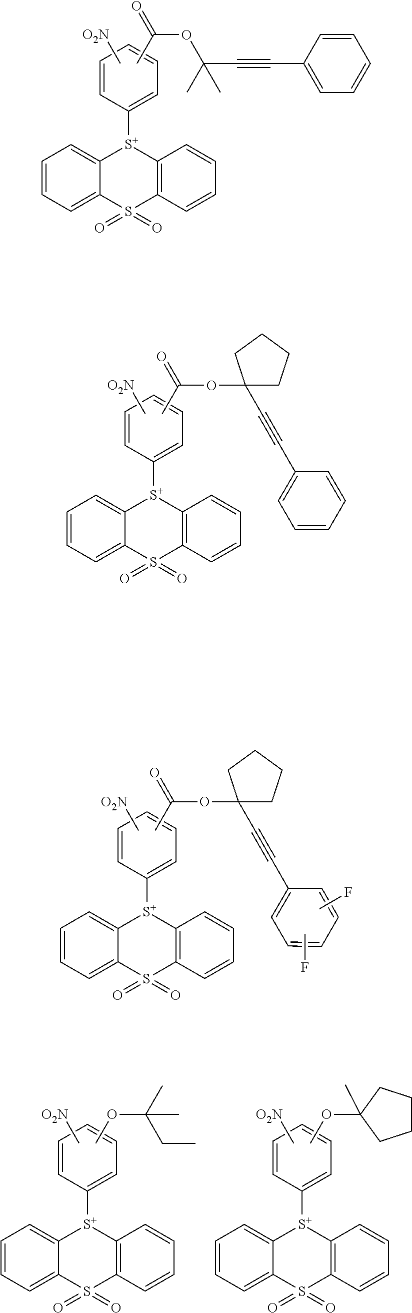

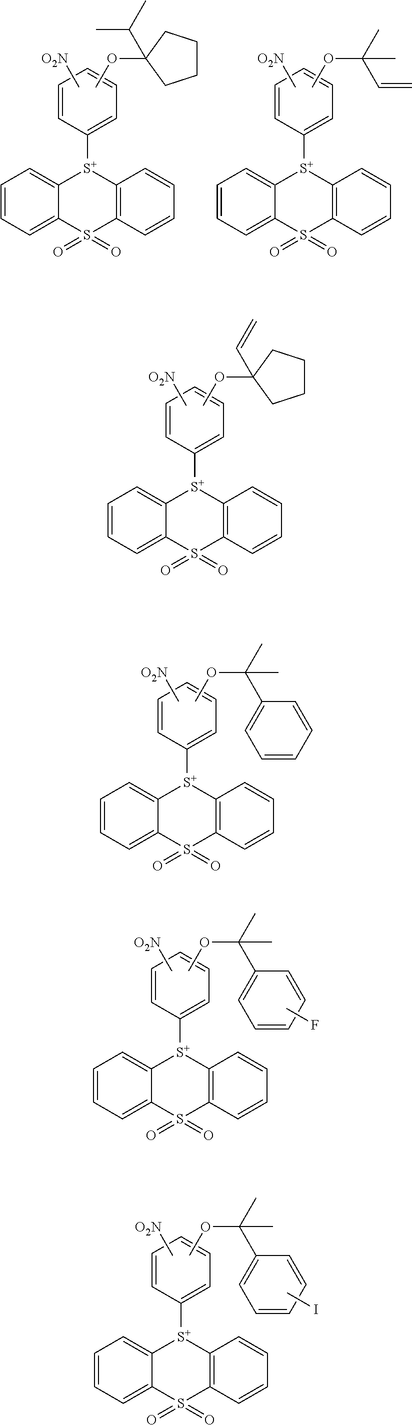

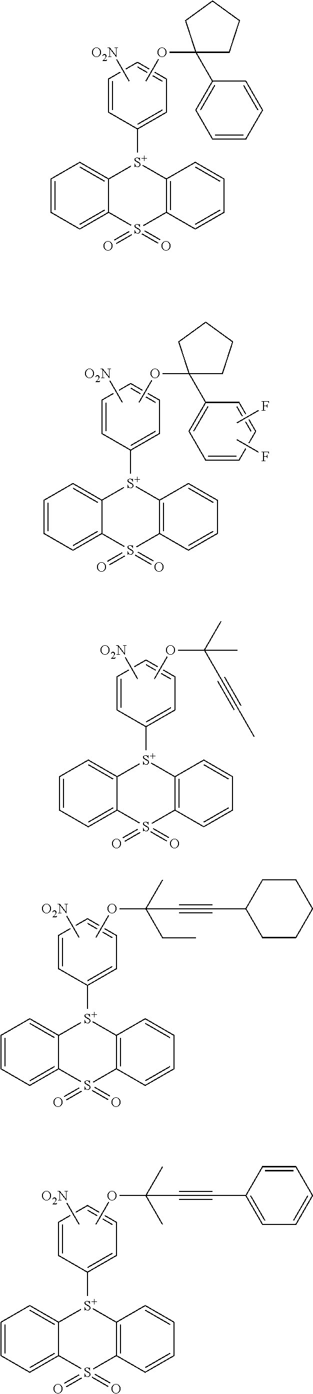

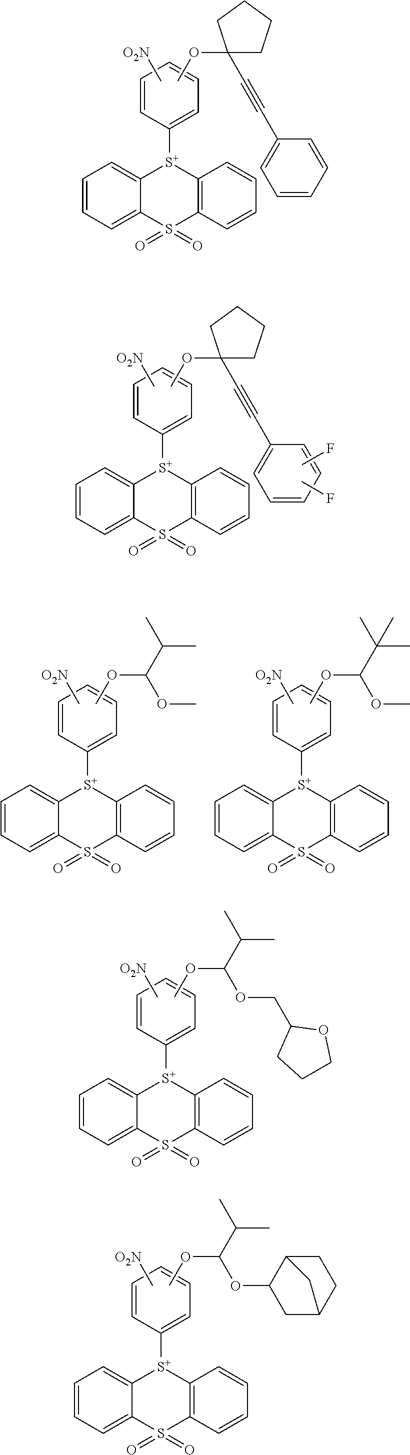

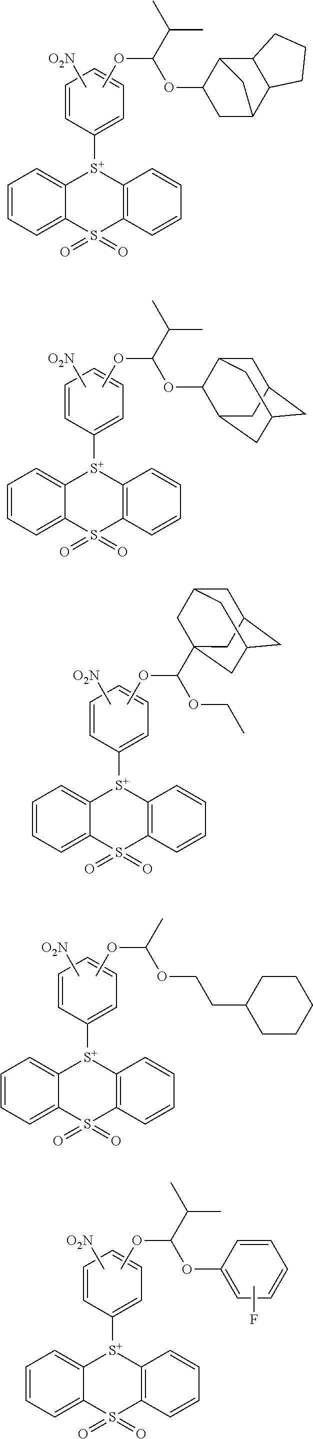

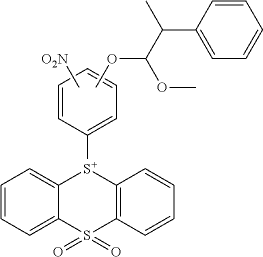

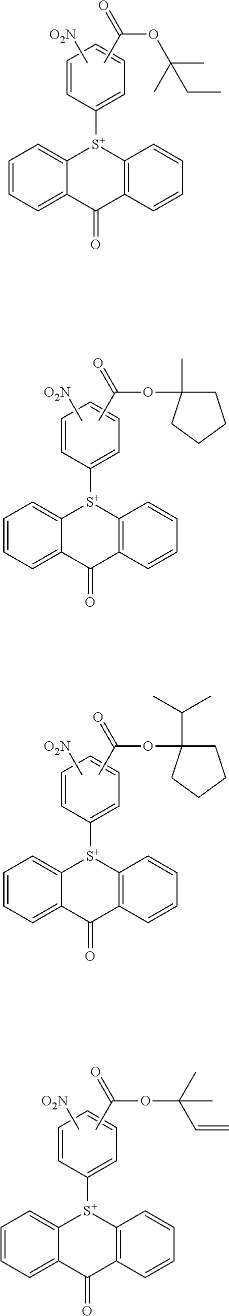

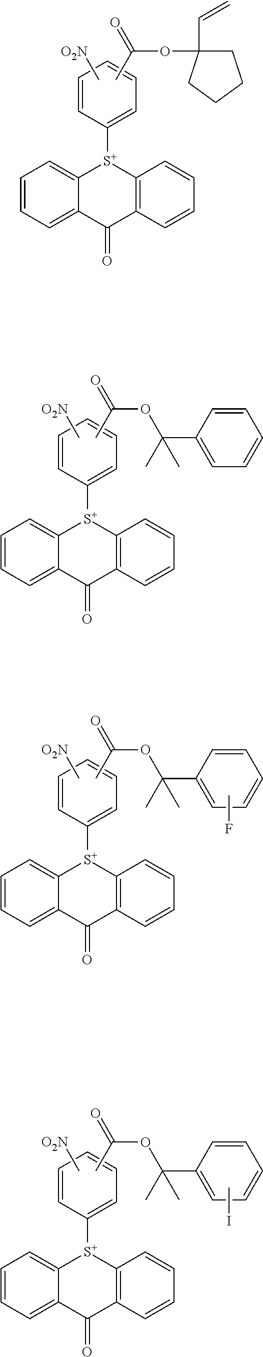

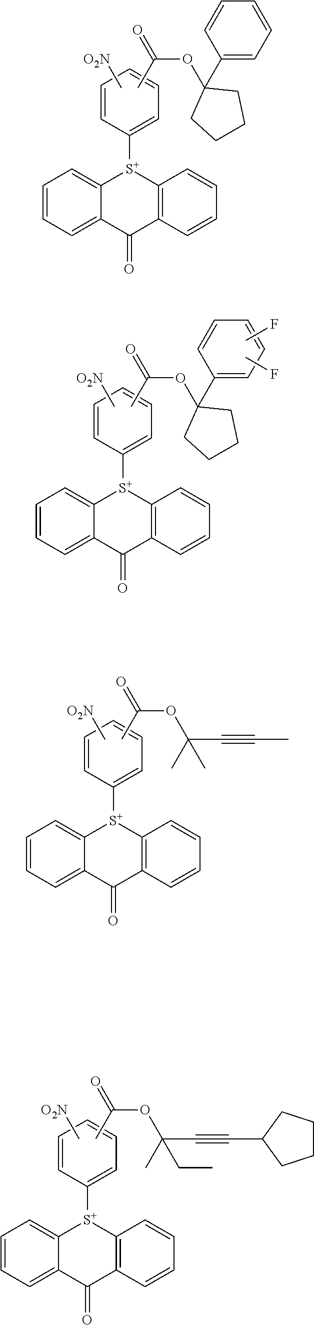

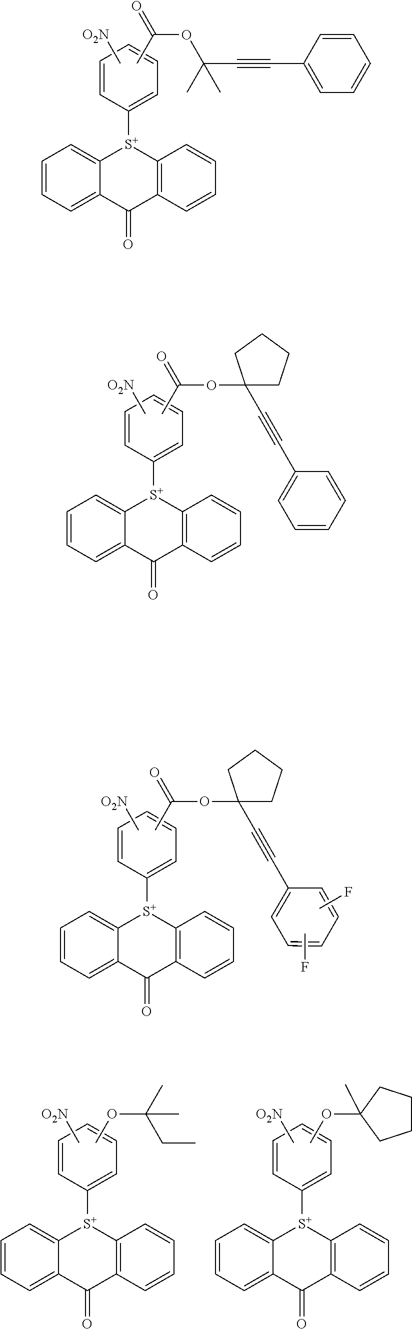

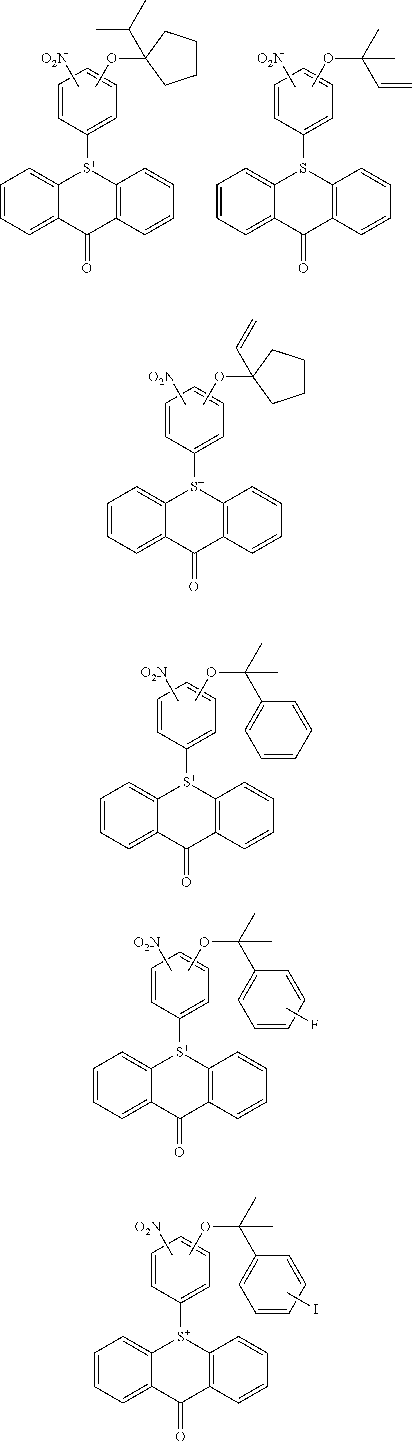

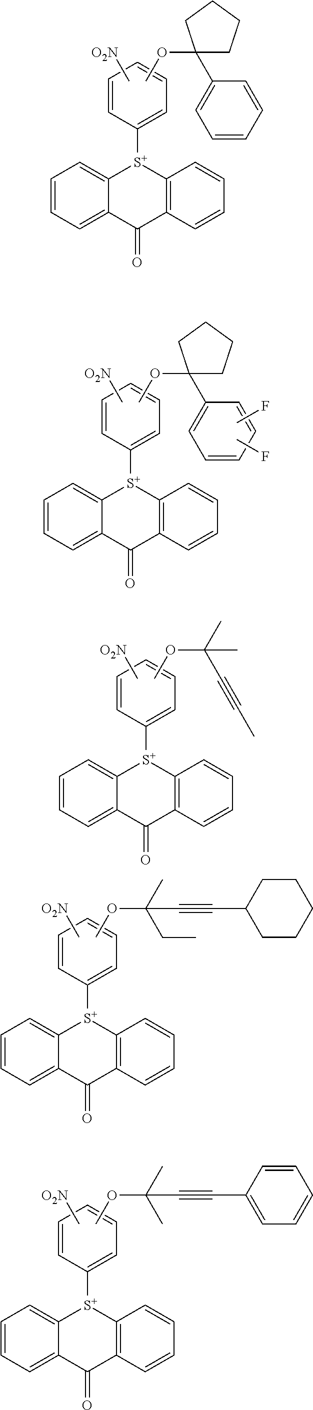

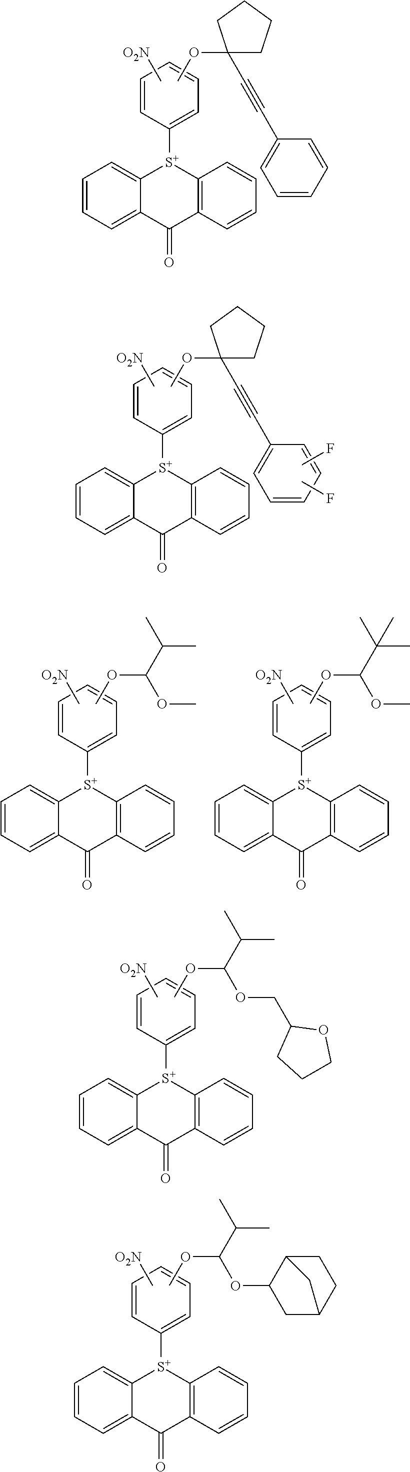

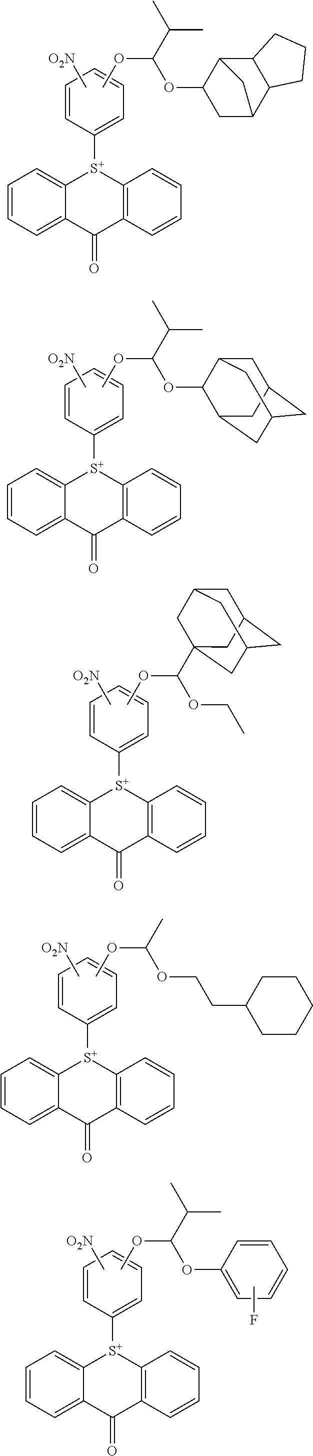

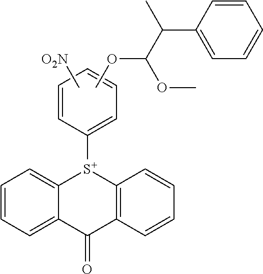

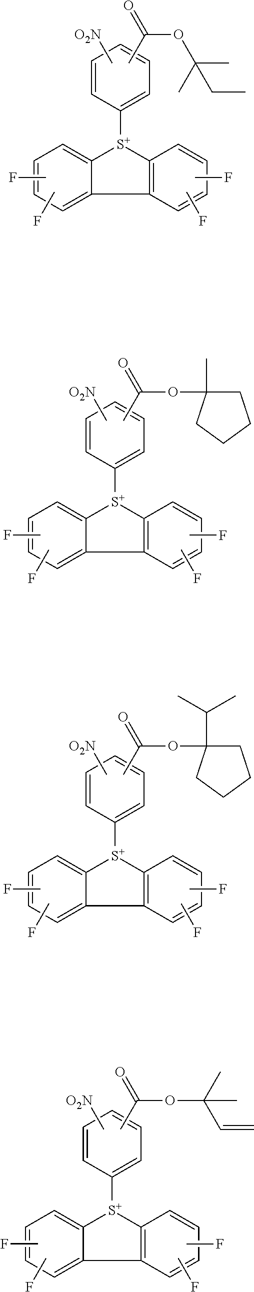

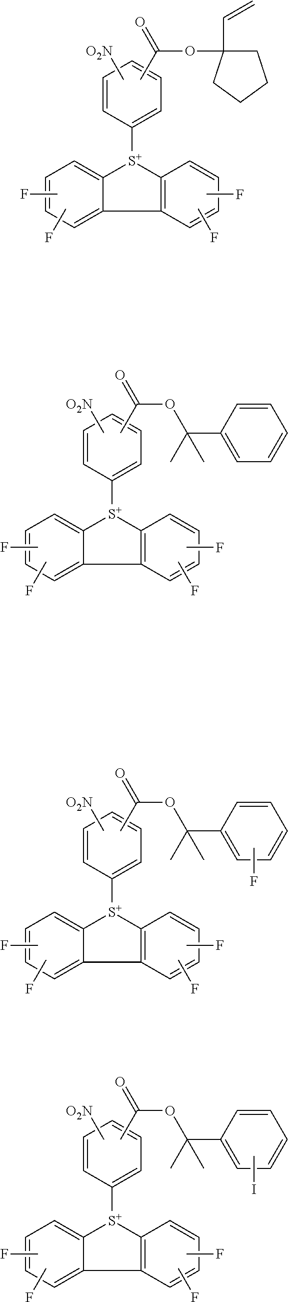

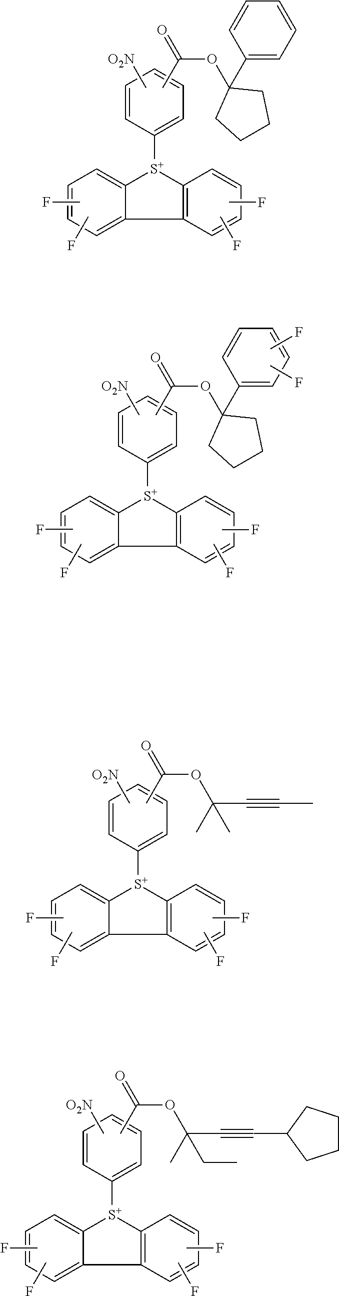

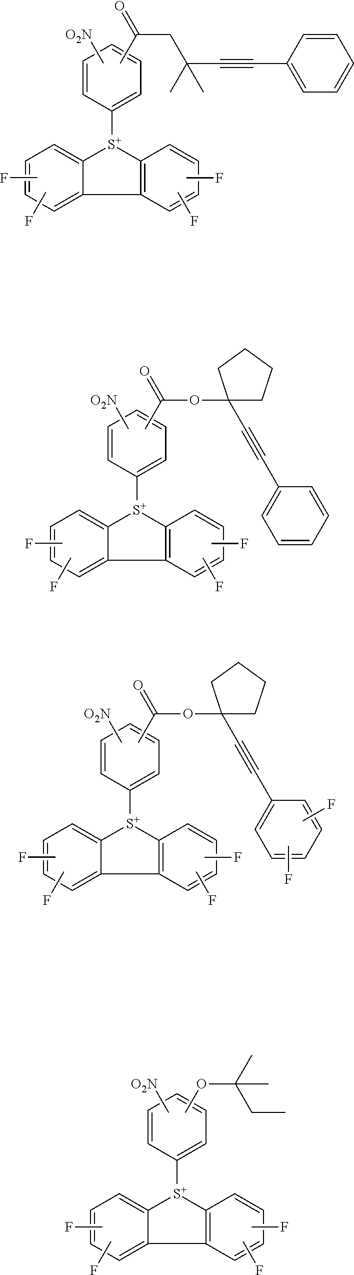

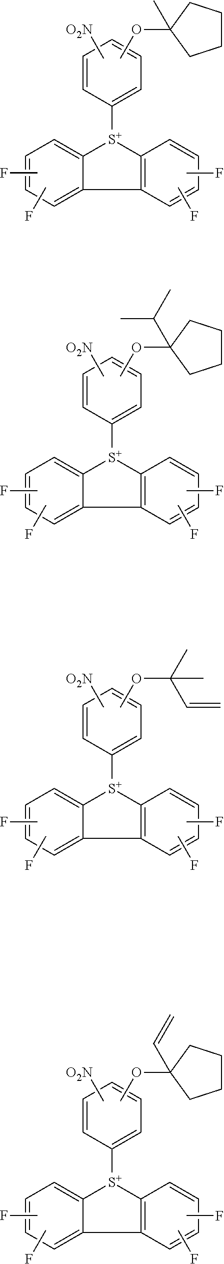

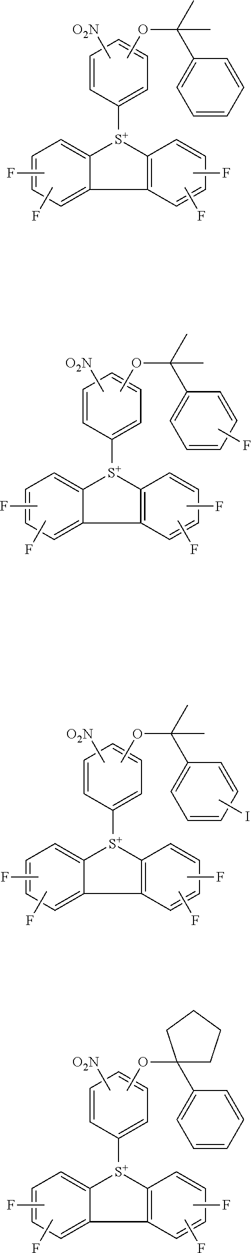

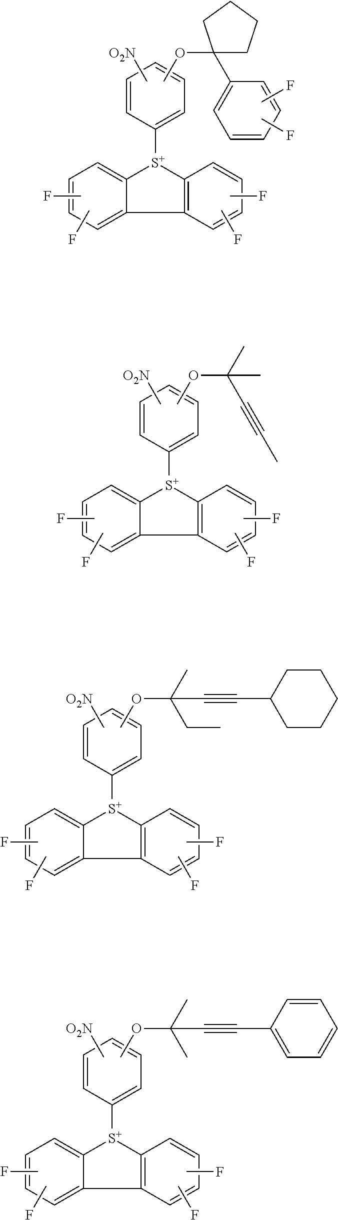

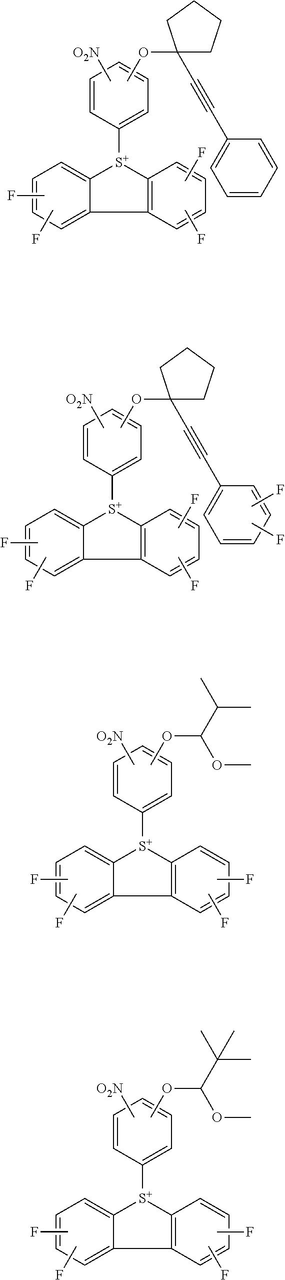

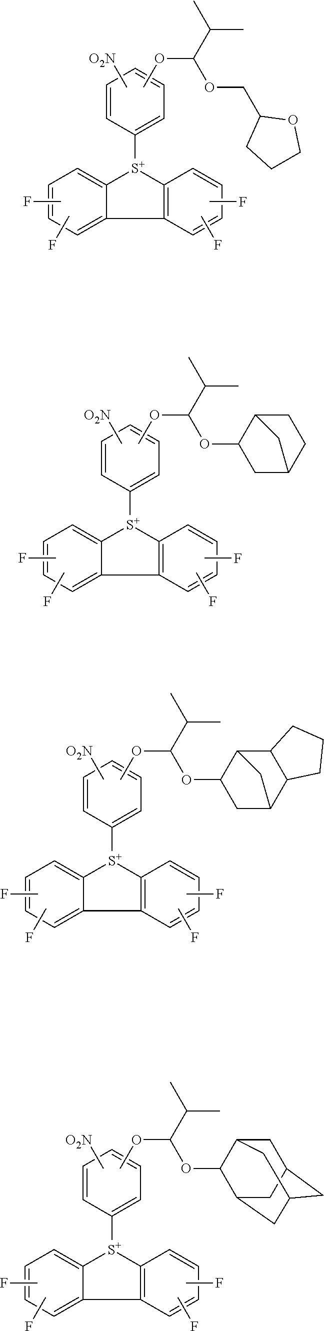

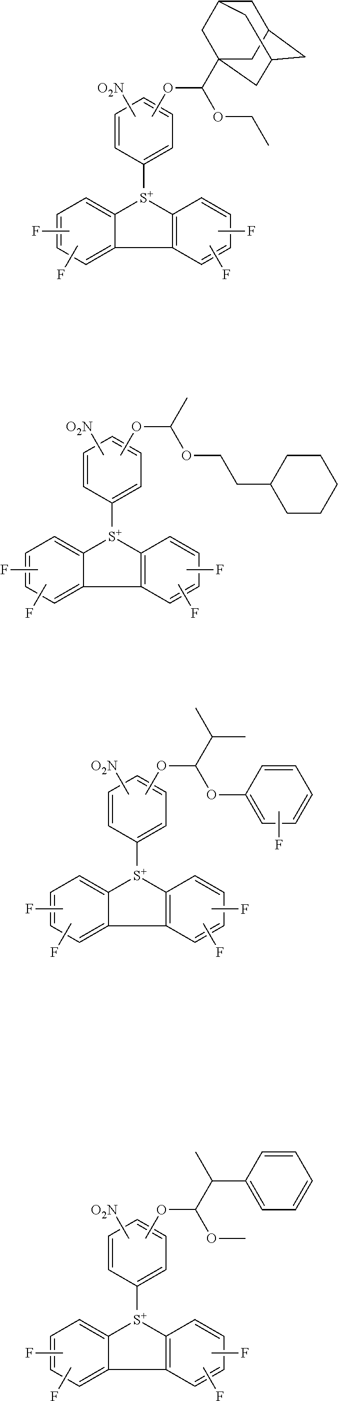

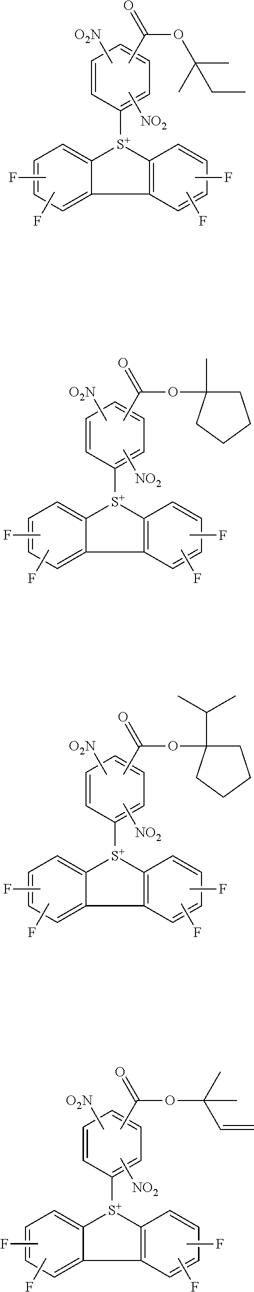

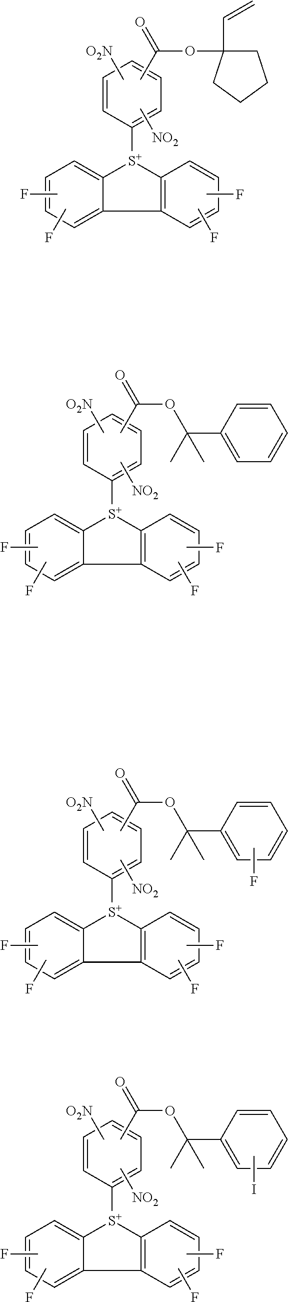

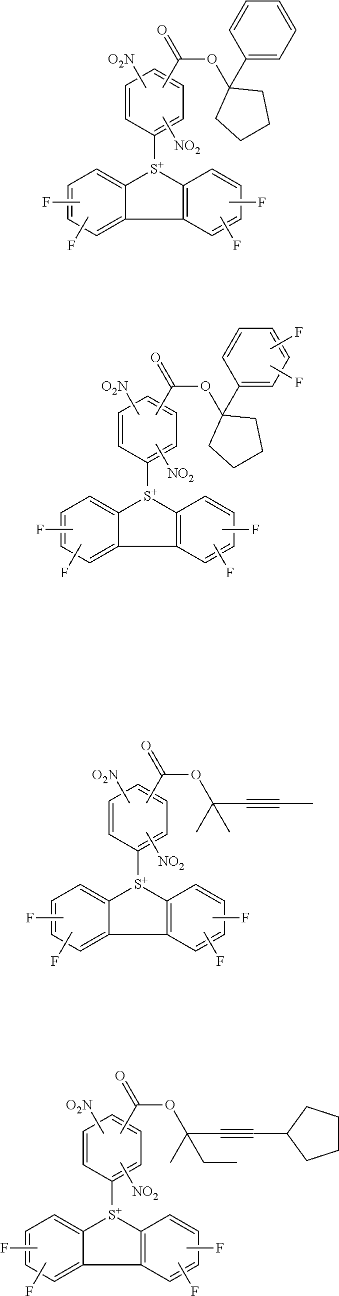

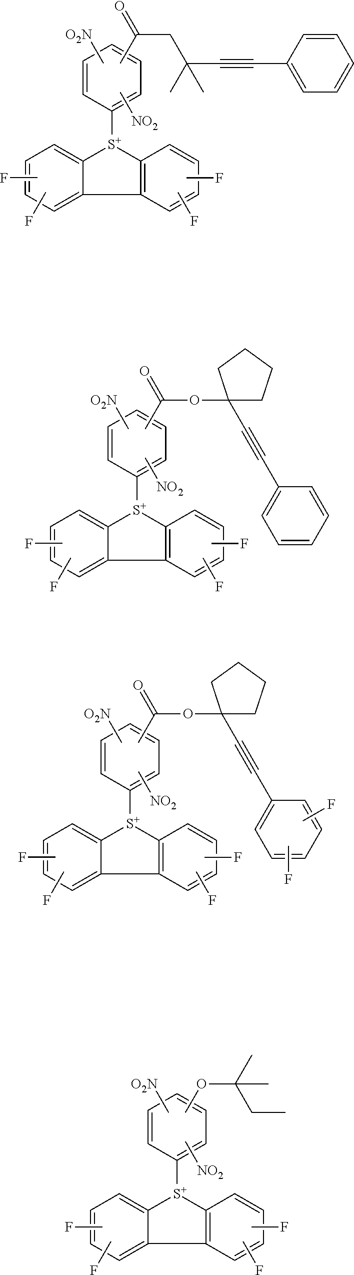

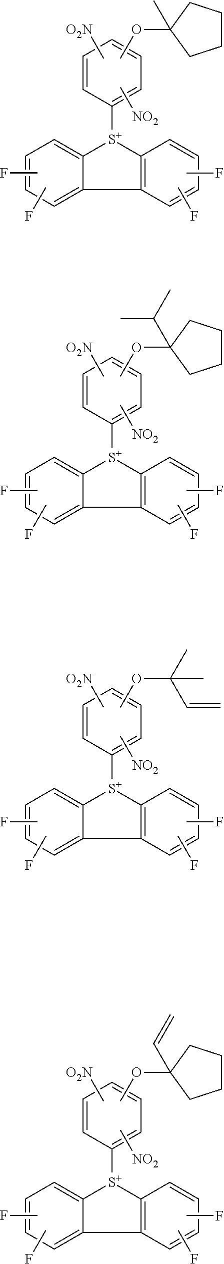

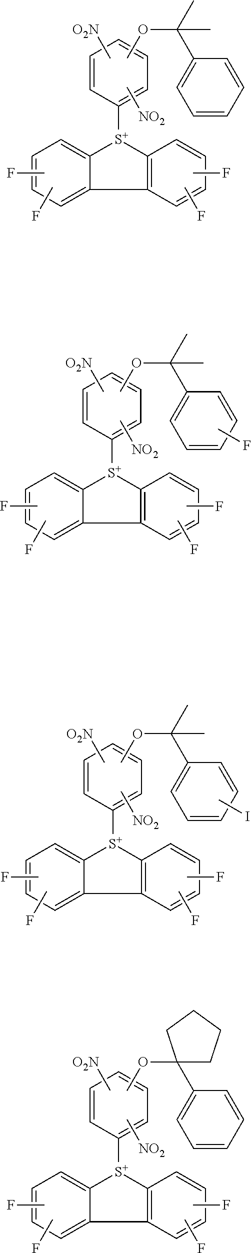

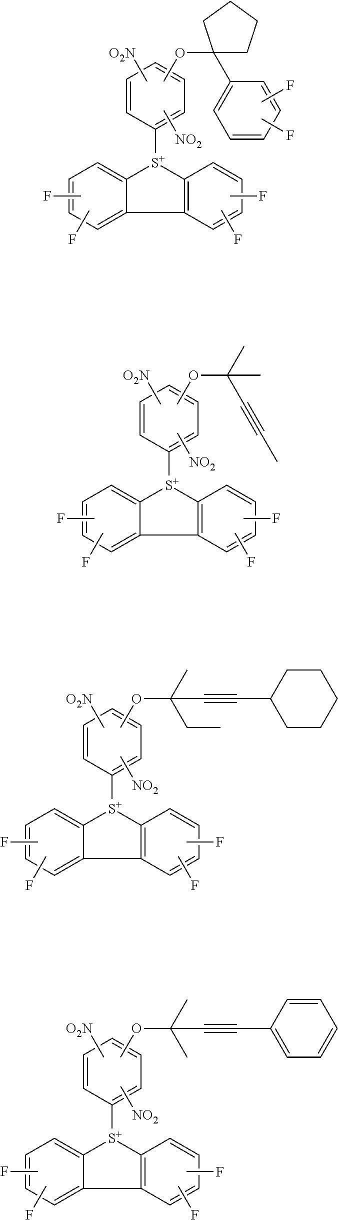

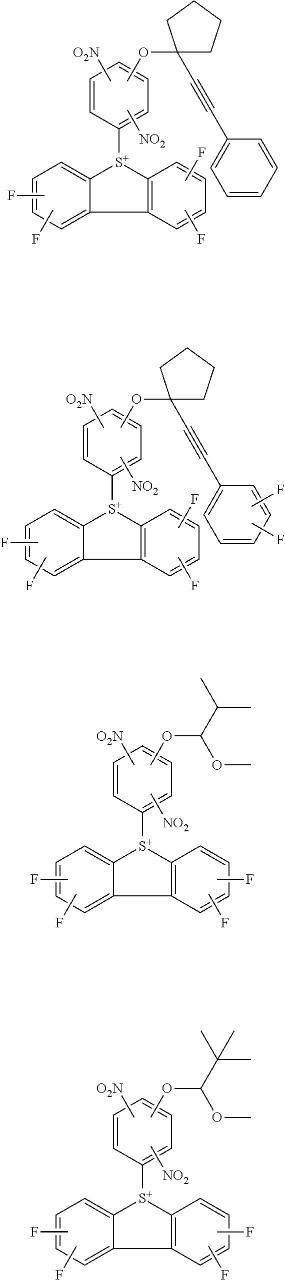

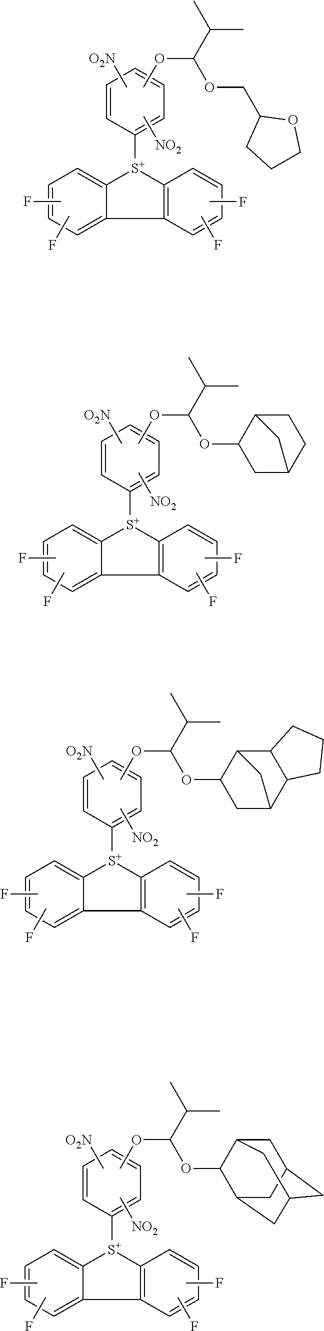

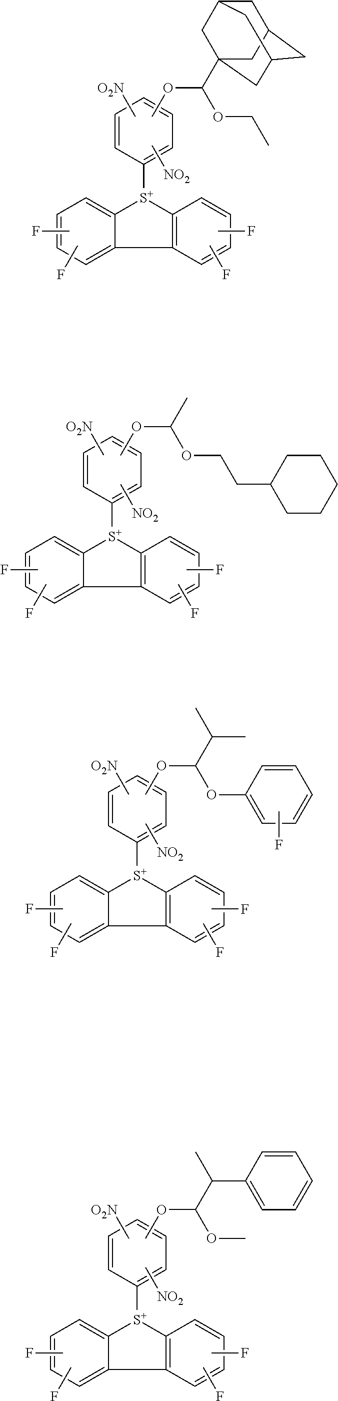

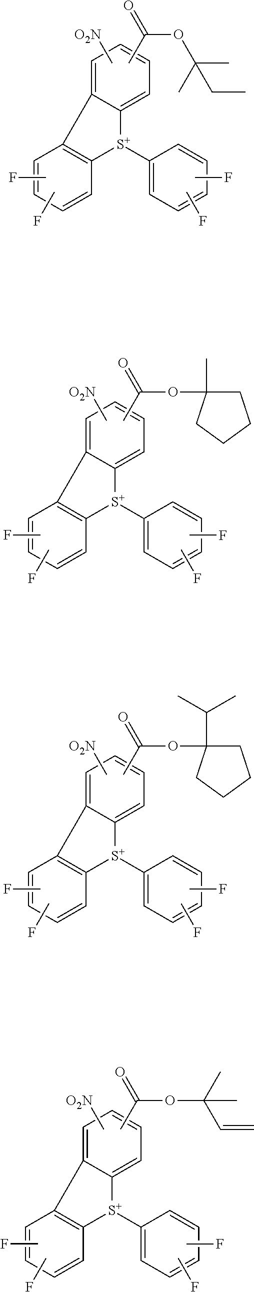

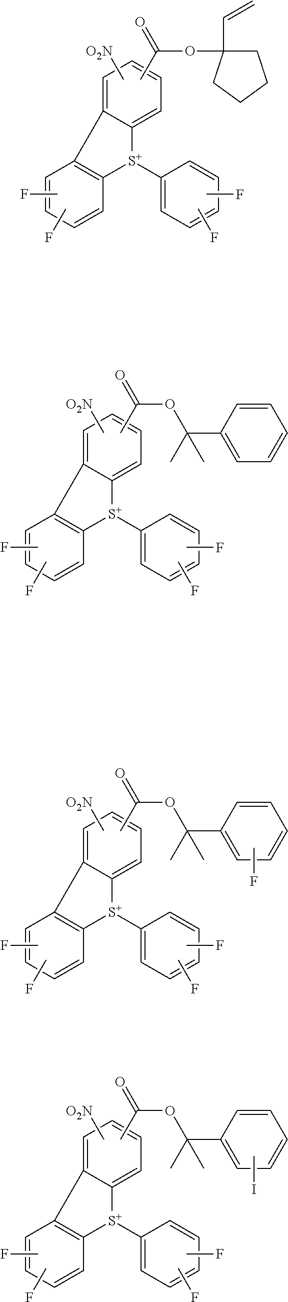

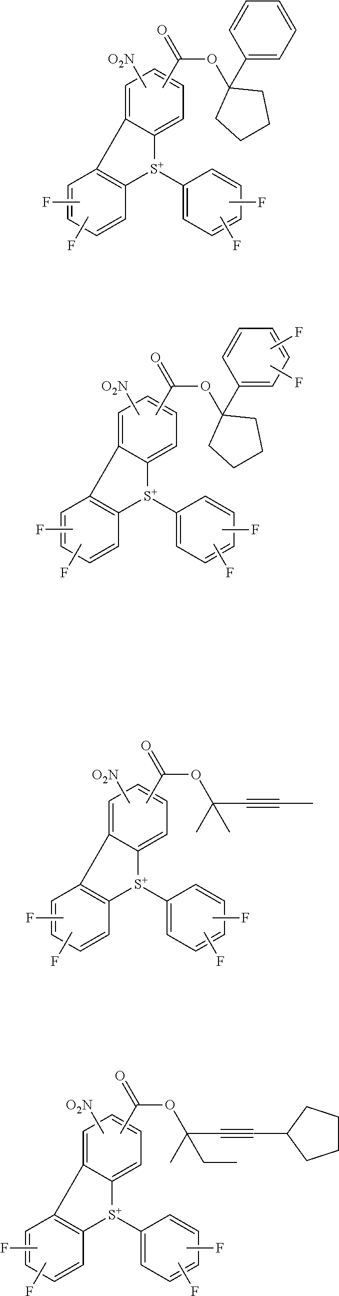

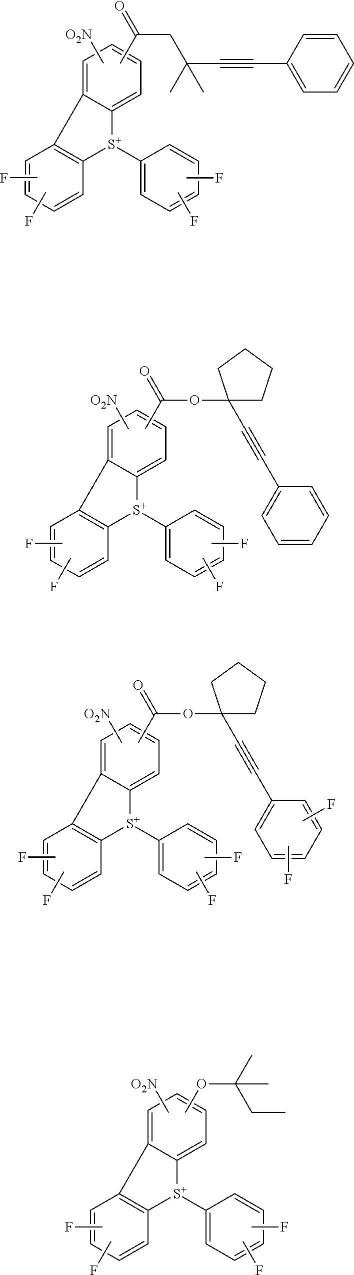

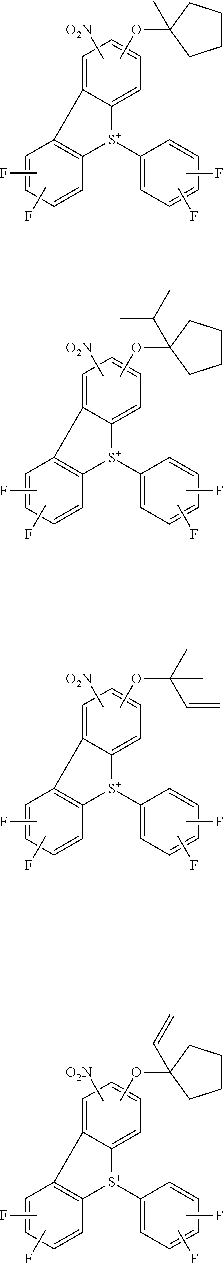

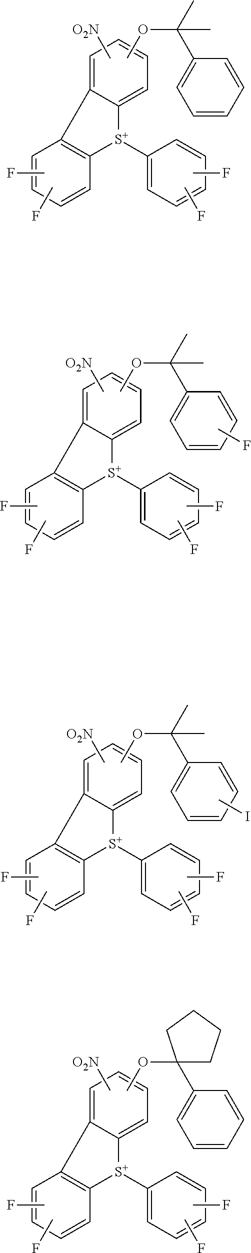

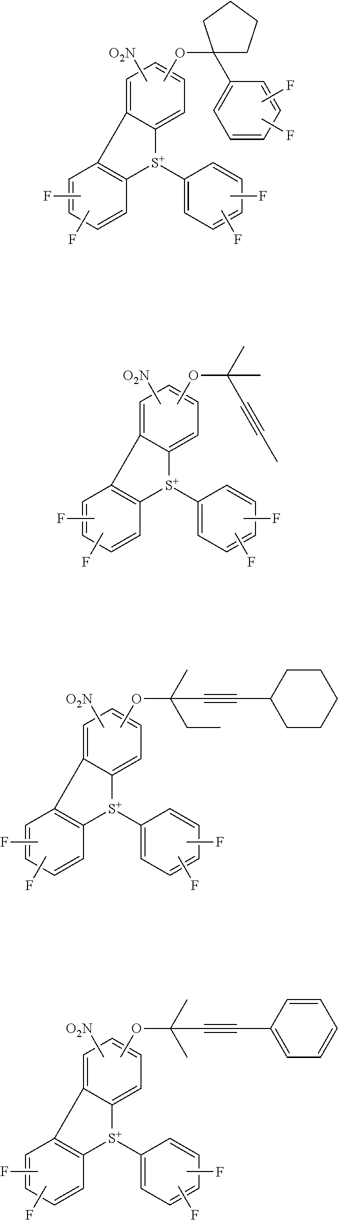

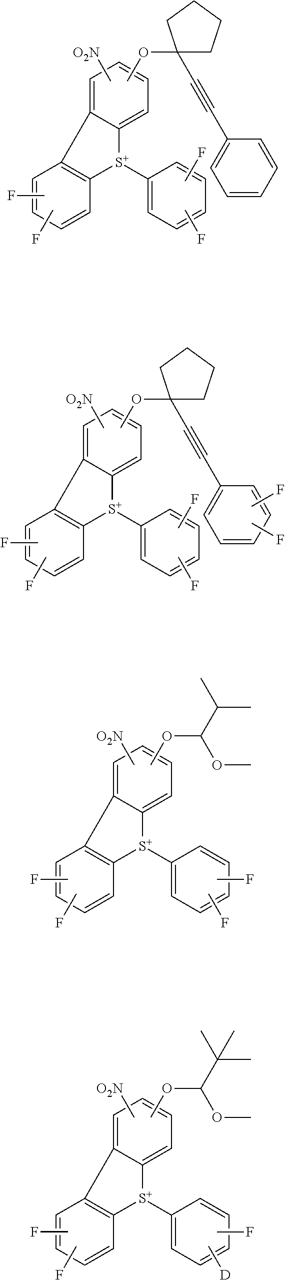

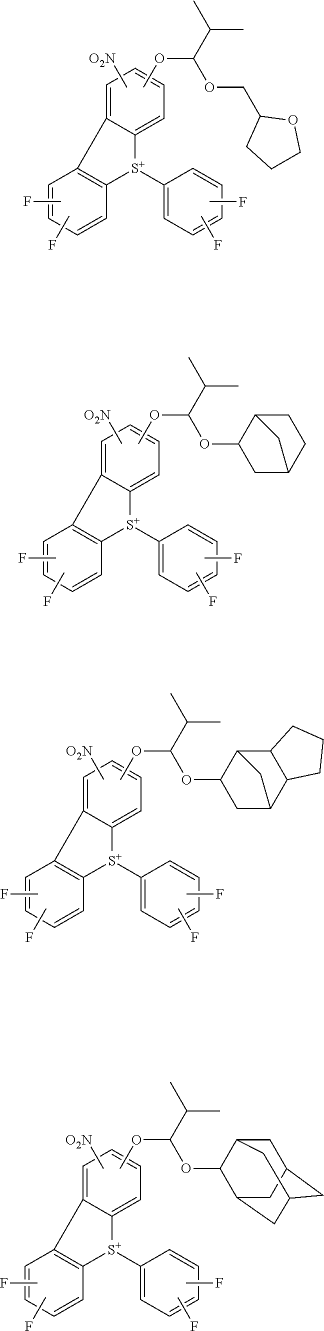

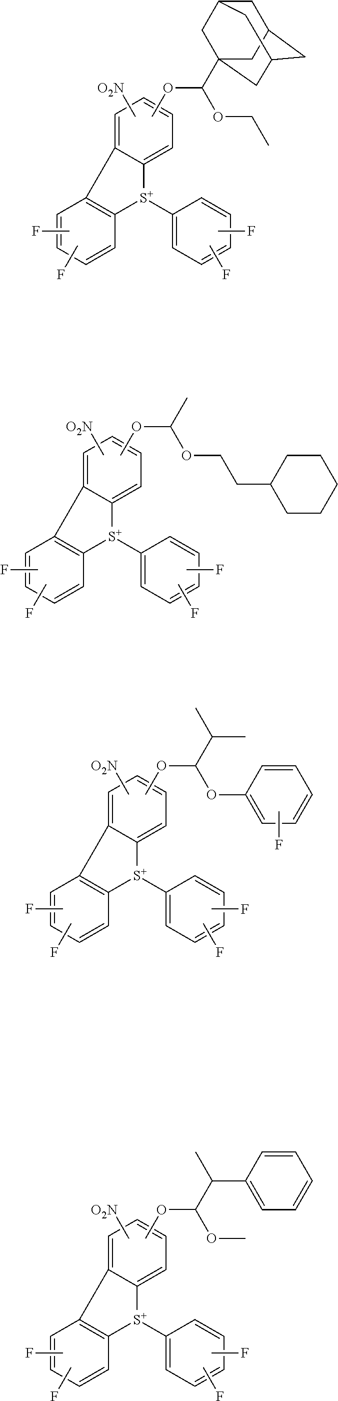

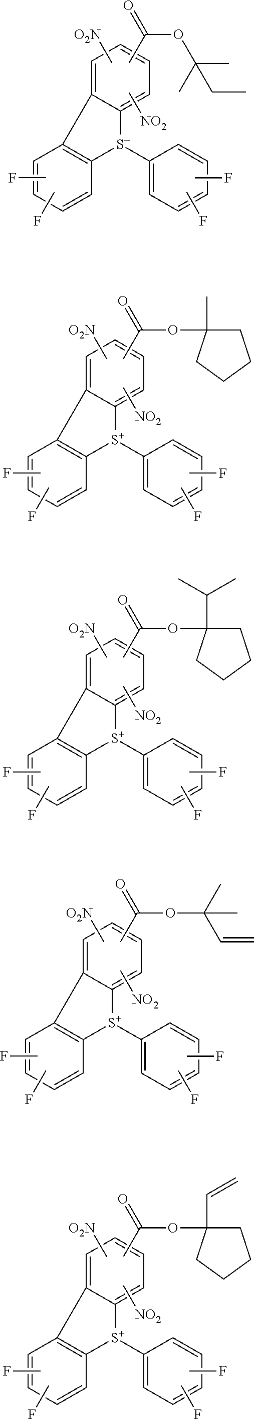

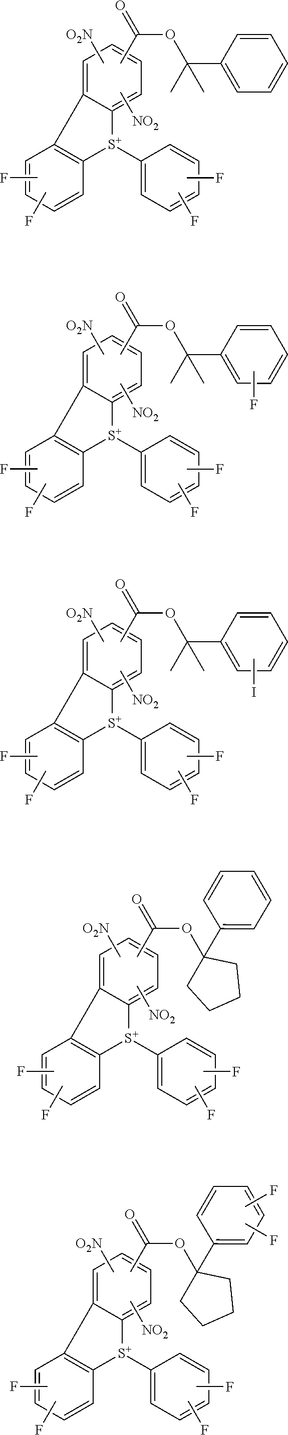

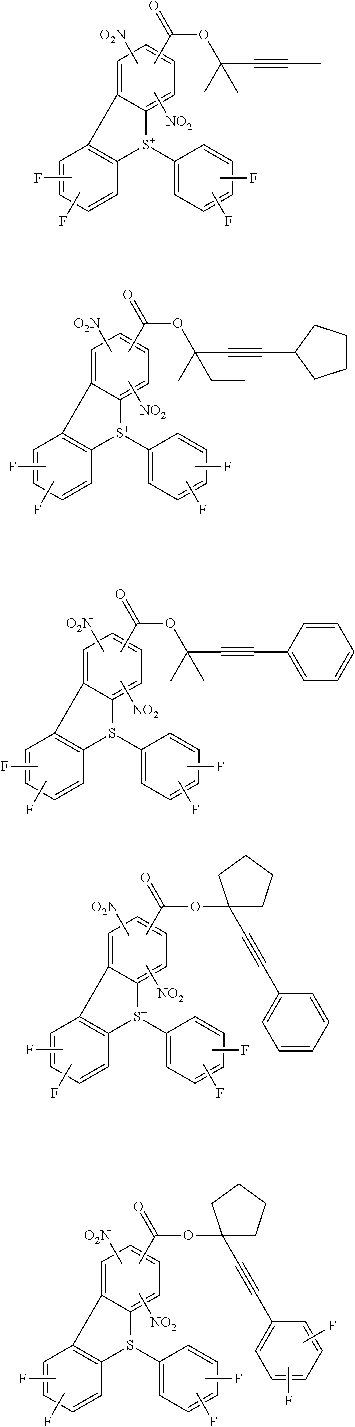

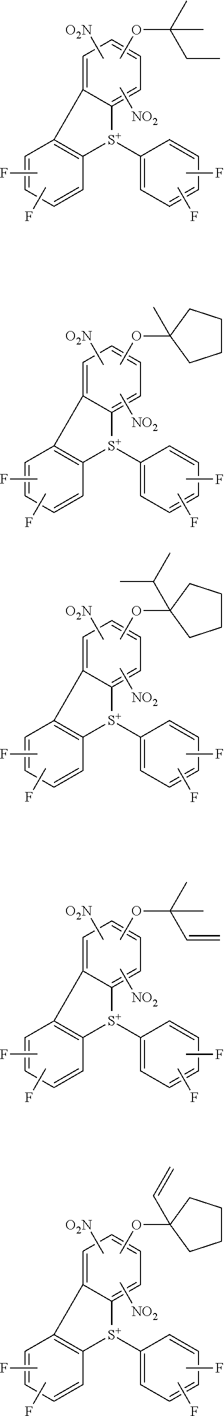

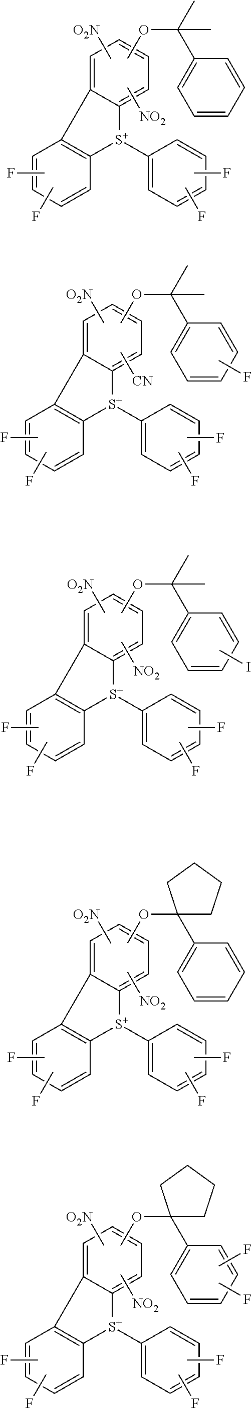

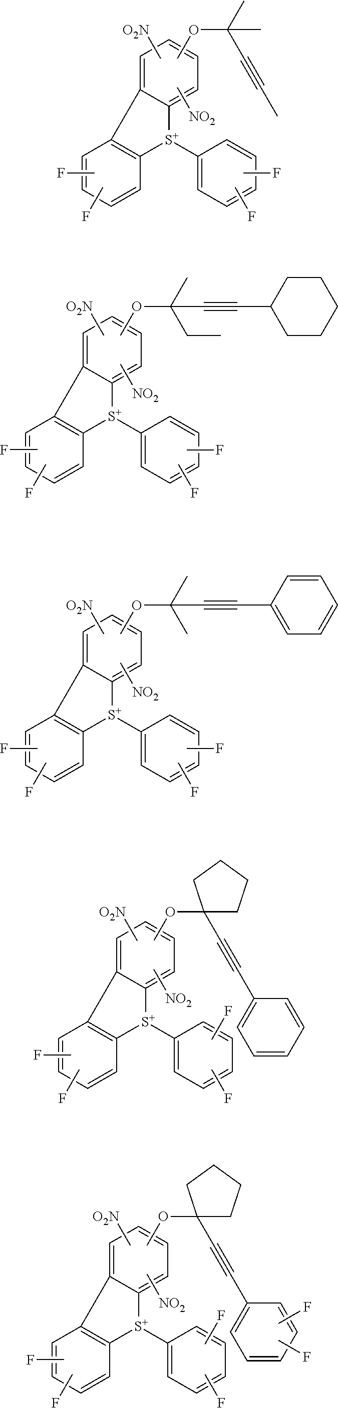

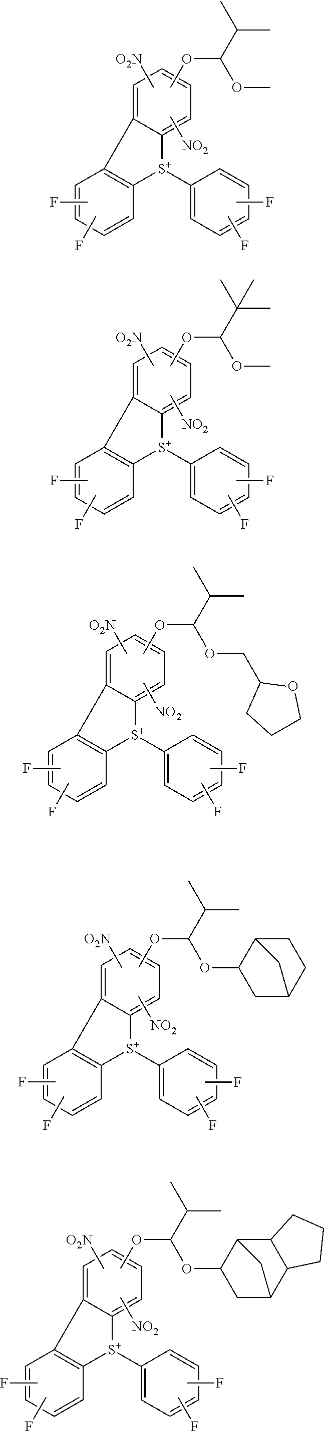

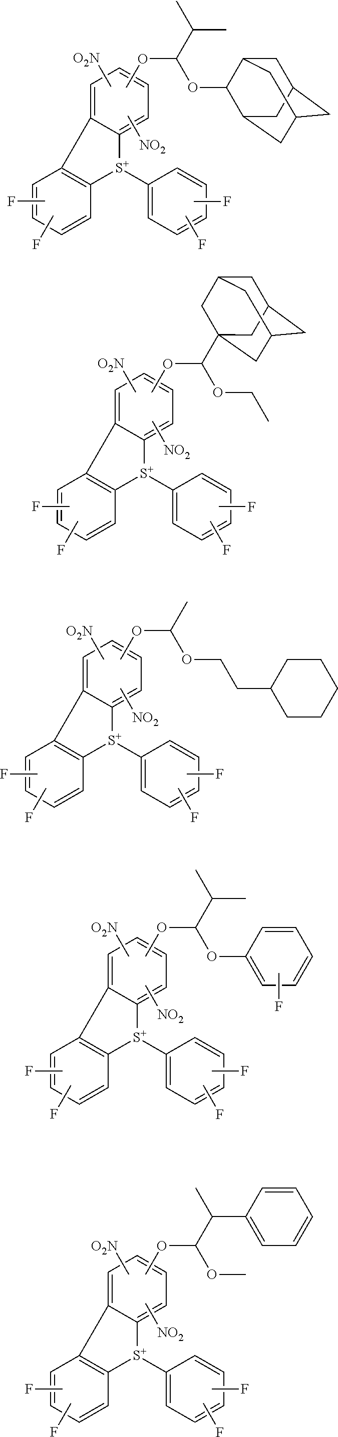

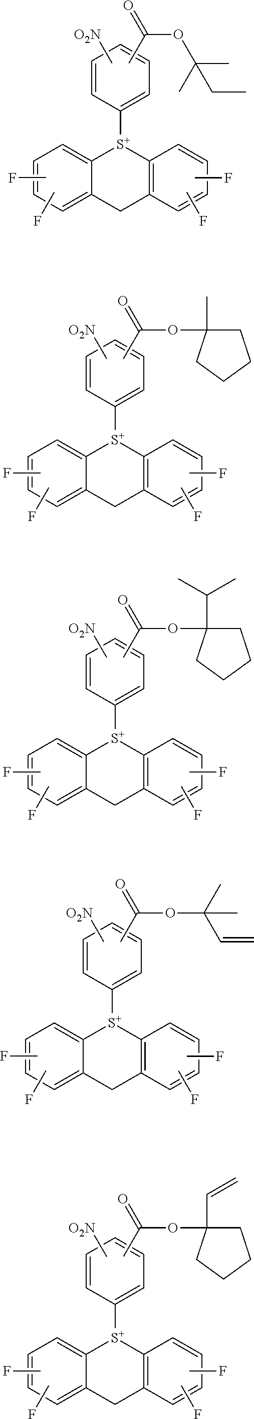

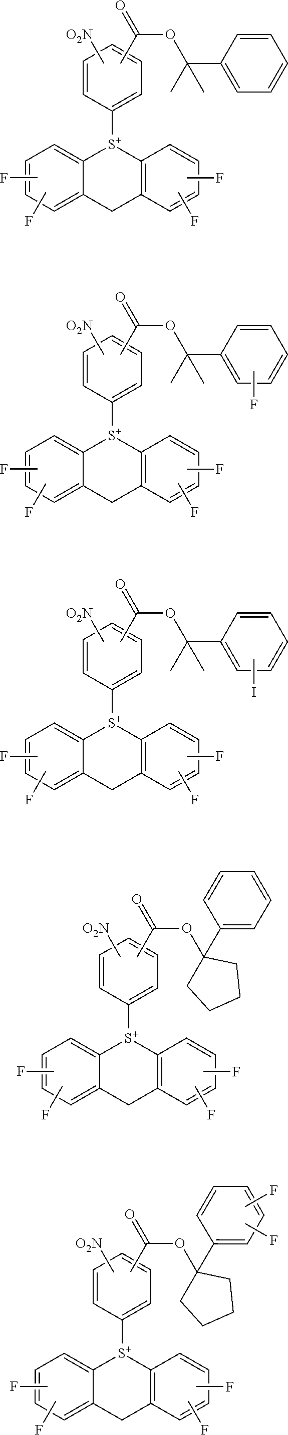

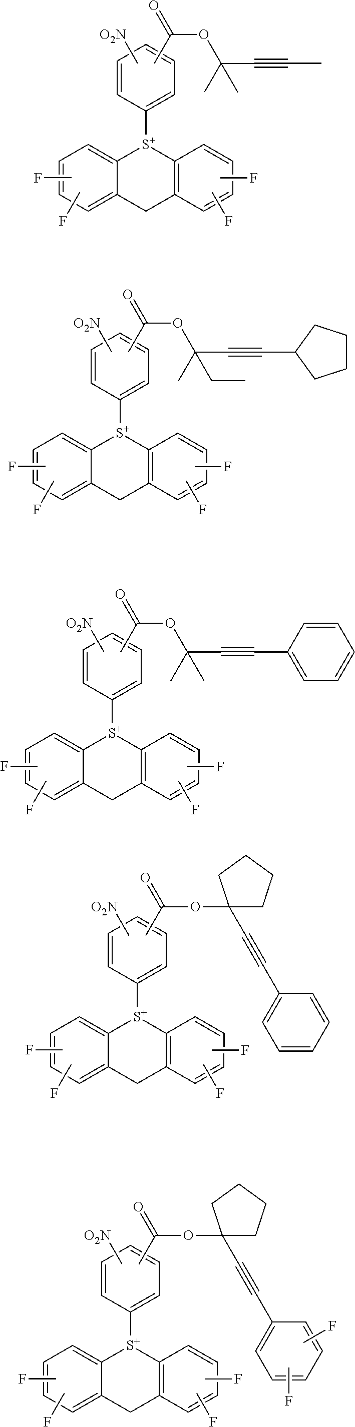

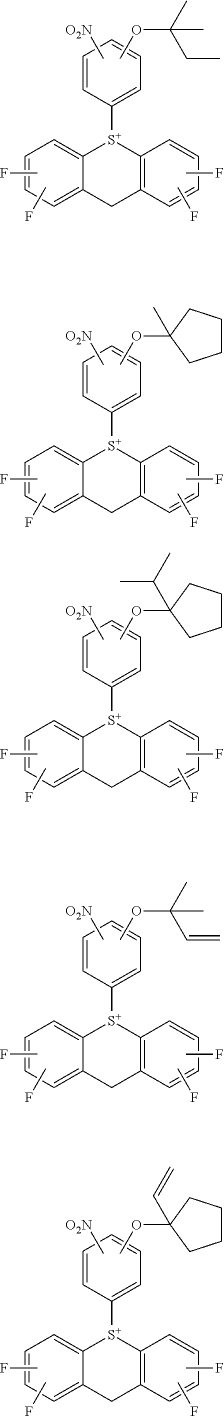

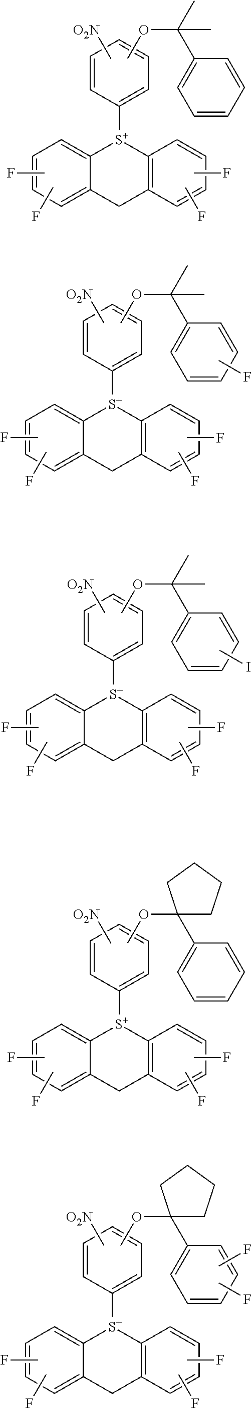

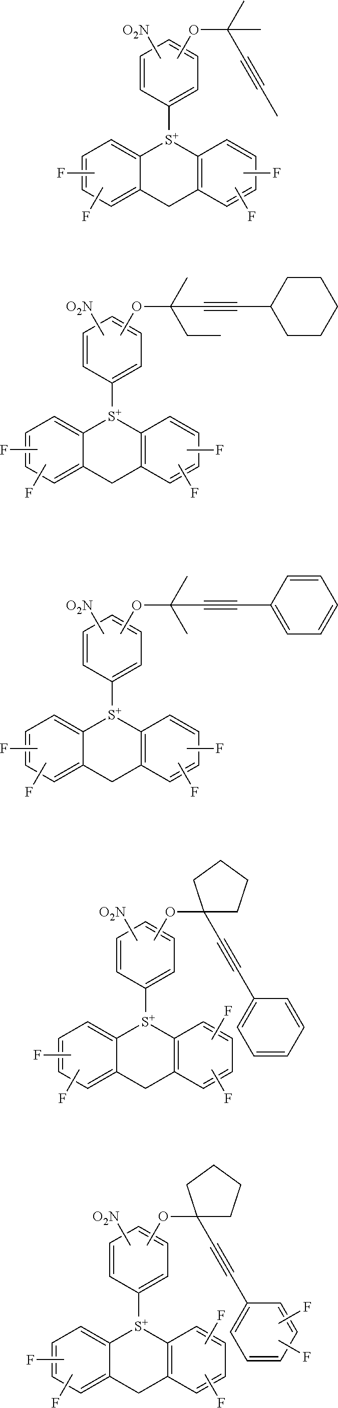

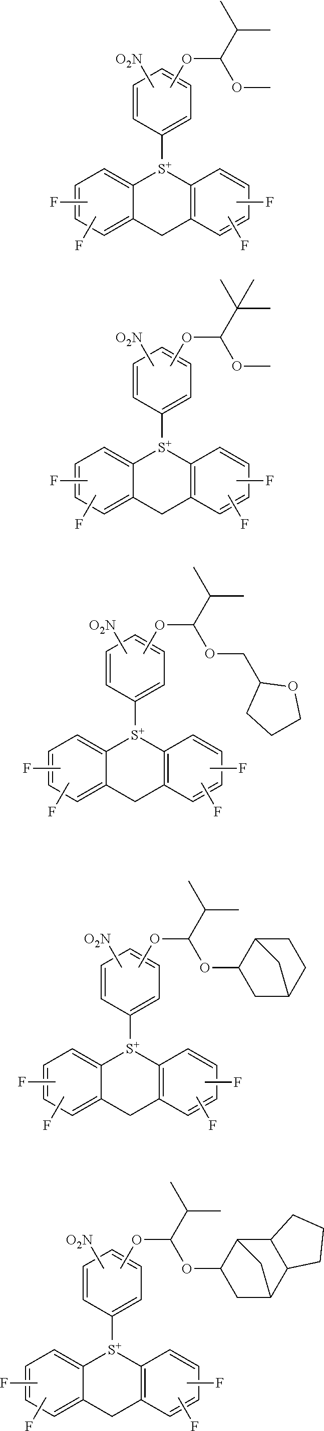

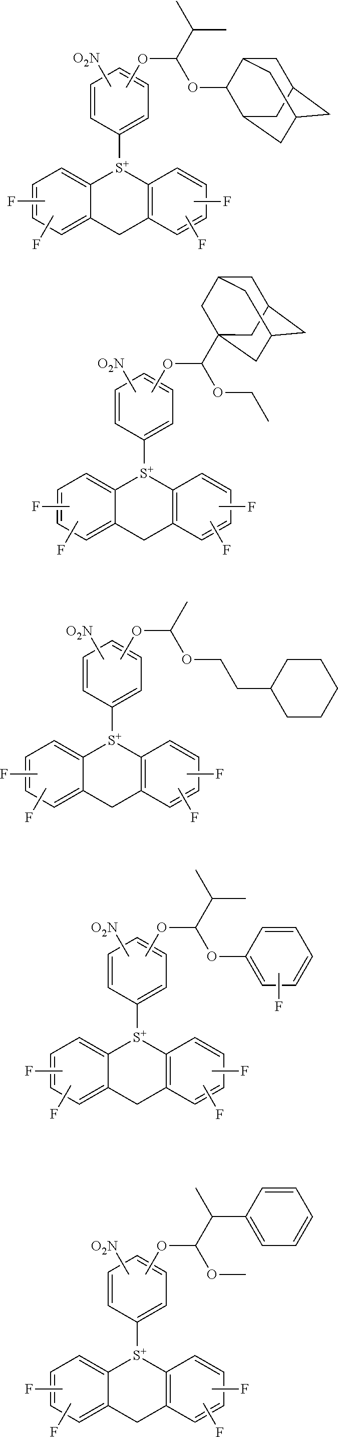

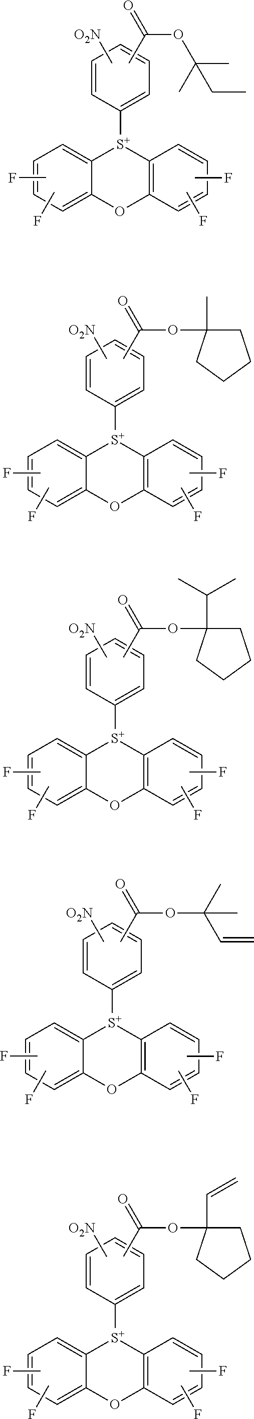

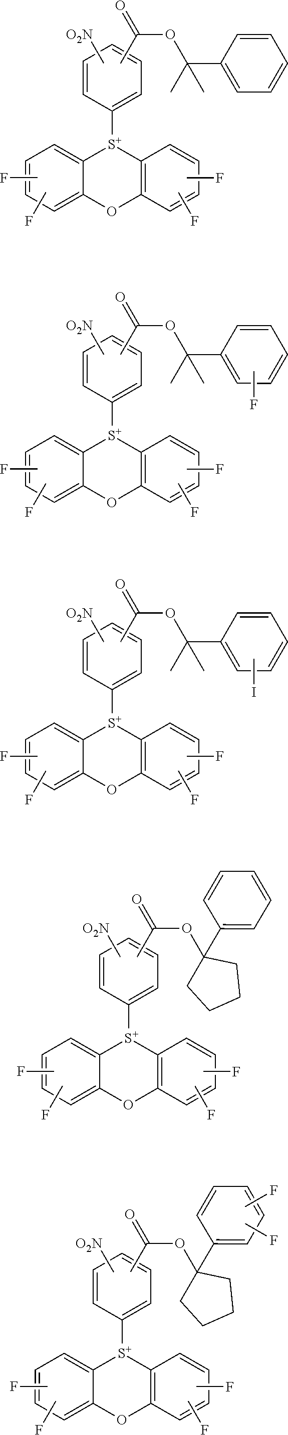

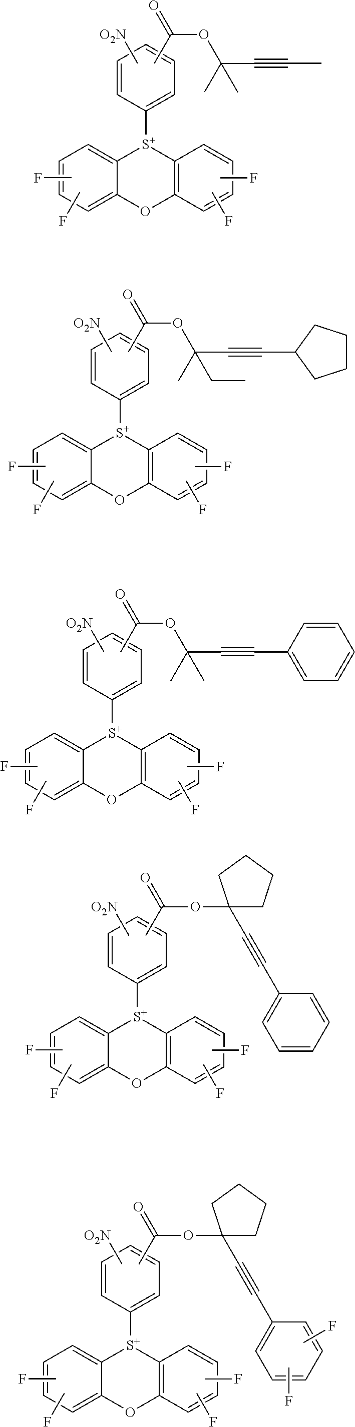

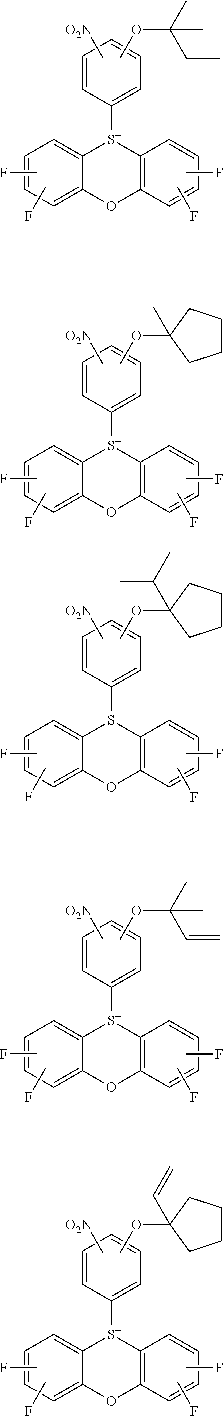

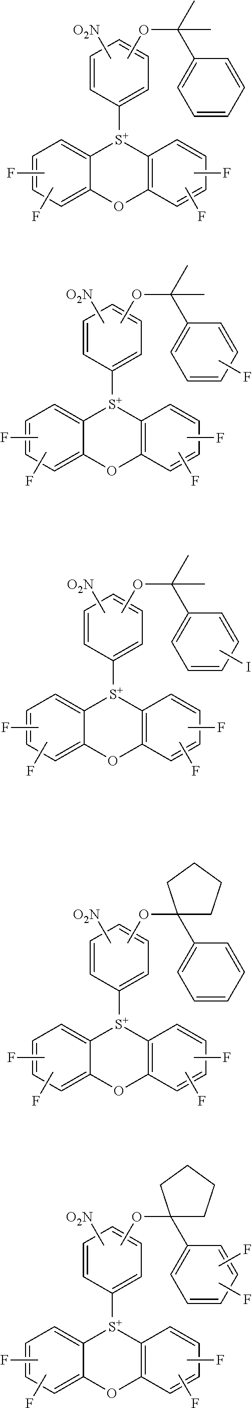

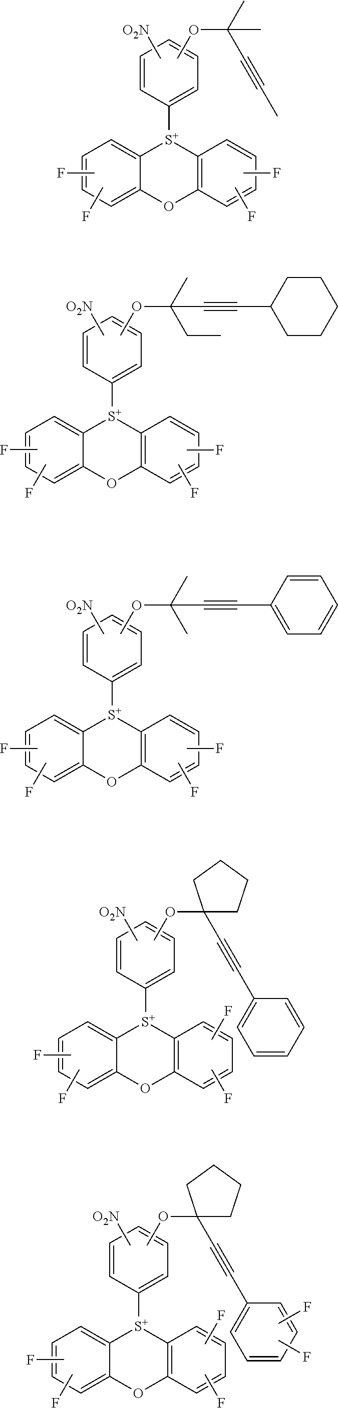

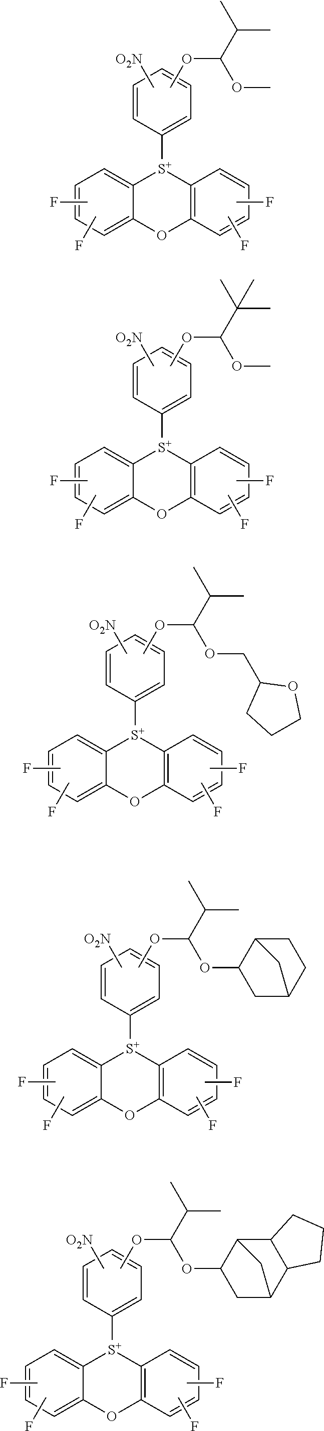

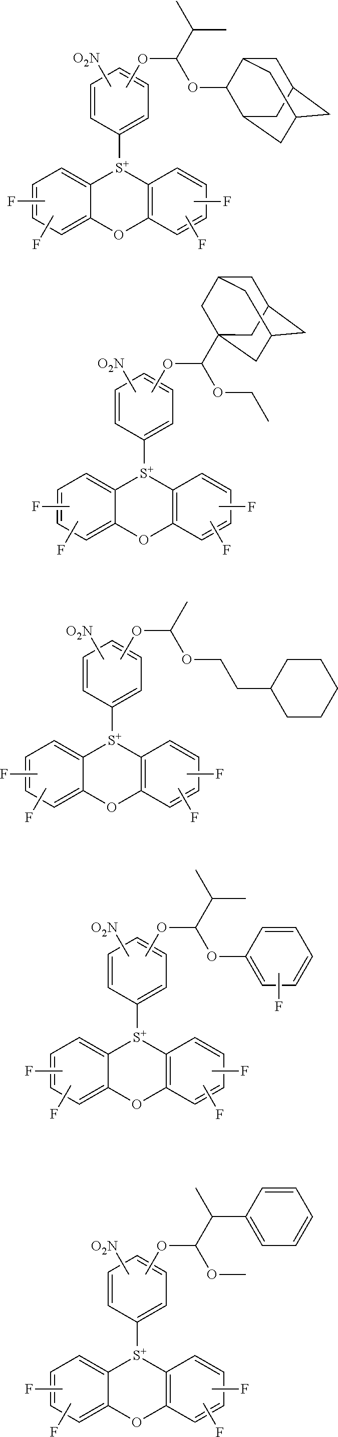

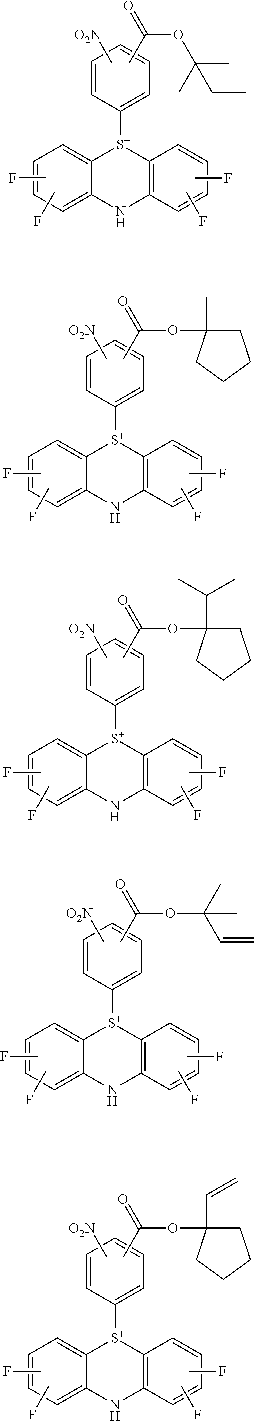

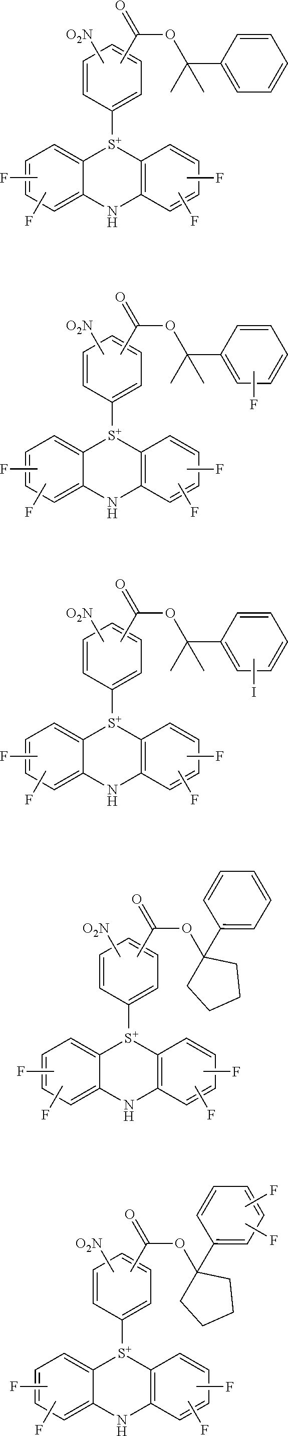

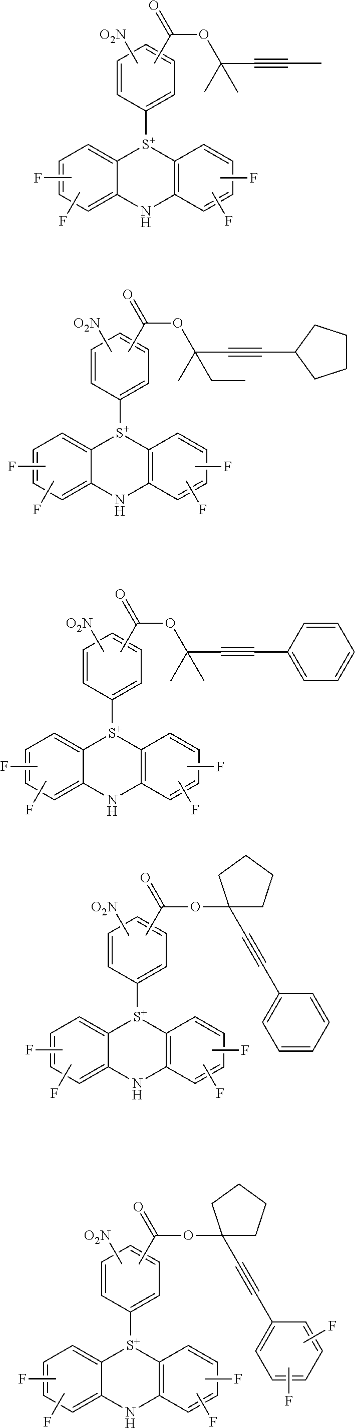

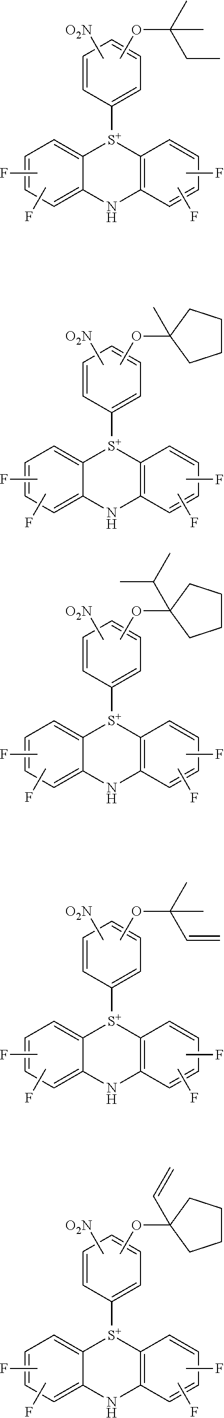

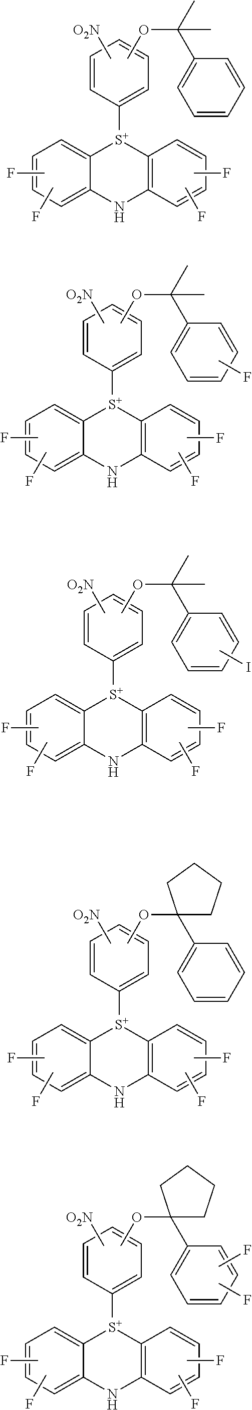

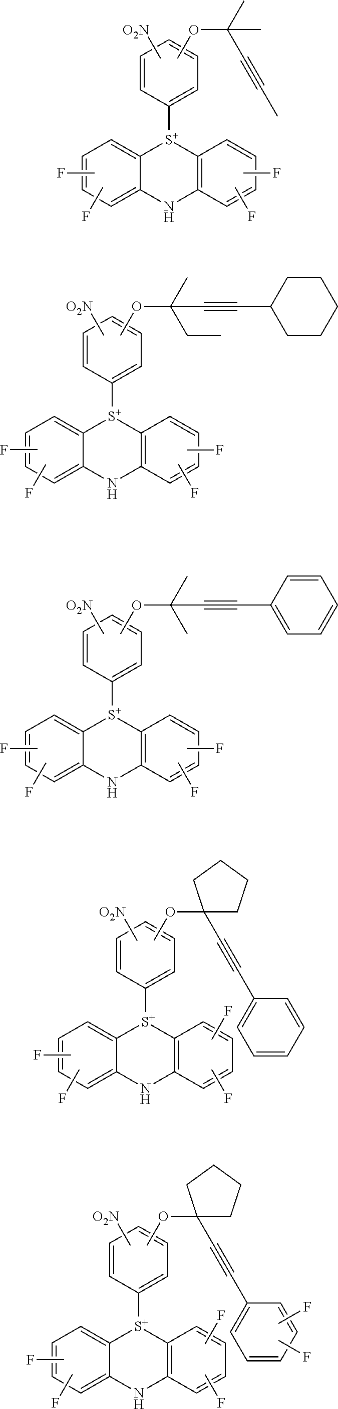

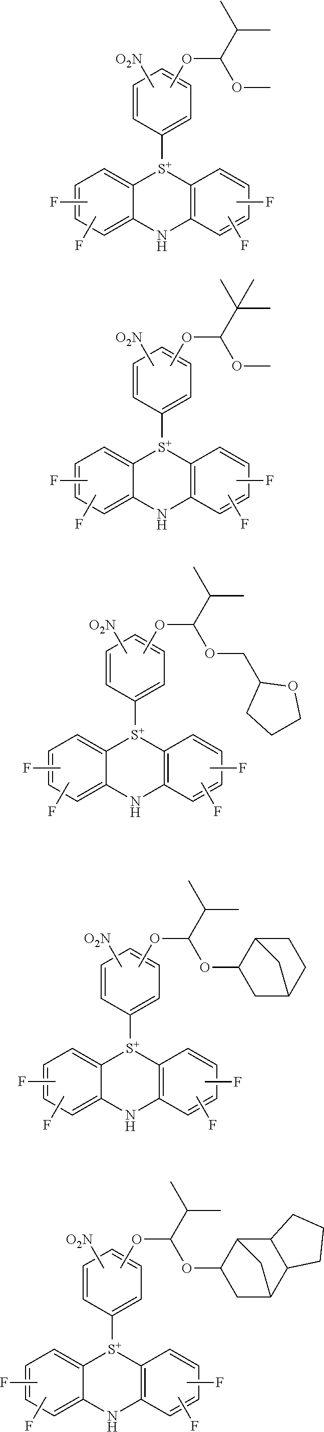

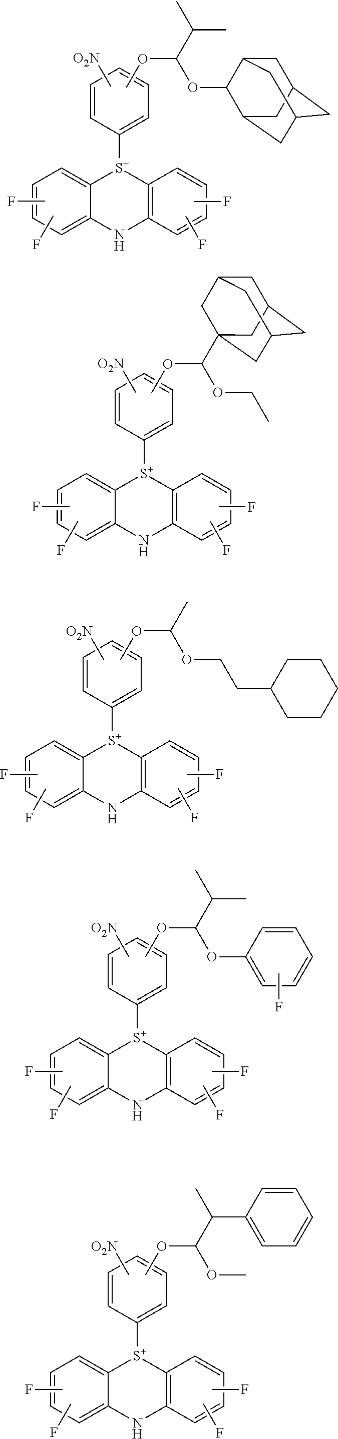

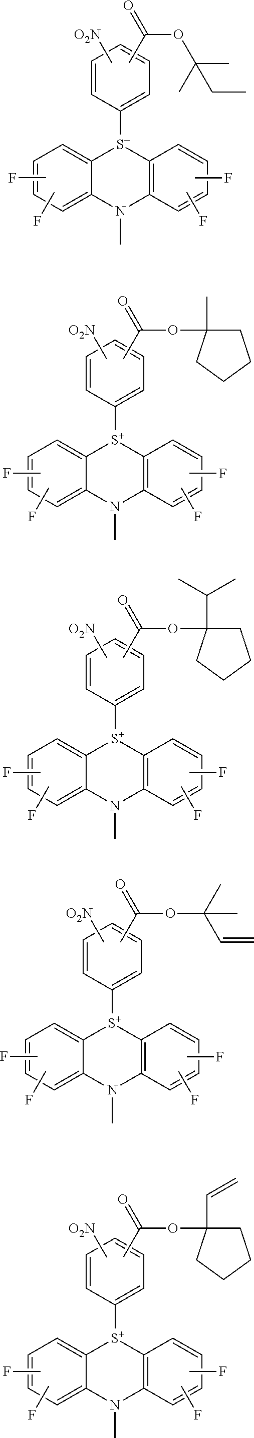

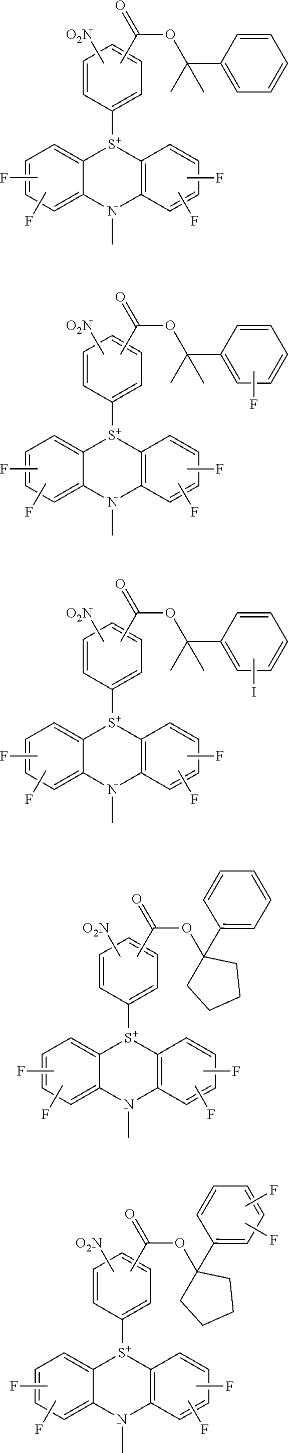

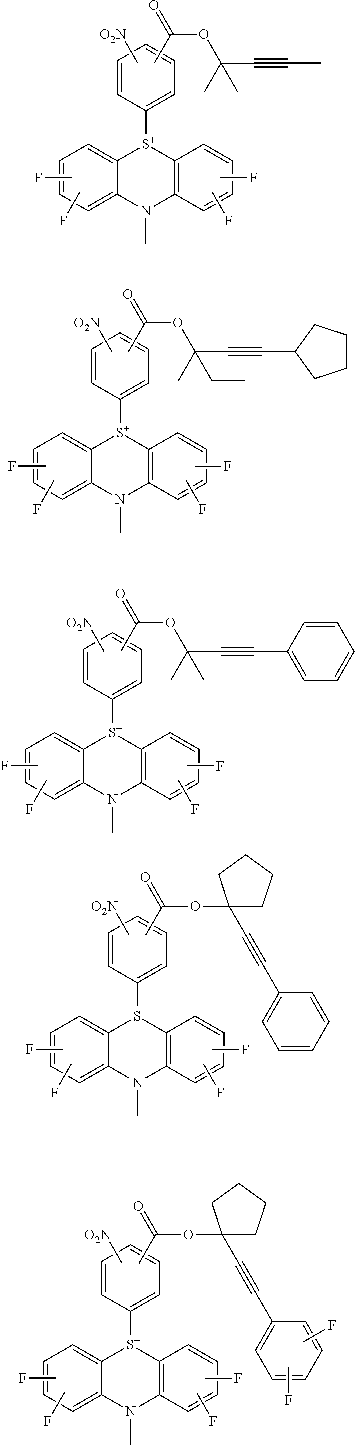

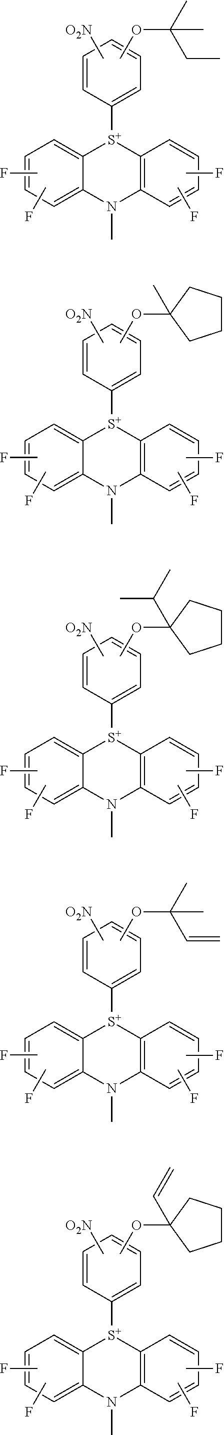

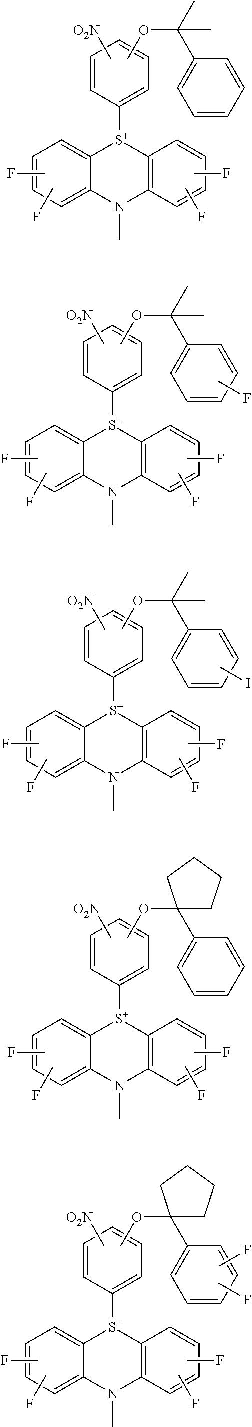

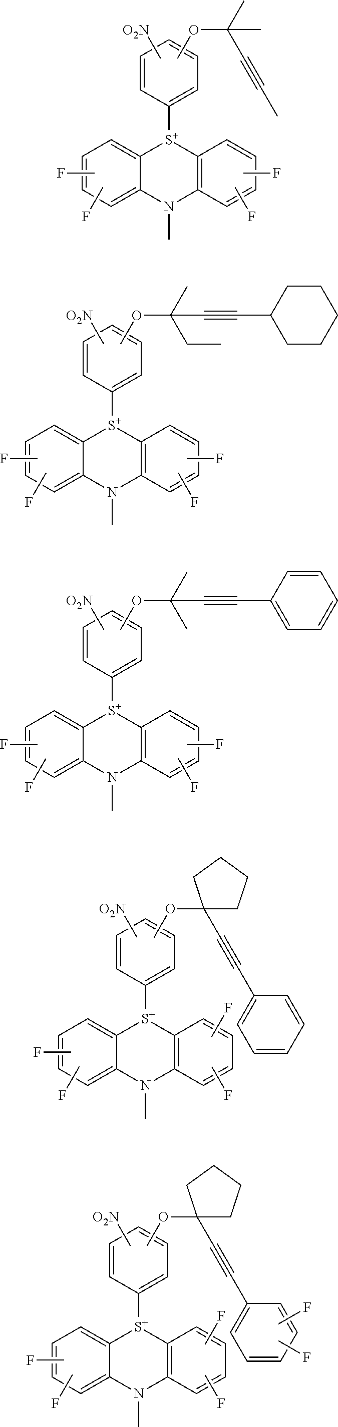

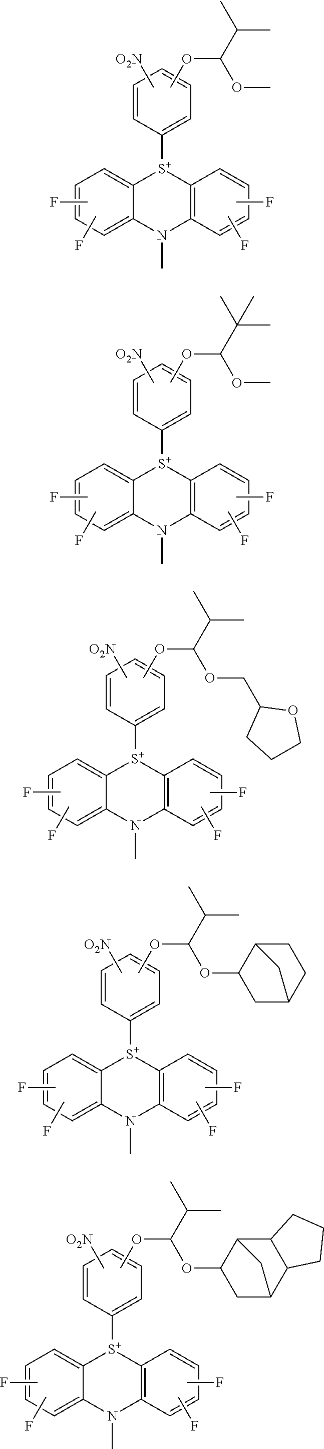

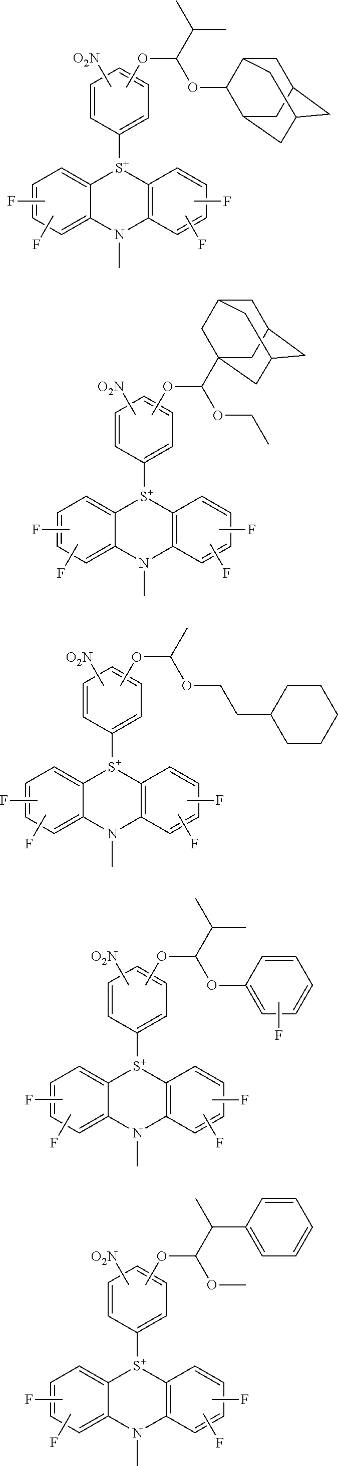

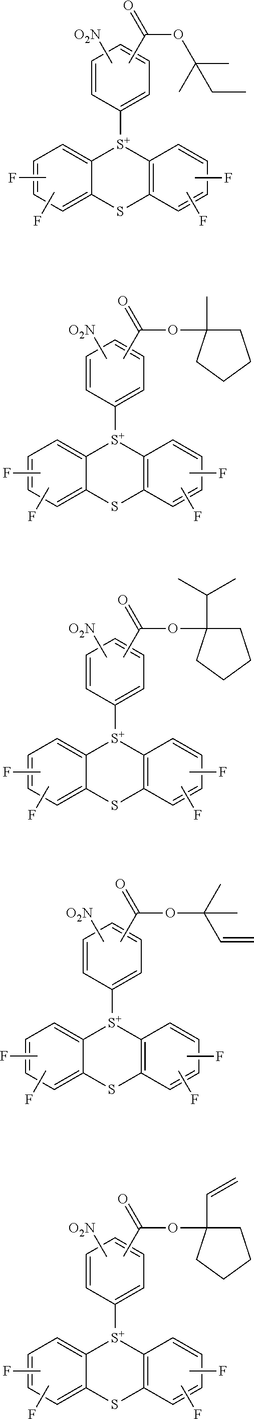

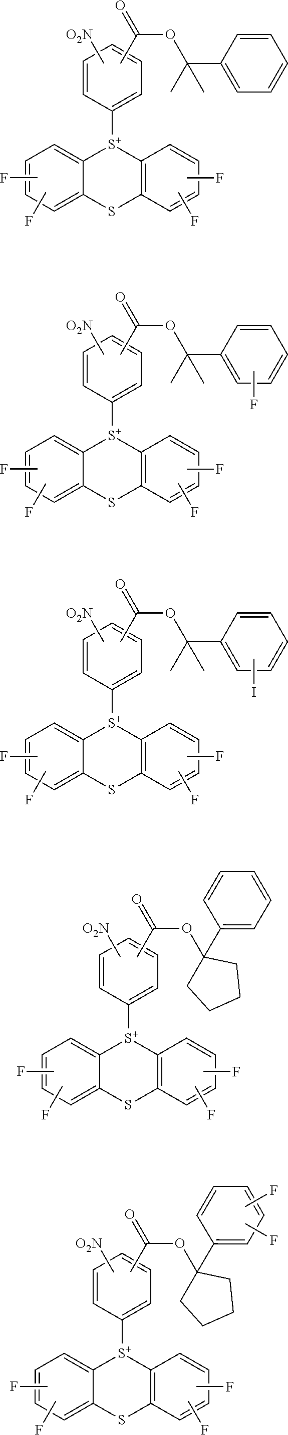

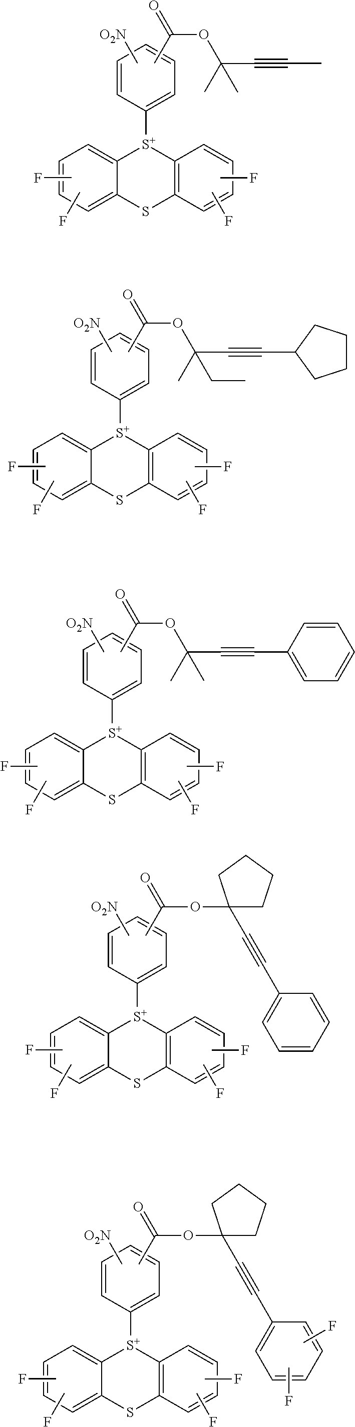

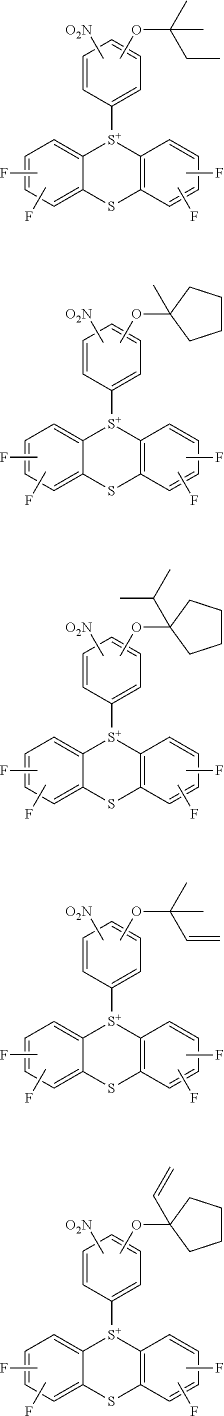

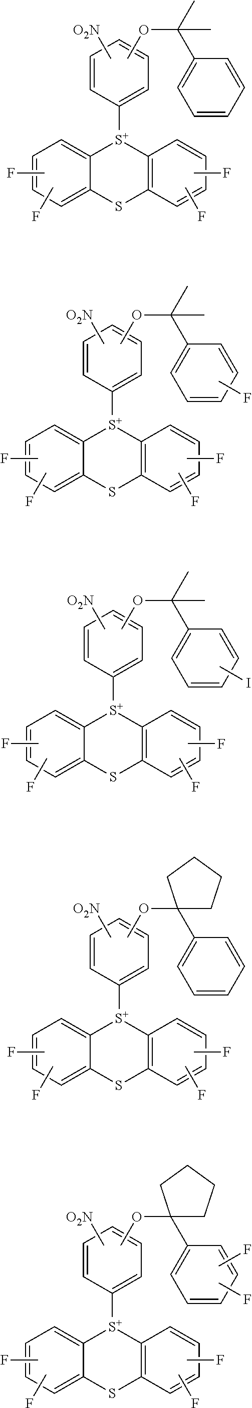

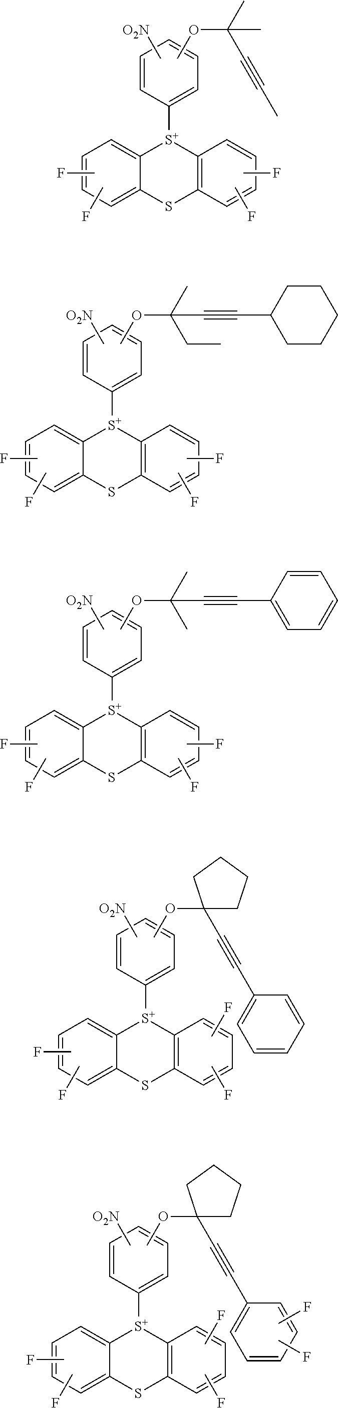

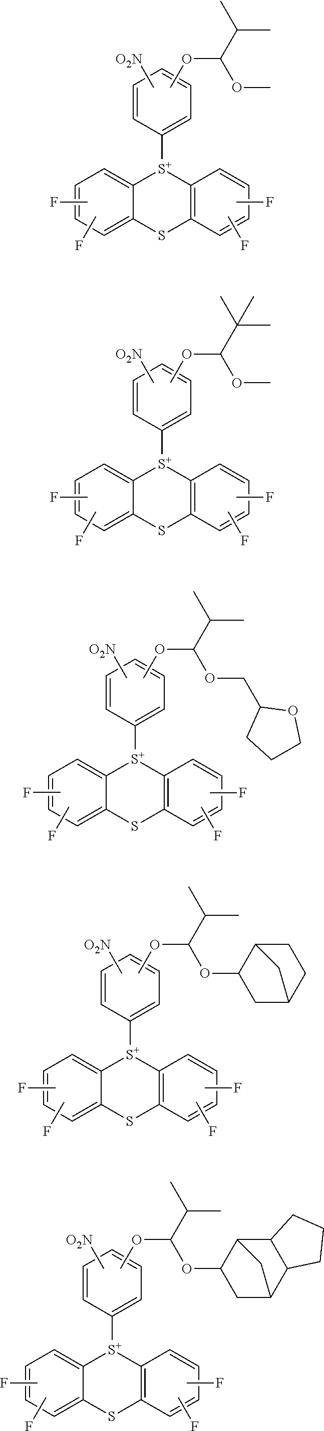

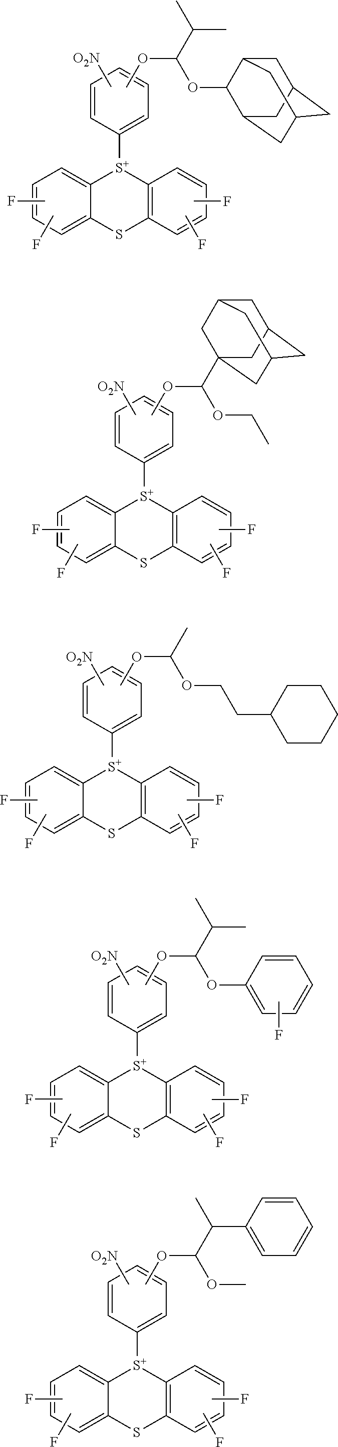

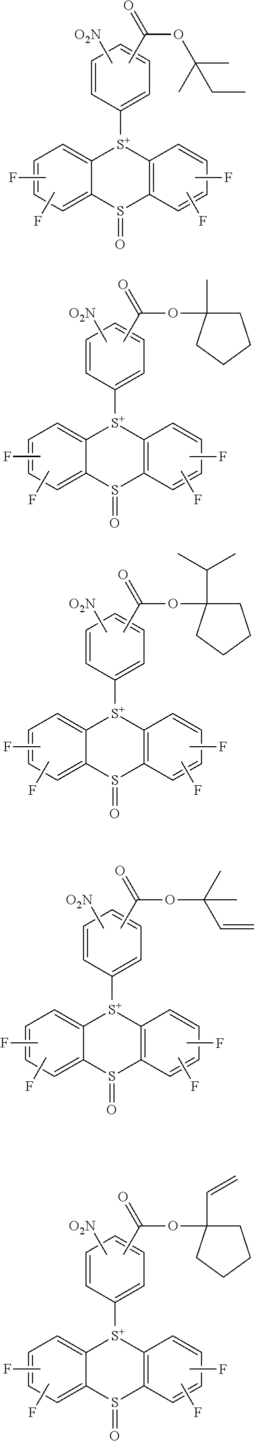

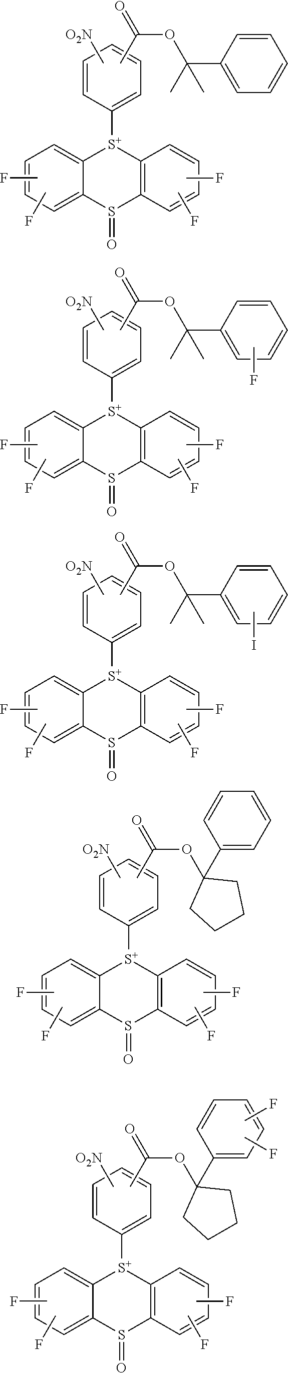

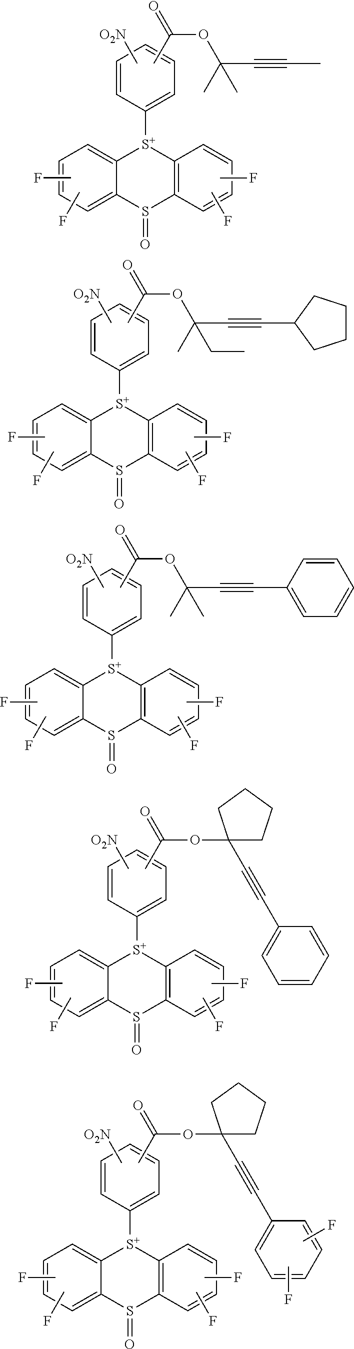

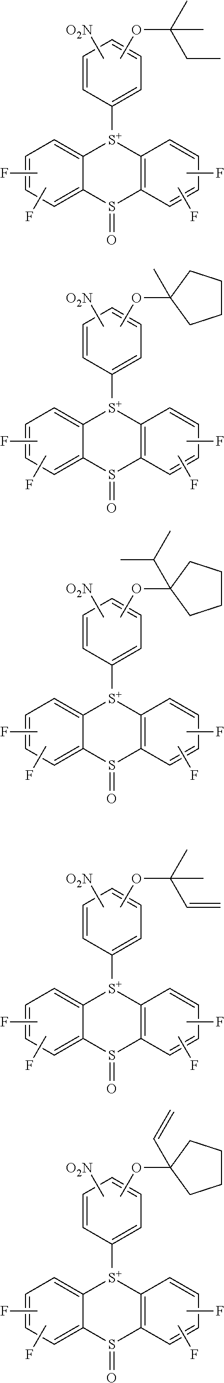

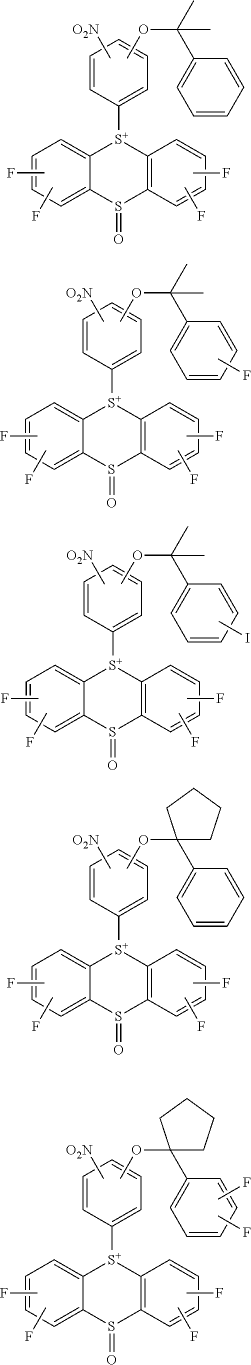

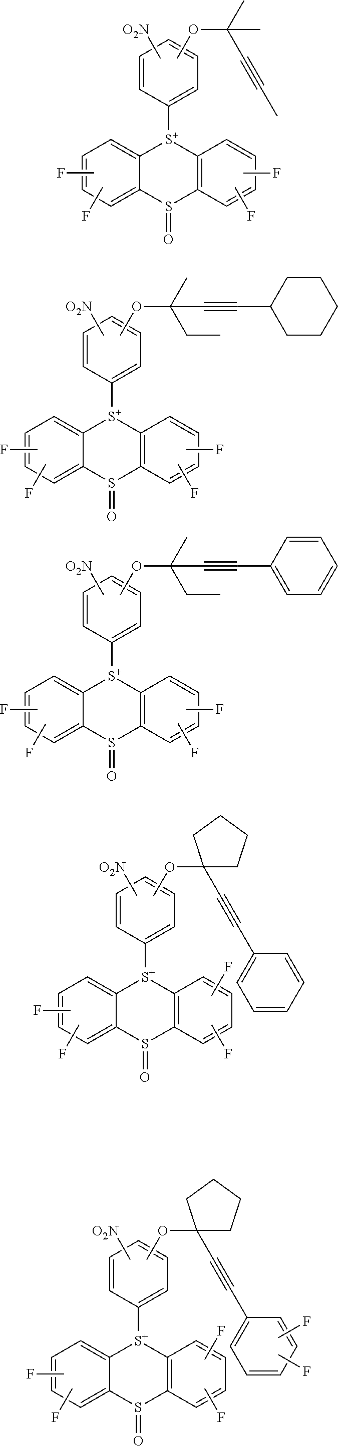

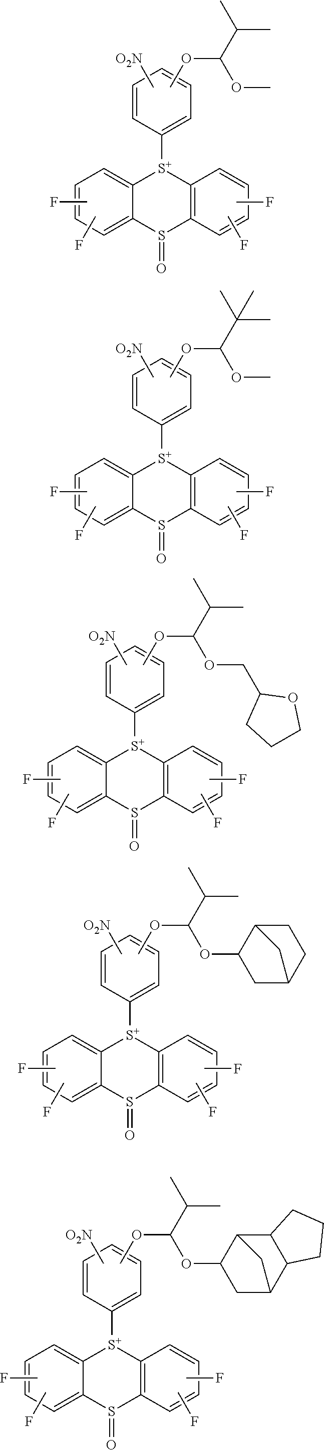

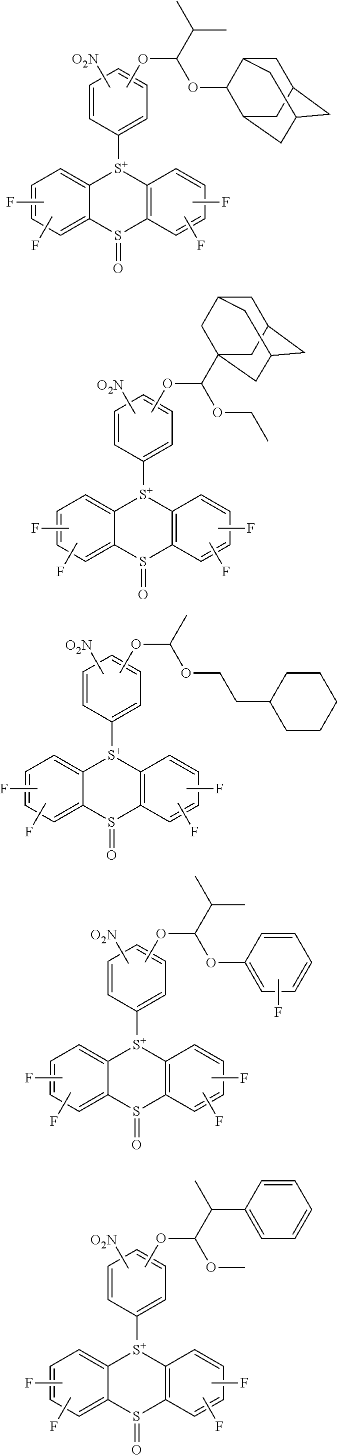

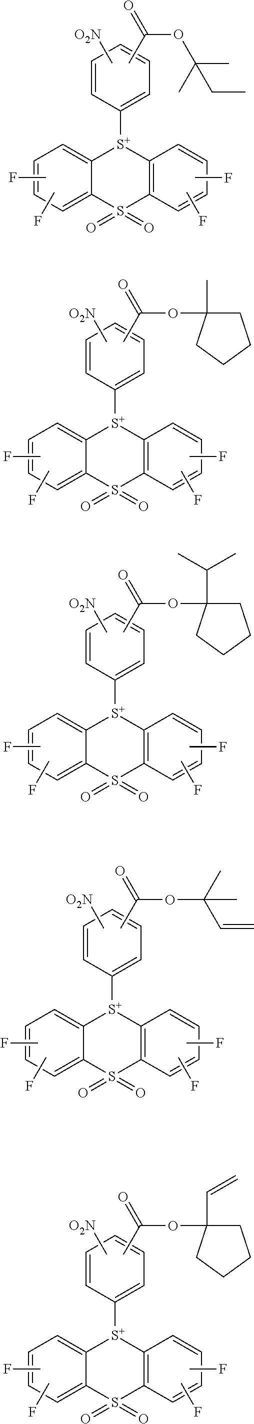

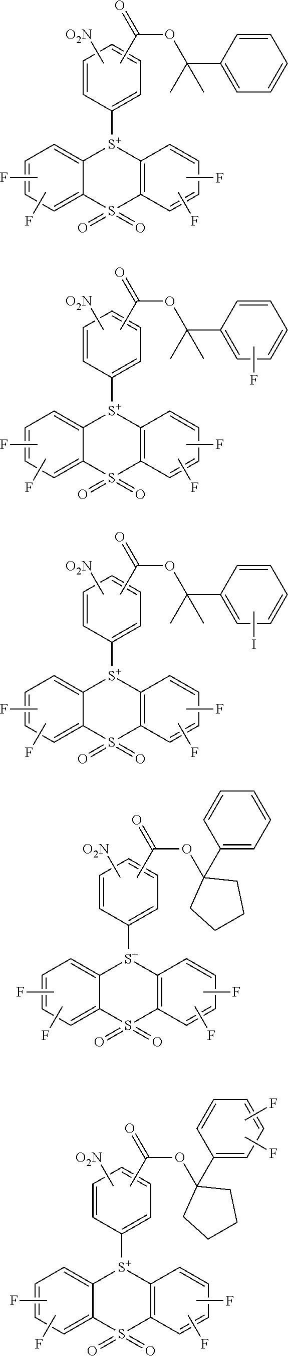

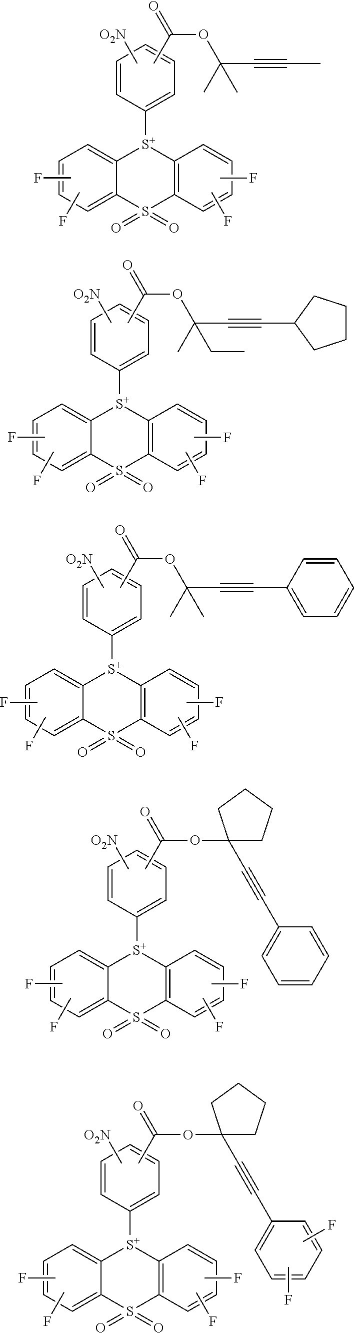

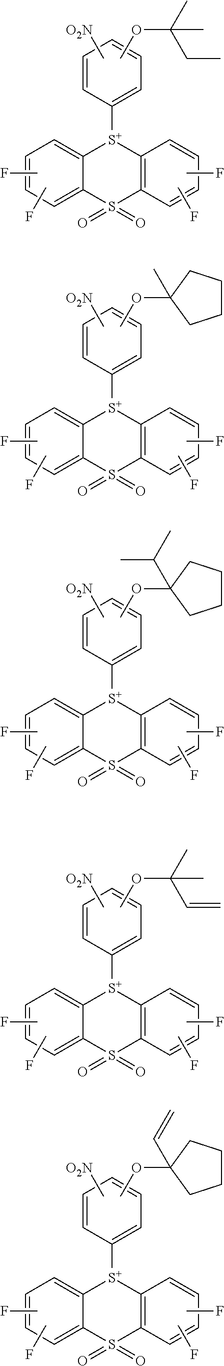

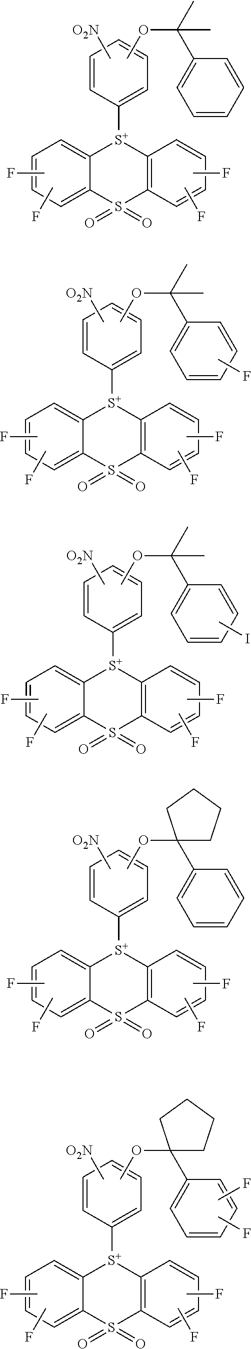

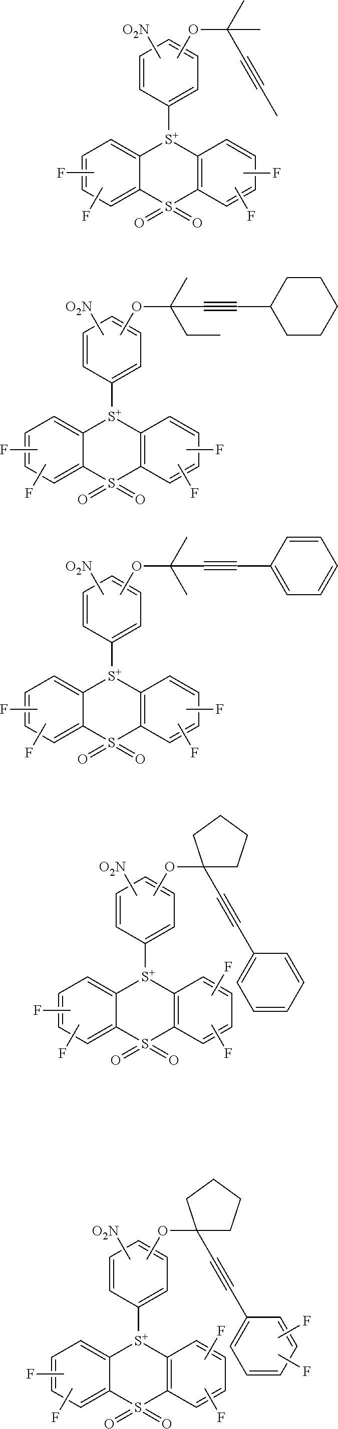

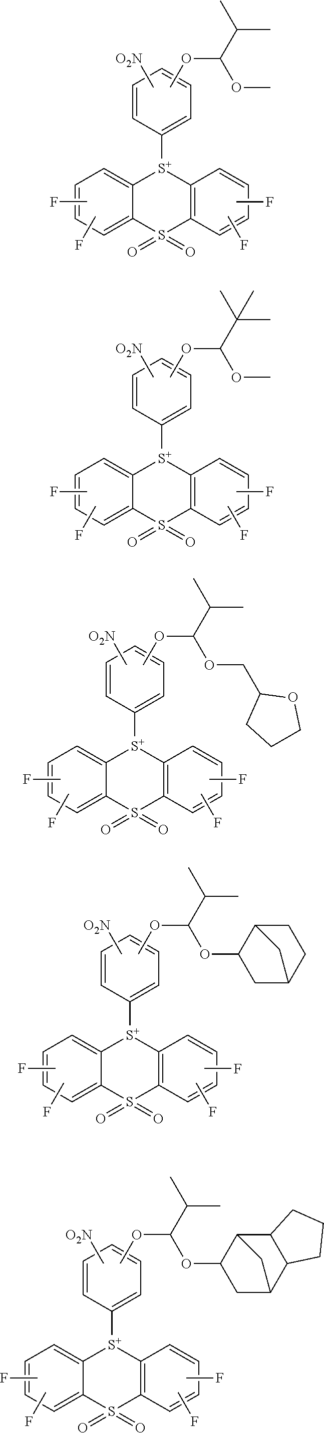

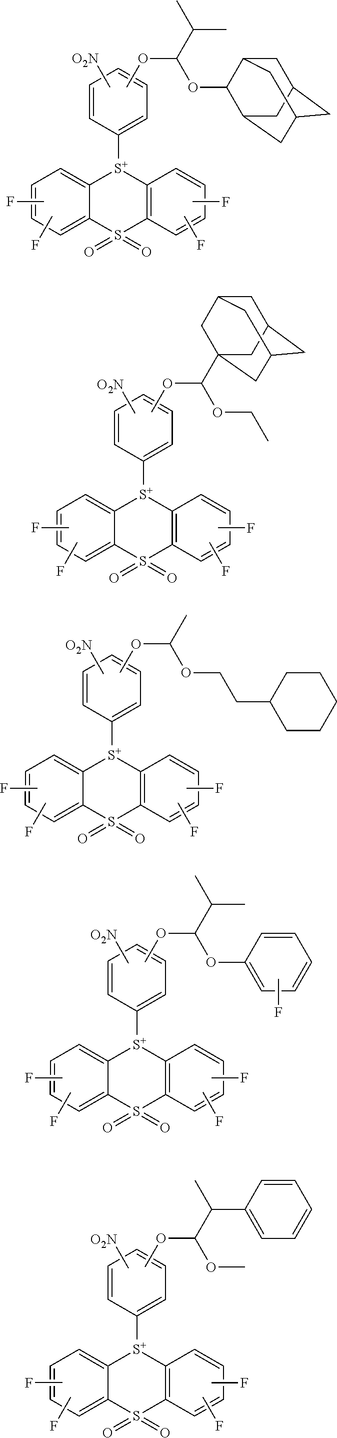

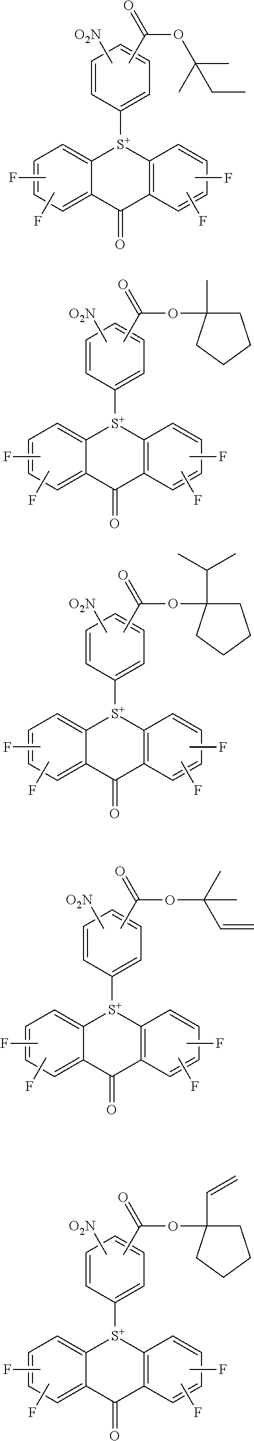

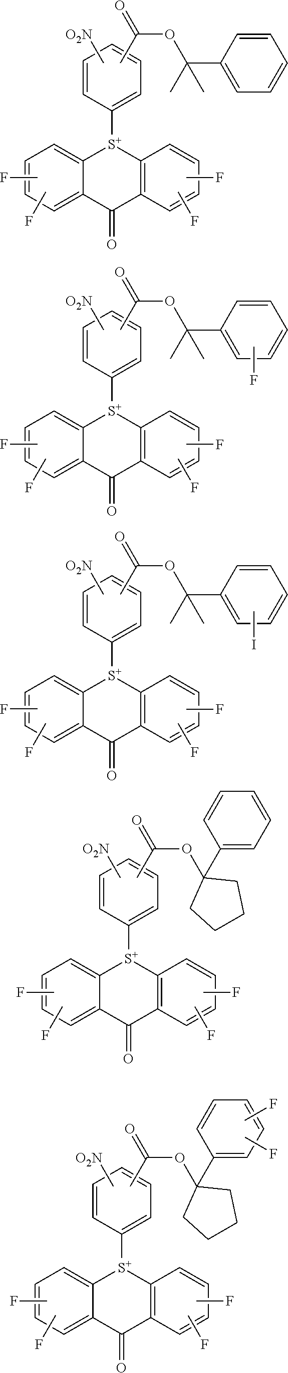

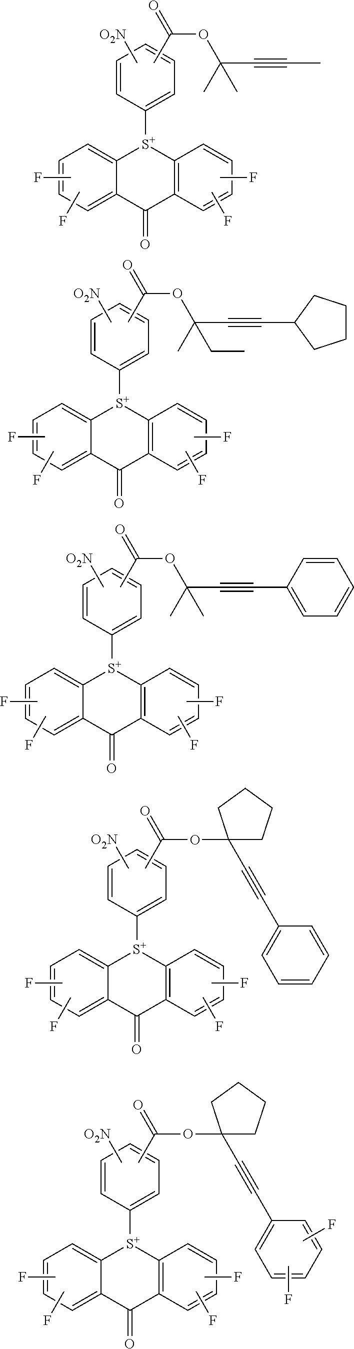

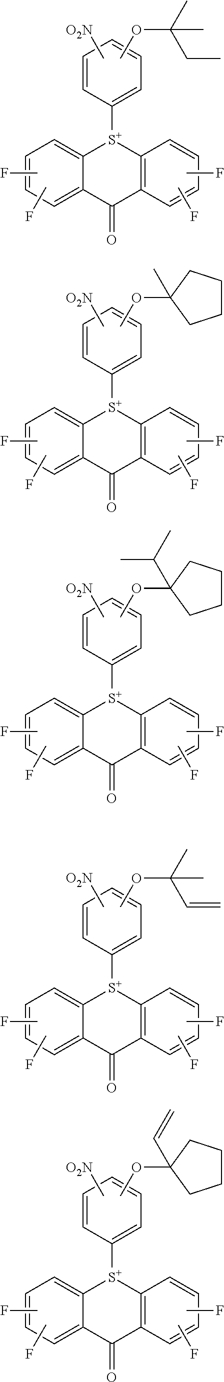

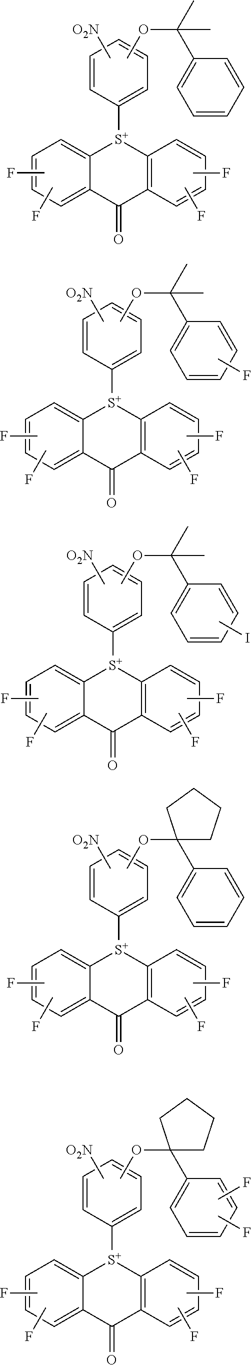

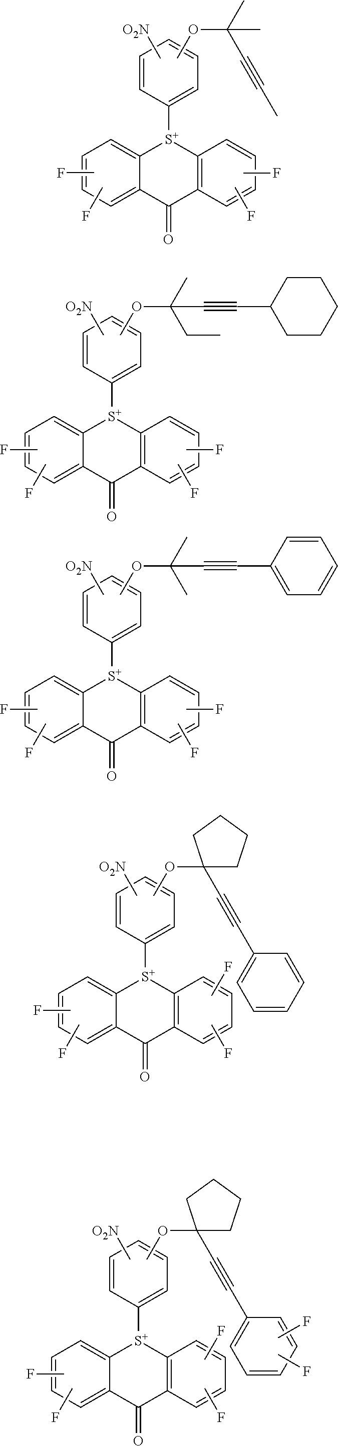

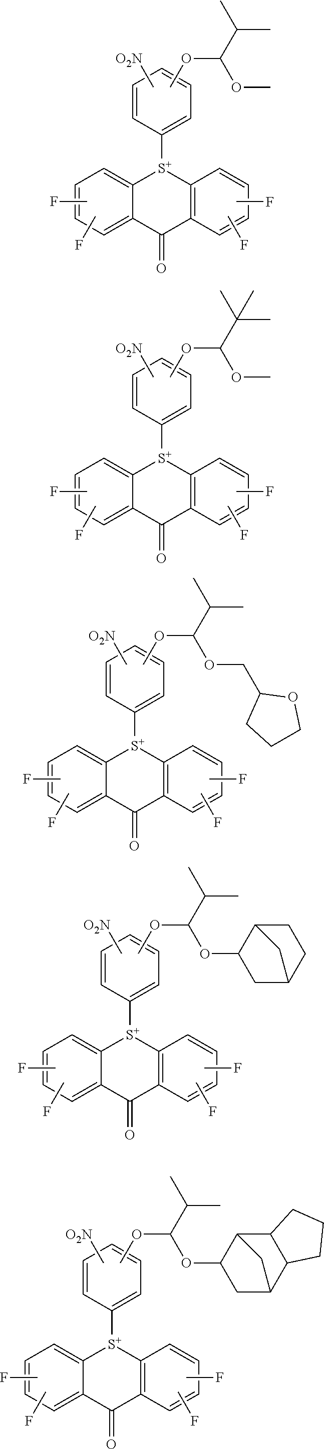

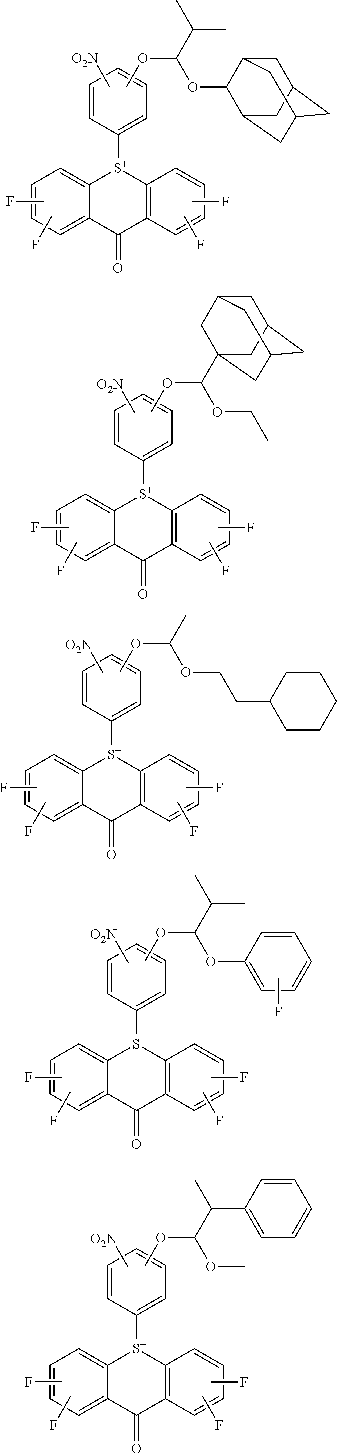

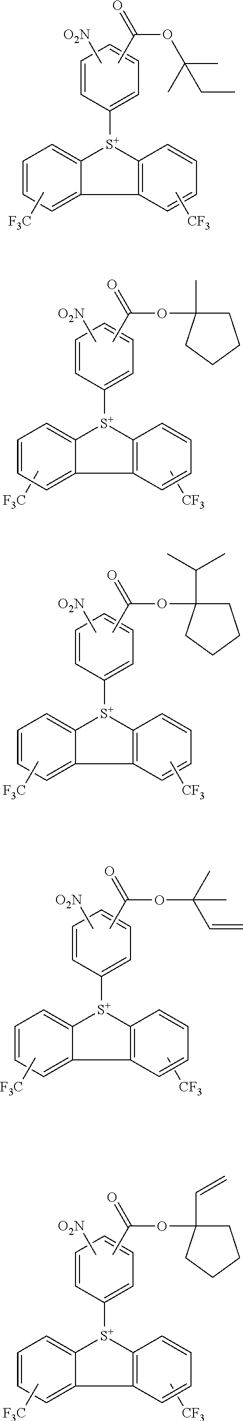

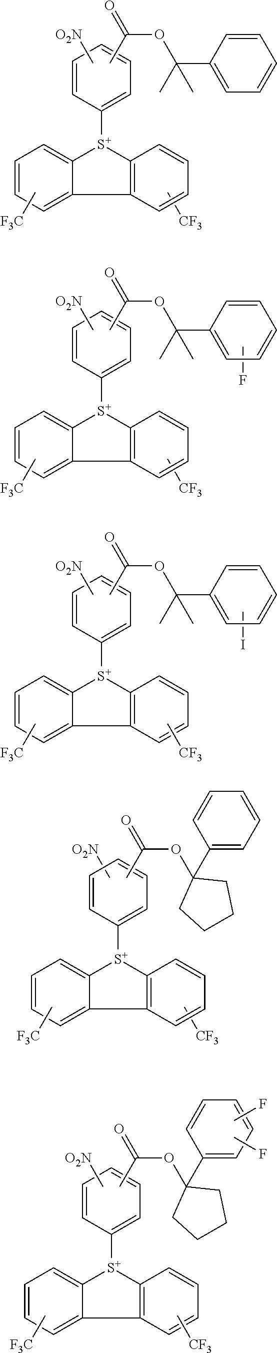

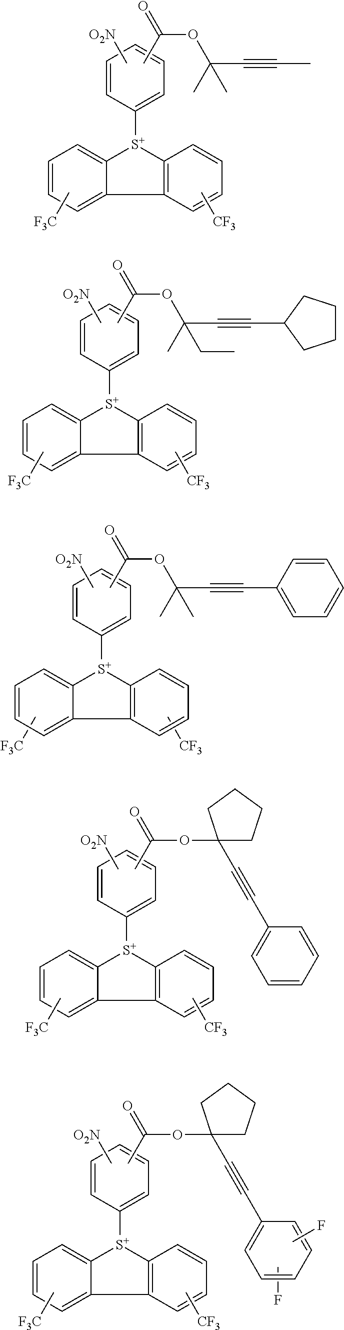

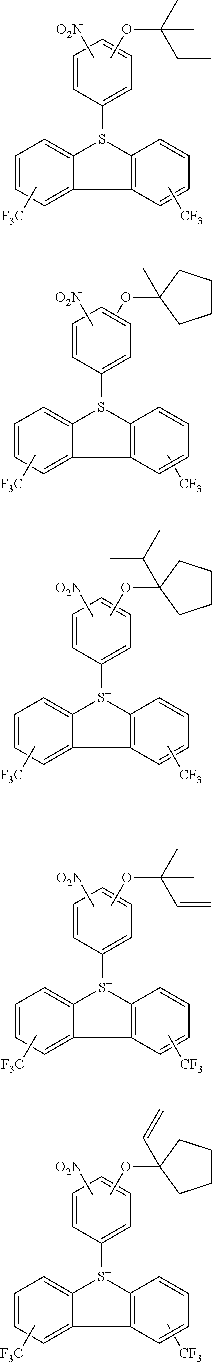

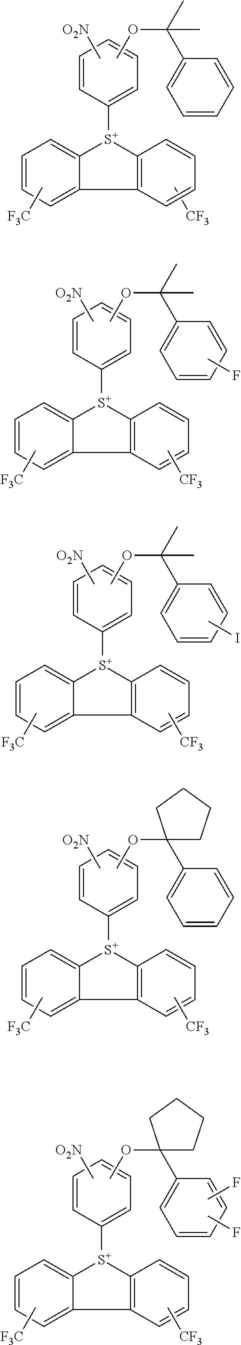

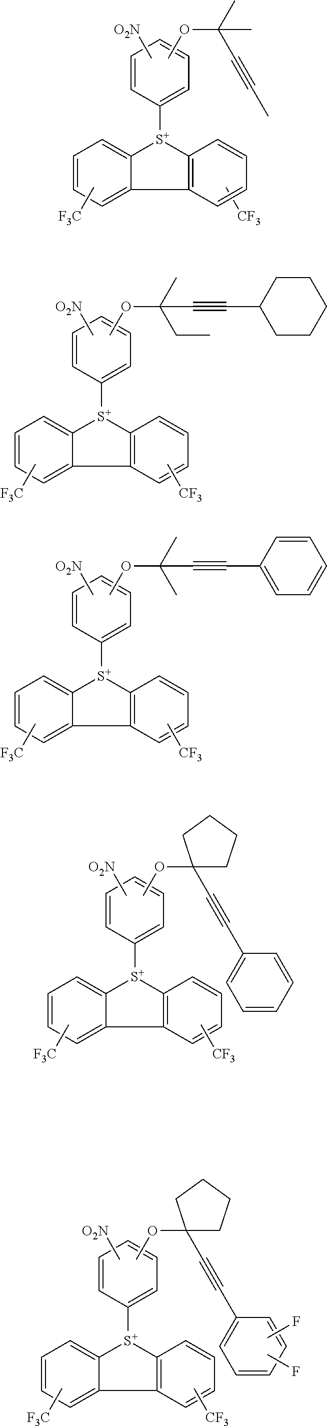

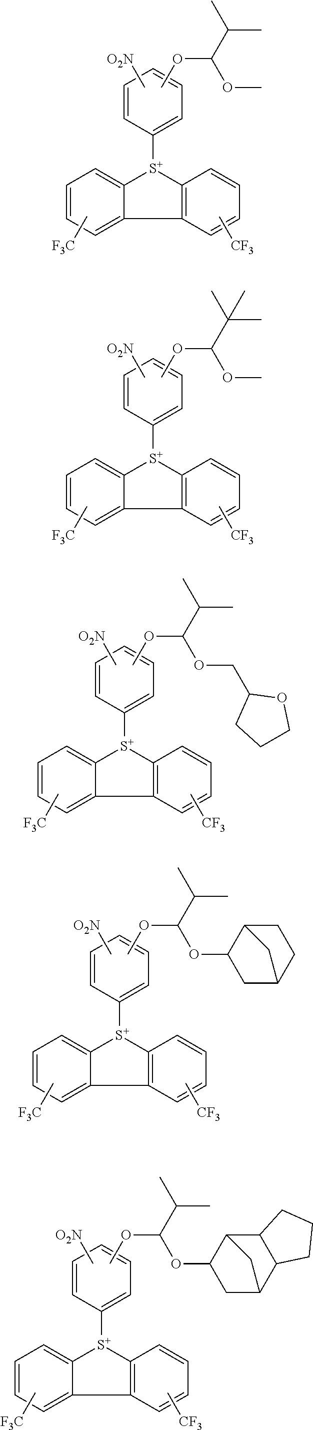

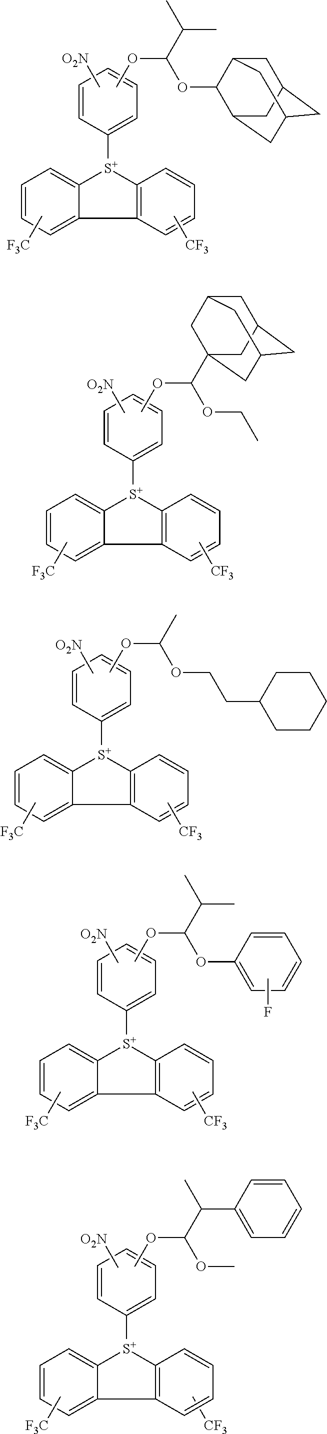

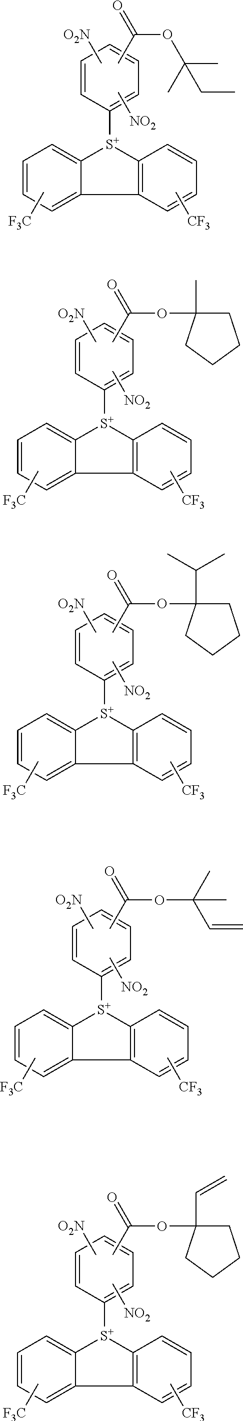

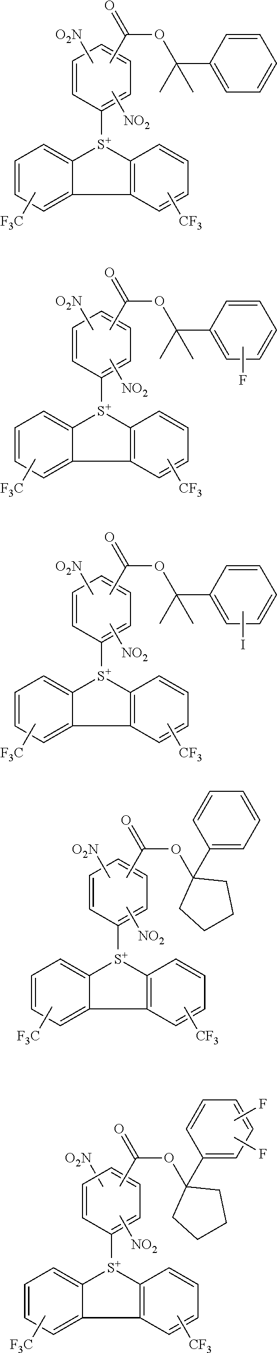

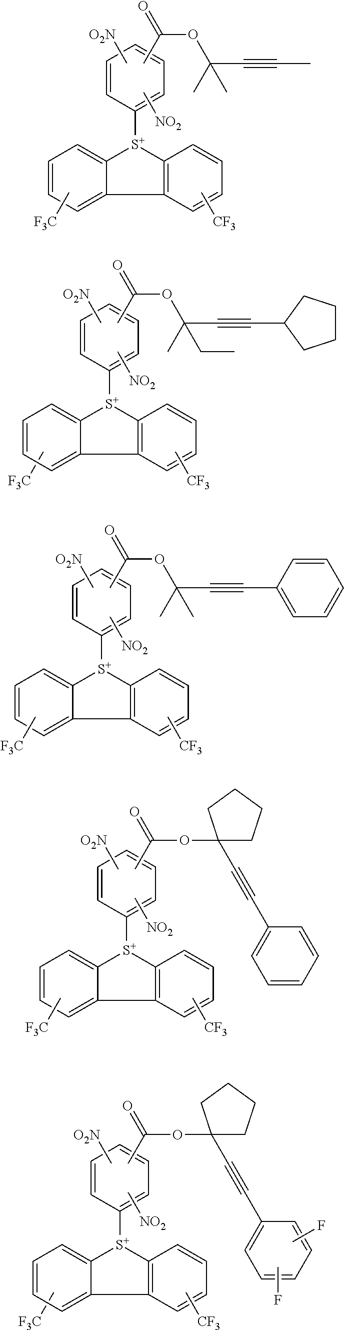

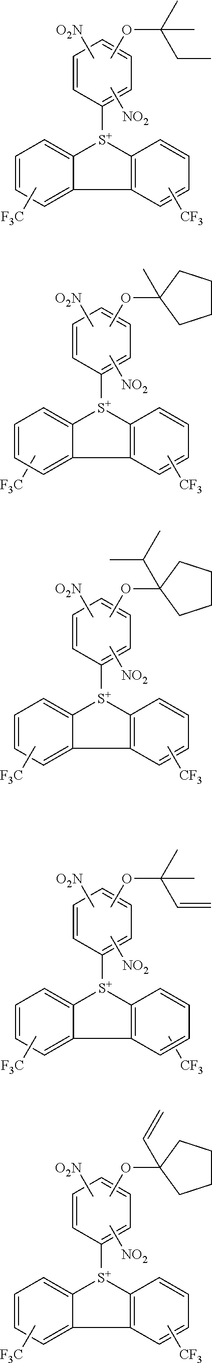

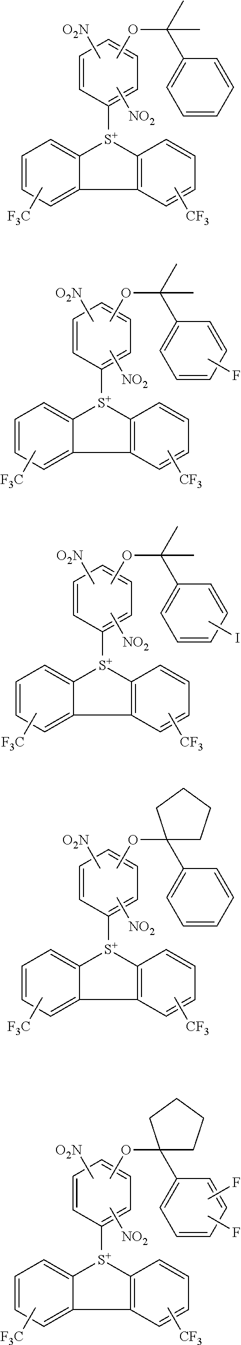

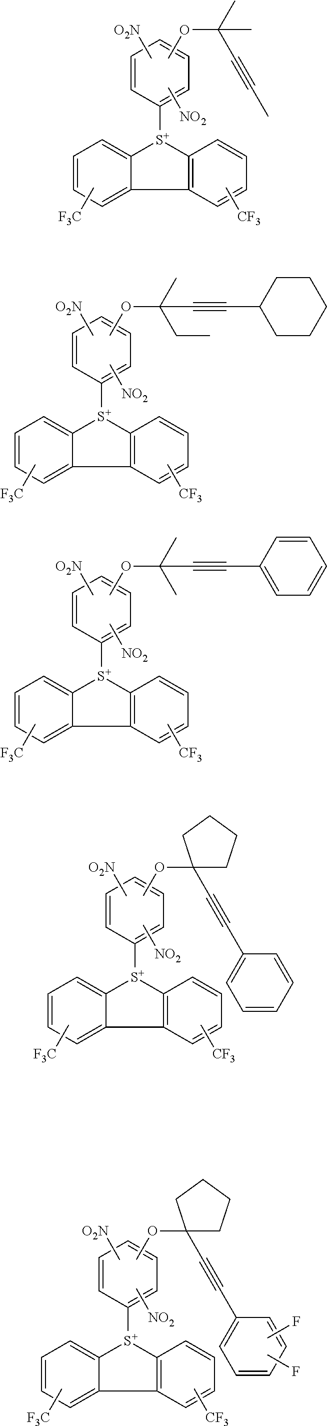

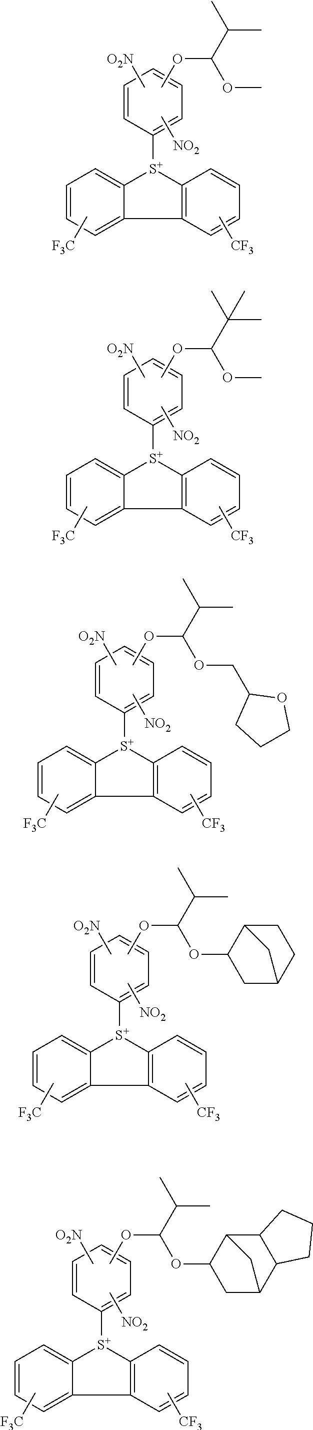

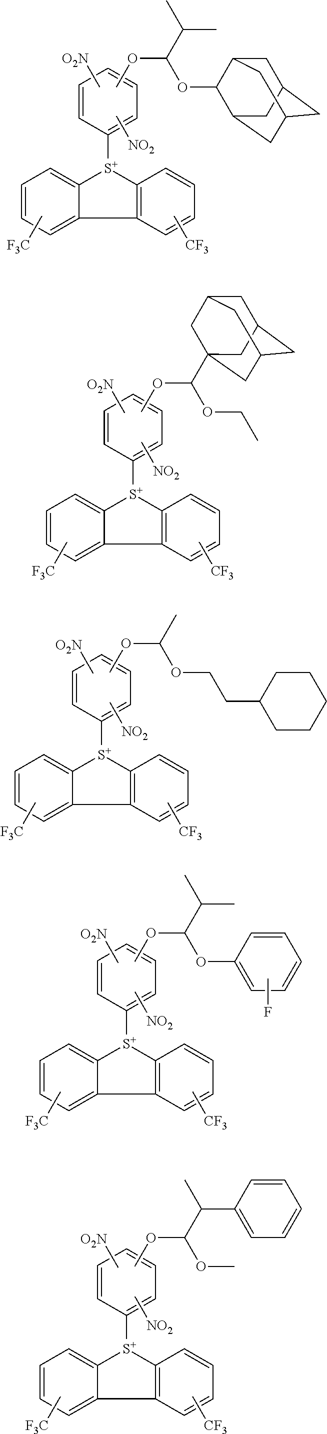

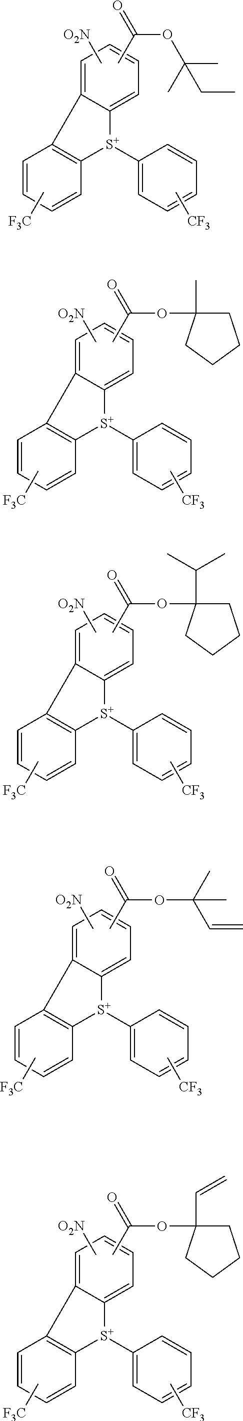

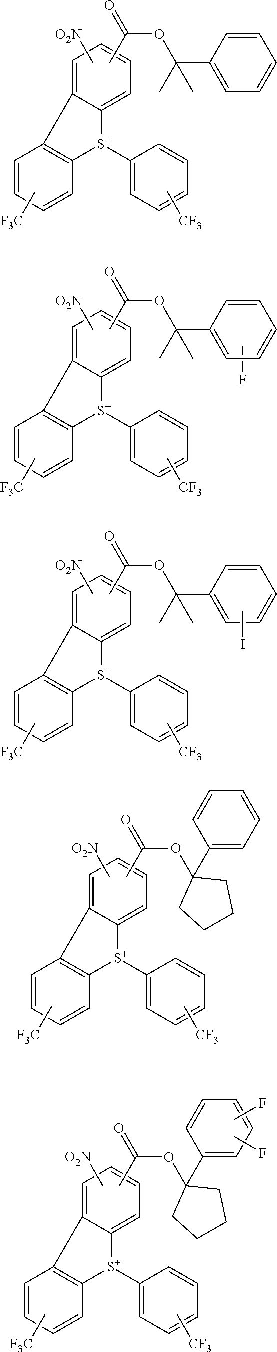

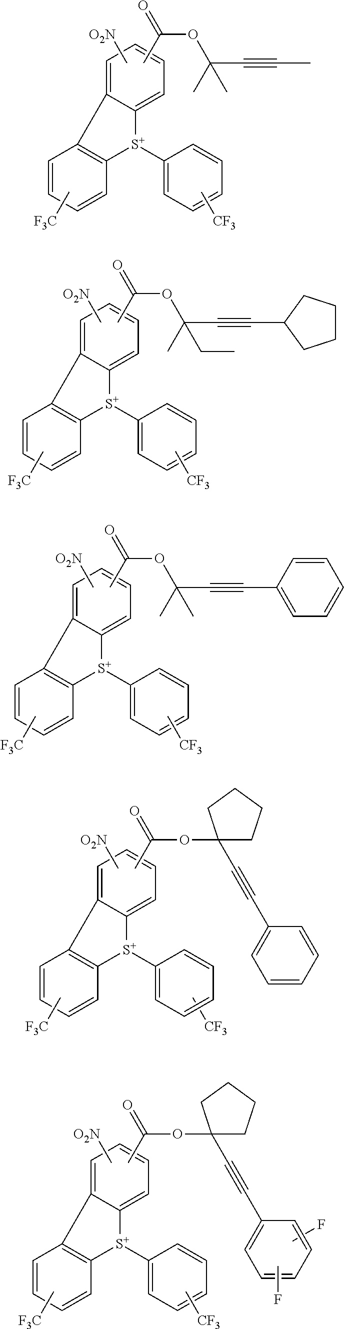

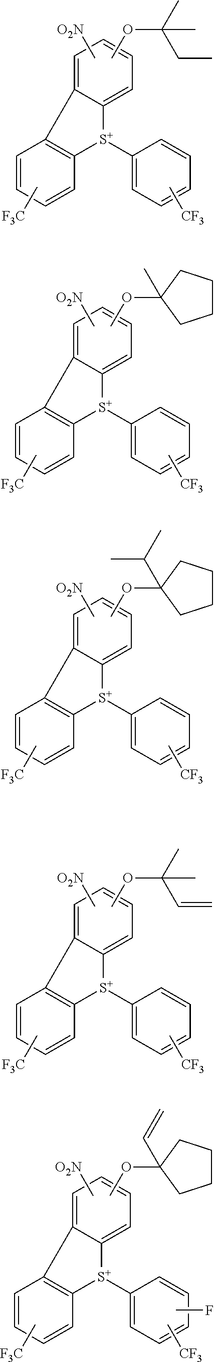

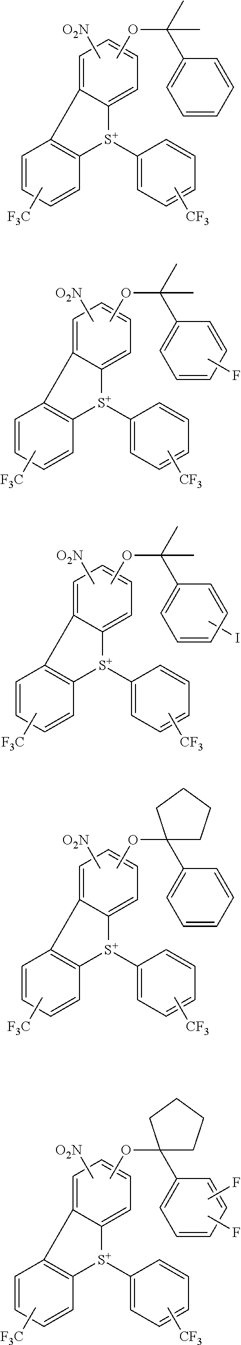

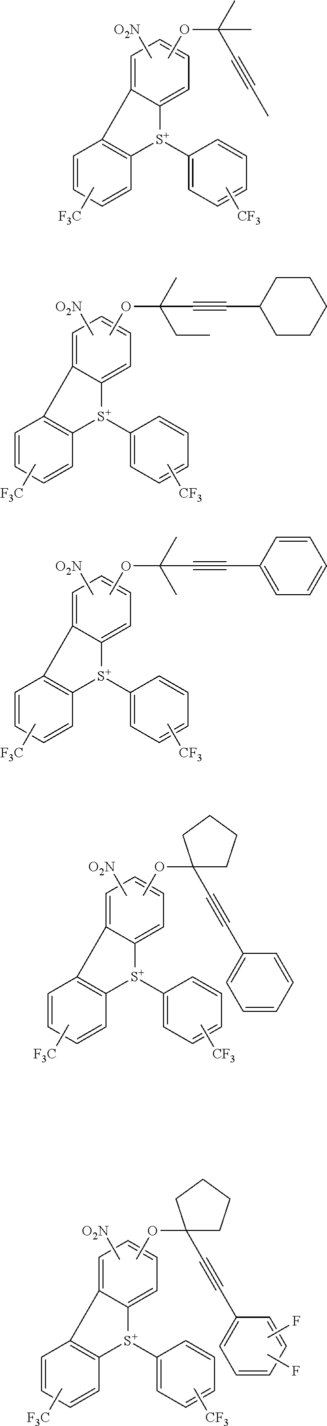

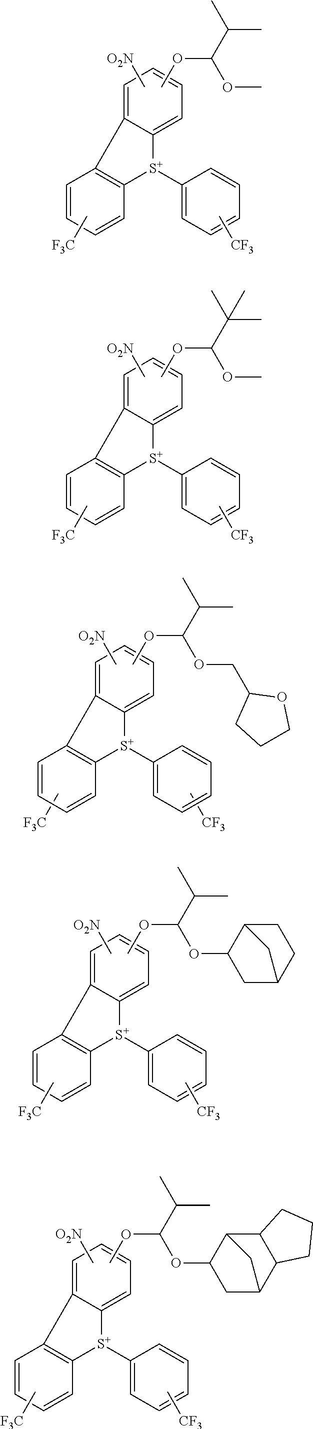

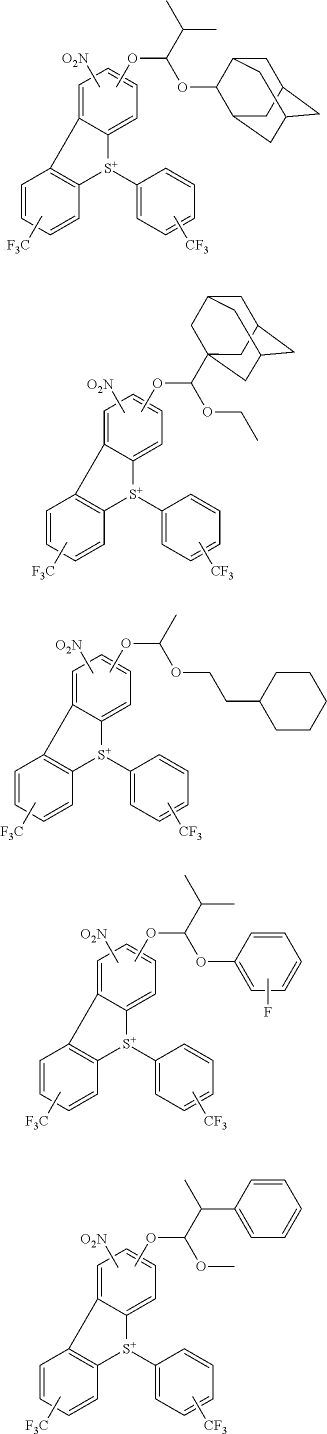

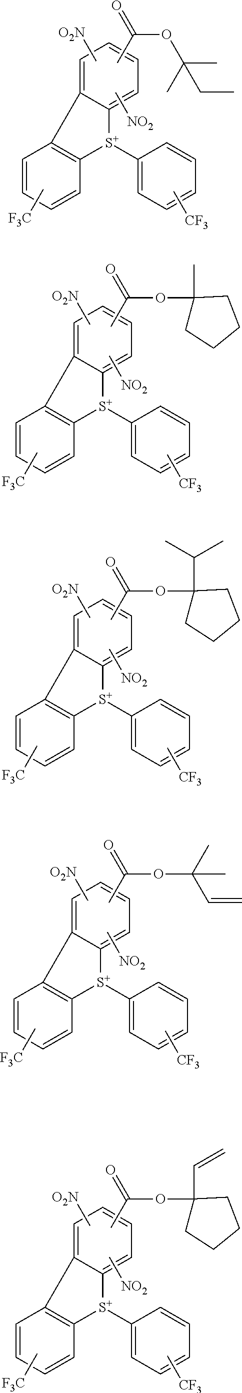

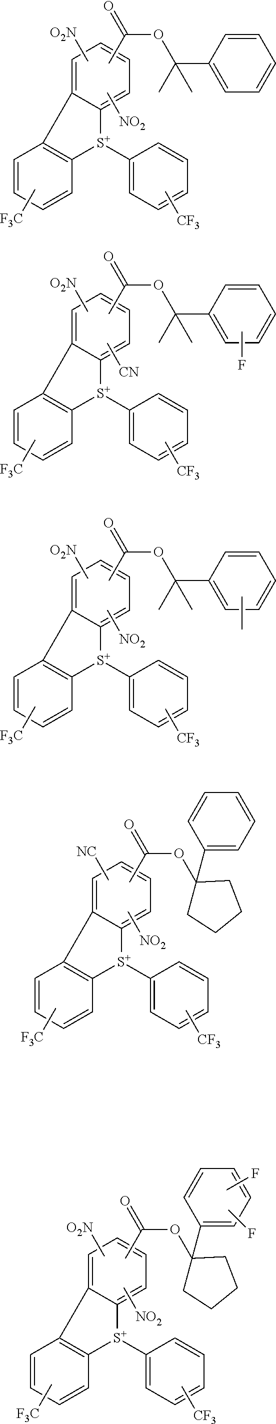

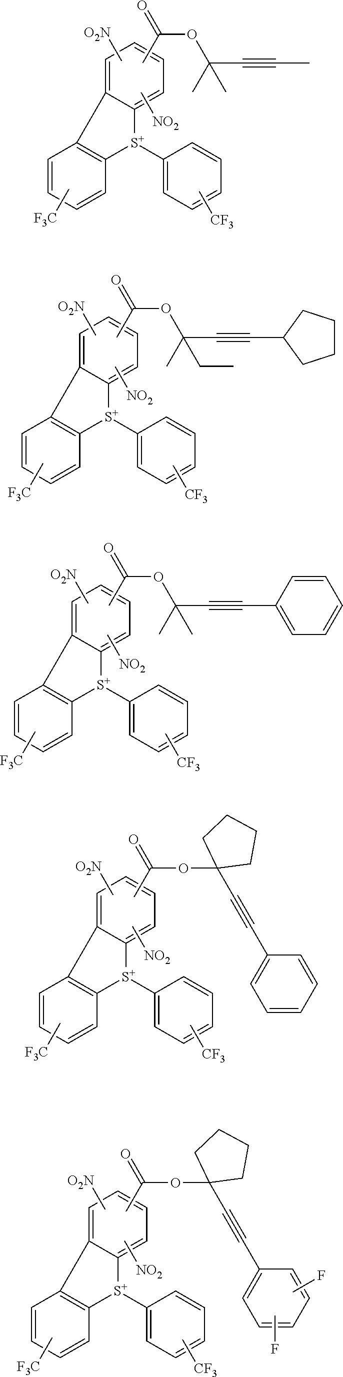

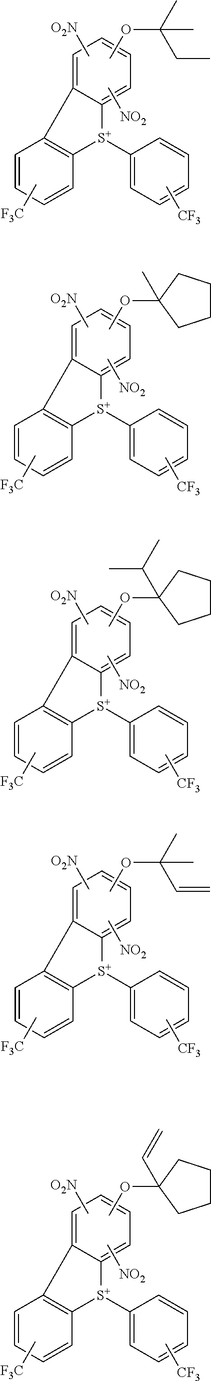

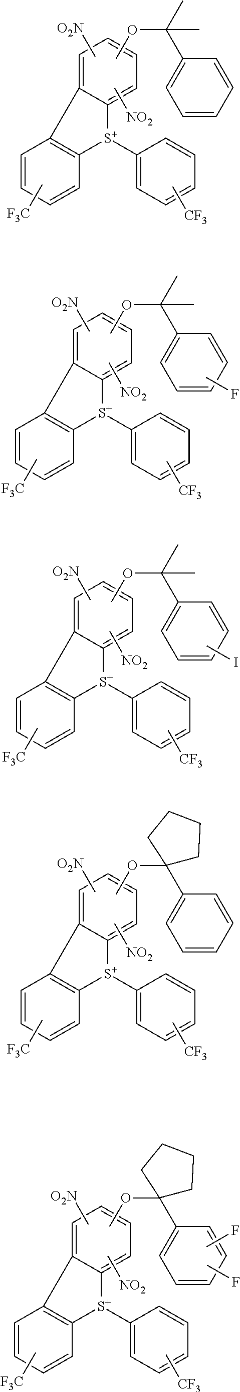

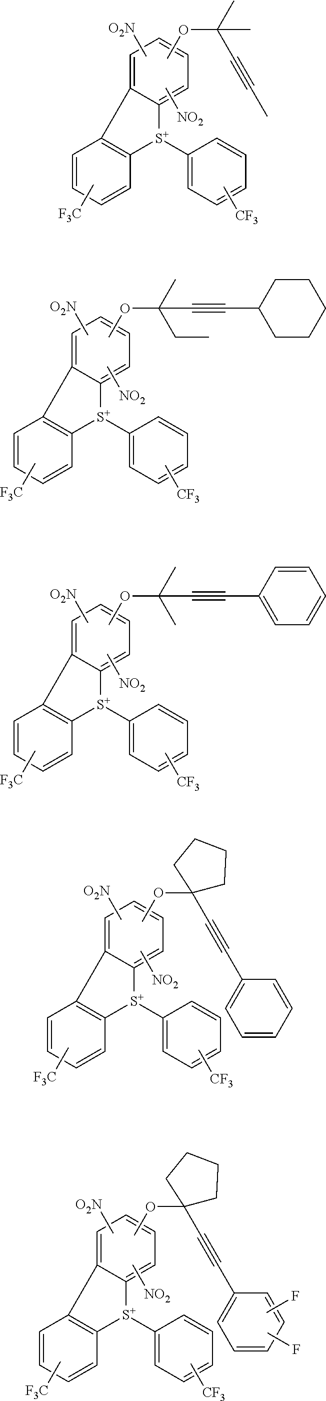

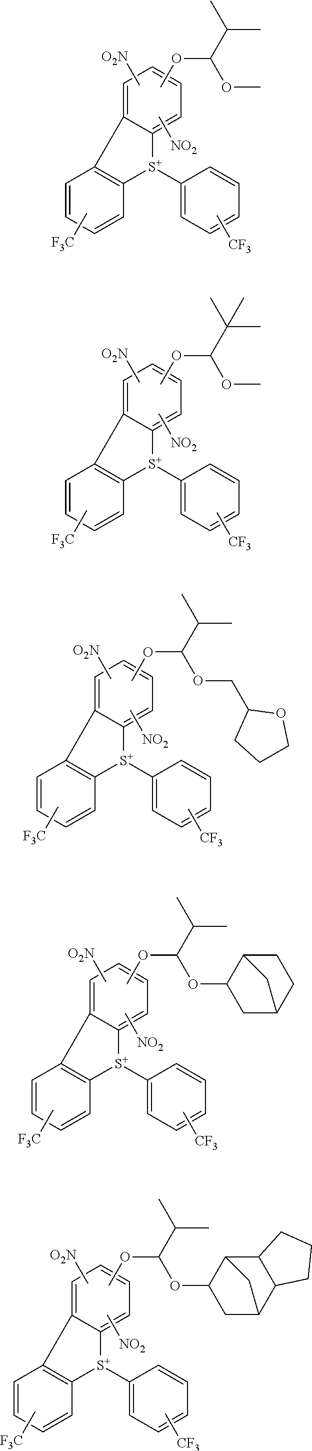

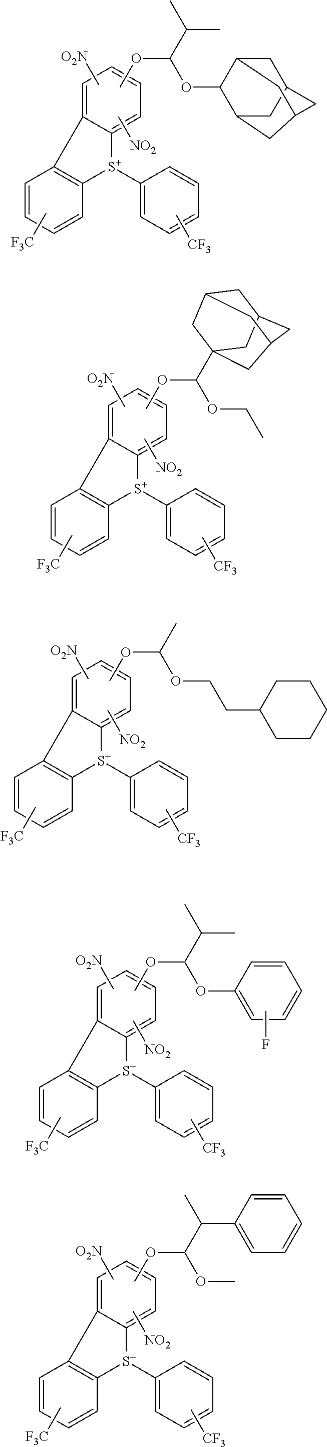

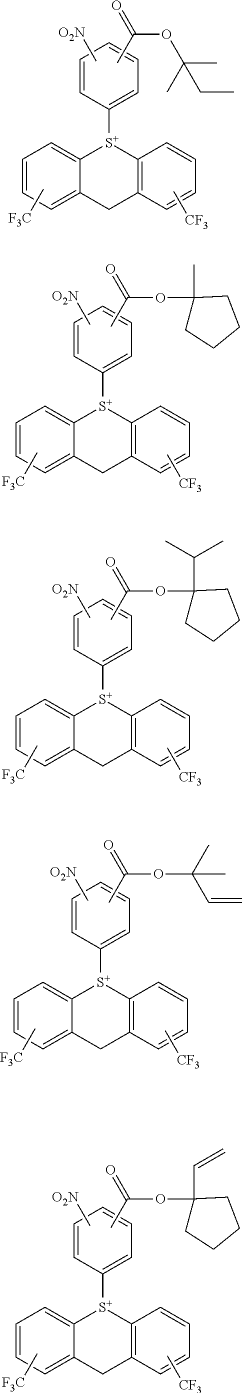

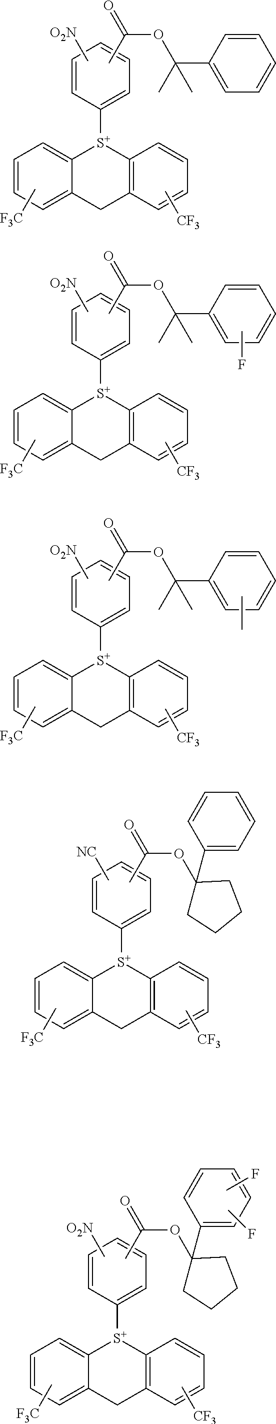

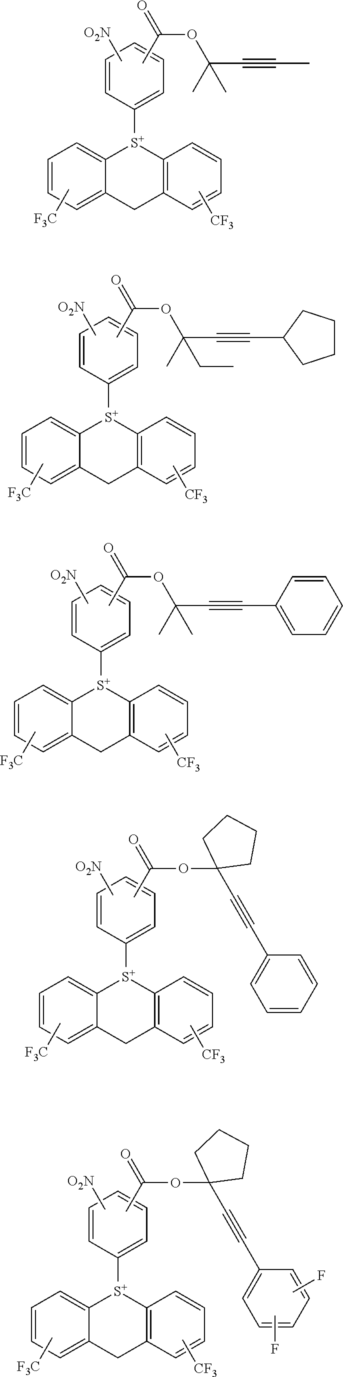

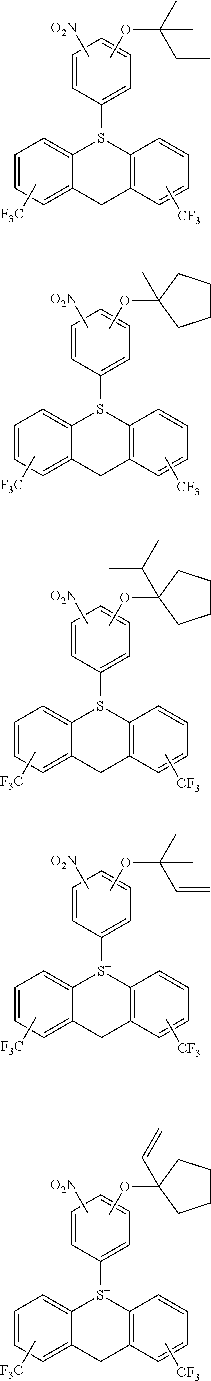

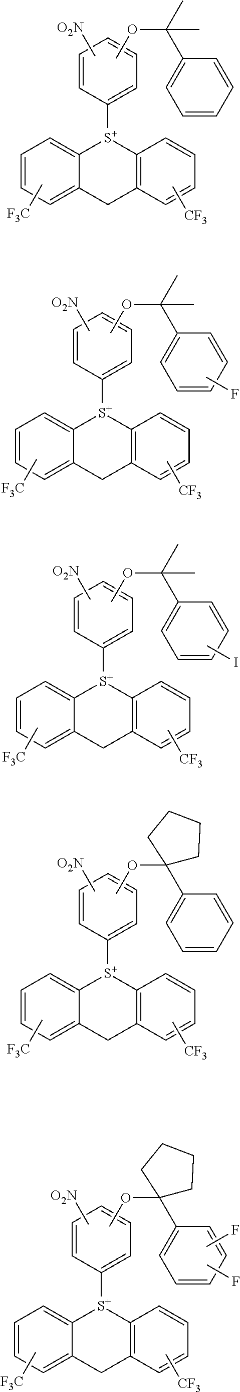

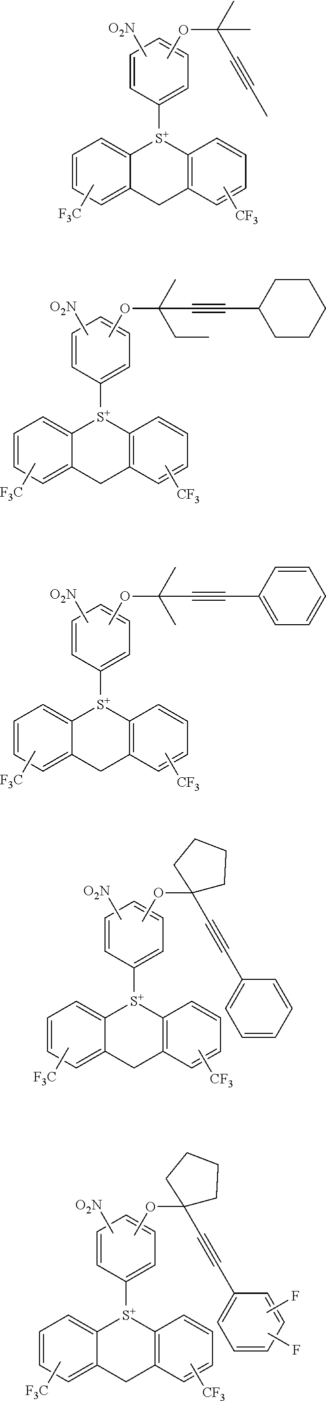

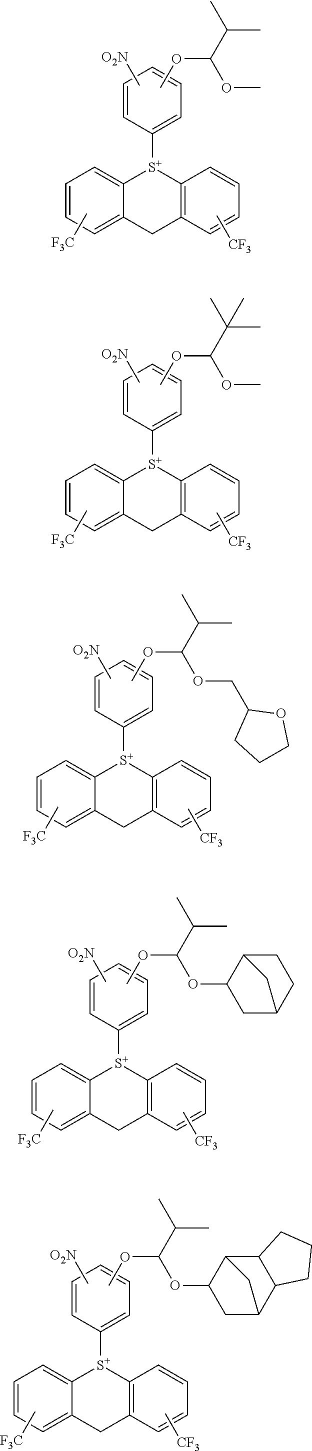

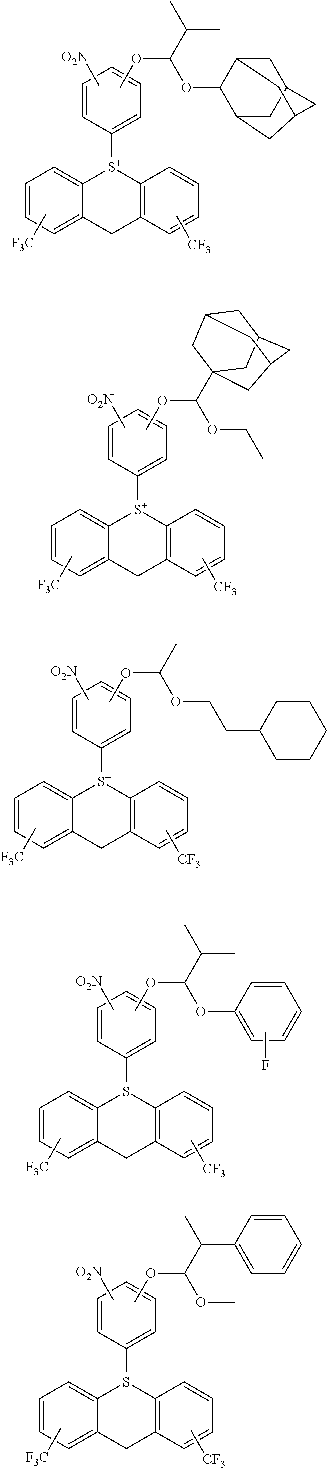

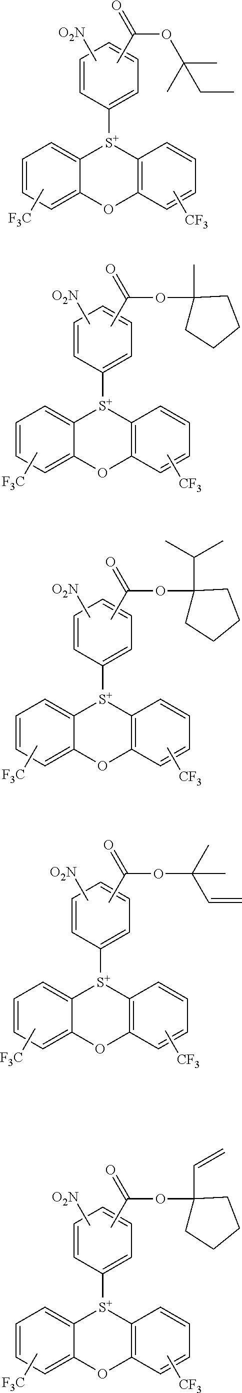

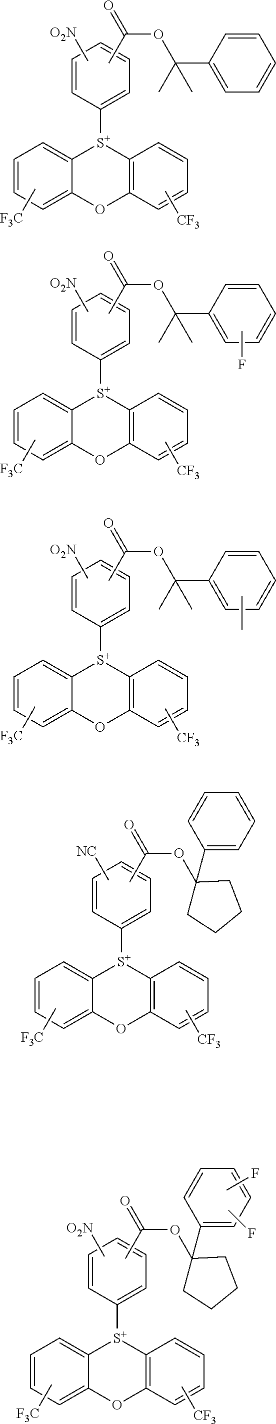

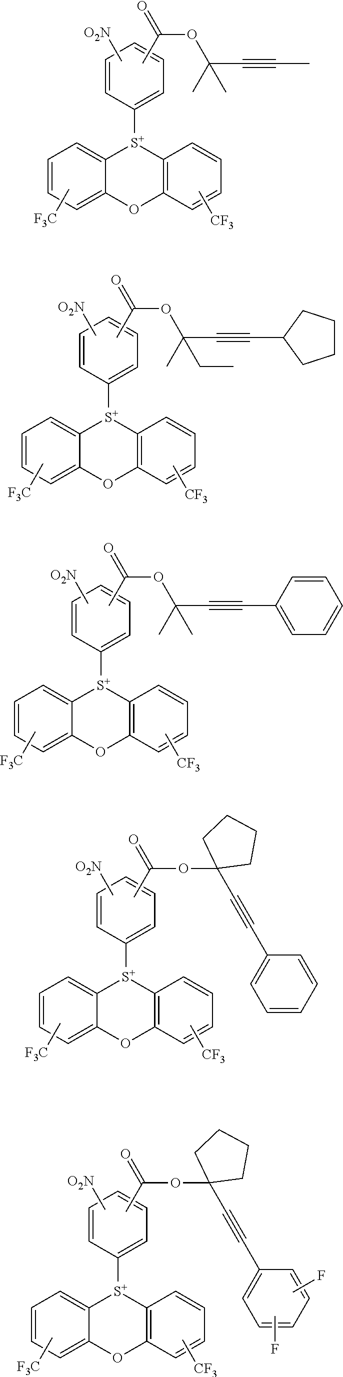

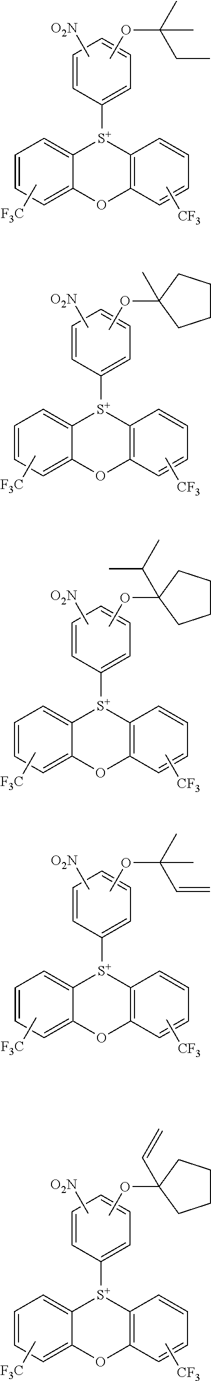

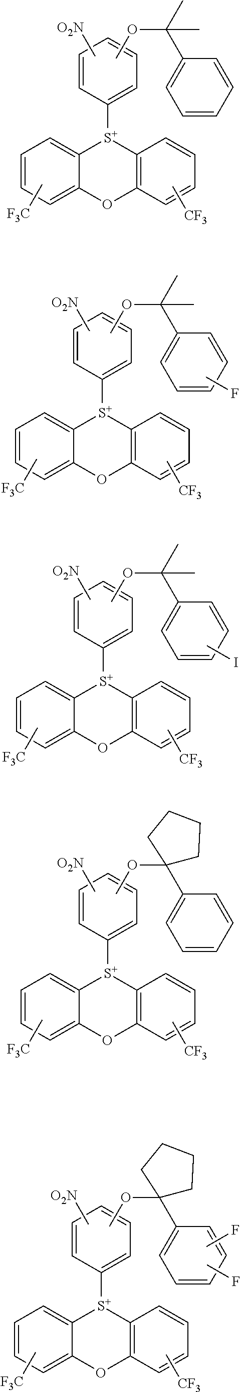

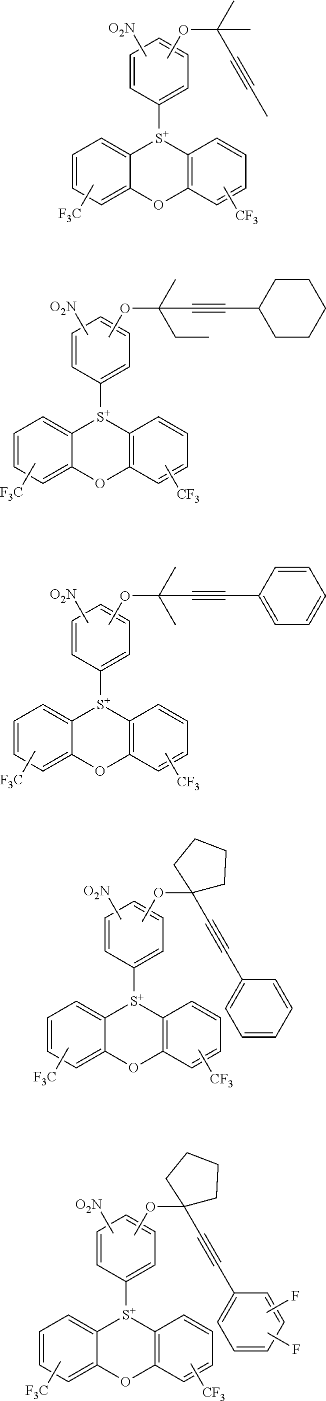

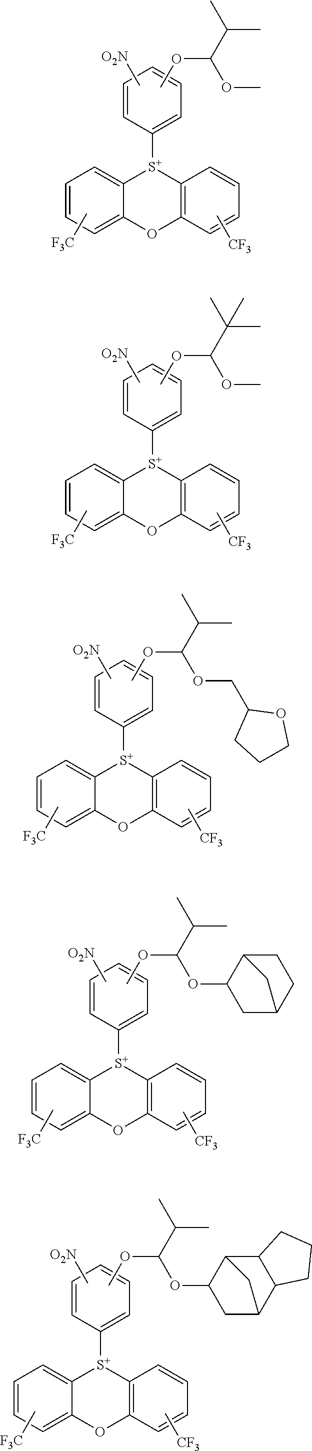

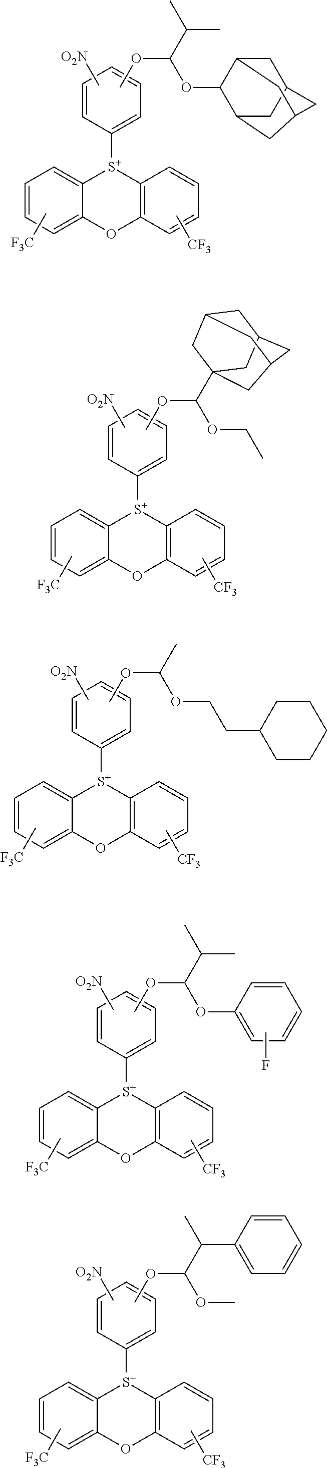

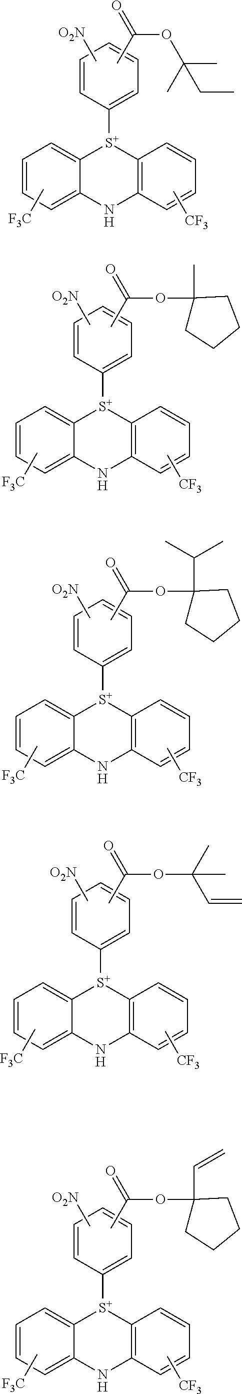

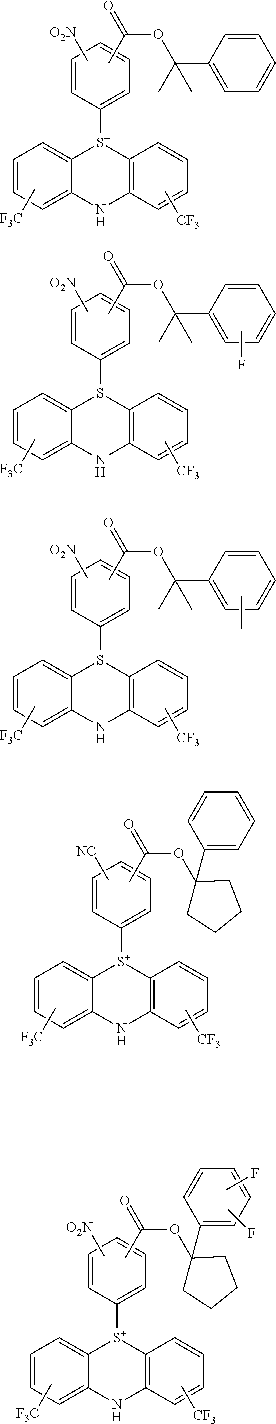

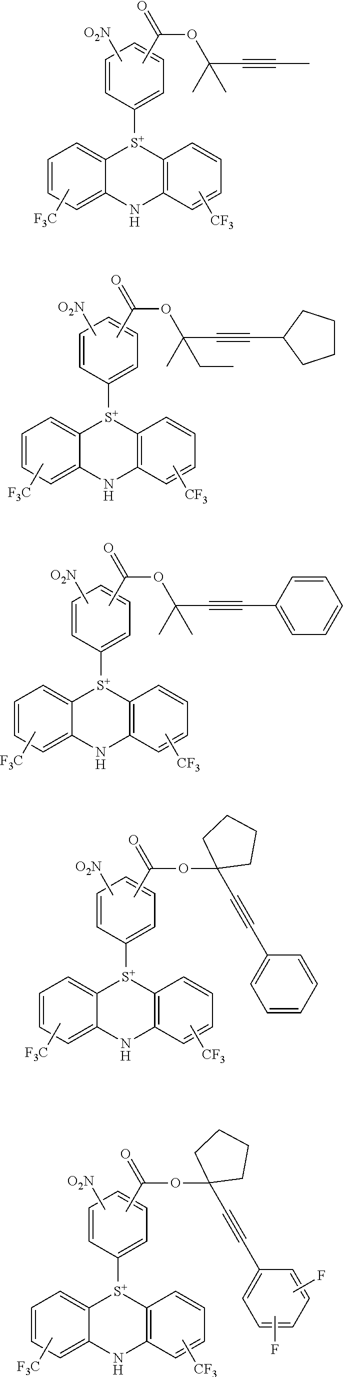

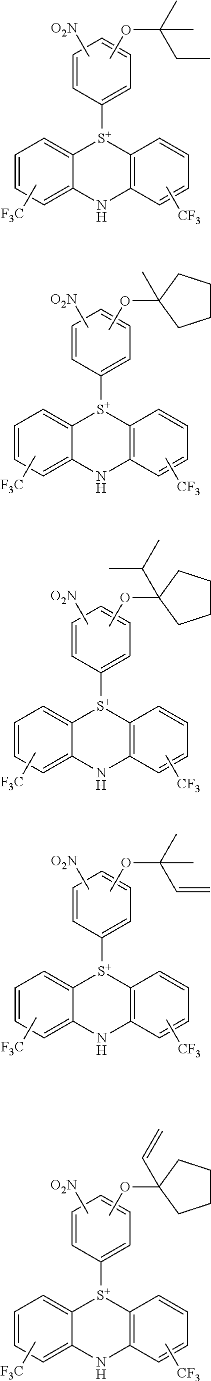

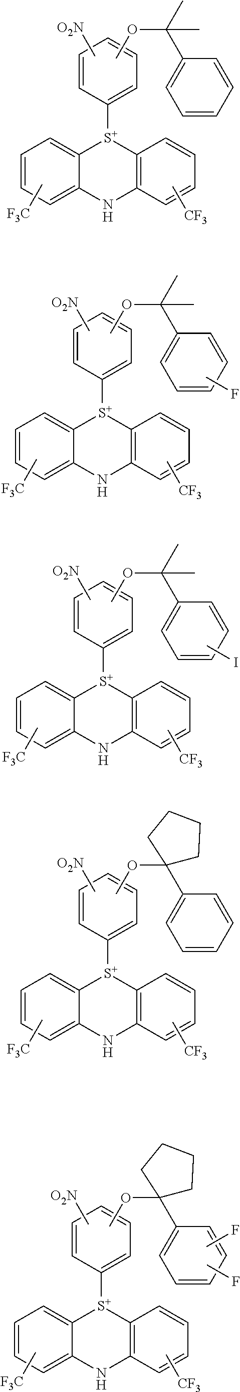

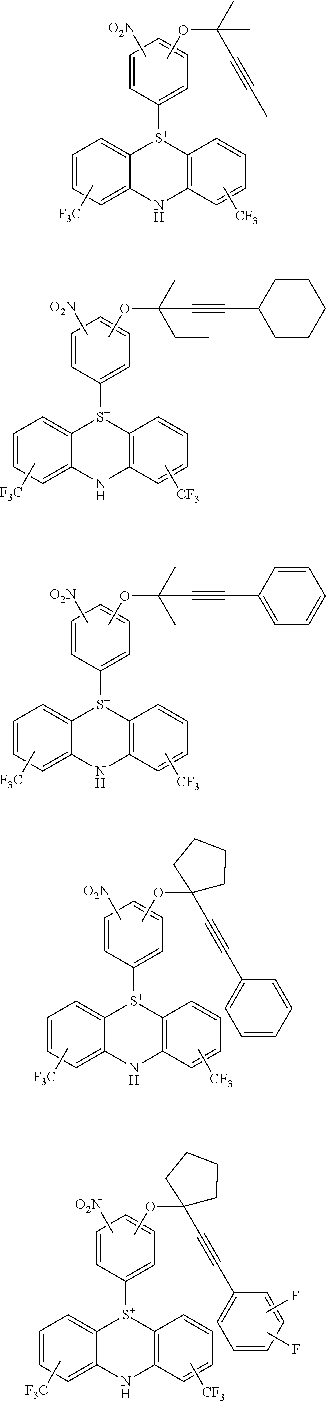

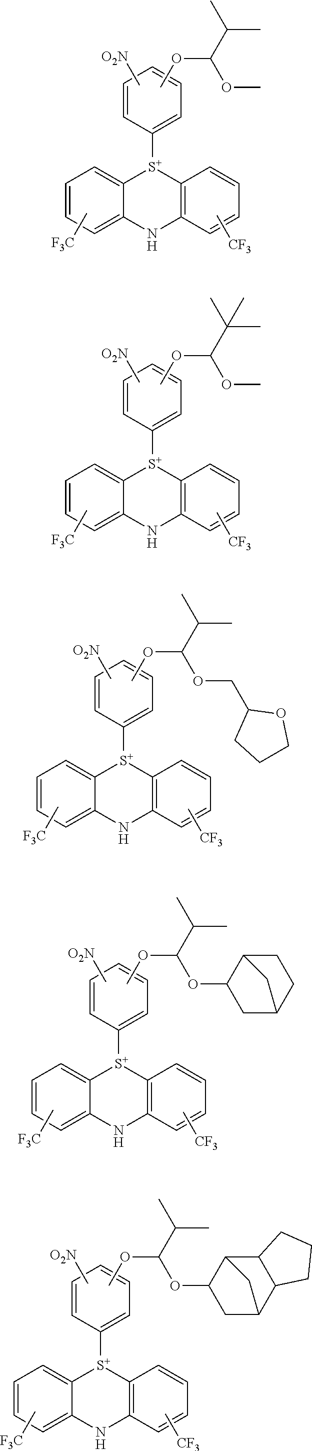

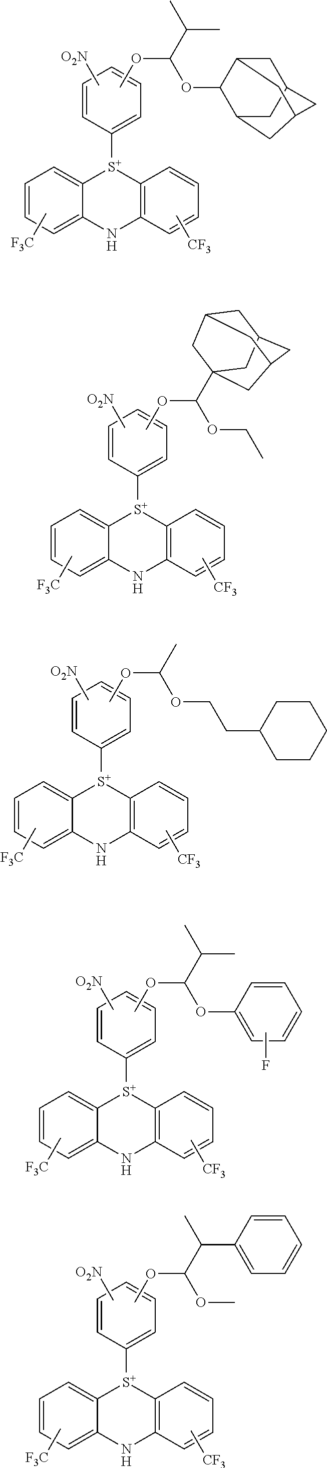

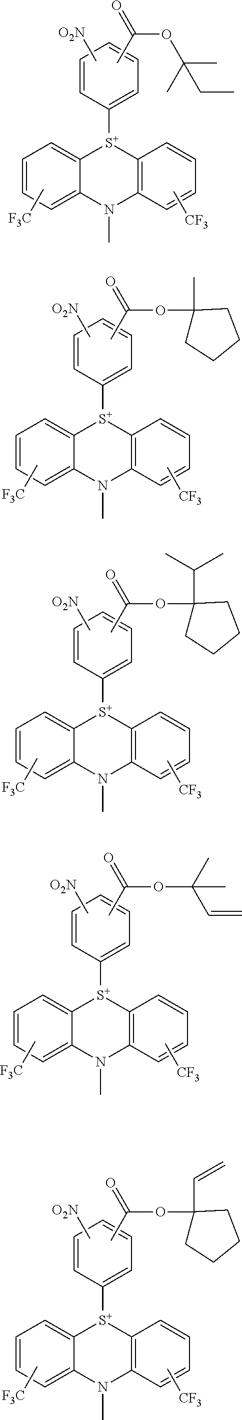

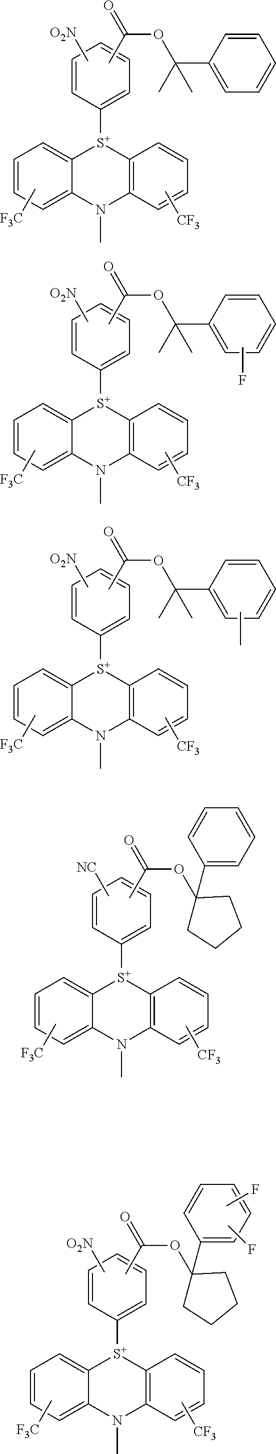

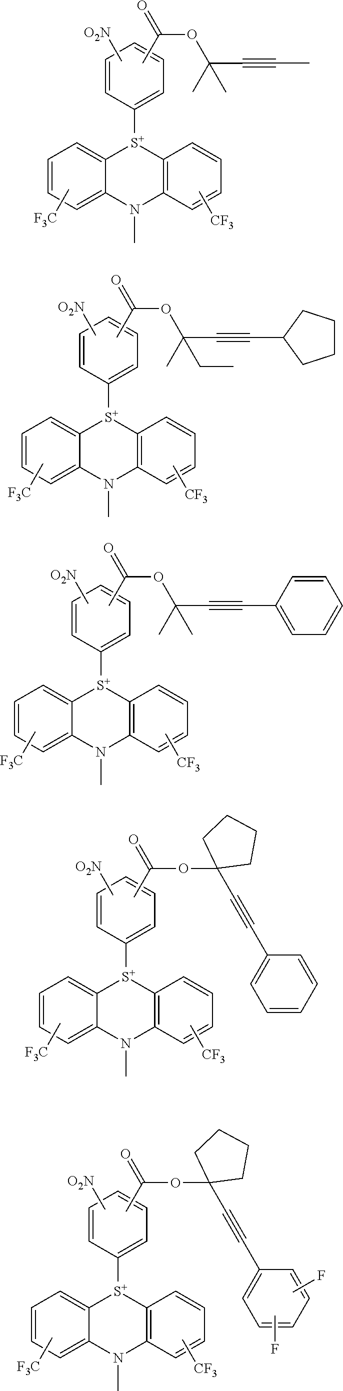

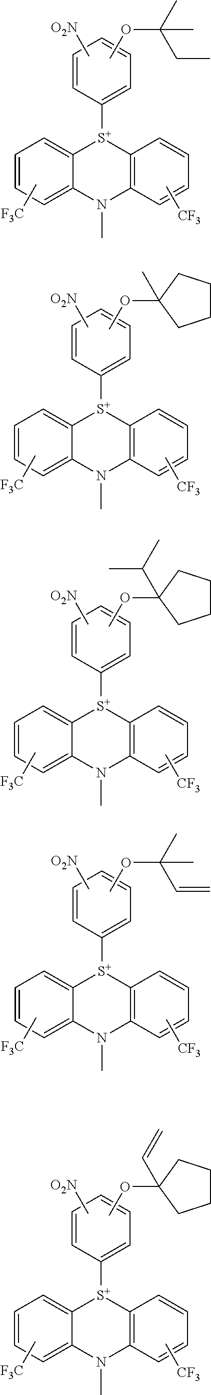

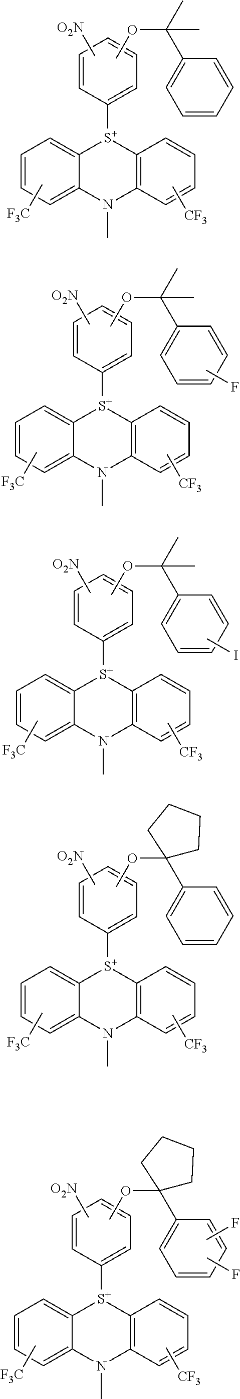

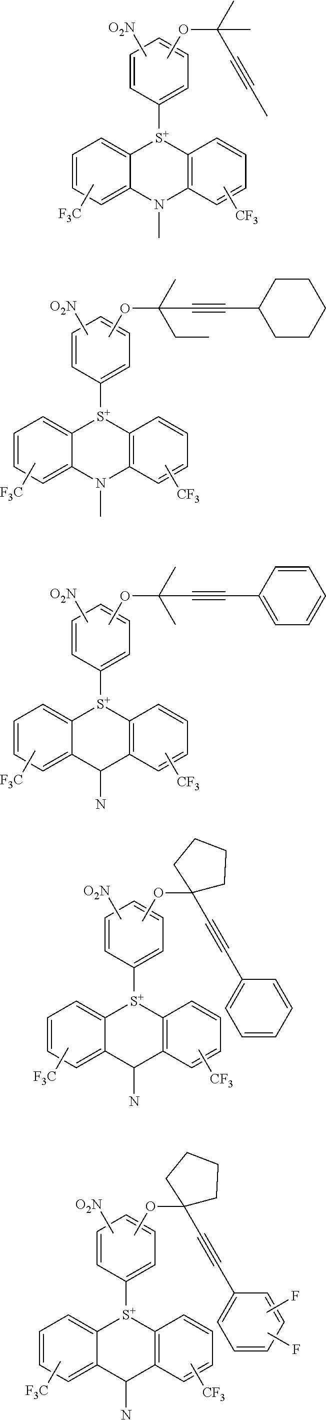

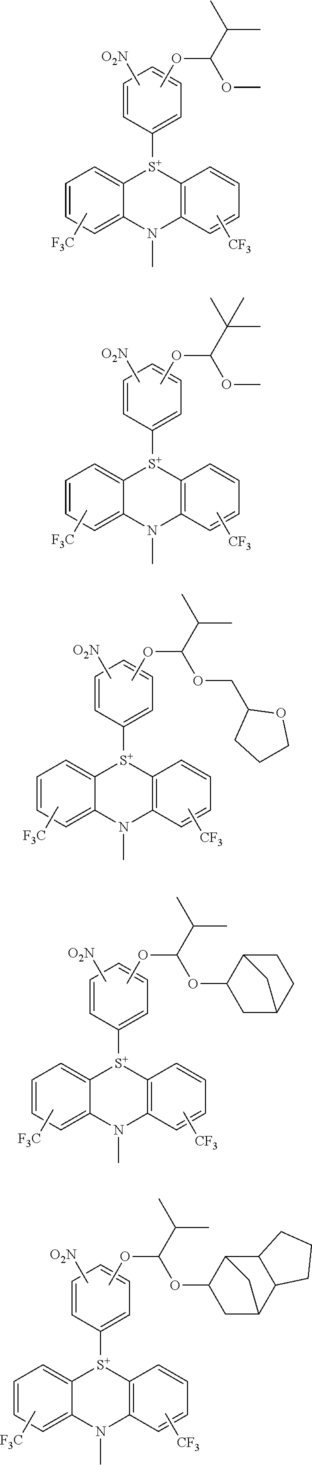

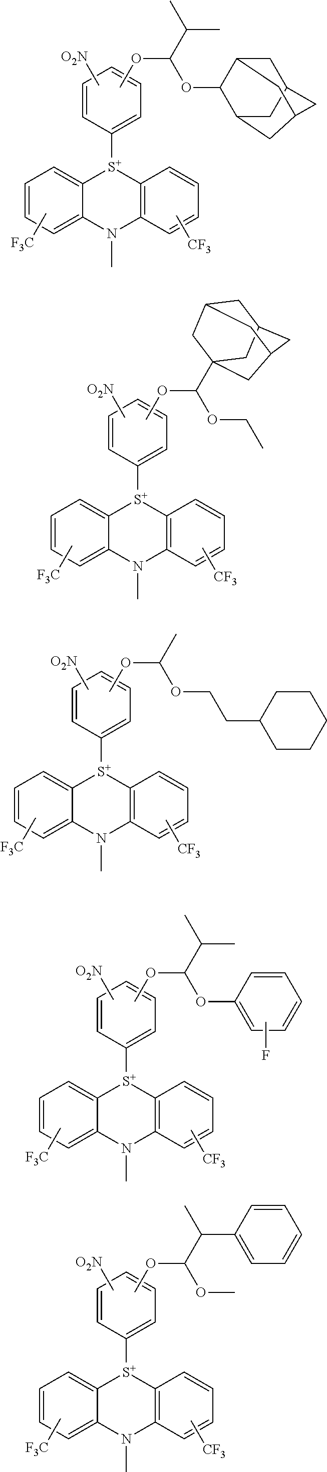

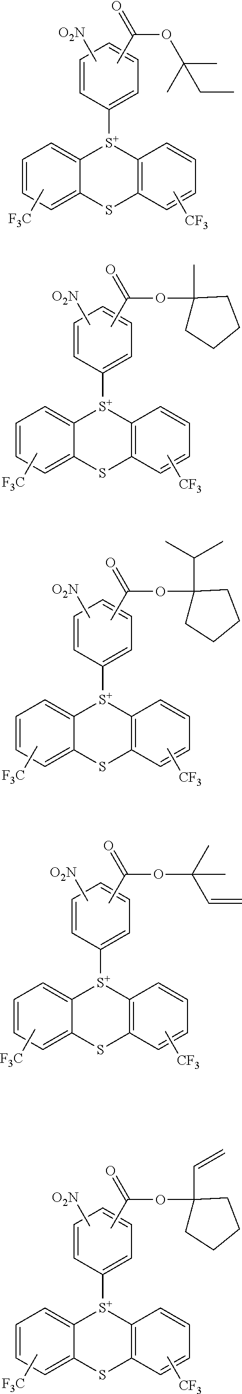

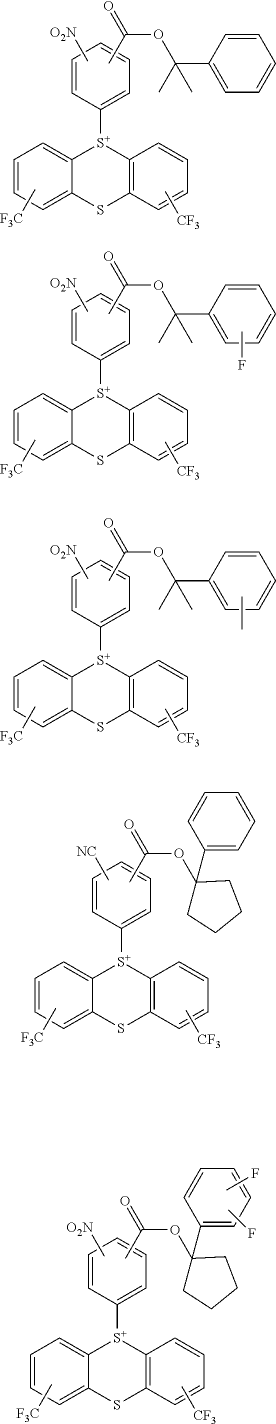

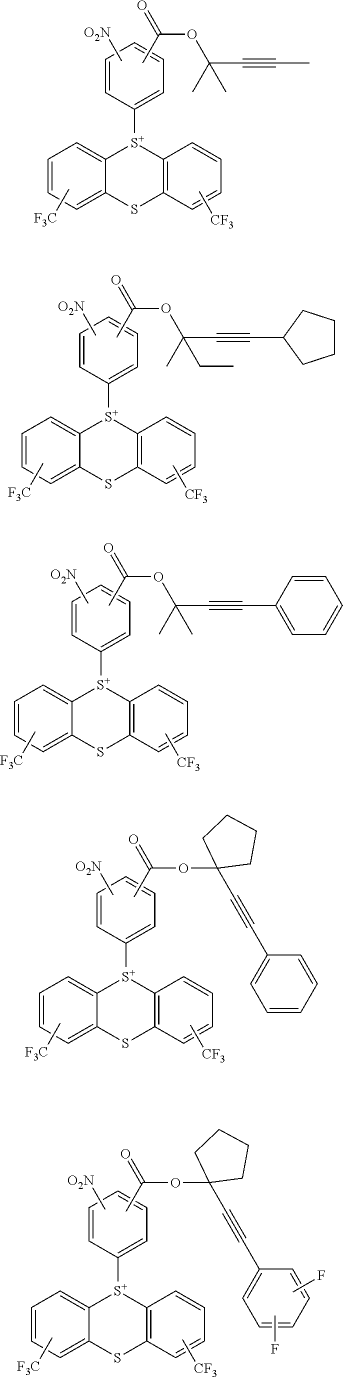

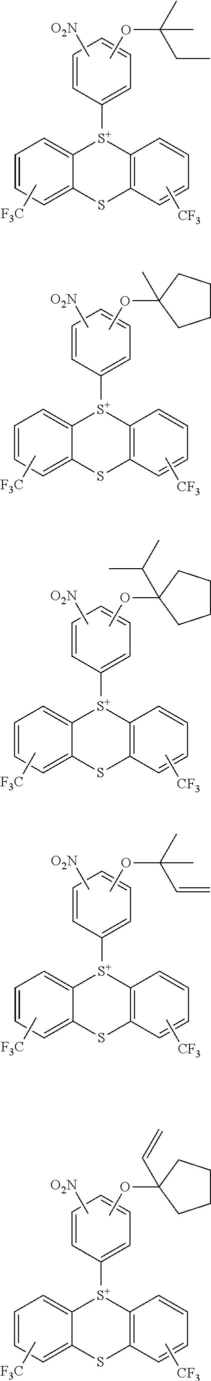

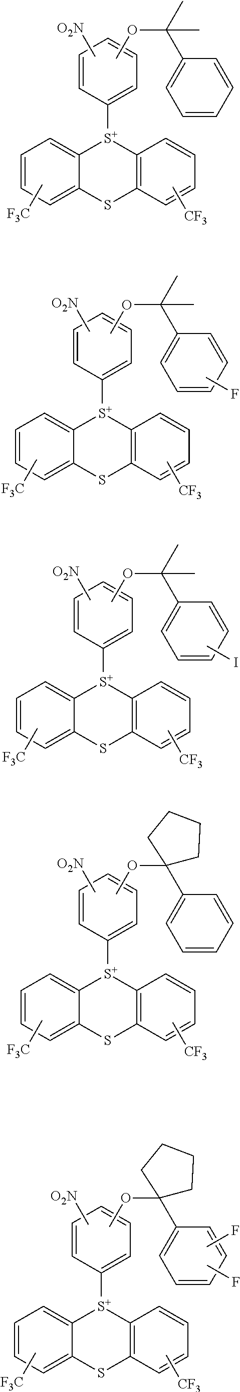

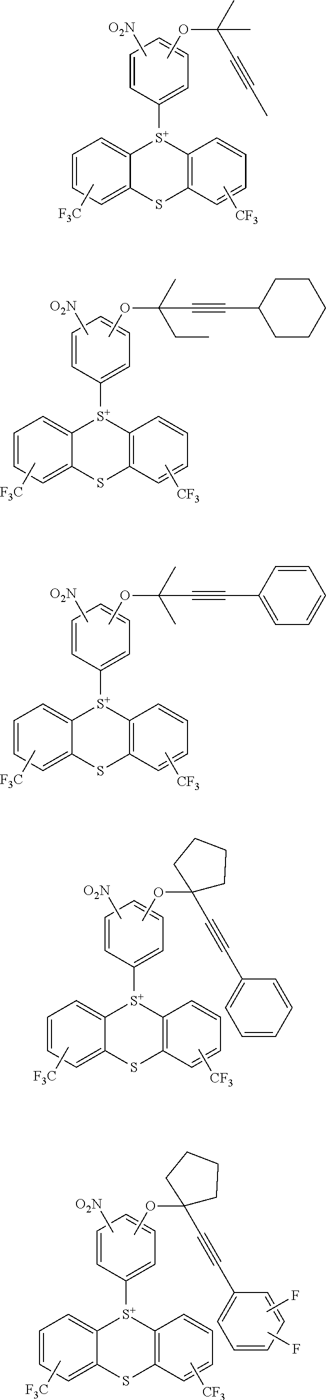

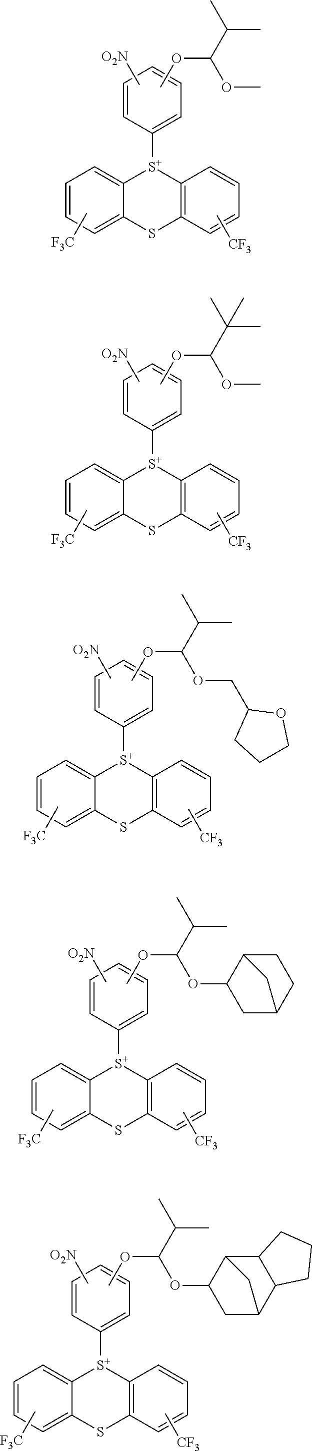

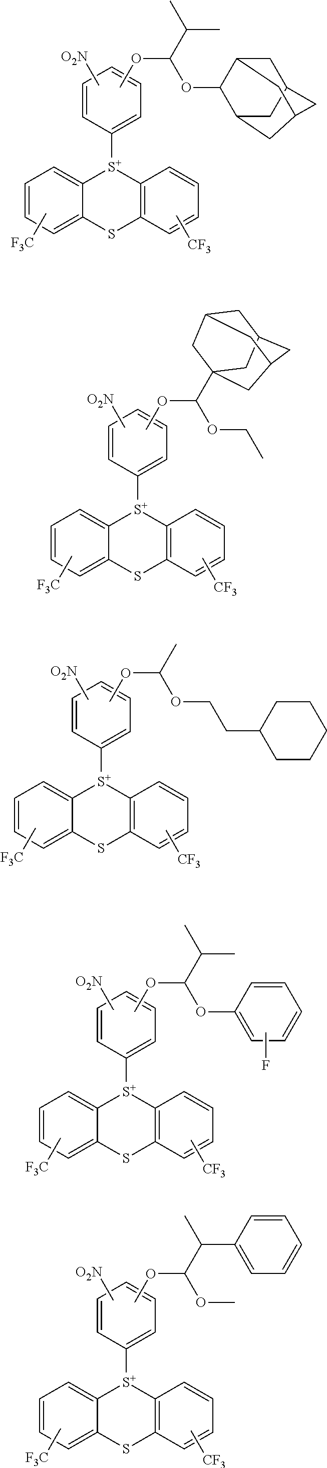

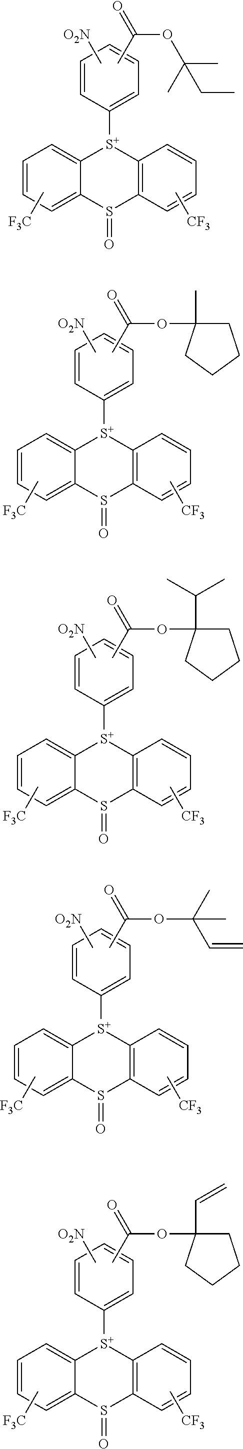

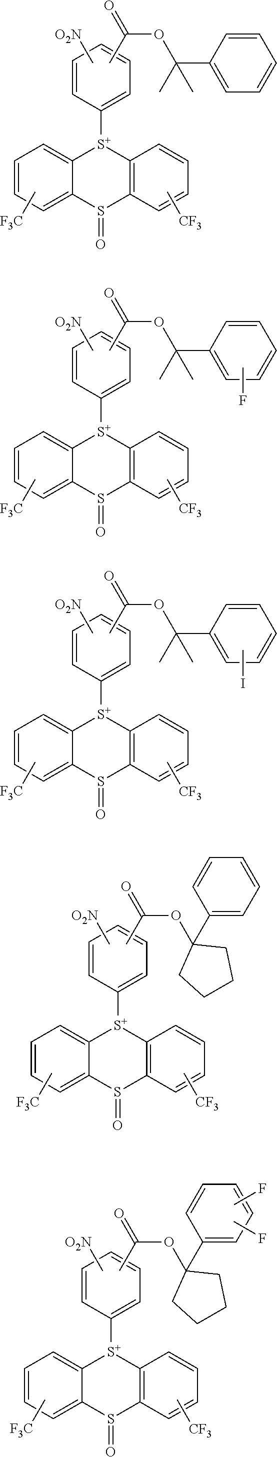

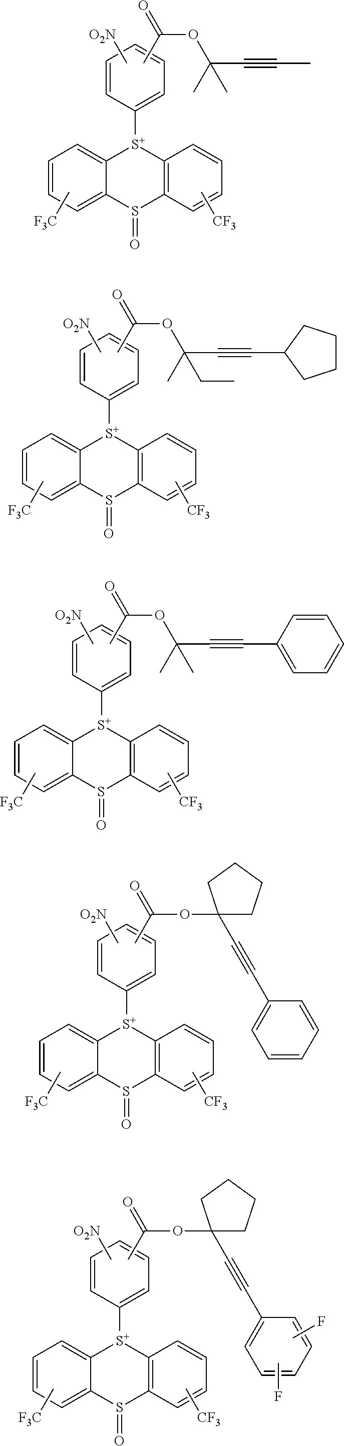

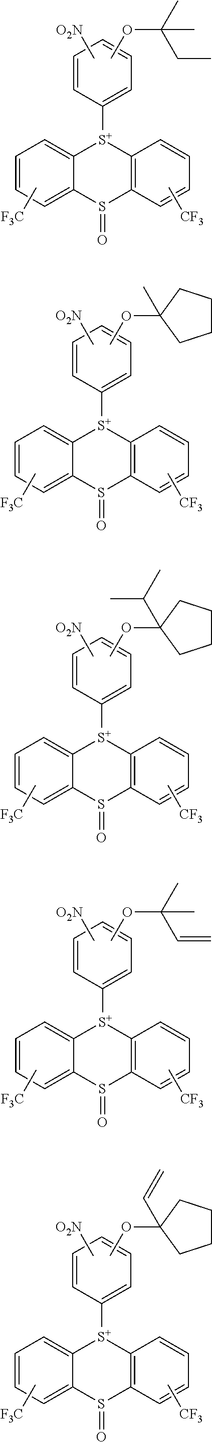

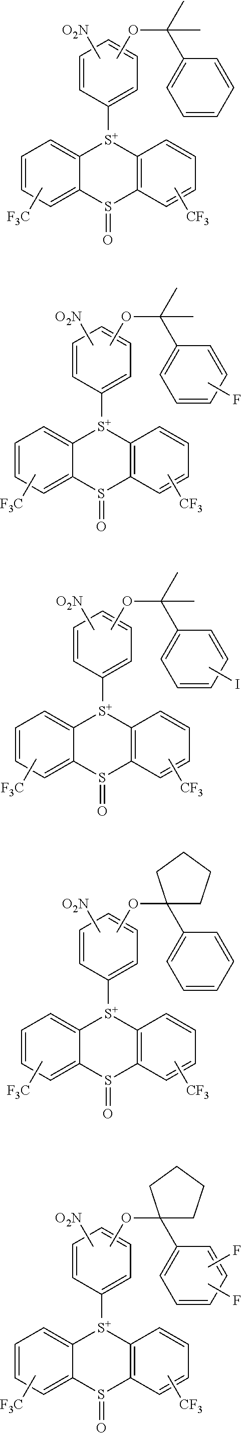

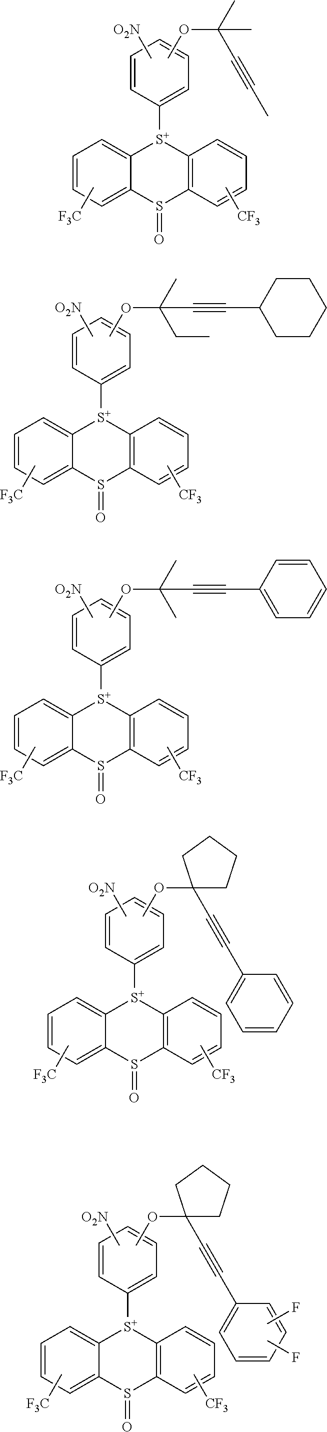

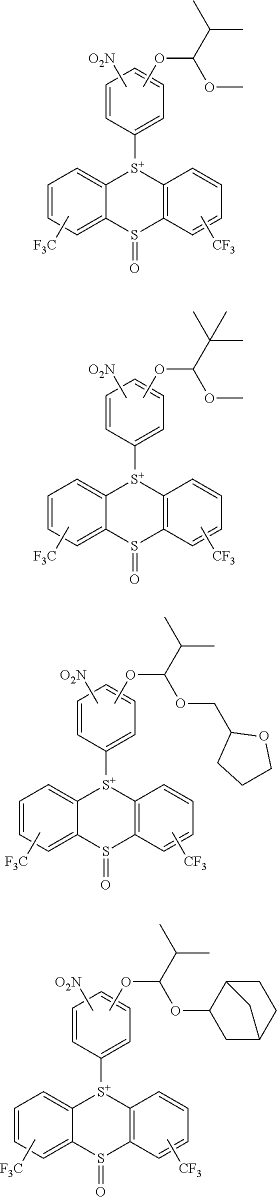

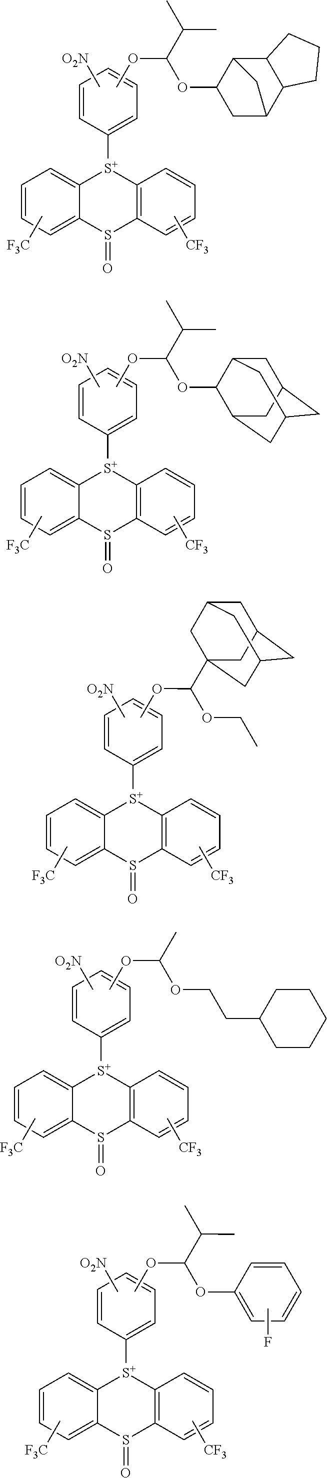

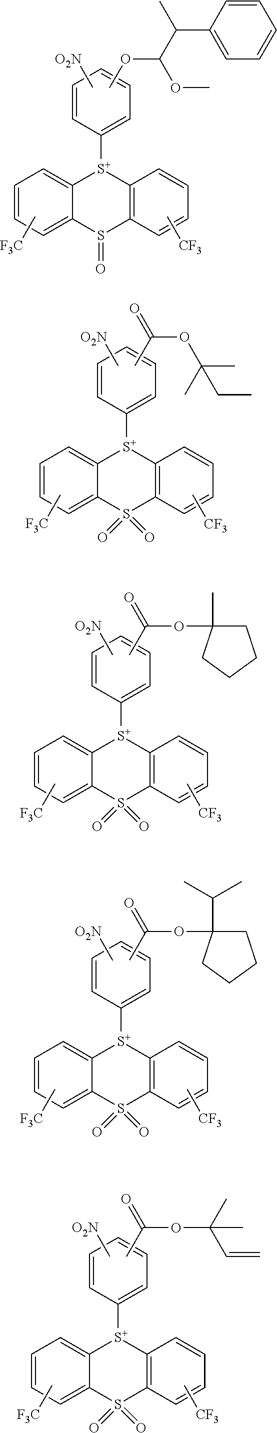

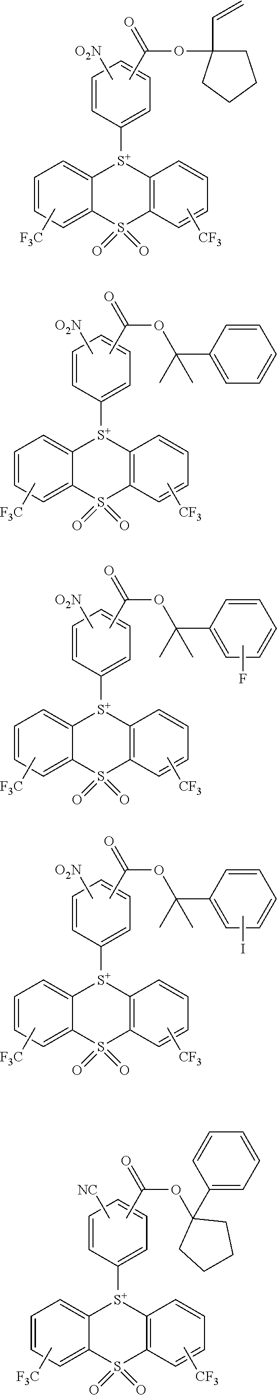

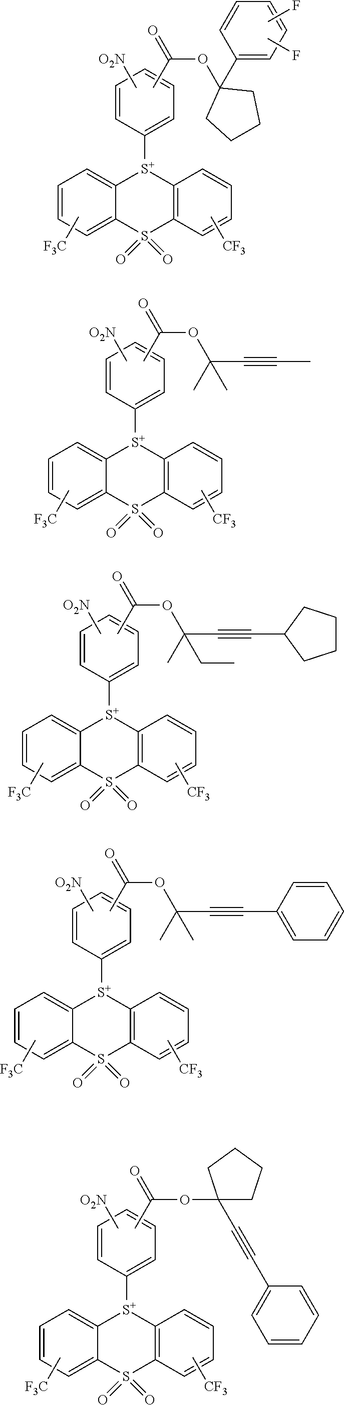

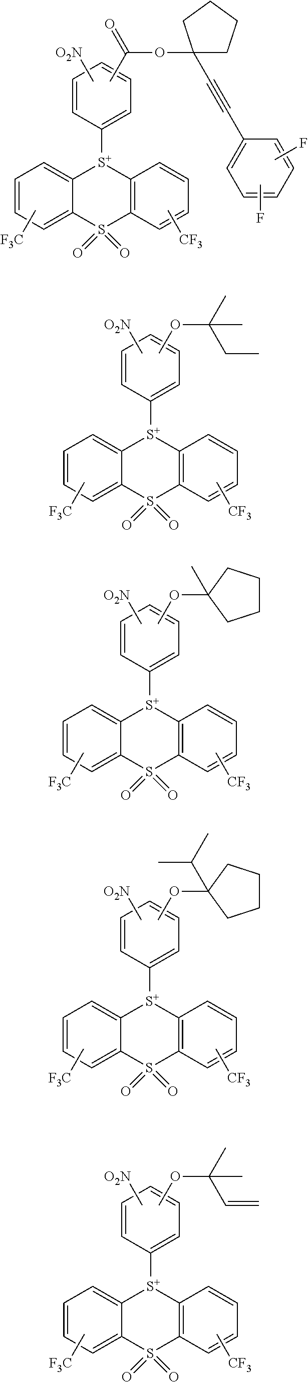

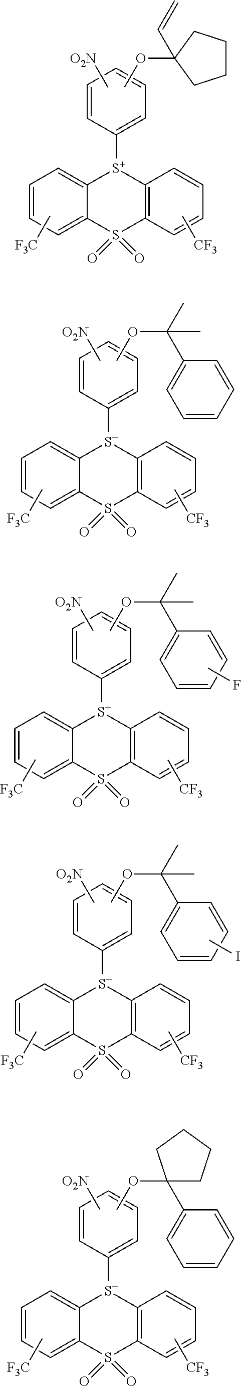

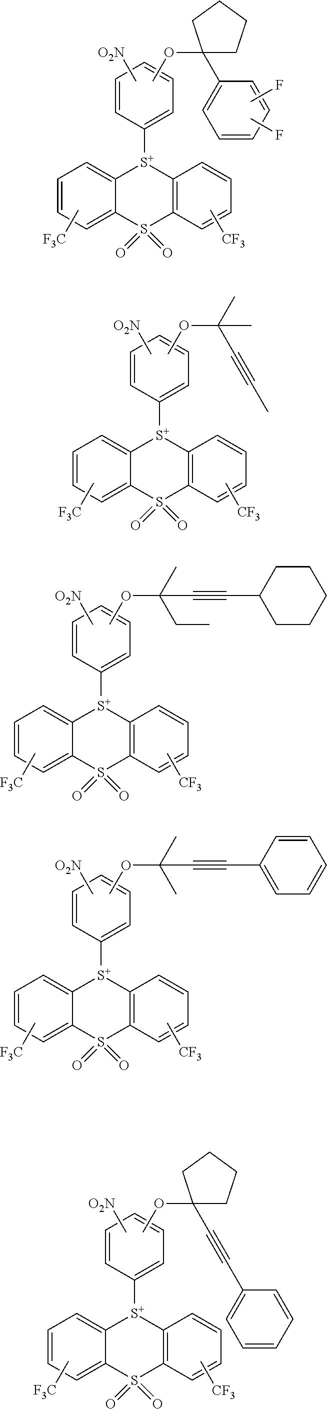

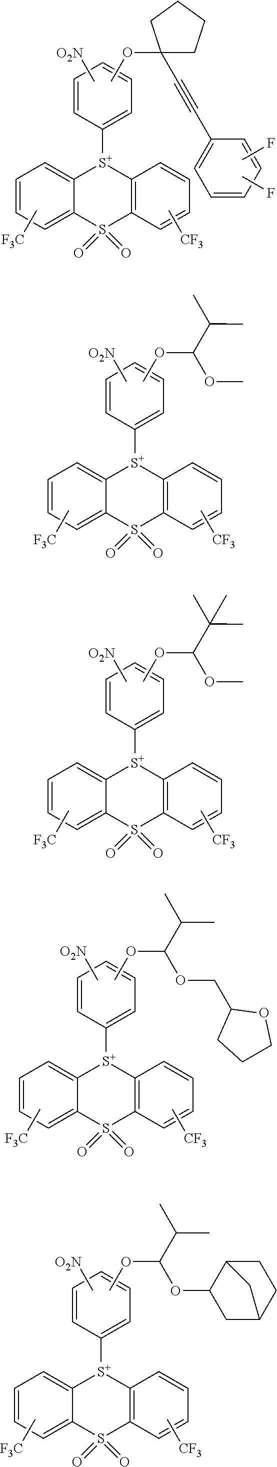

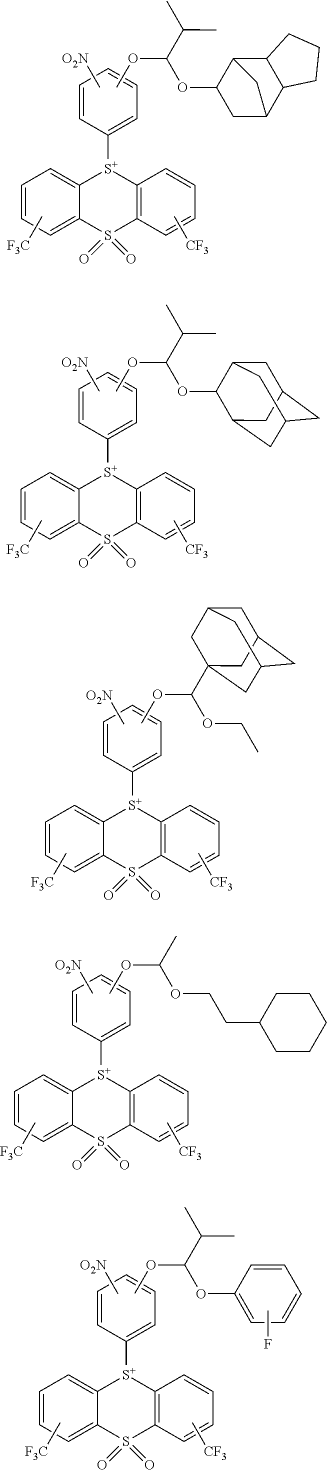

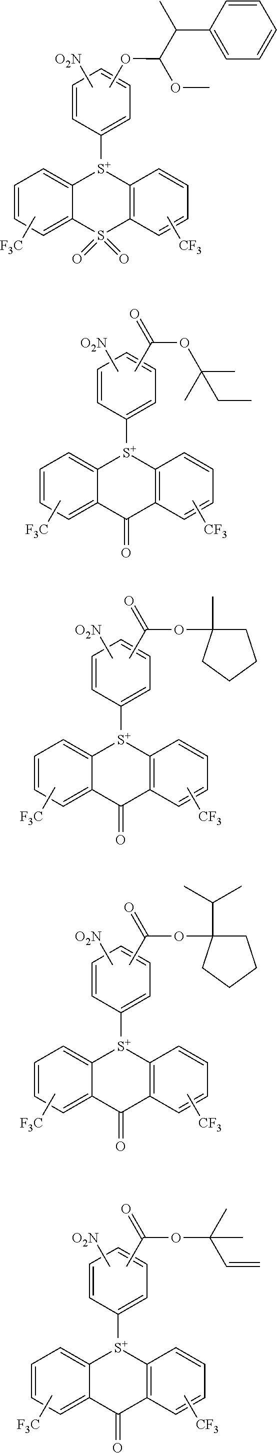

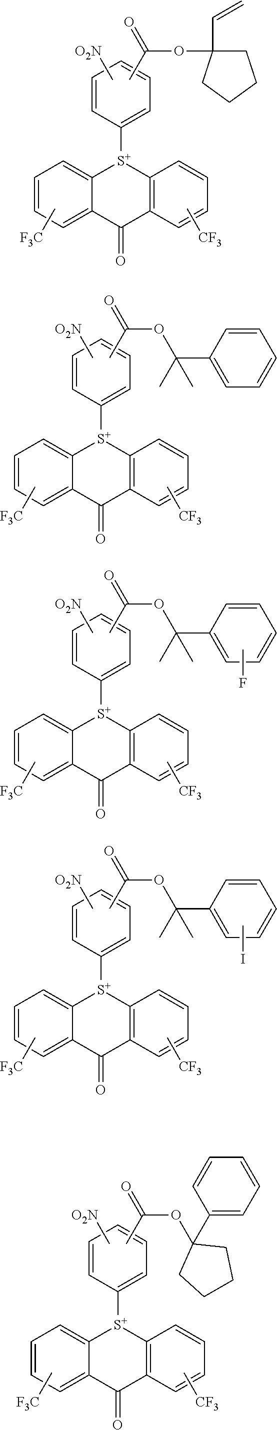

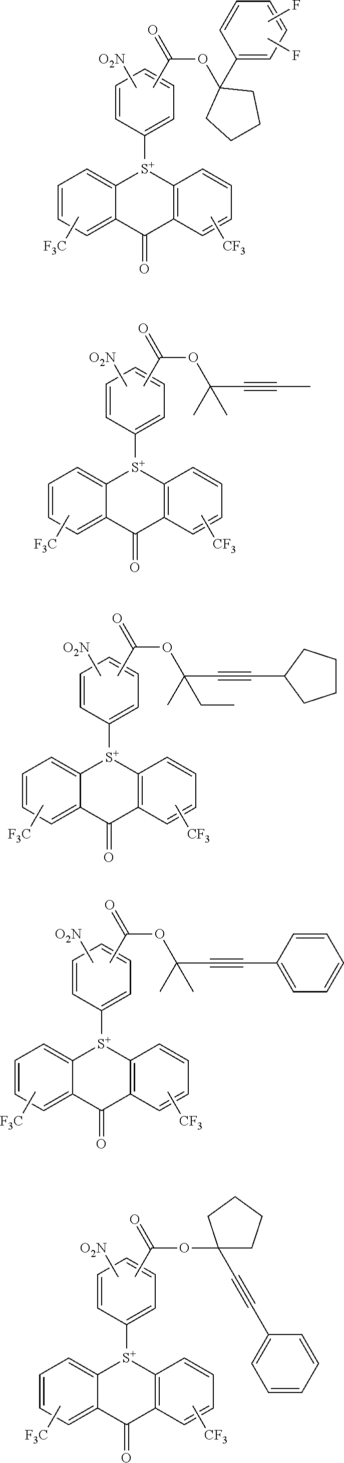

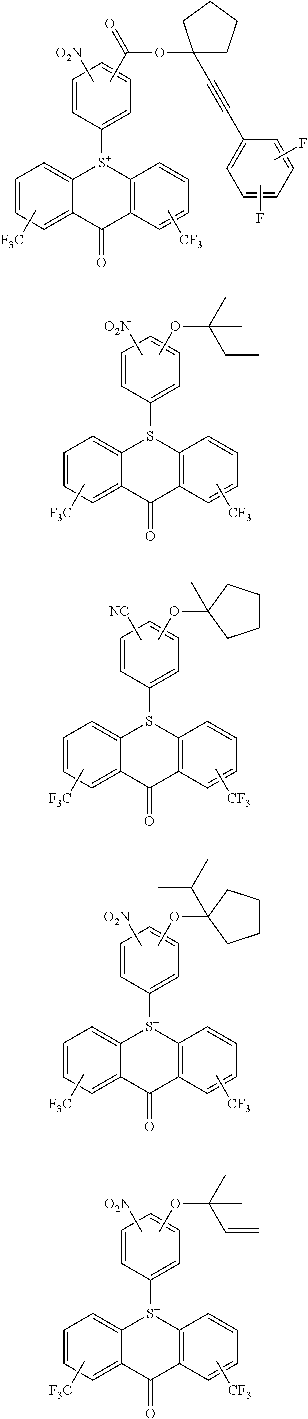

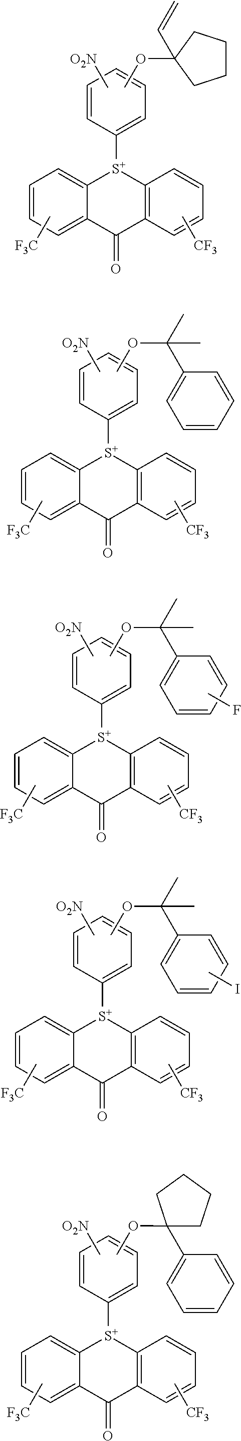

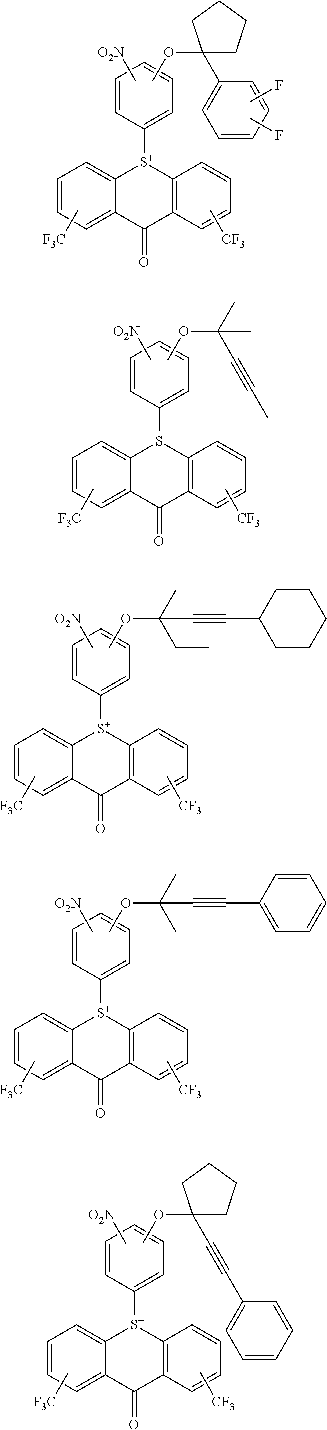

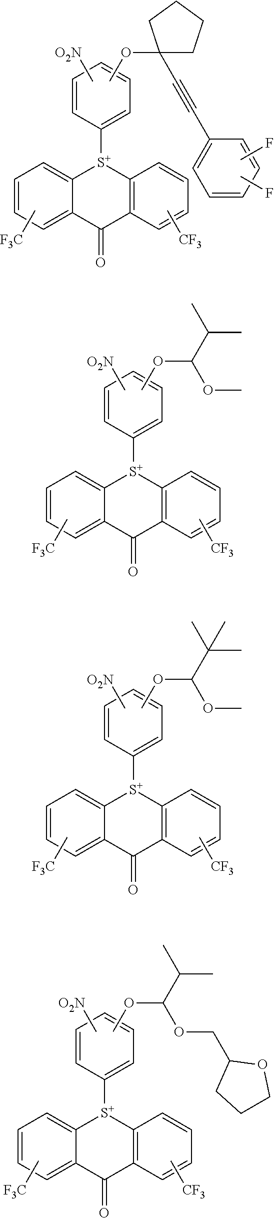

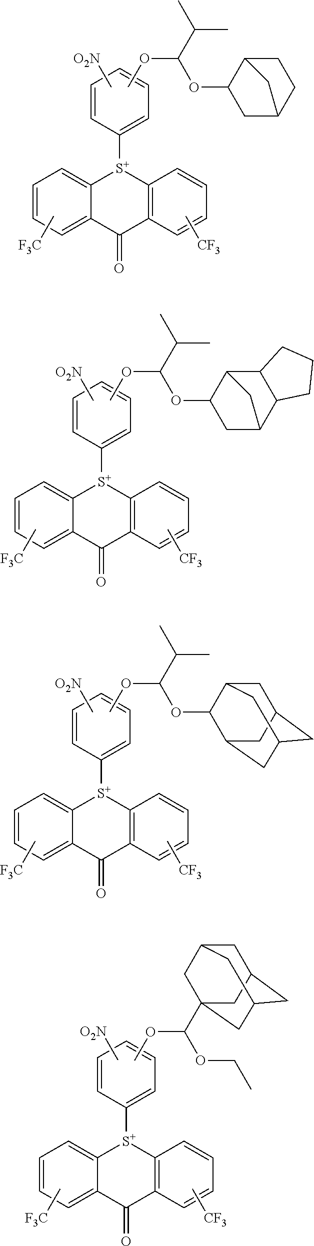

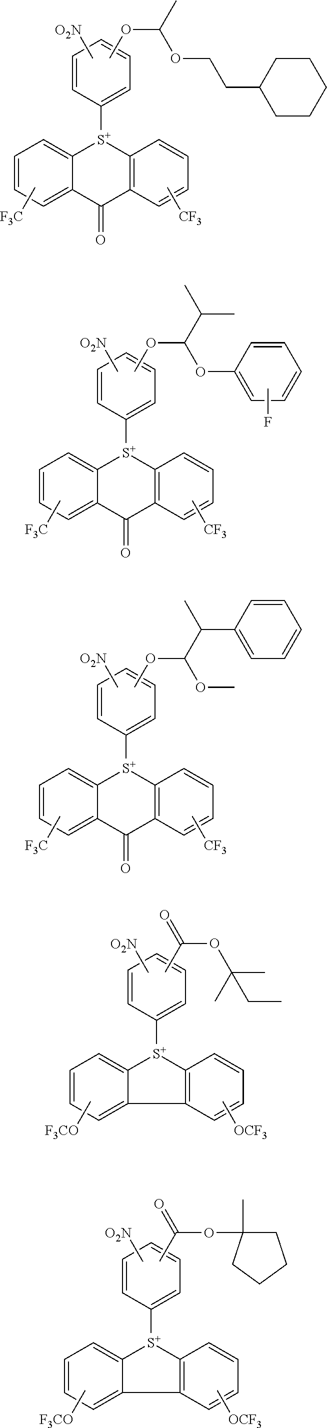

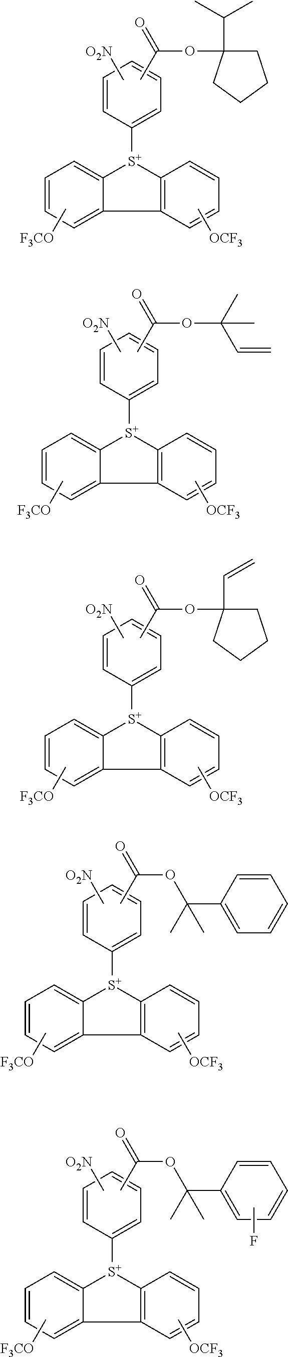

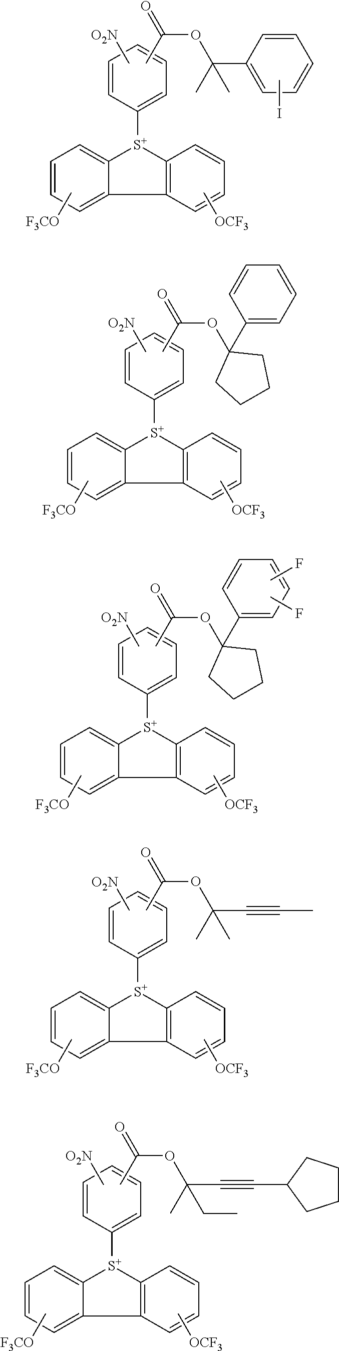

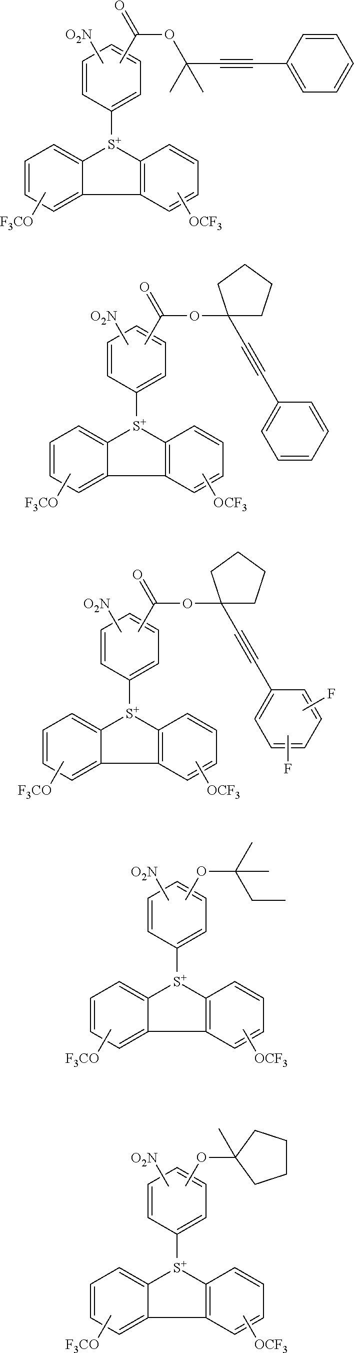

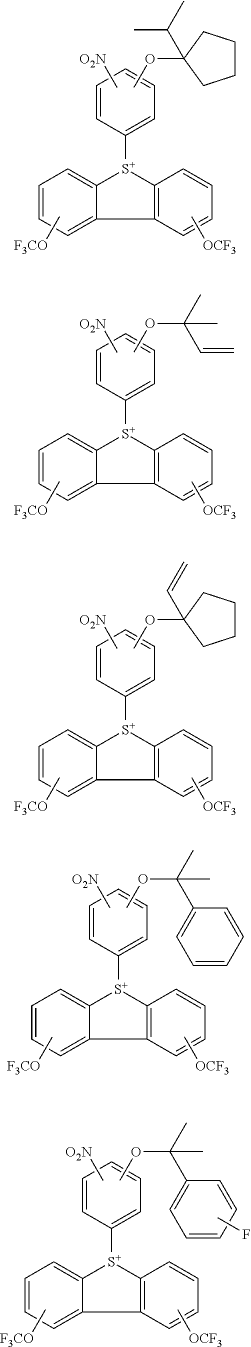

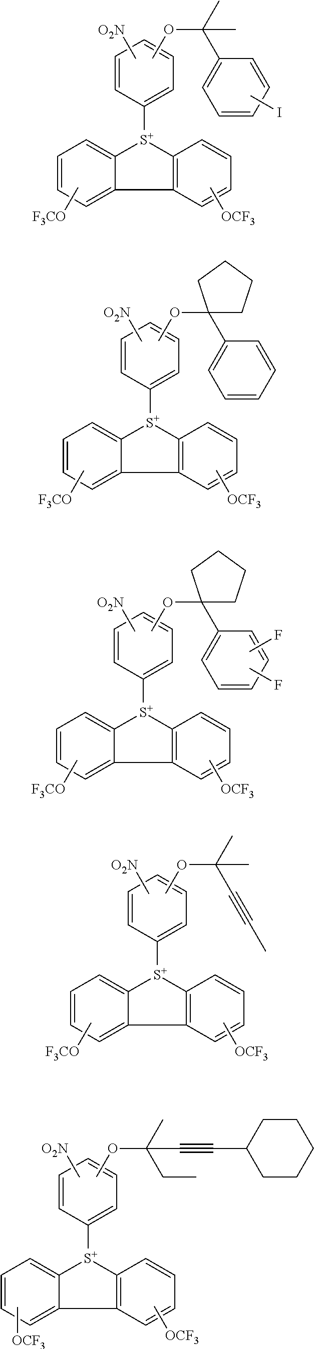

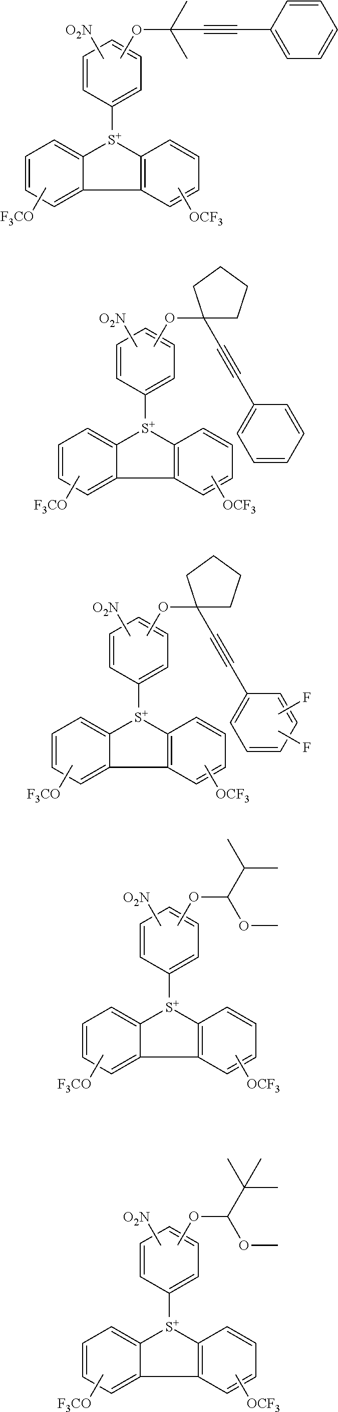

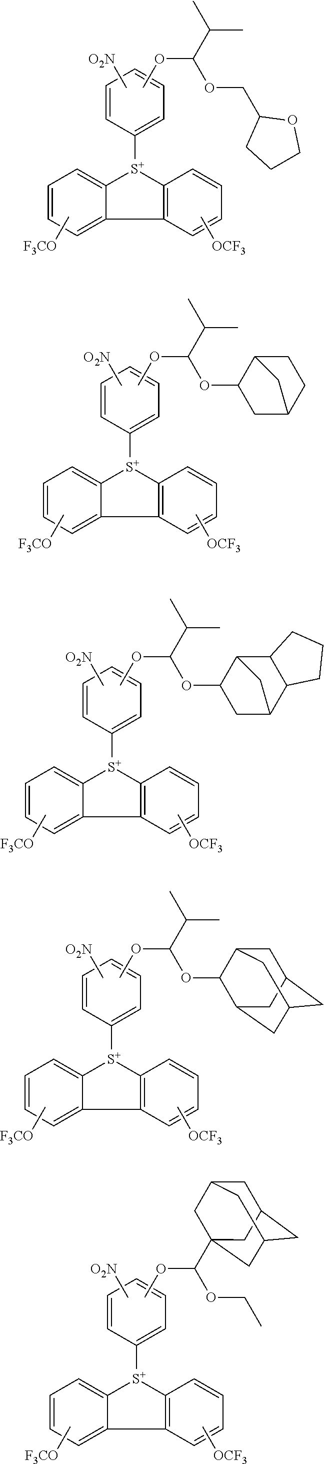

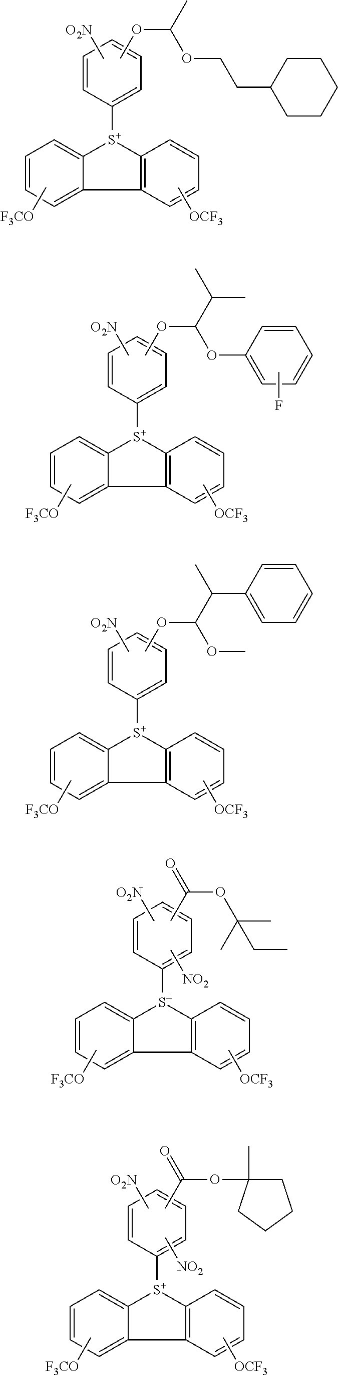

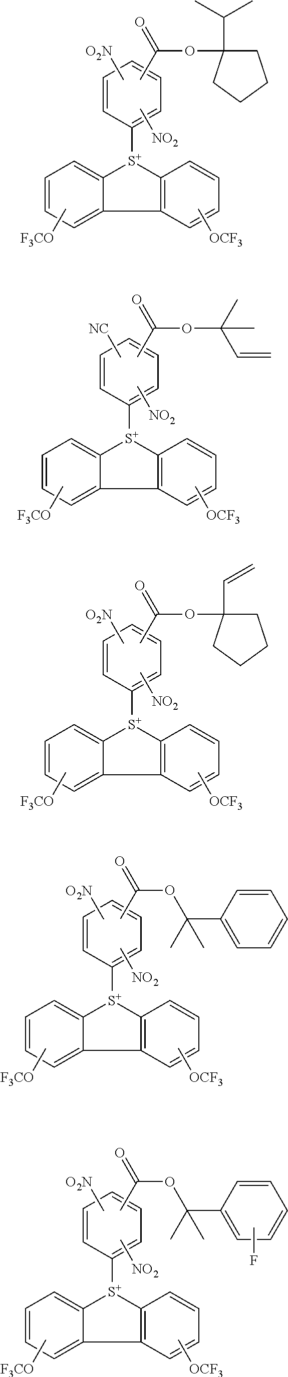

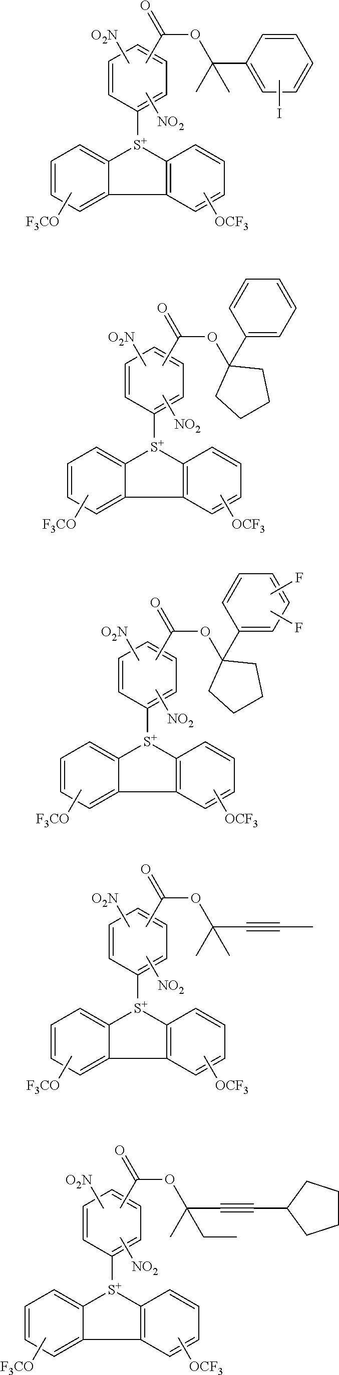

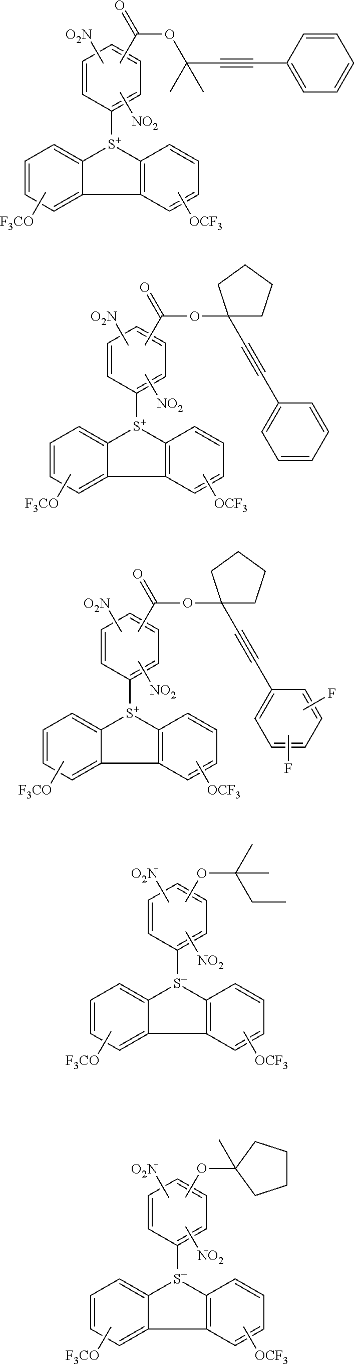

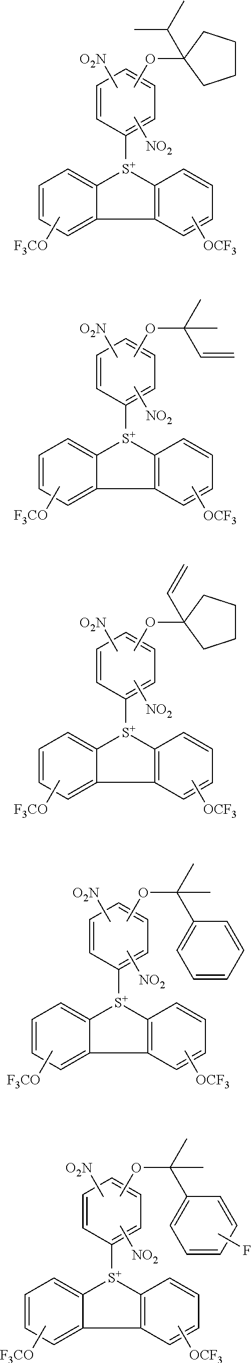

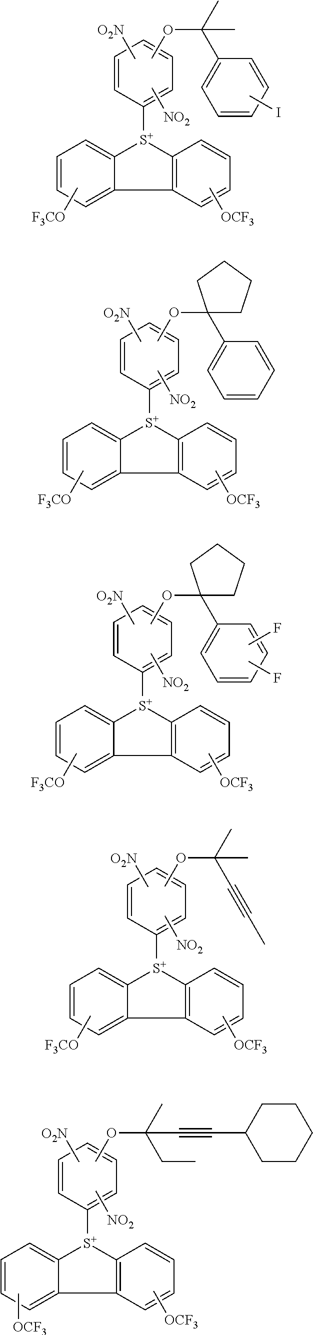

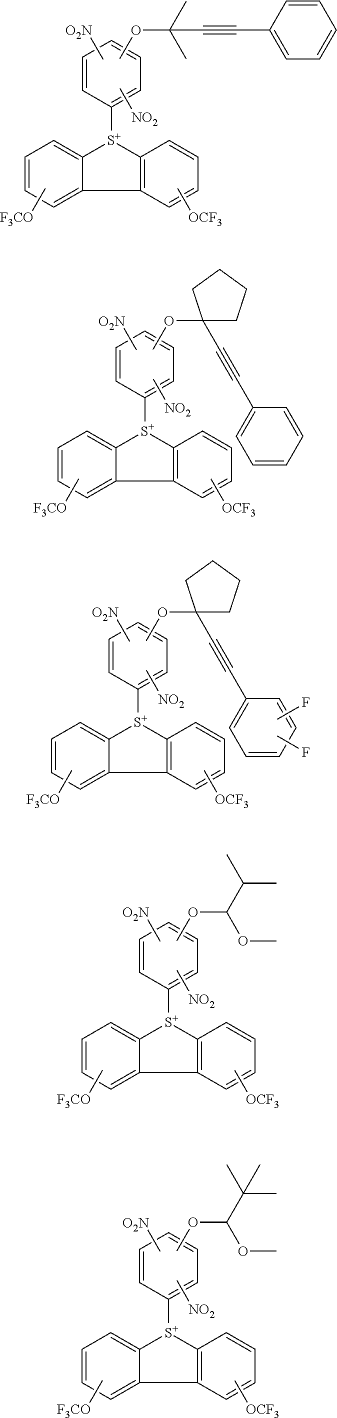

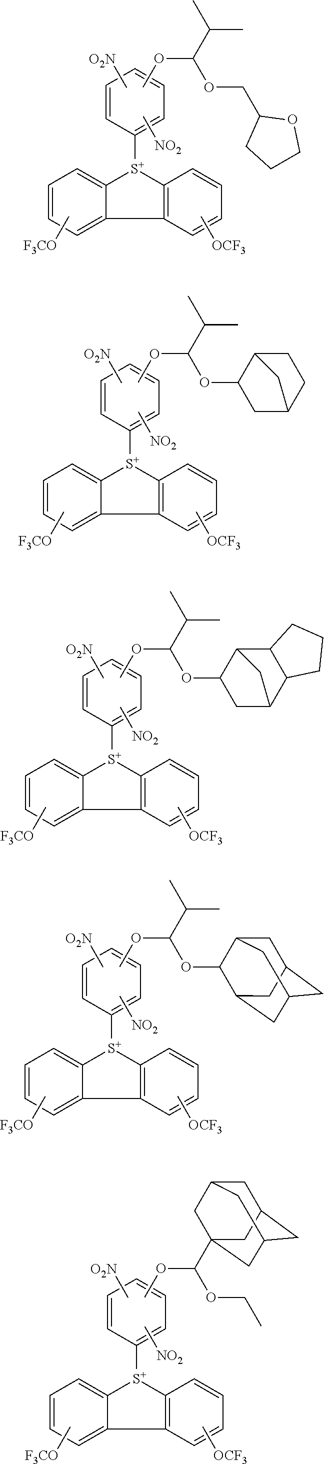

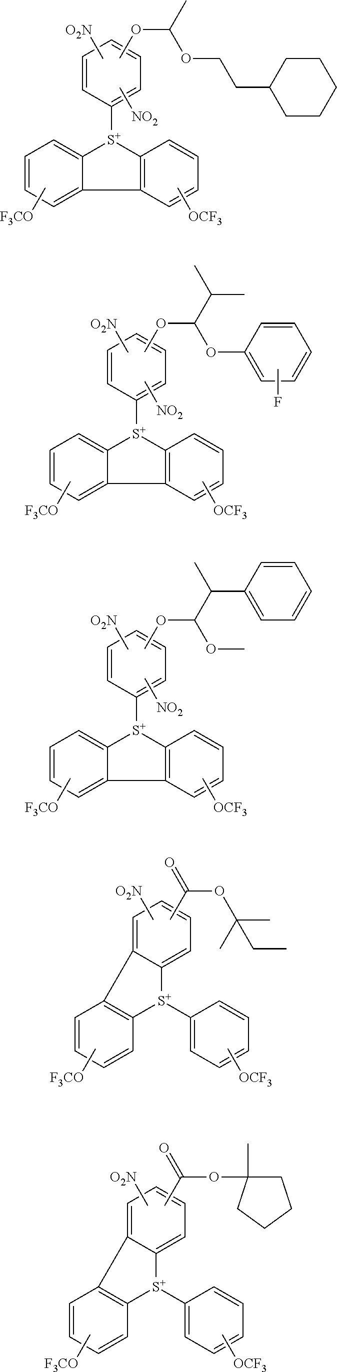

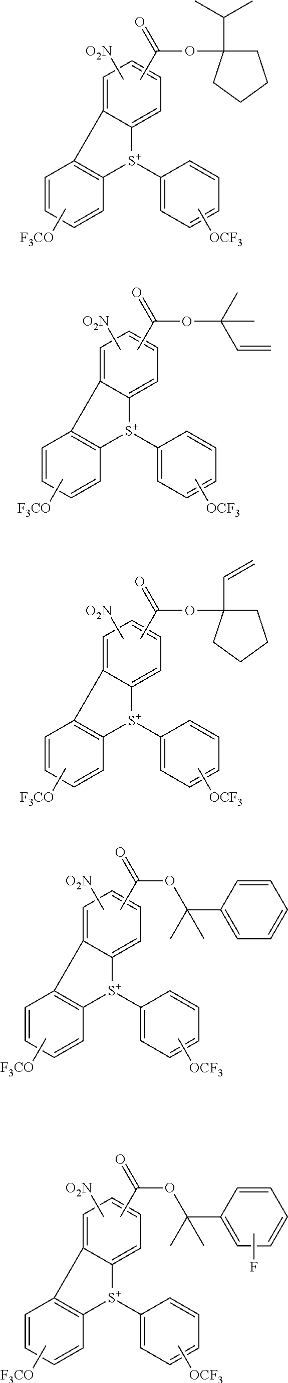

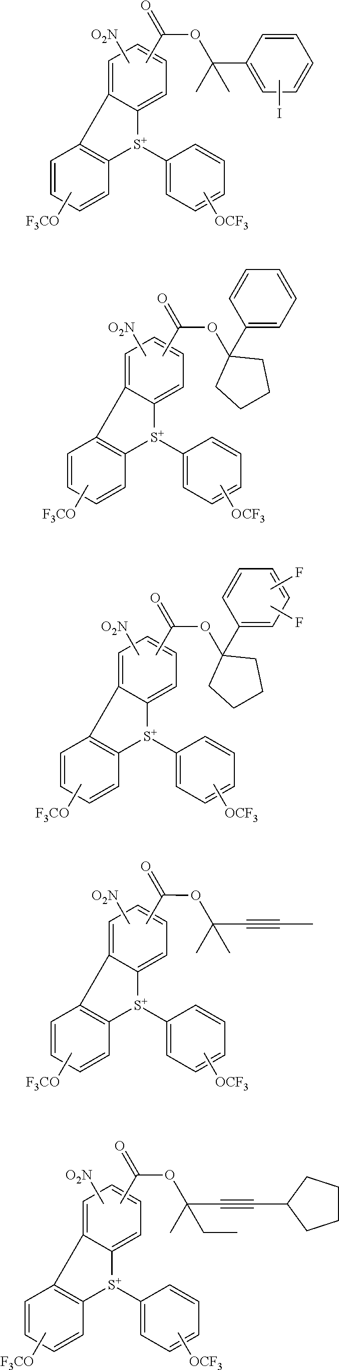

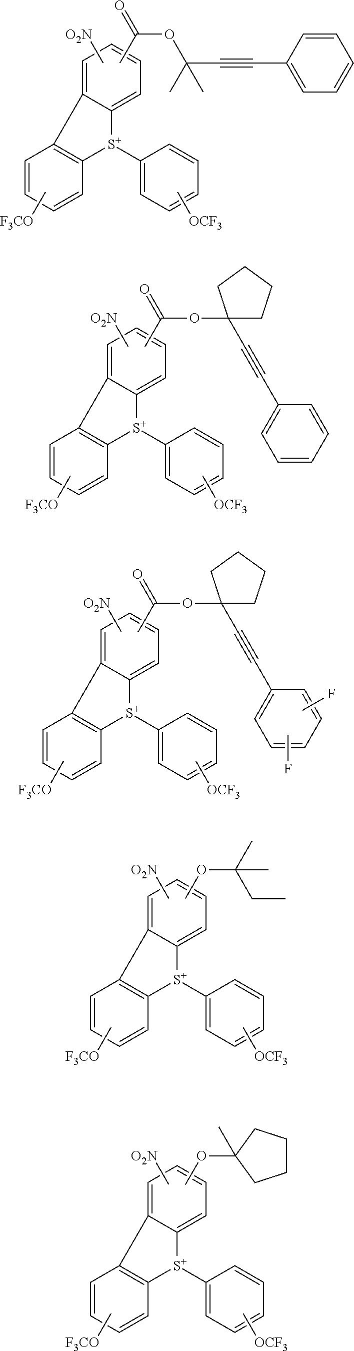

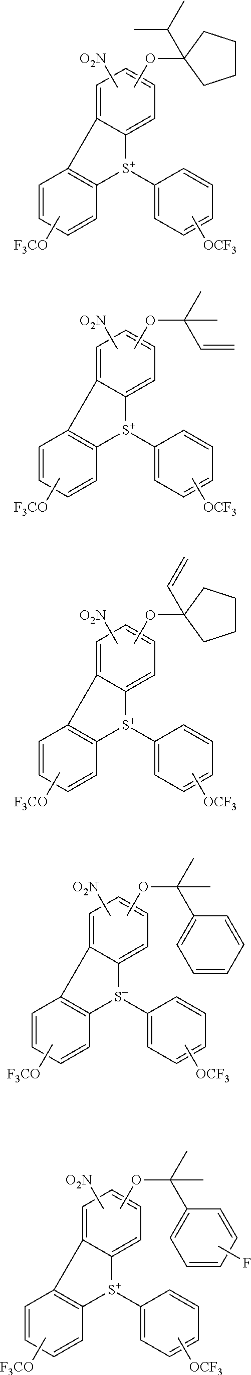

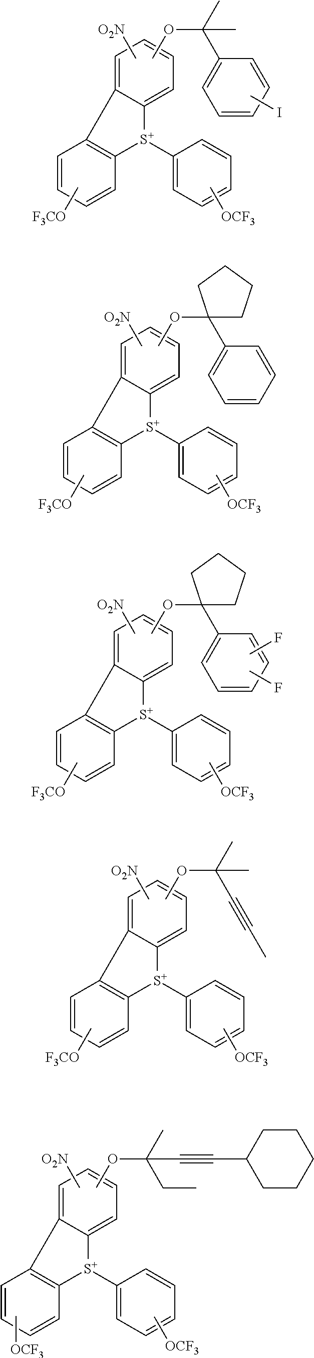

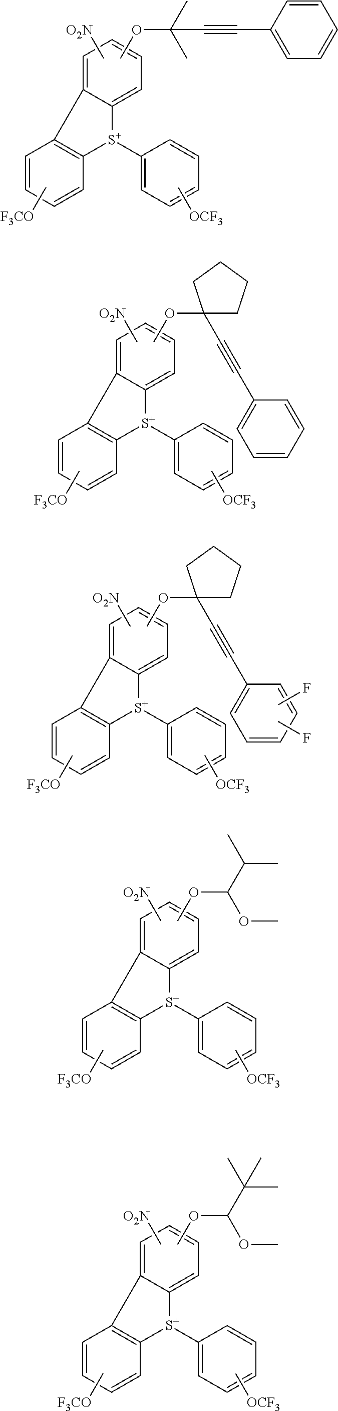

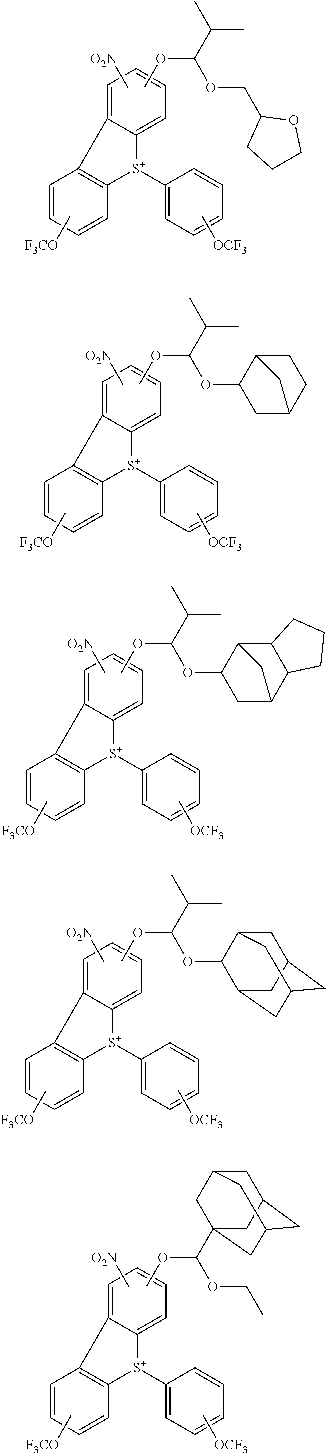

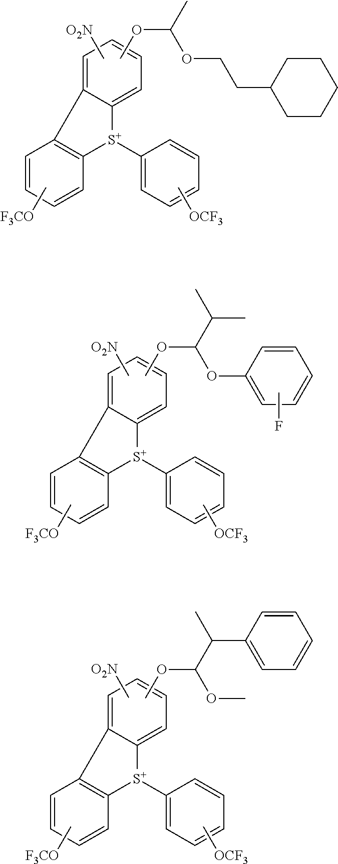

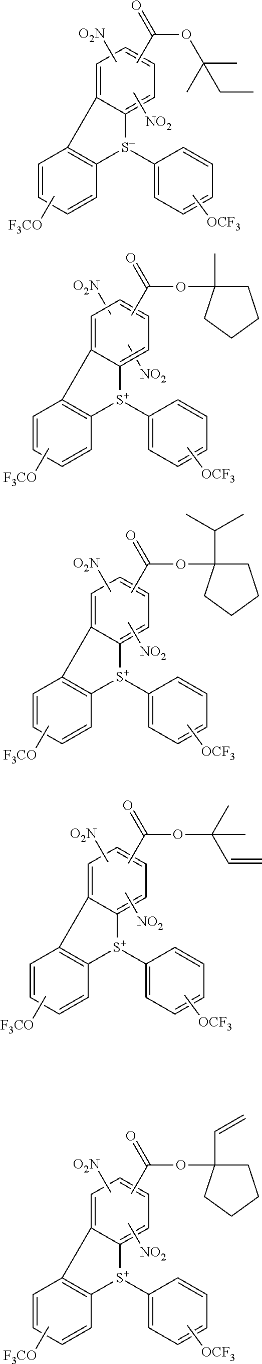

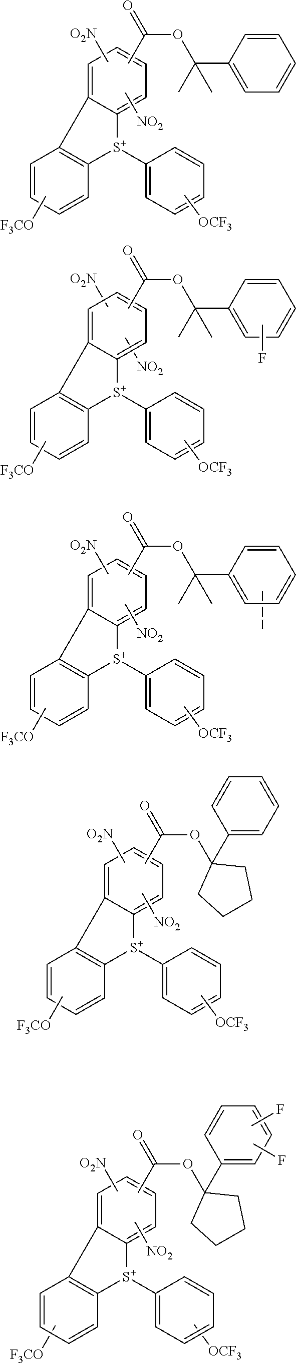

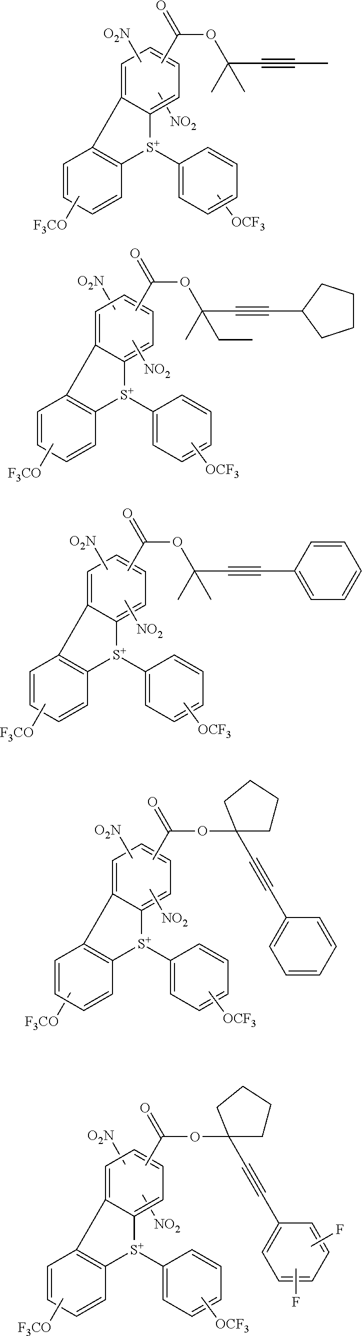

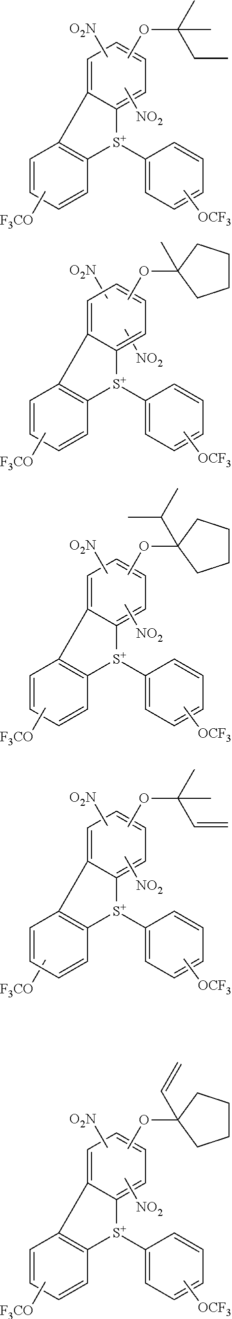

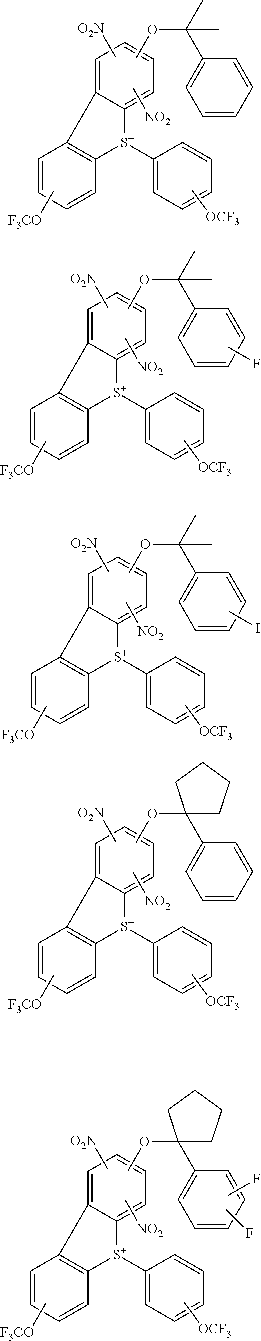

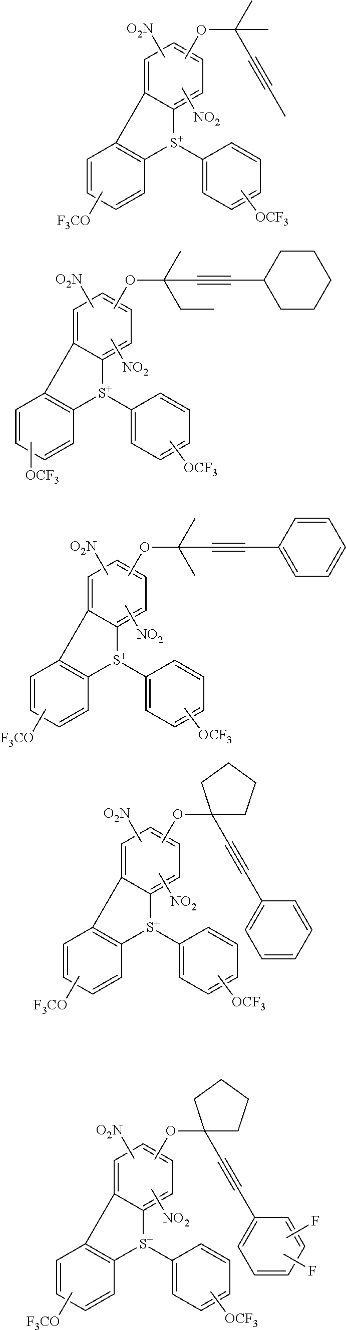

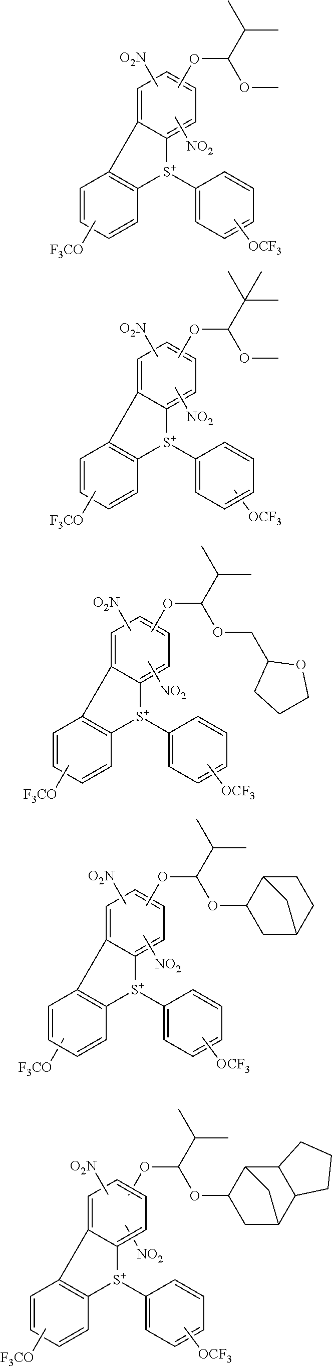

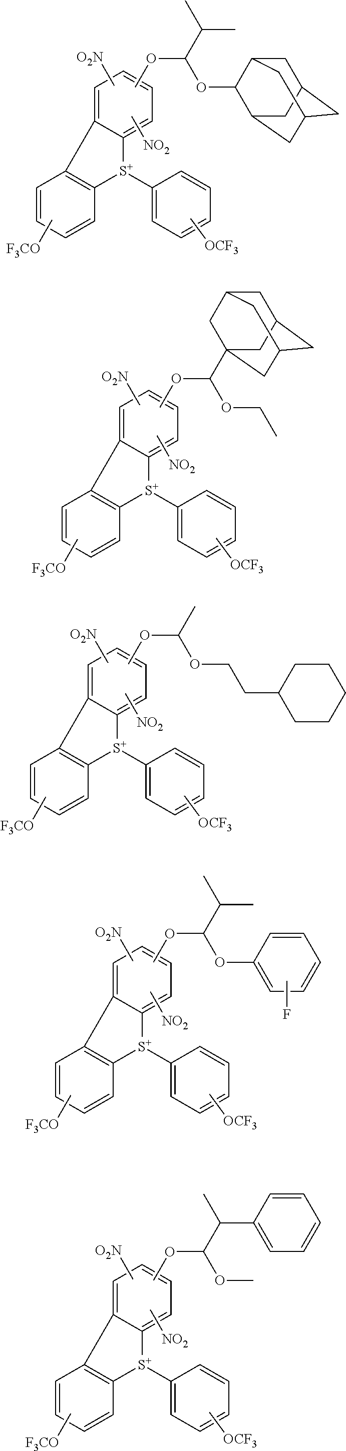

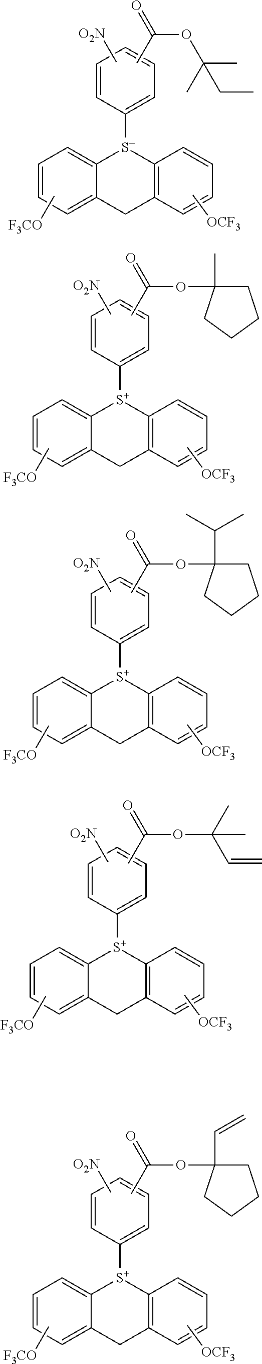

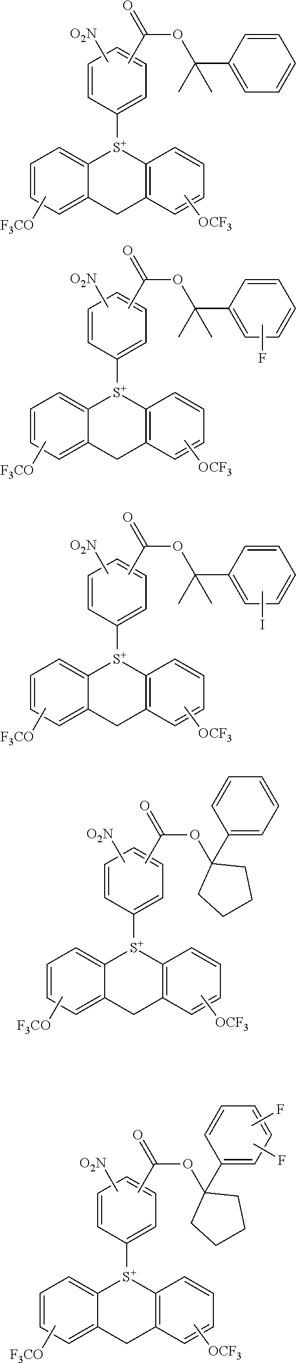

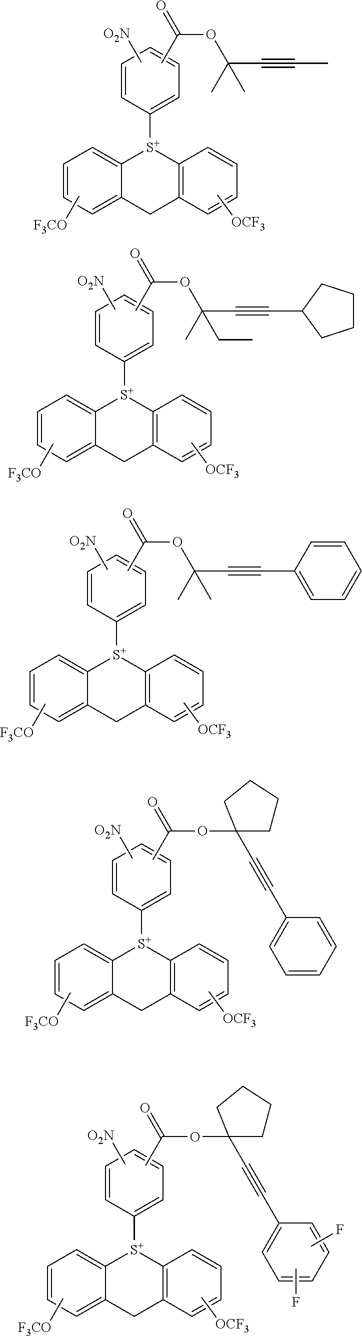

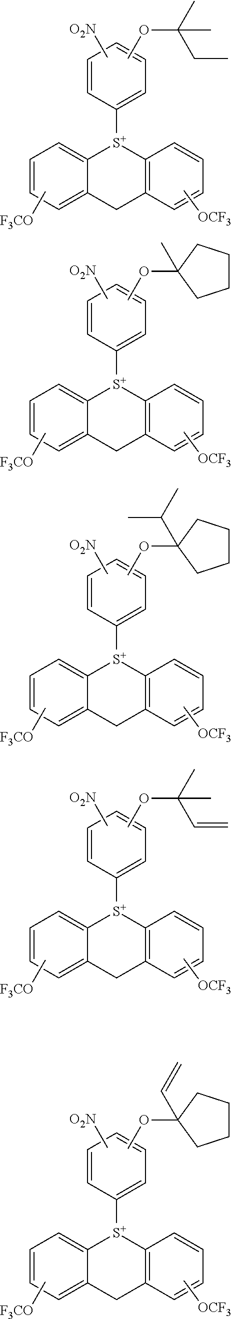

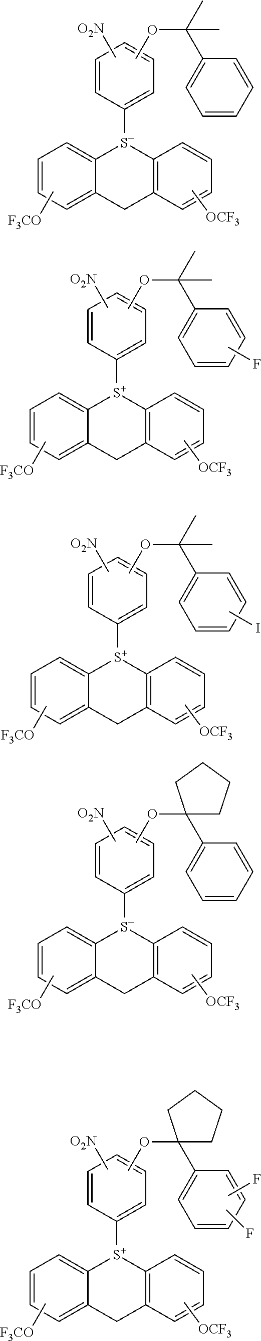

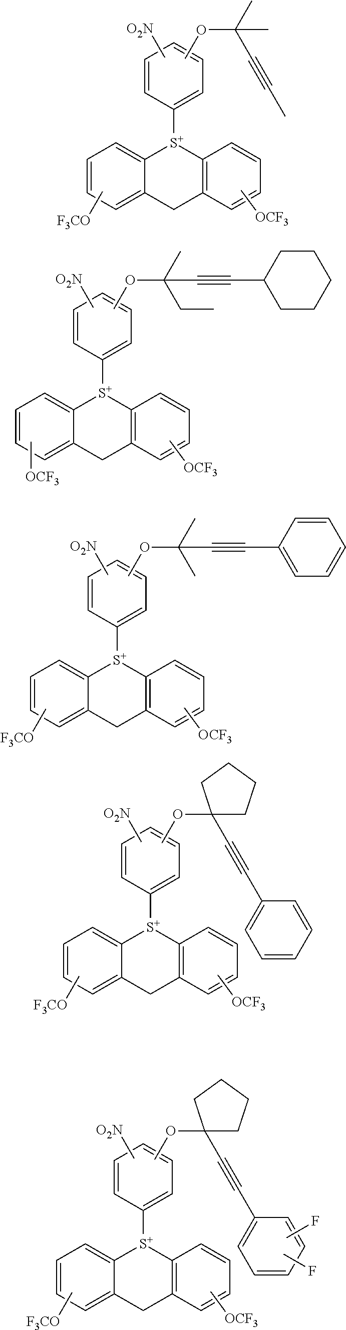

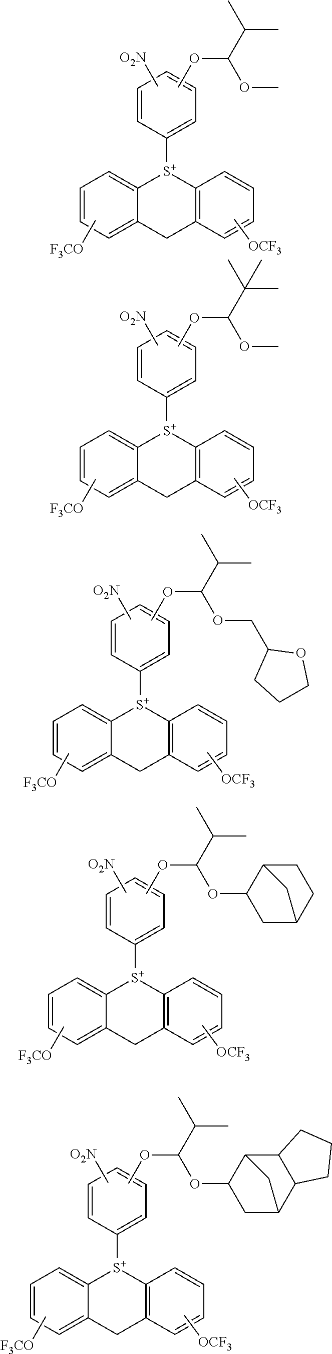

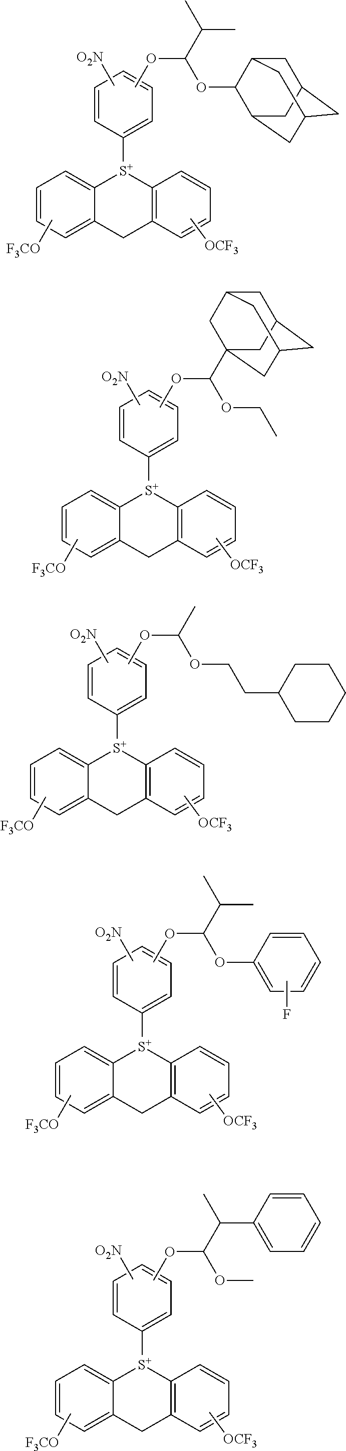

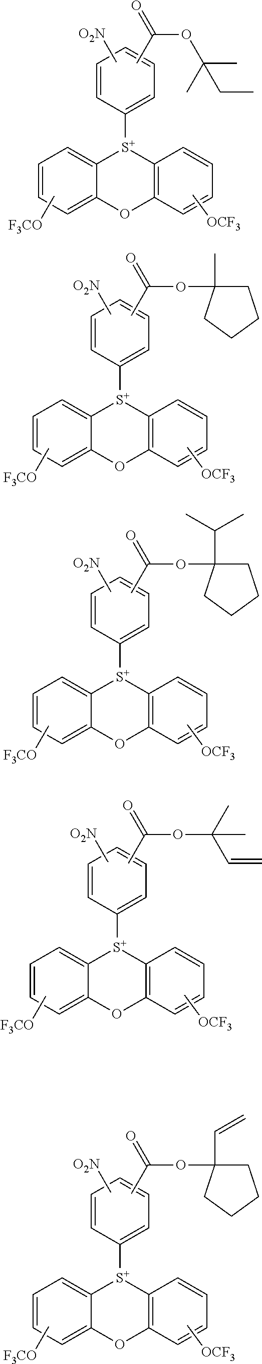

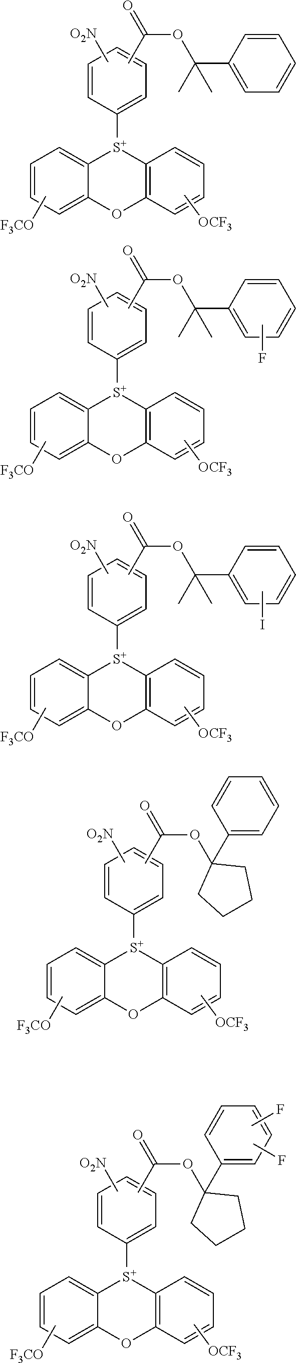

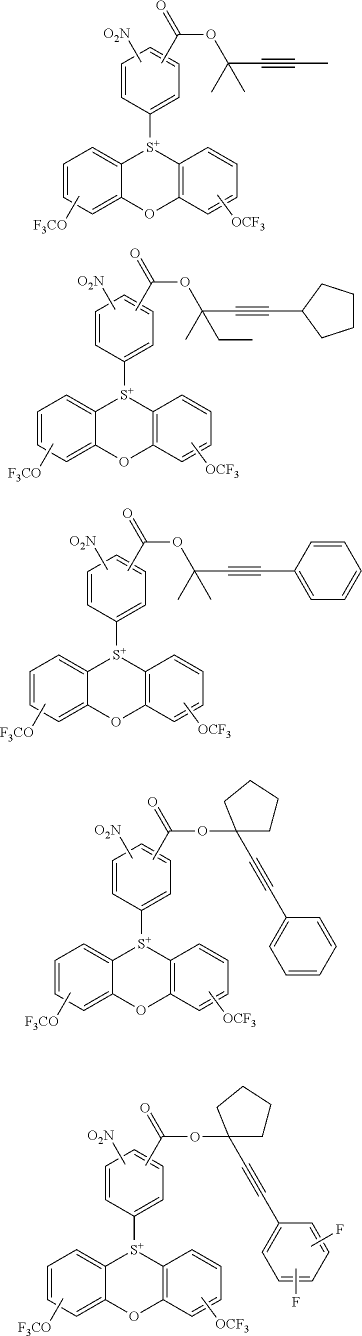

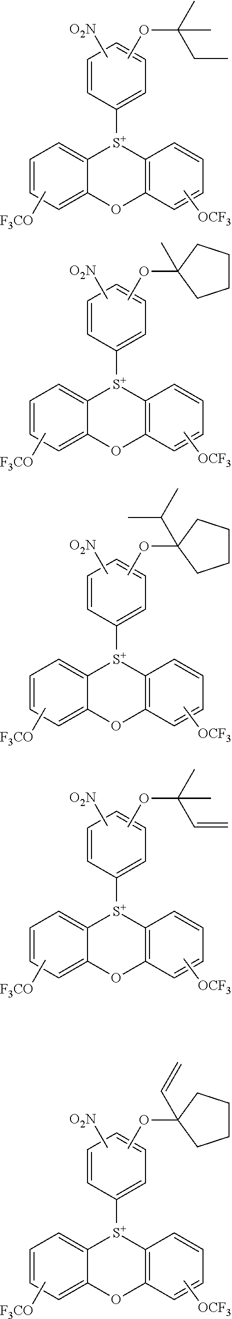

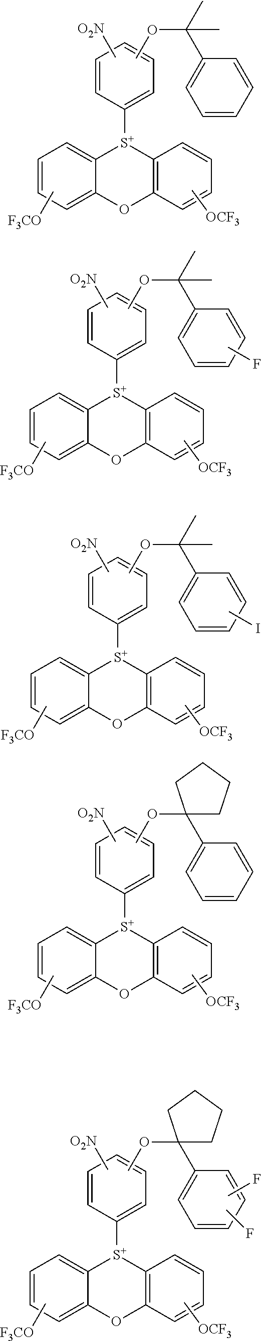

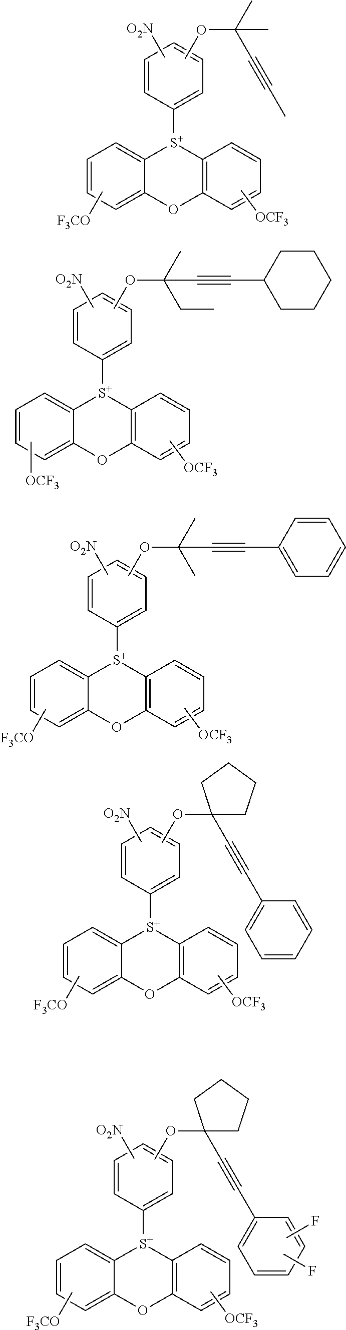

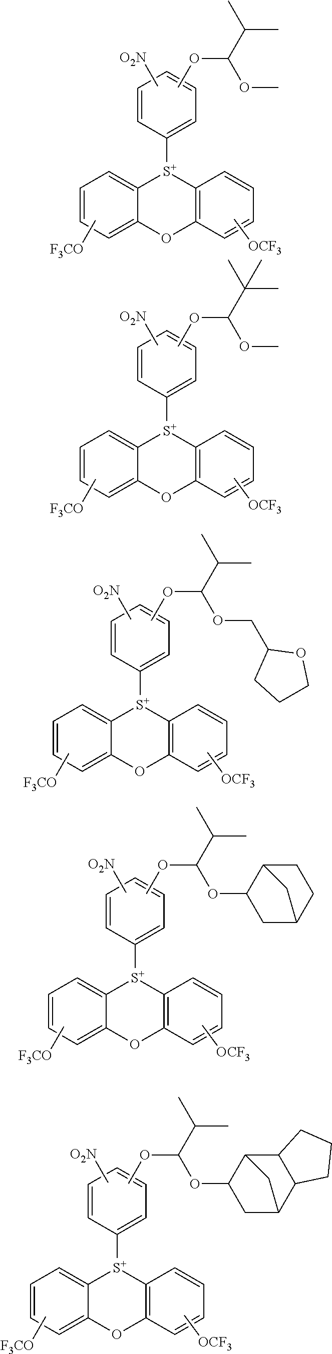

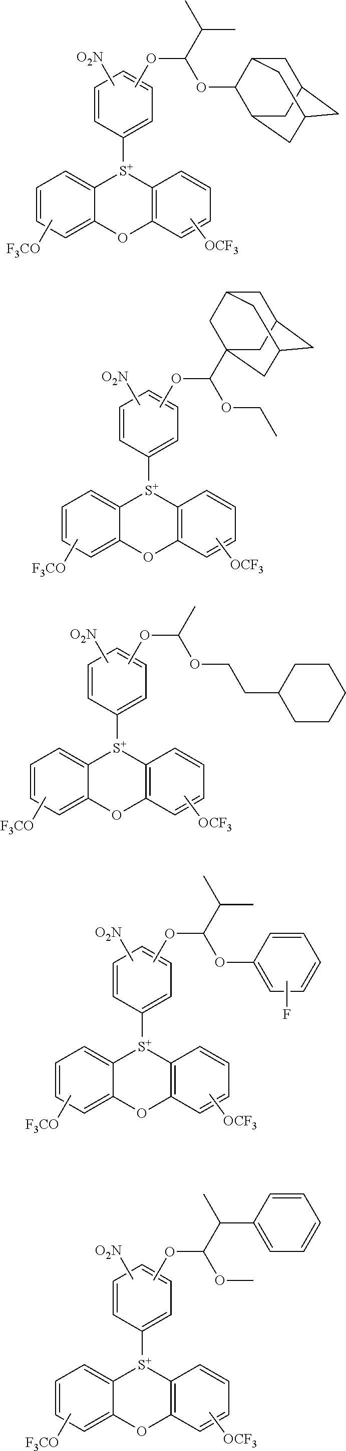

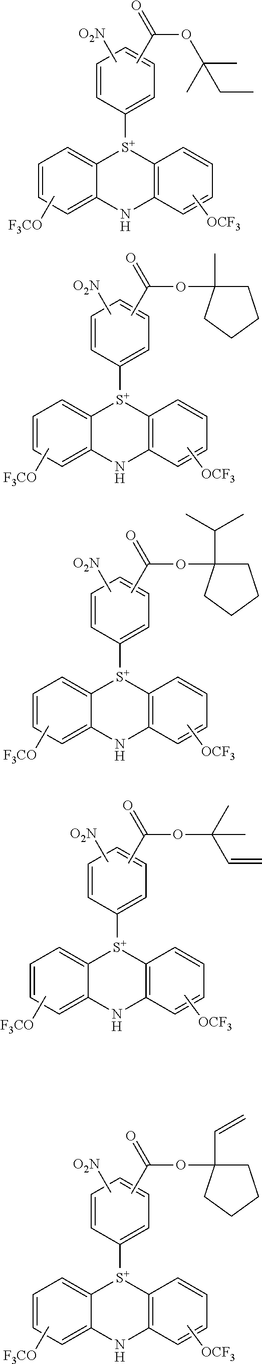

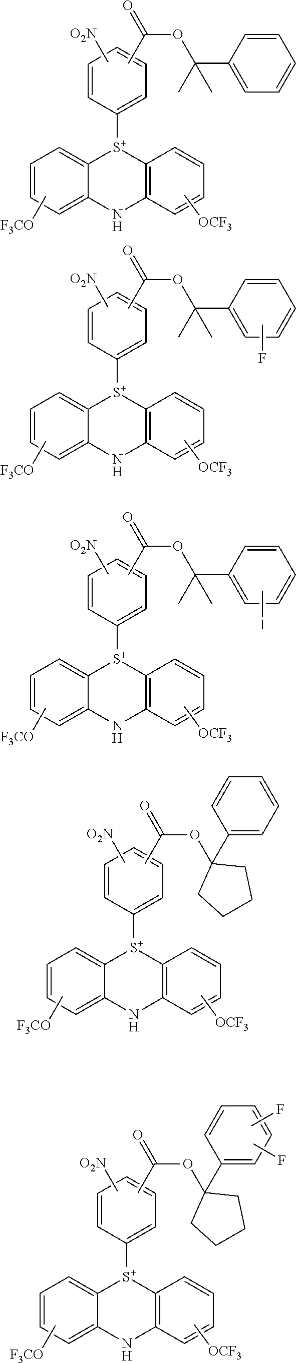

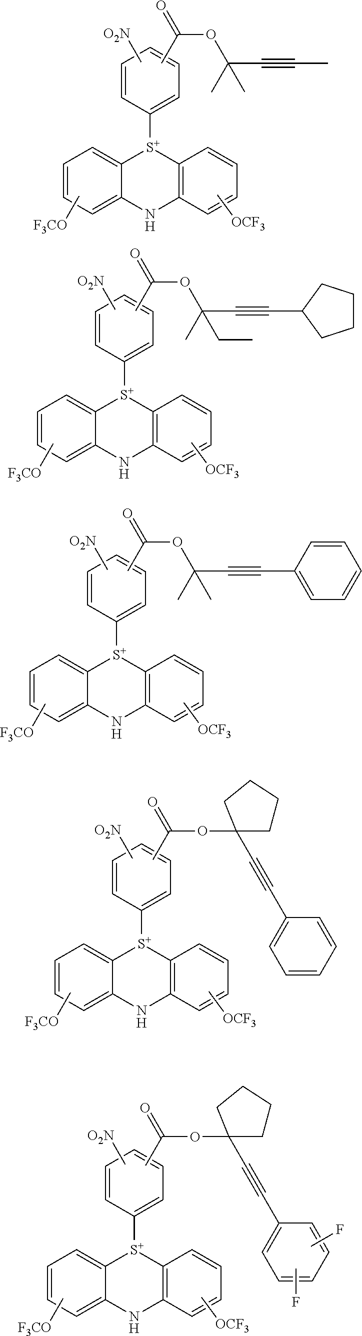

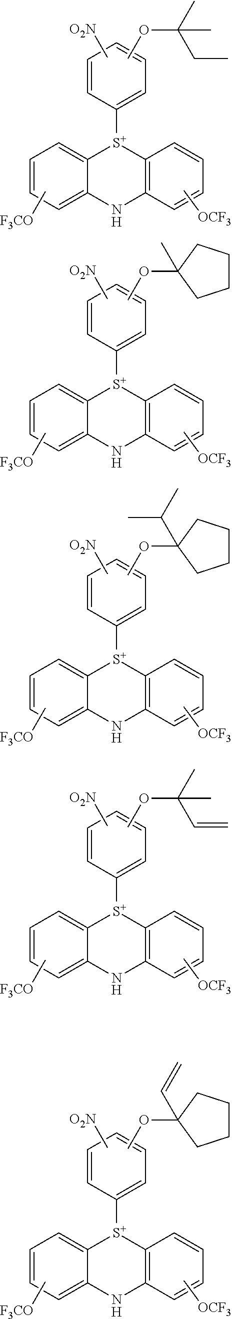

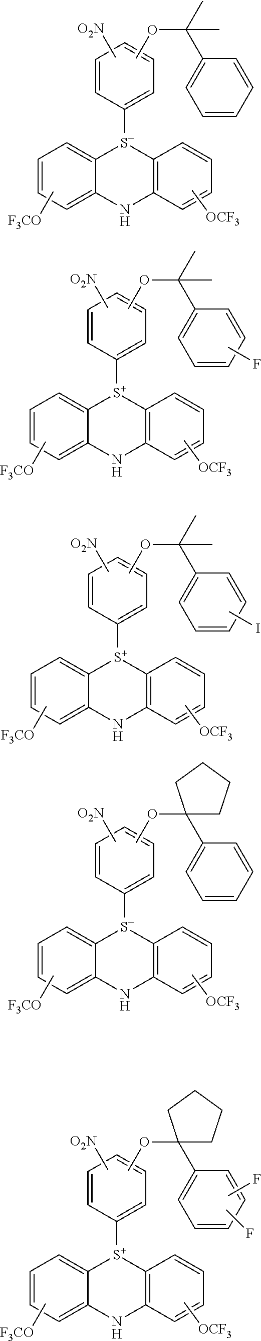

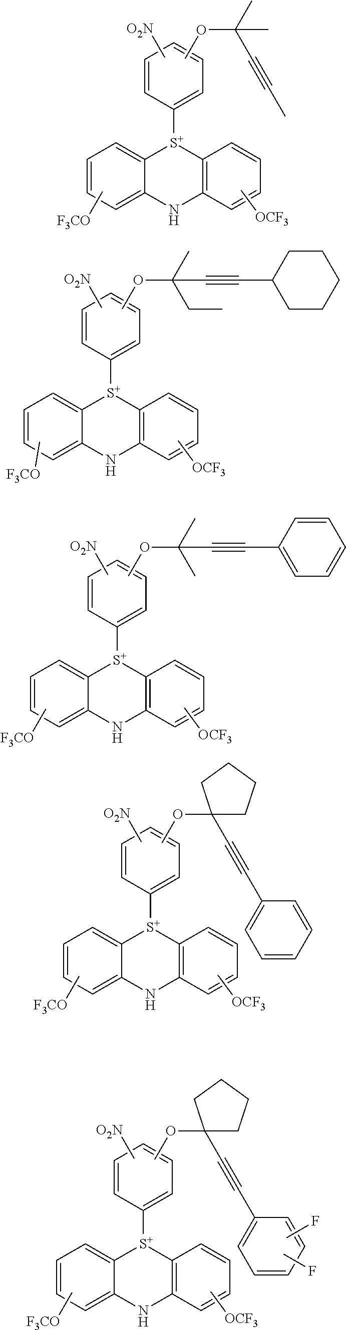

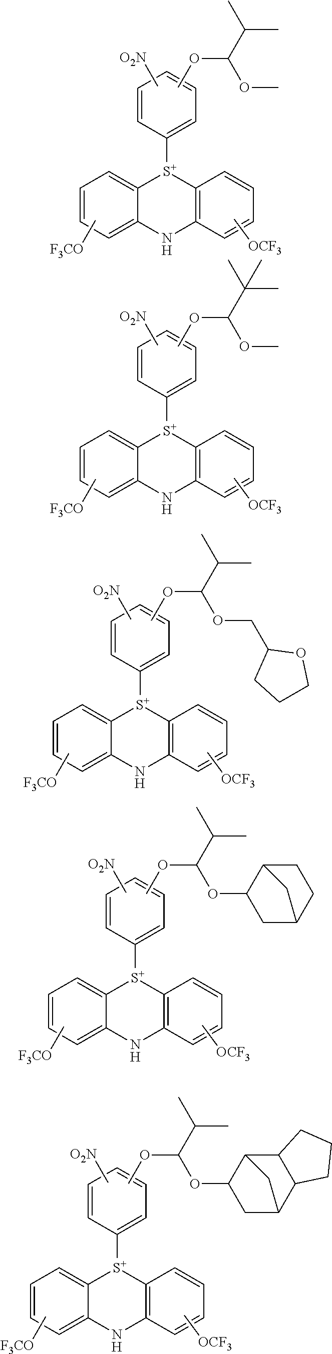

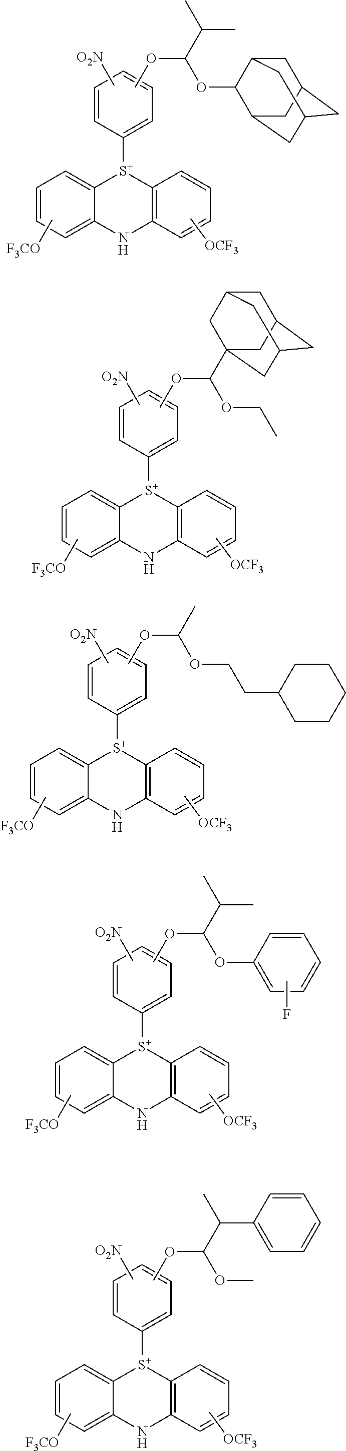

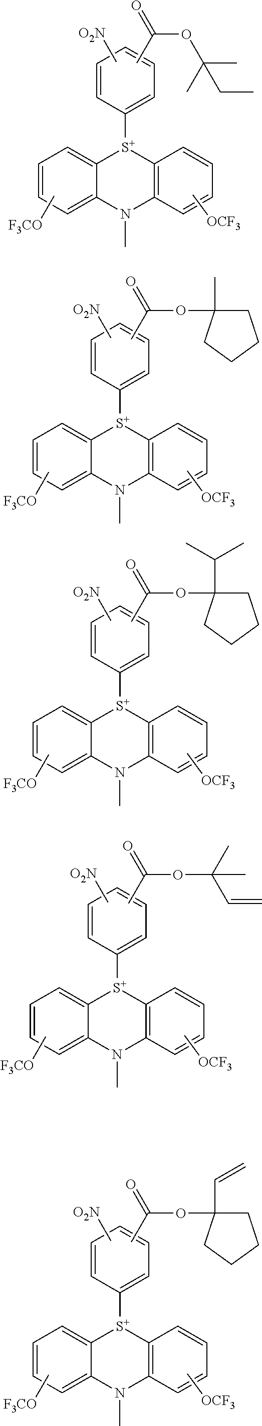

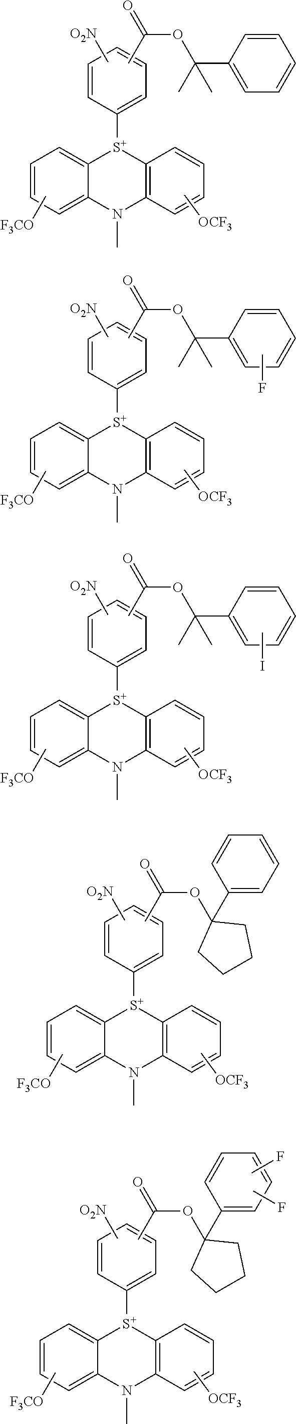

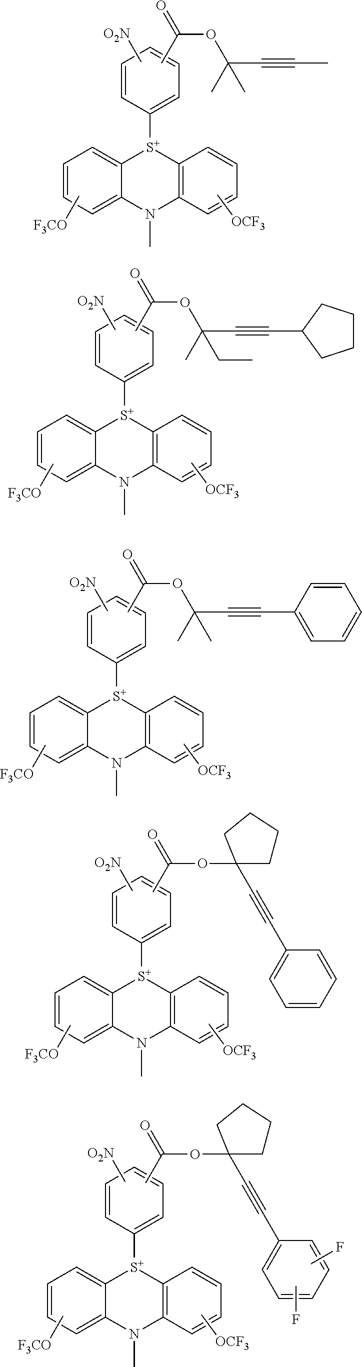

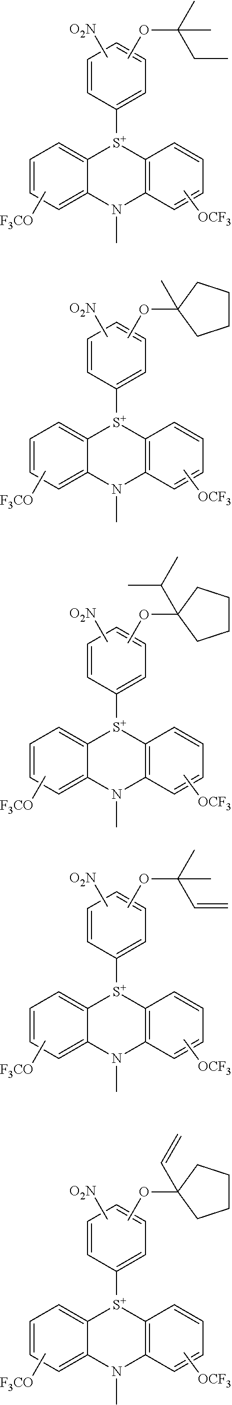

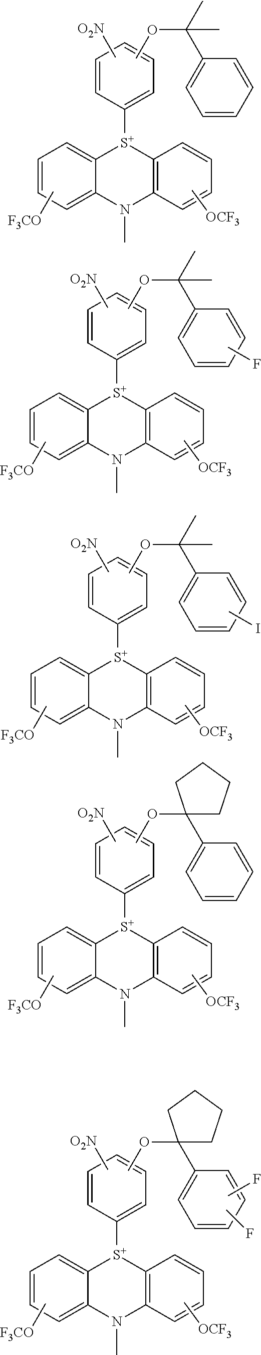

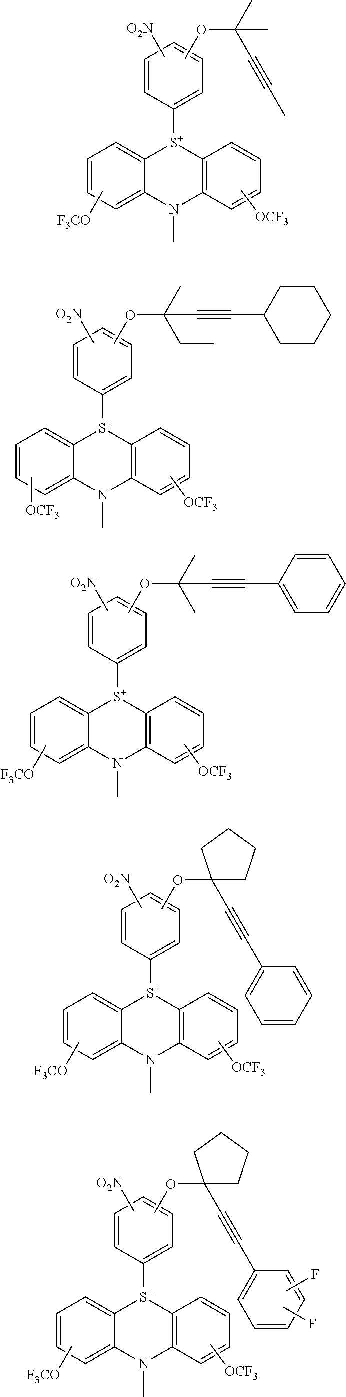

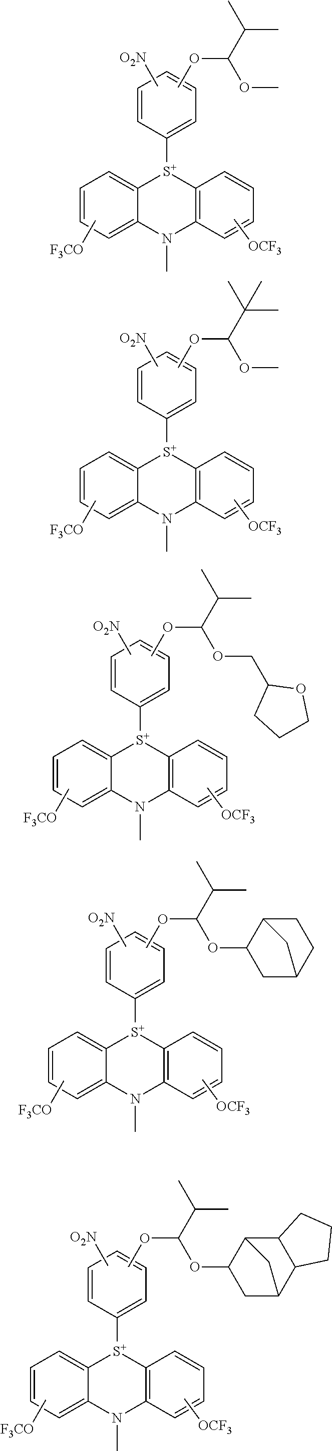

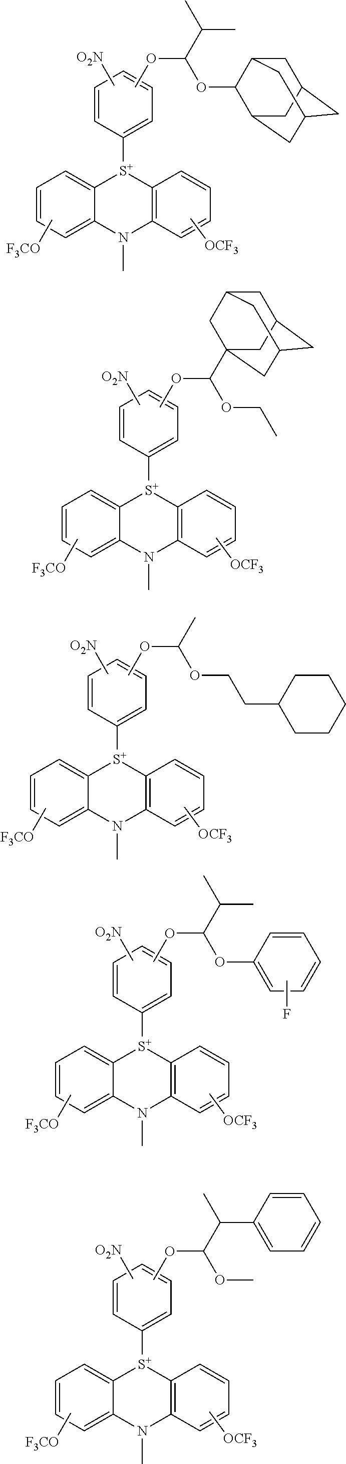

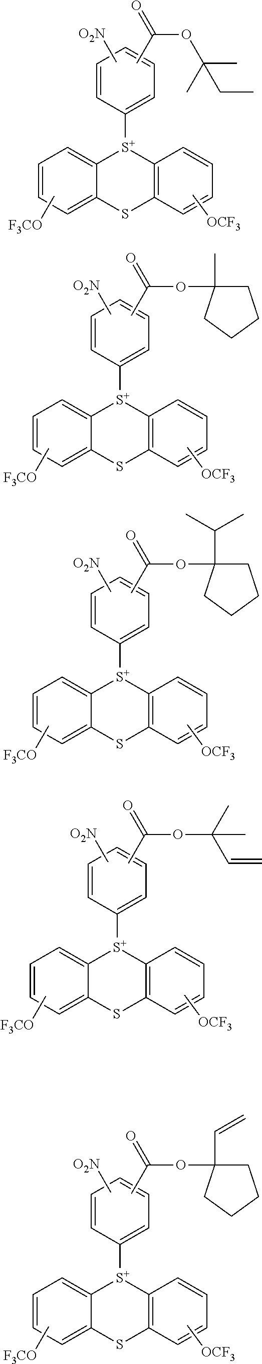

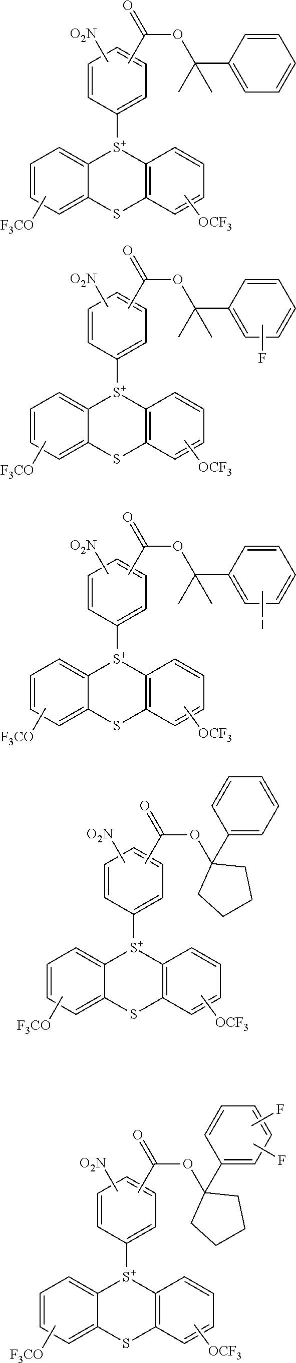

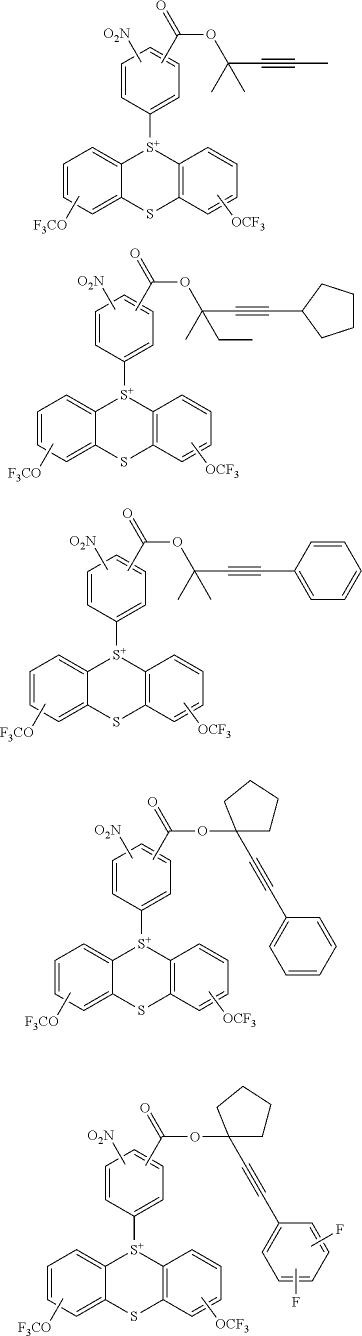

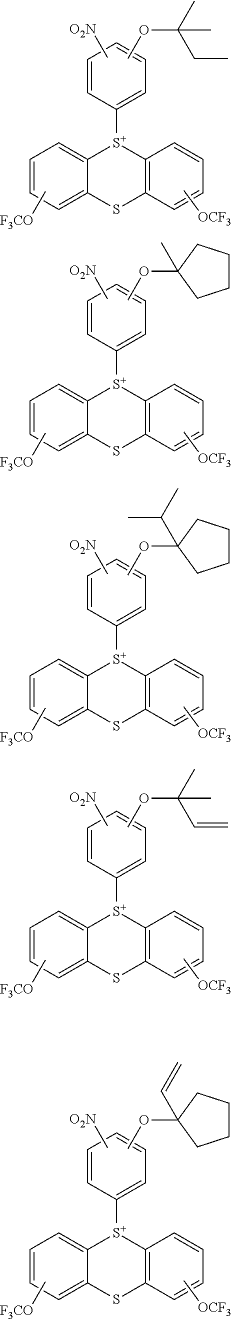

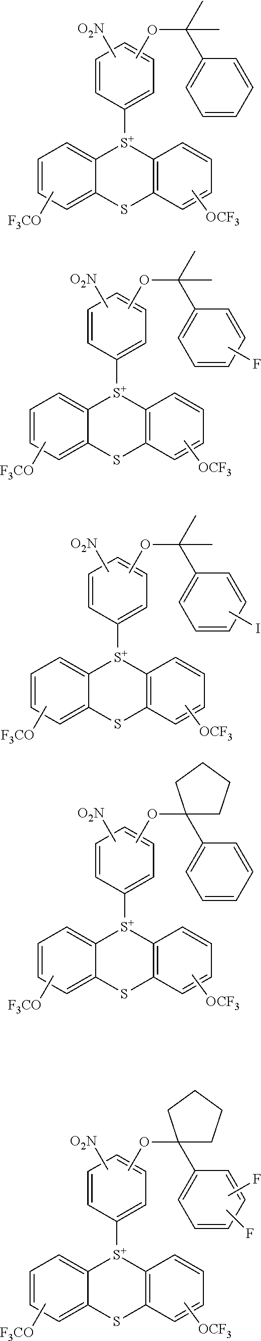

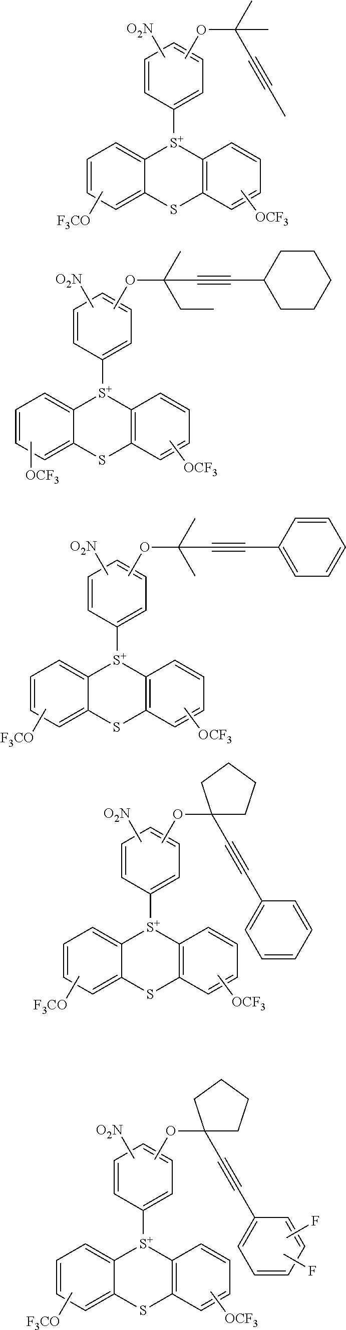

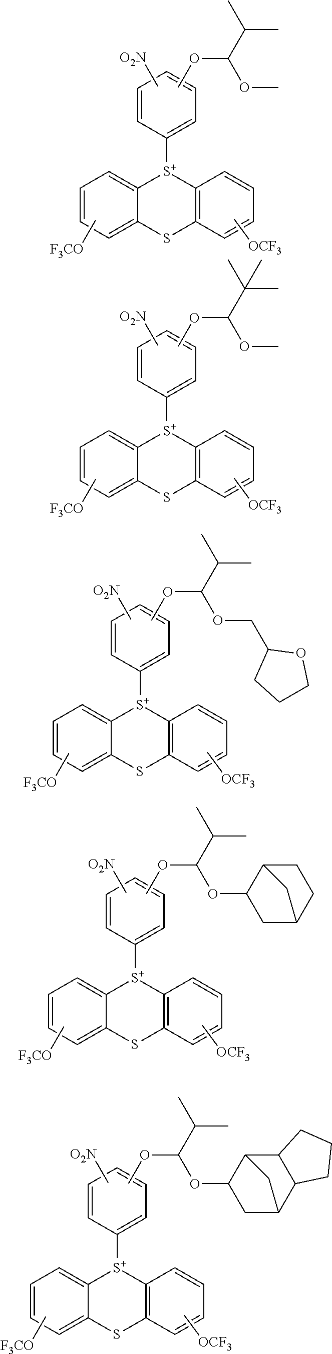

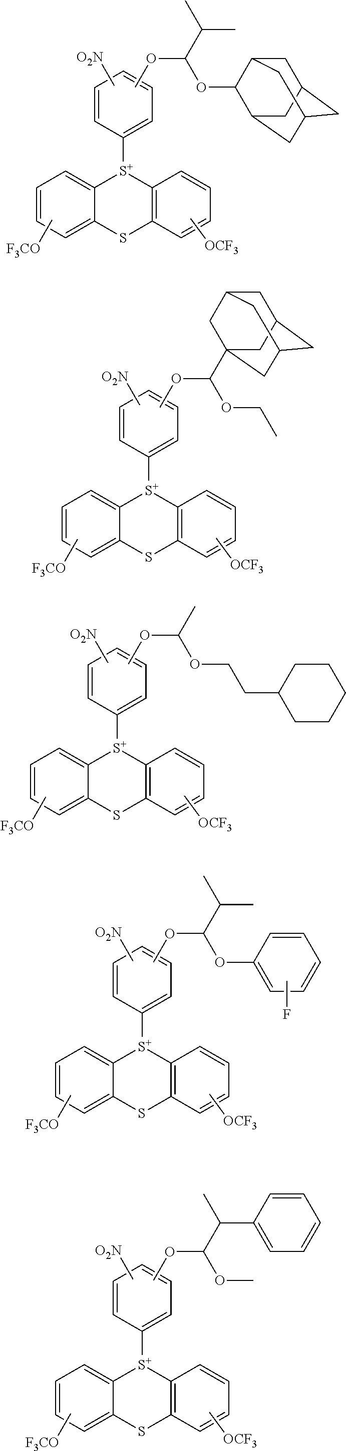

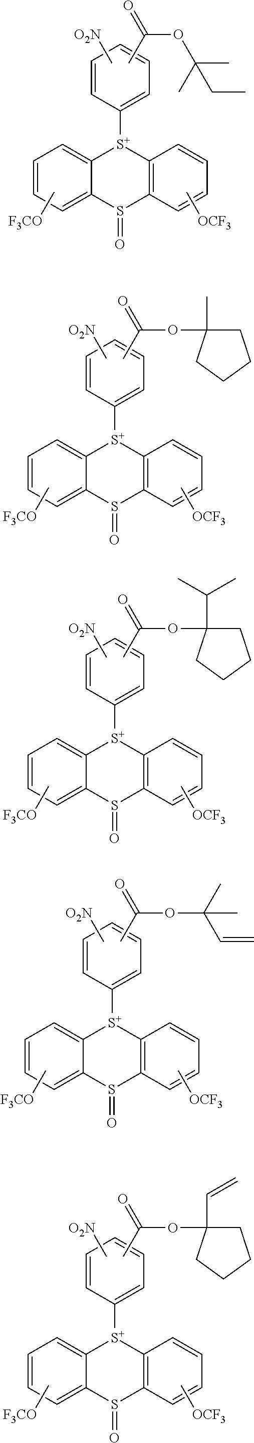

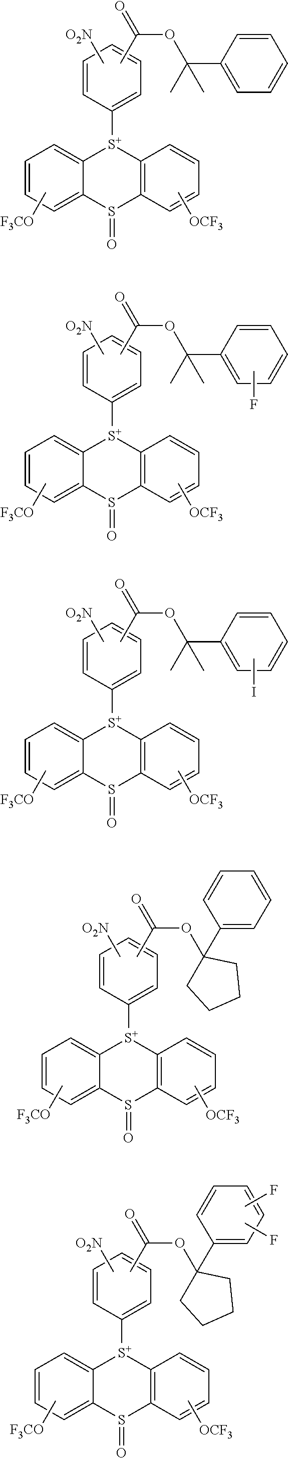

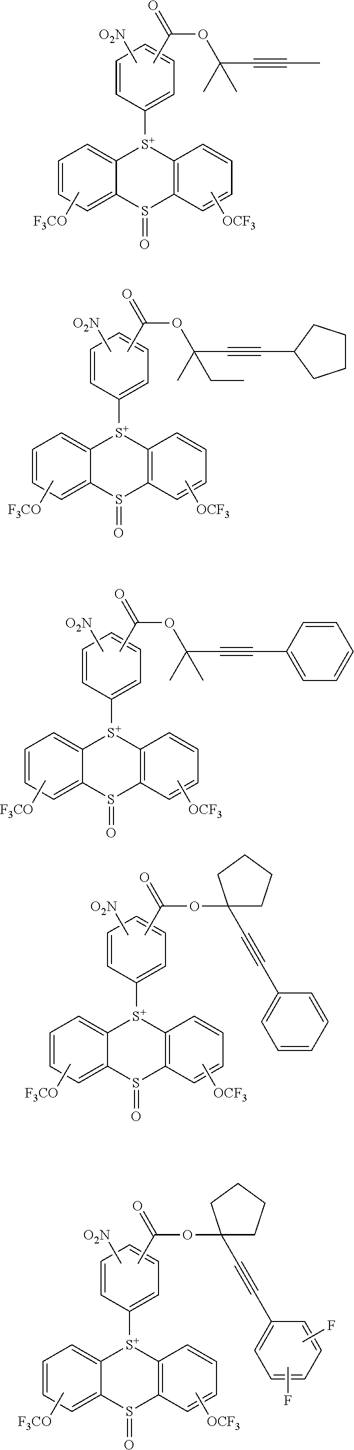

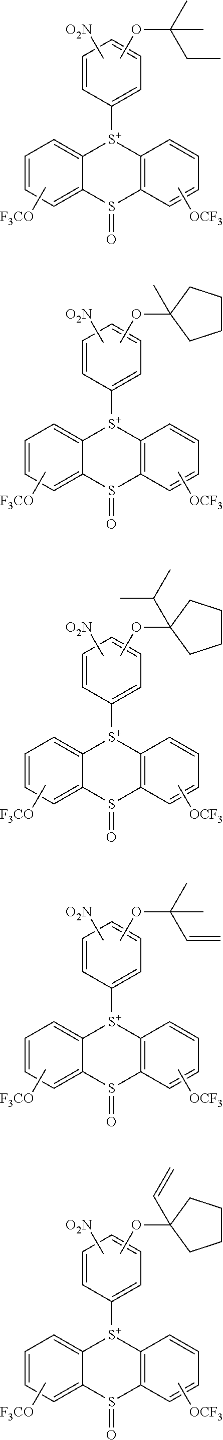

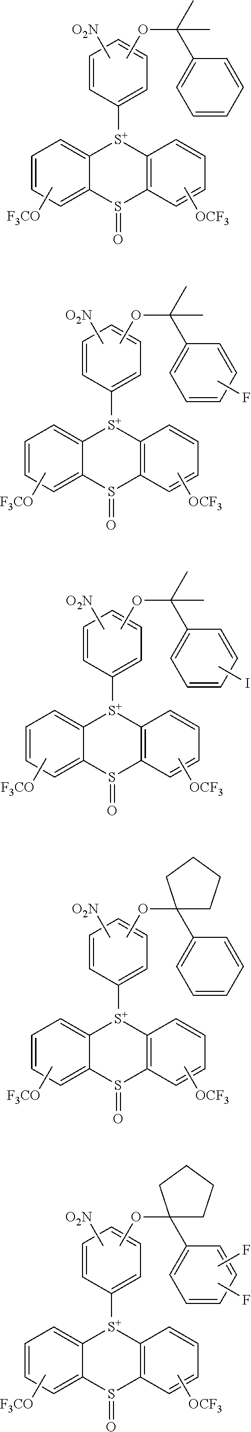

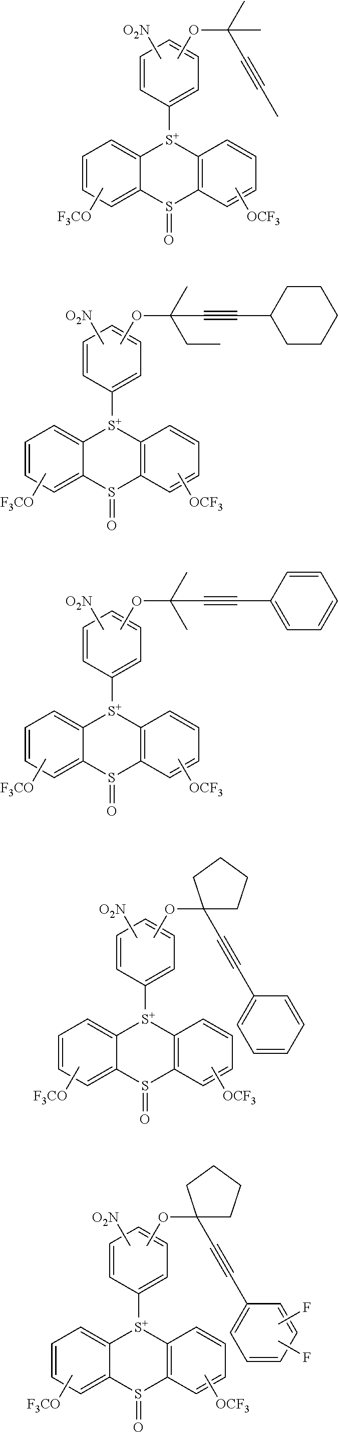

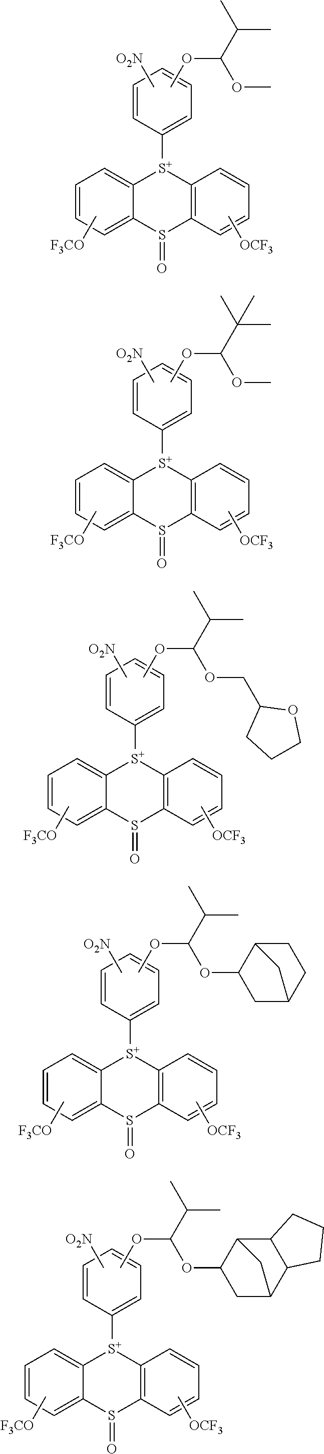

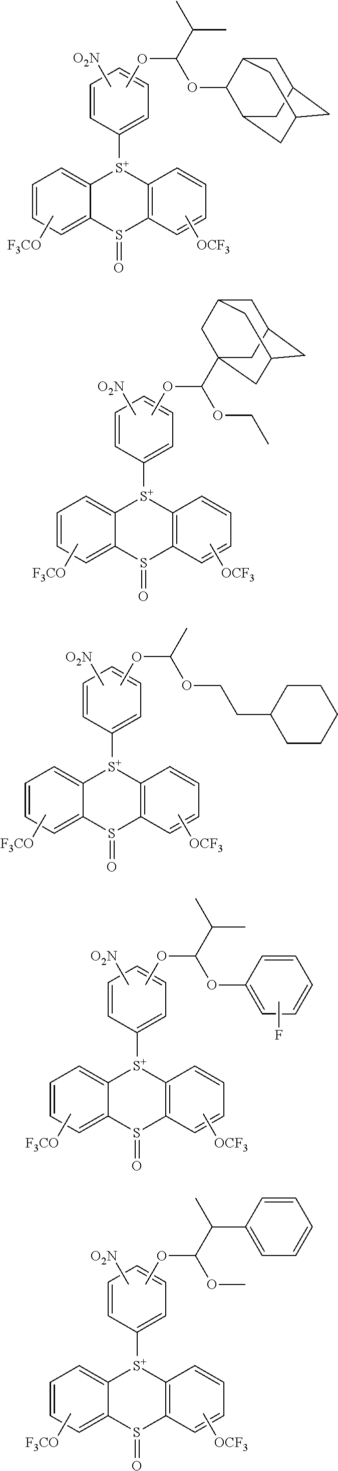

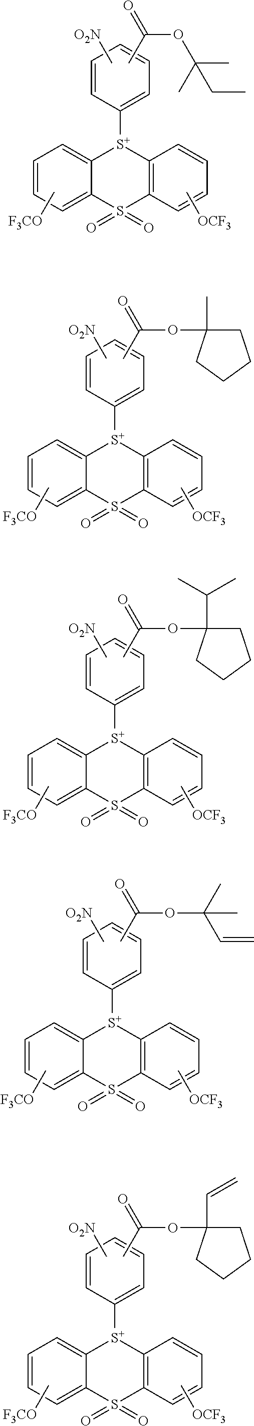

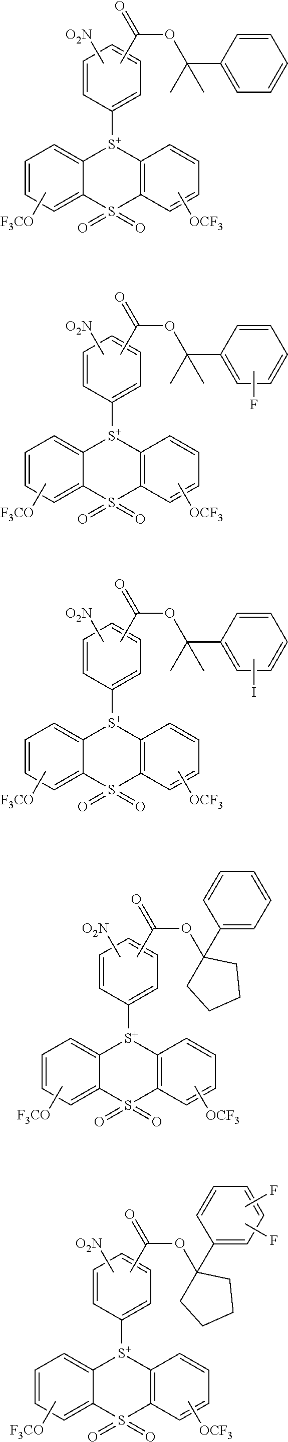

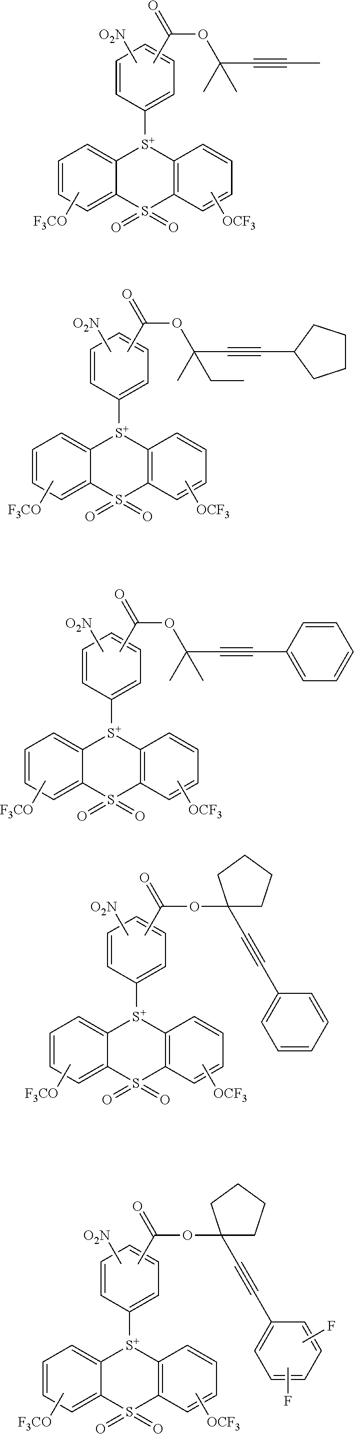

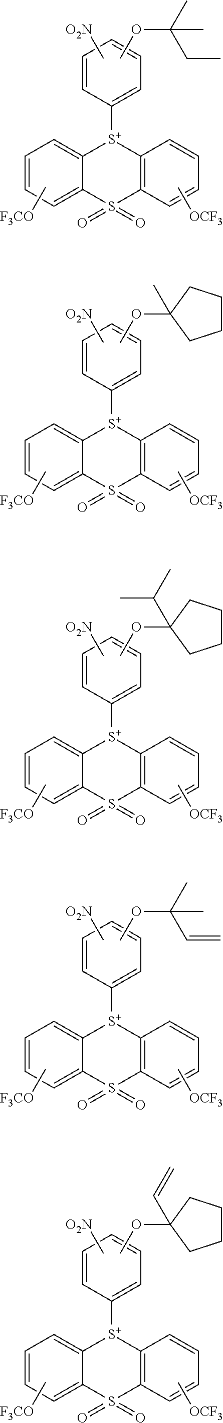

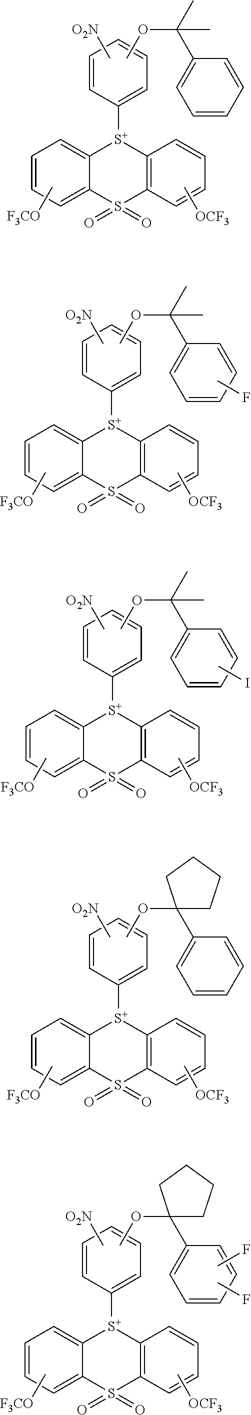

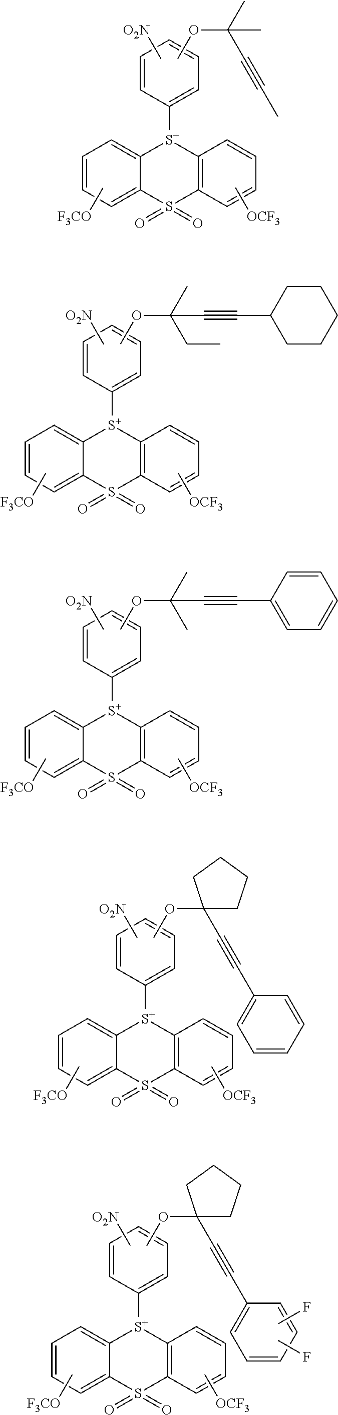

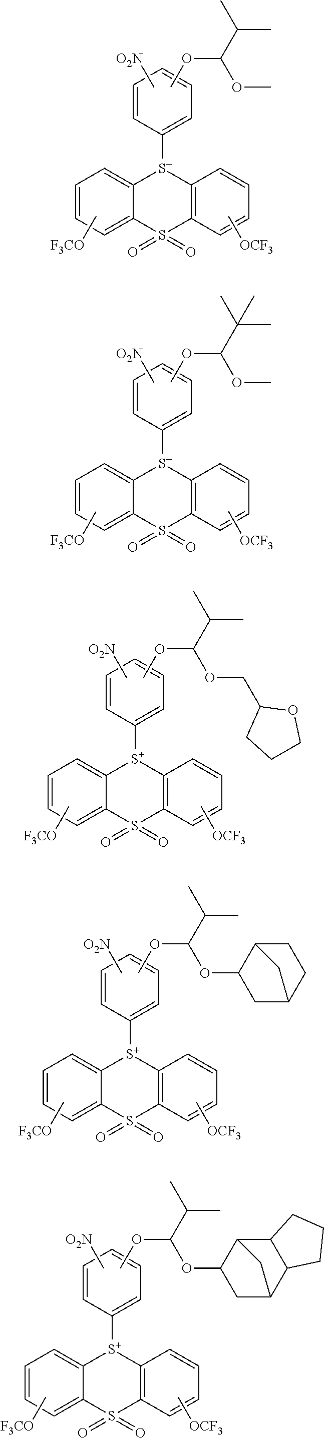

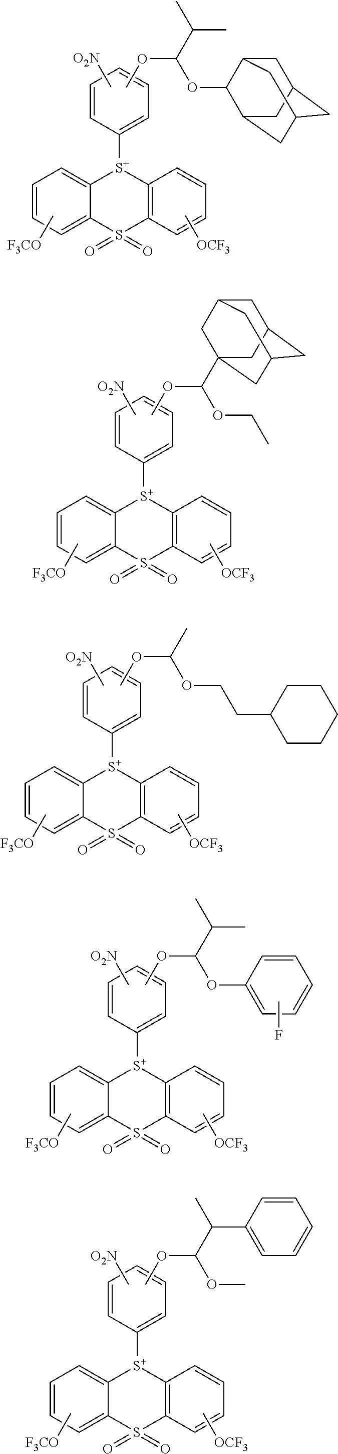

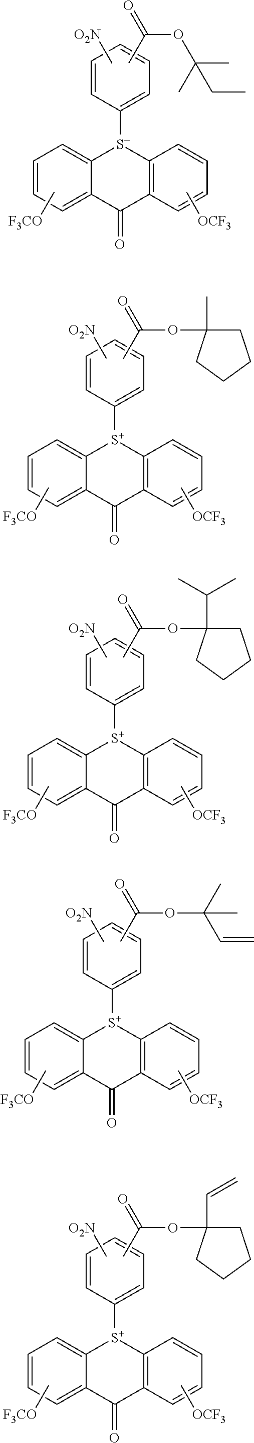

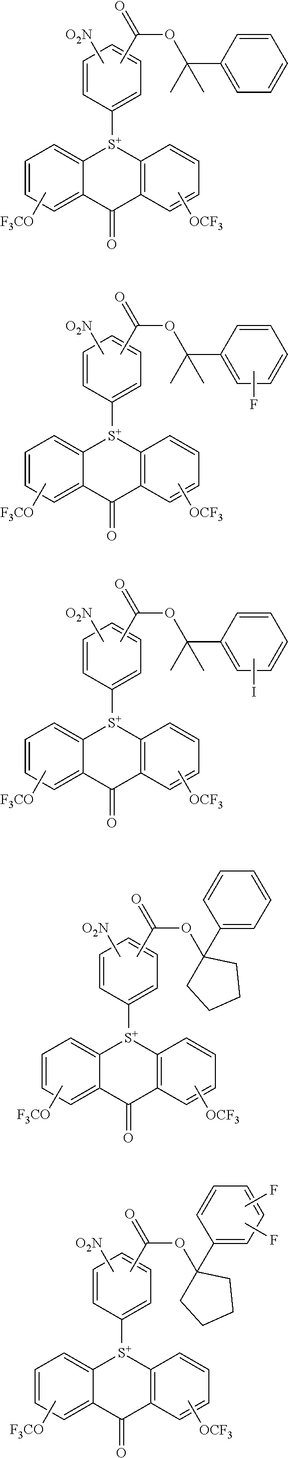

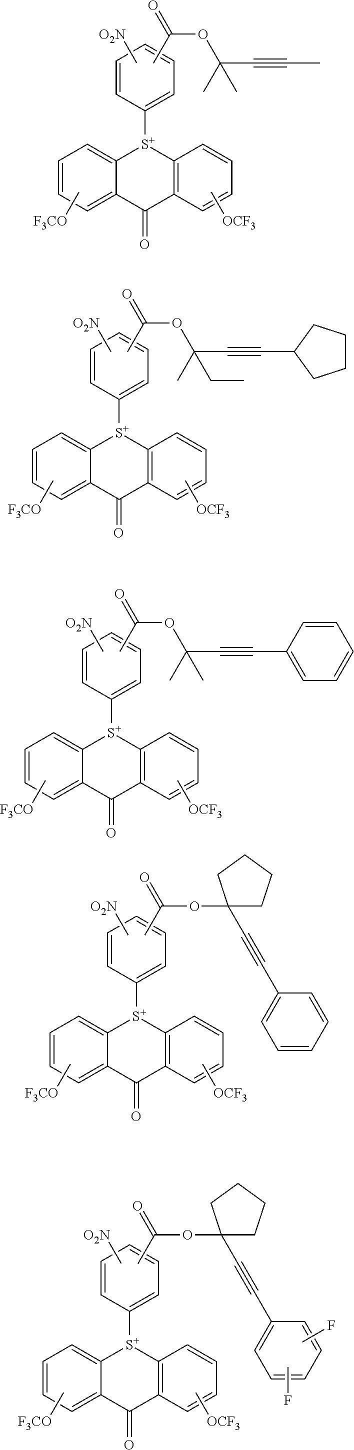

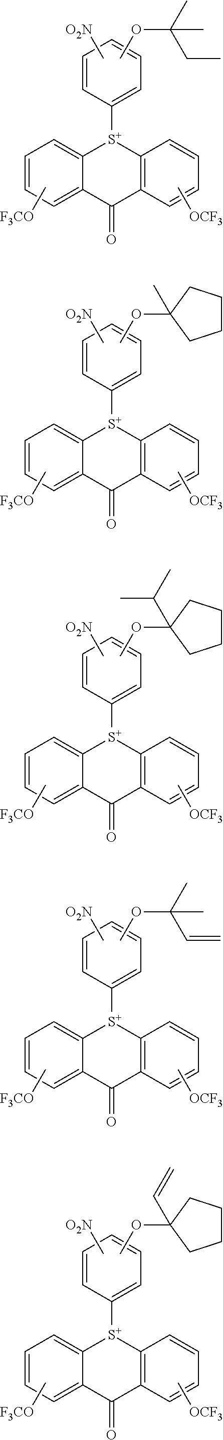

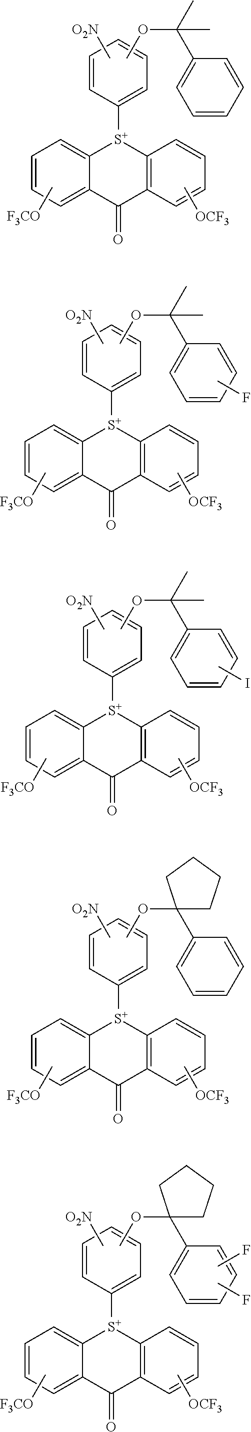

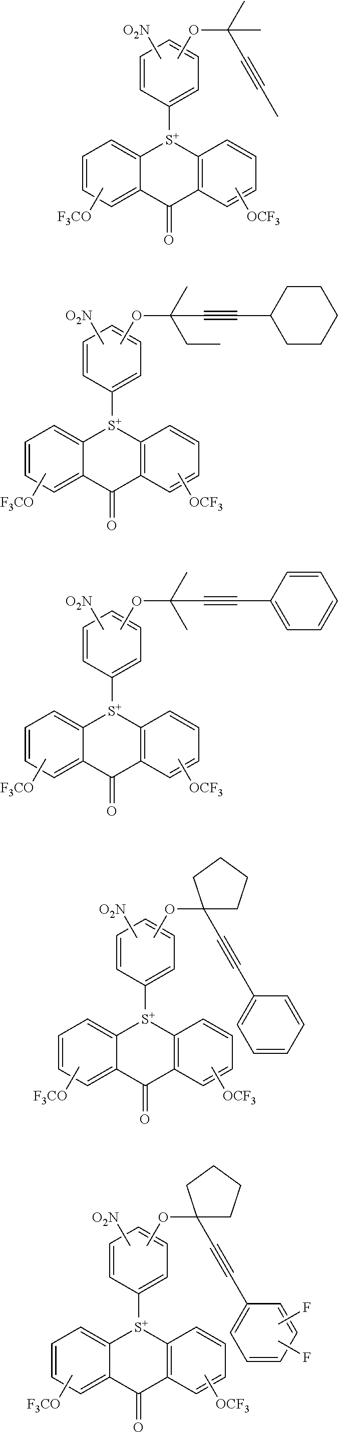

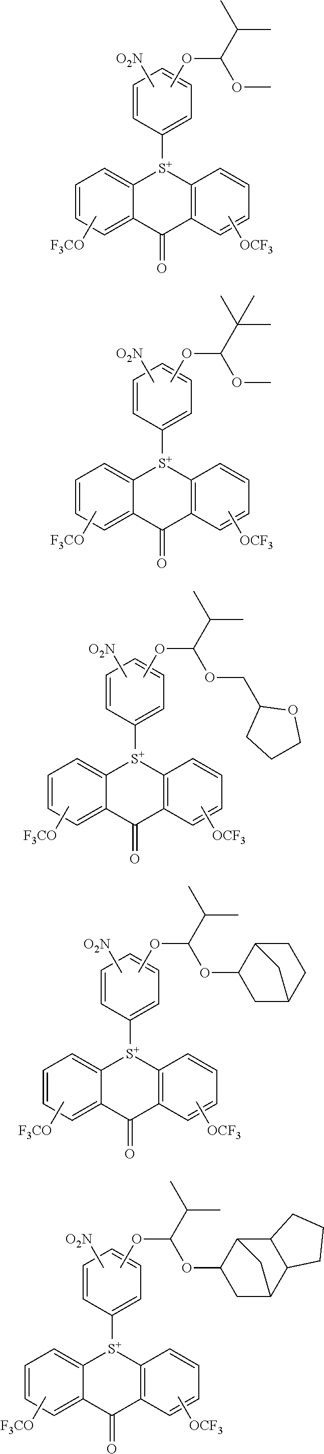

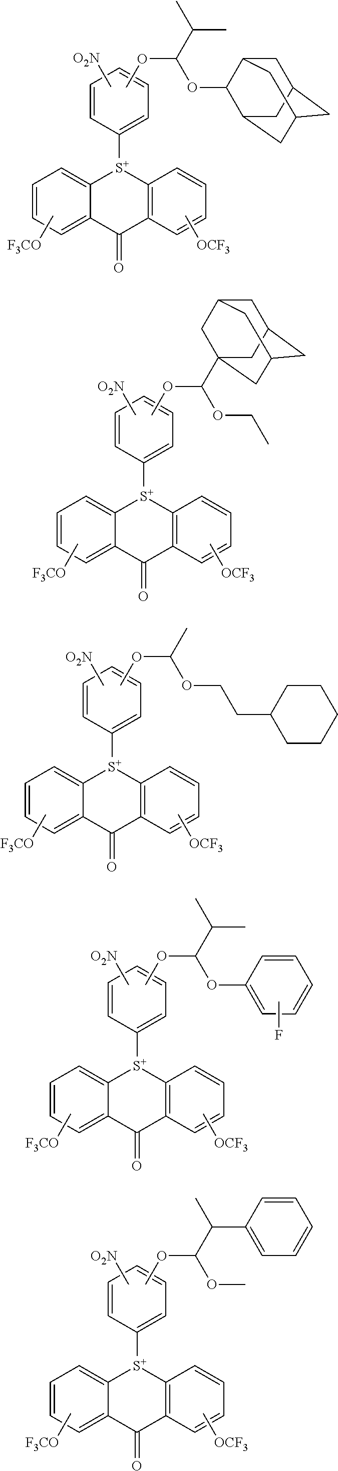

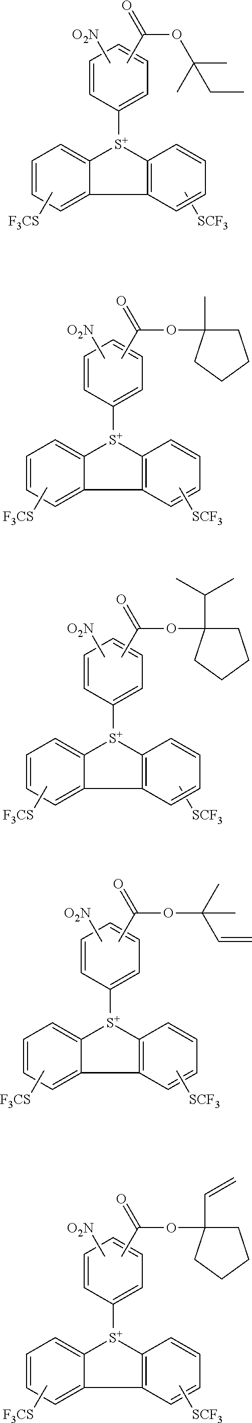

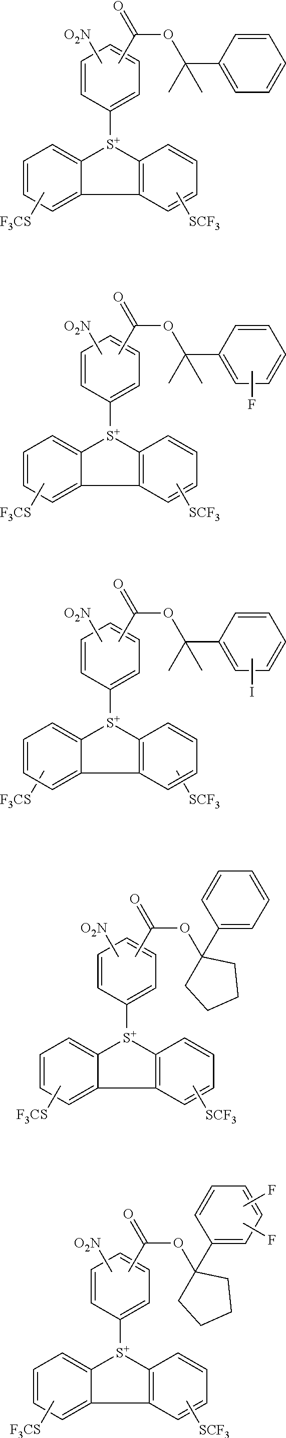

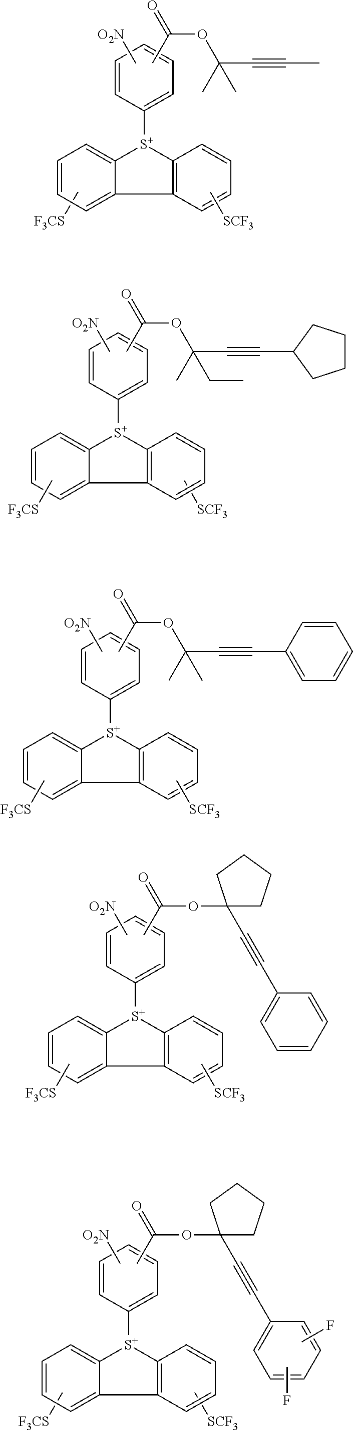

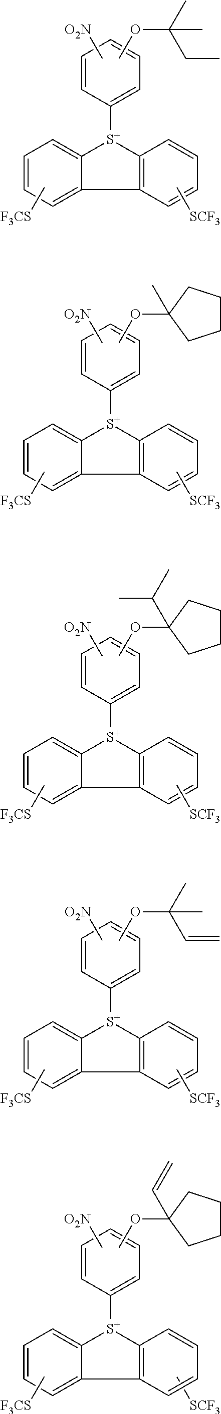

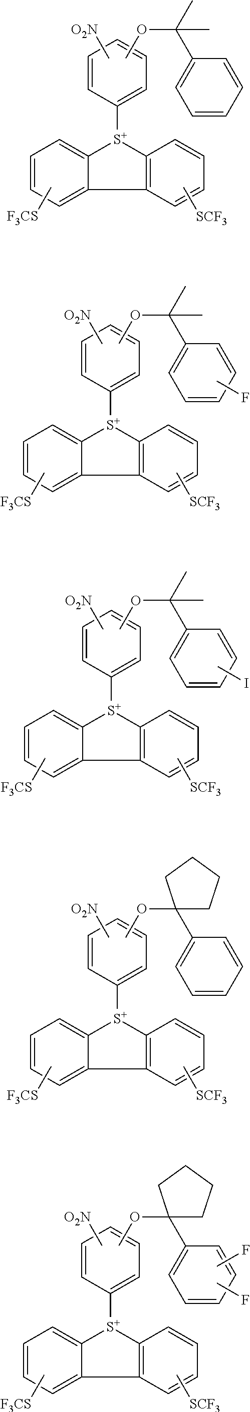

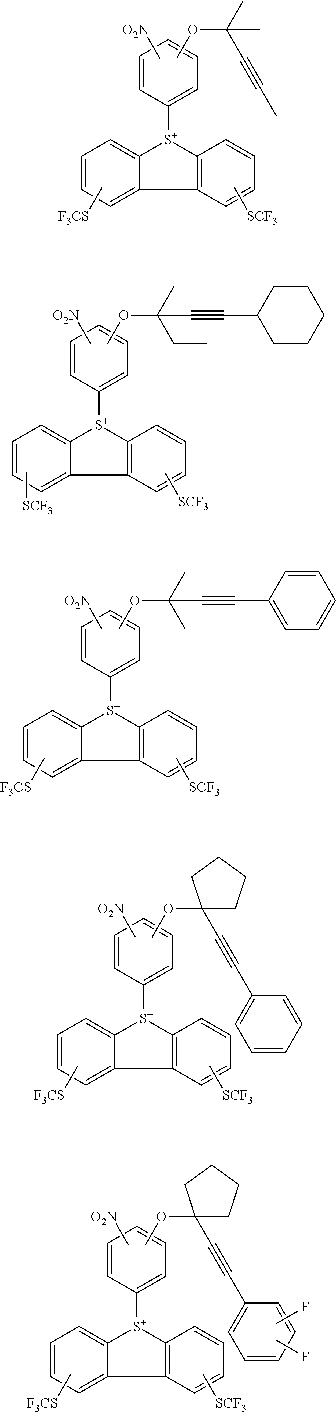

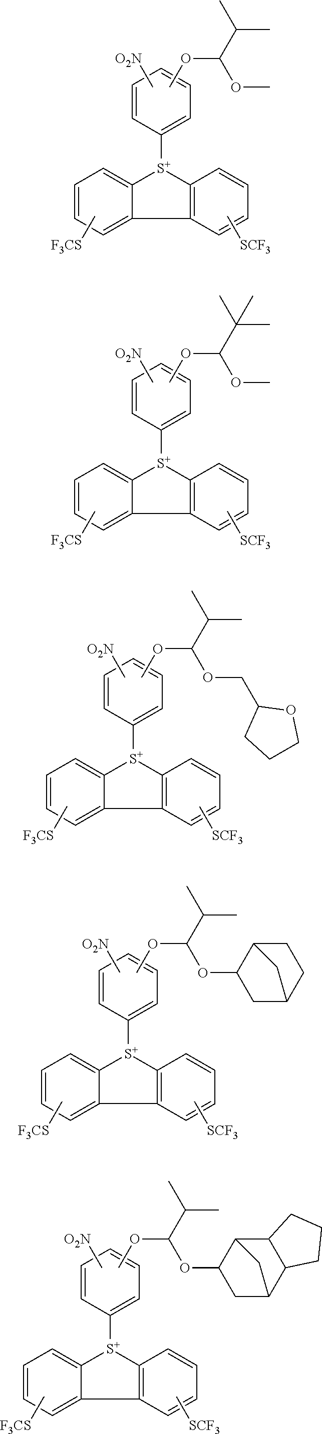

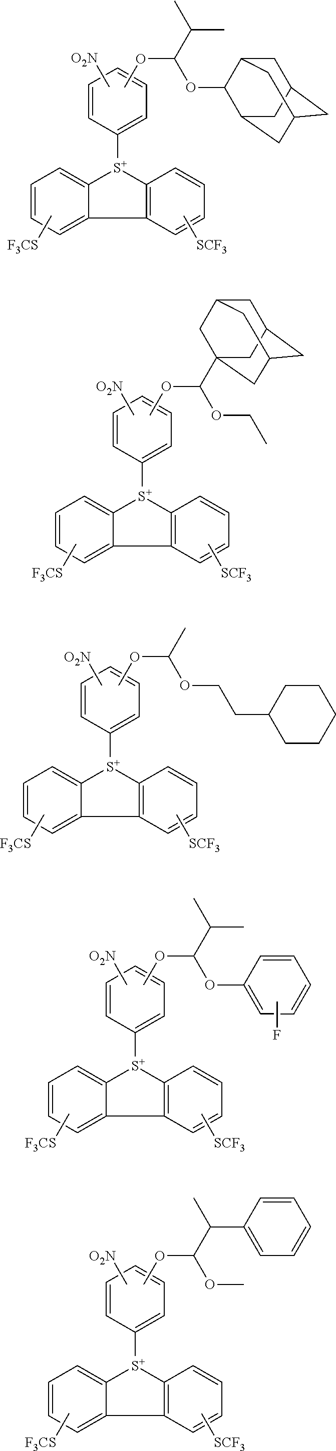

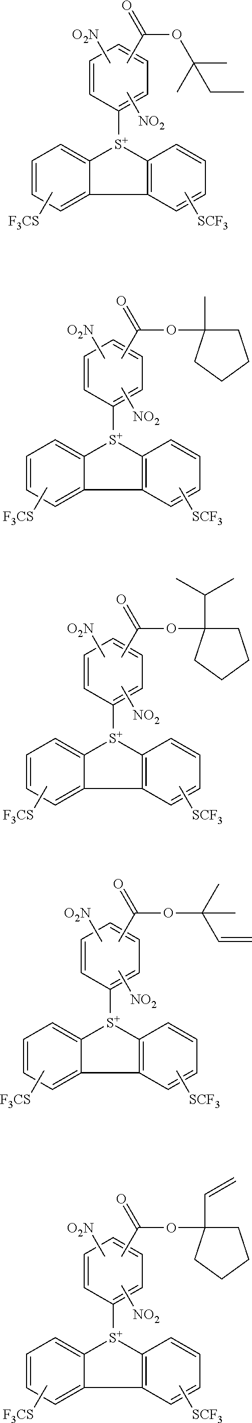

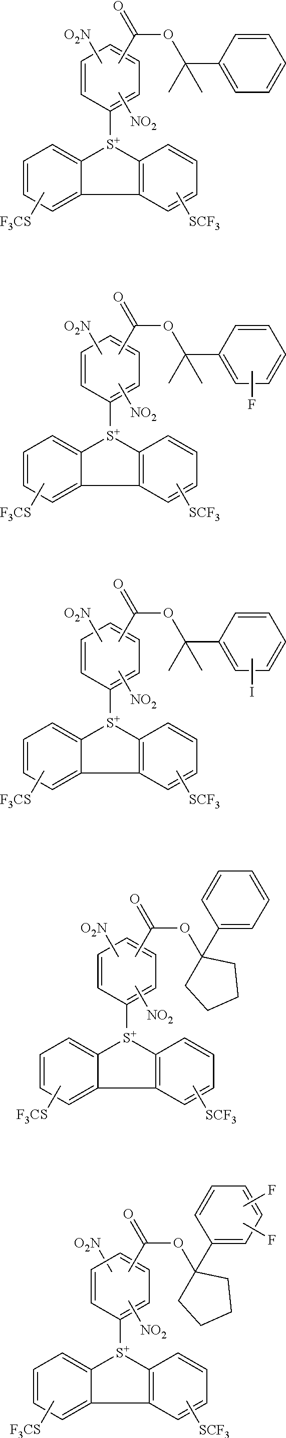

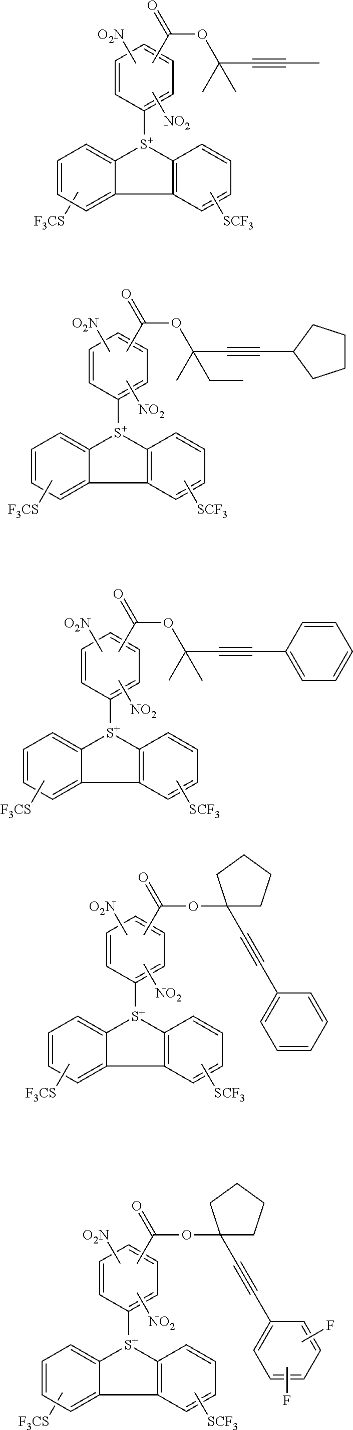

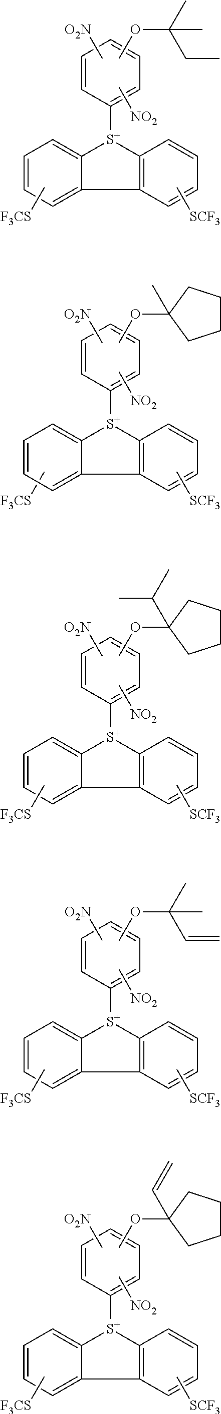

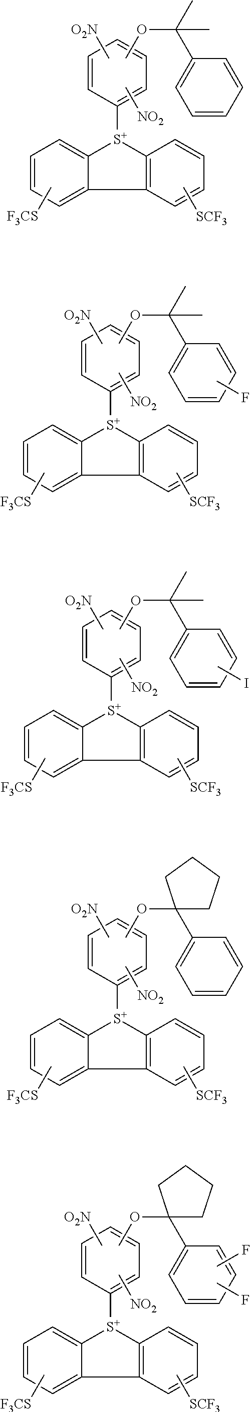

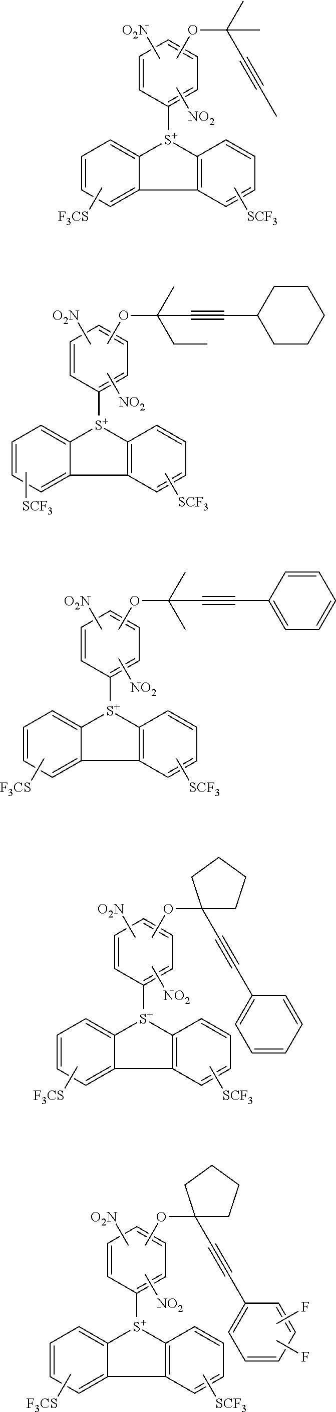

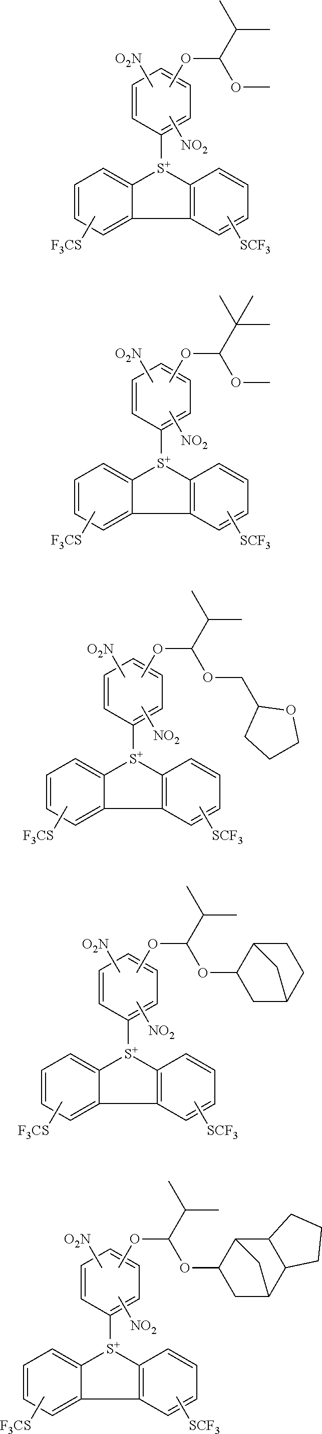

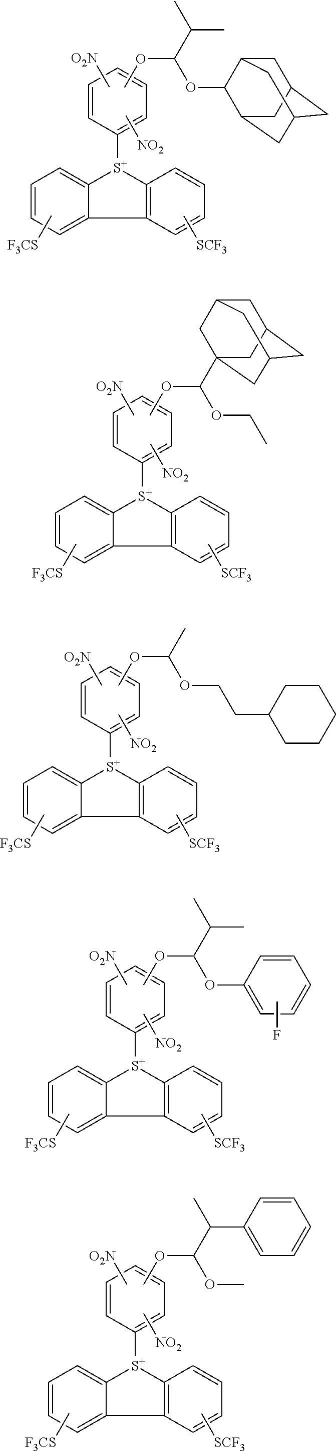

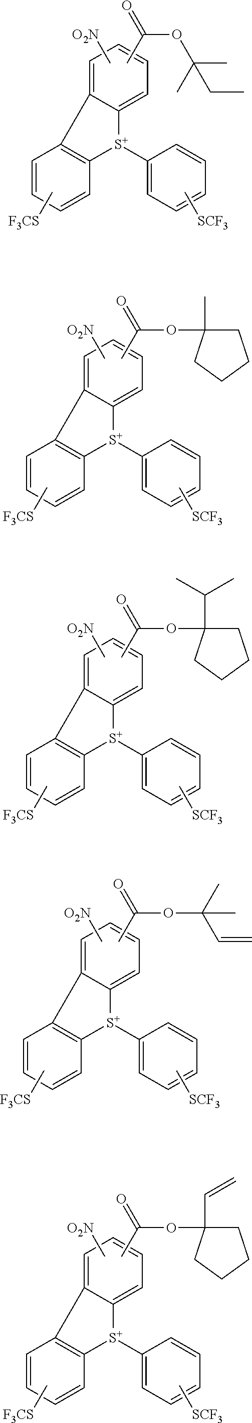

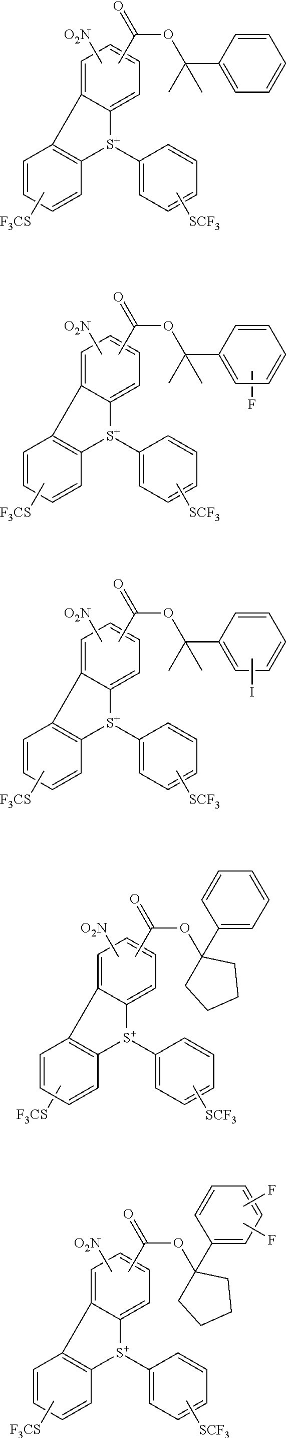

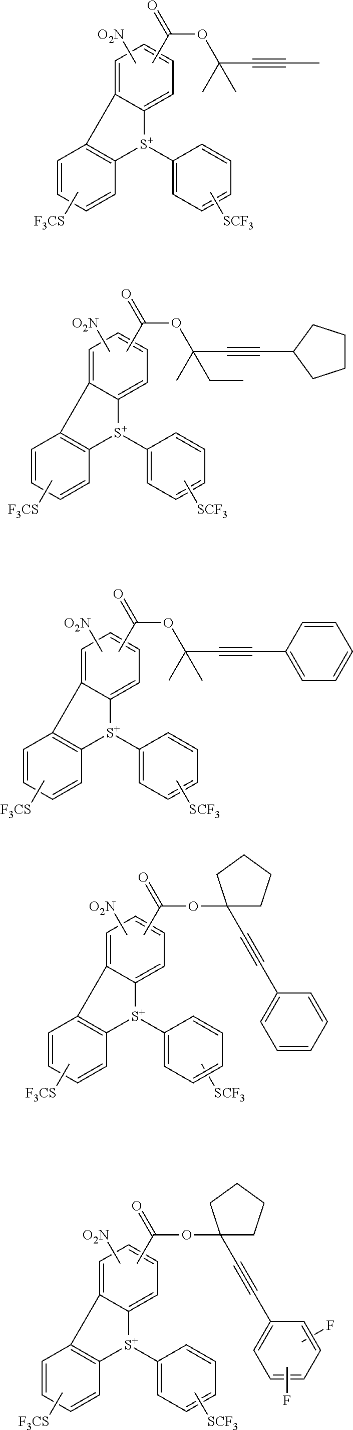

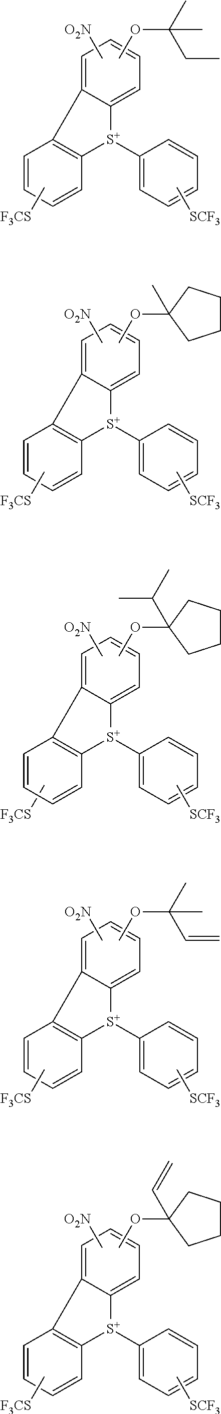

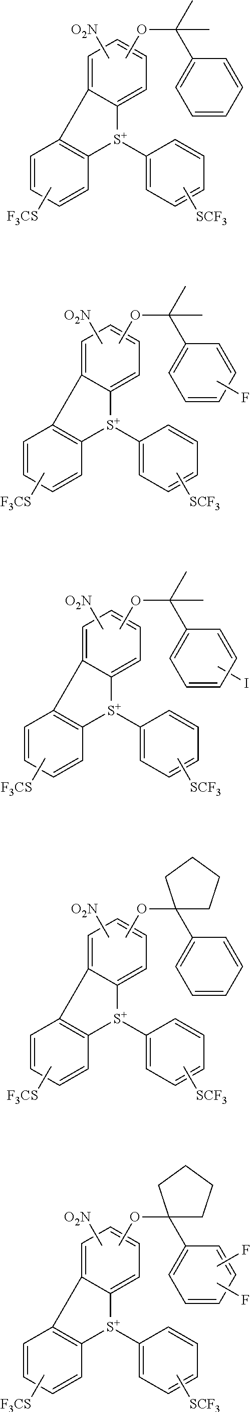

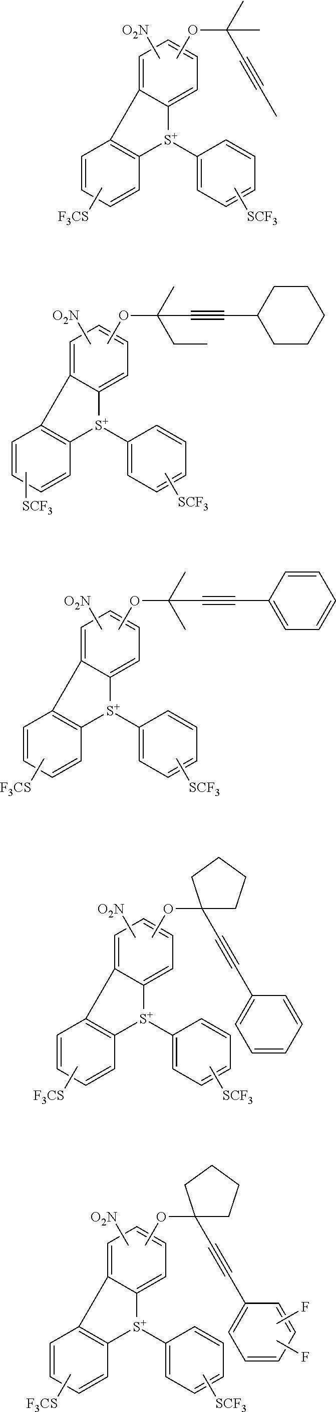

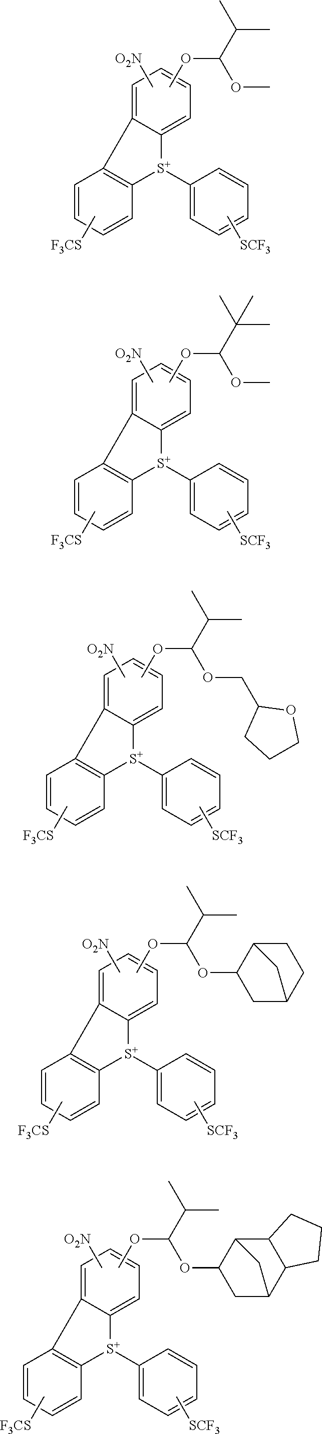

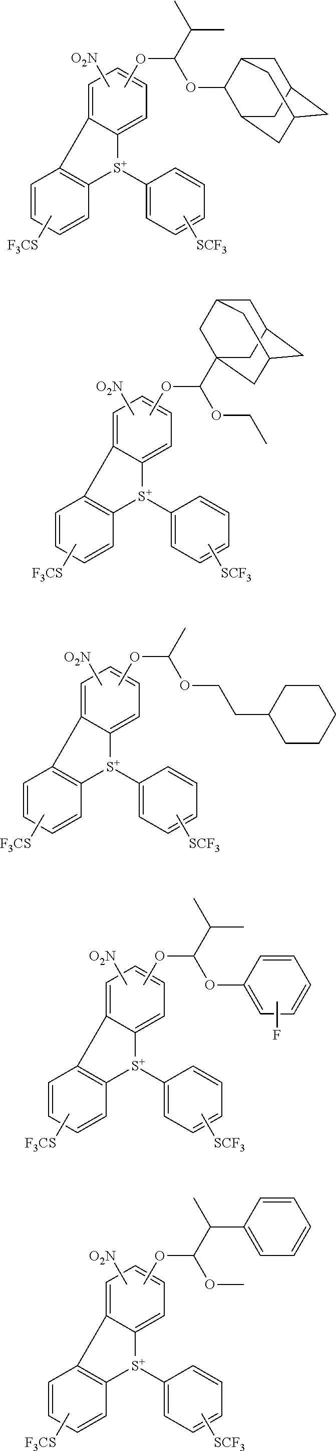

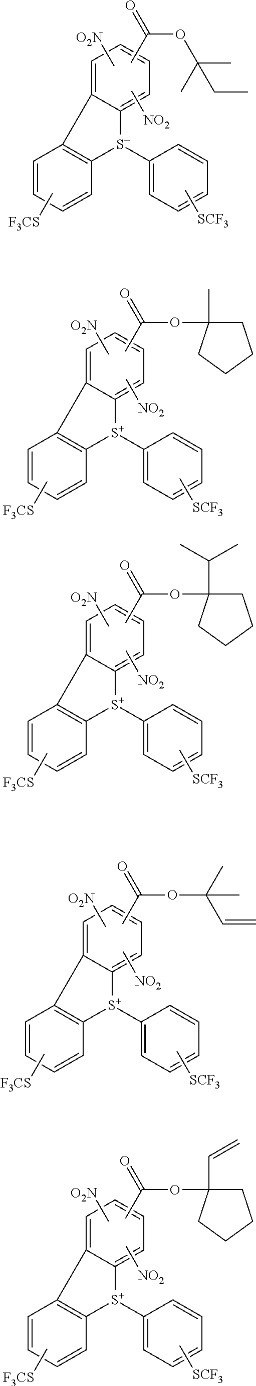

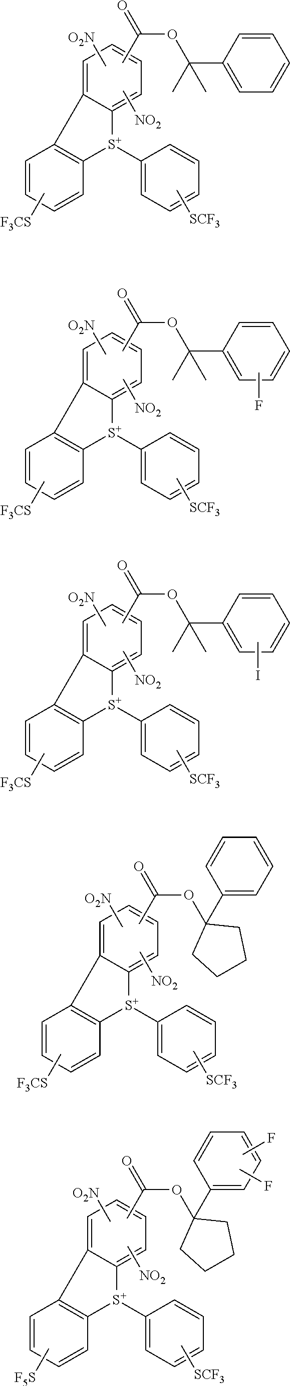

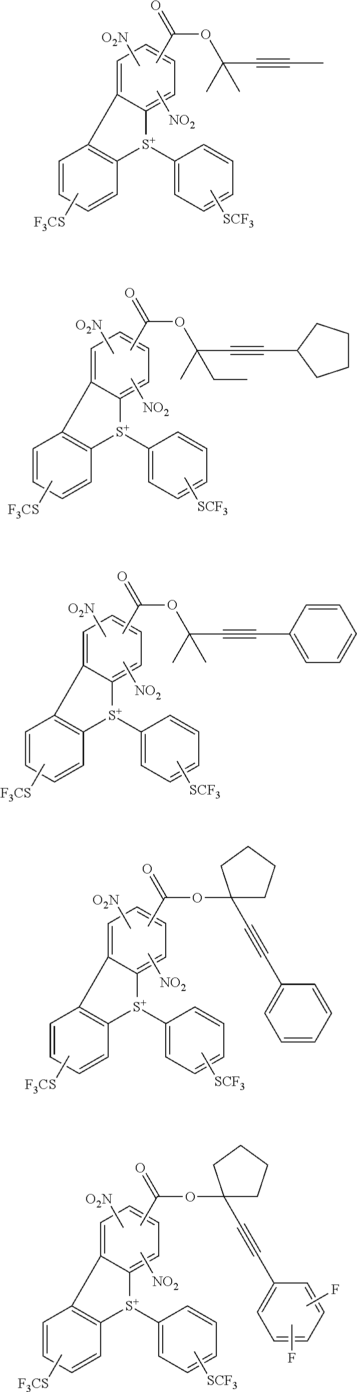

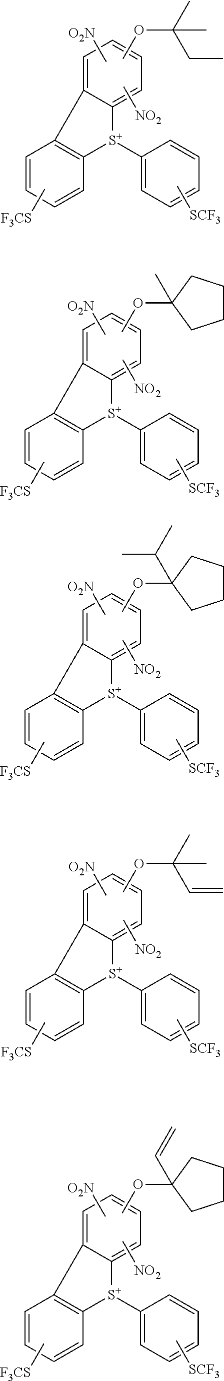

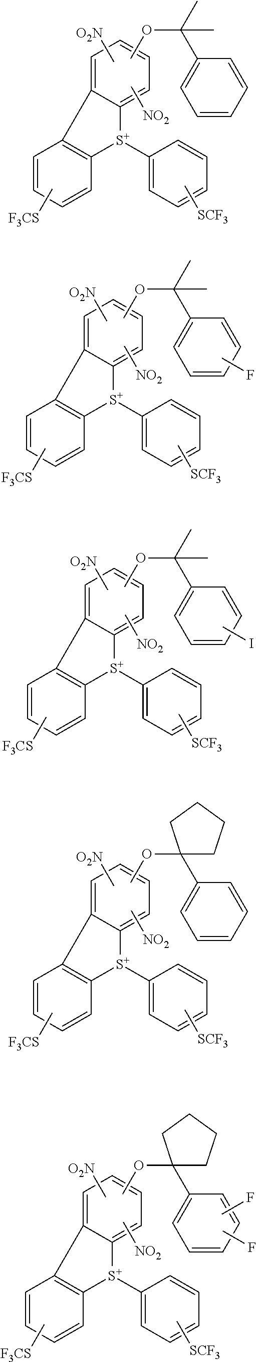

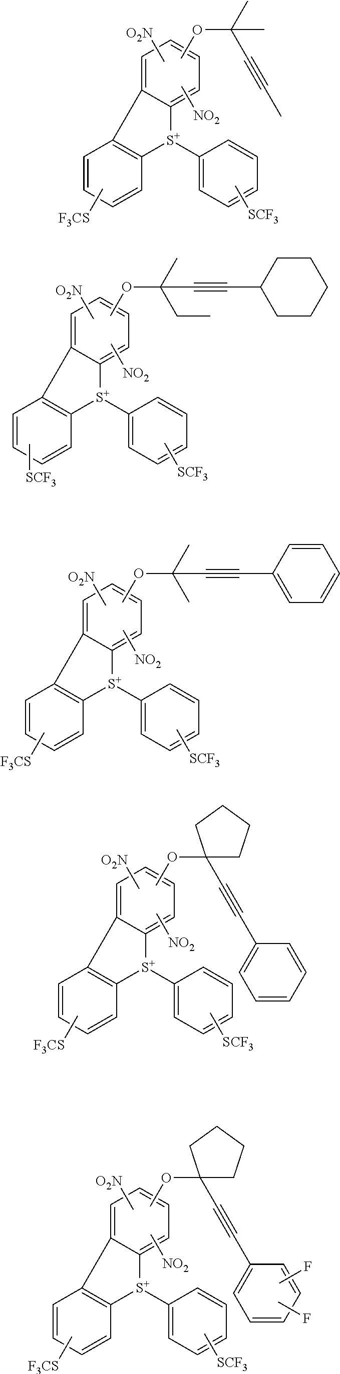

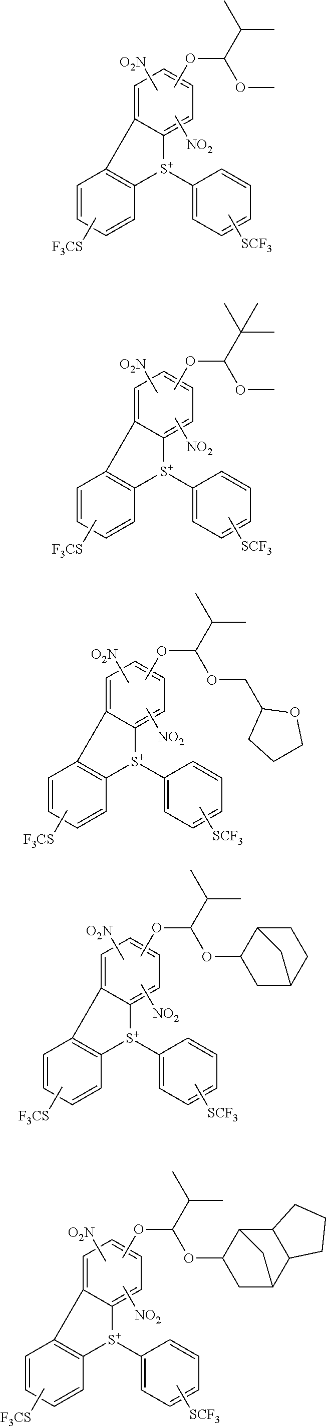

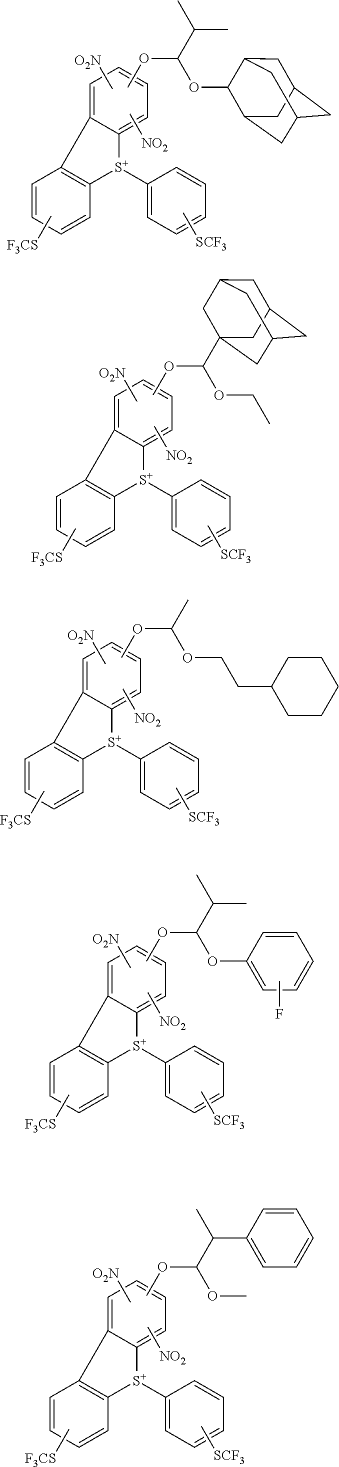

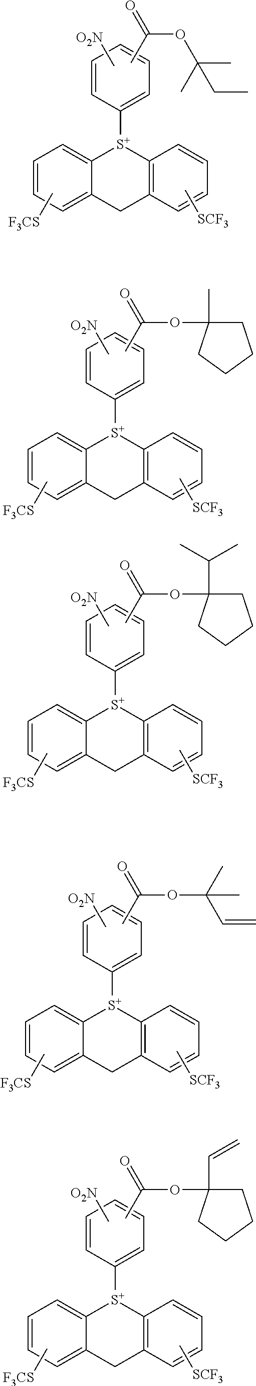

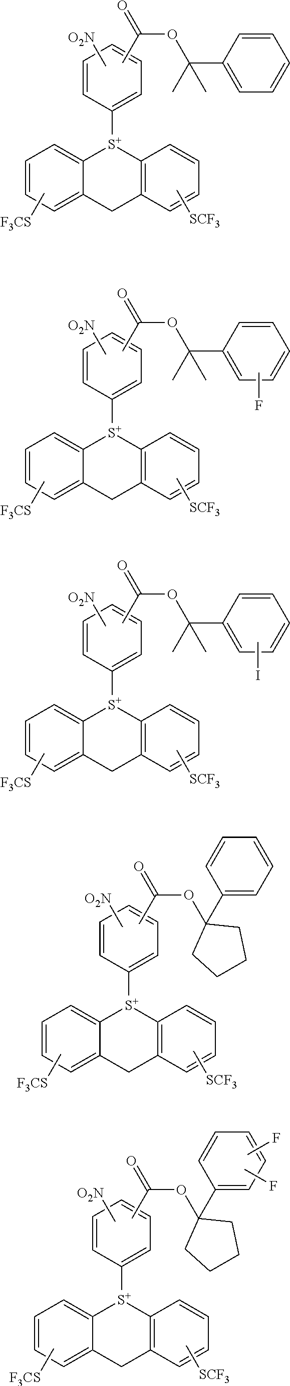

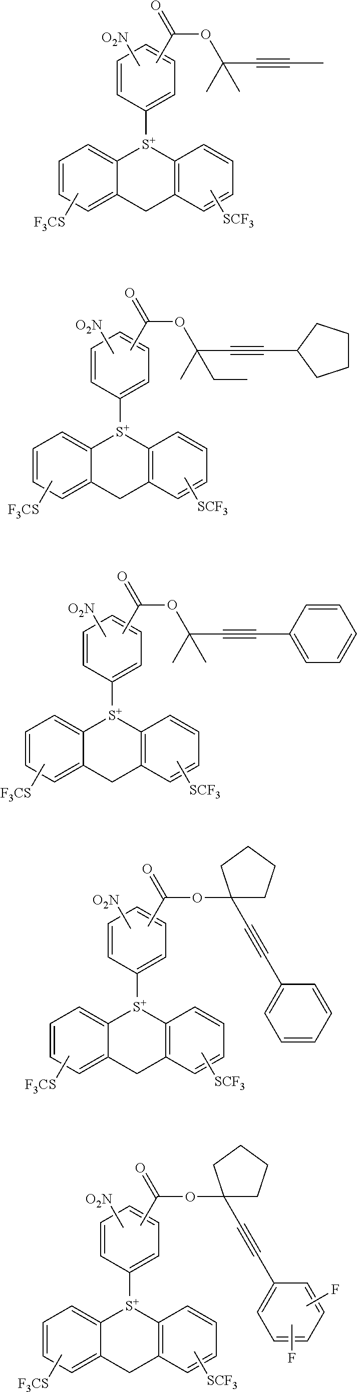

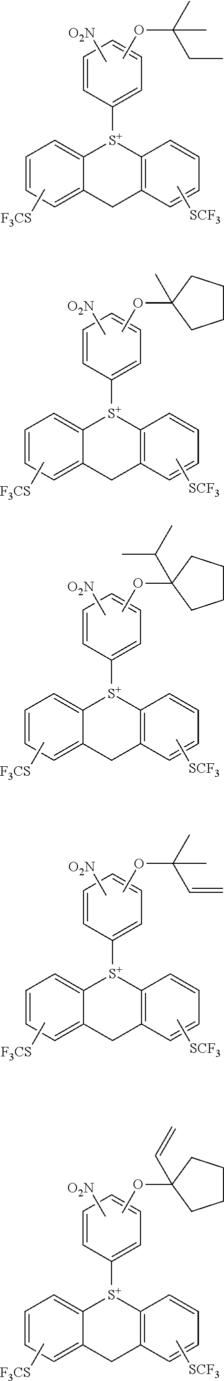

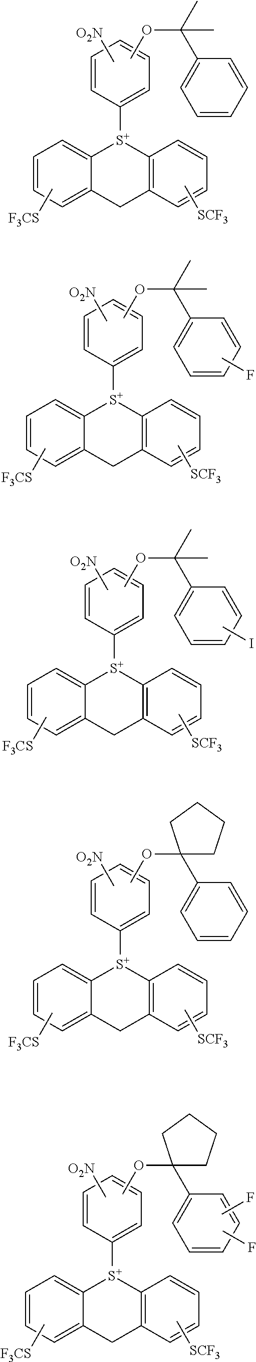

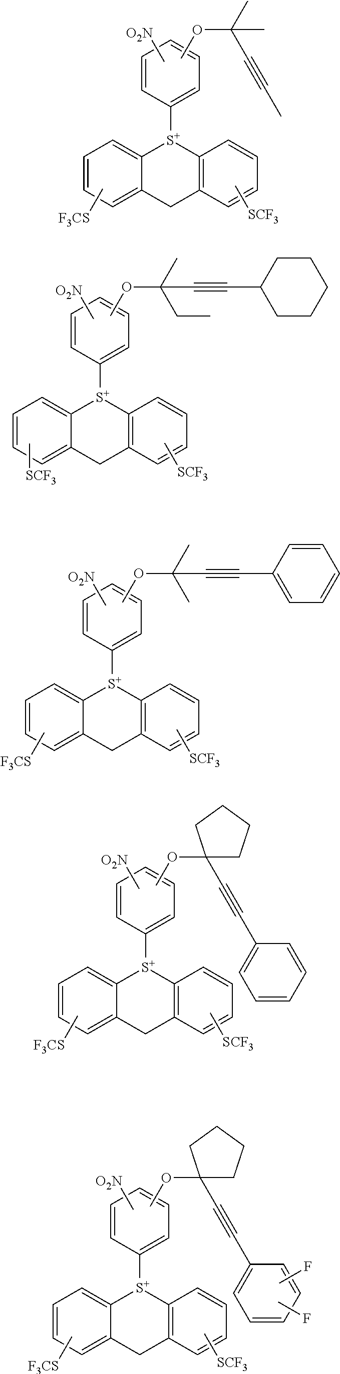

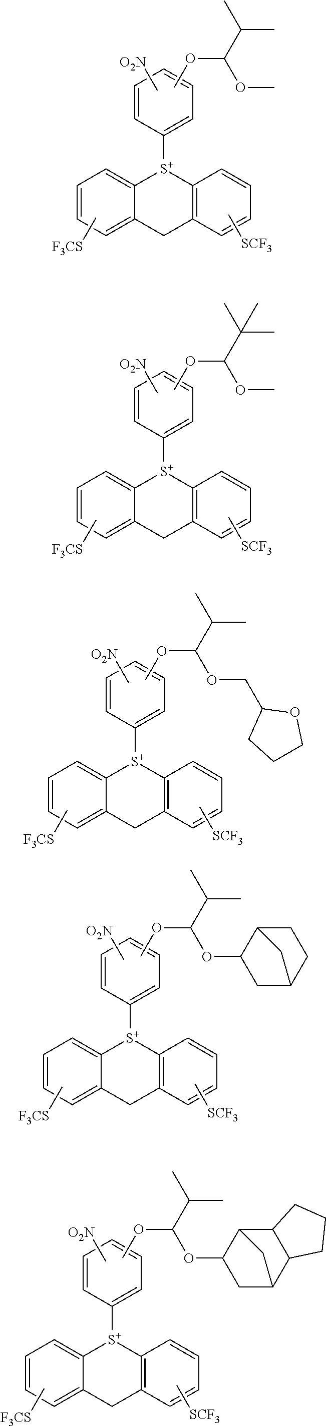

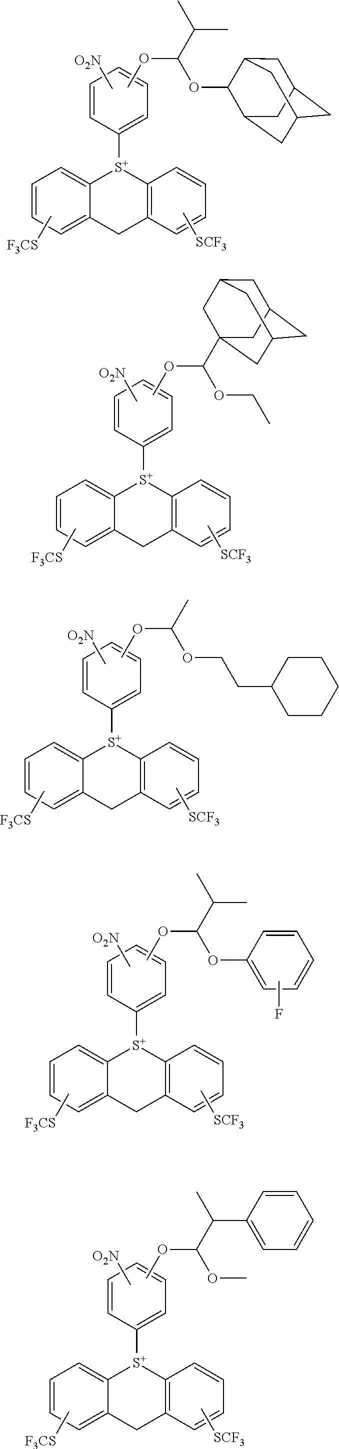

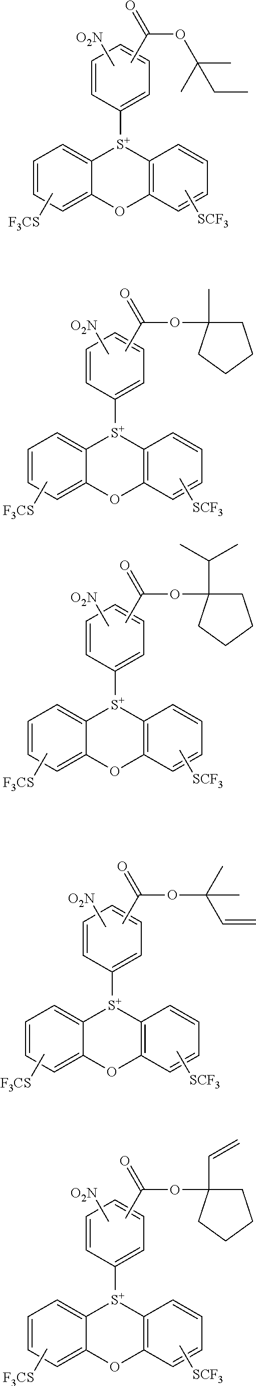

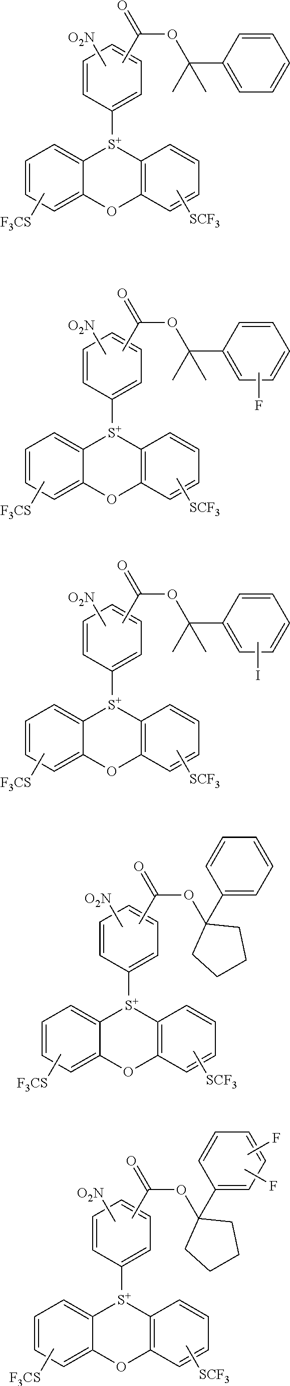

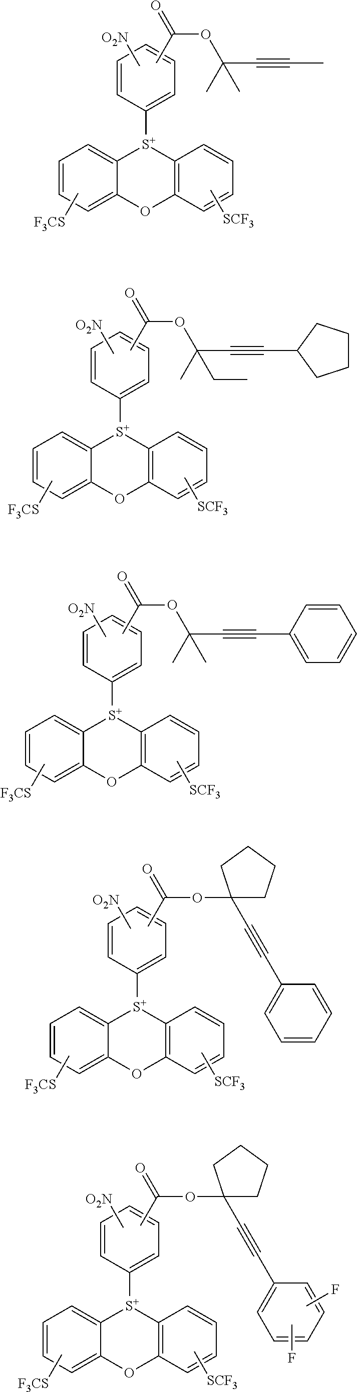

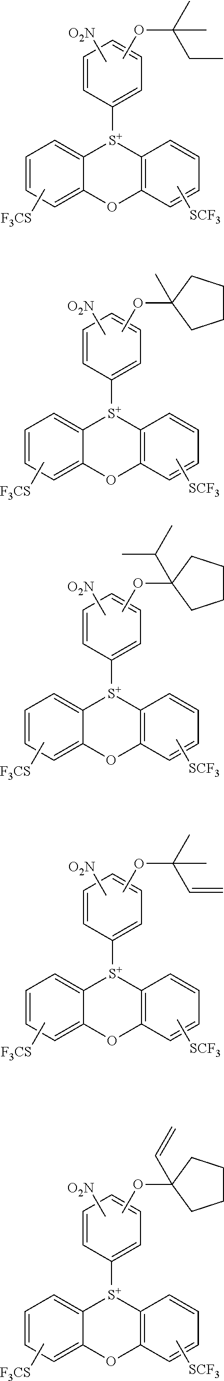

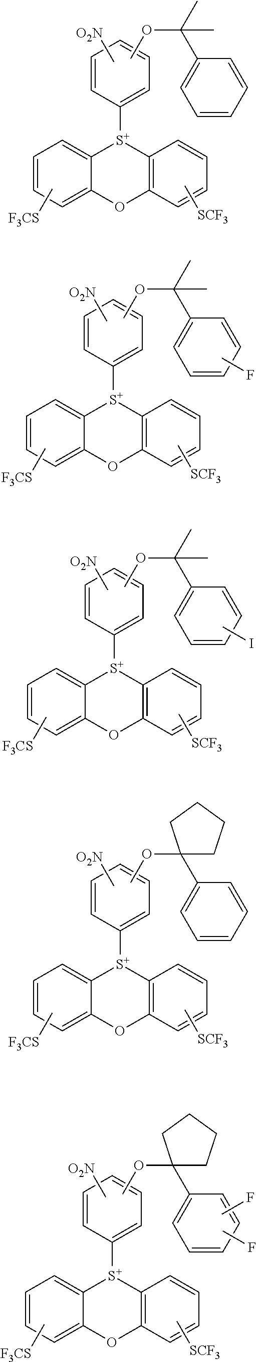

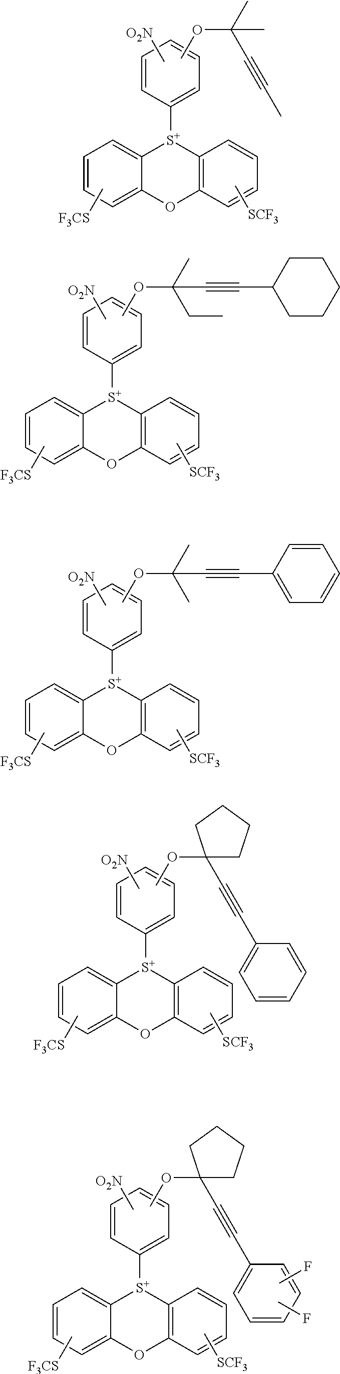

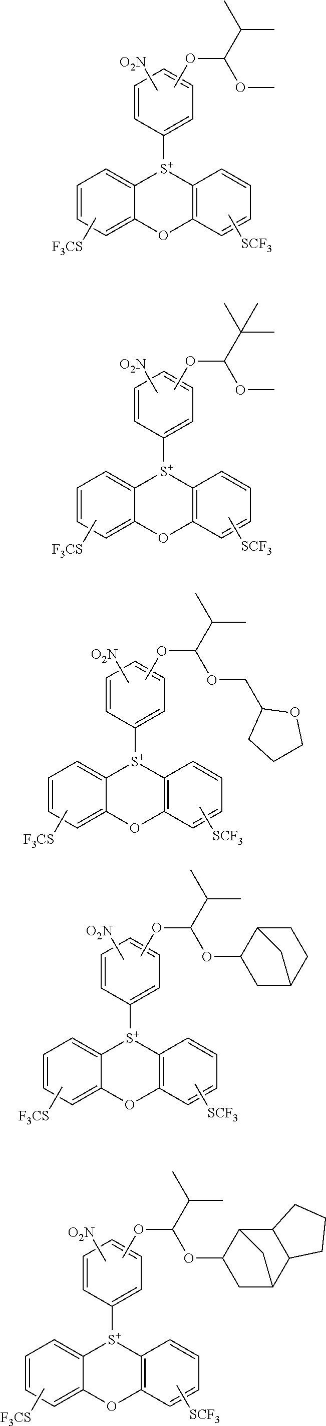

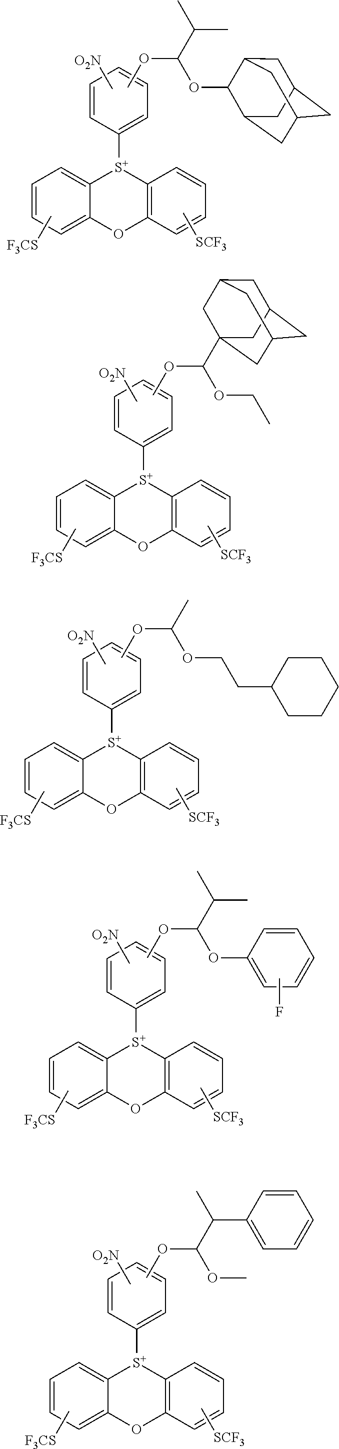

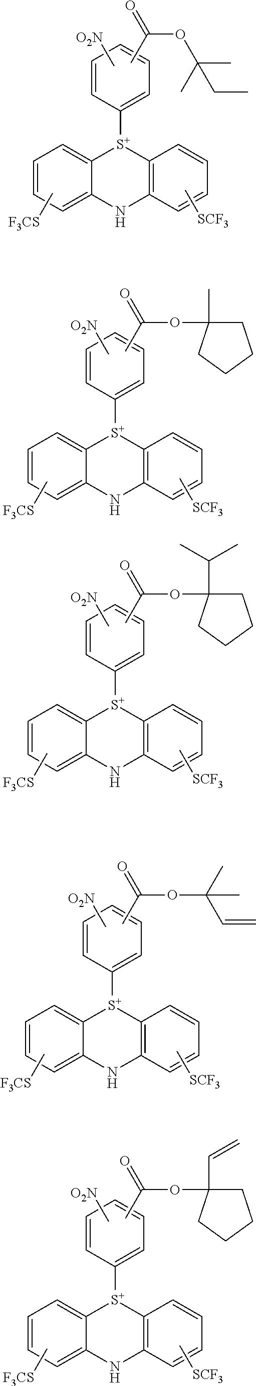

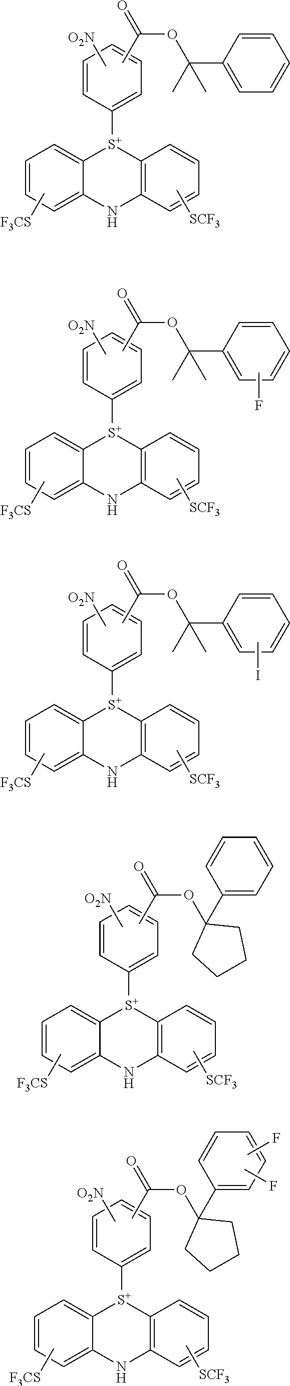

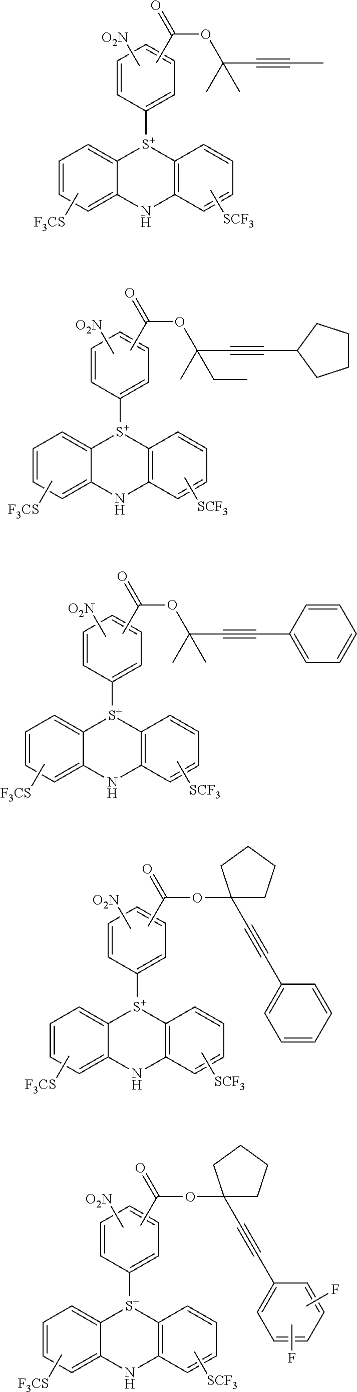

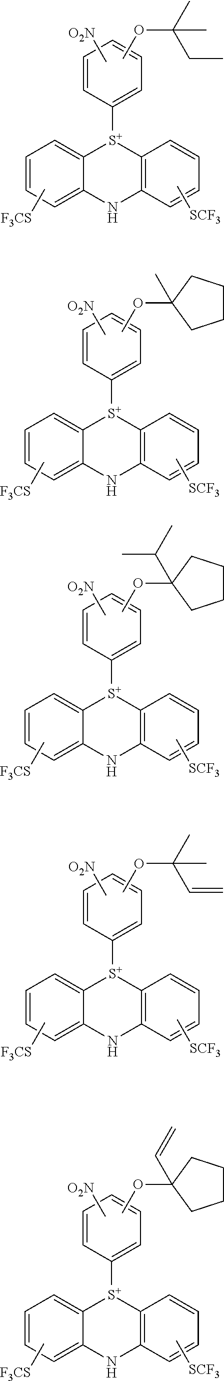

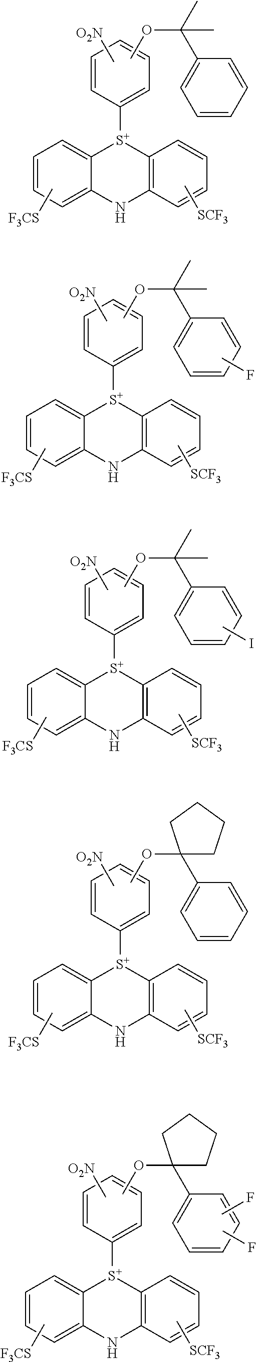

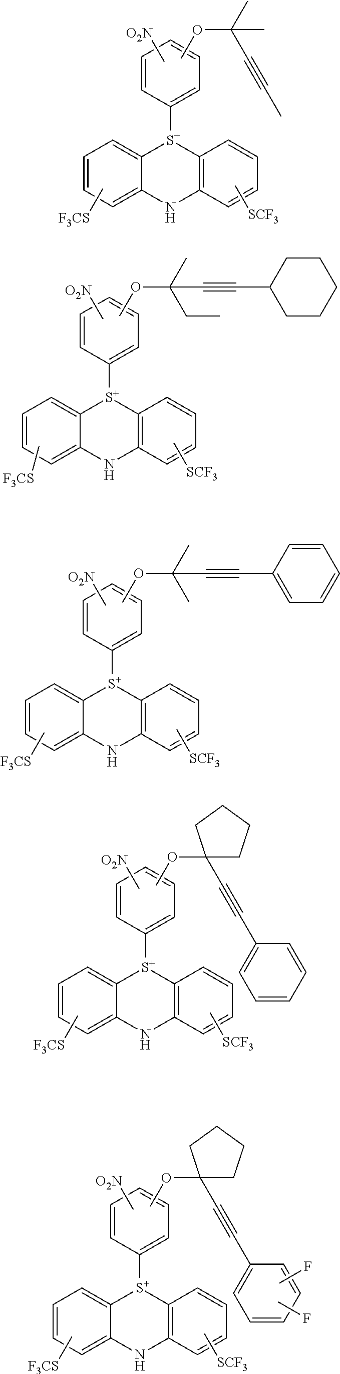

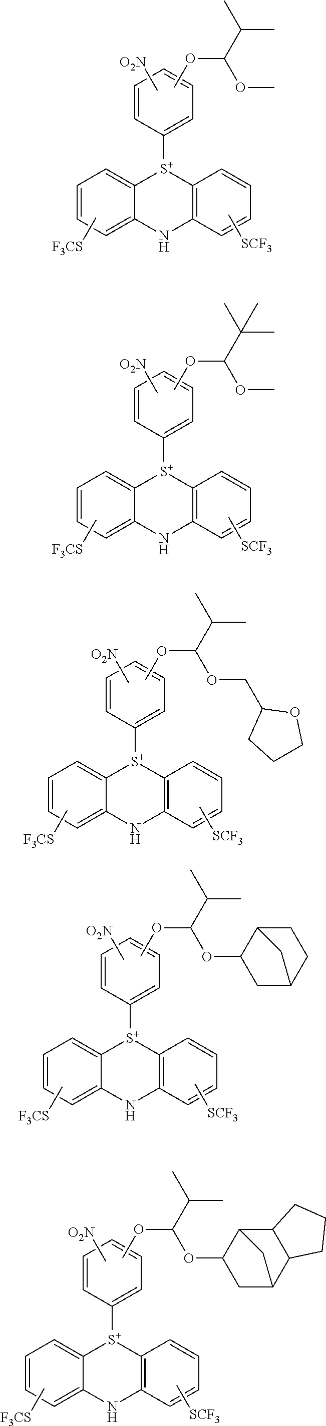

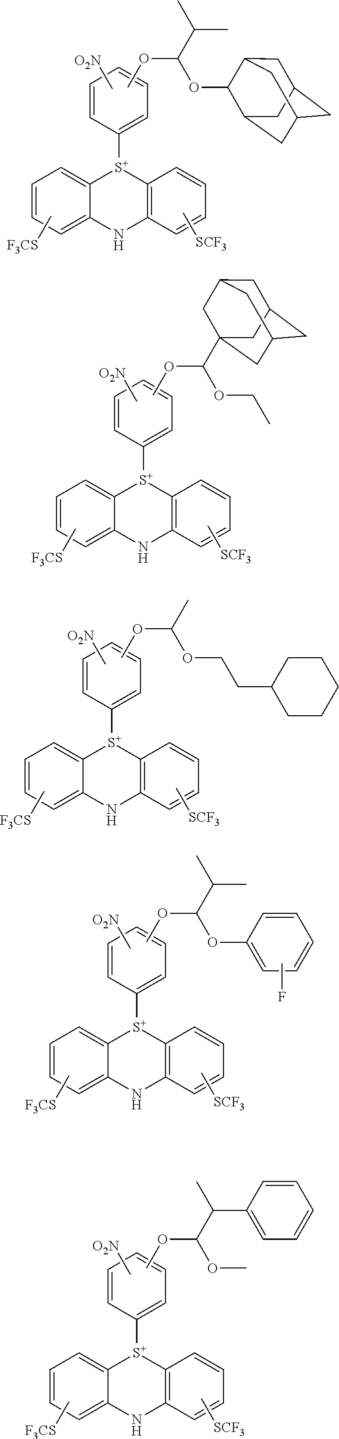

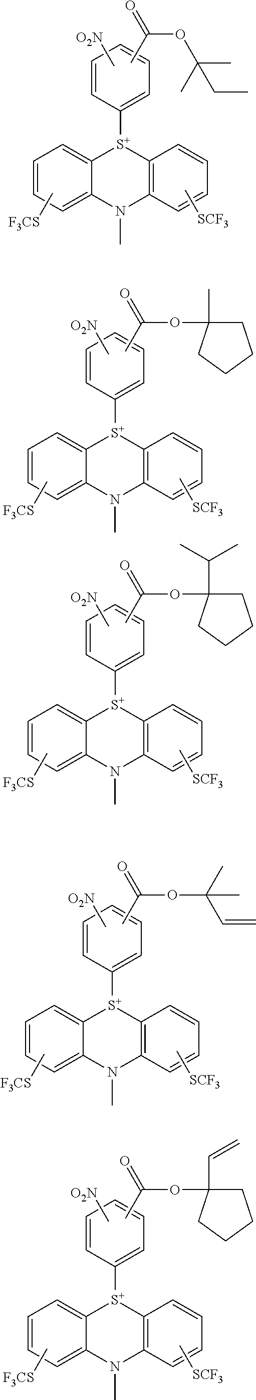

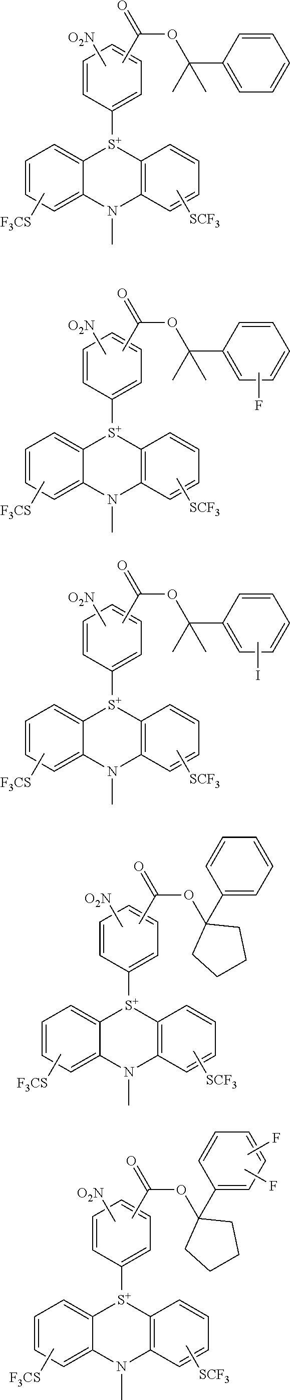

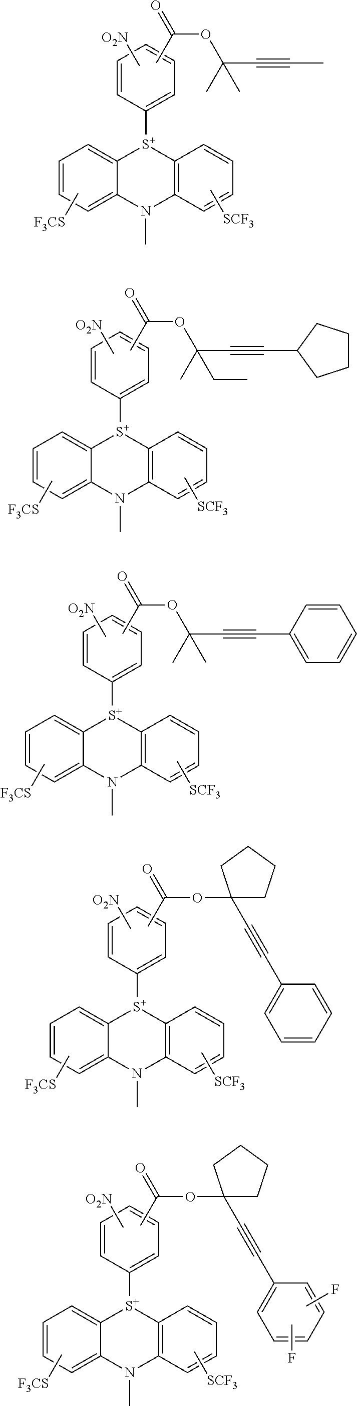

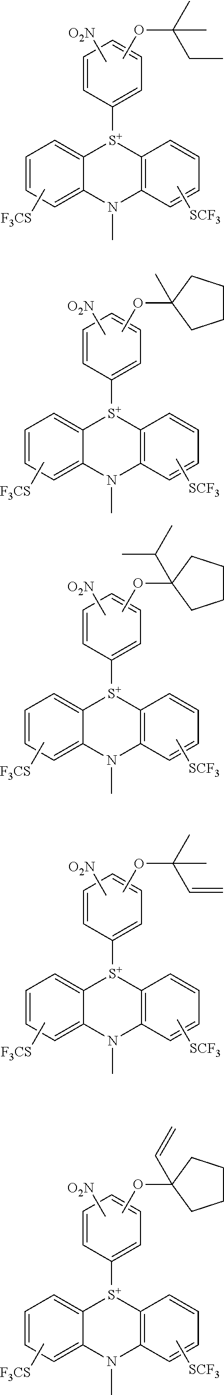

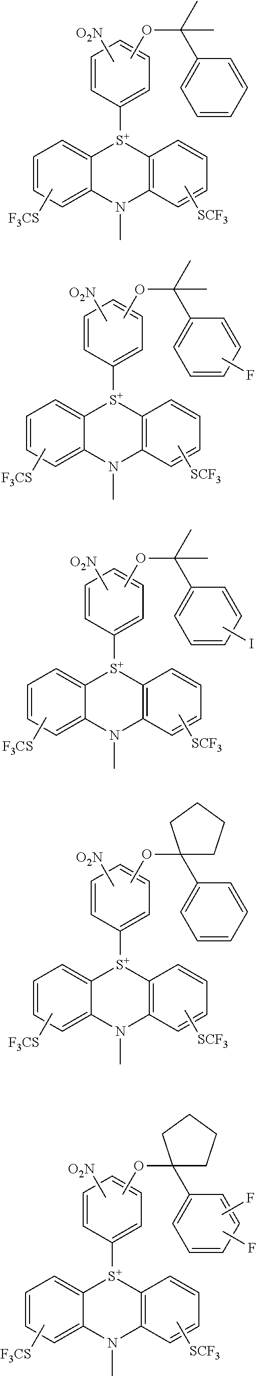

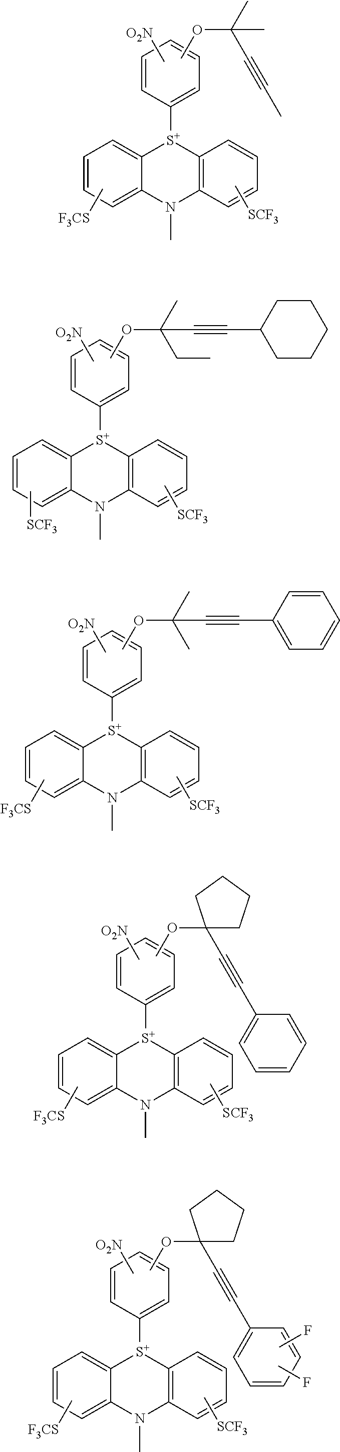

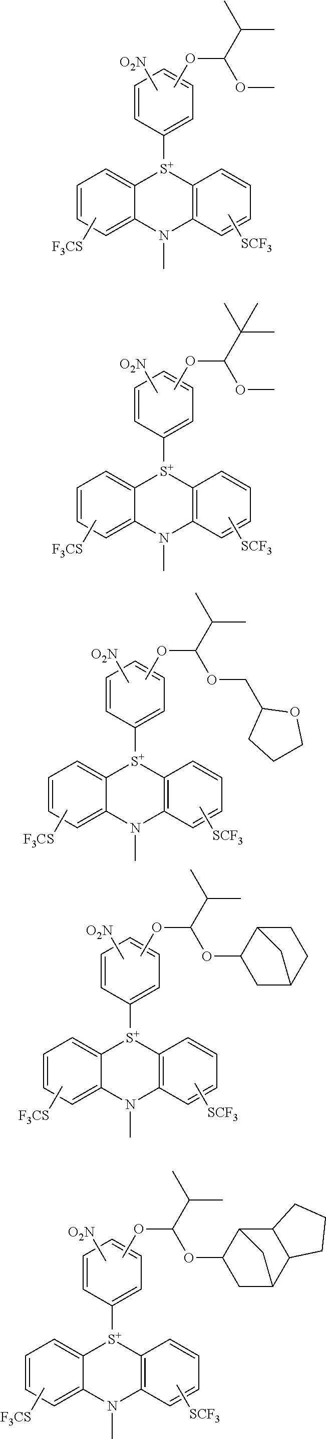

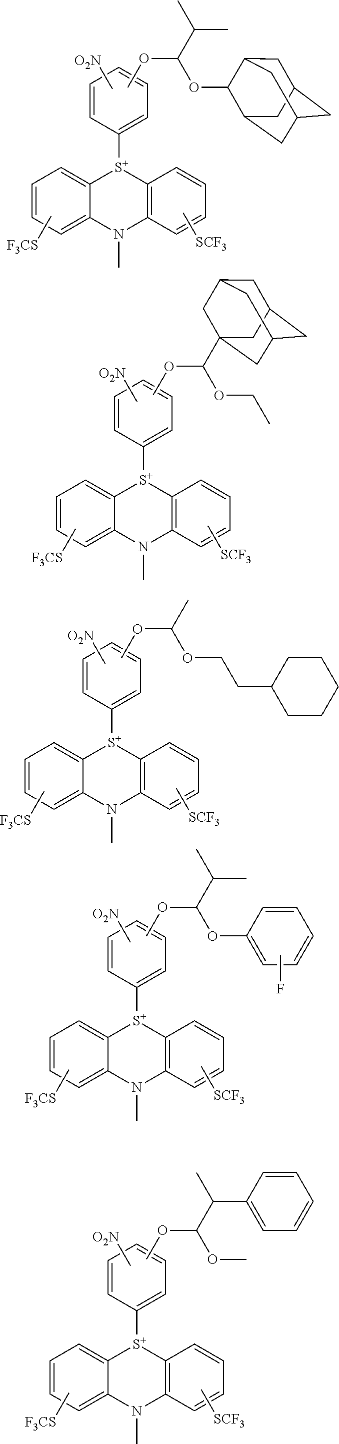

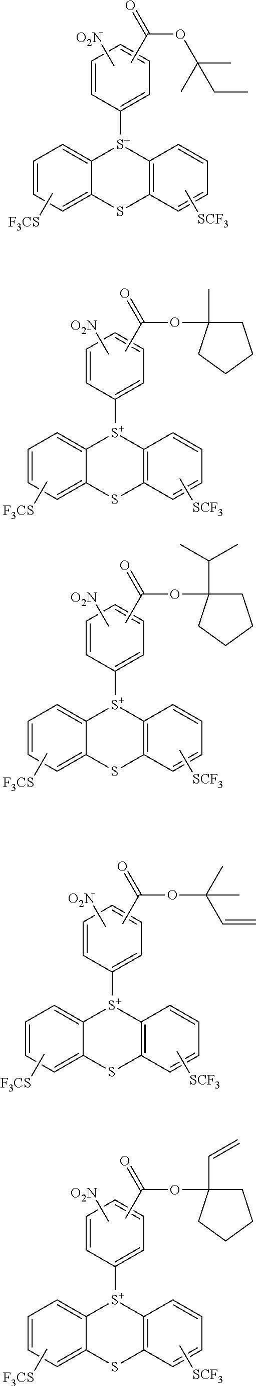

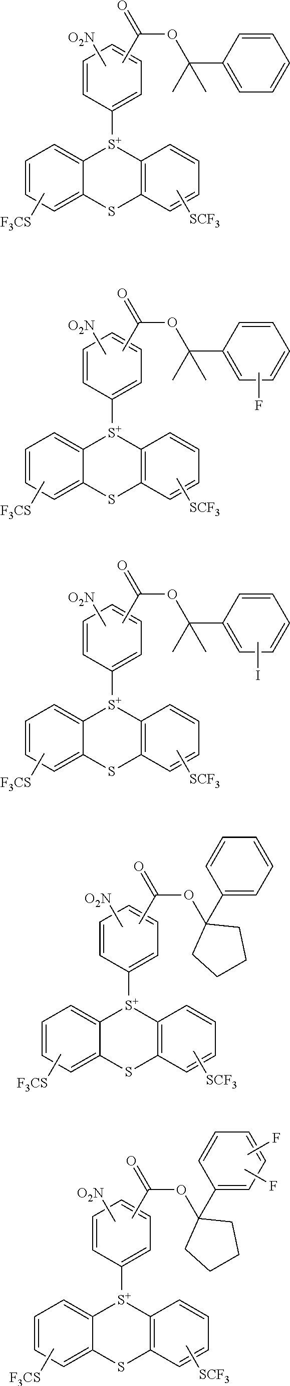

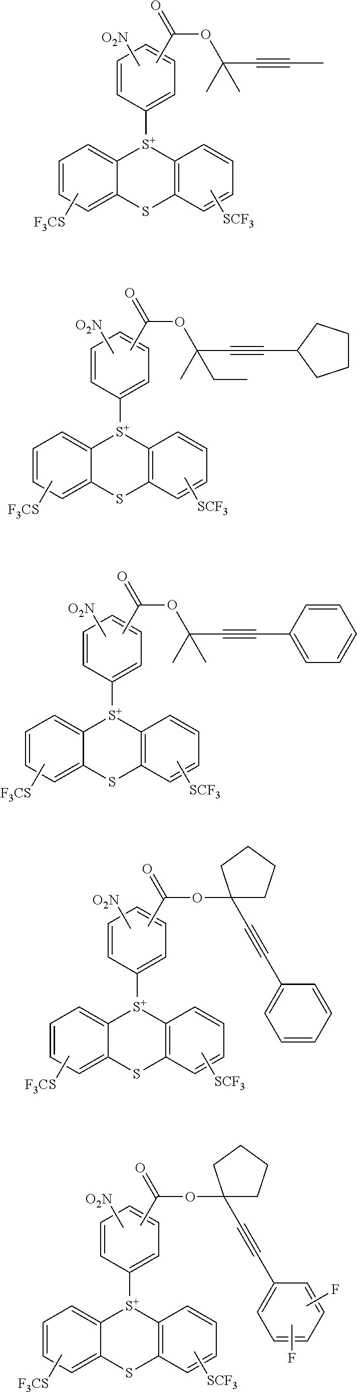

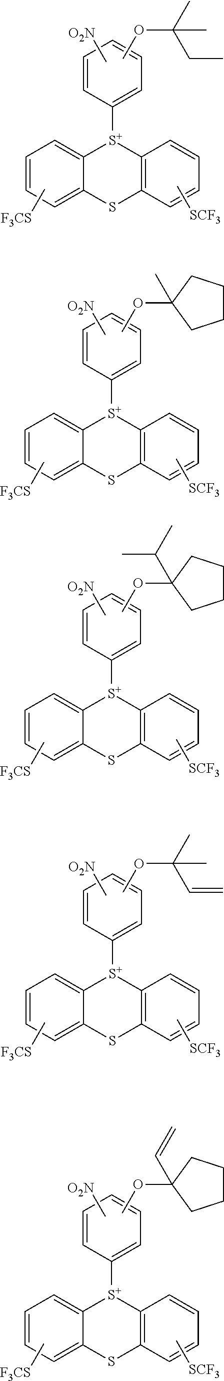

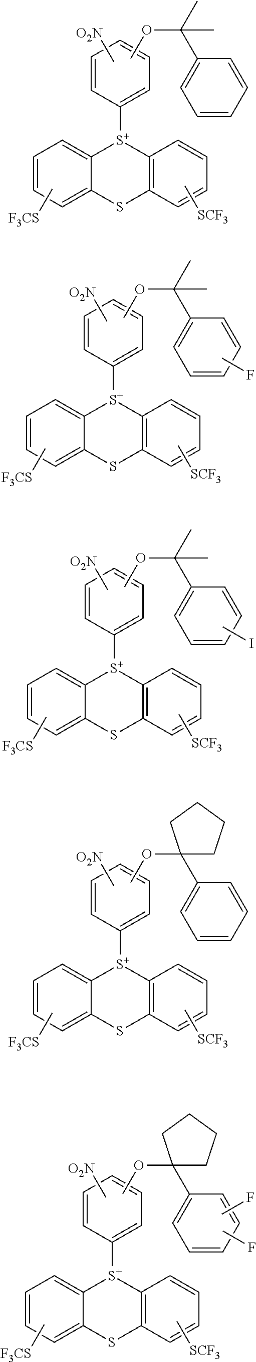

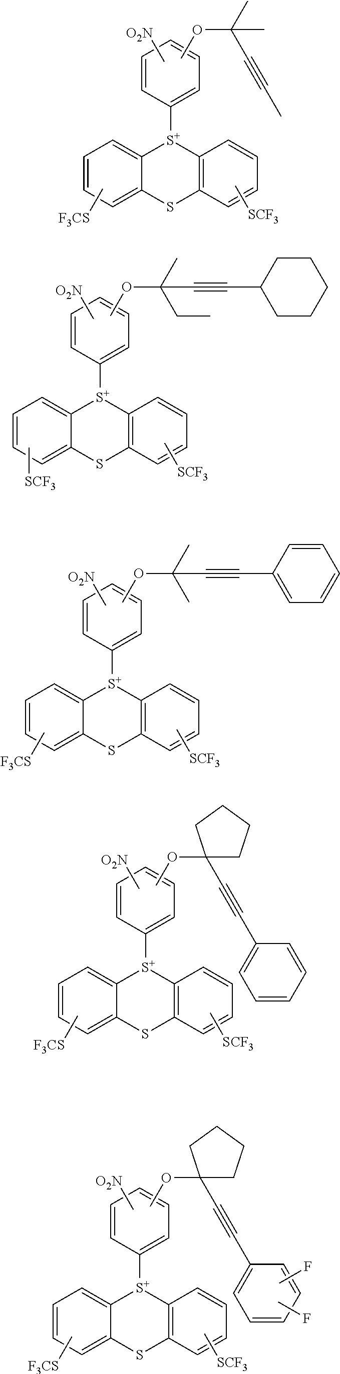

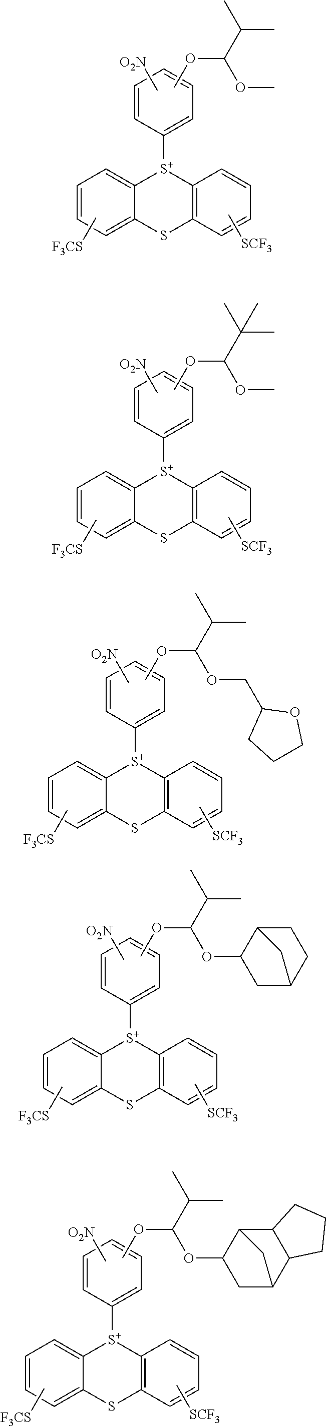

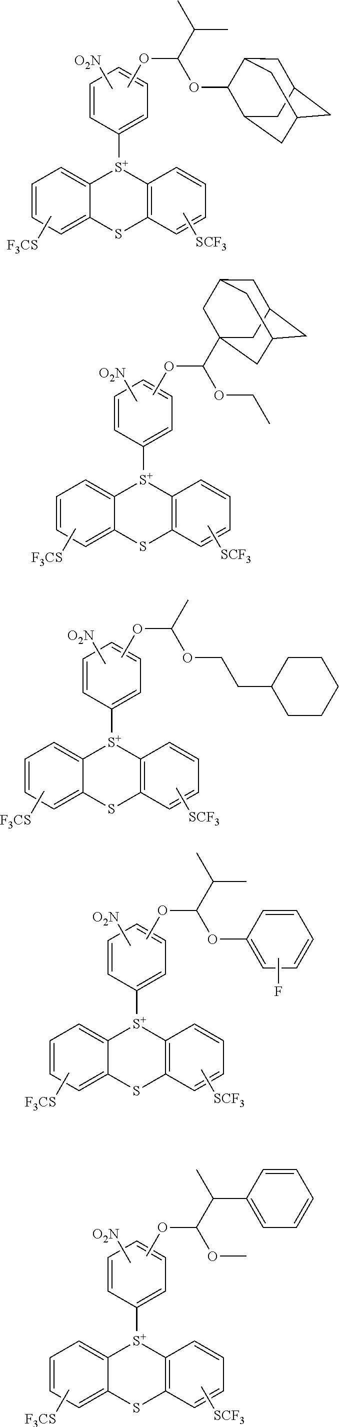

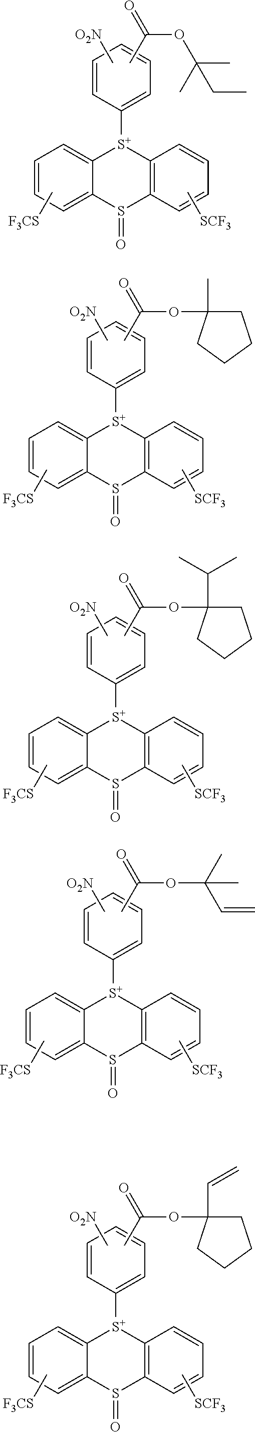

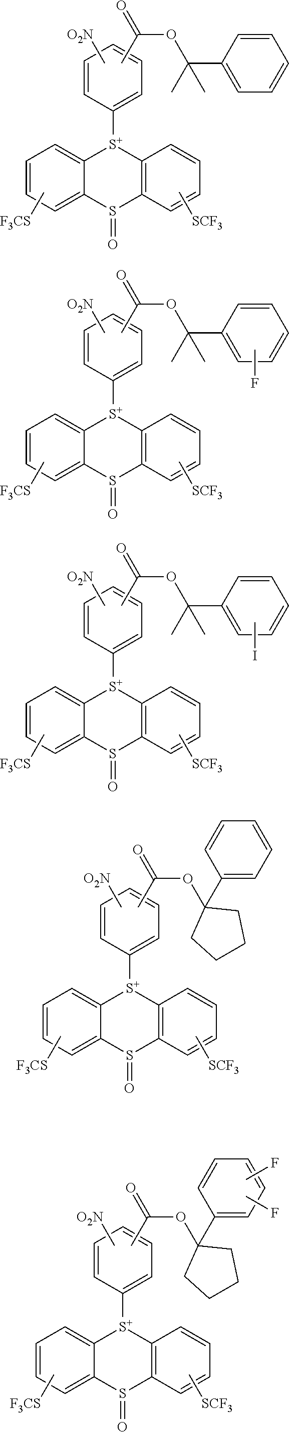

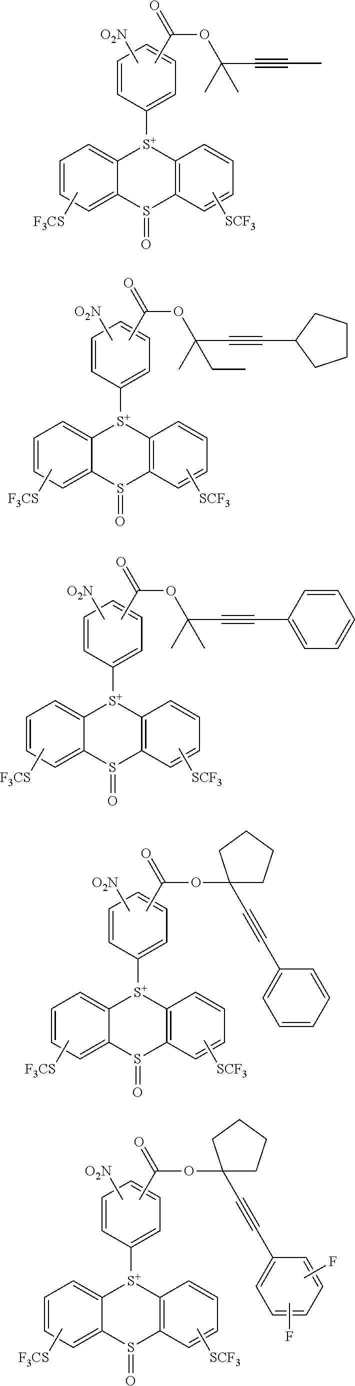

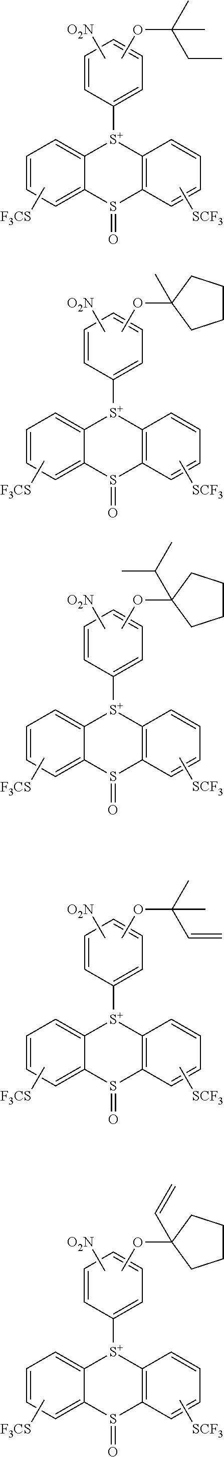

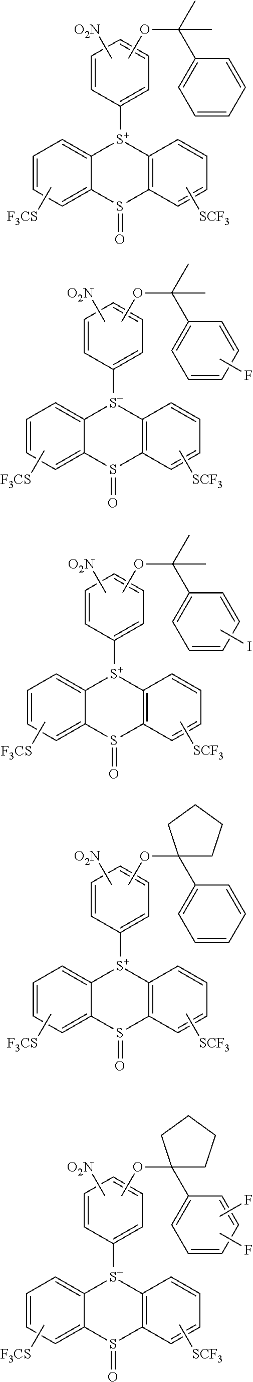

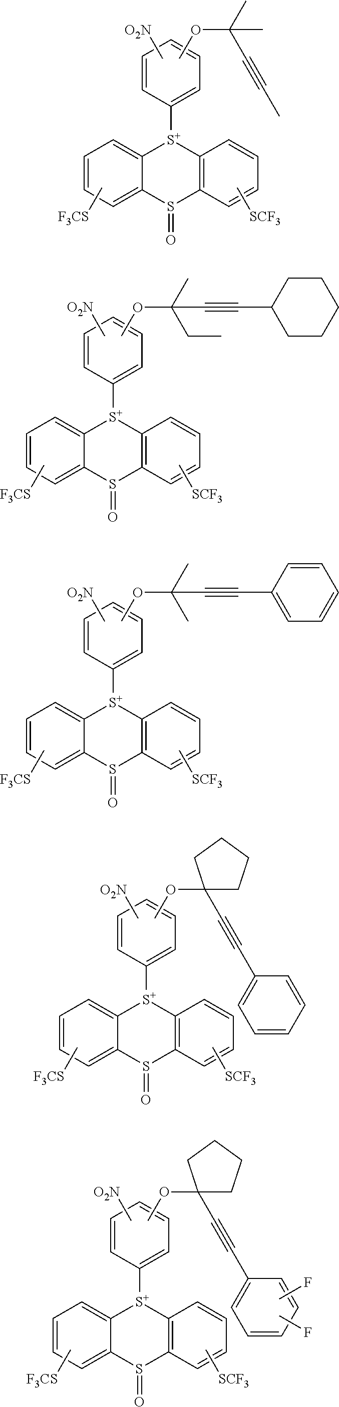

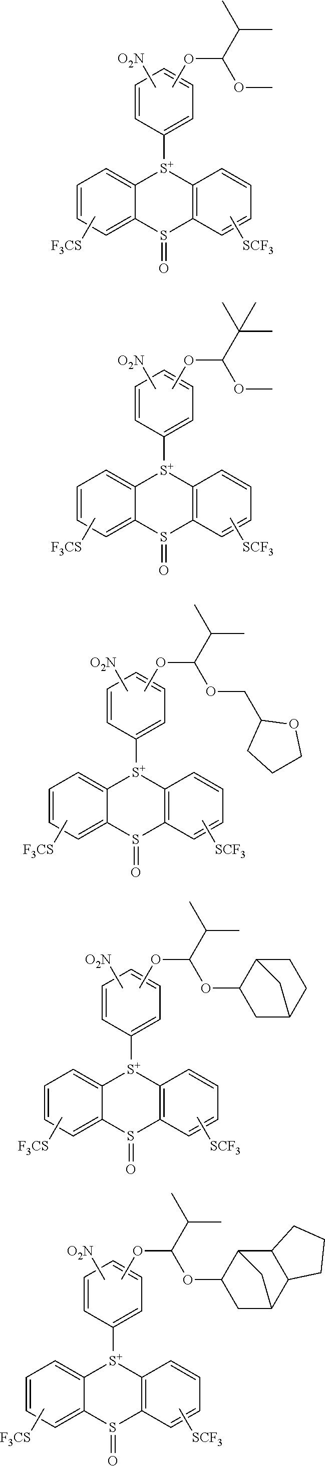

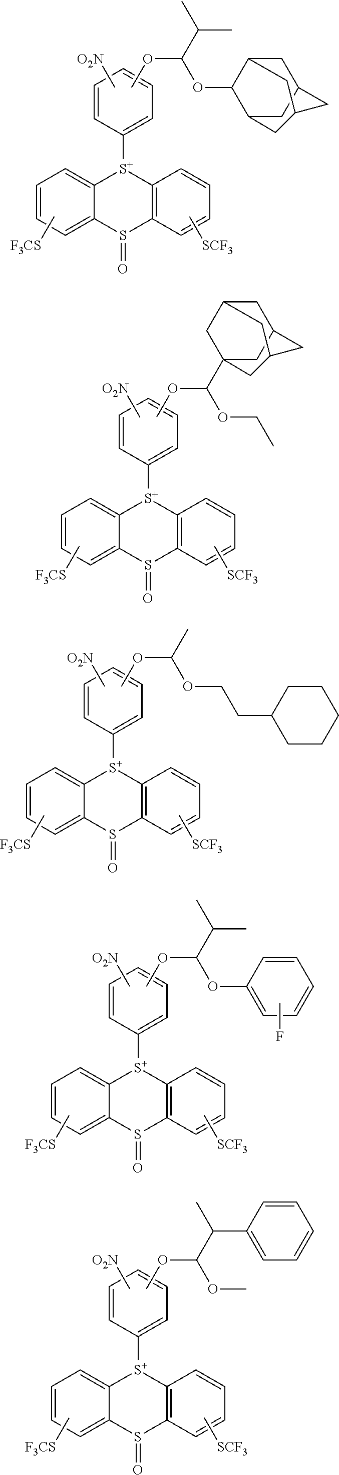

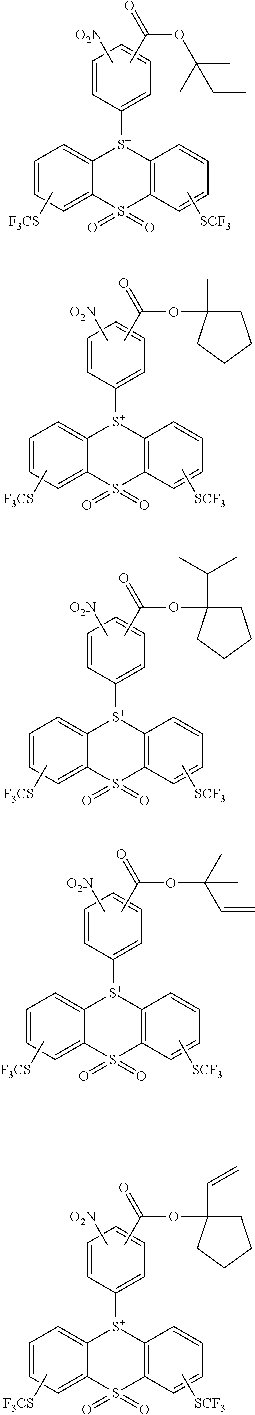

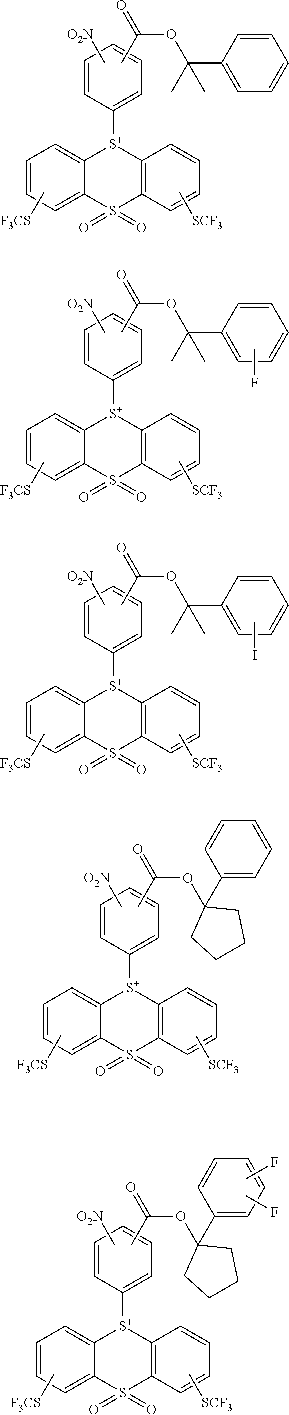

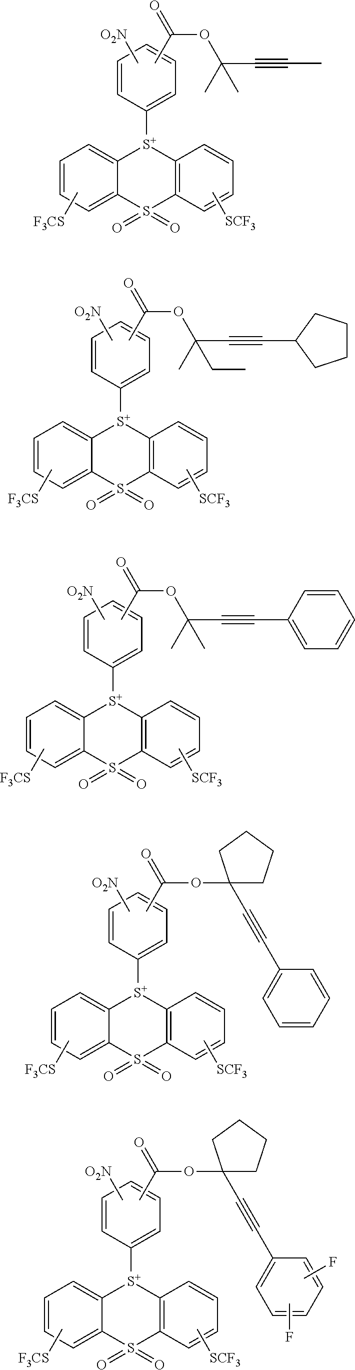

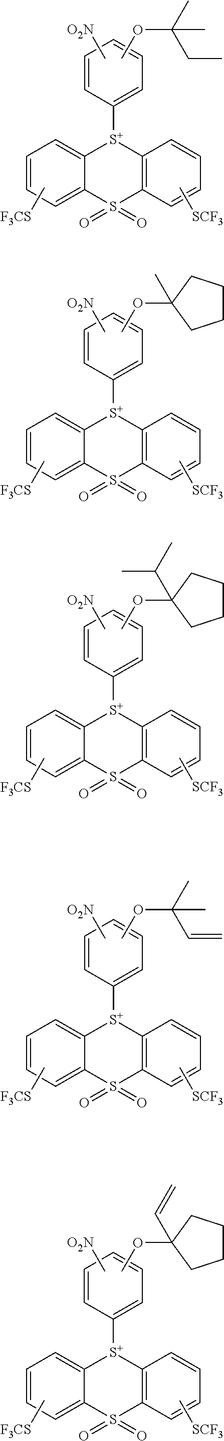

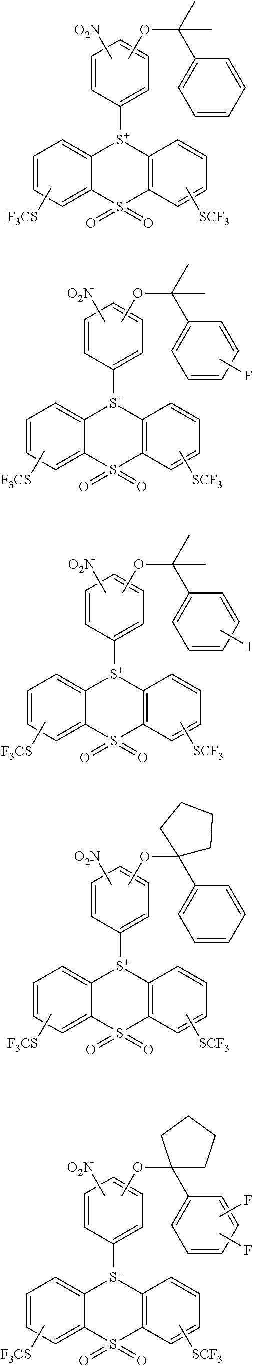

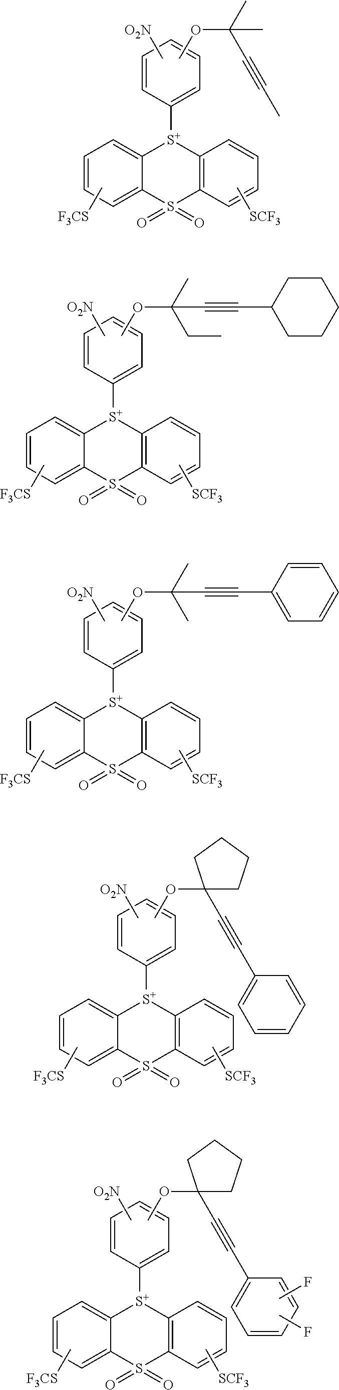

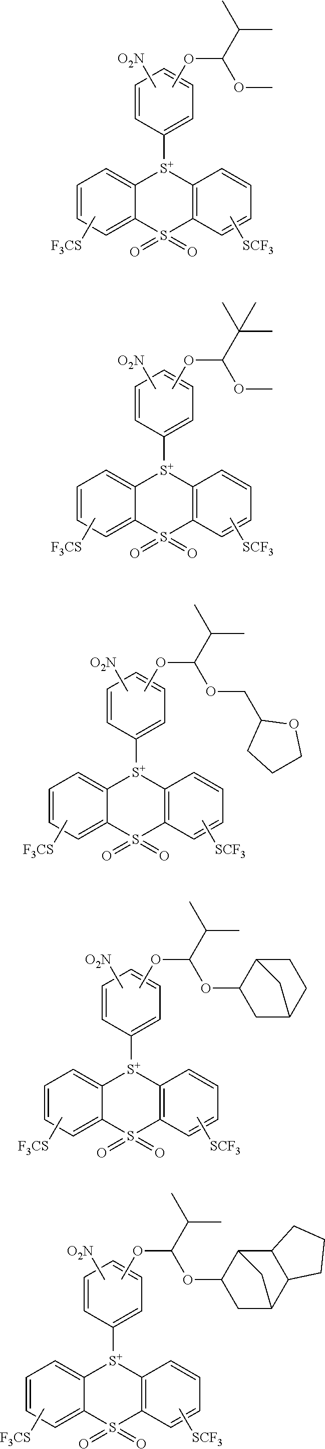

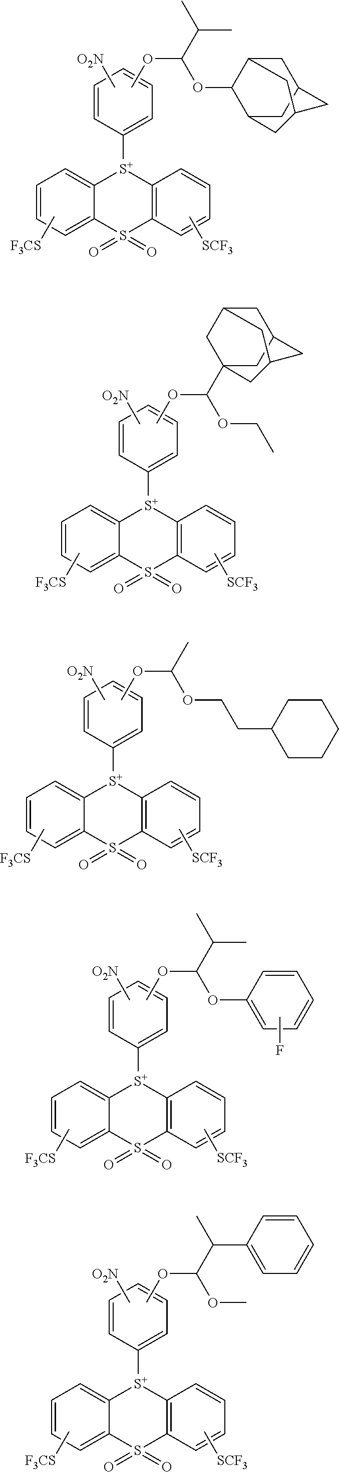

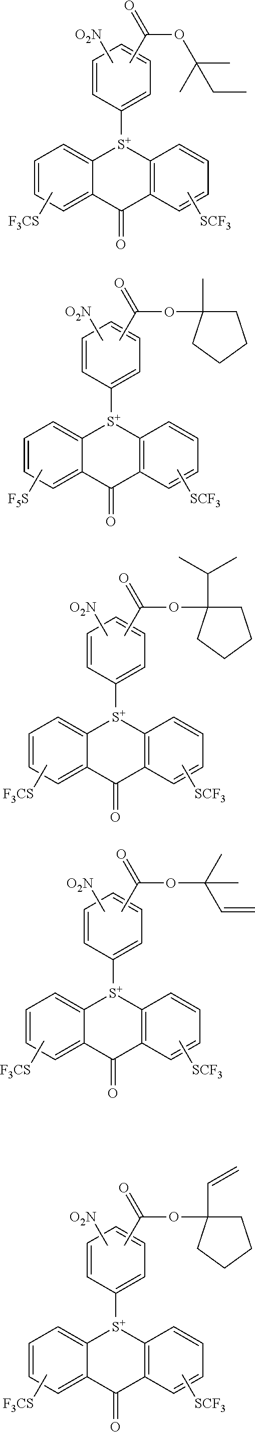

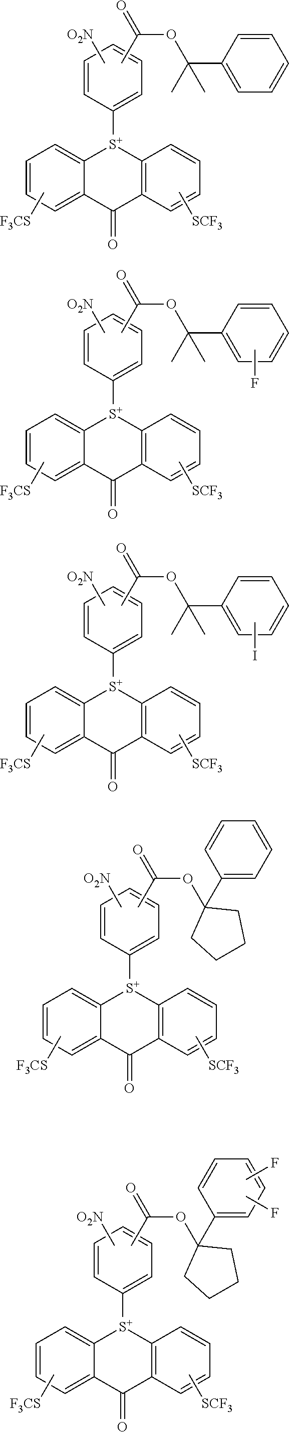

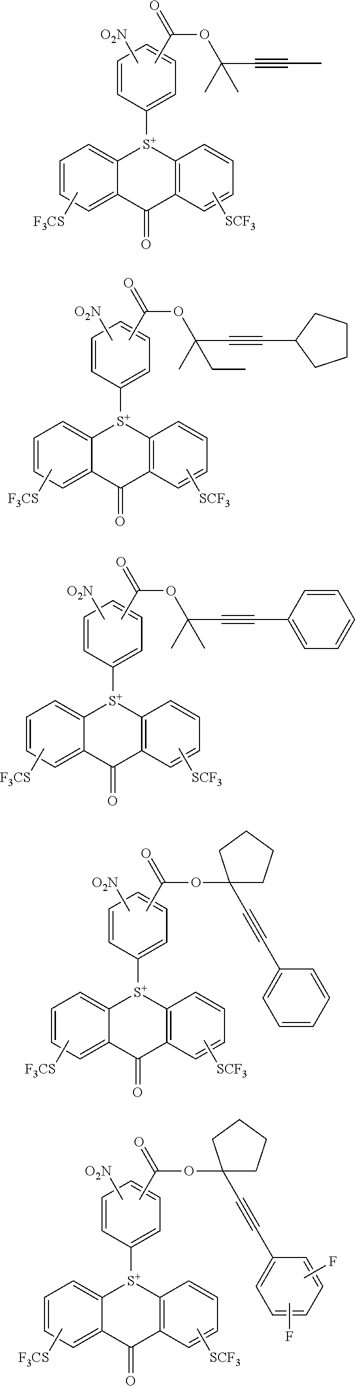

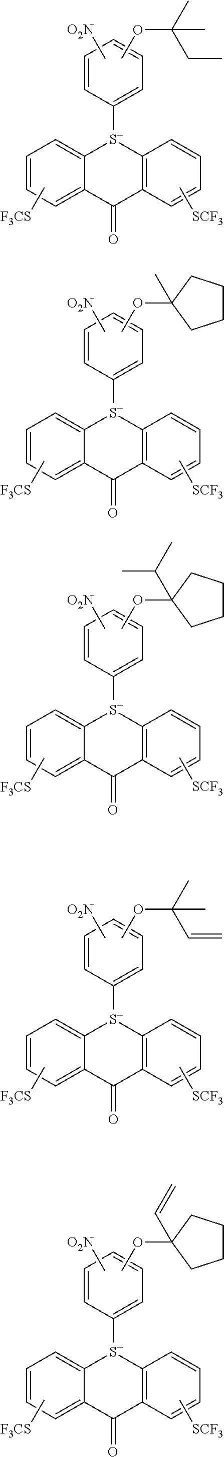

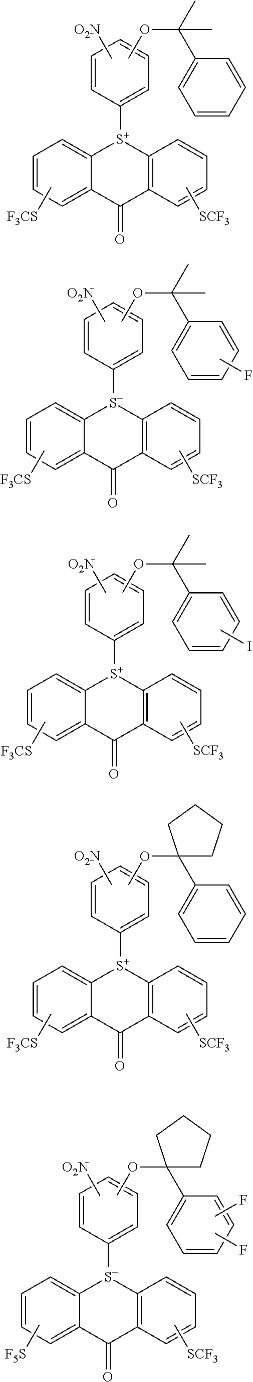

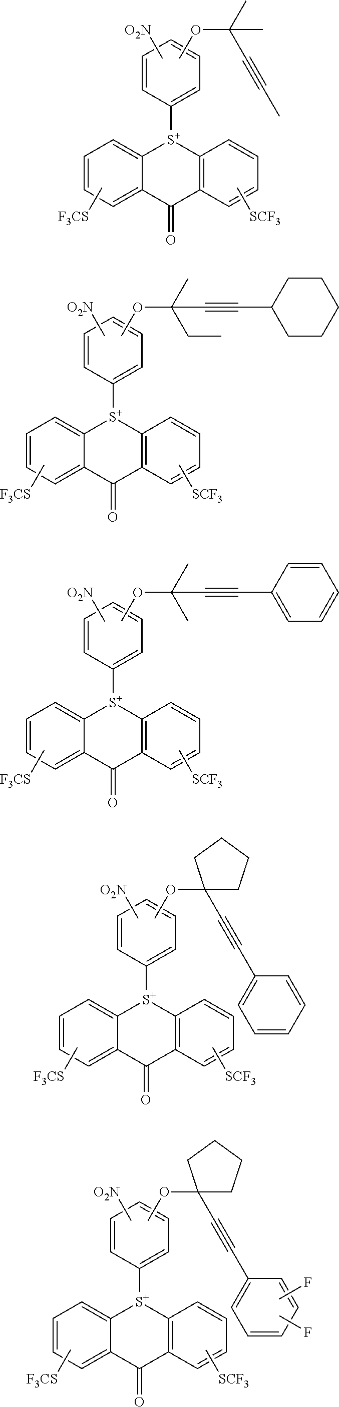

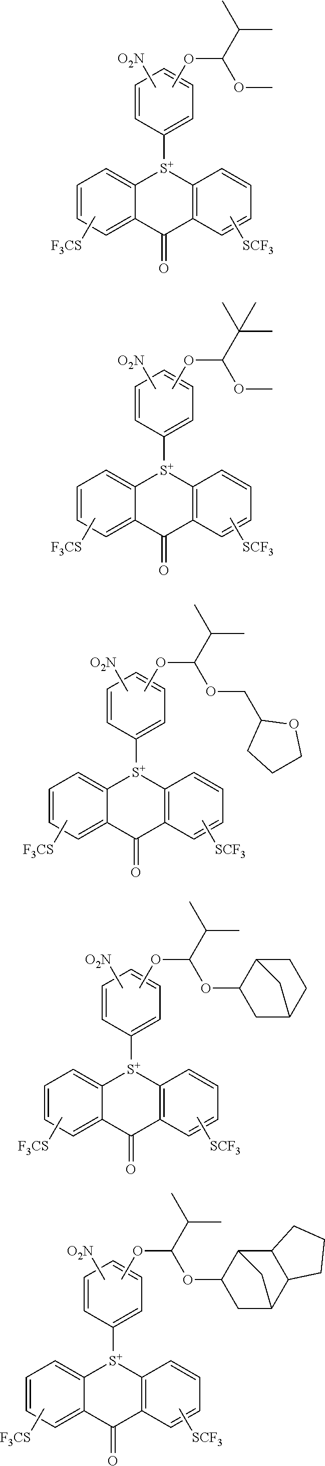

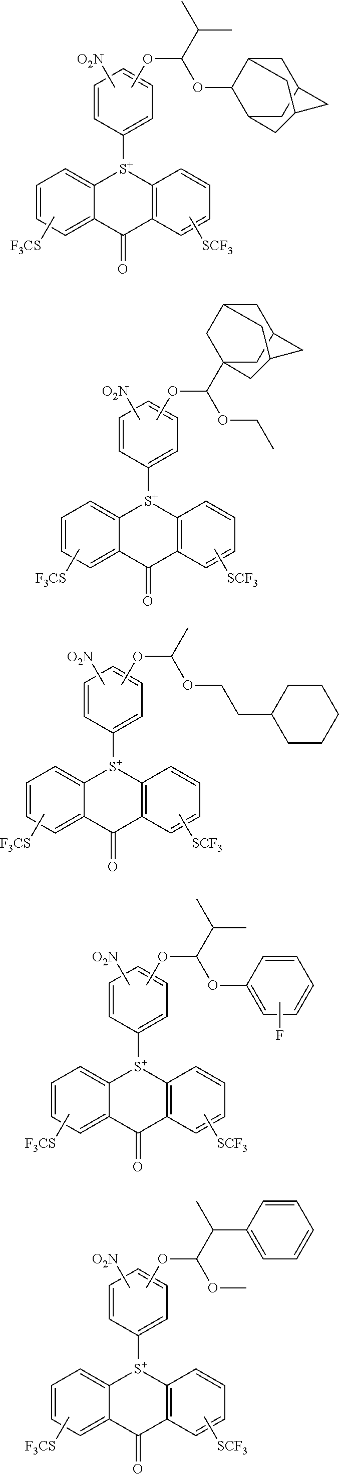

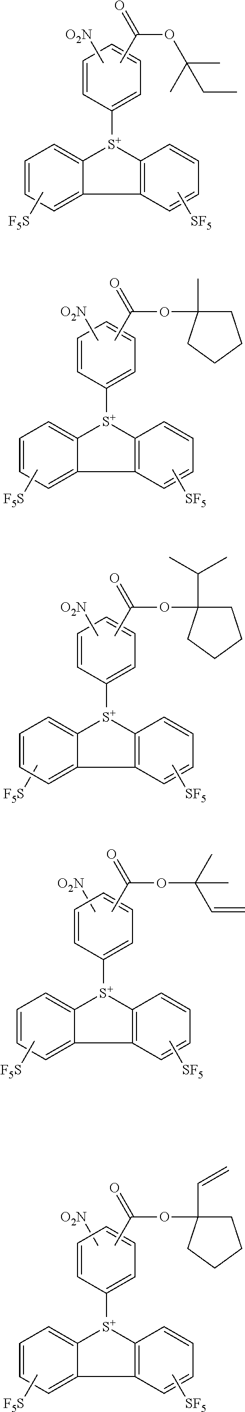

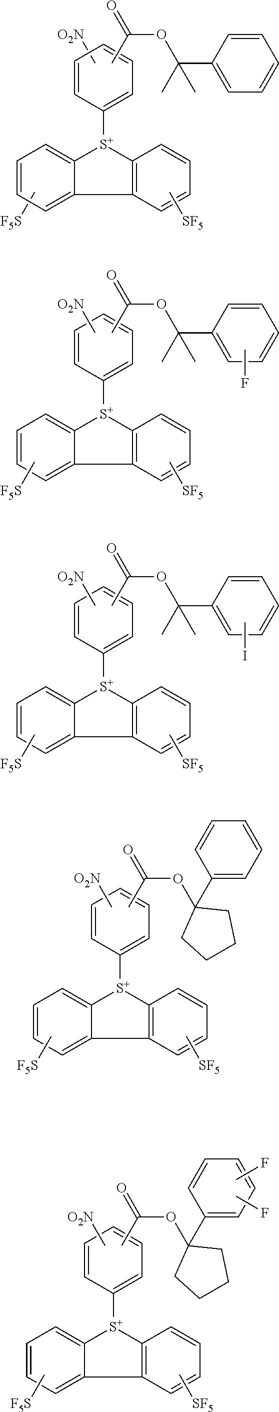

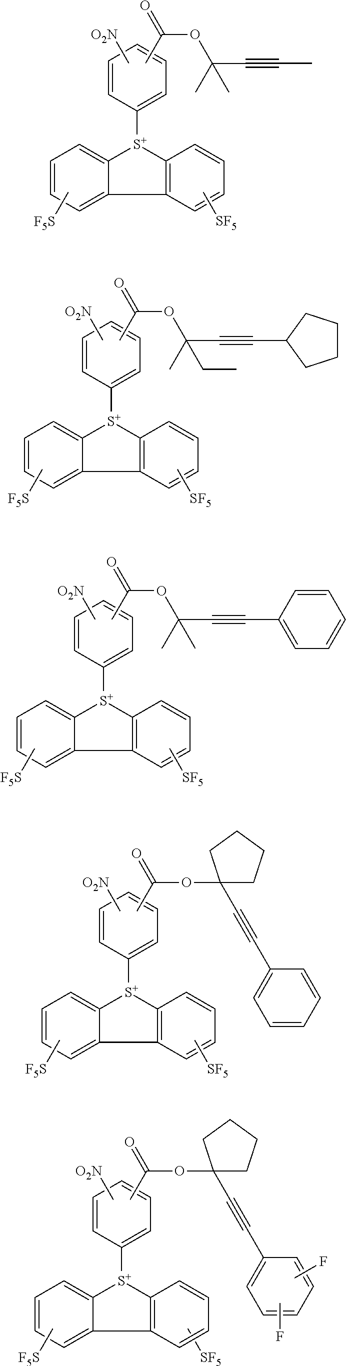

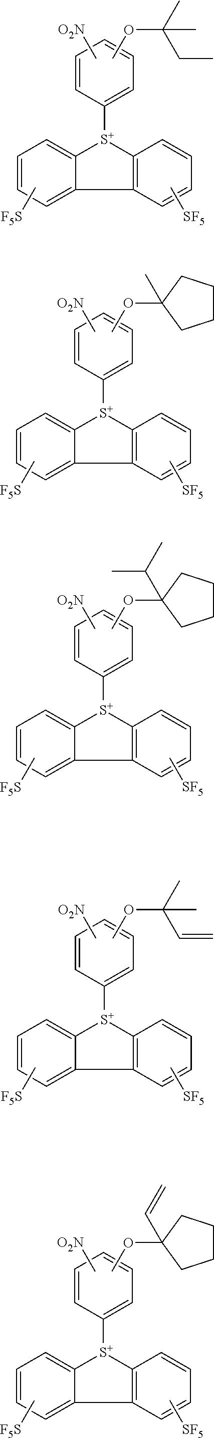

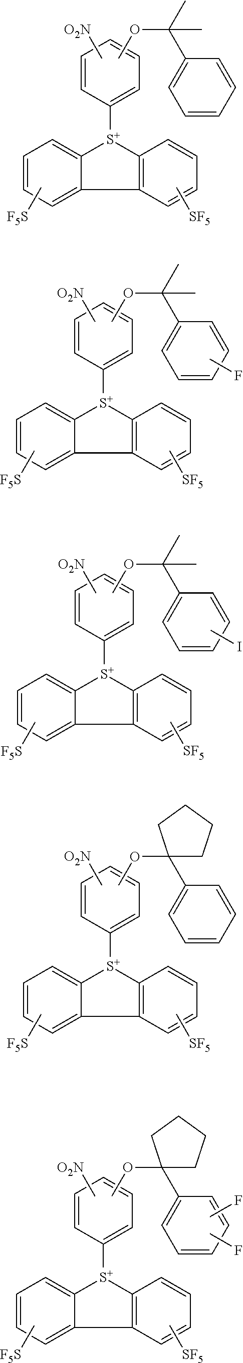

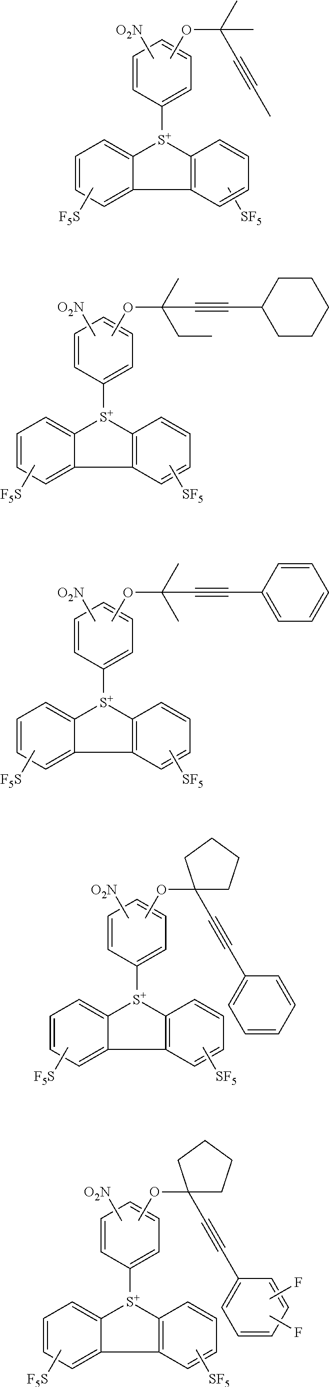

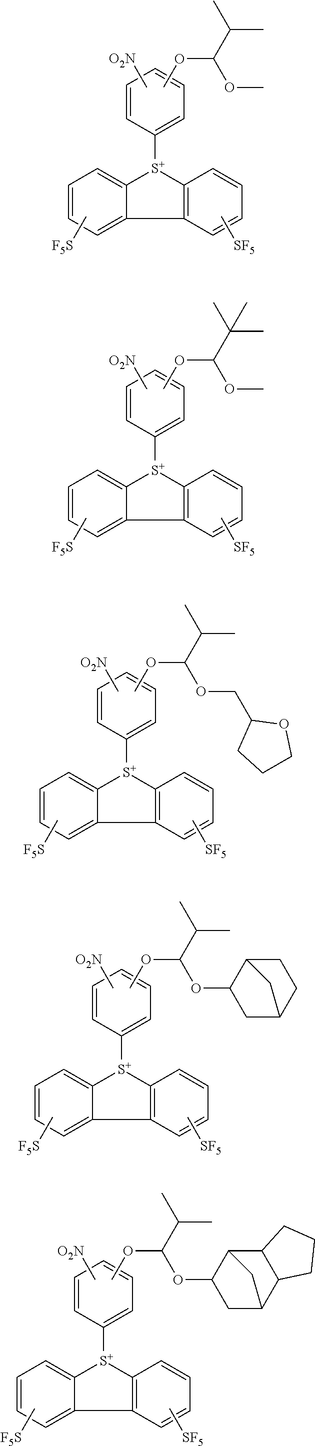

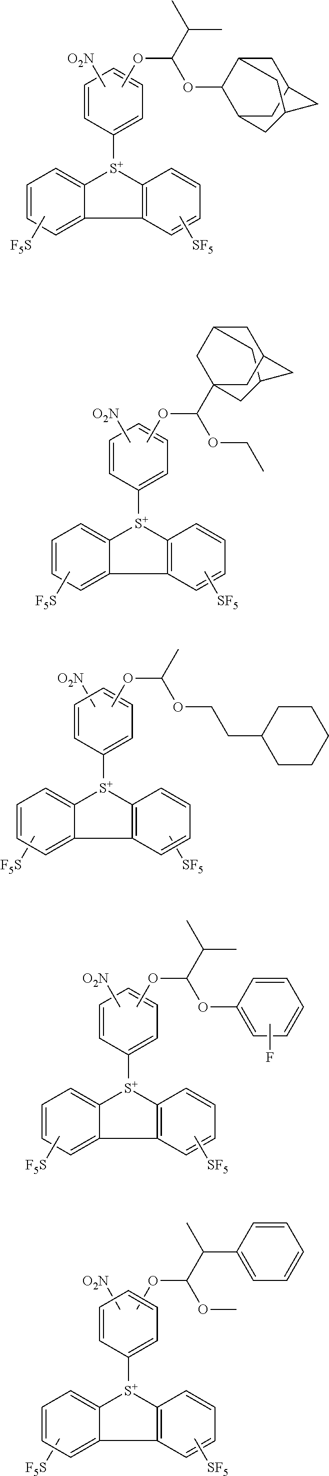

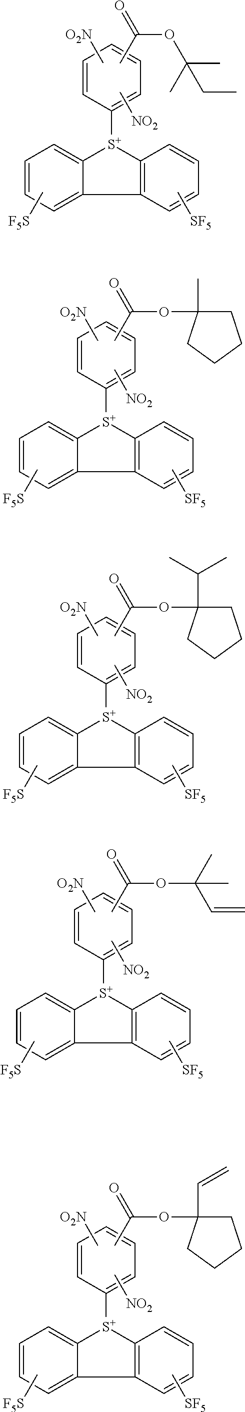

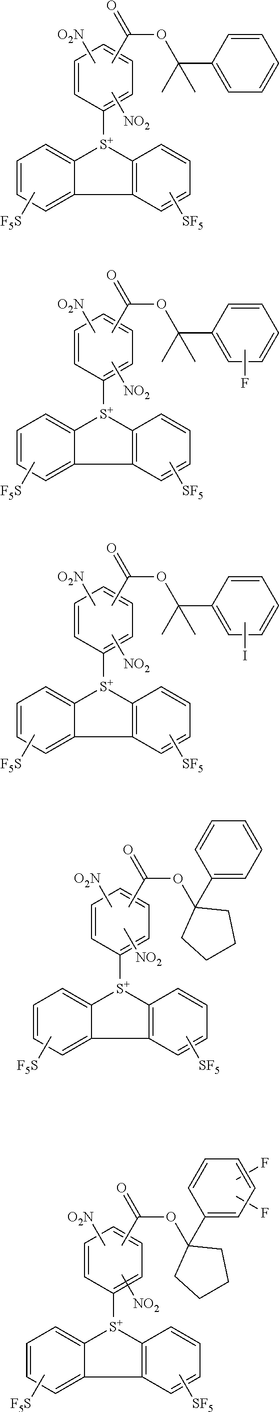

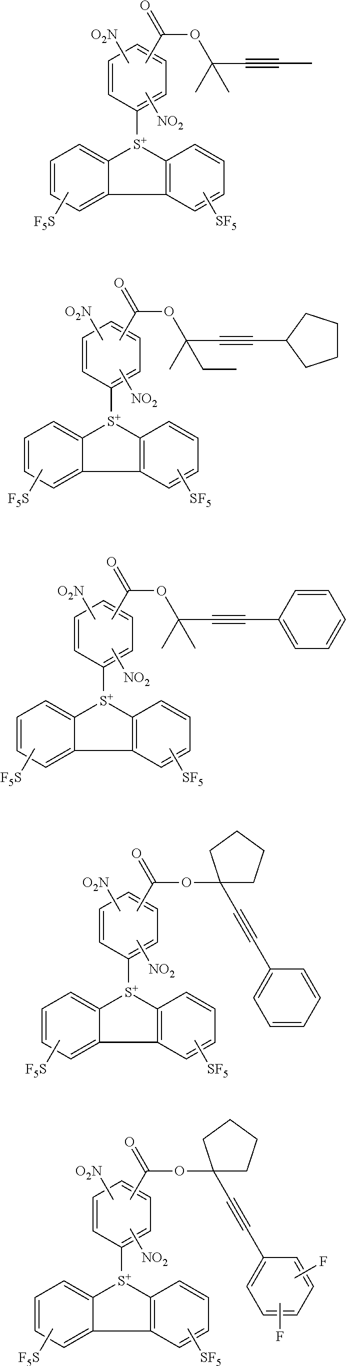

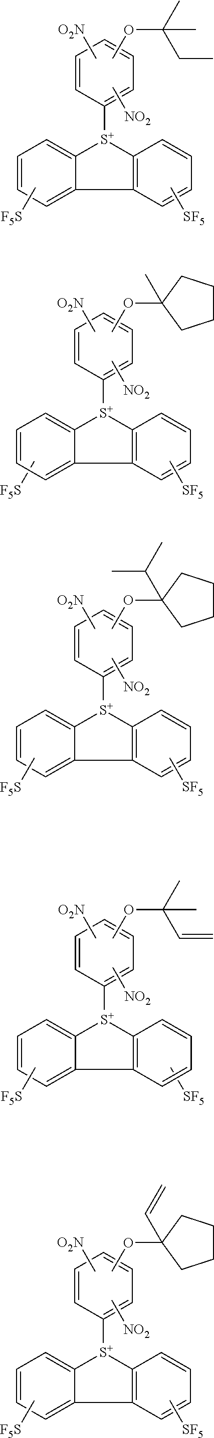

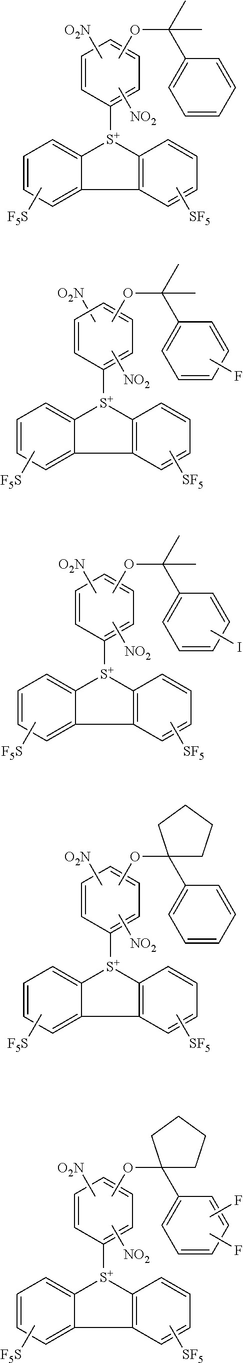

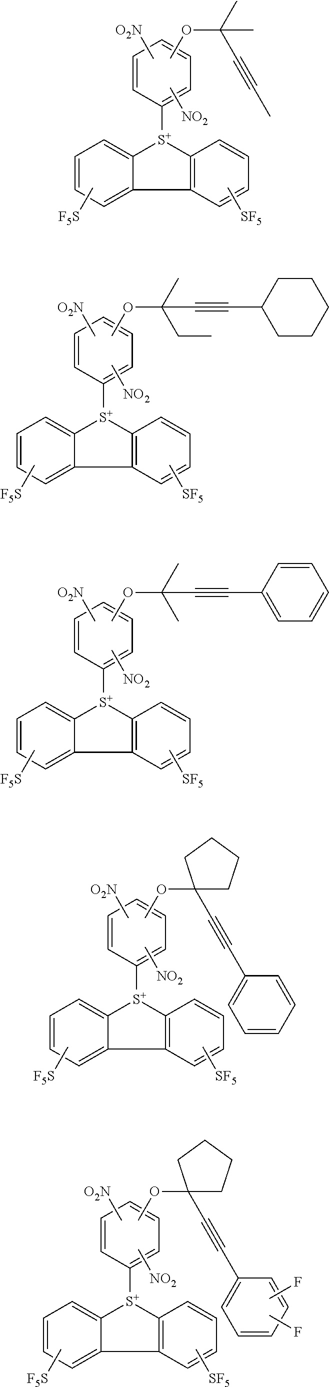

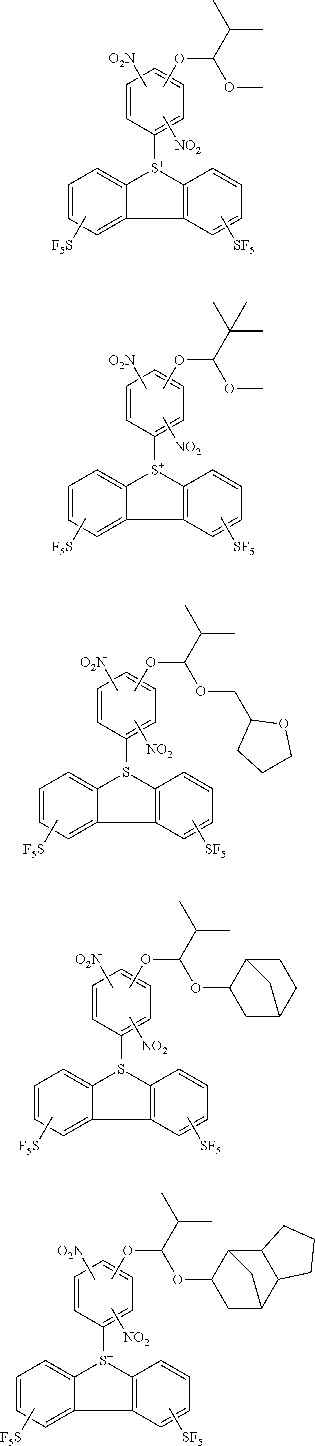

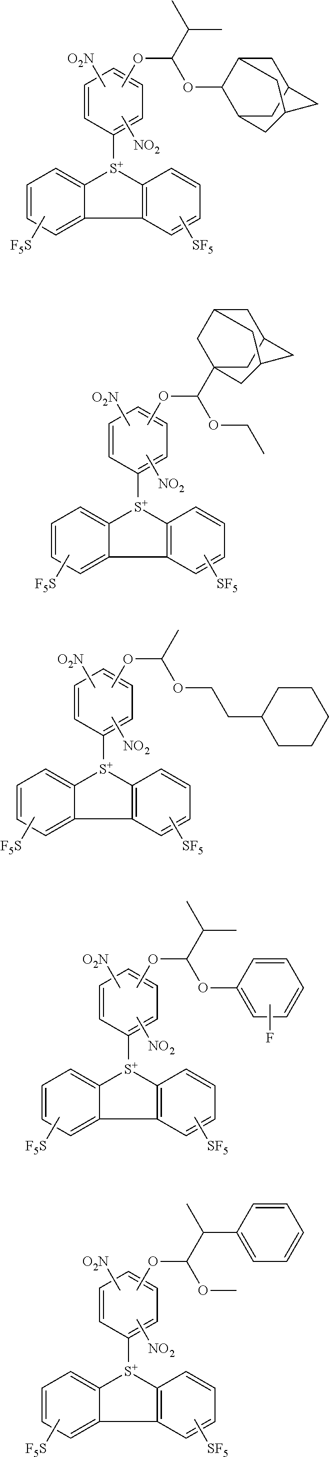

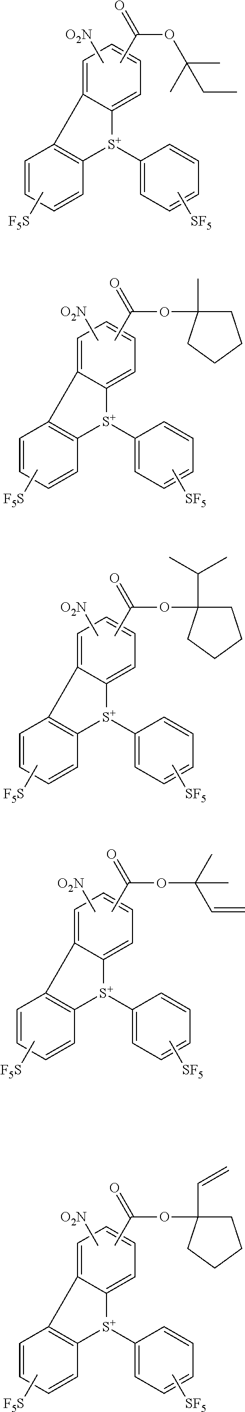

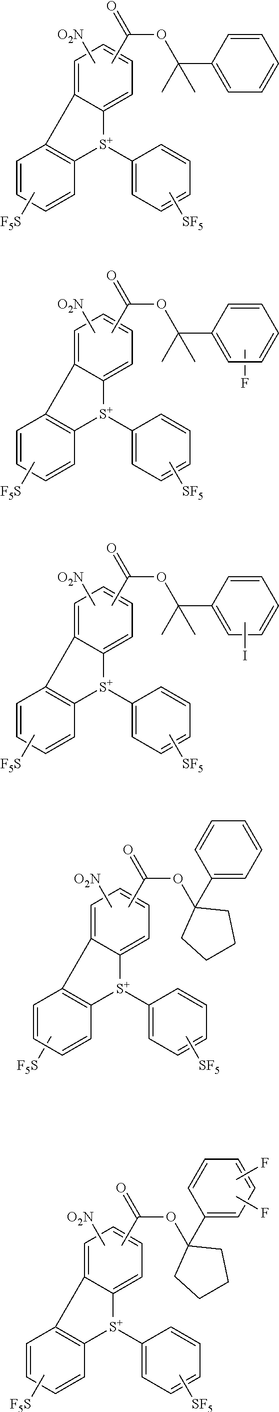

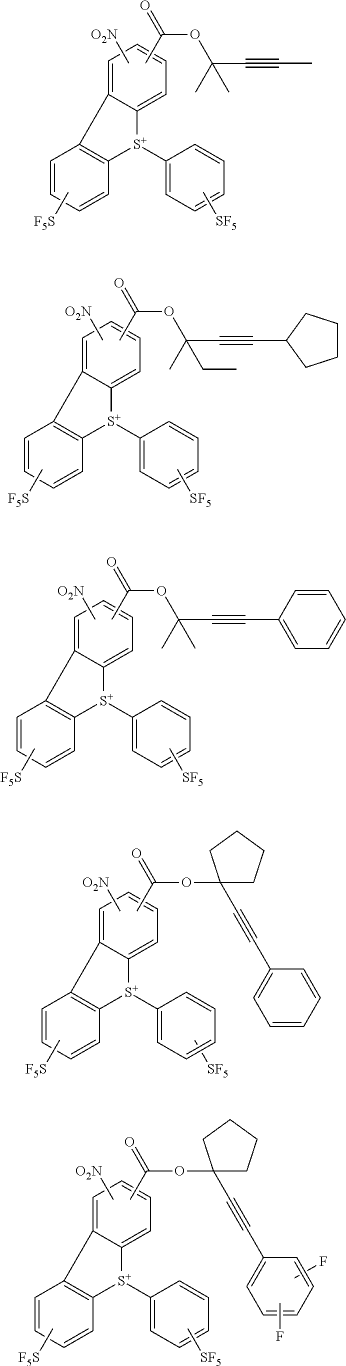

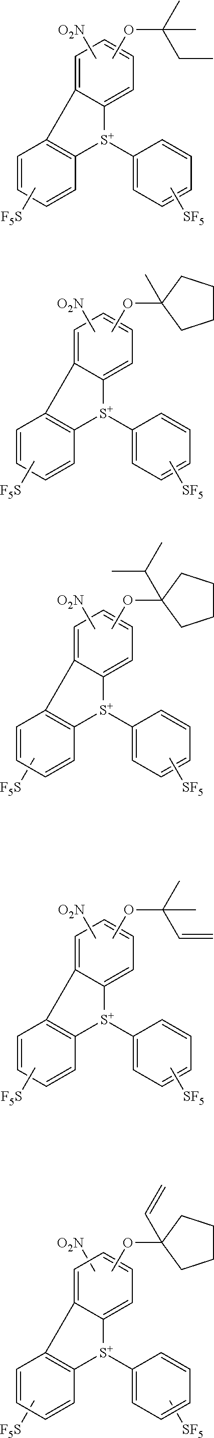

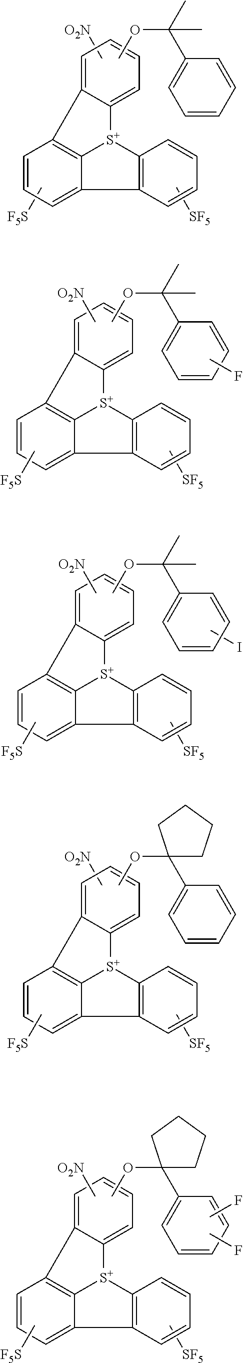

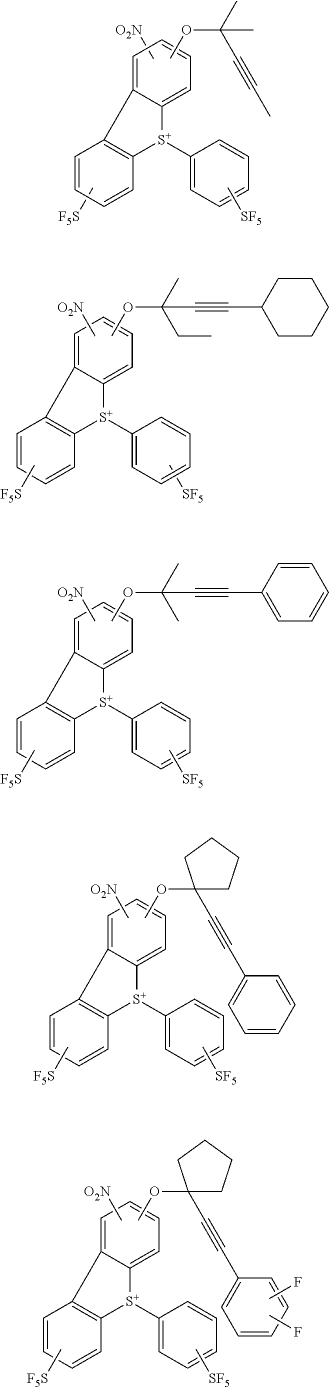

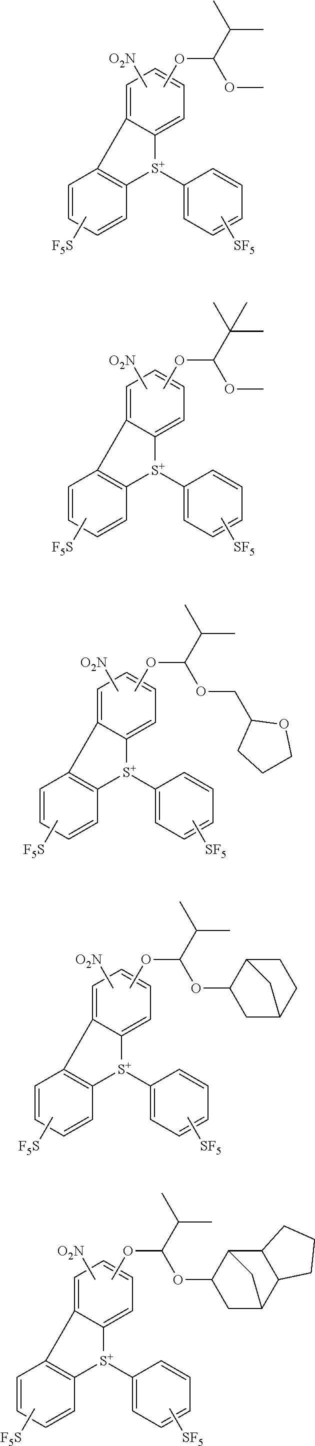

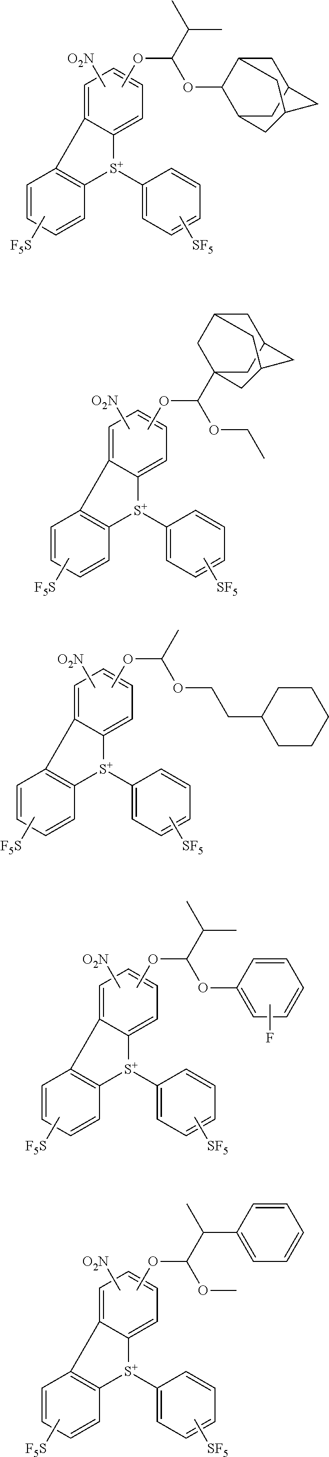

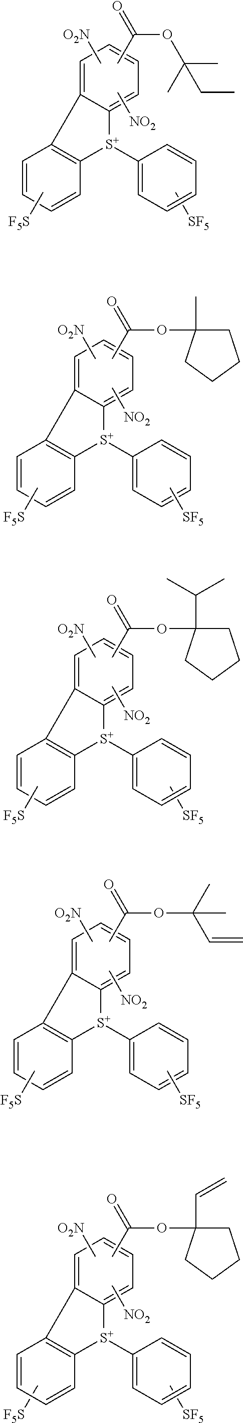

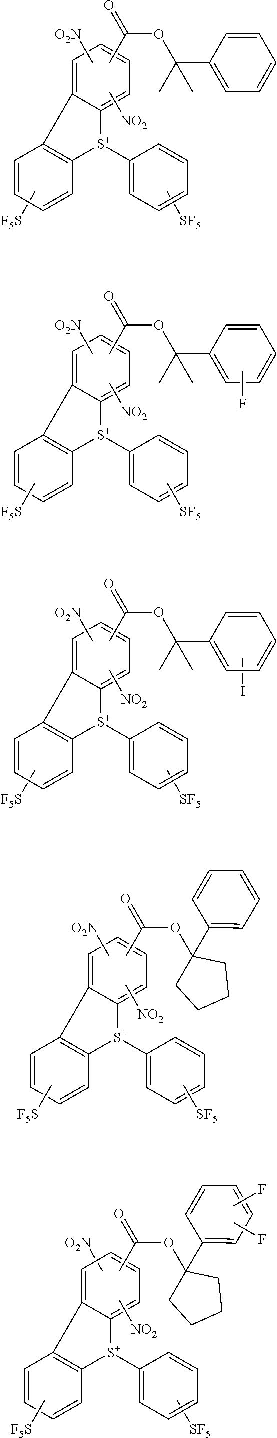

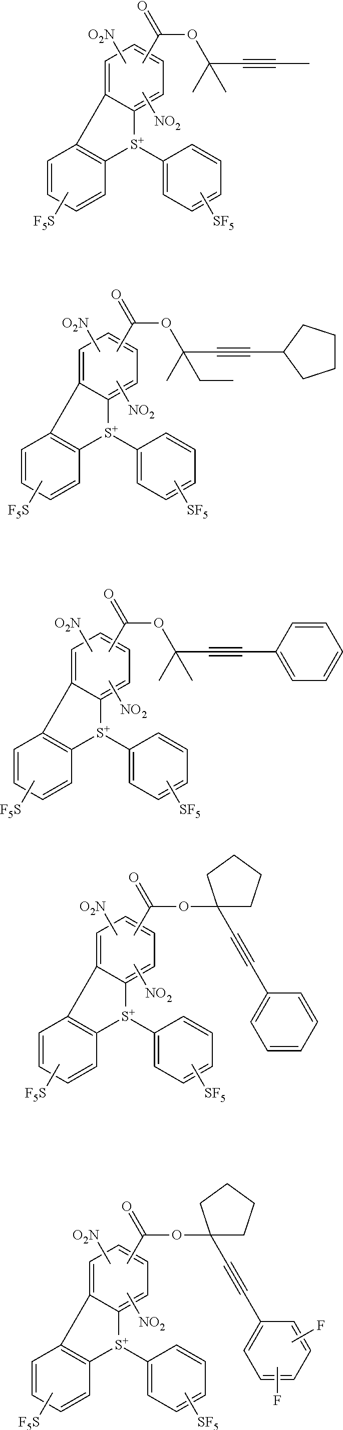

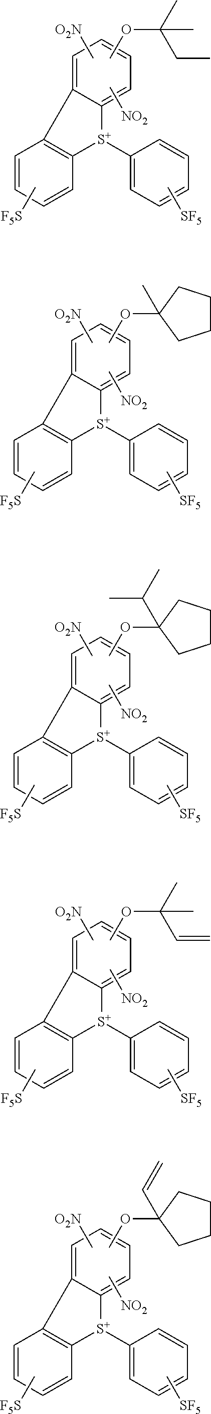

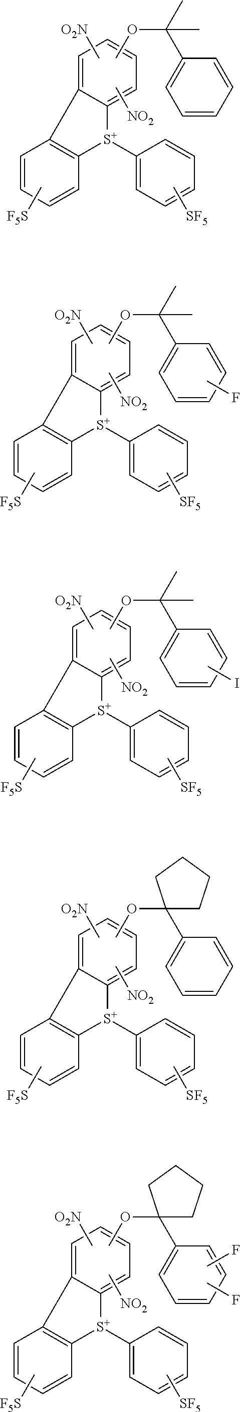

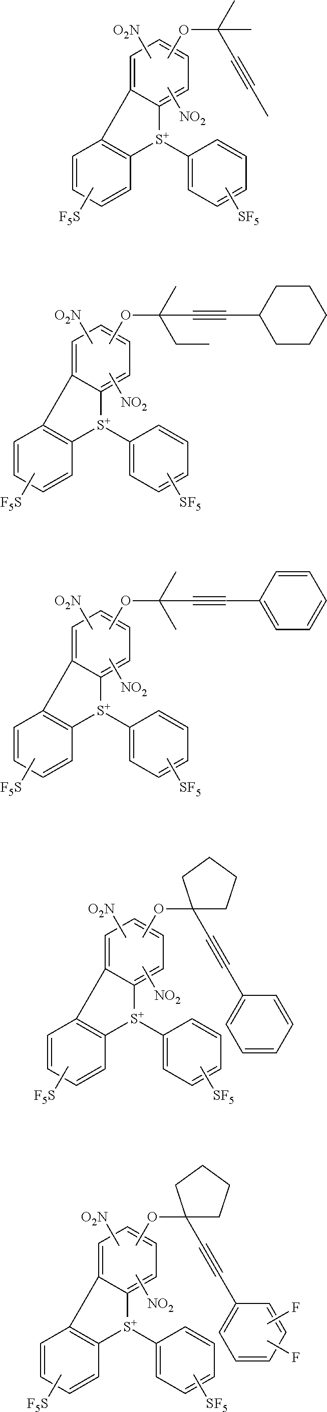

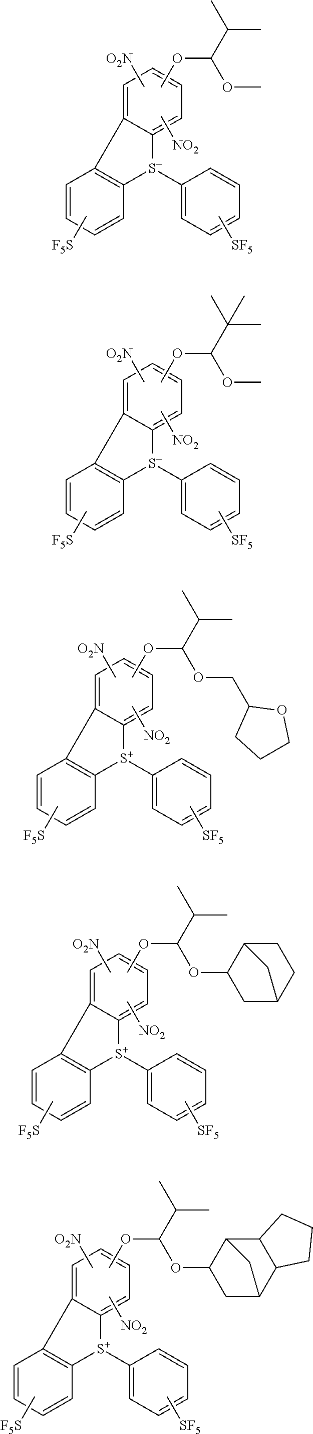

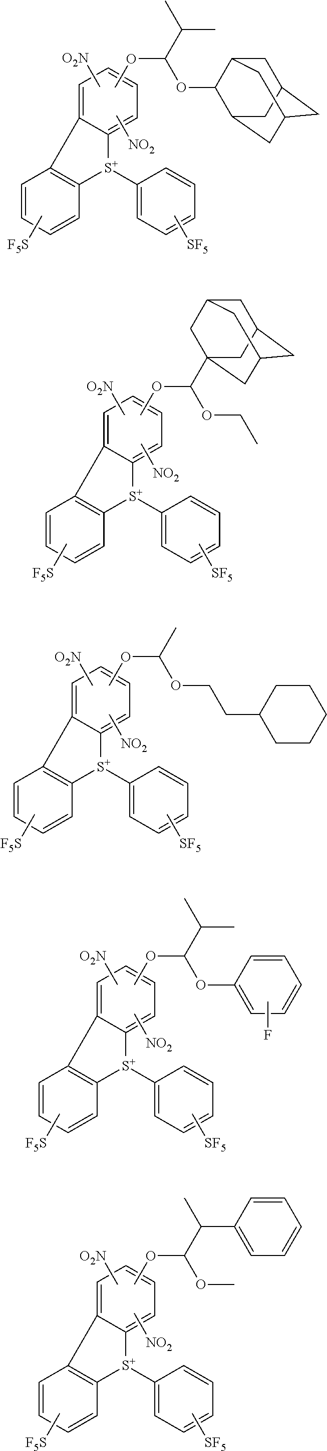

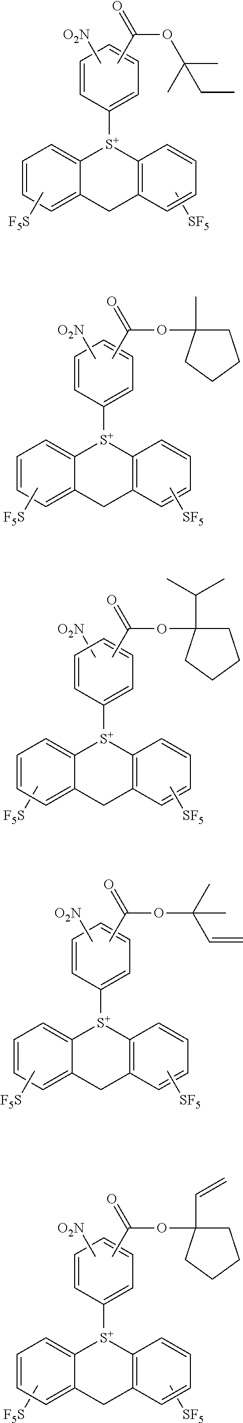

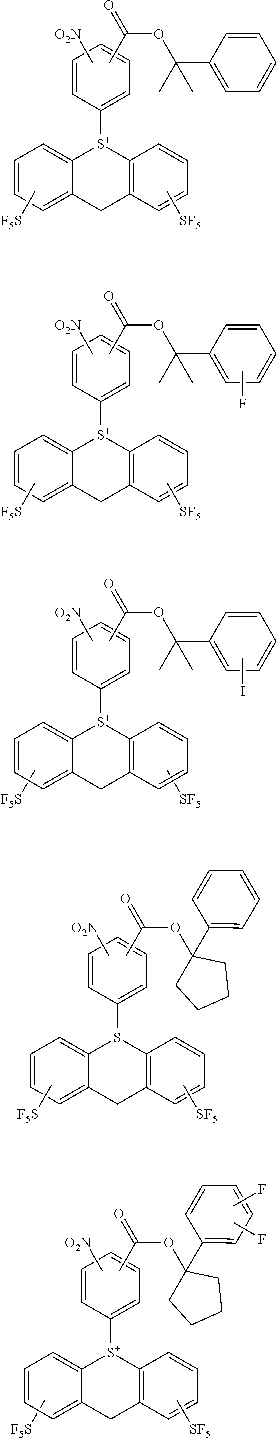

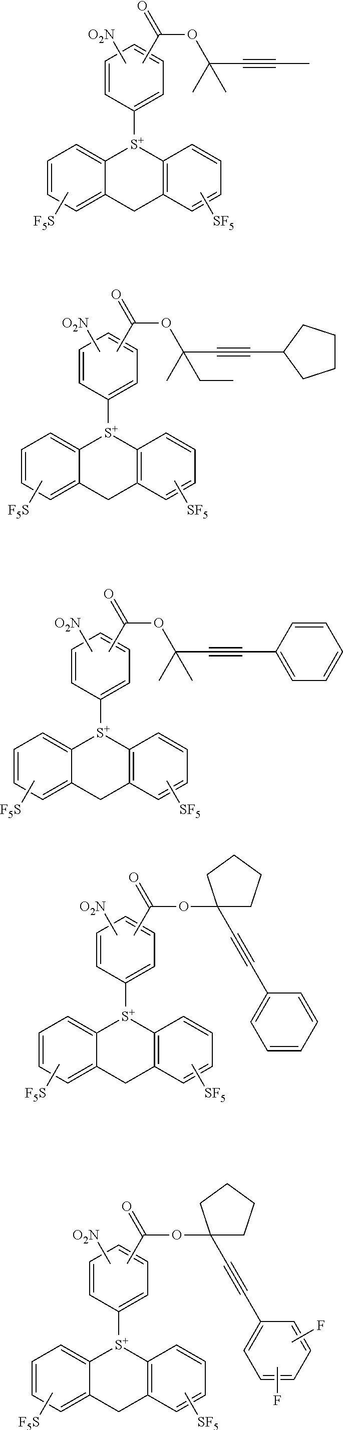

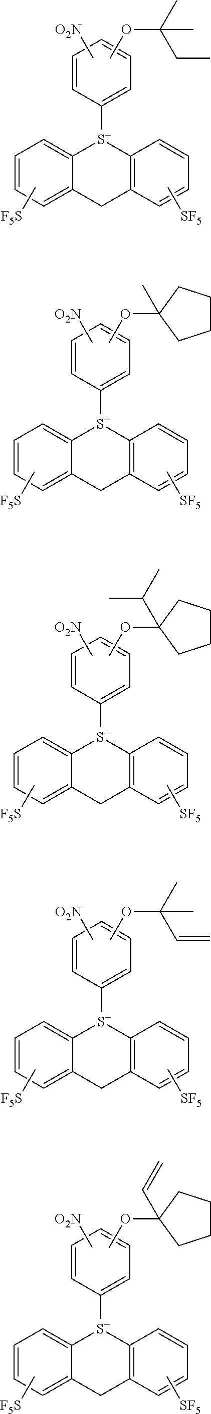

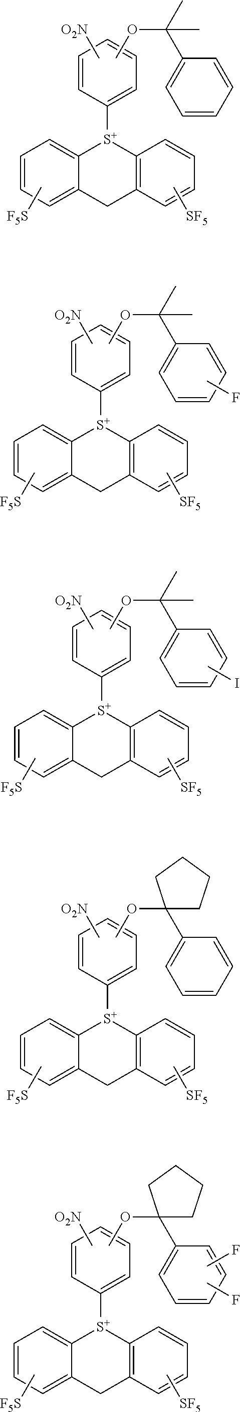

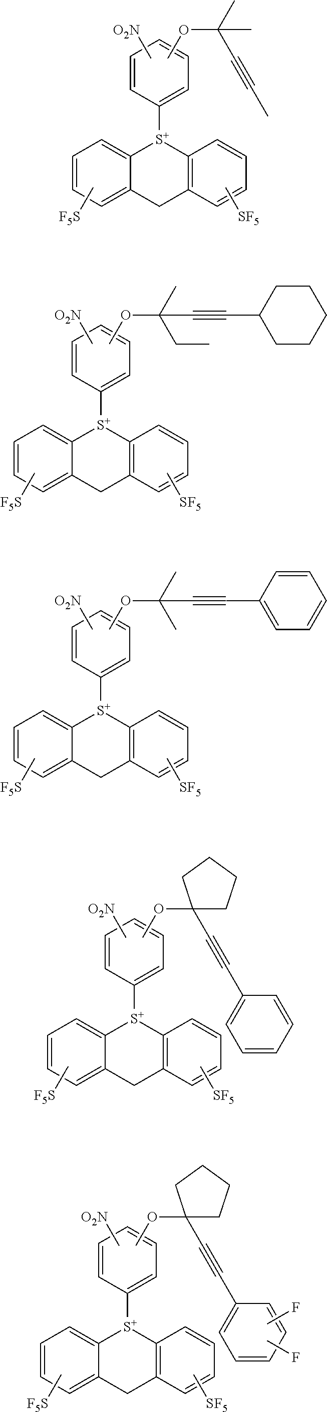

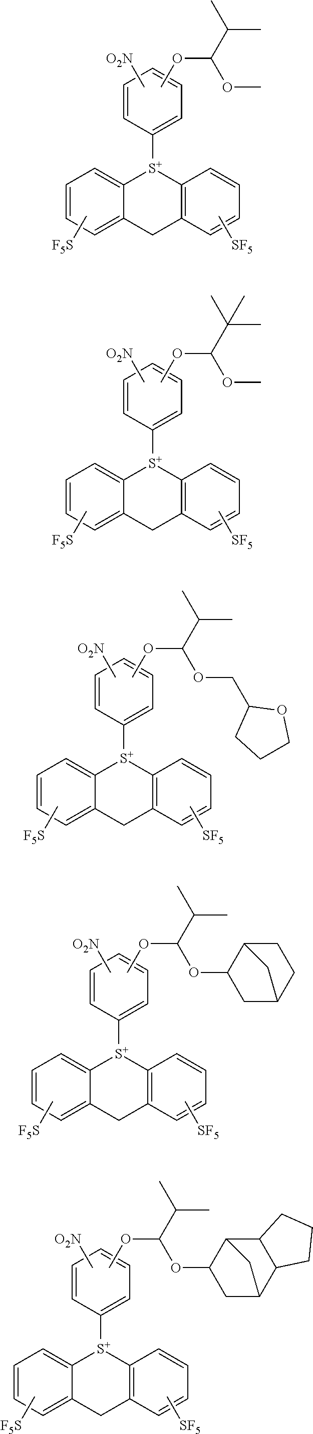

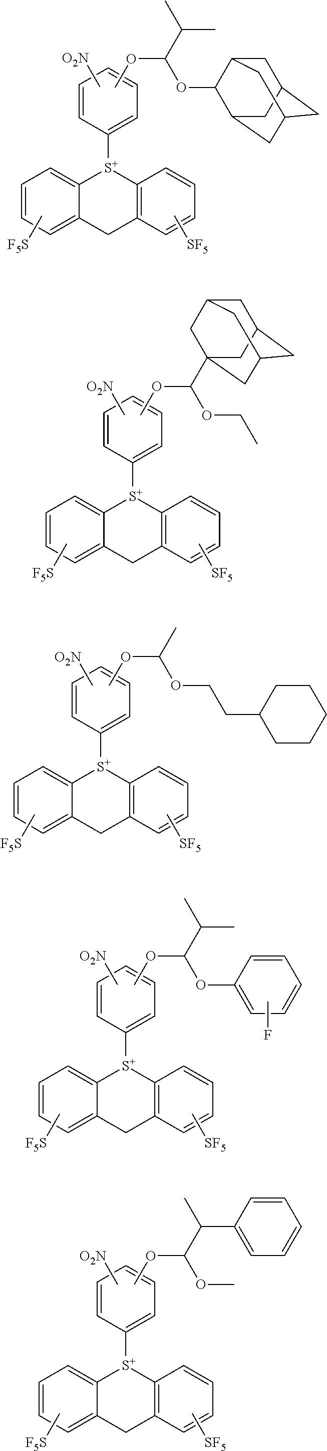

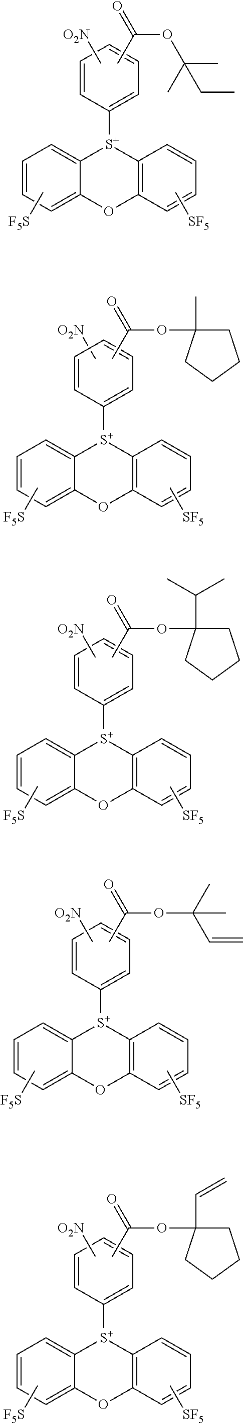

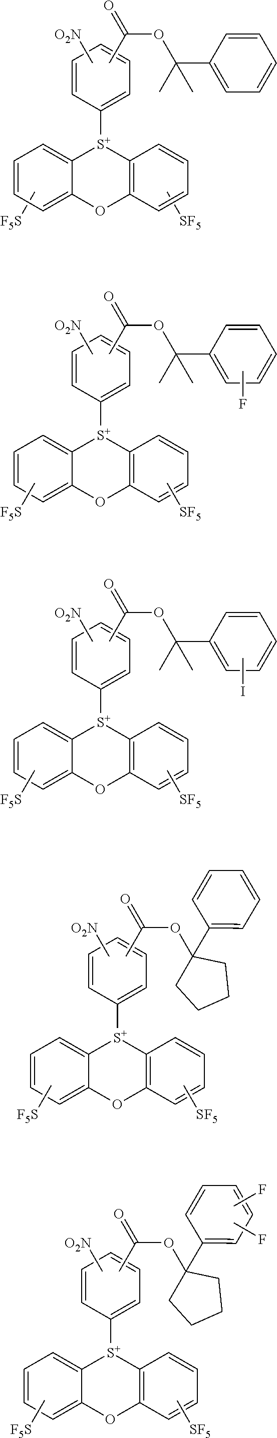

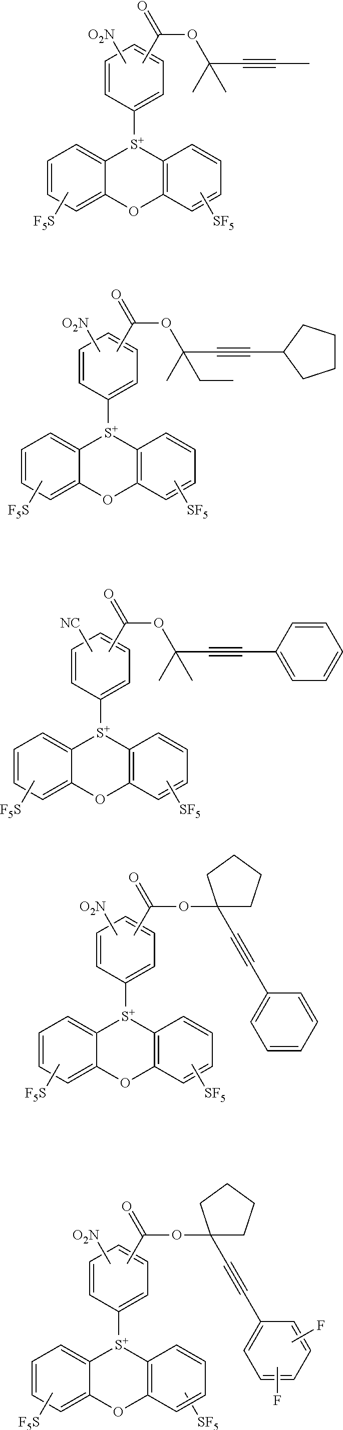

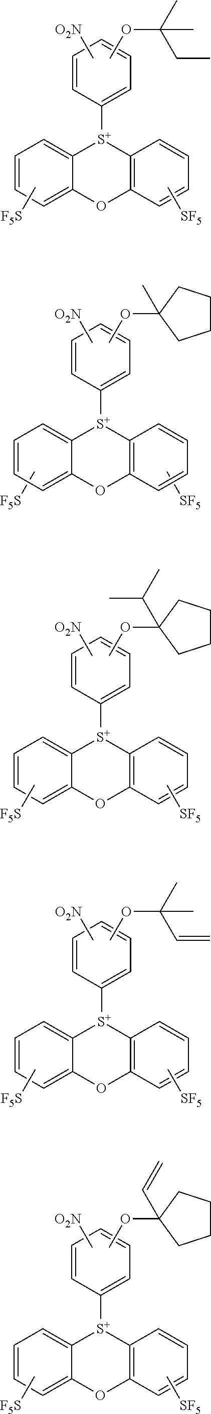

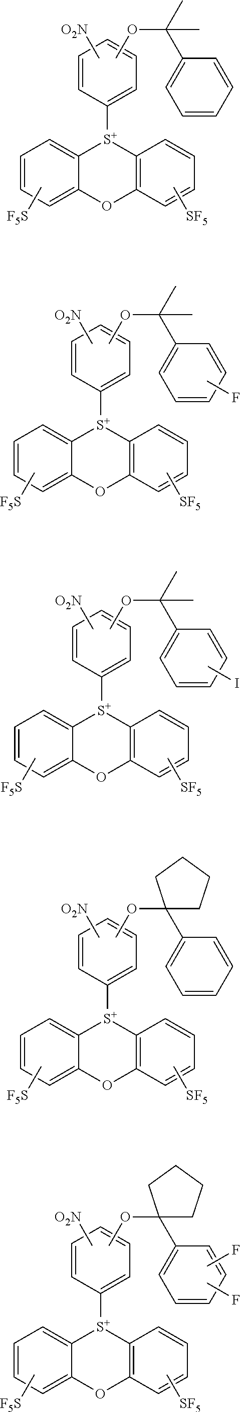

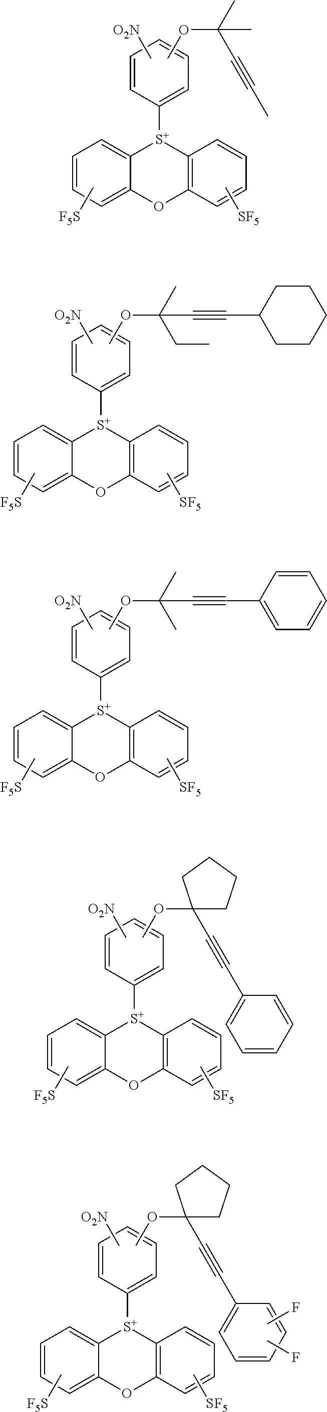

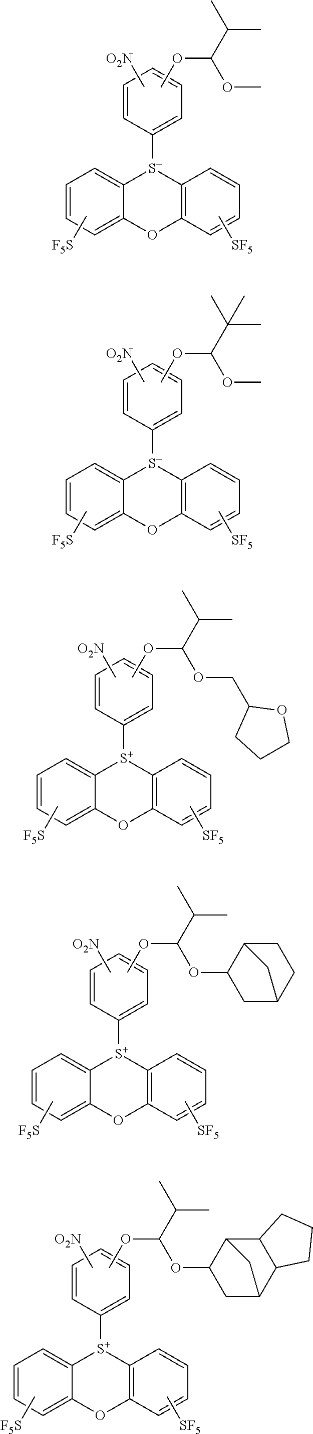

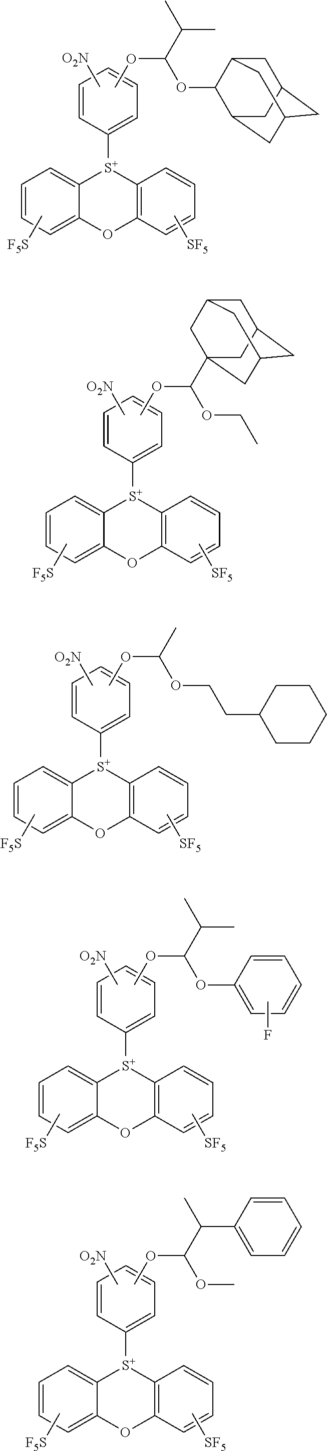

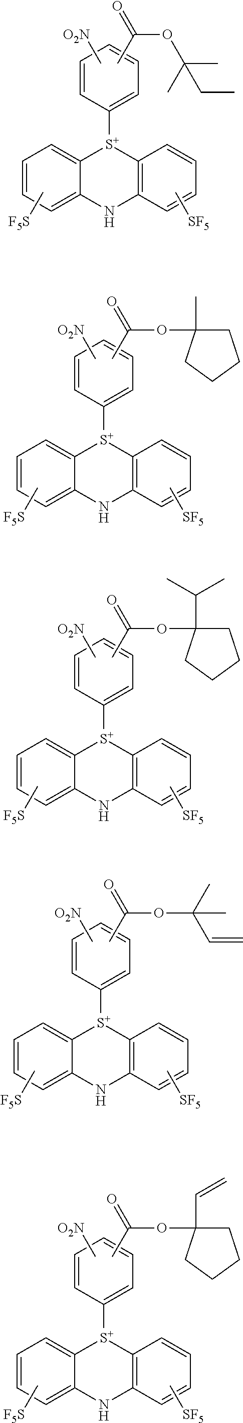

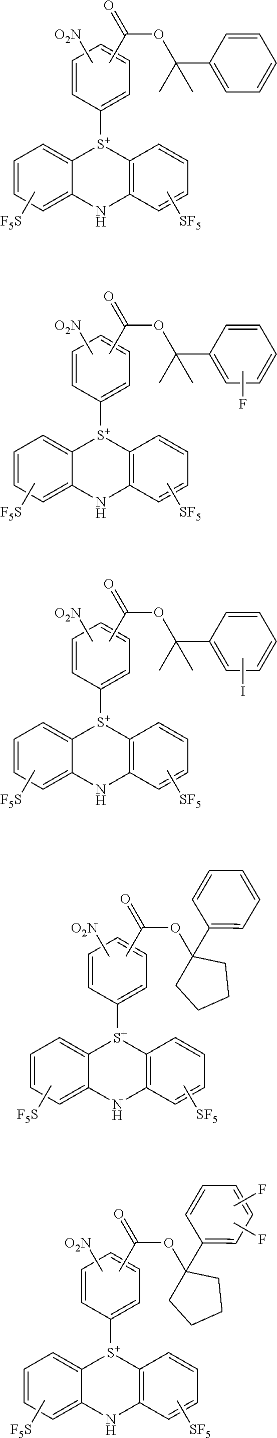

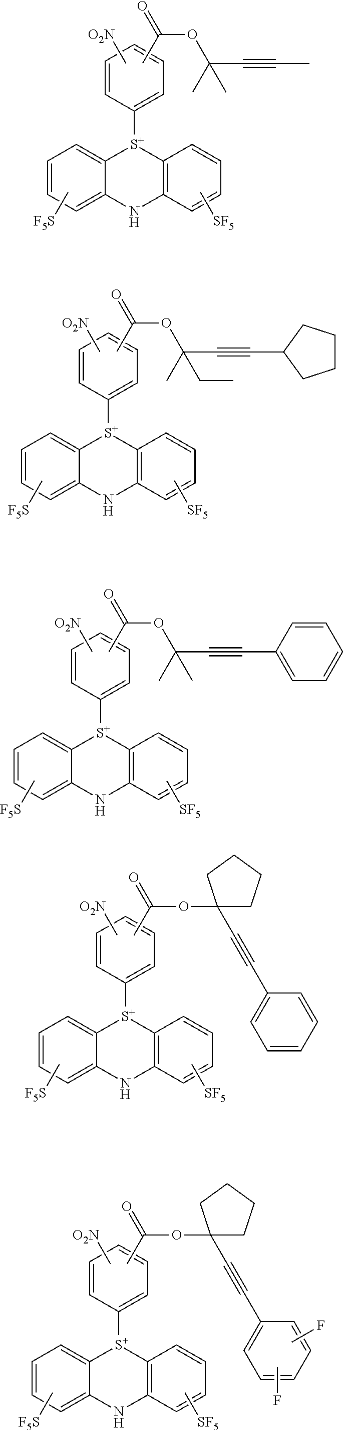

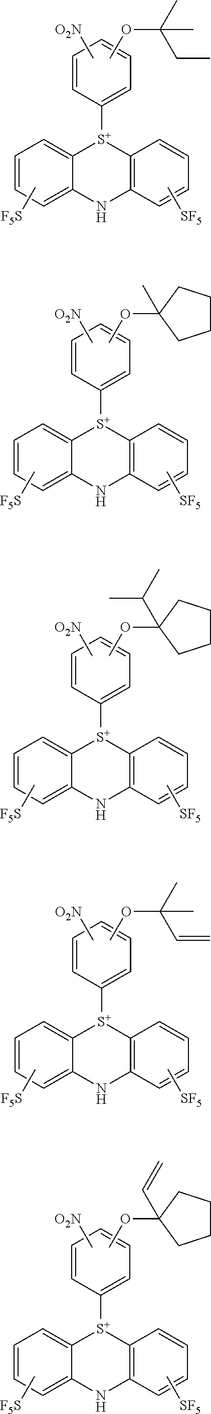

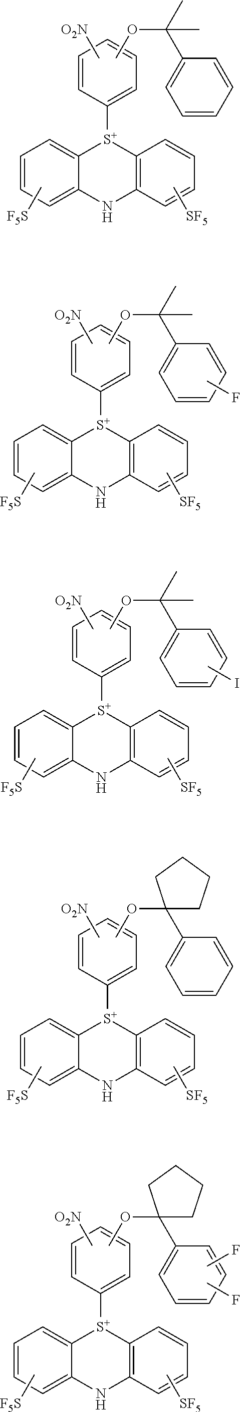

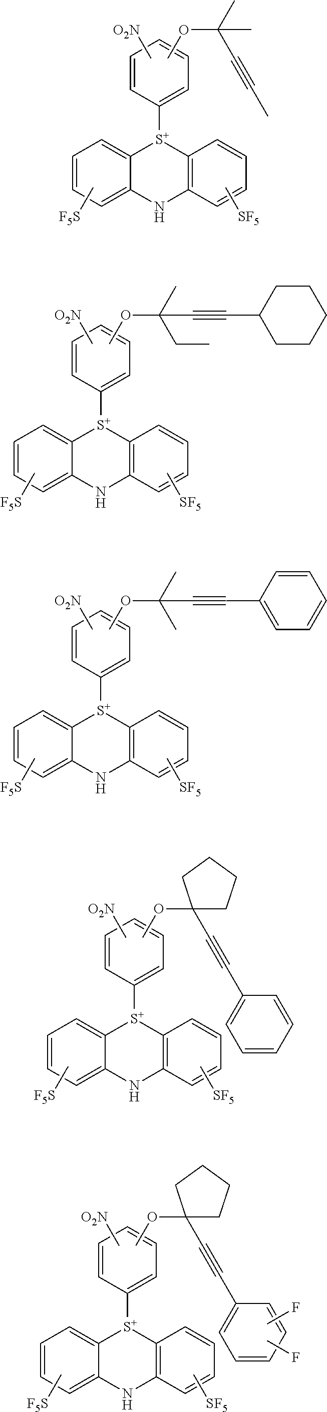

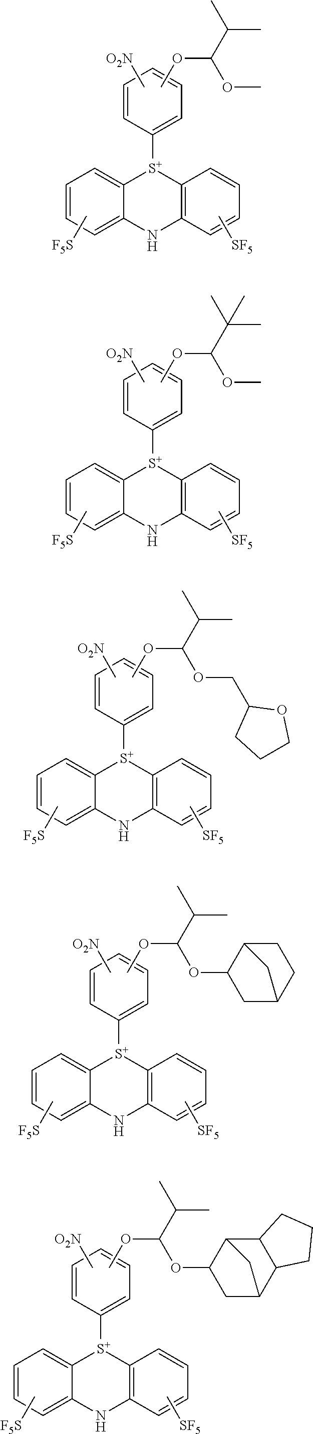

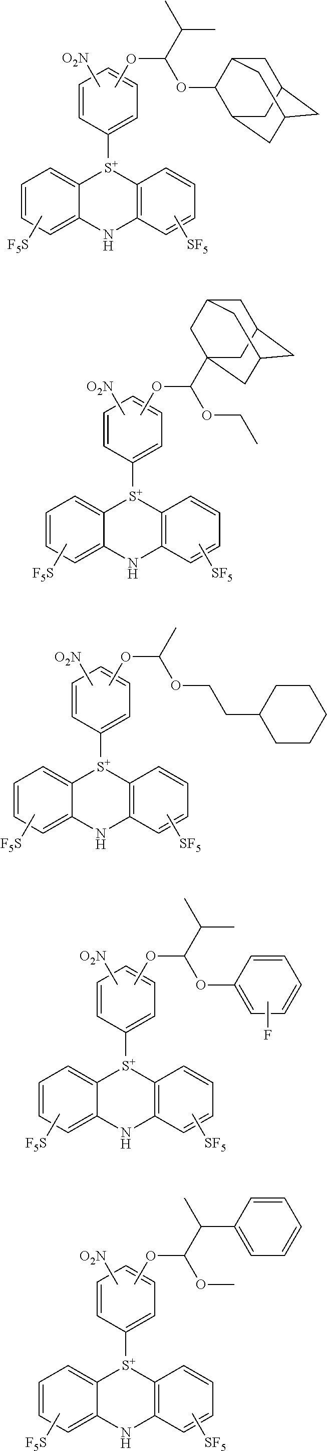

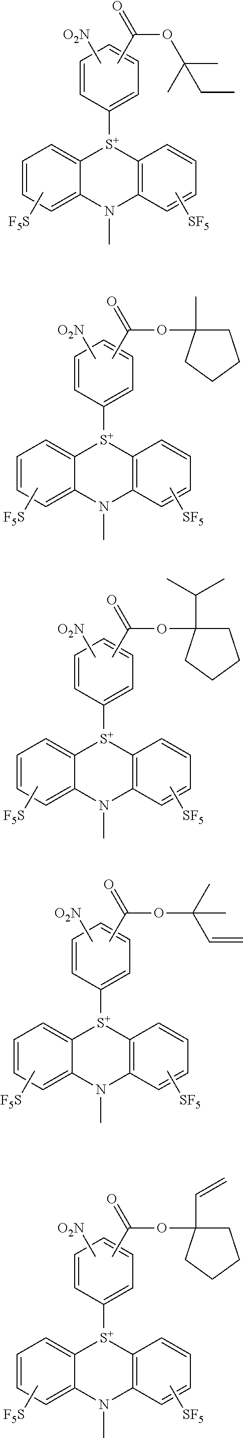

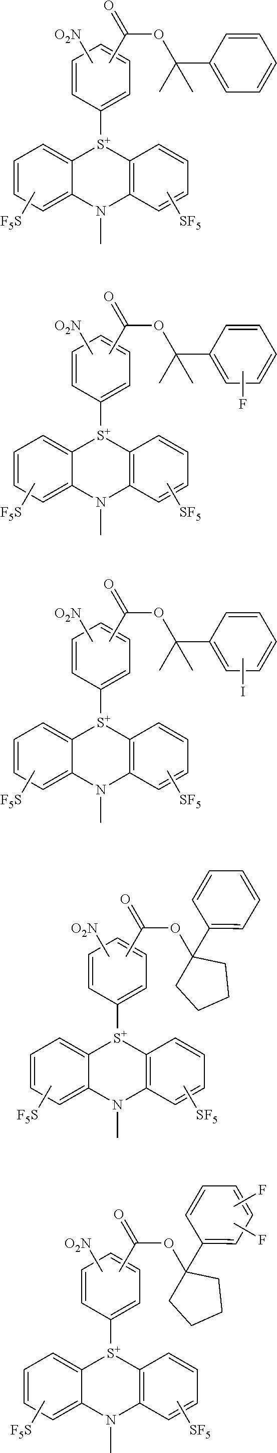

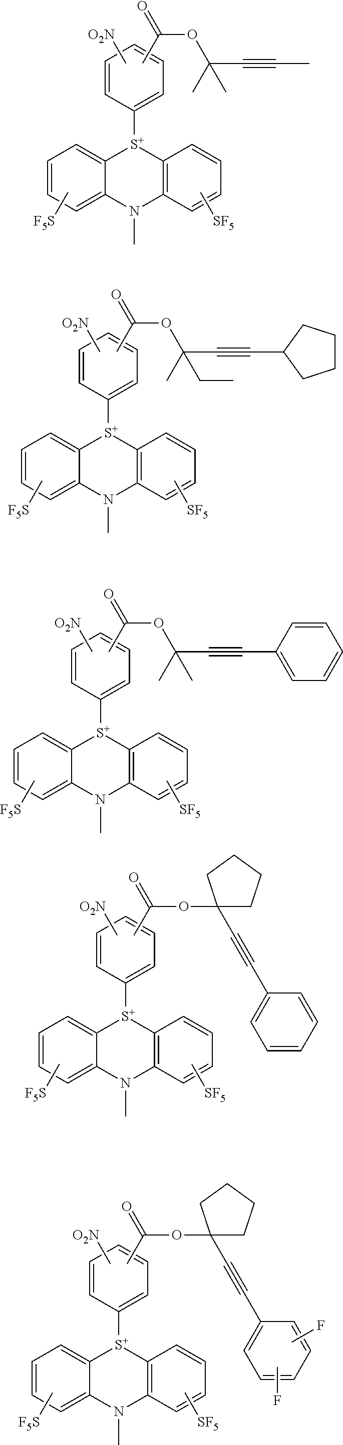

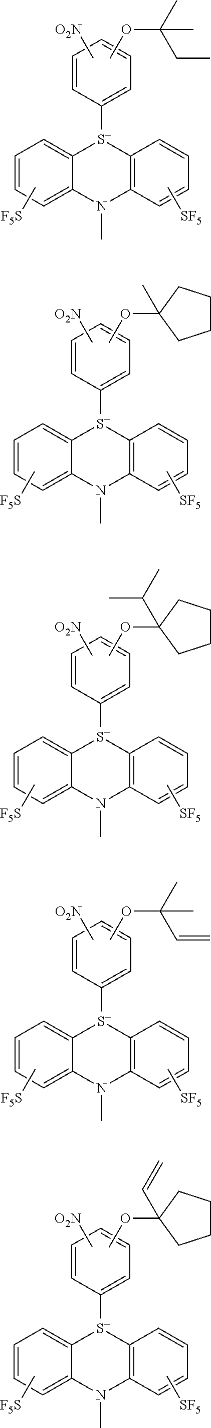

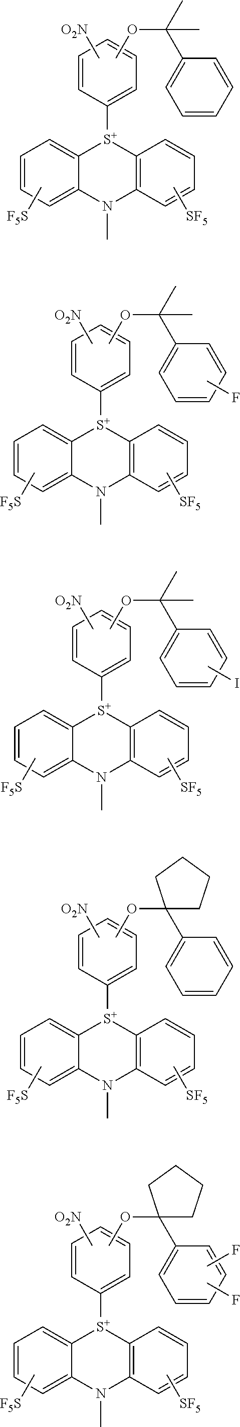

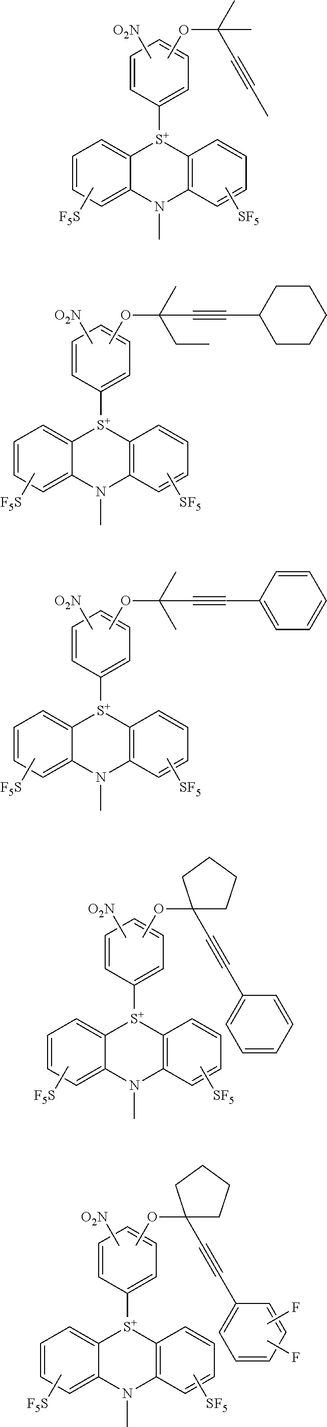

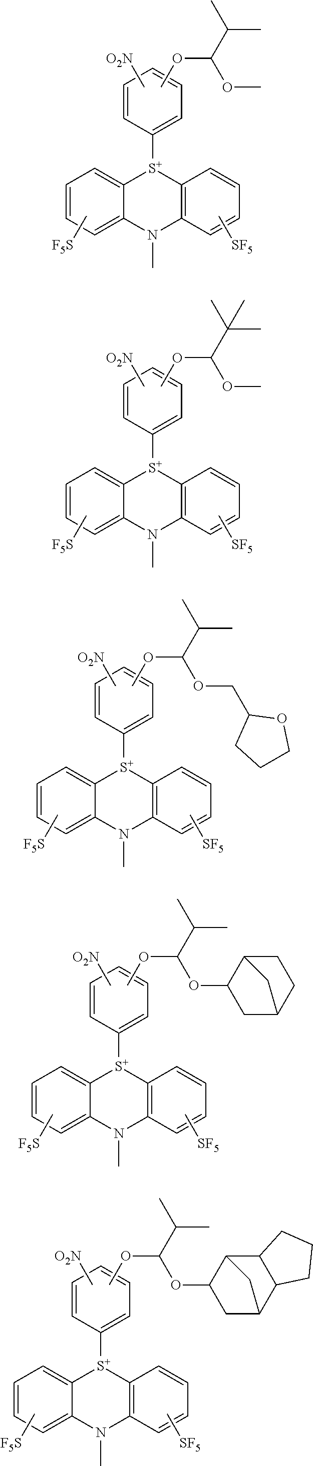

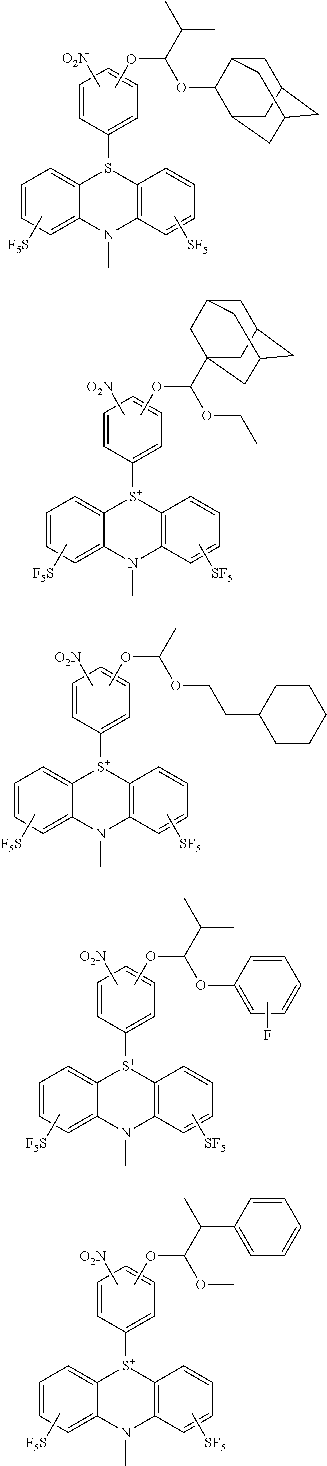

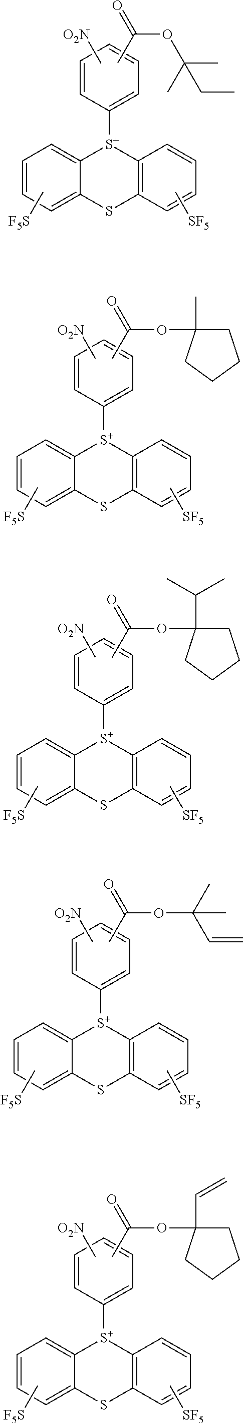

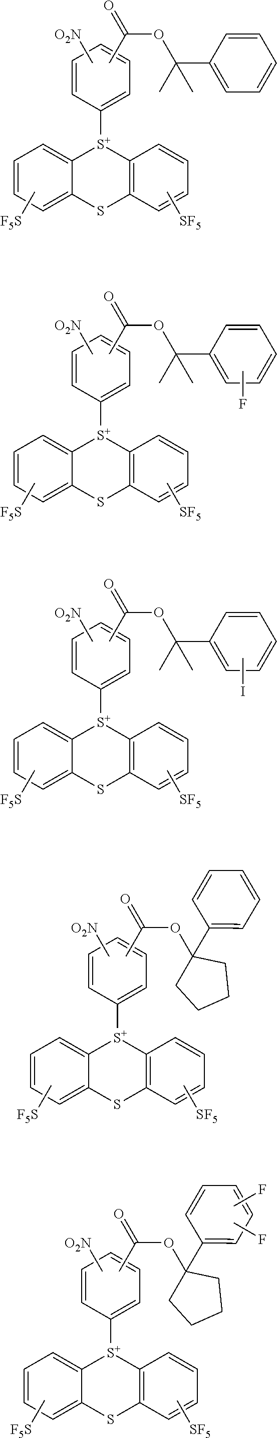

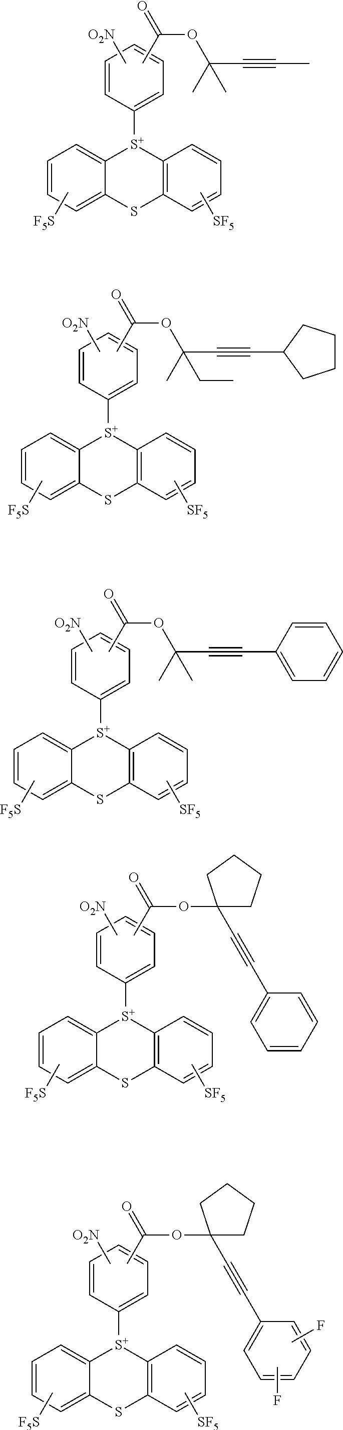

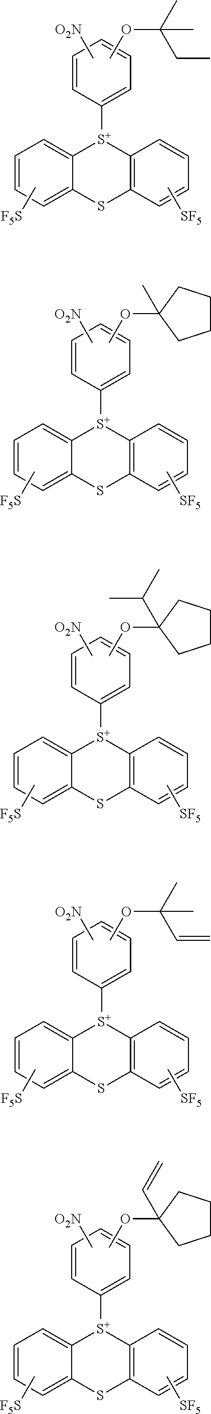

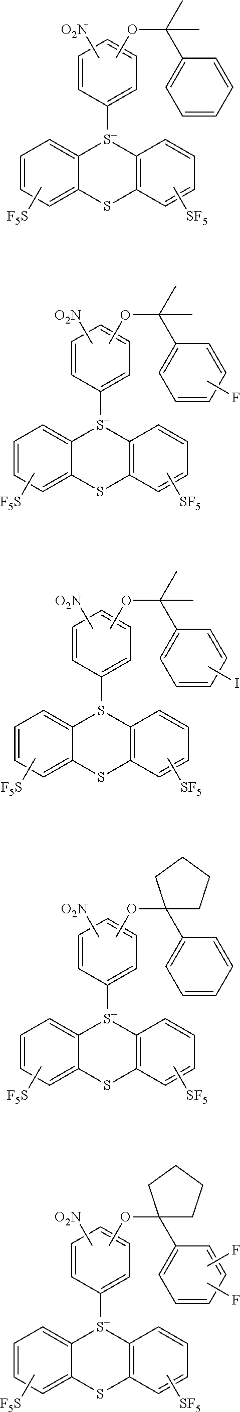

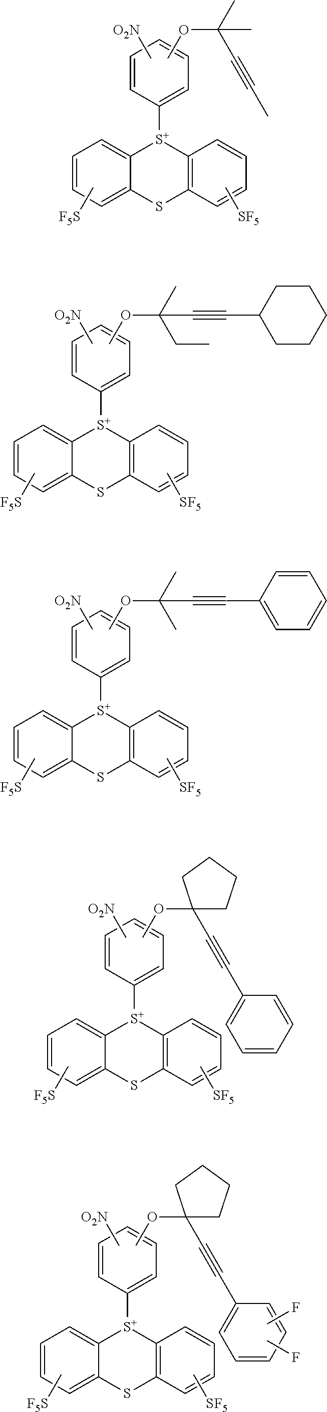

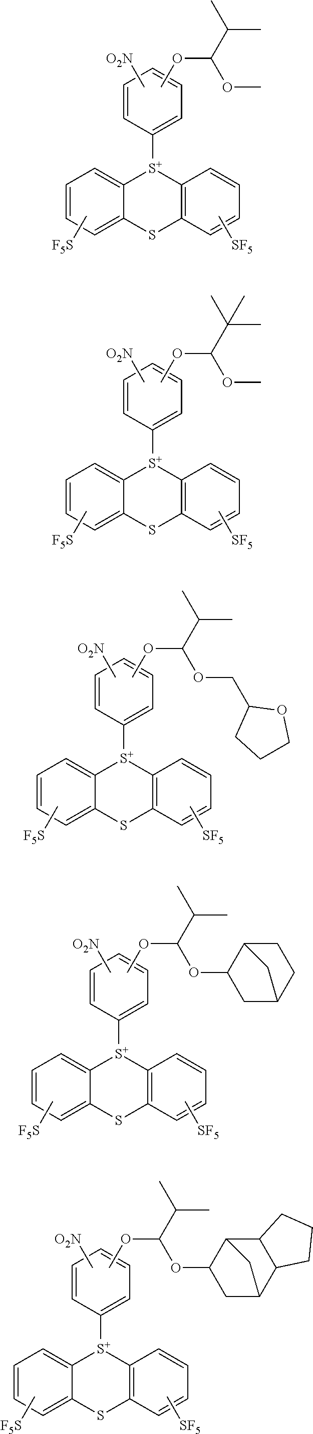

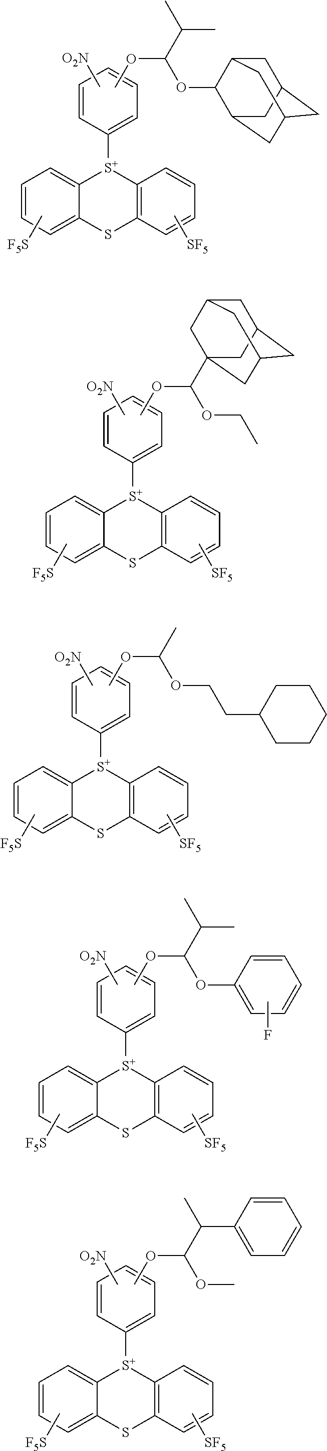

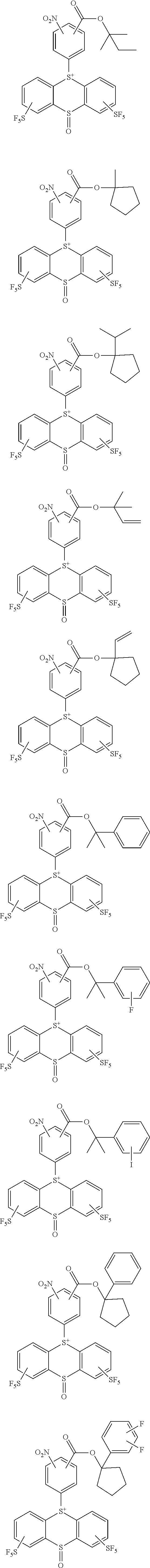

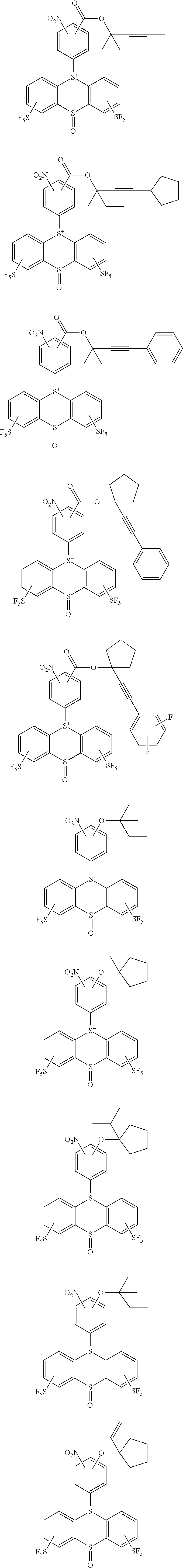

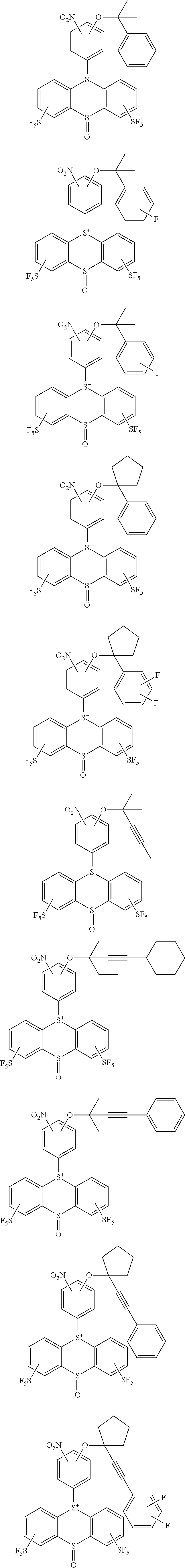

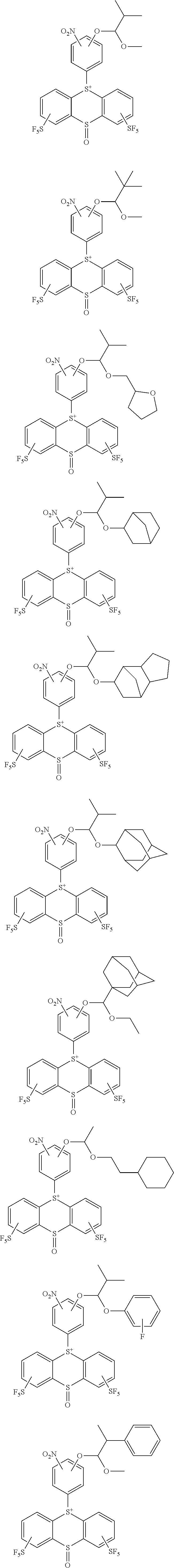

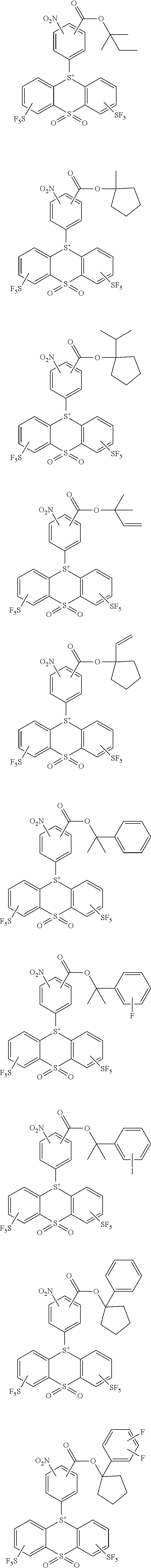

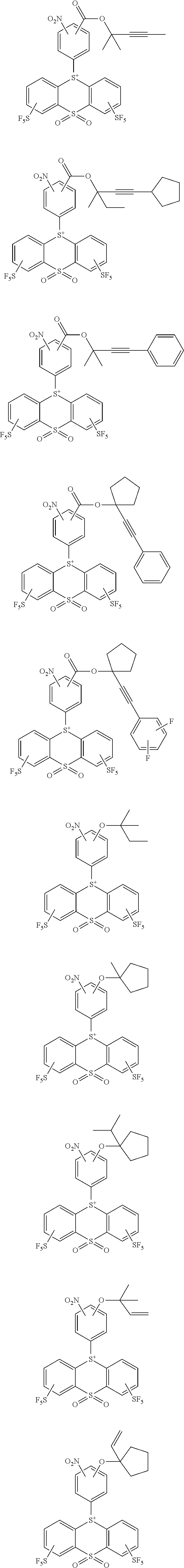

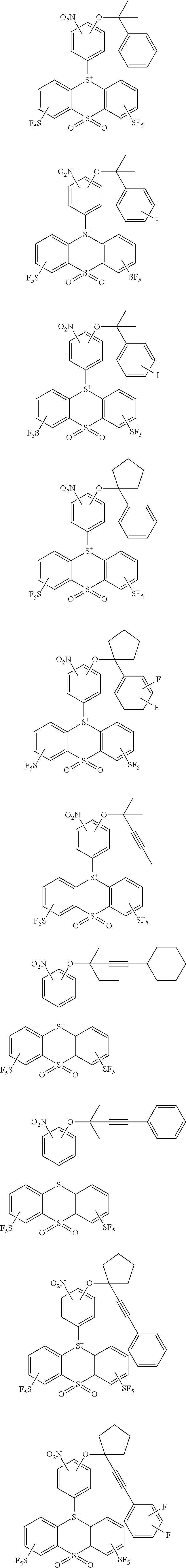

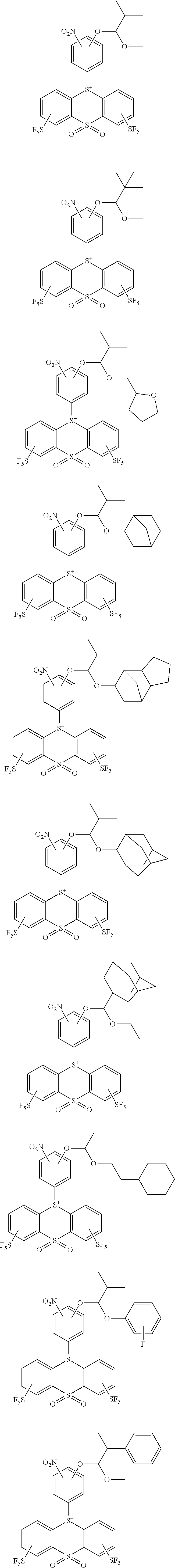

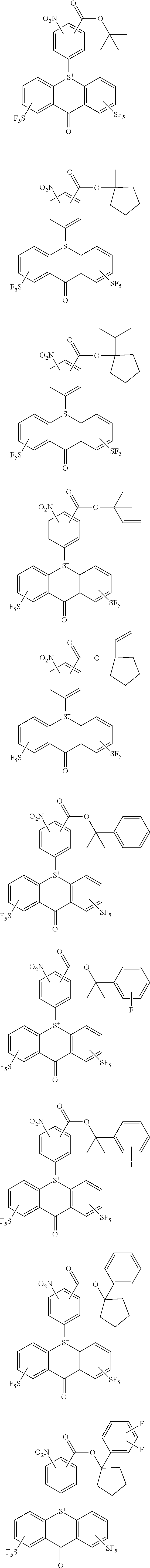

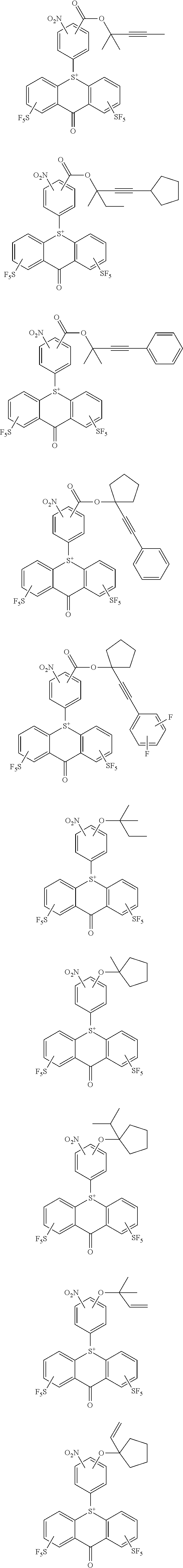

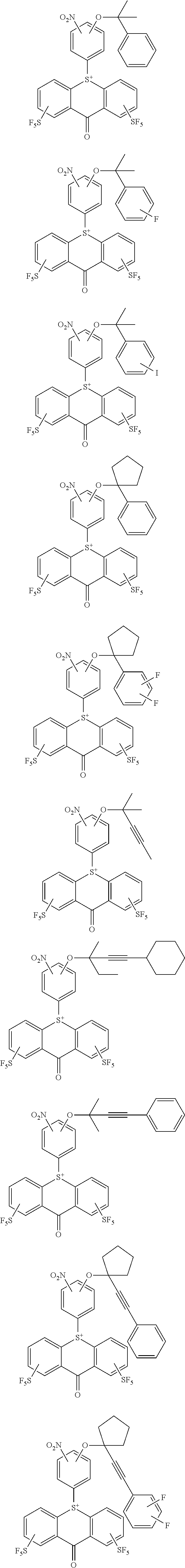

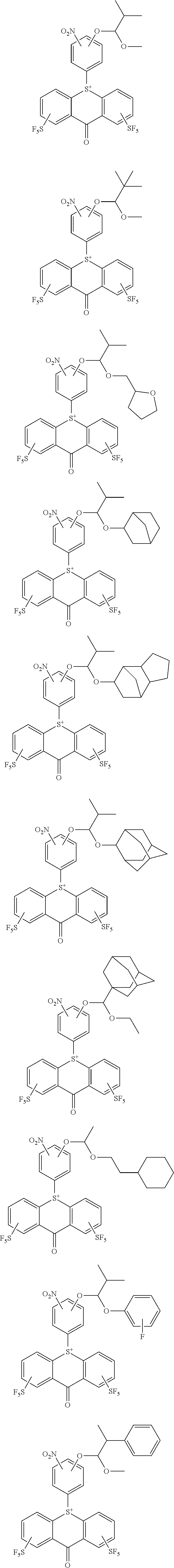

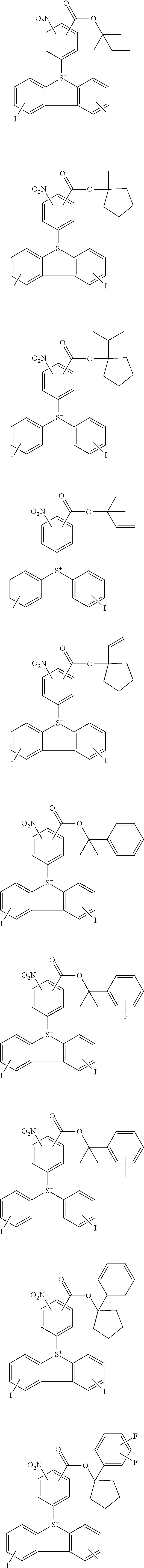

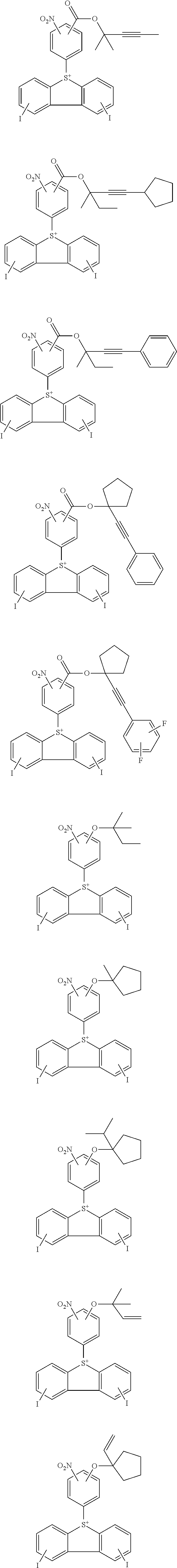

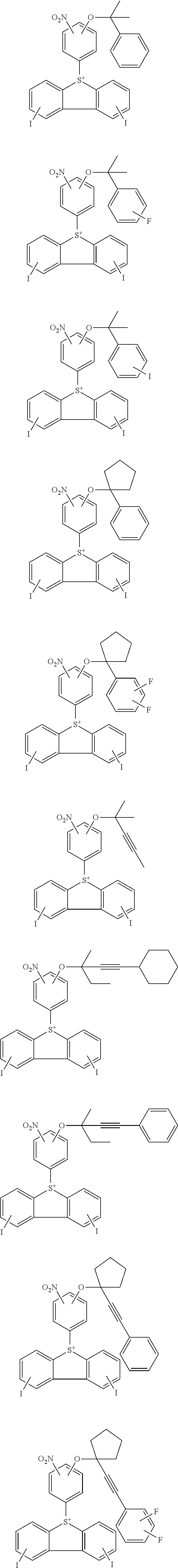

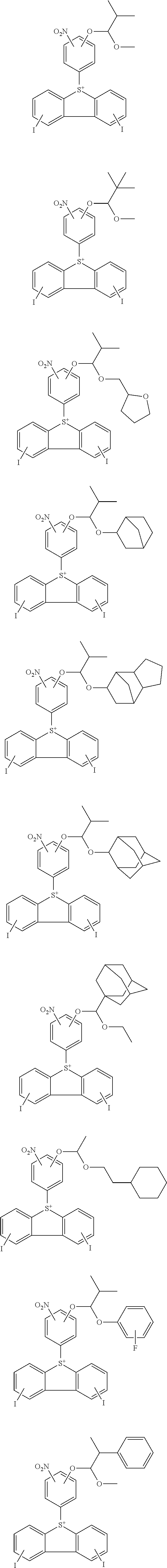

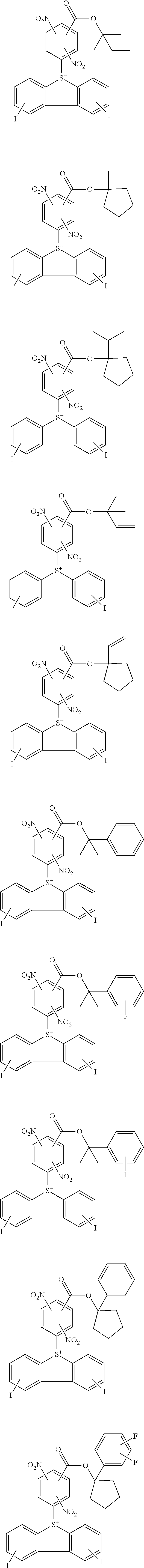

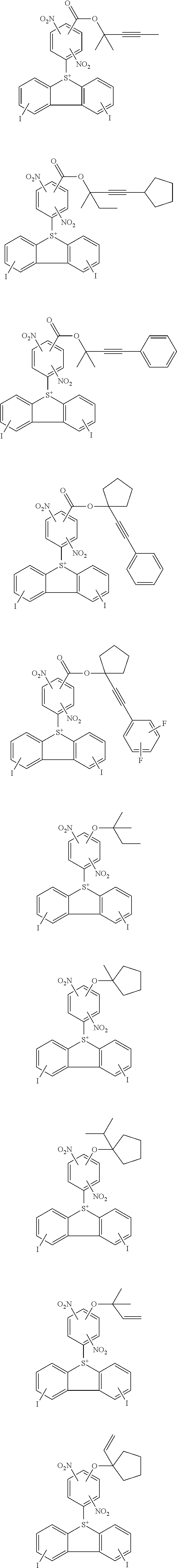

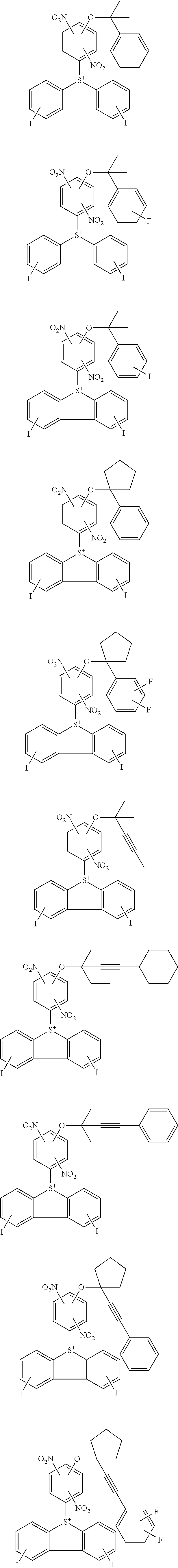

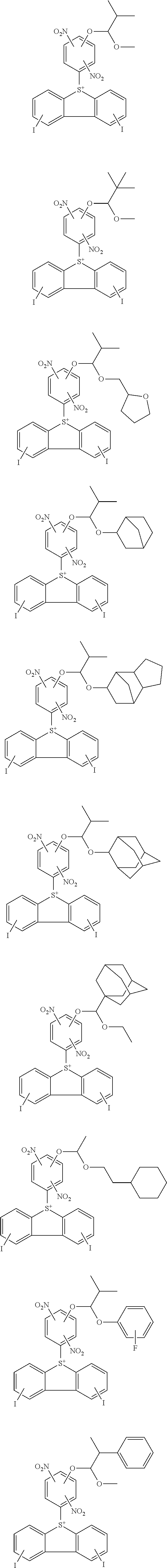

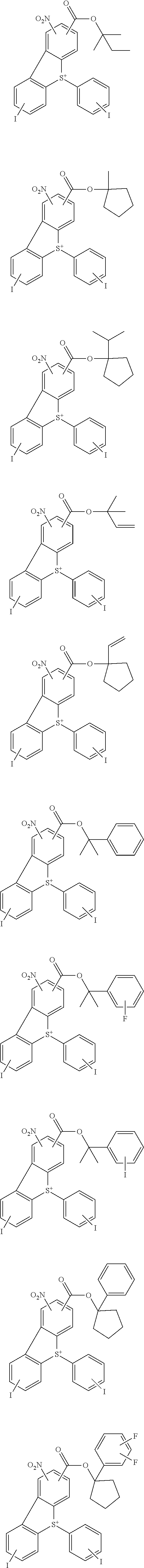

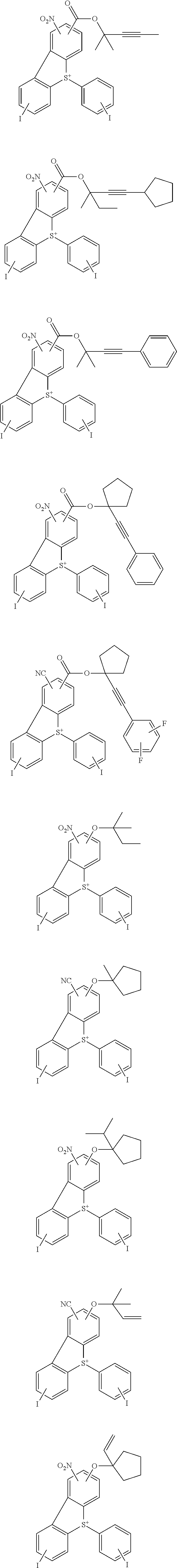

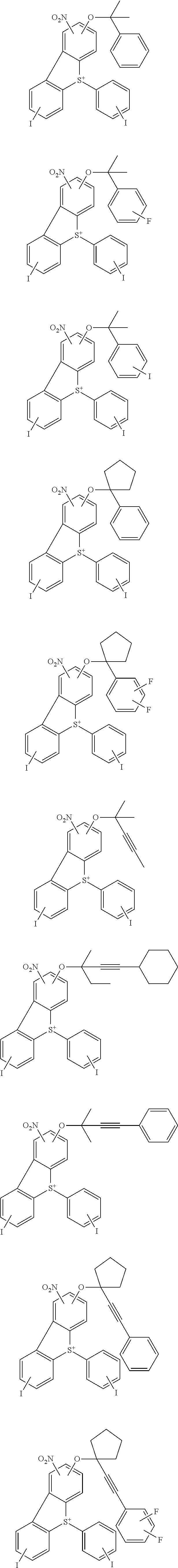

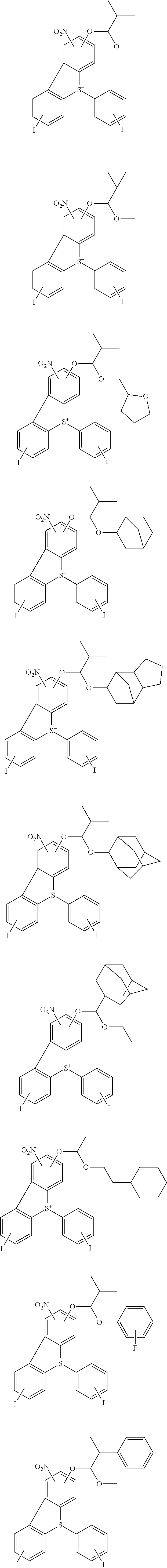

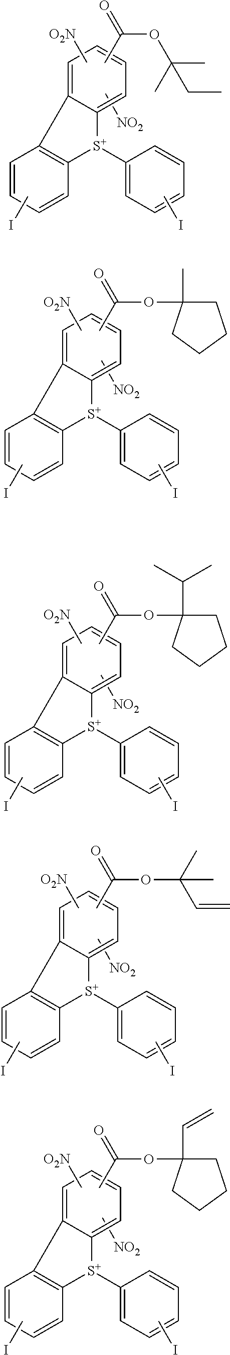

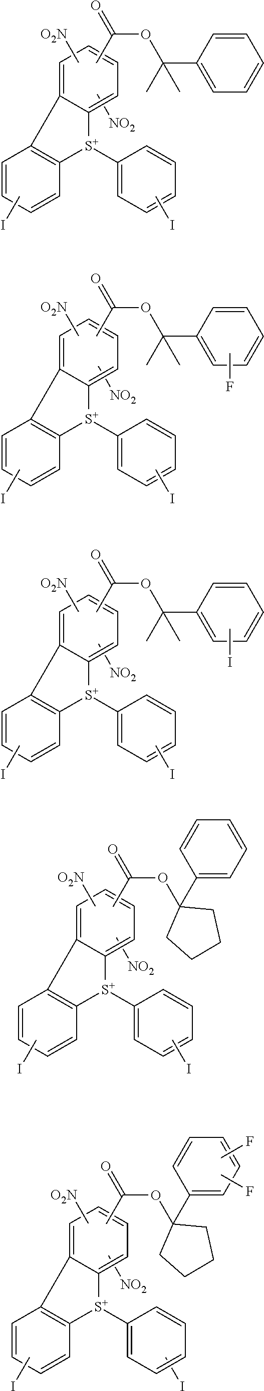

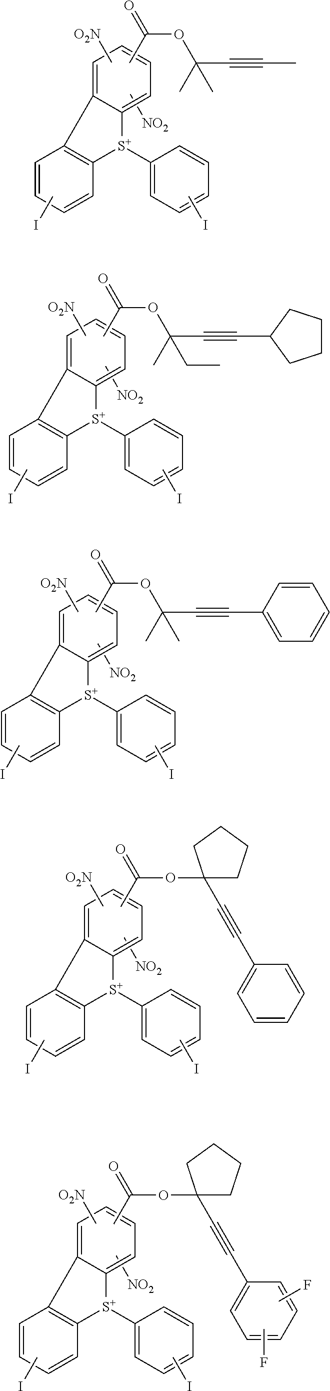

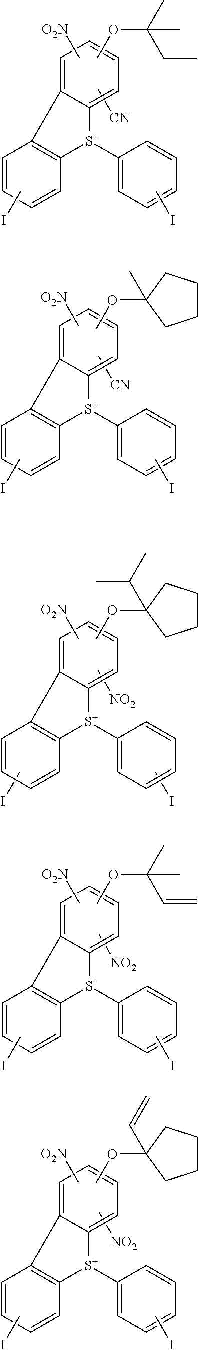

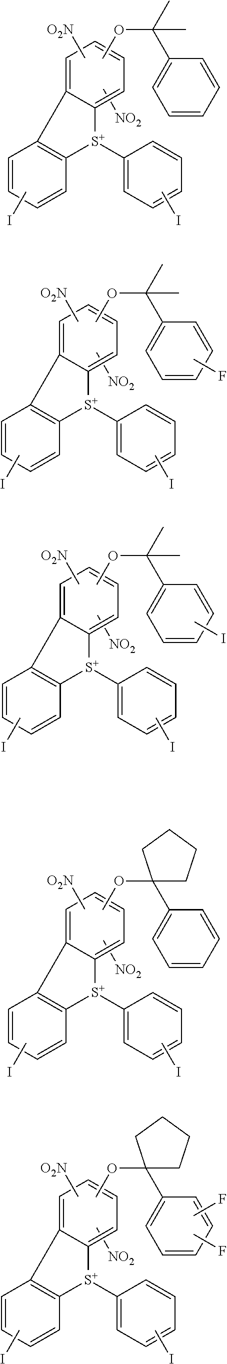

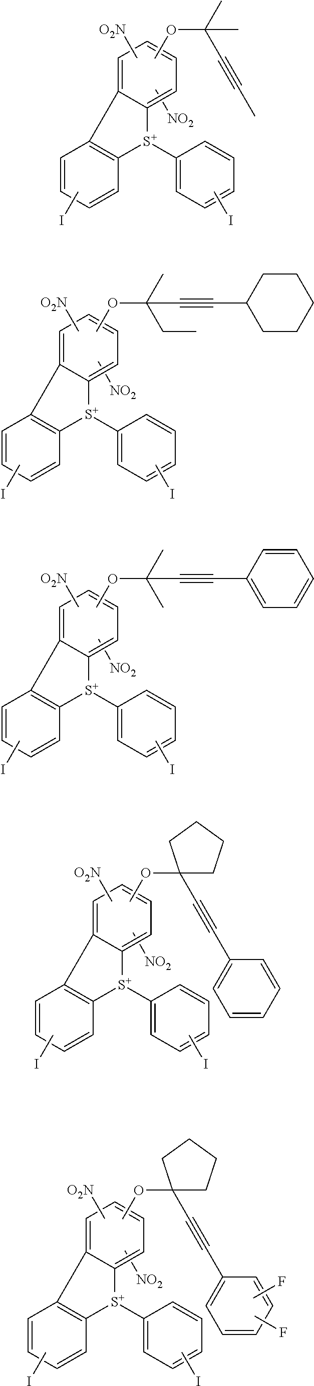

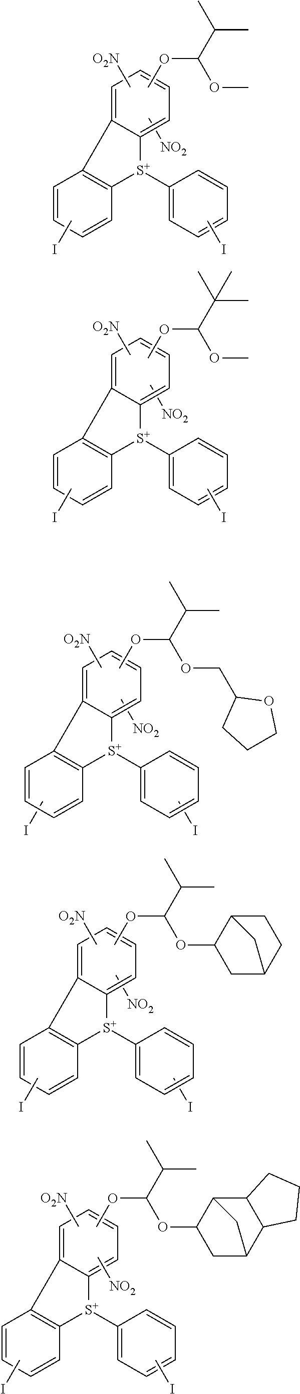

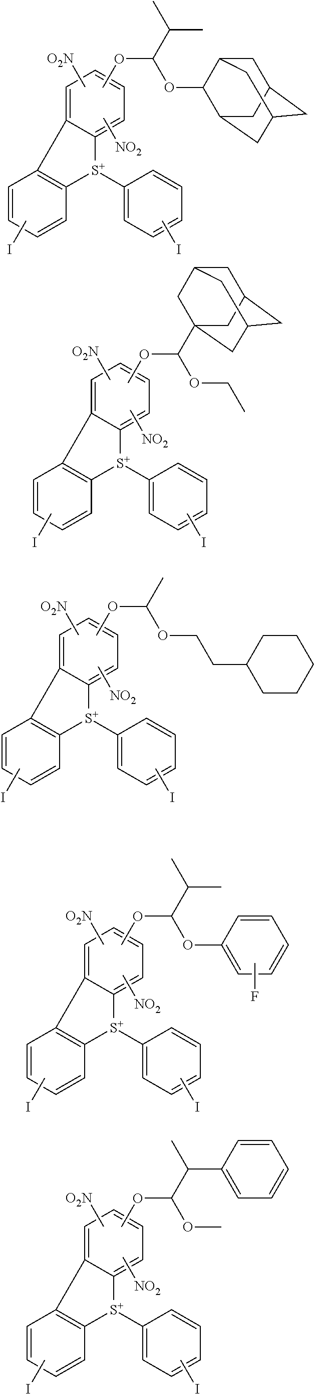

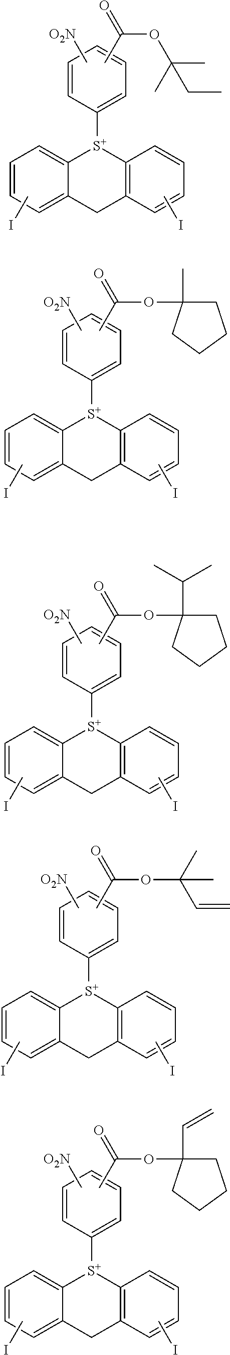

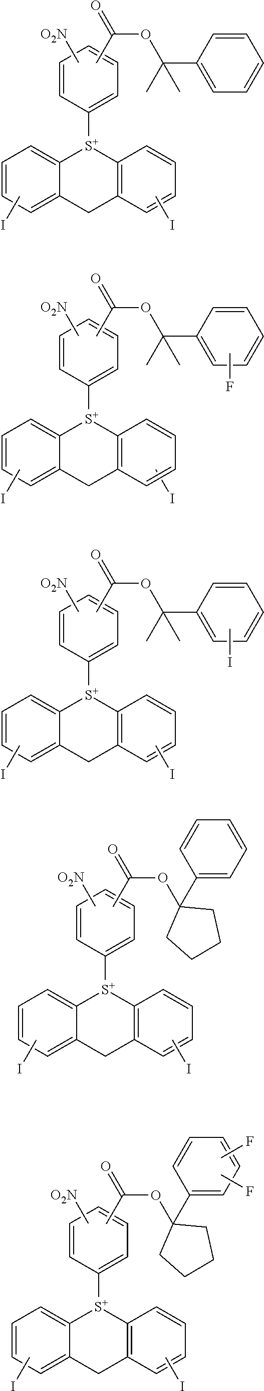

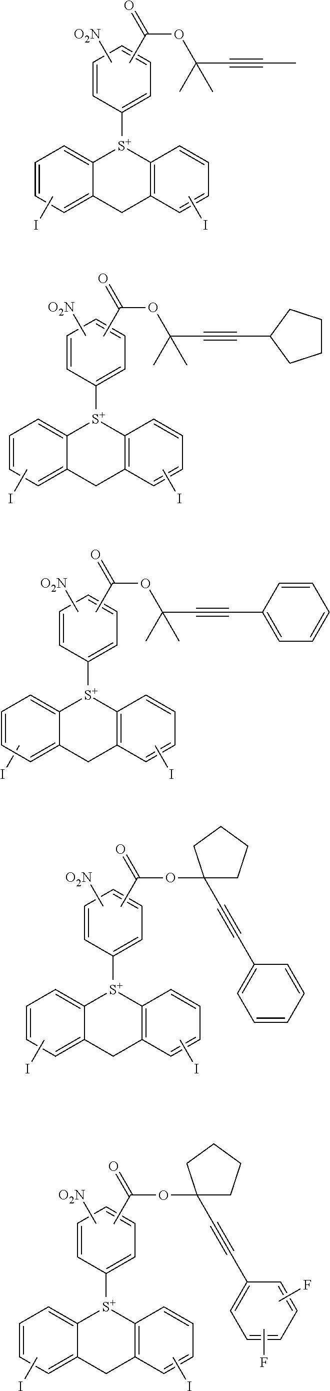

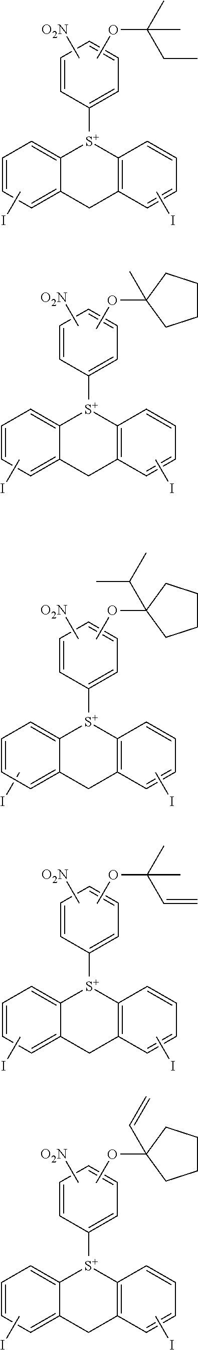

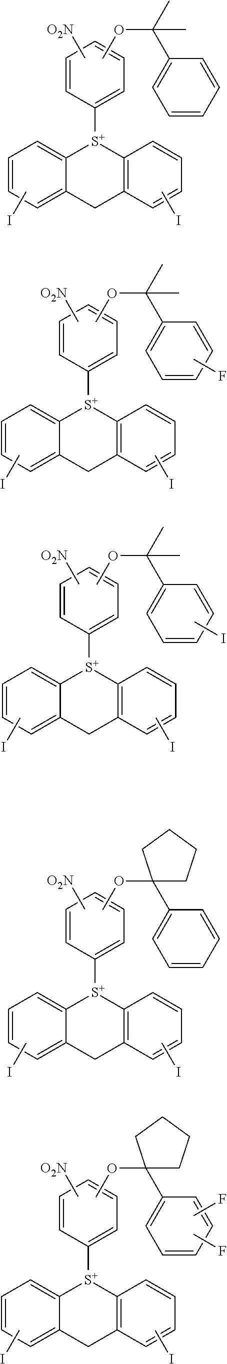

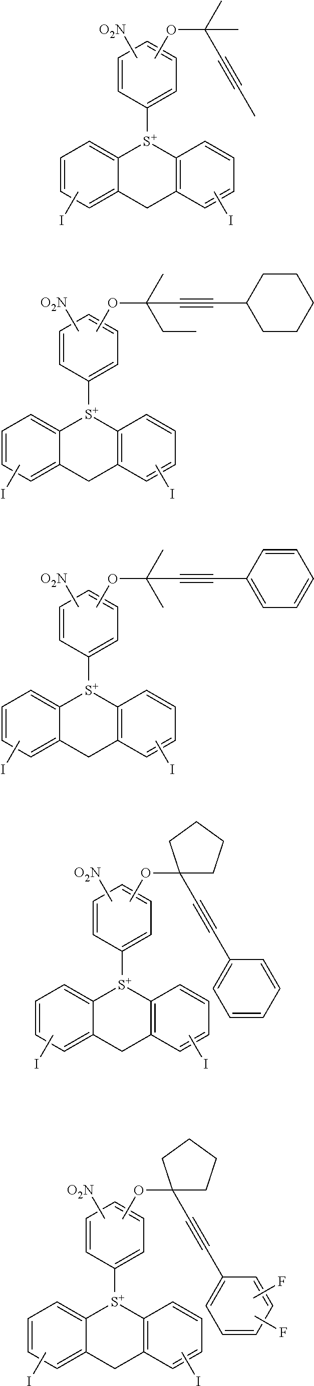

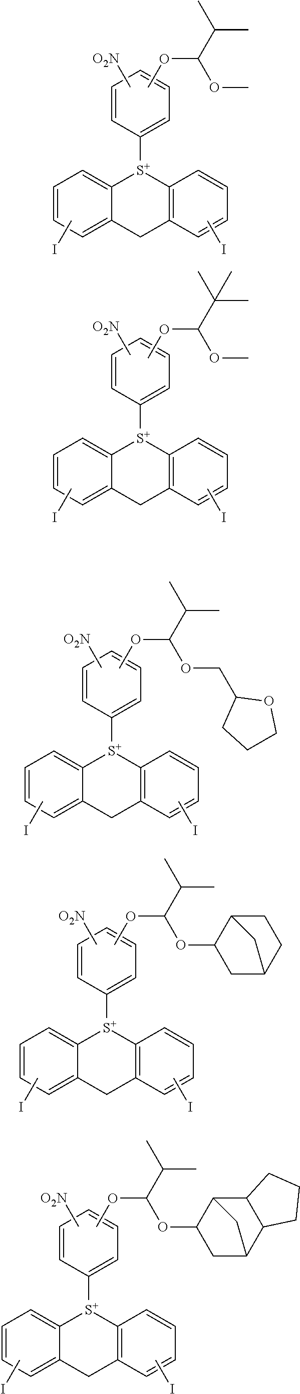

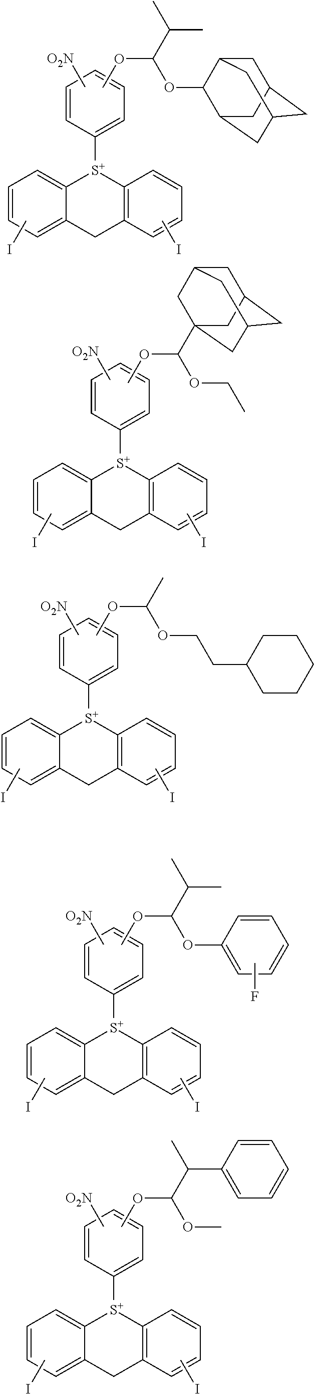

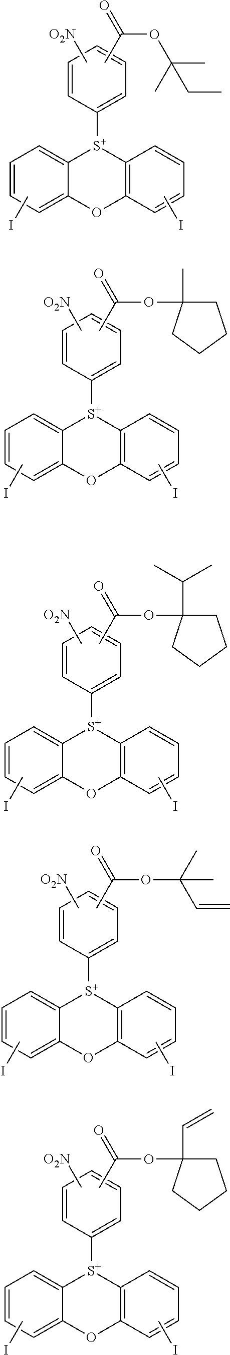

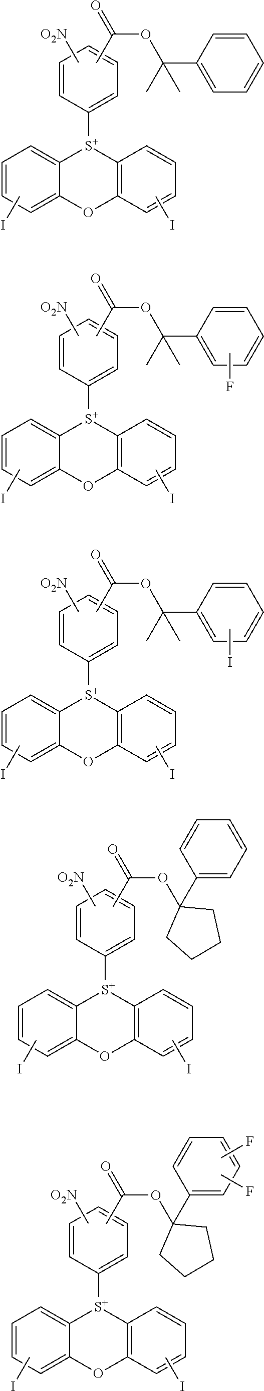

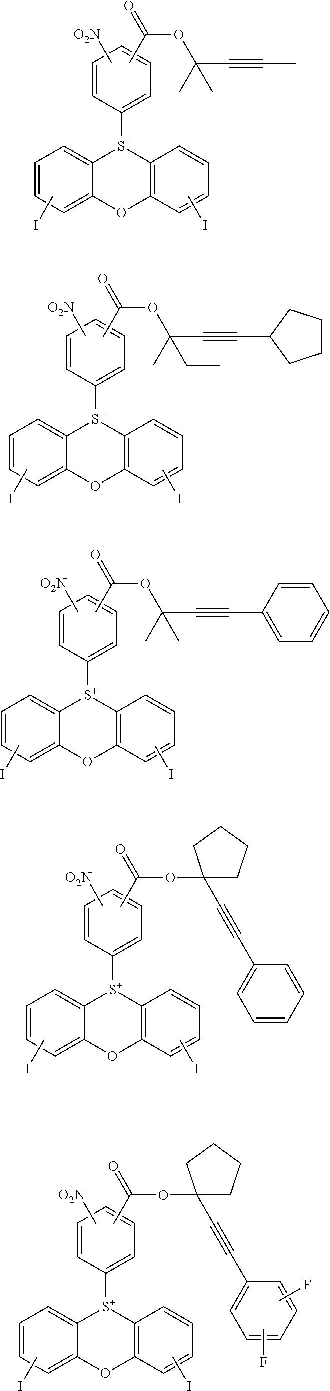

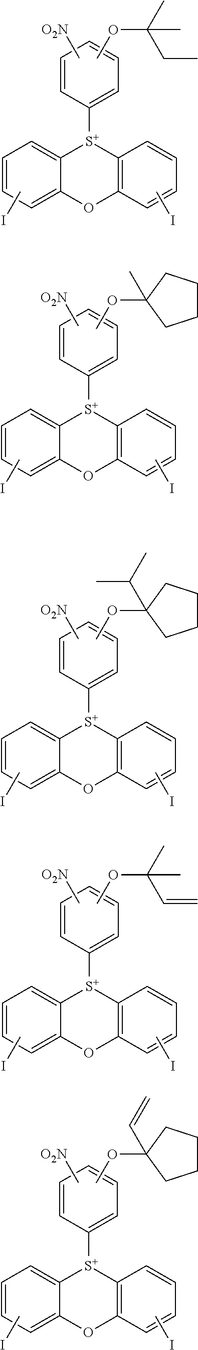

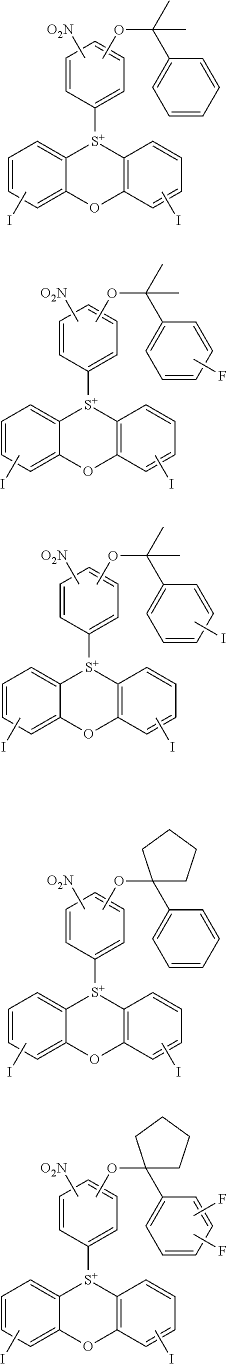

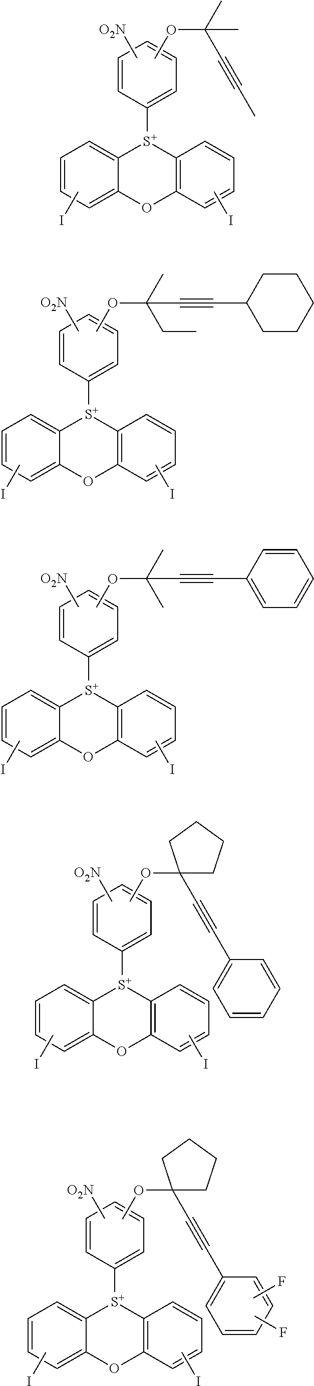

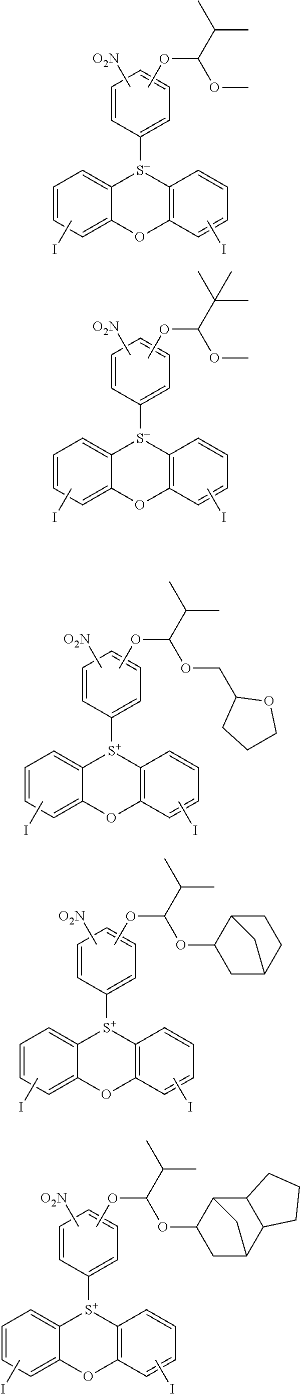

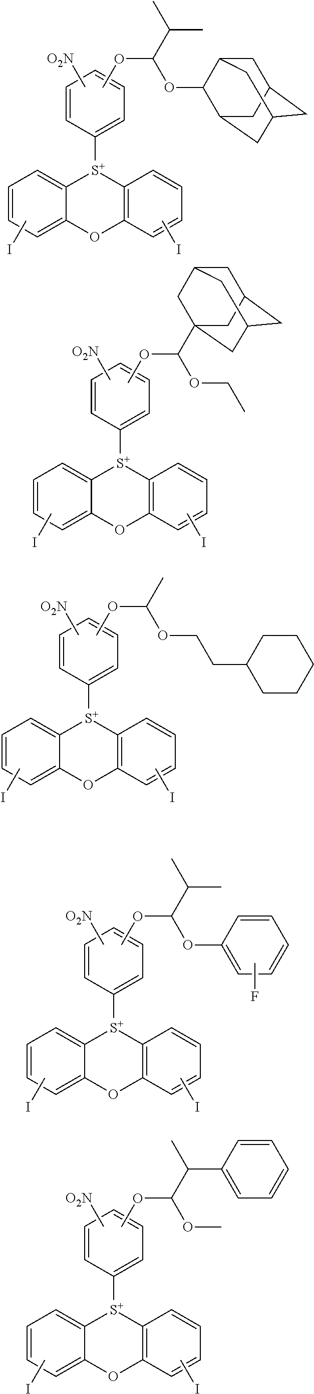

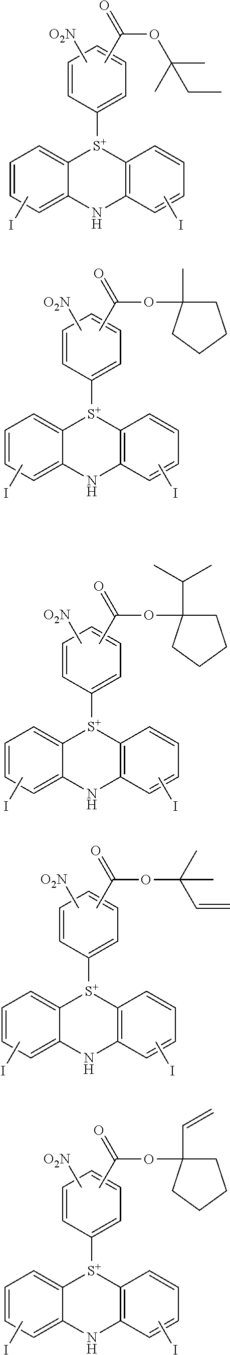

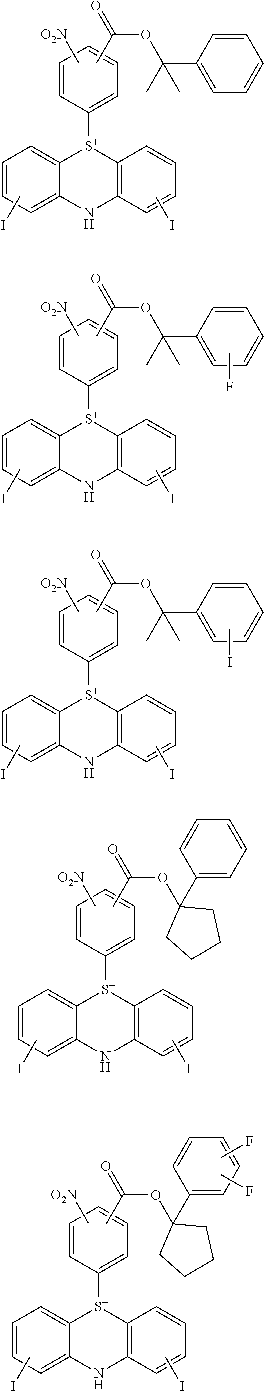

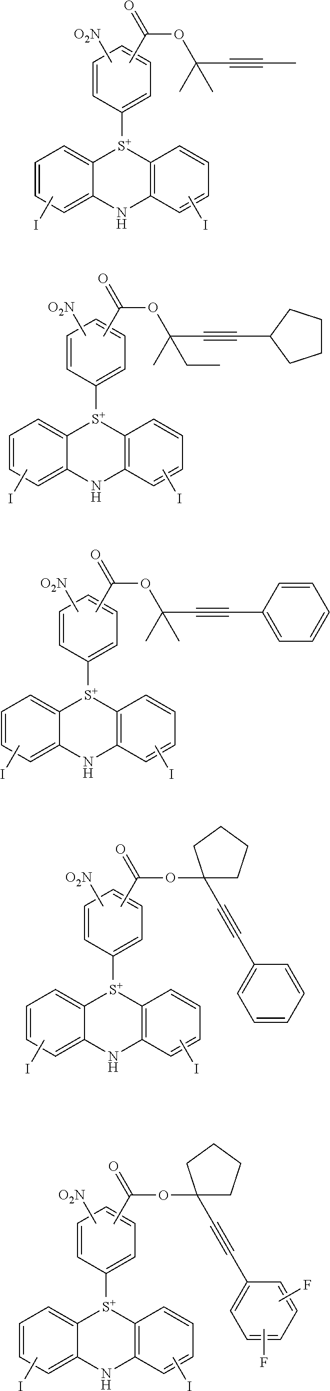

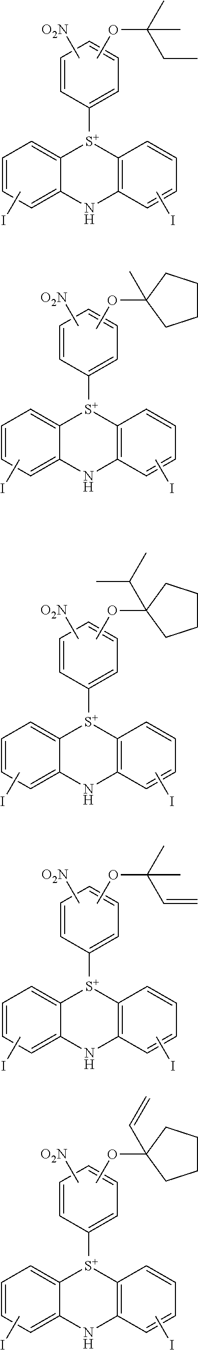

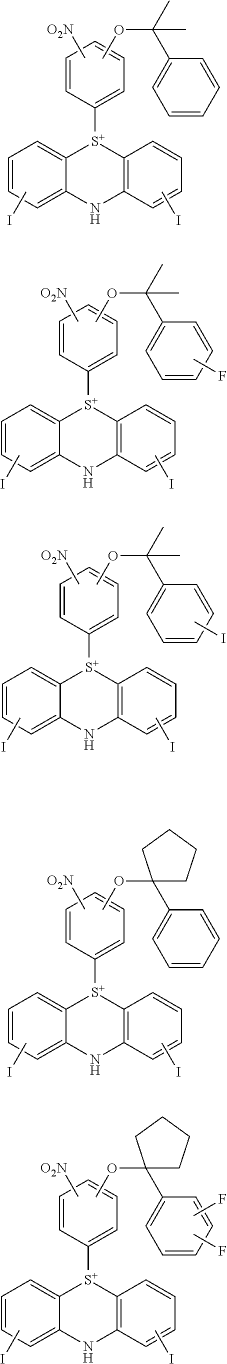

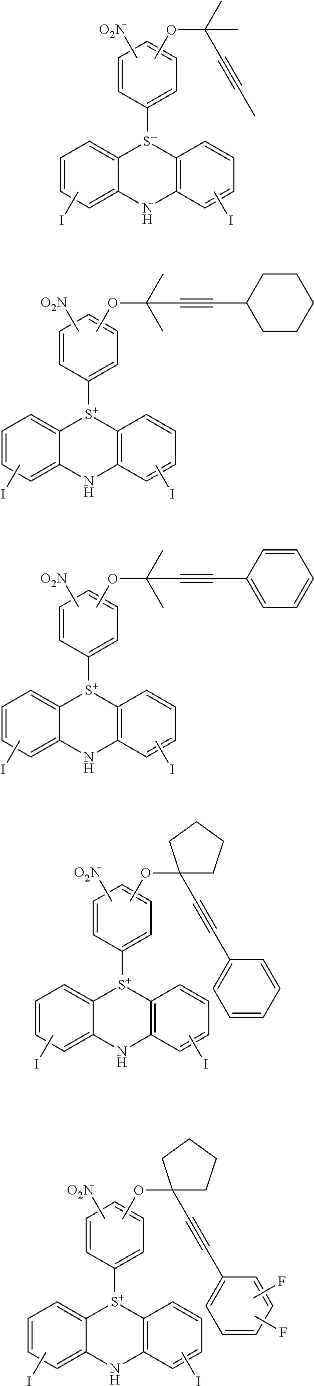

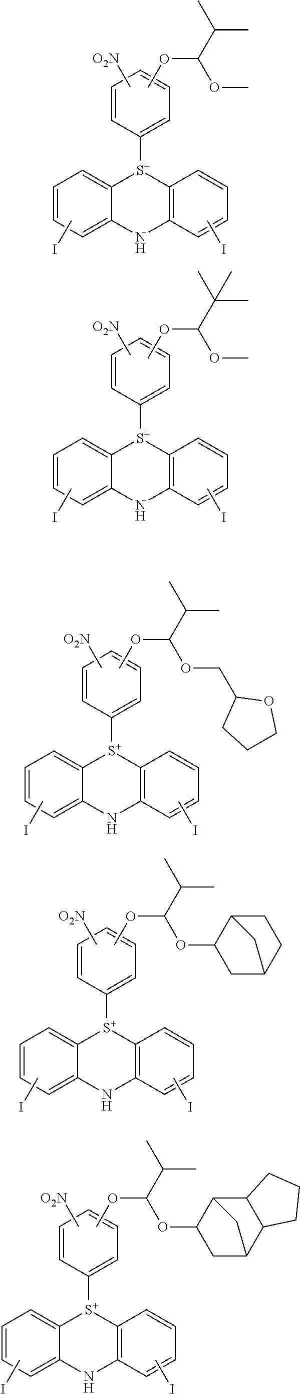

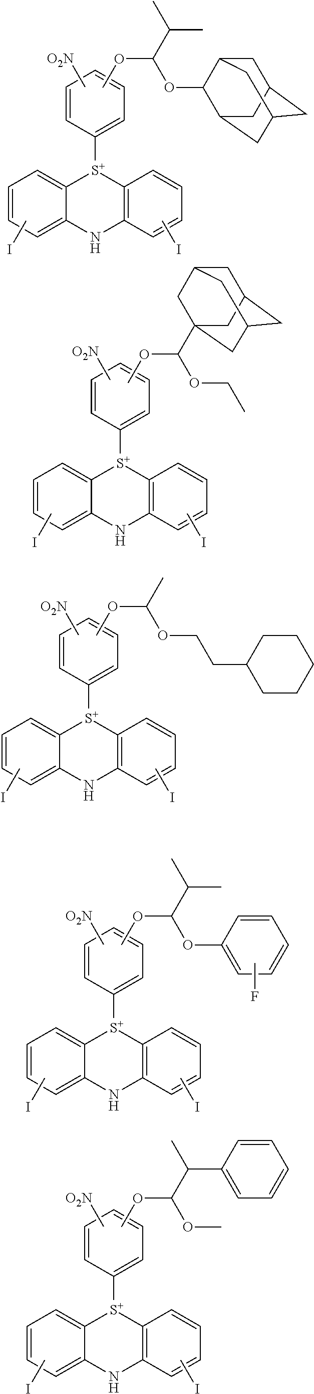

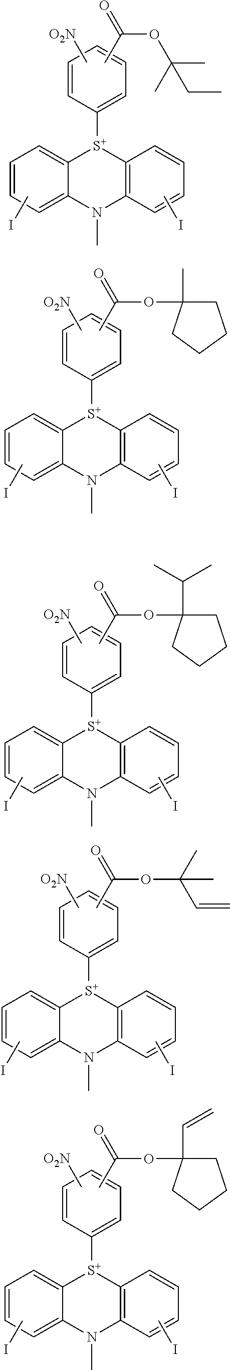

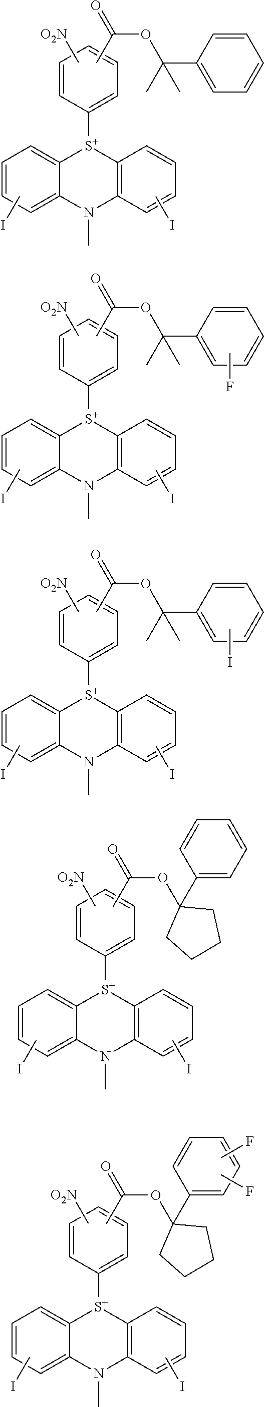

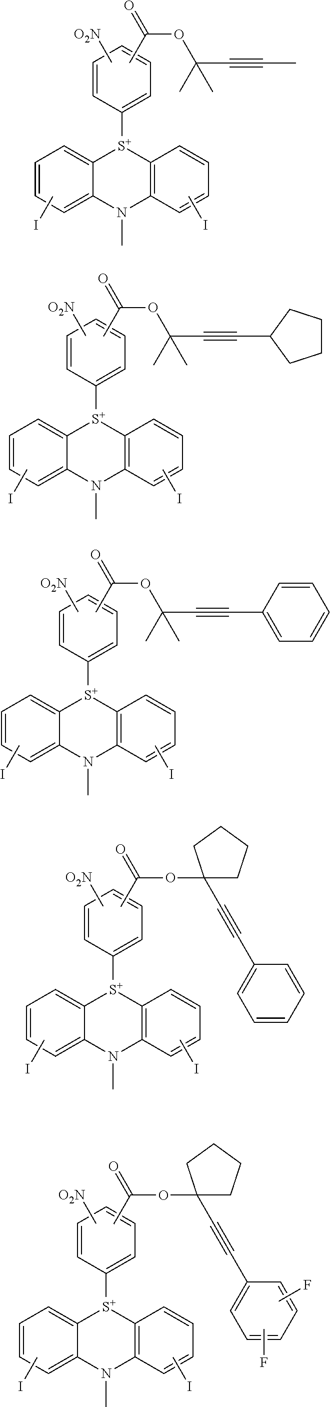

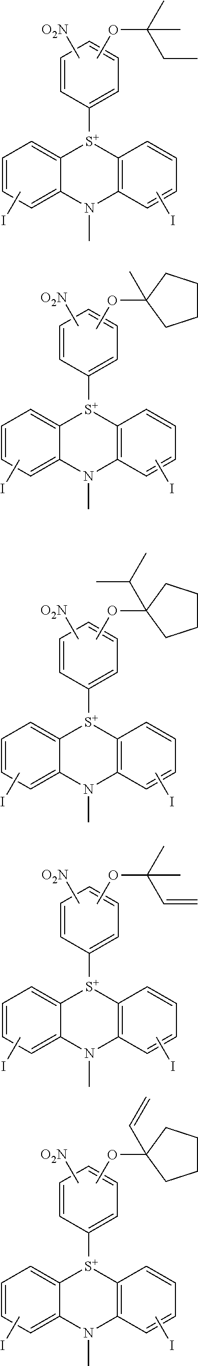

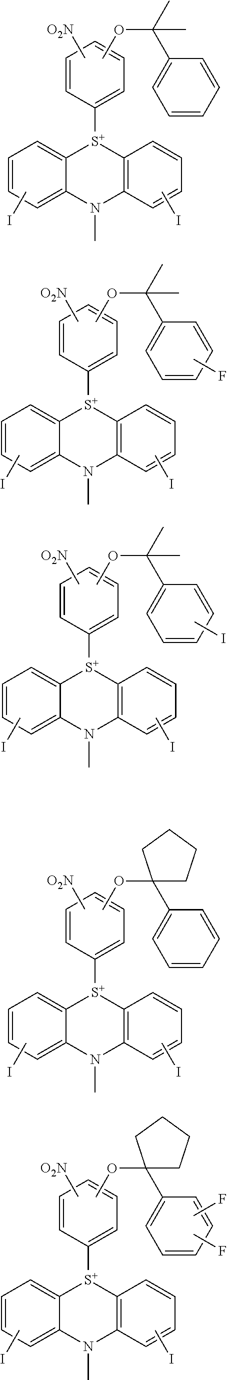

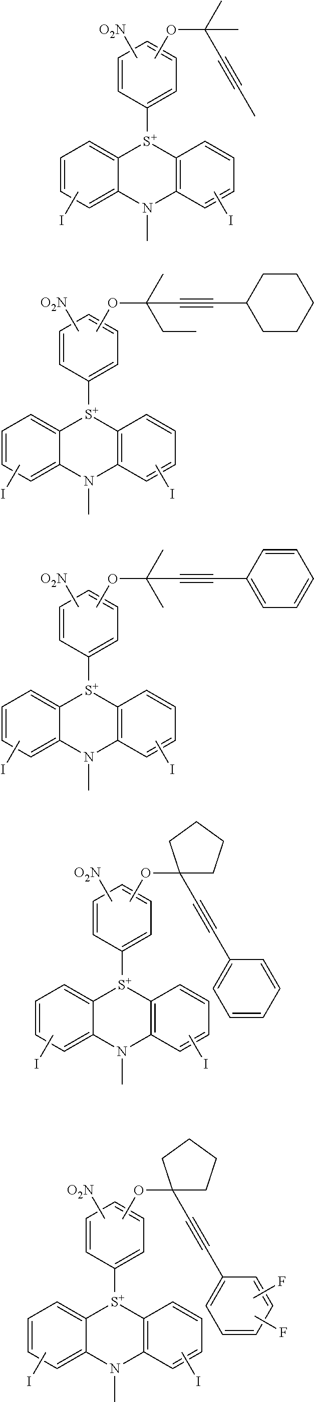

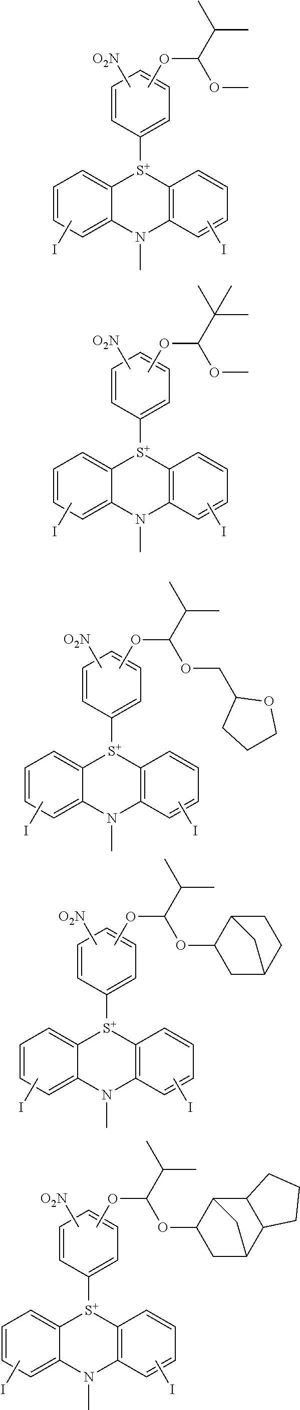

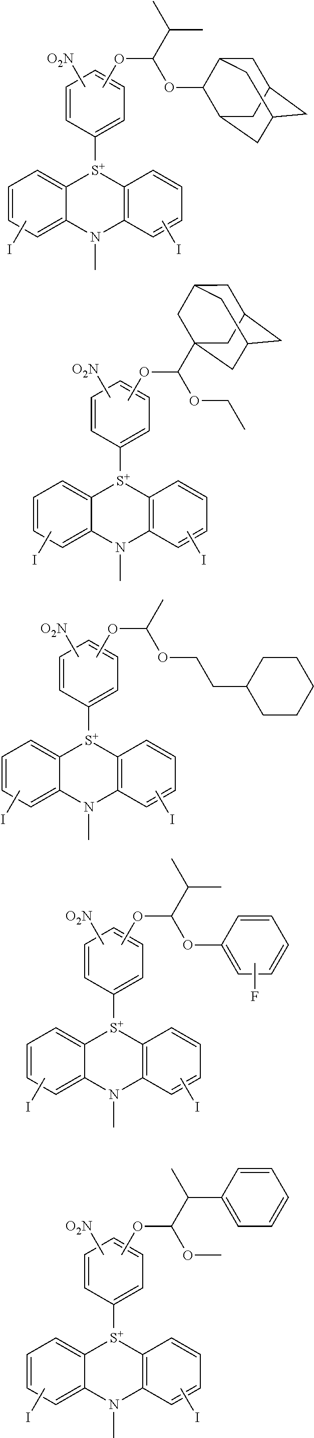

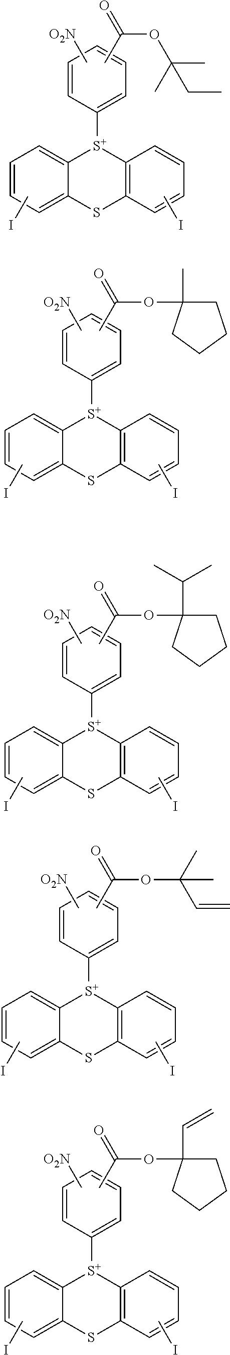

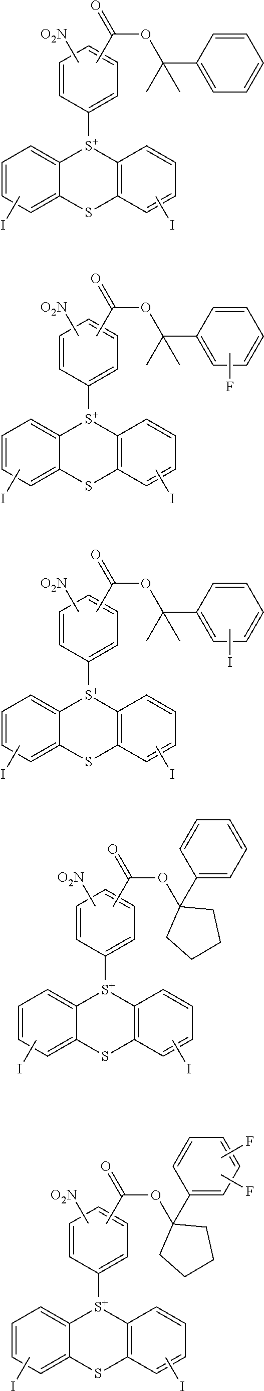

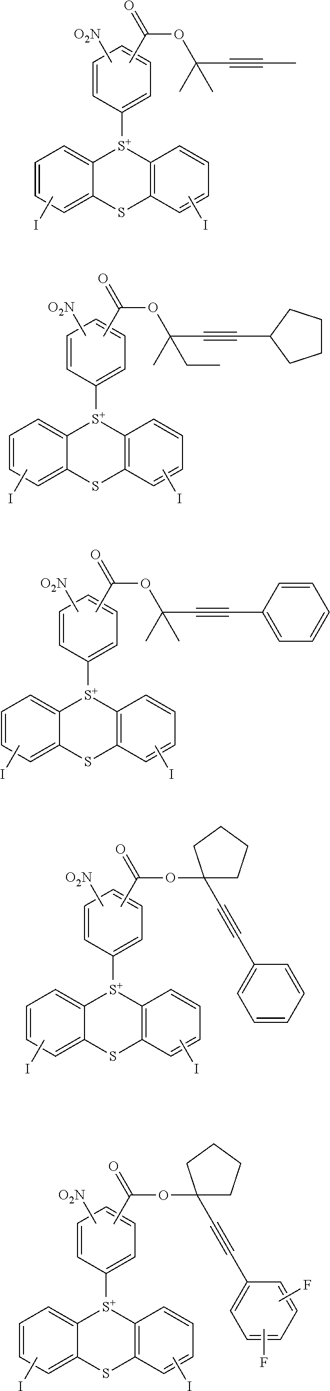

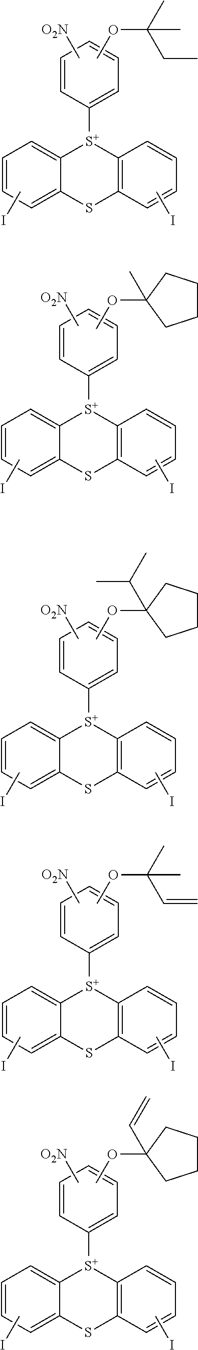

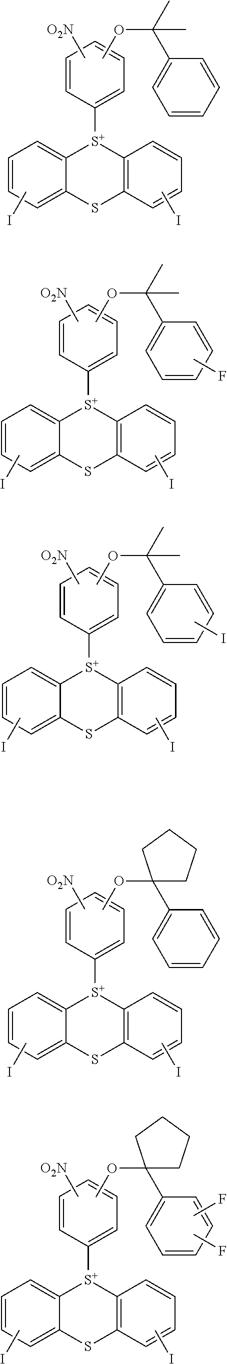

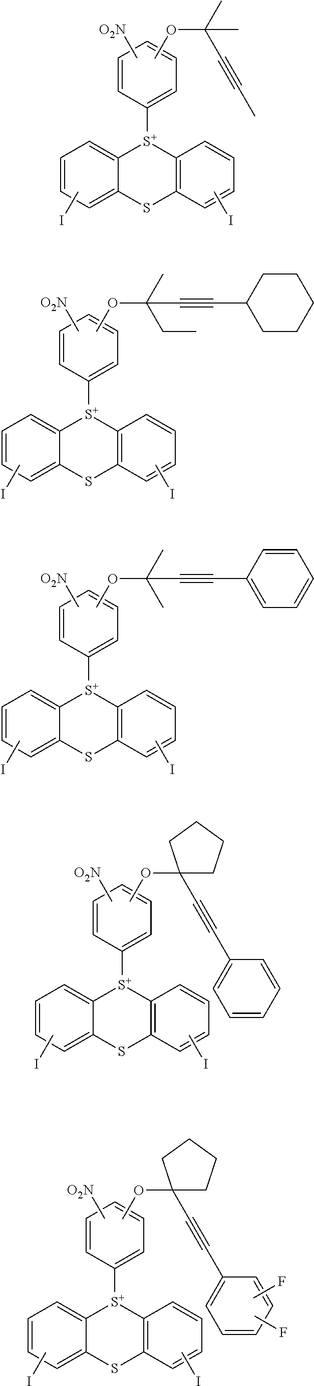

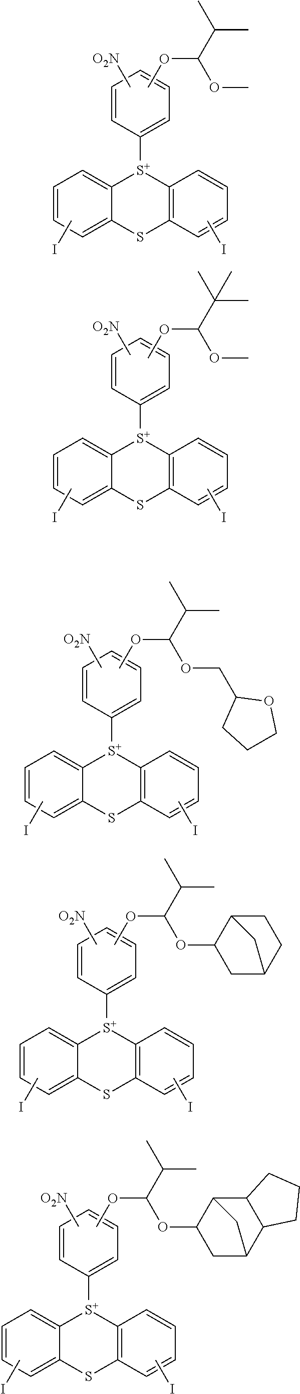

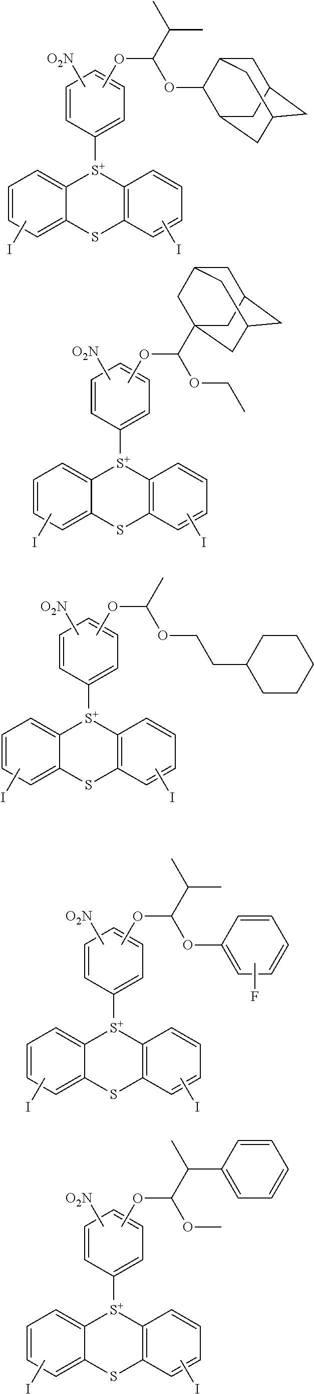

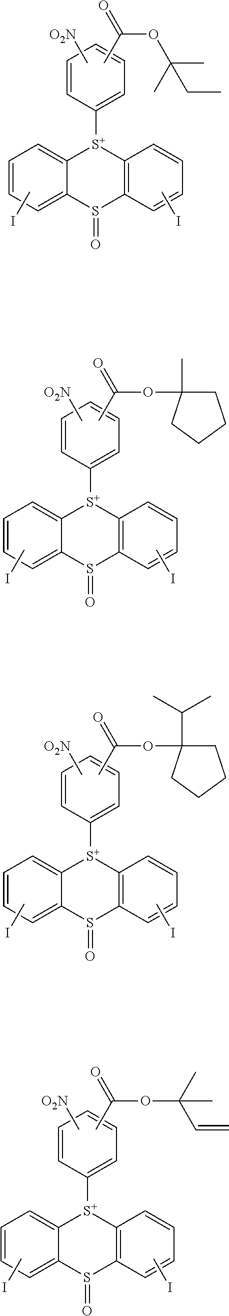

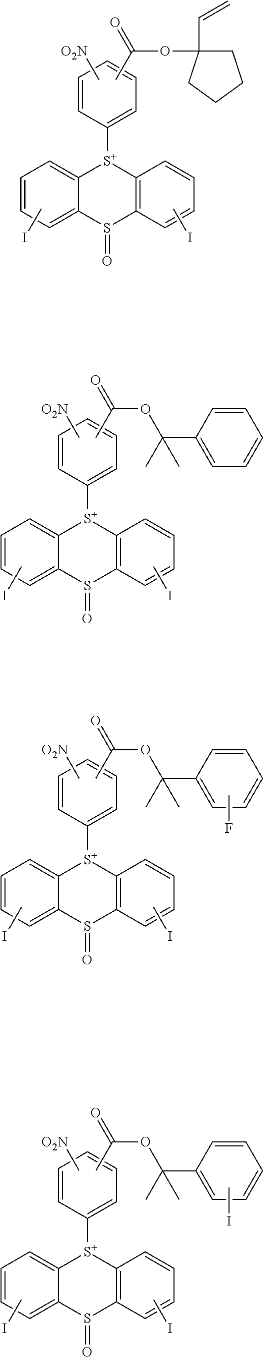

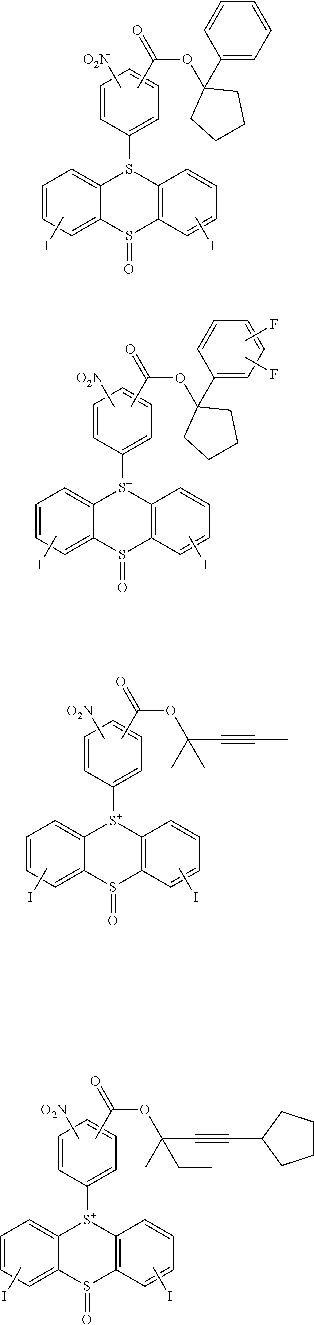

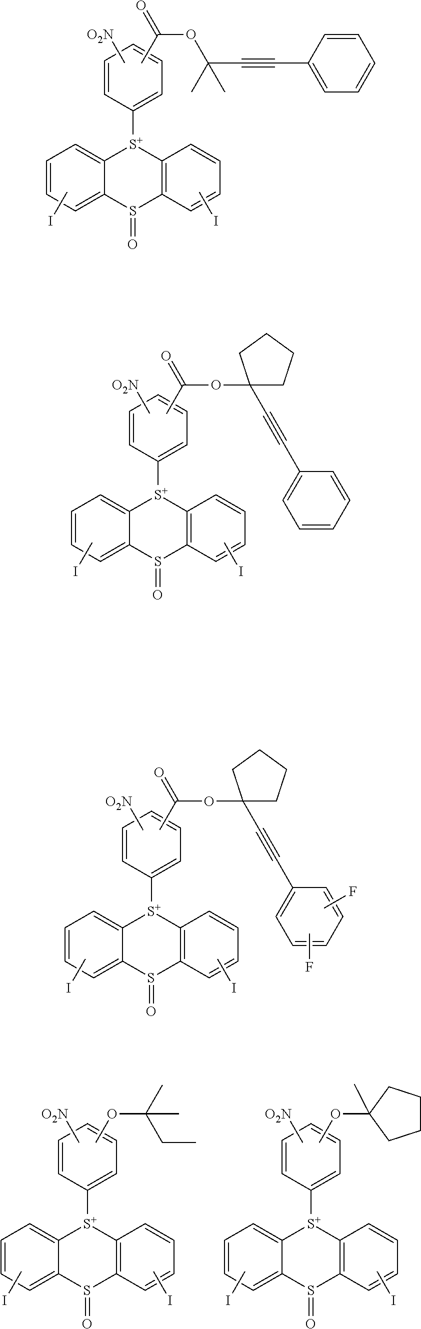

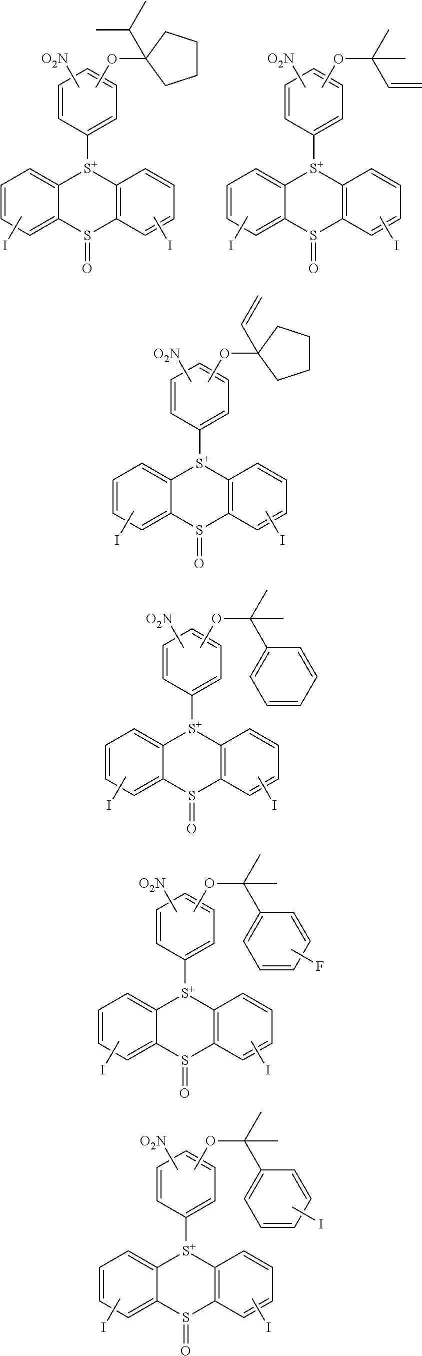

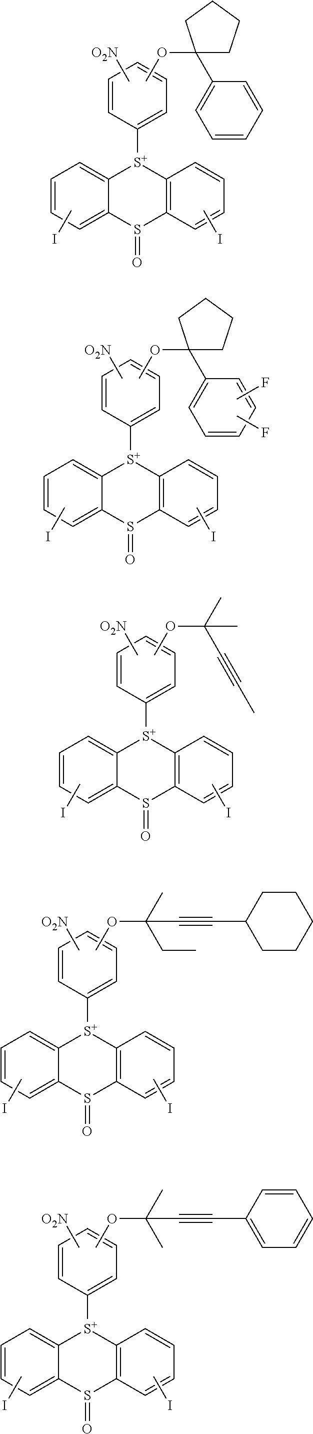

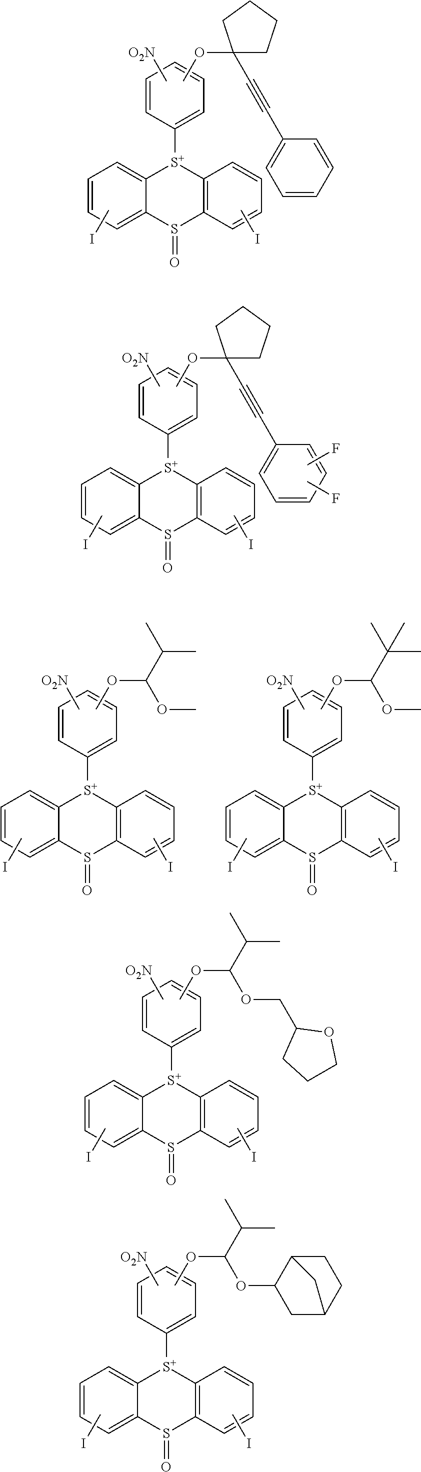

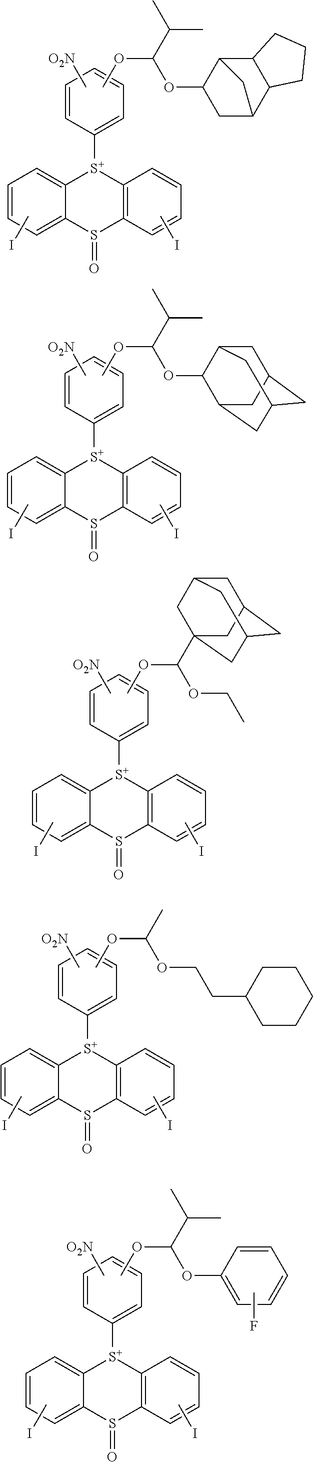

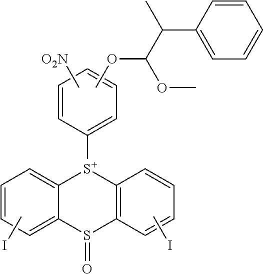

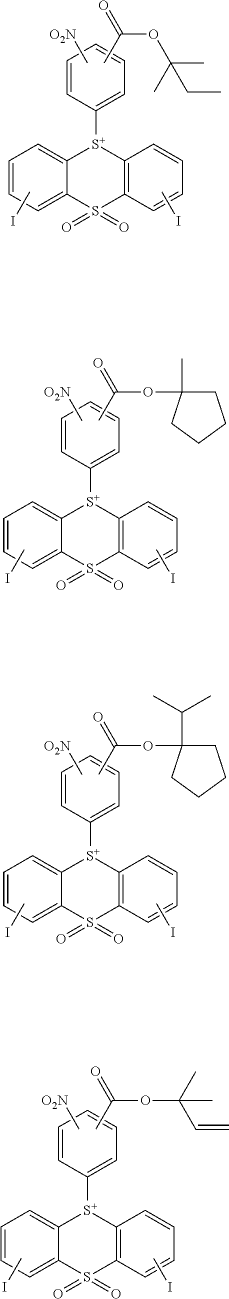

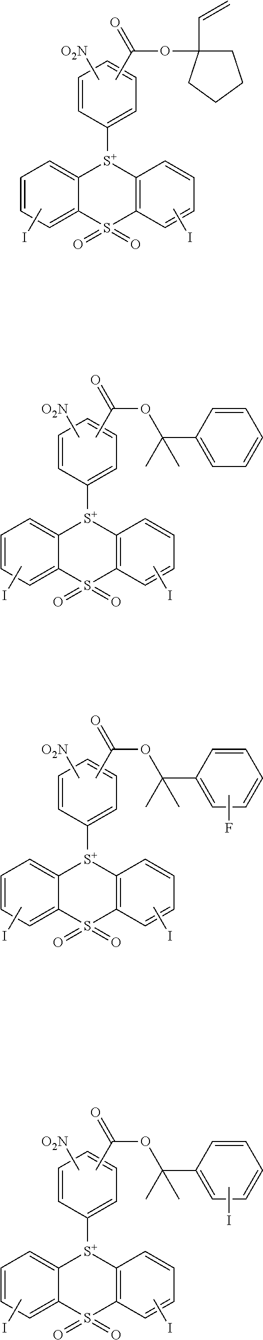

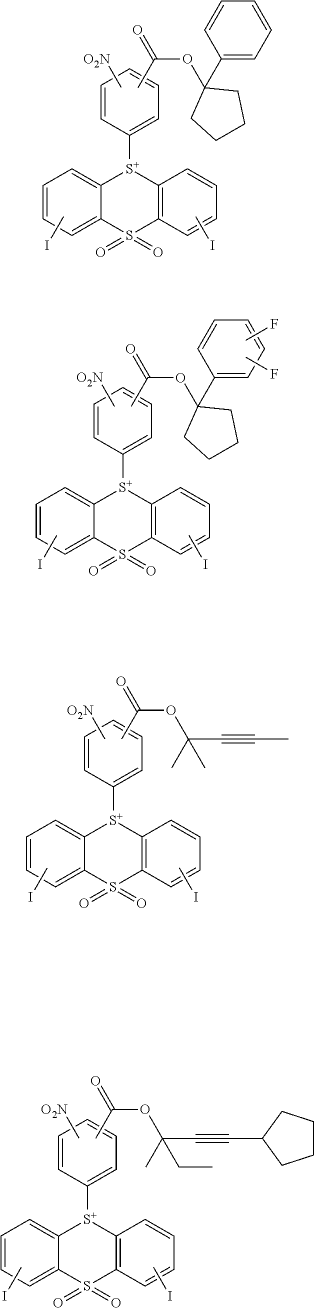

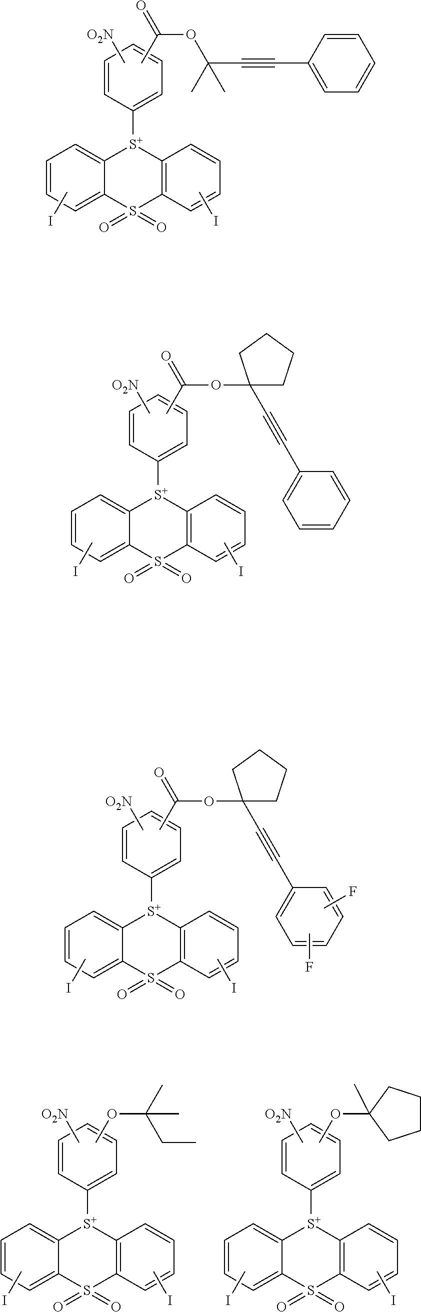

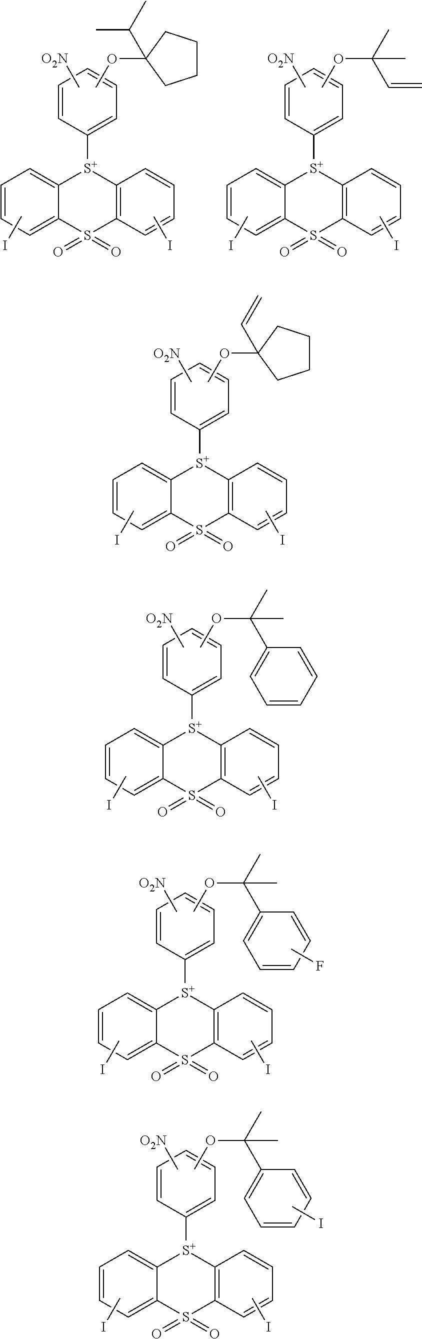

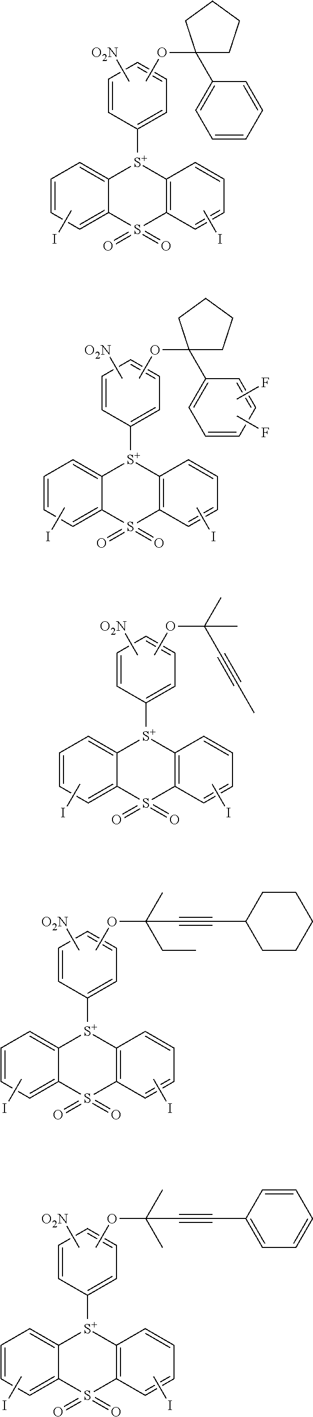

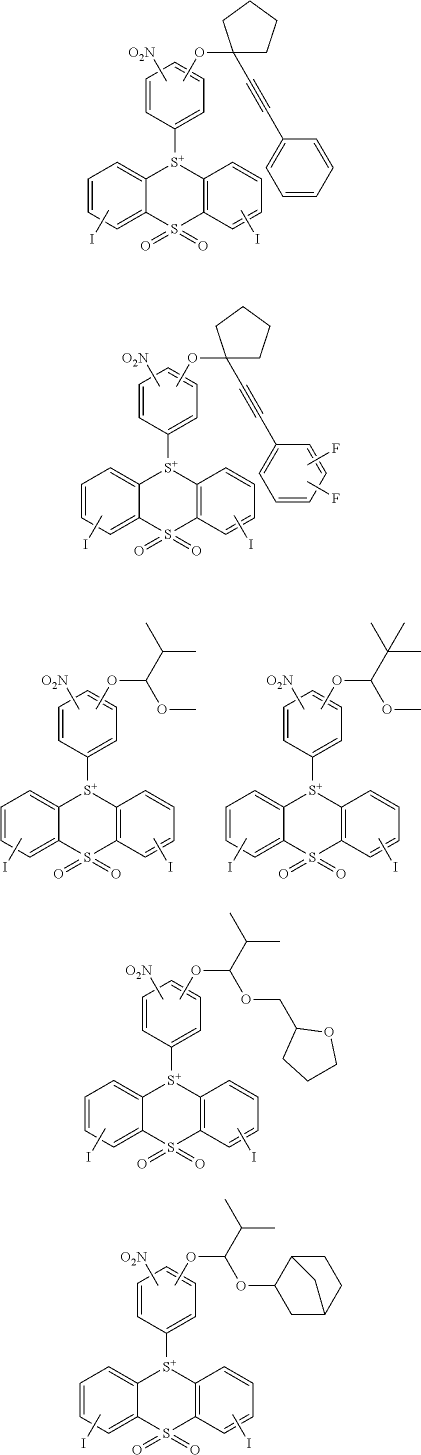

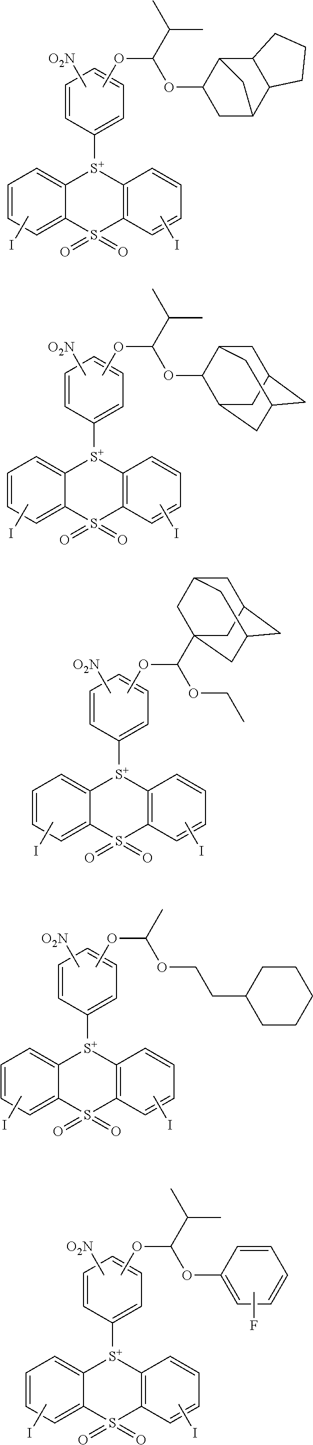

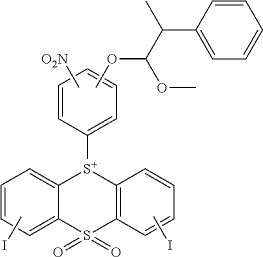

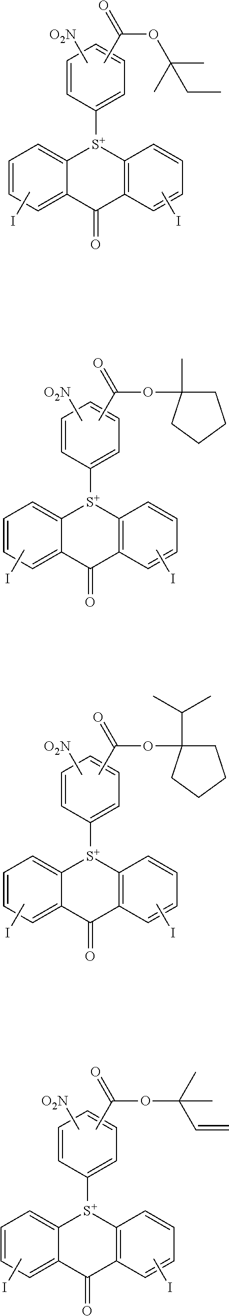

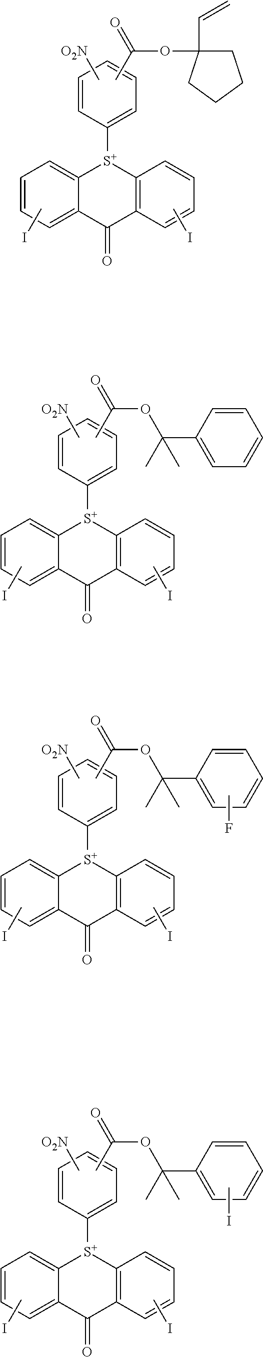

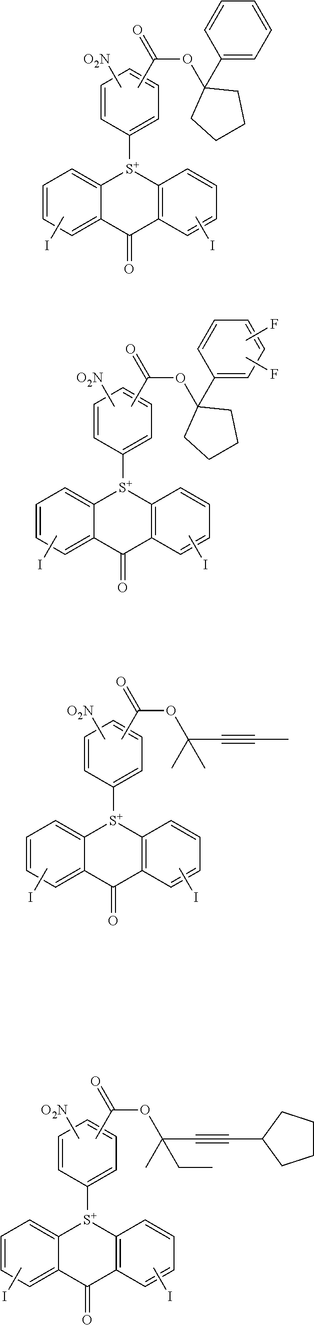

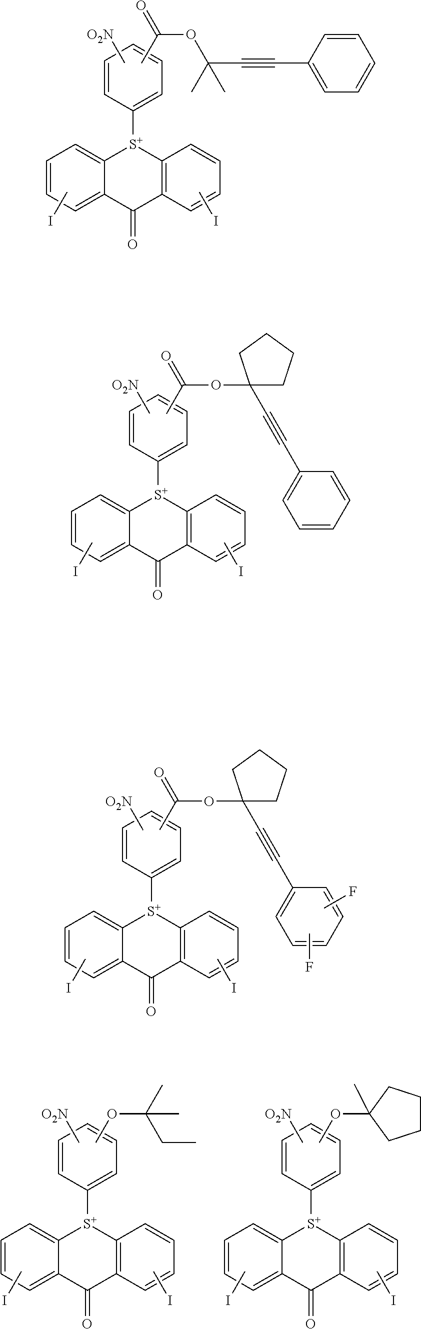

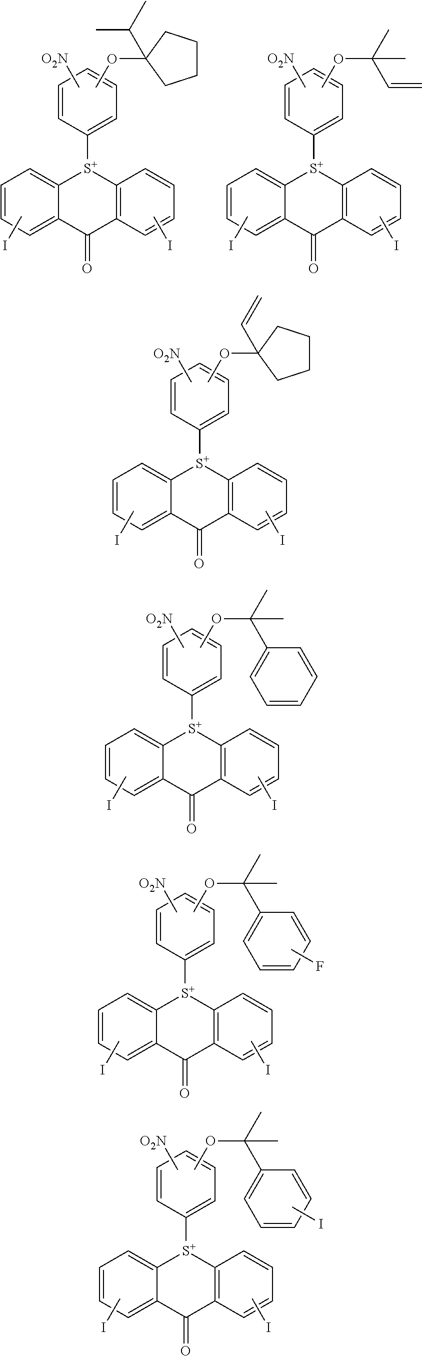

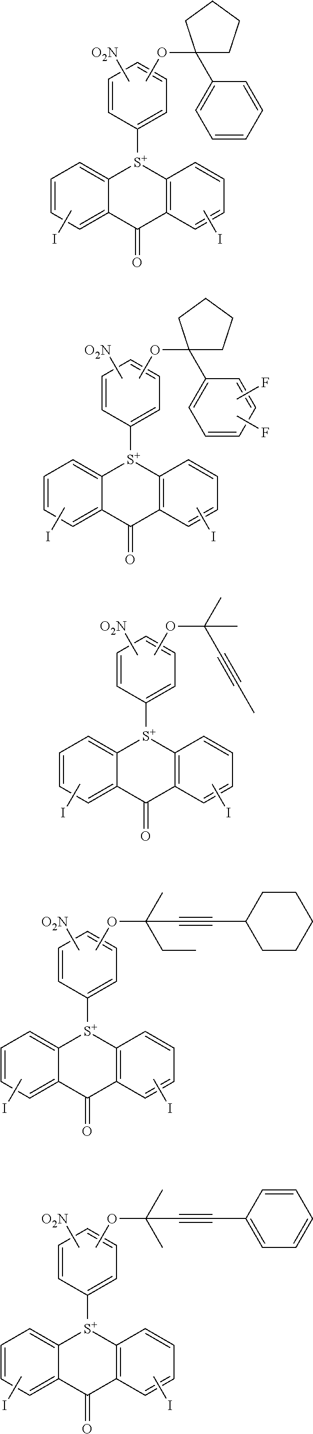

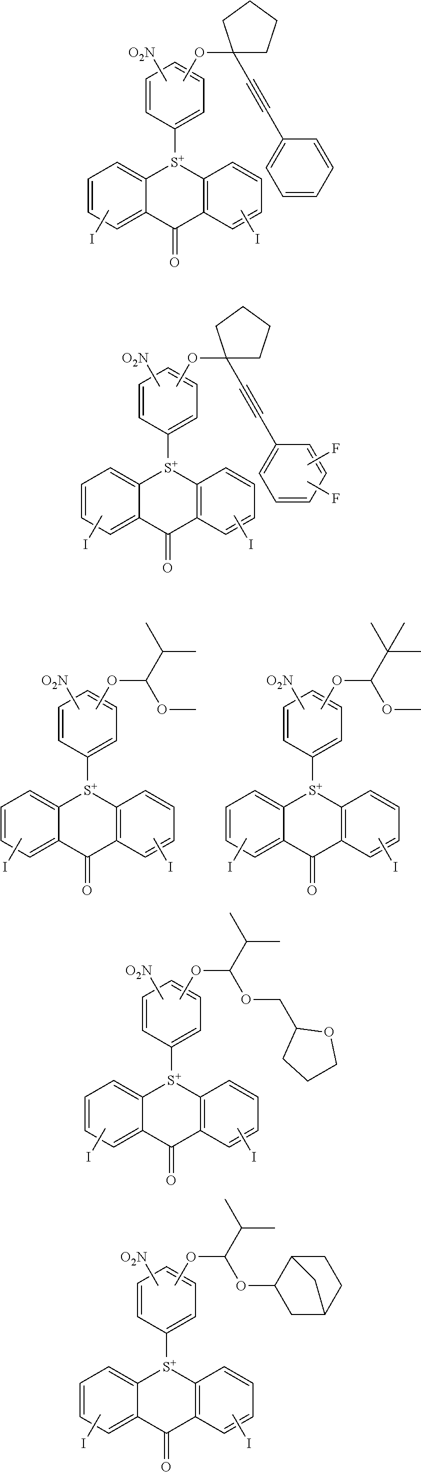

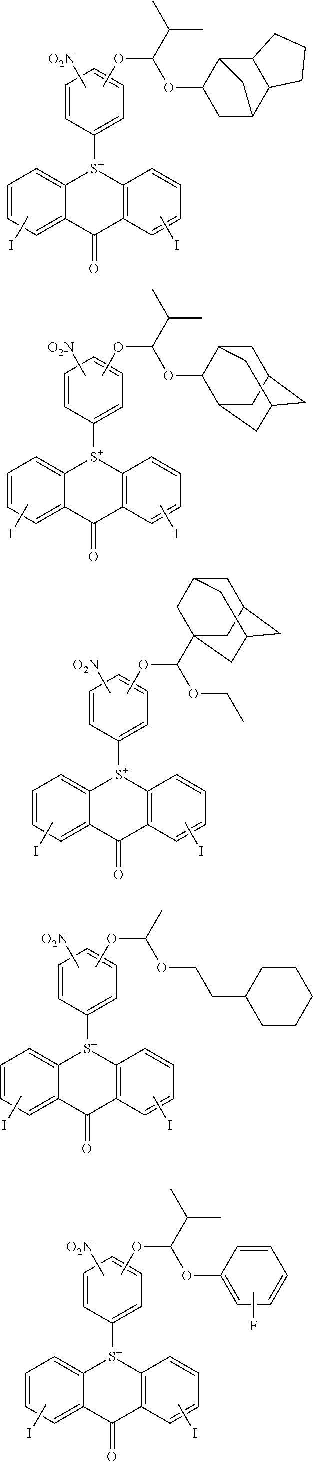

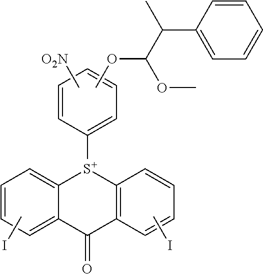

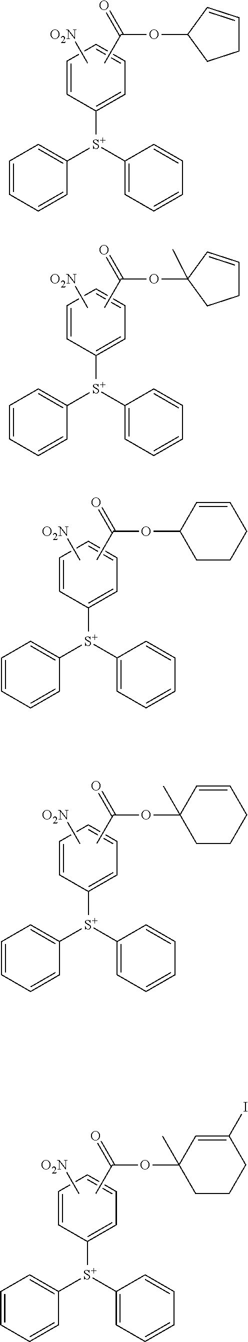

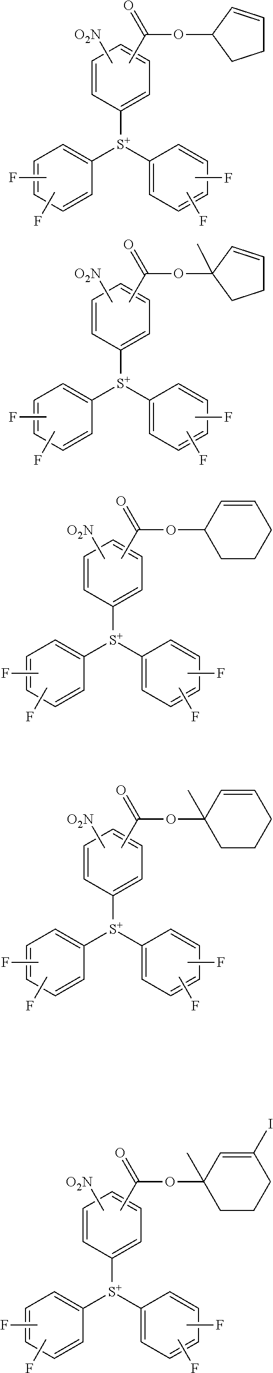

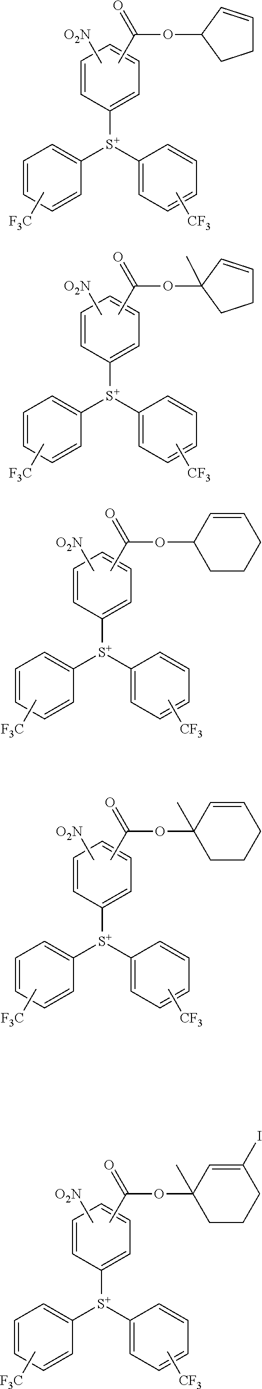

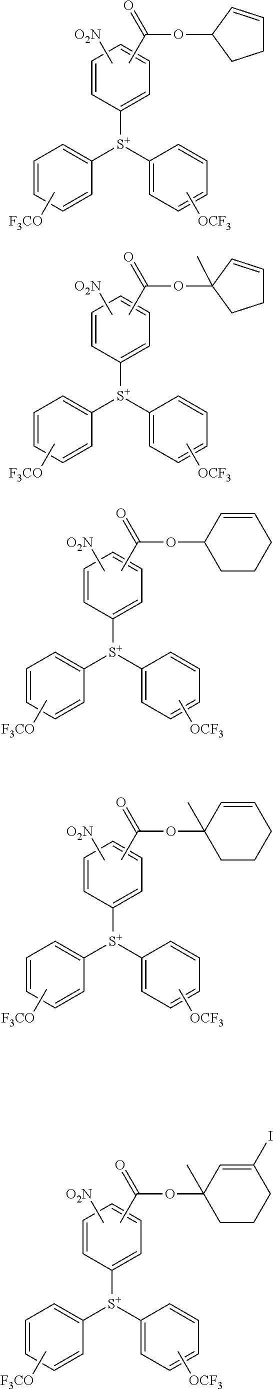

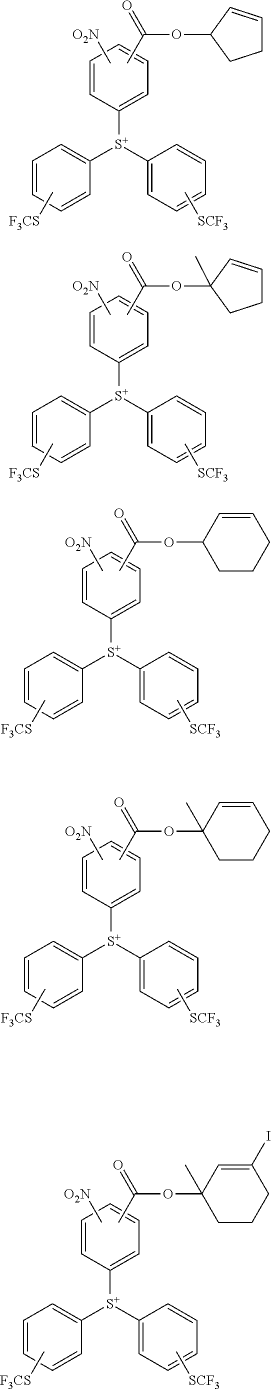

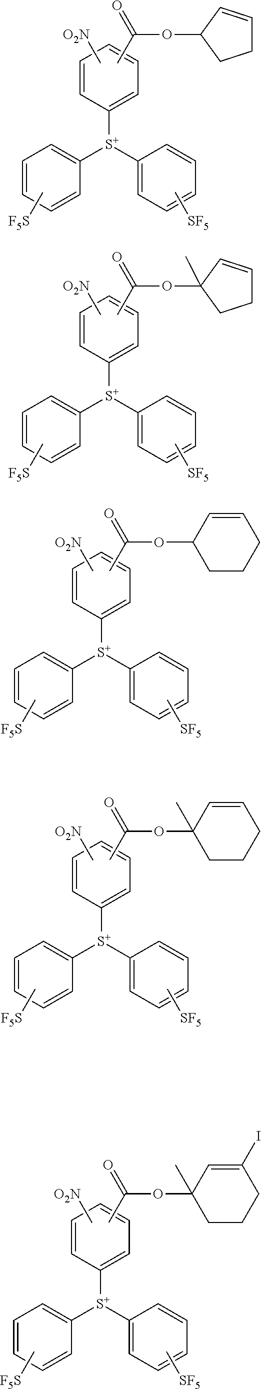

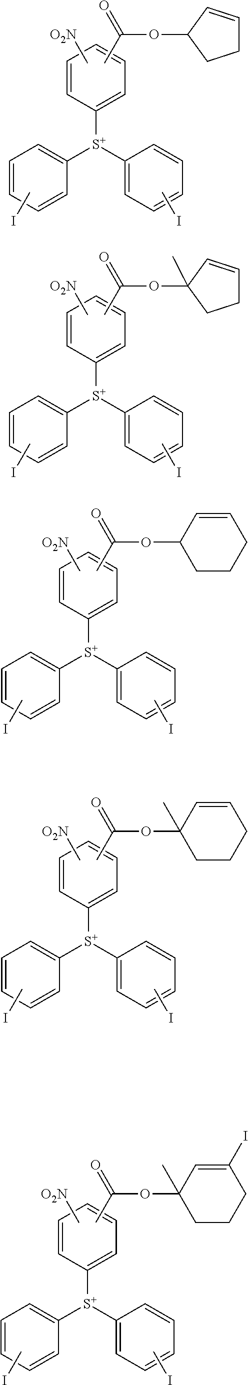

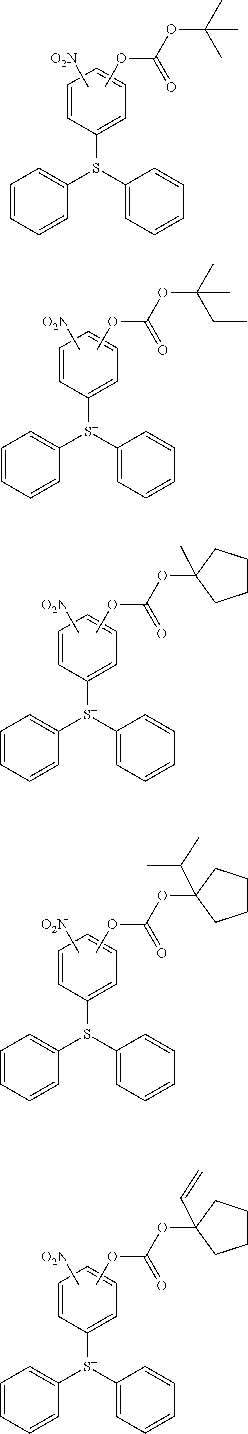

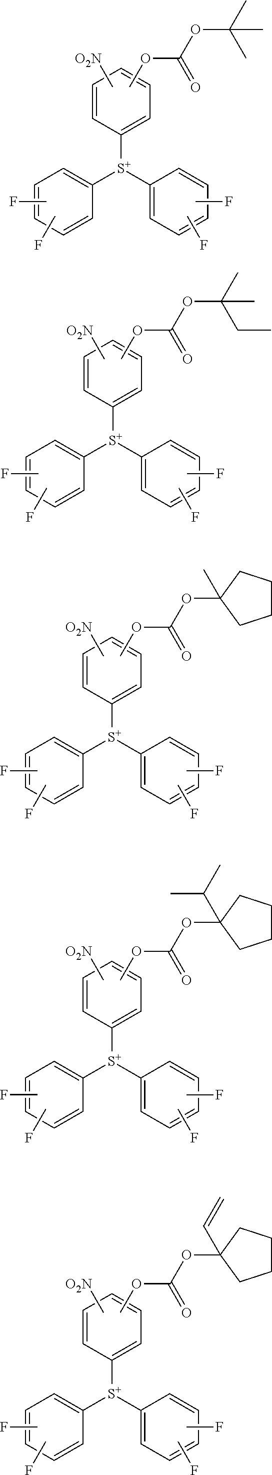

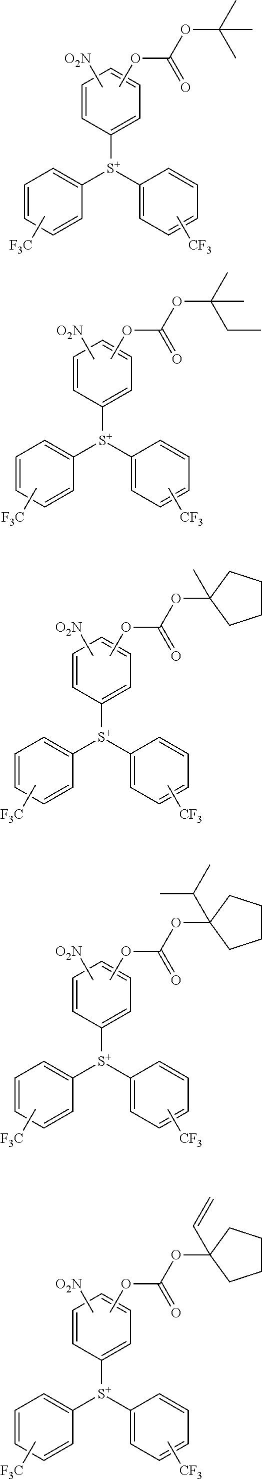

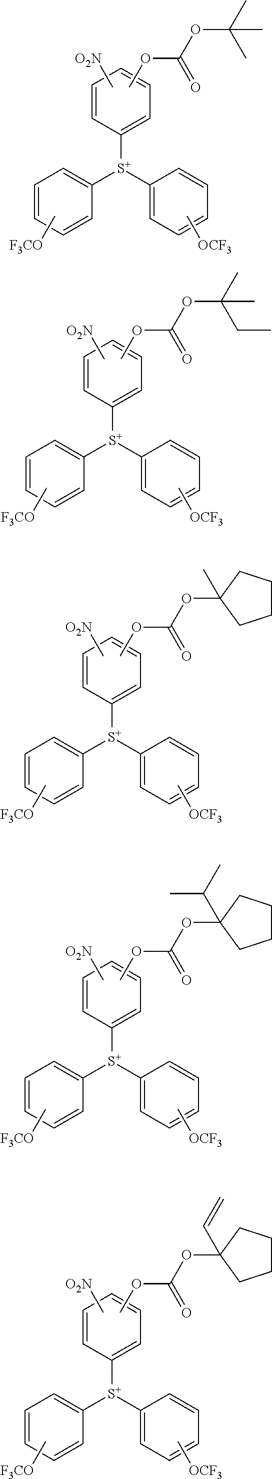

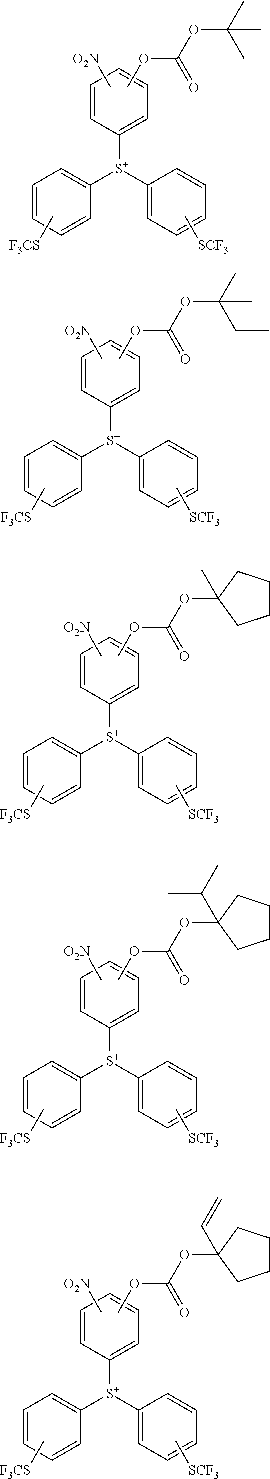

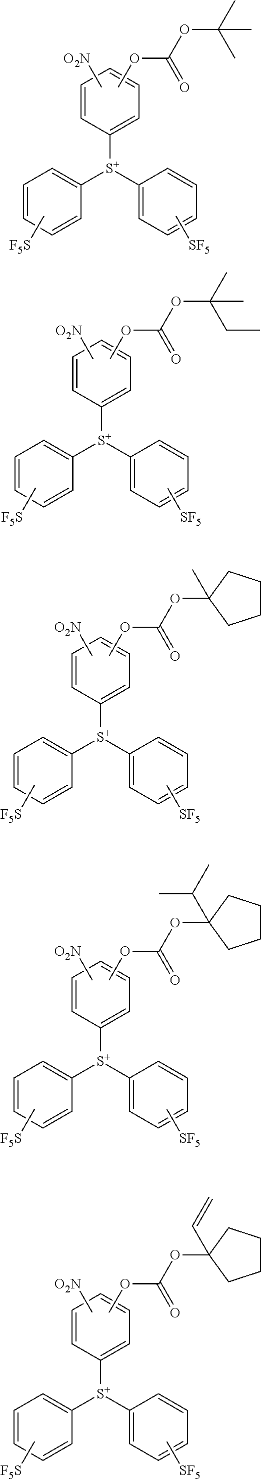

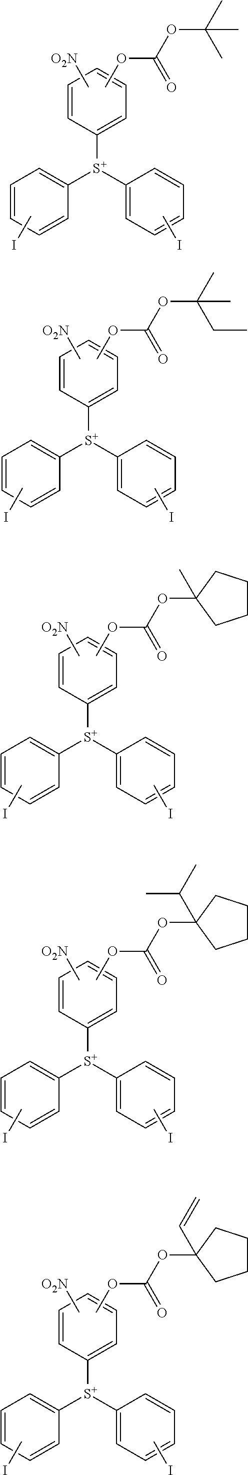

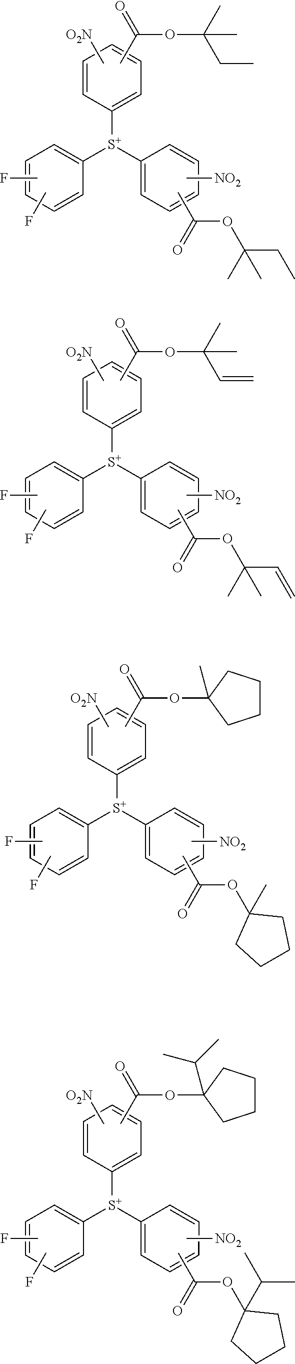

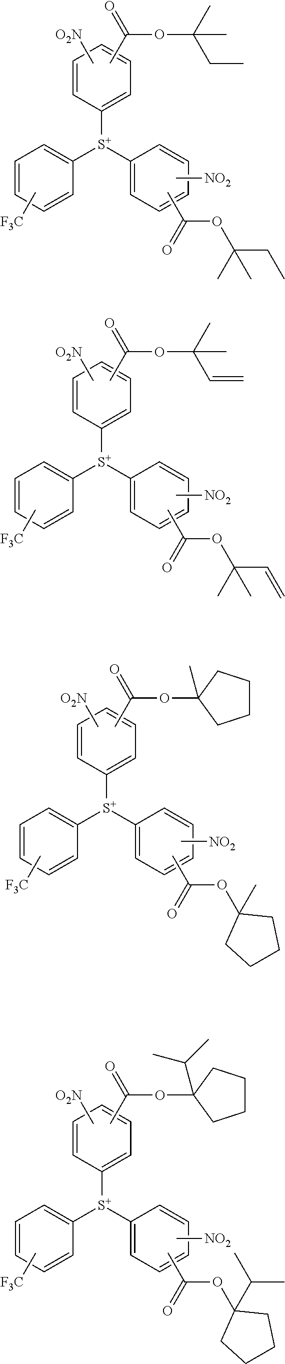

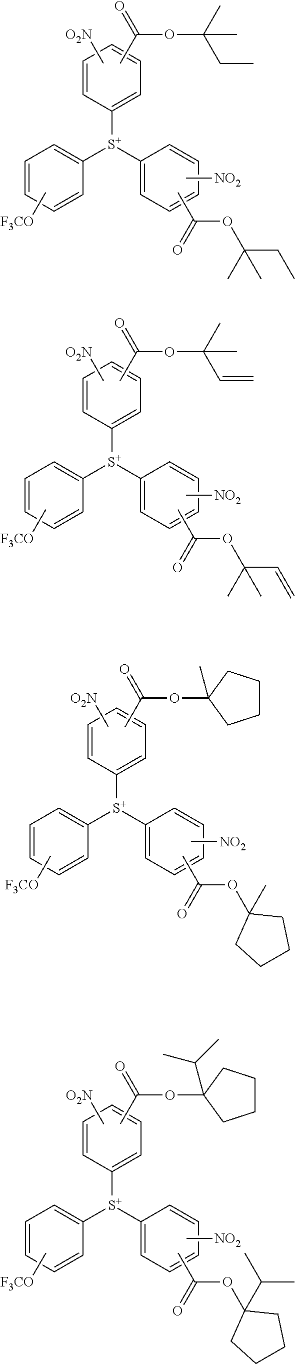

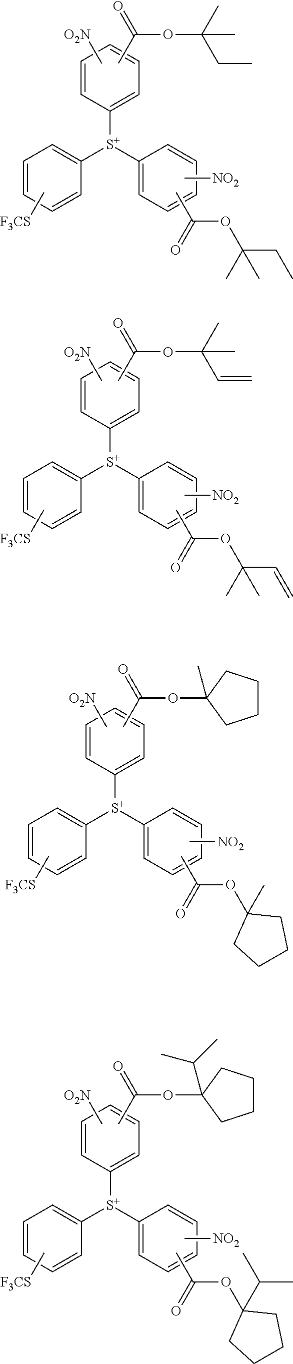

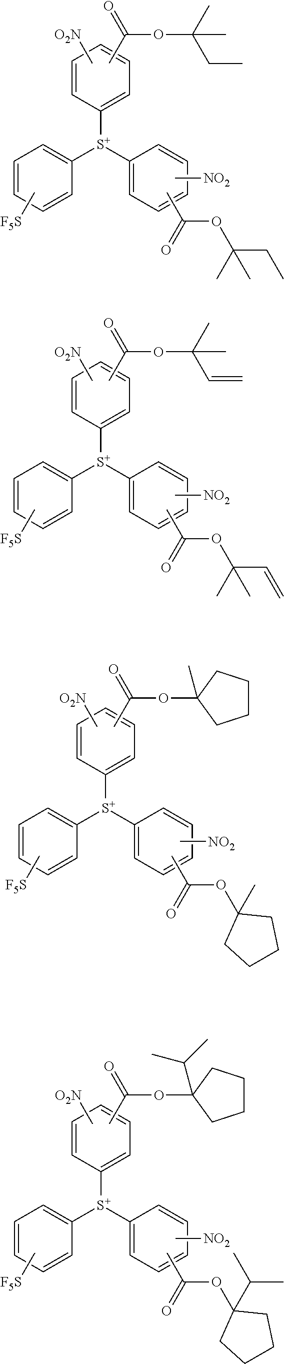

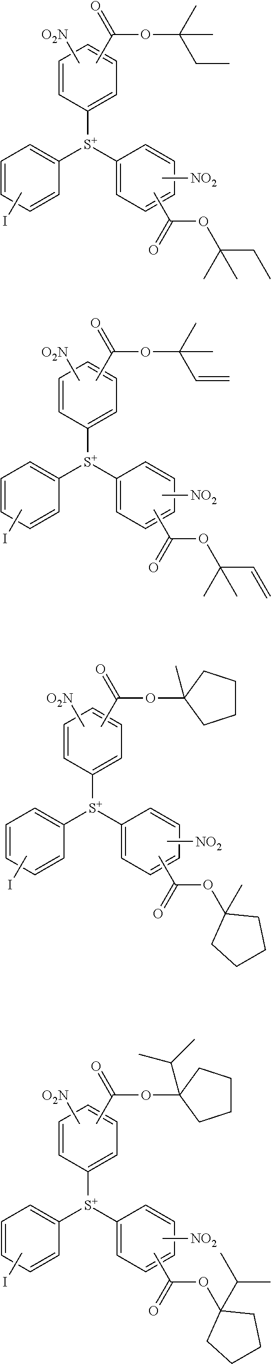

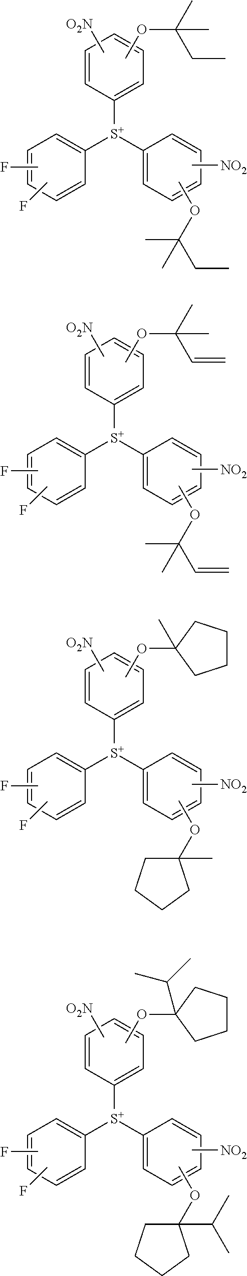

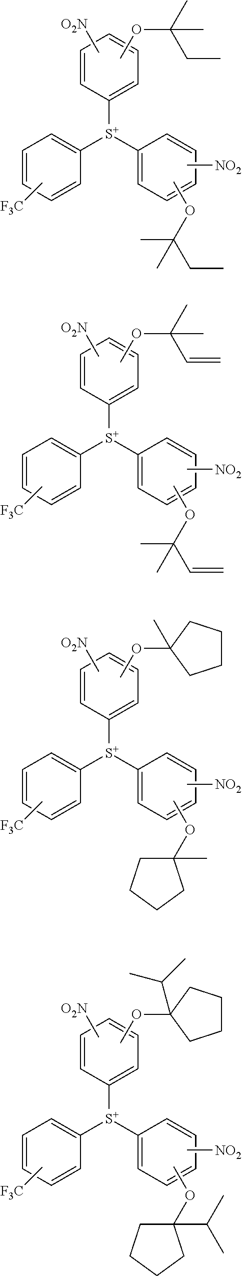

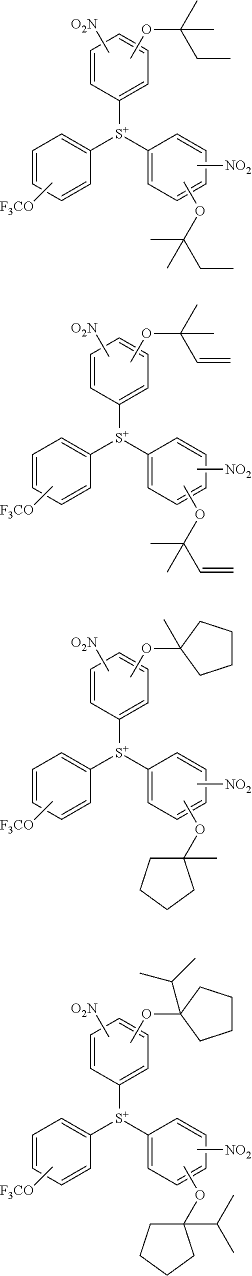

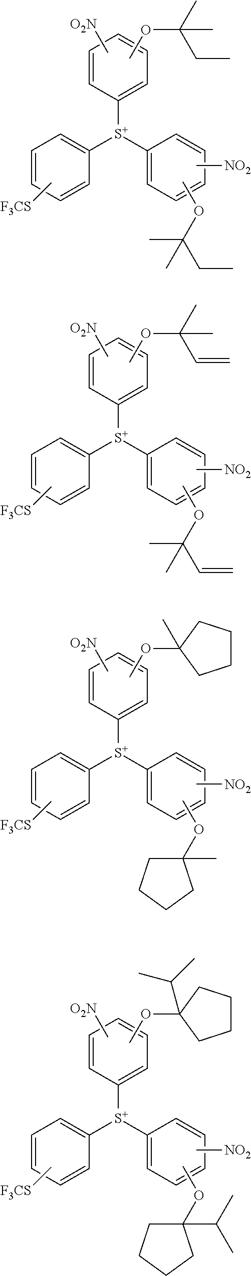

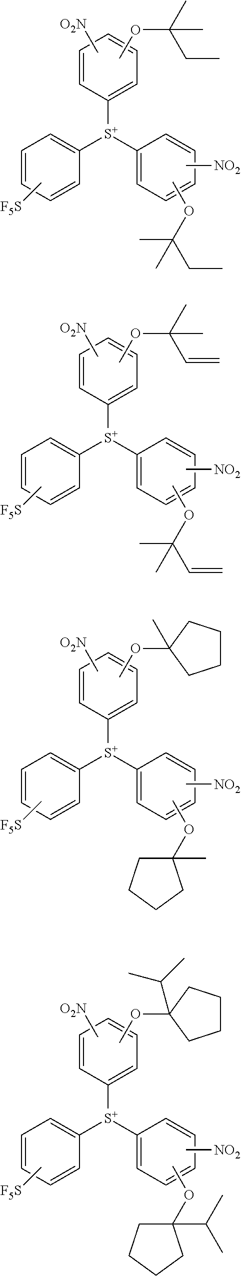

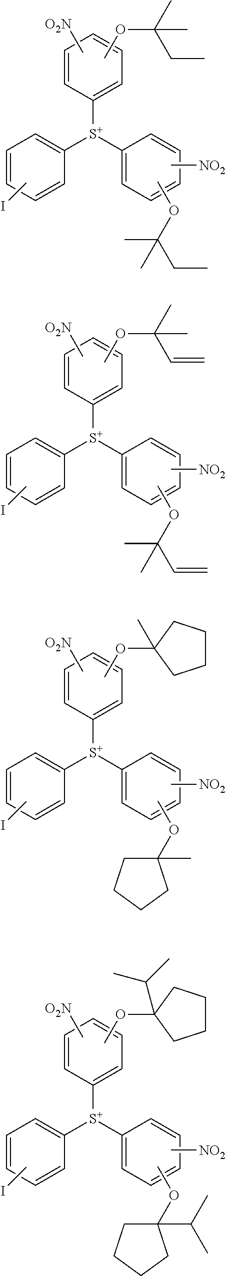

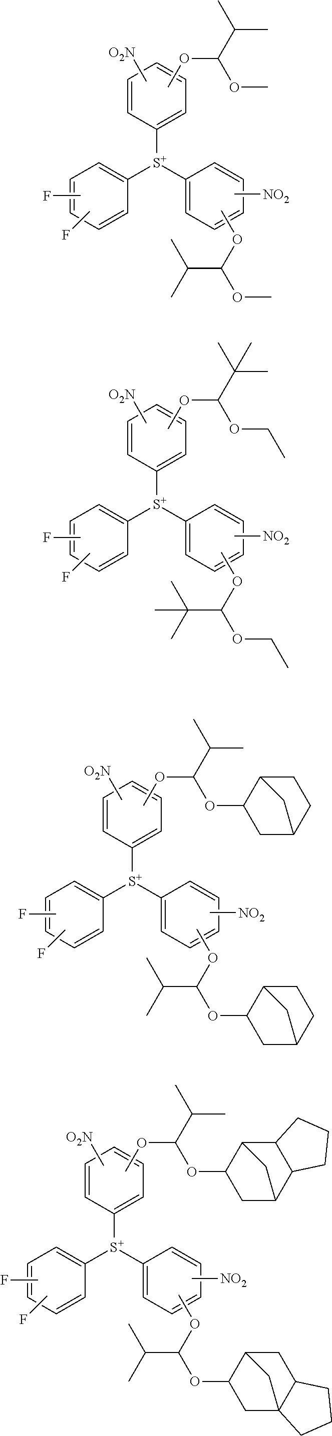

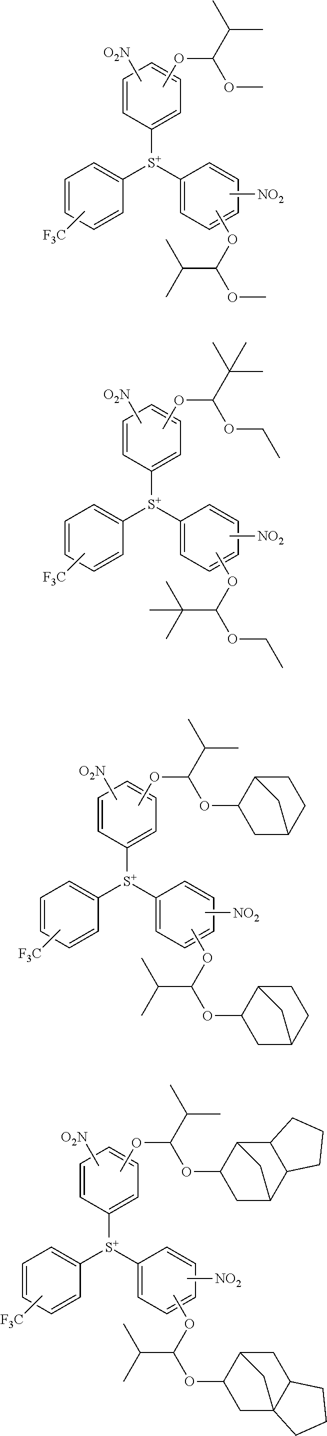

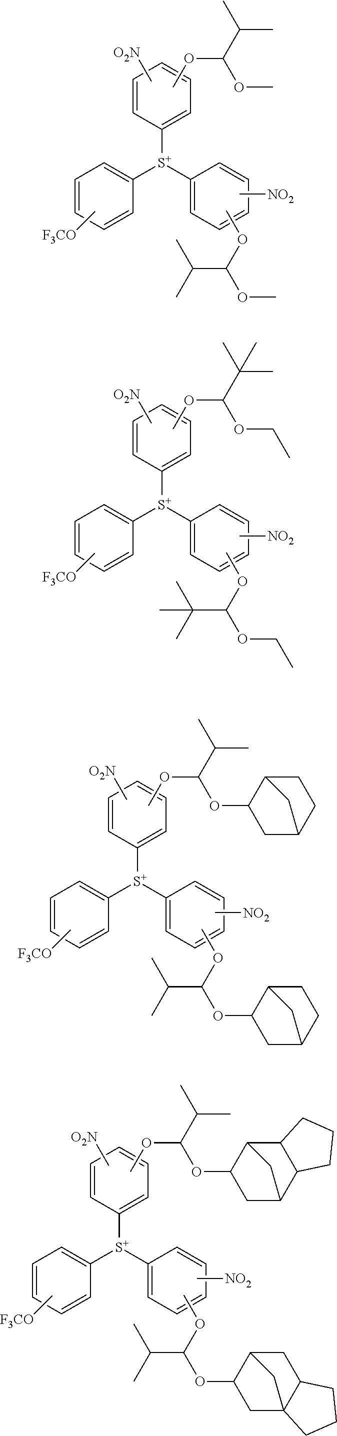

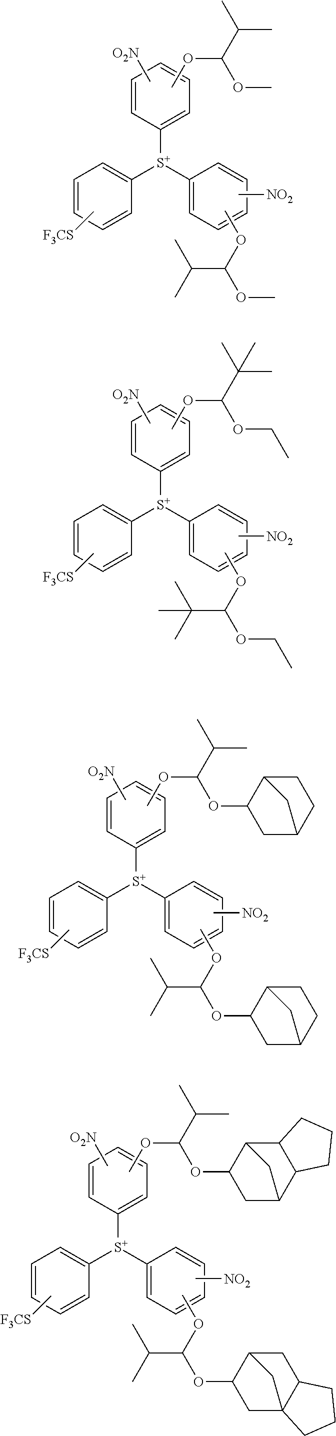

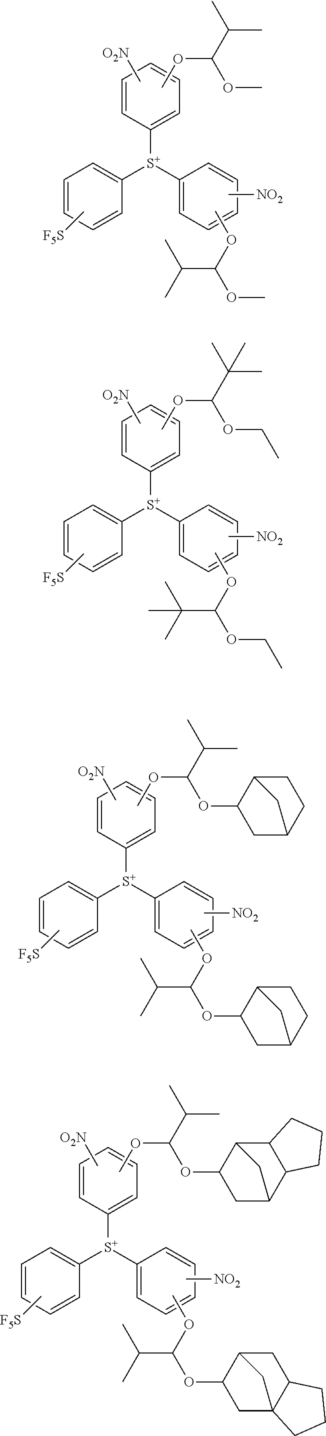

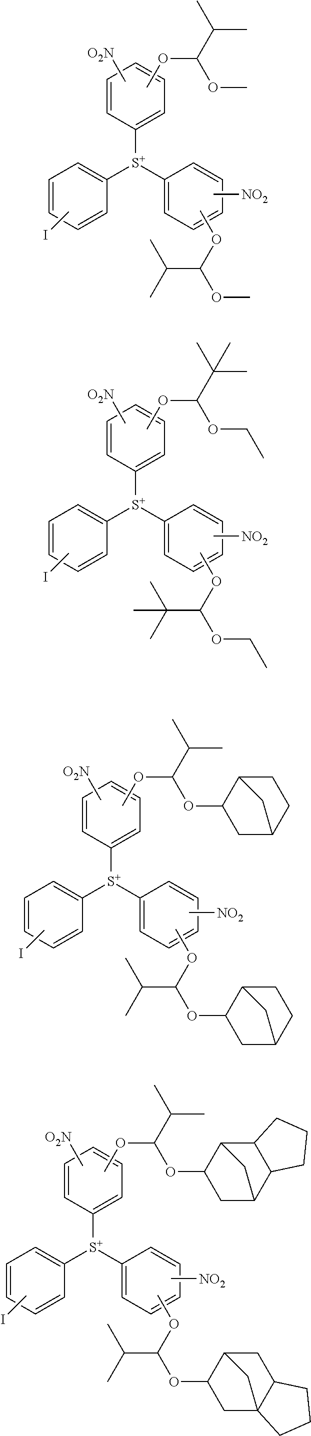

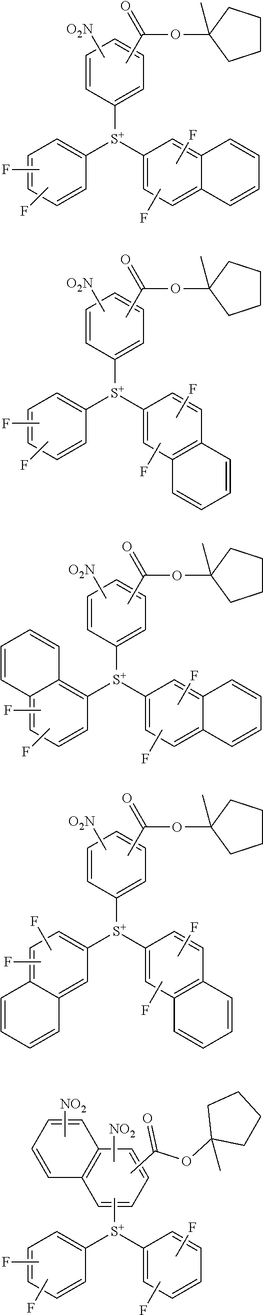

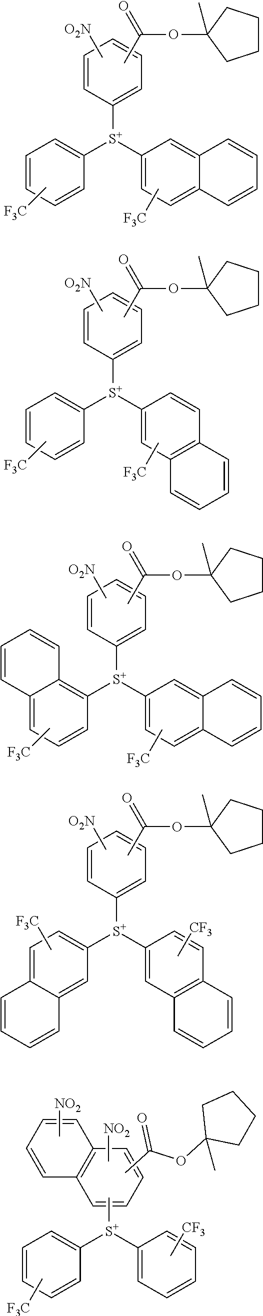

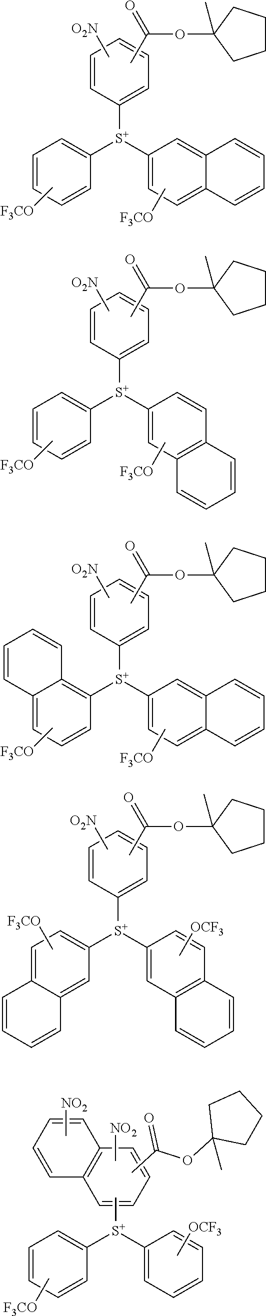

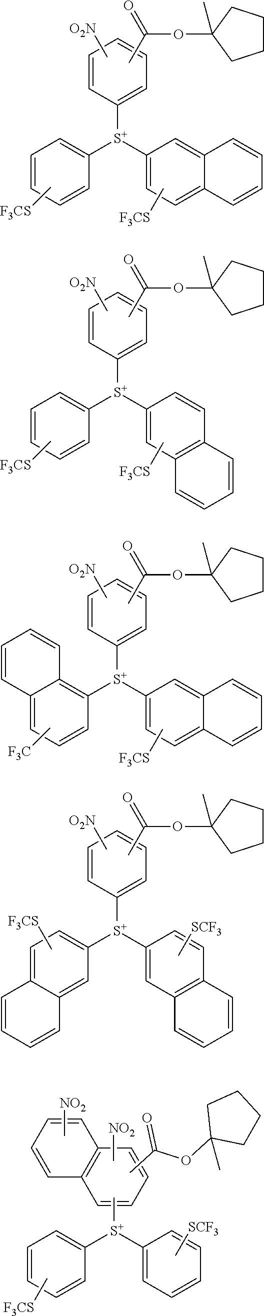

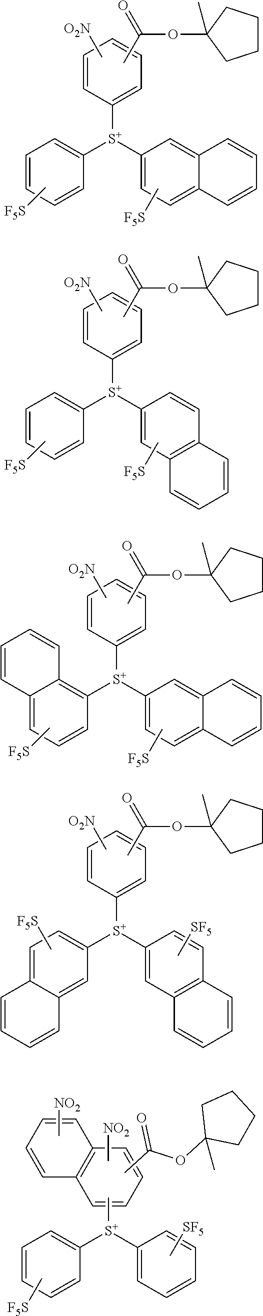

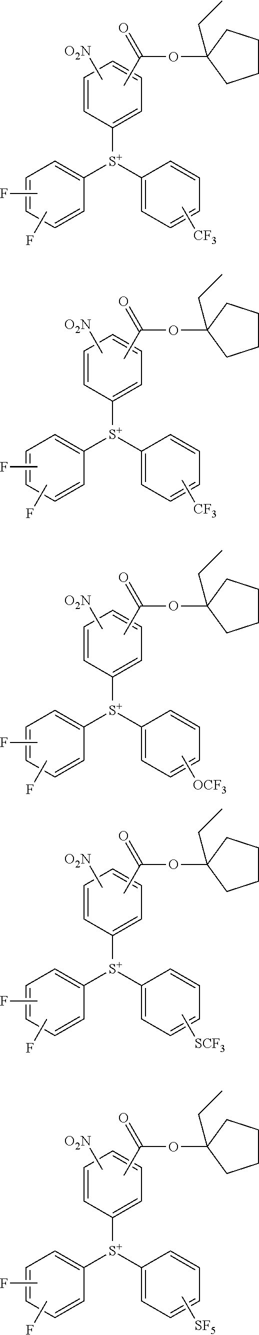

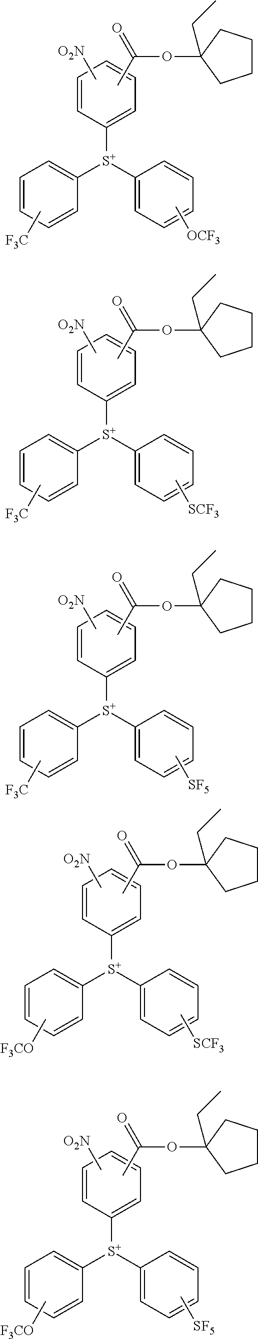

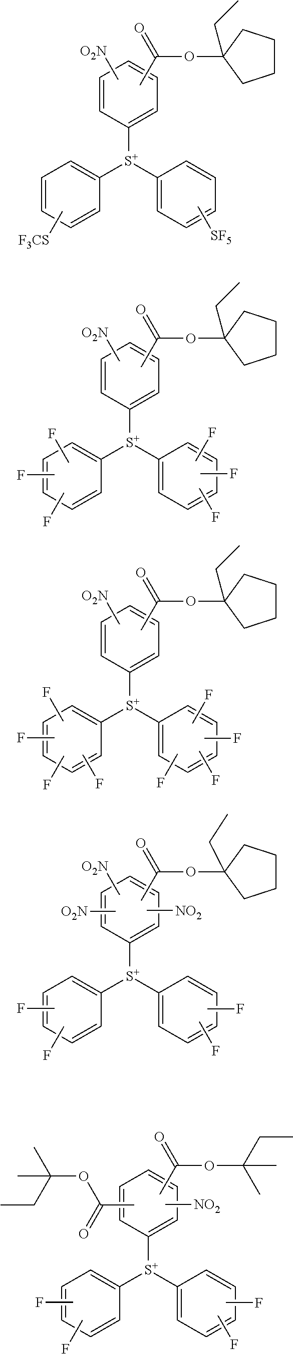

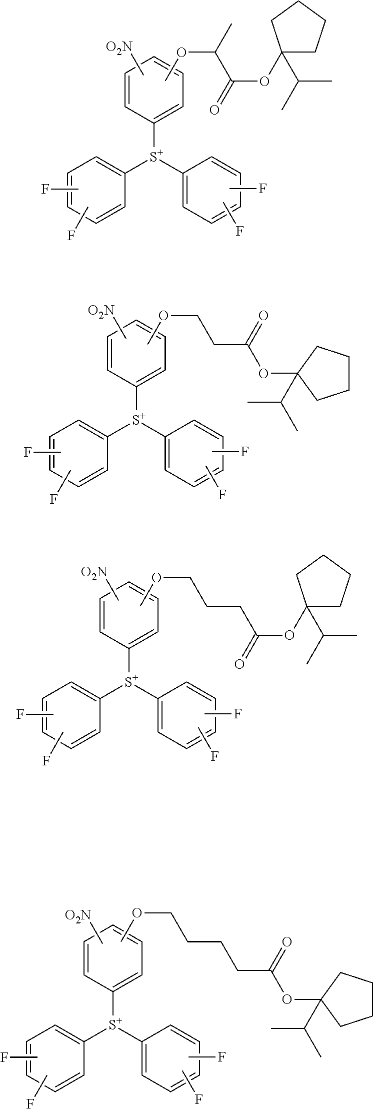

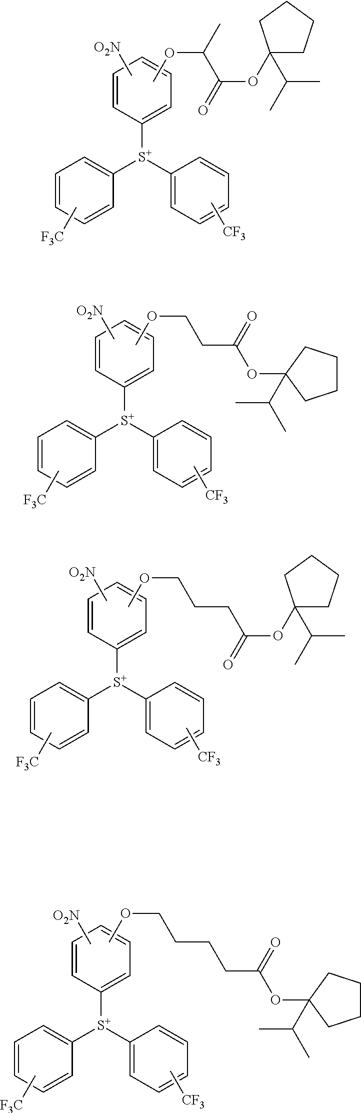

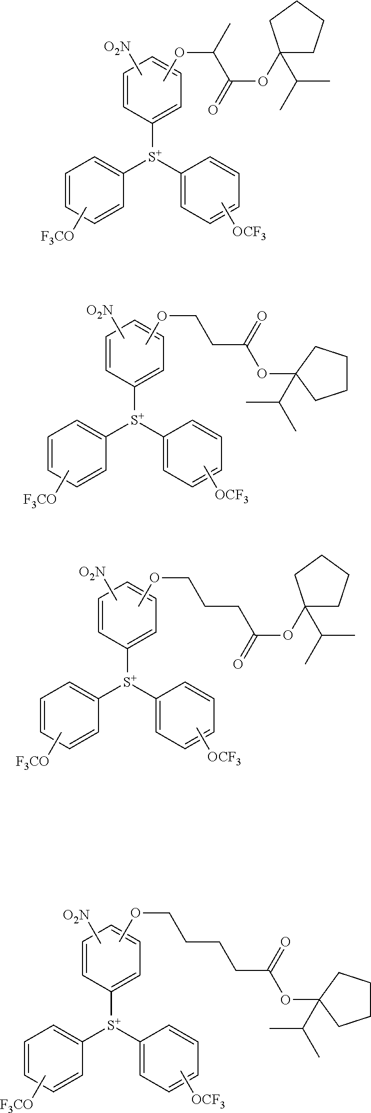

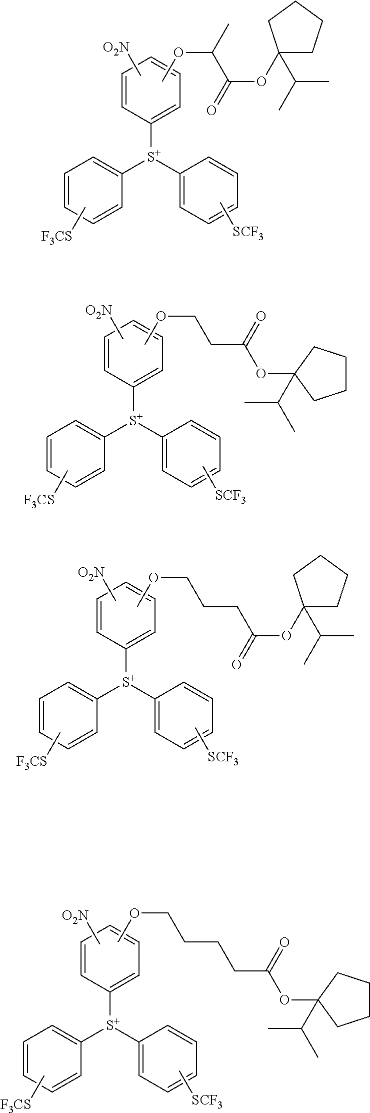

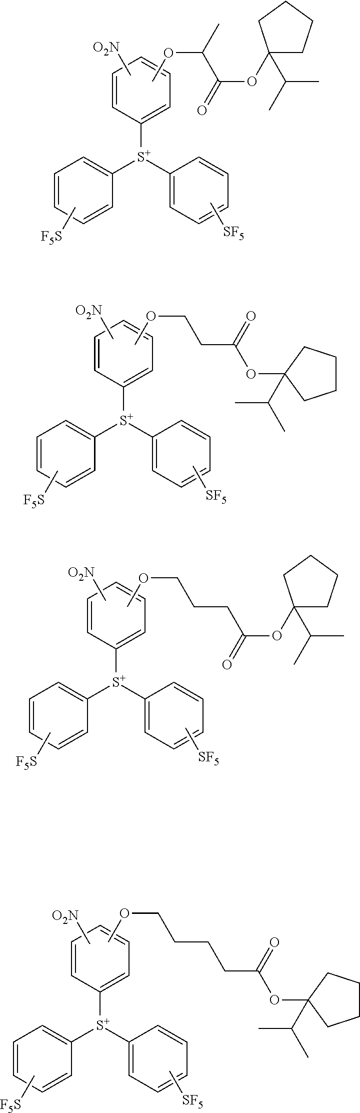

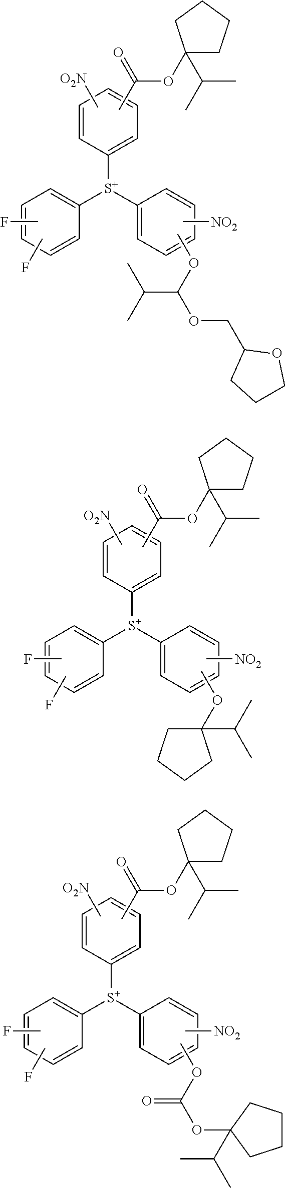

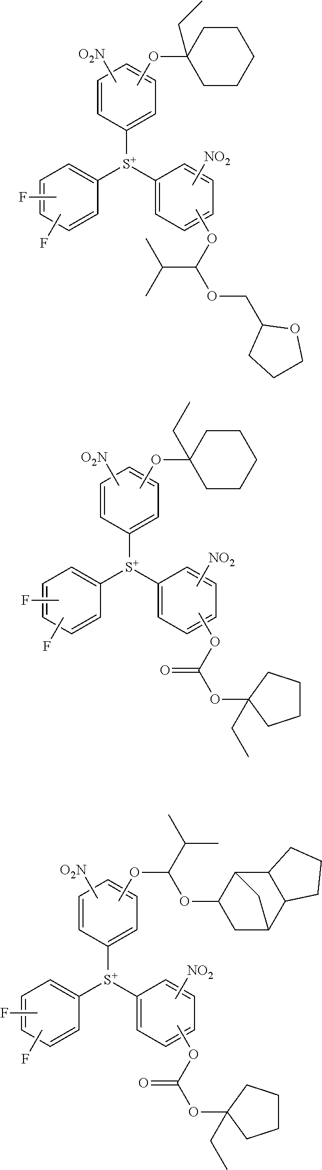

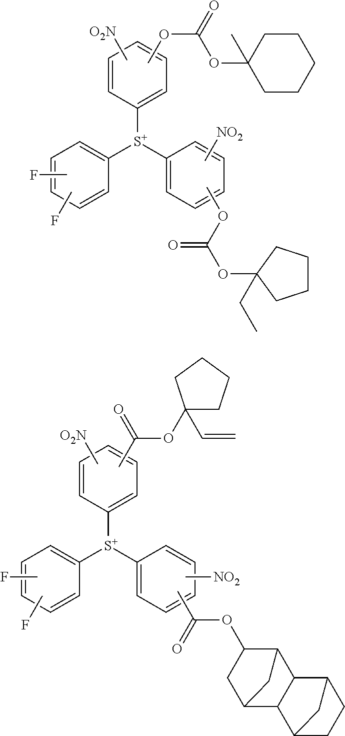

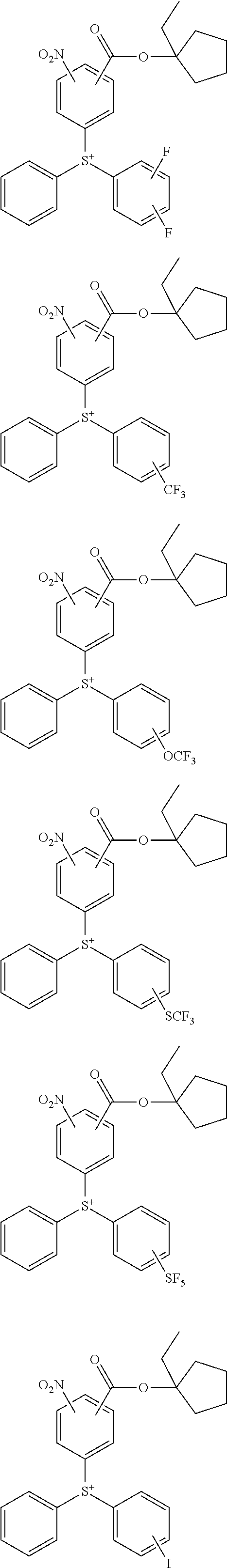

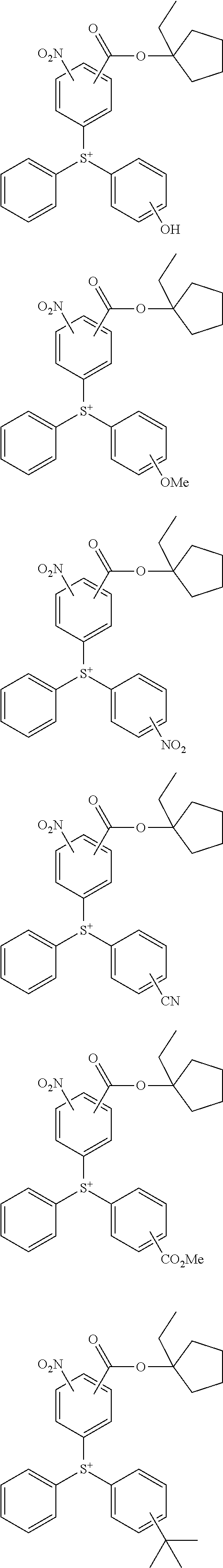

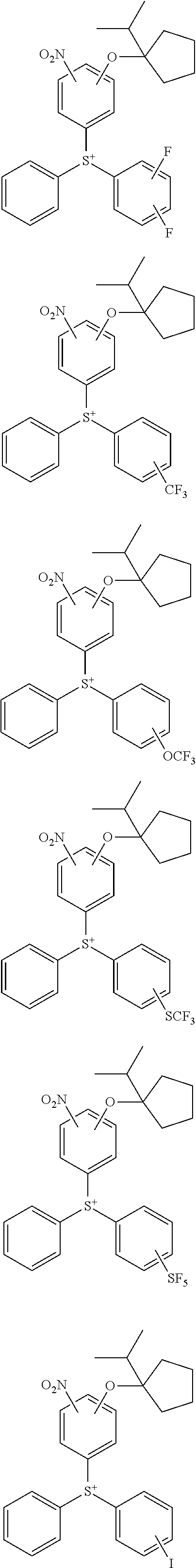

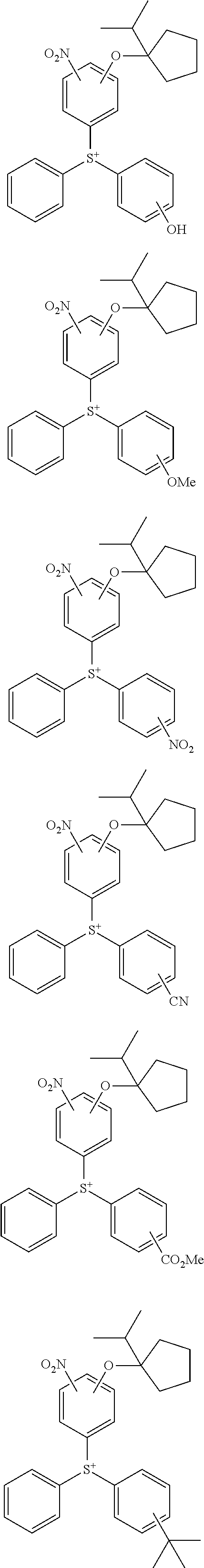

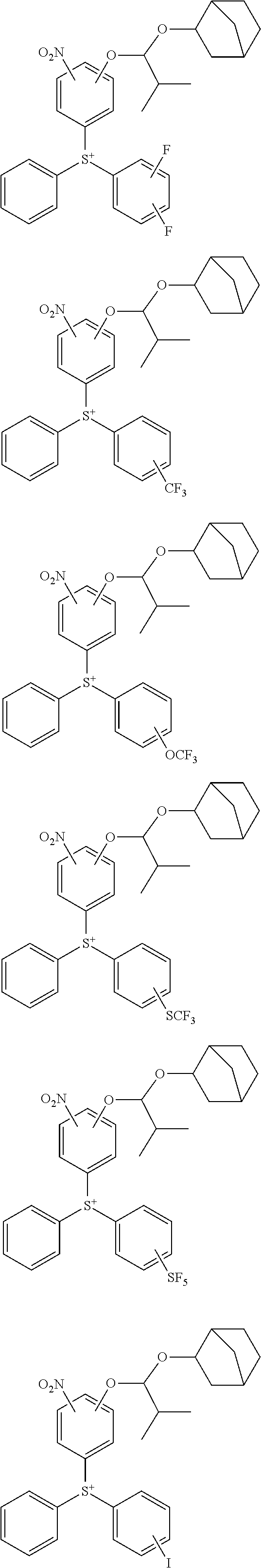

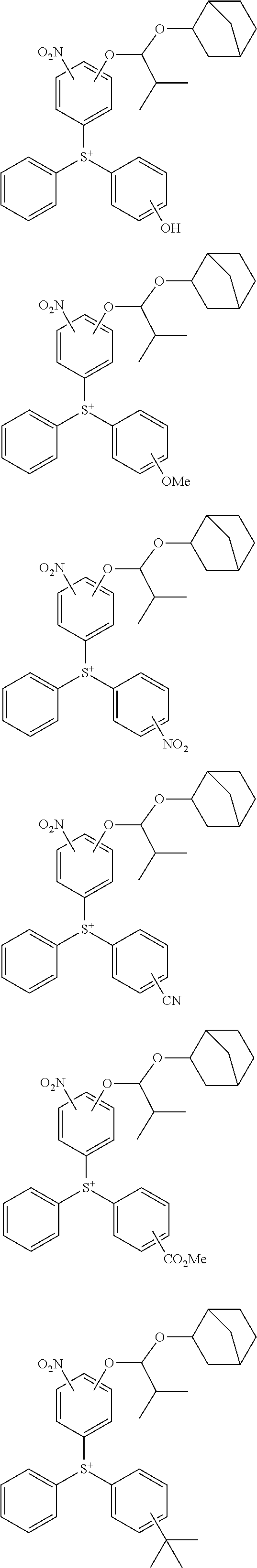

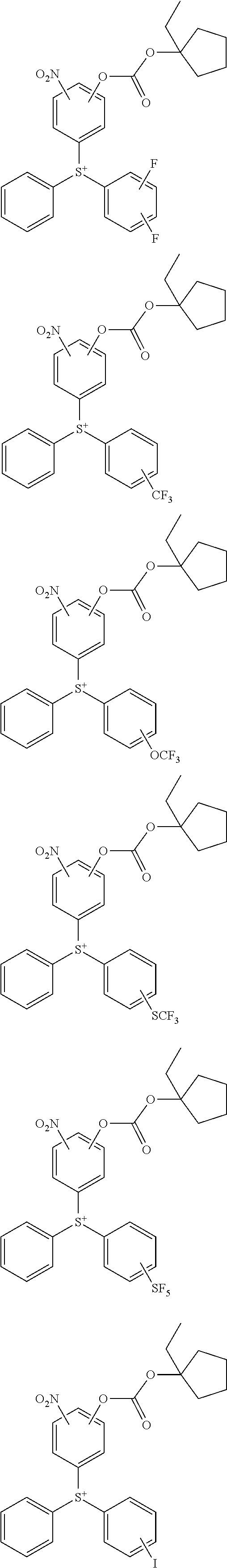

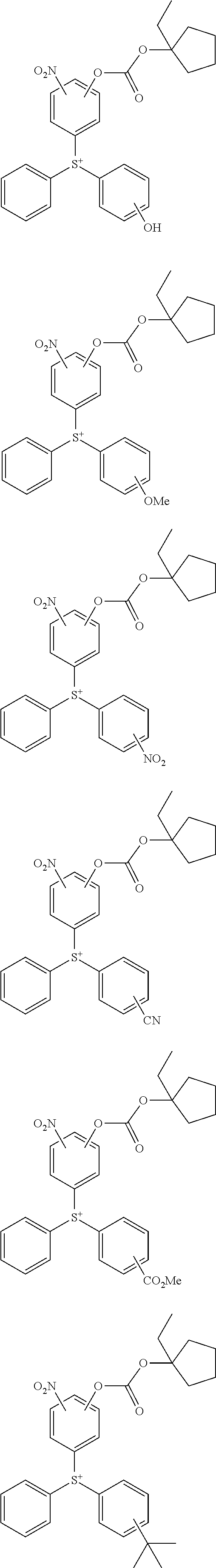

In the formulas (1) and (1A), Z⁻ is an aromatic carboxylic acid anion which may have a substituent. The aromatic carboxylic acid anion preferably has the following formula (Z):

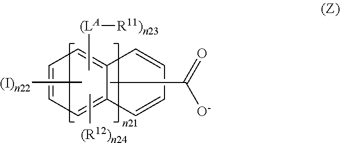

In the formula (Z), n21 is 0 or 1. When n21 is 0, it is a benzene ring, and when n21 is 1, it is a naphthalene ring, but a benzene ring in which n21 is 0 is preferable from a viewpoint of solvent solubility. n22 is 0, 1, 2, 3, or 4, more preferably 0, 1, 2, or 3 from a viewpoint of raw material procurement, and still more preferably 0, 1, or 2 from a viewpoint of solvent solubility. n23 is 0, 1, or 2. n24 is 0, 1, 2, 3, or 4. Provided that 2≤n22+n23+n24≤5 when n21 is 0, and 2≤n22+n23+n24≤7 when n21 is 1. n24 is preferably 0, 1, 2, or 3, and more preferably 0, 1, or 2 from a viewpoint of raw material procurement.

In the formula (Z), L^{A} is a single bond, —O—, —S—, an ester bond, or a carbonate bond, When R¹¹ described below is a hydrogen atom, L^{A} is preferably —O—. When R¹¹ described below is an acid-unstable group, L^{A} is preferably —O—, an ester bond, or a carbonate bond, and more preferably —O— or an ester bond.

In the formula (Z), R¹¹ is a hydrogen atom or a C₁-C₂₀ hydrocarbyl group which may contain a heteroatom when L^{A} is a single bond. The C₁-C₂₀ hydrocarbyl group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a tert-pentyl group, a n-pentyl group, a n-hexyl group, a n-octyl group, a 2-ethylhexyl group, a n-nonyl group, a n-decyl group, a n-undecyl group, or a n-dodecyl group; a C₃-C₂₀ cyclic saturated hydrocarbyl group such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a norbornylmethyl group, an adamantyl group, an adamantylmethyl group, a tricyclo [5.2.1.0^{2,6}] decyl group, or a tetracyclo [6.2.1.1^{3,6}.0^{2,7}] dodecyl group; a C₂-C₂₀ alkenyl group such as a vinyl group, a 1-propenyl group, a 2-propenyl group, a butenyl group, a pentenyl group, or a hexenyl group; a C₂-C₂₀ alkynyl group such as an ethynyl group, a propynyl group, a butynyl group, a pentynyl group, or a hexynyl group; a C₃-C₂₀ cyclic unsaturated aliphatic hydrocarbyl group such as a cyclopentenyl group or a cyclohexenyl group; a C₆-C₂₀ aryl group such as a phenyl group, a naphthyl group, or an indanyl group; a C₇-C₂₀ aralkyl group such as a benzyl group, a 1-phenylethyl group, or a 2-phenylethyl group, and a group obtained by combining these groups. The hydrocarbyl group may contain a heteroatom, and specifically may contain a halogen atom, an oxygen atom, a sulfur atom, or the like.

R¹¹ is a hydrogen atom, a C₁-C₂₀ hydrocarbyl group (except for an acid-unstable group) which may contain a heteroatom, or an acid-unstable group when L^{A} is —O—, —S—, an ester bond, or a carbonate bond. Examples of the hydrocarbyl group include those other than an acid-unstable group among those described above.

The acid-unstable group of R¹¹ preferably has the following formula (AL-3) or (AL-4):

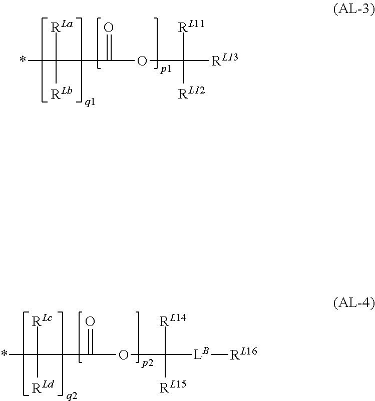

wherein * represents a bond with L^{A}.

In the formulas (AL-3) and (AL-4), p1 and p2 are each independently 0 or 1. q1 and q2 are each independently 0, 1, 2, 3, or 4. When p1 is 1, q1 is preferably 1 or 2, and when p1 is 0, q1 is 0. When p2 is 1, q2 is preferably 1 or 2, and when p2 is 0, q2 is 0.

In the formula (AL-3), R^{L11} and R^{L12} are each independently a C₁-C₁₂ hydrocarbyl group. R^{L13} is a hydrogen atom or a C₁-C₁₂ hydrocarbyl group. Some of the (—CH₂-)s of the hydrocarbyl group of R^{L11}, R^{L12}, and R^{L13} may be replaced with —O— or —S—, when the hydrocarbyl group contains an aromatic ring, some or all of the hydrogen atoms of the aromatic ring may be replaced with a halogen atom, a cyano group, a nitro group, a C₁-C₄ alkyl group which may contain a halogen atom, or a C₁-C₄ alkoxy group which may contain a halogen atom.

The C₁-C₁₂ hydrocarbyl groups of R^{L11}, R^{L12}, and R^{L13} may be saturated or unsaturated, and may be linear, branched, or cyclic. Specific examples thereof include: a C₁-C₁₂ alkyl group such as a methyl group, an ethyl group, a n-propyl group, an isopropyl group, a n-butyl group, an isobutyl group, a sec-butyl group, a tert-butyl group, a tert-pentyl group, a n-pentyl group, a n-hexyl group, a n-octyl group, a 2-ethylhexyl group, a n-nonyl group, a n-decyl group, a n-undecyl group, or a n-dodecyl group; a C₃-C₁₂ cyclic saturated hydrocarbyl group such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a norbornylmethyl group, an adamantyl group, an adamantylmethyl group, a tricyclo [5.2.1.0^{2,6}] decyl group, or a tetracyclo [6.2.1.1^{3,6}.0^{2,7}] dodecyl group; a C₂-C₁₂ alkenyl group such as a vinyl group, a 1-propenyl group, a 2-propenyl group, a butenyl group, a pentenyl group, or a hexenyl group; a C₂-C₁₂ alkynyl group such as an ethynyl group, a propynyl group, a butynyl group, a pentynyl group, or a hexynyl group; a C₃-C₁₂ cyclic unsaturated aliphatic hydrocarbyl group such as a cyclopentenyl group or a cyclohexenyl group; a C₆-C₁₂ aryl group such as a phenyl group, a naphthyl group, or an indanyl group; a C₇-C₁₂ aralkyl group such as a benzyl group, a 1-phenylethyl group, or a 2-phenylethyl group, and a group obtained by combining these groups.

R^{L11} and R^{L12} may be bonded to each other to form a ring together with a carbon atom to which they are bonded, and some of the (—CH₂-)s of the ring may be replaced with —O— or —S—. Specific examples of the ring formed at this time include a cyclopropane ring, a cyclobutane ring, a cyclopentane ring, a cyclohexane ring, a cycloheptane ring, a cyclooctane ring, a norbornane ring, an adamantane ring, a tricyclo [5.2.1.0^{2,6}] decane ring, and a tetracyclo [6.2.1.1^{3,6}.0^{2,7}] dodecane ring. Some of the (—CH₂-)s of the ring may be replaced with —O— or —S—. Provided that when R^{L13} is a hydrogen atom, R^{L11} and R^{L12} are bonded to each other to form an alicyclic ring containing a multiple bond together with a carbon atom to which they are bonded. Some of the hydrogen atoms of the alicyclic ring may be replaced with halogen atoms.

In the formula (AL-4), R^{L14} and R^{L15} are each independently a hydrogen atom or a C₁-C₁₀ hydrocarbyl group. The C₁-C₁₀ hydrocarbyl groups of R^{L14} and R^{L15} may be saturated or unsaturated, and may be linear, branched, or cyclic. Specific examples thereof include those similar to those having 1 to 10 carbon atoms among those exemplified as the hydrocarbyl groups of R^{L11}, R^{L12}, and R^{L13}.

In the formula (AL-4), R^{L16} is a C₁-C₂₀ hydrocarbyl group, and some of the (—CH₂-)s of the hydrocarbyl group may be replaced with —O— or —S—. The hydrocarbyl group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, a norbornylmethyl group, an adamantyl group, an adamantylmethyl group, a tricyclo [5.2.1.0^{2,6}] decyl group, or a tetracyclo [6.2.1.1^{3,6}.0^{2,7}] dodecyl group; a C₂-C₂₀ alkenyl group such as a vinyl group, a propenyl group, a butenyl group, a pentenyl group, or a hexenyl group; a C₂-C₂₀ alkynyl group such as an ethynyl group, a propynyl group, a butynyl group, a pentynyl group, or a hexynyl group; a C₃-C₂₀ cyclic unsaturated aliphatic hydrocarbyl group such as a cyclopentenyl group, a cyclohexenyl group, or a norbornenyl group; a C₆-C₂₀ aryl group such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, or a tert-butylnaphthyl group; a C₇-C₂₀ aralkyl group such as a benzyl group or a phenethyl group; and a group obtained by combining these groups. R^{L15} and R^{L16} may be bonded to each other to form a C₃-C₂₀ heterocyclic group together with a carbon atom to which they are bonded and L^{B}, and some of the (—CH₂-)s of the heterocyclic group may be replaced with —O— or —S—.

In the formula (AL-4), L^{B} is —O— or —S—.

In the formulas (AL-3) and (AL-4), R^{La} to R^{Ld} are each independently a hydrogen atom or a C₁-C₂₀ hydrocarbyl group which may contain a heteroatom. The C₁-C₂₀ hydrocarbyl group may be saturated or unsaturated, and may be linear, branched, or cyclic. Specific examples thereof include, but are not limited to, those similar to those exemplified as the hydrocarbyl group of R¹¹.

Specific examples of the acid-unstable group having the formula (AL-3) include, but are not limited to, those similar to those exemplified as the acid-unstable group having the formula (AL-1).

Specific examples of the acid-unstable group having the formula (AL-4) include, but are not limited to, those similar to those exemplified as the acid-unstable group having the formula (AL-2).

In the formula (Z), R¹² is a halogen atom other than an iodine atom, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Examples of the halogen atom other than an iodine atom include a fluorine atom, a chlorine atom, and a bromine atom, and the halogen atom is preferably a fluorine atom. The C₁-C₂₀ hydrocarbyl group may be saturated or unsaturated, and may be linear, branched, or cyclic. Specific examples thereof include those similar to those exemplified as the hydrocarbyl group of R¹¹. The hydrocarbyl group may contain a heteroatom, and specifically may contain a halogen atom, an oxygen atom, a sulfur atom, or the like.

When n24 is 2, 3, or 4, a plurality of R¹²s may be bonded to each other to form a ring structure together with carbon atoms on aromatic rings to which R¹²s are bonded, and some of the (—CH₂-)s of the ring may be replaced with —O— or —S—. Specific examples of the ring formed at this time include a cyclopropane ring, a cyclobutane ring, a cyclopentane ring, a cyclohexane ring, a cycloheptane ring, a cyclooctane ring, a norbornane ring, an adamantane ring, a tricyclo [5.2.1.0^{2,6}] decane ring, and a tetracyclo [6.2.1.1^{3,6}.0^{2,7}] dodecane ring. Some of the (—CH₂-)s of the ring may be replaced with —O— or —S—.

The aromatic carboxylic acid anion having the formula (Z) preferably has the following formula (Z1):

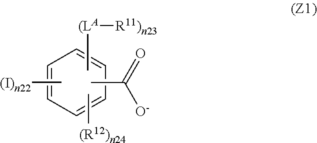

wherein n22 to n24, L^{A}, R¹¹, and R¹² are as defined above.

Specific examples of the aromatic carboxylic acid anion having the formula (Z) include, but are not limited to, those listed below.

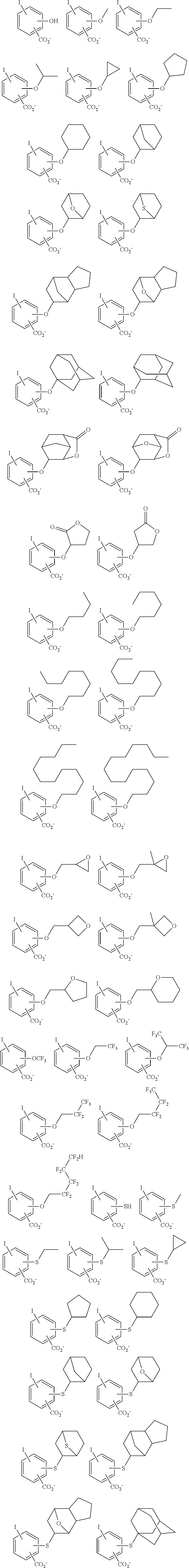

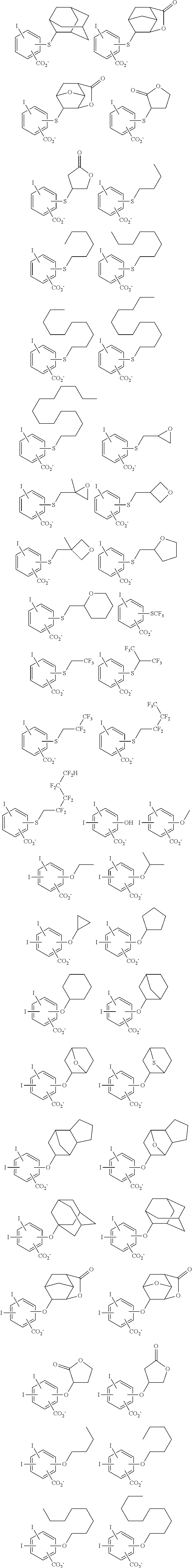

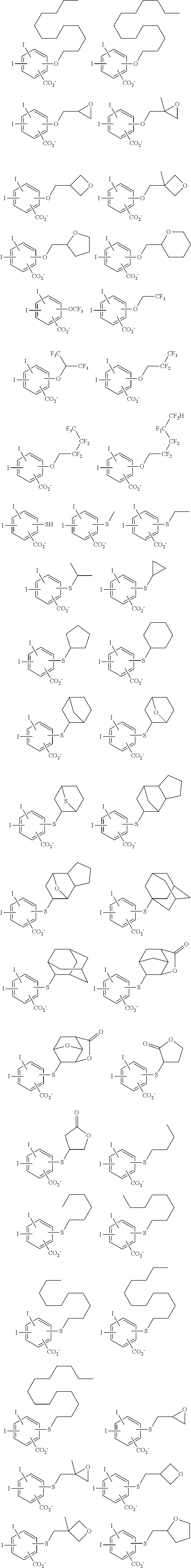

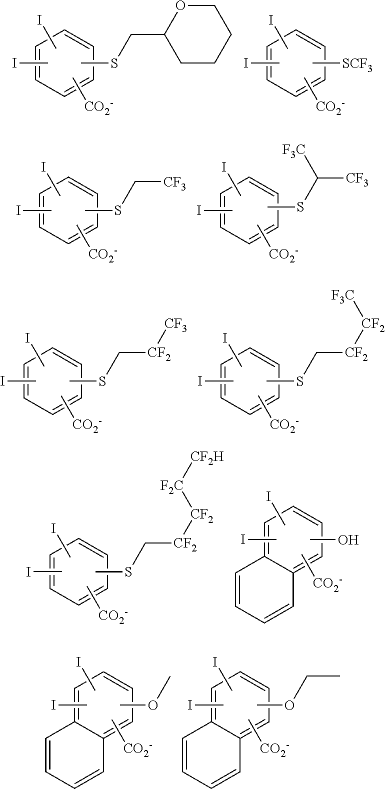

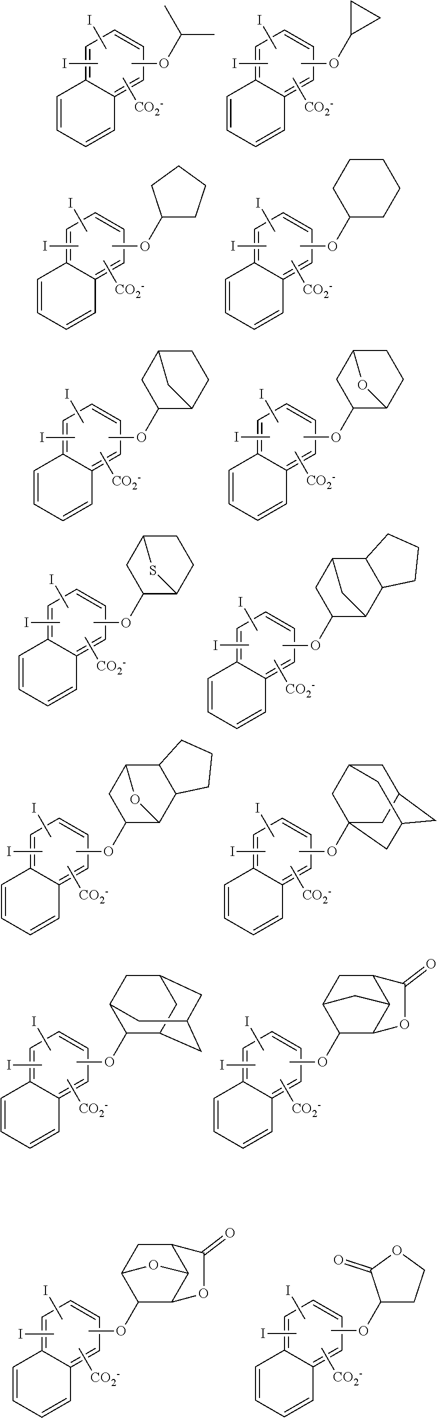

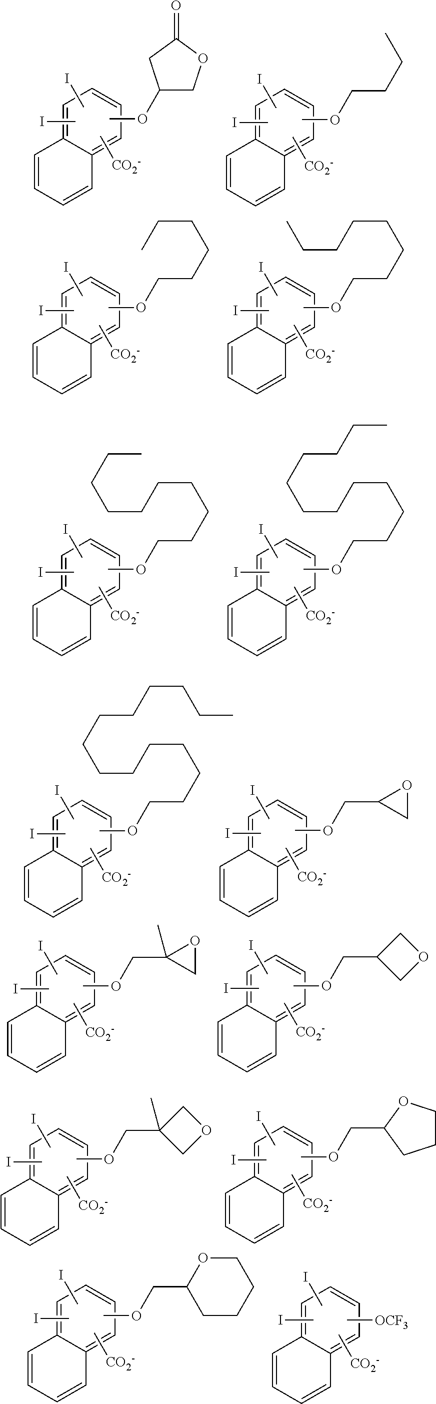

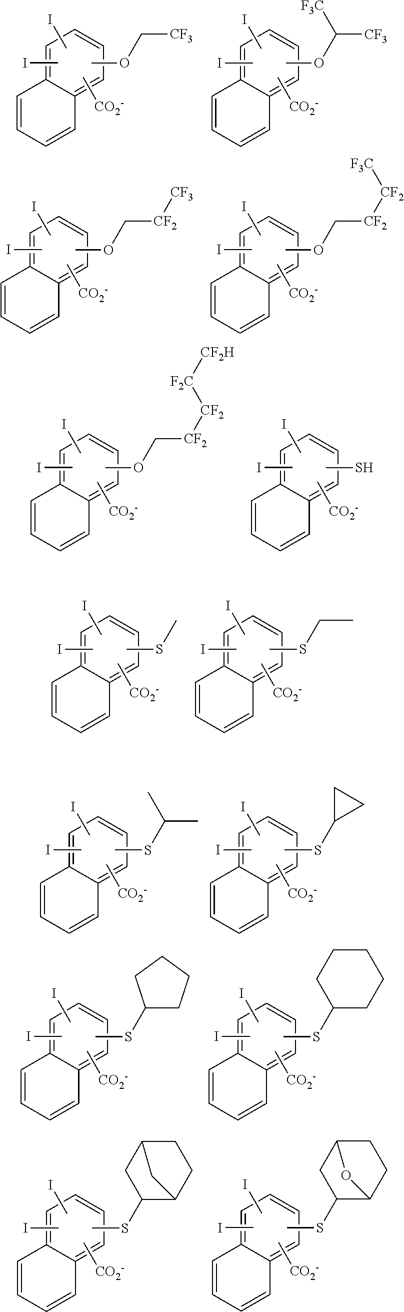

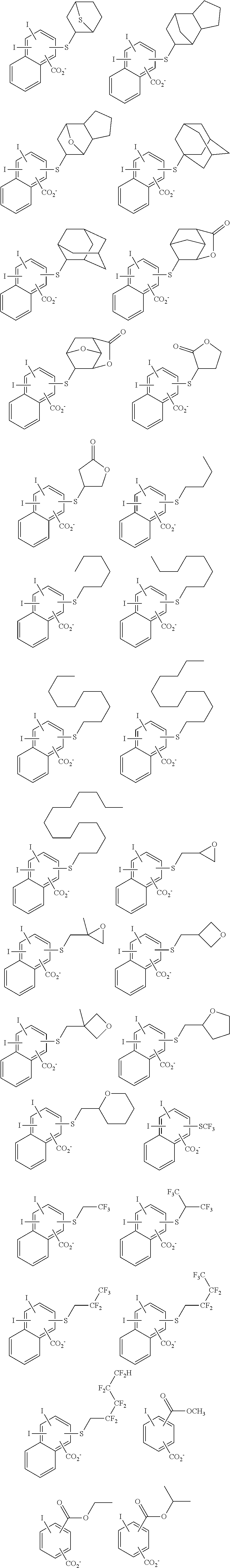

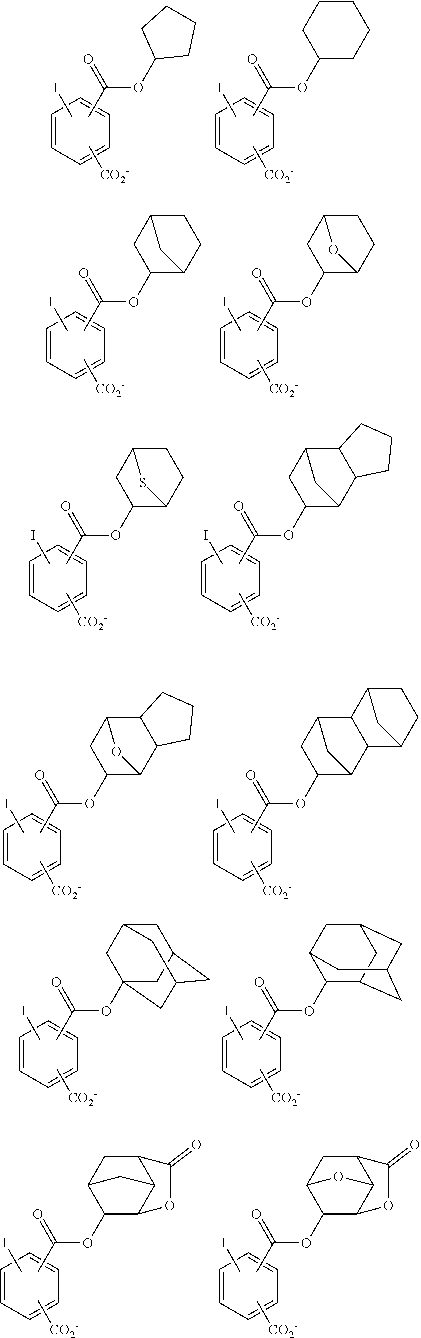

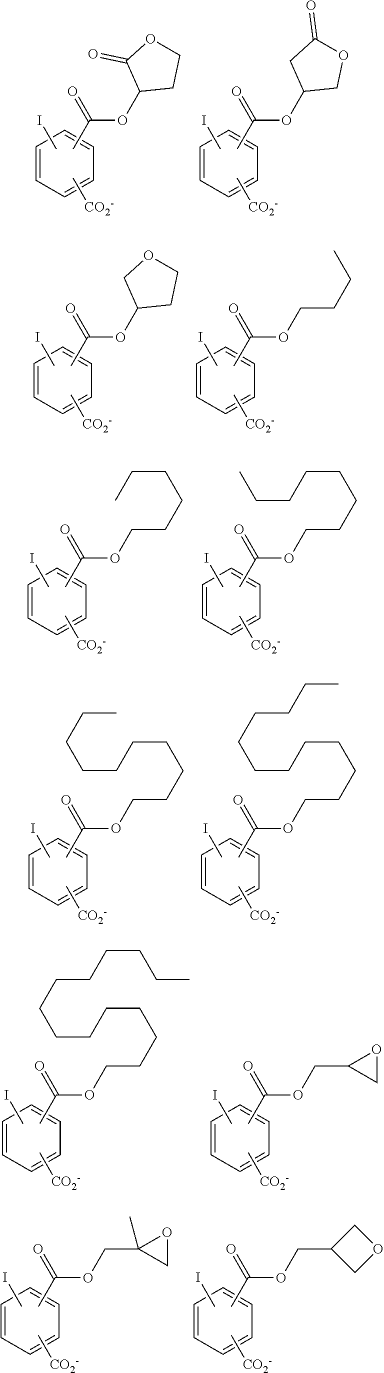

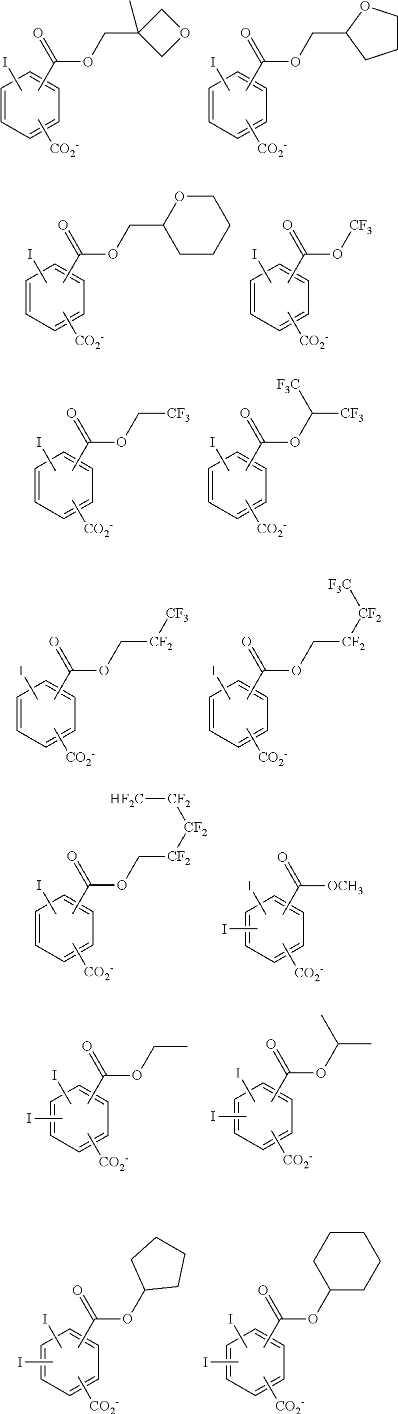

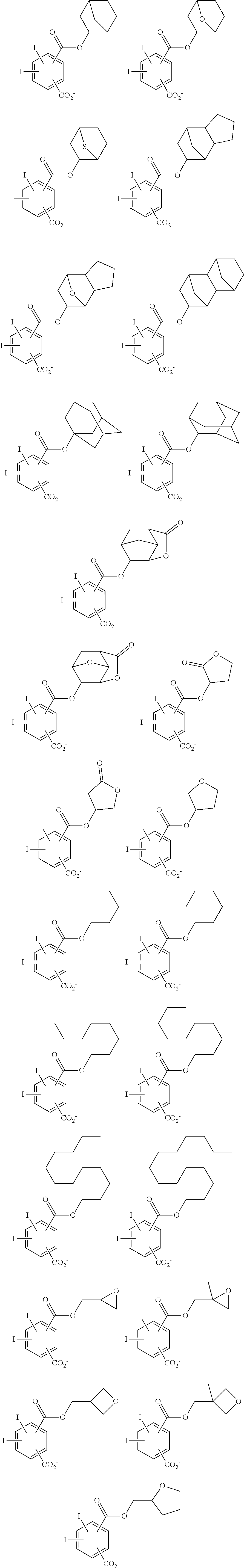

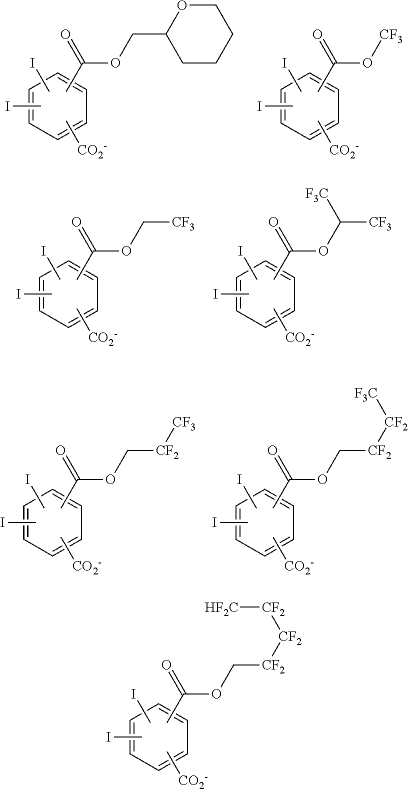

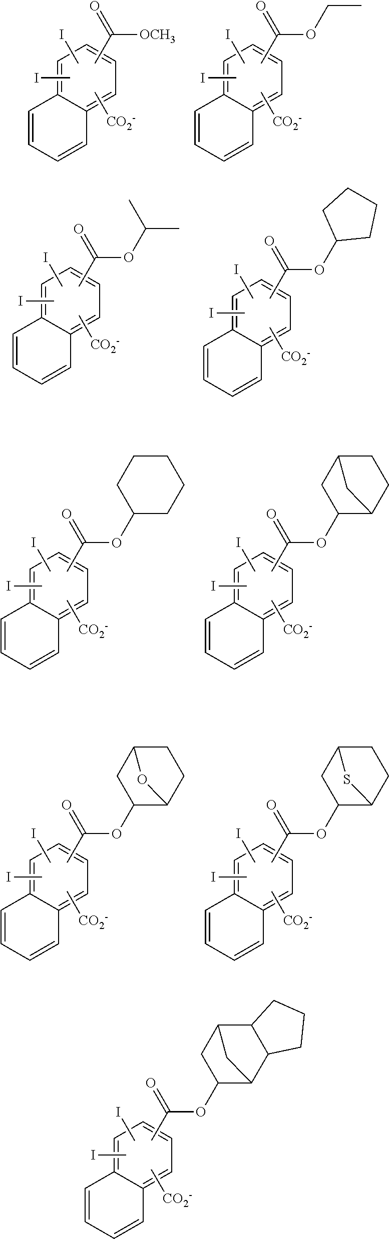

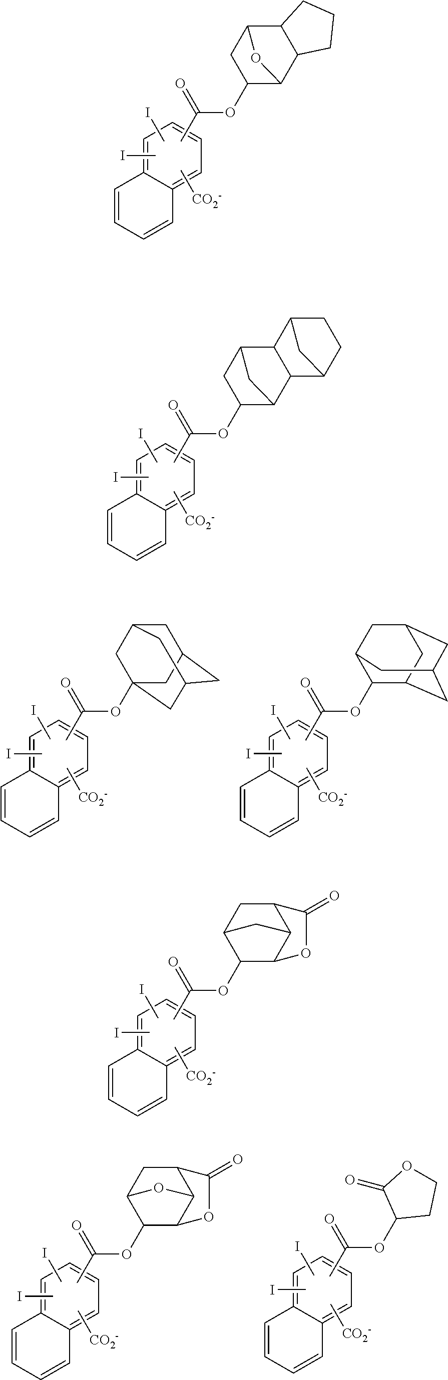

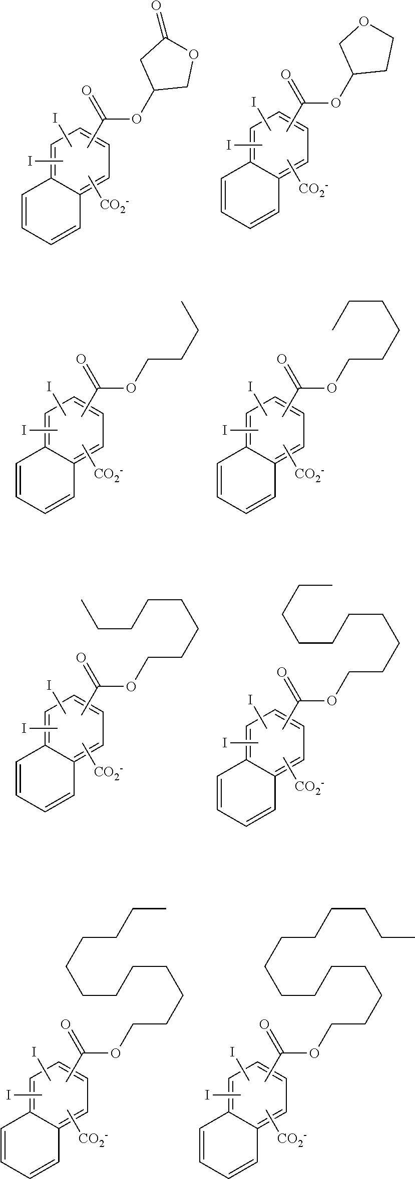

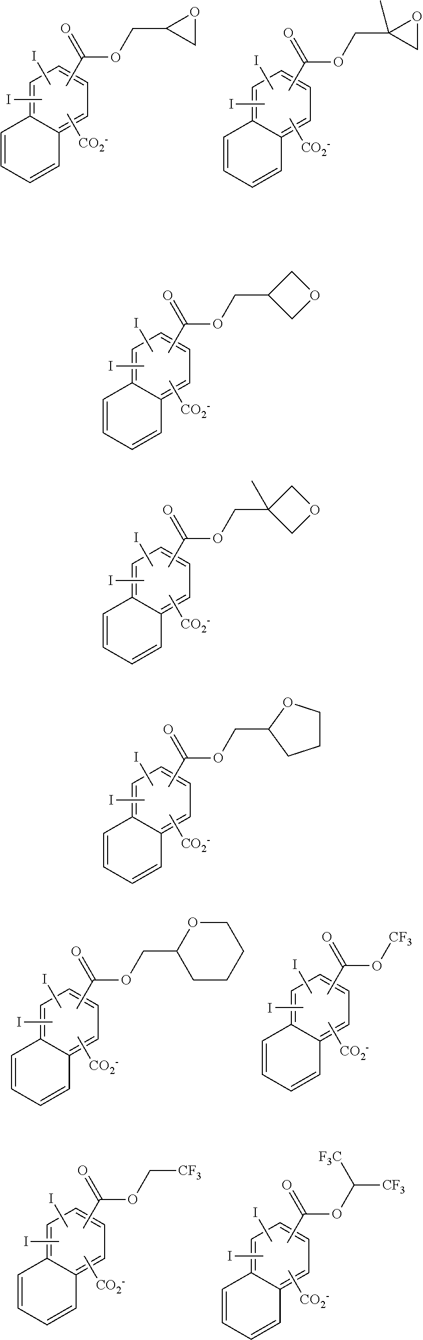

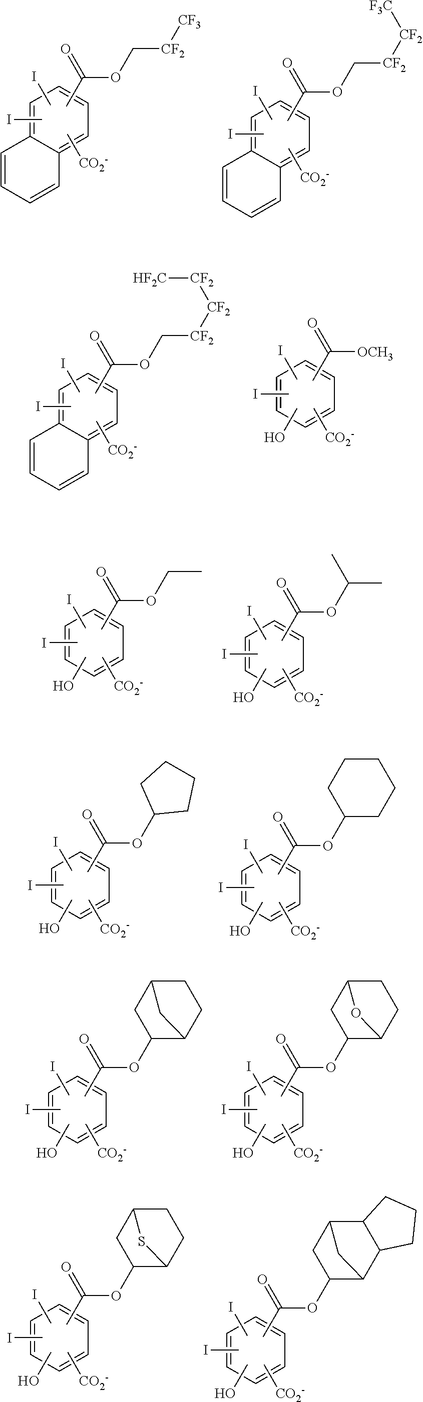

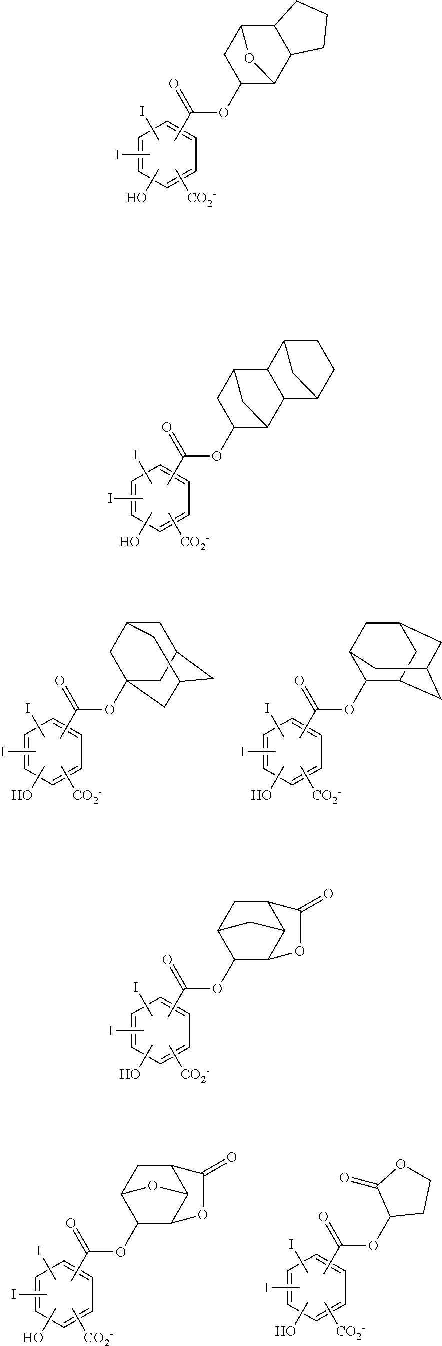

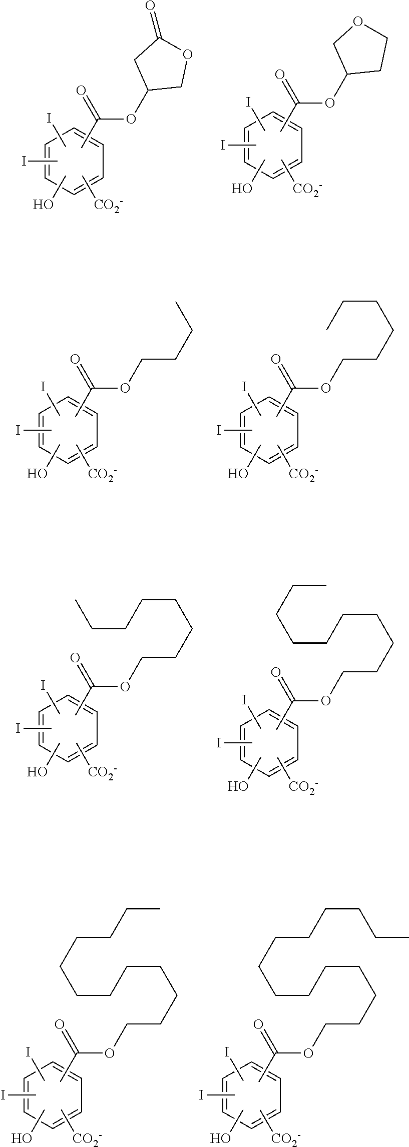

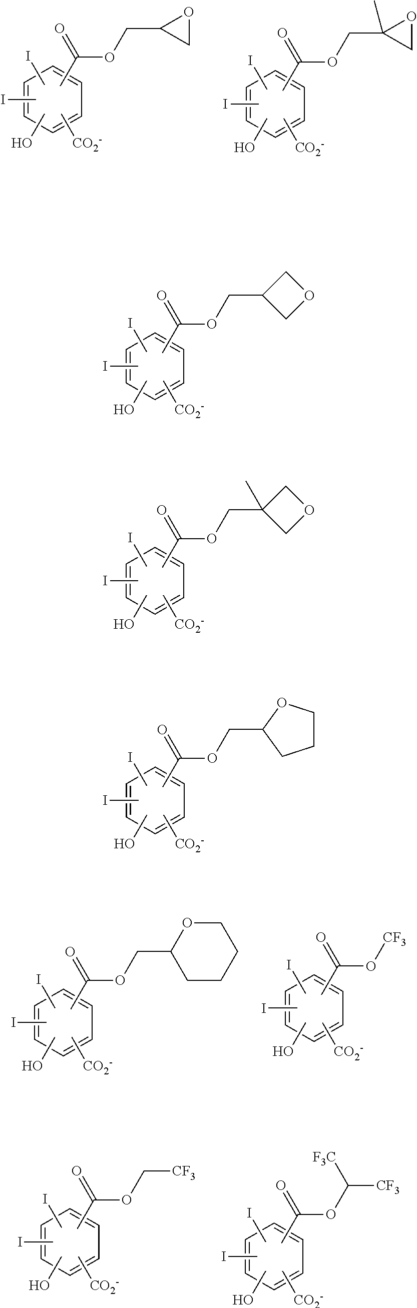

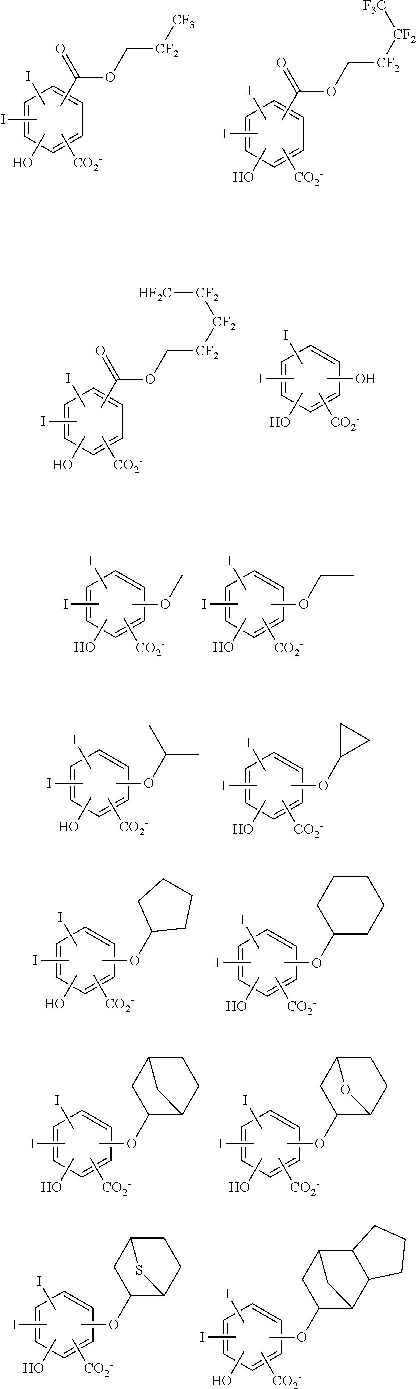

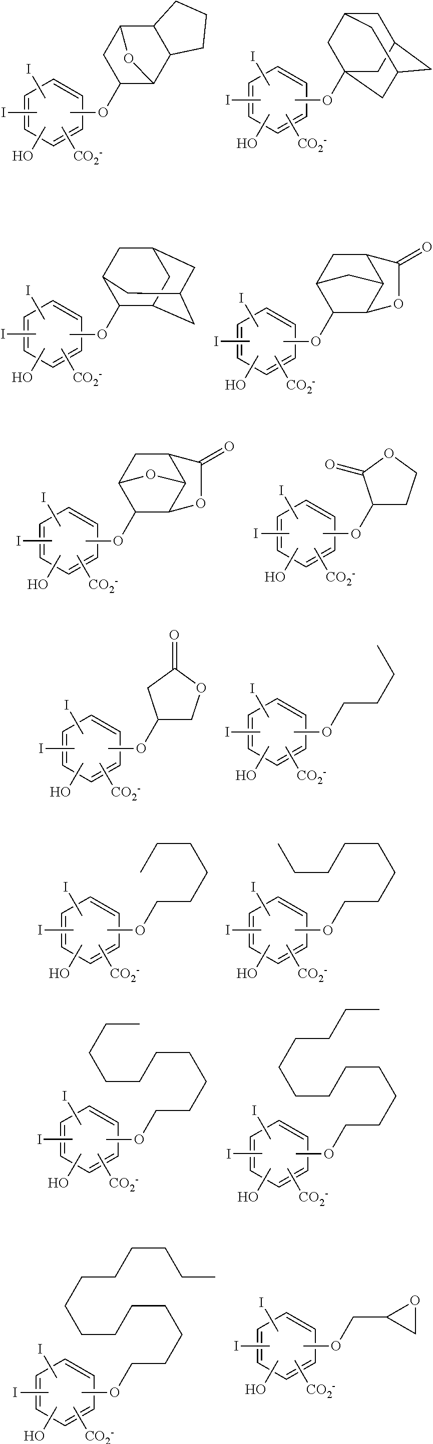

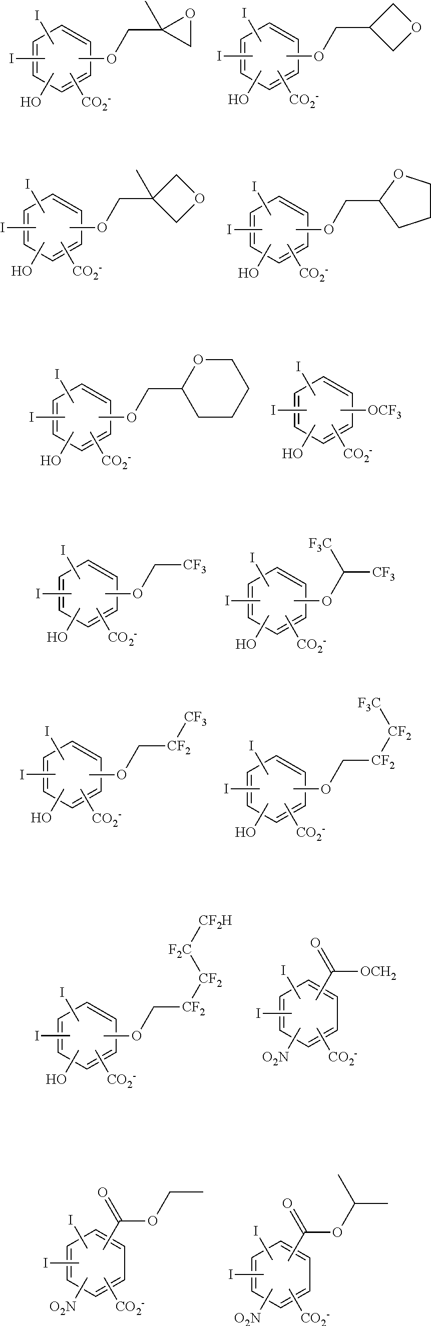

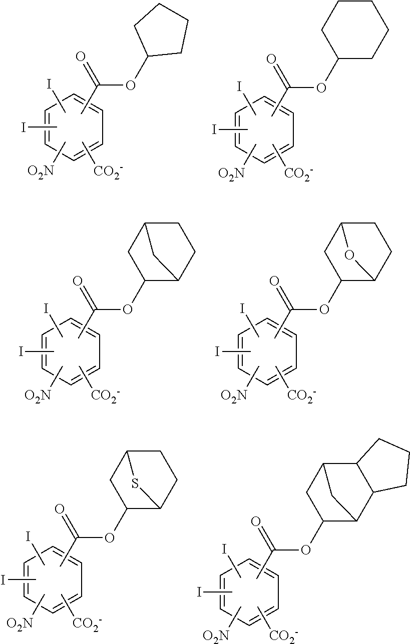

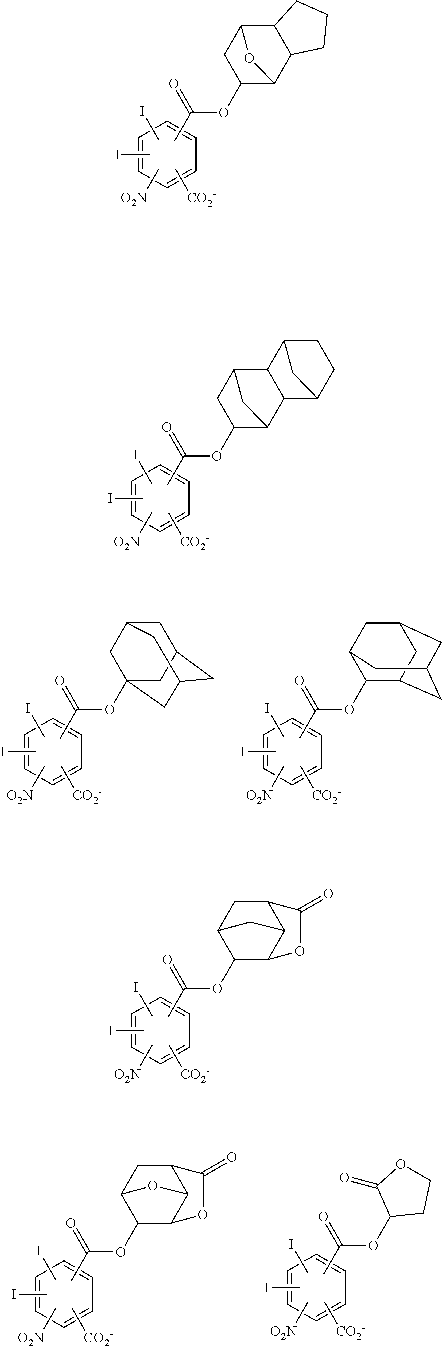

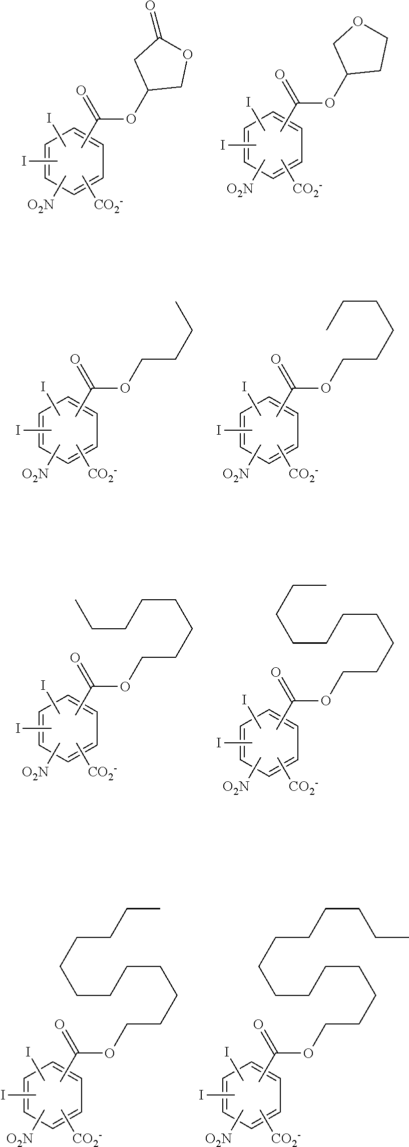

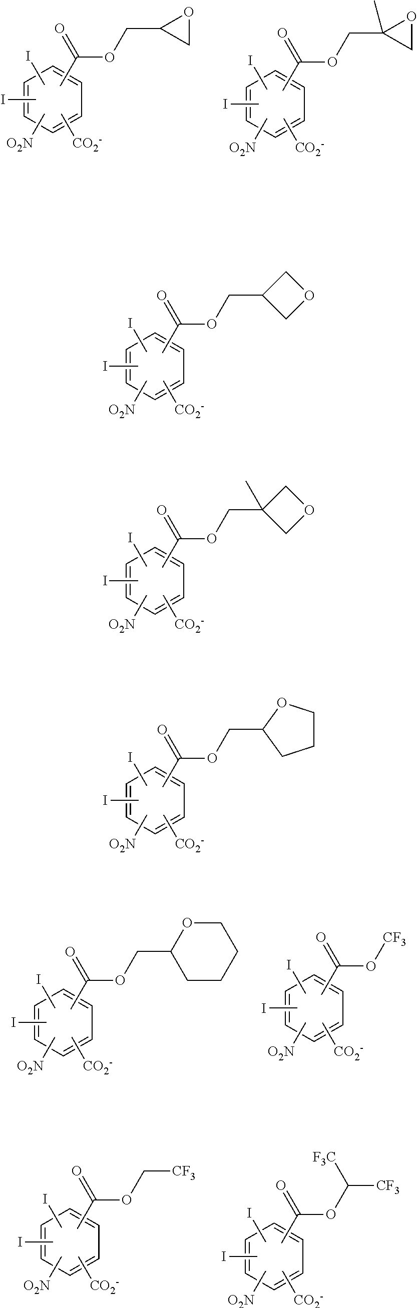

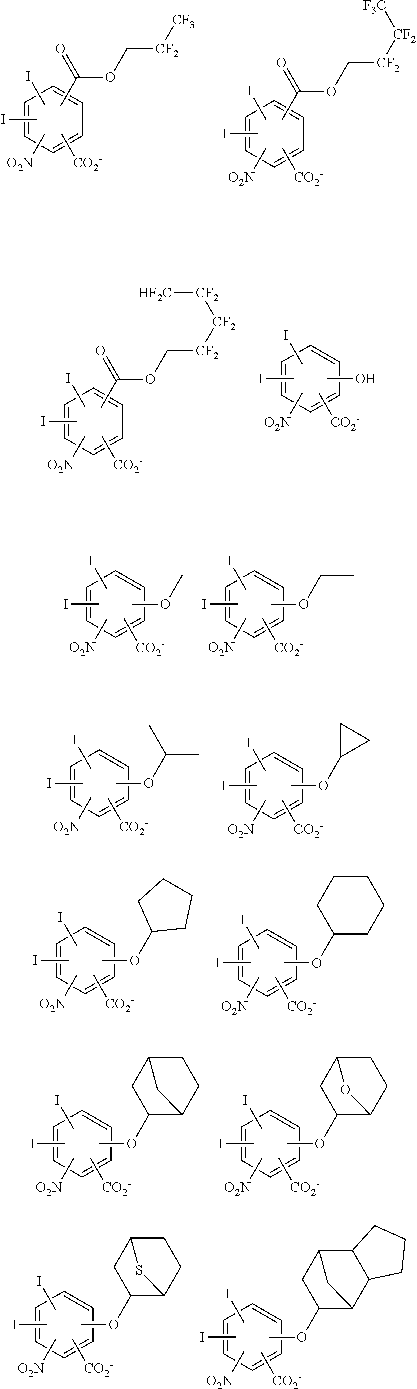

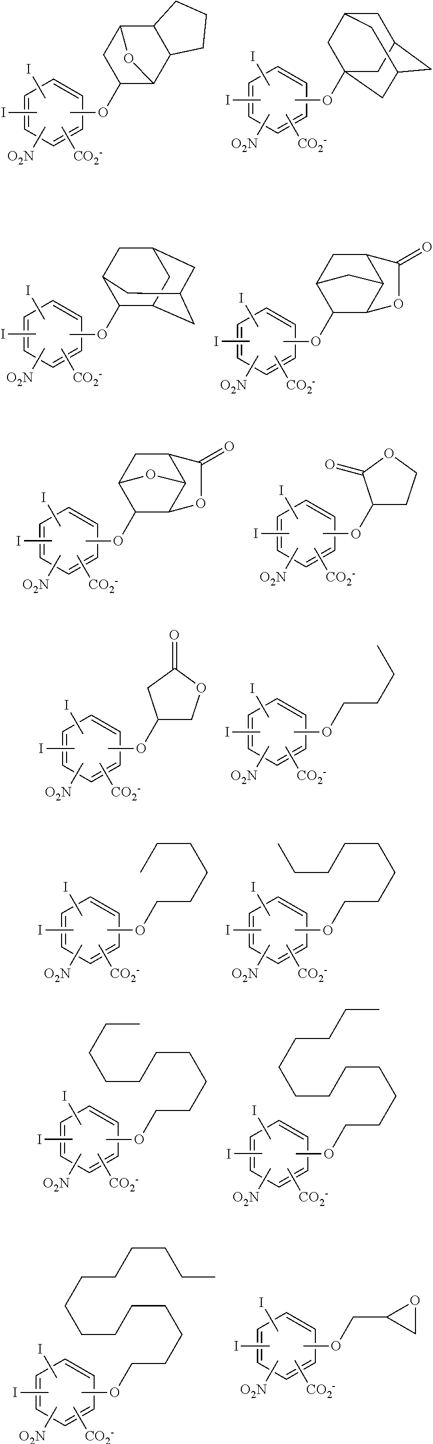

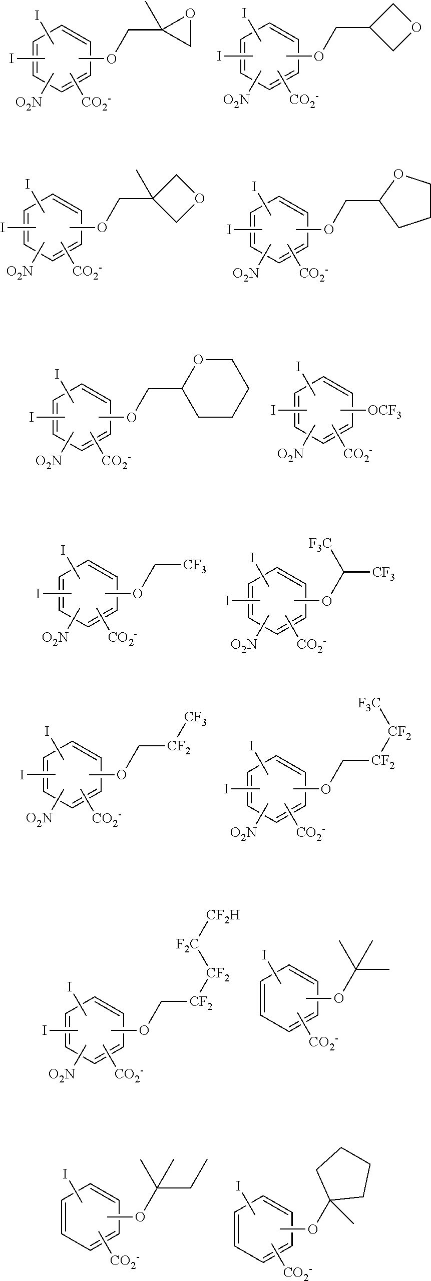

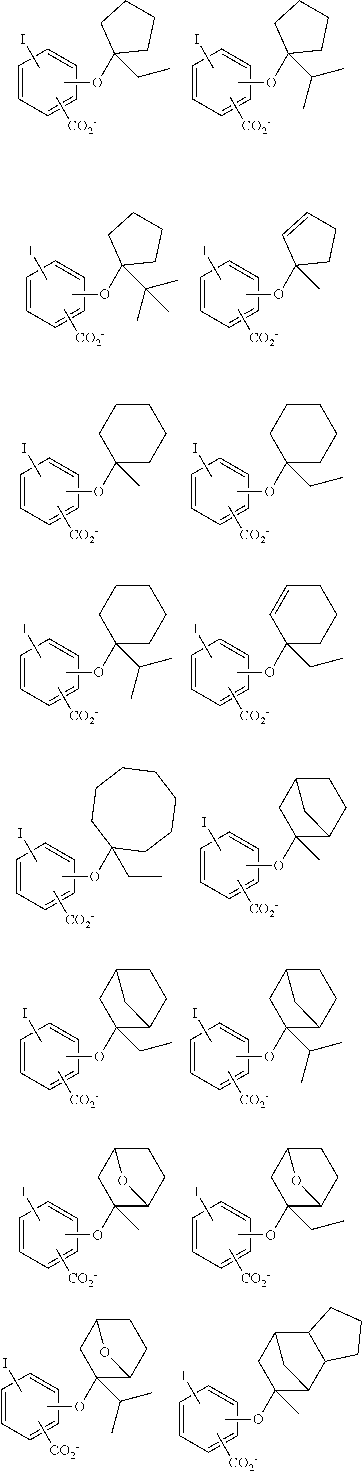

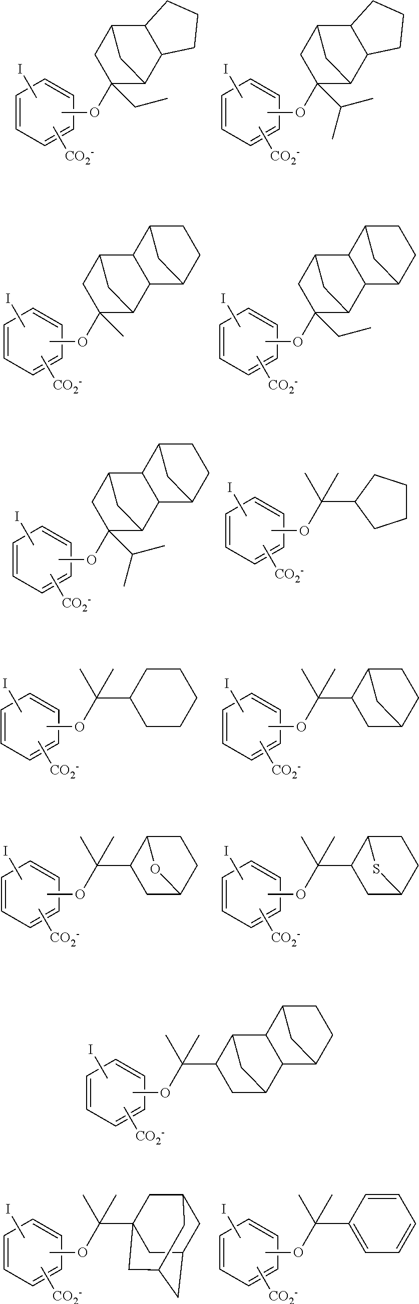

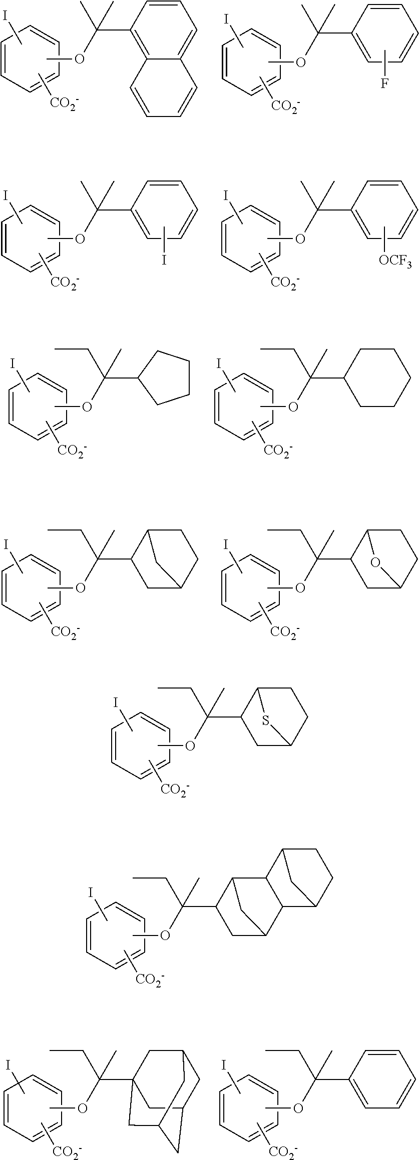

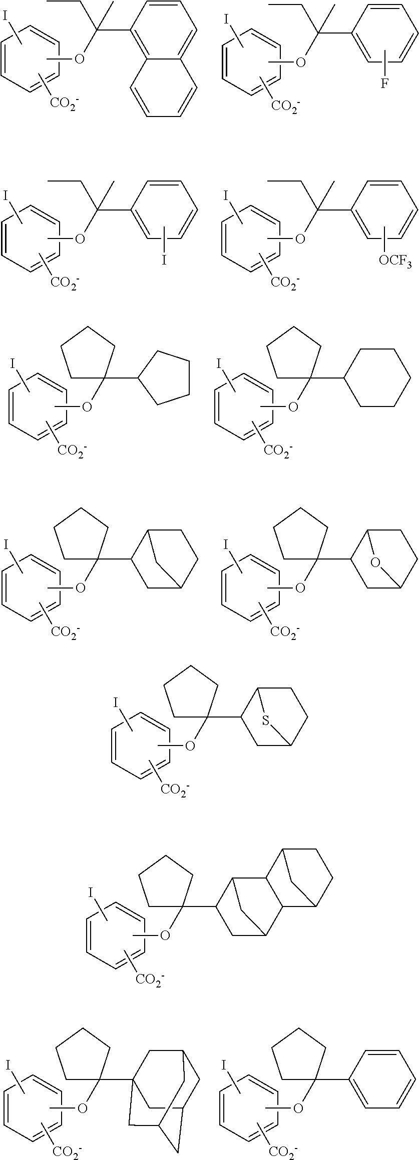

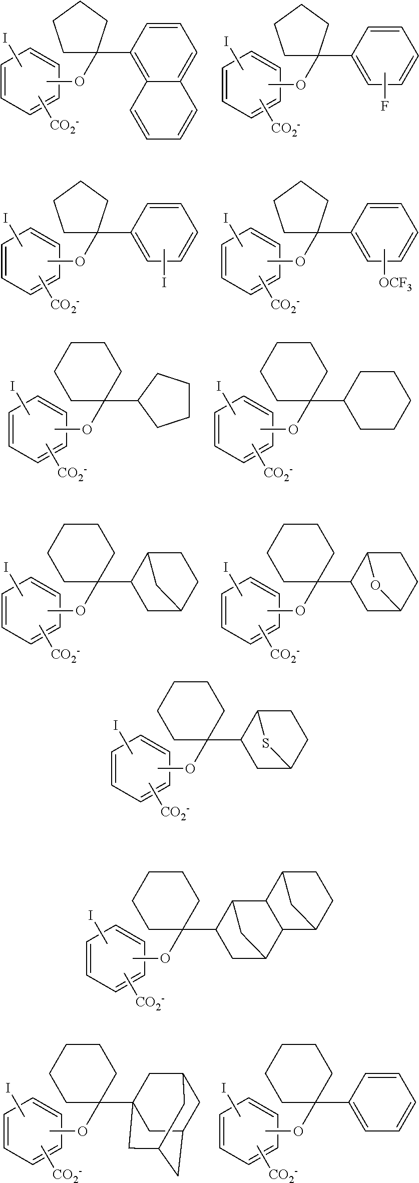

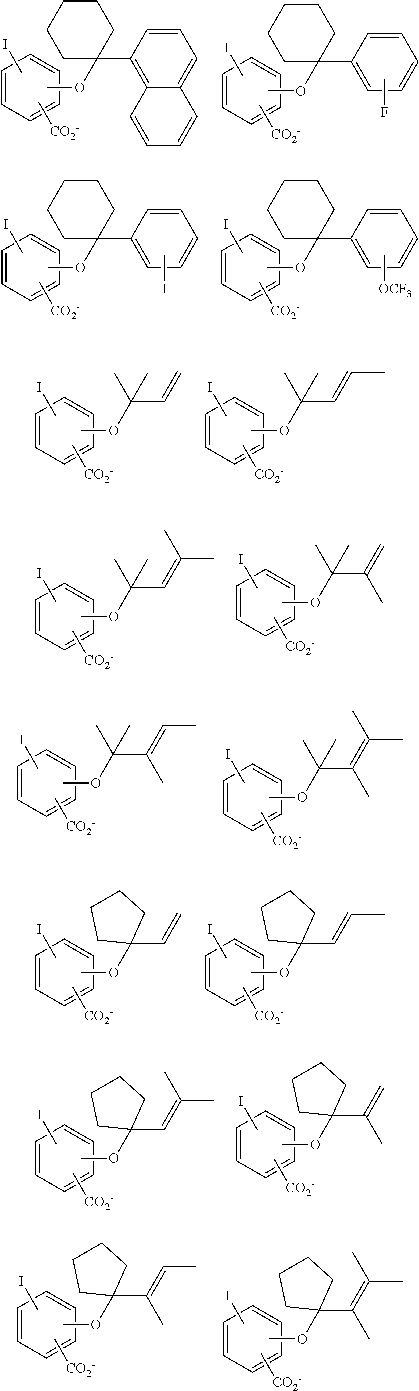

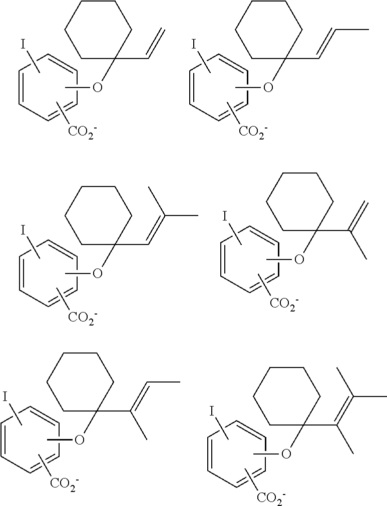

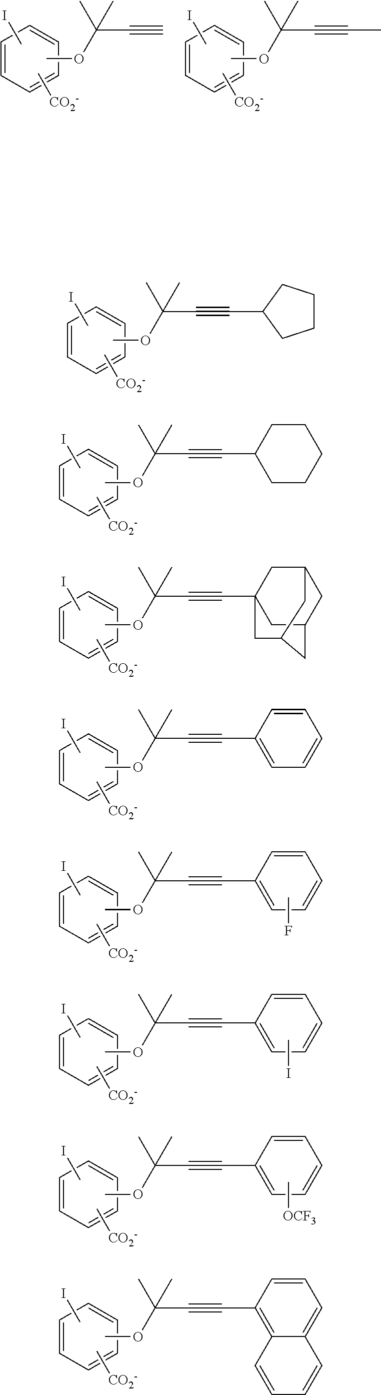

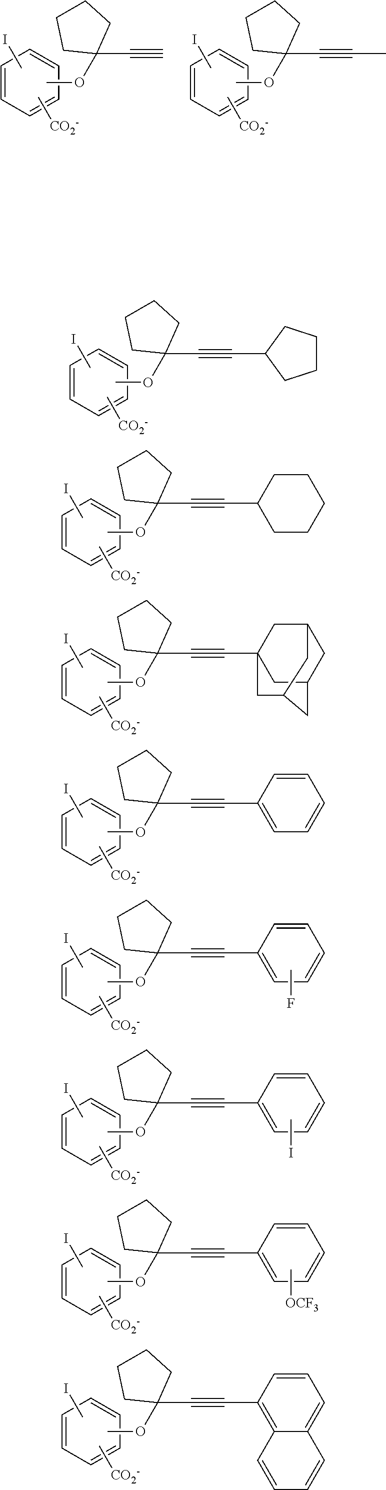

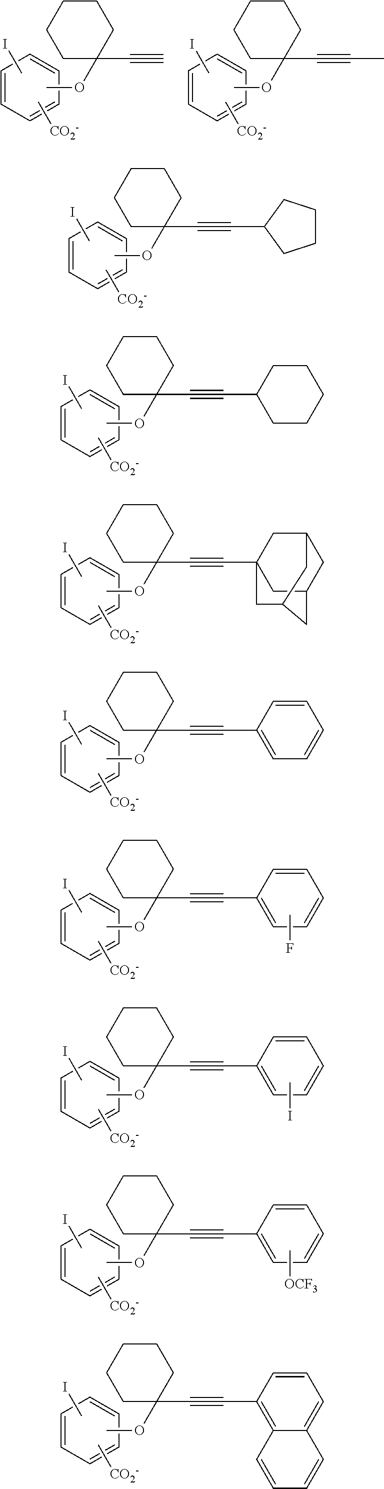

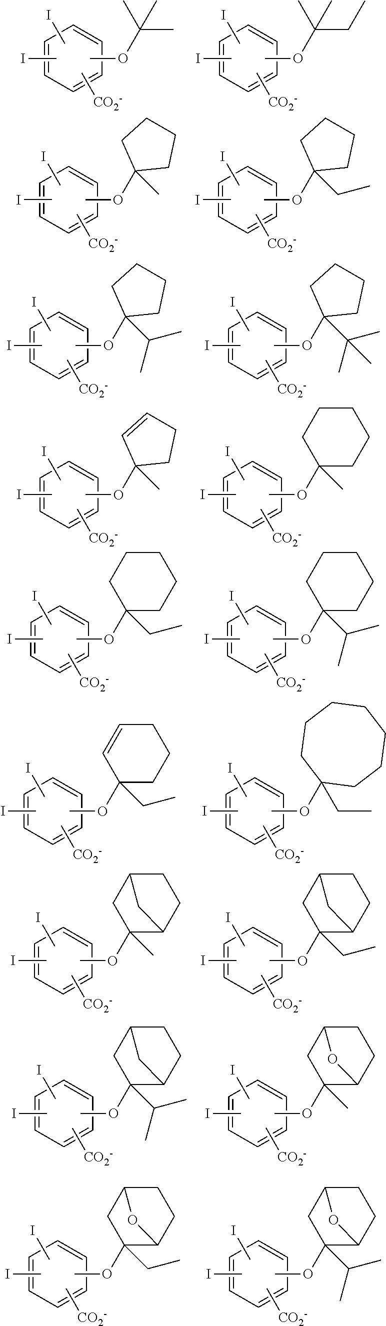

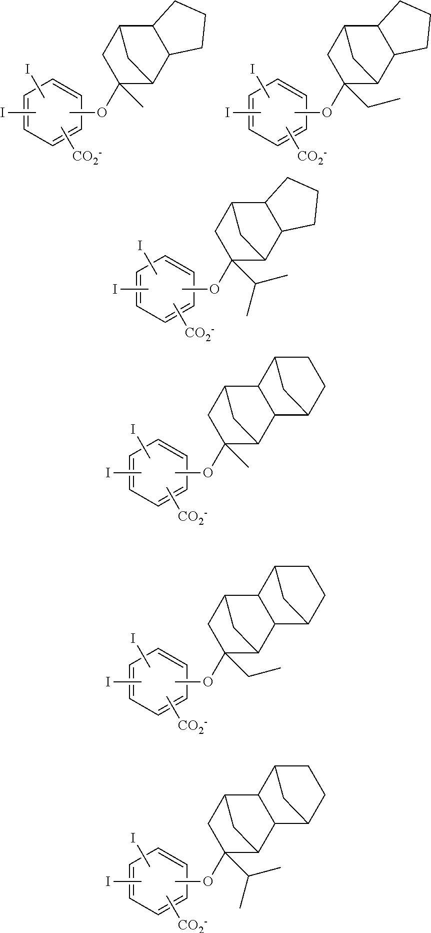

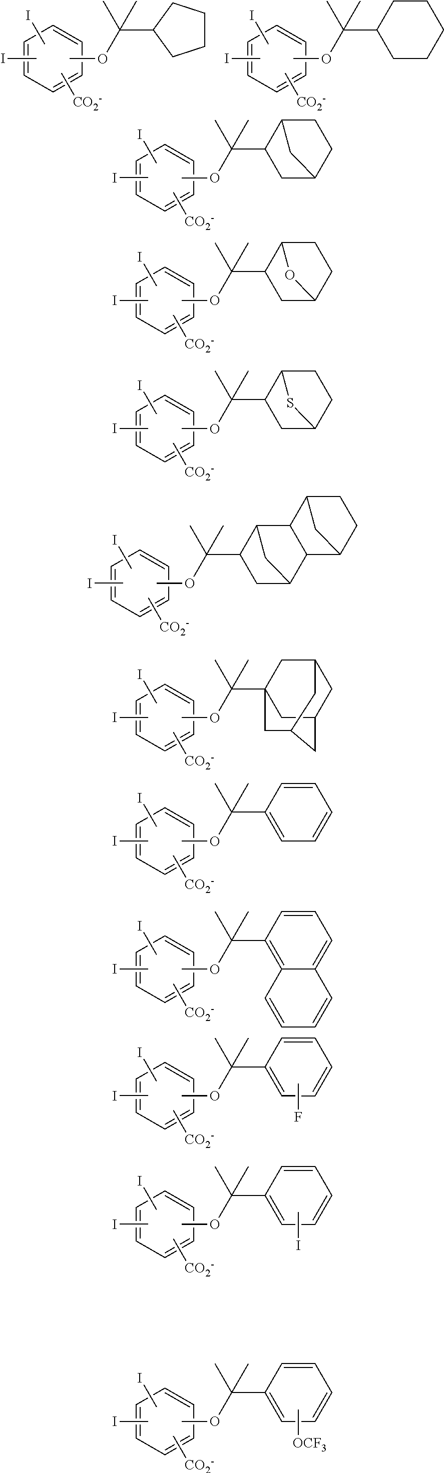

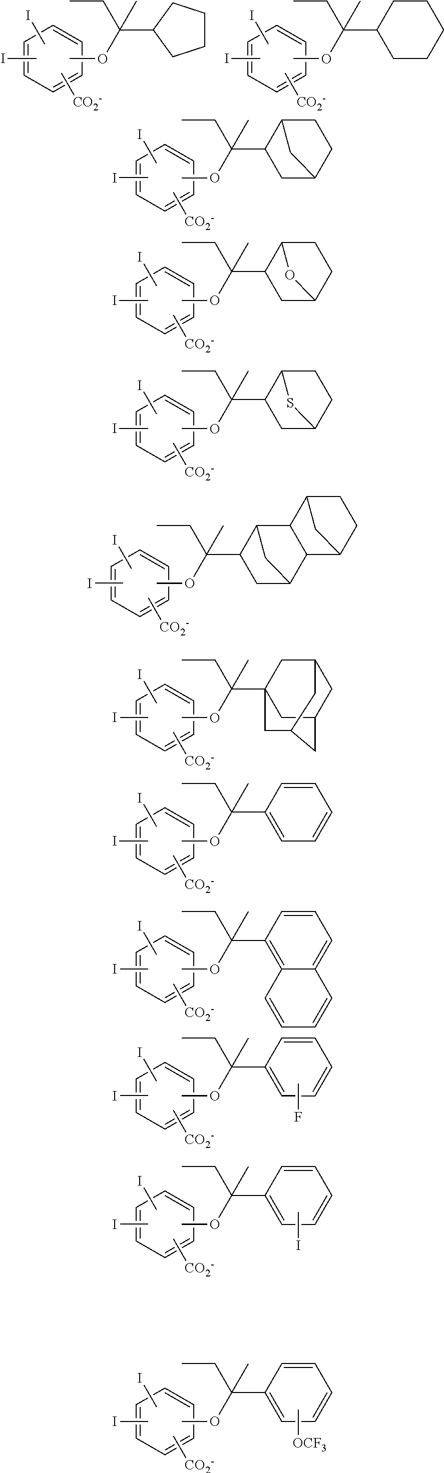

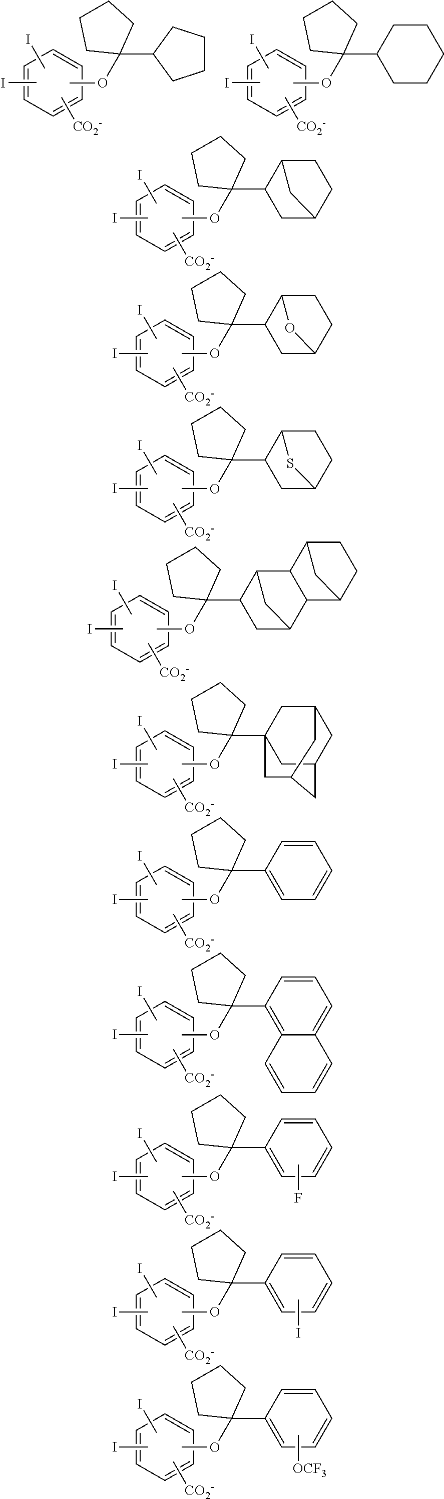

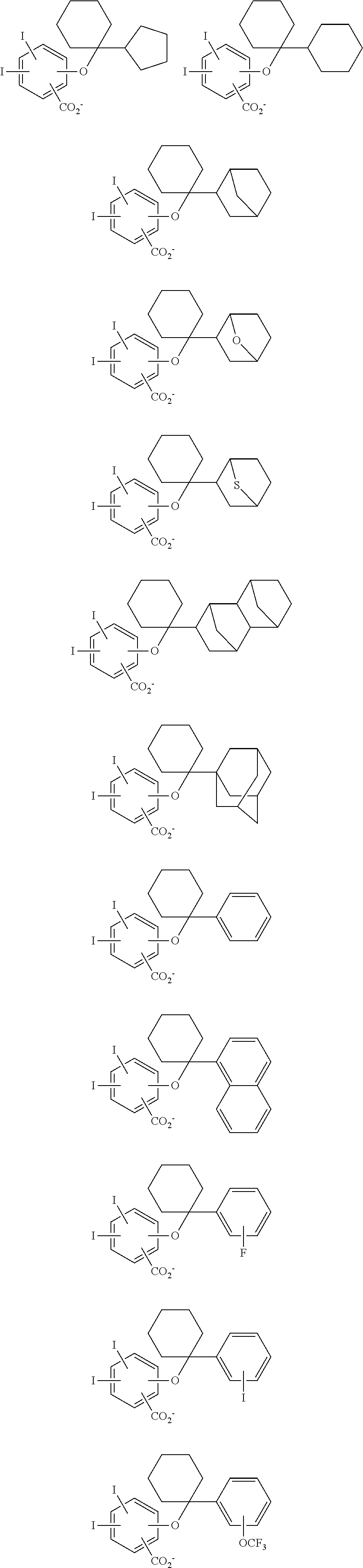

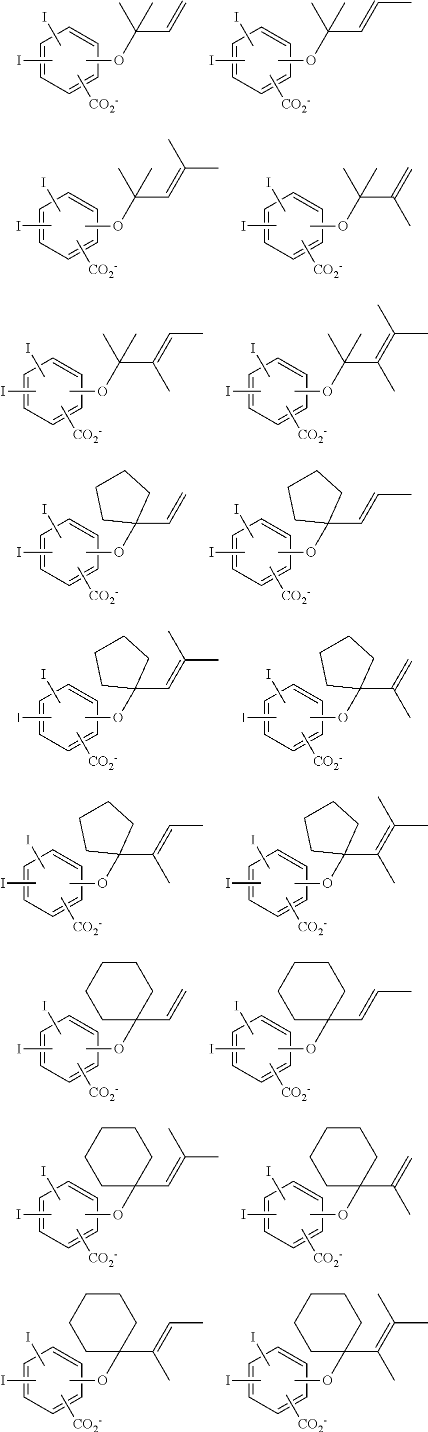

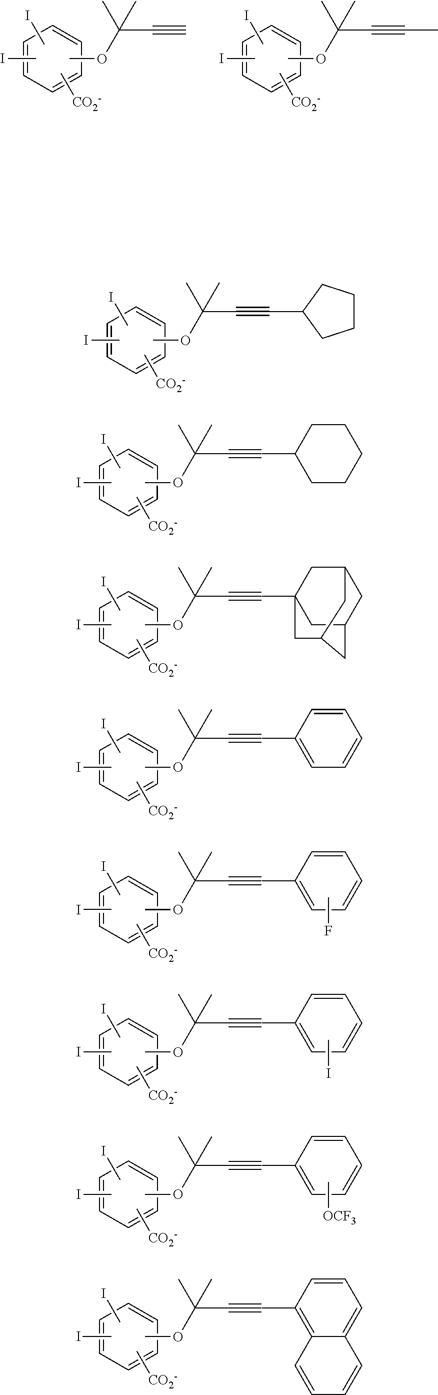

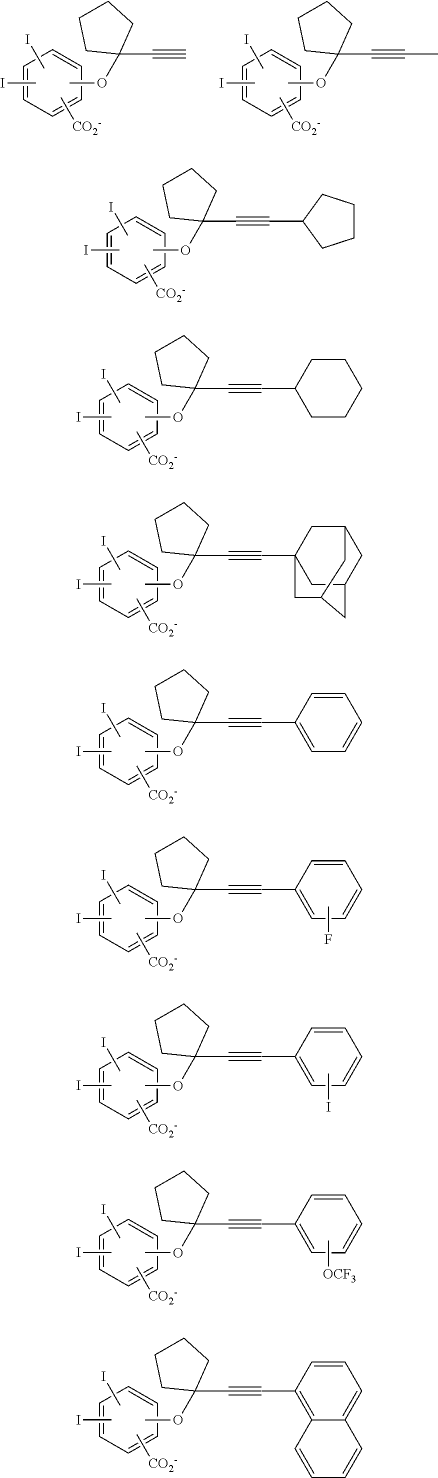

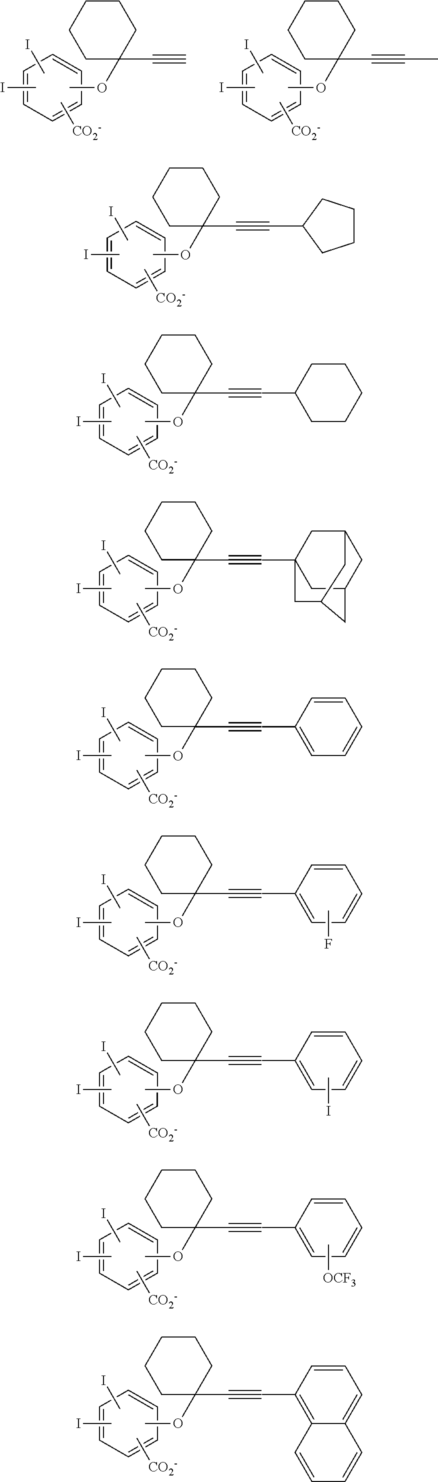

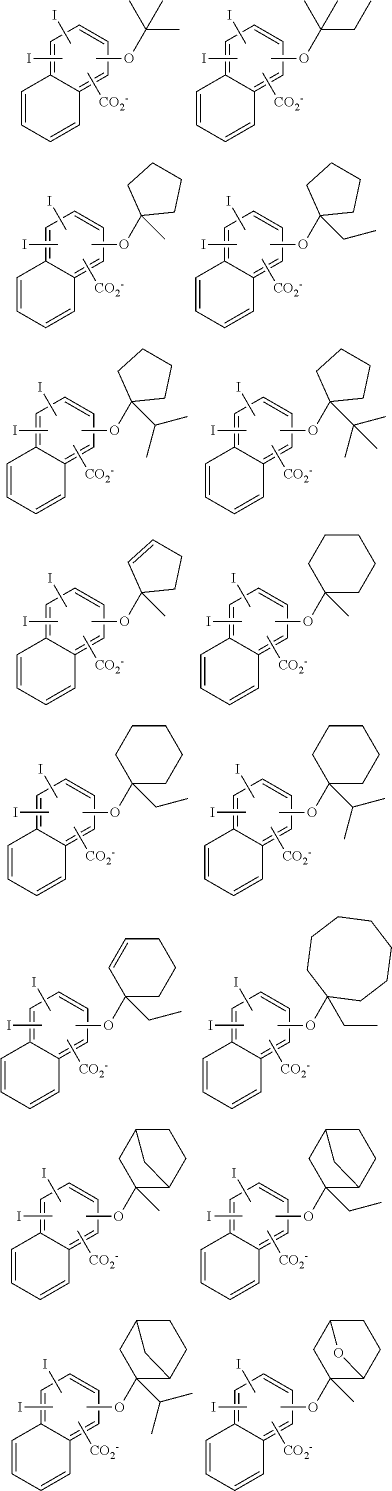

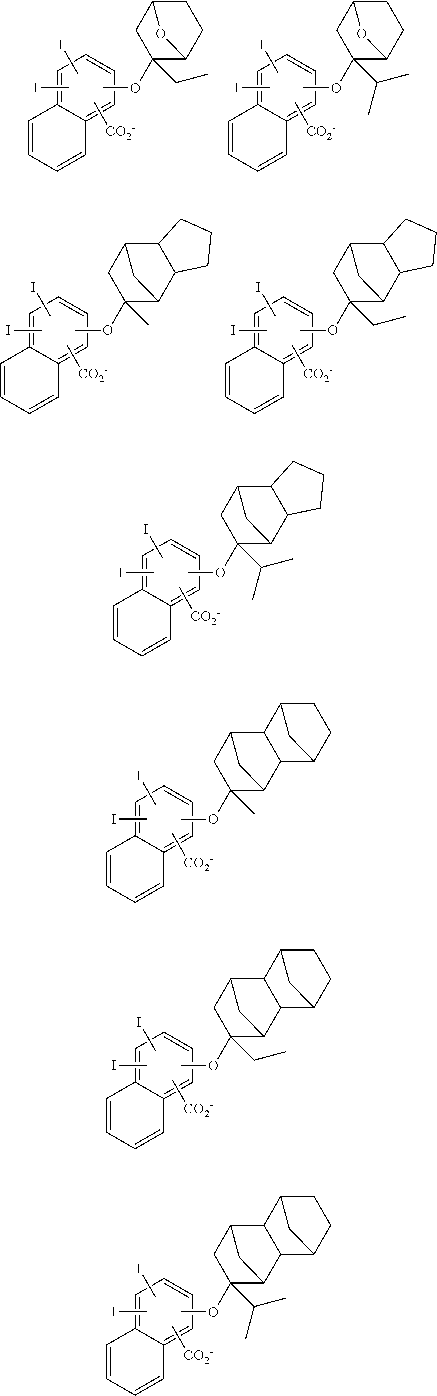

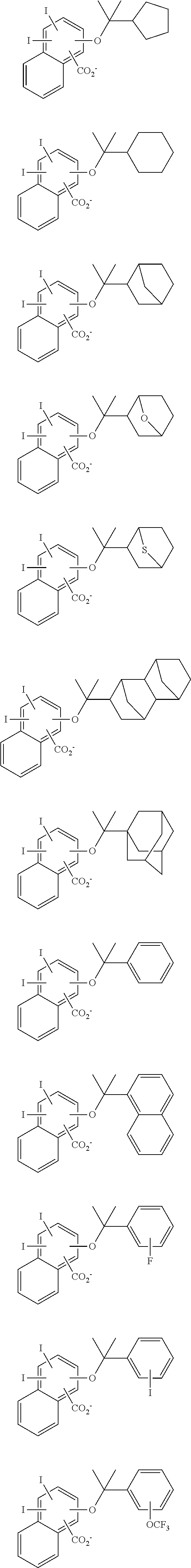

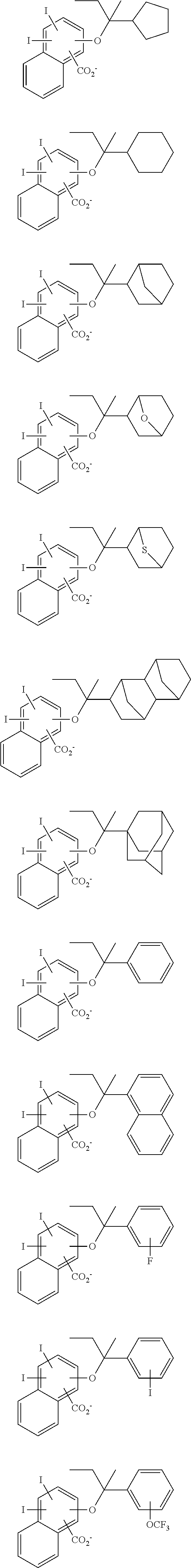

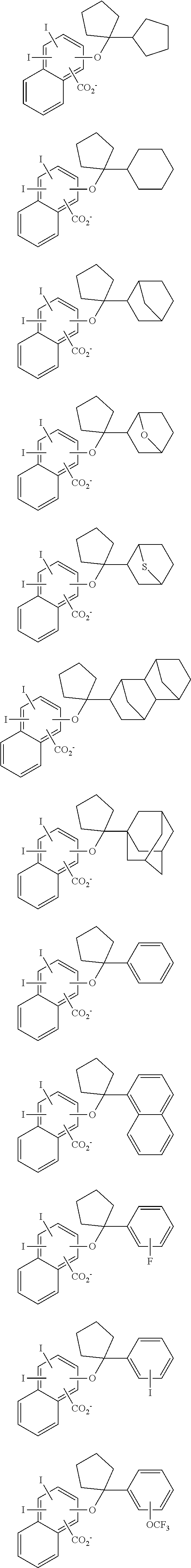

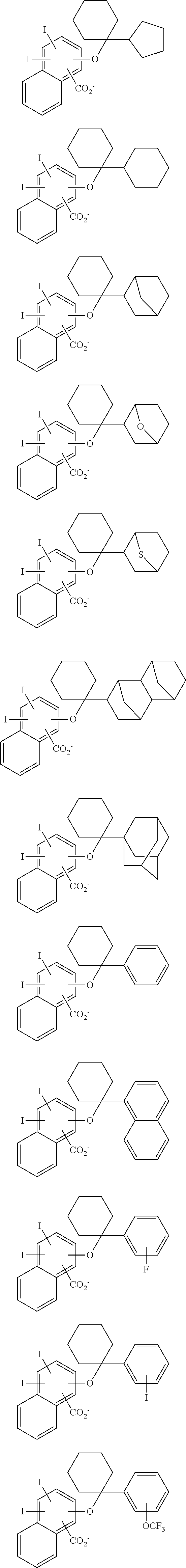

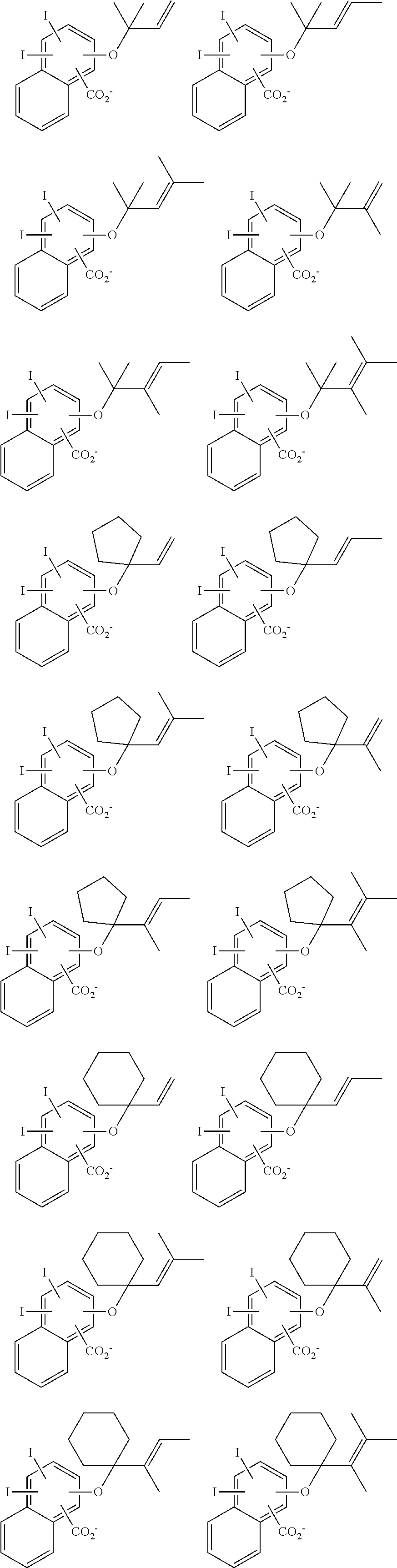

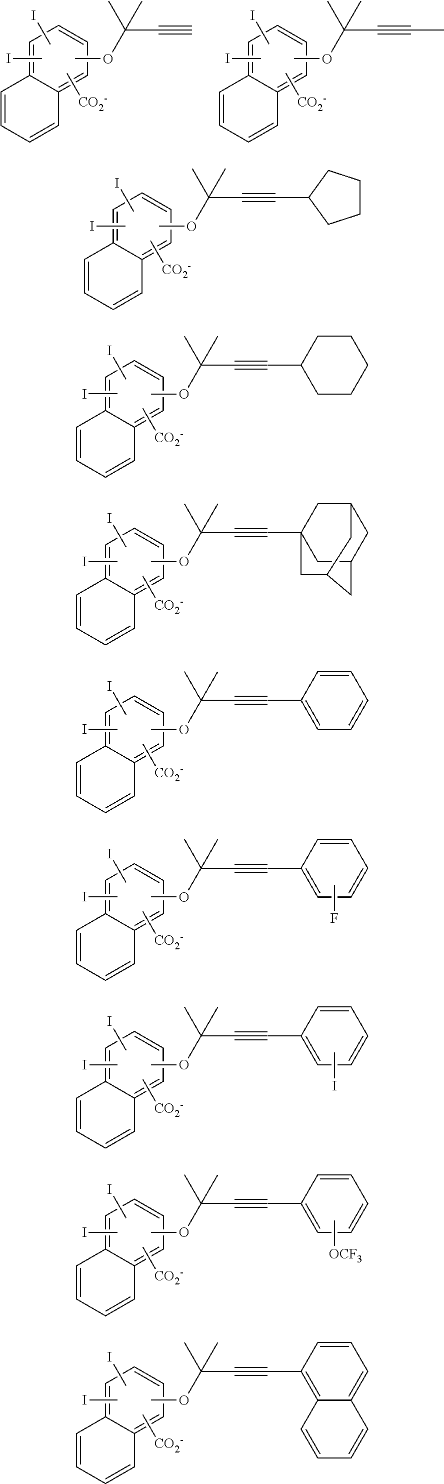

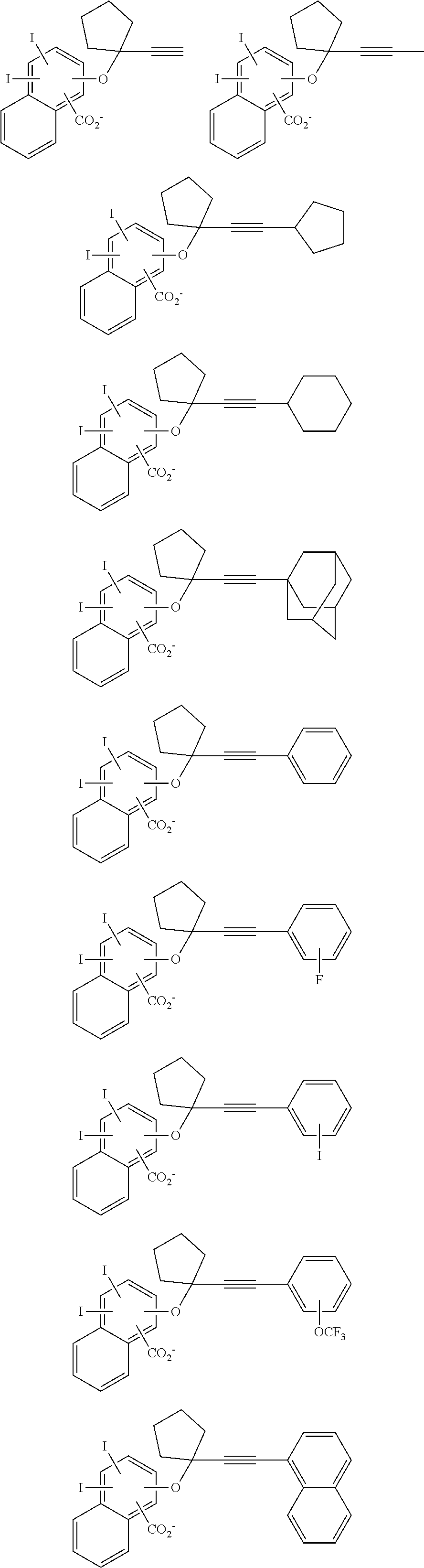

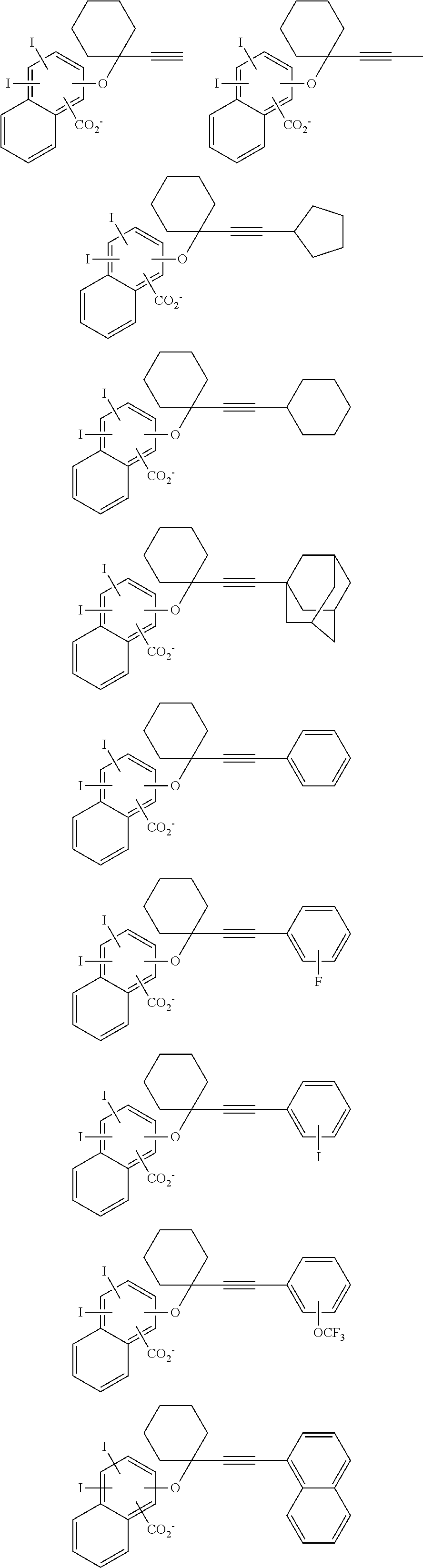

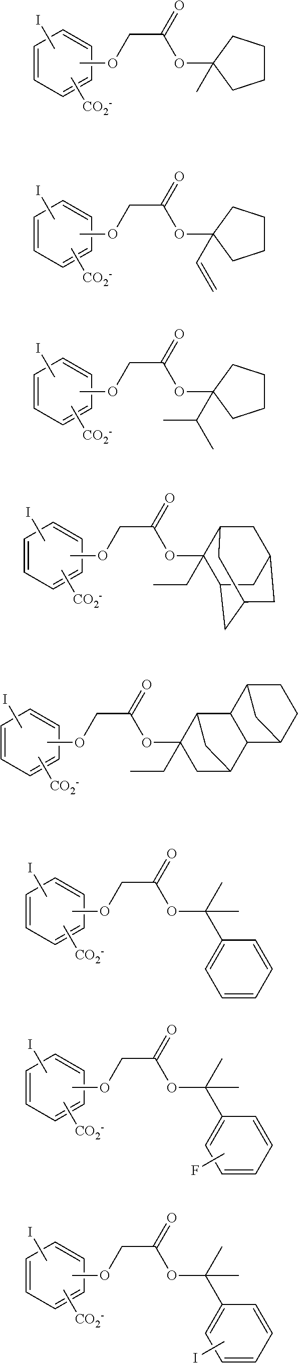

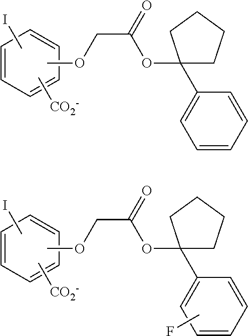

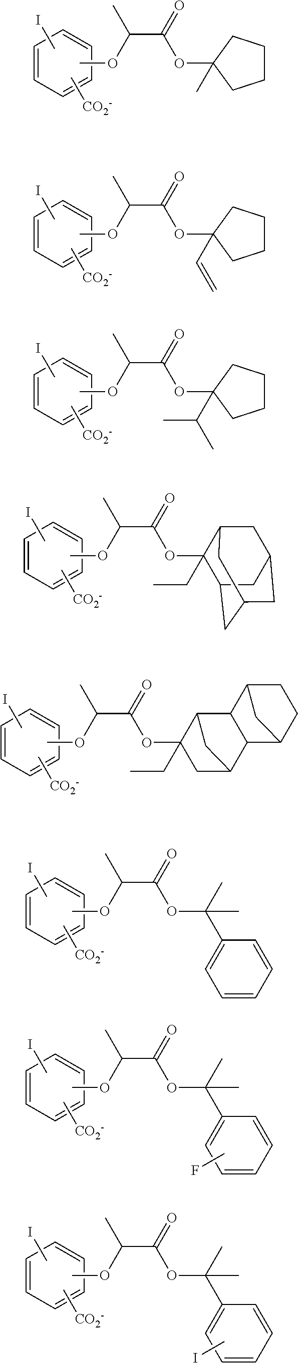

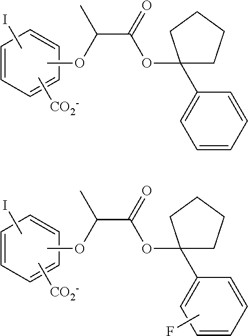

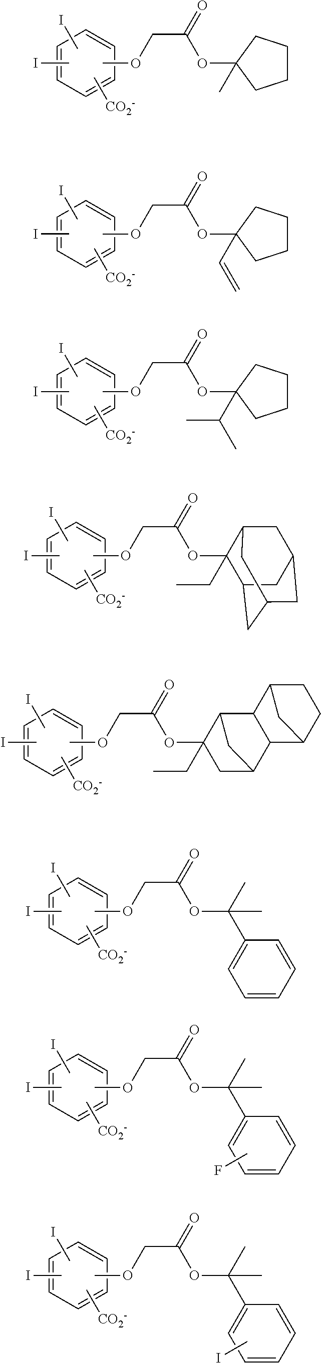

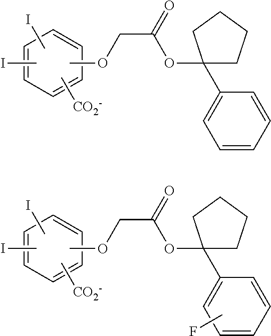

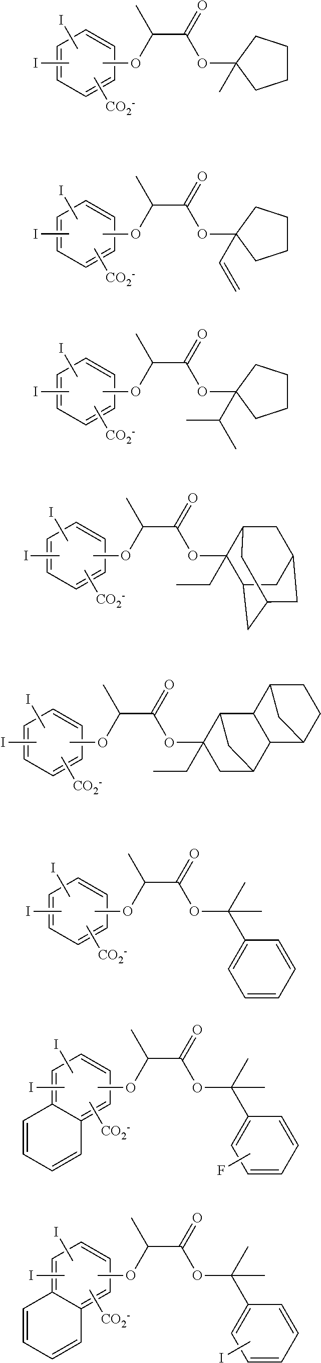

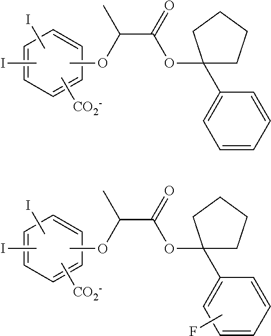

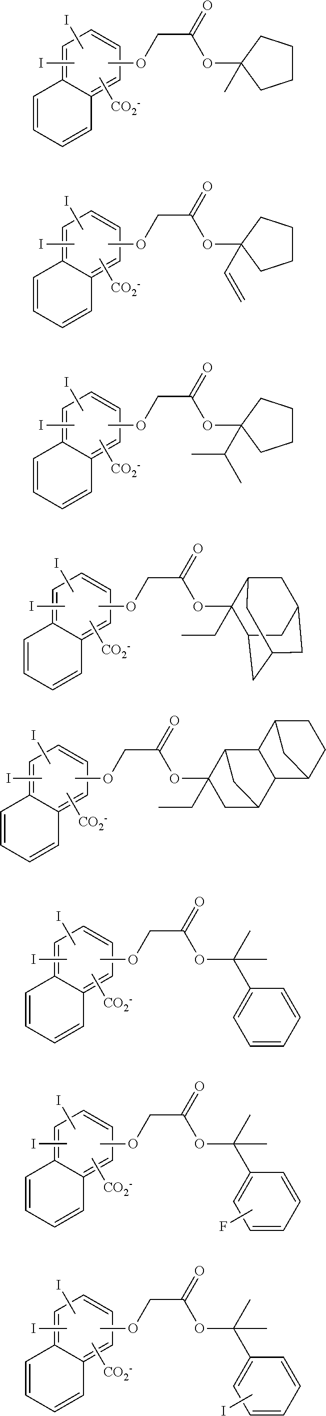

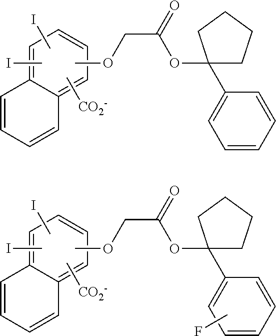

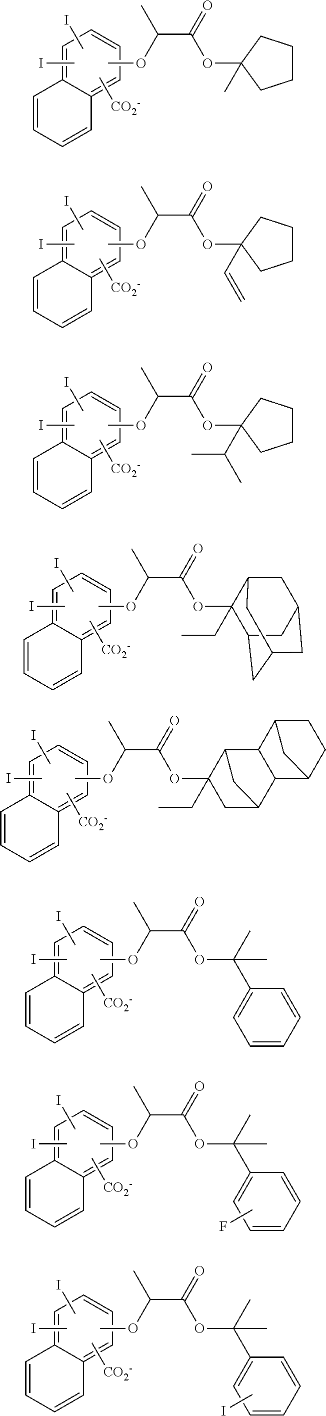

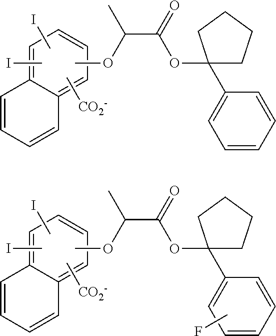

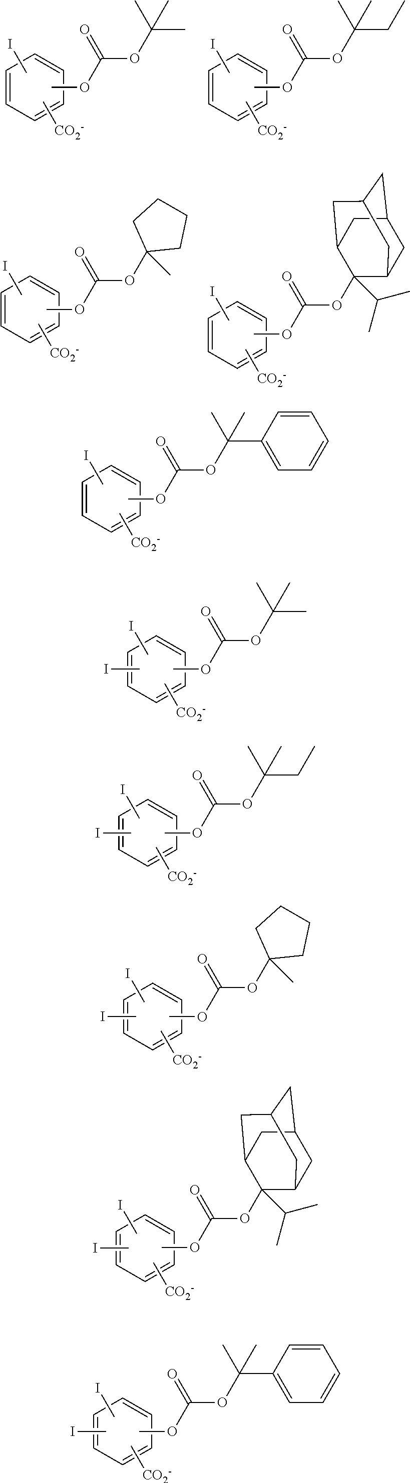

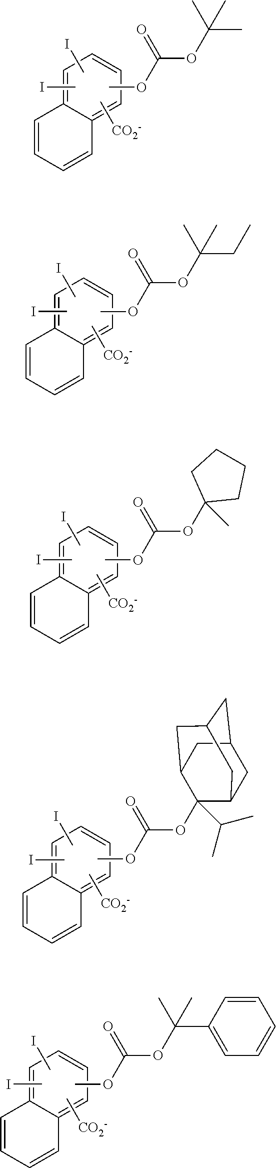

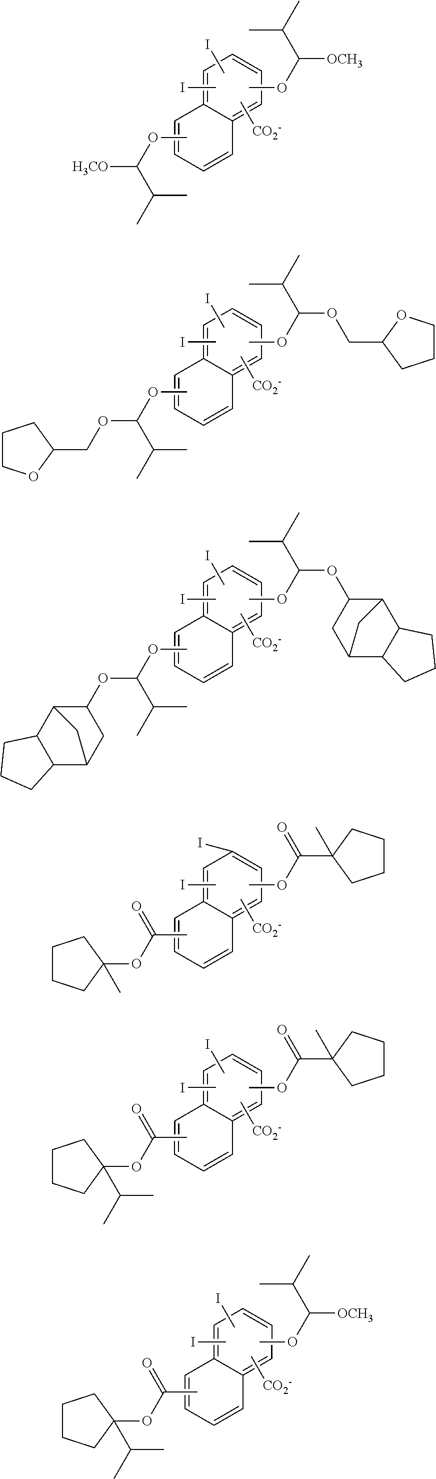

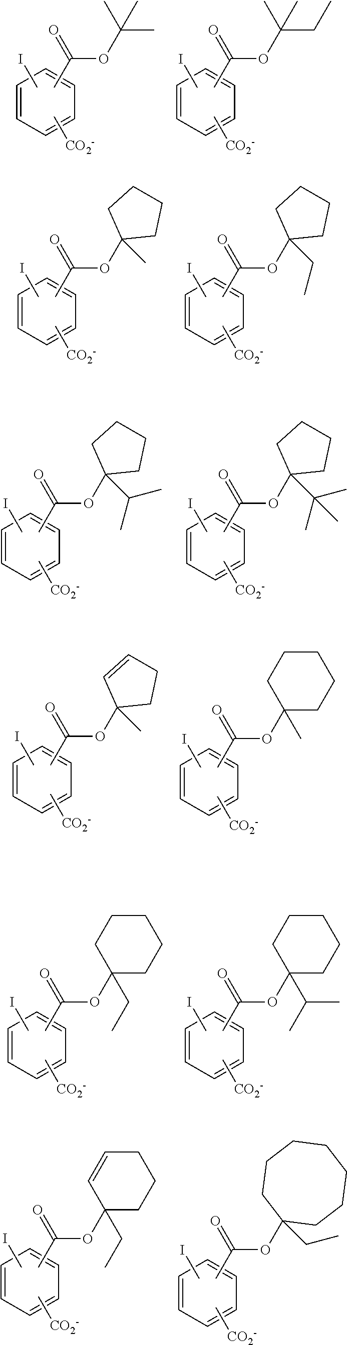

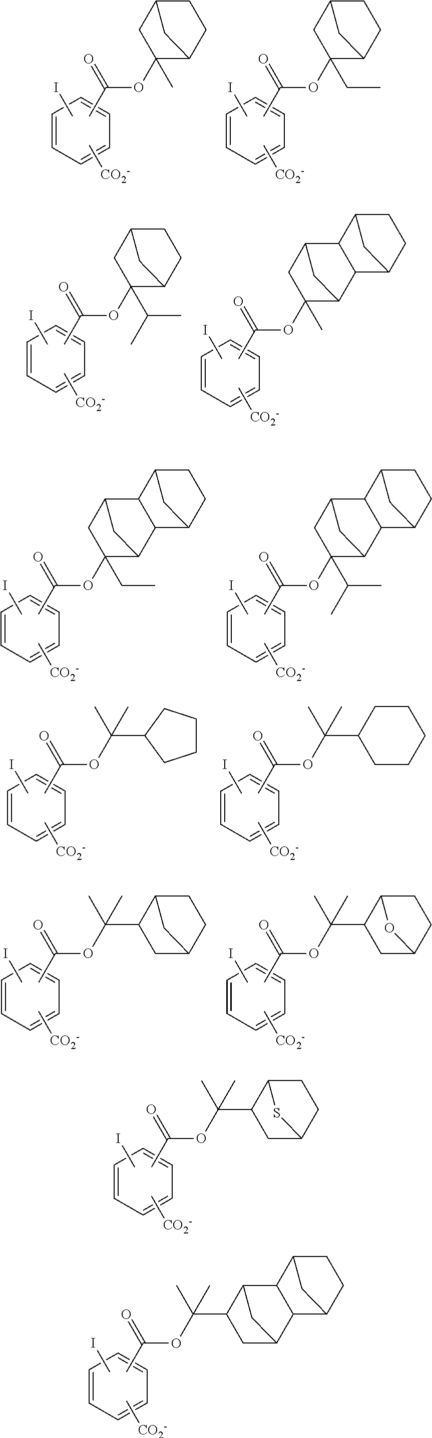

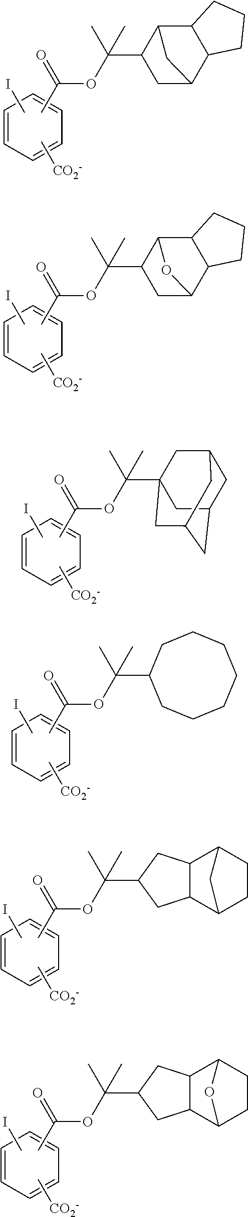

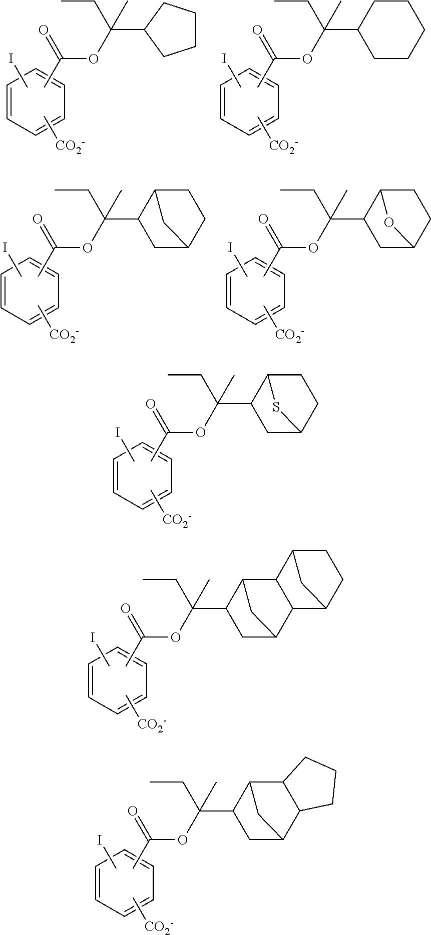

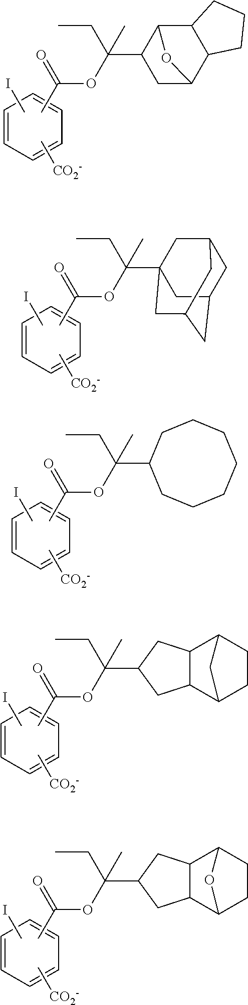

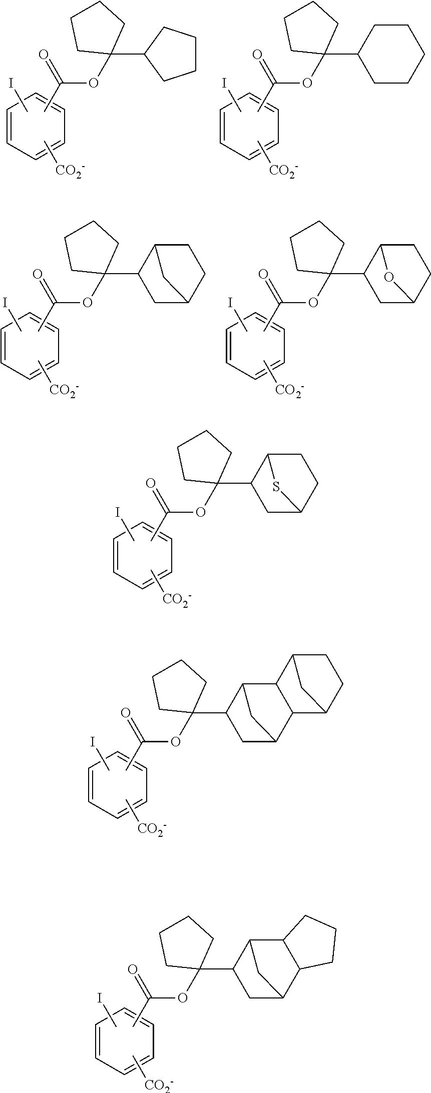

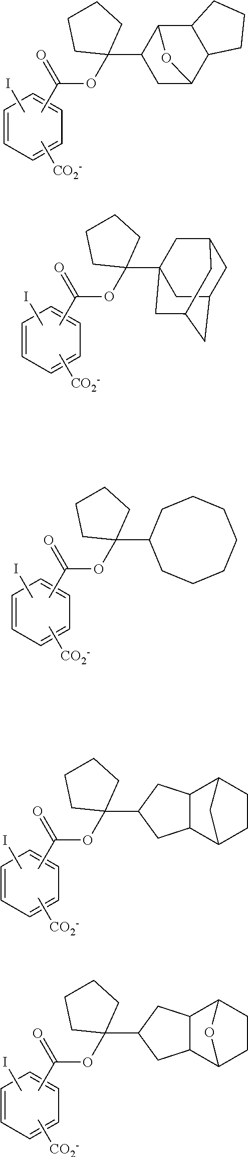

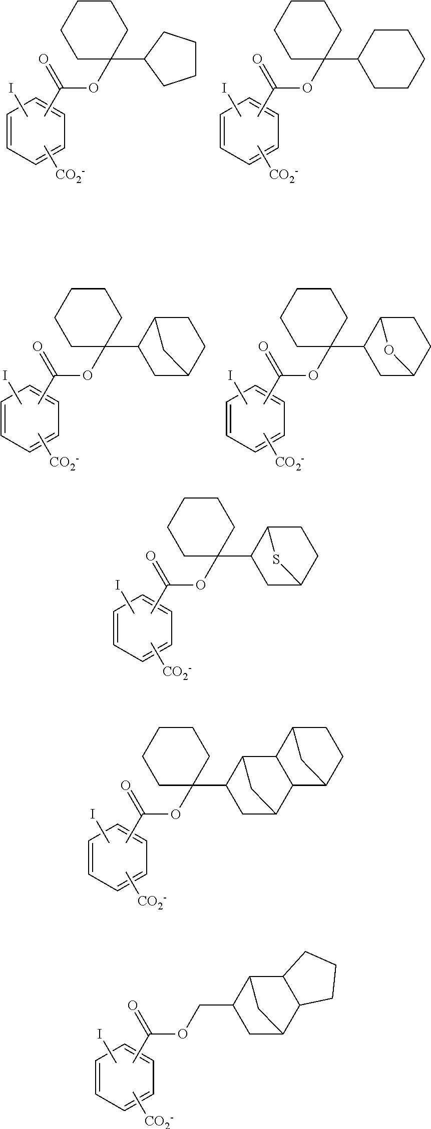

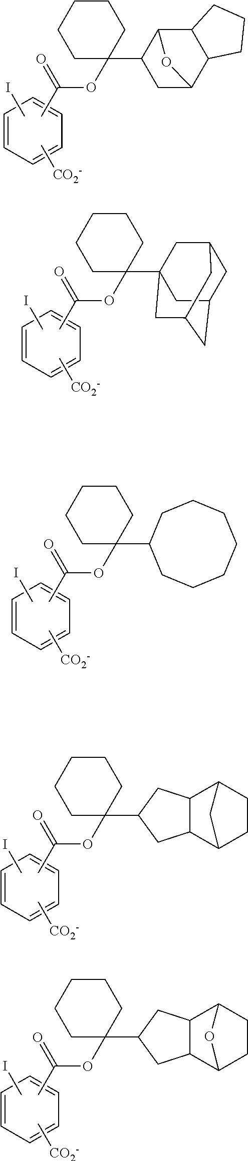

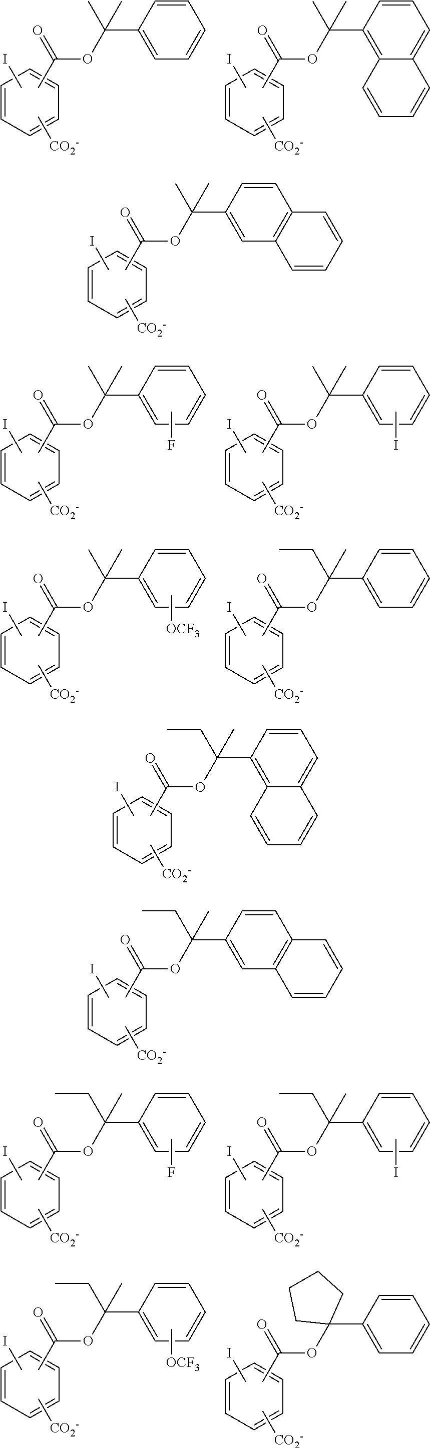

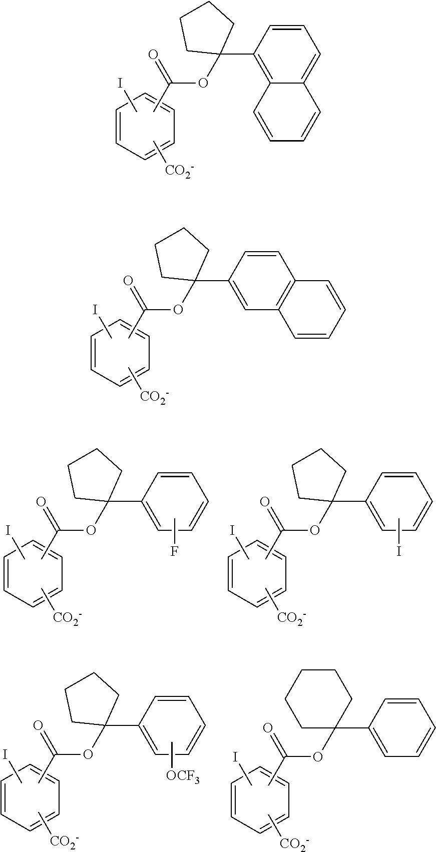

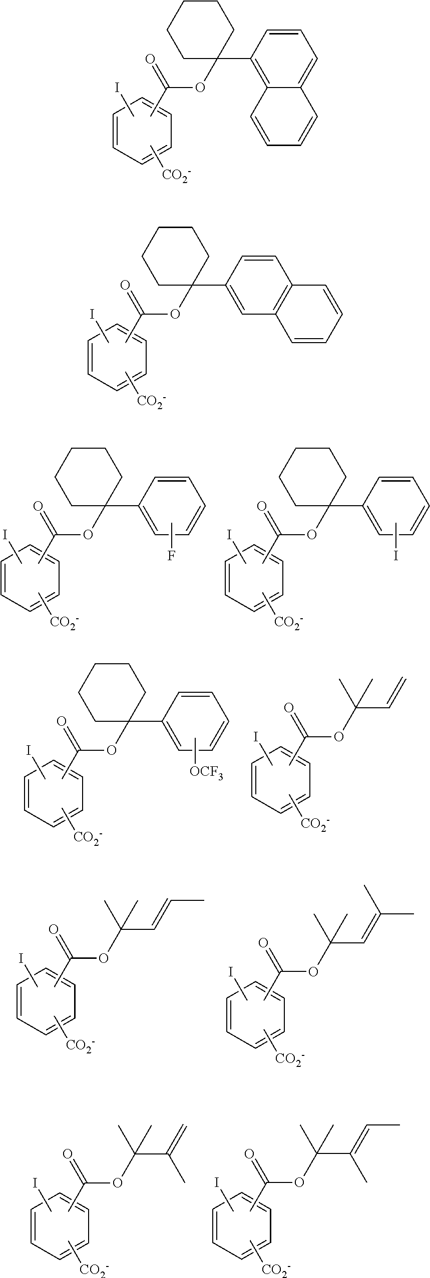

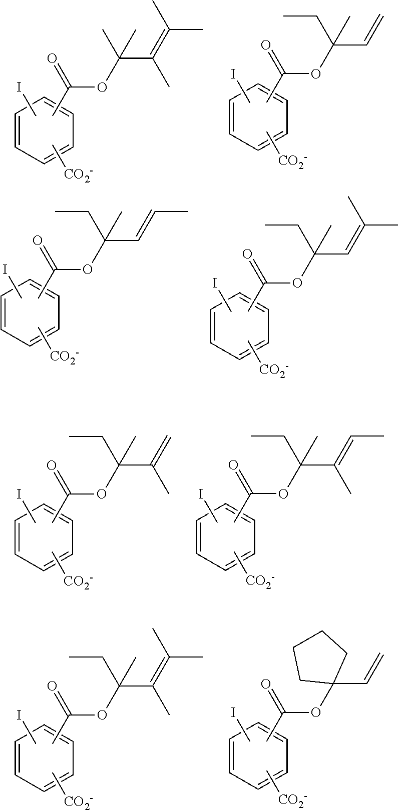

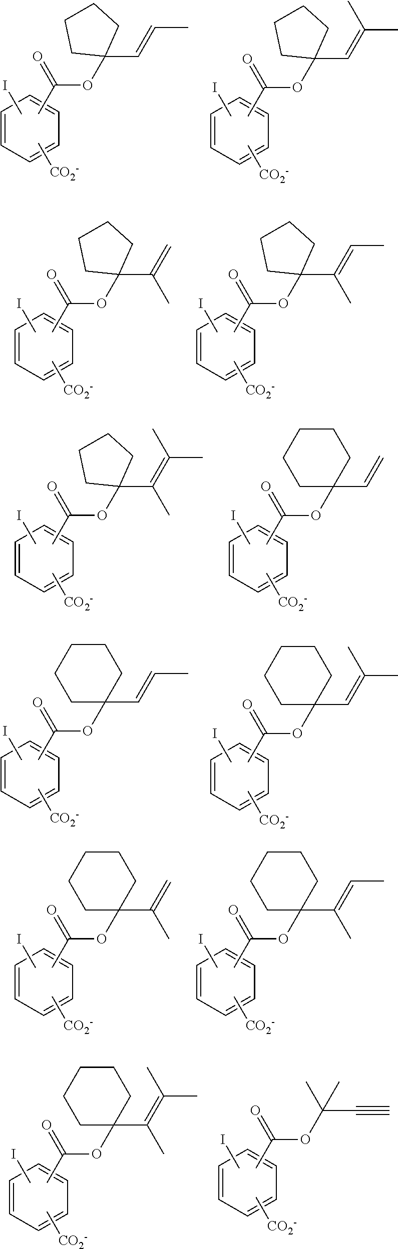

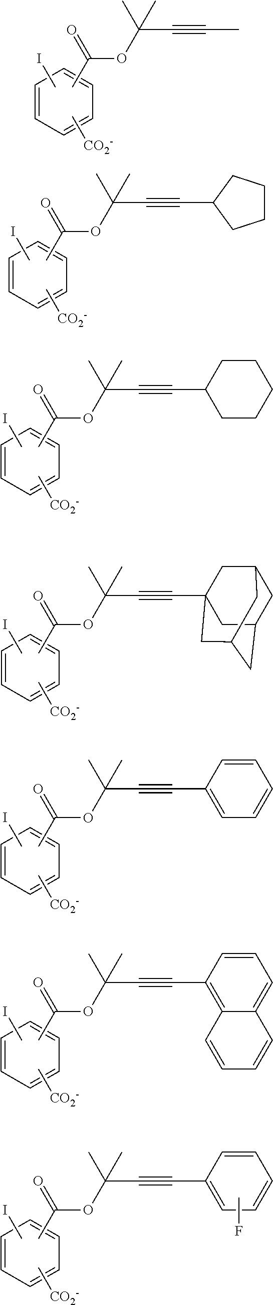

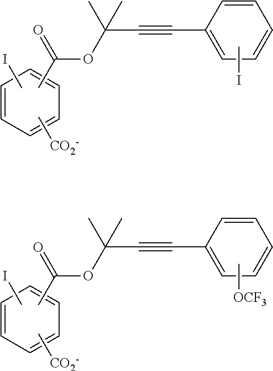

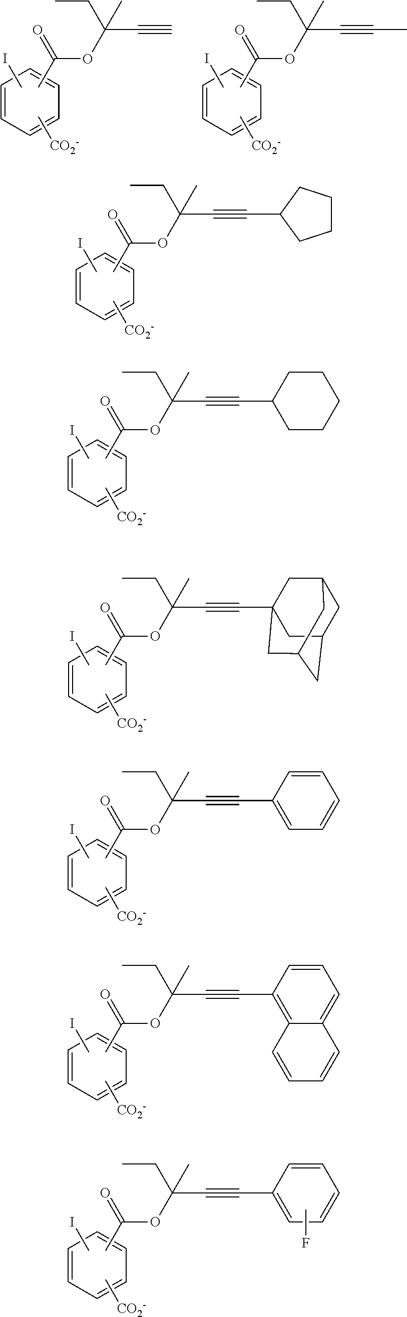

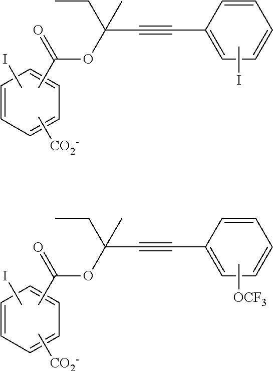

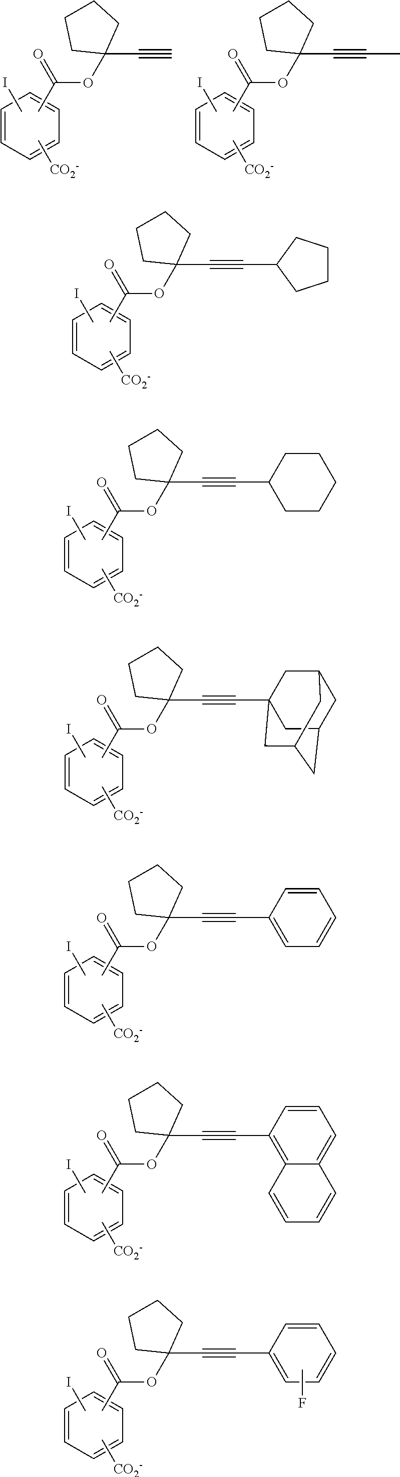

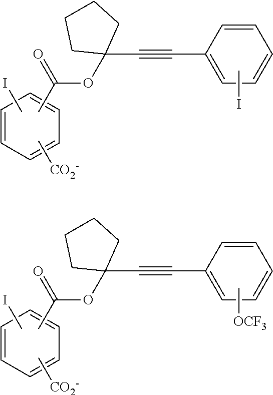

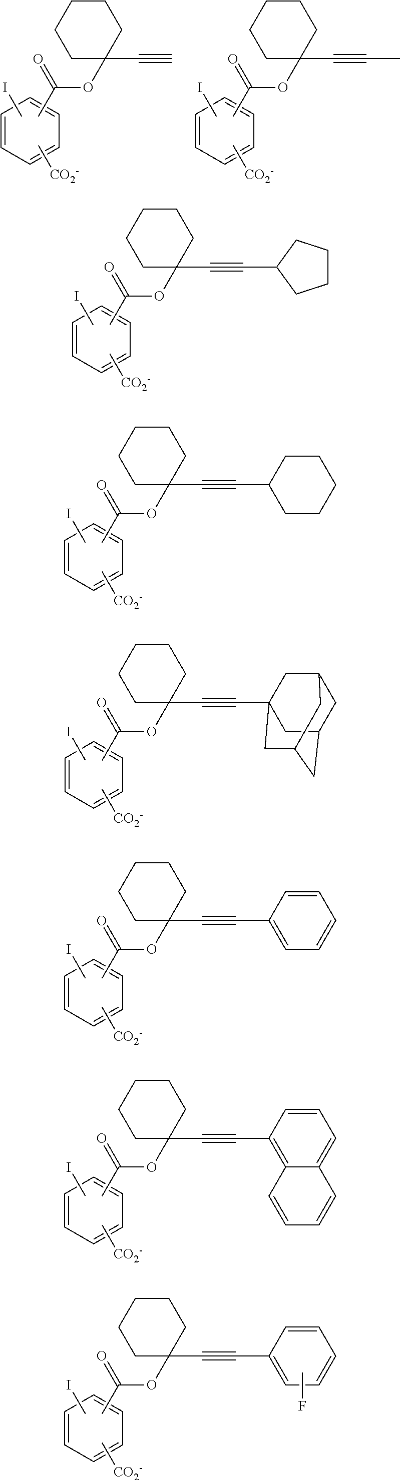

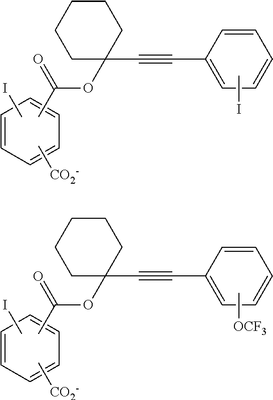

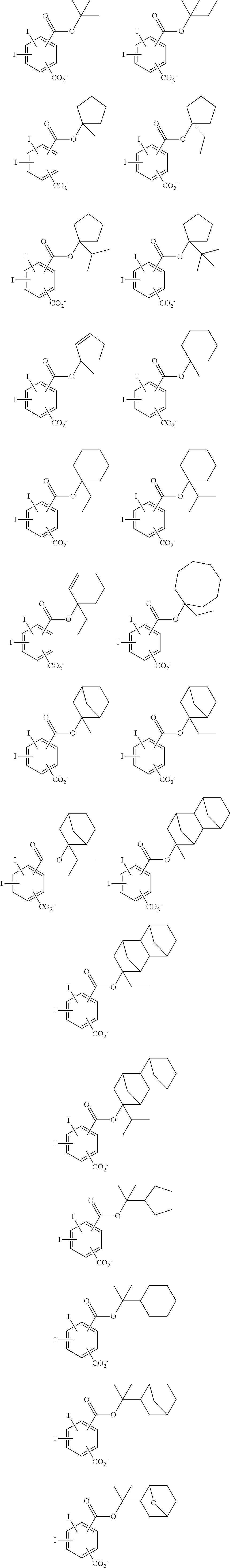

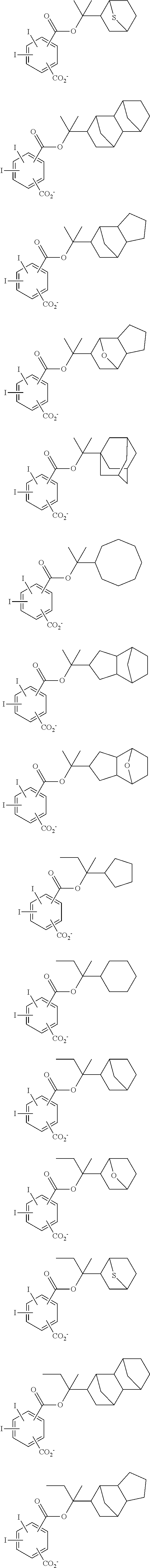

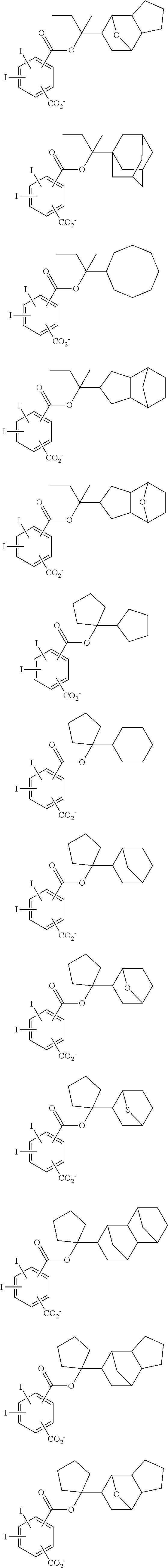

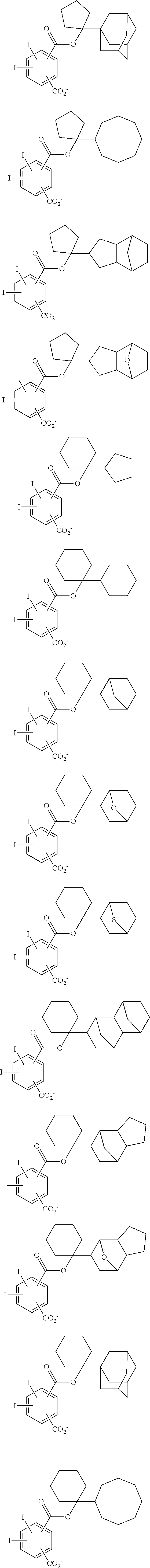

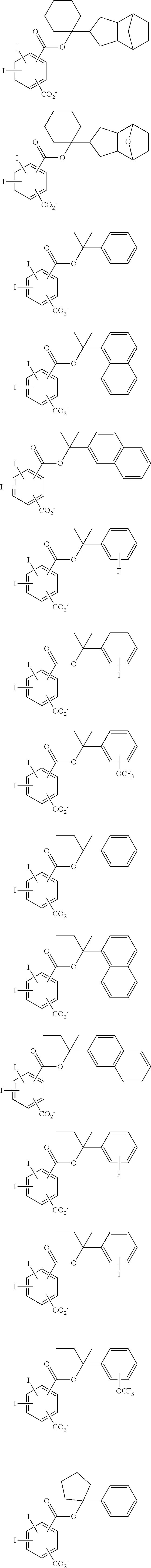

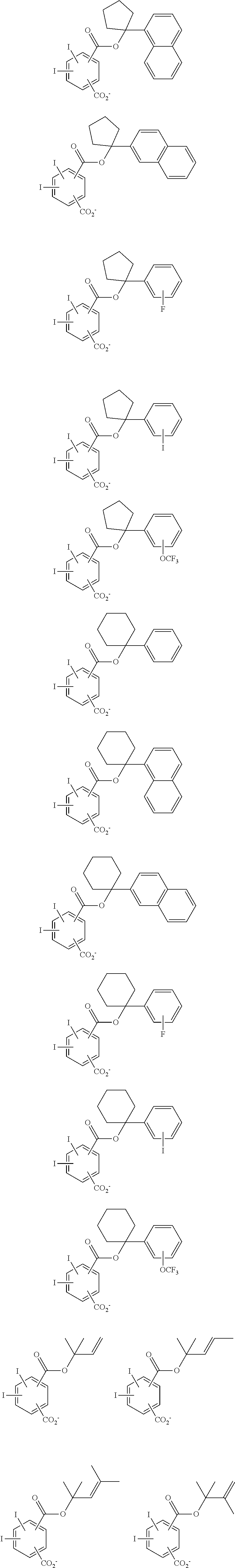

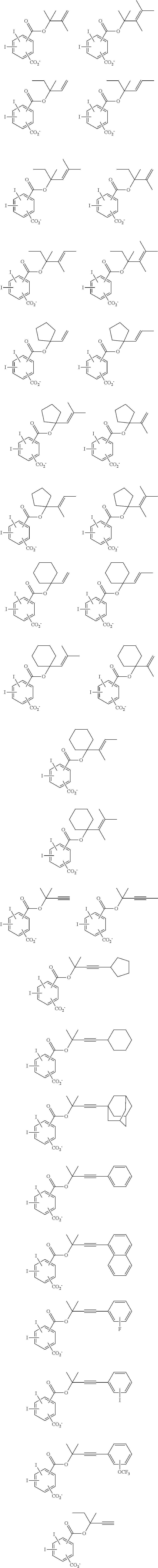

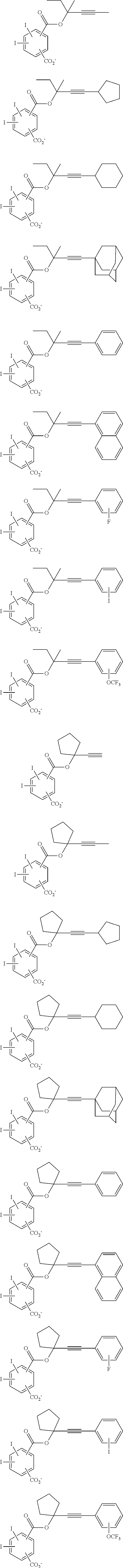

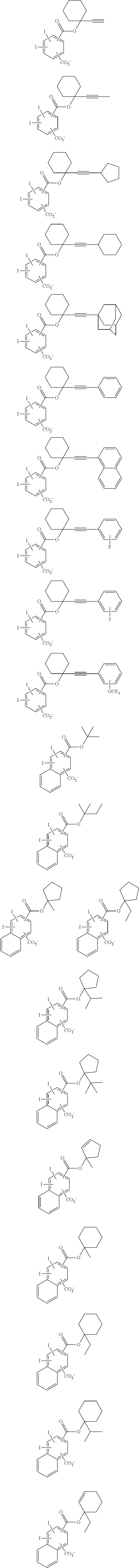

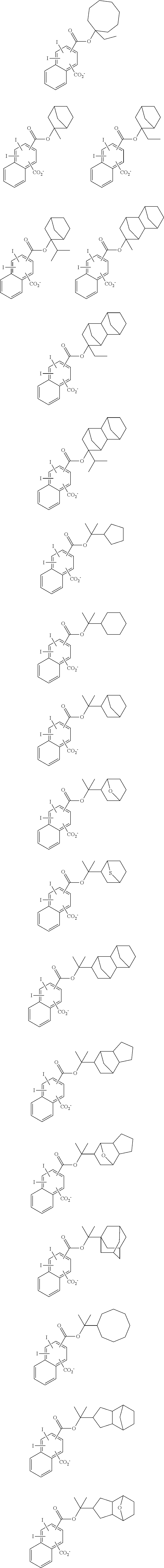

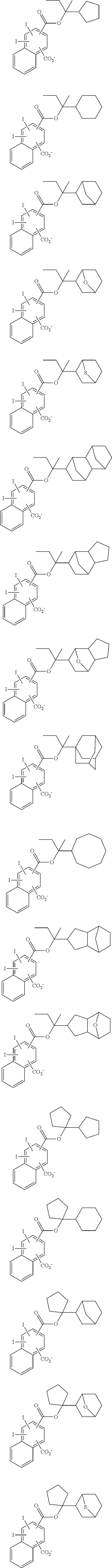

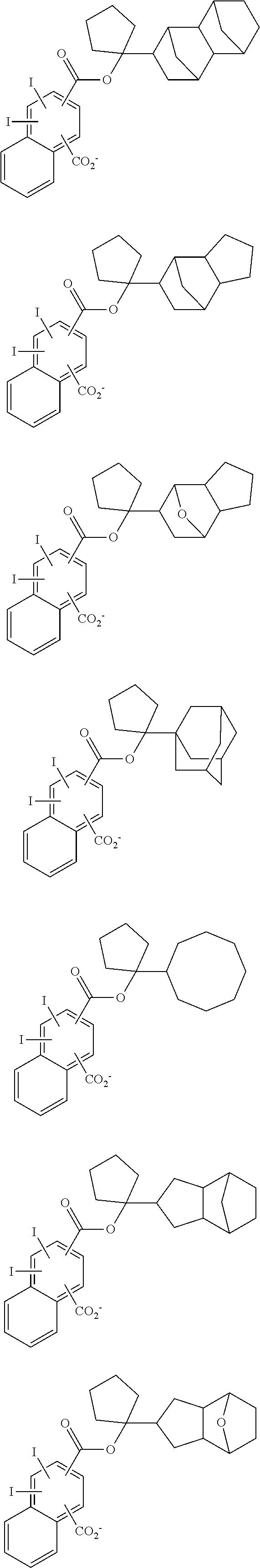

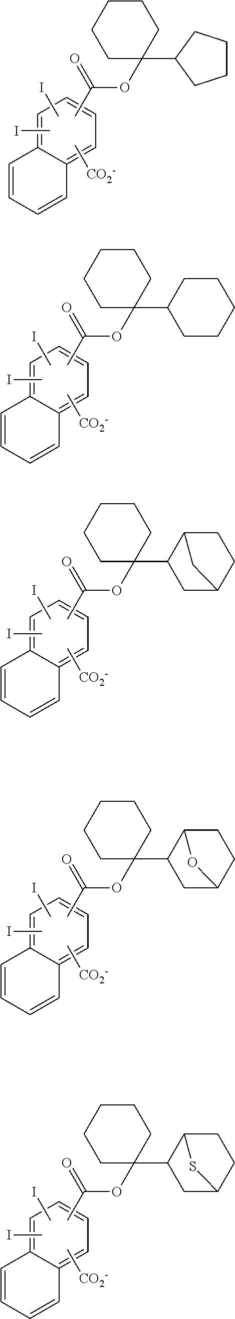

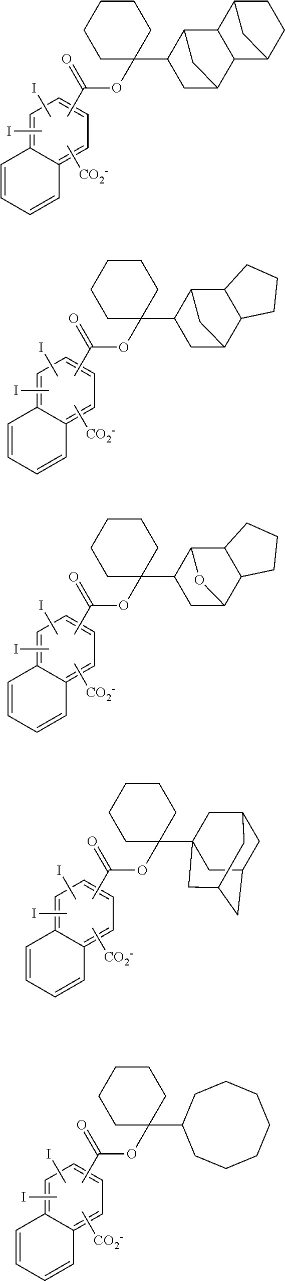

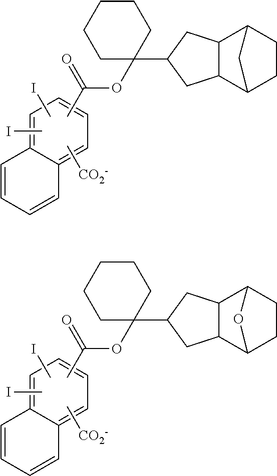

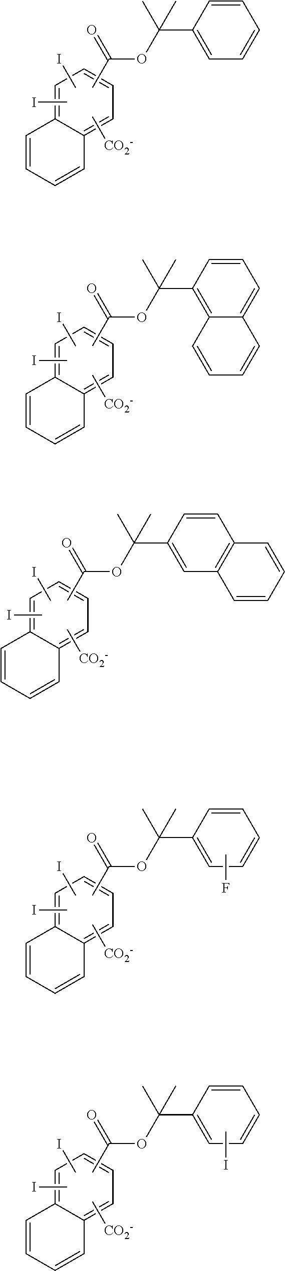

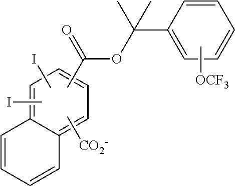

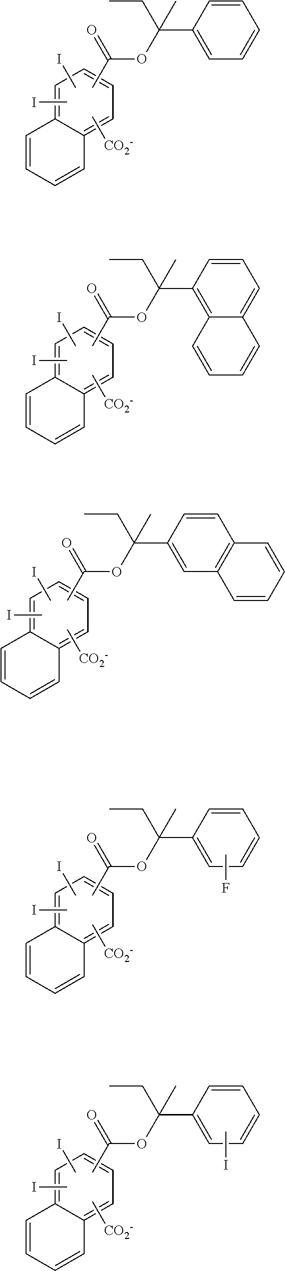

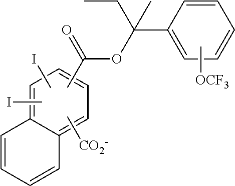

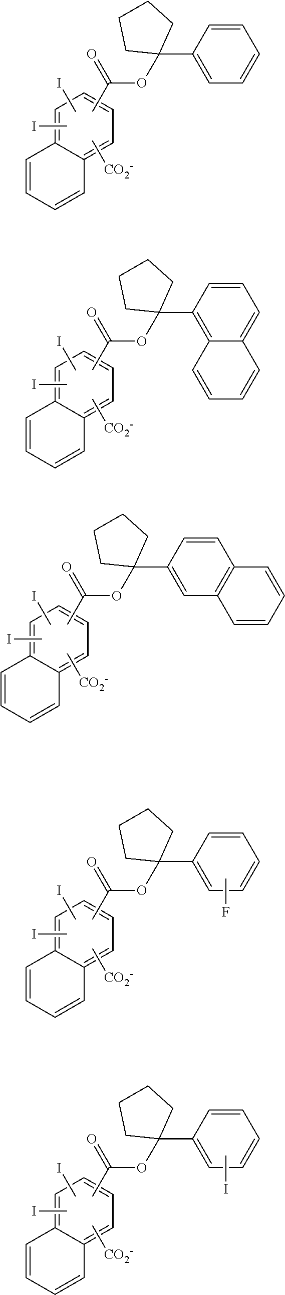

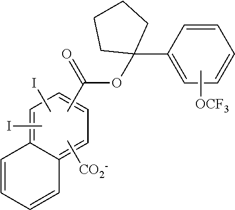

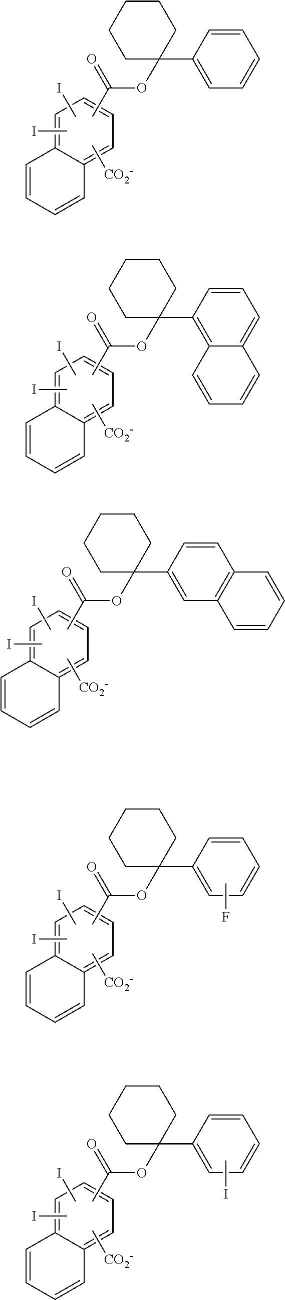

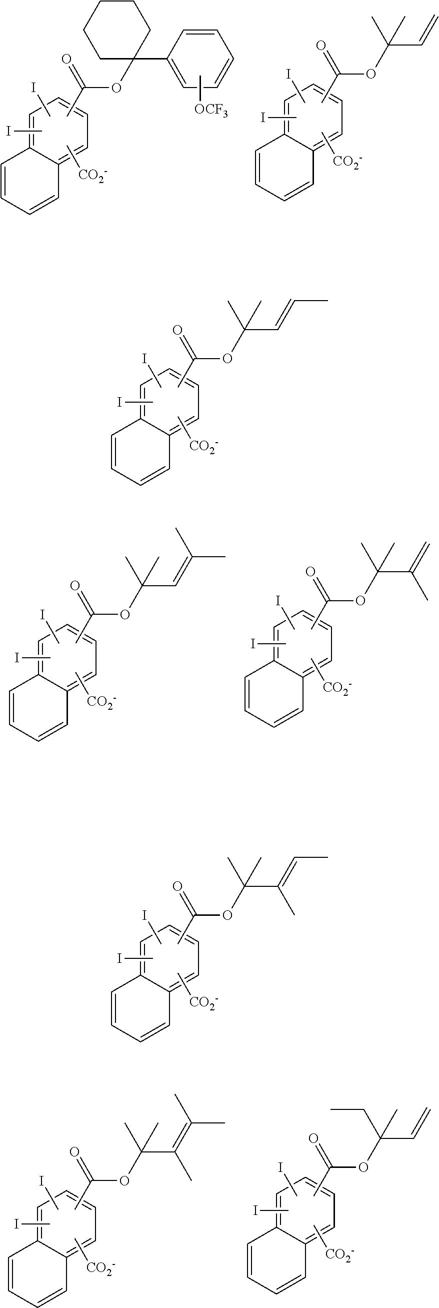

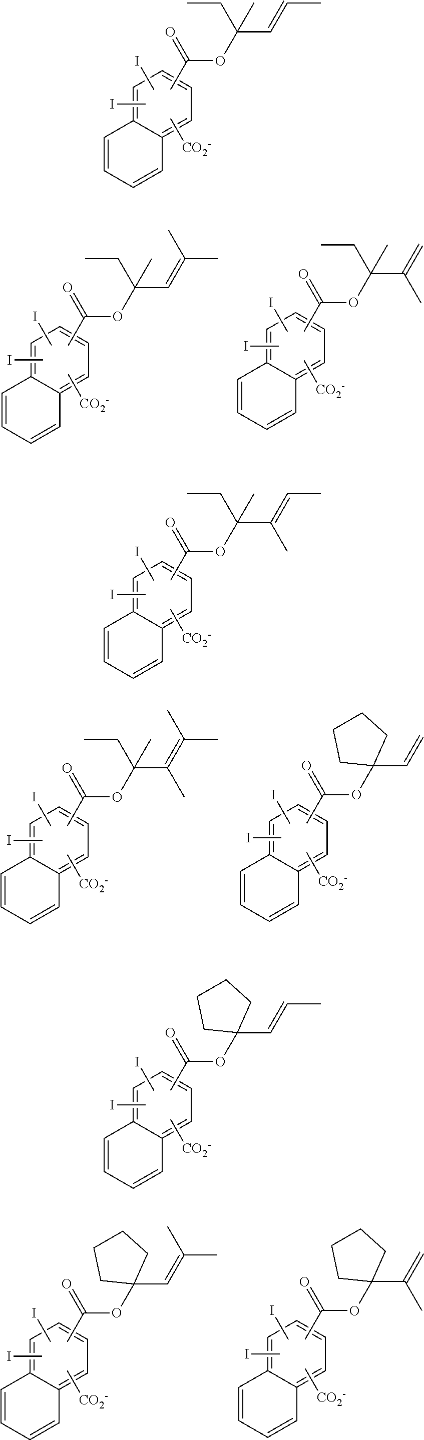

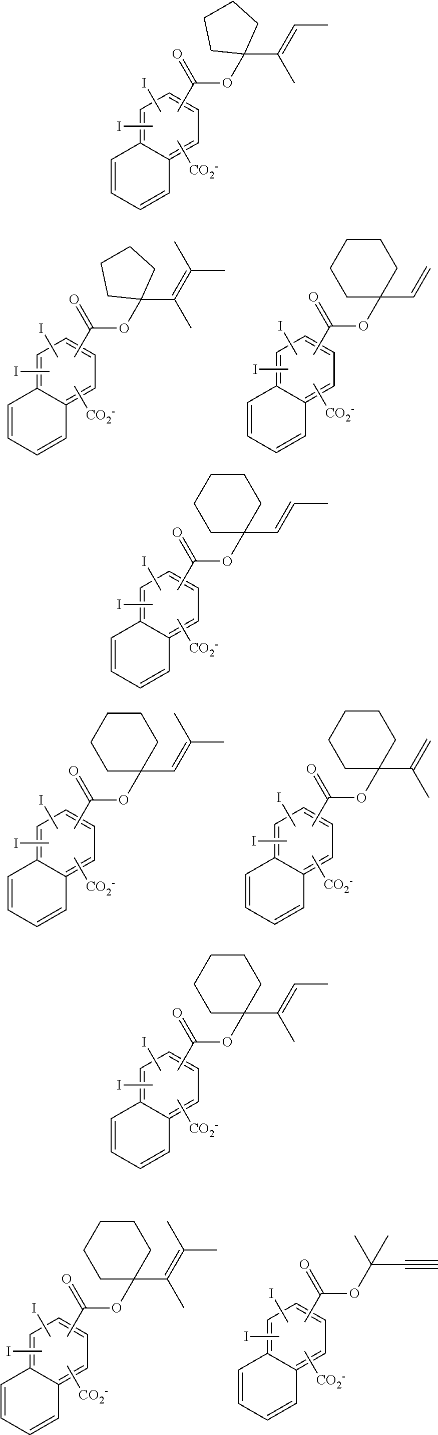

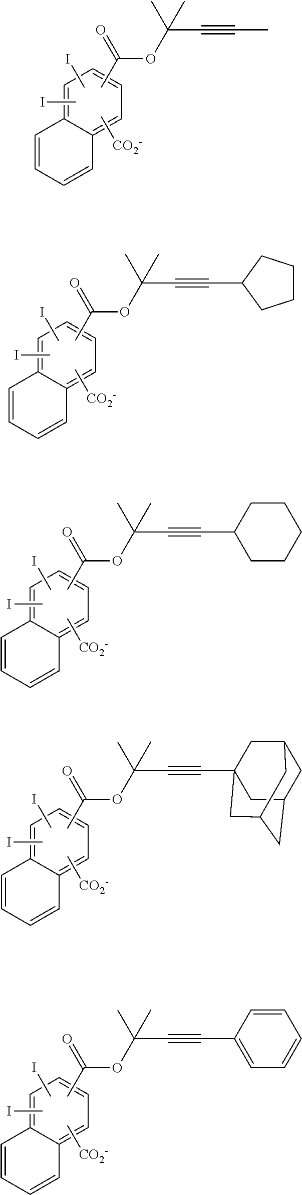

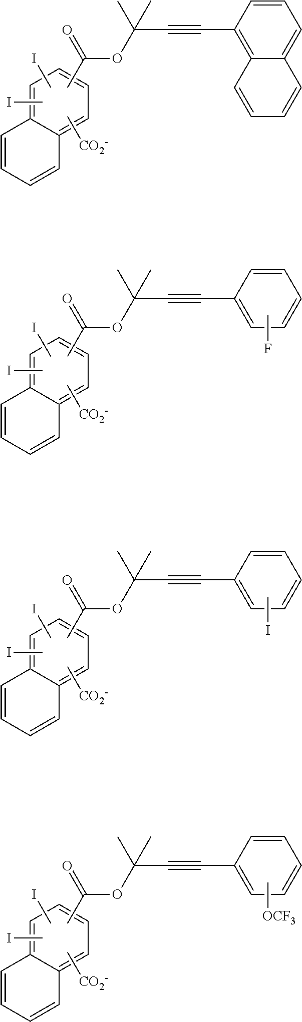

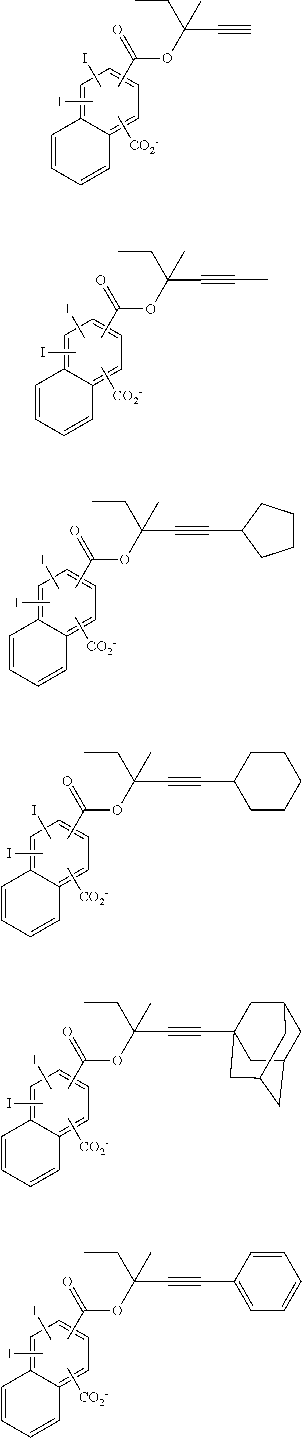

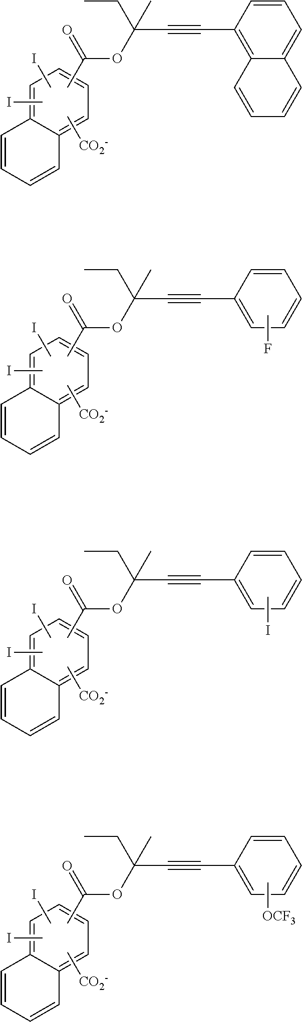

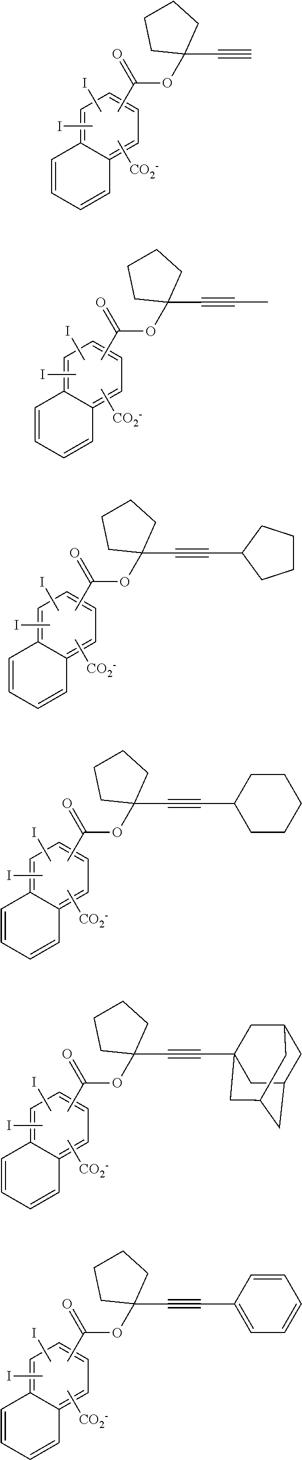

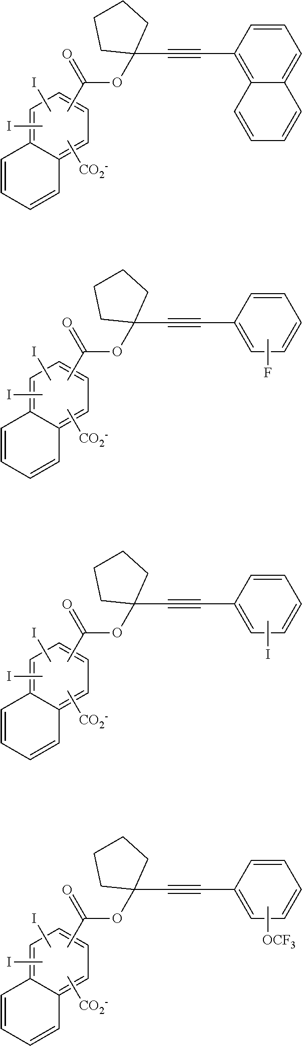

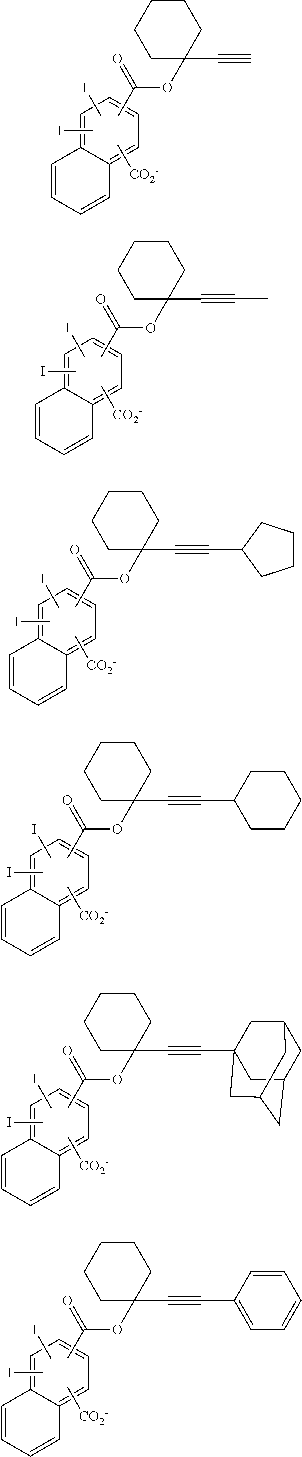

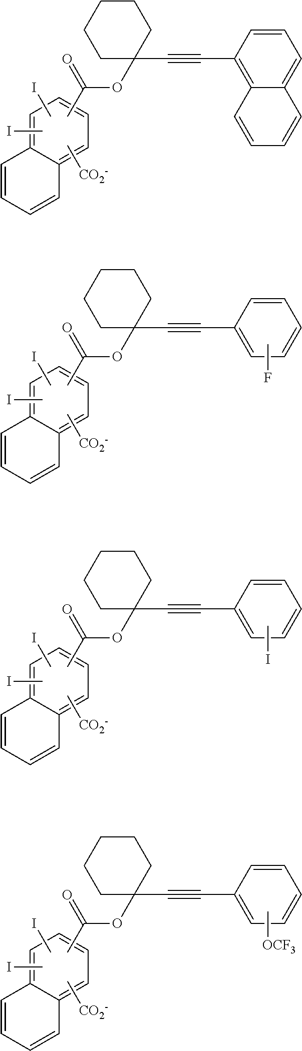

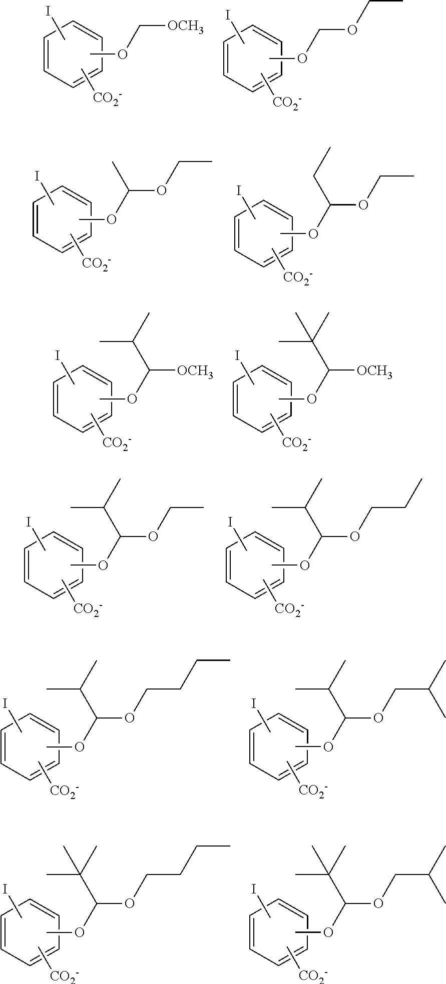

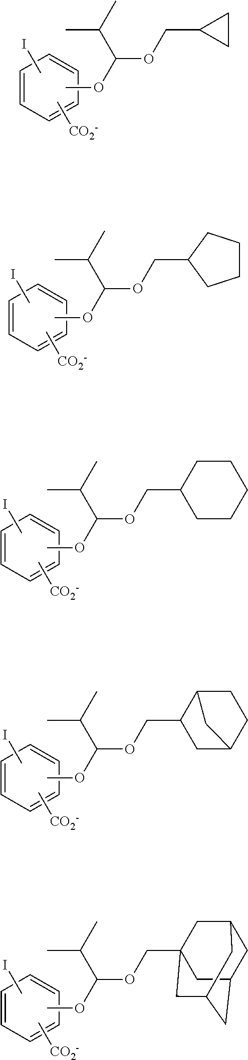

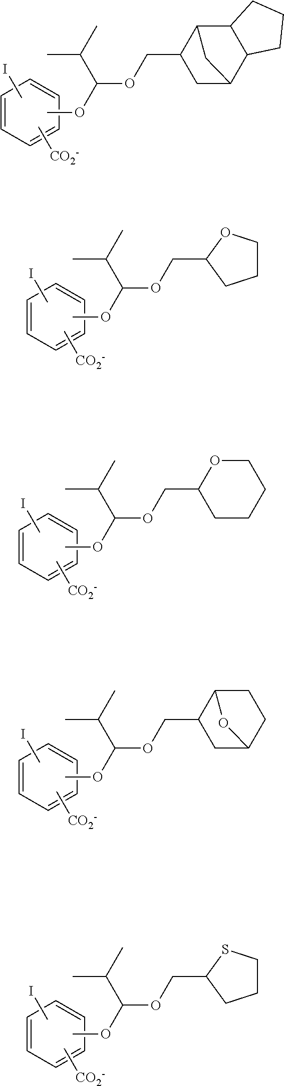

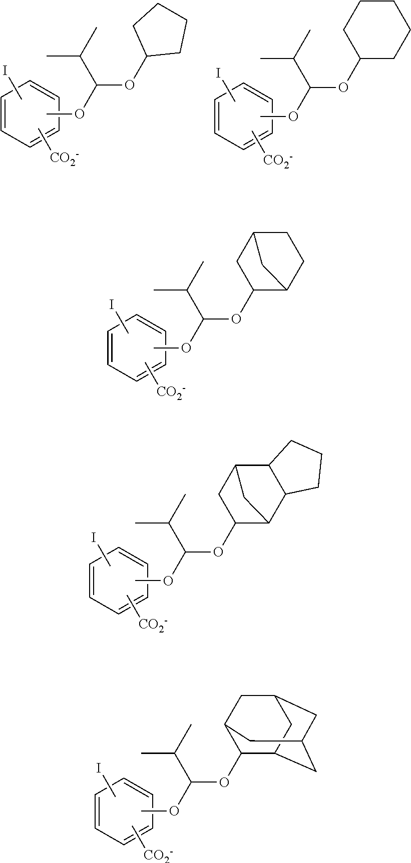

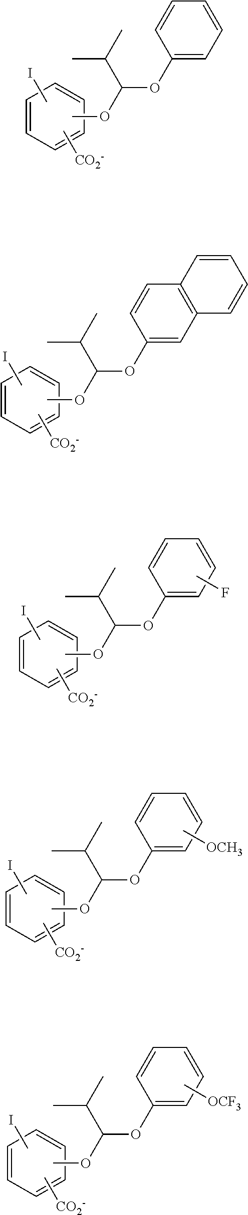

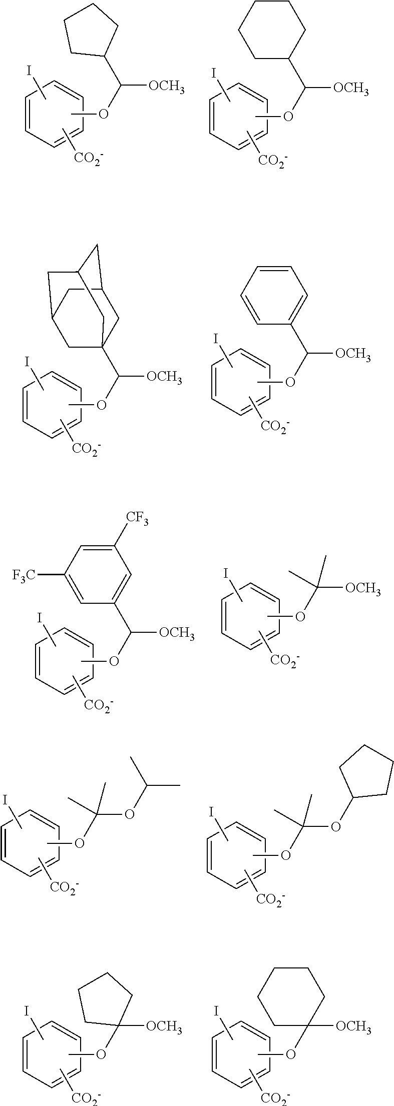

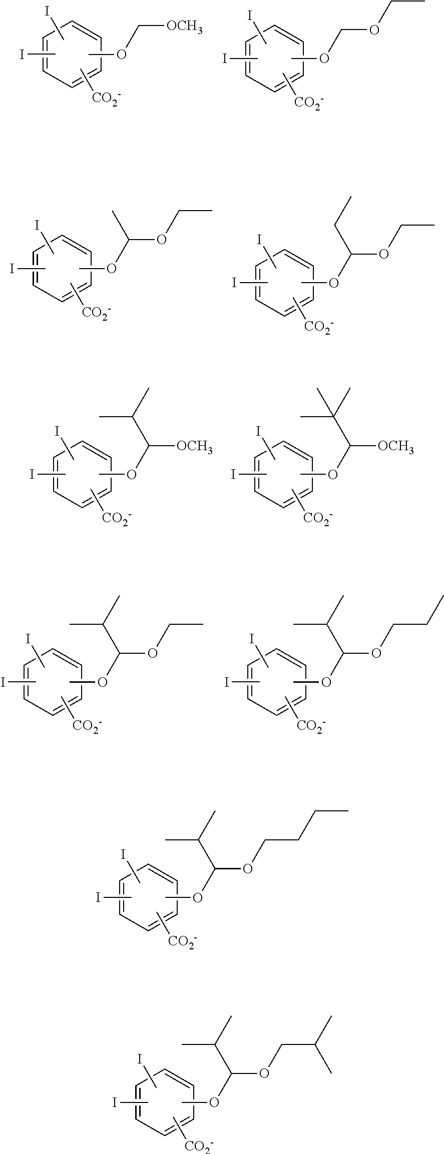

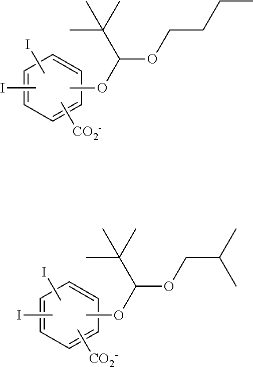

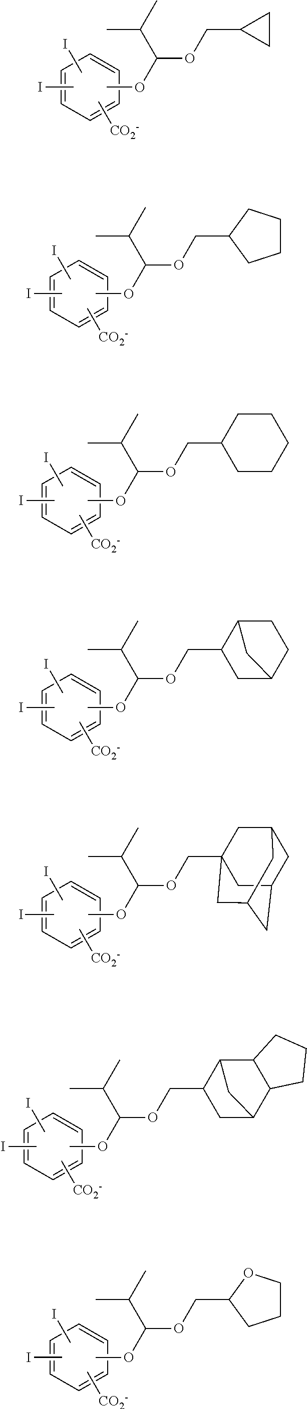

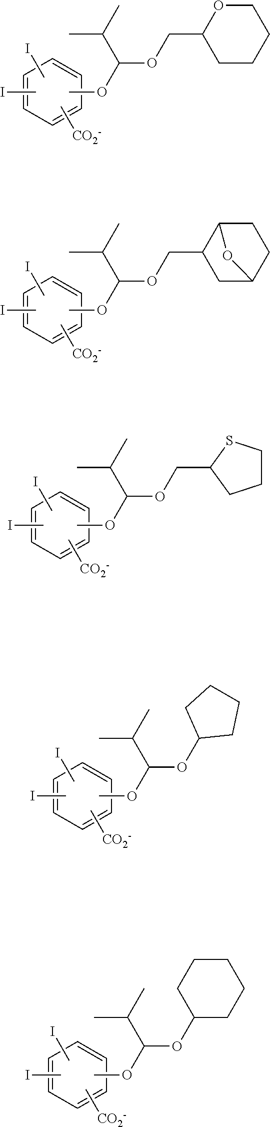

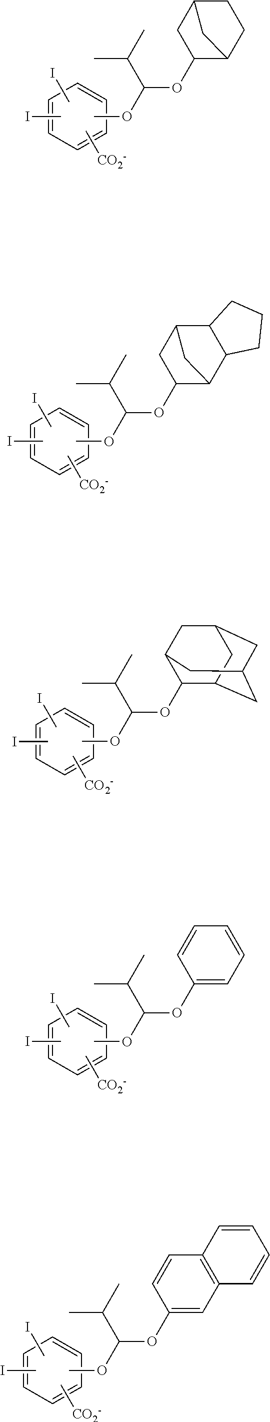

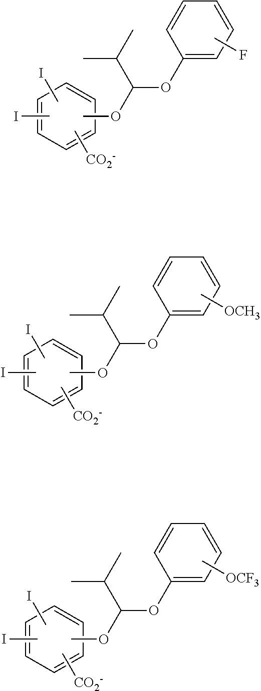

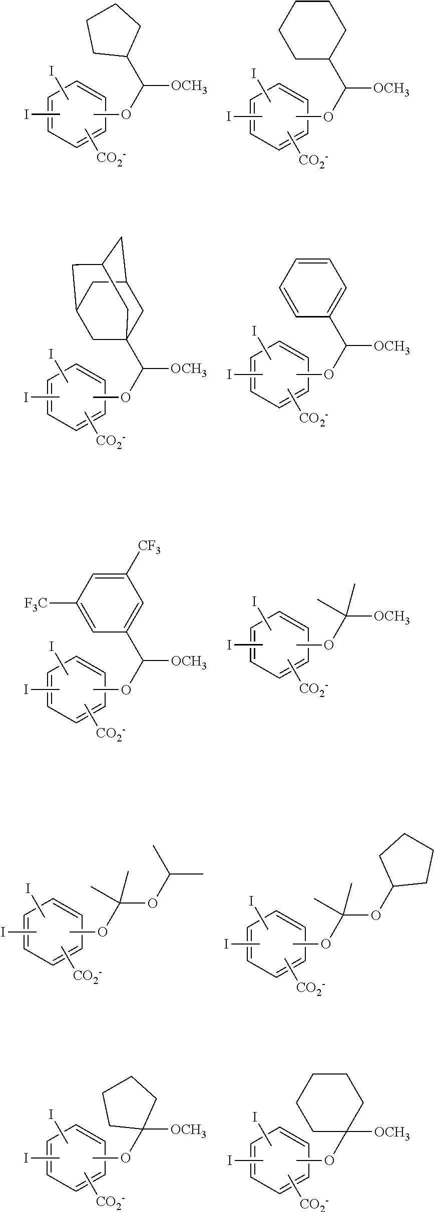

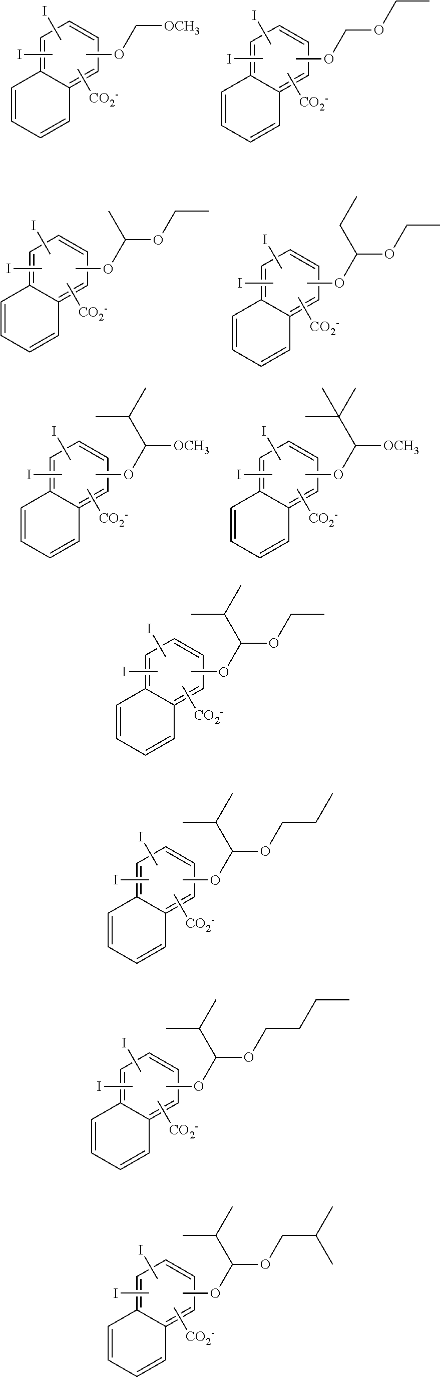

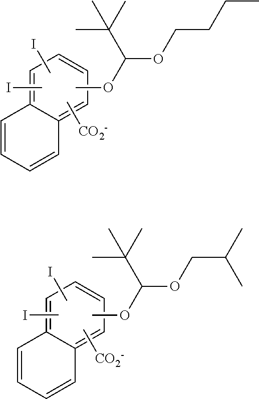

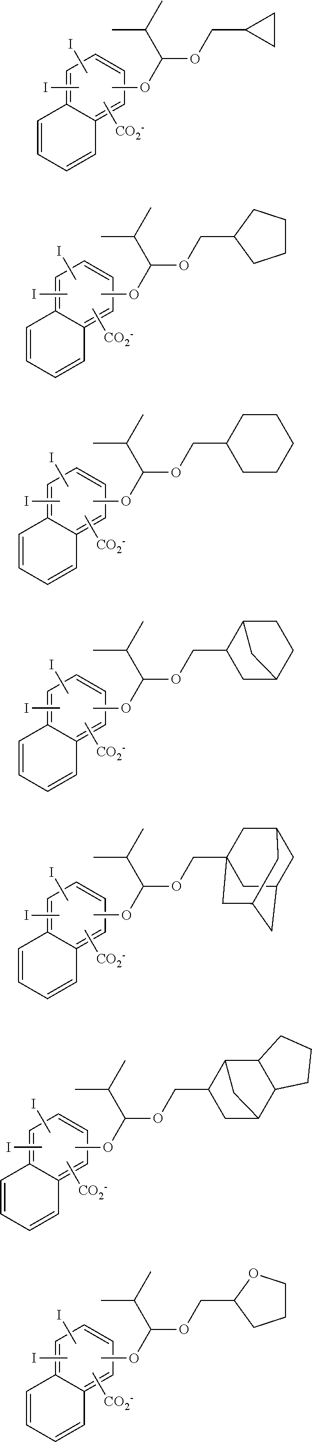

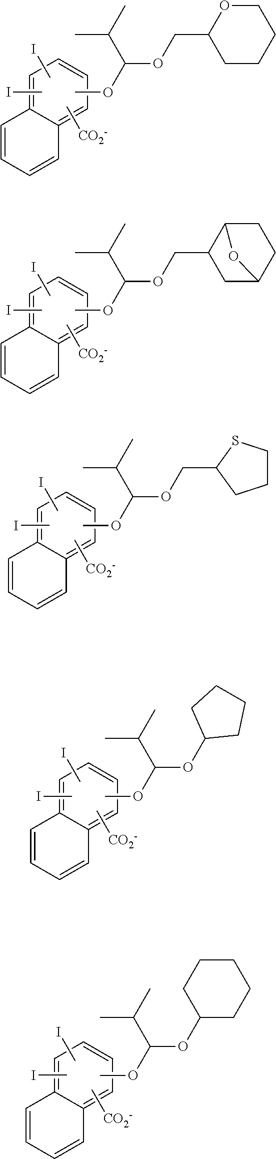

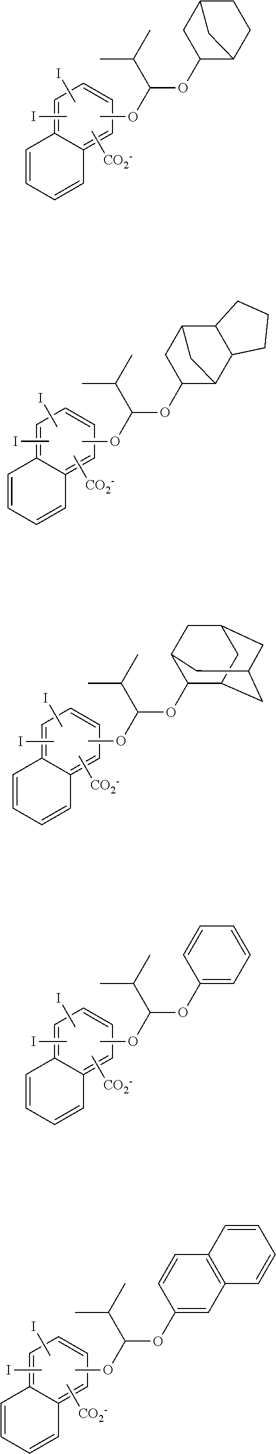

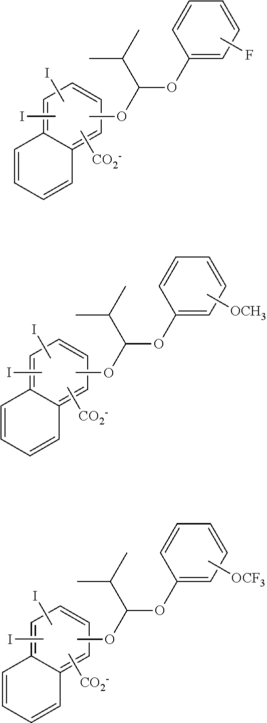

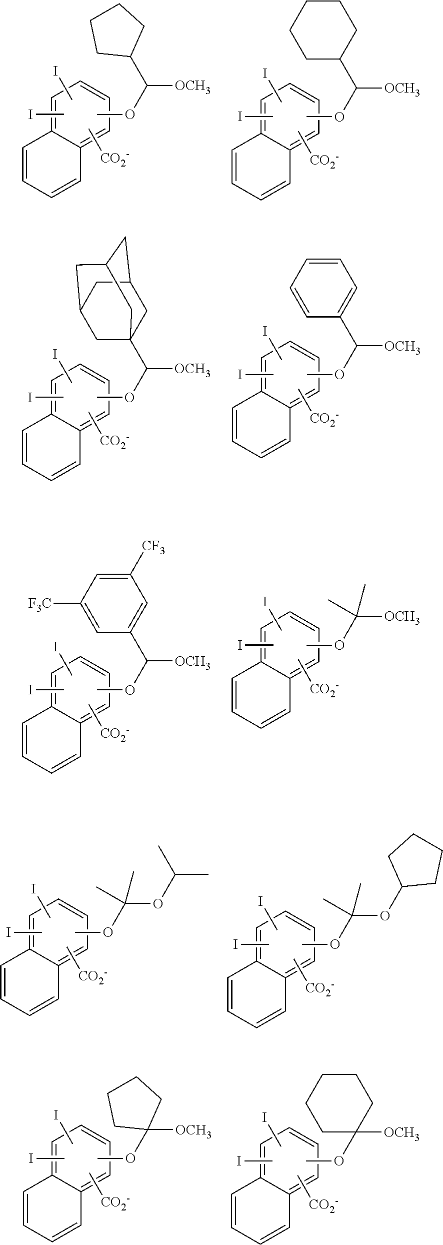

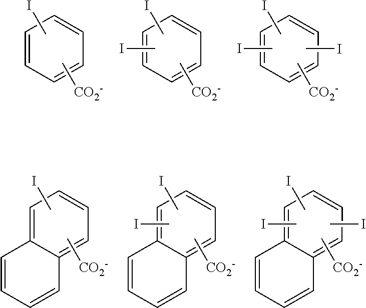

Other specific examples of the aromatic carboxylic acid anion having the formula (Z) include those described in paragraphs [0097] of WO 2023/189502, paragraphs [0231] to [0233] and [0236] of WO 2023/199907, paragraphs [0240] to [0242] of WO 2023/223865, paragraphs [0203] to [0211] of WO 2024/122425, paragraphs [0282] to [0294] of WO 2024/166822, paragraphs [0013] to [0015] of JP-A 2022-077505, paragraphs [0022] to [0024] of JP-A 2022-091720, paragraphs [0026] to [0031] of JP-A 2022-141598, paragraphs [0017] and [0018] of JP-A 2023-018671, paragraph [0157] of JP-A 2023-062898, paragraph [0147] of JP-A 2023-108593, paragraphs [0181] to [0194] of JP-A 2024-040793, paragraph [0171] of JP-A 2024-067732, paragraphs [0029] and [0030] of JP-A 2024-074267, paragraph [0022] of JP-A 2024-074268, paragraphs [0233] to [0237] of JP-A 2024-079160, paragraphs [0025] and [0026] of JP-A 2024-083302, paragraphs [0030] and [0031] of JP-A 2024-083305, paragraphs [0032] to [0034] of JP-A 2024-084141, paragraphs [0030] and [0031] of JP-A 2024-099498, paragraphs [0032] and [0033] of JP-A 2024-099503, paragraphs [0032] and [0033] of JP-A 2024-101558, paragraphs [0027] and [0028] of JP-A 2024-102839, paragraphs [0035] and [0036] of JP-A 2024-102841, paragraphs [0256] to [0261] of JP-A 2024-113453, paragraph [0266] of JP-A 2024-131484, paragraphs [0022] to [0024] of JP-A 2025-010082, paragraph [0299] of JP-A 2025-027484, paragraphs [0190] to [0198] of JP 7611887, paragraphs [0285] to [0290] of JP 7614747, and paragraphs [0201] to [0209] of JP 7621323.

Specific examples of the sulfonium salt of the present invention include any combination of the anion and the cation described above.

The sulfonium salt of the present invention can be synthesized, for example, by a similar method to a method for an onium salt described in JP-A 2020-91312, but is not limited thereto.

Structural characteristics of the sulfonium salt of the present invention include having a triarylsulfonium cation having a nitro group and an acid-unstable group having a tertiary/secondary ether structure, a tertiary/secondary ester structure, a tertiary/secondary carbonate structure, or an acetal structure on the same aromatic ring and an aromatic carboxylic acid anion which may have a substituent. The substituent of the aromatic carboxylic acid anion is preferably an iodine atom, and the aromatic carboxylic acid anion containing an iodine atom generates secondary electrons from the iodine atom during exposure because of having very large absorption of EUV by the iodine atom particularly in EUV lithography having a wavelength of 13.5 nm. In addition, the iodine atom has an appropriate dissolution inhibiting ability, and suppresses pattern collapse of an unexposed portion particularly with respect to an alkaline developer. The nitro group bonded to an aromatic ring of the sulfonium cation is known as a strong electron-withdrawing group. From this, it is considered that an energy level of LUMO in the frontier orbital theory decreases. Therefore, secondary electrons generated from an iodine atom in the anion are easily received, decomposition of the cation is promoted, and an acid is efficiently generated. In addition, the nitro group has a resonance structure in which a positive charge is provided on a nitrogen atom thereof and a negative charge is provided on one oxygen atom thereof, and therefore can also be expected to function as an acid diffusion inhibiting group by interacting with a proton of a generated acid at the negative charge on the oxygen atom. By being charged as described above, affinity with an alkaline developer is high, and a residue after cationic decomposition is efficiently removed from the alkaline developer. Therefore, a risk of development defects in an exposed portion is also reduced.

The acid-unstable group having a tertiary/secondary ether structure, a tertiary/secondary ester structure, a tertiary/secondary carbonate structure, or an acetal structure undergoes a deprotection reaction with an acid generated by exposure to generate a corresponding phenol or carboxylic acid. By change of the structure from a fat-soluble structure to a hydrophilic structure, contrast between an exposed portion and an unexposed portion is improved. In addition, a nitro group is bonded to a carbon atom adjacent to a carbon atom to which the phenolic hydroxy group or the carboxy group generated by the deprotection reaction is bonded, whereby acidity of the phenol or the carboxylic acid is improved due to an electron withdrawing property of the nitro group. When the exposed portion in which the deprotection reaction has progressed is developed with an alkaline developer, the phenol having an improved acidity has improved solubility in the alkaline developer, whereby development residues can be suppressed. In addition, the triarylsulfonium cation preferably has a fluorine atom or a substituent containing a fluorine atom on another aromatic ring therein. The fluorine atom or the substituent containing a fluorine atom has a strong electron withdrawing property, and it is considered that an energy level of LUMO in the frontier orbital theory of the triarylsulfonium cation decreases. Therefore, secondary electrons generated from iodine atoms in the cation and the anion are easily received, decomposition of the cation is promoted, and an acid is efficiently generated. In addition, in a case where the anion also has an acid-unstable group, a deprotection reaction of the acid-unstable group by a generated acid proceeds, and improvement in contrast and high sensitivity due to further change in solubility are expected. In an exposed portion of a positive resist, the higher the affinity for an alkaline developer, the lower a risk of development defects. Furthermore, the sulfonium salt of the present invention has an aromatic carboxylic acid anion, therefore has relatively strong basicity, and effectively traps a strong acid generated from a photoacid generator. Due to a synergistic effect of these, when the sulfonium salt of the present invention is used, dissolution contrast is high, lithographic performance such as LWR of a line pattern or CDU of a hole pattern is excellent, and a pattern resistant to pattern collapse can be formed. Therefore, the sulfonium salt is suitable as a material for a positive resist composition.

The sulfonium salt of the present invention can be suitably used as a quencher. Note that, in the present invention, the quencher is a material for forming a desired pattern by trapping a strong acid generated from a photoacid generator to prevent diffusion thereof to an unexposed portion. The photoacid generator is a compound that generates a strong acid by irradiation with a high energy ray, and the strong acid is a compound having an acidity sufficient to cause a deprotection reaction of an acid-unstable group. When the acid-unstable group is a tertiary ester or a tertiary ether, the aromatic carboxylic acid has no acidity enough to cause a deprotection reaction. Therefore, as described below, it is effective to separately add a photoacid generator that generates a sulfonic acid, an imidic acid, or a methidic acid whose α-position is fluorinated, which is a strong acid, in order to cause a deprotection reaction of the acid-unstable group. Note that the photoacid generator that generates a sulfonic acid, an imidic acid, or a methidic acid whose α-position is fluorinated may be an additive type, or may be a polymer-bound type in which the photoacid generator is bonded to a base polymer.

When light irradiation is performed in a state where a sulfonium salt that generates the aromatic carboxylic acid and a photoacid generator that generates perfluoroalkylsulfonic acid as a superstrong acid are mixed, the aromatic carboxylic acid and the perfluoroalkylsulfonic acid are generated. Since the whole amount of the photoacid generator is not decomposed, there is the photoacid generator that is not decomposed in the vicinity. Here, when the sulfonium salt that generates the aromatic carboxylic acid and a perfluoroalkylsulfonic acid coexist, first, the perfluoroalkylsulfonic acid causes ion exchange with the sulfonium salt that generates the aromatic carboxylic acid, and a perfluoroalkylsulfonic acid sulfonium salt is generated to release the aromatic carboxylic acid. This is because the perfluoroalkyl sulfonate having high strength as an acid is more stable. On the other hand, even when the perfluoroalkylsulfonic acid sulfonium salt and the aromatic carboxylic acid are present, ion exchange does not occur. The ion exchange occurs not only in the perfluoroalkylsulfonic acid and similar ion exchange occurs also in arenesulfonic acid, alkylsulfonic acid, imidic acid, methidic acid, or the like having higher acid strength than the aromatic carboxylic acid generated from the sulfonium salt of the present invention.

### [Chemically Amplified Resist Composition]

### Quencher (A)

The chemically amplified resist composition of the present invention contains a quencher formed of a sulfonium salt having the formula (1) as an essential component.

The content of the quencher (A) in the chemically amplified resist composition of the present invention is preferably 0.1 to 40 parts by weight, and more preferably 0.5 to 30 parts by weight per 80 parts by weight of a base polymer described below. If the content of the quencher (A) is in the above range, sensitivity and resolution are favorable, and there is no possibility that a problem of foreign matters occurs after development of a resist film or during peeling of the resist film, which is preferable. The quencher (A) may be used singly or in combination of two or more types thereof.

### Base Polymer (B)

The chemically amplified resist composition of the present invention may contain a base polymer as a component (B). The base polymer (B) preferably contains a repeat unit having an acid-unstable group. The repeat unit containing an acid-unstable group may be used singly or in combination of two or more types thereof.

Specific examples of the repeat unit containing an acid-unstable group include a repeat unit having the following formula (a1) (hereinafter, also referred to as a repeat unit a1) and a repeat unit having the following formula (a2) (hereinafter, also referred to as a repeat unit a2).

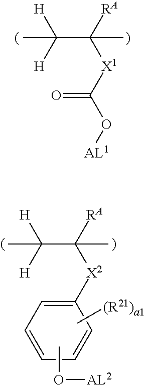

In the formulas (a1) and (a2), R^{A}s are each independently a hydrogen atom, a fluorine atom, a methyl group, or a trifluoromethyl group.

In the formula (a1), X¹ is a single bond, a phenylene group, a naphthylene group, *—C(═O)—O—X¹¹—, or *—C(═O)—N(H)—X¹¹—, and the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom. X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring. * represents a bond with a carbon atom of a main chain.

In the formula (a2), X² is a single bond, *—C(═O)—O—, or *—C(═O)—N(H)—X¹¹—. * represents a bond with a carbon atom of a main chain. R²¹ is a halogen atom, a cyano group, a hydroxy group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. a1 is 0, 1, 2, 3, or 4, and preferably 0 or 1. When a1 is 2, 3, or 4, R²¹s may be the same as or different from each other.

In the formulas (a1) and (a2), AL¹ and AL² are each independently an acid-unstable group. Specific examples of the acid-unstable group include those described in JP-A 2013-80033 and JP-A 2013-83821.

Typically, specific examples of the acid-unstable group include those having the following formulas (AL-5) to (AL-7):

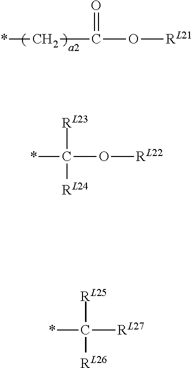

wherein the dashed line represents a bond.

In the formulas (AL-5) and (AL-6), R^{L21} and R^{L22} are each independently a C₁-C₄₀ hydrocarbyl group, and may contain a heteroatom such as an oxygen atom, a sulfur atom, a nitrogen atom, or a fluorine atom. The hydrocarbyl group may be saturated or unsaturated, and may be linear, branched, or cyclic. The hydrocarbyl group preferably has 1 to 20 carbon atoms.

In the formula (AL-5), a2 is an integer of 0 to 10, and preferably 1, 2, 3, 4 or 5.

In the formula (AL-6), R^{L23} and R^{L24} are each independently a hydrogen atom or a C₁-C₂₀ hydrocarbyl group, and may contain a heteroatom such as an oxygen atom, a sulfur atom, a nitrogen atom, or a fluorine atom. The hydrocarbyl group may be saturated or unsaturated, and may be linear, branched, or cyclic. Any two of R^{L22}, R^{L23}, and R^{L24} may be bonded to each other to form a C₃-C₂₀ ring together with a carbon atom or a carbon atom and an oxygen atom to which they are bonded. The ring is preferably a C₄-C₁₆ ring, and particularly preferably an alicyclic ring.

In the formula (AL-7), R^{L25}, R^{L26}, and R^{L27} are each independently a C₁-C₂₀ hydrocarbyl group, and may contain a heteroatom such as an oxygen atom, a sulfur atom, a nitrogen atom, or a fluorine atom. The hydrocarbyl group may be saturated or unsaturated, and may be linear, branched, or cyclic. Any two of R^{L25}, R^{L26}, and R^{L27} may be bonded to each other to form a C₃-C₂₀ ring together with a carbon atom to which they are bonded. The ring is preferably a C₄-C₁₆ ring, and particularly preferably an alicyclic ring.

Other specific examples of the acid-unstable group include those described in paragraphs [0064] to [0068] of JP-A 2023-123222 and those described in paragraphs [0013] and [0014] of JP 7492842. In these, generation of a conjugated olefin or an acrylic acid ester derivative after an acid elimination reaction is used as a driving force for progress of the reaction.

Specific examples of the repeat unit a1 include, but are not limited to, those listed below. Note that, in the following formulas, R^{A} and AL¹ are as defined above.

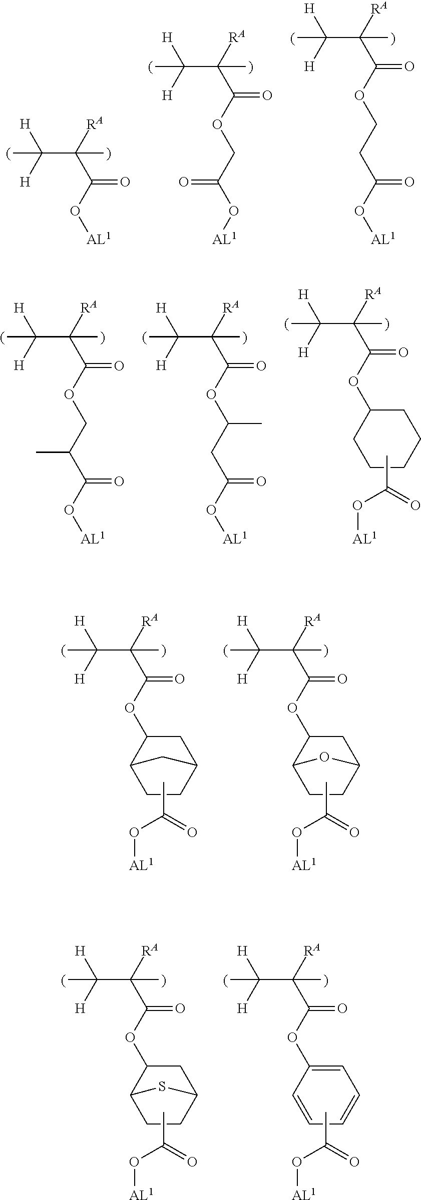

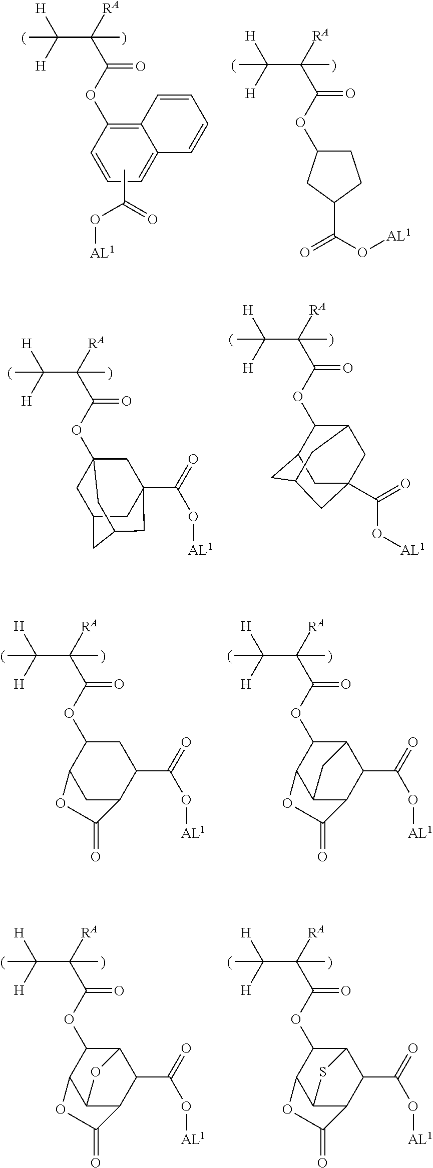

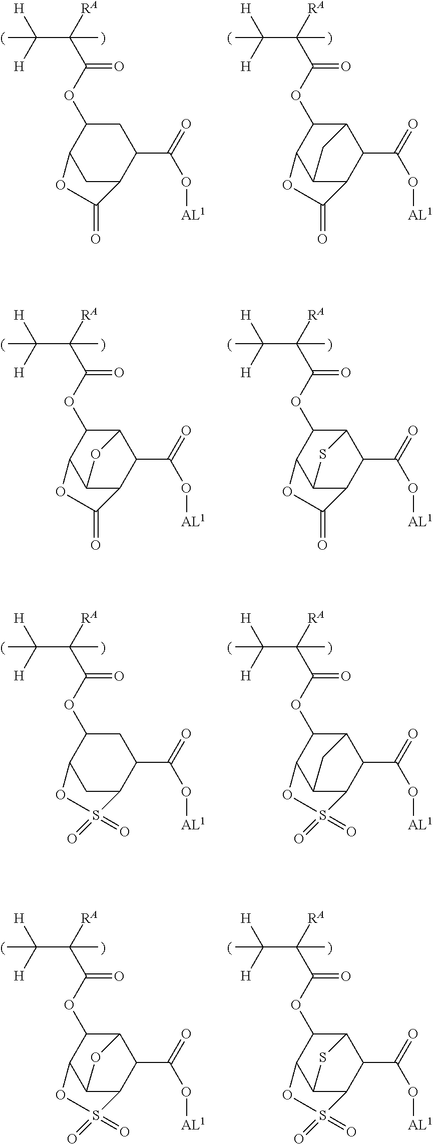

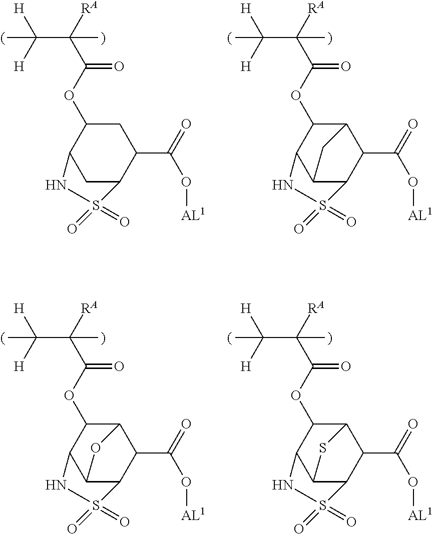

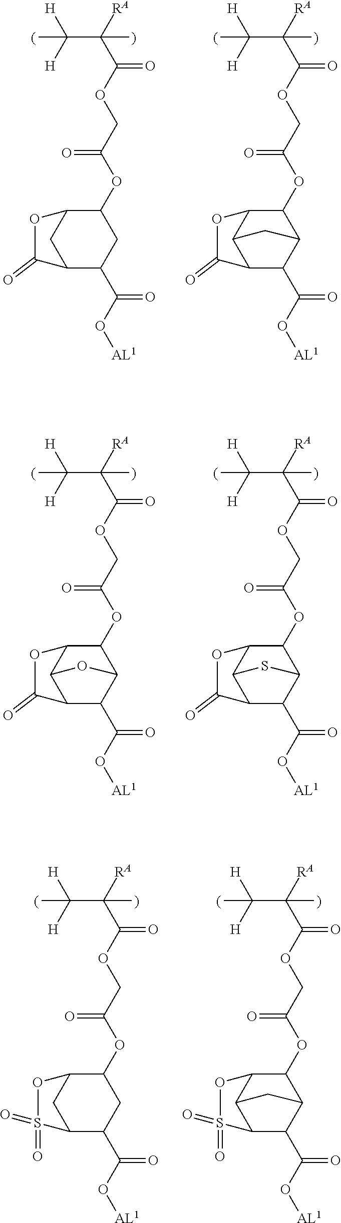

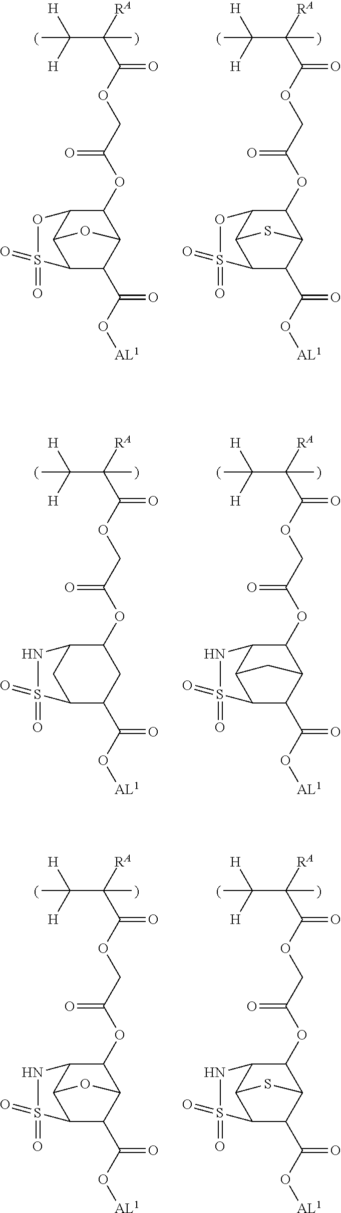

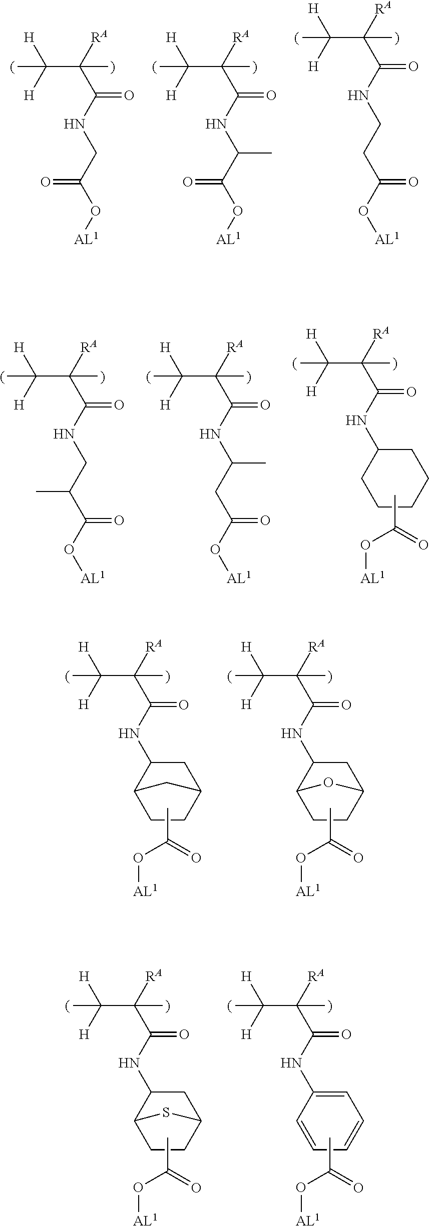

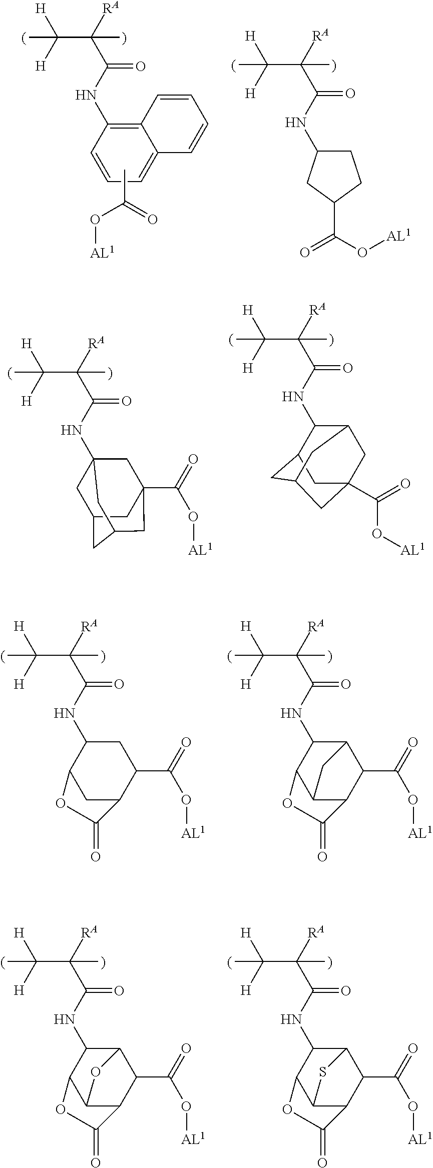

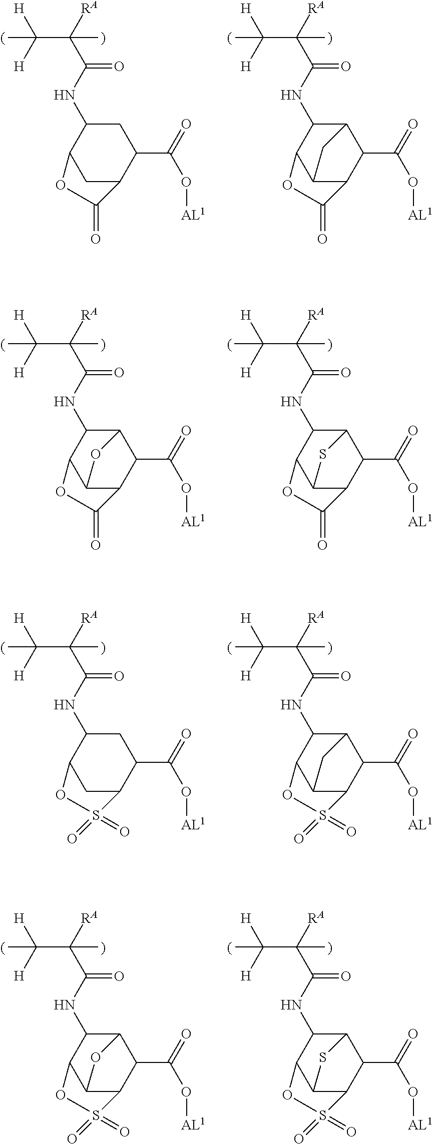

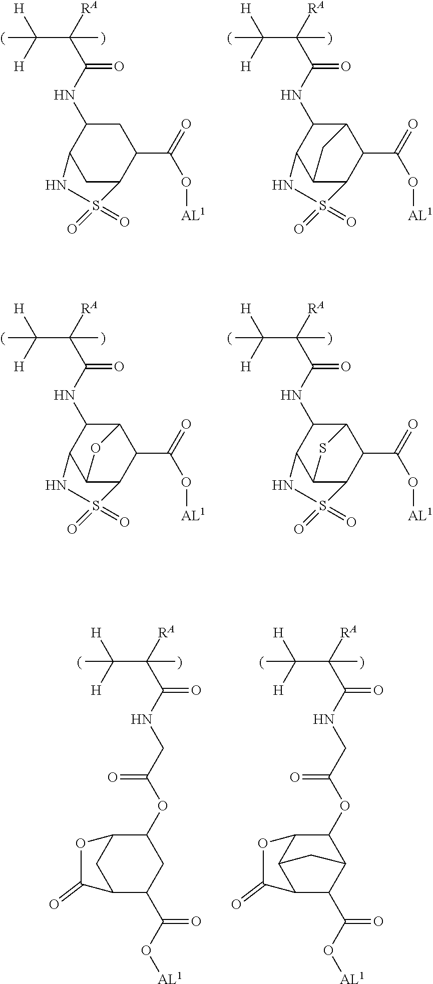

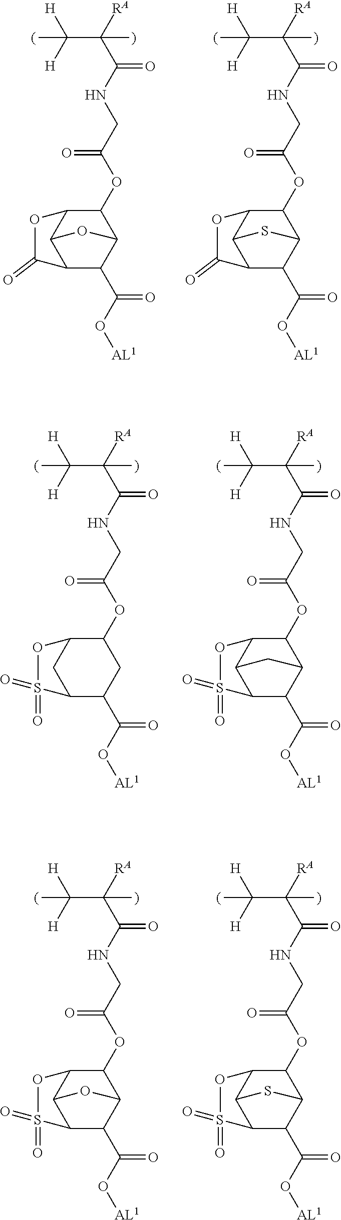

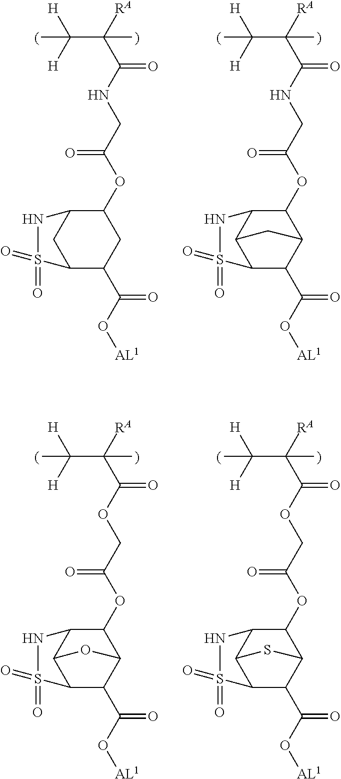

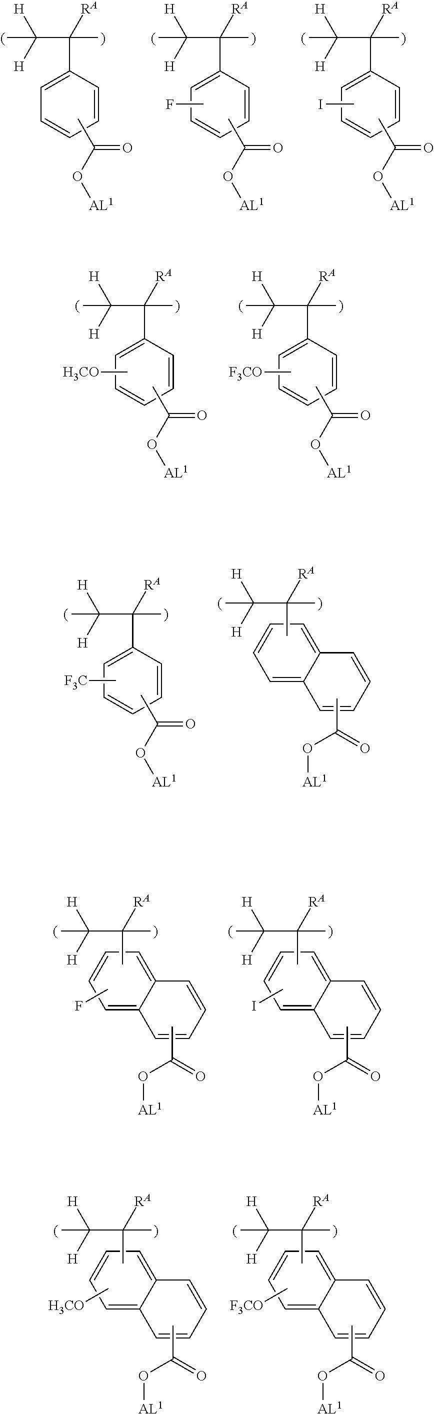

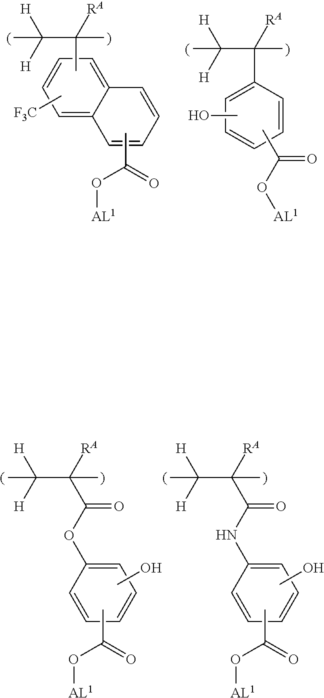

Specific examples of the repeat unit a2 include, but are not limited to, those listed below. Note that, in the following formulas, R^{A} and AL² are as defined above.

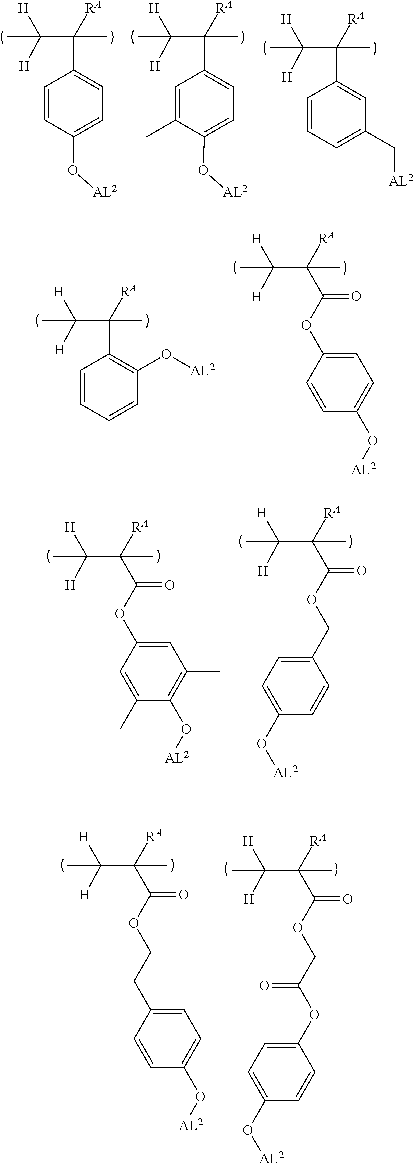

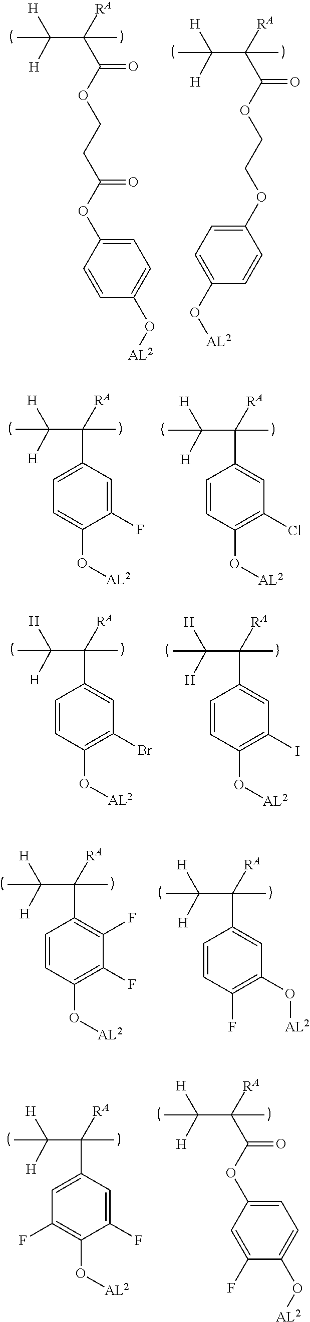

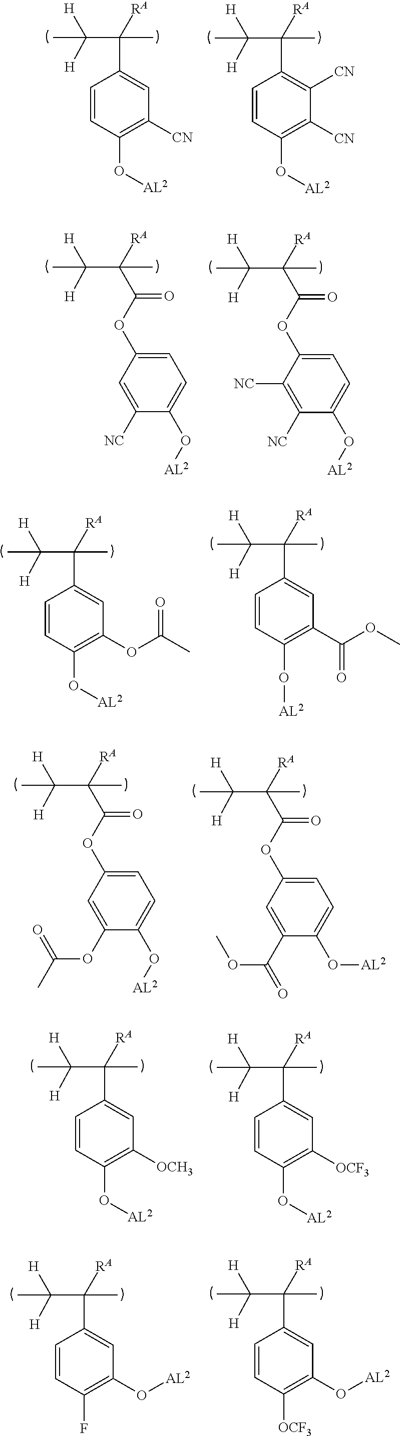

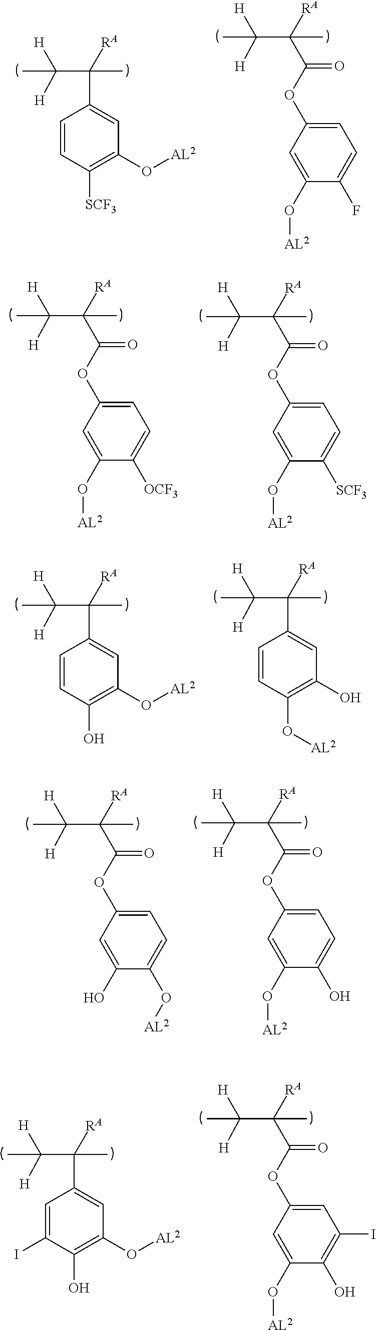

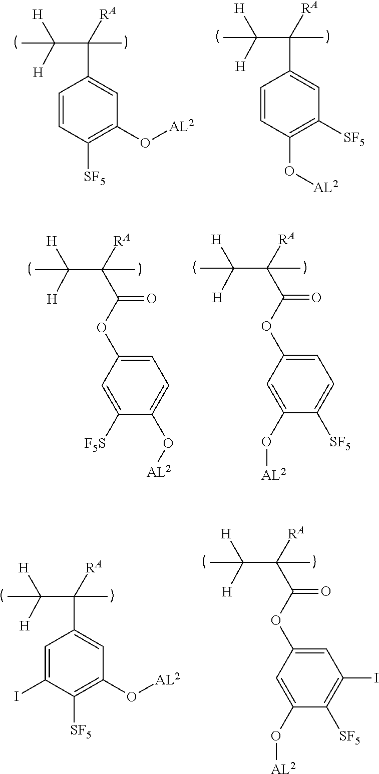

Other specific examples of the repeat unit containing an acid-unstable group include a repeat unit having the following formula (a3) (hereinafter, also referred to as a repeat unit a3).

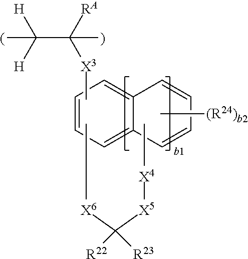

In the formula (a3), b1 is 0 or 1. When b1 is 0, it is a benzene ring, and when b1 is 1, it is a naphthalene ring, but a benzene ring in which b1 is 0 is preferable from a viewpoint of solvent solubility. When b1 is 0, b2 is 0, 1, 2, or 3, and when b1 is 1, b2 is 0, 1, 2, 3, 4, or 5. b2 is preferably 0, 1, 2, or 3, and more preferably 0, 1, or 2 from a viewpoint of raw material procurement.

In the formula (a3), R^{A} is a hydrogen atom, a fluorine atom, a methyl group, or a trifluoromethyl group. R^{A} is preferably a hydrogen atom or a methyl group, and more preferably a hydrogen atom.

In the formula (a3), X³ is a single bond, *—C(═O)—O—, or *—C(═O)—N(H)—. * represents a bond with a carbon atom of a main chain. Among these, X³ is preferably a single bond or *—C(═O)—O—, and more preferably a single bond.

In the formula (a3), X⁴ is a single bond, a C₁-C₄ aliphatic hydrocarbylene group, a carbonyl group, a sulfonyl group, or a group obtained by combining these groups. Among these, X⁴ is preferably a single bond, a carbonyl group, or a sulfonyl group from a viewpoint of raw material procurement, and is more preferably a single bond or a carbonyl group from a viewpoint of a polar group generated after reaction.

In the formula (a3), X⁵ and X⁶ are each independently an oxygen atom or a sulfur atom. Provided that X⁴ and X⁶ are bonded to adjacent aromatic ring carbon atoms. X⁵ and X⁶ may be the same as or different from each other, but X⁵ and X⁶ are both preferably oxygen atoms from a viewpoint of reactivity.

In the formula (a3), R²² and R²³ are each independently a hydrogen atom or a C₁-C₂₀ hydrocarbyl group which may contain a heteroatom. The hydrocarbyl group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, or an icosyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₂₀ alkenyl group such as a vinyl group, a 1-propenyl group, a 2-propenyl group, a butenyl group, or a hexenyl group; a C₃-C₂₀ cyclic unsaturated hydrocarbyl group such as a cyclohexenyl group; a C₆-C₂₀ aryl group such as a phenyl group or a naphthyl group; a C₇-C₂₀ aralkyl group such as a benzyl group, a 1-phenylethyl group, or a 2-phenylethyl group; and a group obtained by combining these groups. Some or all of the hydrogen atoms of the hydrocarbyl group may be replaced with a group containing a heteroatom such as an oxygen atom, a sulfur atom, a nitrogen atom, or a halogen atom, some of the (—CH₂-)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

R²² and R²³ may be bonded to each other to form a ring together with a carbon atom to which they are bonded. Specific examples of the ring formed at this time include a cyclopropane ring, a cyclobutane ring, a cyclopentane ring, a cyclohexane ring, a norbornane ring, and an adamantane ring. Some or all of the hydrogen atoms of the ring may be replaced with a group containing a heteroatom such as an oxygen atom, a sulfur atom, a nitrogen atom, or a halogen atom, some of the (—CH₂-)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

In the formula (a3), R²⁴ is a halogen atom, a hydroxy group, a cyano group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}). R^{24A} and R^{24B} are each independently a hydrogen atom or a C₁-C₆ hydrocarbyl group. The halogen atom is preferably a fluorine atom, a chlorine atom, a bromine atom, or an iodine atom, and more preferably a fluorine atom or an iodine atom. The hydrocarbyl group and hydrocarbyl moieties of the hydrocarbyloxy group, the hydrocarbyloxycarbonyl group, and the hydrocarbylthio group may be saturated or unsaturated, and may be linear, branched, or cyclic. Specific examples thereof include those similar to those exemplified as the hydrocarbyl group of R²² and R²³. Some or all of the hydrogen atoms of the hydrocarbyl group may be replaced with a group containing a heteroatom such as an oxygen atom, a sulfur atom, a nitrogen atom, or a halogen atom, some of the (—CH₂-)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like. When b2 is 2 or more, R²⁴s may be the same as or different from each other.

When b2 is 2 or more, a plurality of R²⁴s may be bonded to each other to form a ring together with aromatic ring carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of the hydrogen atoms of the ring may be replaced with a group containing a heteroatom such as an oxygen atom, a sulfur atom, a nitrogen atom, or a halogen atom, some of the (—CH₂-)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

Specific examples of the repeat unit a3 include, but are not limited to, those listed below. Note that, in the following formulas, R^{A} is as defined above, and Me is a methyl group. Bonding positions of various substituents on an aromatic ring may be replaced with each other.

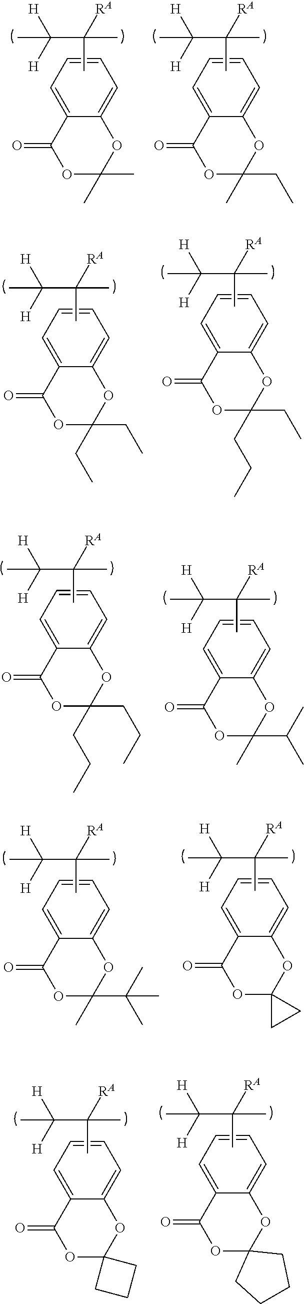

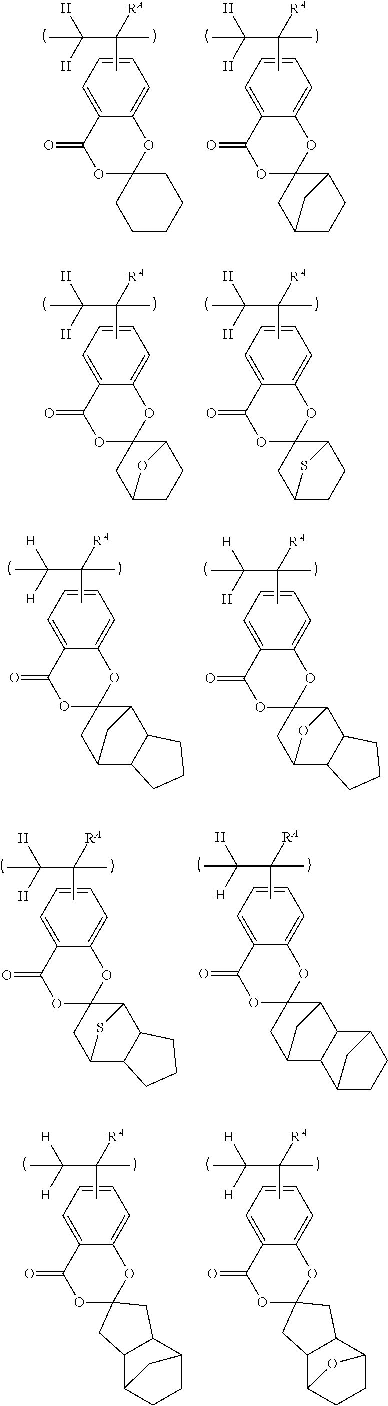

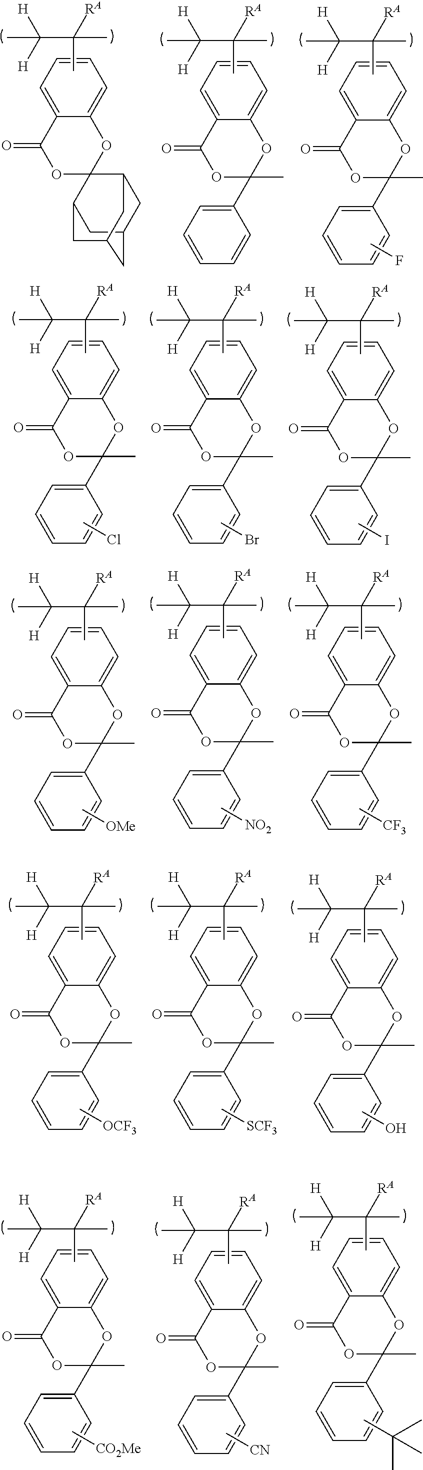

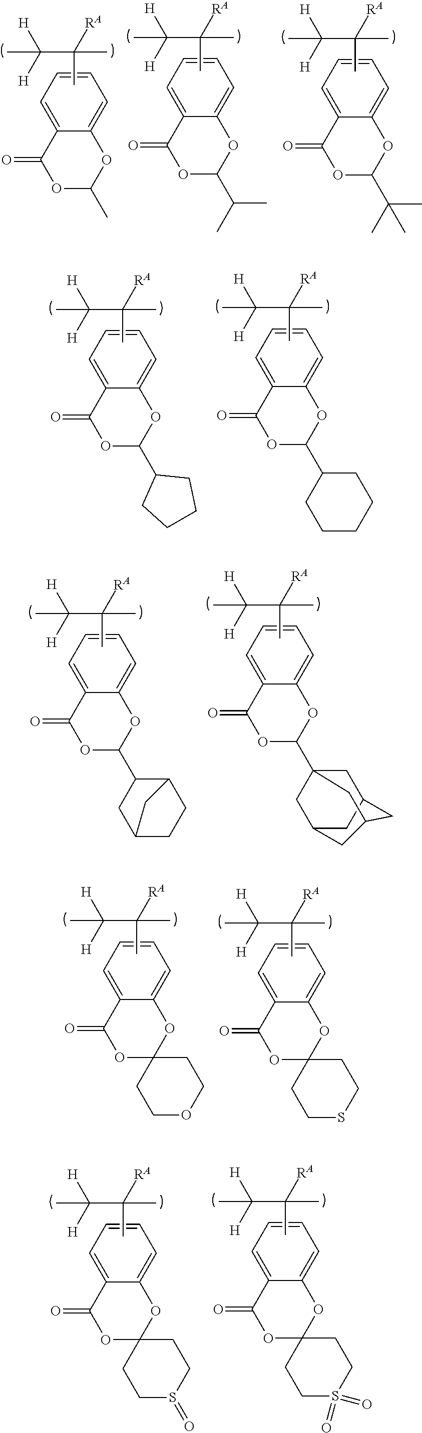

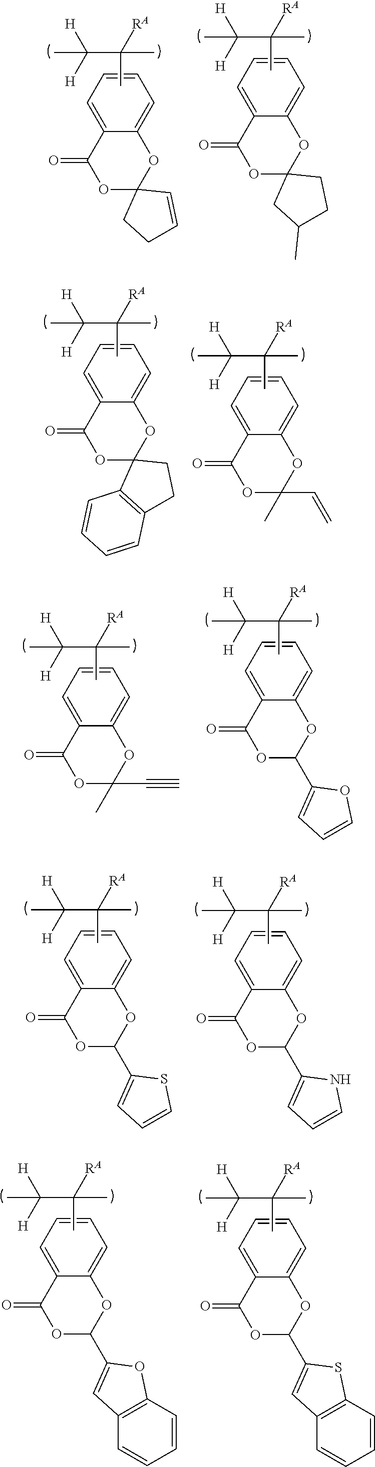

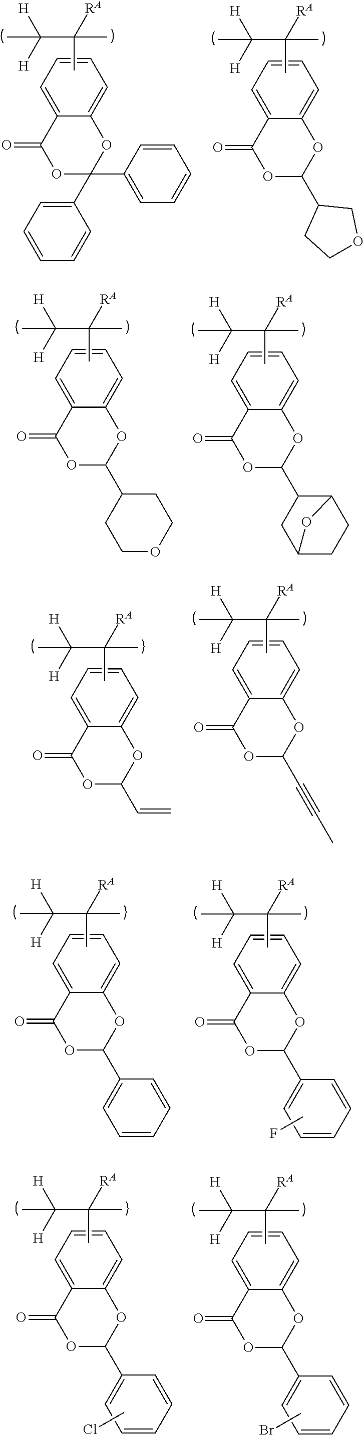

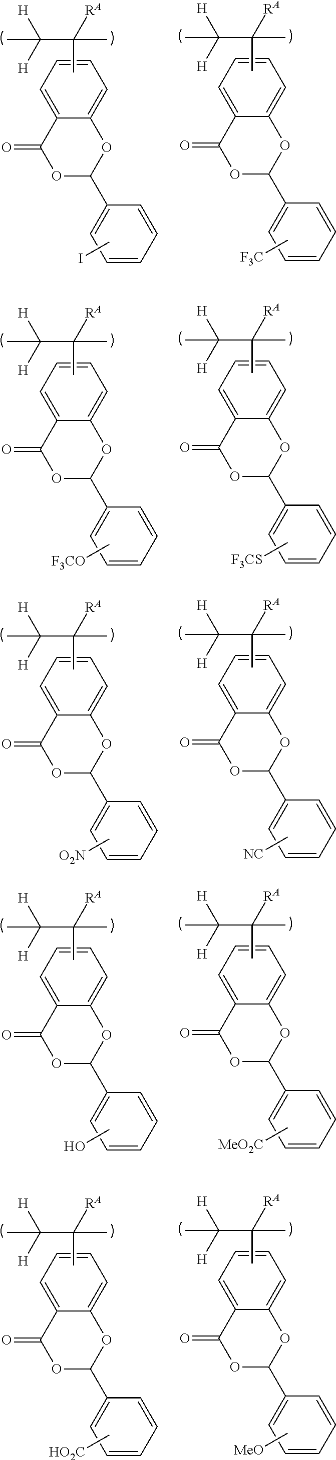

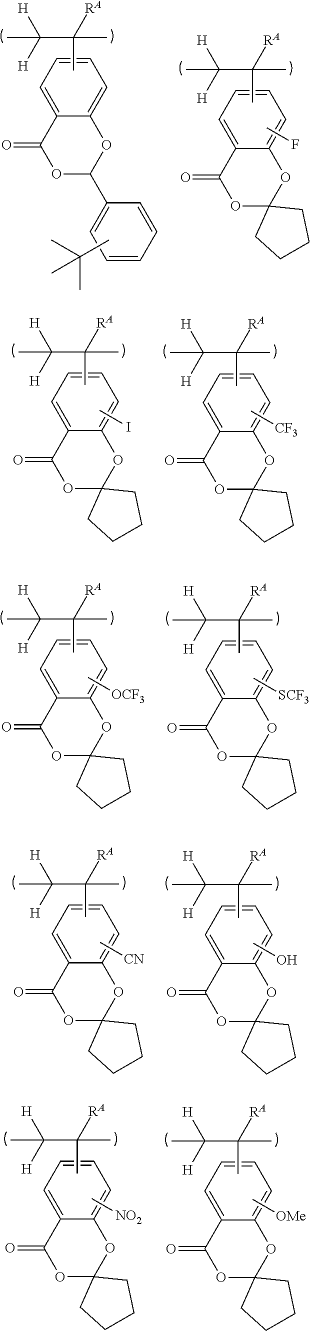

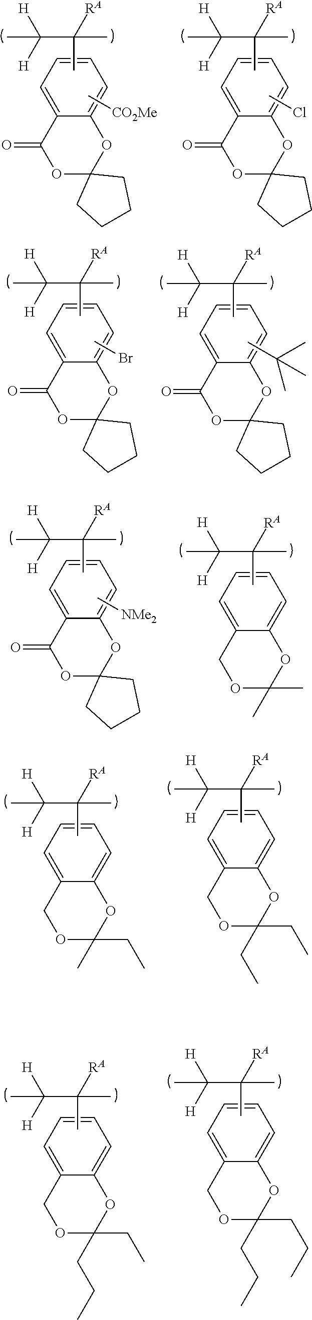

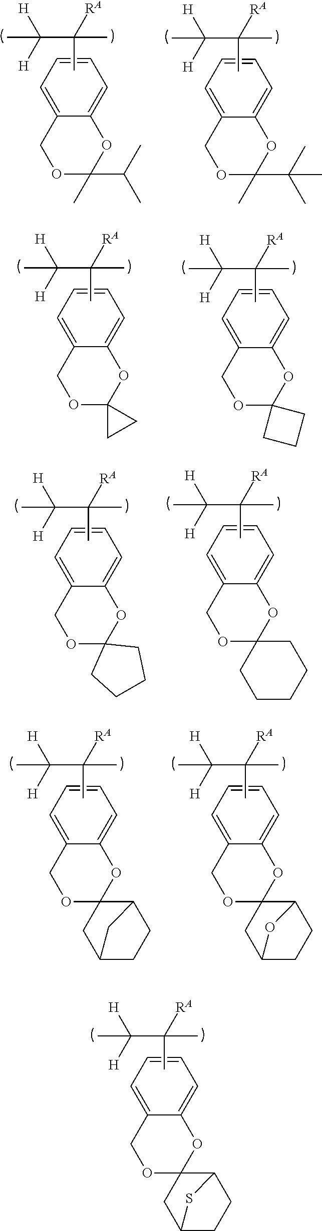

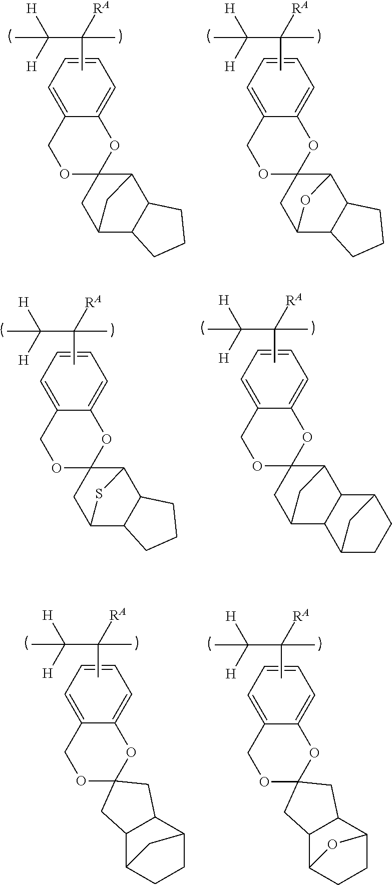

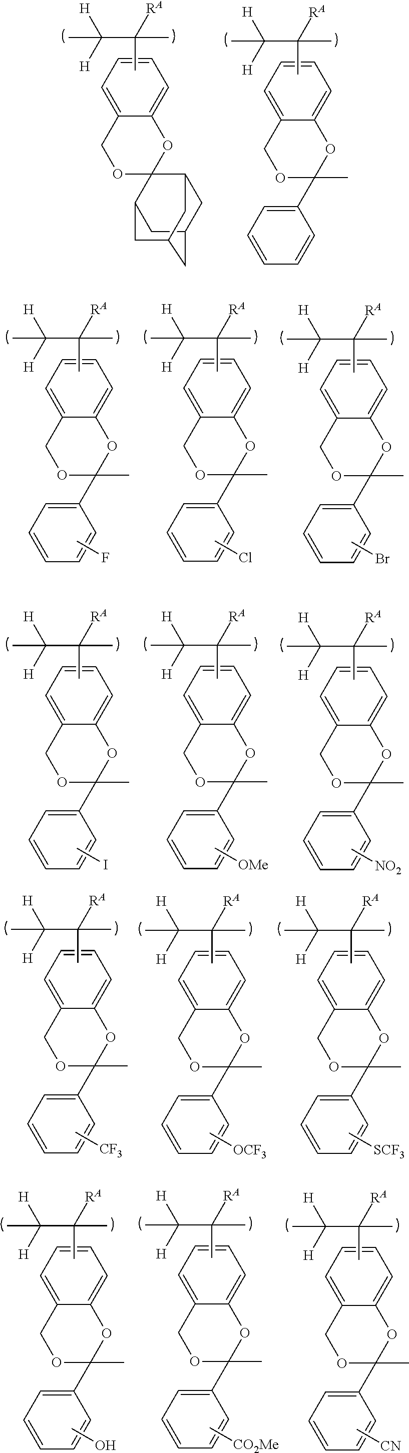

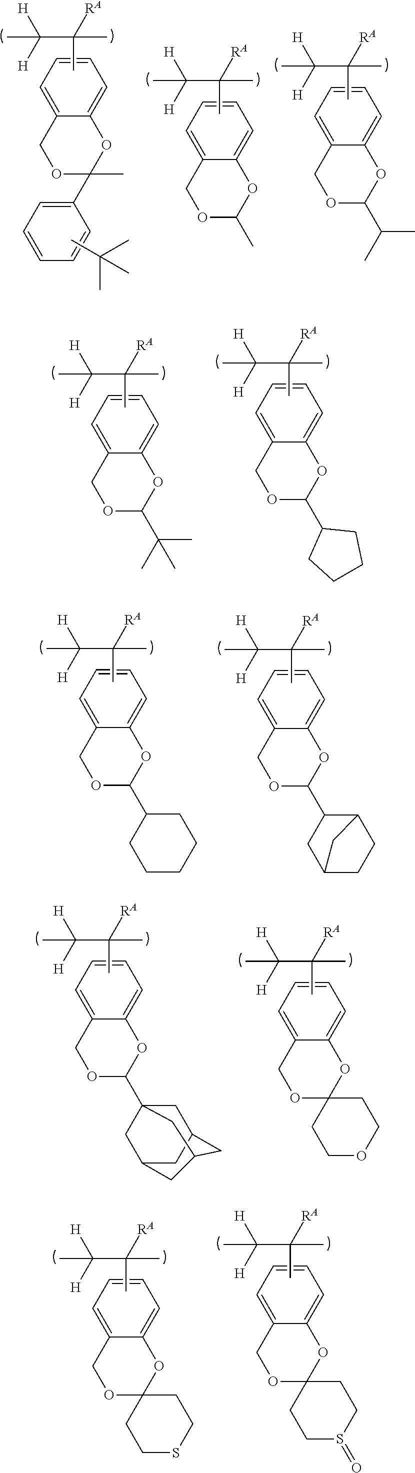

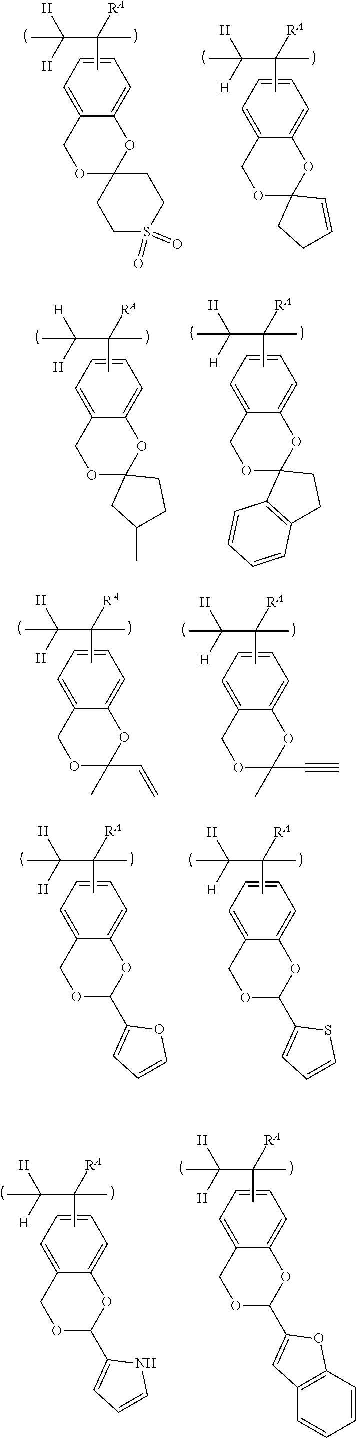

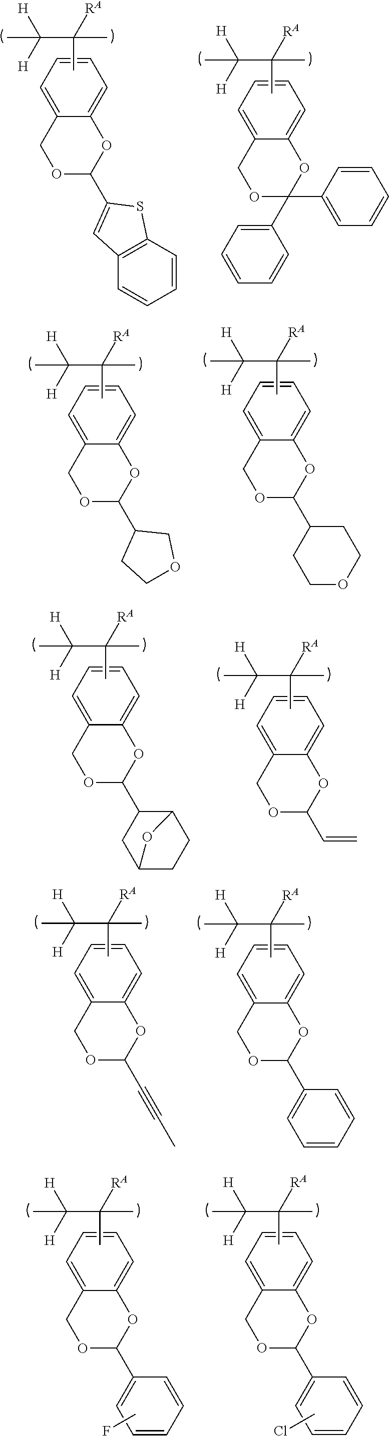

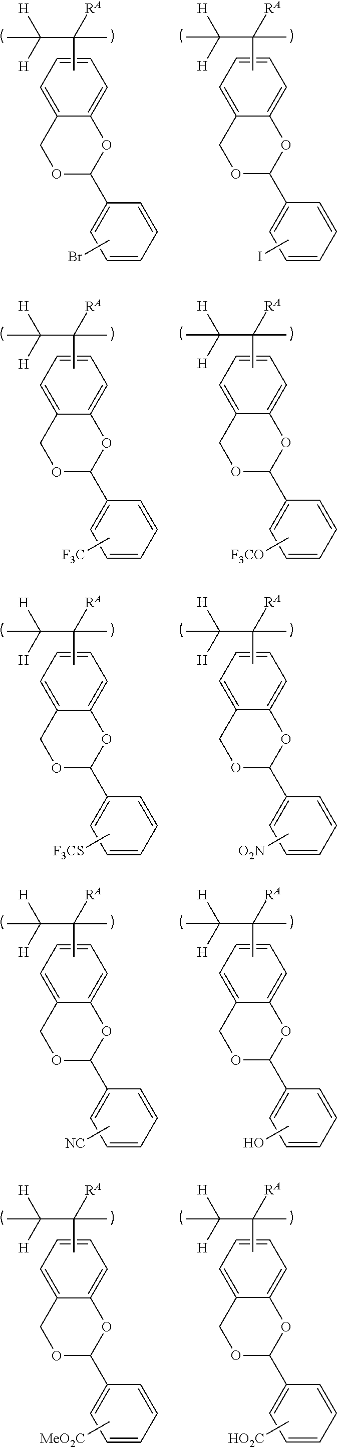

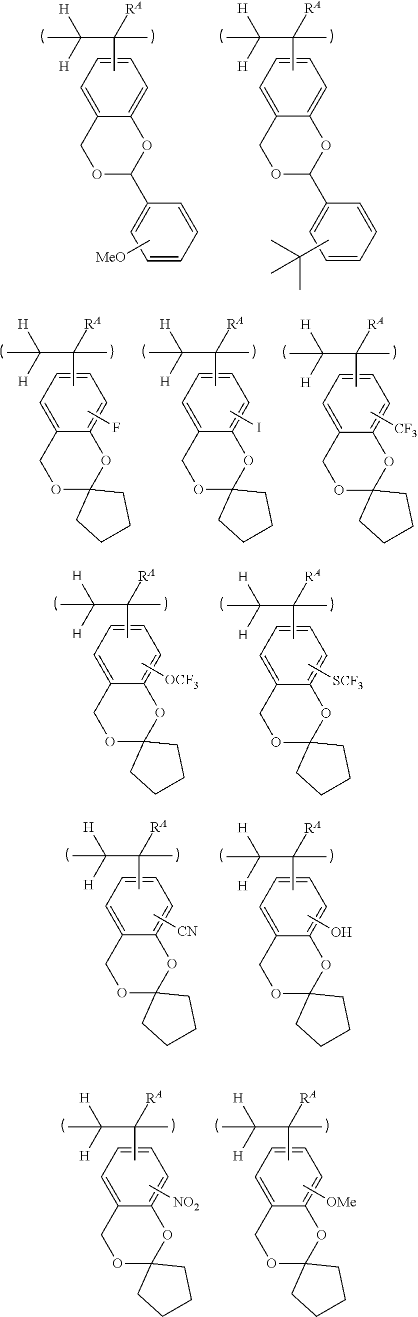

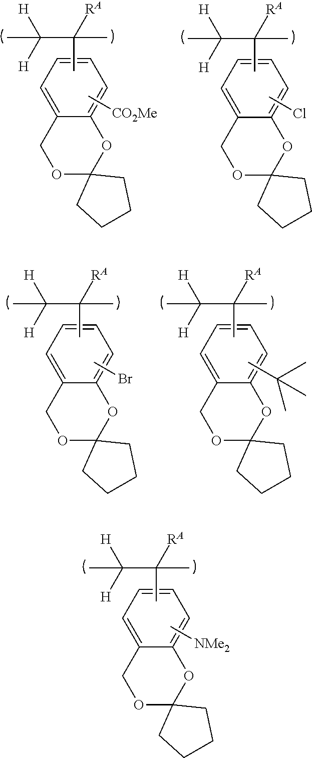

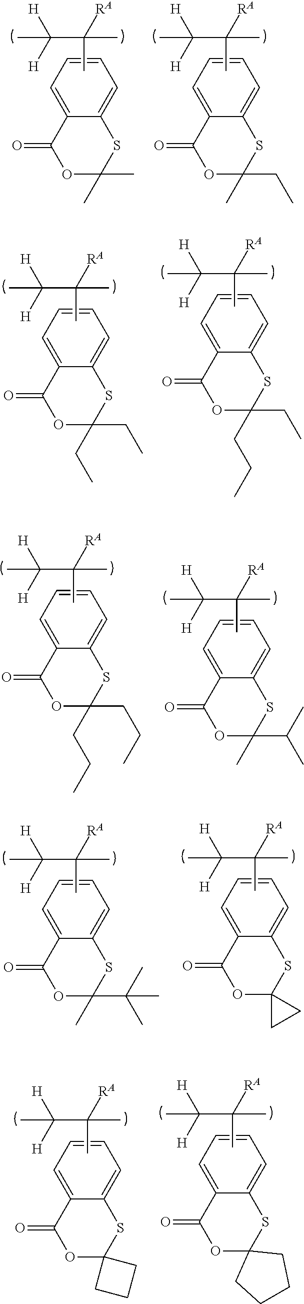

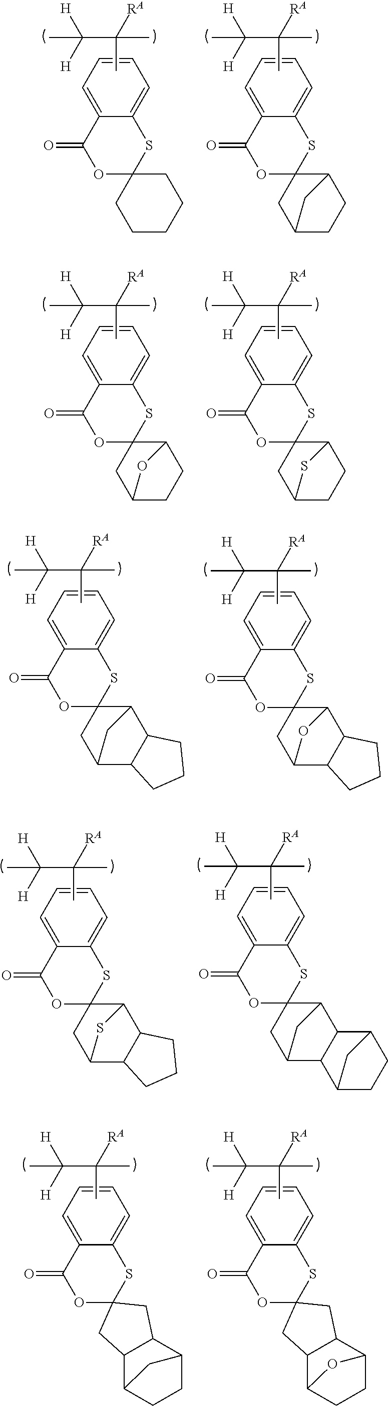

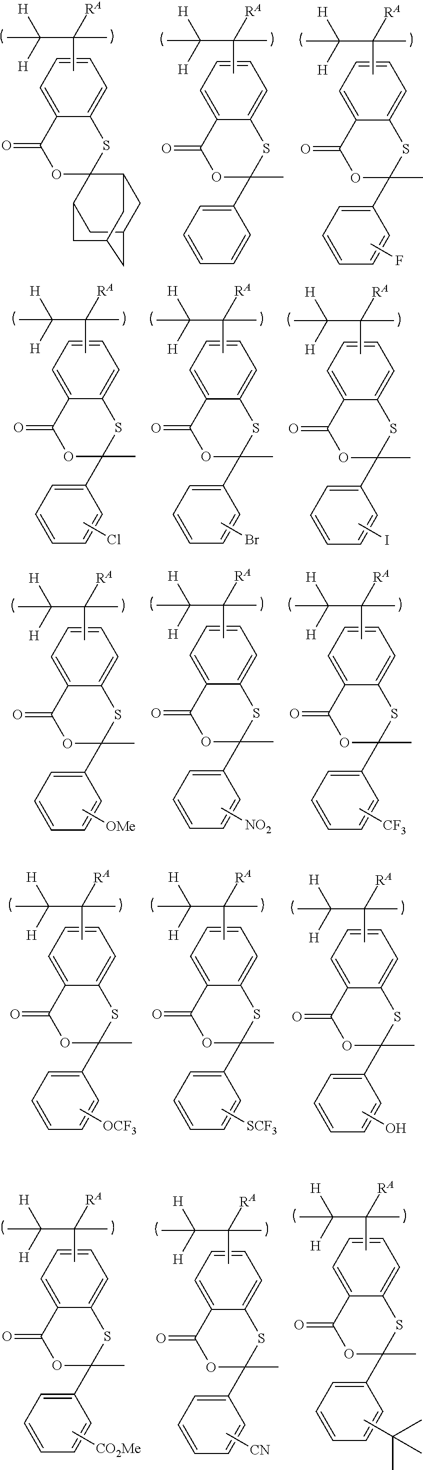

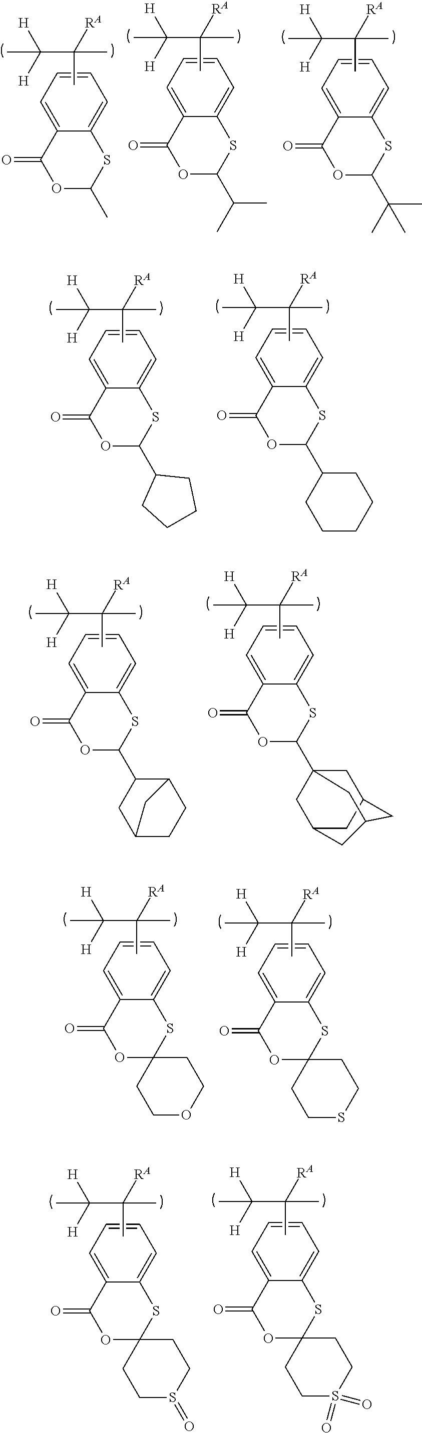

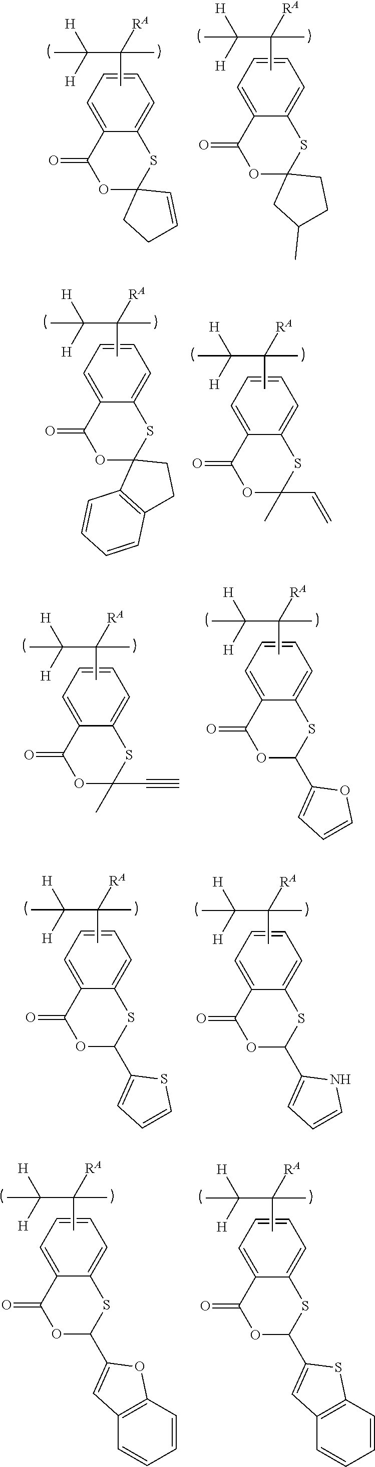

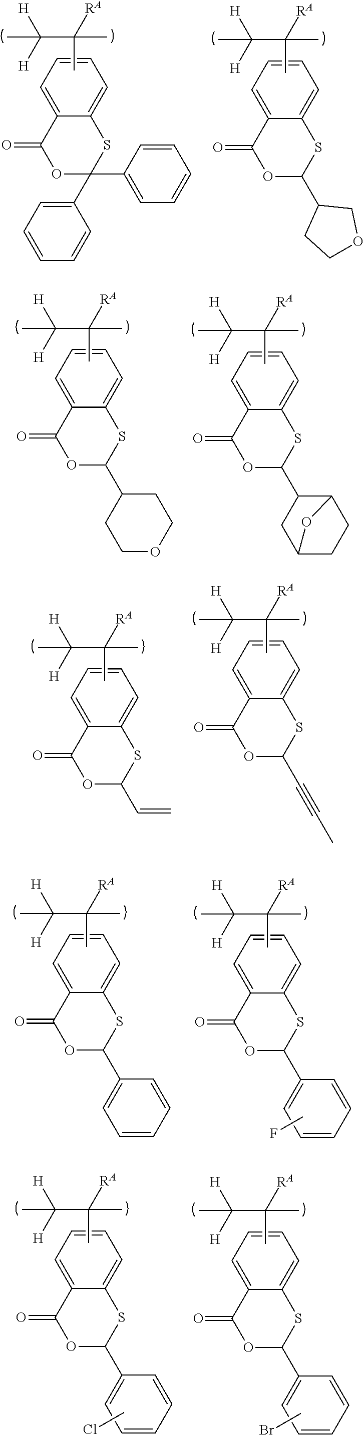

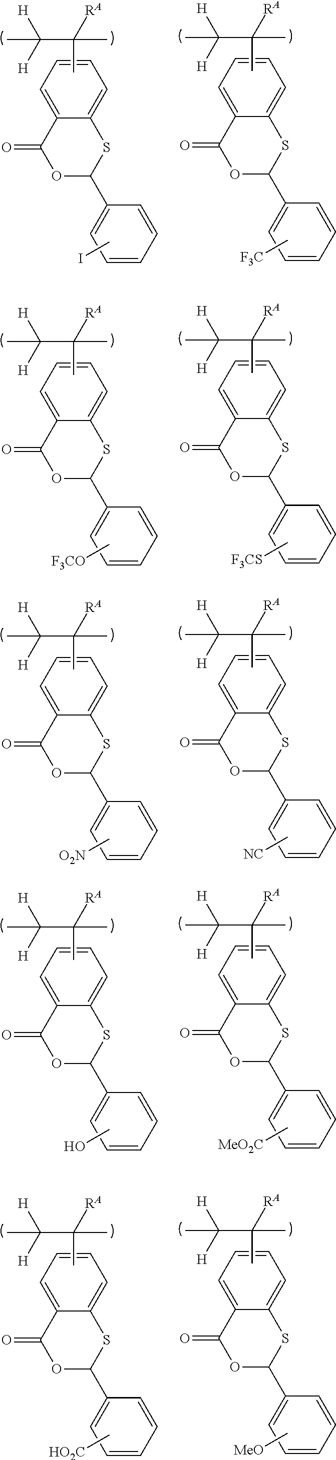

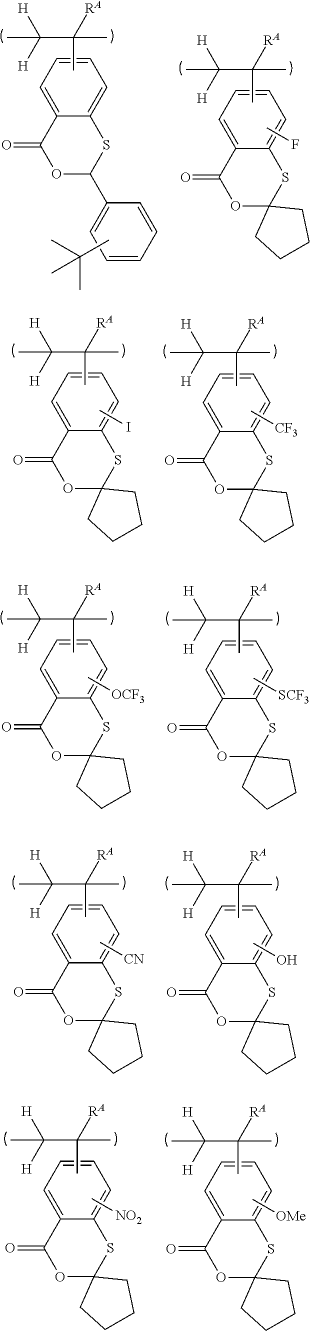

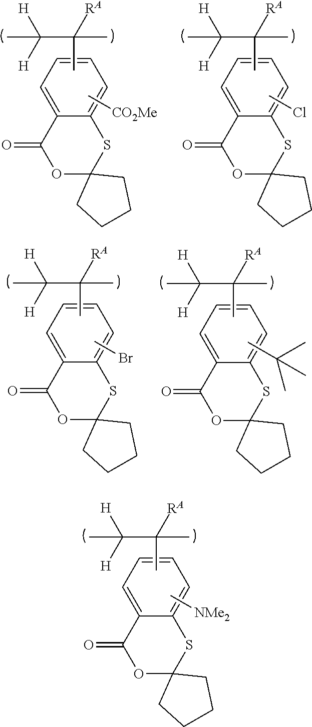

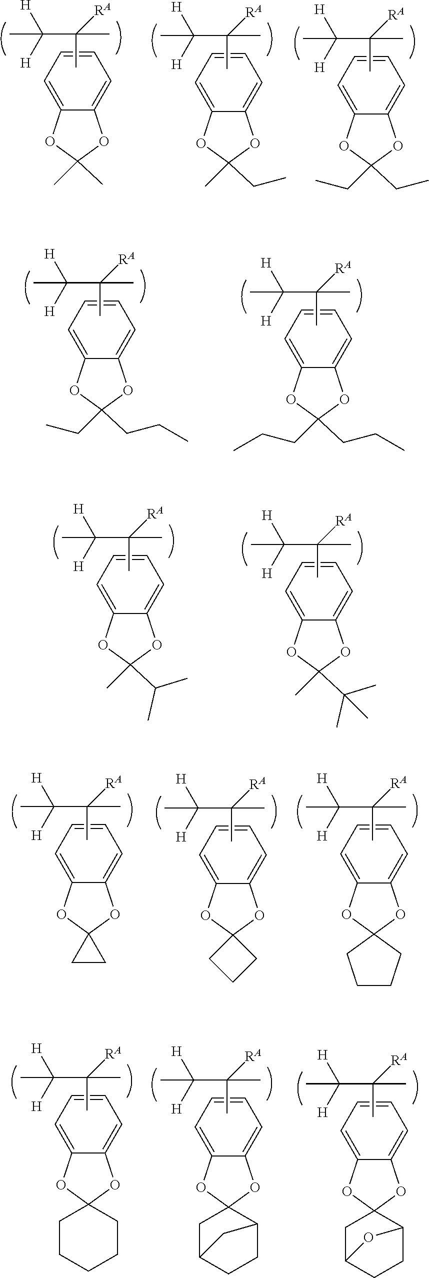

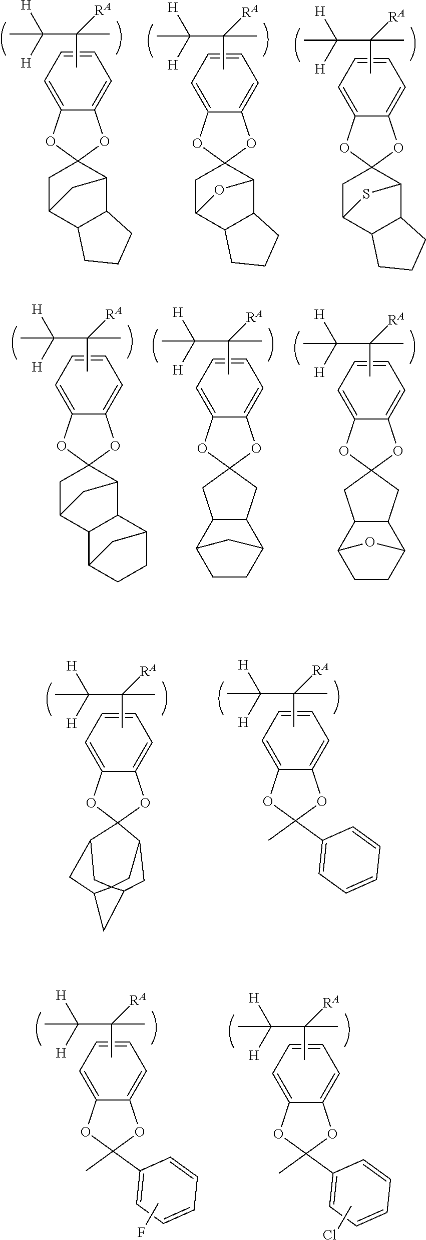

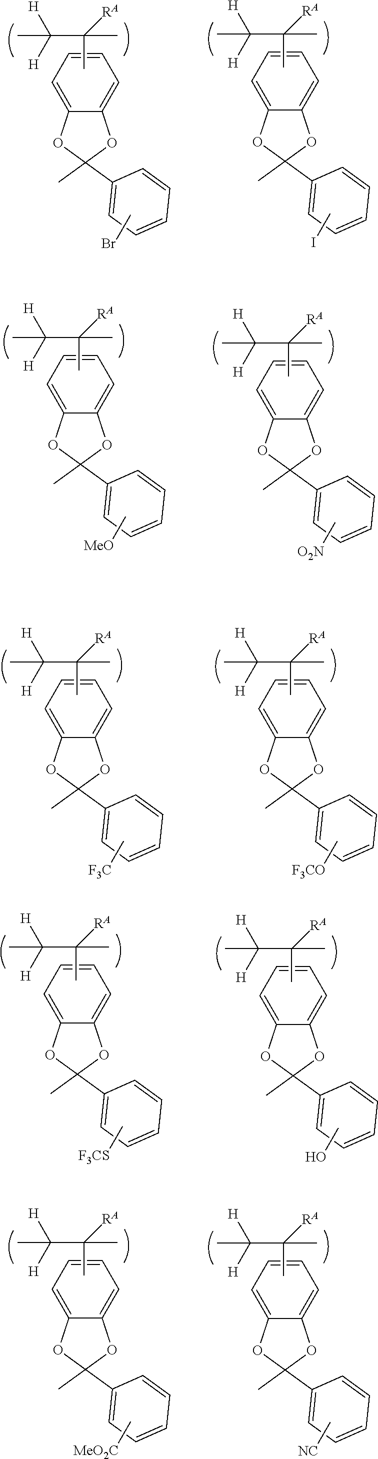

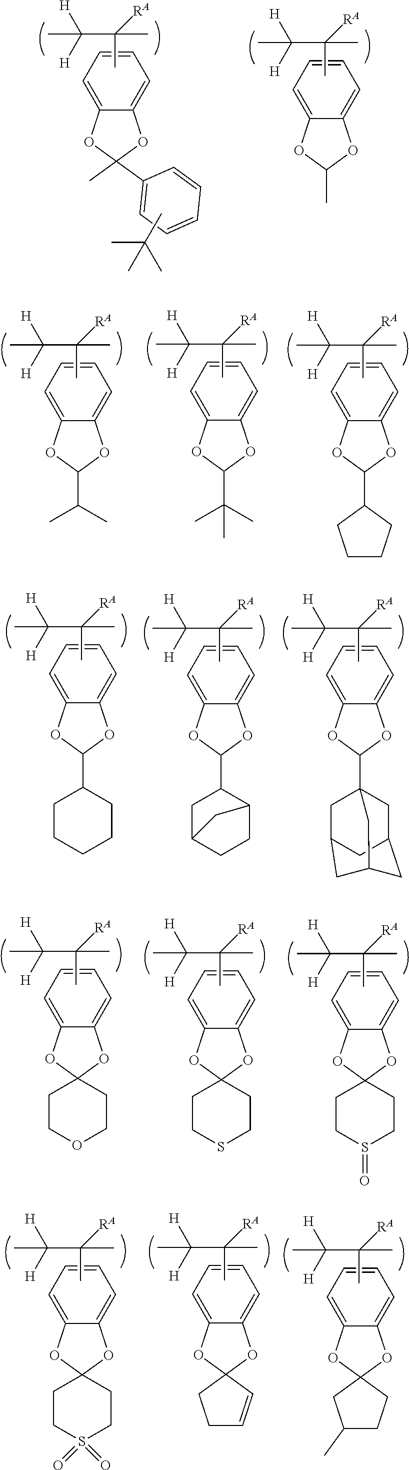

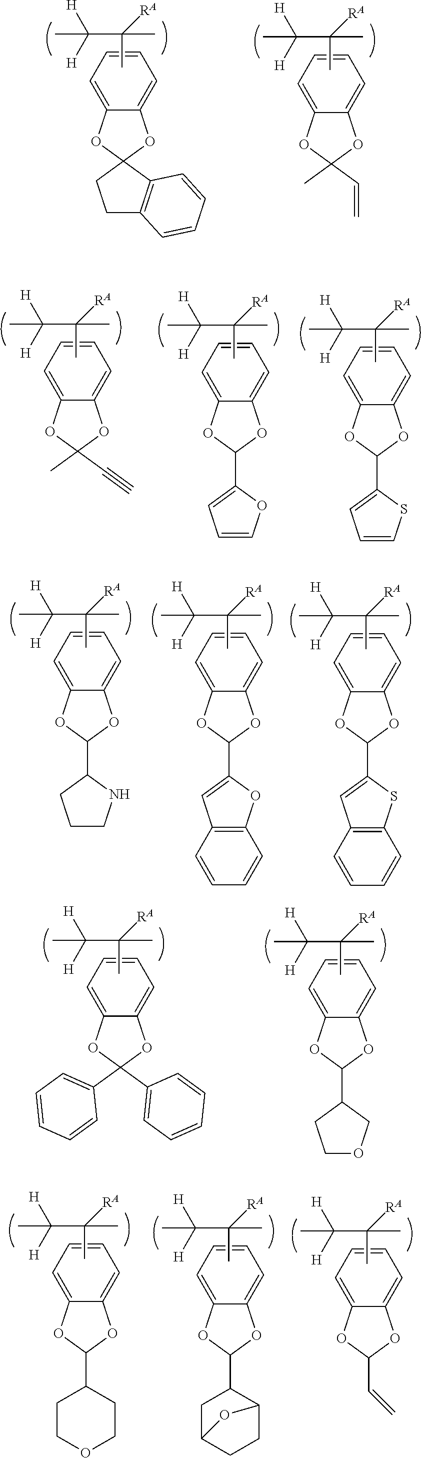

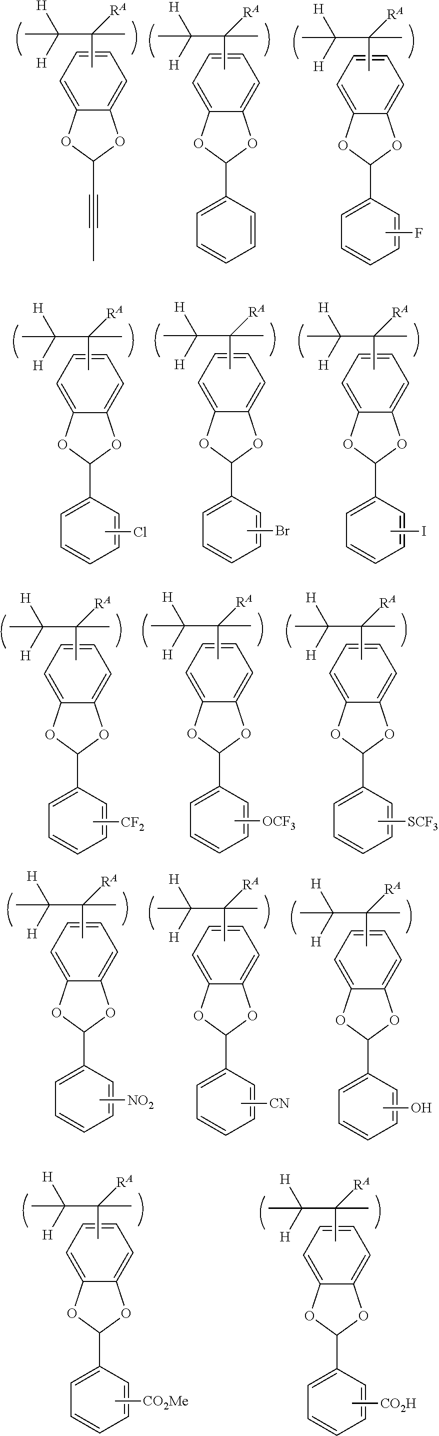

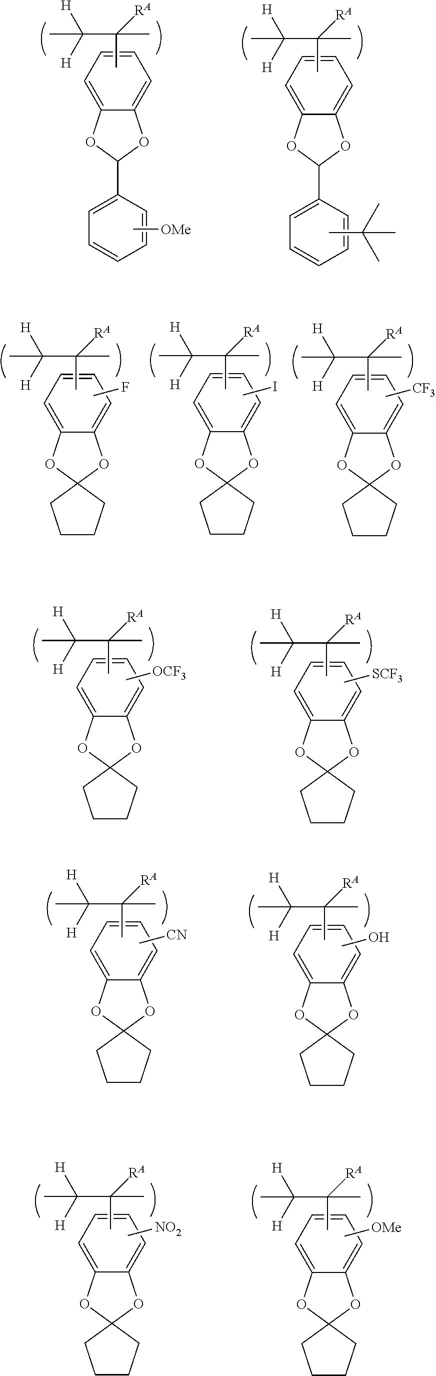

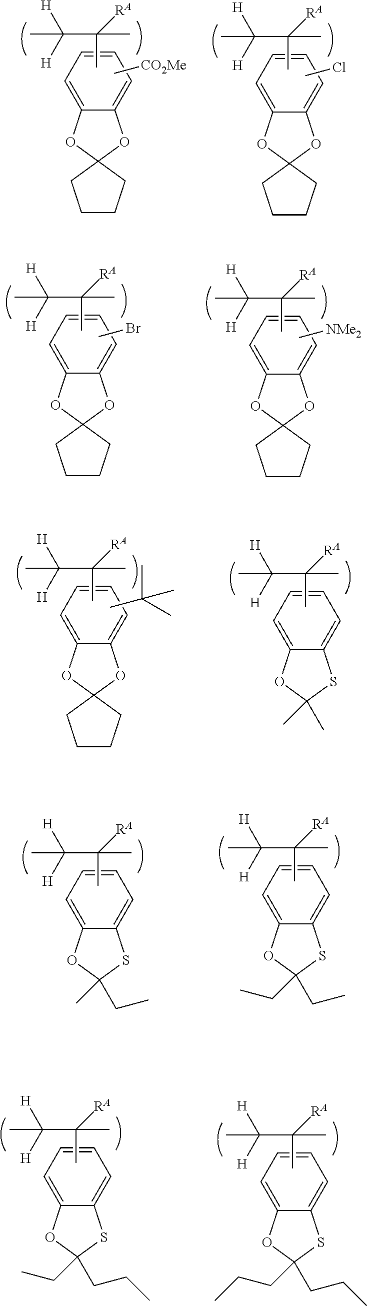

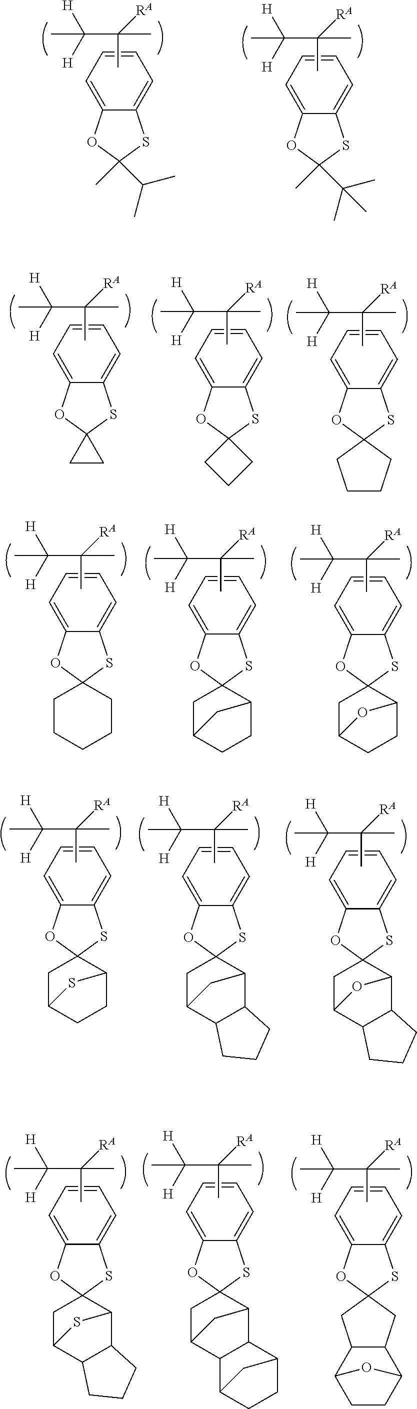

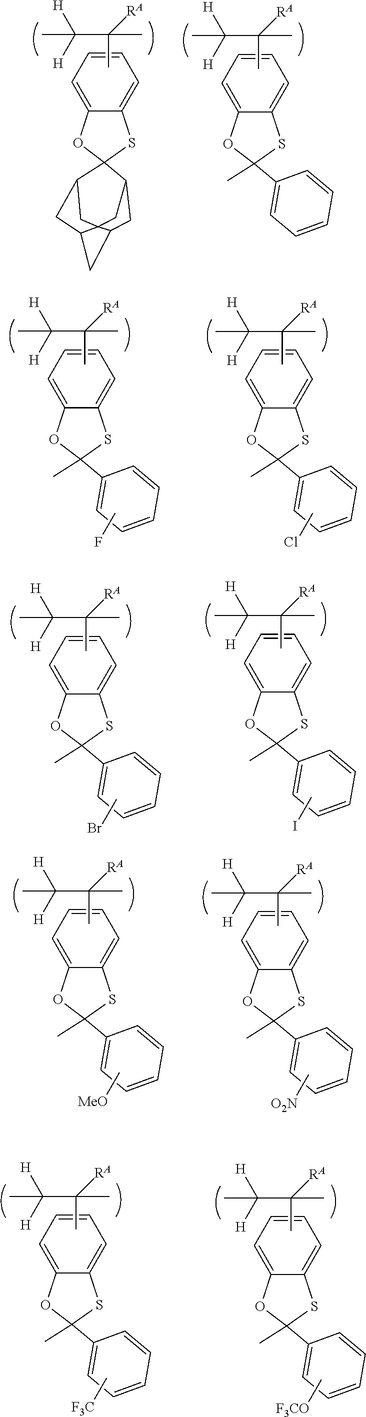

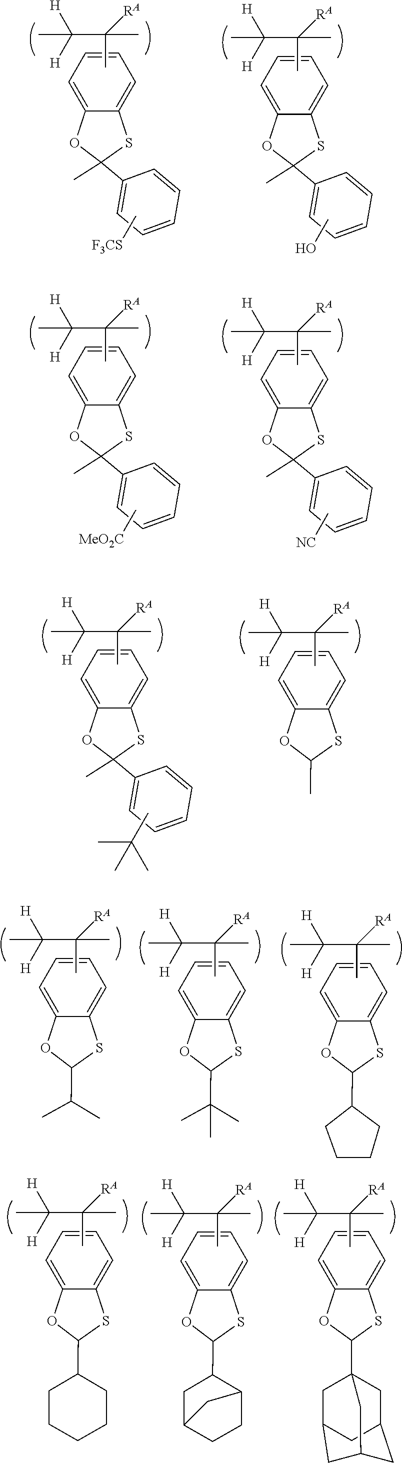

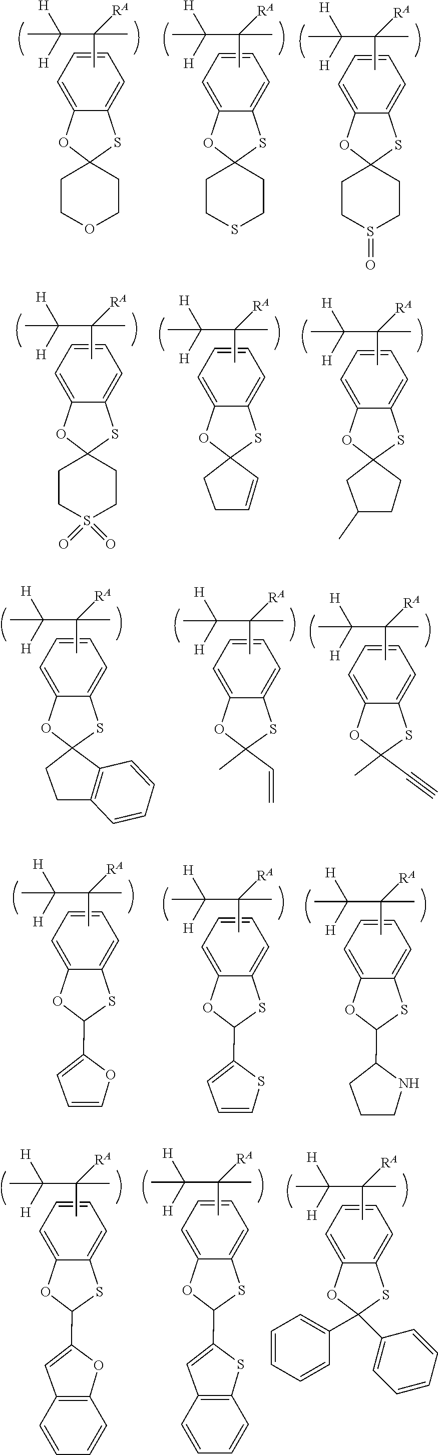

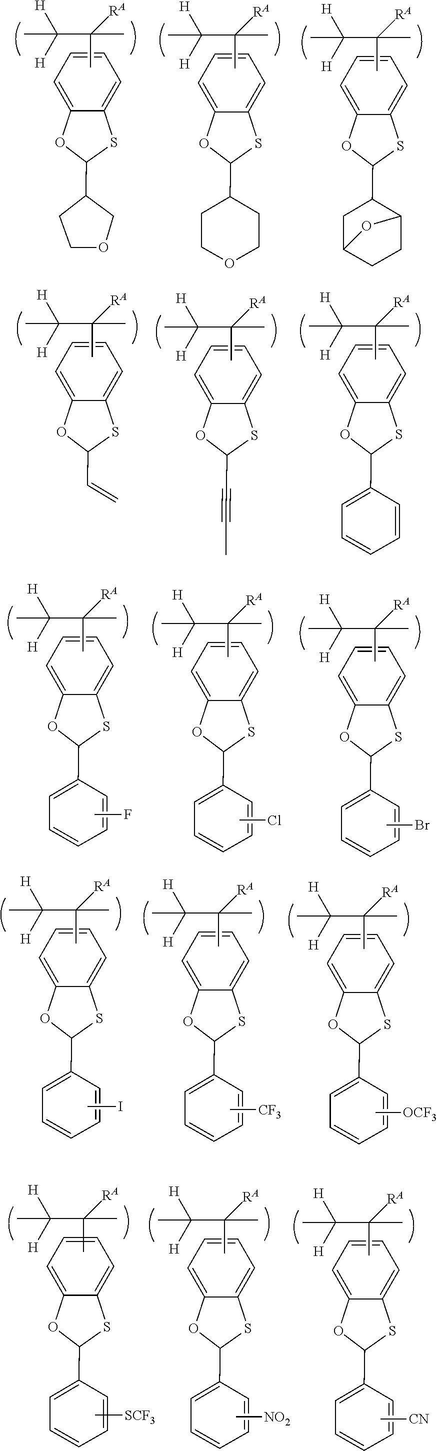

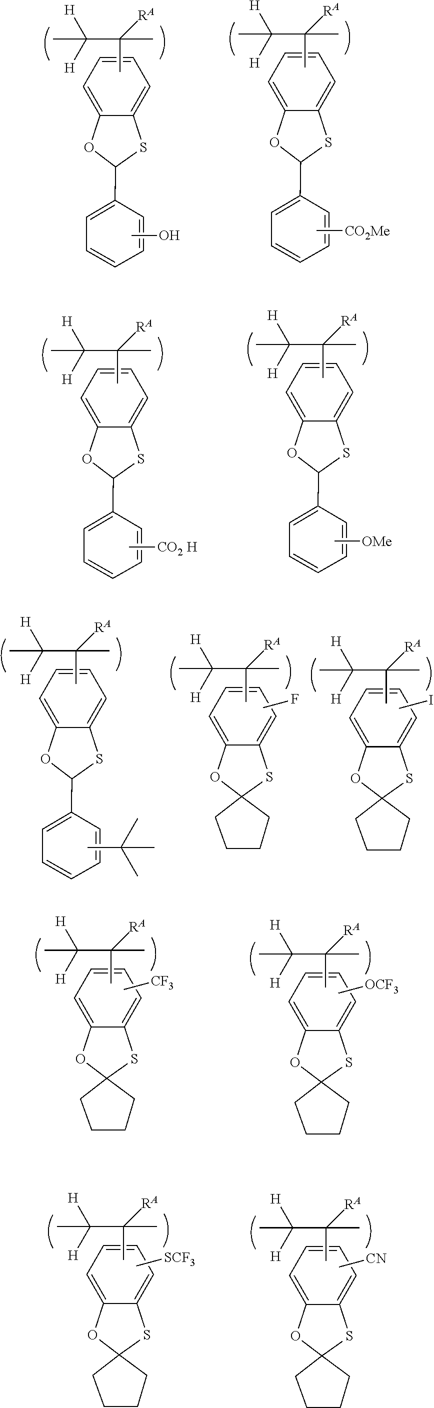

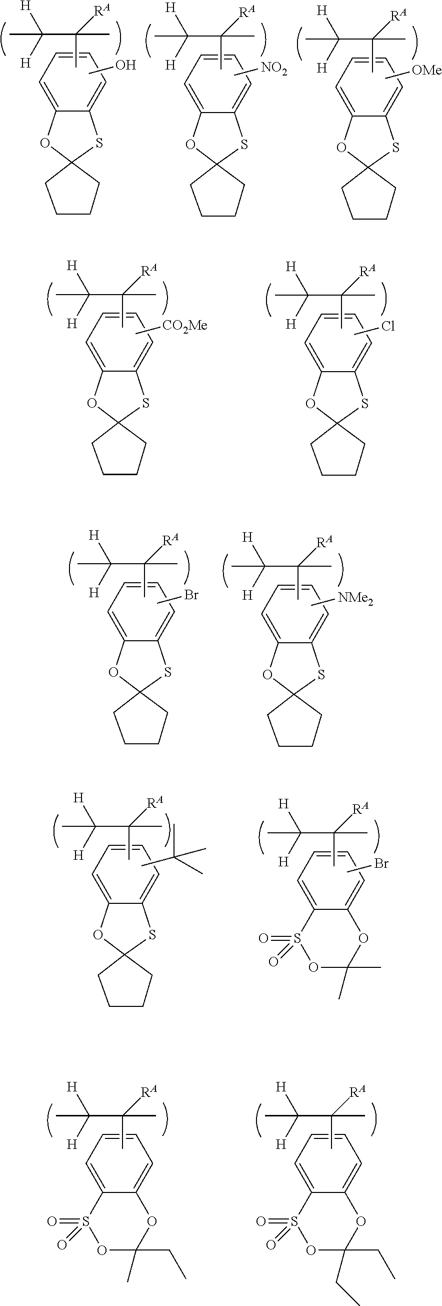

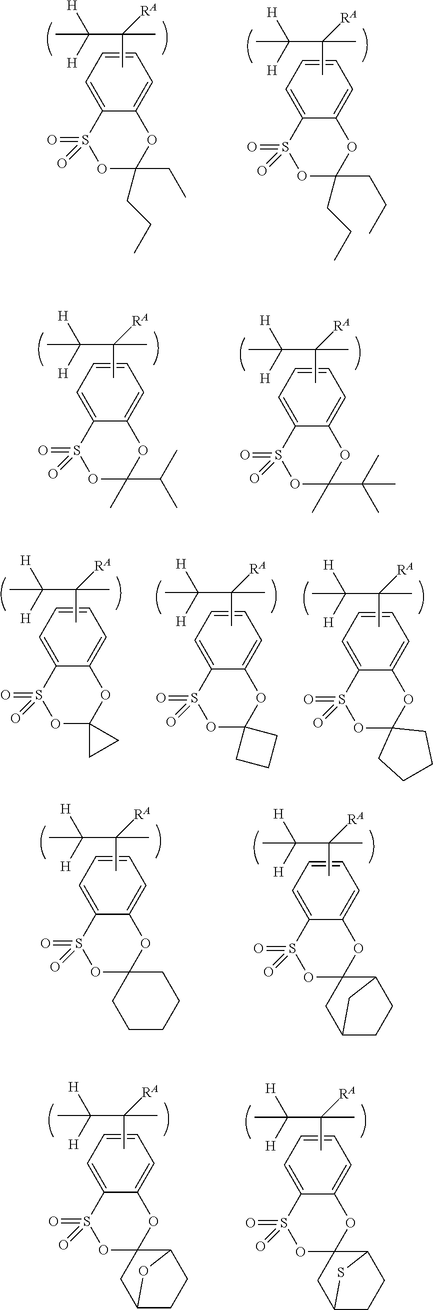

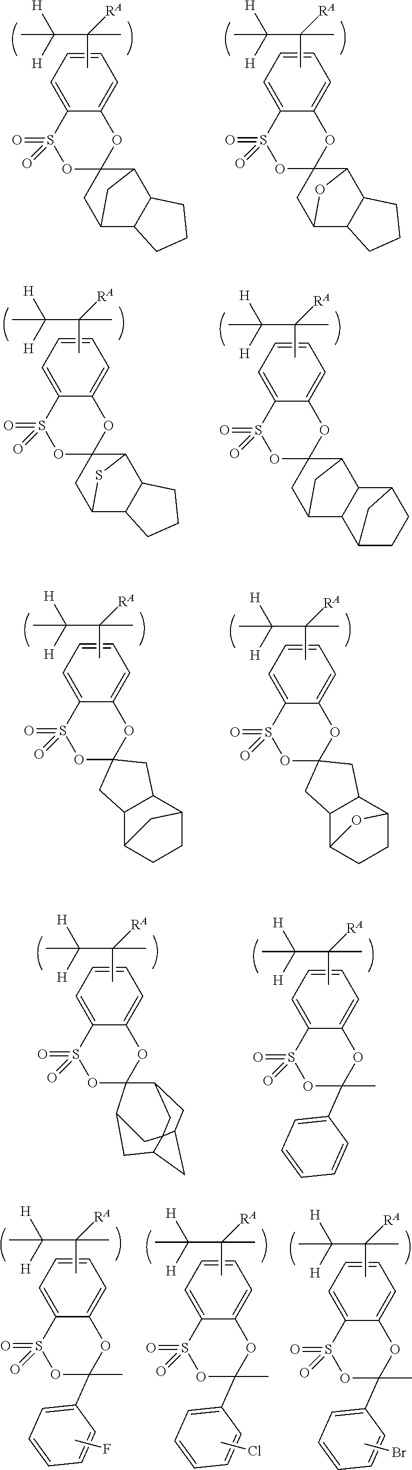

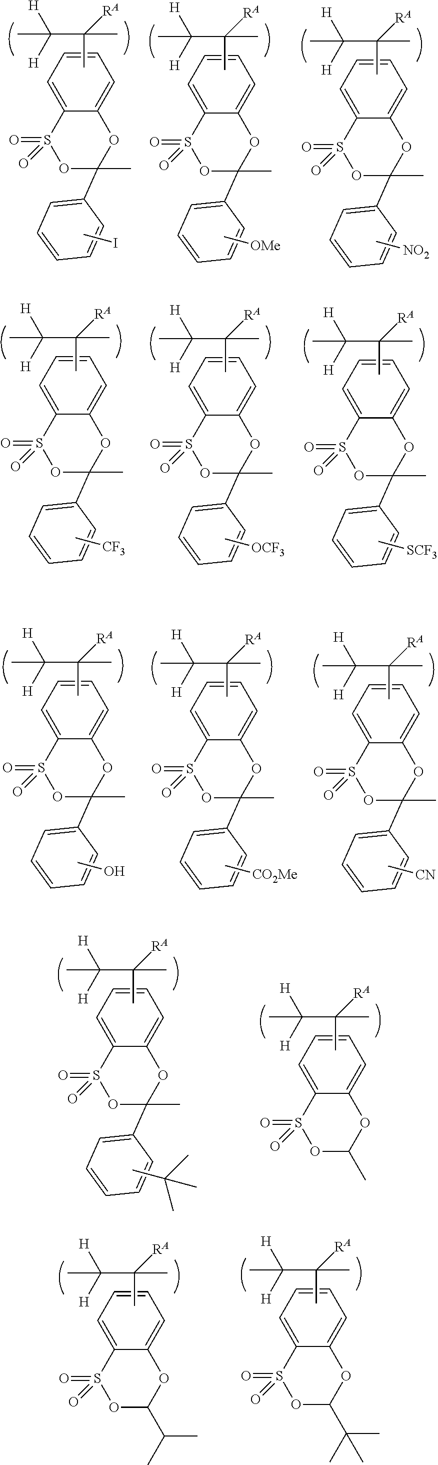

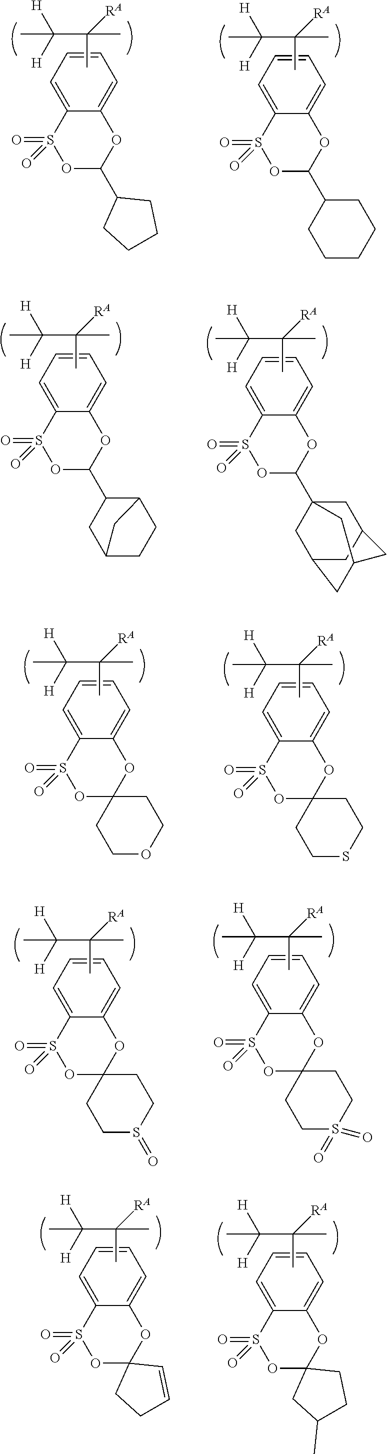

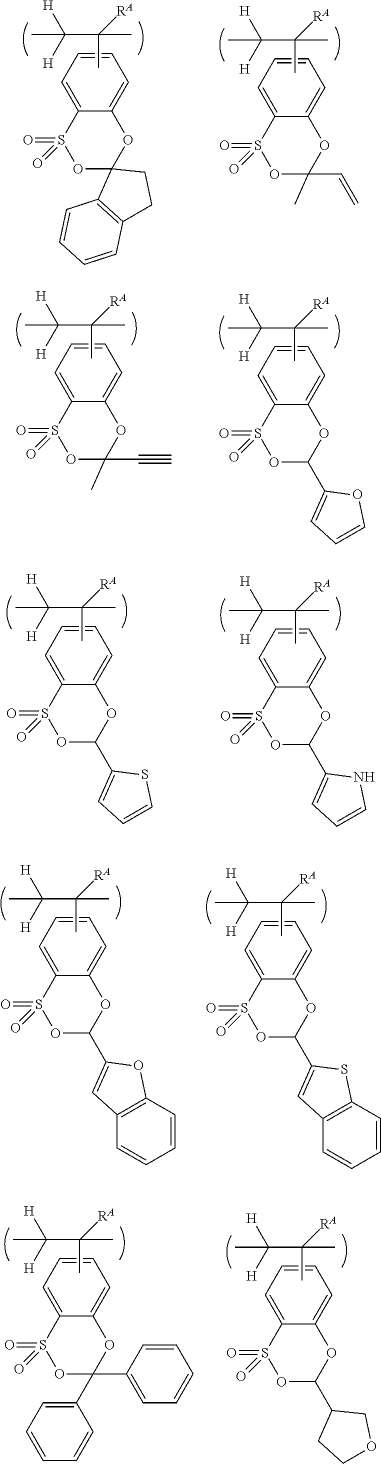

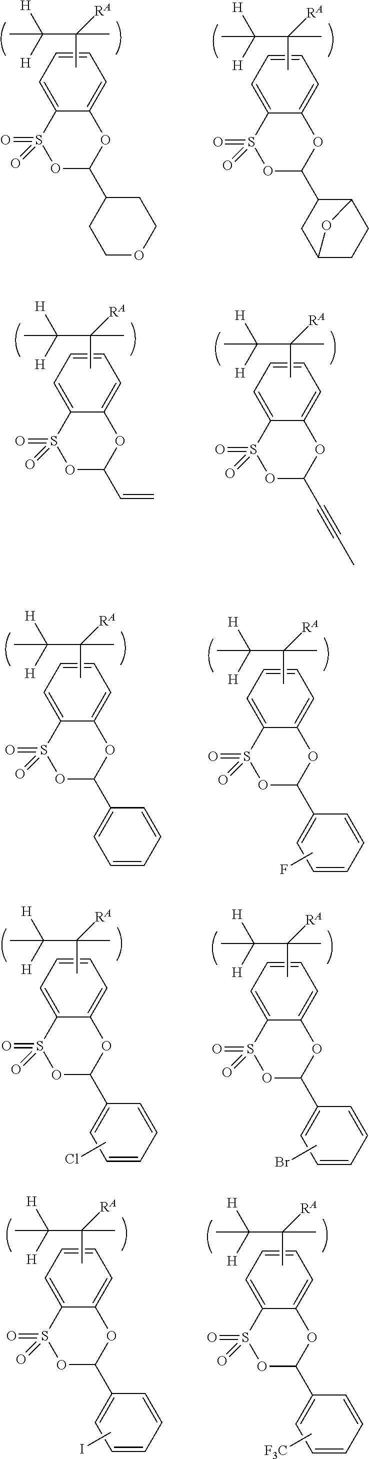

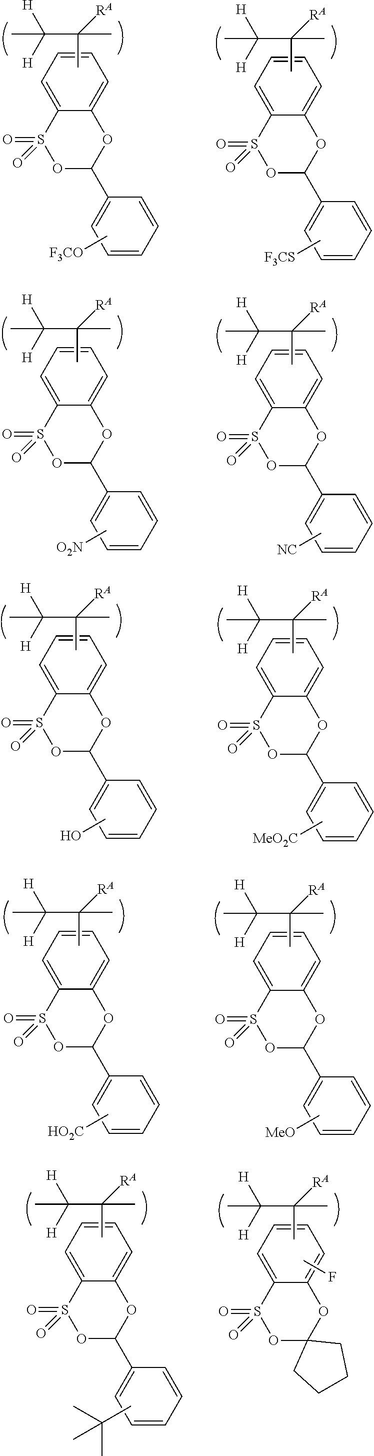

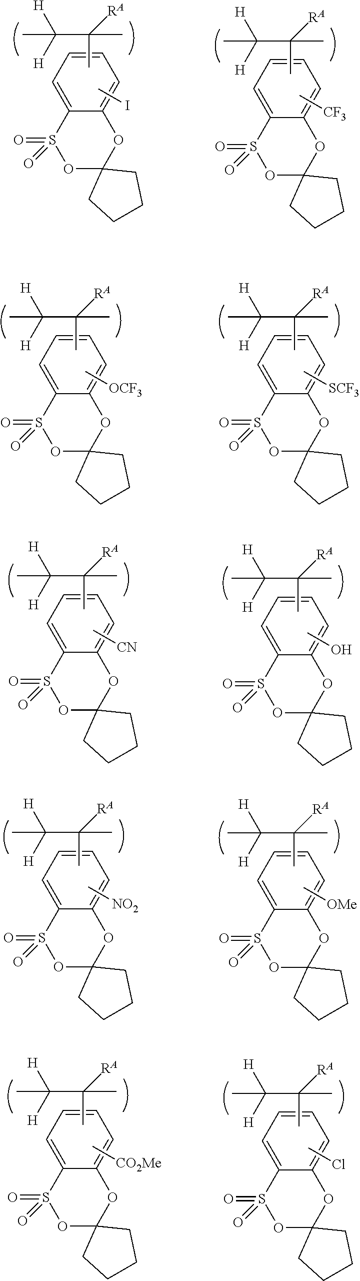

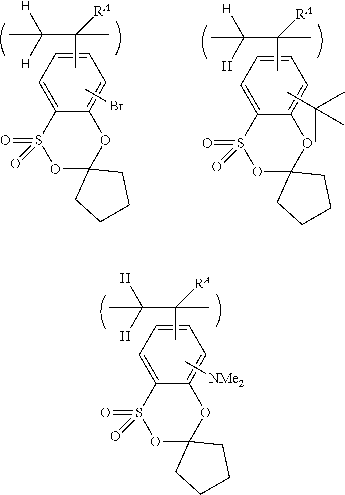

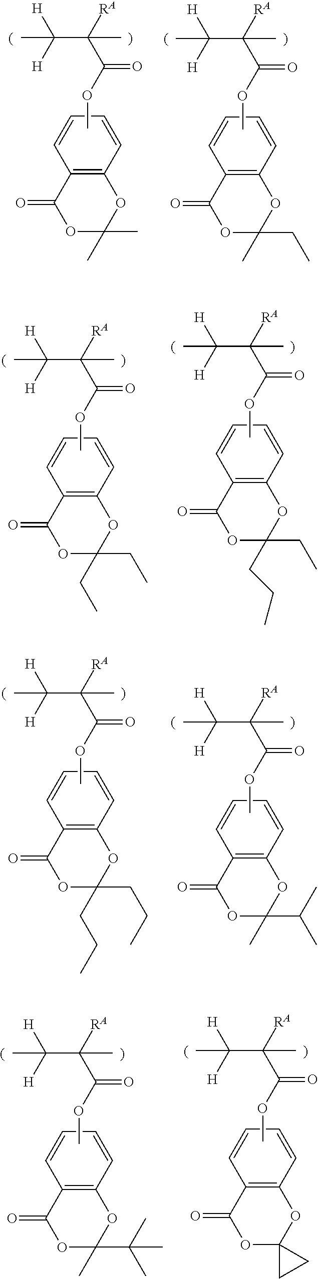

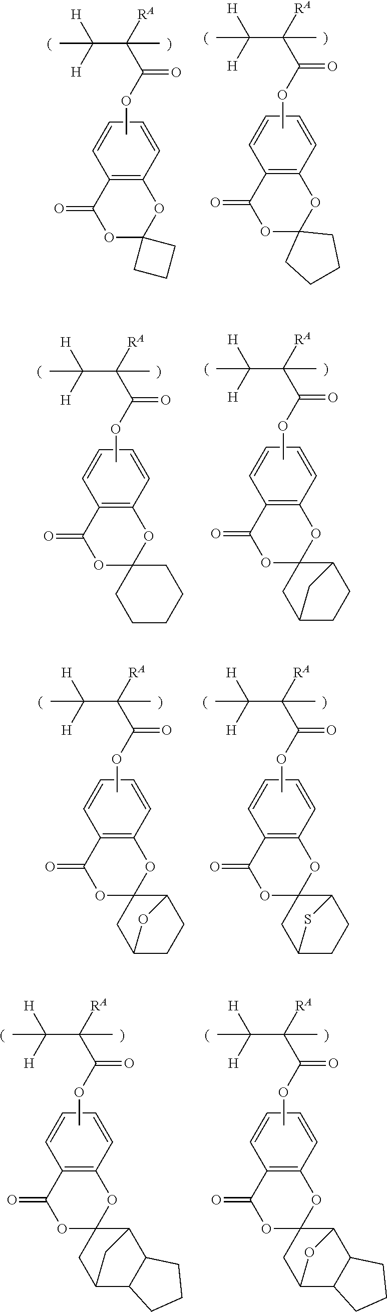

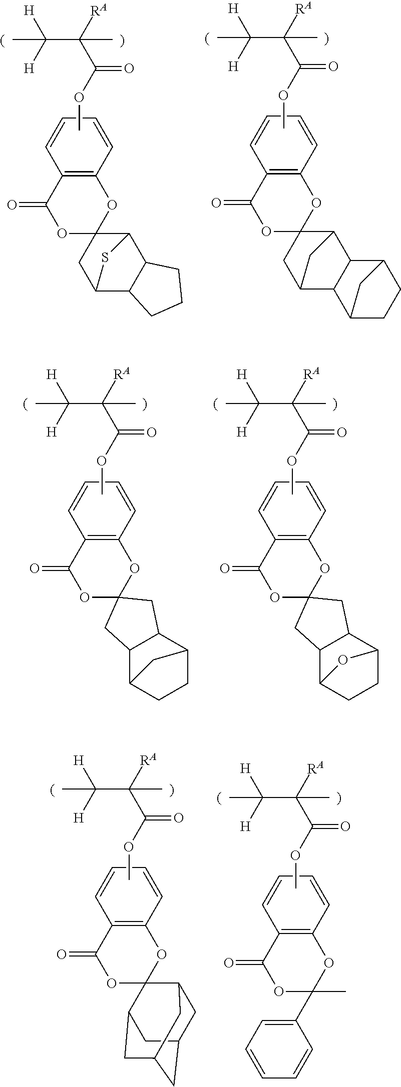

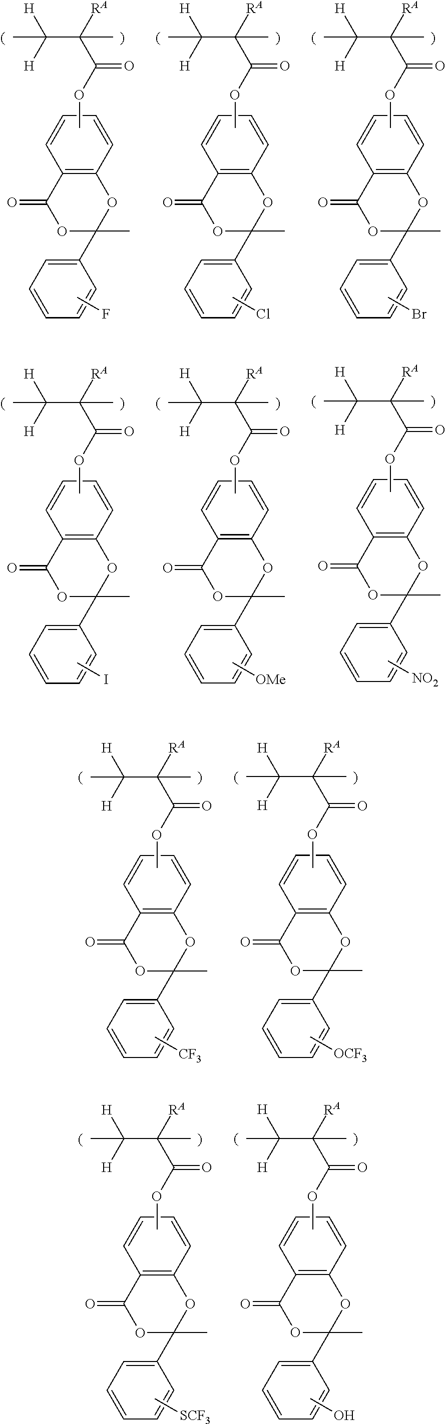

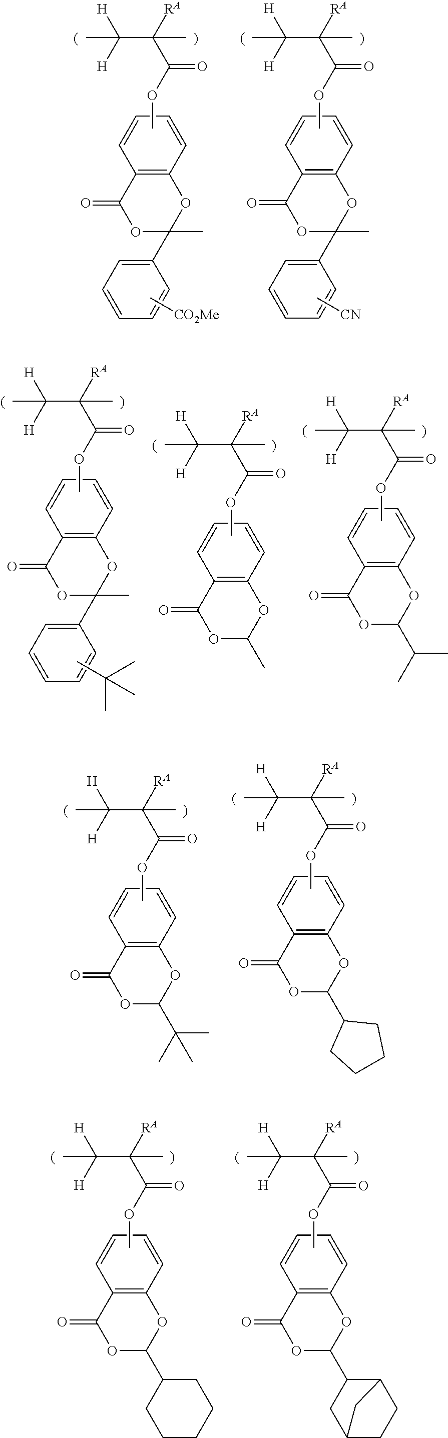

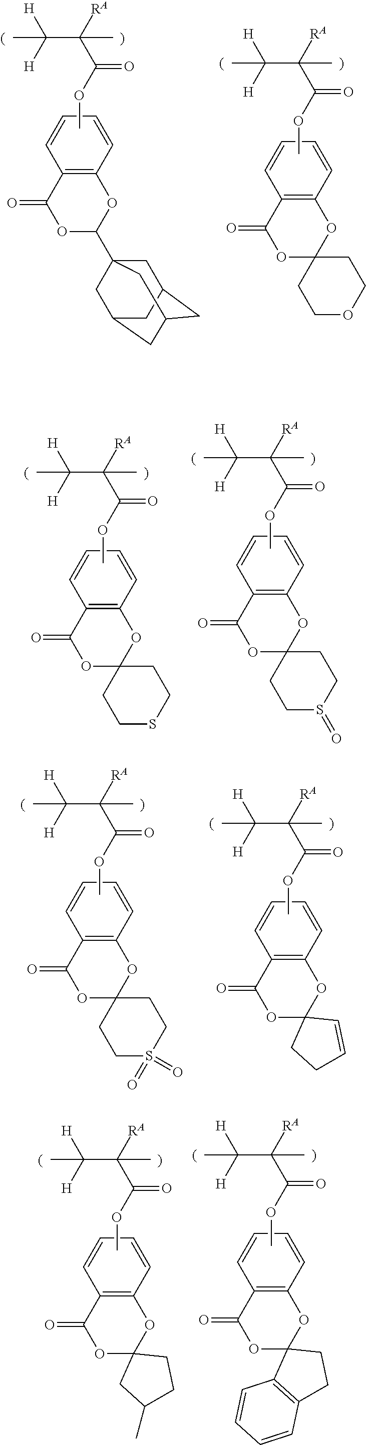

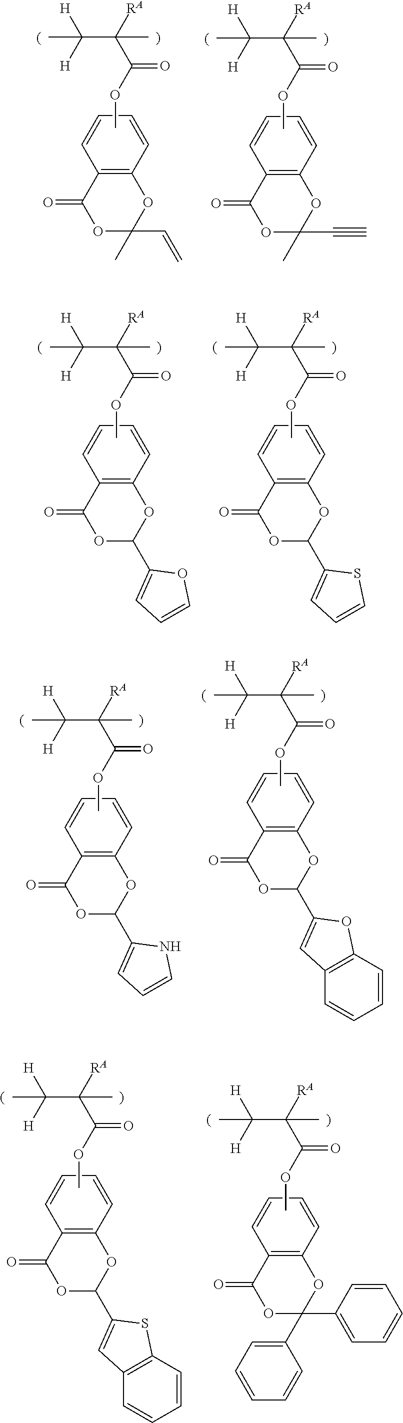

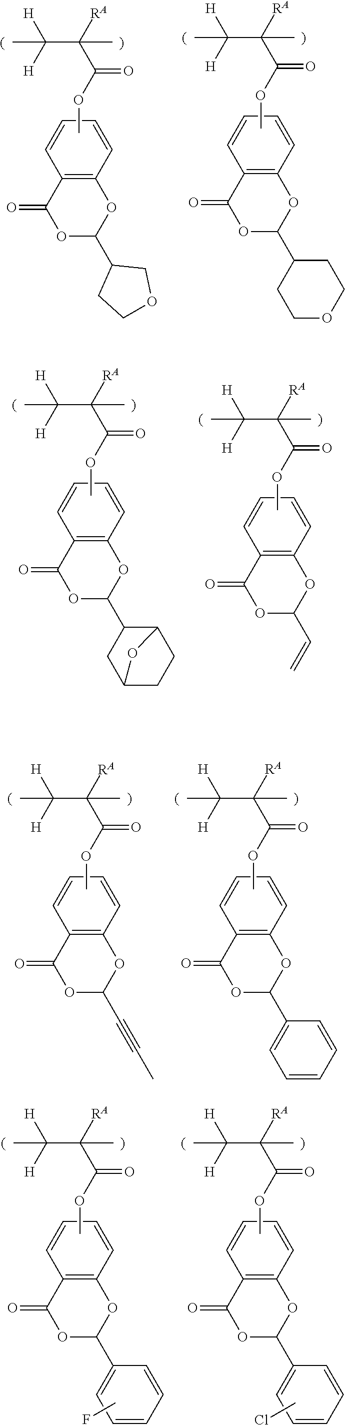

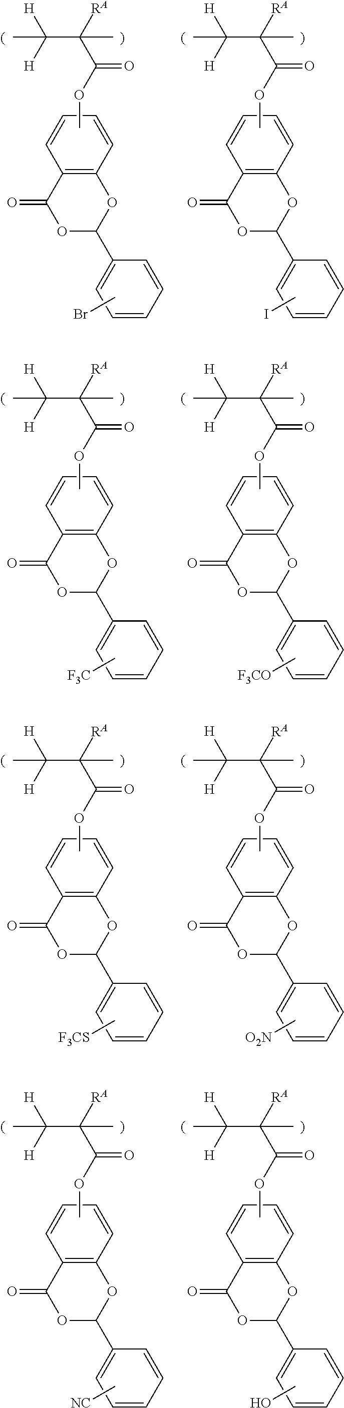

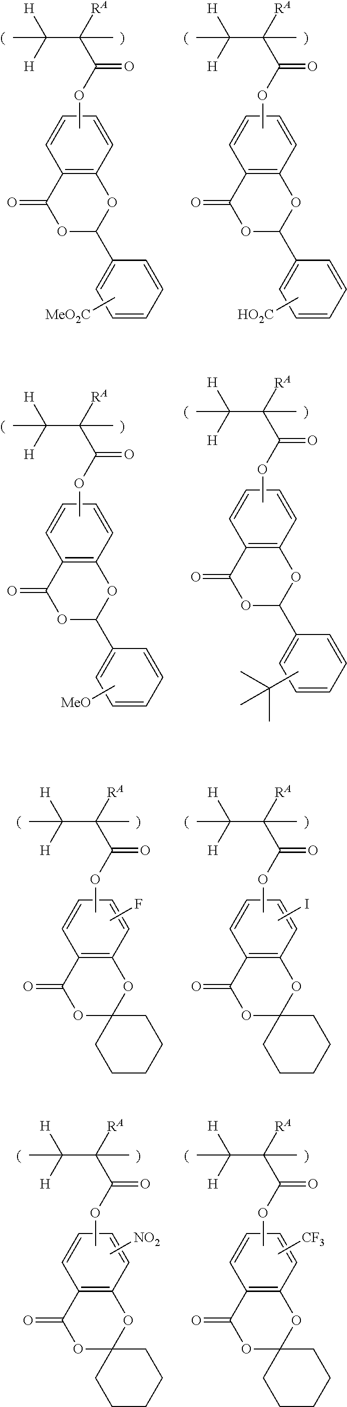

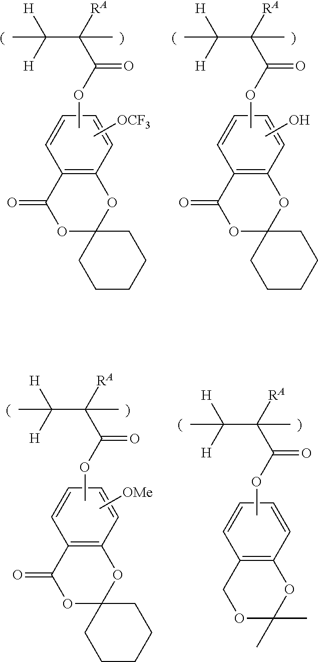

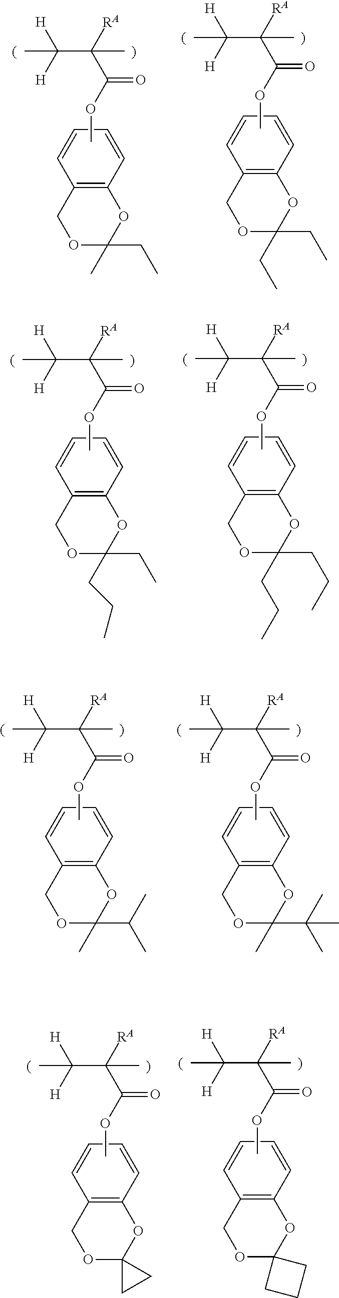

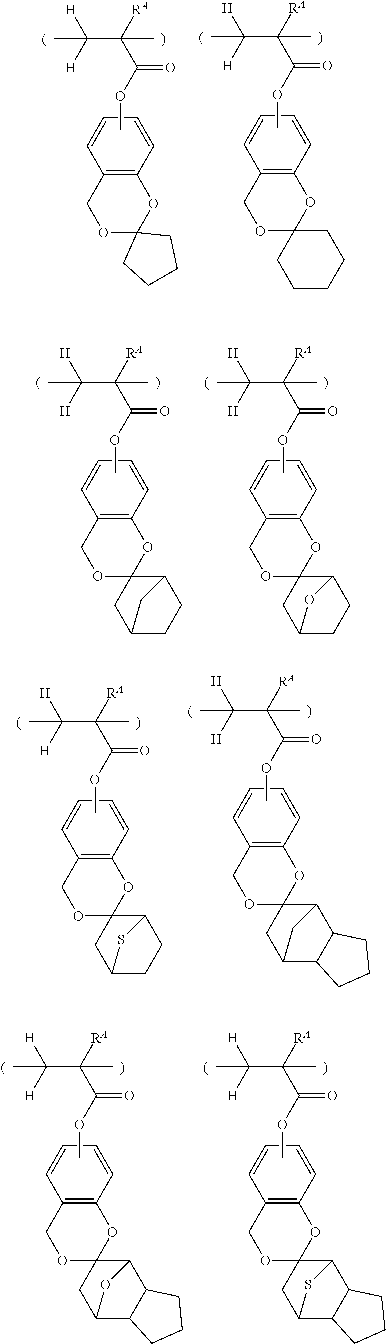

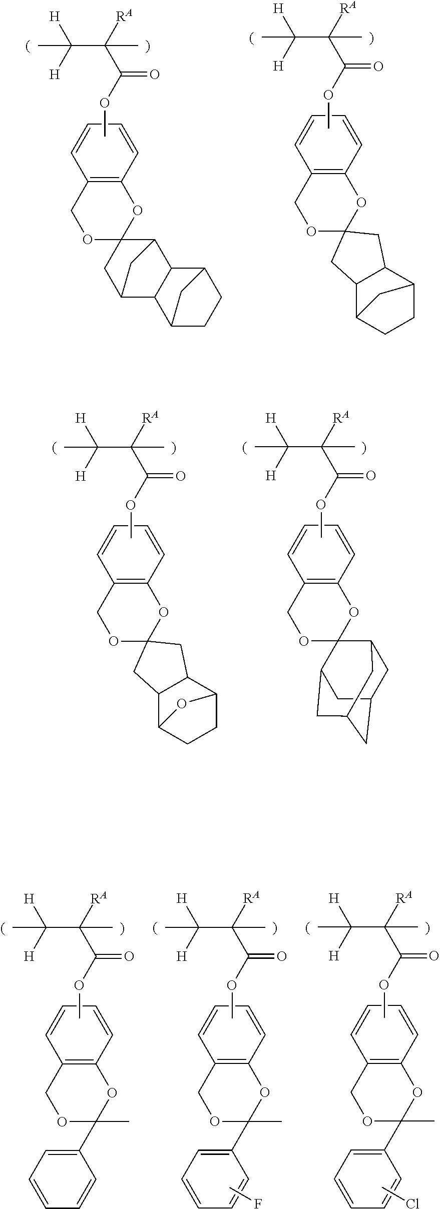

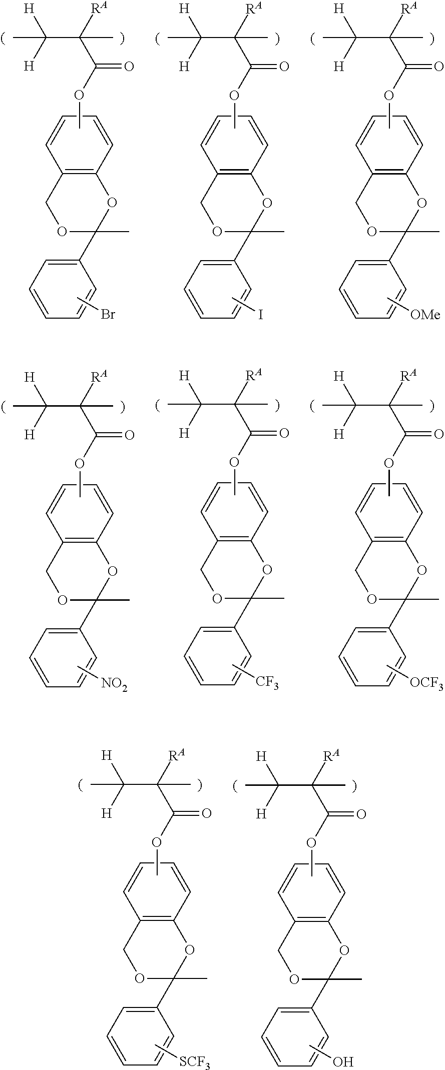

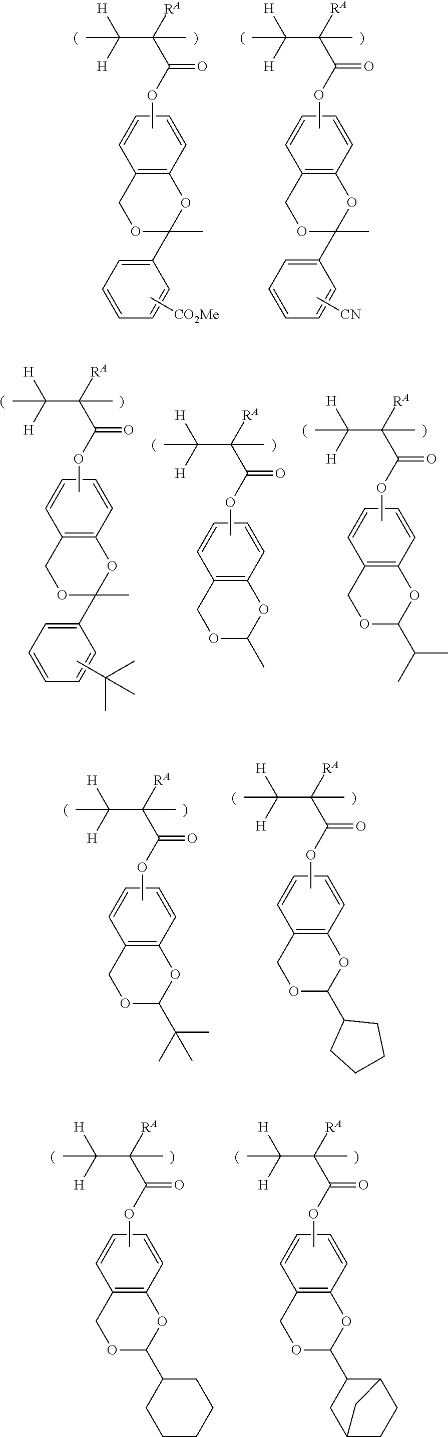

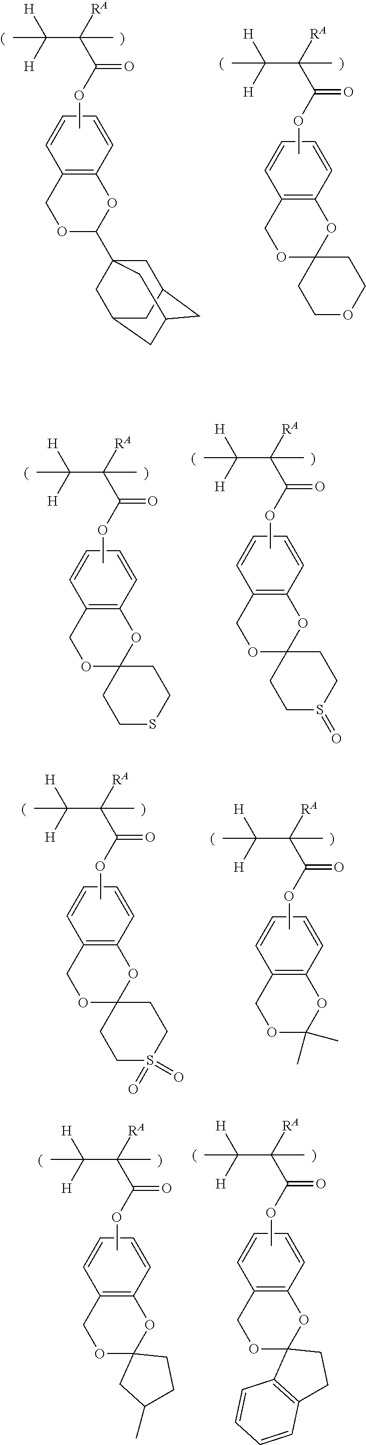

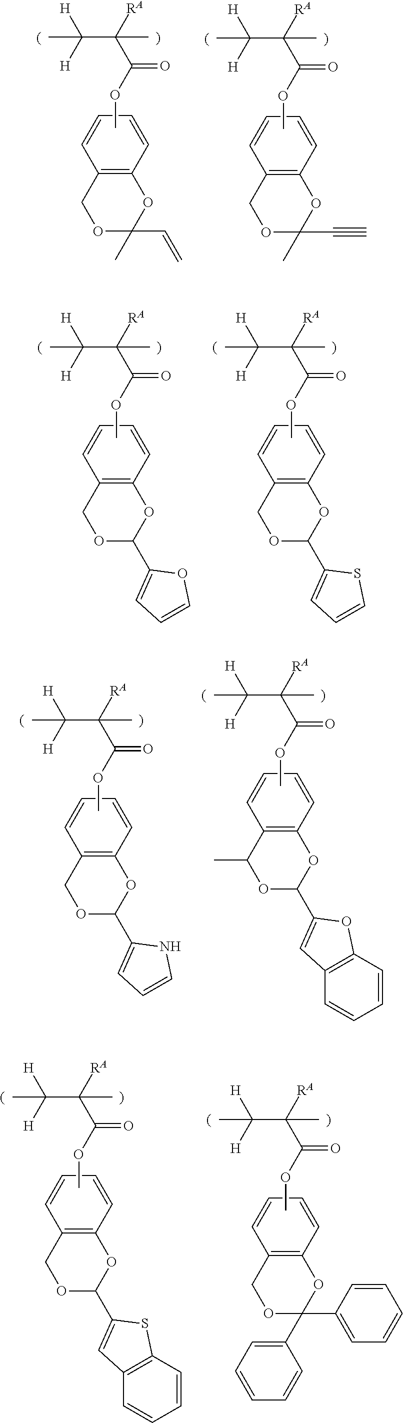

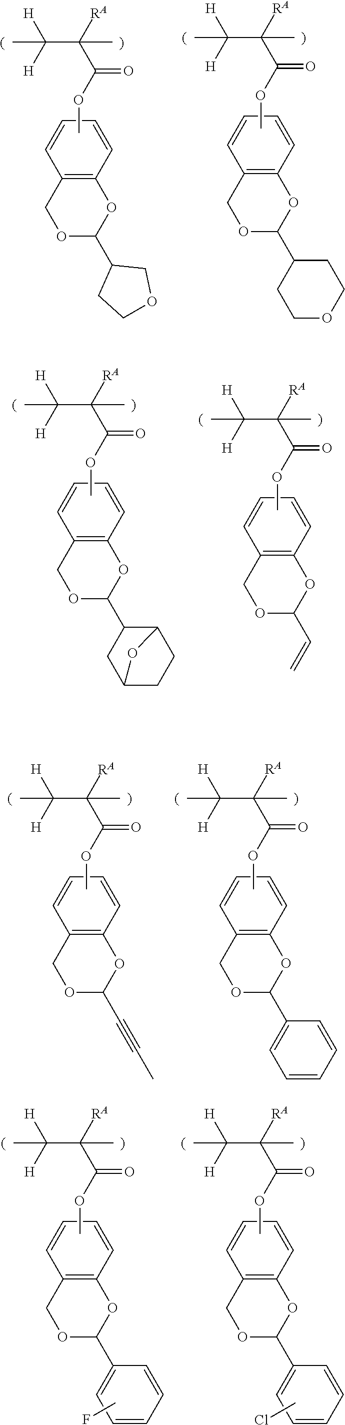

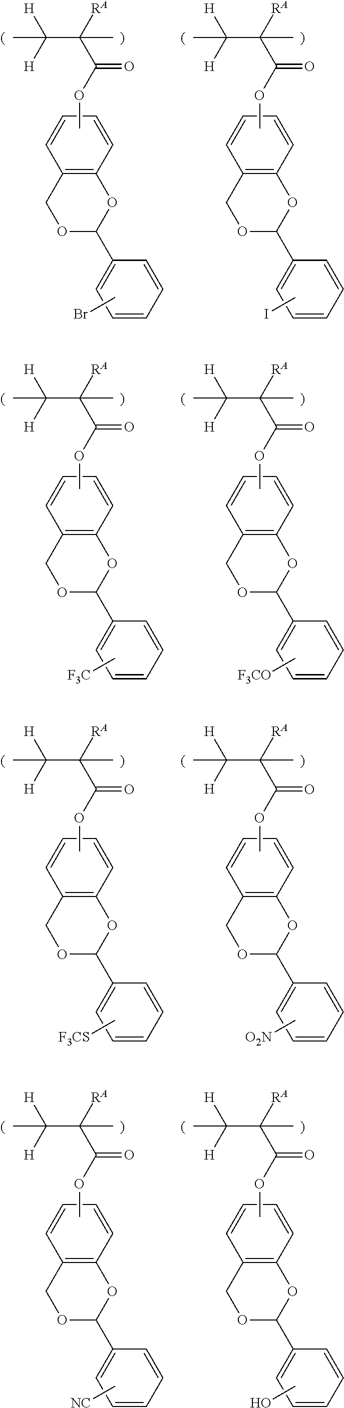

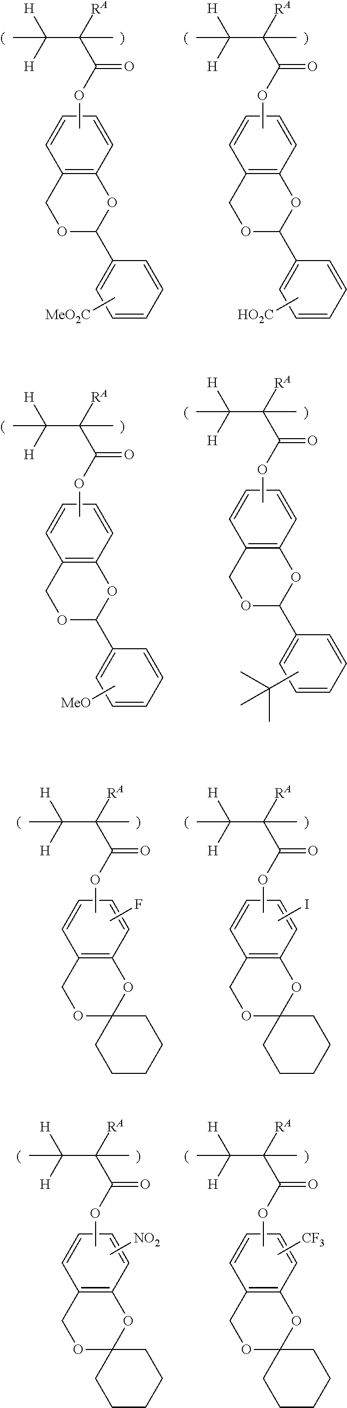

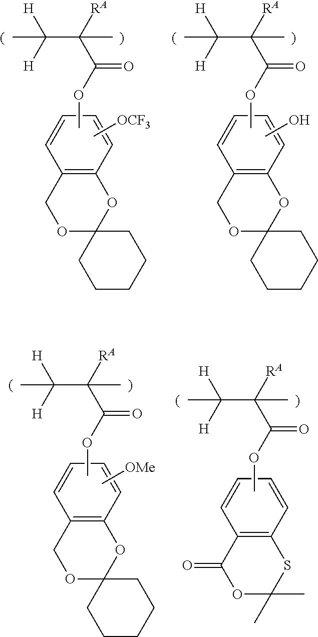

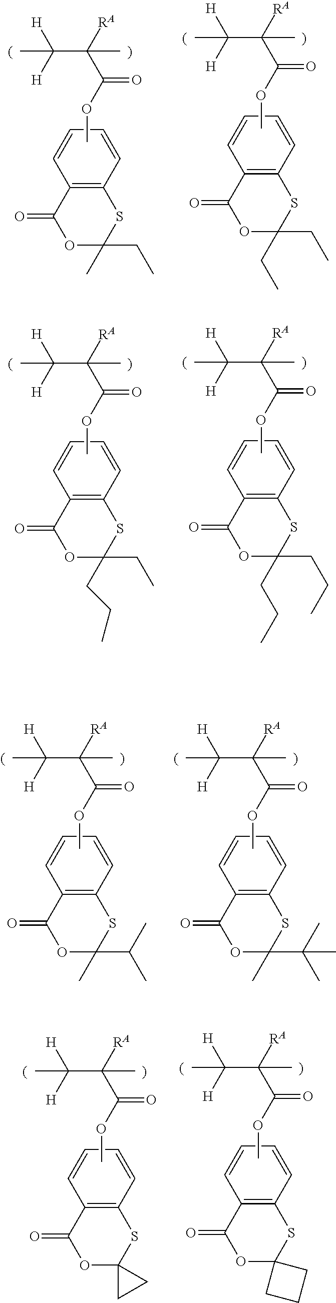

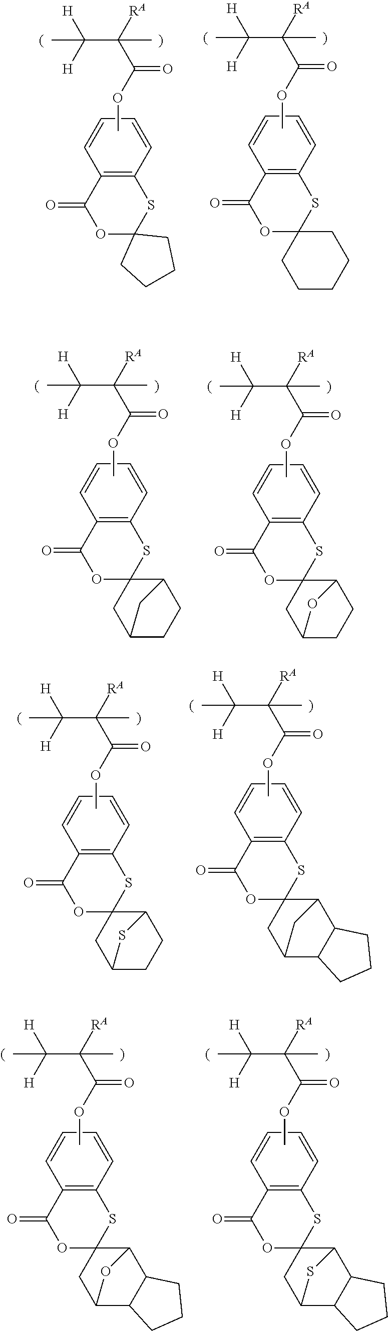

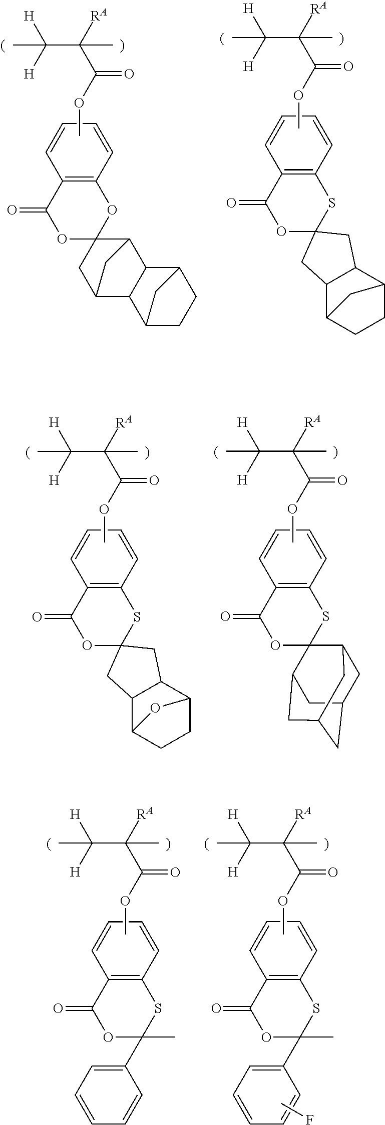

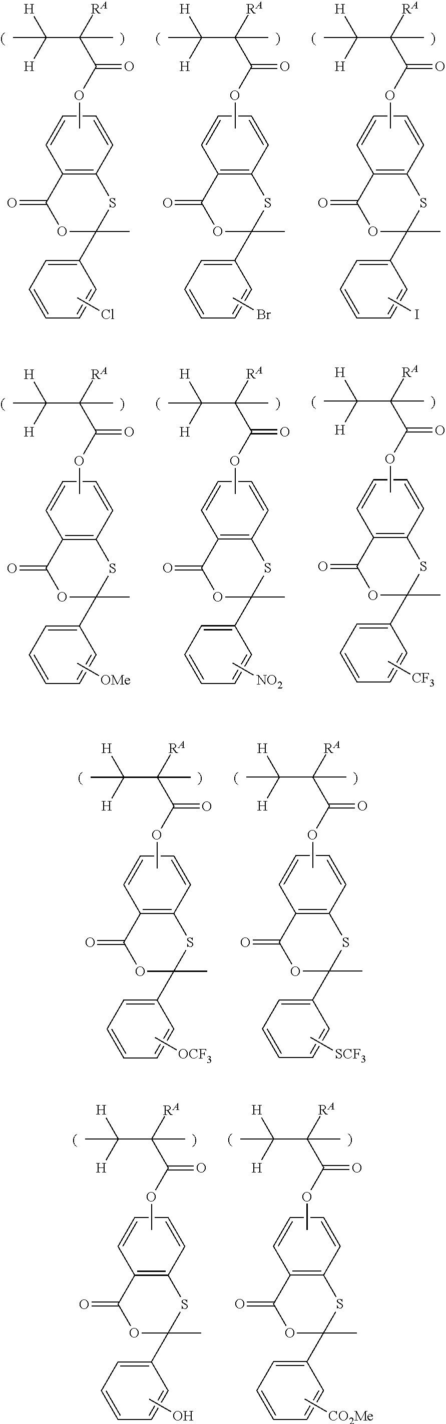

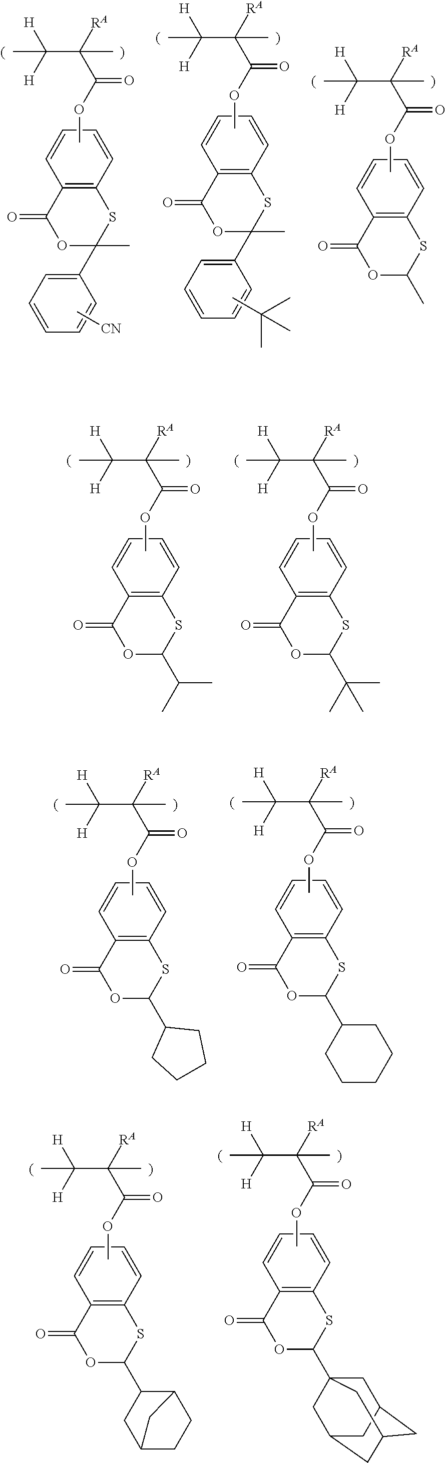

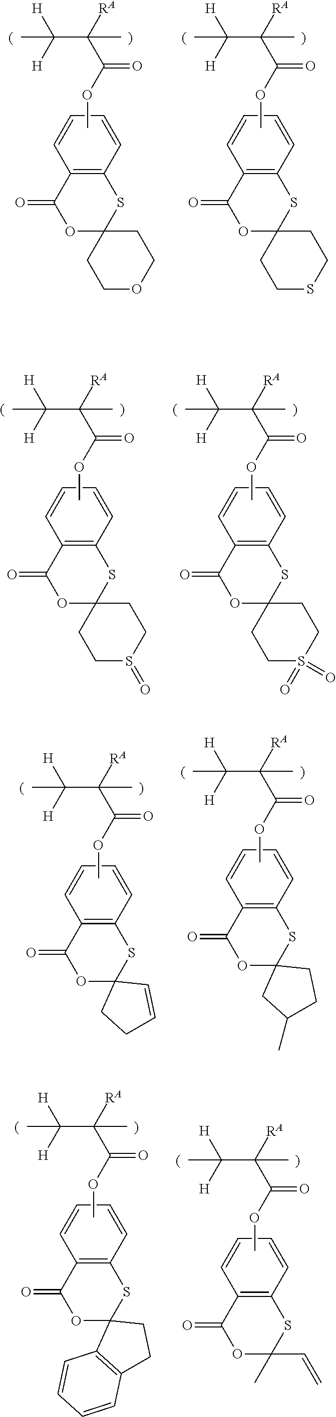

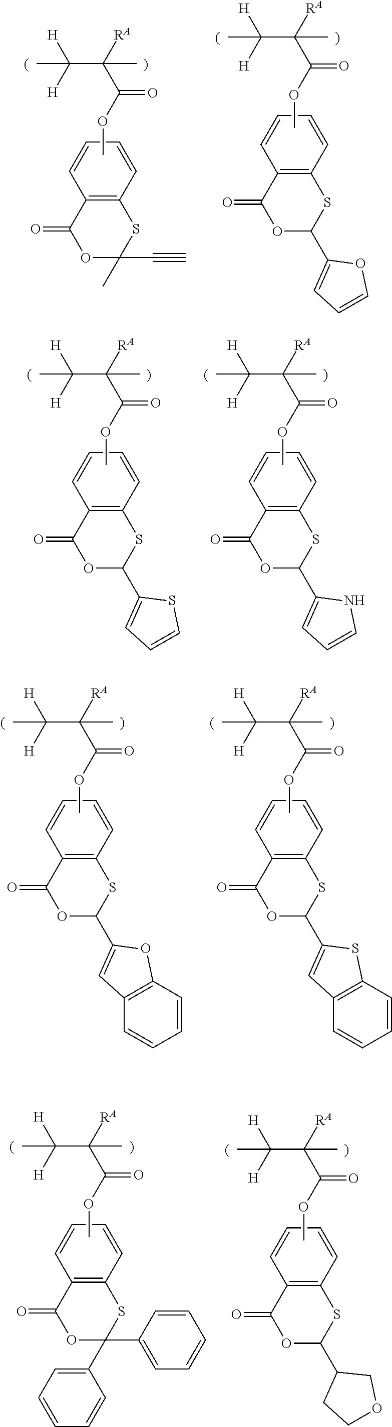

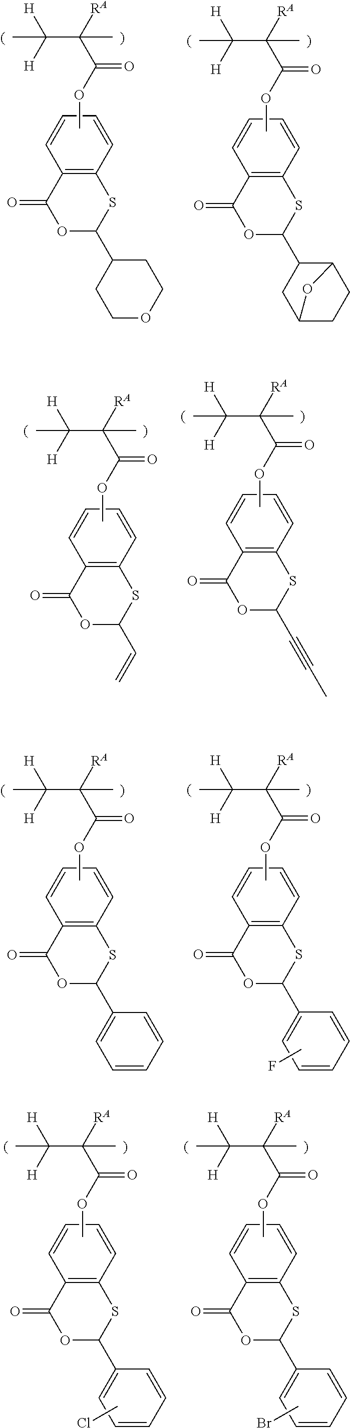

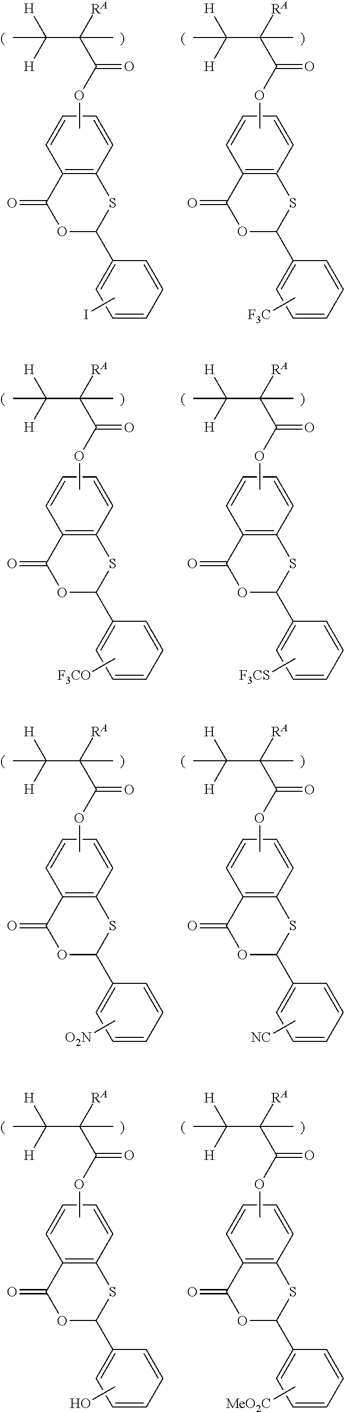

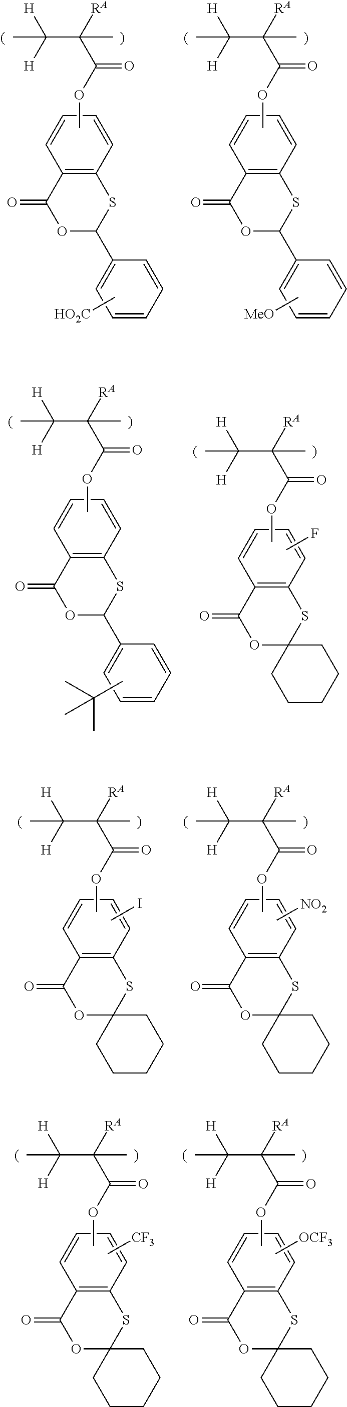

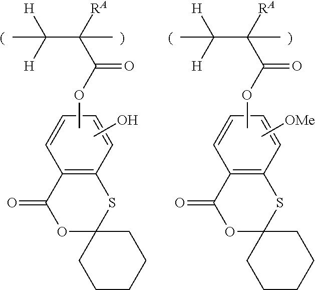

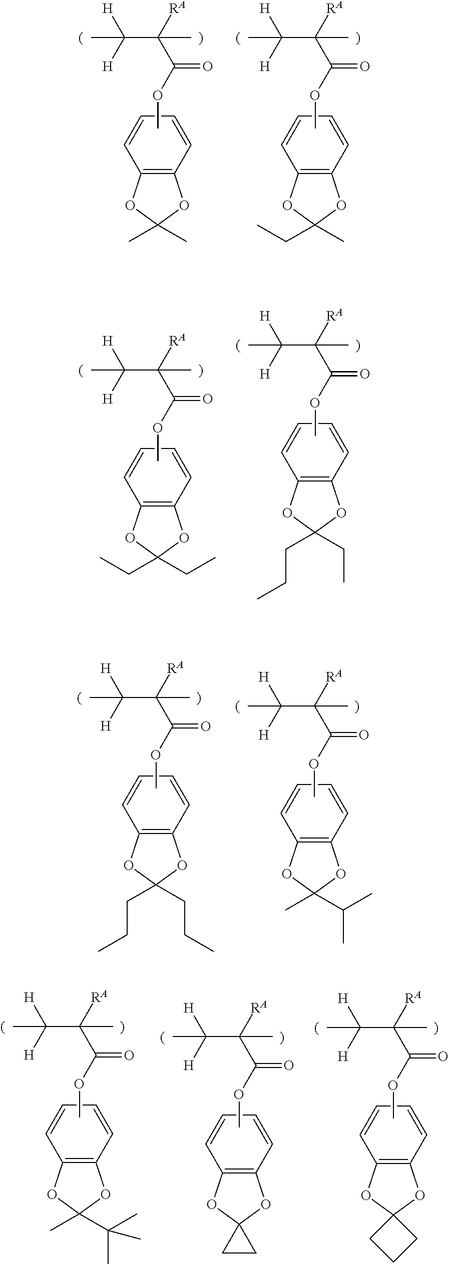

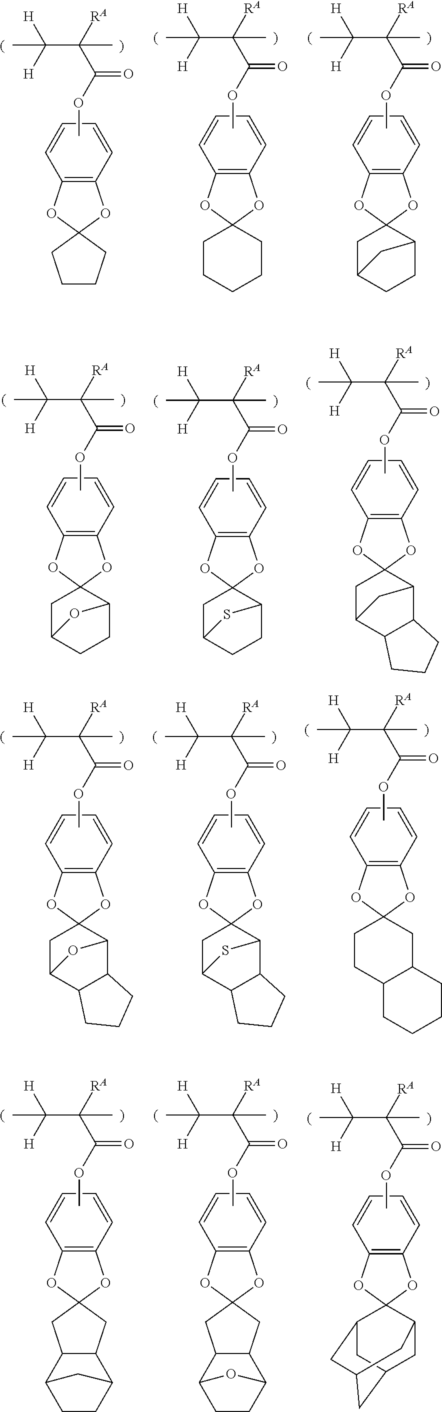

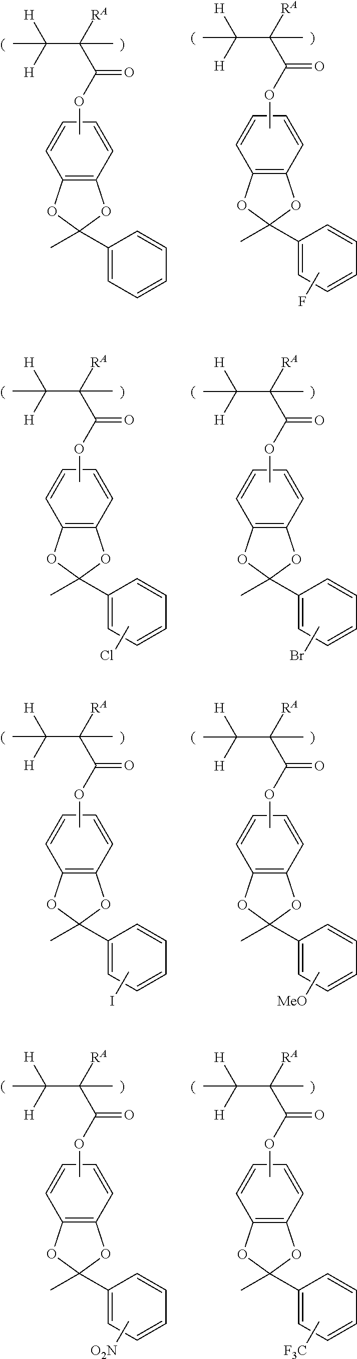

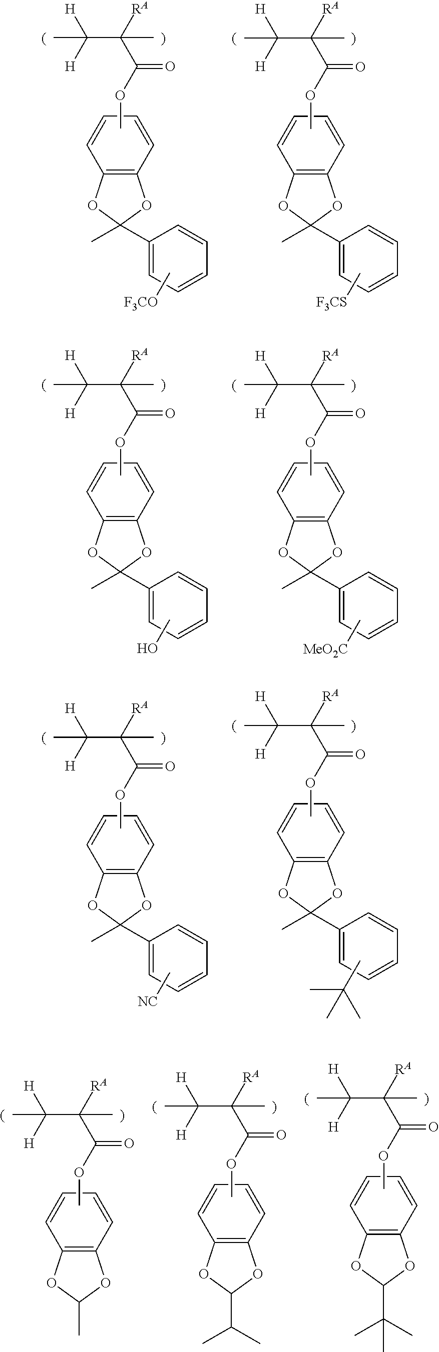

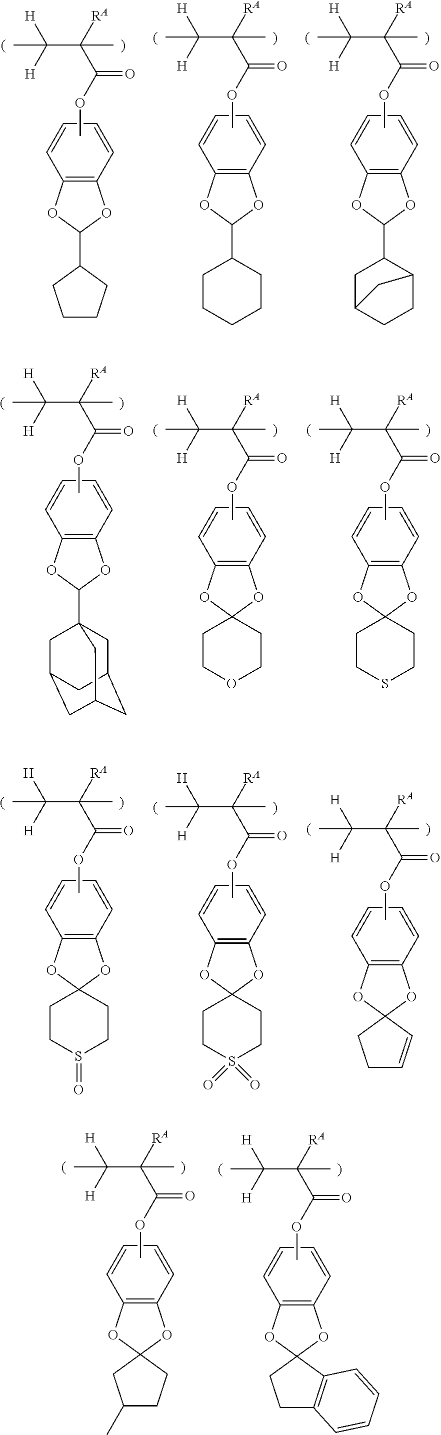

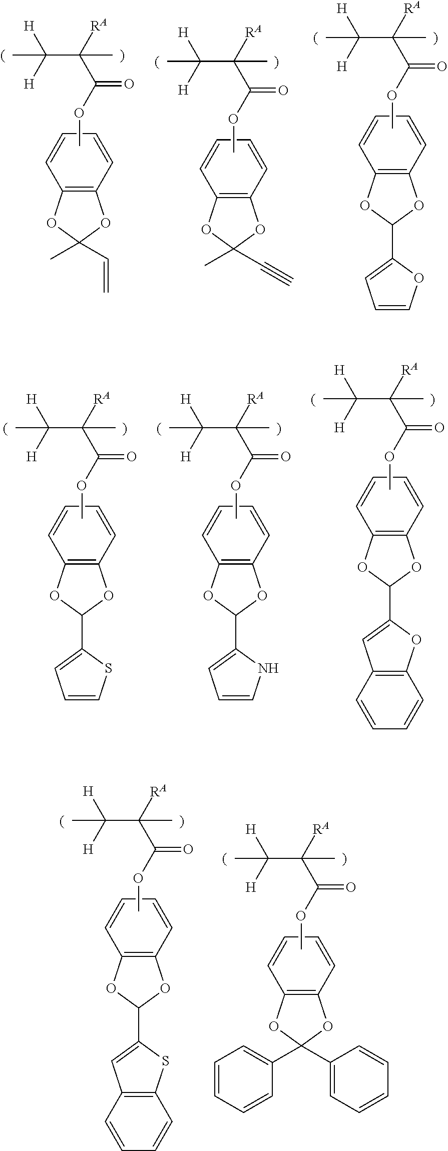

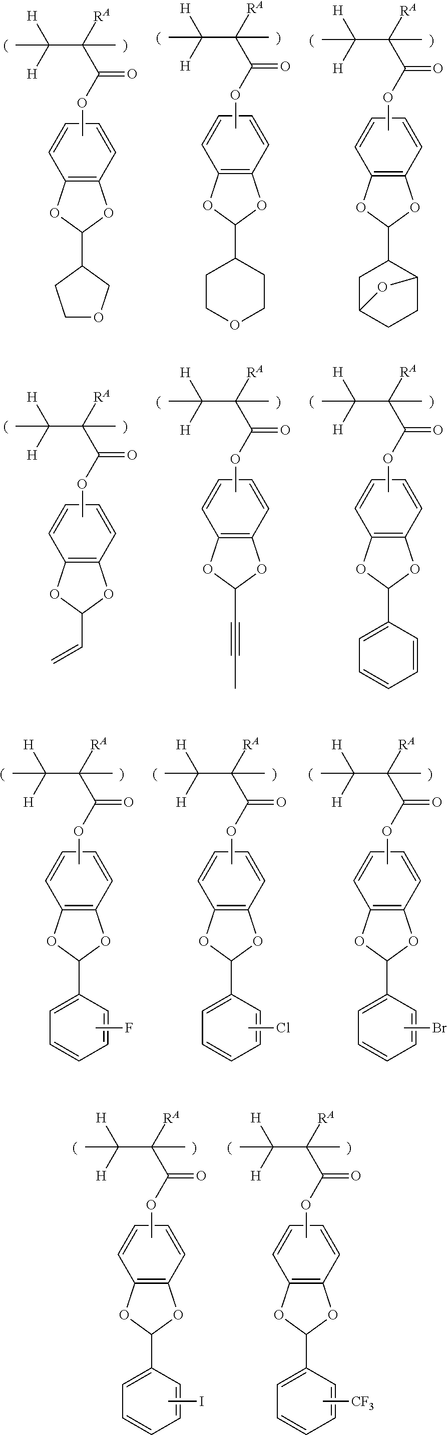

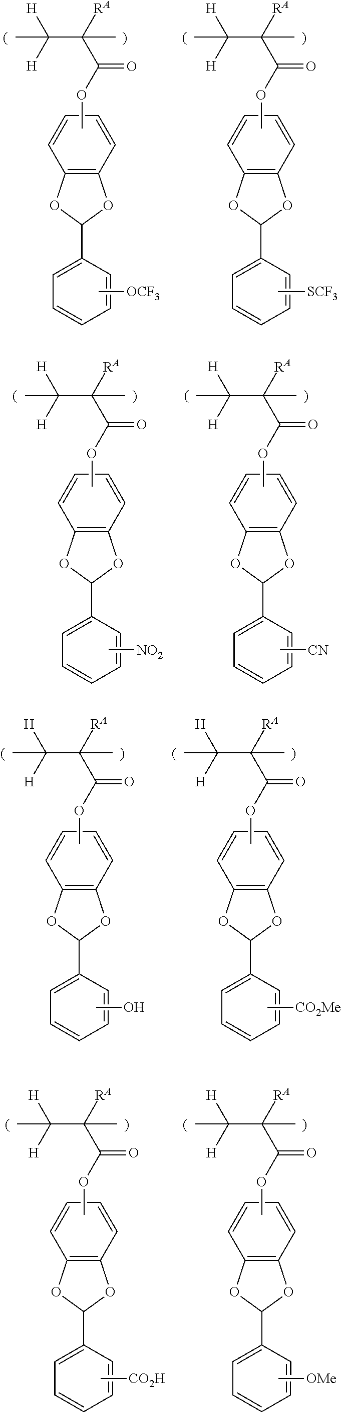

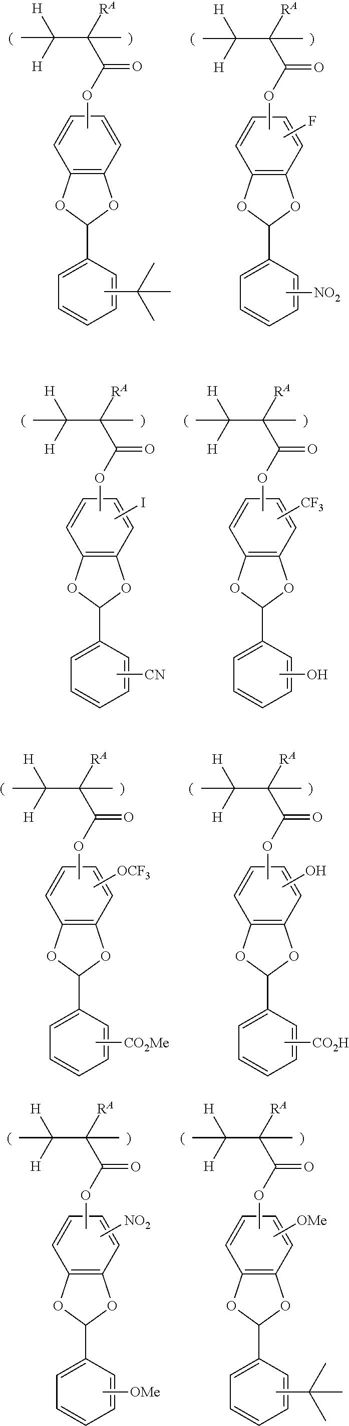

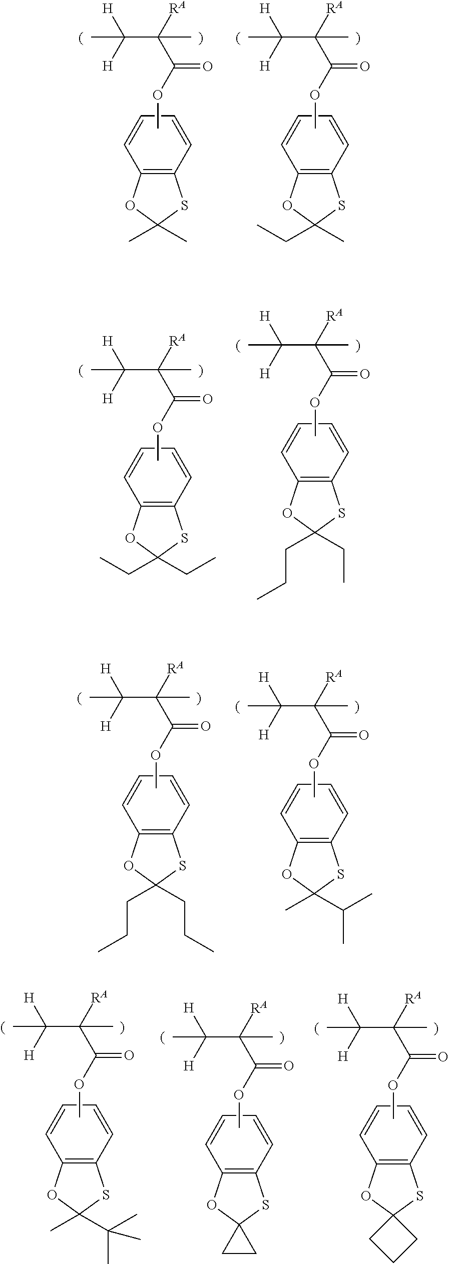

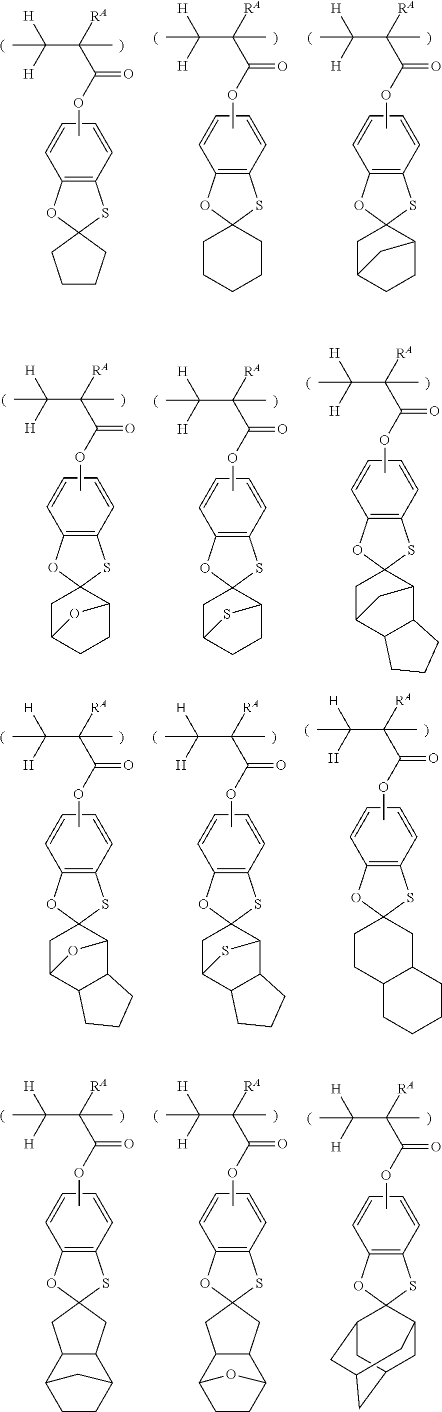

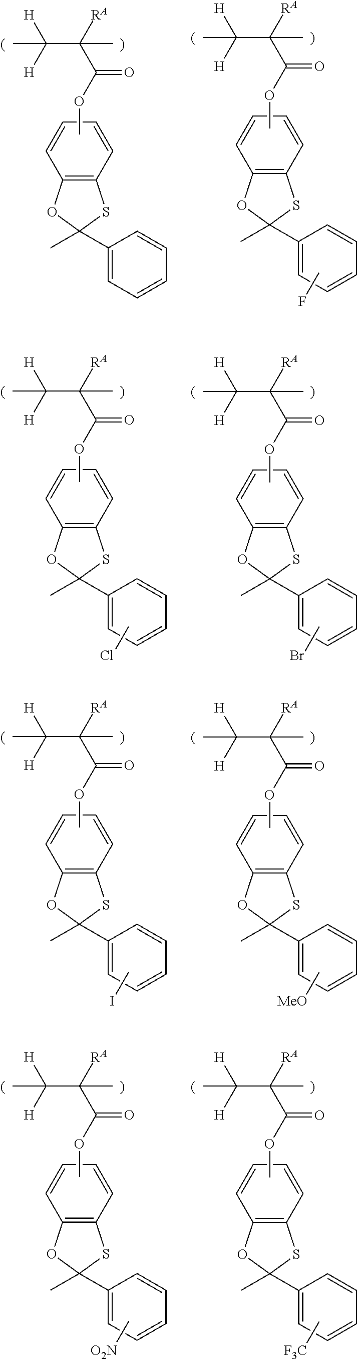

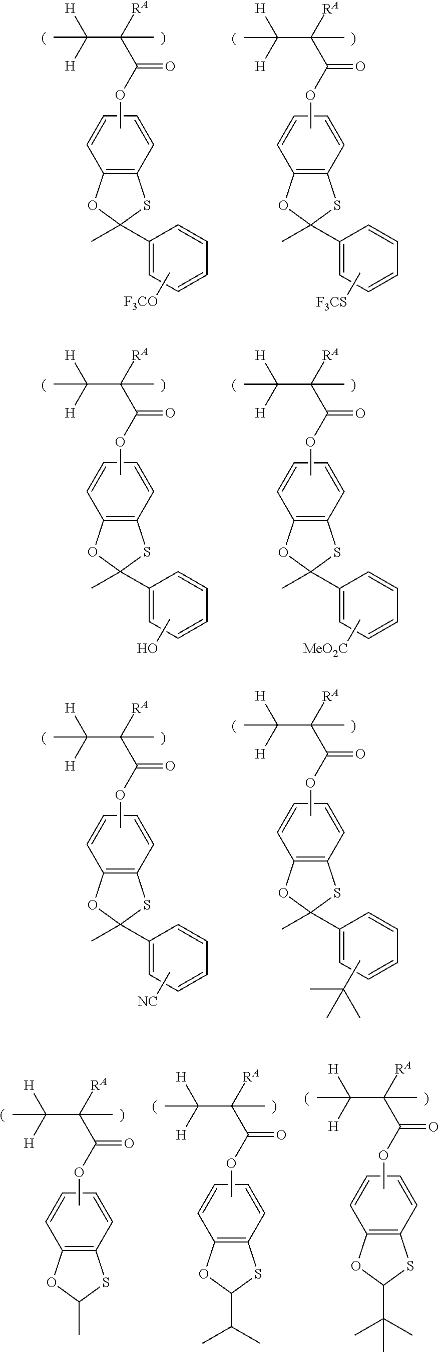

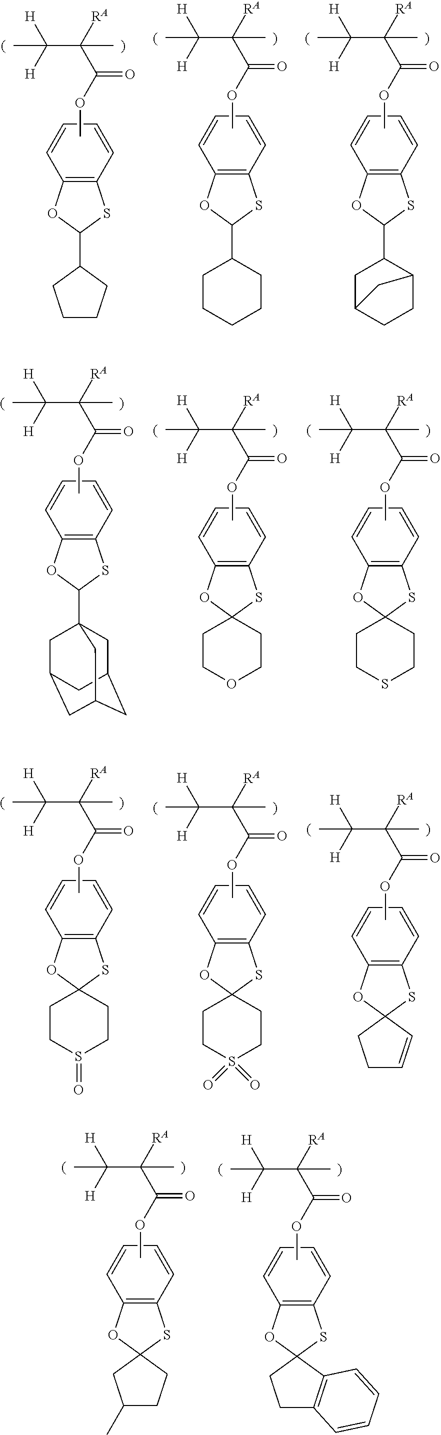

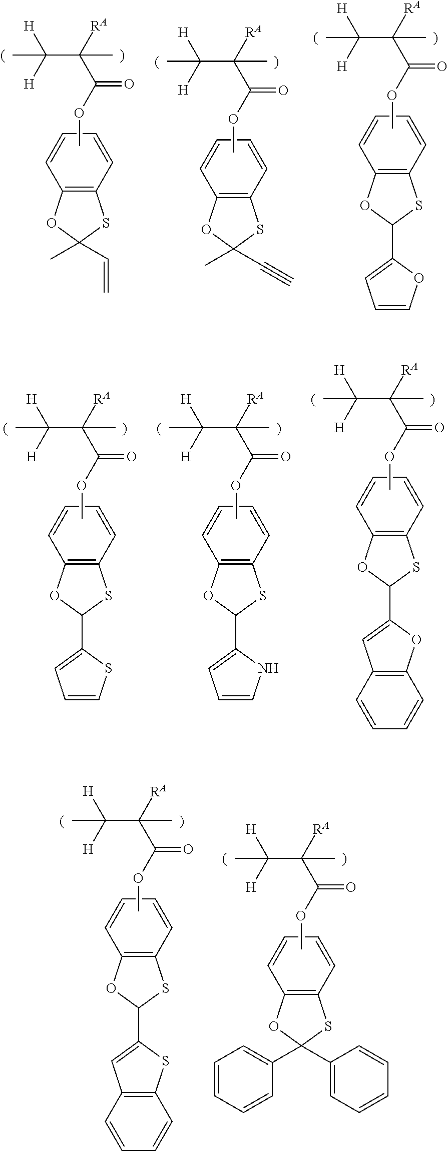

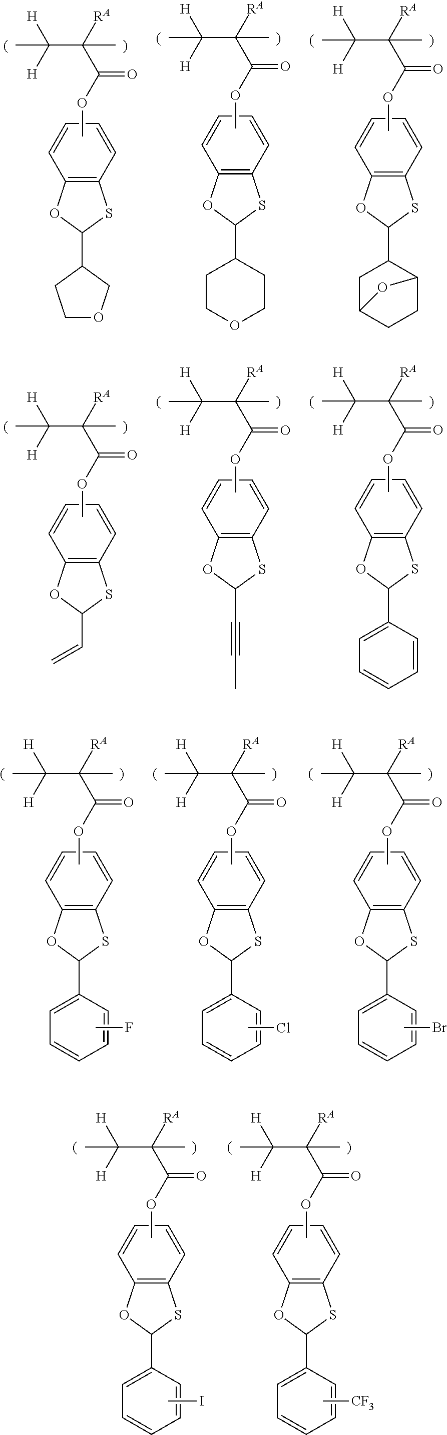

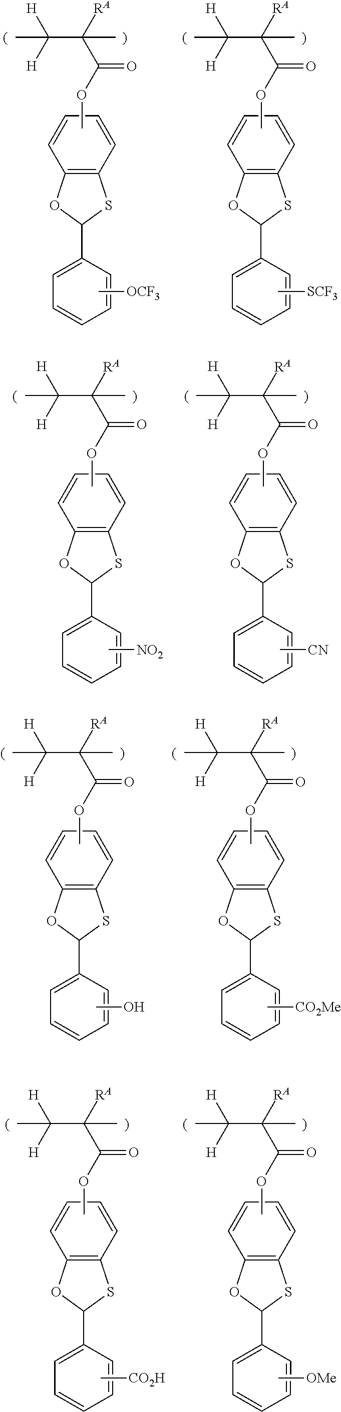

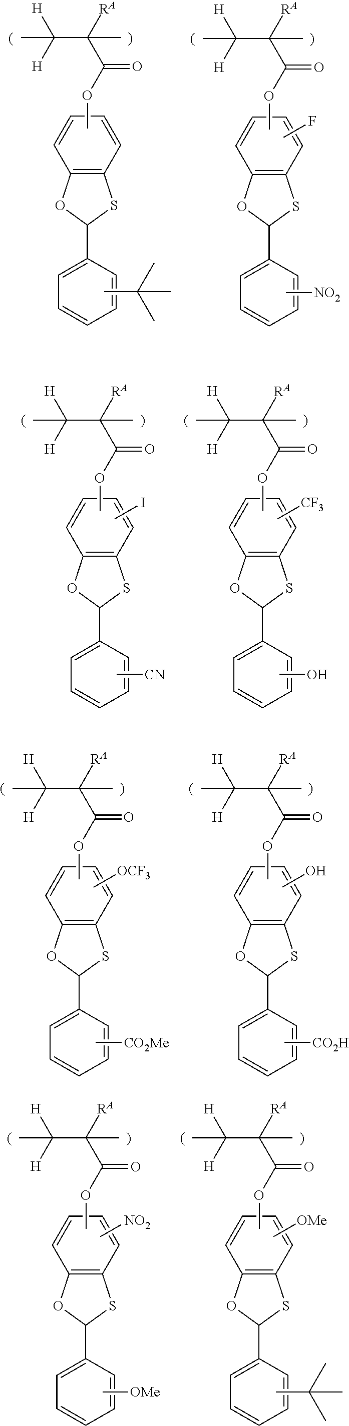

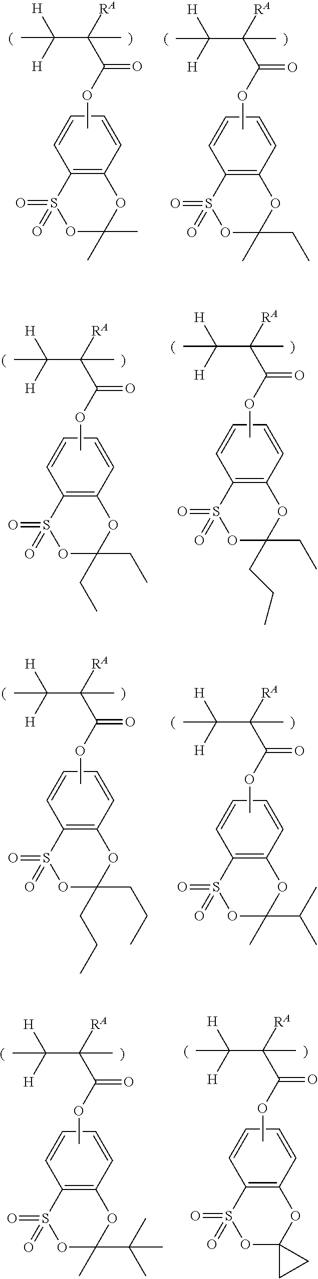

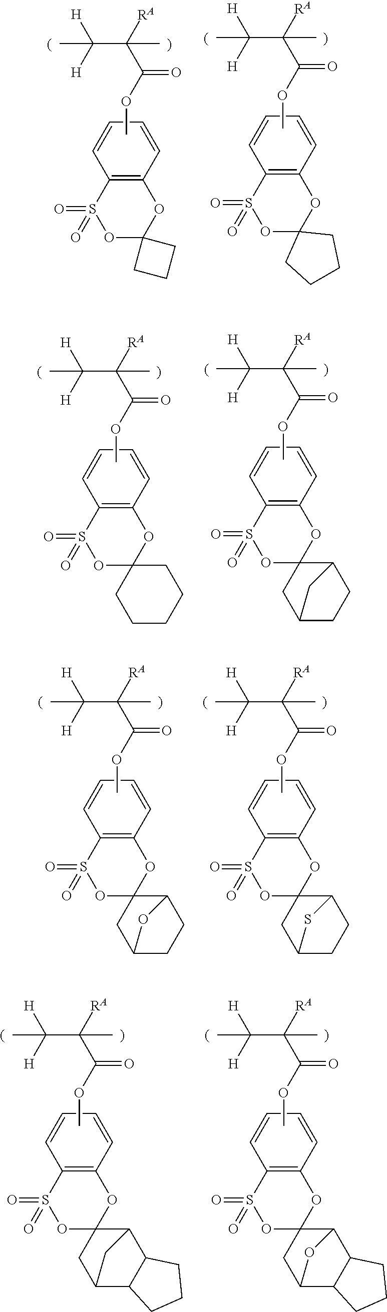

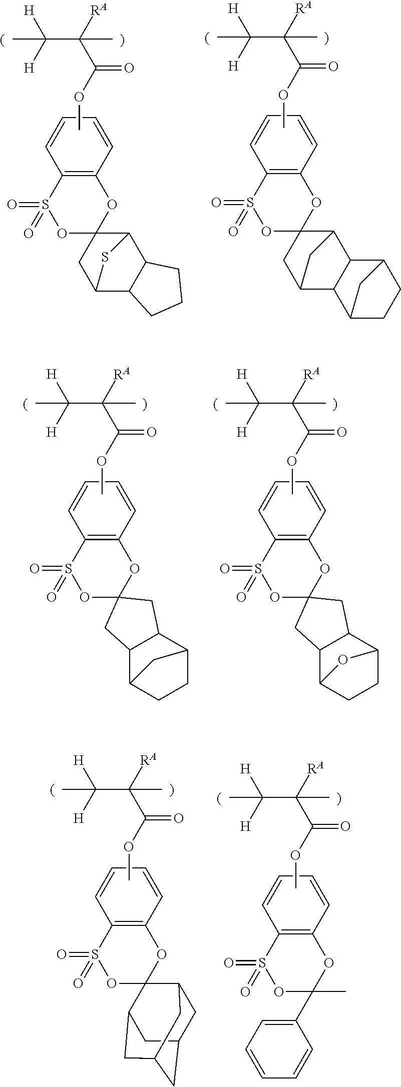

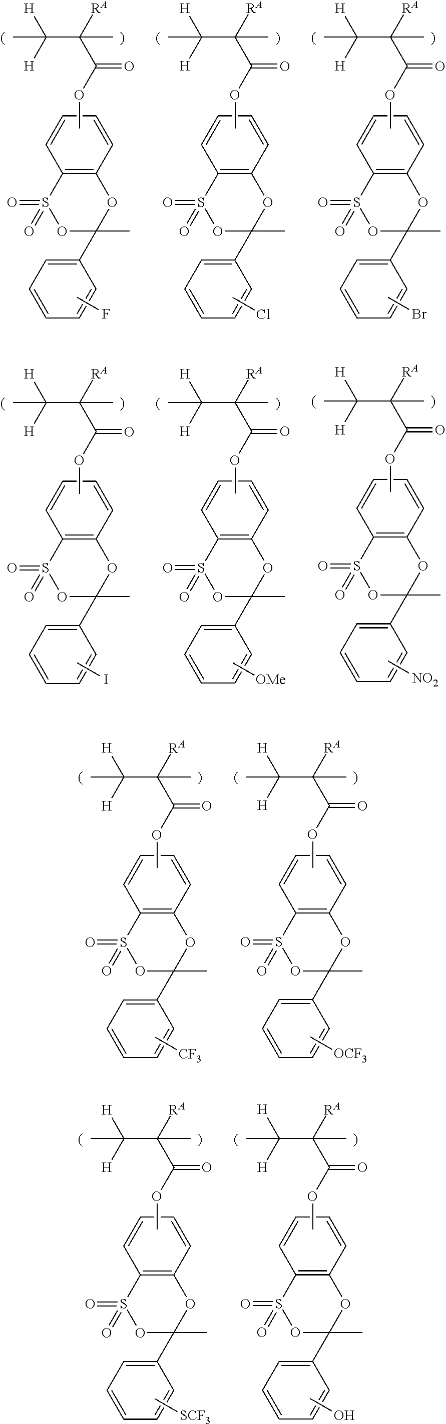

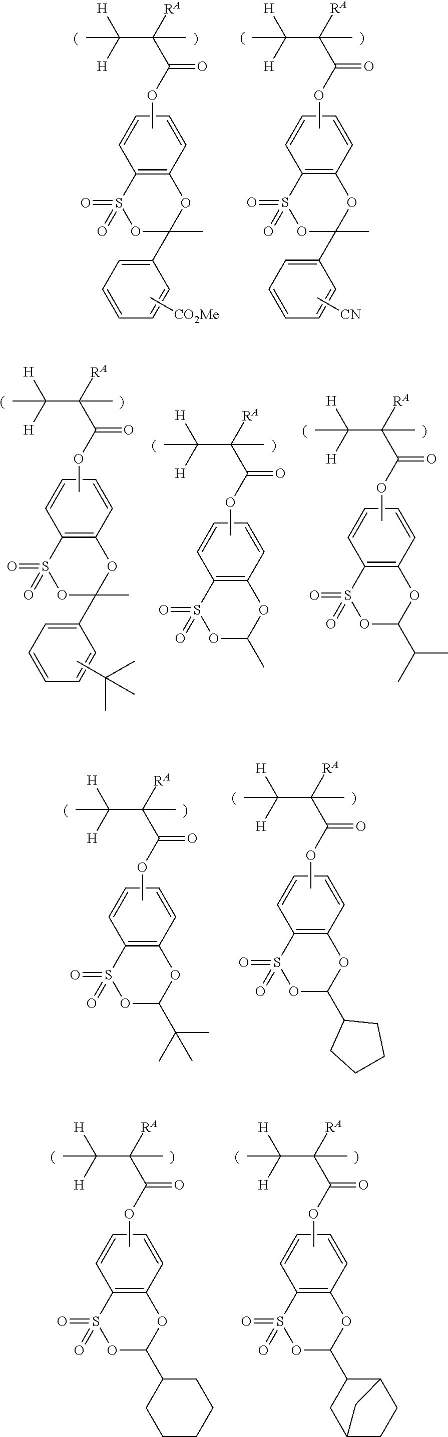

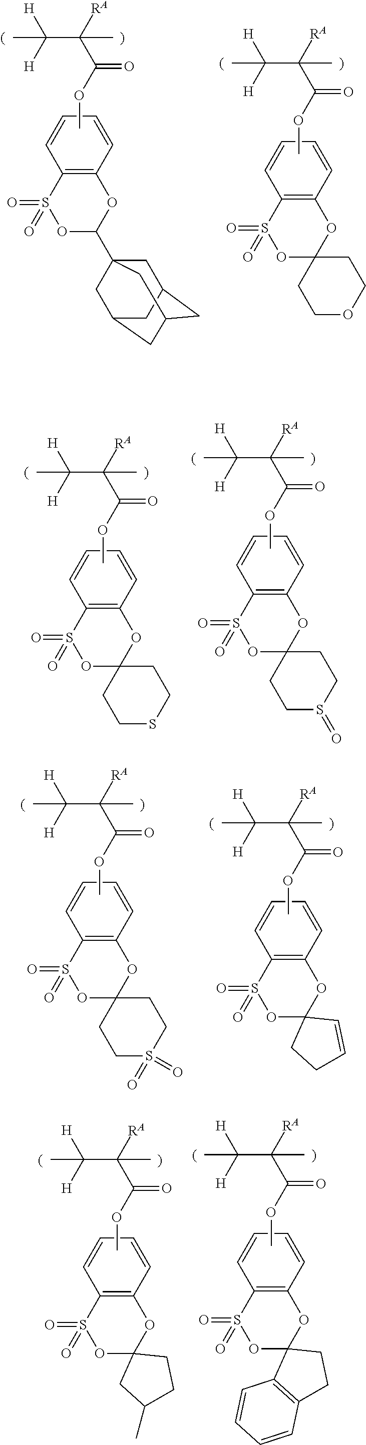

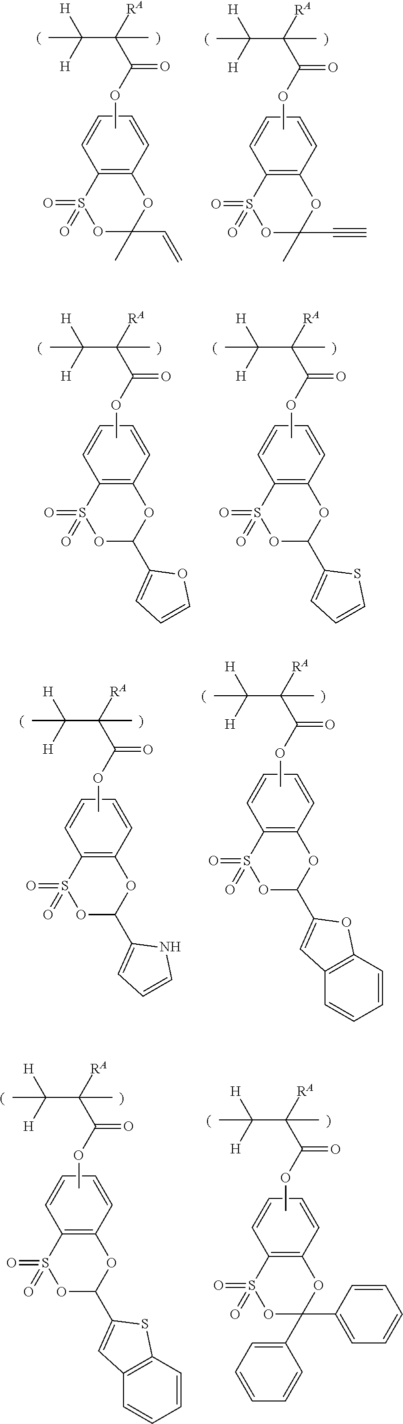

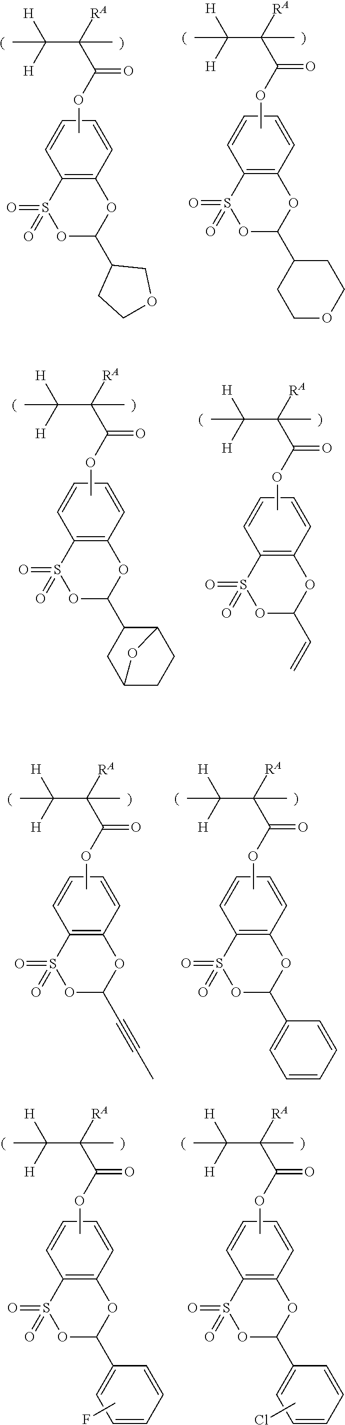

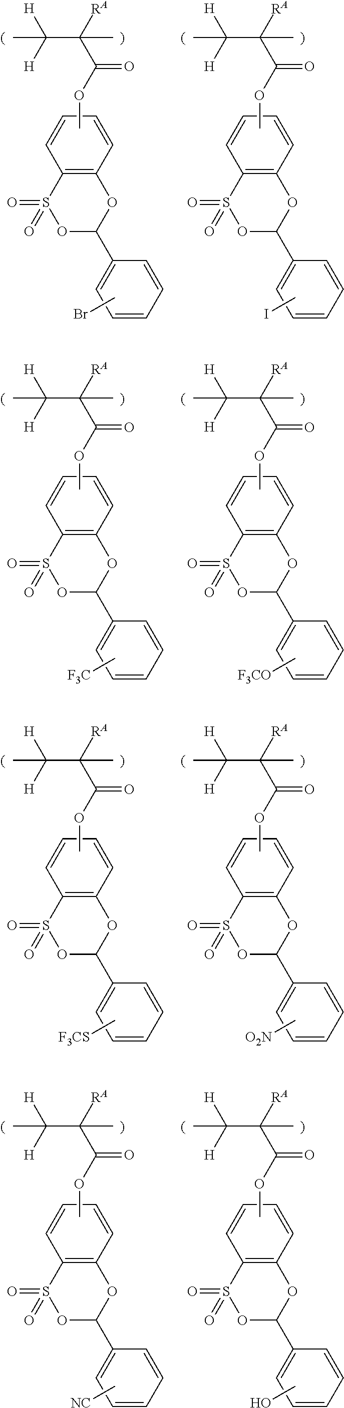

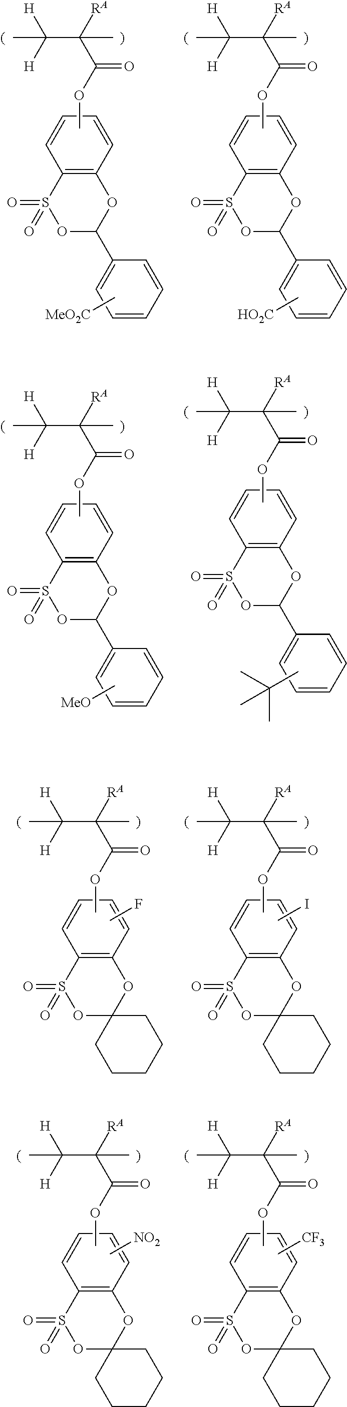

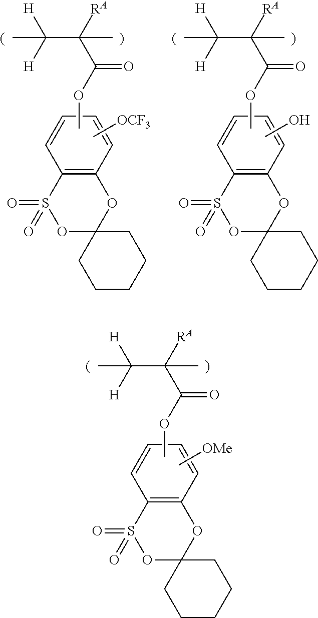

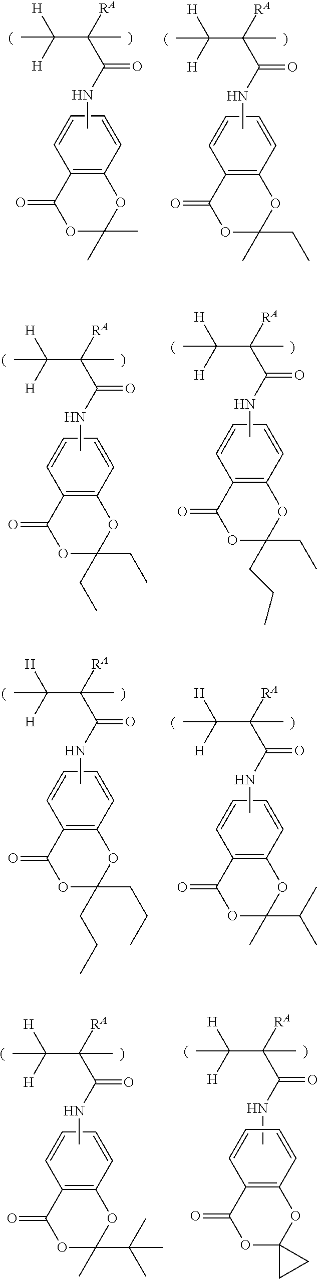

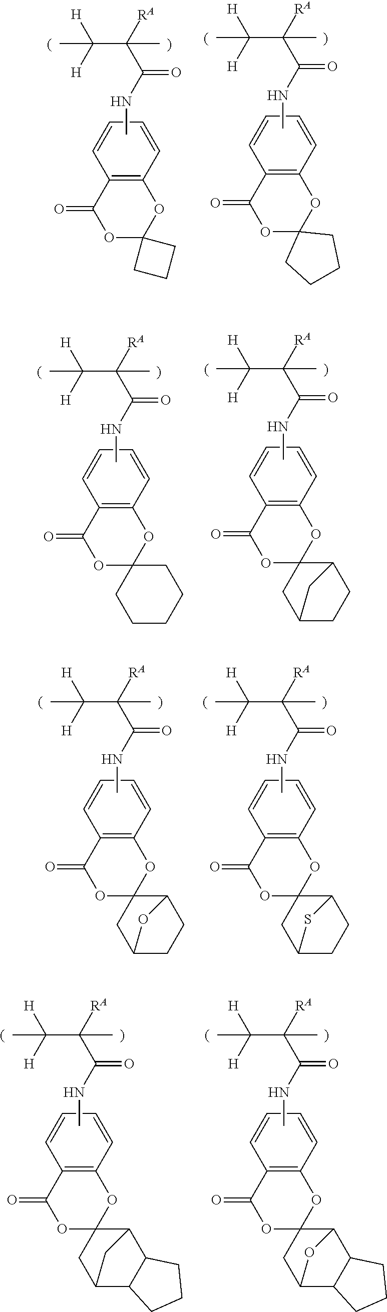

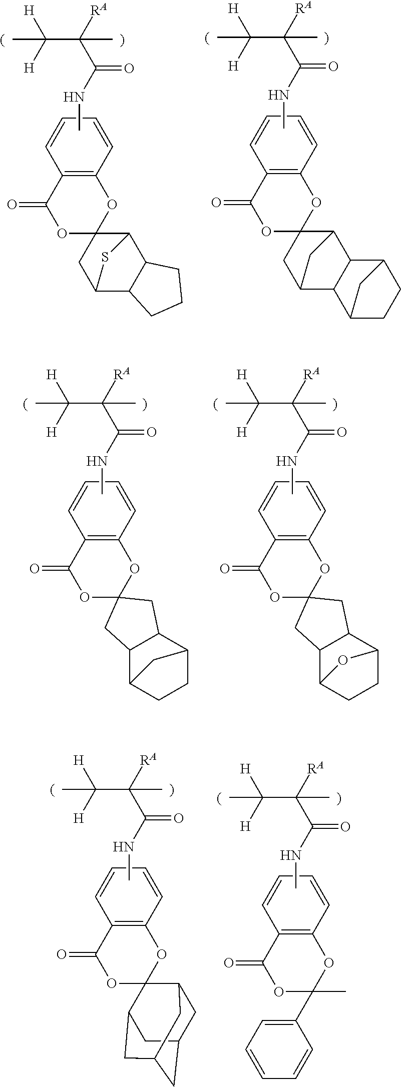

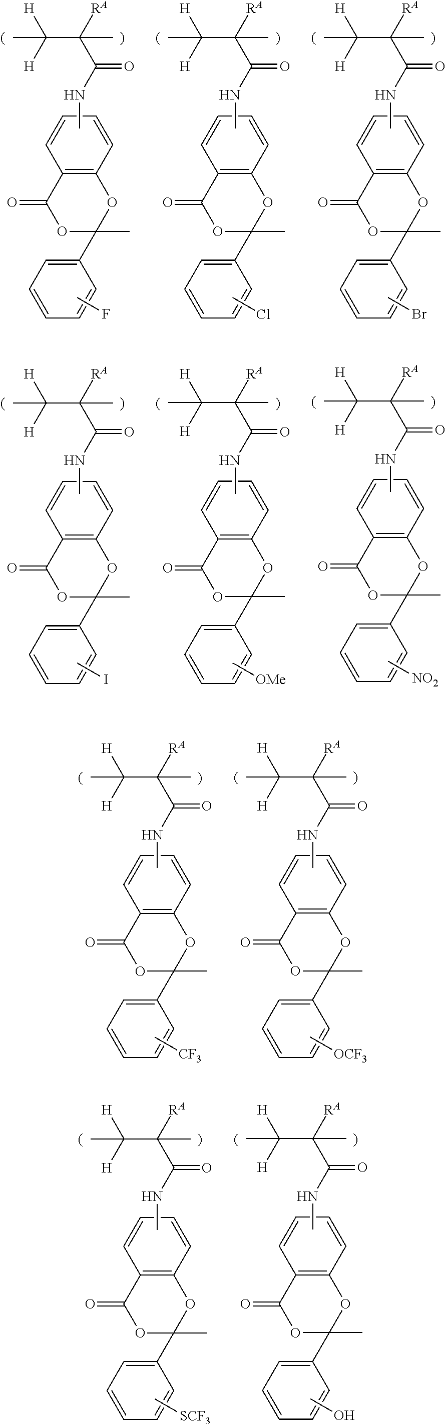

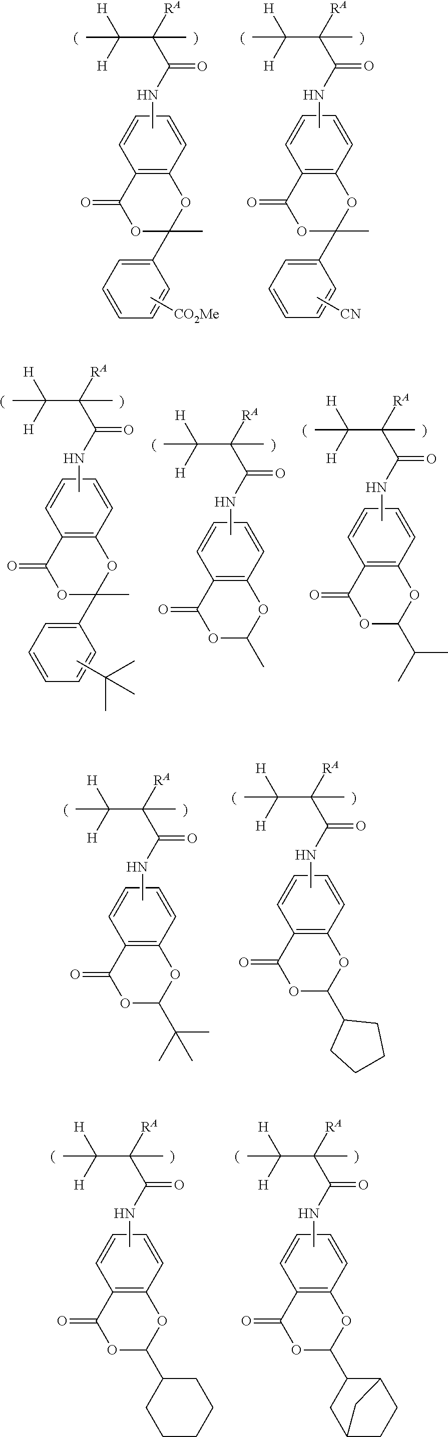

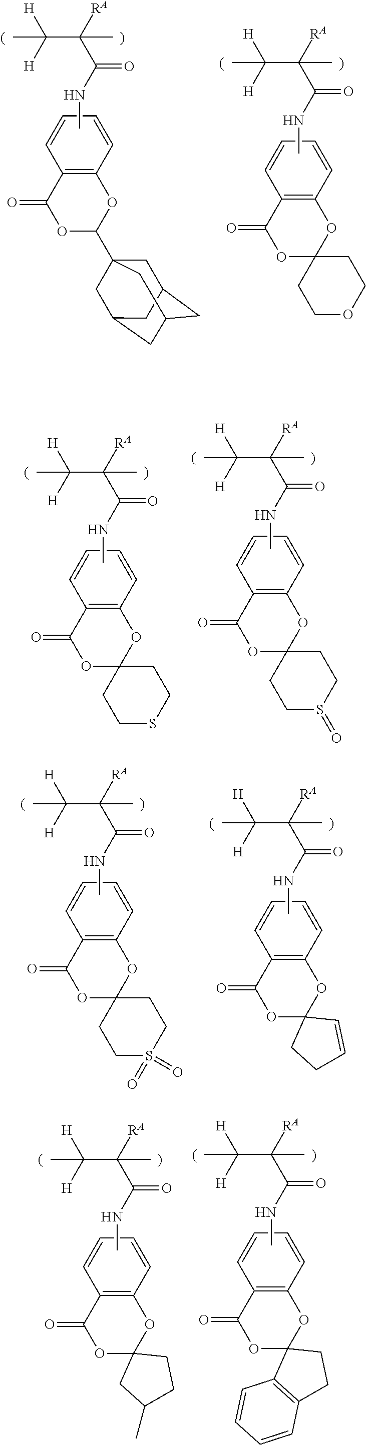

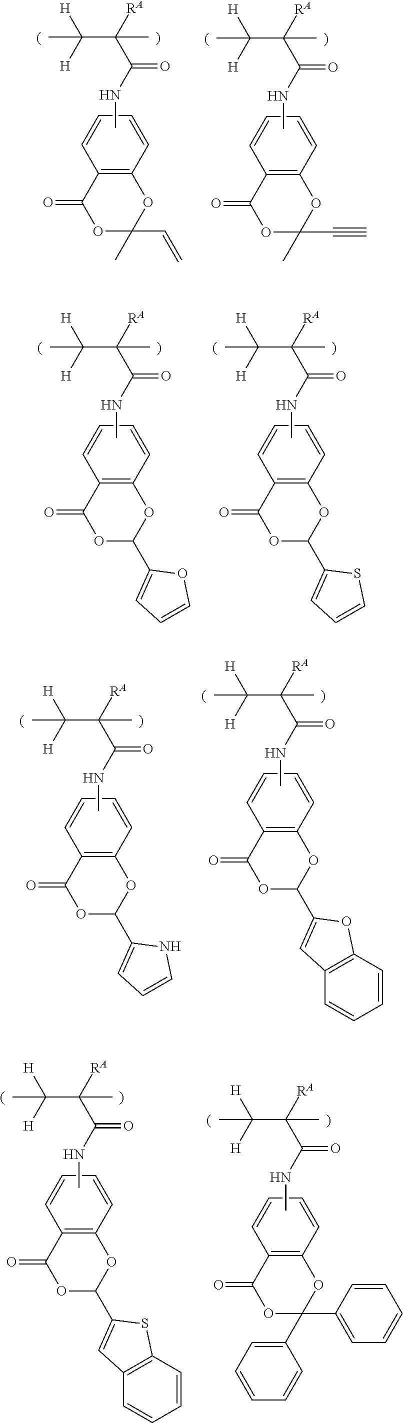

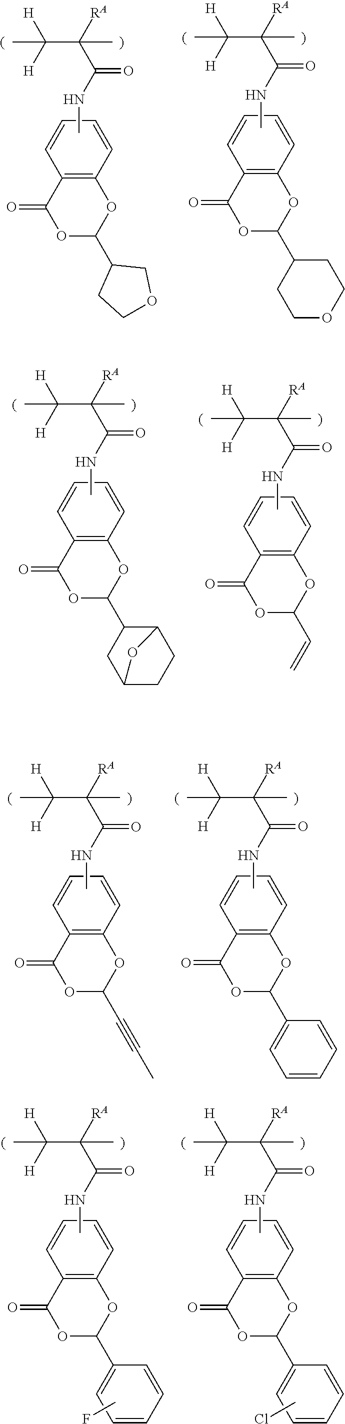

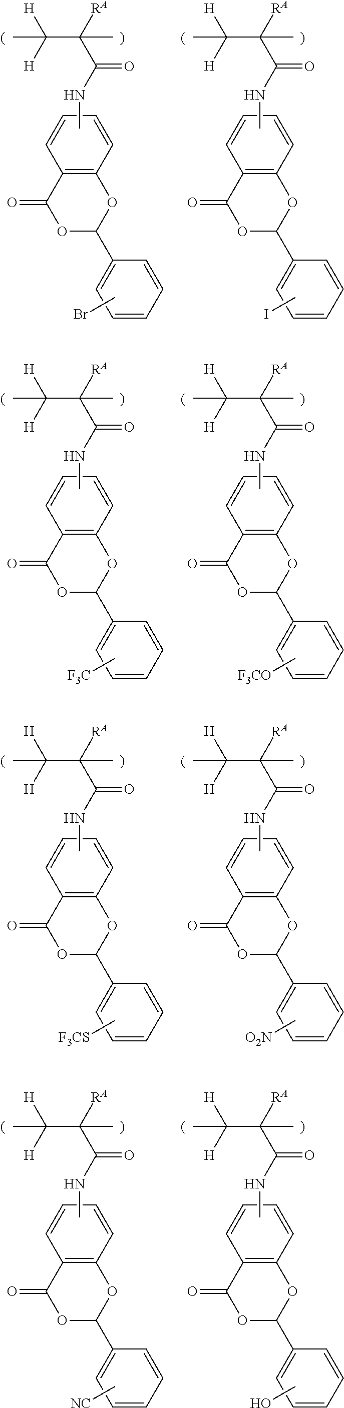

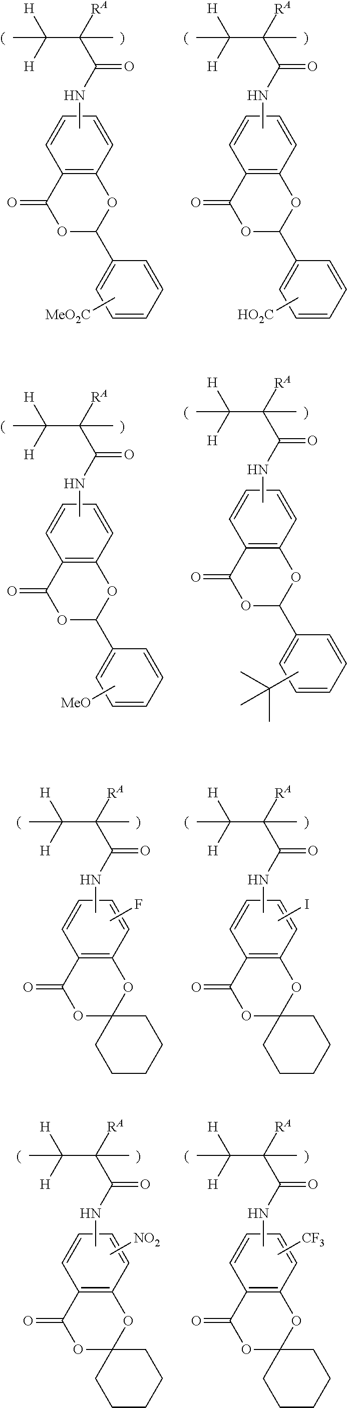

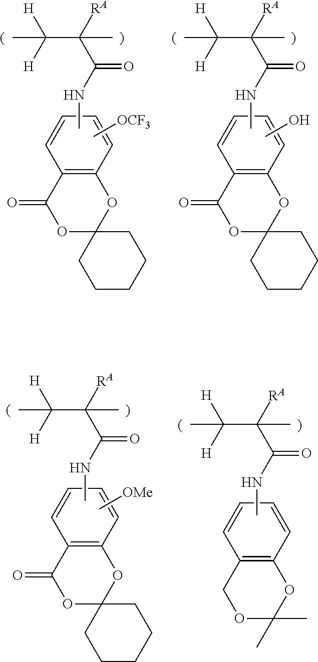

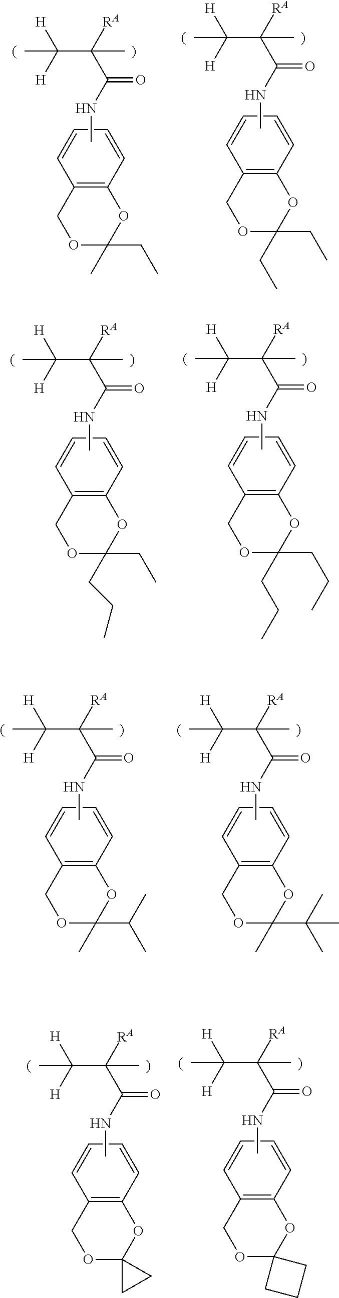

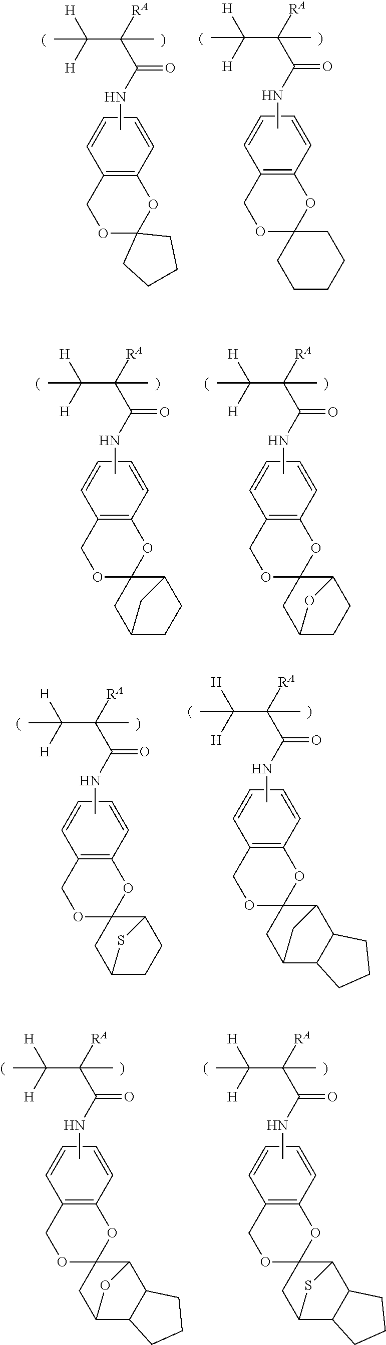

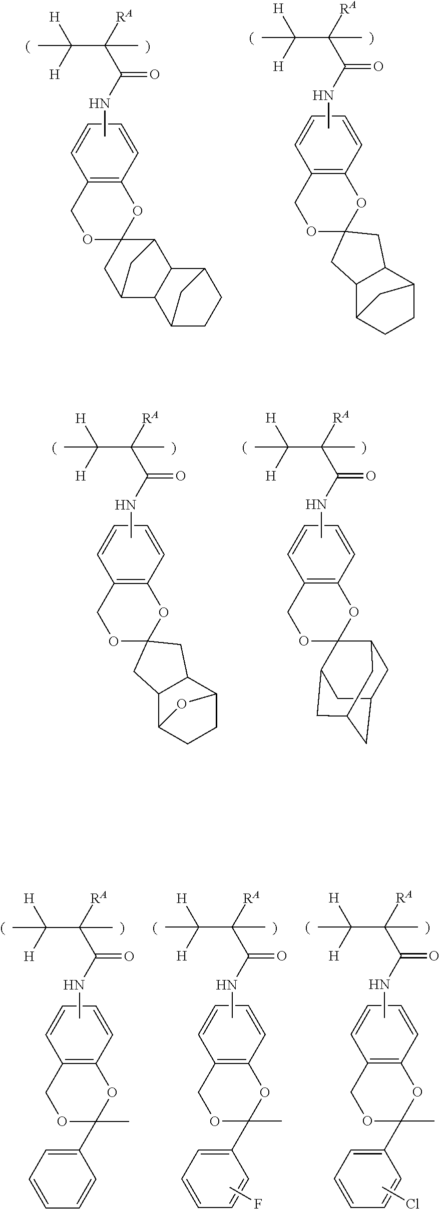

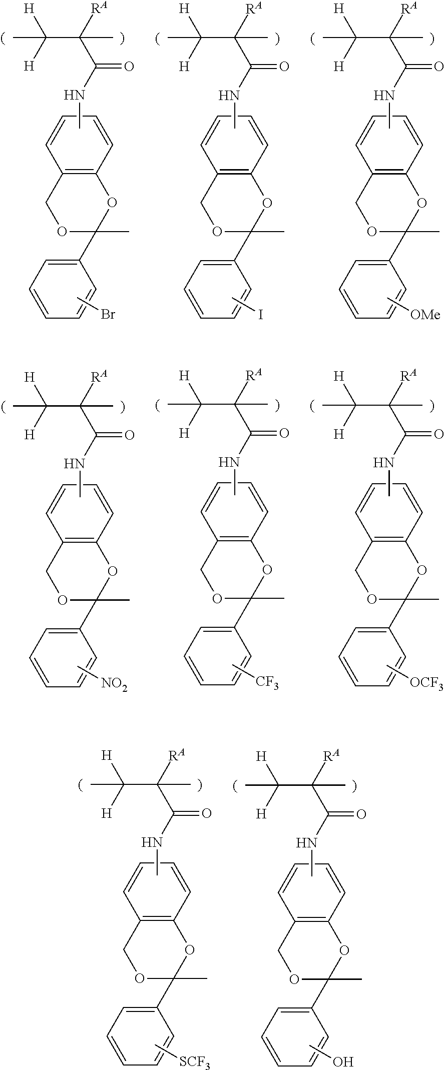

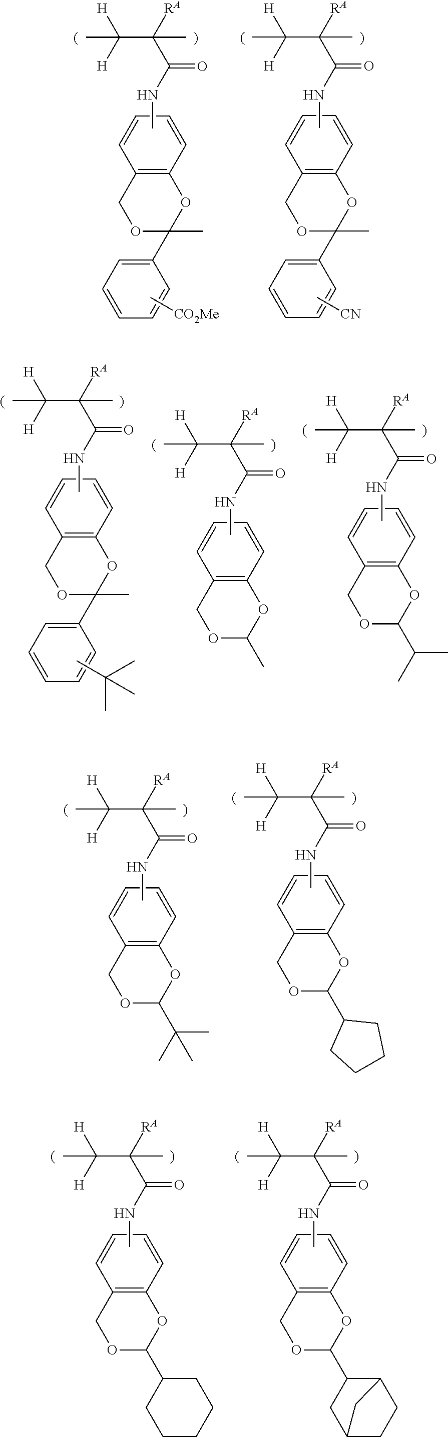

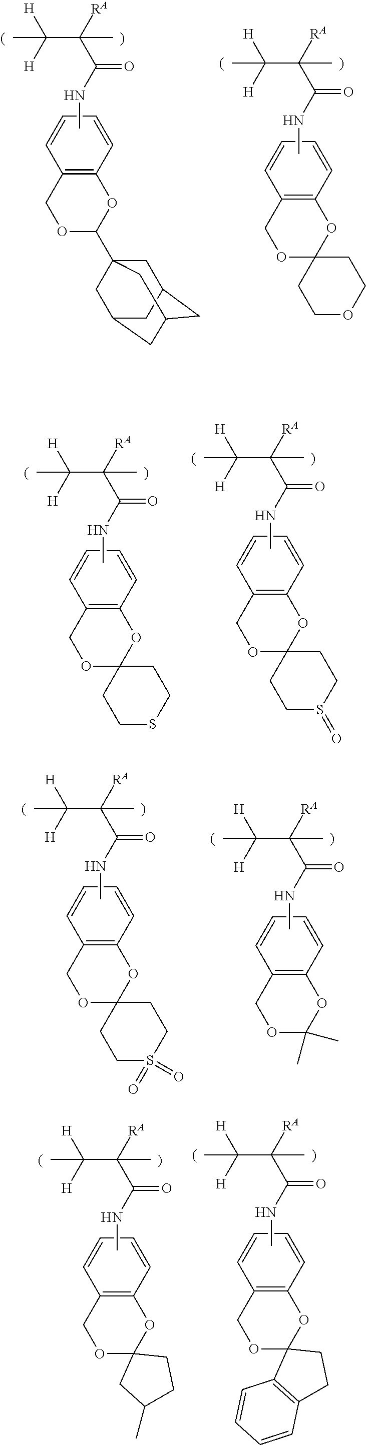

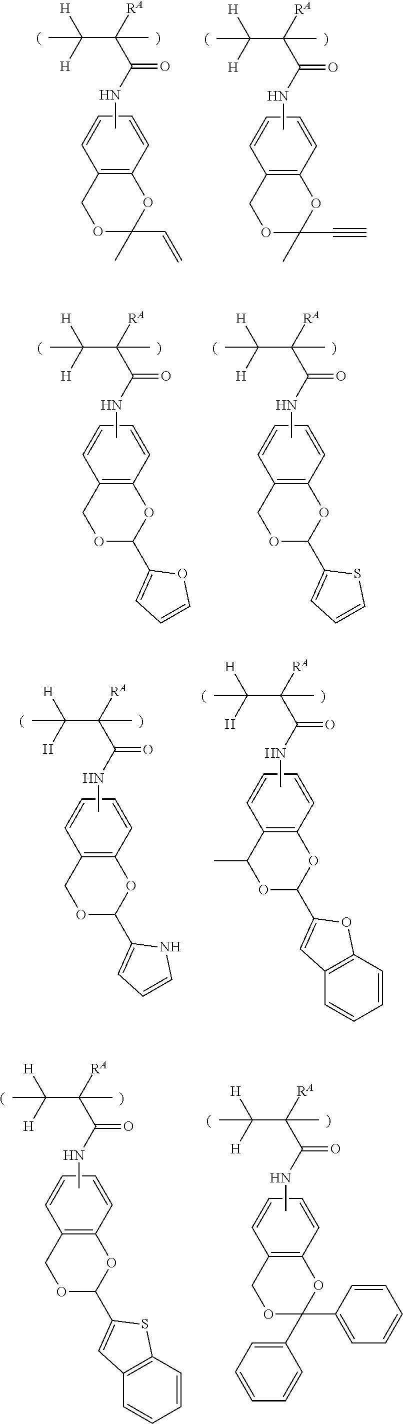

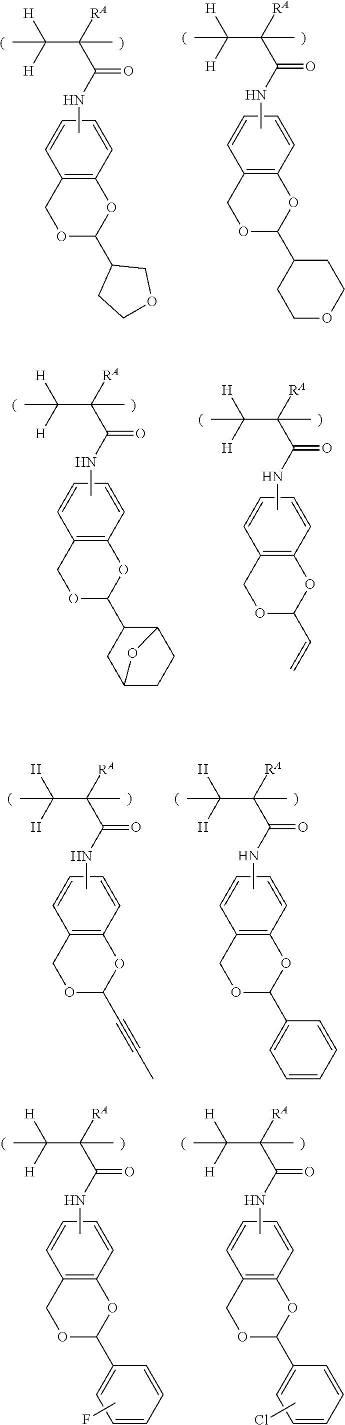

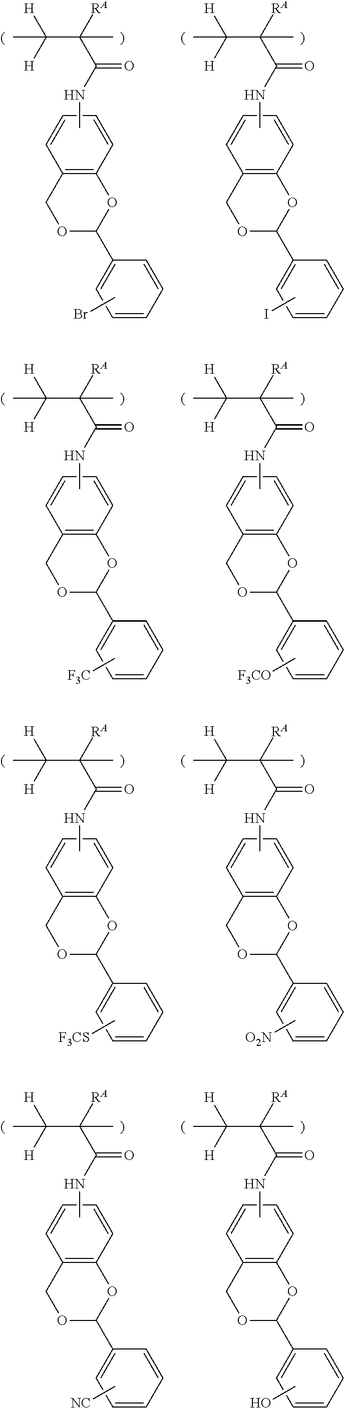

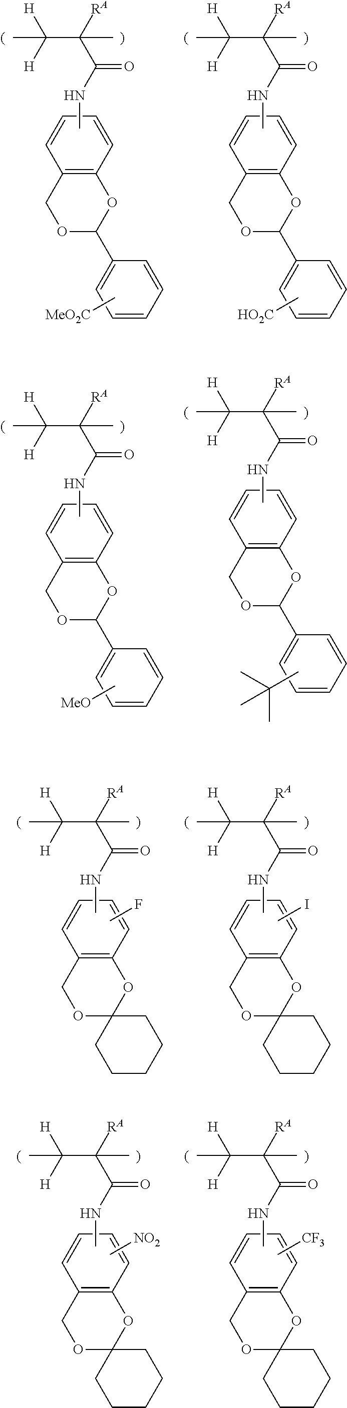

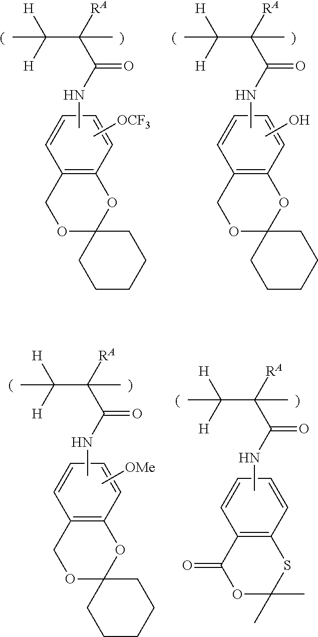

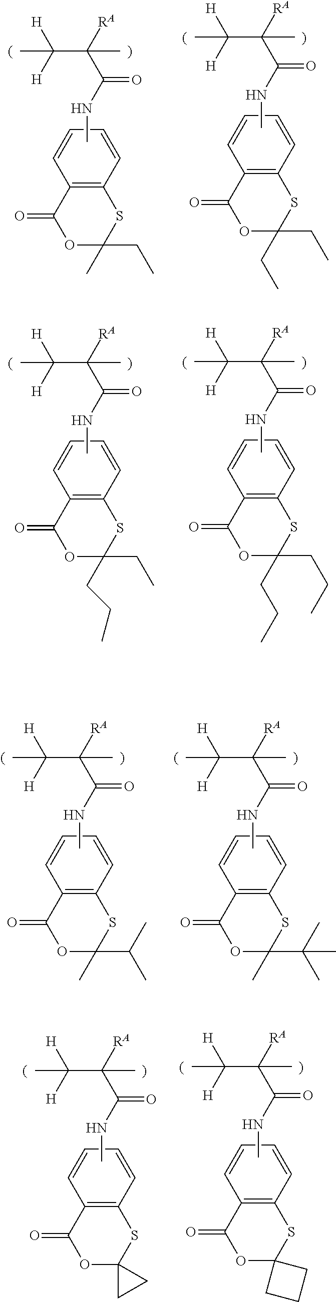

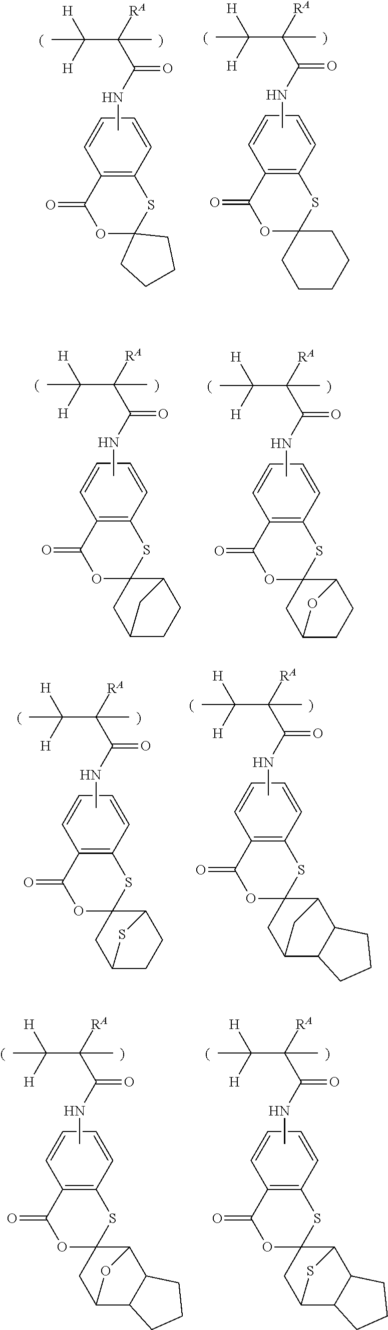

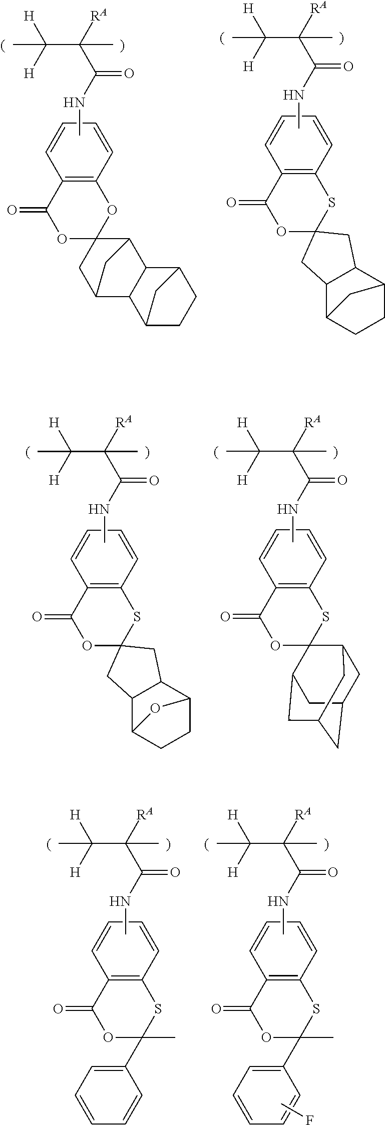

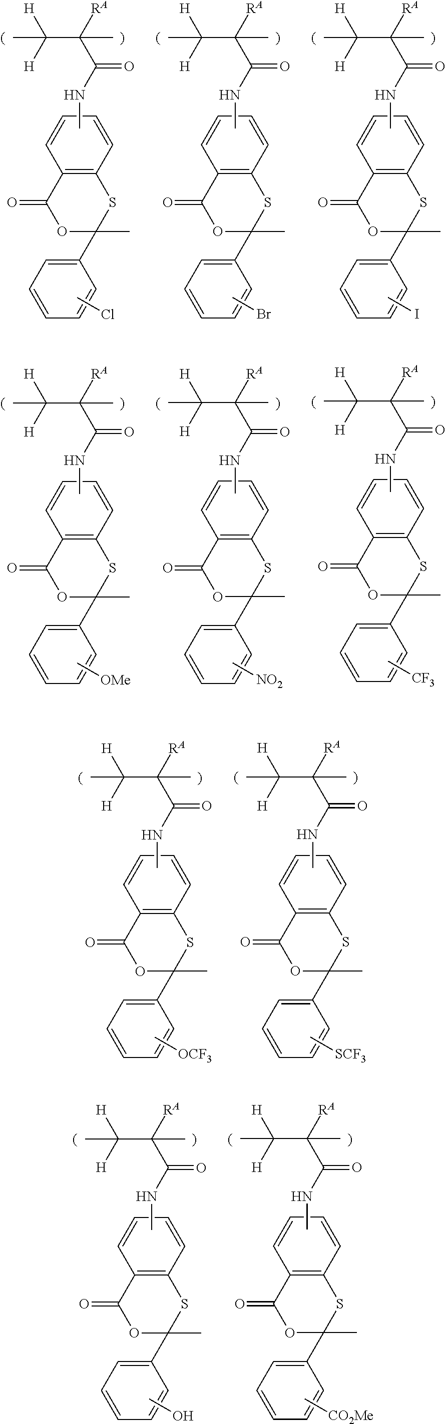

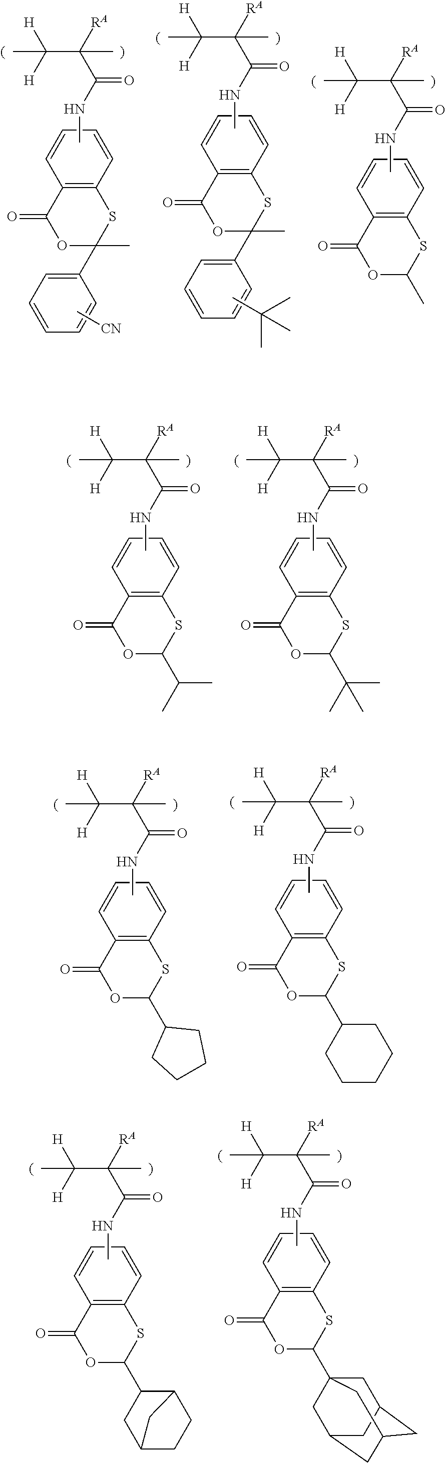

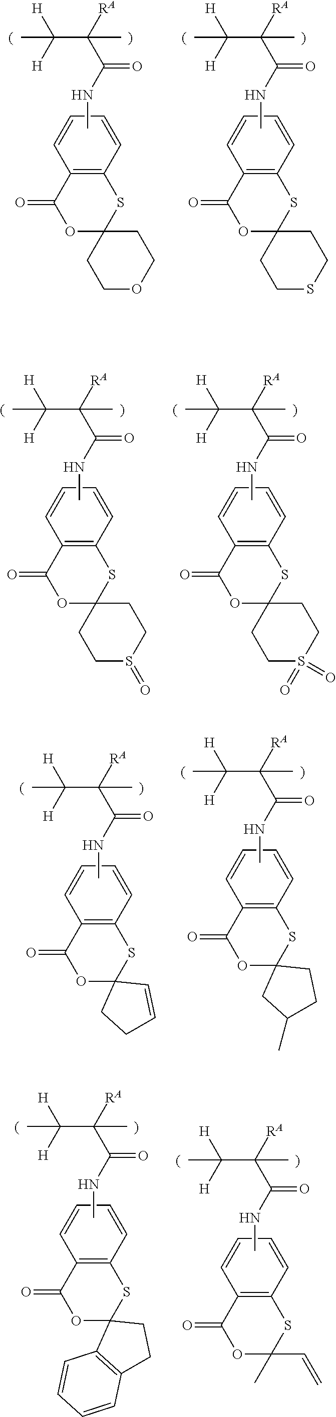

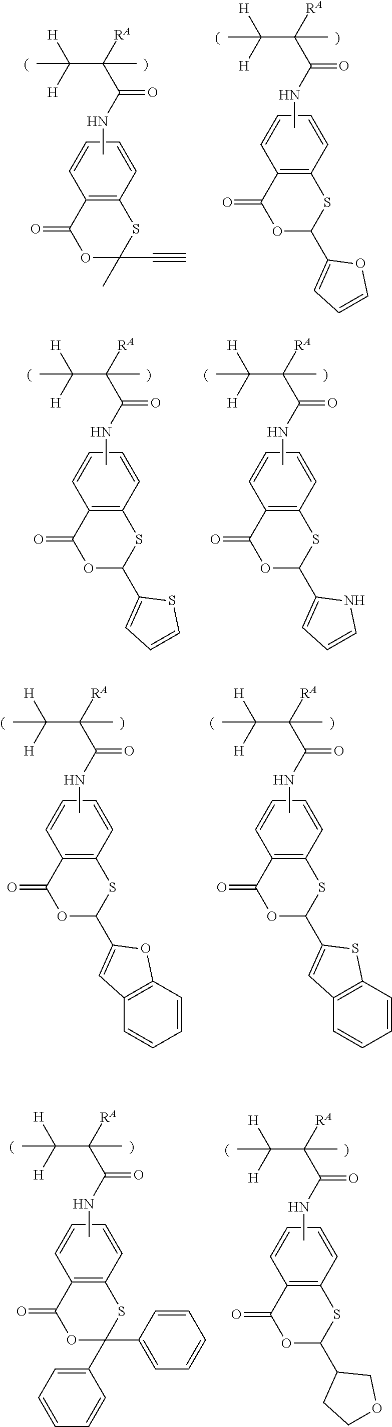

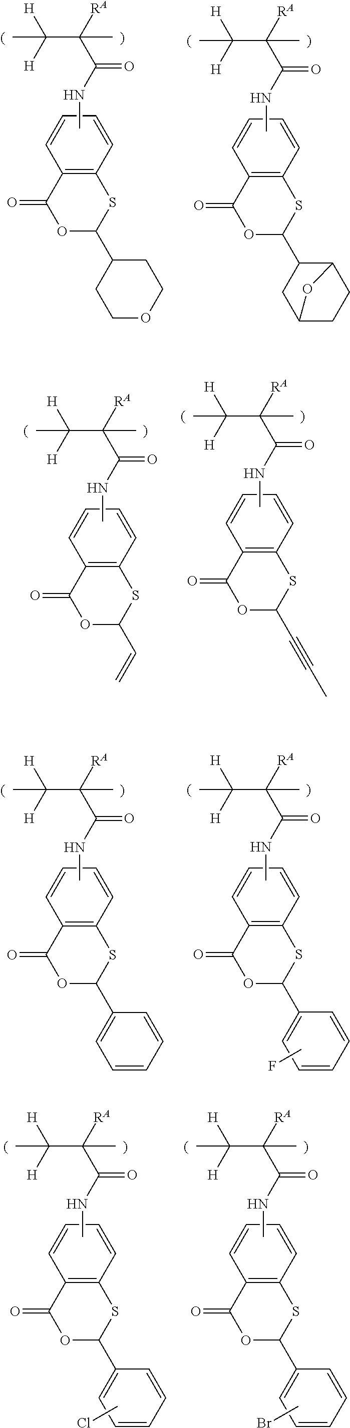

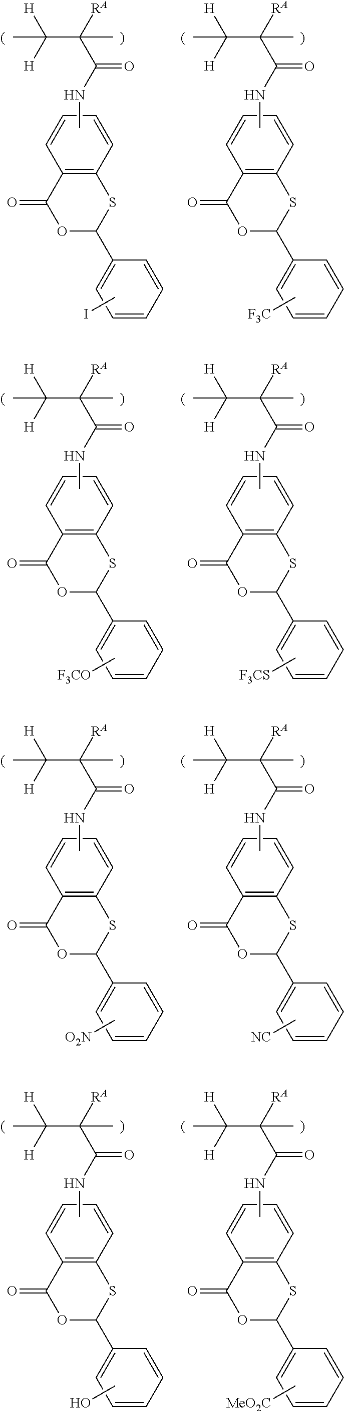

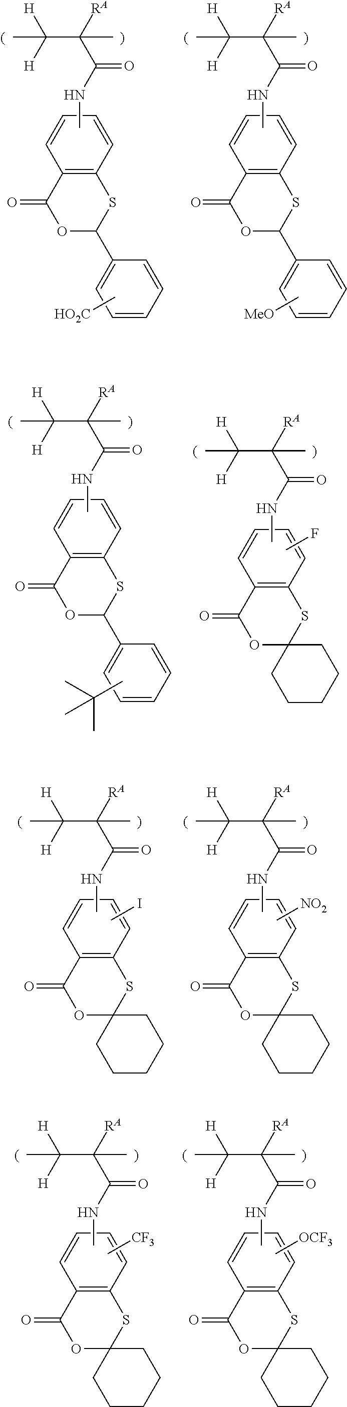

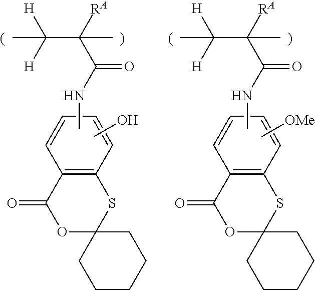

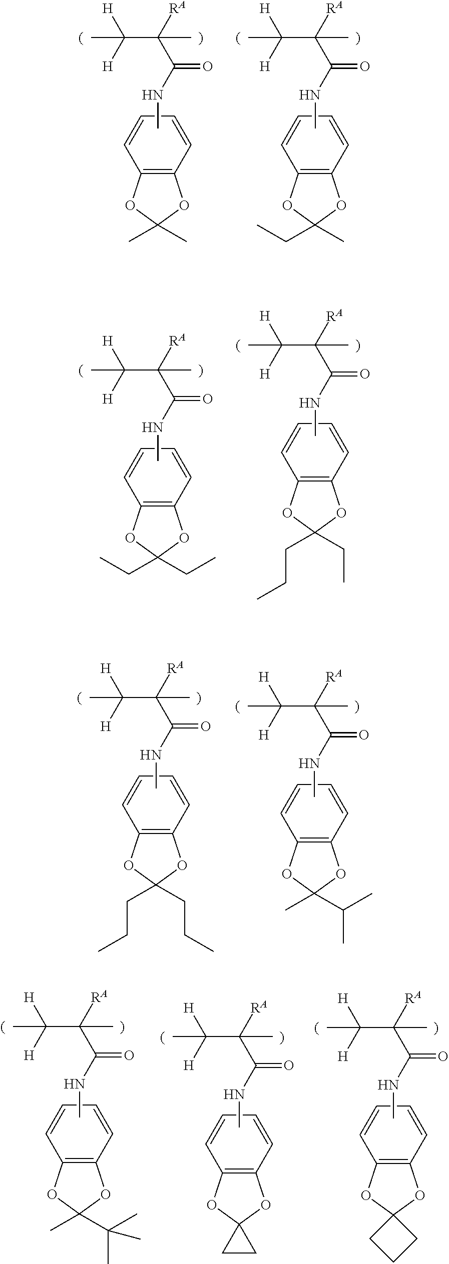

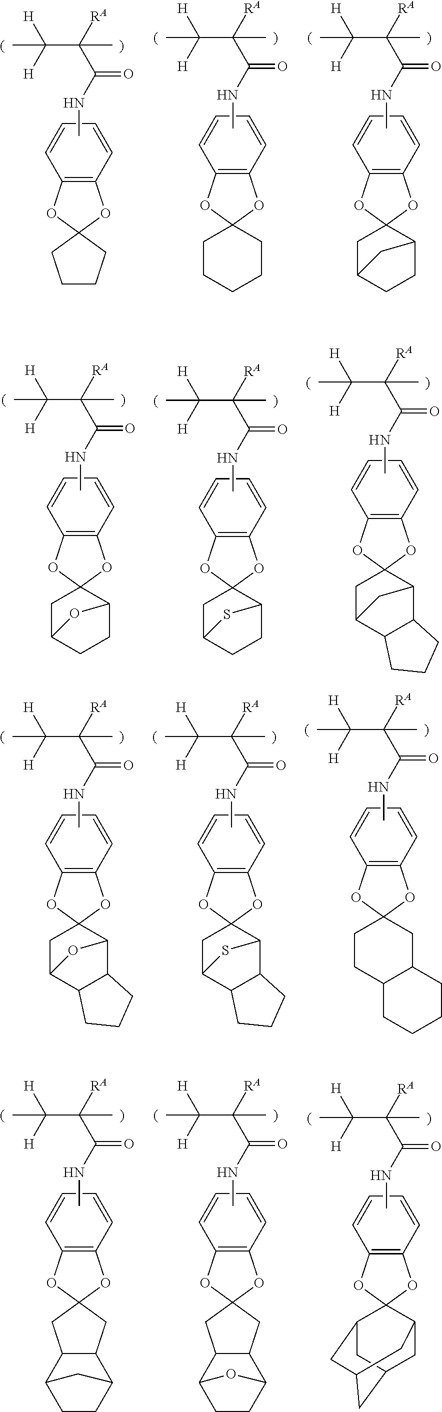

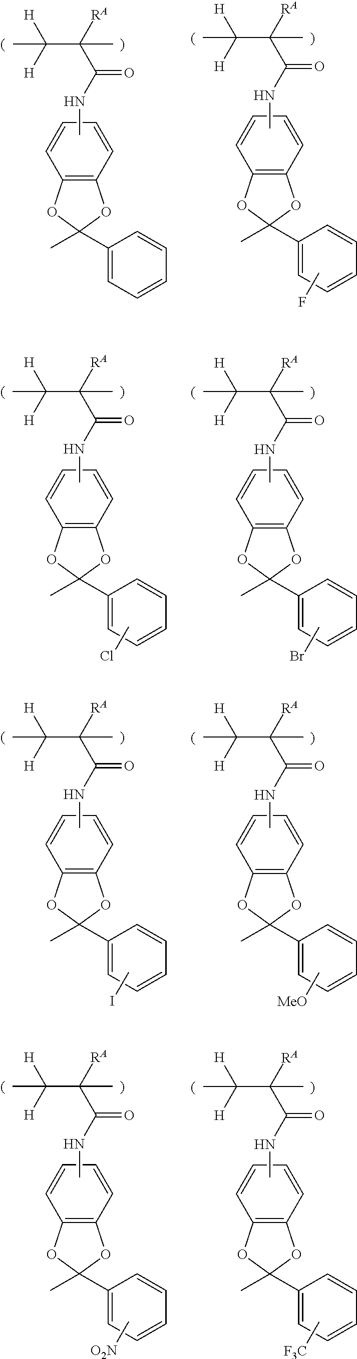

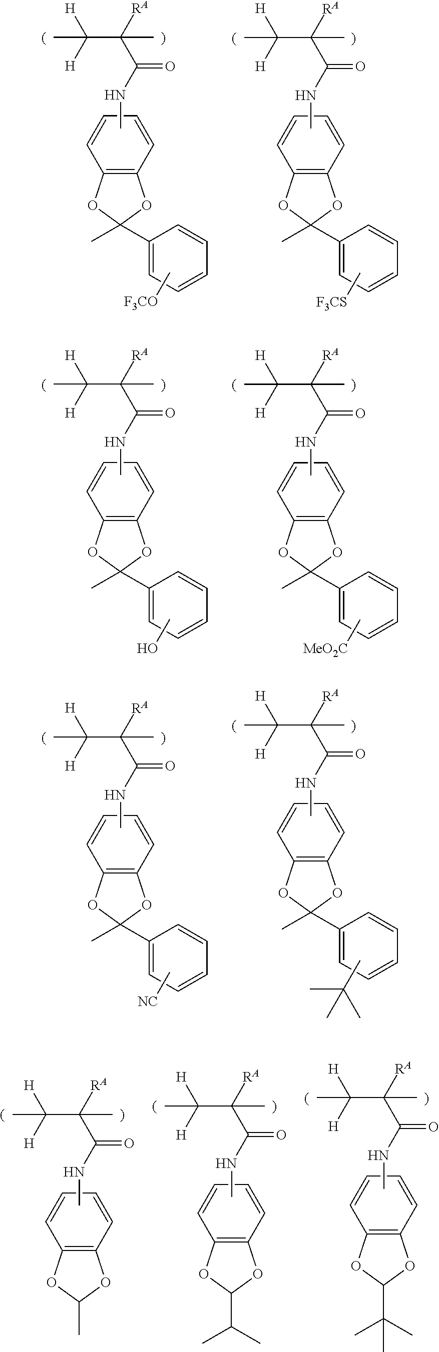

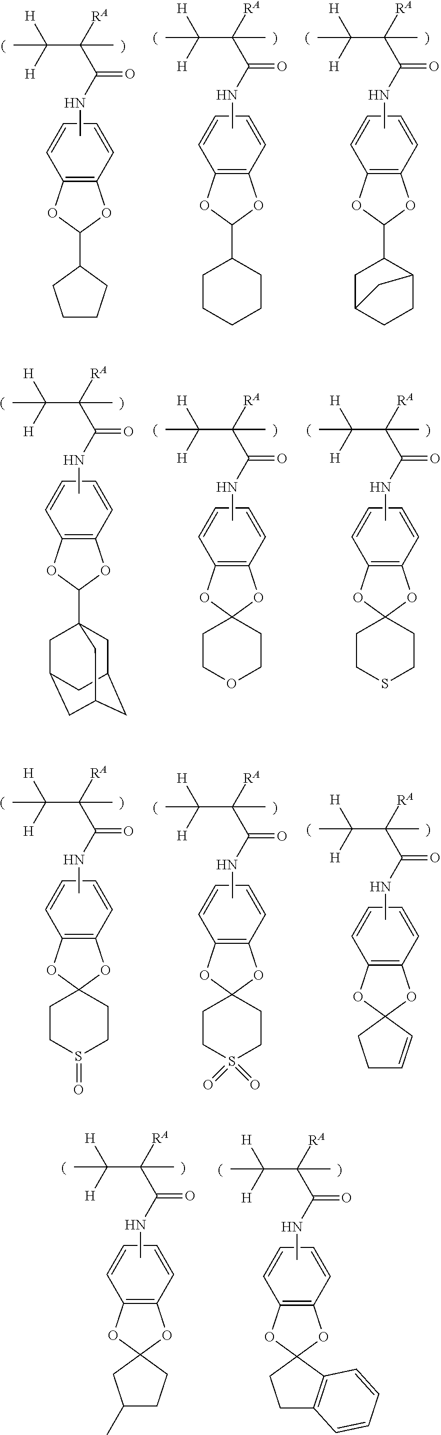

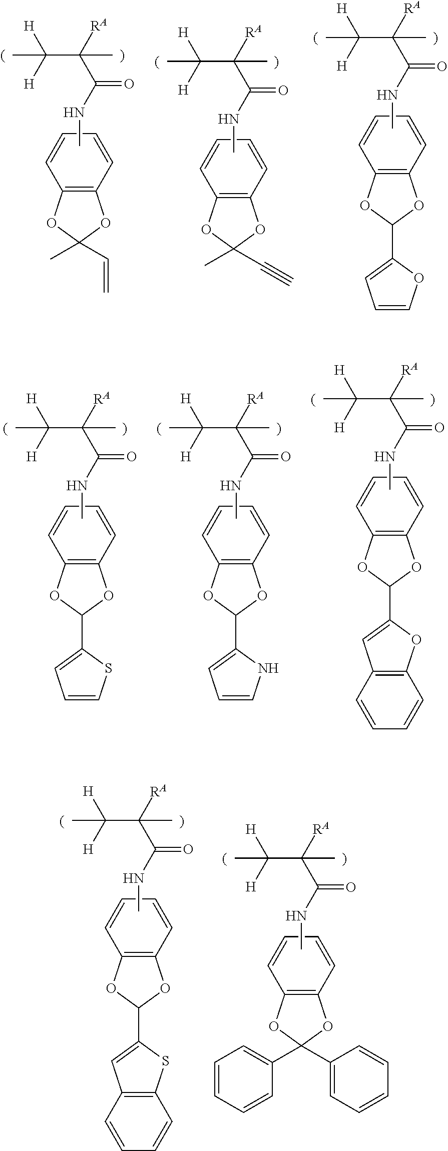

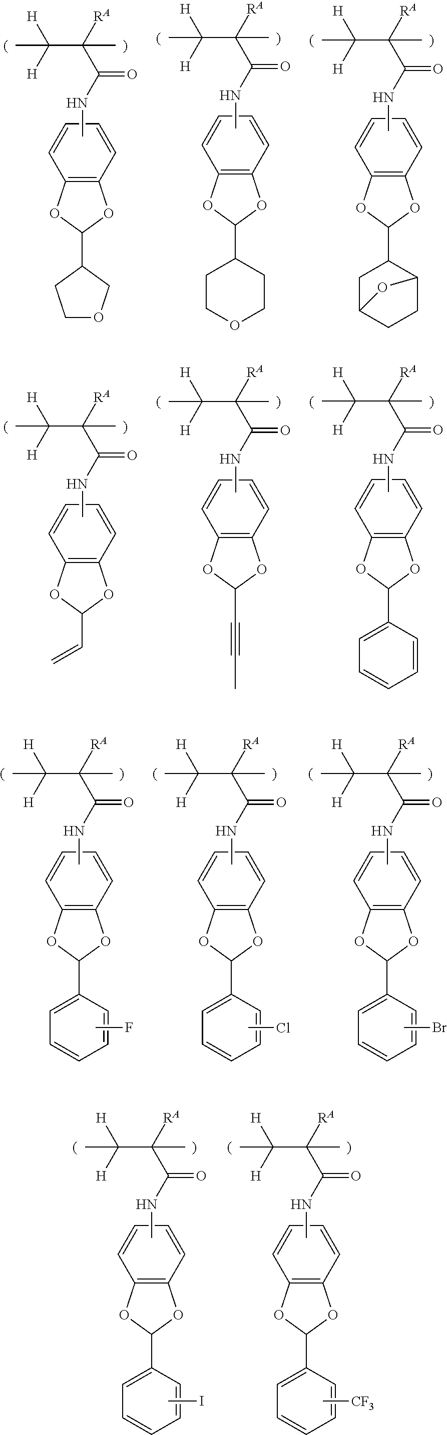

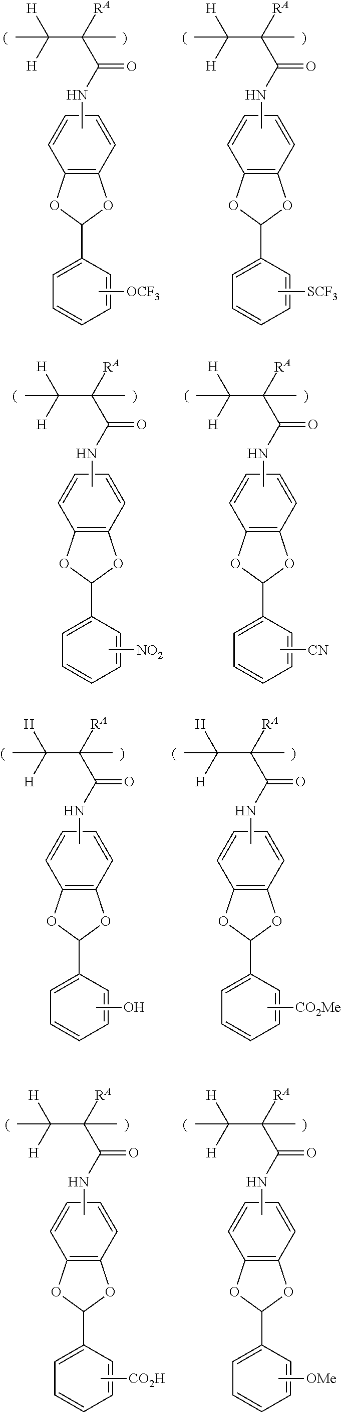

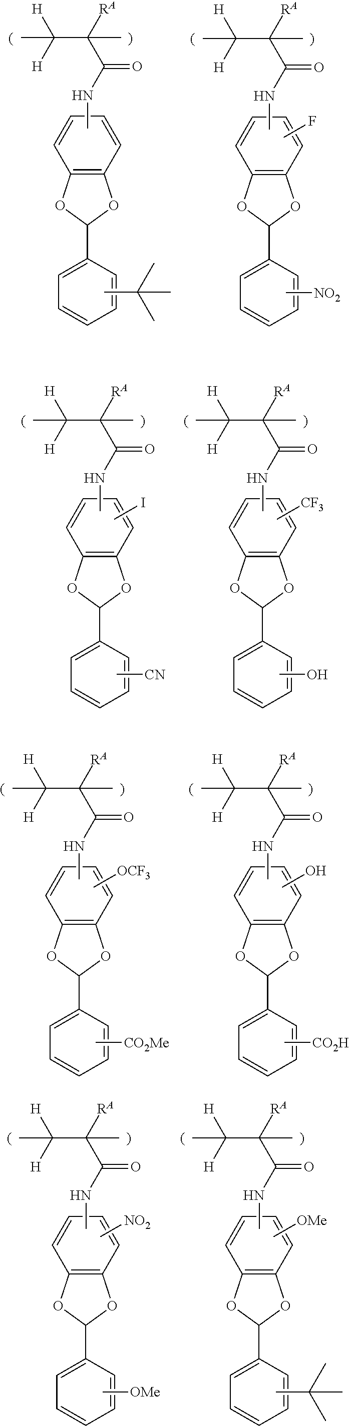

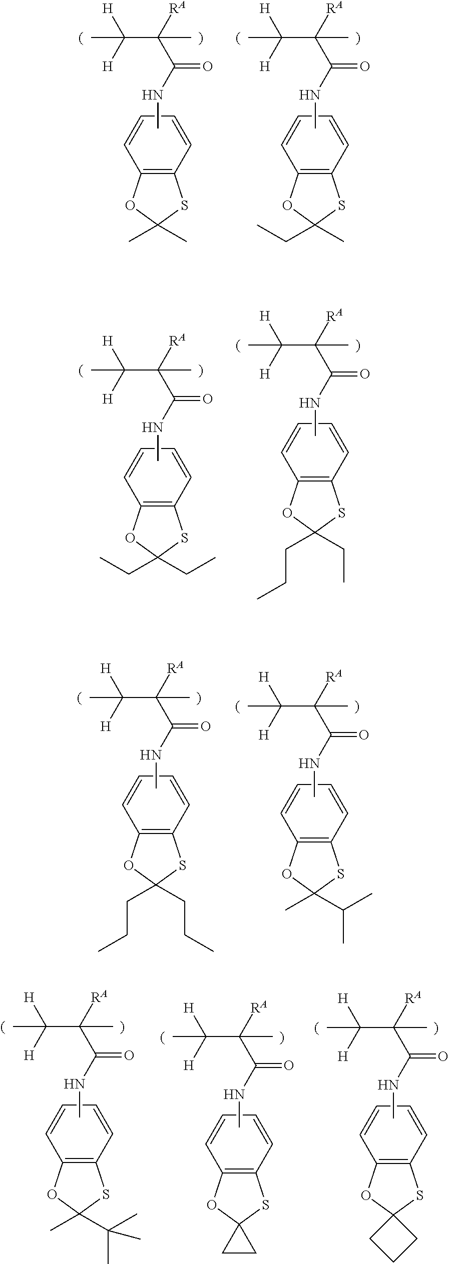

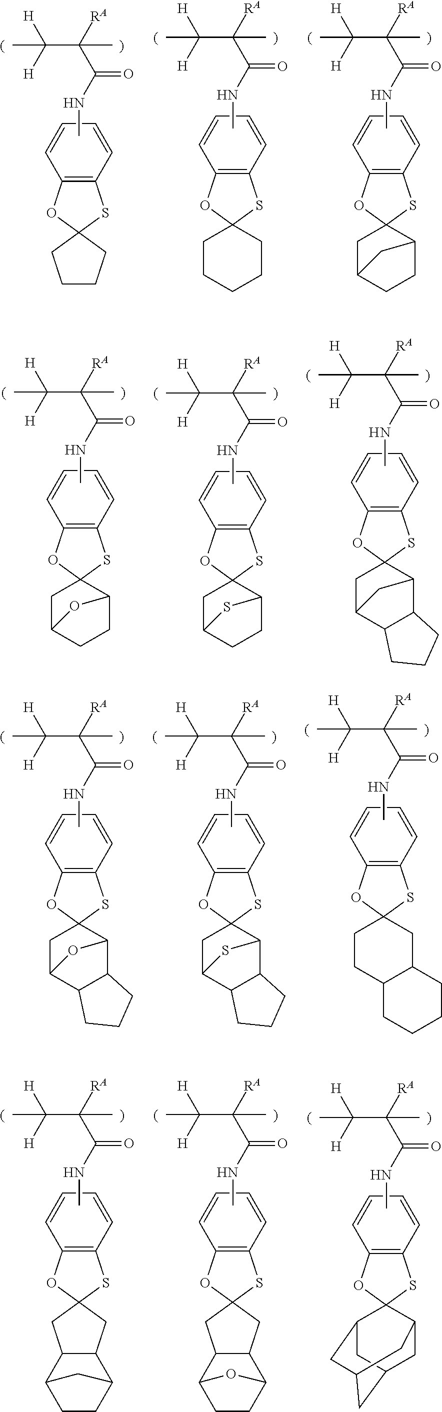

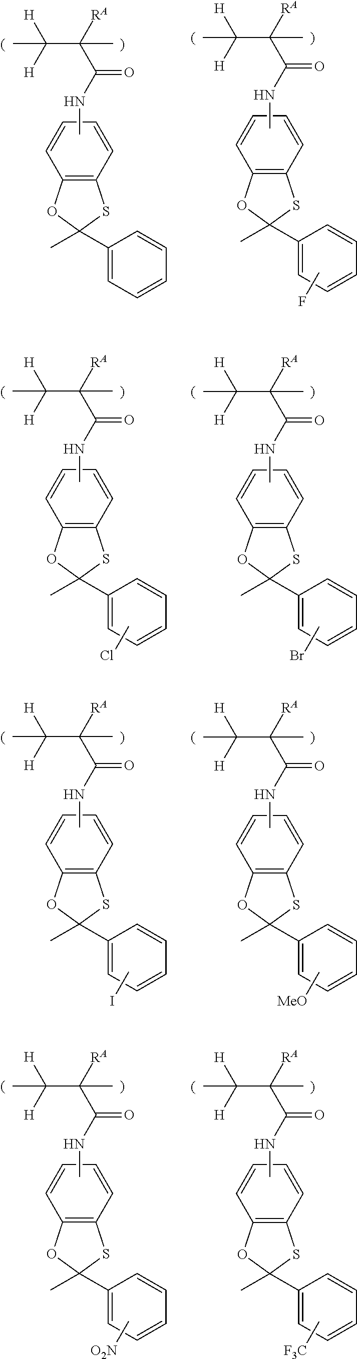

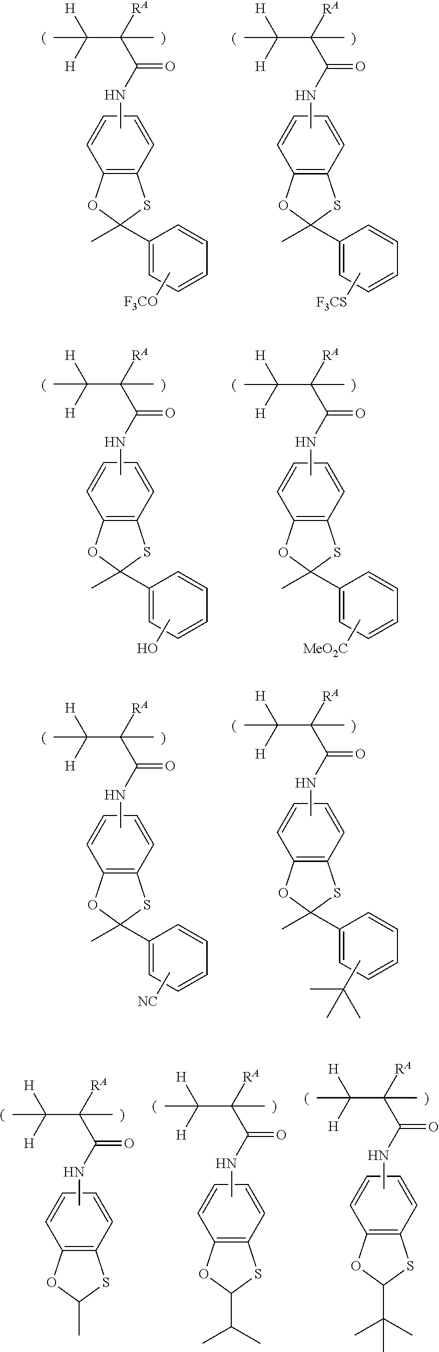

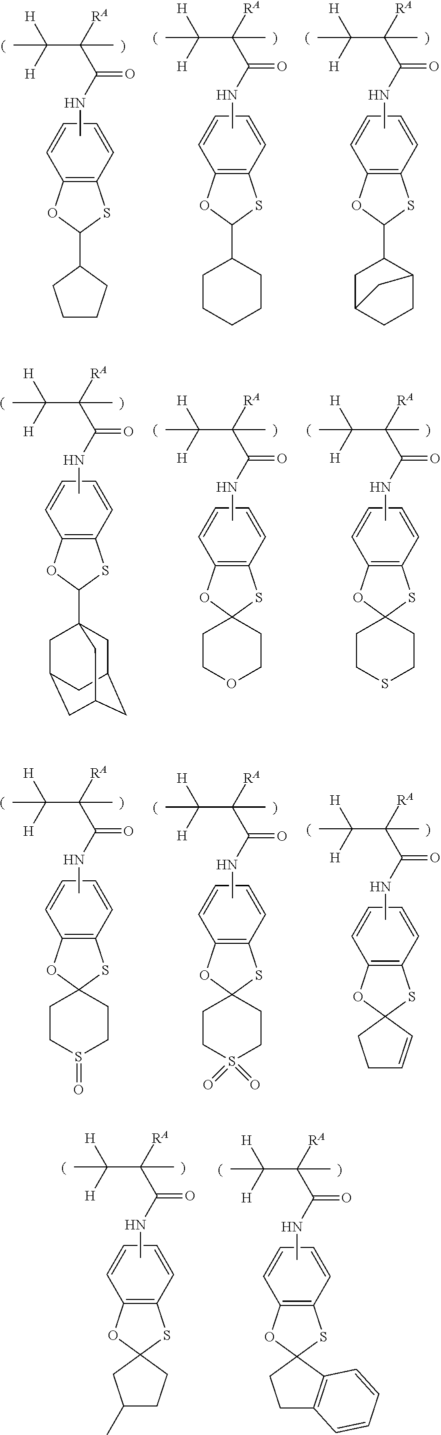

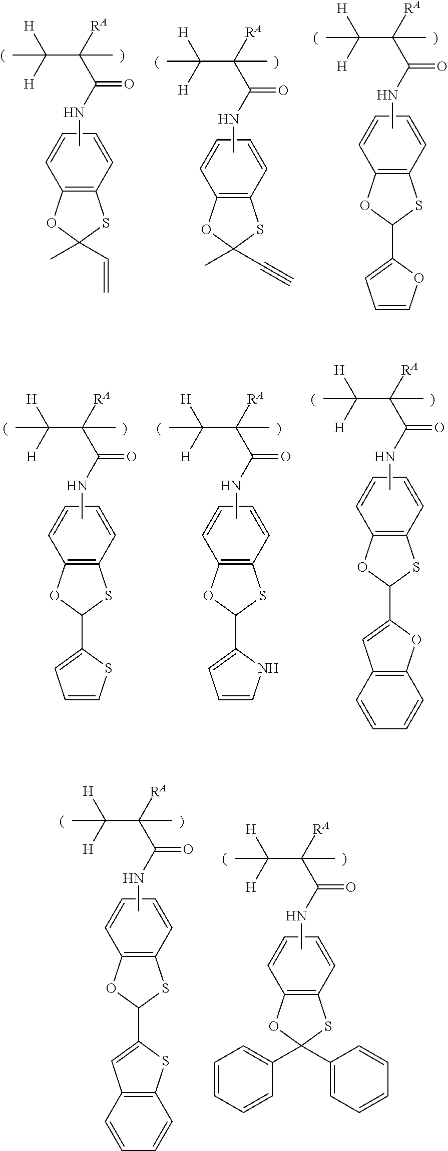

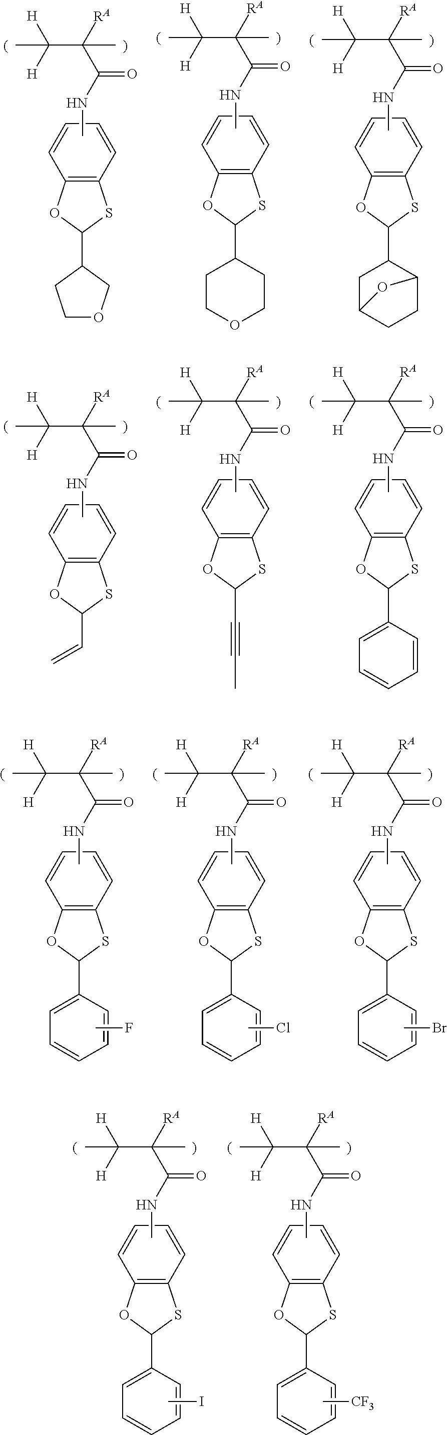

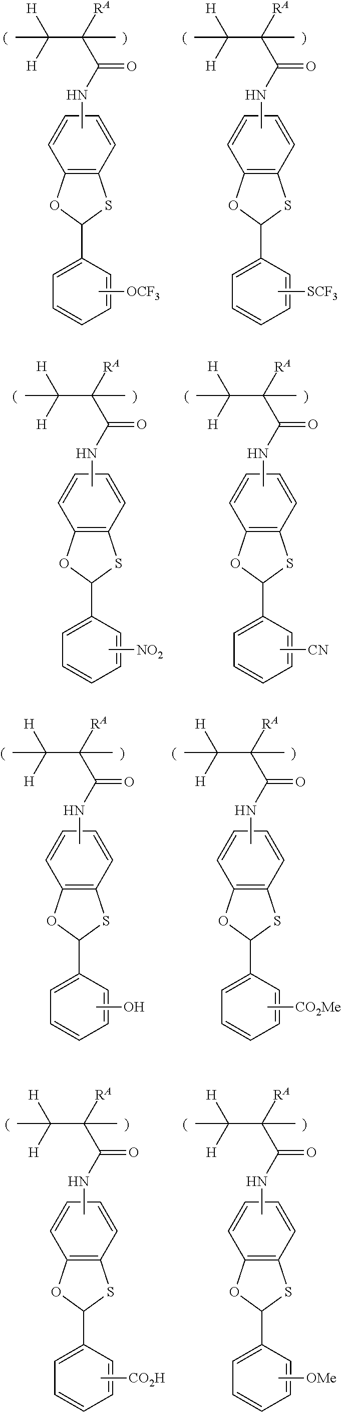

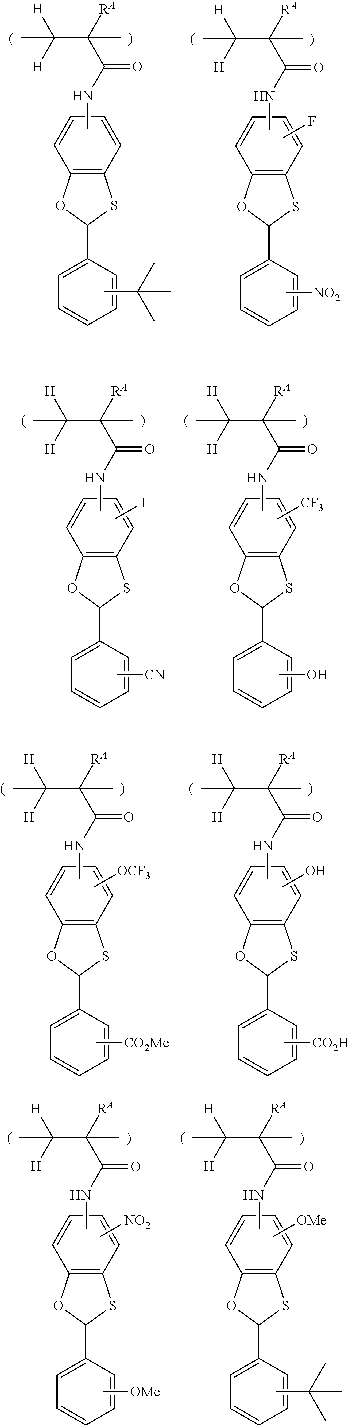

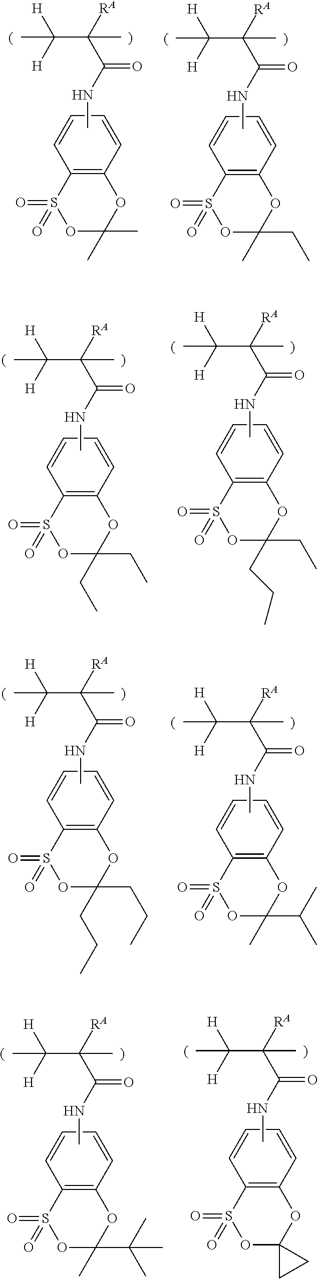

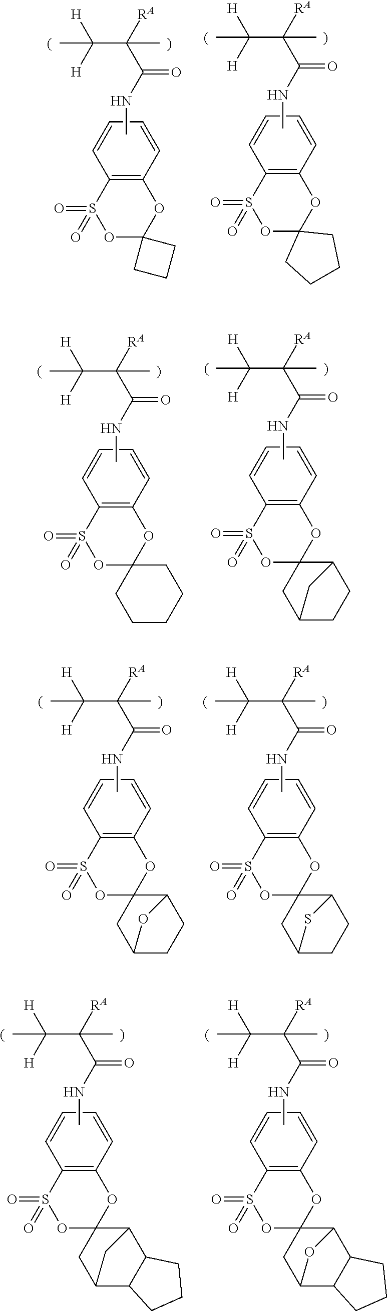

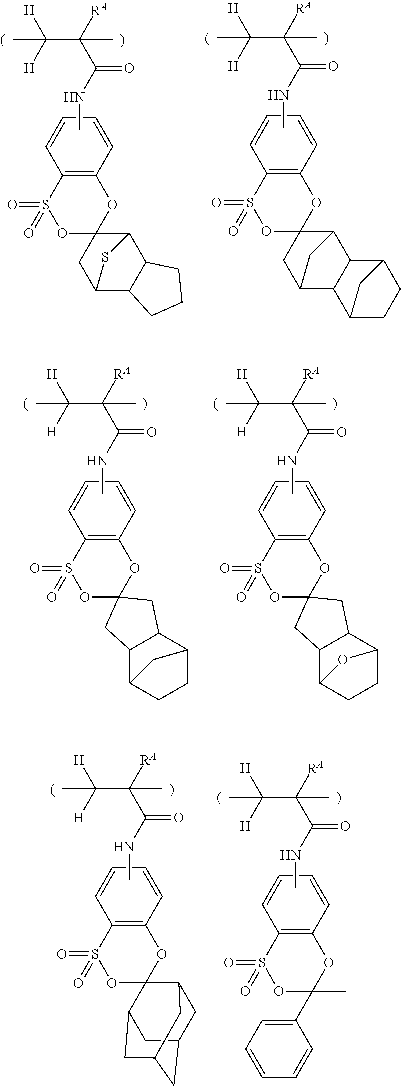

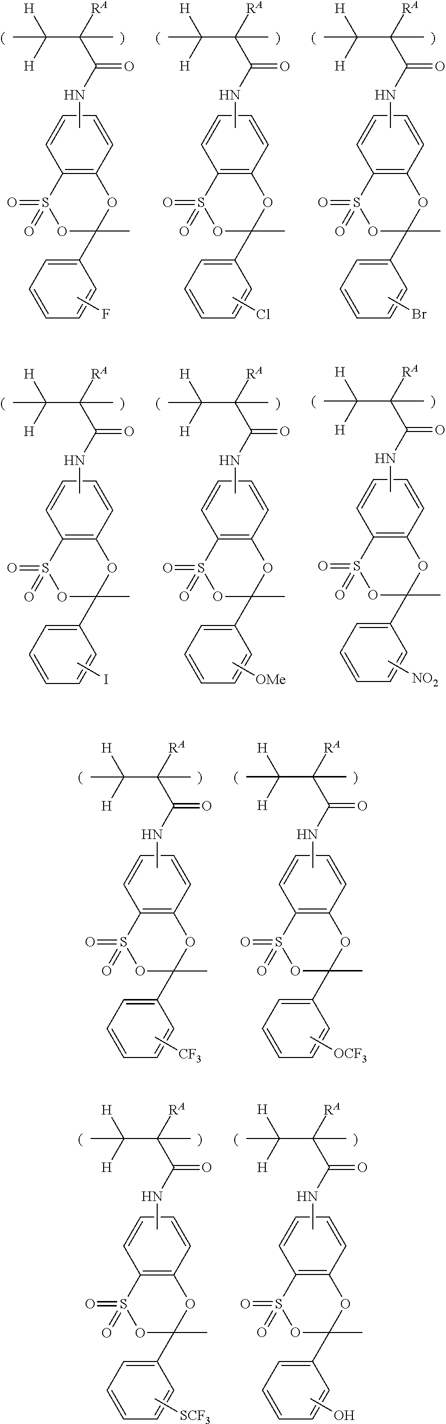

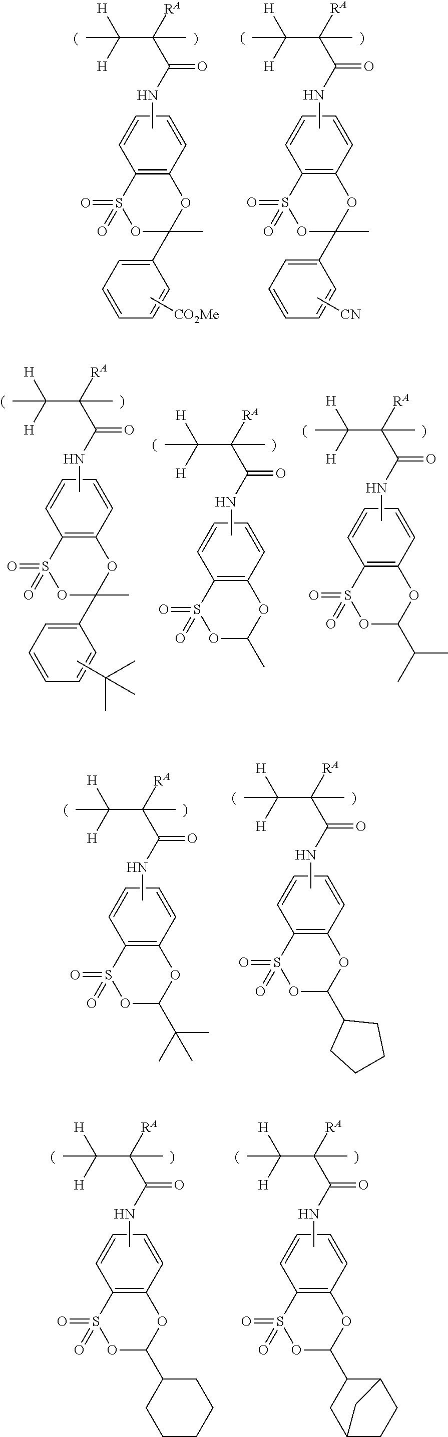

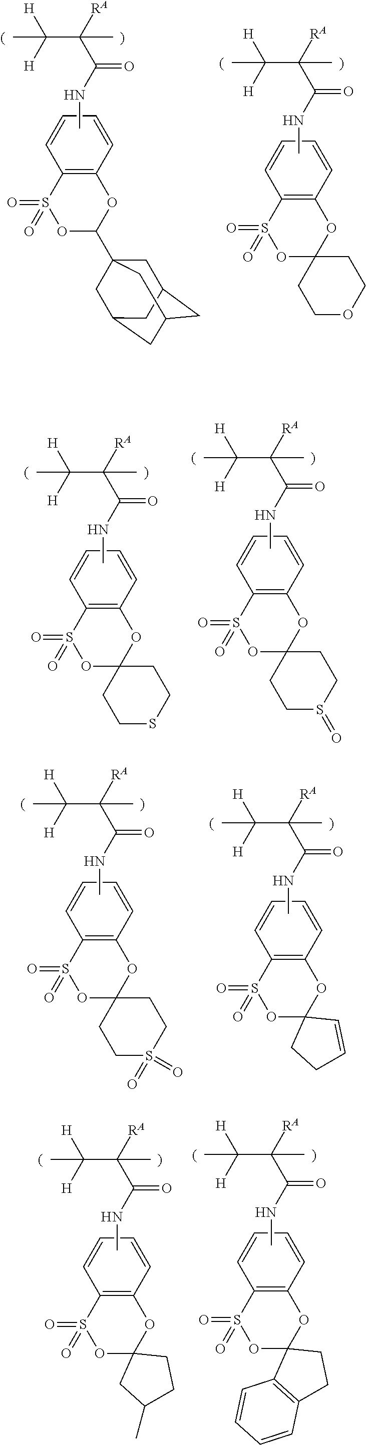

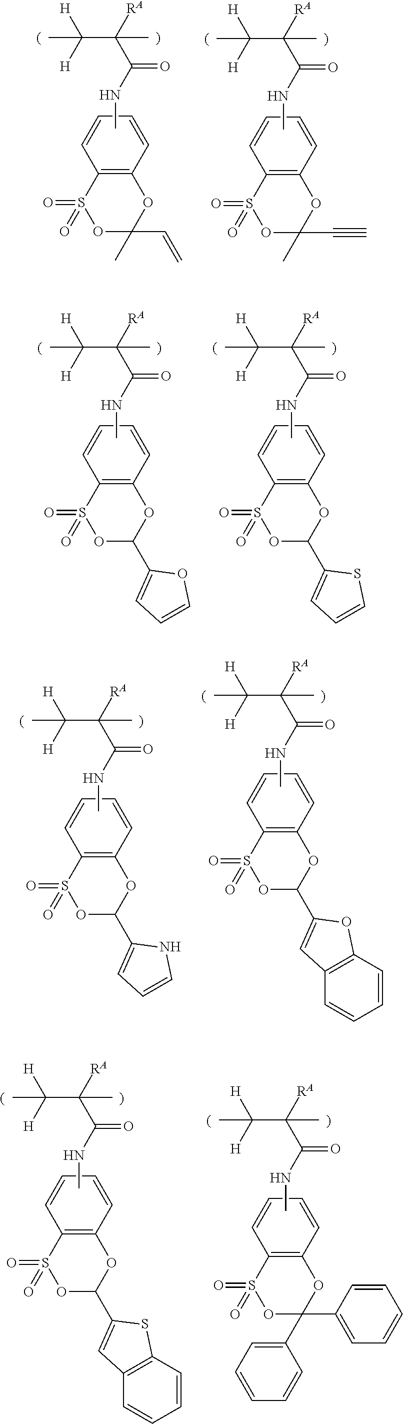

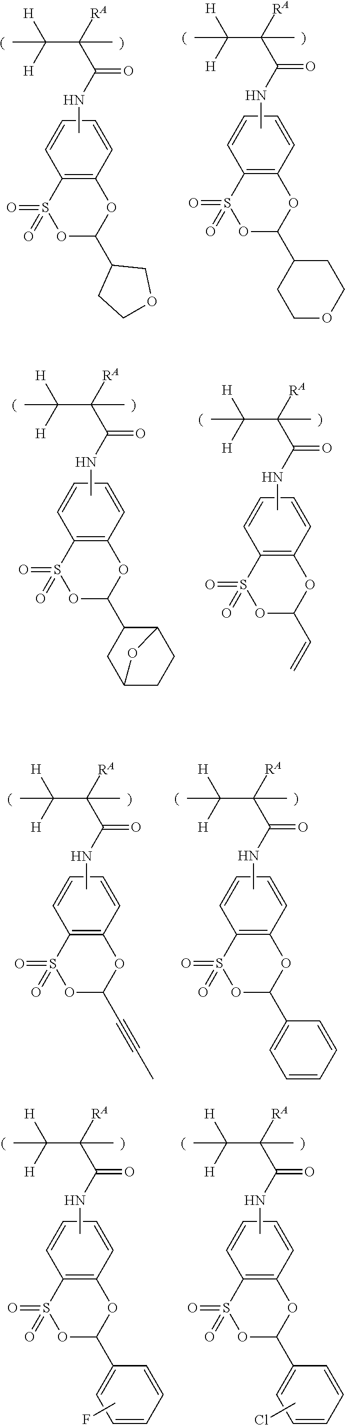

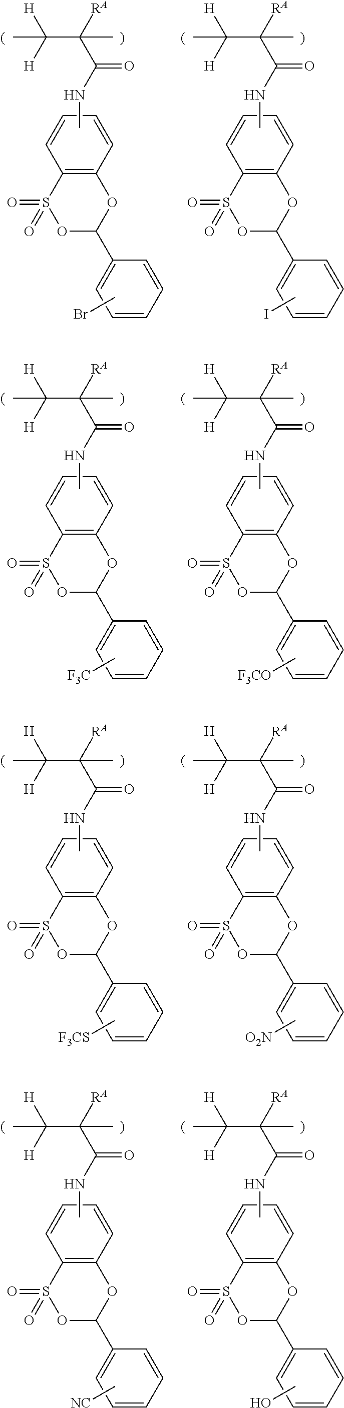

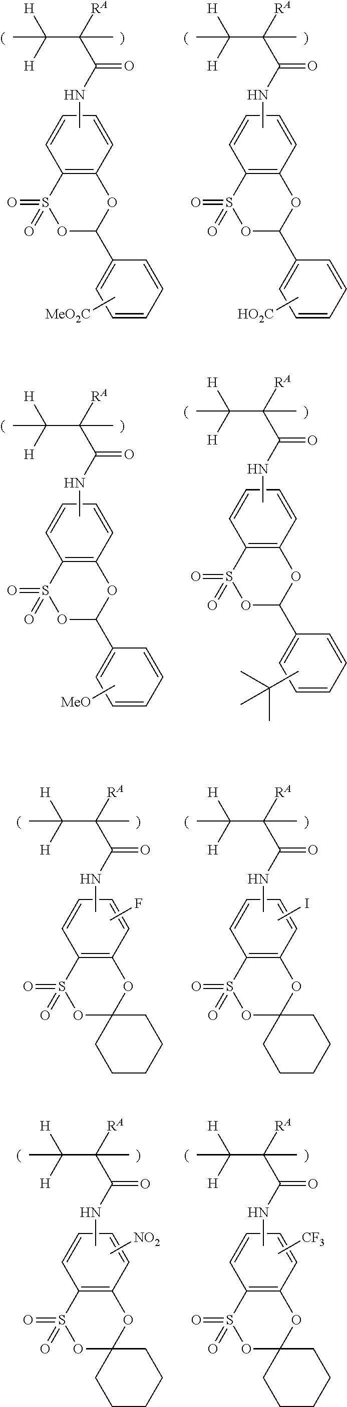

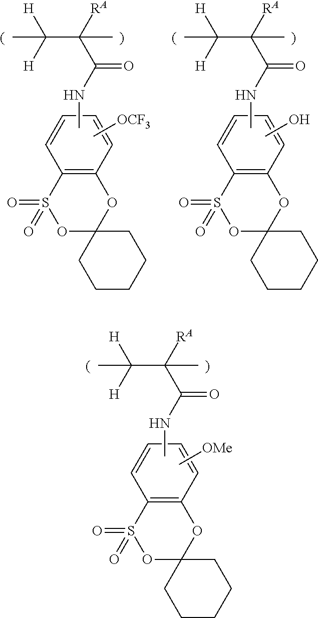

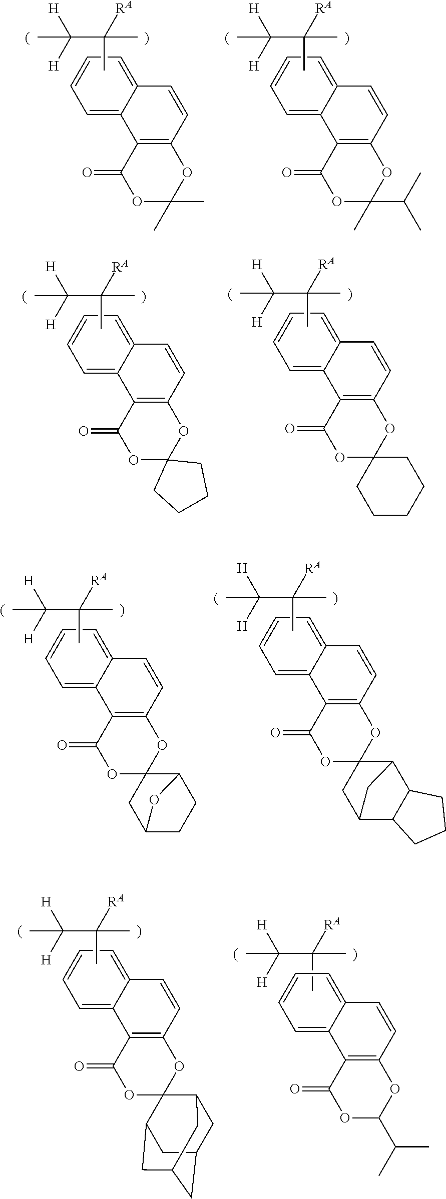

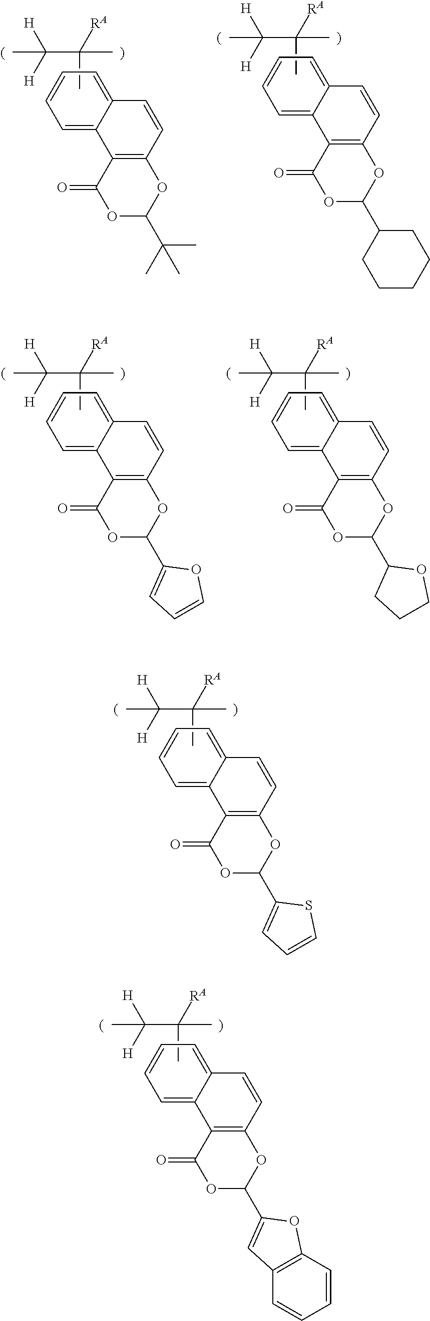

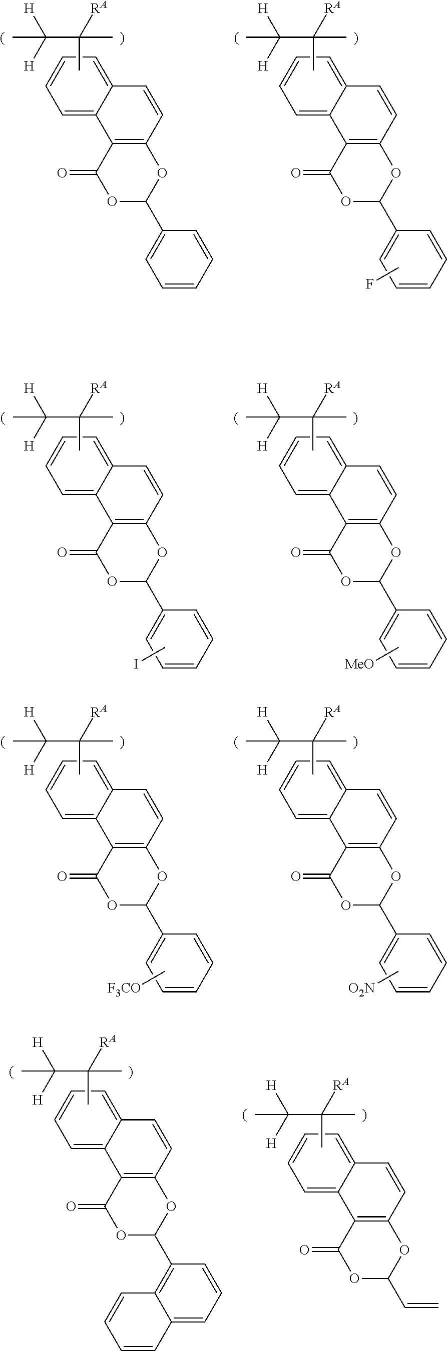

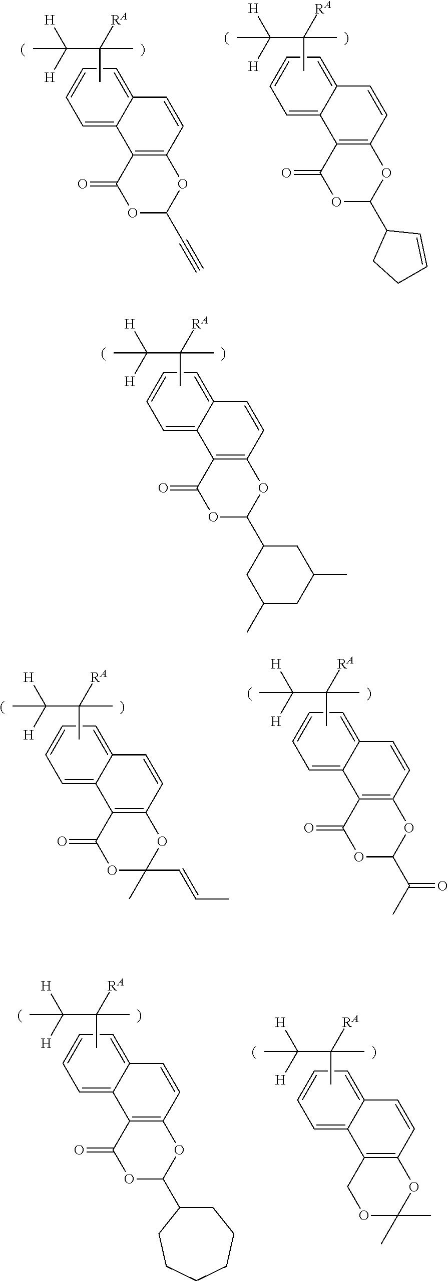

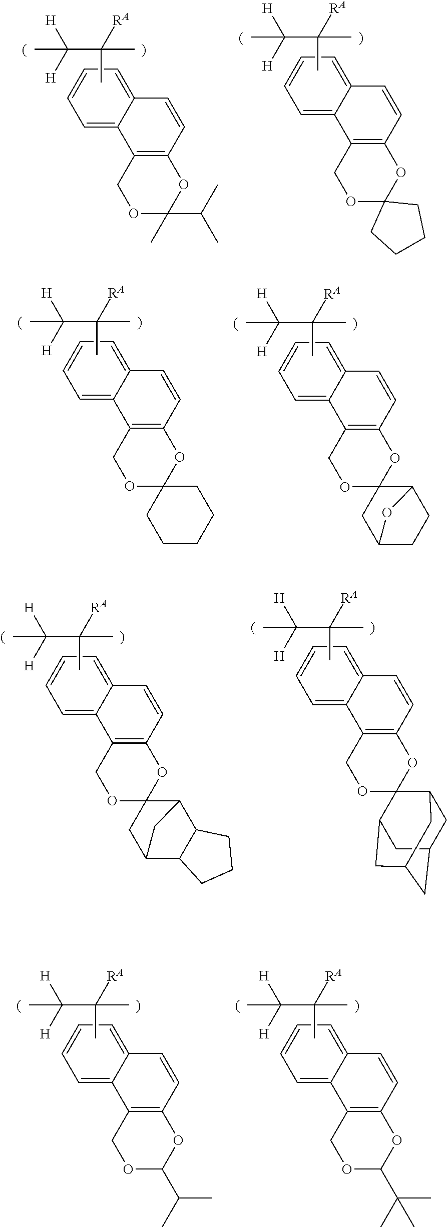

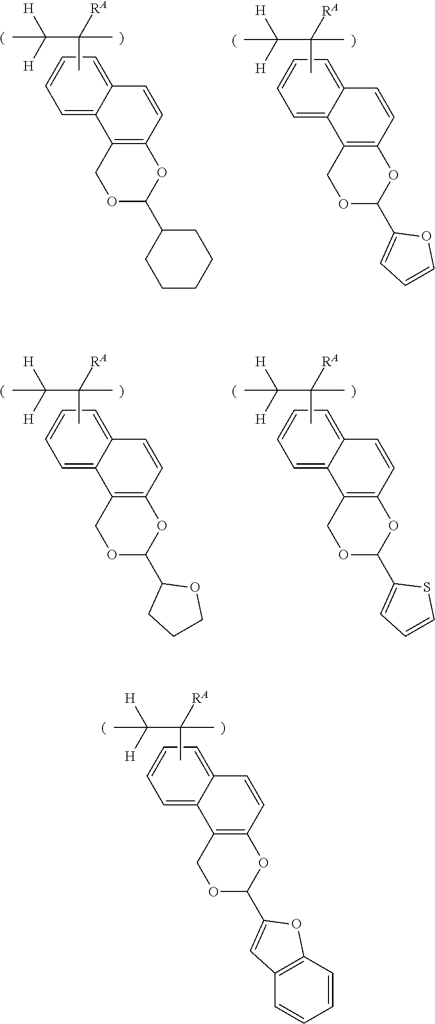

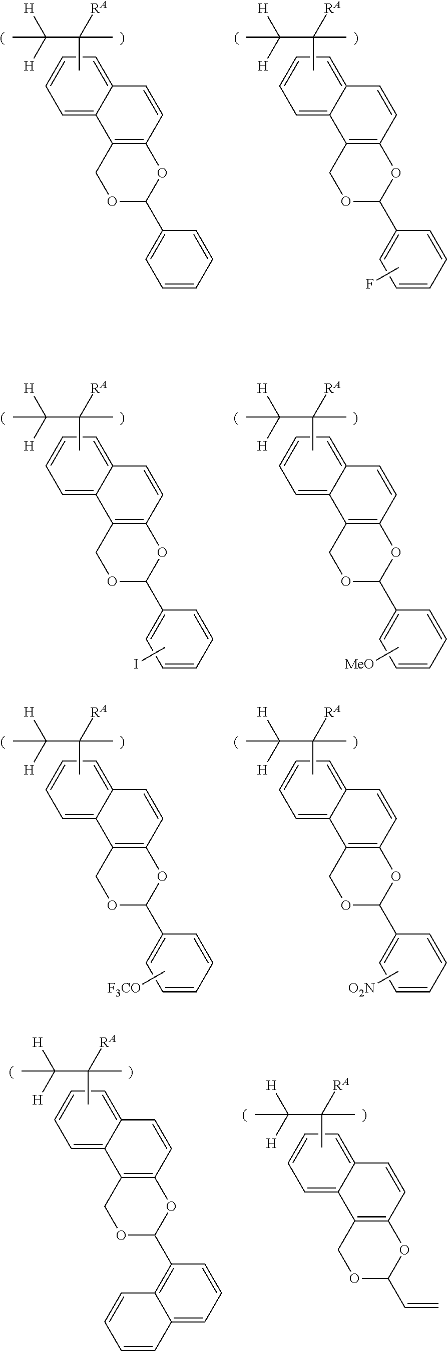

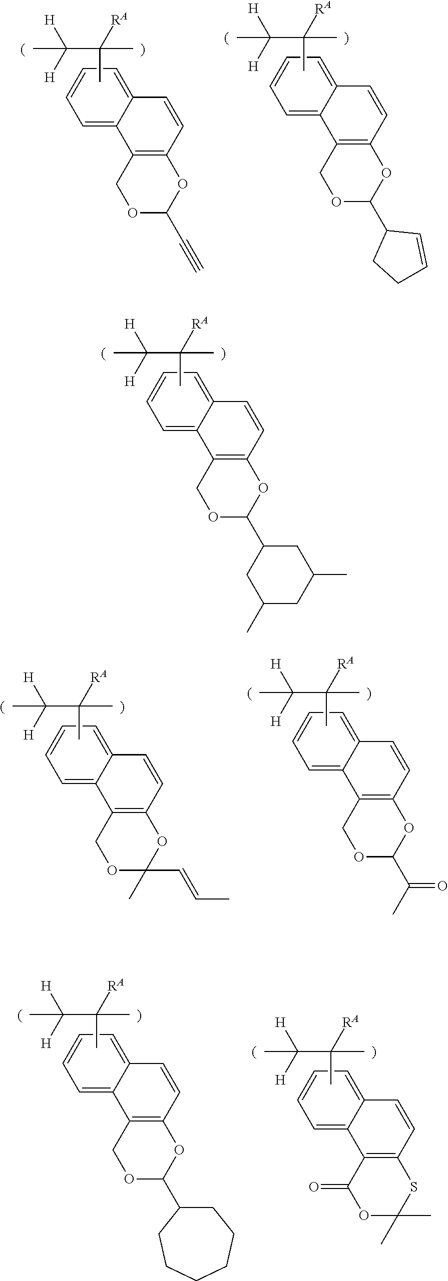

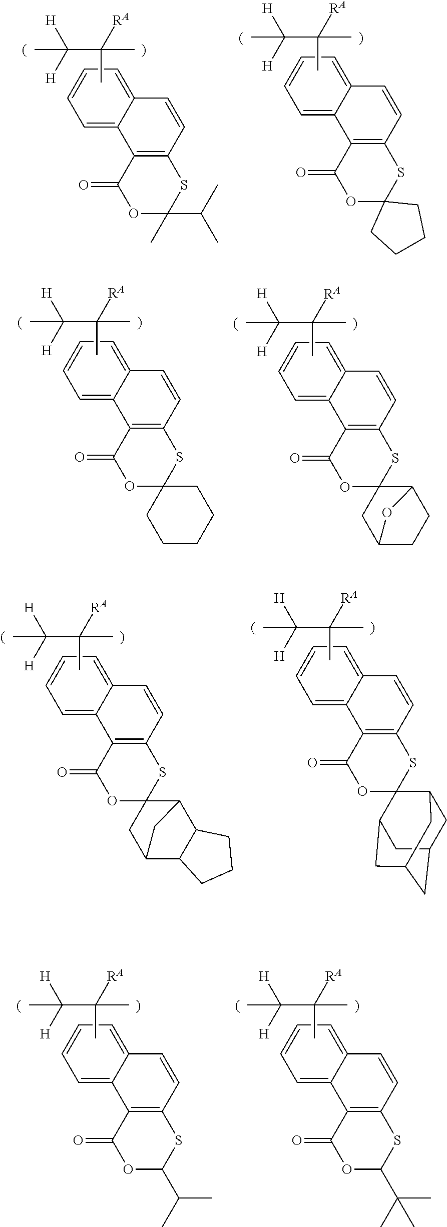

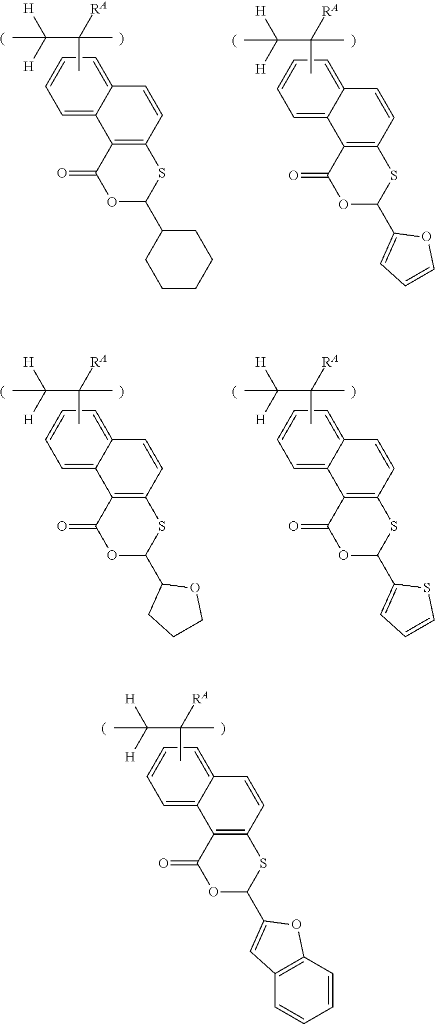

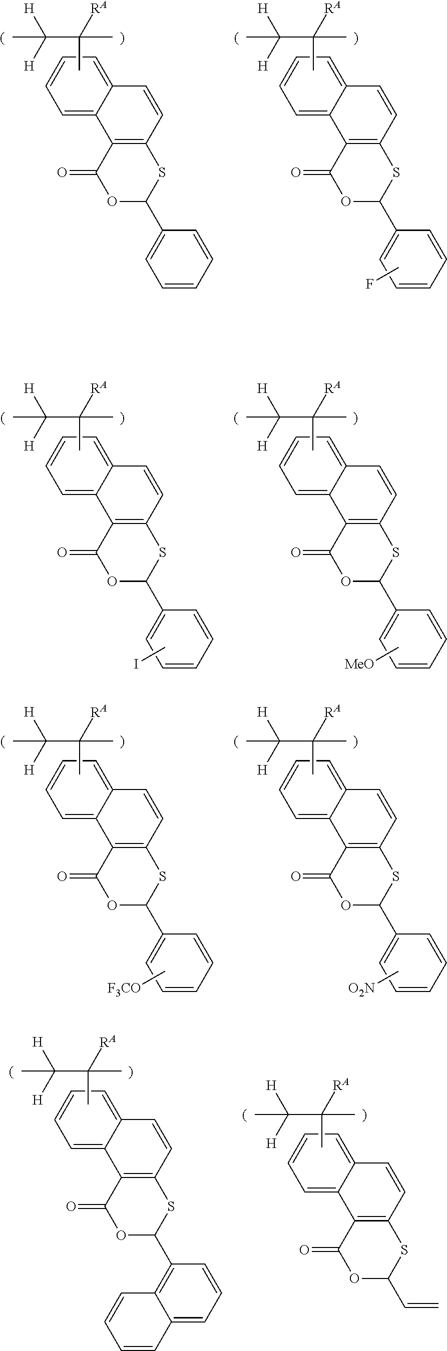

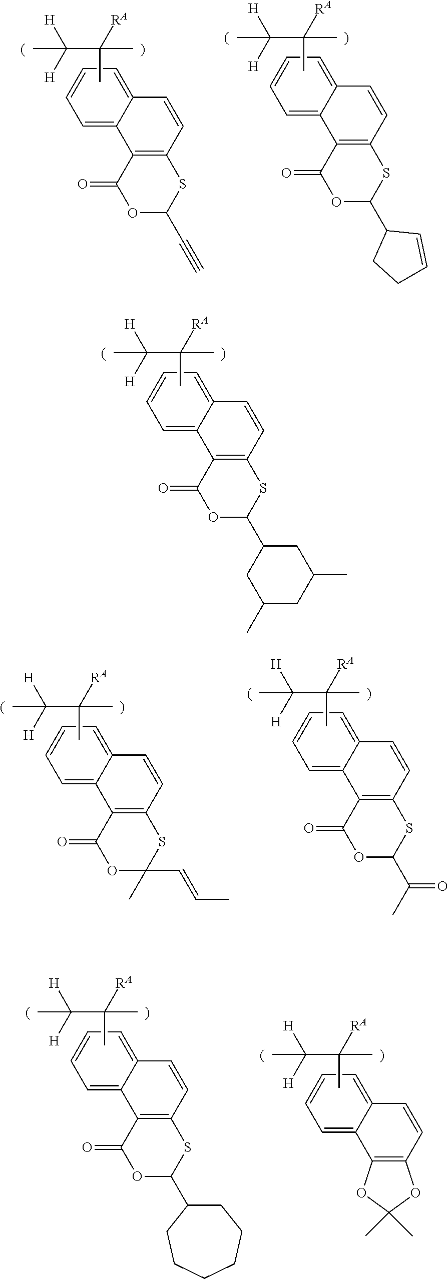

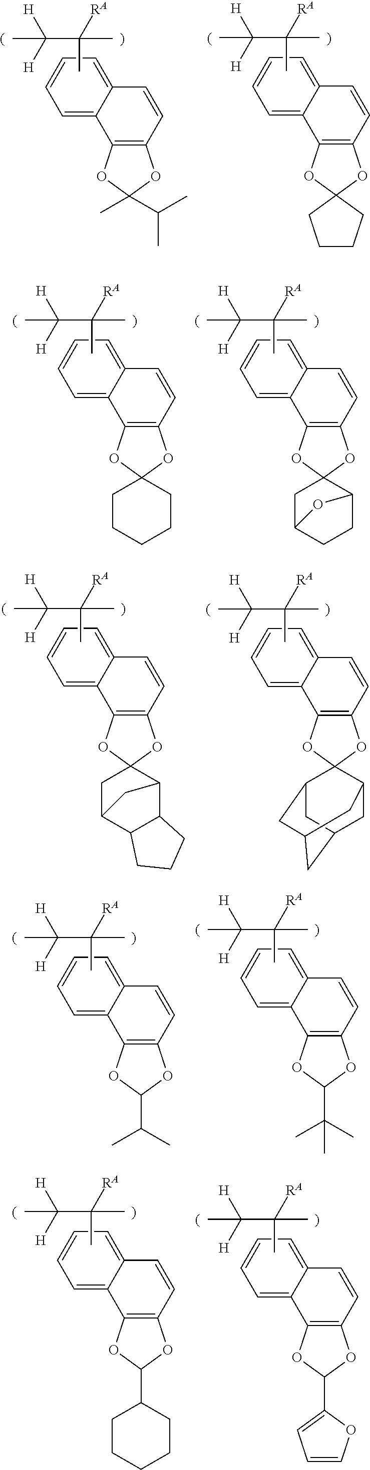

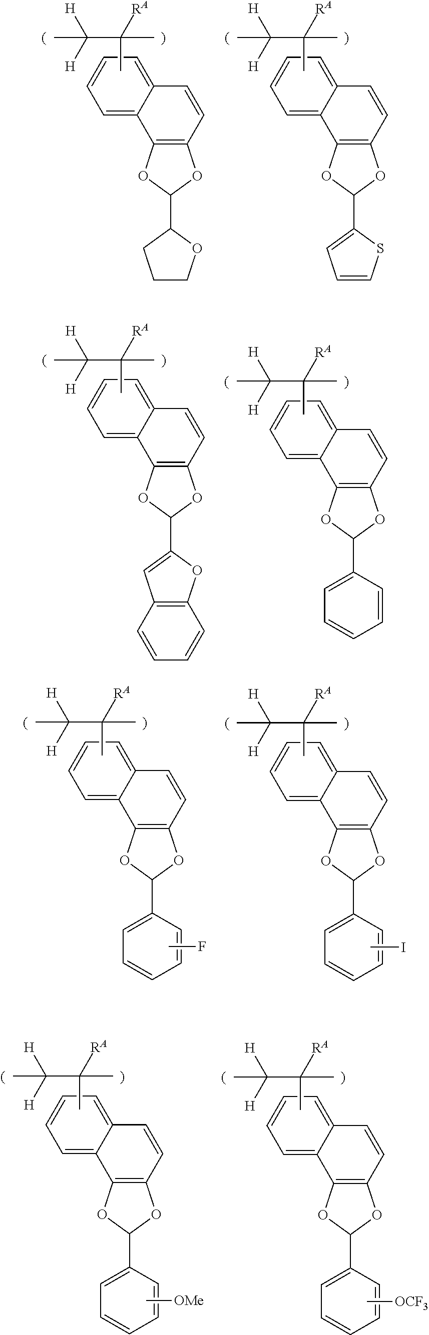

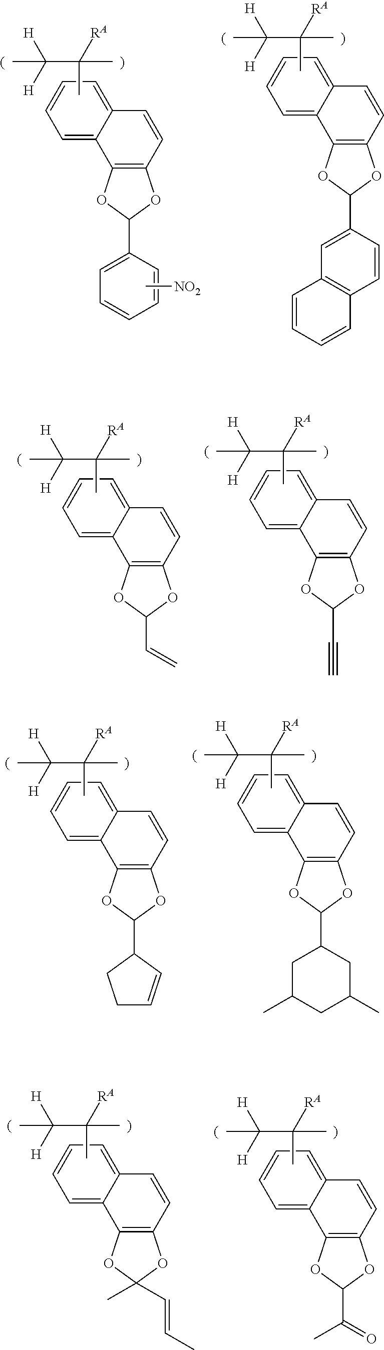

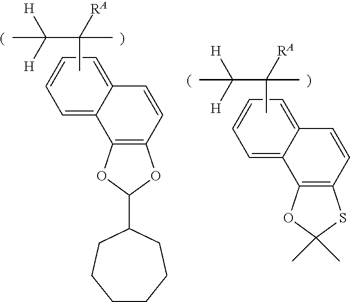

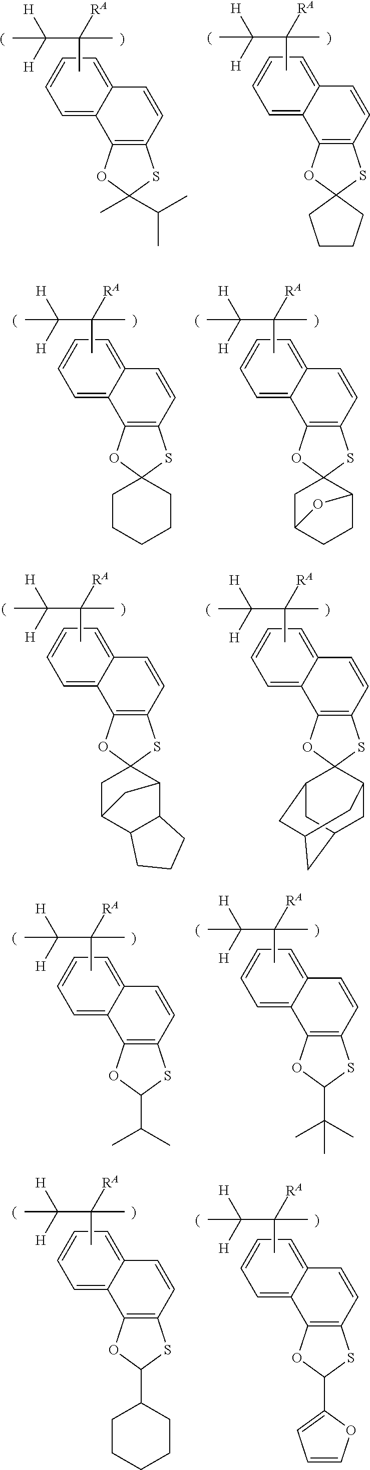

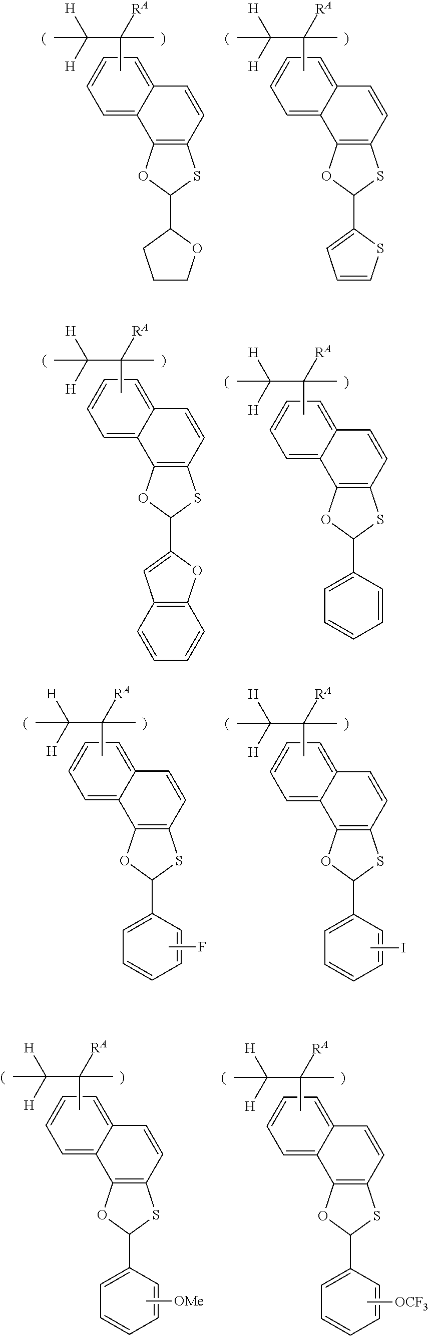

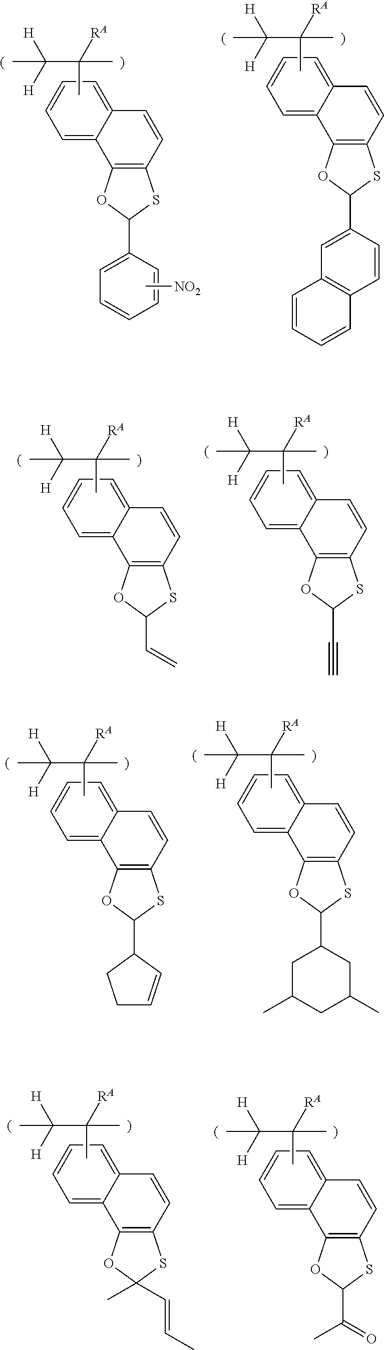

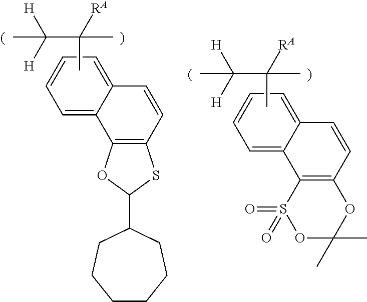

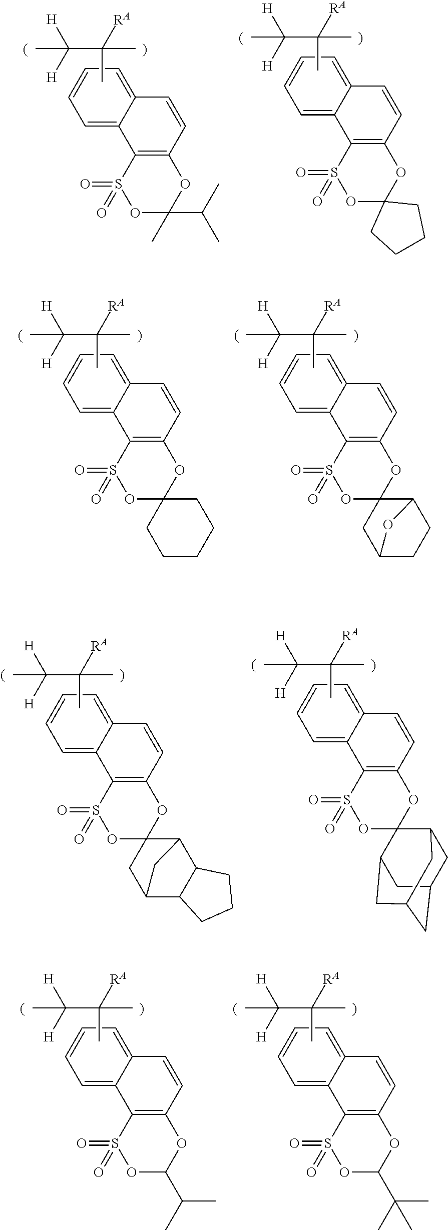

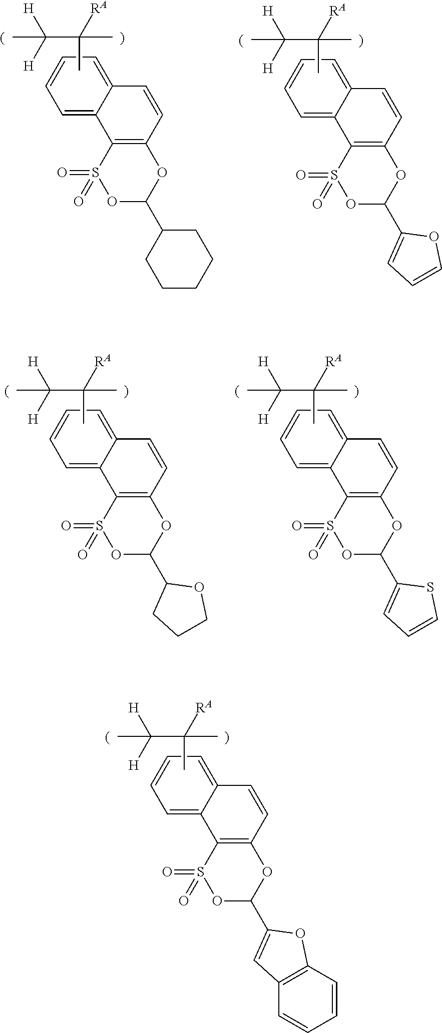

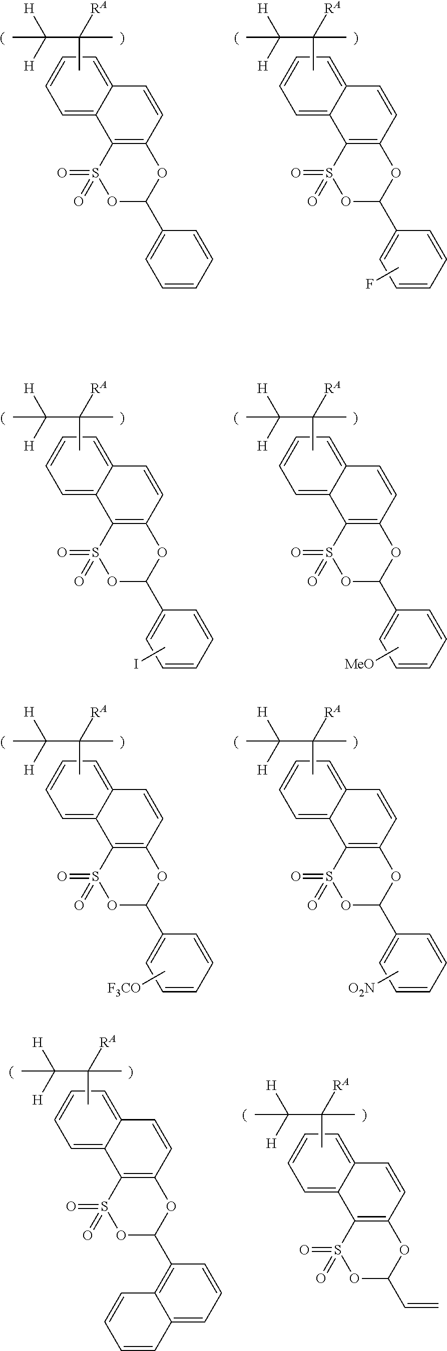

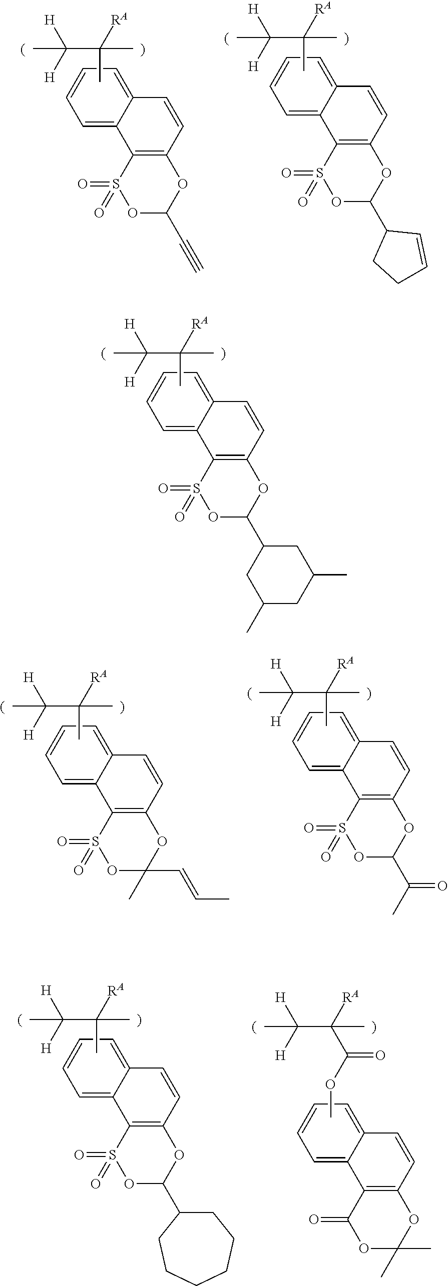

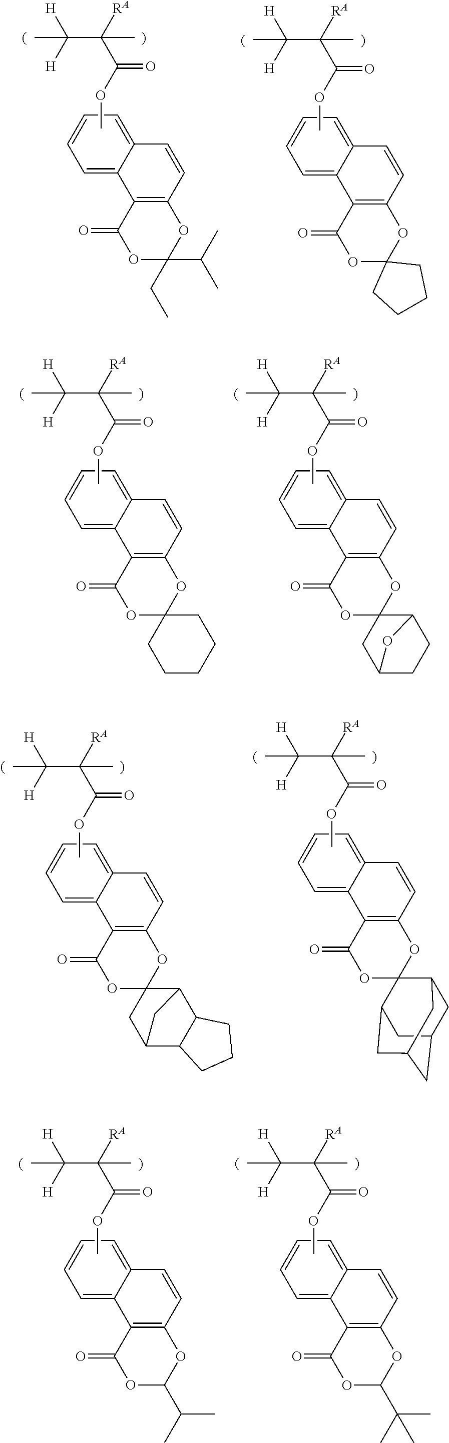

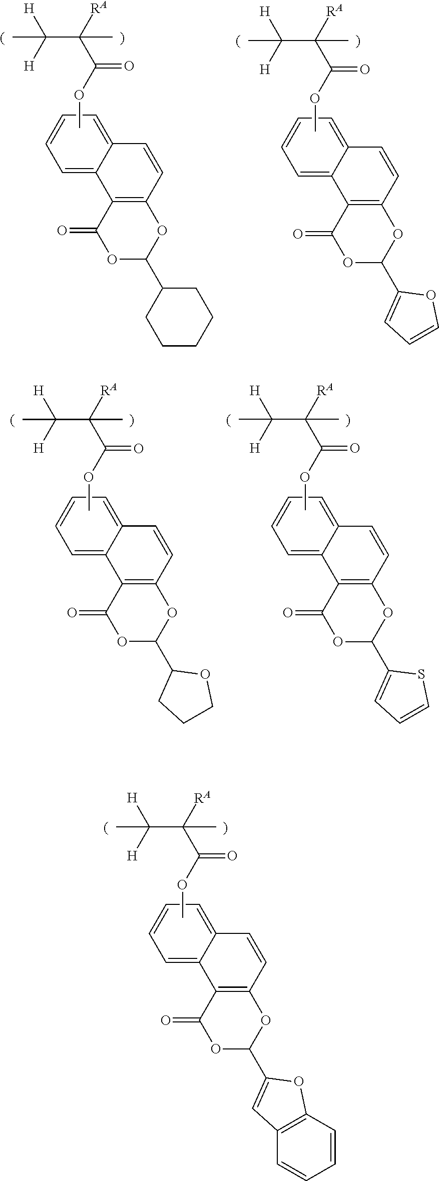

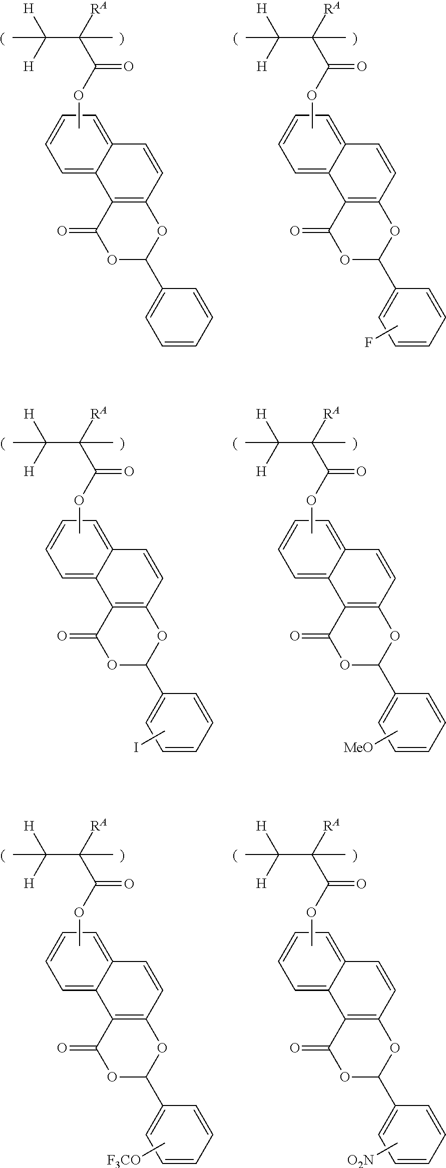

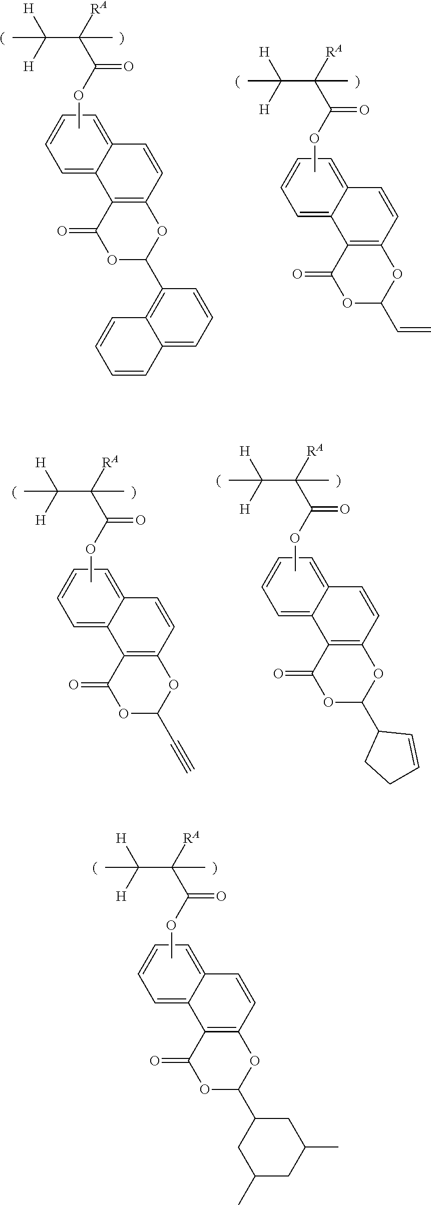

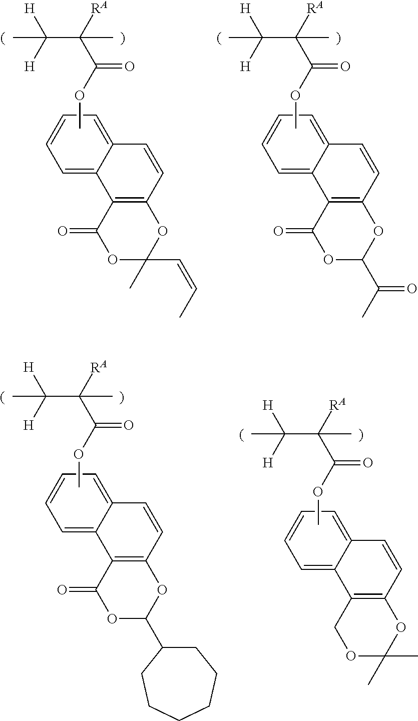

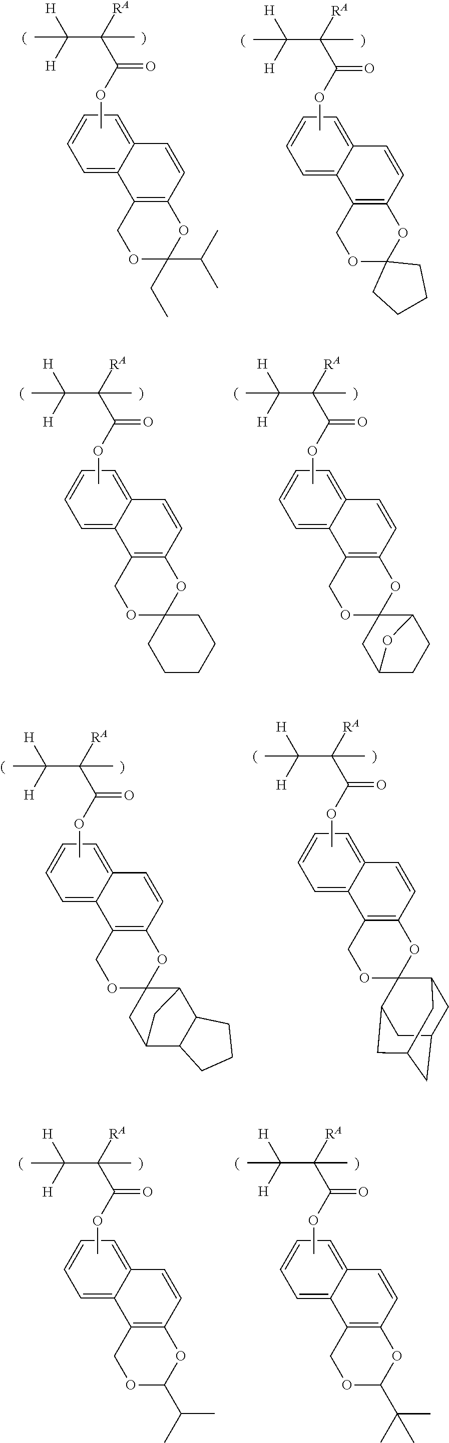

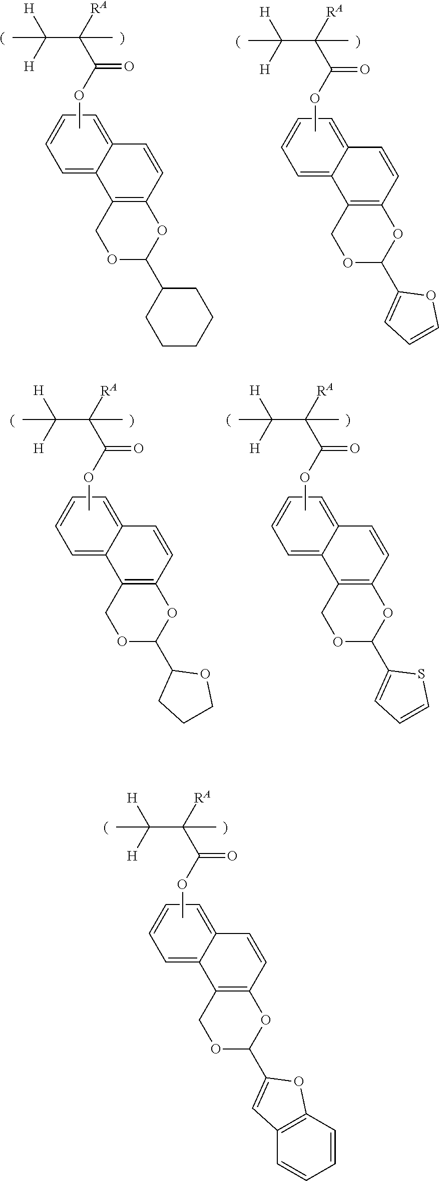

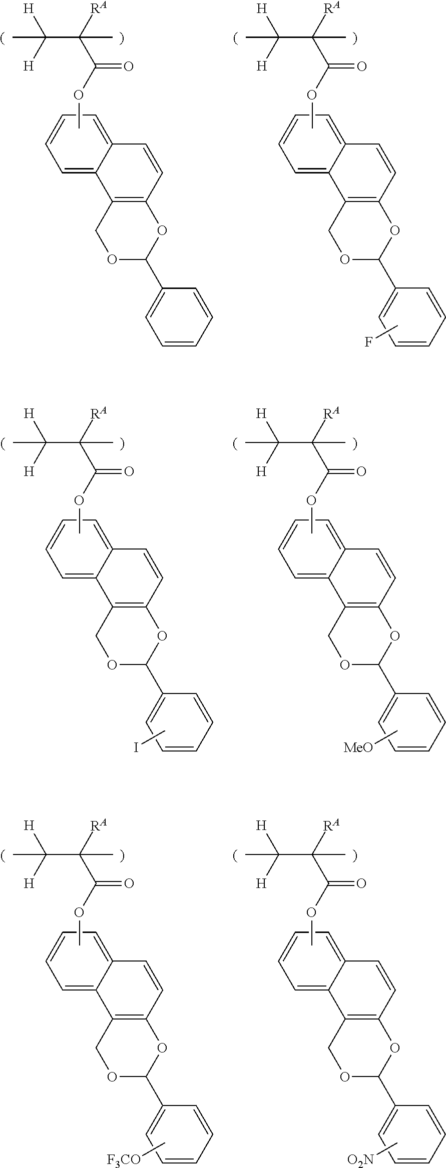

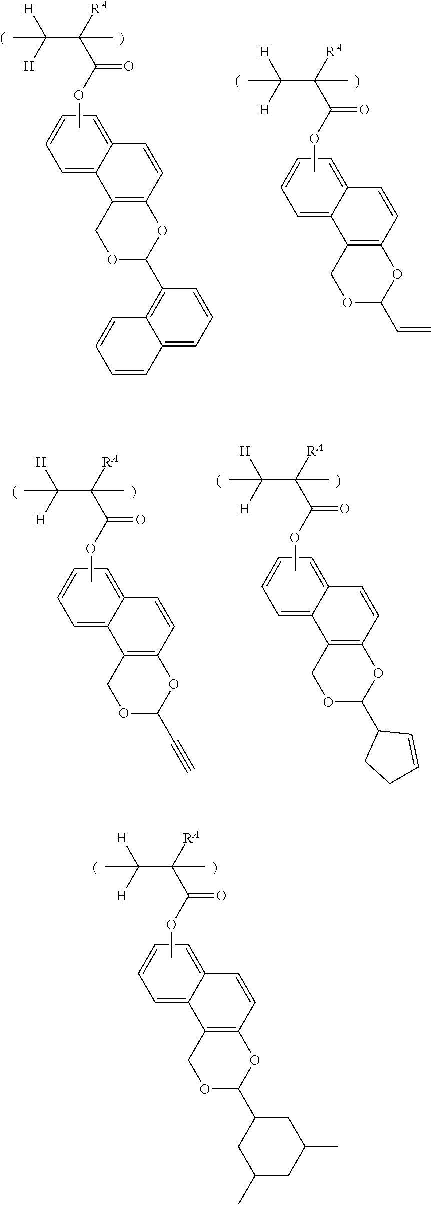

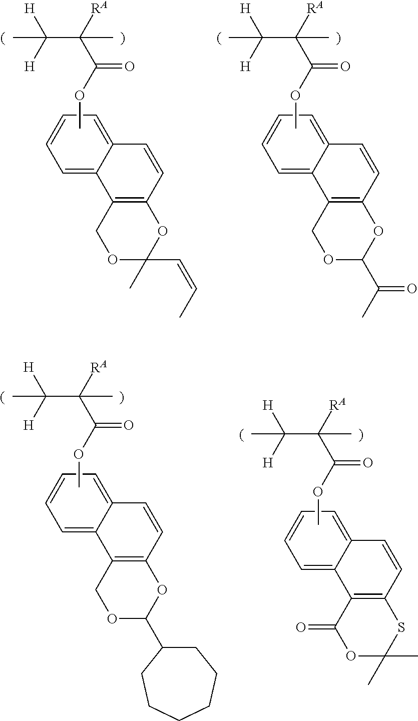

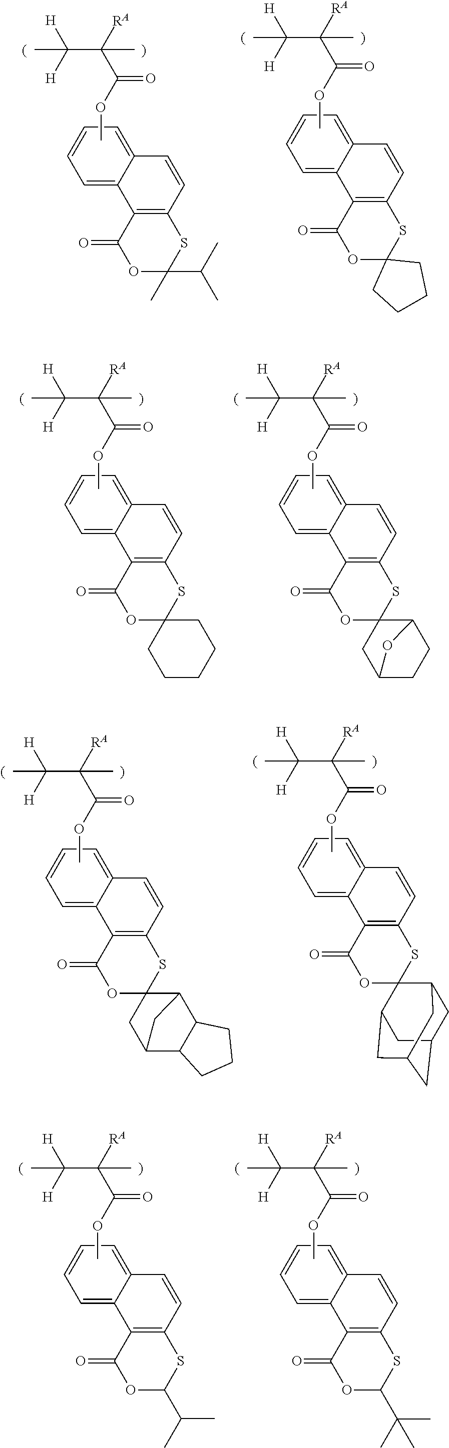

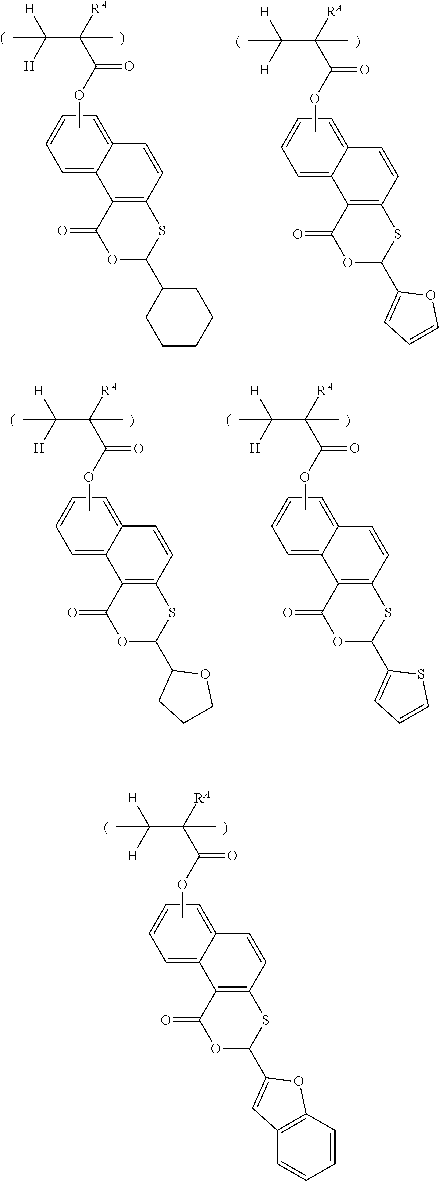

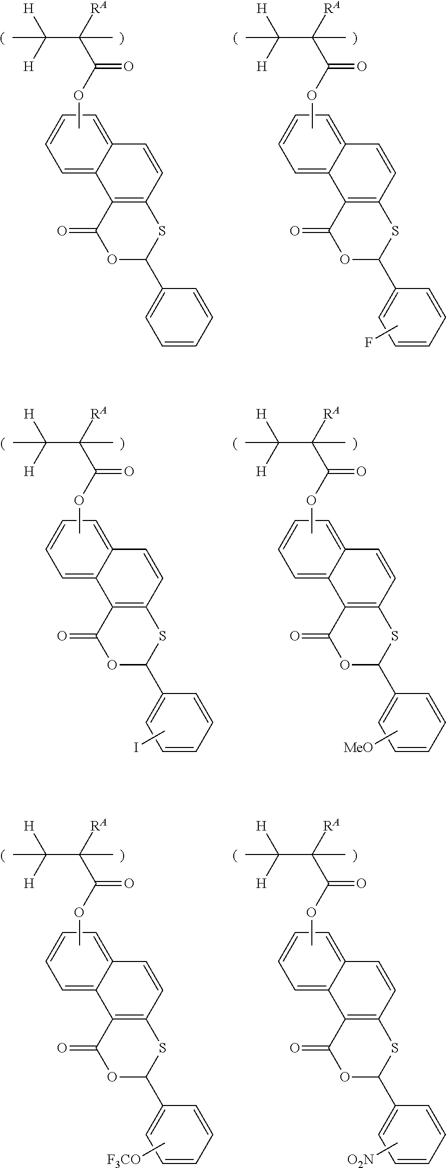

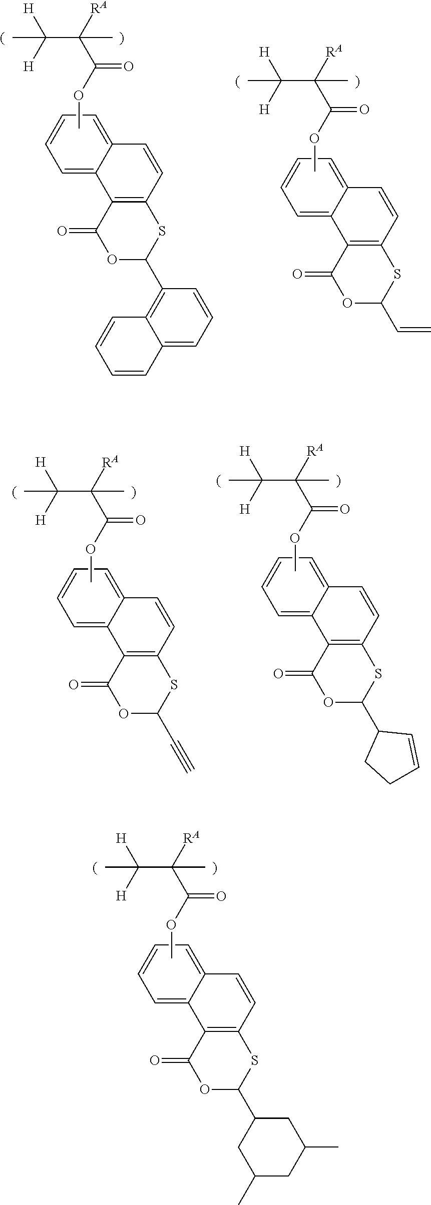

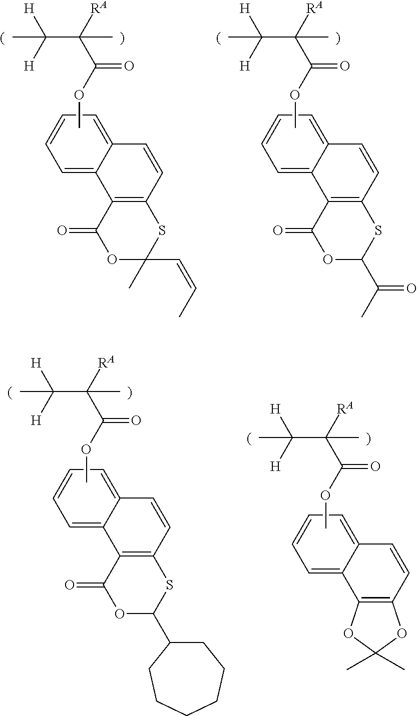

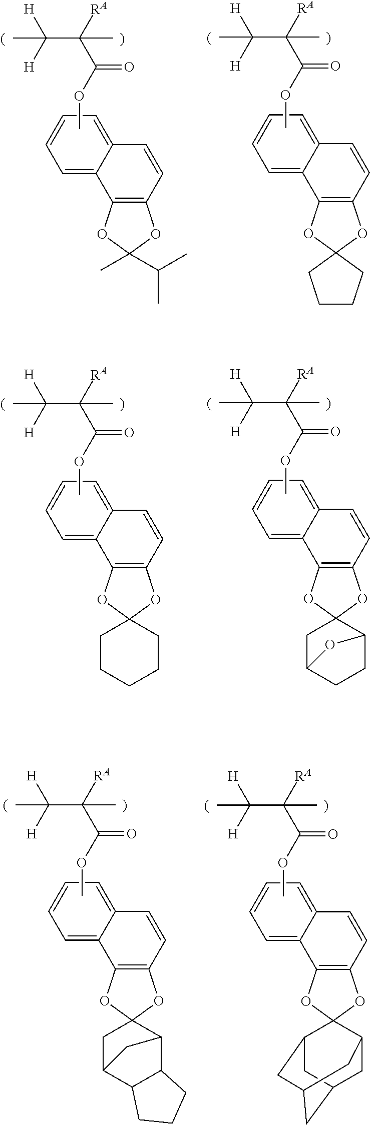

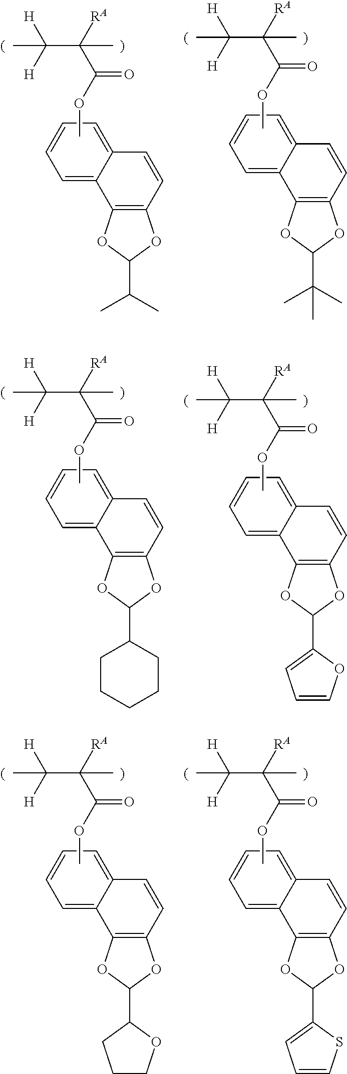

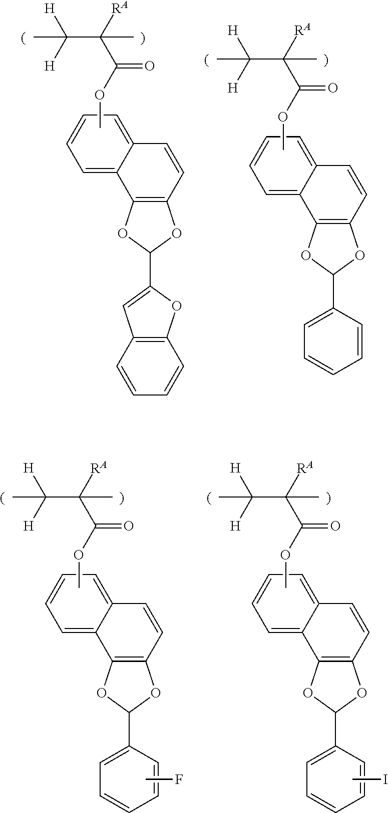

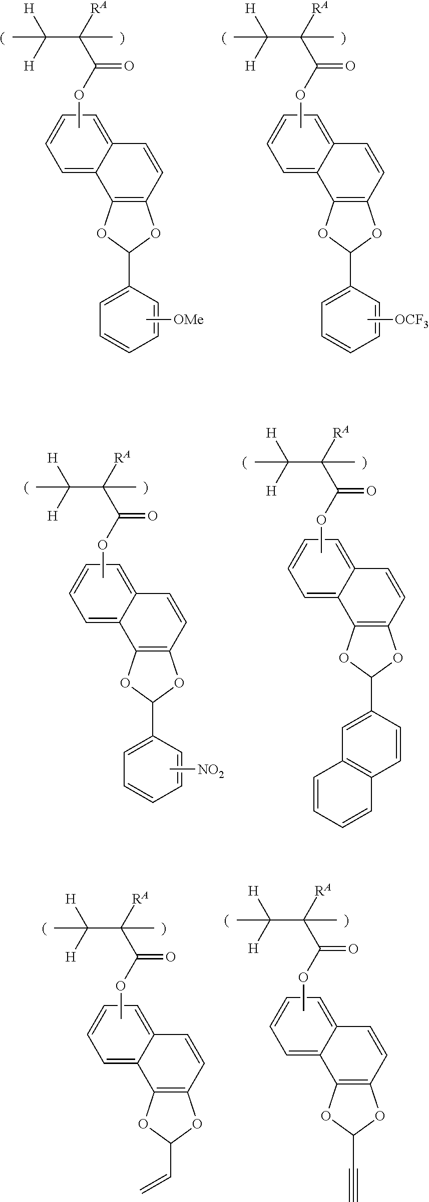

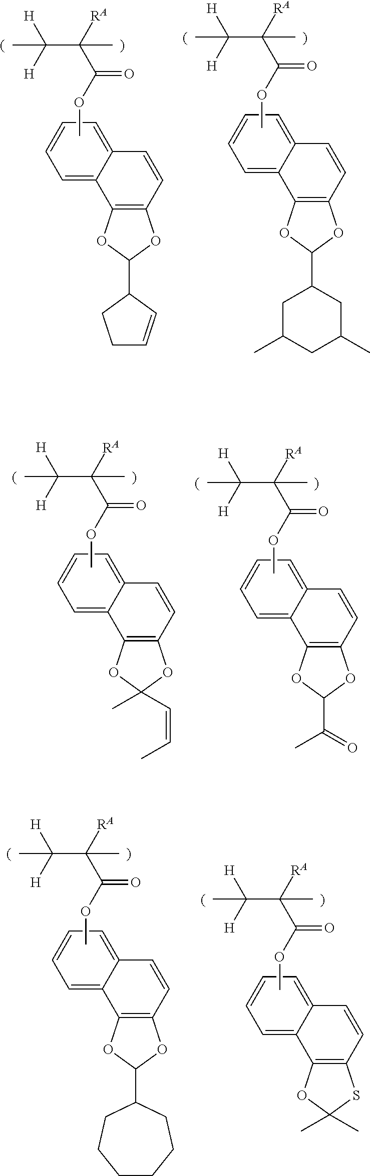

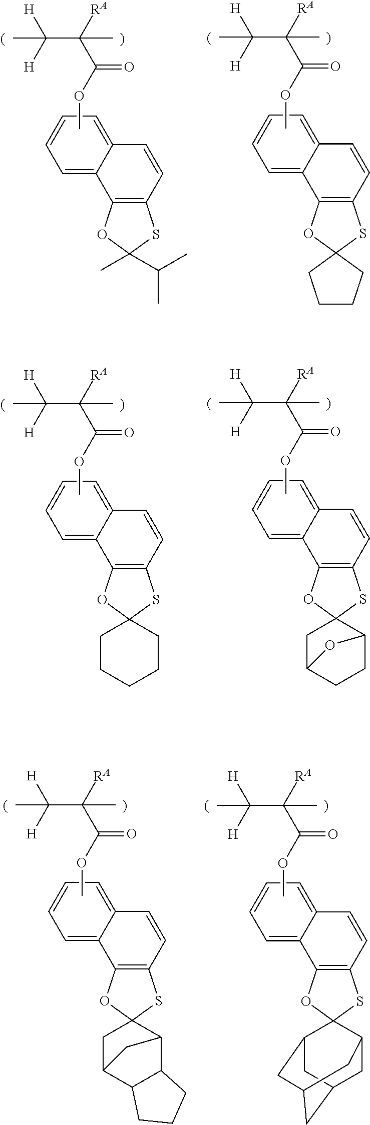

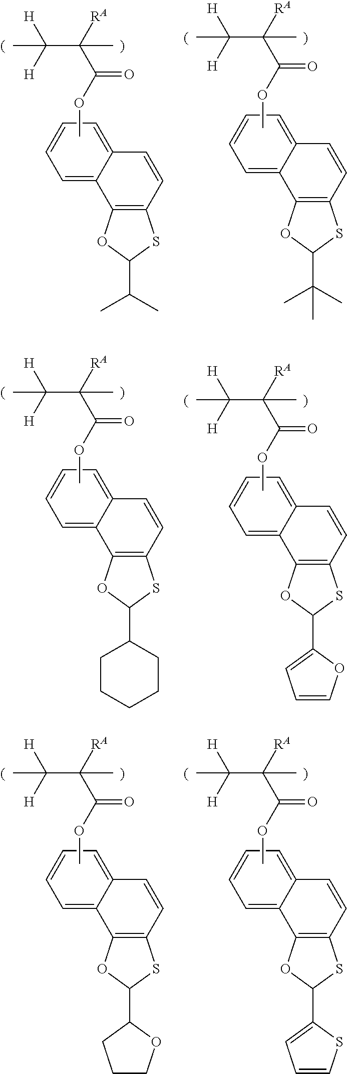

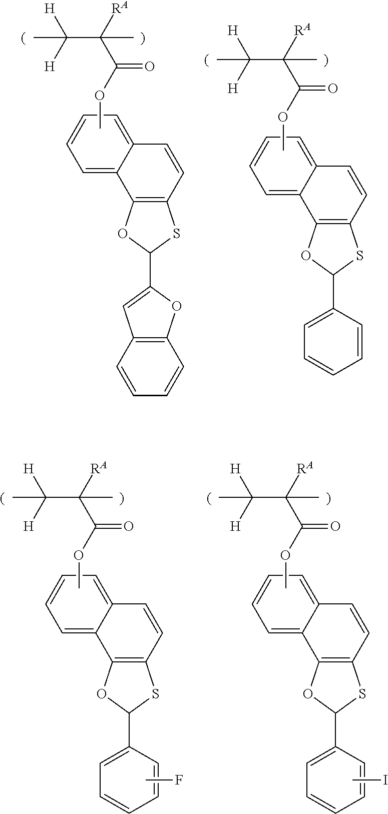

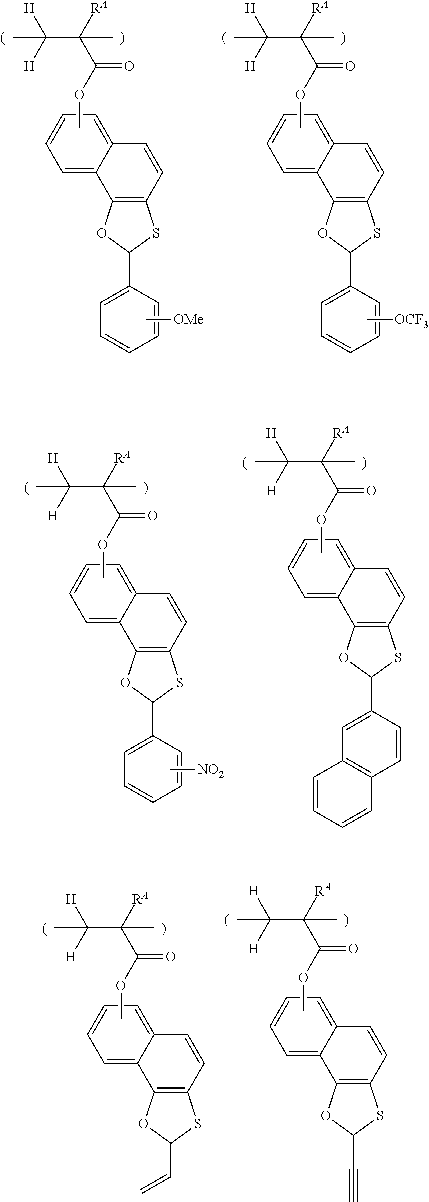

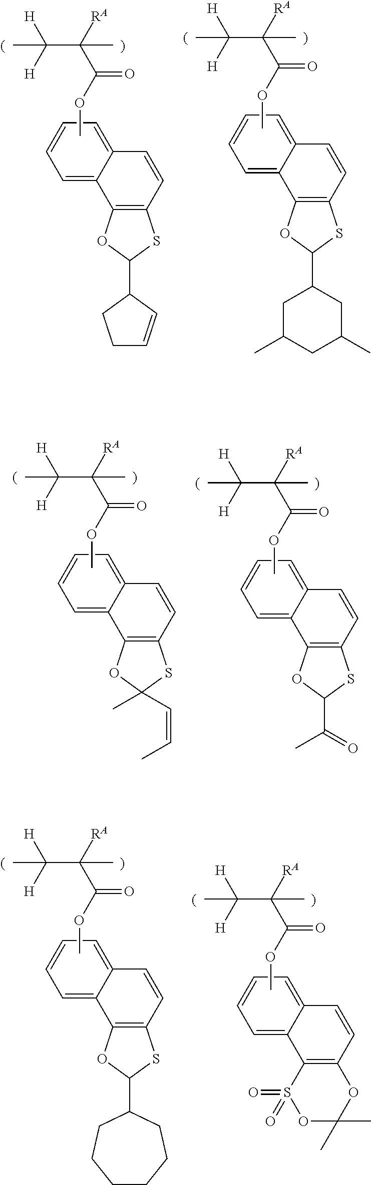

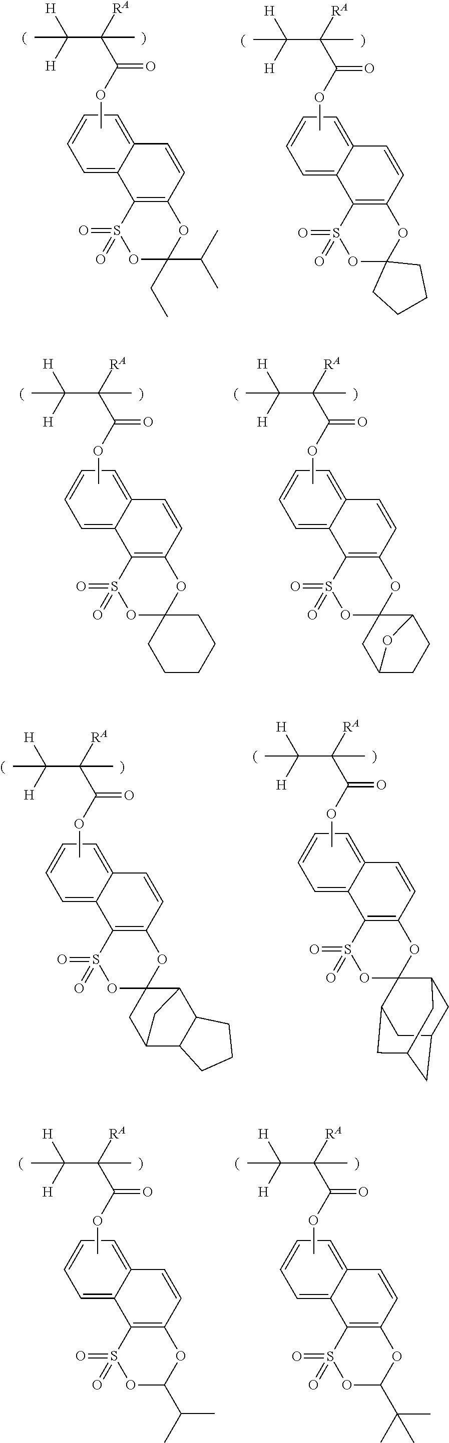

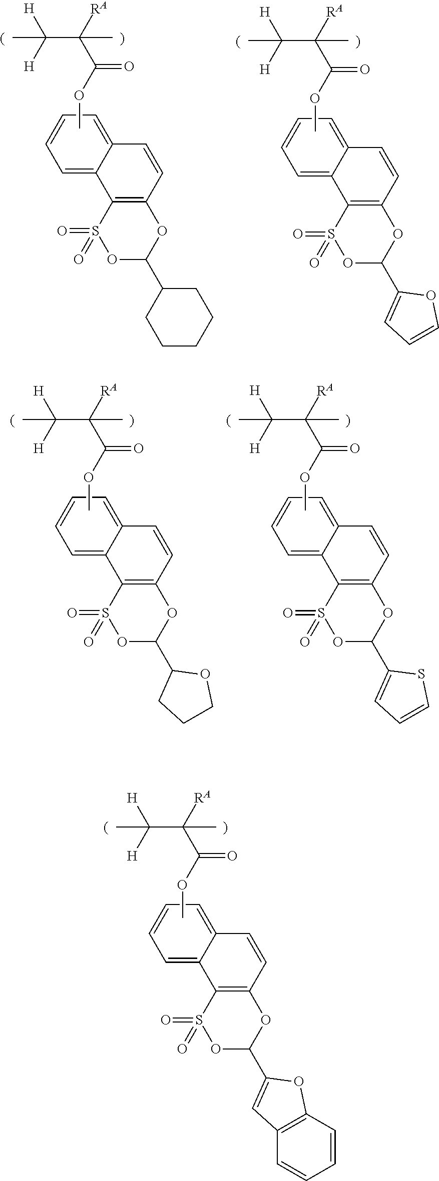

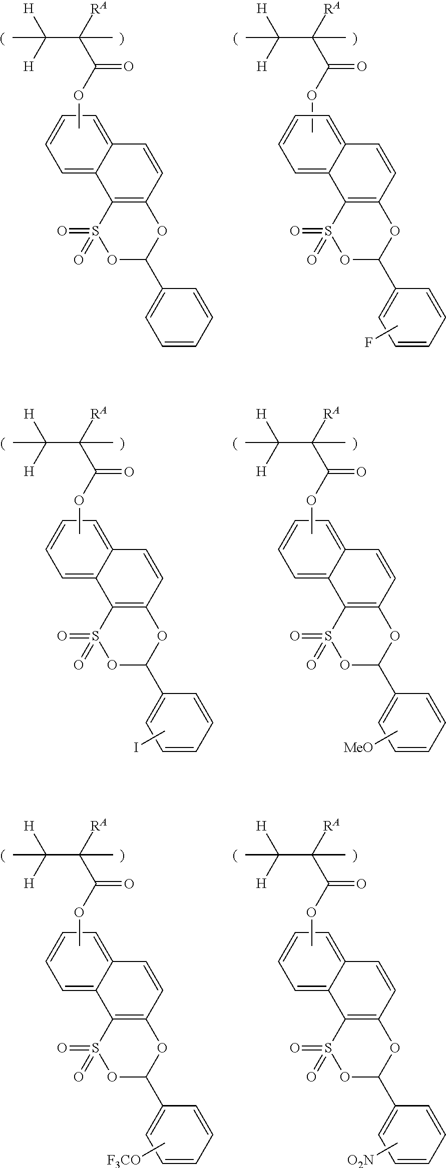

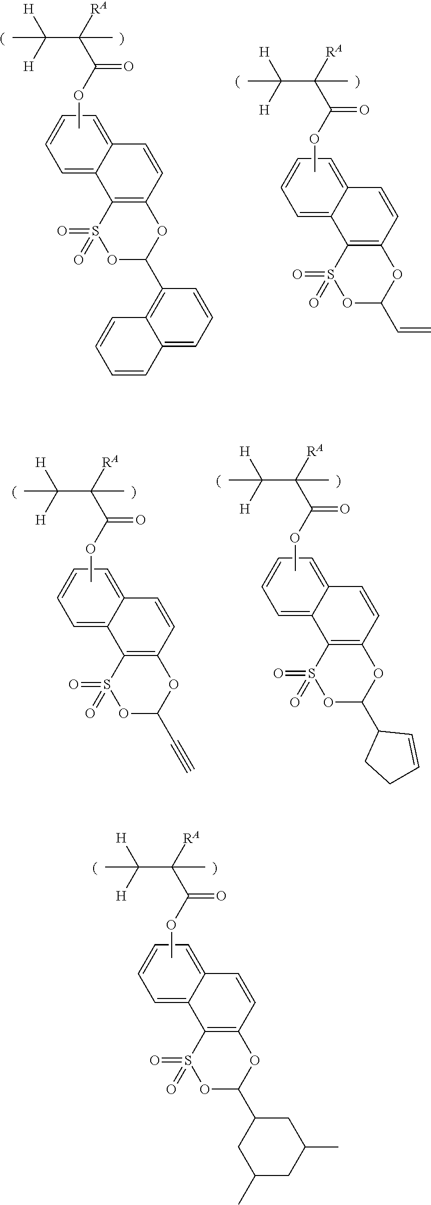

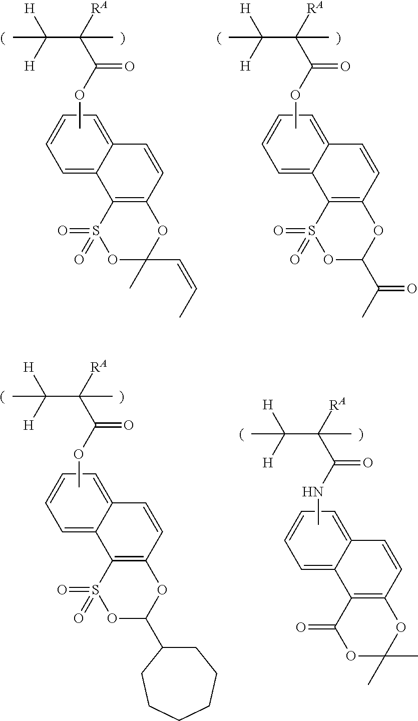

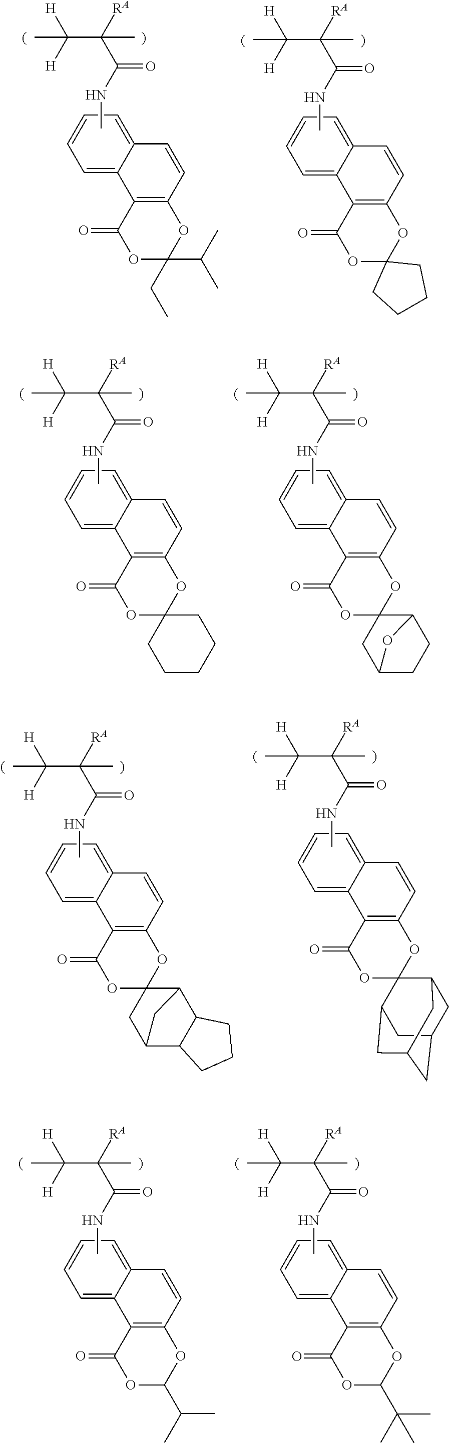

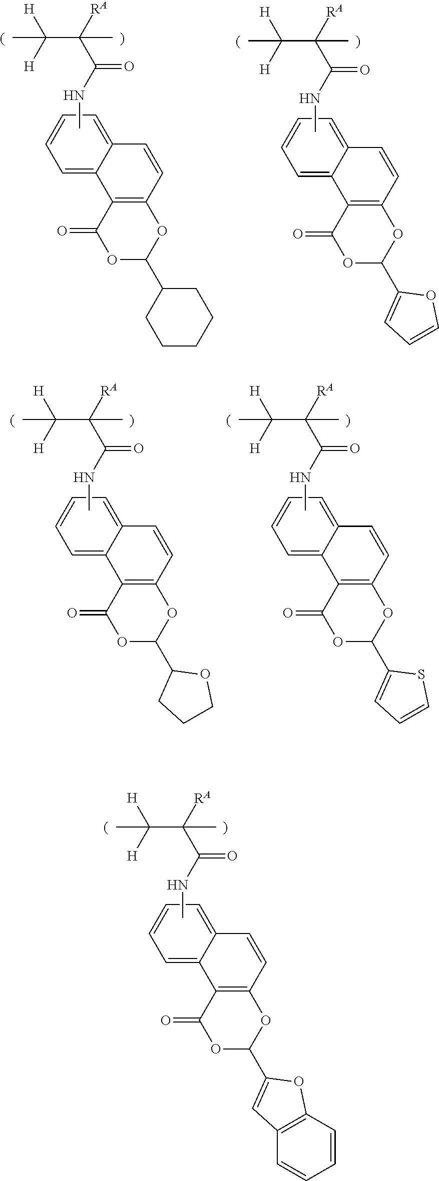

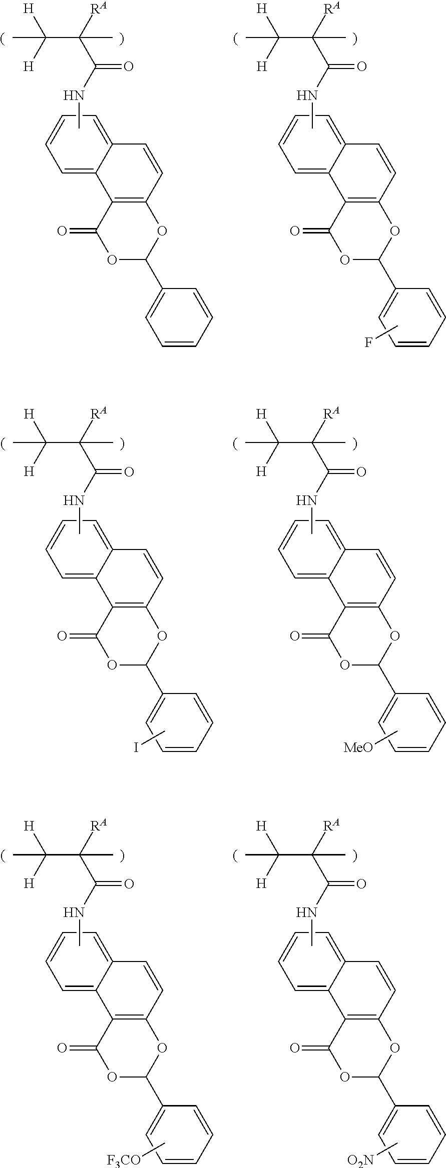

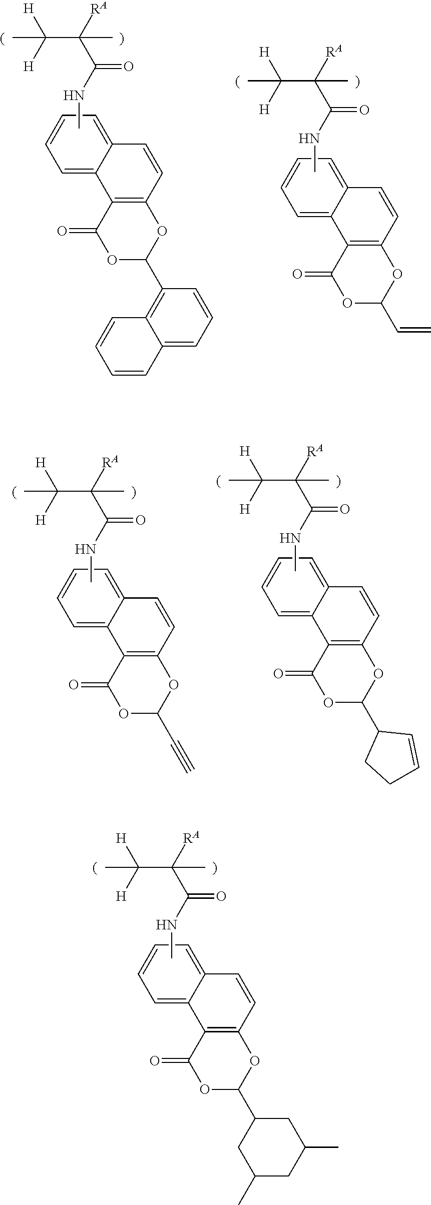

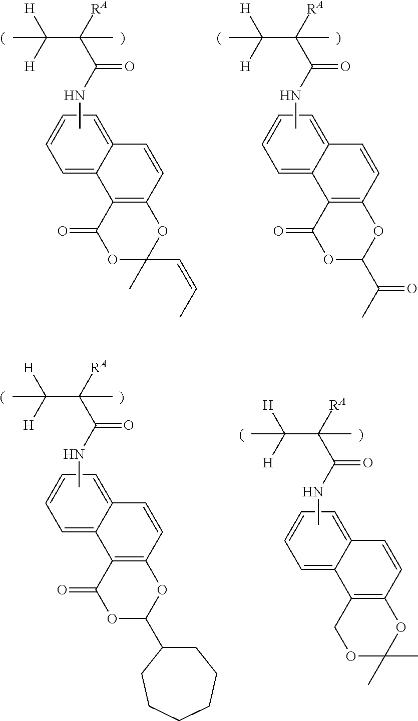

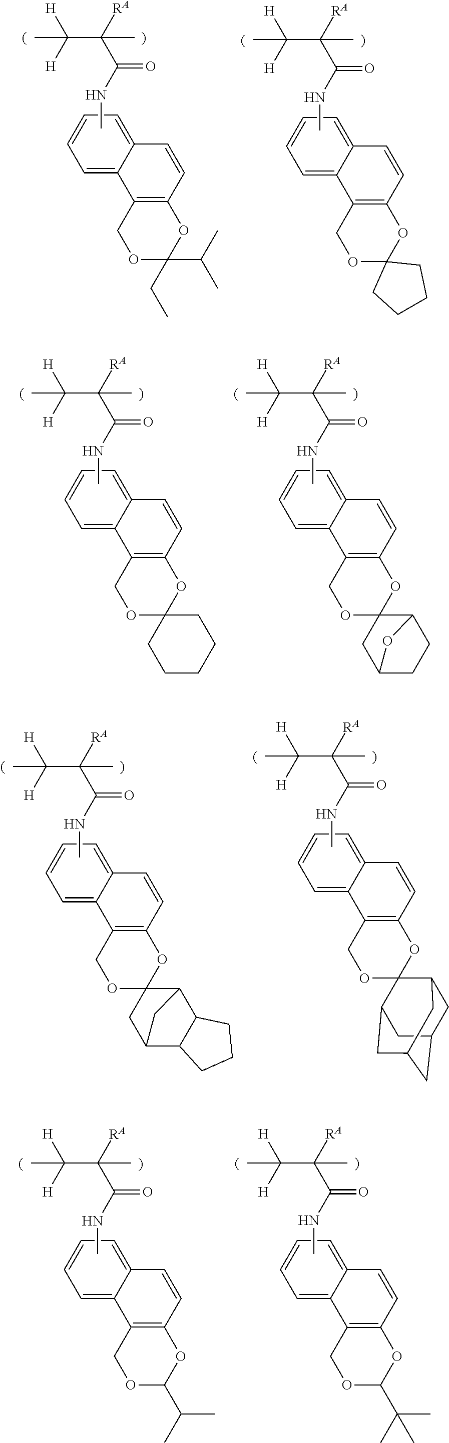

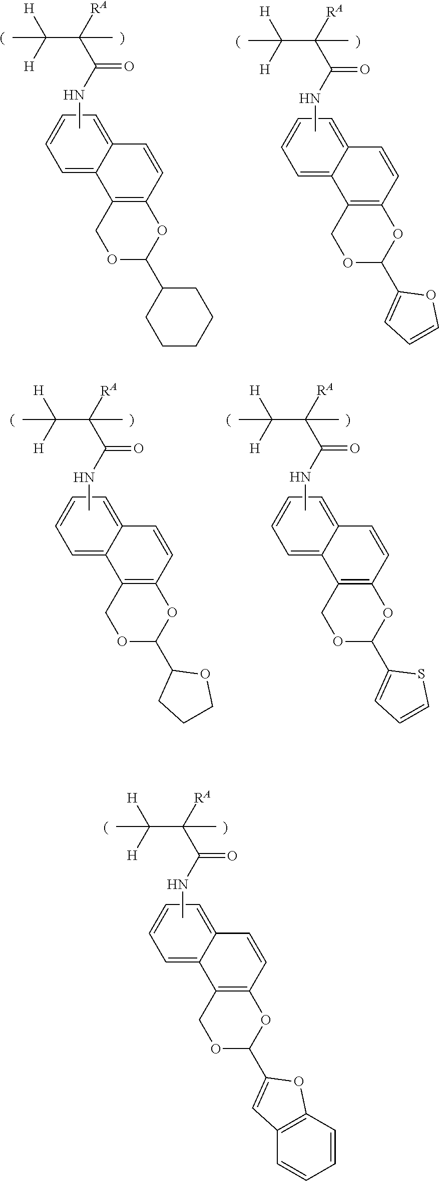

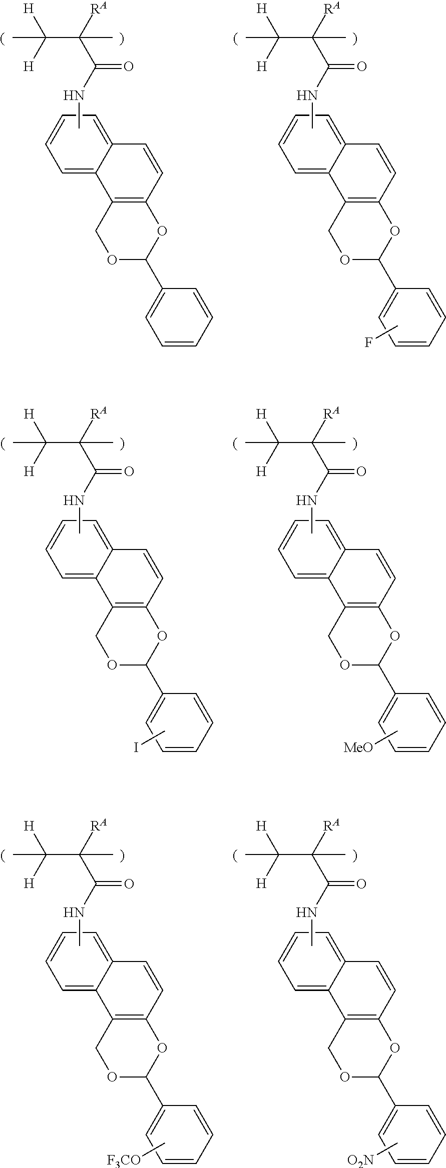

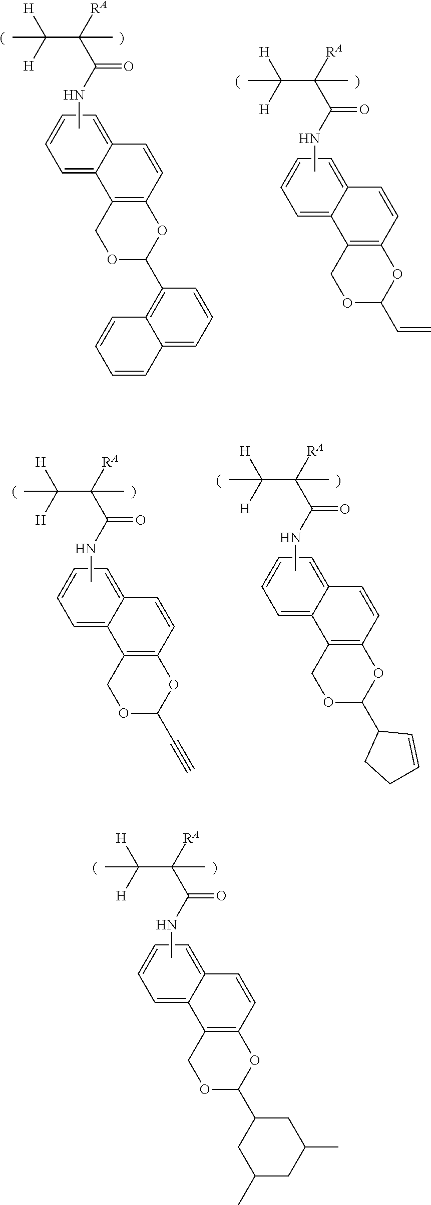

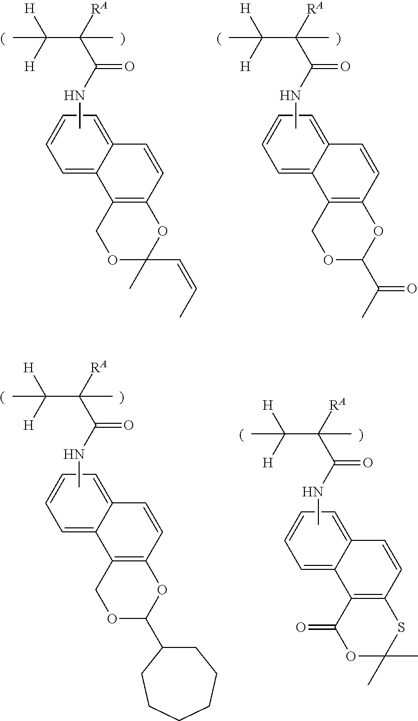

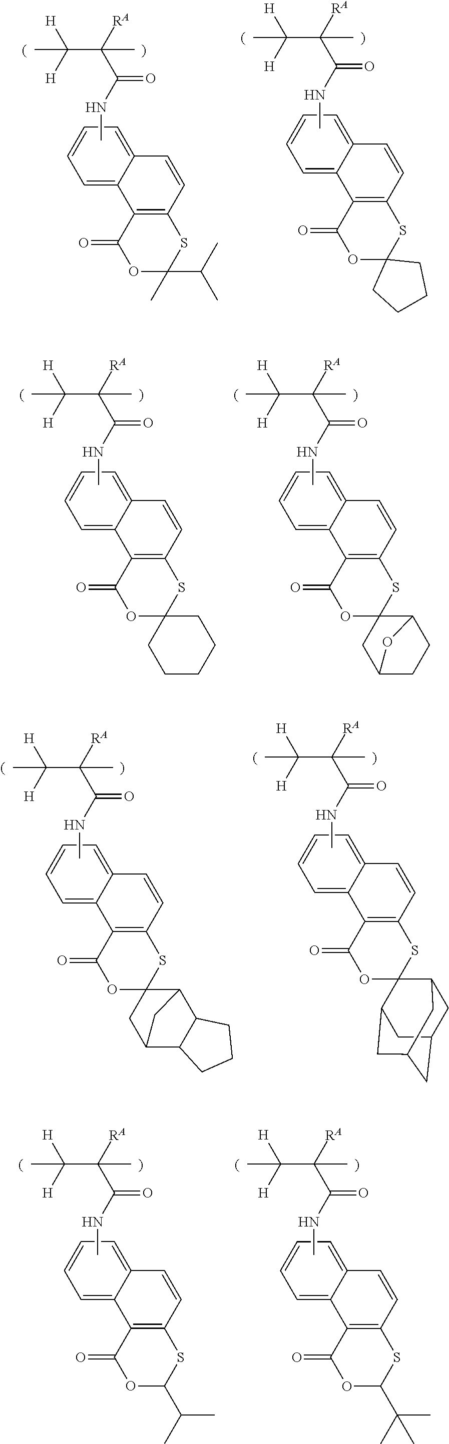

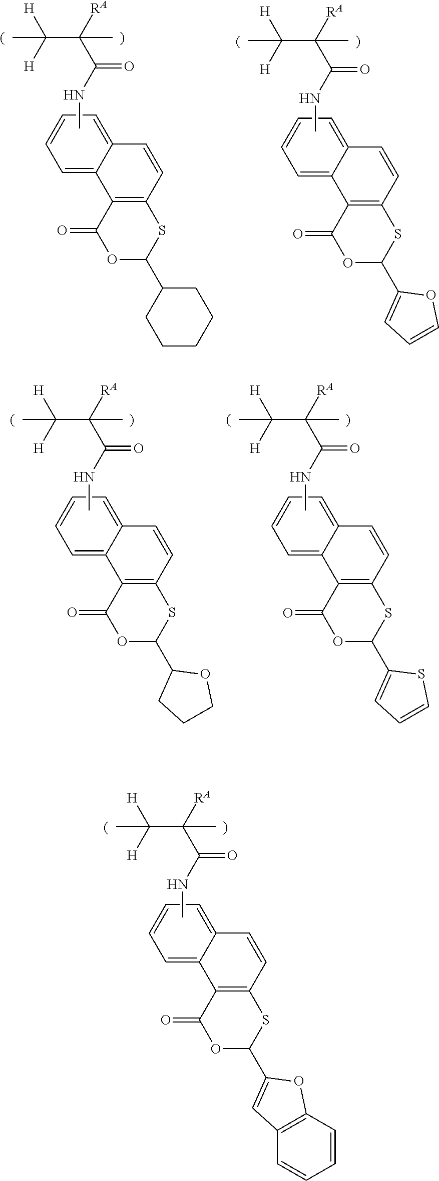

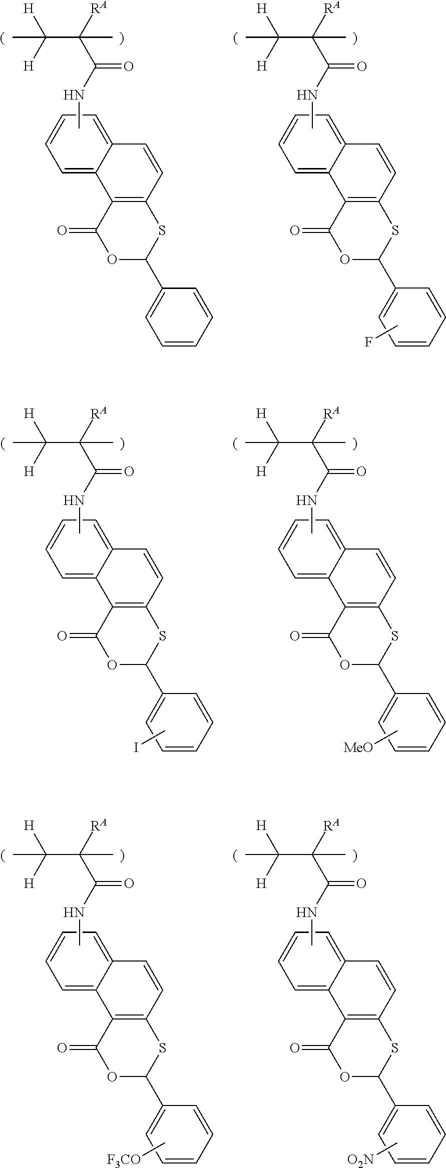

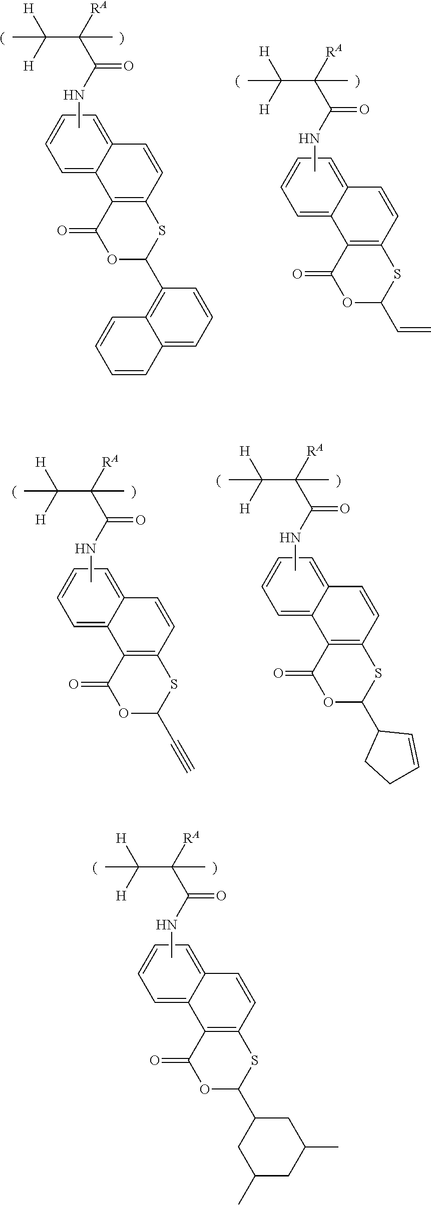

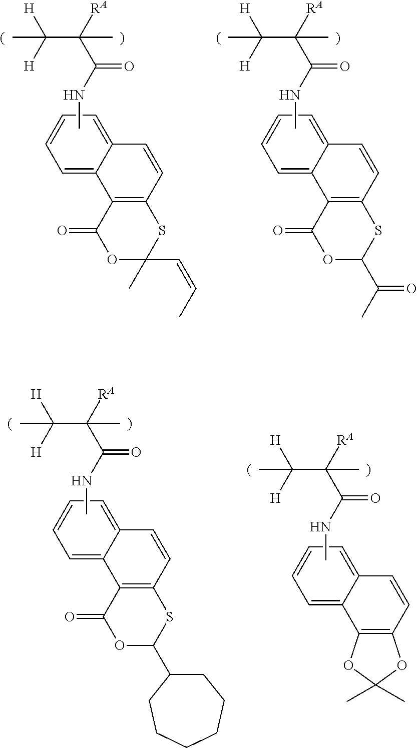

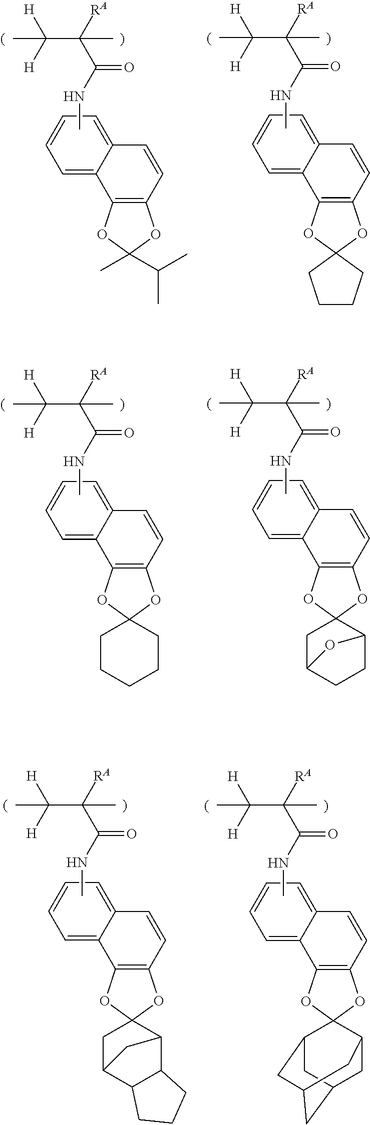

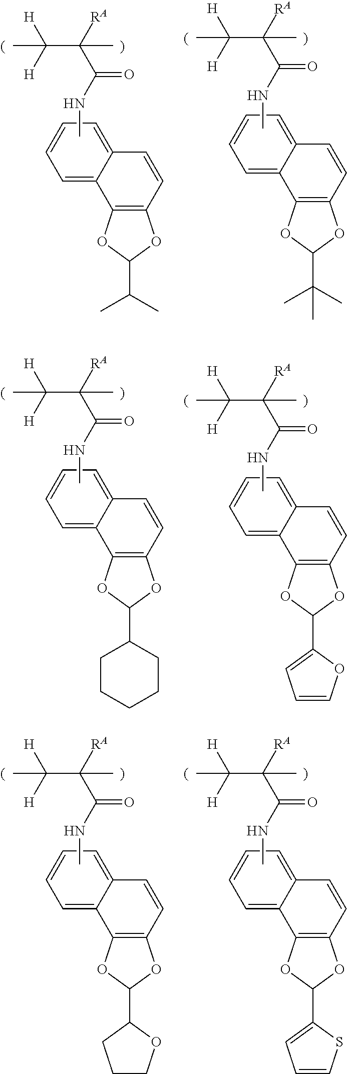

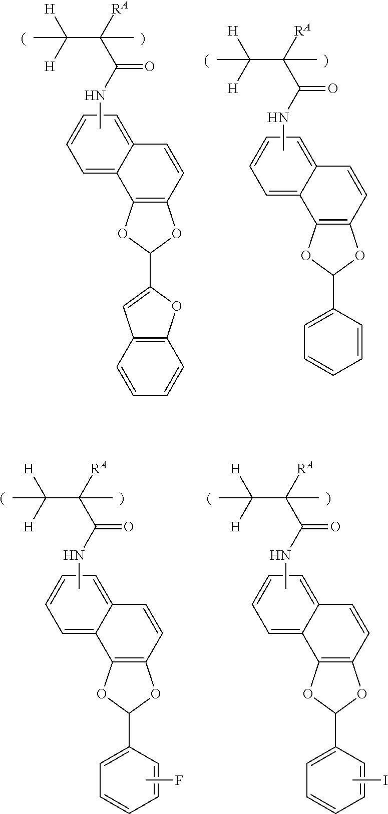

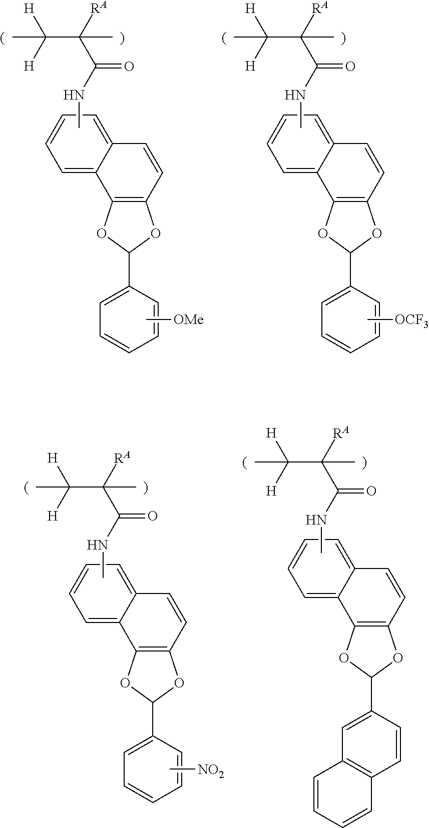

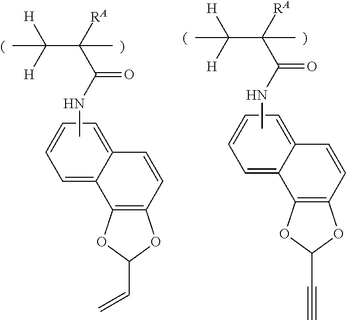

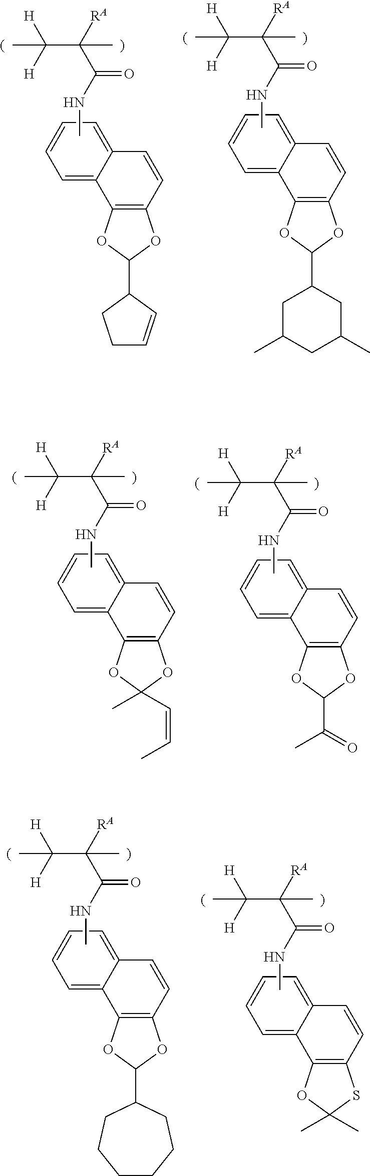

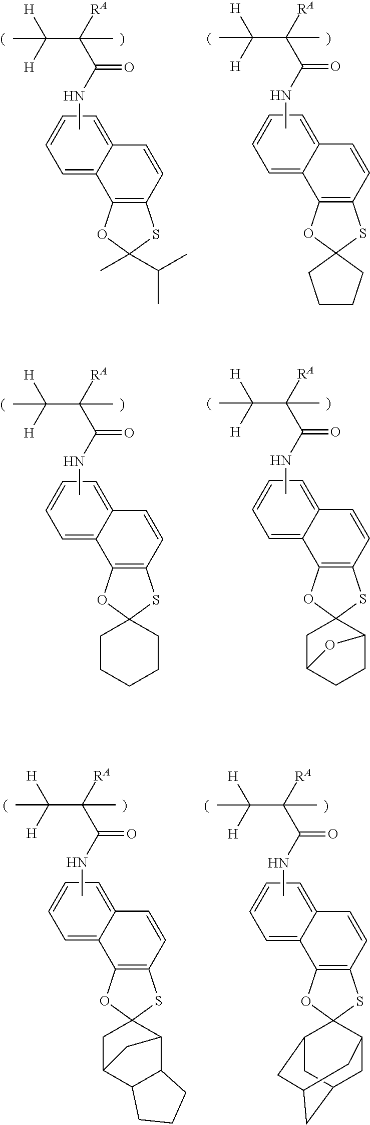

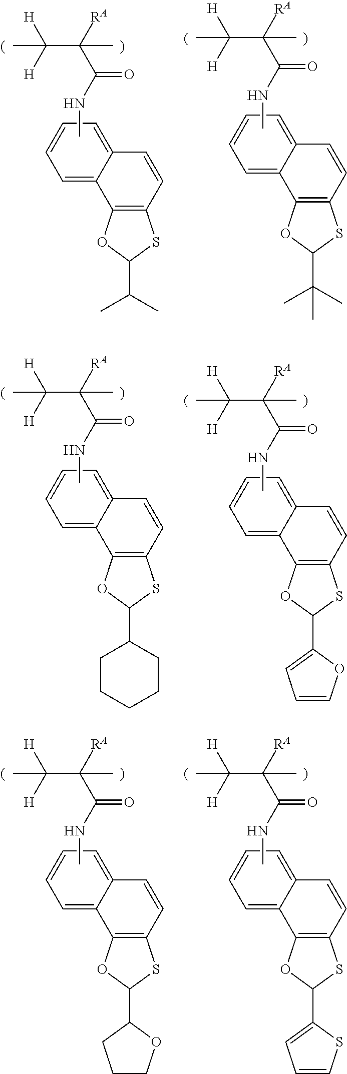

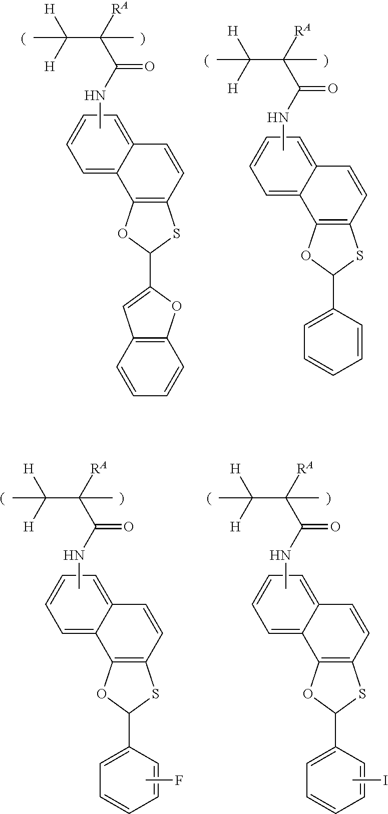

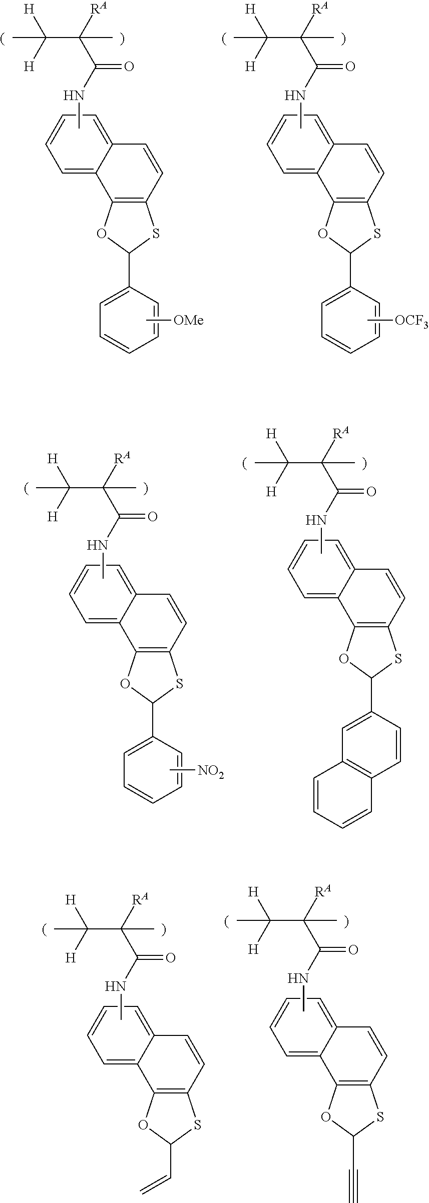

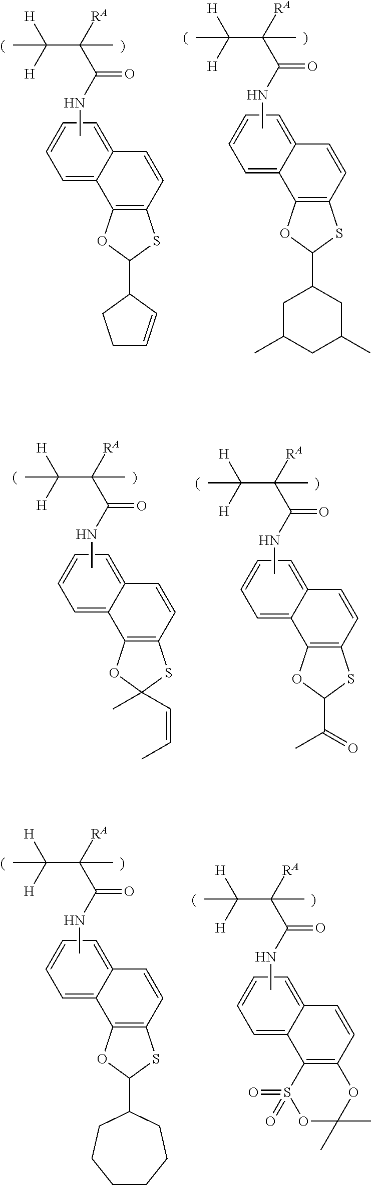

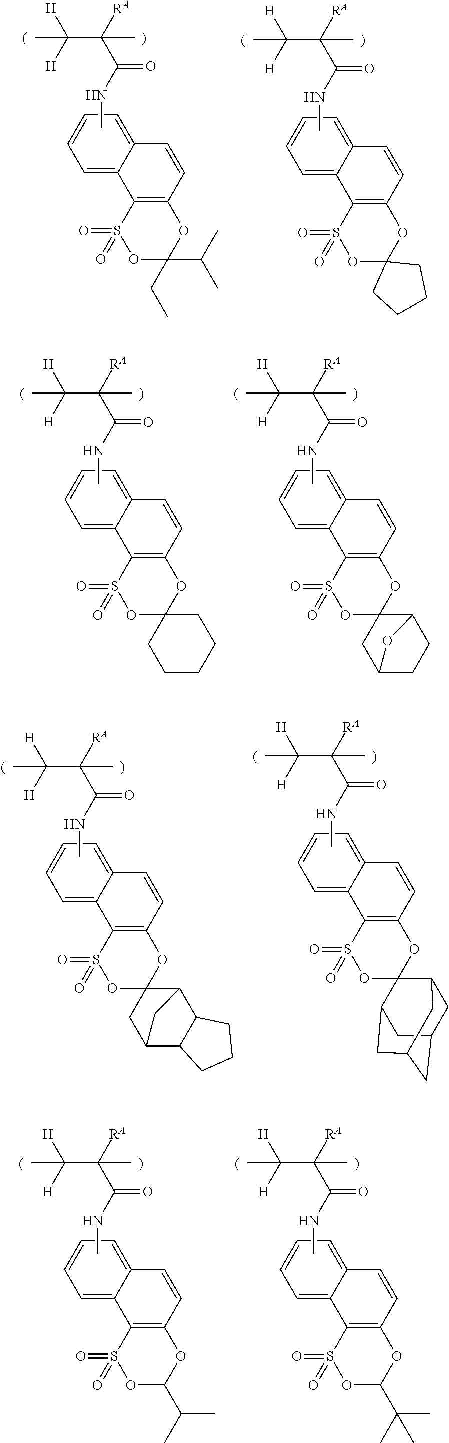

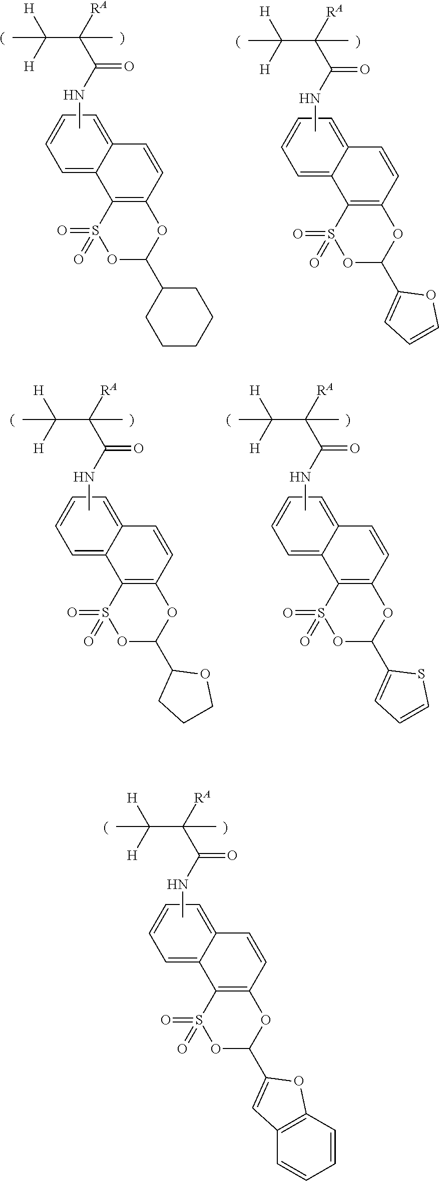

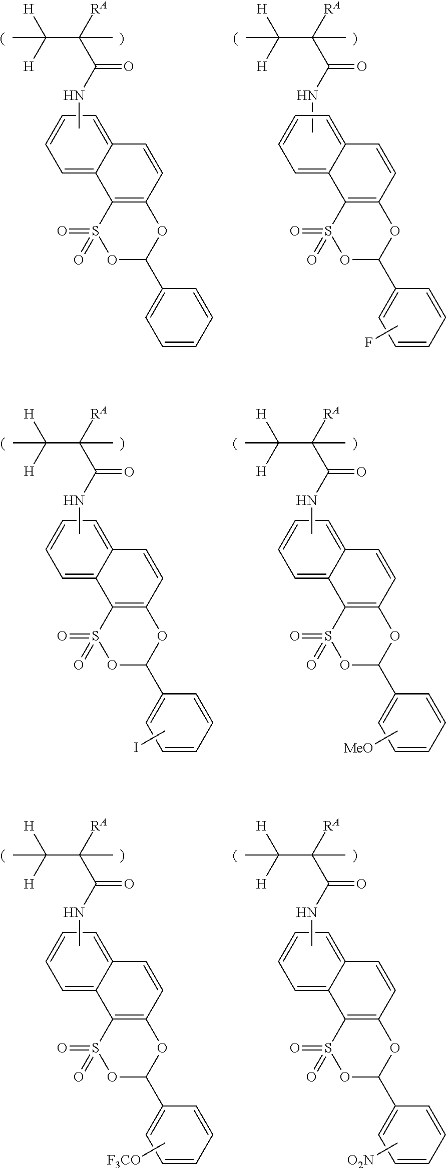

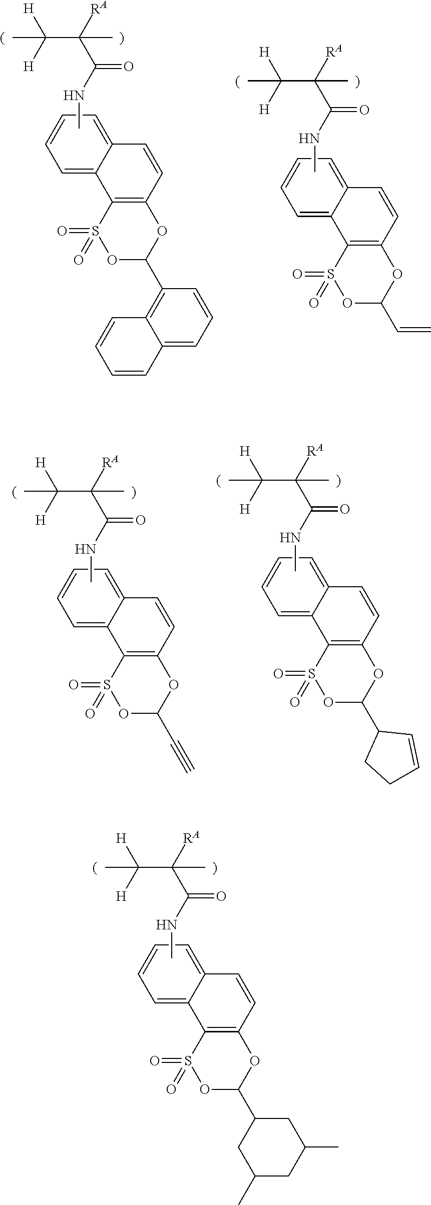

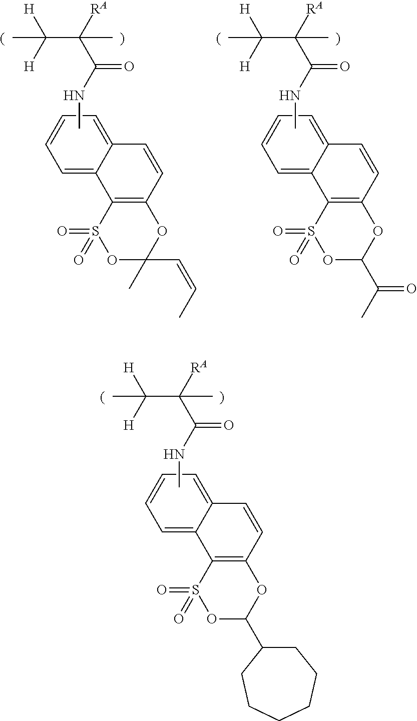

The base polymer may contain a repeat unit having the following formula (b1) (hereinafter, also referred to as a repeat unit b1) or a repeat unit having the following formula (b2) (hereinafter, also referred to as a repeat unit b2).

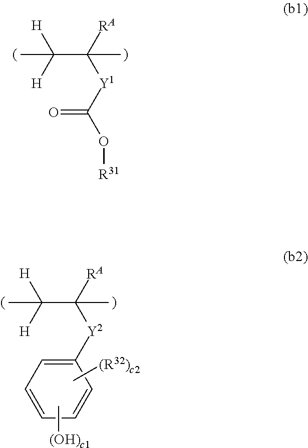

In the formulas (b1) and (b2), R^{A}s are each independently a hydrogen atom, a fluorine atom, a methyl group, or a trifluoromethyl group. Y¹ is a single bond, a phenylene group, a naphthylene group, *—C(═O)—O—Y¹¹—, or *—C(═O)—N(H)—Y¹¹—, and the phenylene group or the naphthylene group may have a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom as a substituent. Y¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring. Y² is a single bond or *—C(═O)—O—. * represents a bond with a carbon atom of a main chain. R³¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—). R³² is a halogen atom, a carboxy group, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. When c2 is 2, 3, or 4, R³²s may be the same as or different from each other. c1 is 1, 2, 3, or 4. c2 is 0, 1, 2, 3, or 4. Provided that 1≤c1+c2≤5.

Specific examples of the repeat unit b1 include, but are not limited to, those listed below. Note that, in the following formulas, R^{A} is as defined above.

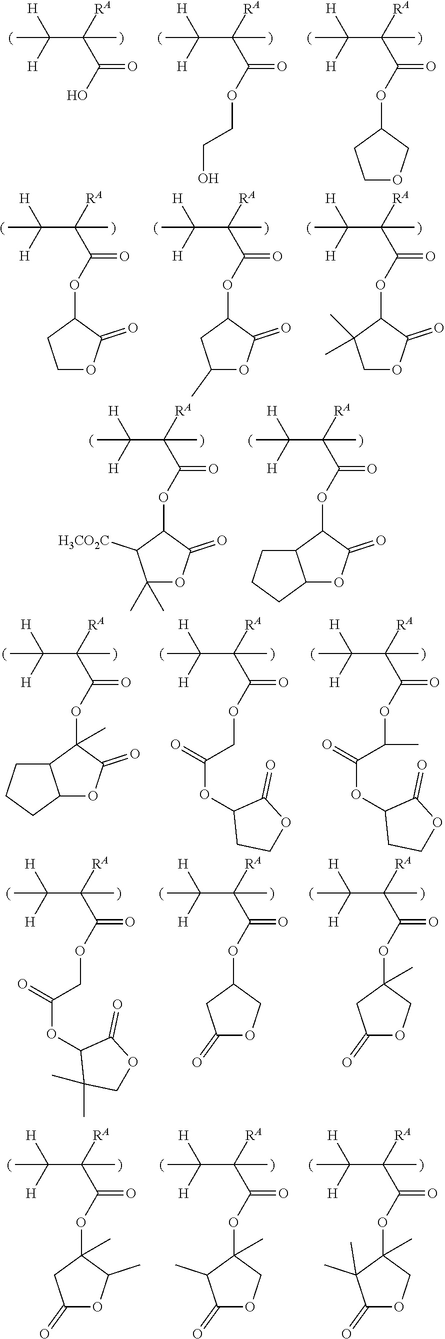

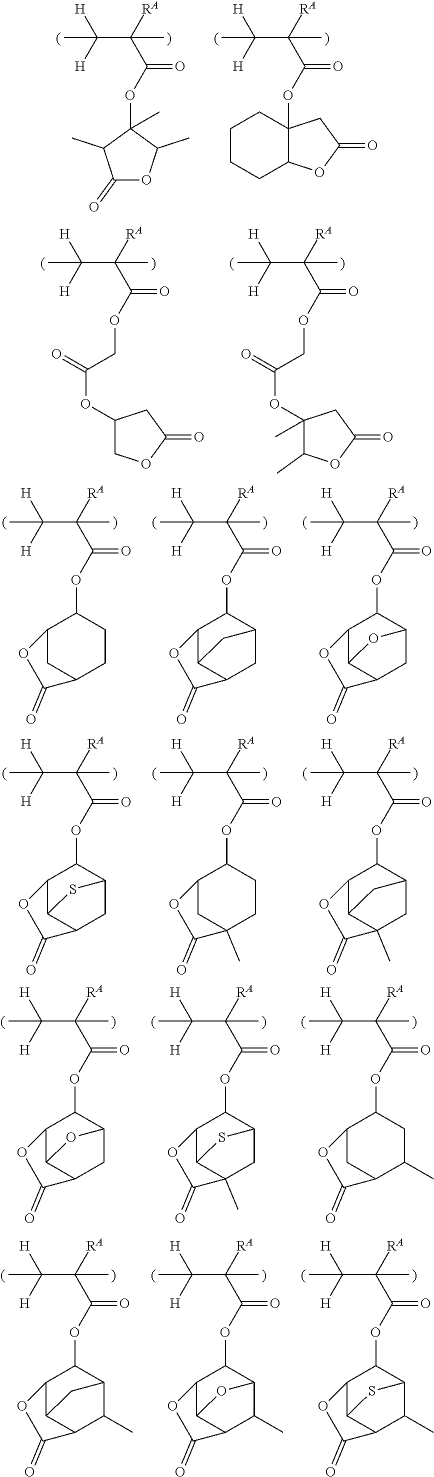

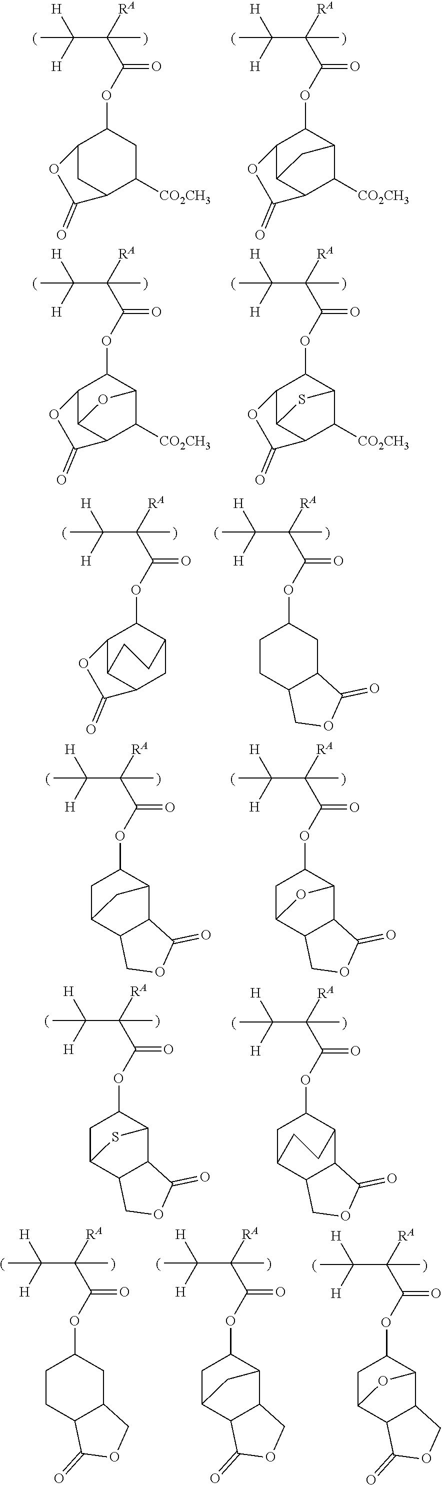

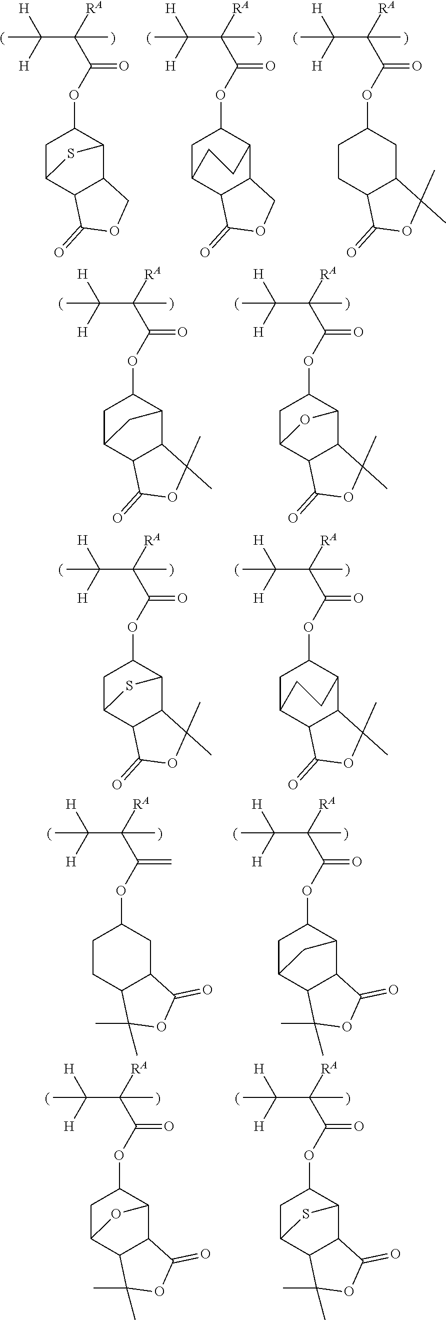

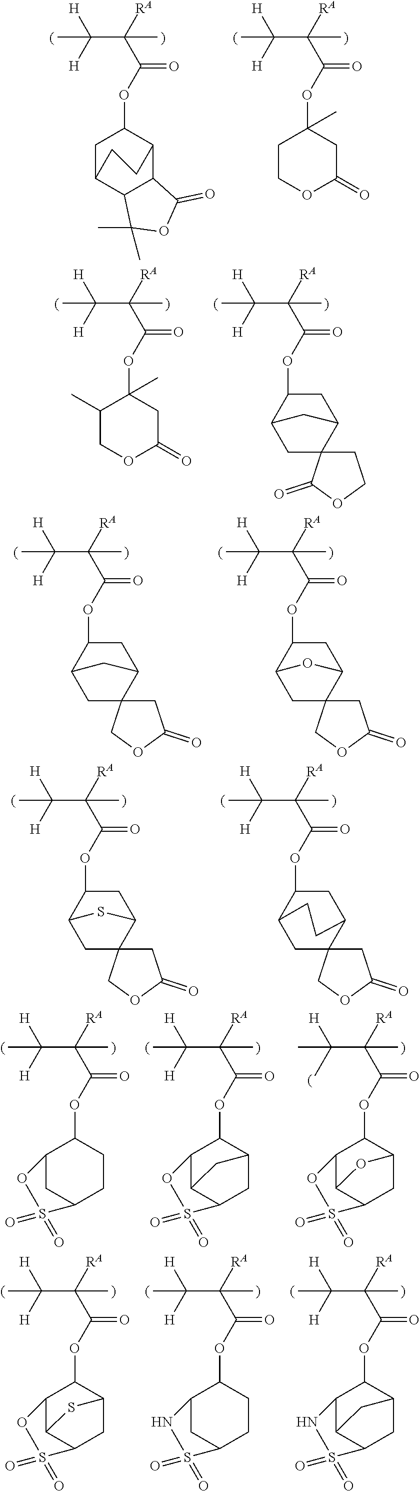

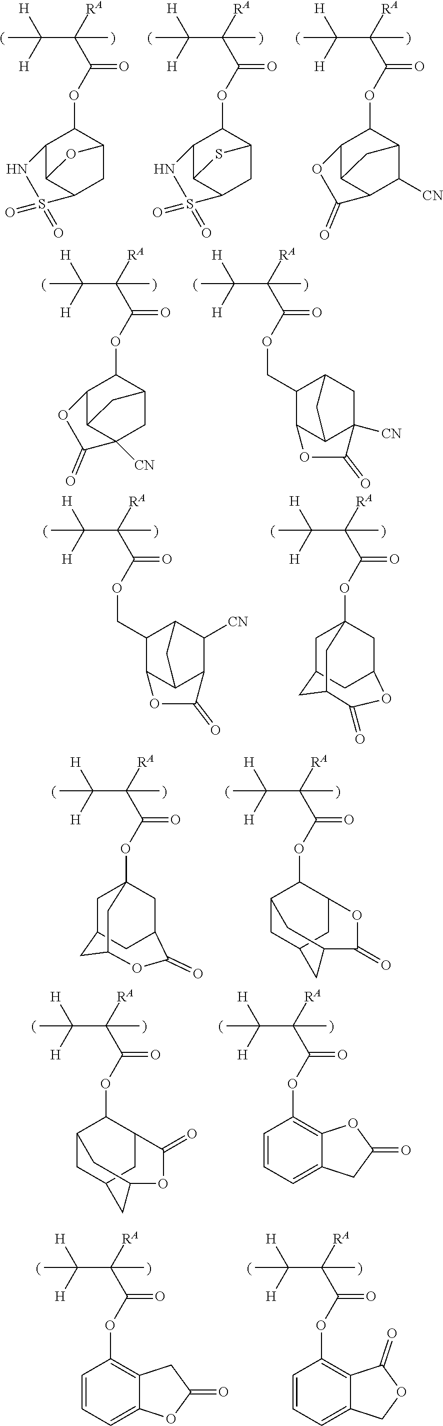

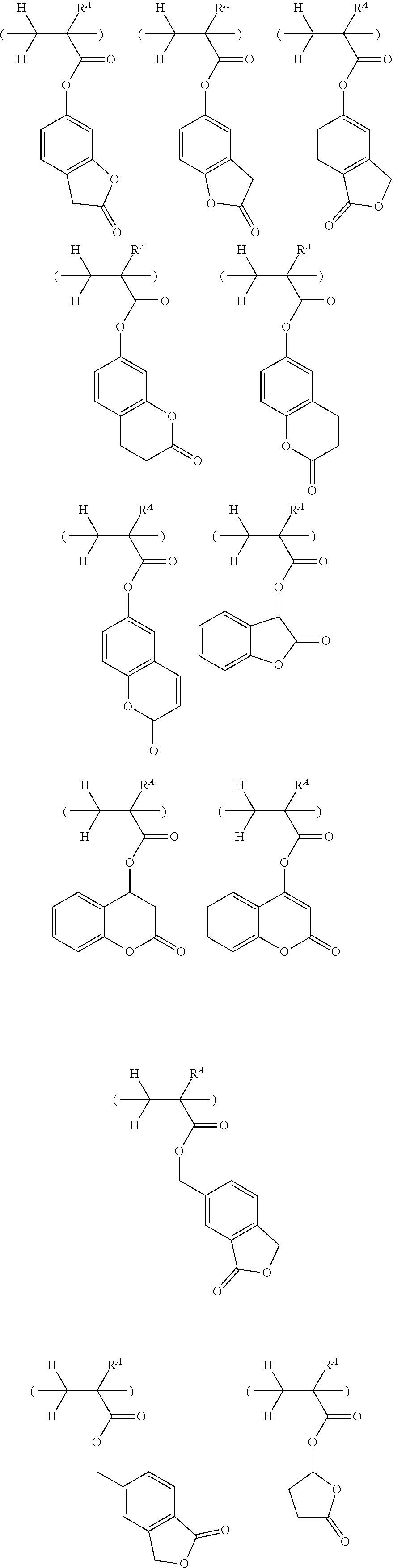

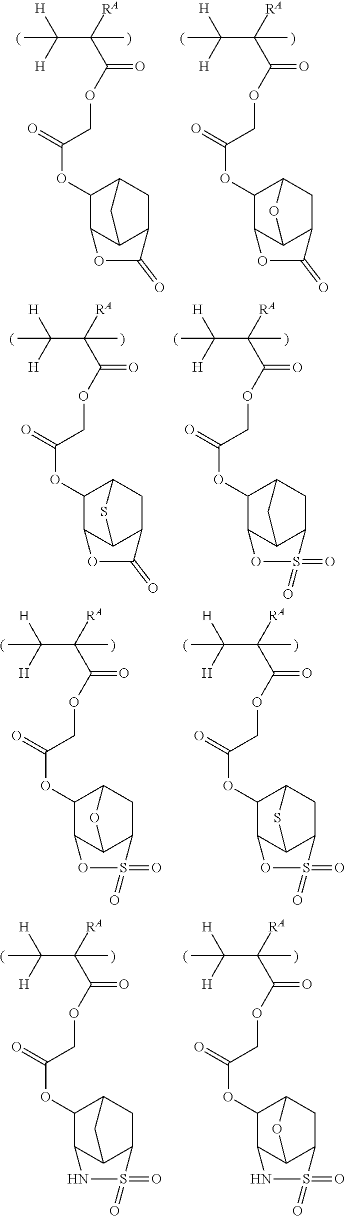

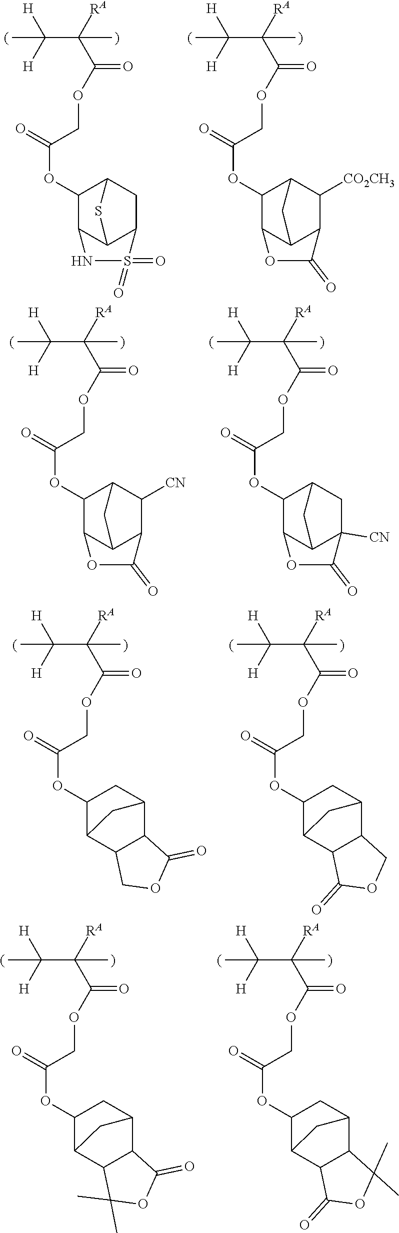

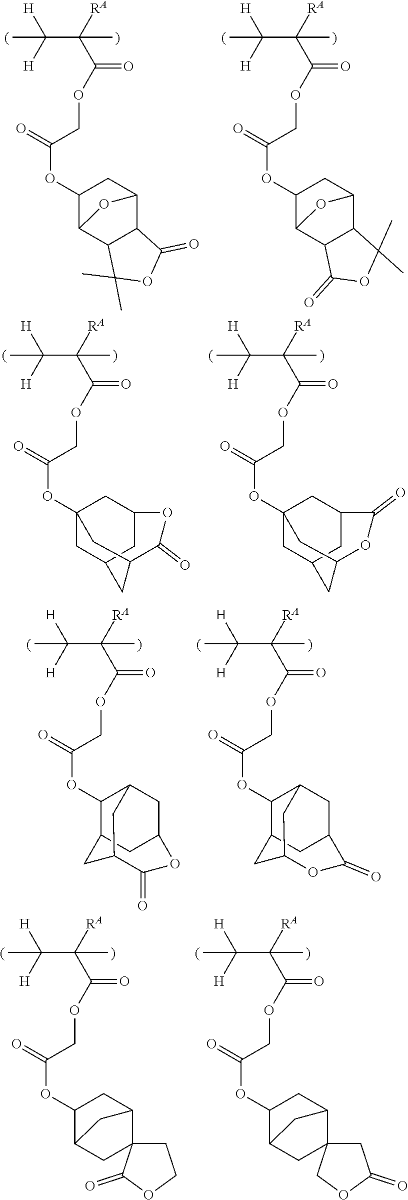

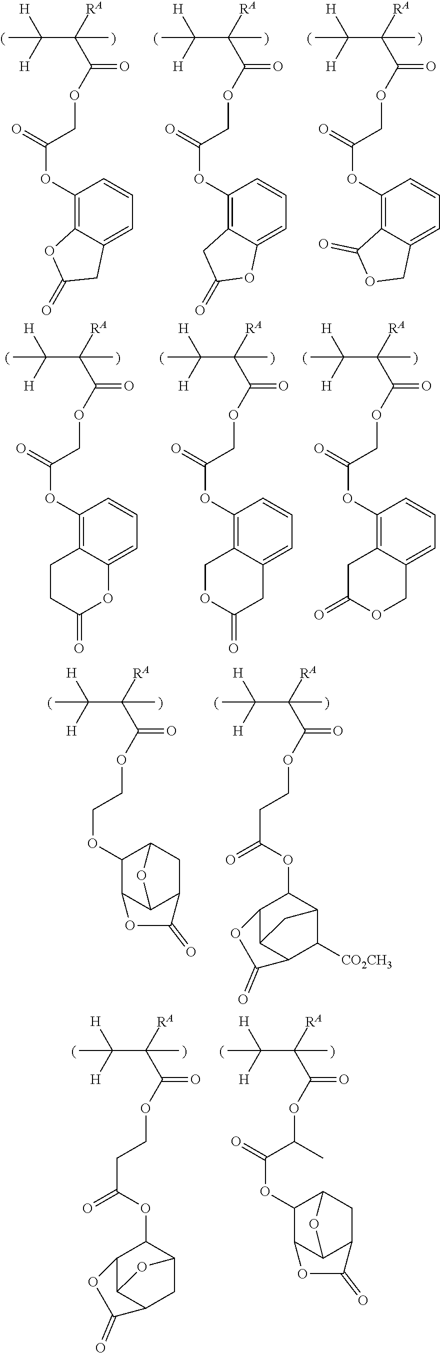

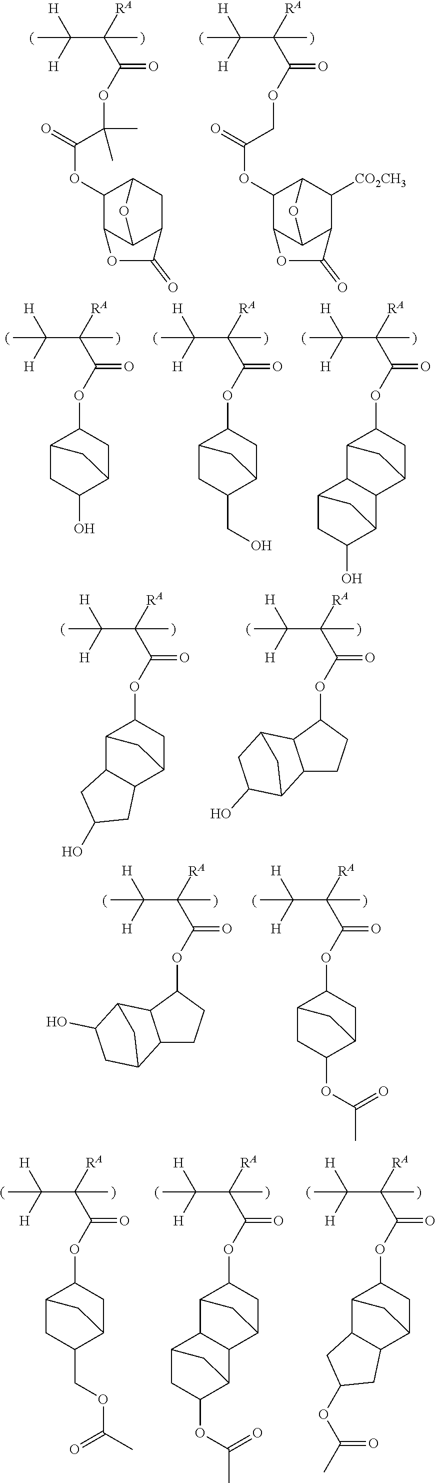

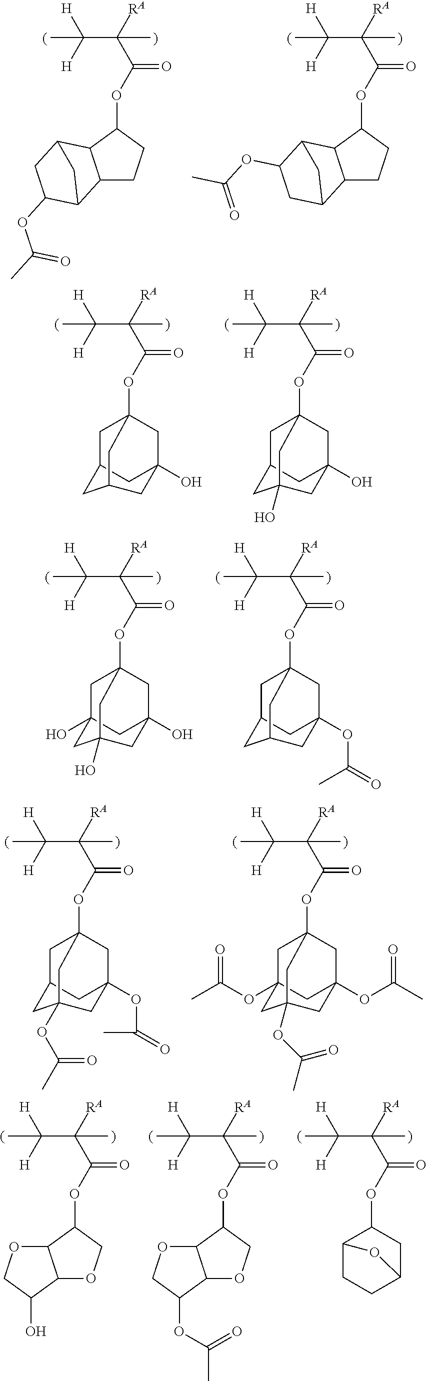

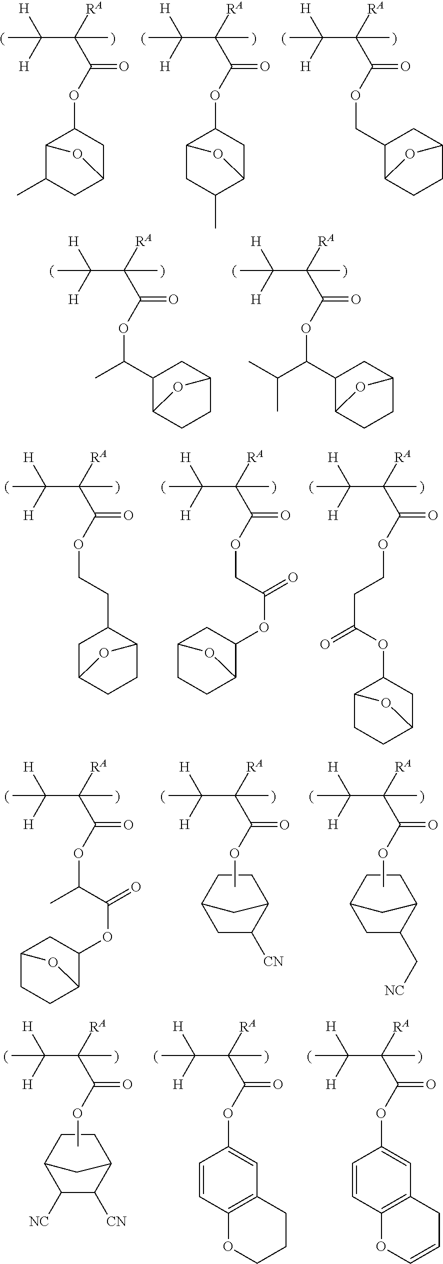

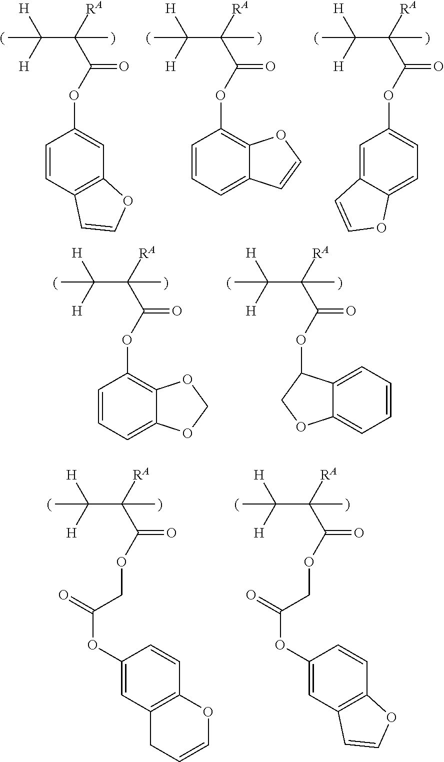

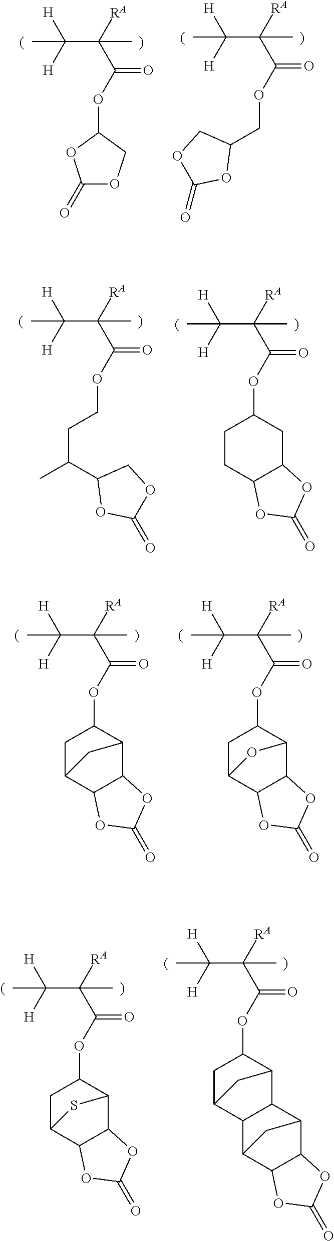

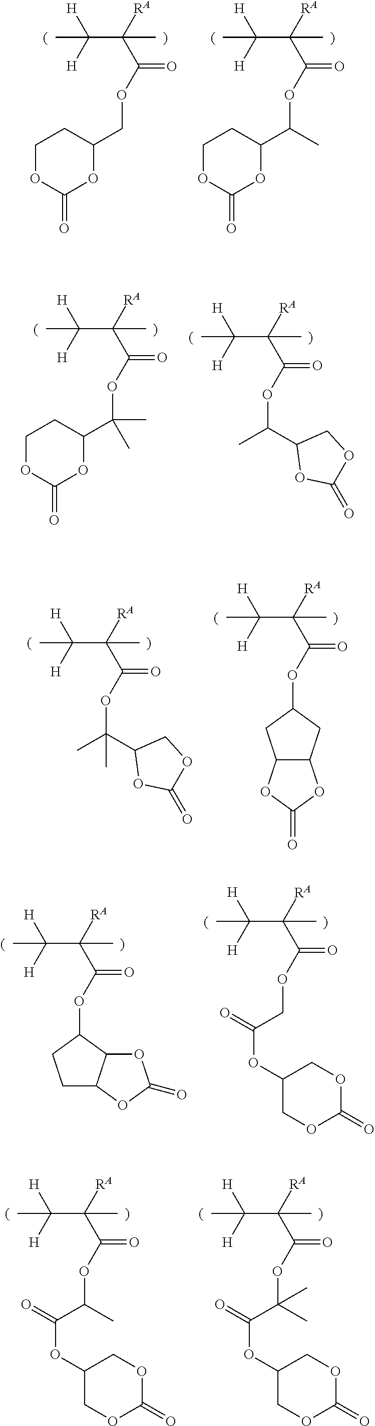

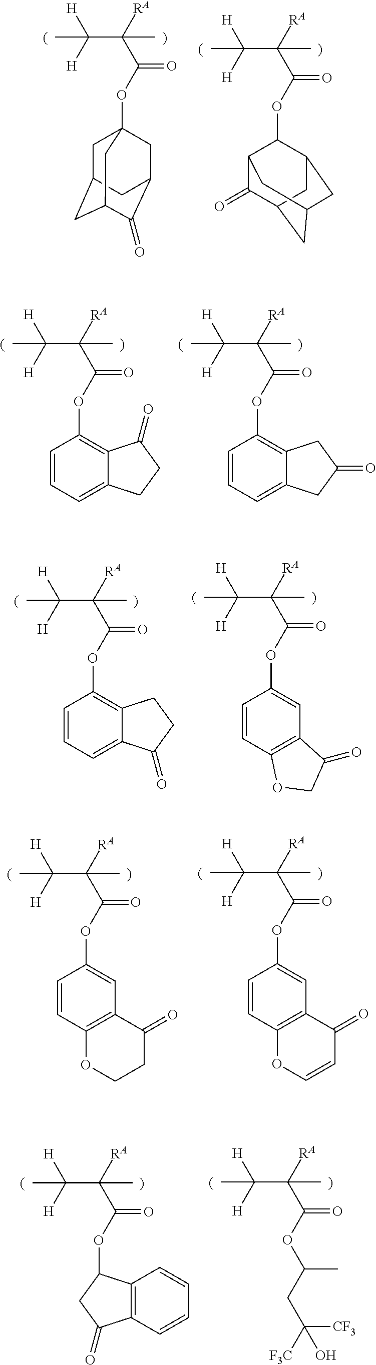

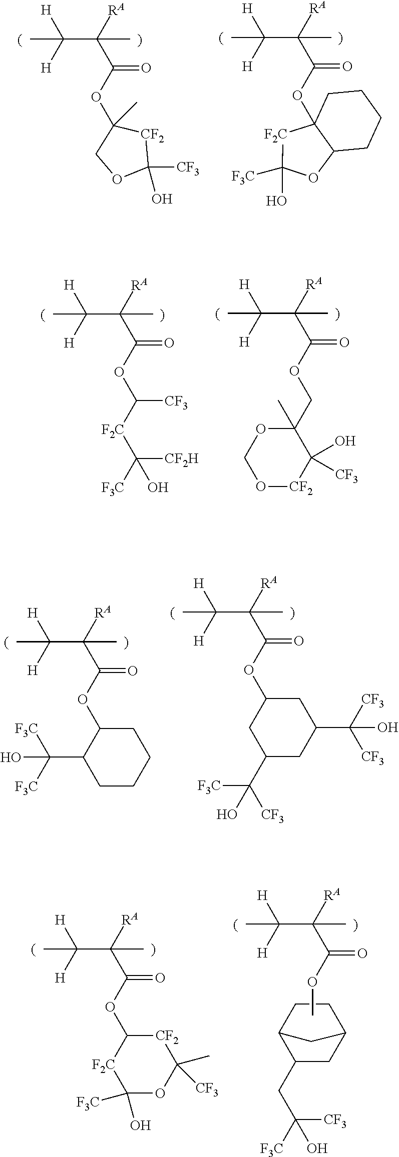

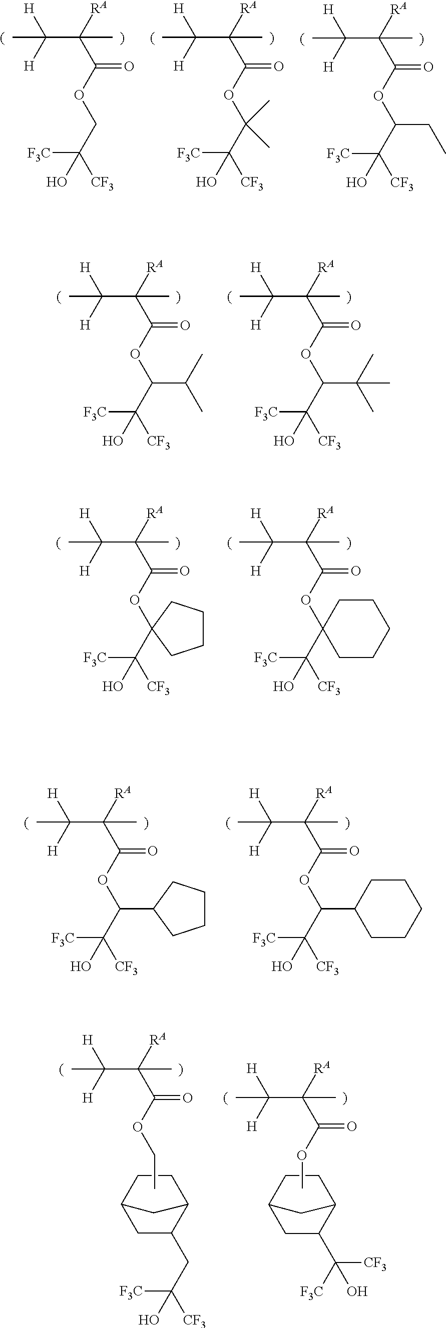

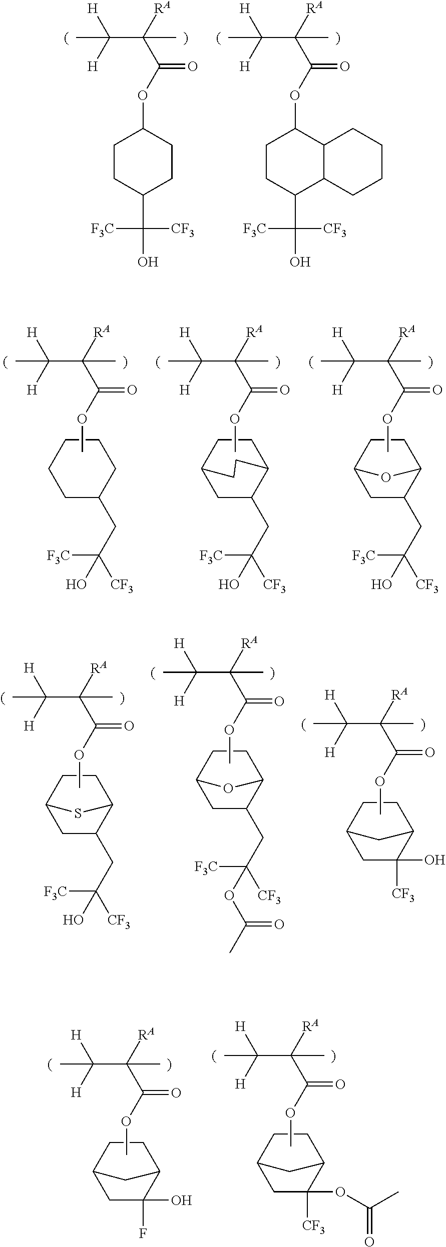

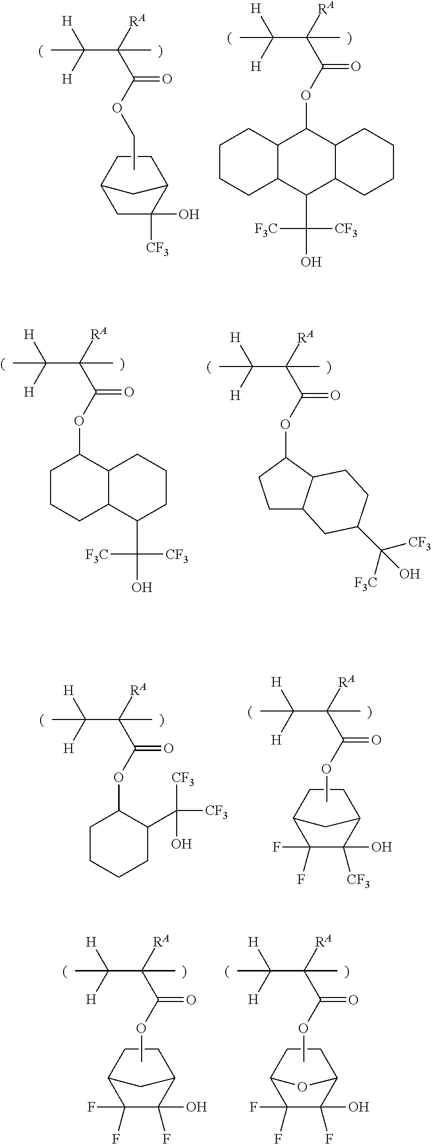

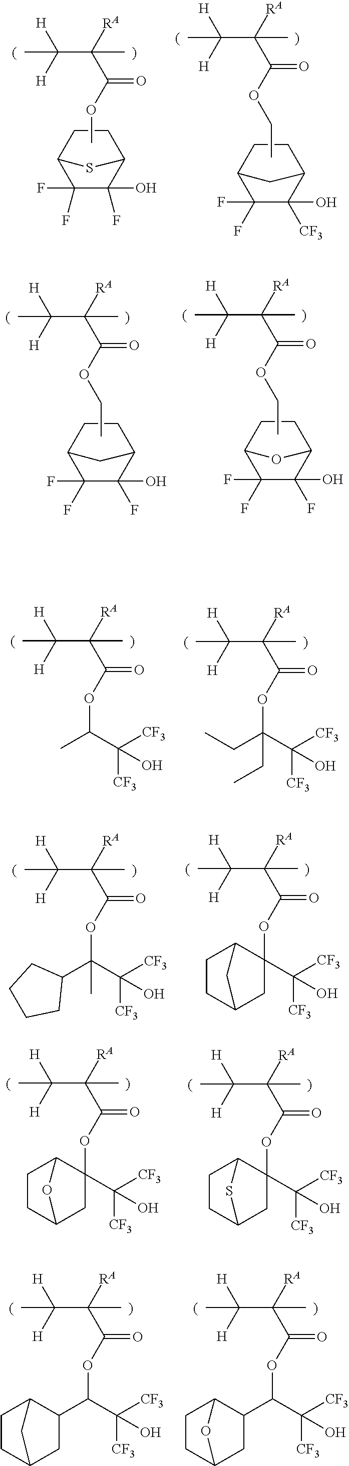

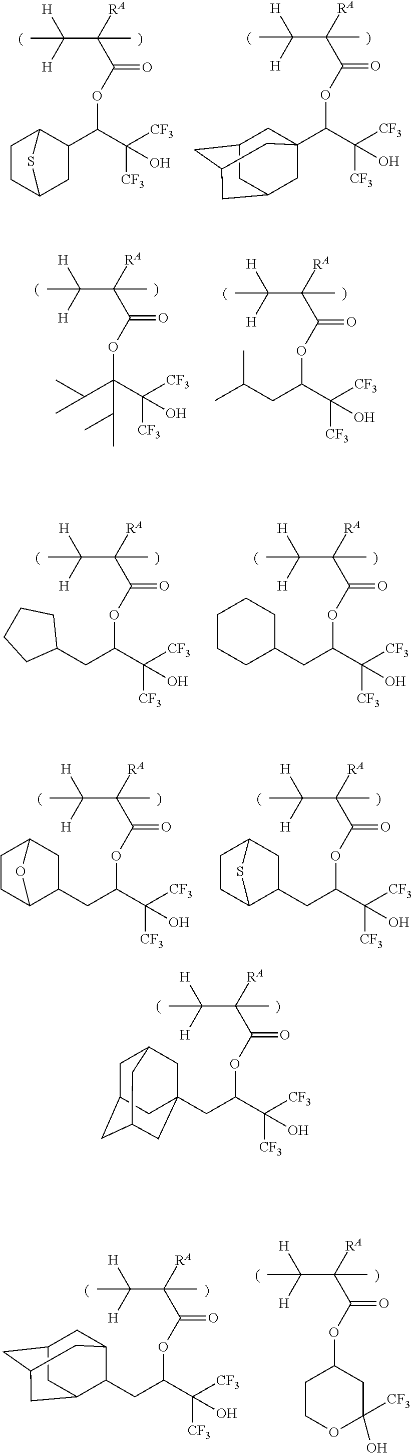

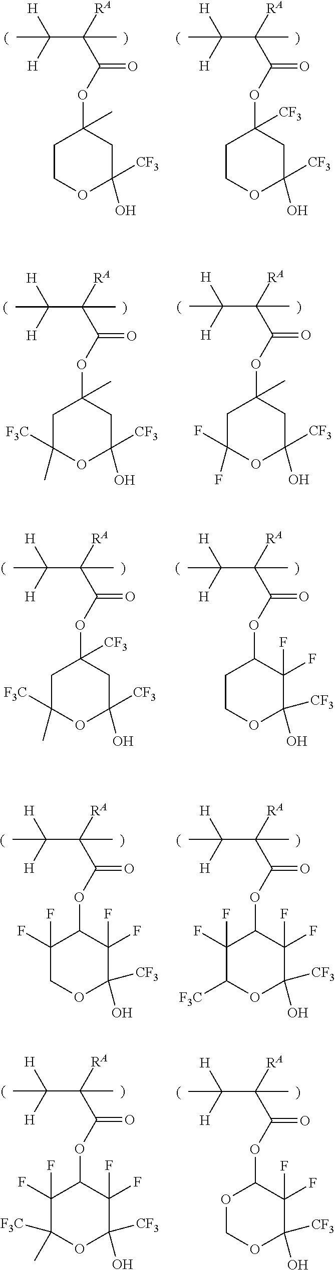

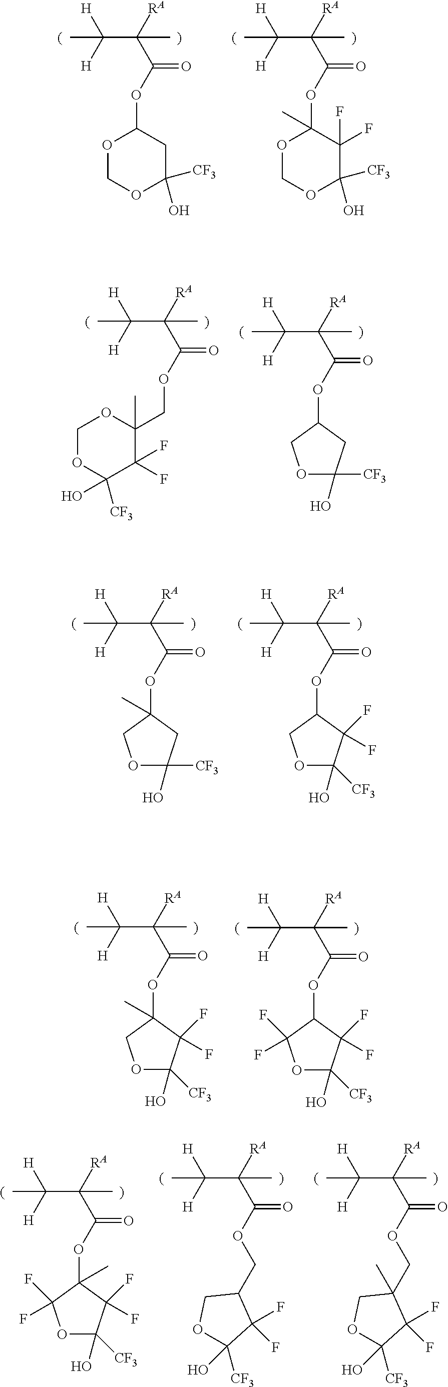

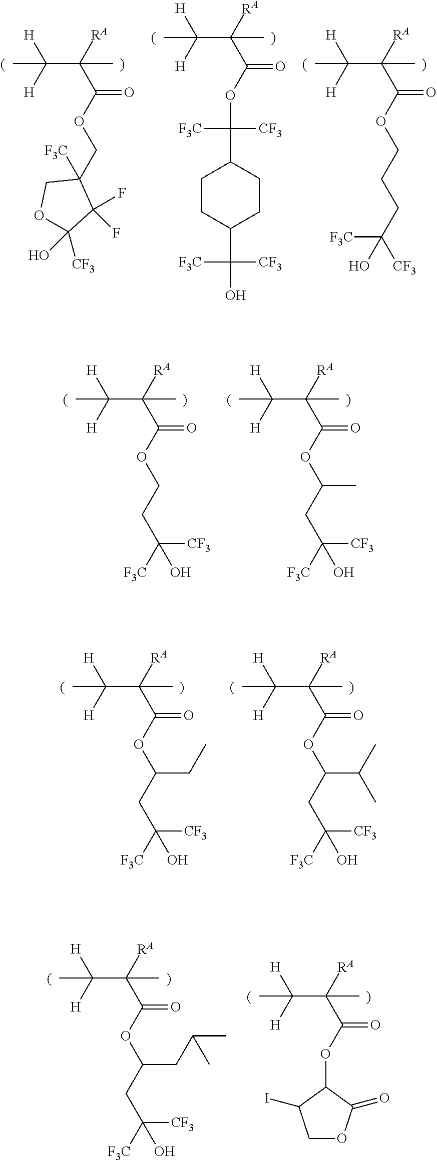

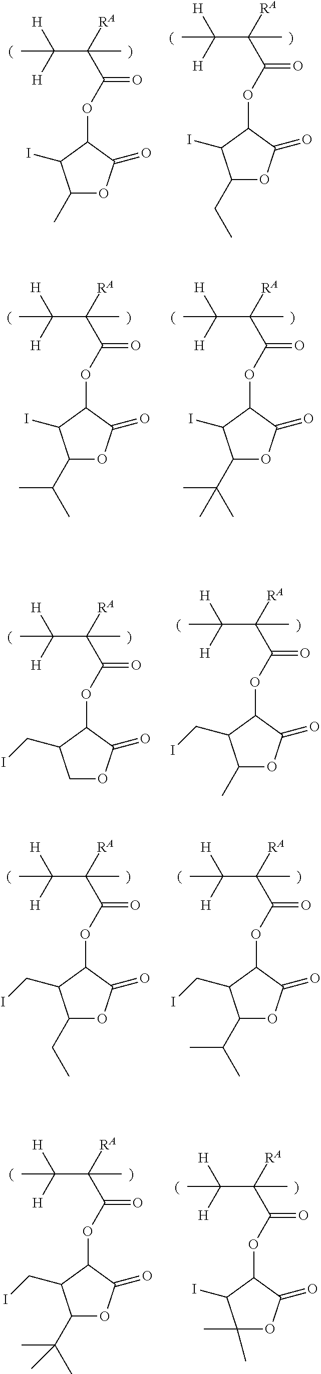

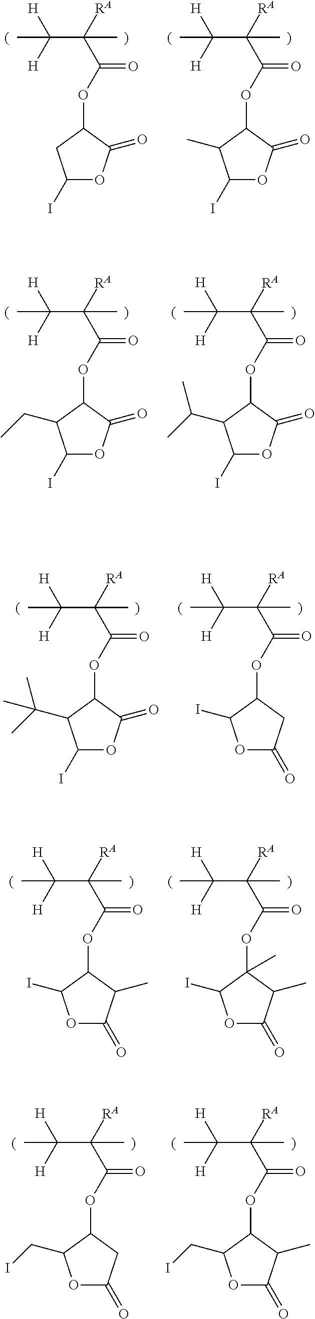

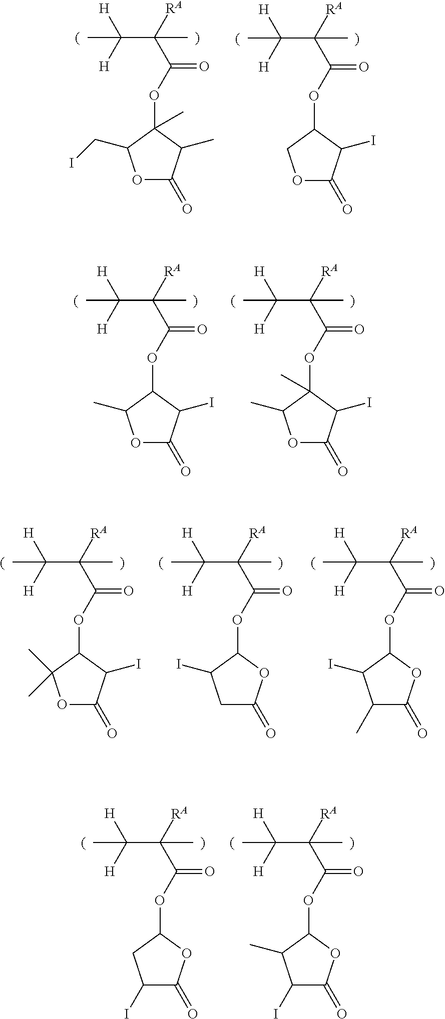

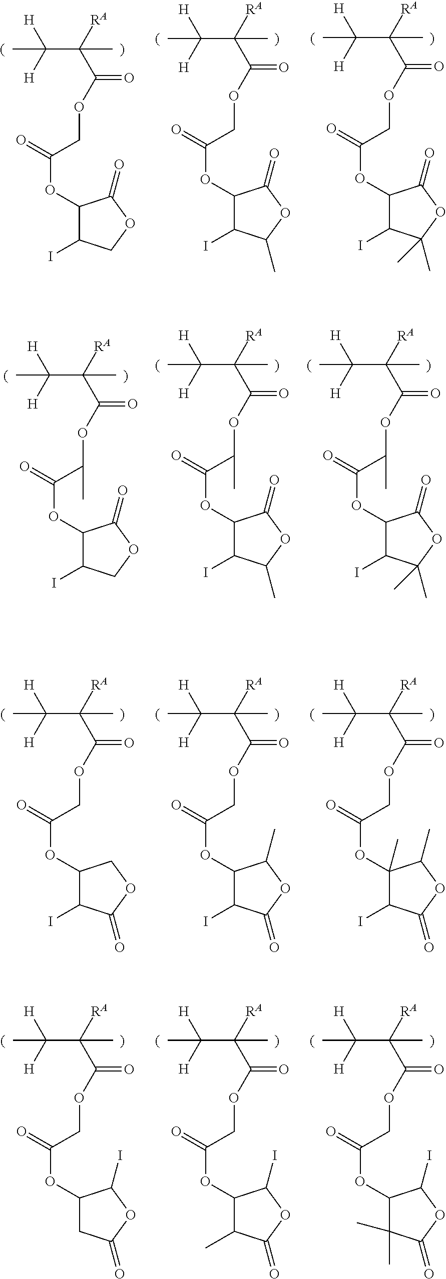

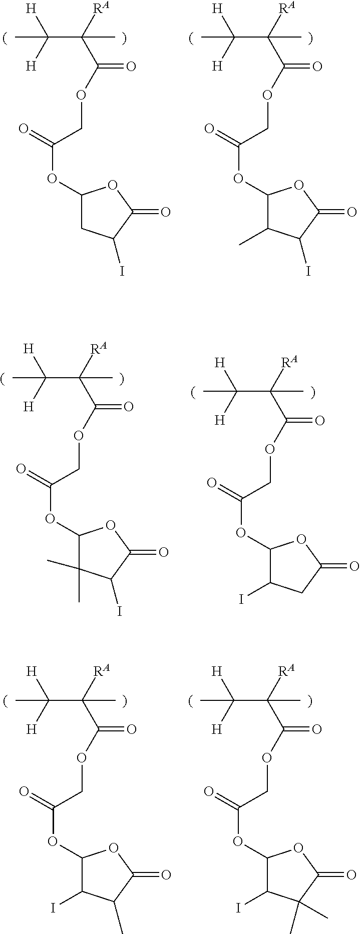

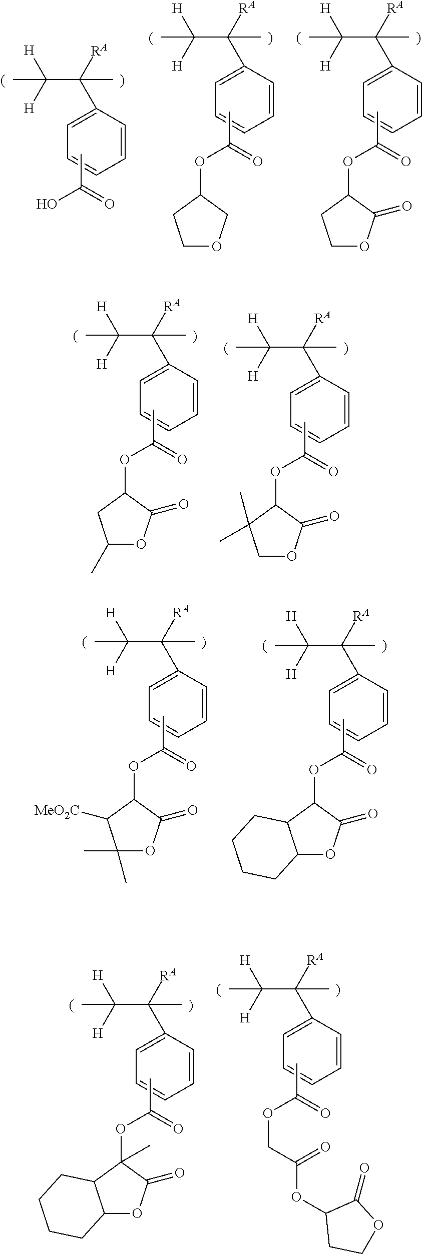

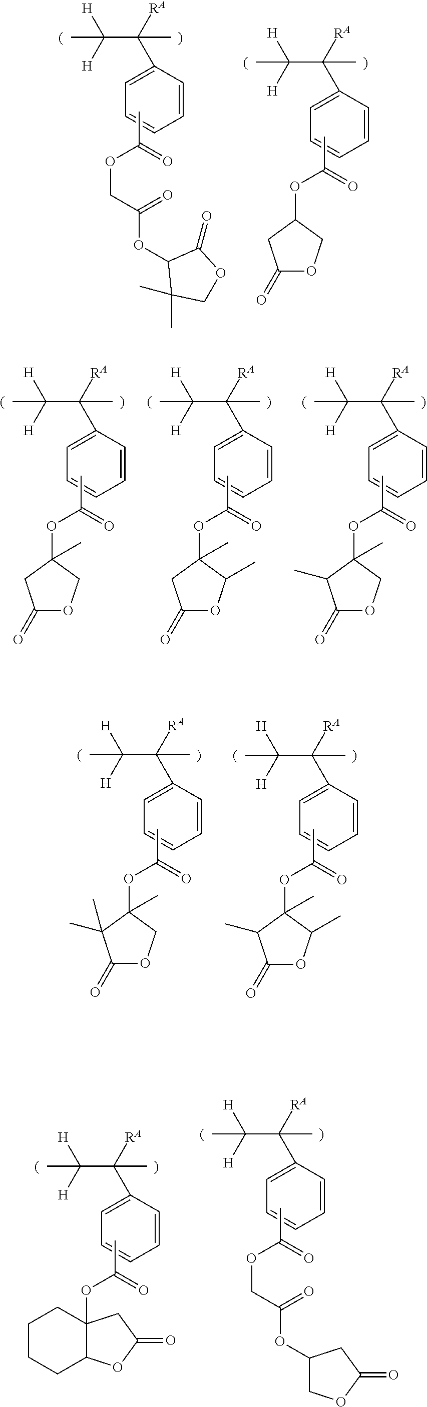

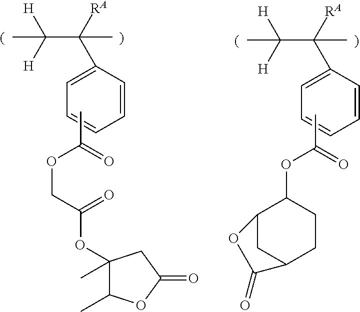

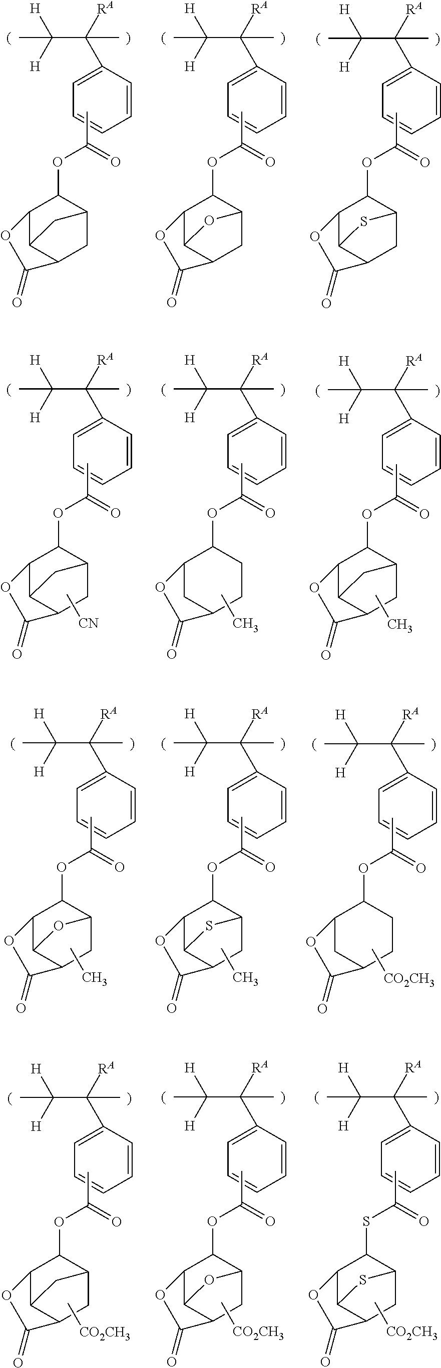

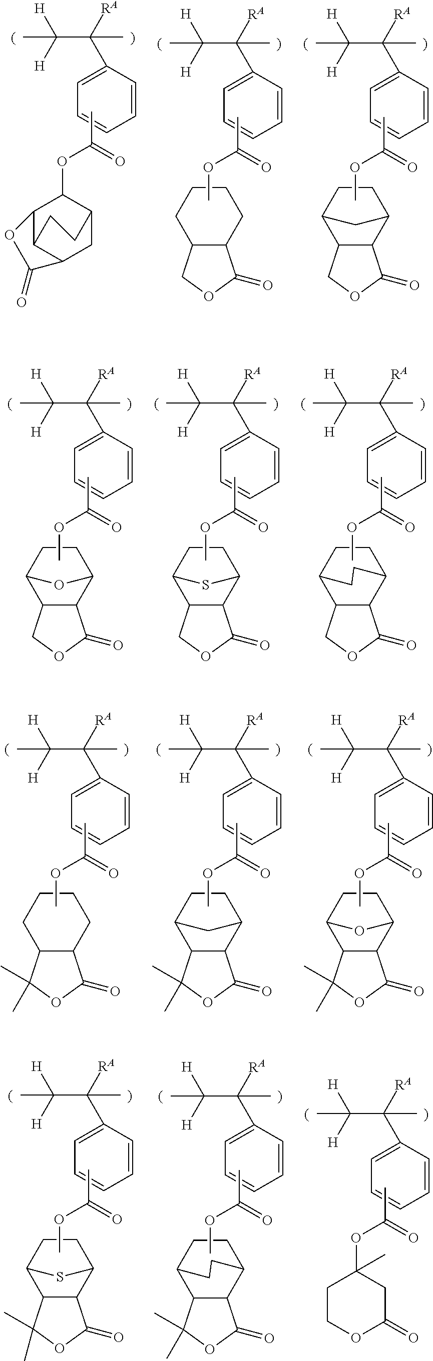

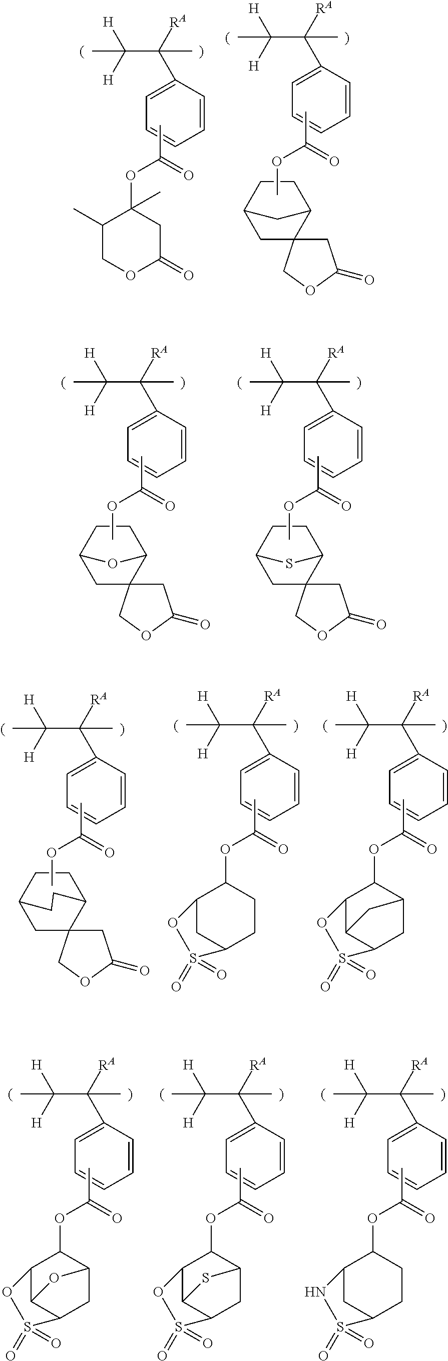

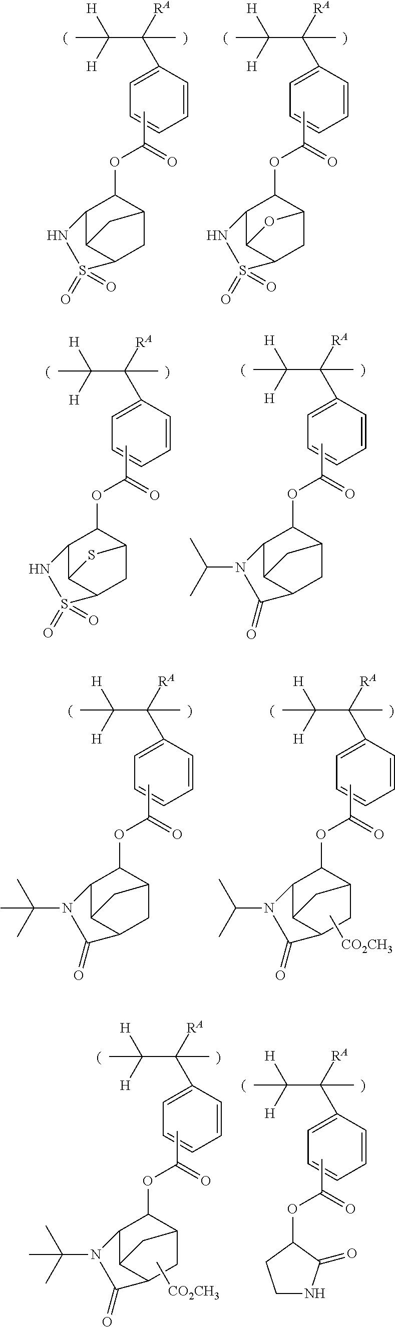

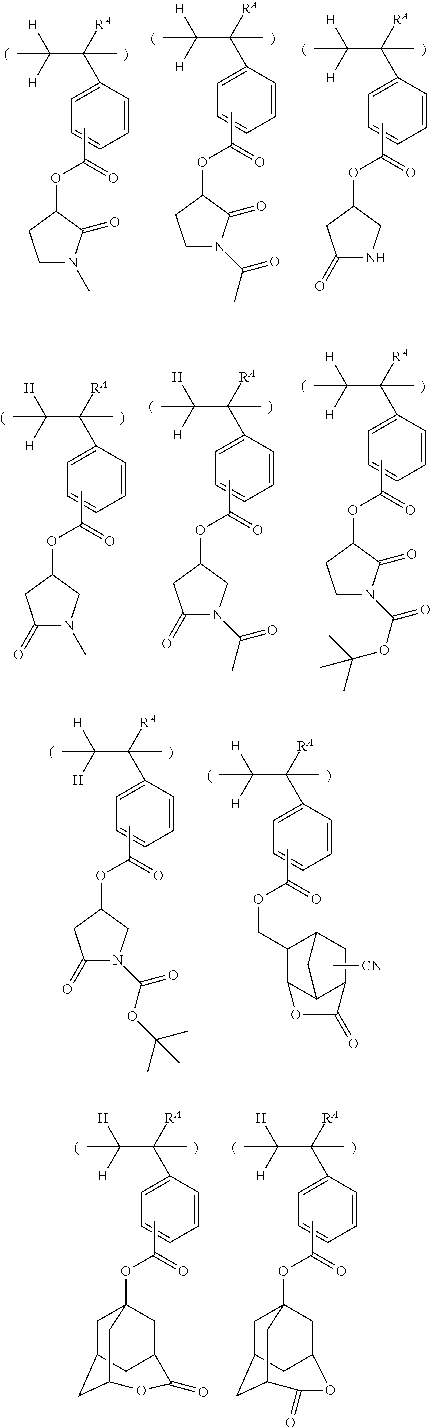

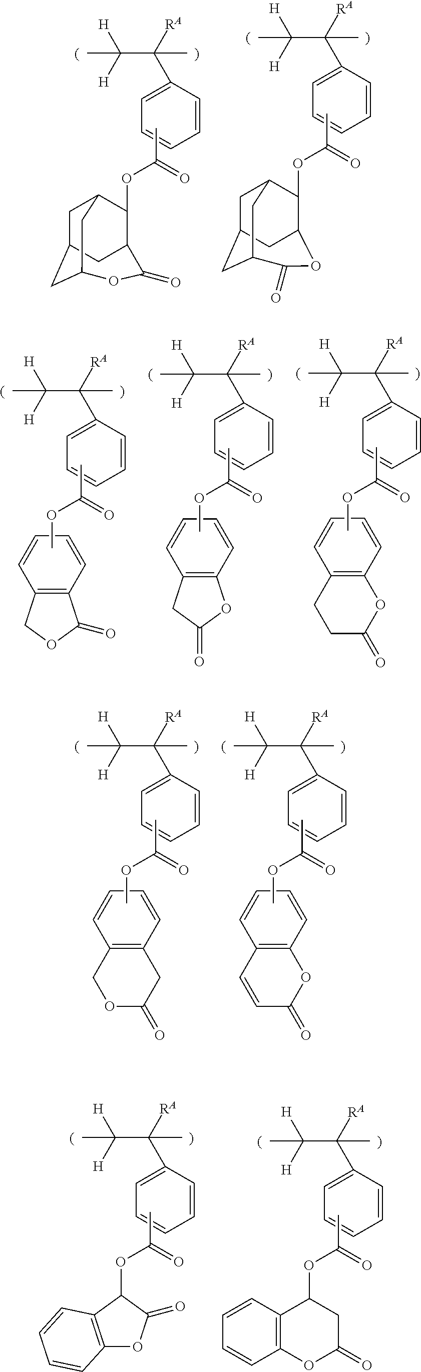

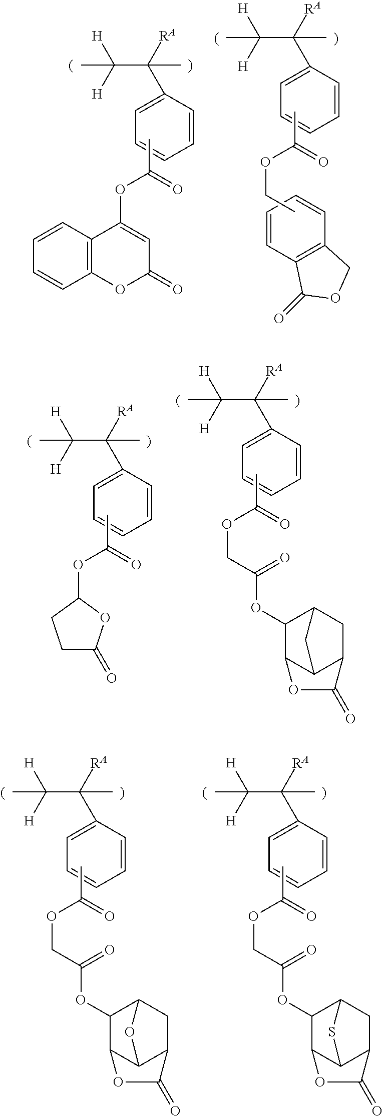

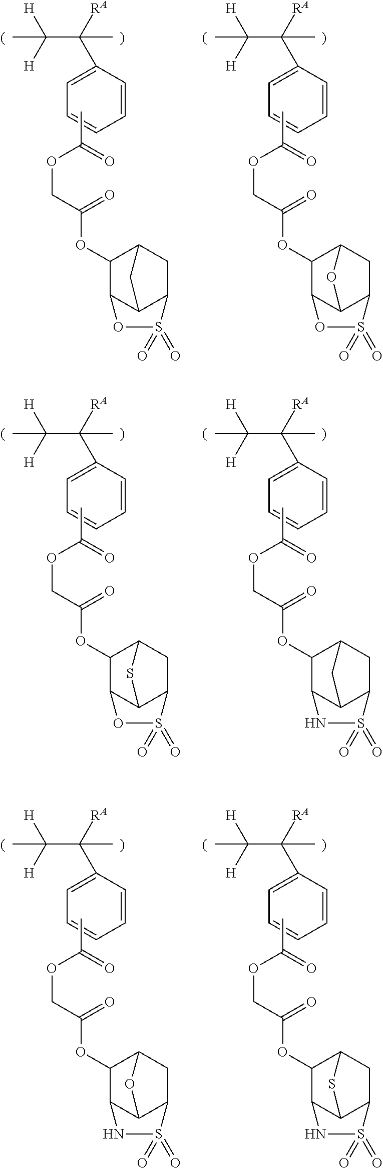

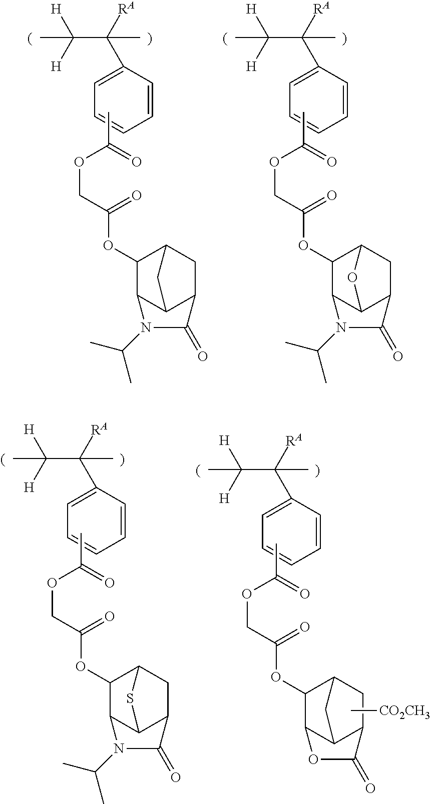

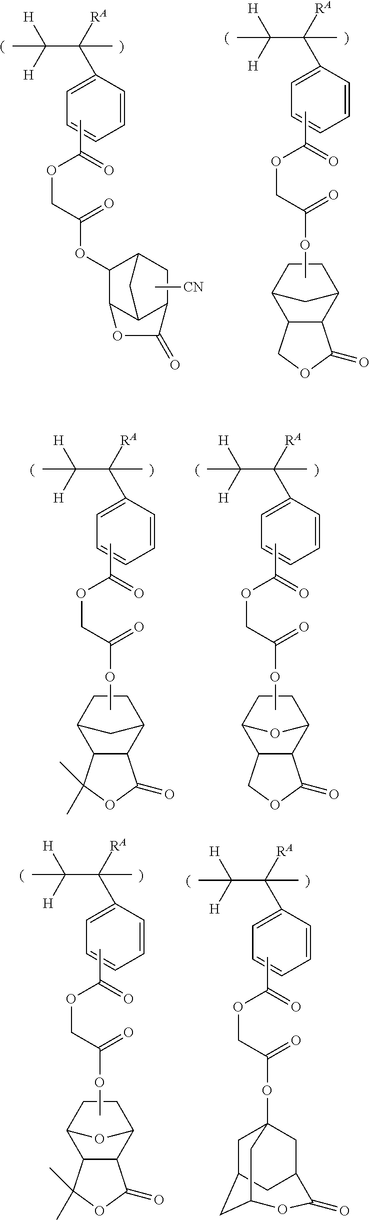

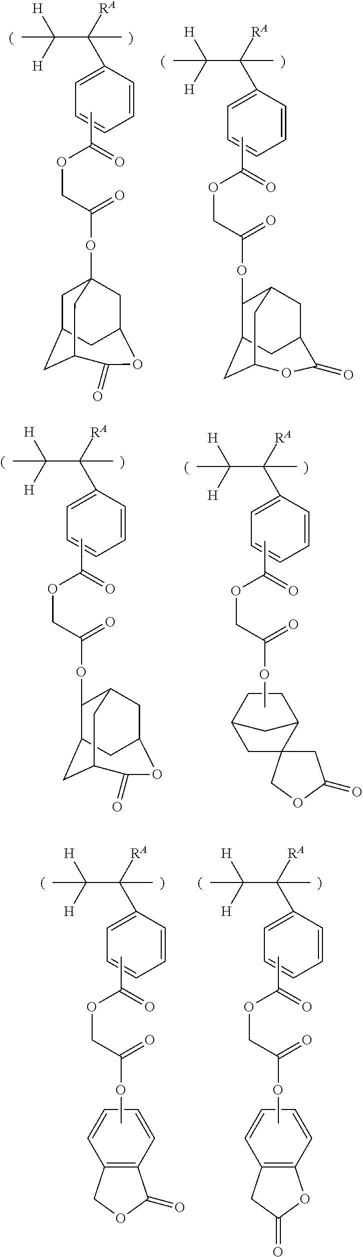

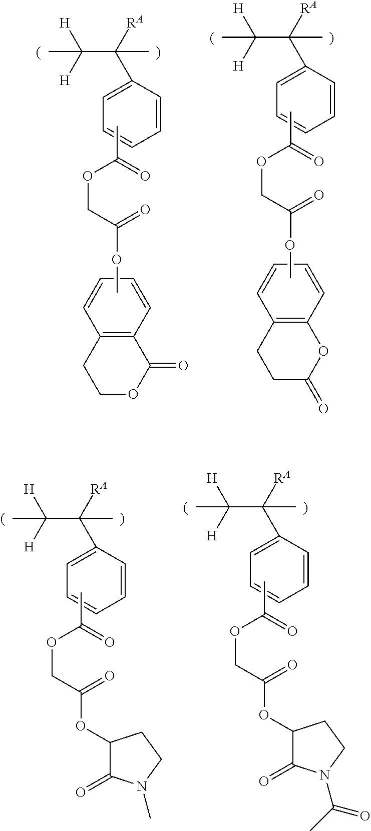

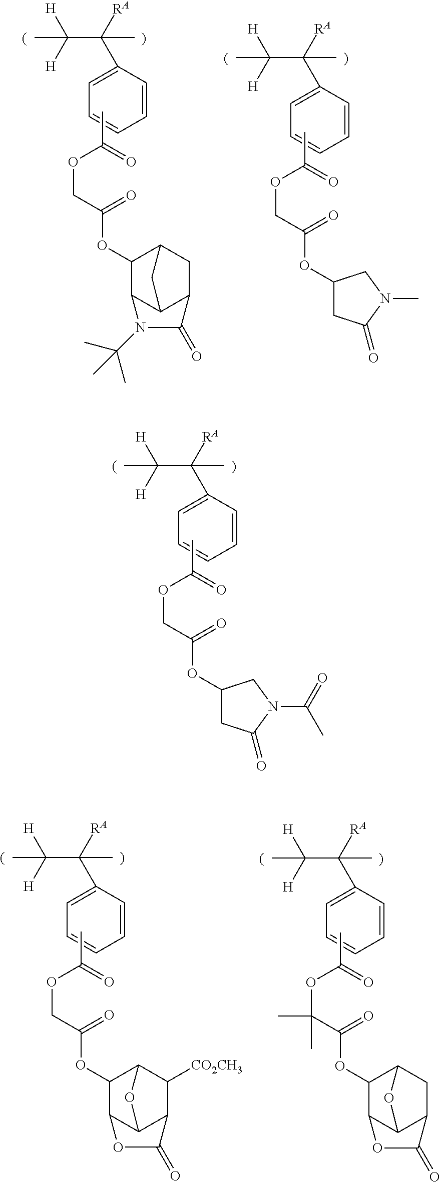

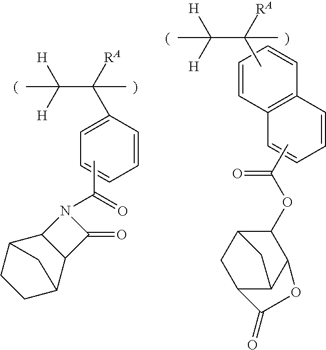

Specific examples of the repeat unit b2 include, but are not limited to, those listed below. Note that, in the following formulas, RA is as defined above.

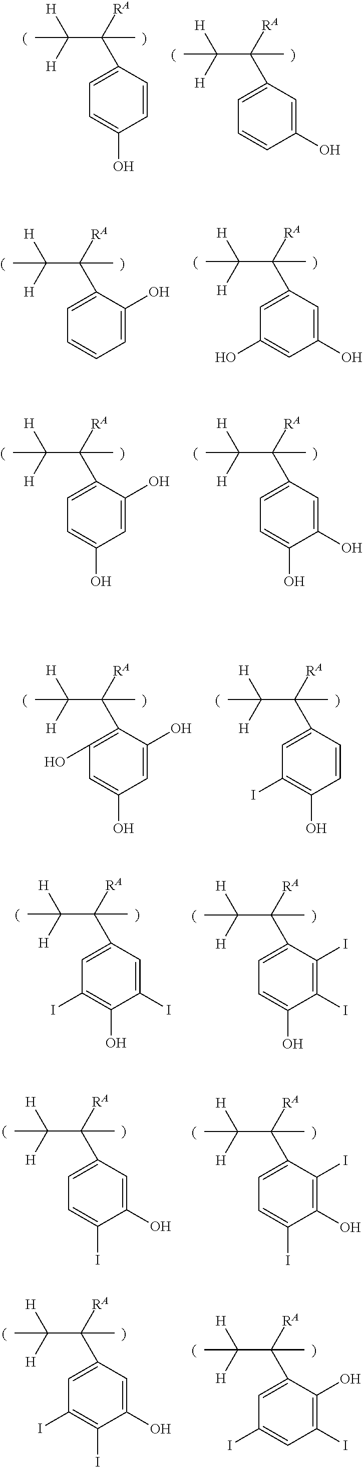

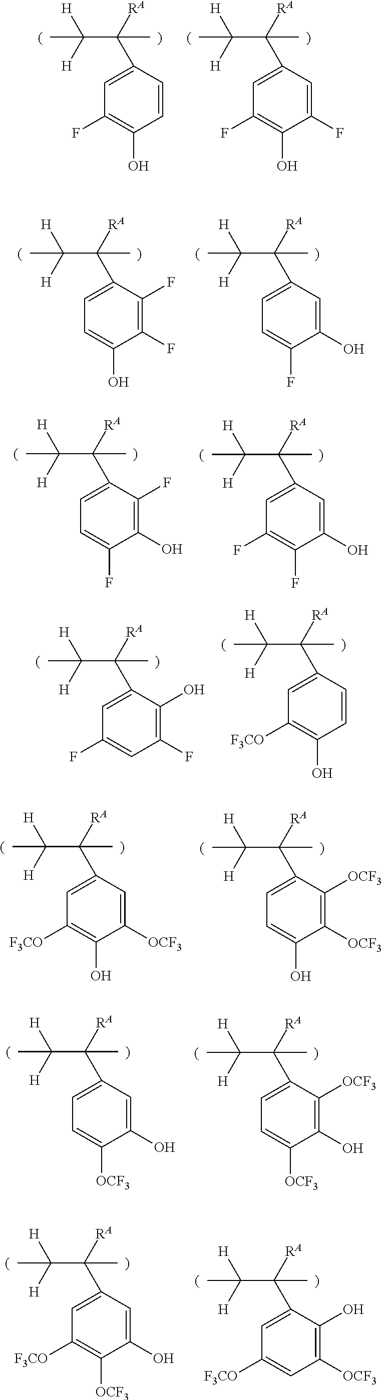

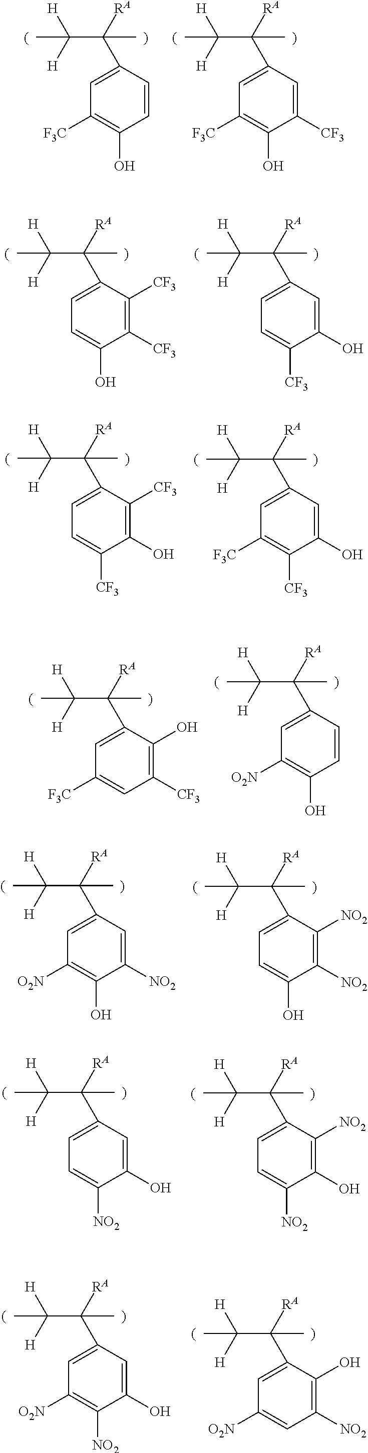

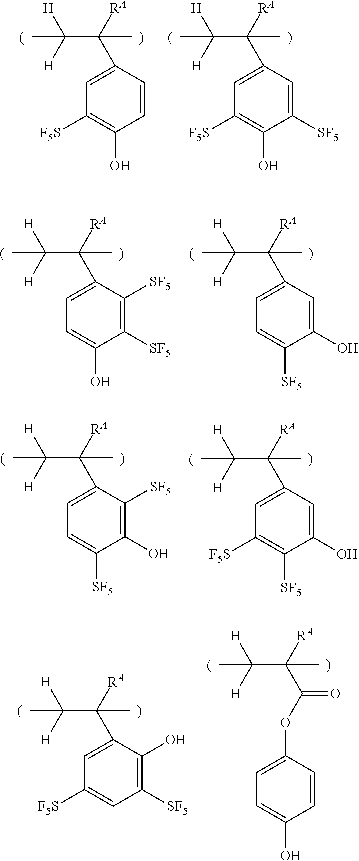

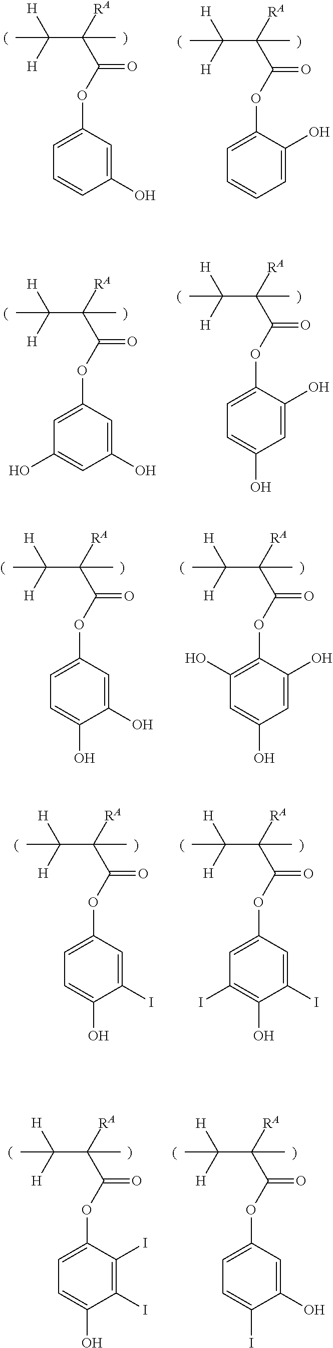

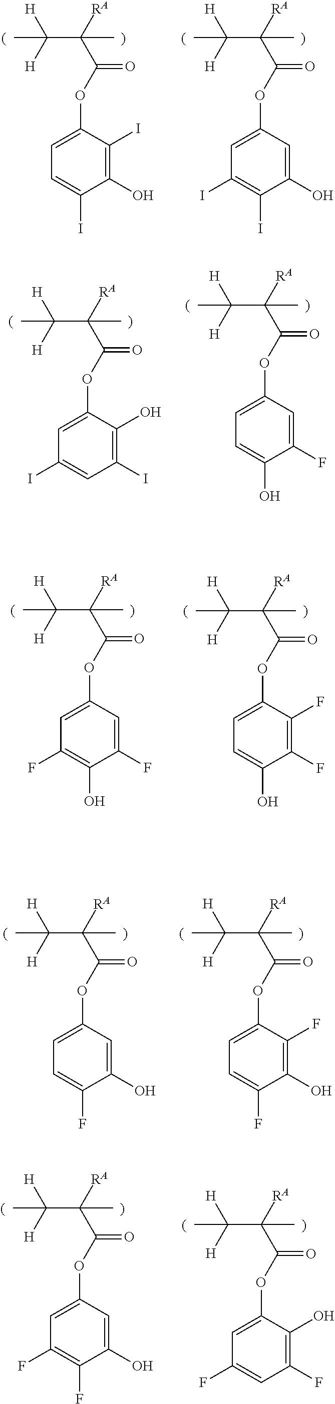

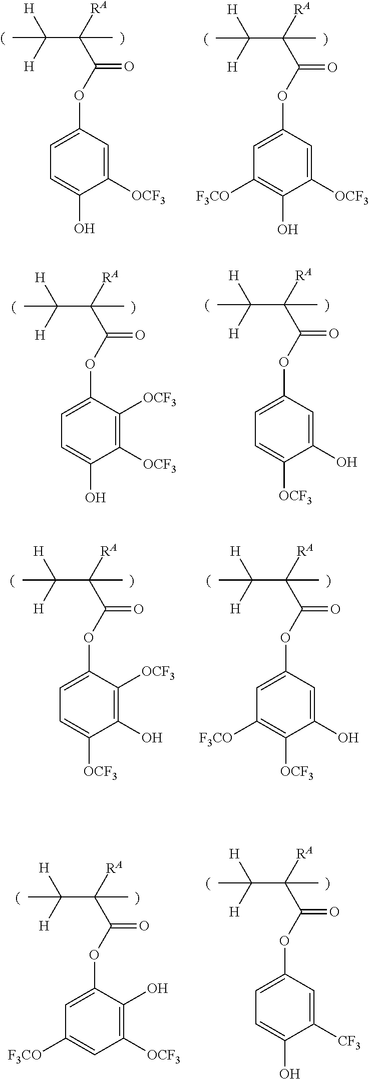

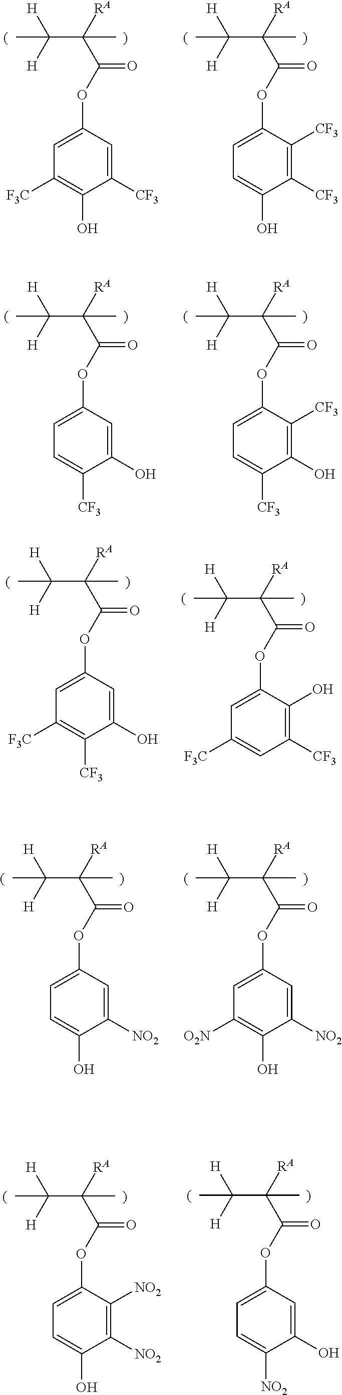

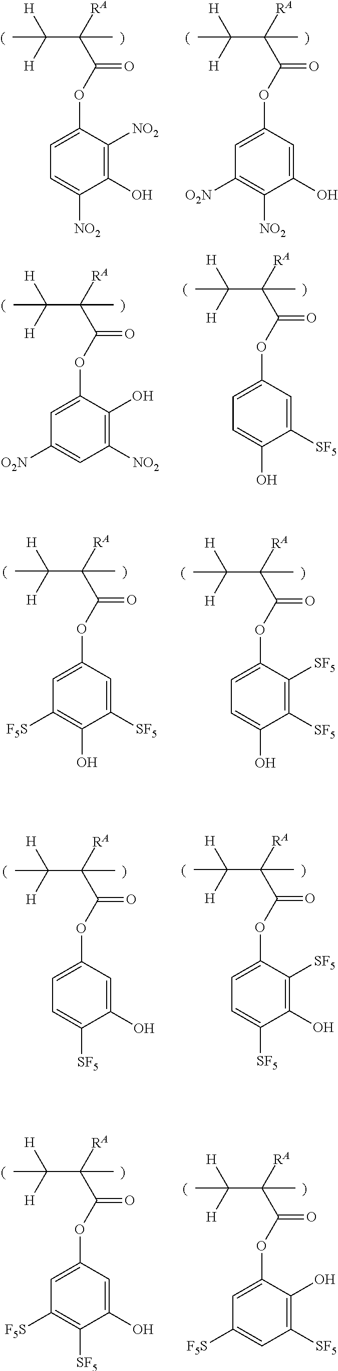

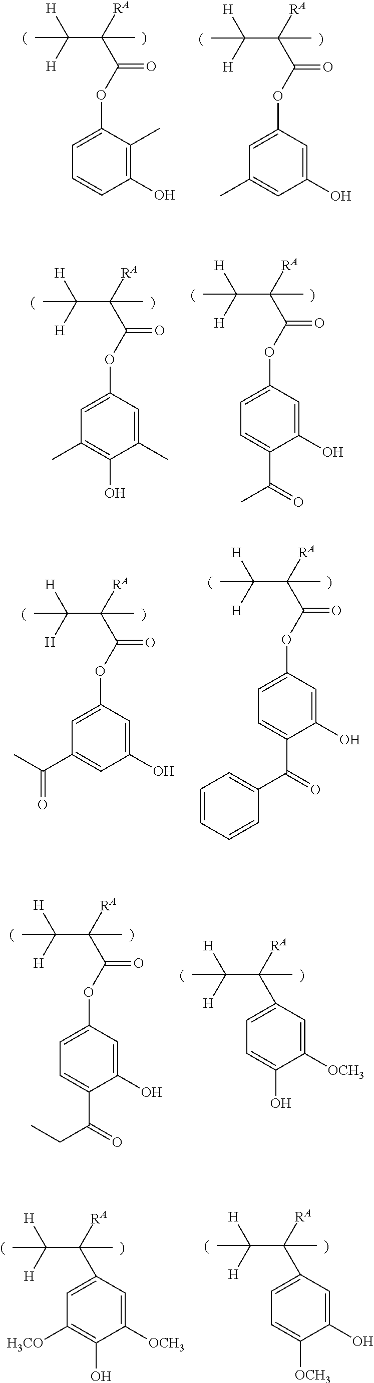

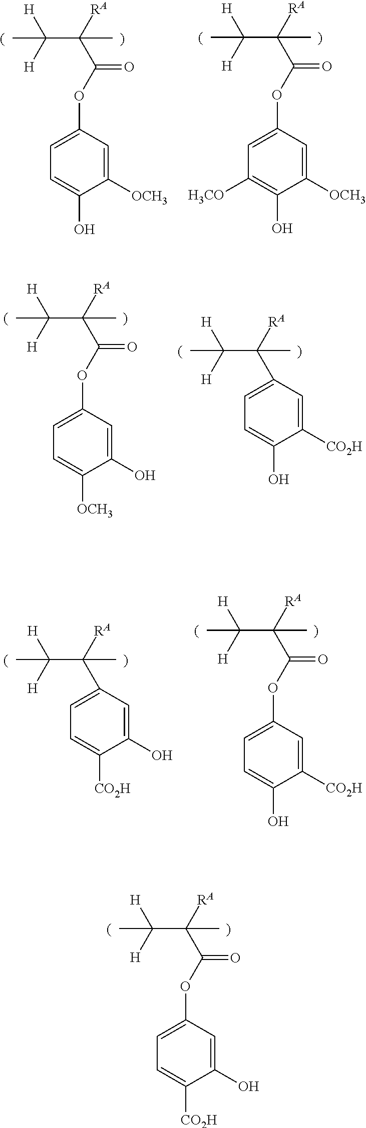

The repeat unit b1 or b2 is particularly preferably one having a lactone ring as a polar group in ArF lithography, and is particularly preferably one having a phenol moiety in KrF lithography, EB lithography, and EUV lithography.

The polymer may contain at least one selected from a repeat unit having the following formula (c1) (hereinafter, also referred to as a repeat unit c1), a repeat unit having the following formula (c2) (hereinafter, also referred to as a repeat unit c2), a repeat unit having the following formula (c3) (hereinafter, also referred to as a repeat unit c3), a repeat unit having the following formula (c4) (hereinafter, also referred to as a repeat unit c4), and a repeat unit having the following formula (c5) (hereinafter, also referred to as a repeat unit c5).

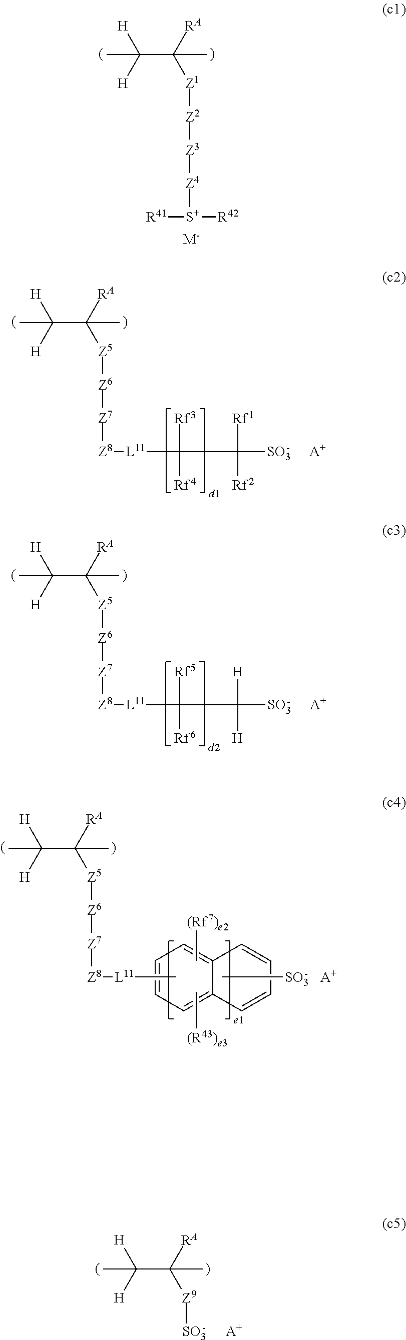

In the formulas (c1) to (c5), R^{A}s are each independently a hydrogen atom, a fluorine atom, a methyl group, or a trifluoromethyl group. Z¹ is a single bond or a phenylene group which may have a substituent. Z² is a single bond, **—C(═O))O—Z²¹—, **—C(═O)—N(H)—Z²¹—, or **—O—Z²¹—. Z²¹ is a C₁-C₆ aliphatic hydrocarbylene group, a phenylene group, or a divalent group obtained by combining these groups, and may contain a halogen atom, a carbonyl group, an ester bond, an ether bond, or a hydroxy group. Z³ is a single bond, an ether bond, an ester bond, a sulfonate bond, an amide bond, a sulfonamide bond, a carbonate bond, or a carbamate bond. Z⁴ is a single bond, or a C₁-C₆ aliphatic hydrocarbylene group, a phenylene group, or a divalent group obtained by combining these groups, and may contain a halogen atom, a carbonyl group, an ester bond, an ether bond, or a hydroxy group. Z⁵s are each independently a single bond, a phenylene group which may contain a substituent, a naphthylene group which may contain a substituent, *—C(═O)—O—Z⁵¹—, or *—C(═O)—N(R)—Z⁵¹—. Z⁵¹ is a C₁-C₁₀ aliphatic hydrocarbylene group, a phenylene group, or a naphthylene group, and the aliphatic hydrocarbylene group may contain a halogen atom, a hydroxy group, an ether bond, an ester bond, or a lactone ring. R is a hydrogen atom or a C₁-C₁₀ hydrocarbyl group which may contain a heteroatom.

Z⁶ is a single bond, an ether bond, an ester bond, a sulfonate bond, an amide bond, a sulfonamide bond, a carbonate bond, or a carbamate bond. Z⁷s are each independently a single bond, ***—Z⁷¹—C(═O)—O—, ***—C(═O)—N(R)—Z⁷¹—, or ***—O—Z⁷¹—. Z⁷¹ is a C₁-C₂₀ hydrocarbylene group which may contain a heteroatom. R is as defined above. Z⁸s are each independently a single bond, ****—Z⁸¹—C(═O)—O—, ****—C(═O)—N(R)—Z⁸¹—, or ****—O—Z⁸¹—. Z⁸¹ is a C₁-C₂₀ hydrocarbylene group which may contain a heteroatom. R is as defined above. Z⁹ is a single bond, a methylene group, an ethylene group, a phenylene group, a fluorinated phenylene group, a phenylene group replaced with a trifluoromethyl group, *—C(O)O—O—Z⁹¹—, *—C(═O)—N(R)—Z⁹¹—, or *—O—Z⁹¹. Z⁹¹ is a C₁-C₆ aliphatic hydrocarbylene group, a phenylene group, a fluorinated phenylene group, or a phenylene group replaced with a trifluoromethyl group, and may contain a carbonyl group, an ester bond, an ether bond, or a hydroxy group. R is as defined above. * represents a bond with a carbon atom of a main chain. ** represents a bond with Z¹. *** represents a bond with Z⁶. **** represents a bond with Z⁷.

The aliphatic hydrocarbylene group of Z²¹, Z⁵¹, and Z⁹¹ may be linear, branched, or cyclic, and specific examples thereof include: an alkanediyl group such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-1,1-diyl group, a butane-1,2-diyl group, a butane-1,3-diyl group, a butane-2,3-diyl group, a butane-1,4-diyl group, a 1,1-dimethylethane-1,2-diyl group, a pentane-1,5-diyl group, a 2-methylbutane-1,2-diyl group, or a hexane-1,6-diyl group; a cycloalkanediyl group such as a cyclopropanediyl group, a cyclobutanediyl group, a cyclopentanediyl group, or a cyclohexanediyl group; and a group obtained by combining these groups.

The hydrocarbylene group of Z⁷¹ and Z⁸¹ which may contain a heteroatom may be saturated or unsaturated, and may be linear, branched, or cyclic. Specific examples thereof include, but are not limited to, those listed below.

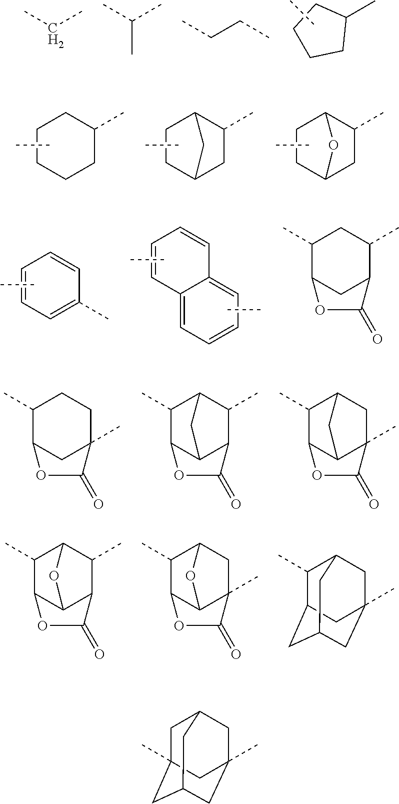

wherein the dashed line represents a bond.

In the formula (c1), R⁴¹ and R⁴² are each independently a C₁-C₂₀ hydrocarbyl group which may contain a heteroatom. The hydrocarbyl group may be saturated or unsaturated, and may be linear, branched, or cyclic. Specific examples thereof include: a C₁-C₂₀ alkyl group such as a methyl group, an ethyl group, a n-propyl group, an isopropyl group, a n-butyl group, an isobutyl group, a sec-butyl group, or a tert-butyl group; a C₃-C₂₀ cyclic saturated hydrocarbyl group such as a cyclopropyl group, a cyclopentyl group, a cyclohexyl group, a cyclopropylmethyl group, a 4-methylcyclohexyl group, a cyclohexylmethyl group, a norbornyl group, or an adamantyl group; a C₂-C₂₀ alkenyl group such as a vinyl group, a 1-propenyl group, a 2-propenyl group, a butenyl group, or a hexenyl group; a C₃-C₂₀ cyclic unsaturated hydrocarbyl group such as a cyclohexenyl group; a C₆-C₂₀ aryl group such as a phenyl group, a naphthyl group, or a thienyl group; a C₇-C₂₀ aralkyl group such as a benzyl group, a 1-phenylethyl group, or a 2-phenylethyl group; and a group obtained by combining these groups, and an aryl group is preferable. Some or all of the hydrogen atoms of the hydrocarbyl group may be replaced with a group containing a heteroatom such as an oxygen atom, a sulfur atom, a nitrogen atom, or a halogen atom, some of the (—CH₂-)s in the hydrocarbyl group may be replaced with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

R⁴¹ and R⁴² may be bonded to each other to form a ring together with a sulfur atom to which they are bonded. At this time, specific examples of the ring include those having the following formulas:

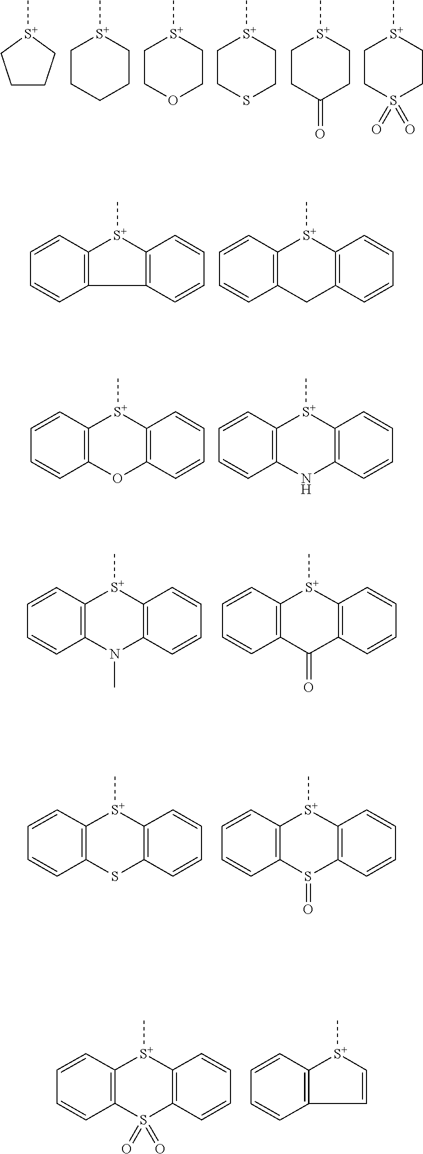

wherein the dashed line is a bond with Z⁴.

Specific examples of the cation of the repeat unit c1 include, but are not limited to, those listed below. Note that, in the following formulas, R^{A} is as defined above.

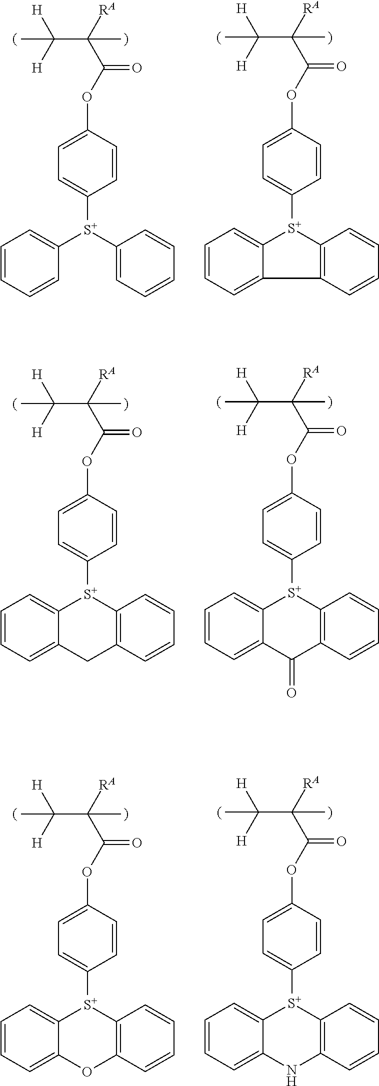

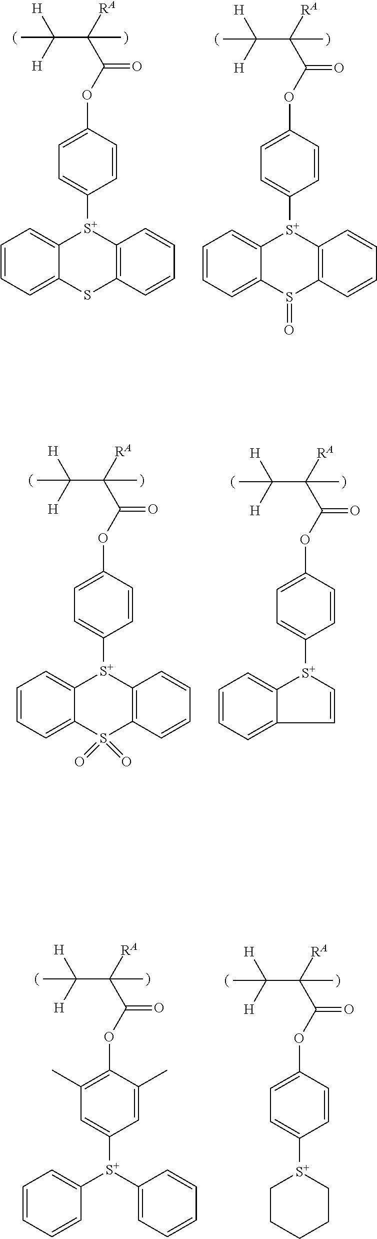

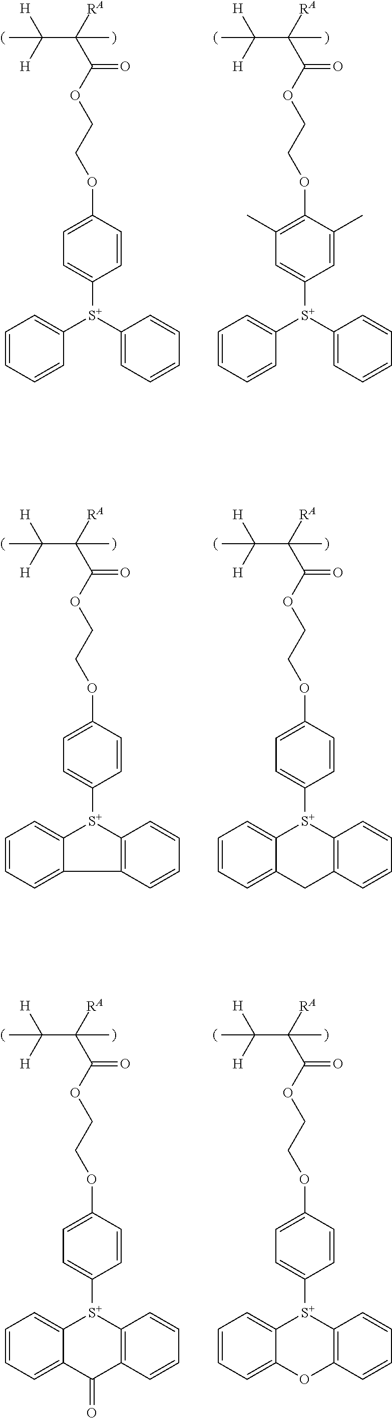

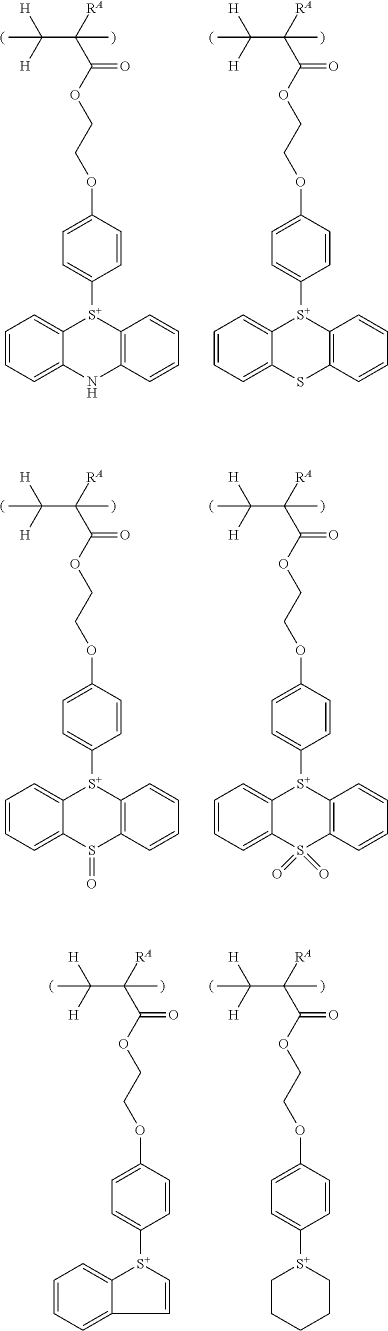

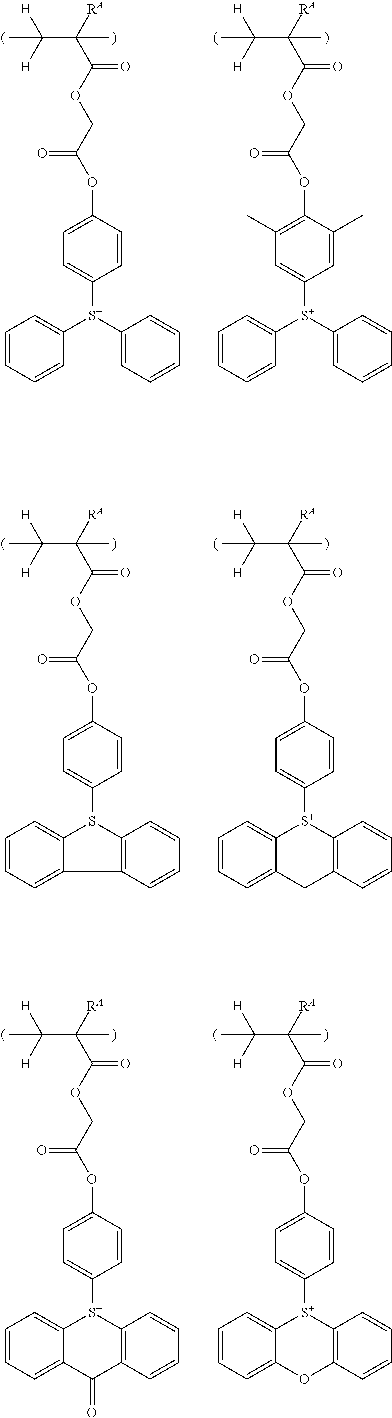

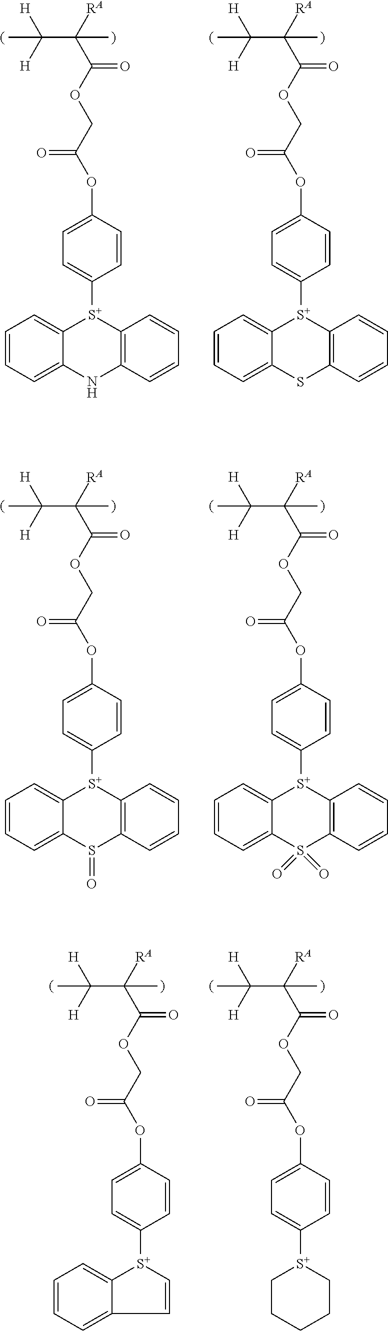

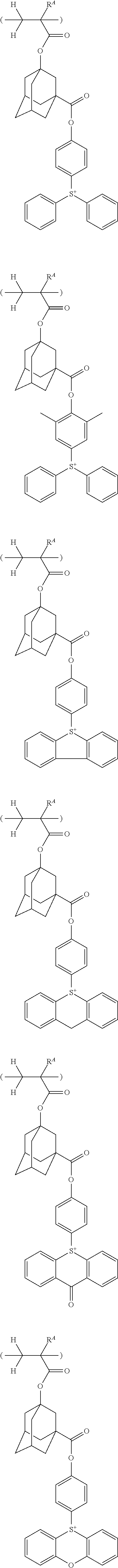

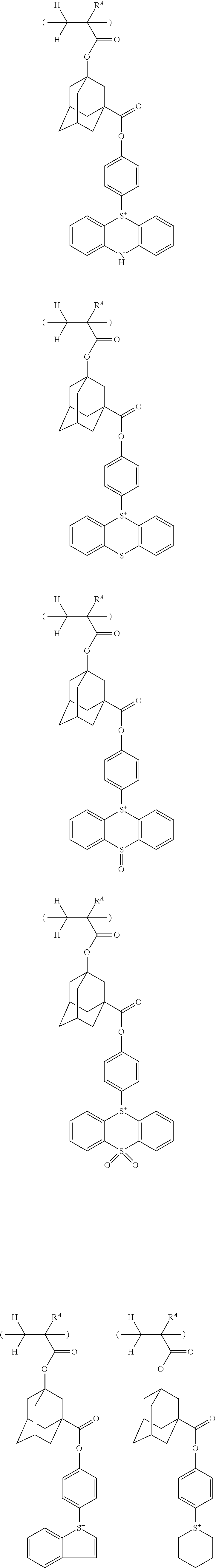

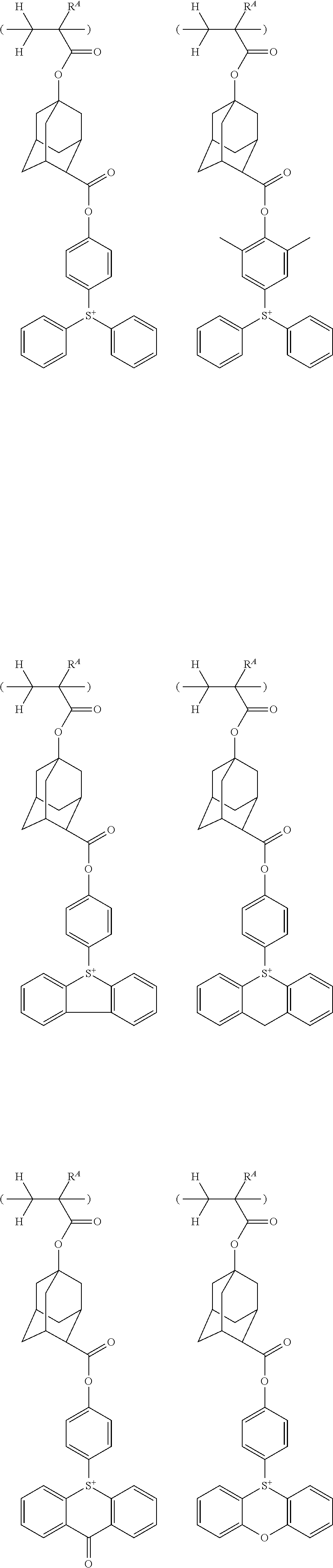

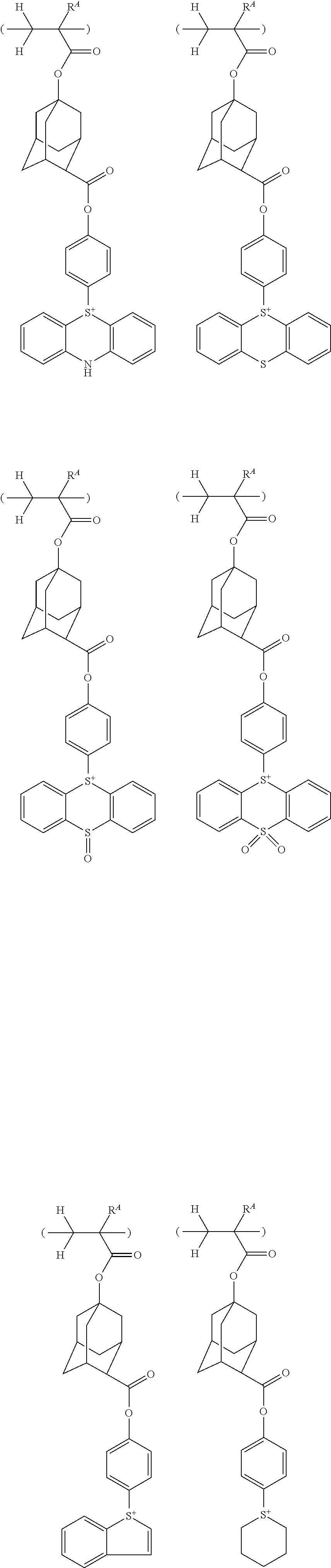

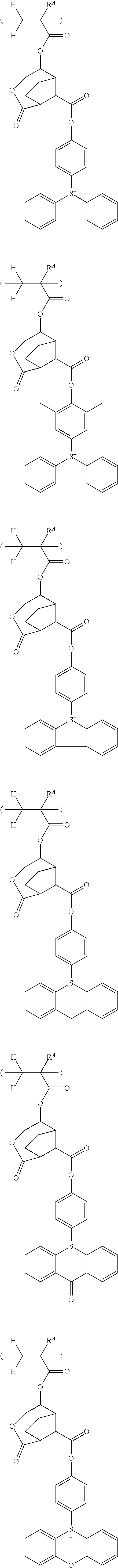

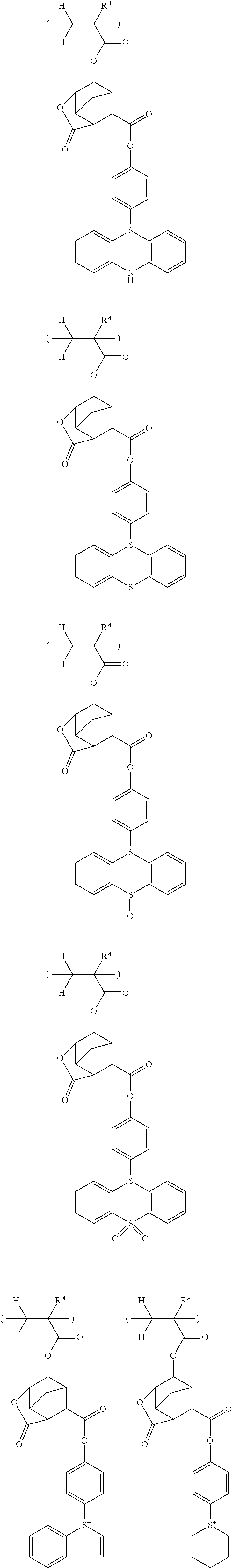

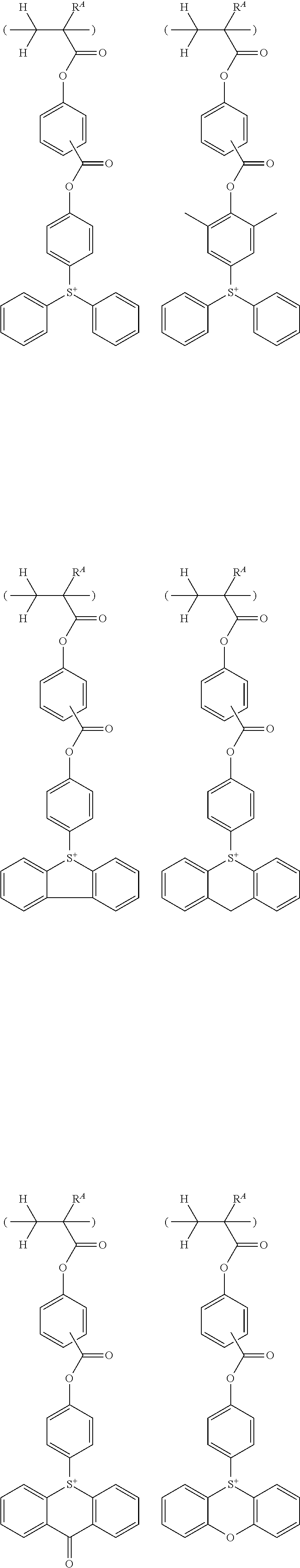

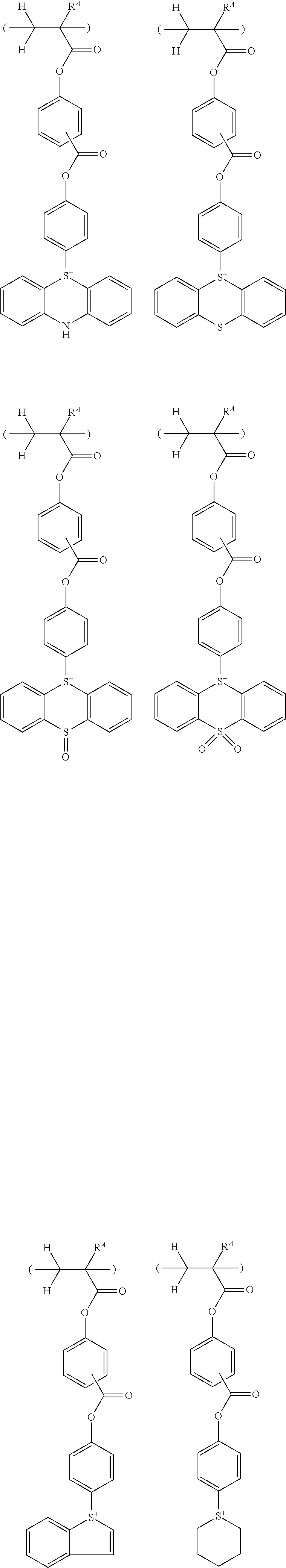

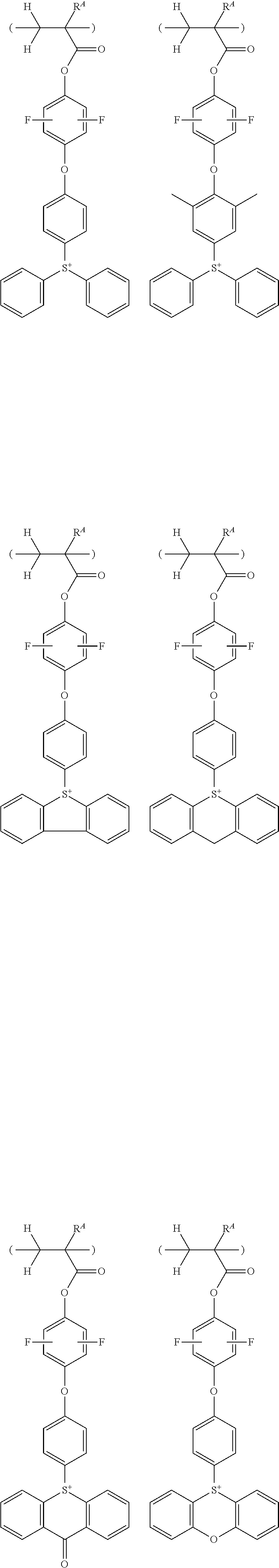

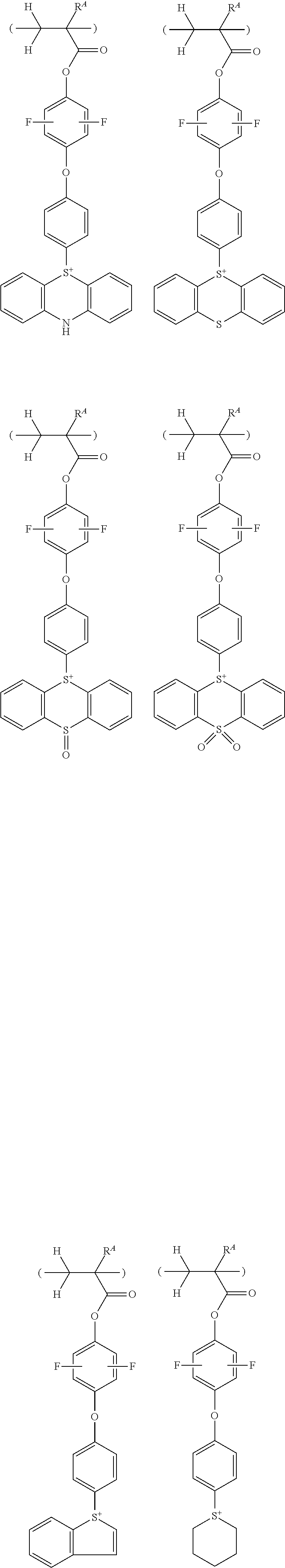

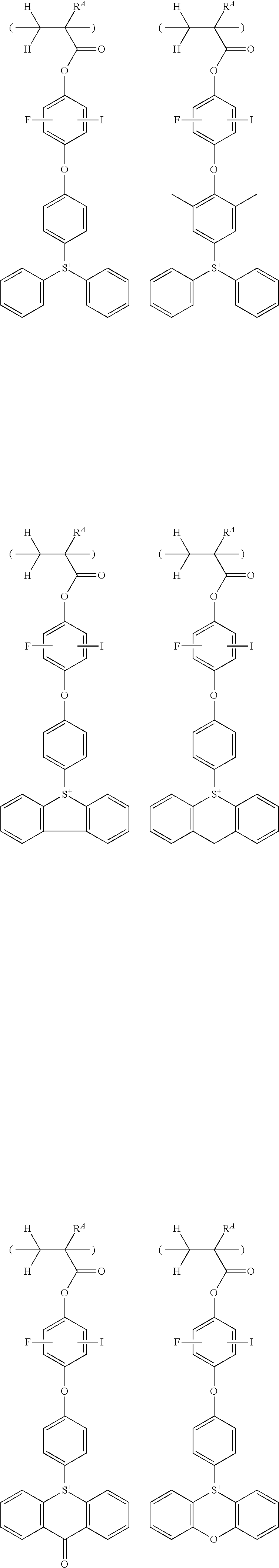

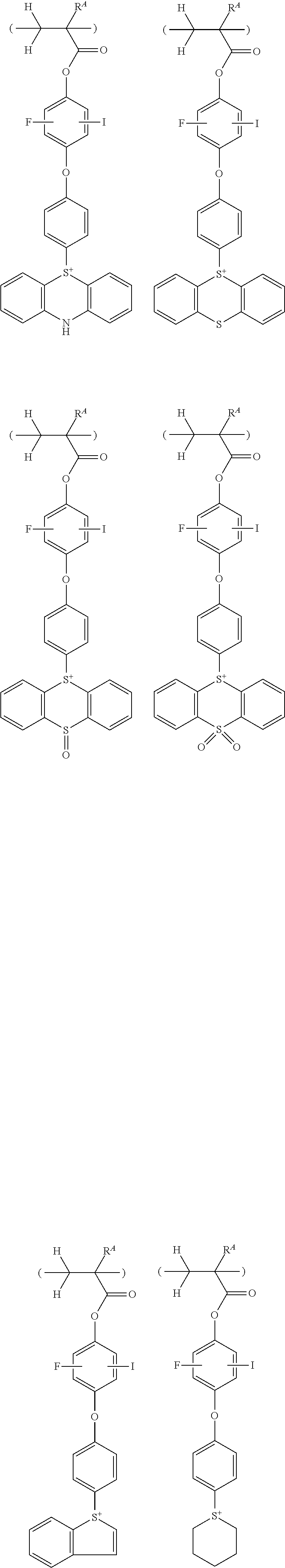

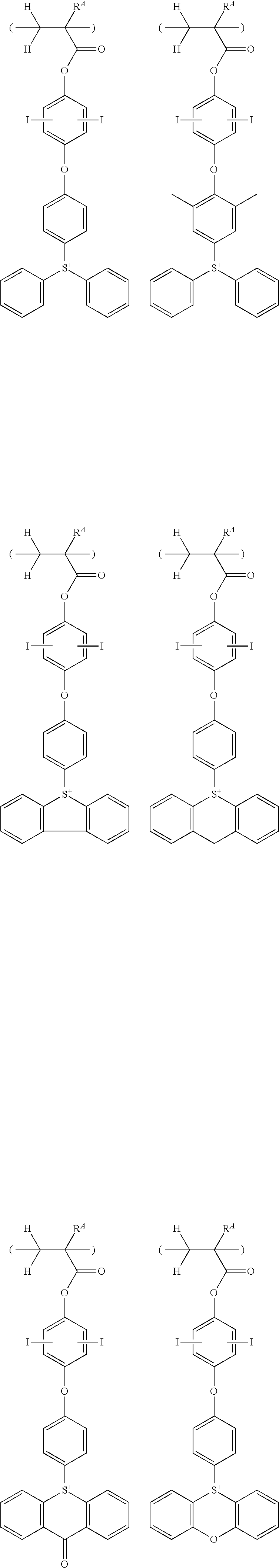

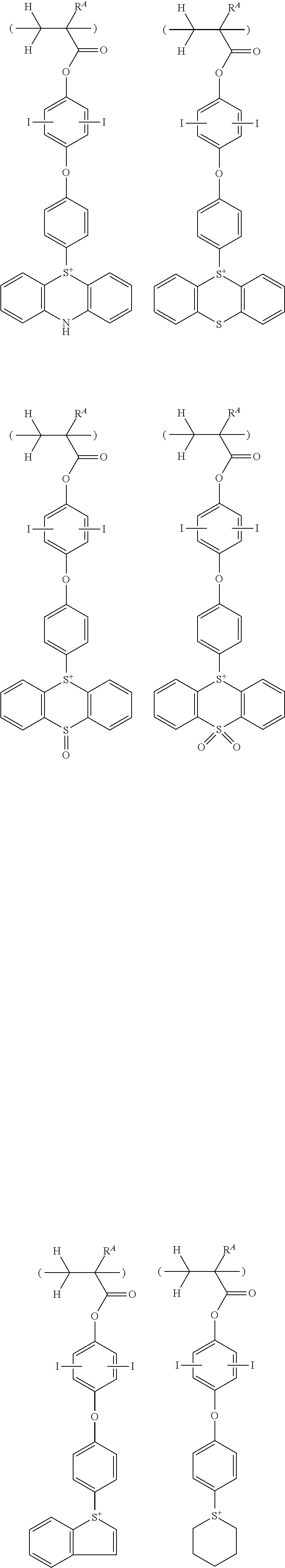

In the formula (c1), M⁻ is a non-nucleophilic counter ion. The non-nucleophilic counter ion is preferably a halide ion, a sulfonate anion, an imidic acid anion, or a methide acid anion. Specific examples of the halide ion include a chloride ion and a bromide ion. Specific examples of the sulfonic acid anion (sulfonate ion) include: a fluoroalkyl sulfonate ion such as a triflate ion, a 1,1,1-trifluoroethane sulfonate ion, or a nonafluorobutane sulfonate ion; an aryl sulfonate ion such as a tosylate ion, a benzene sulfonate ion, a 4-fluorobenzenesulfonate ion, or a 1,2,3,4,5-pentafluorobenzenesulfonate ion; and an alkyl sulfonate ion such as a mesylate ion or a butanesulfonate ion. Specific examples of the imidic acid anion (imide ion) include a bis(trifluoromethylsulfonyl) imide ion, a bis(perfluoroethylsulfonyl) imide ion, and a bis(perfluorobutylsulfonyl) imide ion. Specific examples of the methide acid anion (methide ion) include a tris(trifluoromethylsulfonyl) methide ion and a tris(perfluoroethylsulfonyl) methide ion.

Other examples of the non-nucleophilic counter ion include an anion having any one of the formulas (c1-1) to (c1-4).

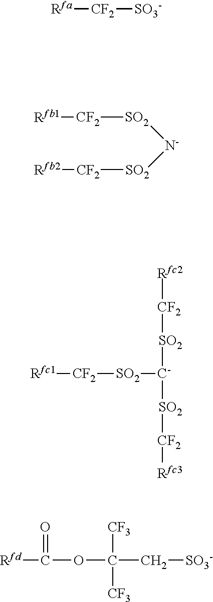

In the formula (c1-1), R^{fa} is a fluorine atom or a C₁-C₄₀ hydrocarbyl group which may contain a heteroatom. The hydrocarbyl group may be saturated or unsaturated, and may be linear, branched, or cyclic. Specific examples thereof include those similar to those exemplified as the hydrocarbyl group of R^{fa1} in the formula (c1-1-1) described below. Some or all of the hydrogen atoms of the hydrocarbyl group may be replaced with a group containing a heteroatom such as an oxygen atom, a sulfur atom, a nitrogen atom, or a halogen atom, some of the (—CH₂-)s in the hydrocarbyl group may be replaced with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a nitro group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

The anion having the formula (c1-1) preferably has the following formula (c1-1-1):

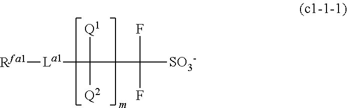

In the formula (c1-1-1), Q¹ and Q² are each independently a hydrogen atom, a fluorine atom, or a C₁-C₆ fluorinated saturated hydrocarbyl group, but at least one of Q¹ and Q² is preferably a trifluoromethyl group for improving solvent solubility. m is 0, 1, 2, 3, or 4, and particularly preferably 1. R^{fa1} is a C₁-C₄₀ hydrocarbyl group which may contain a heteroatom. The heteroatom is preferably an oxygen atom, a nitrogen atom, a sulfur atom, a halogen atom, or the like, and more preferably an oxygen atom. The hydrocarbyl group particularly preferably has 6 to 30 carbon atoms from a viewpoint of obtaining high resolution in fine pattern formation.

In the formula (Xa-1-1), the C₁-C₄₀ hydrocarbyl group of R^{fa1} may be saturated or unsaturated, and may be linear, branched, or cyclic. Specific examples thereof include: a C₁-C₄₀ alkyl group such as a methyl group, an ethyl group, a n-propyl group, an isopropyl group, a n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, or an icosyl group; a C₃-C₄₀ cyclic saturated hydrocarbyl group such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, or a dicyclohexylmethyl group; a C₂-C₄₀ unsaturated aliphatic hydrocarbyl group such as a 2-propenyl group or a 3-cyclohexenyl group; a C₆-C₄₀ aryl group such as a phenyl group, a 1-naphthyl group, a 2-naphthyl group, or a 9-fluorenyl group; a C₇-C₄₀ aralkyl group such as a benzyl group or a diphenylmethyl group; a C₇-C₄₀ aromatic ring-containing polycyclic hydrocarbyl group such as a 9,10-ethano-9,10-dihydroanthryl group or a 6,13-ethano-6,13-dihydropentacenyl group; a C₁₇-C₄₀ hydrocarbyl group having a steroid skeleton; and a group obtained by combining these groups.

Some or all of the hydrogen atoms of the hydrocarbyl group may be replaced with a group containing a heteroatom such as an oxygen atom, a sulfur atom, a nitrogen atom, or a halogen atom, some of the (—CH₂-)s in the hydrocarbyl group may be replaced with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a nitro group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like. Specific examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy) methyl group, an acetoxymethyl group, a 2-carboxy-1-cyclohexyl group, a 2-oxopropyl group, a 4-oxo-1-adamantyl group, and a 3-oxocyclohexyl group.

In the formula (c1-1-1), L^{a1} is a single bond, an ether bond, an ester bond, a sulfonate bond, a carbonate bond, or a carbamate bond, but is preferably an ether bond or an ester bond, and more preferably an ester bond from a viewpoint of synthesis.

Specific examples of the anion having the formula (c1-1) include, but are not limited to, those listed below. Note that, in the following formulas, Q¹ is as defined above, and Ac is an acetyl group.

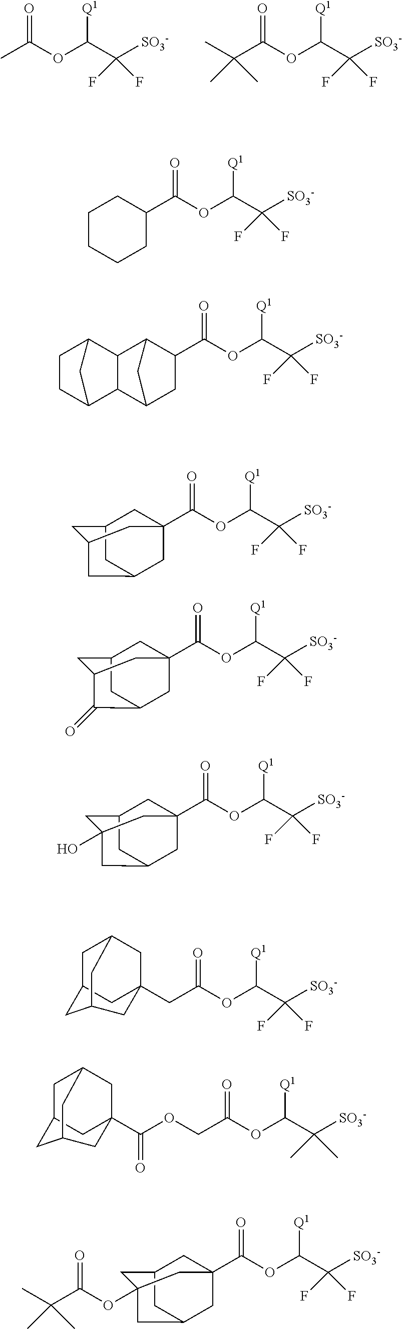

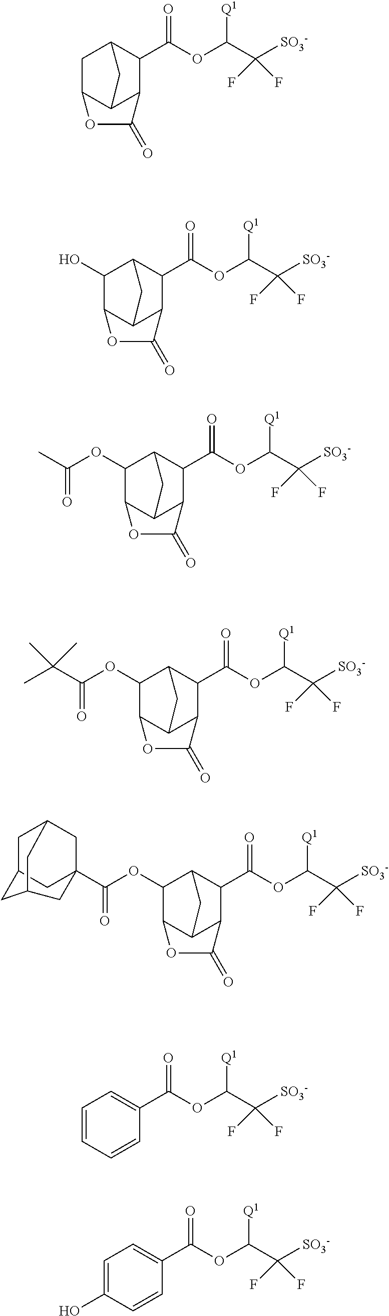

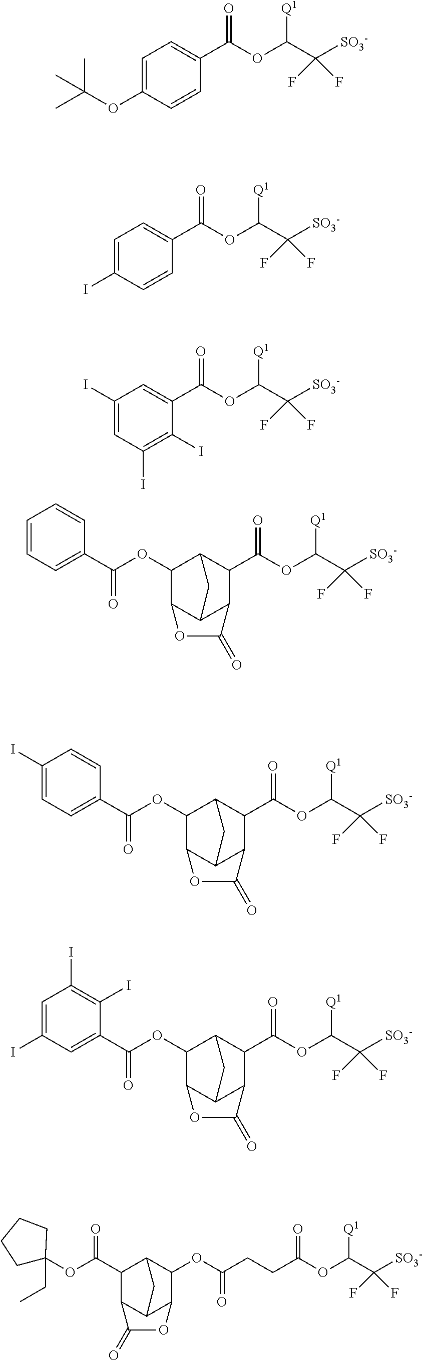

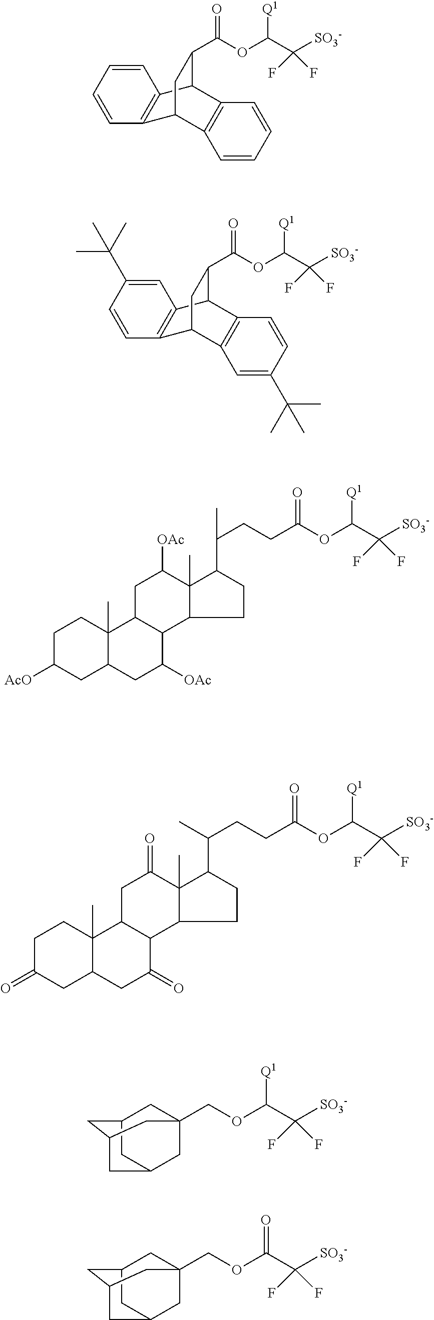

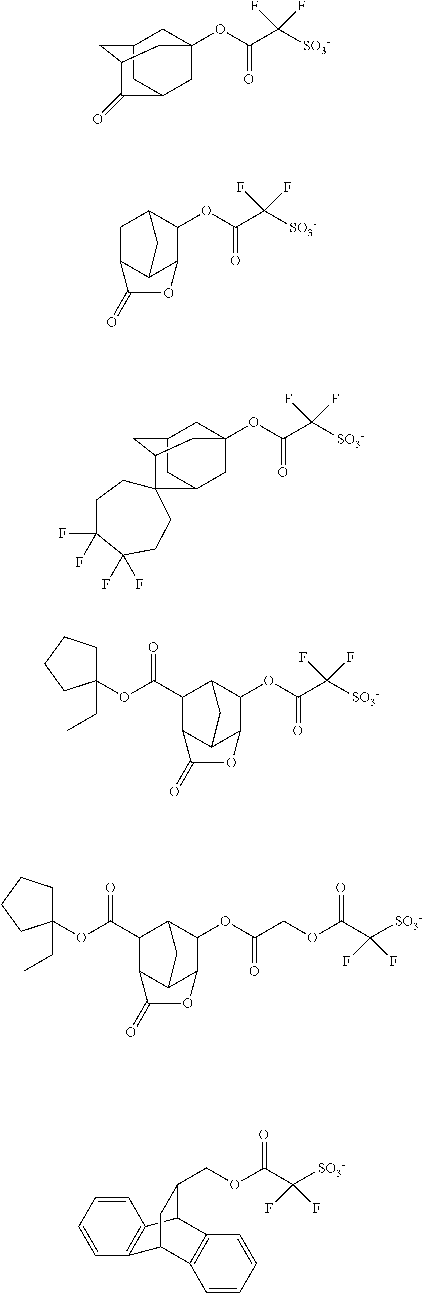

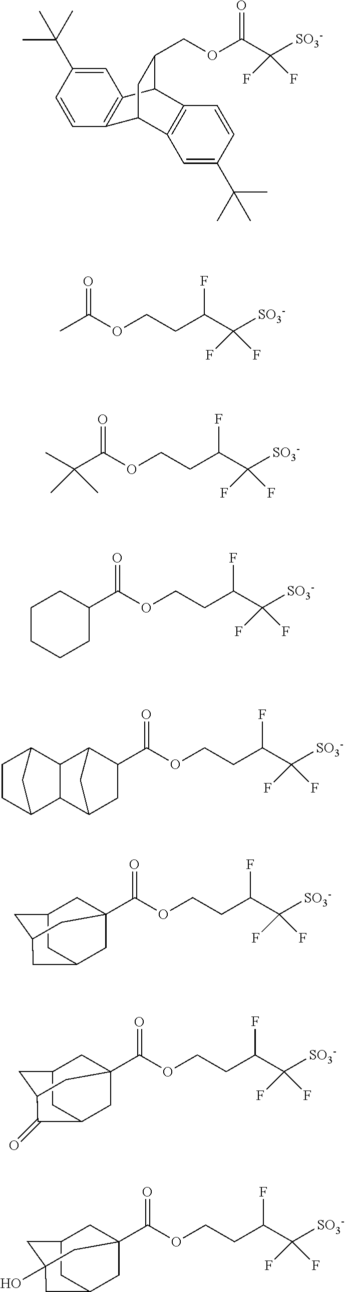

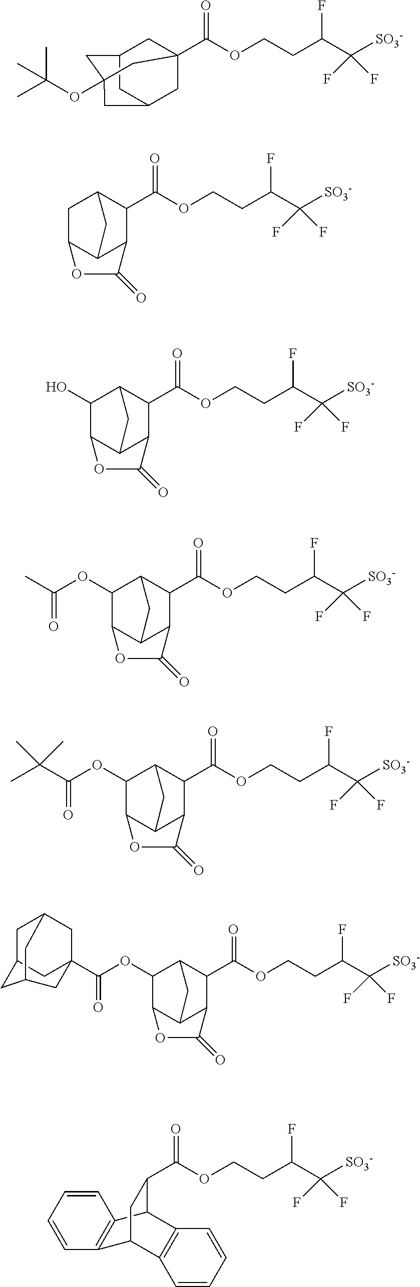

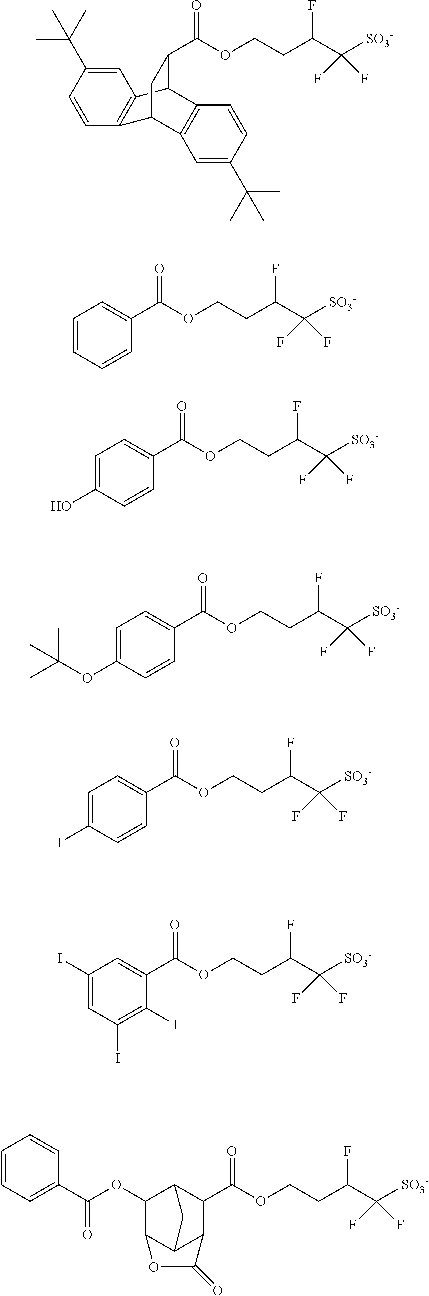

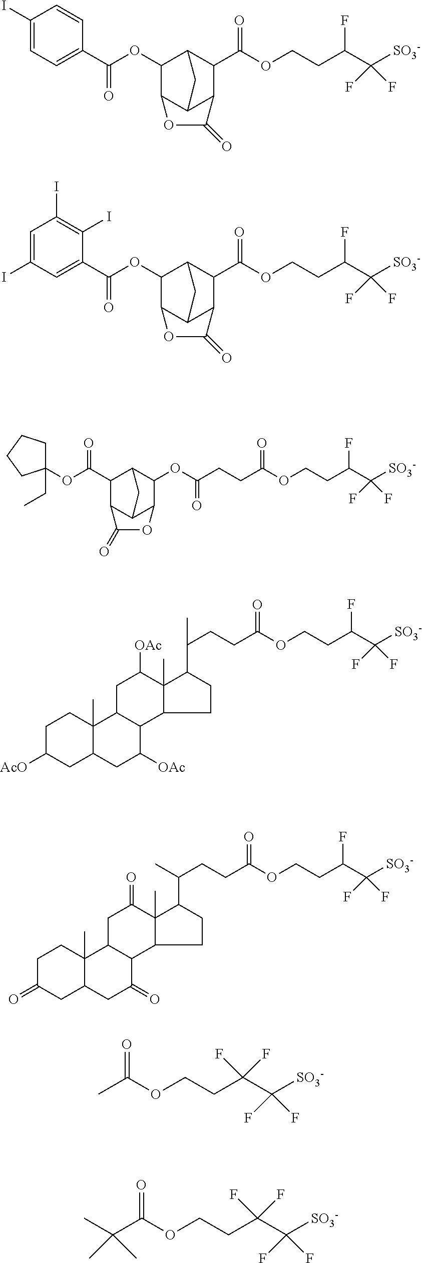

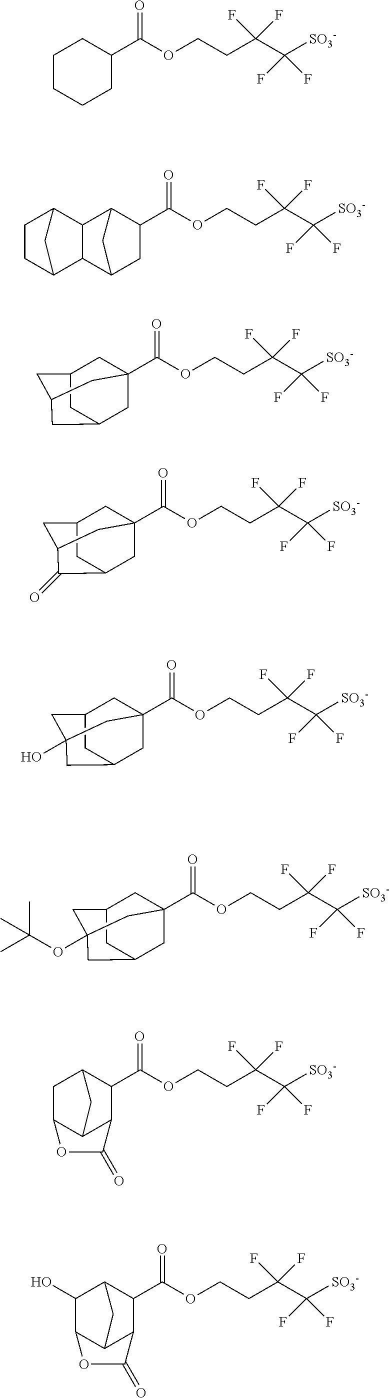

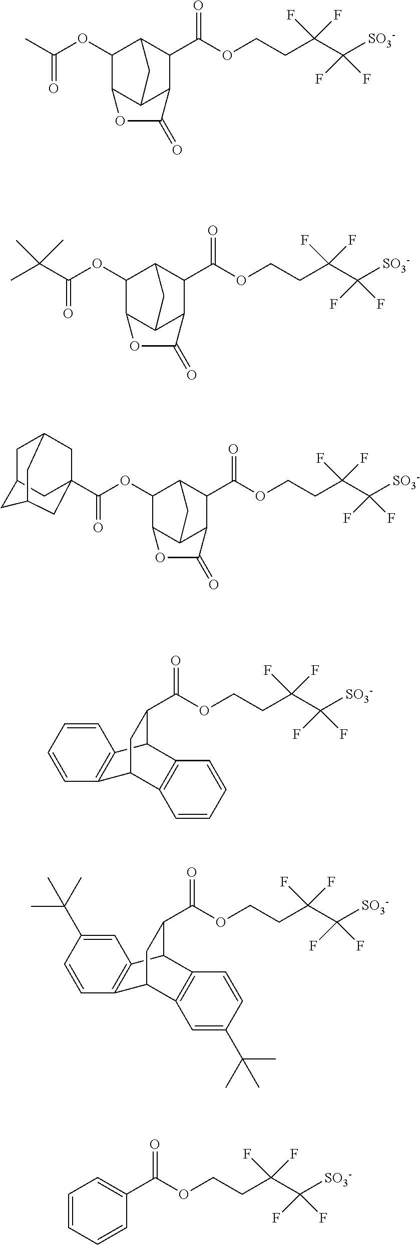

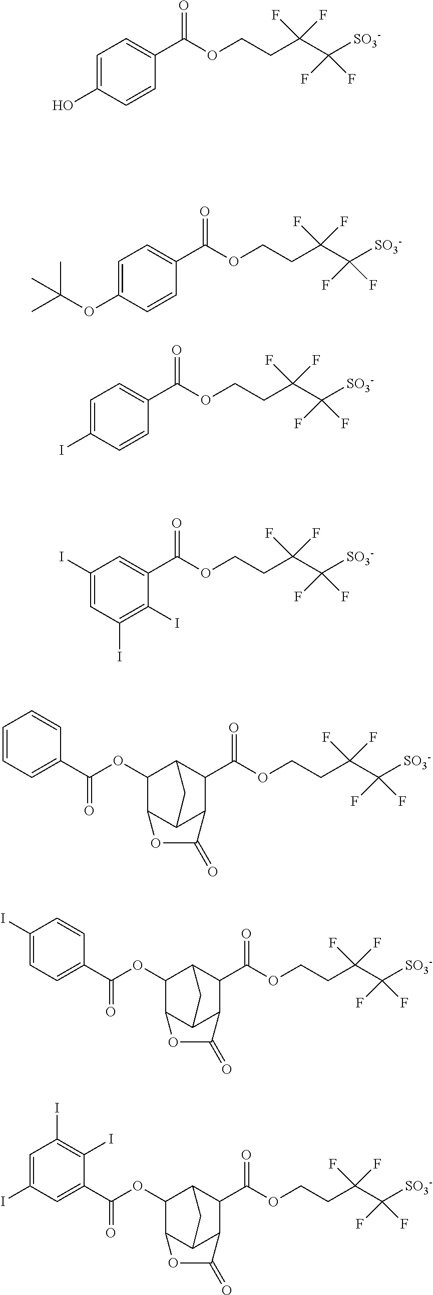

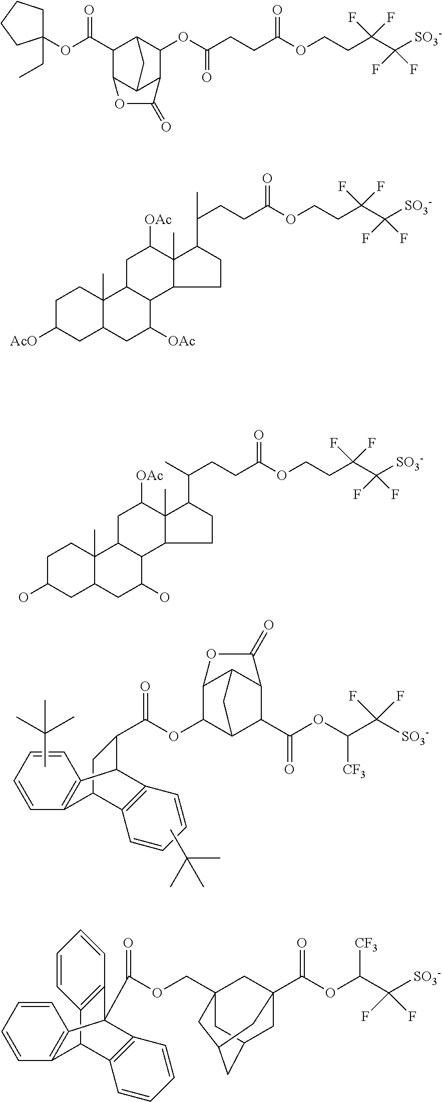

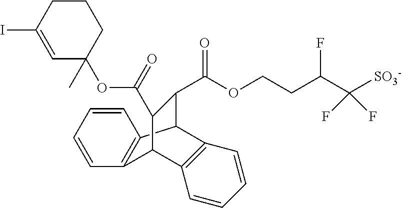

Other specific examples of the anion having the formula (c1-1) include those described in paragraphs [0042] to [0049] of JP-A 2025-23601, paragraphs [0015] to [0017] of JP-A 2021-70692, paragraphs [0145] and [0148] of JP 7091762, paragraphs [0022] and [0023] of JP 7628783, and paragraph [0224] of JP 7109178.

In the formula (c1-2), R^{fb1} and R^{fb2} are each independently a fluorine atom or a C₁-C₄₀ hydrocarbyl group which may contain a heteroatom. The hydrocarbyl group may be saturated or unsaturated, and may be linear, branched, or cyclic. Specific examples thereof include those similar to those exemplified as the hydrocarbyl group of R^{fa1} in the formula (c1-1-1). R^{fb1} and R^{fb2} are each preferably a fluorine atom or a linear C₁-C₄ fluorinated alkyl group. R^{fb1} and R^{fb2} may be bonded to each other to form a ring together with a group to which they are bonded (—CF₂—SO₂—N⁻—SO₂—CF₂—), and in this case, a group obtained by bonding of R^{fb1} and R^{fb2} is preferably a fluorinated ethylene group or a fluorinated propylene group.

In the formula (c1-3), R^{fc1}, R^{fc2}, and R^{fc3} are each independently a fluorine atom or a C₁-C₄₀ hydrocarbyl group which may contain a heteroatom. The hydrocarbyl group may be saturated or unsaturated, and may be linear, branched, or cyclic. Specific examples thereof include those similar to those exemplified as the hydrocarbyl group of R^{fa1} in the formula (c1-1-1). R^{fc1}, R^{fc2}, and R^{fc3} are each preferably a fluorine atom or a linear C₁-C₄ fluorinated alkyl group. R^{fc1} and R^{fc2} may be bonded to each other to form a ring together with a group to which they are bonded (—CF₂—SO₂—C⁻—SO₂—CF₂—), and in this case, a group obtained by bonding of R^{fc1} and R^{fc2} is preferably a fluorinated ethylene group or a fluorinated propylene group.

In the formula (c1-4), R^{fd} is a C₁-C₄₀ hydrocarbyl group which may contain a heteroatom. The hydrocarbyl group may be saturated or unsaturated, and may be linear, branched, or cyclic. Specific examples thereof include those similar to those exemplified as the hydrocarbyl group of R^{fa1} in the formula (c1-1-1). Some or all of the hydrogen atoms of the hydrocarbyl group may be replaced with a group containing a heteroatom such as an oxygen atom, a sulfur atom, a nitrogen atom, or a halogen atom, some of the (—CH₂-)s in the hydrocarbyl group may be replaced with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a nitro group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

Specific examples of the anion having the formula (c1-4) include, but are not limited to, those listed below.

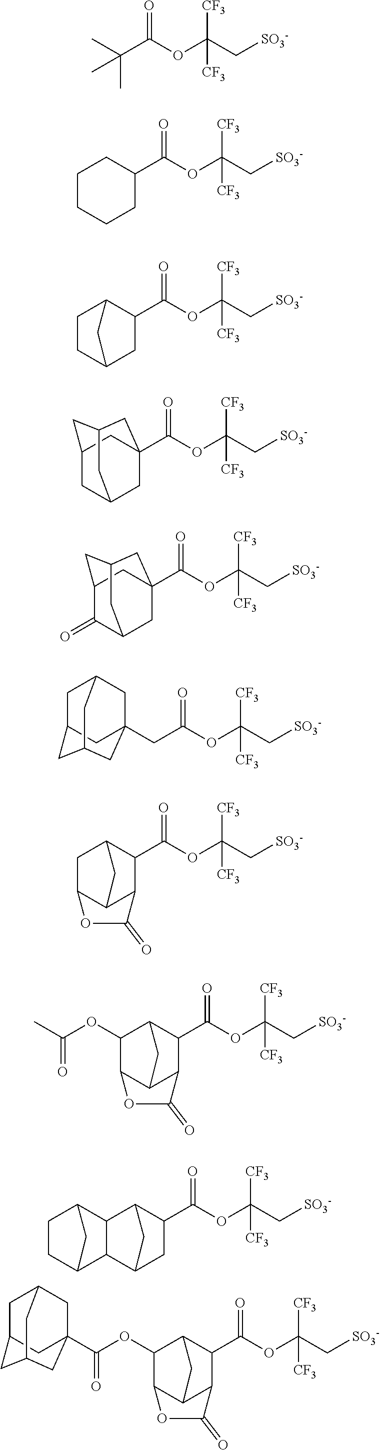

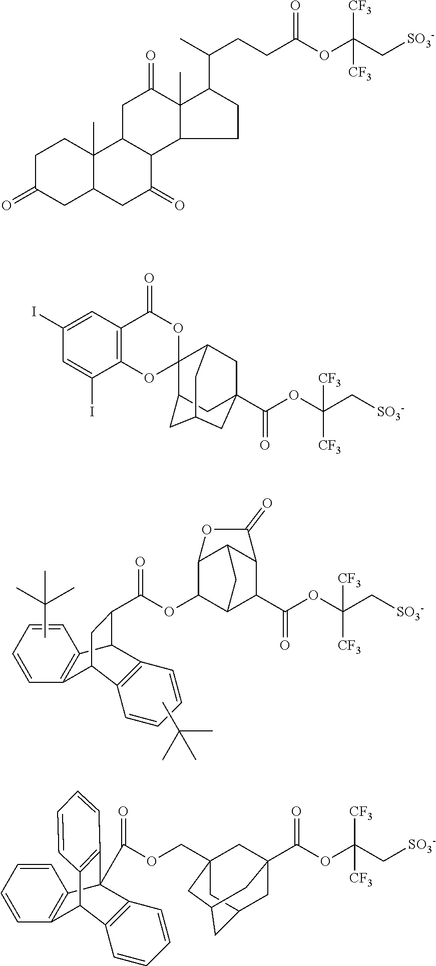

Other specific examples of the anion having the formula (c1-4) include those described in paragraphs [0050] to [0058] of JP-A 2025-23601.

Examples of the non-nucleophilic counter ion further include an anion having an aromatic ring with an iodine atom or a bromine atom as a substituent. Specific examples of such an anion include those having the following formula (c1-5).

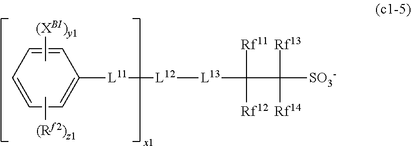

In the formula (c1-5), x1 is 1, 2, or 3. y1 is 1, 2, 3, 4, or 5. z1 is 0, 1, 2, or 3. Provided that 1≤y1+z1≤5. y1 is preferably 1, 2, or 3, and more preferably 2 or 3. z1 is preferably 0, 1, or 2.

In the formula (c1-5), X^{BI} is an iodine atom or a bromine atom. When x1 and/or y1 is 2 or more, X^{BI}s may be the same as or different from each other.

In the formula (c1-5), L¹¹ is a single bond, an ether bond, an ester bond, a sulfonate bond, a carbonate bond, a carbamate bond, or a C₁-C₆ saturated hydrocarbylene group, and some of the (—CH₂-)s of the hydrocarbylene group may be replaced with an ether bond or an ester bond. The saturated hydrocarbylene group may be linear, branched, or cyclic.

In the formula (c1-5), L¹² is a single bond or a C₁-C₂₀ hydrocarbylene group which may contain a heteroatom when x1 is 1, and is a C₁-C₂₀ (x1+1)-valent hydrocarbon group which may contain a heteroatom when x1 is 2 or 3.

The C₁-C₂₀ hydrocarbylene group of L¹² may be saturated or unsaturated, and may be linear, branched, or cyclic. Specific examples thereof include: a C₁-C₂₀ alkanediyl group such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, or a dodecane-1,12-diyl group; a C₃-C₂₀ cyclic saturated hydrocarbylene group such as a cyclopentanediyl group, a cyclohexanediyl group, a norbornanediyl group, an adamantanediyl group, or a tricyclo [5.2.1.0^{2,6}] decanediyl group; a C₂-C₂₀ unsaturated aliphatic hydrocarbylene group such as vinylene group or a propene-1,3-diyl group; a C₆-C₂₀ arylene group such as a phenylene group, a naphthylene group, or an anthracenediyl group; a C₇-C₂₀ aromatic ring-containing polycyclic hydrocarbylene group such as a 9,10-ethano-9,10-dihydroanthracenediyl group or a 6,13-ethano-6,13-dihydropentacene group; and a group obtained by combining these groups. The C₁-C₂₀ (x1+1)-valent hydrocarbon group of L¹² may be saturated or unsaturated, and may be linear, branched, or cyclic. Specific examples thereof include groups obtained by further removing one or two hydrogen atoms from the above-described specific examples of the C₁-C₂₀ hydrocarbylene group.

Some or all of the hydrogen atoms of the hydrocarbylene group and the (x1+1)-valent hydrocarbon group may be replaced with a group containing a heteroatom such as an oxygen atom, a sulfur atom, a nitrogen atom, or a halogen atom, some of the (—CH₂-)s in the hydrocarbylene group and the (x1+1)-valent hydrocarbon group may be replaced with a group containing a heteroatom such as an oxygen atom, a sulfur atom, or a nitrogen atom, and as a result, the hydrocarbylene group and the (x1+1)-valent hydrocarbon group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

In the formula (c1-5), L¹³ is a single bond, an ether bond, an ester bond, a sulfonate bond, a carbonate bond, or a carbamate bond.

In the formula (c1-5), R^{fe} is a hydroxy group, a carboxy group, a fluorine atom, a chlorine atom, a bromine atom, an amino group, a C₁-C₂₀ hydrocarbyl group, a C₁-C₂₀ hydrocarbyloxy group, a C₁-C₂₀ hydrocarbylthio group, a C₂-C₂₀ hydrocarbylcarbonyl group, a C₂-C₂₀ hydrocarbyloxycarbonyl group, a C₂-C₂₀ hydrocarbylcarbonyloxy group, a C₁-C₂₀ hydrocarbylsulfonyloxy group, —N(R^{feA})(R^{feB}), —N(R^{feC})—C(═O)—R^{feD}, or —N(R^{feC})—C(═O)—O—R^{feD}, the hydrocarbyl group, the hydrocarbyloxy group, the hydrocarbylthio group, the hydrocarbylcarbonyl group, the hydrocarbyloxycarbonyl group, the hydrocarbylcarbonyloxy group, and the hydrocarbylsulfonyloxy group may contain at least one selected from a fluorine atom, a chlorine atom, a bromine atom, an iodine atom, a hydroxy group, an amino group, an ester bond, and an ether bond. R^{feA} and R^{feB} are each independently a hydrogen atom or a C₁-C₆ saturated hydrocarbyl group. R^{feC} is a hydrogen atom or a C₁-C₆ saturated hydrocarbyl group, and may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{feD} is a C₁-C₁₆ aliphatic hydrocarbyl group, a C₆-C₁₂ aryl group, or a C₇-C₁₅ aralkyl group, and may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated, and may be linear, branched, or cyclic. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group may be linear, branched, or cyclic. When x1 and/or z1 are 2 or more, R^{fe}s may be the same as or different from each other.

Among these, R^{fe} is preferably a hydroxy group, —N(R^{feC})—C(═O)—R^{feD}, —N(R^{feC})—C(═O)—O—R^{feD}, a fluorine atom, a chlorine atom, a bromine atom, a methyl group, a methoxy group, or the like.

In the formula (c1-5), Rf¹¹ to Rf¹⁴ are each independently a hydrogen atom, a fluorine atom, or a trifluoromethyl group, and at least one of these is a fluorine atom or a trifluoromethyl group. Rf¹¹ and Rf¹² may be combined to form a carbonyl group. In particular, both Rf¹³ and Rf¹⁴ are preferably fluorine atoms.

Specific examples of the anion having the formula (c1-5) include, but are not limited to, those listed below. Note that, in the following formulas, X^{BI} is as defined above.

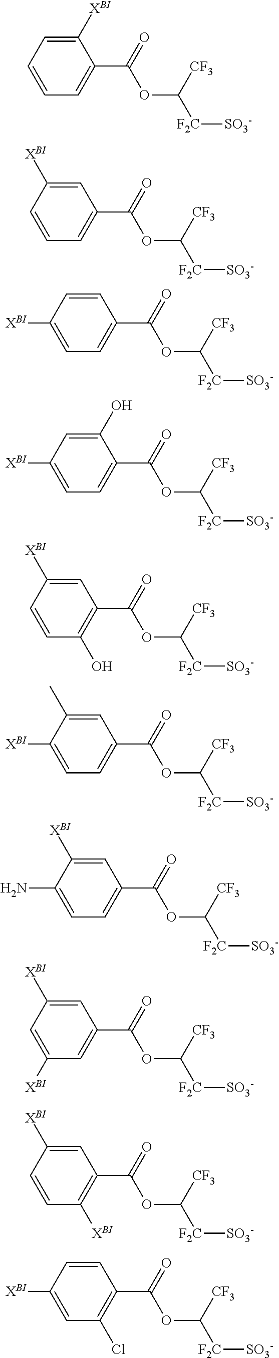

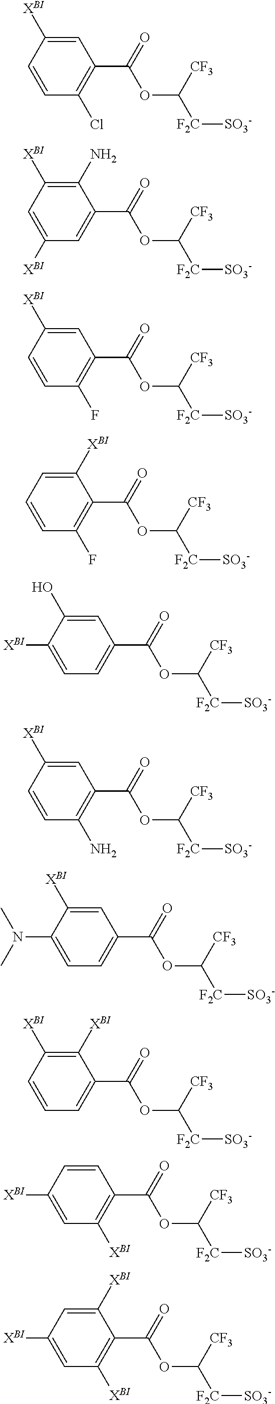

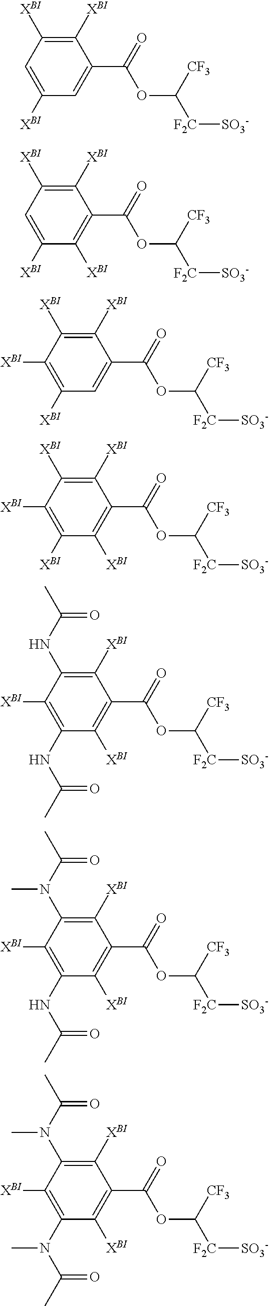

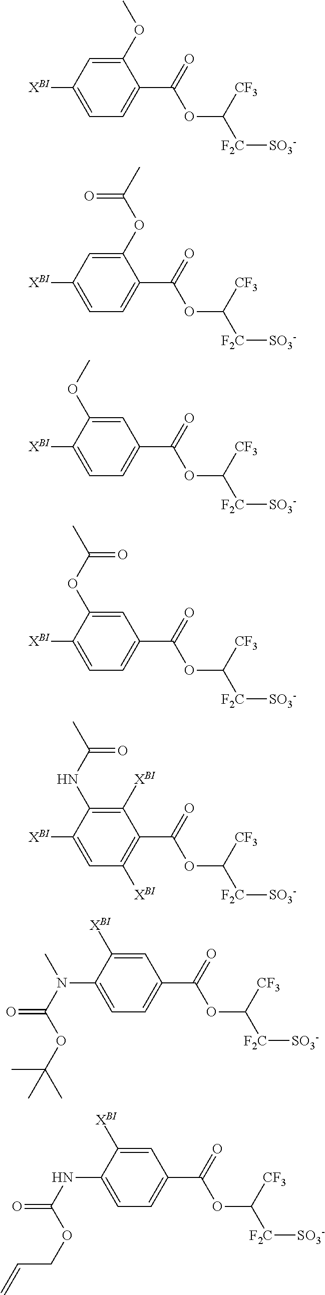

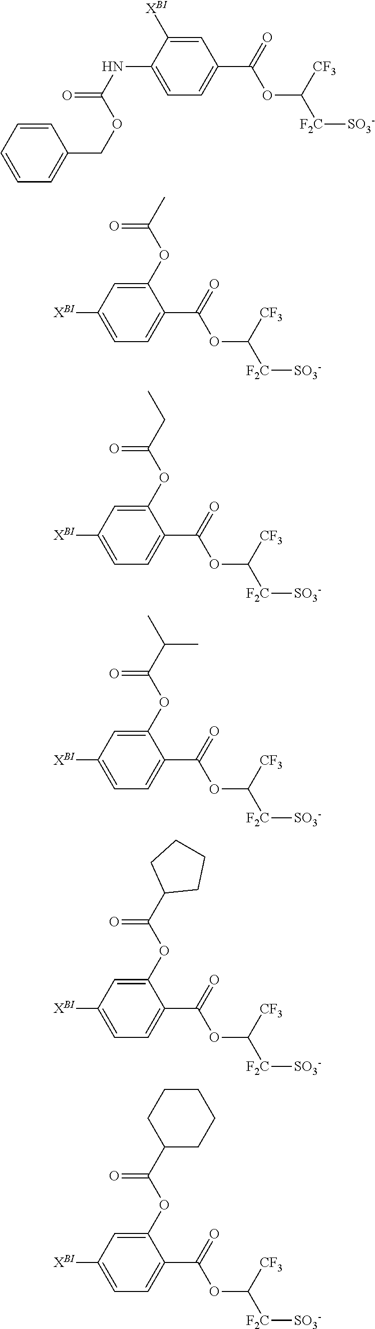

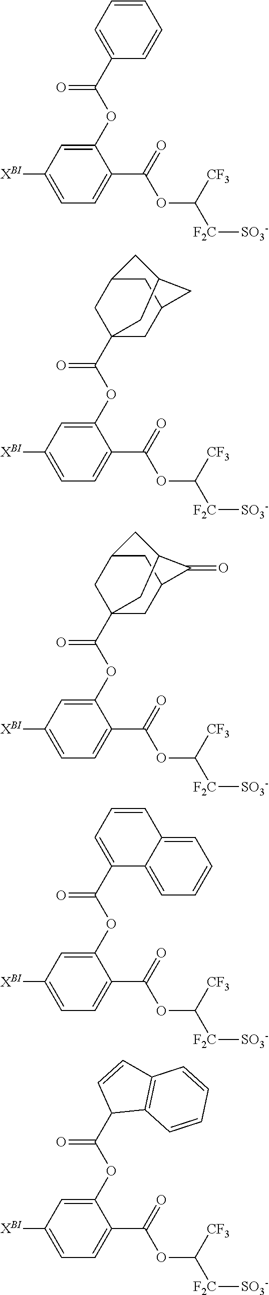

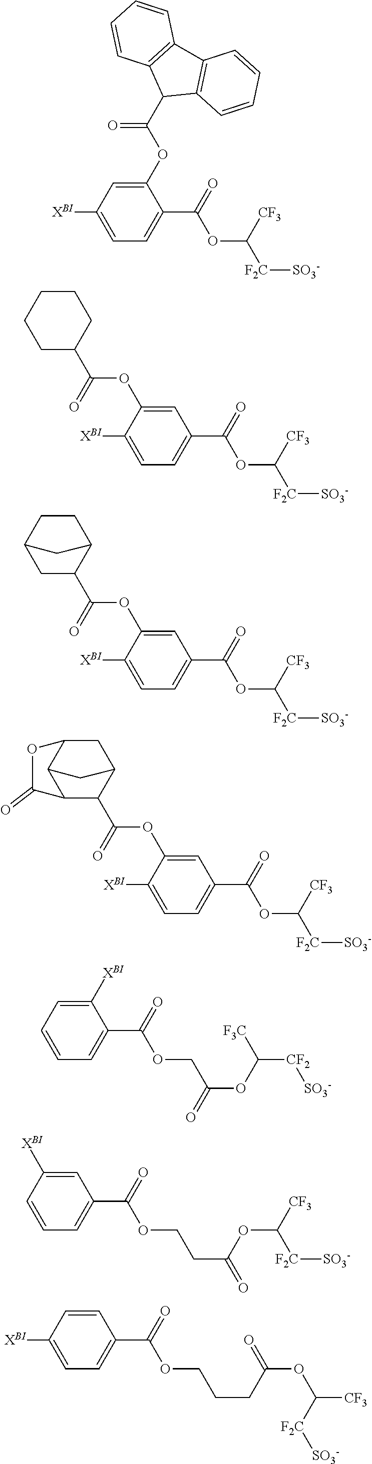

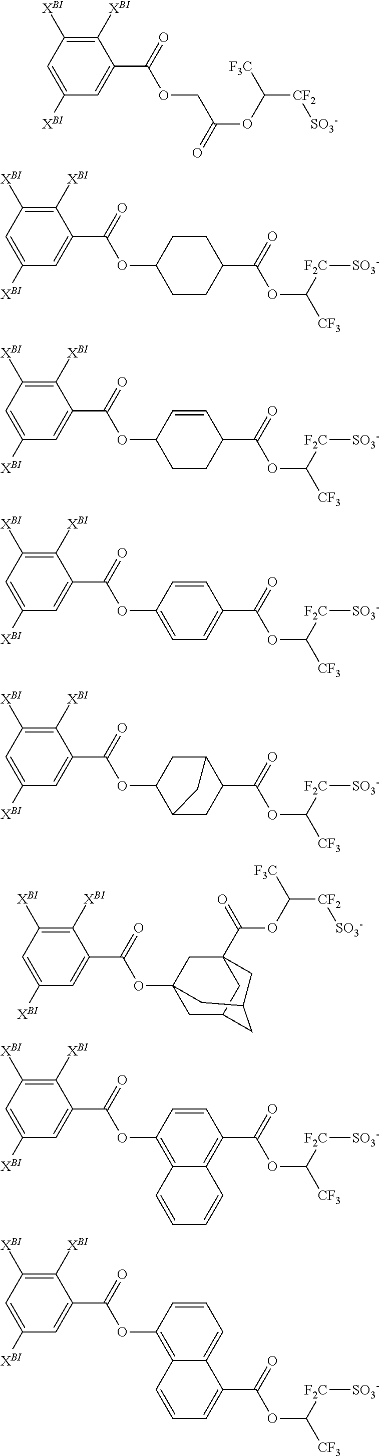

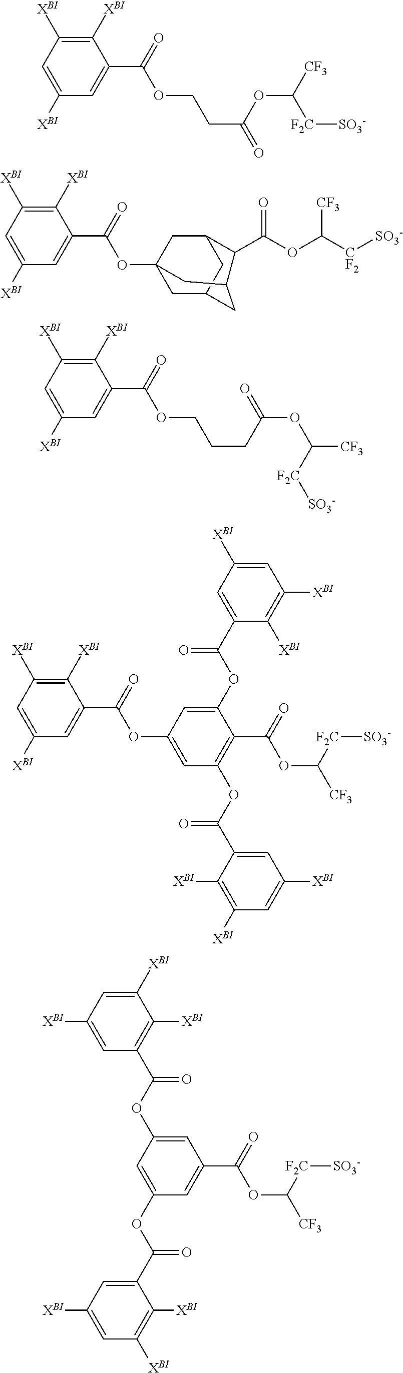

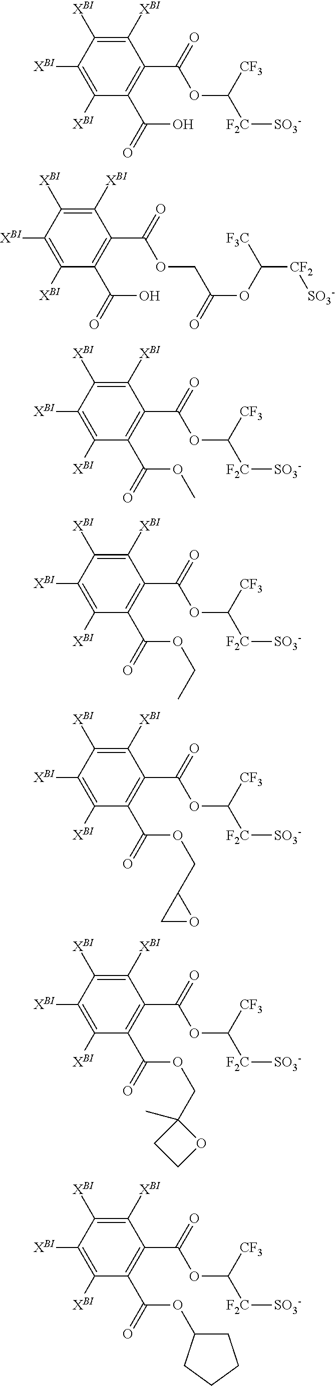

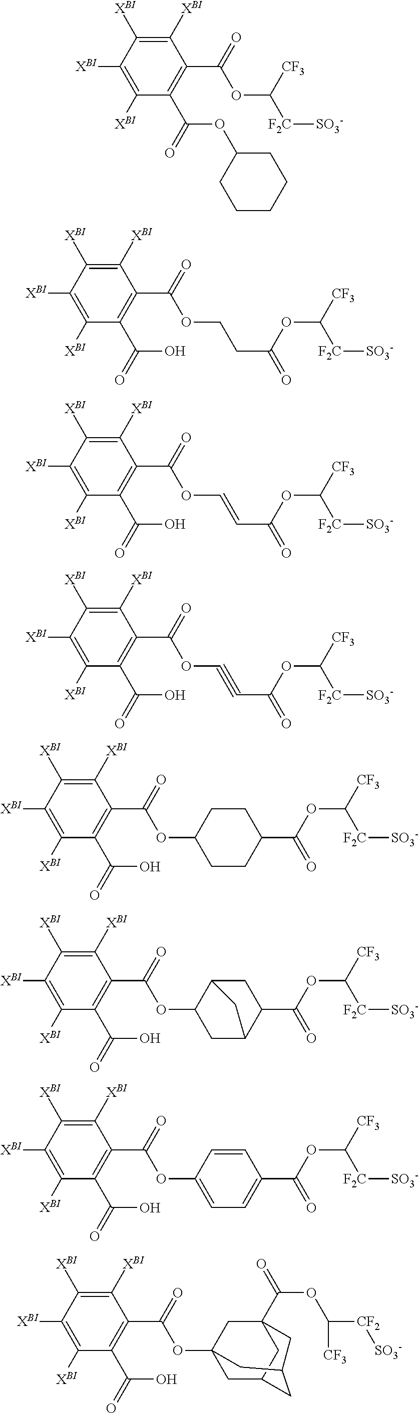

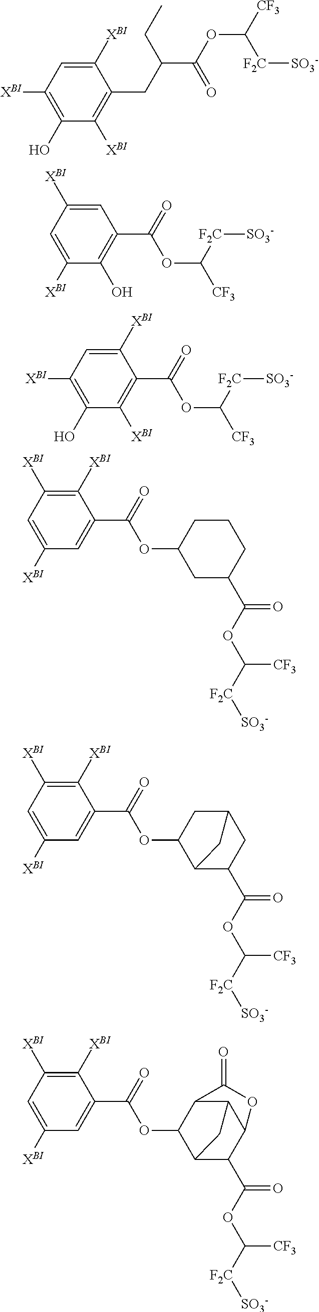

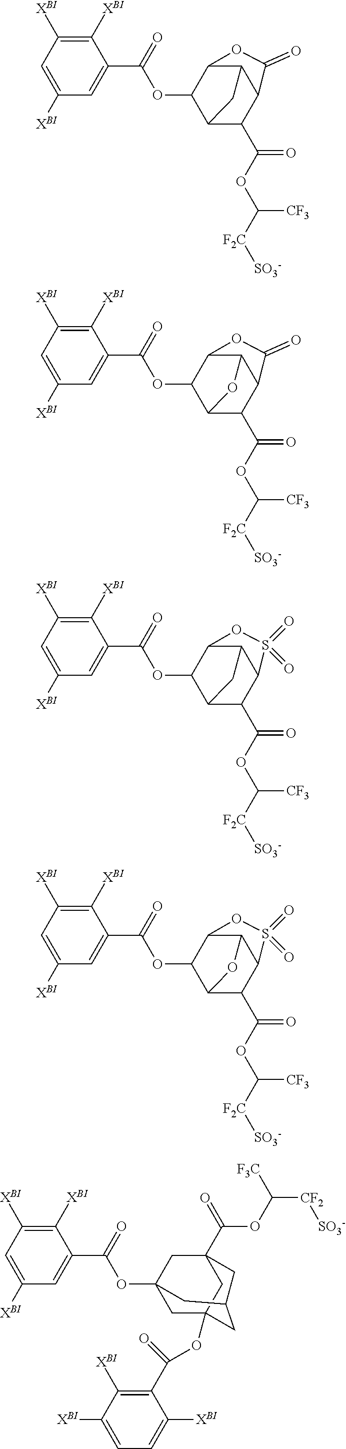

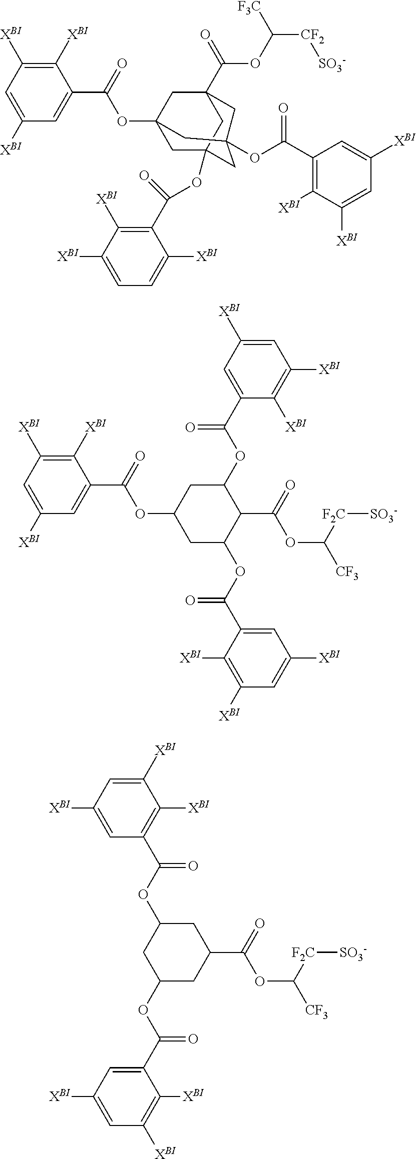

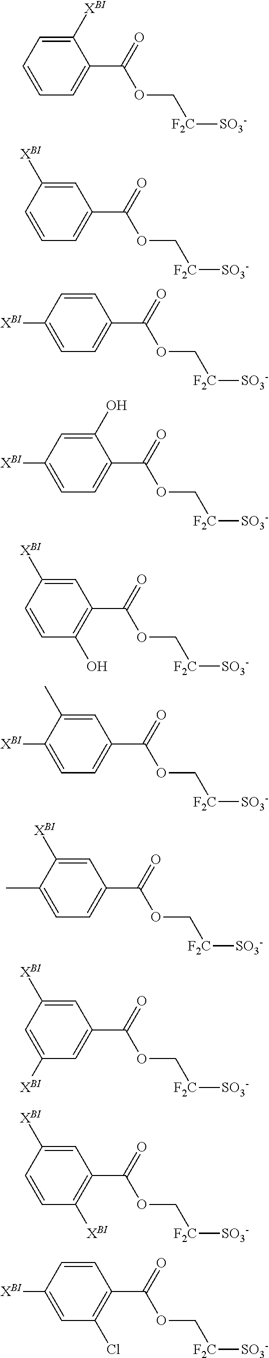

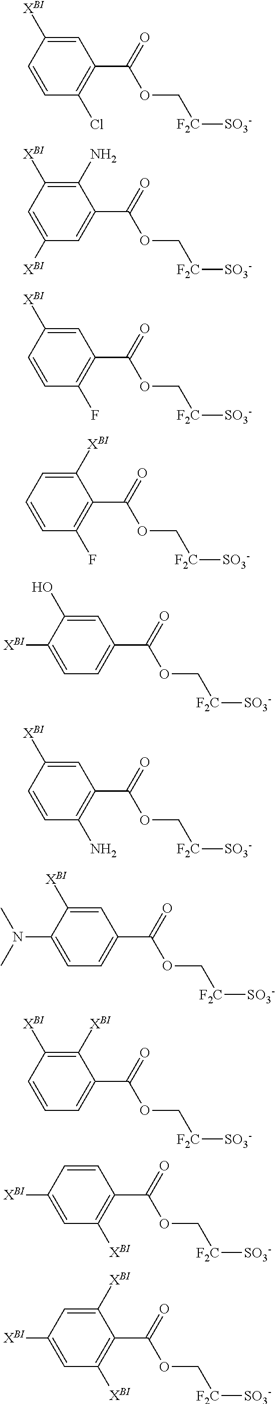

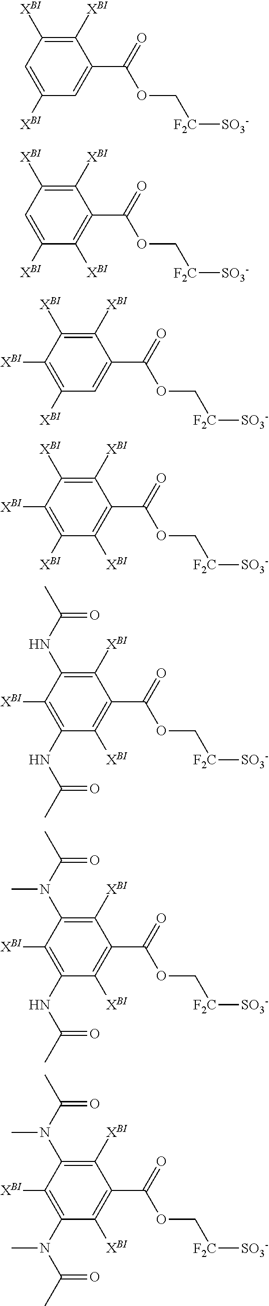

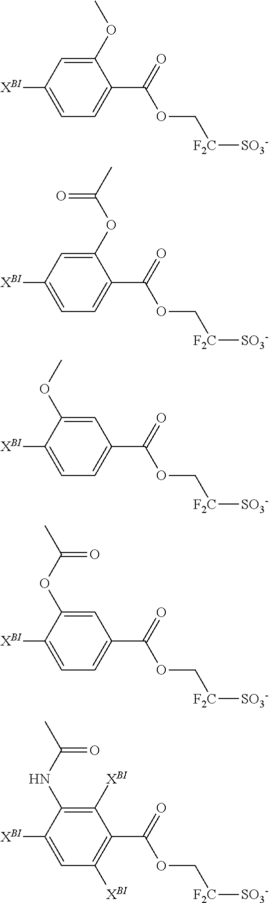

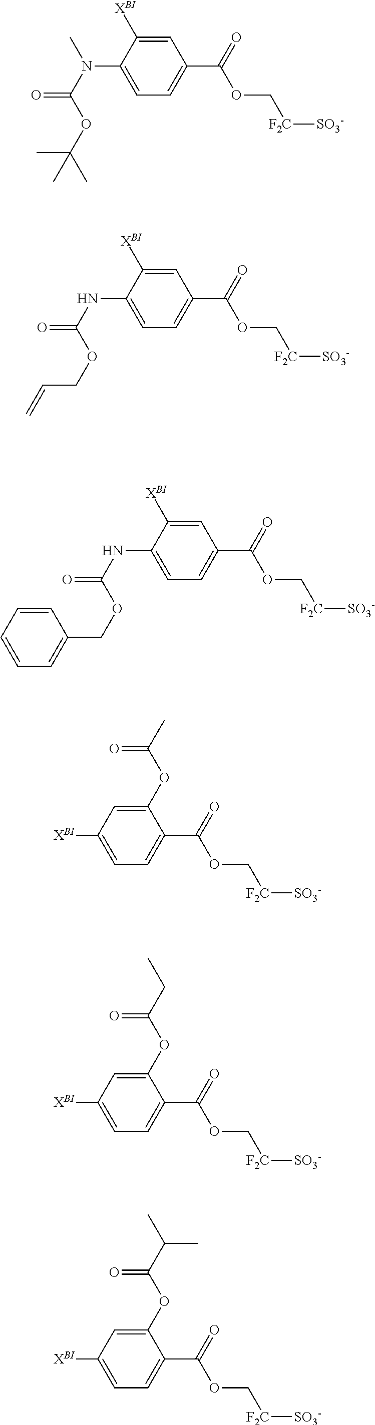

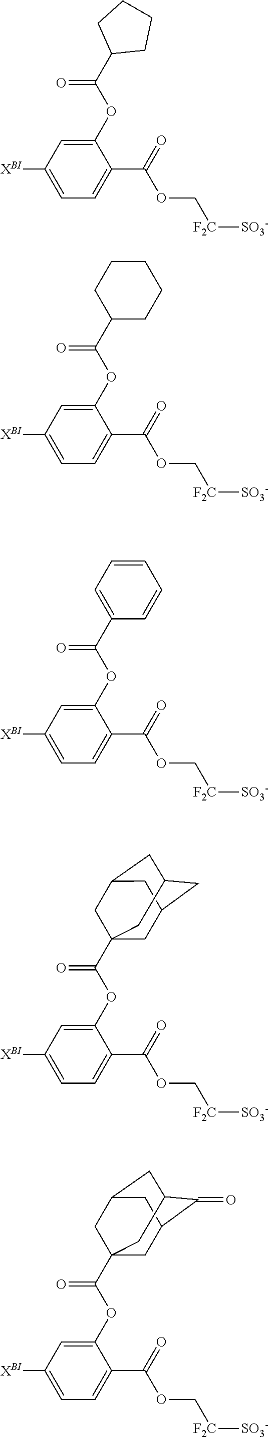

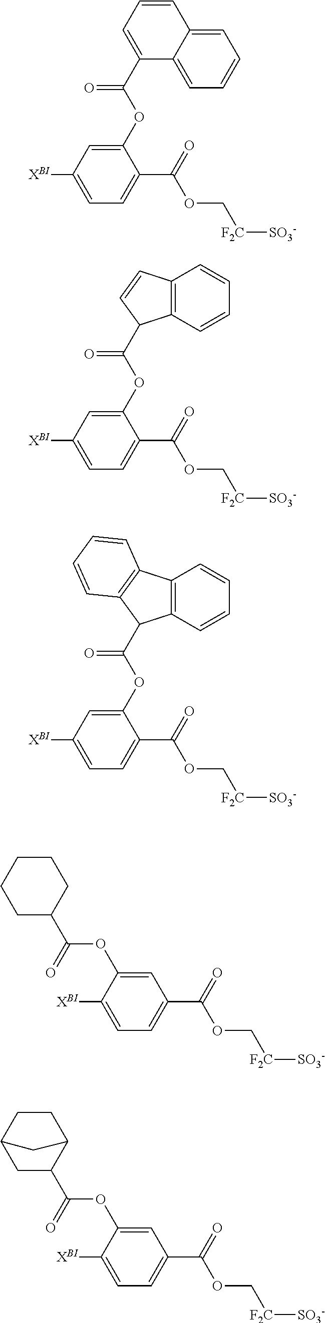

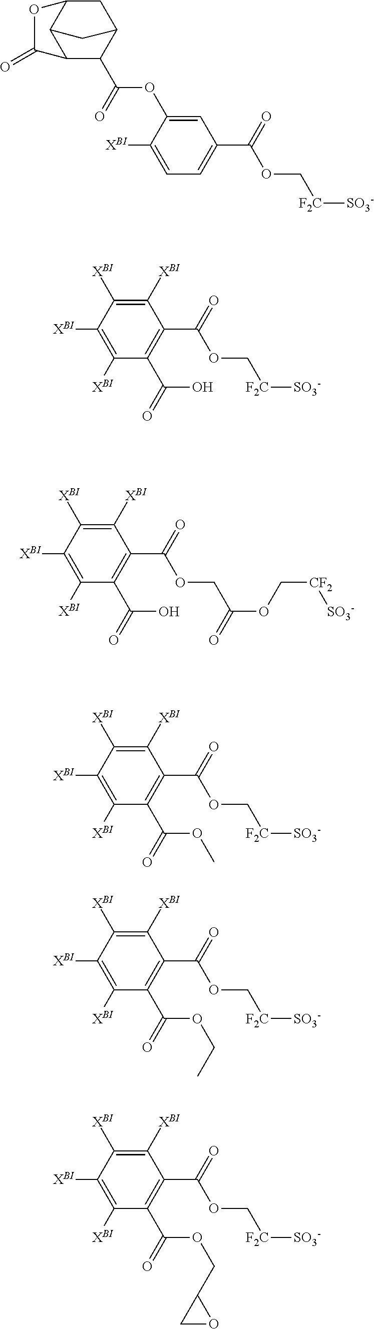

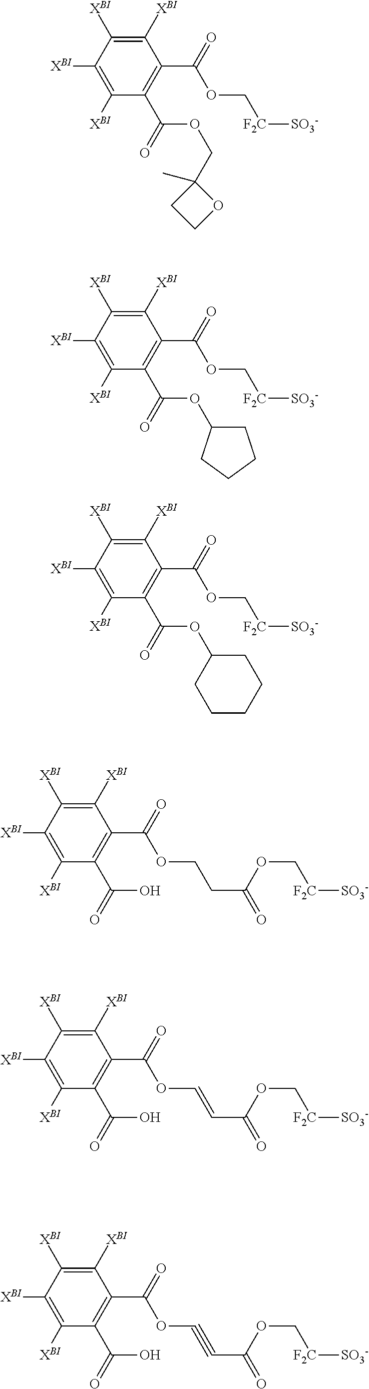

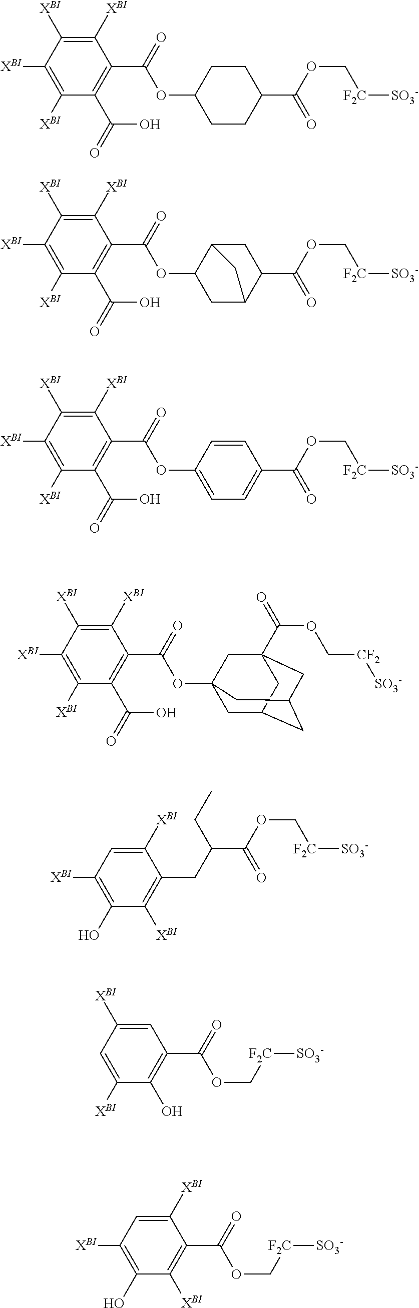

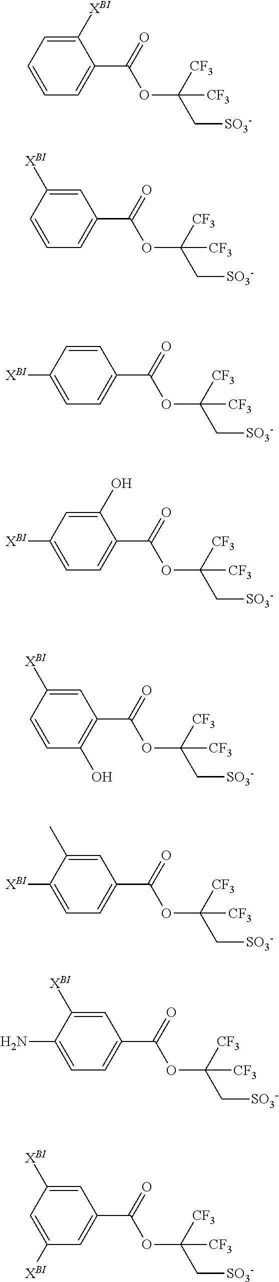

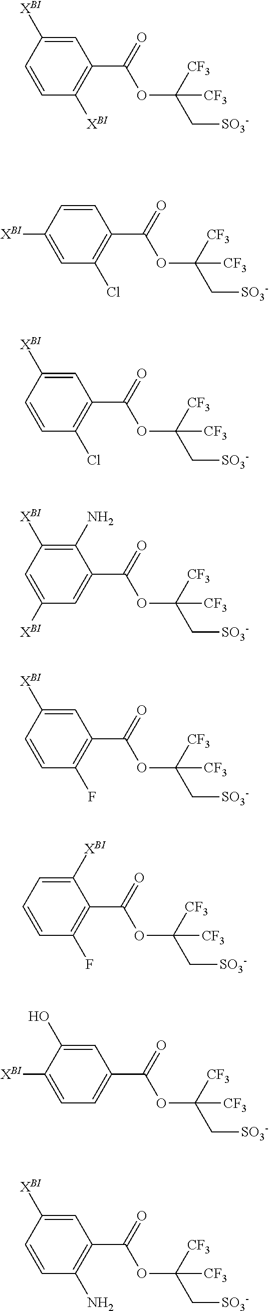

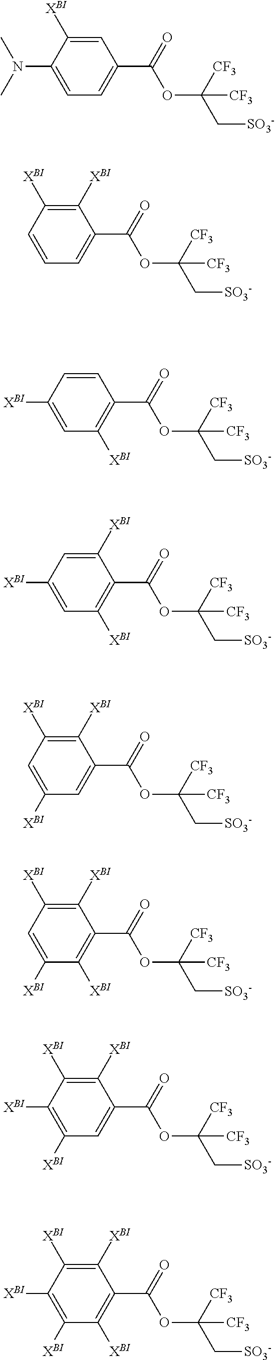

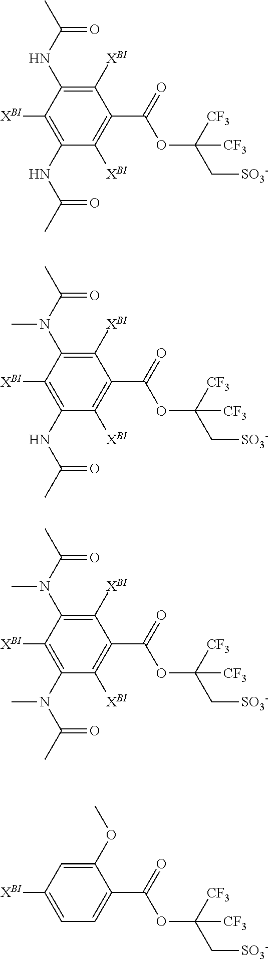

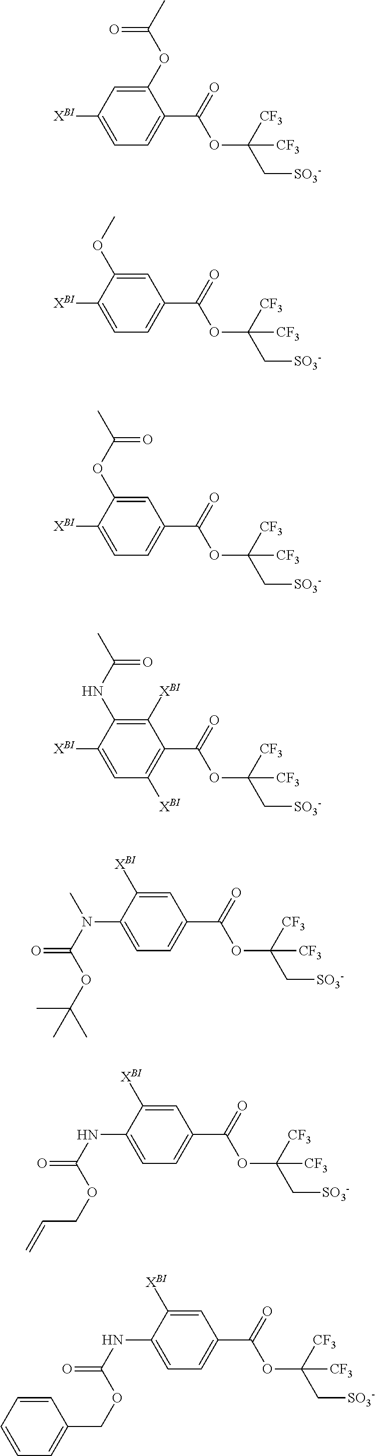

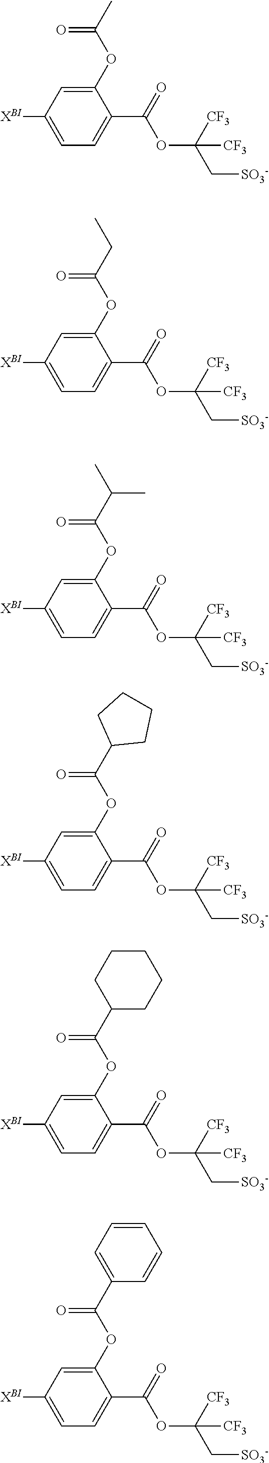

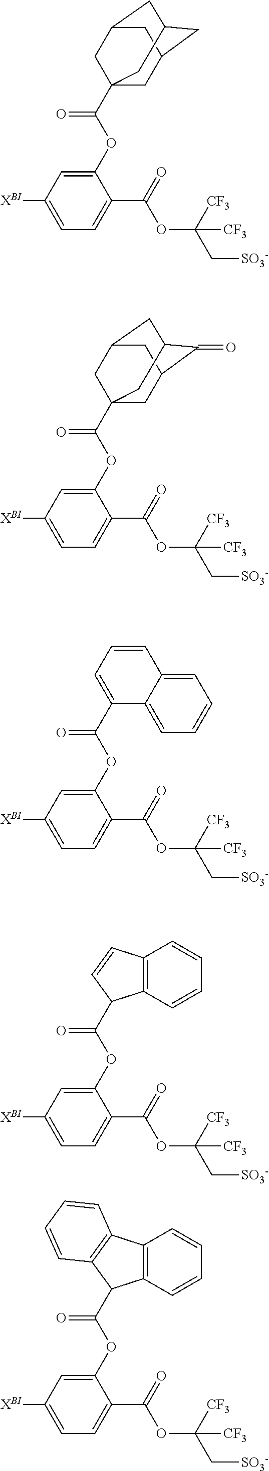

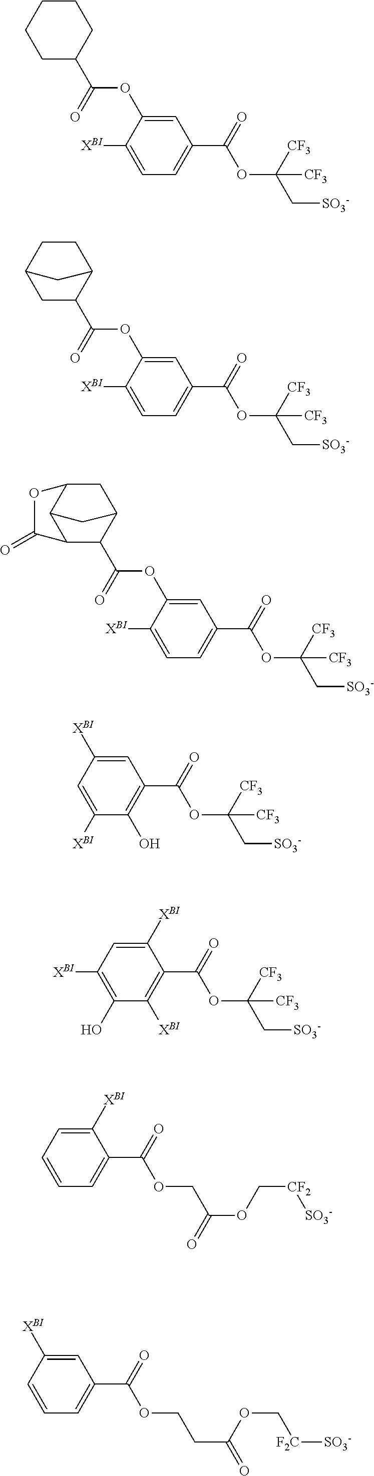

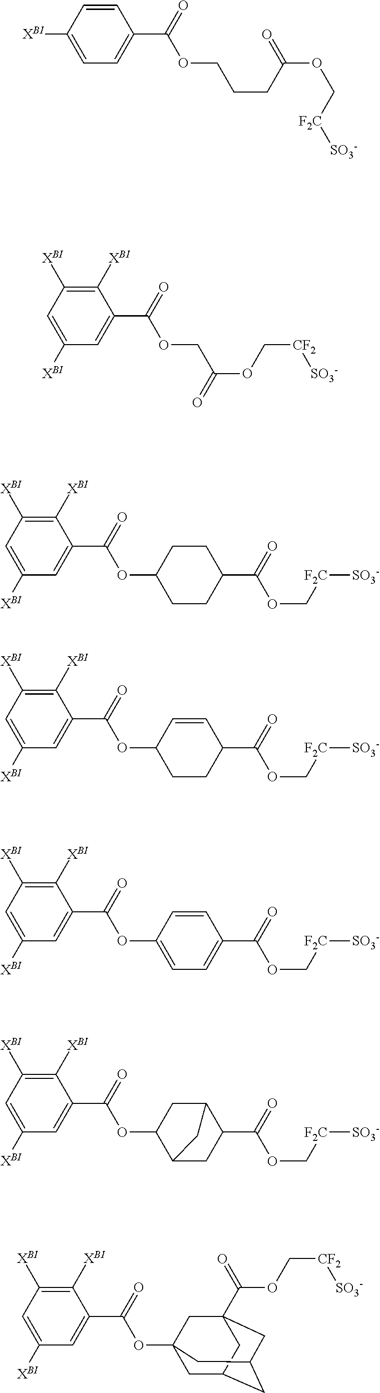

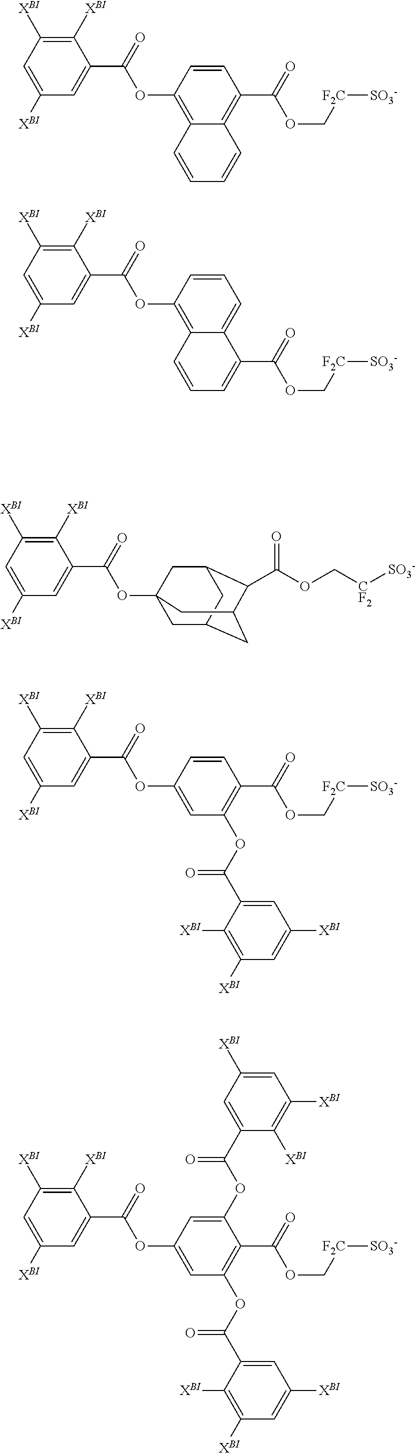

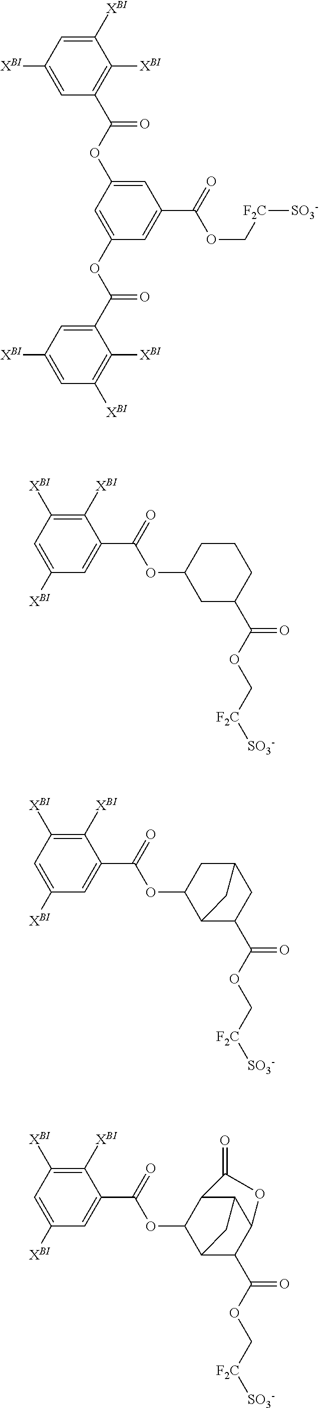

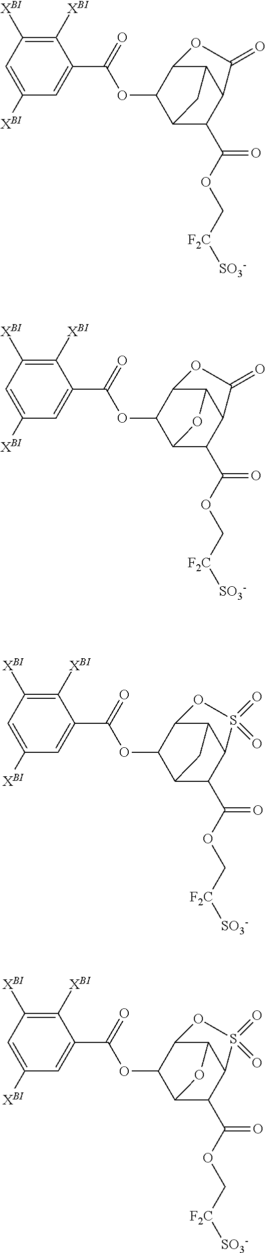

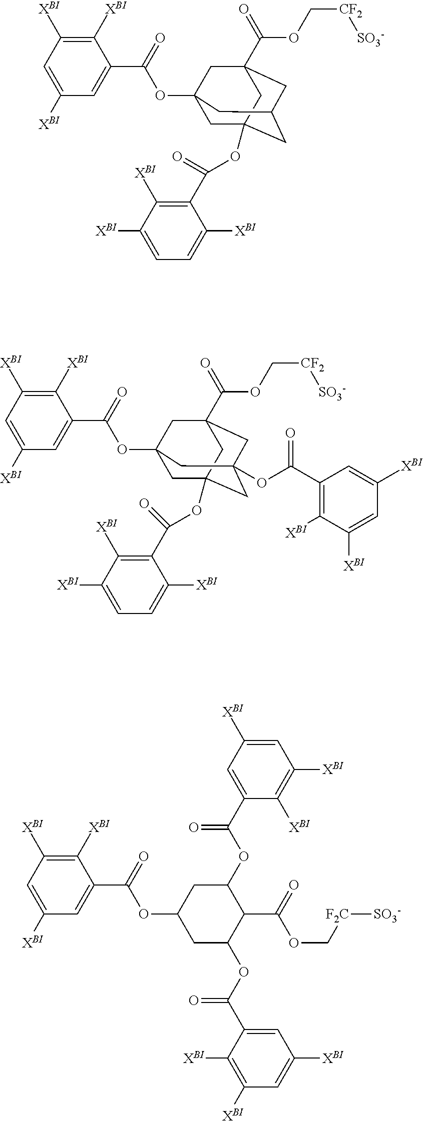

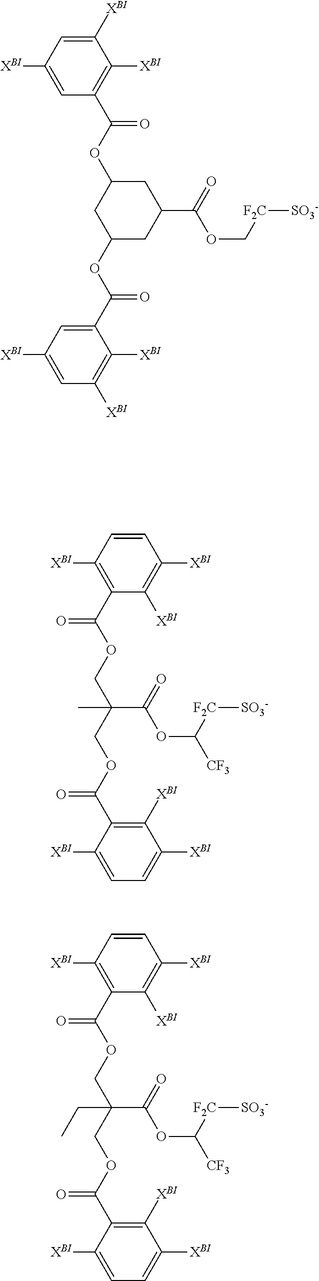

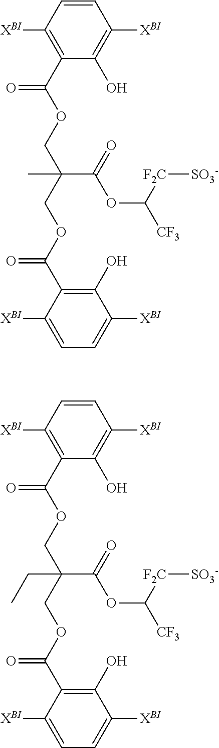

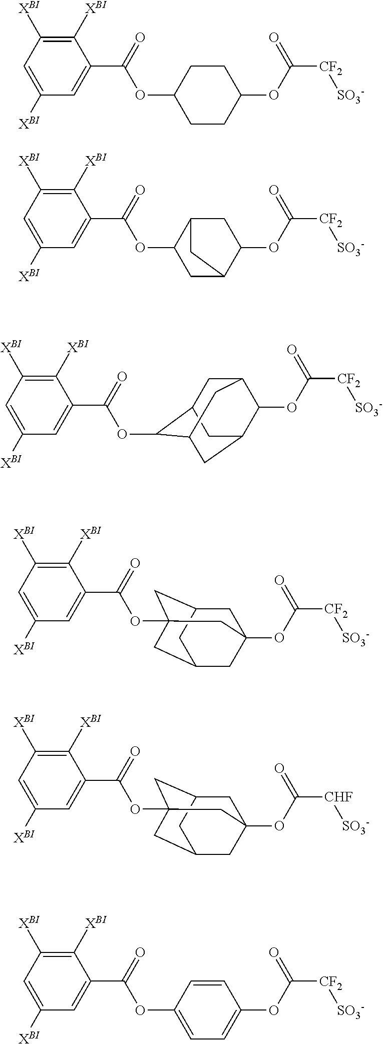

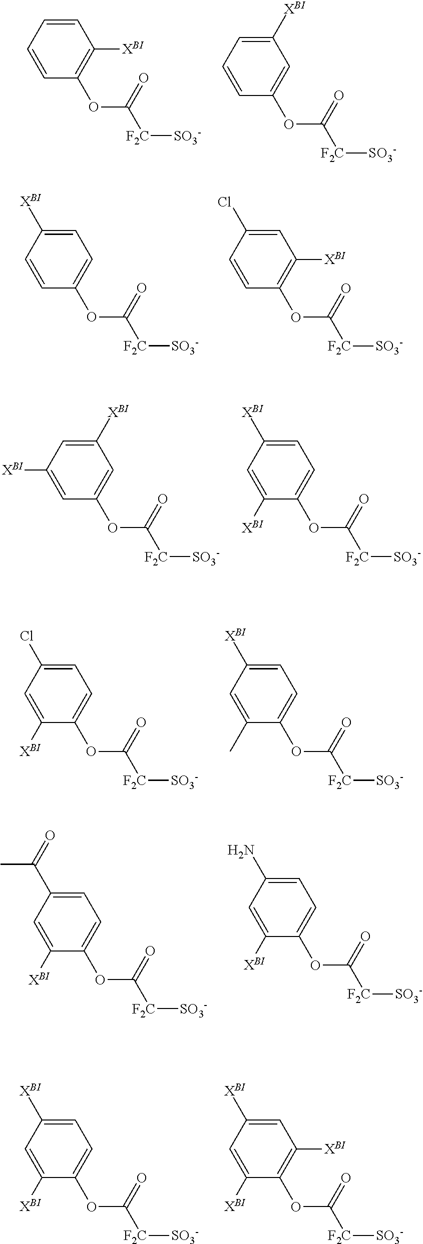

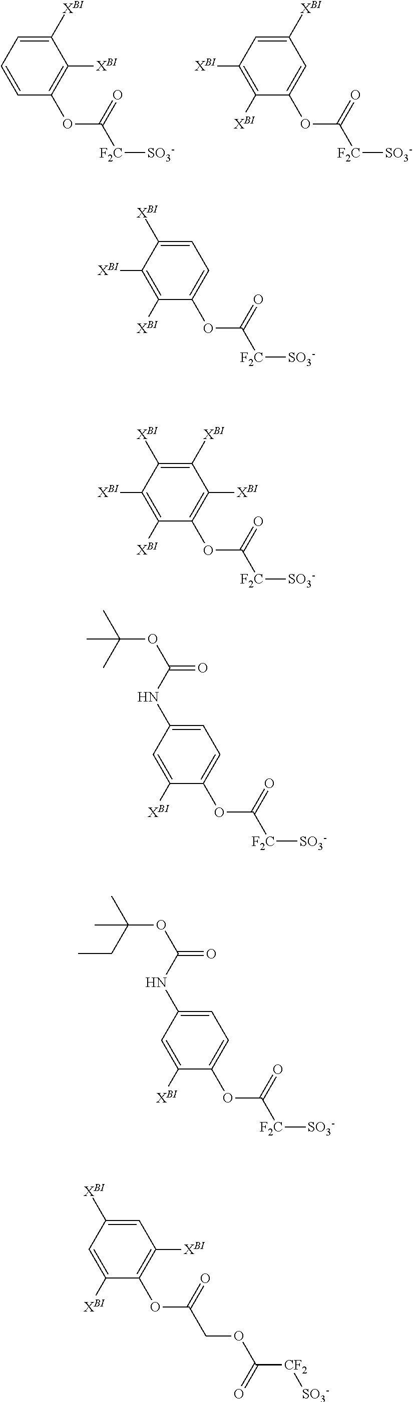

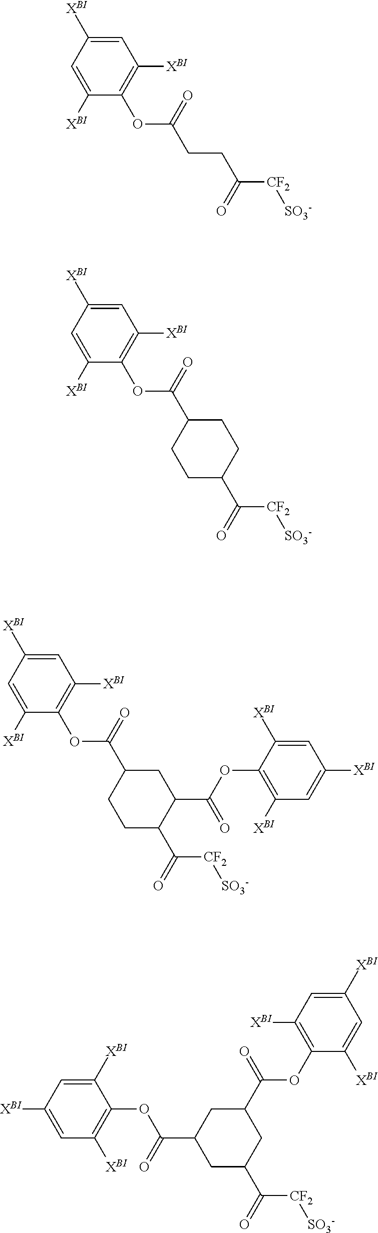

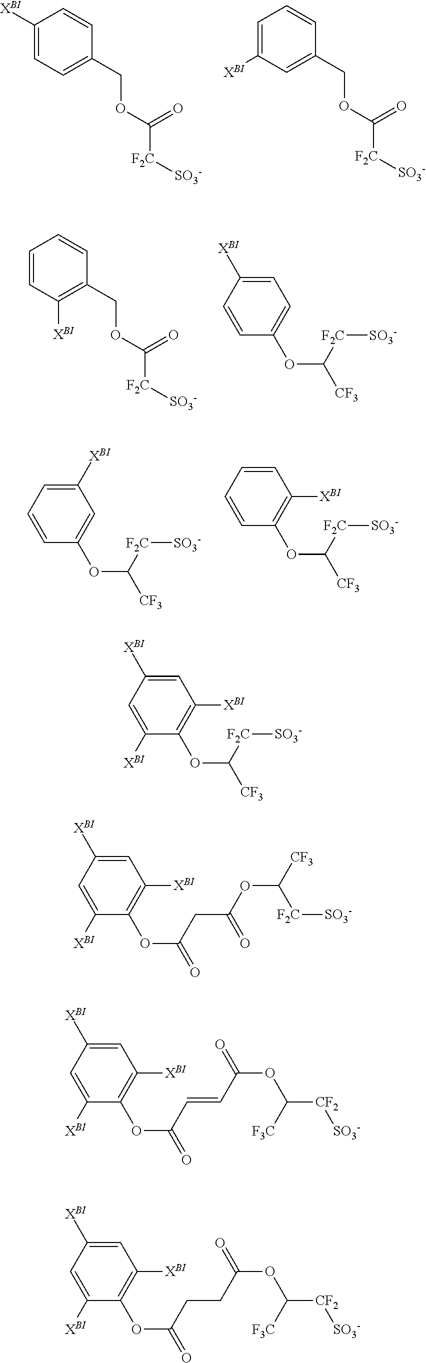

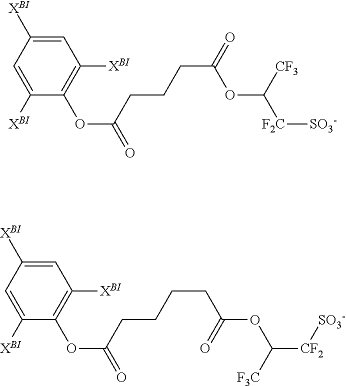

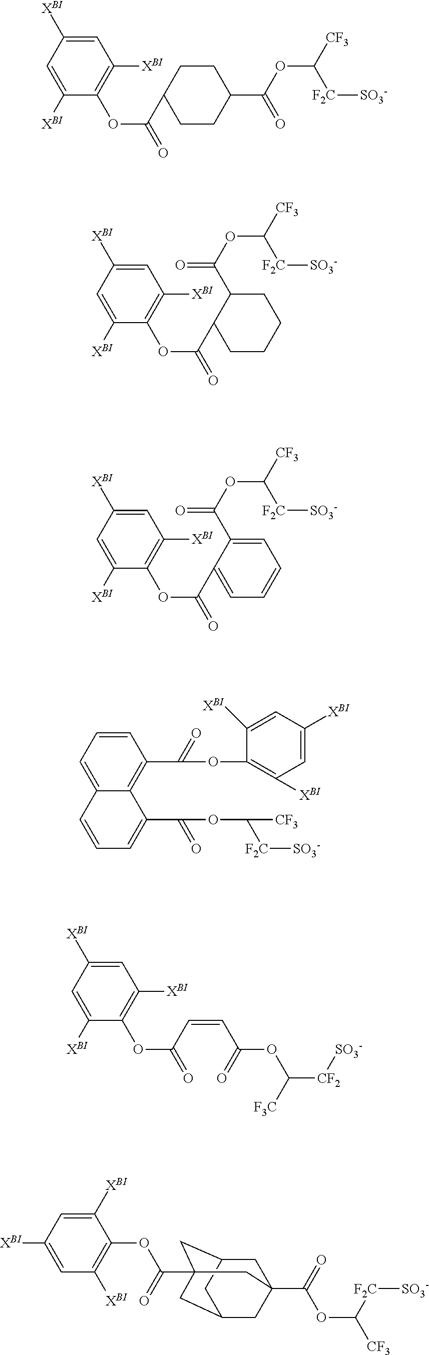

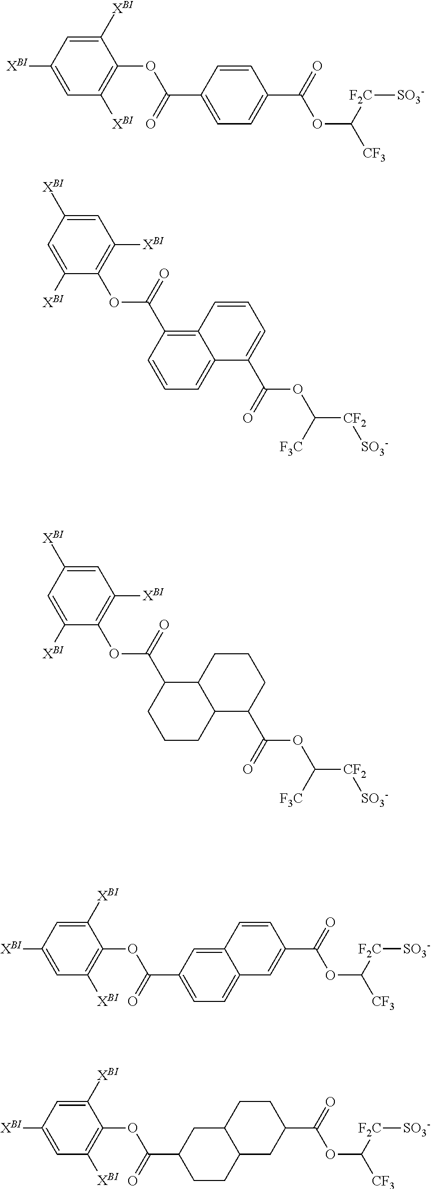

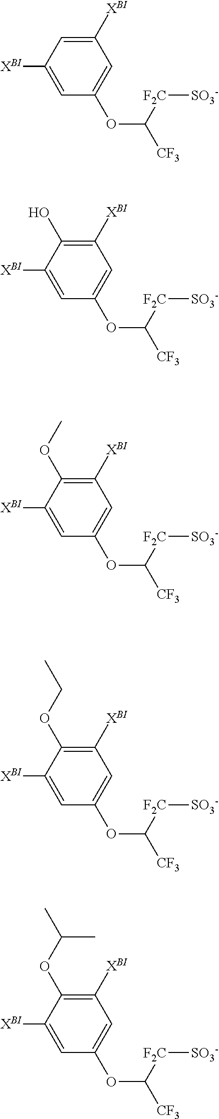

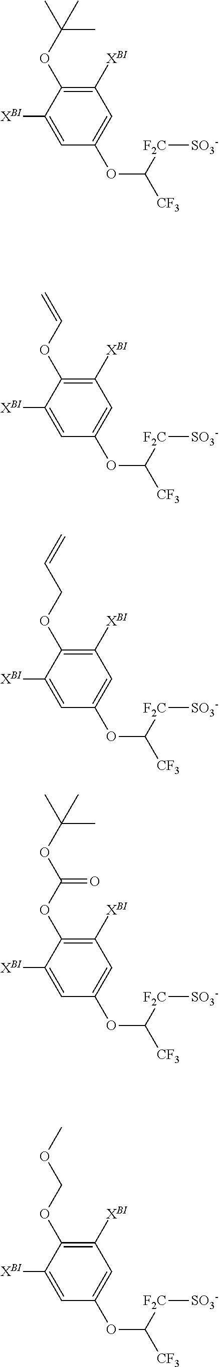

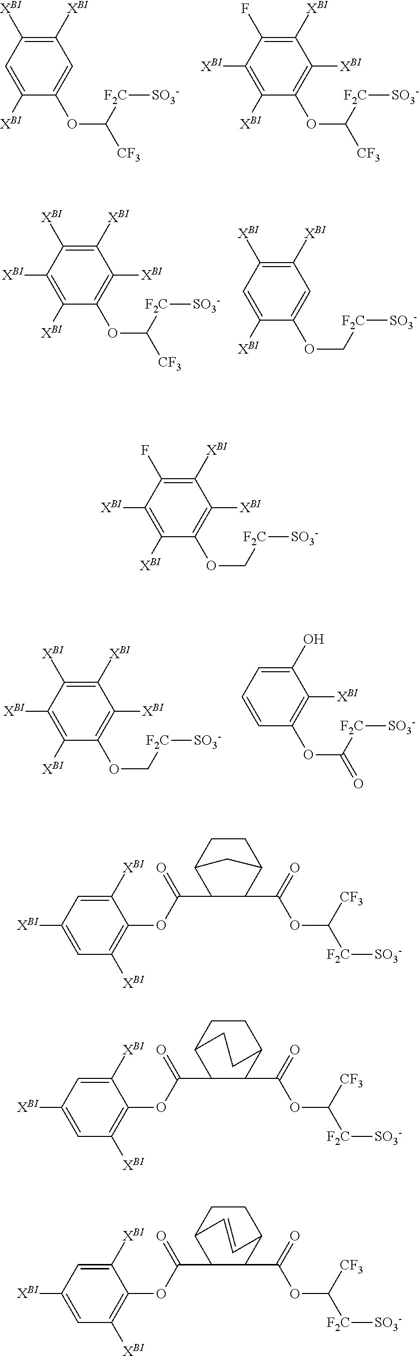

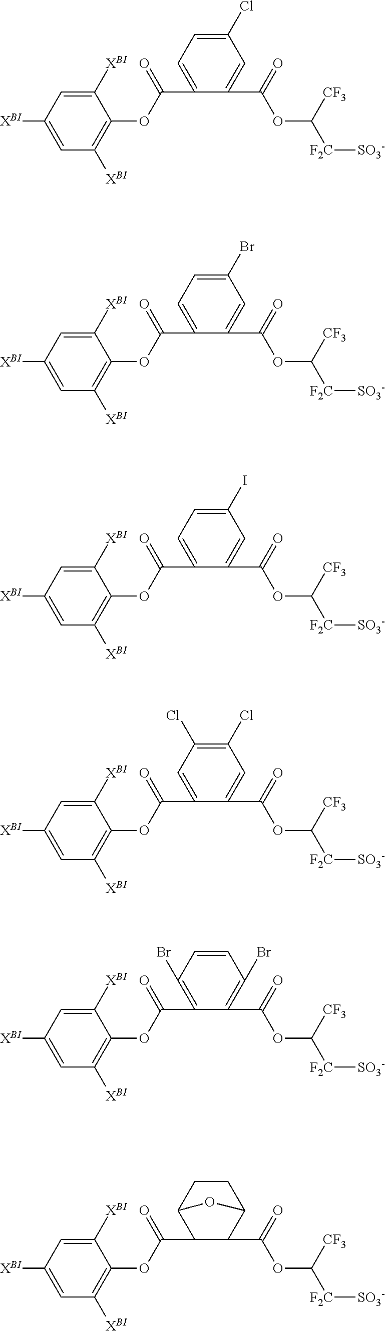

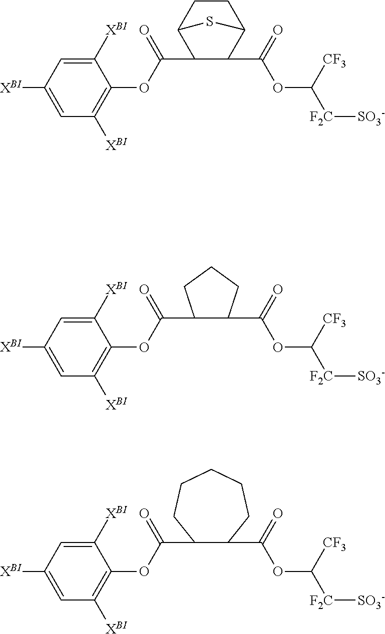

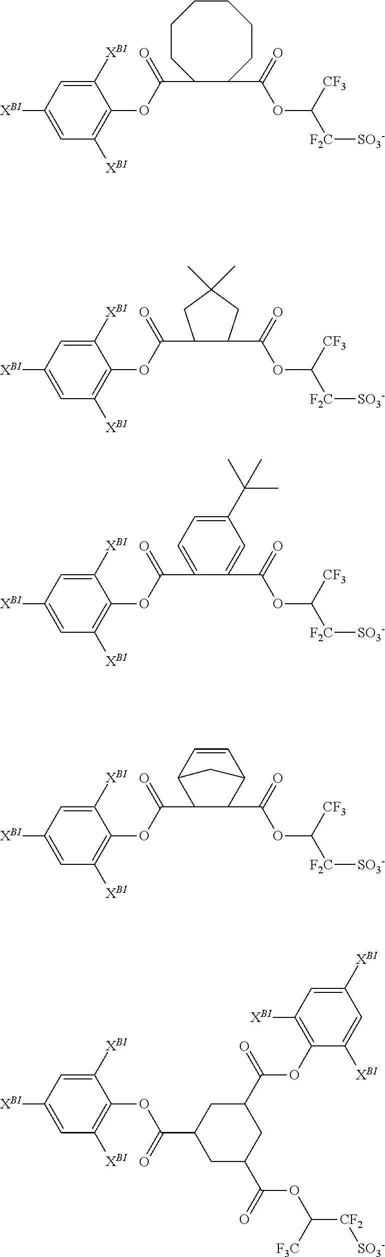

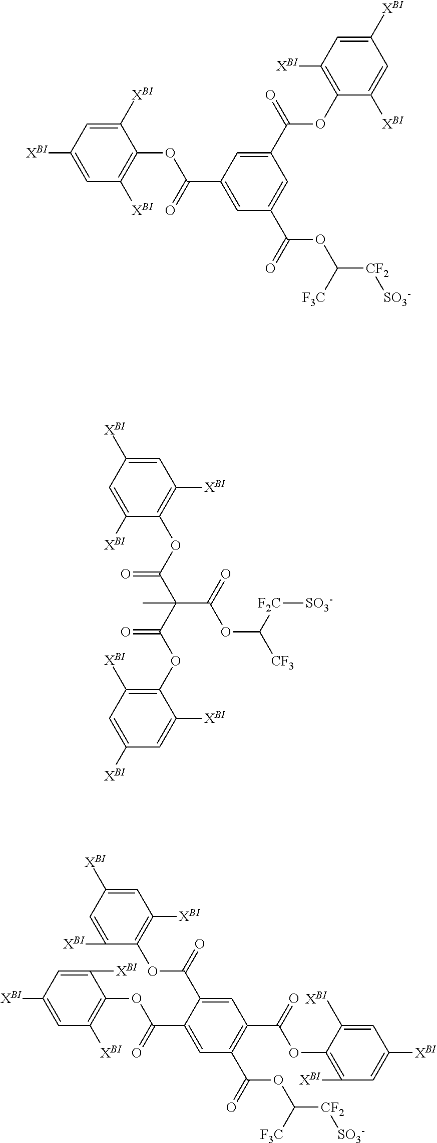

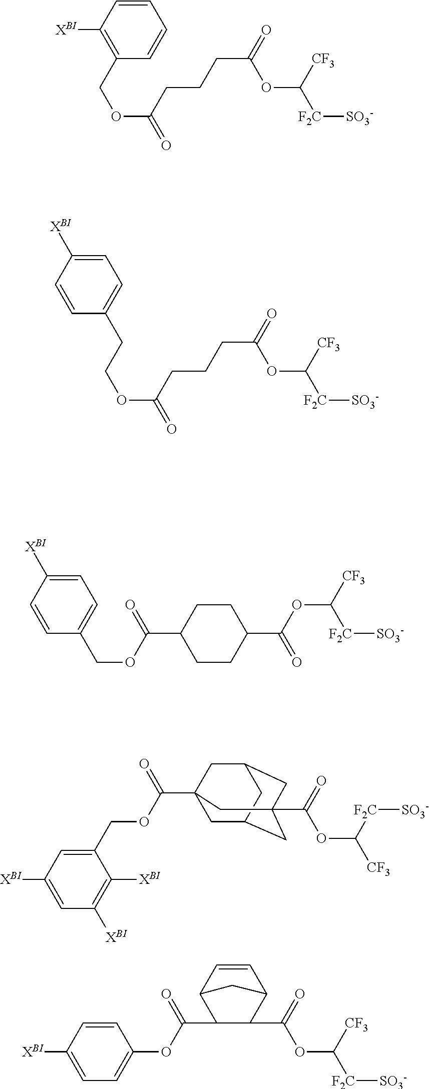

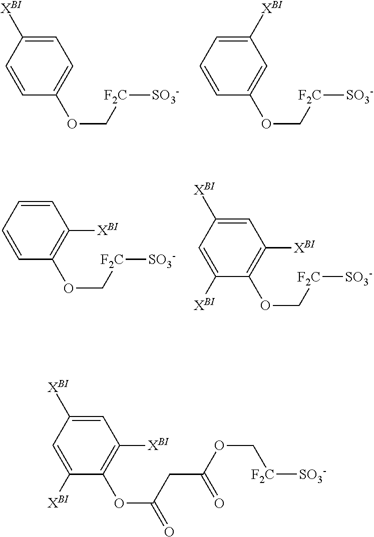

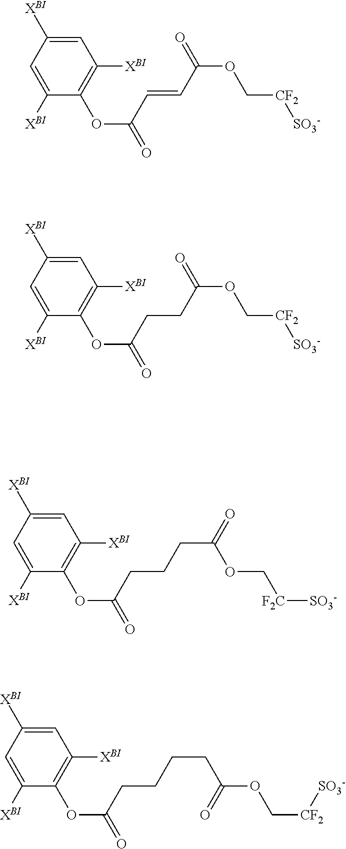

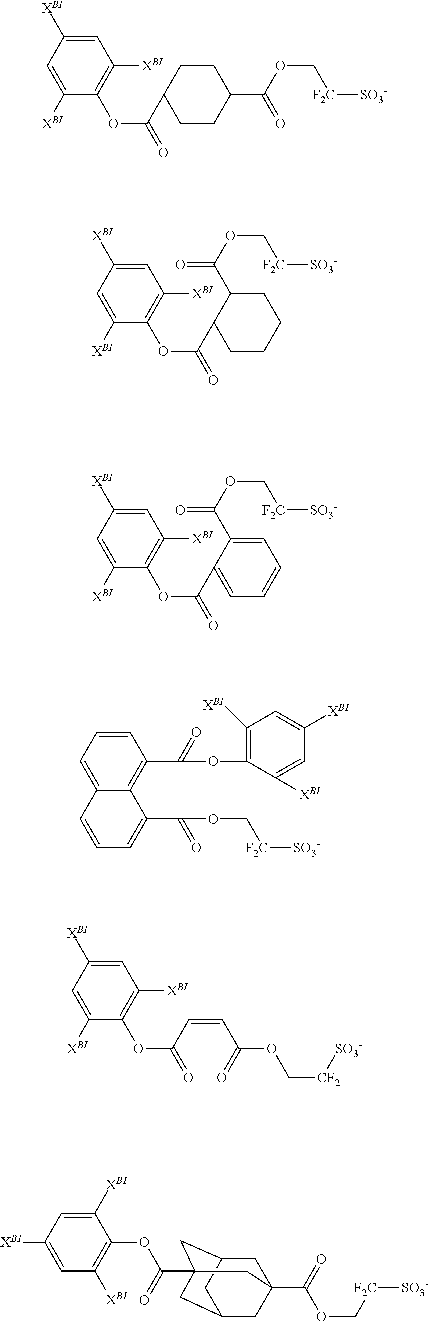

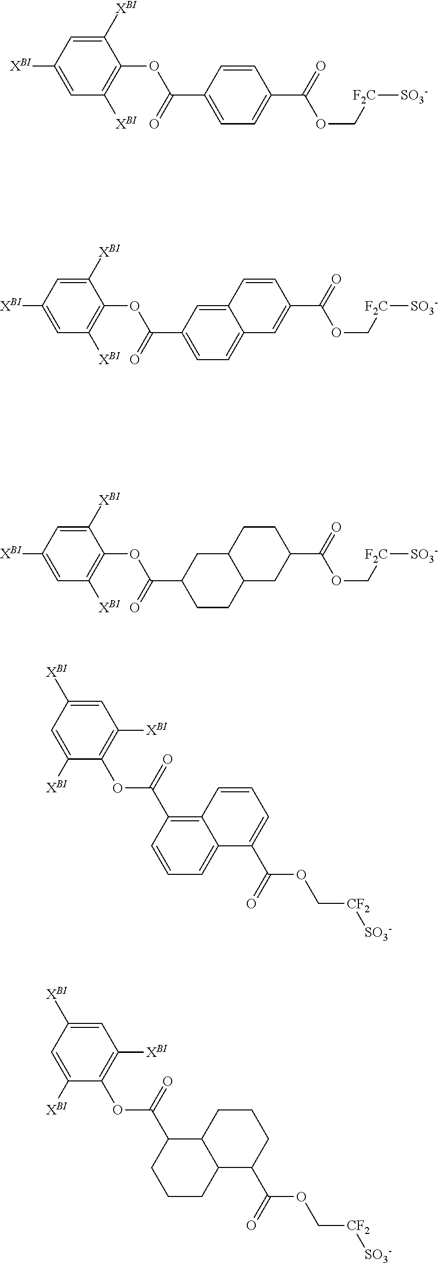

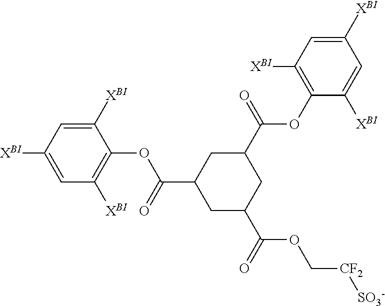

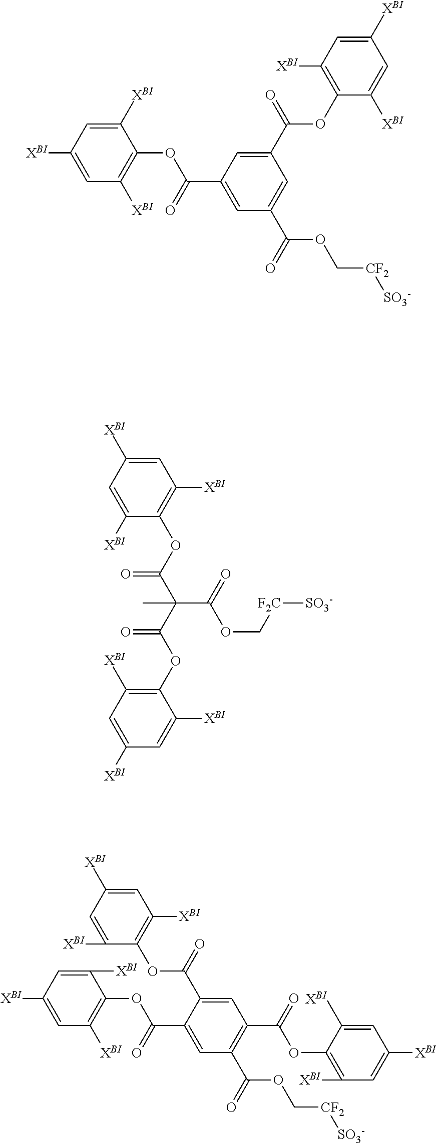

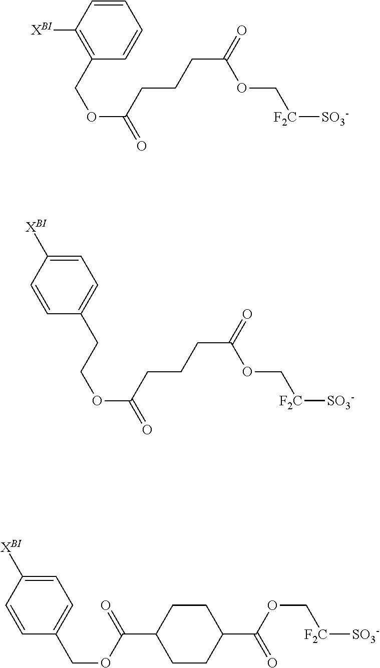

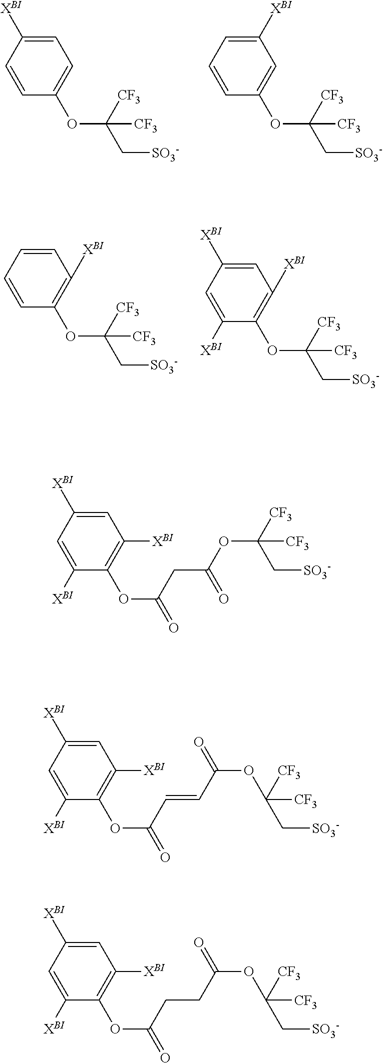

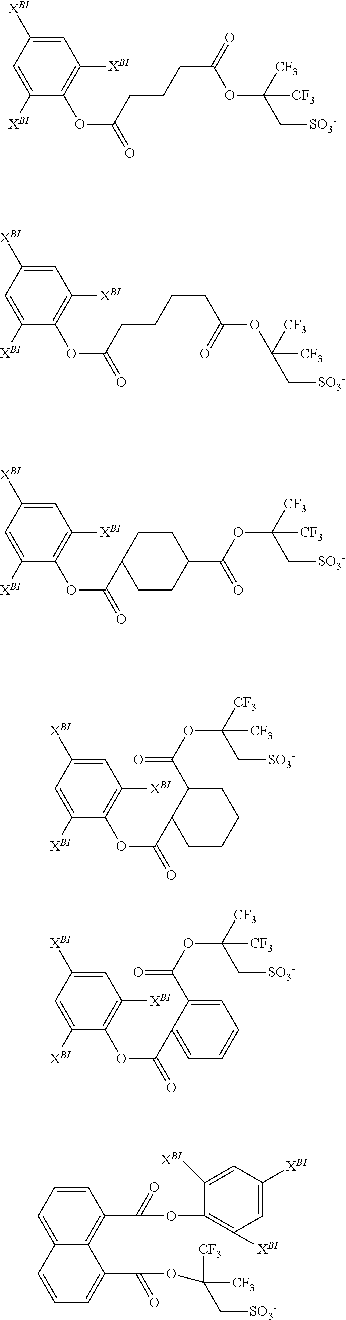

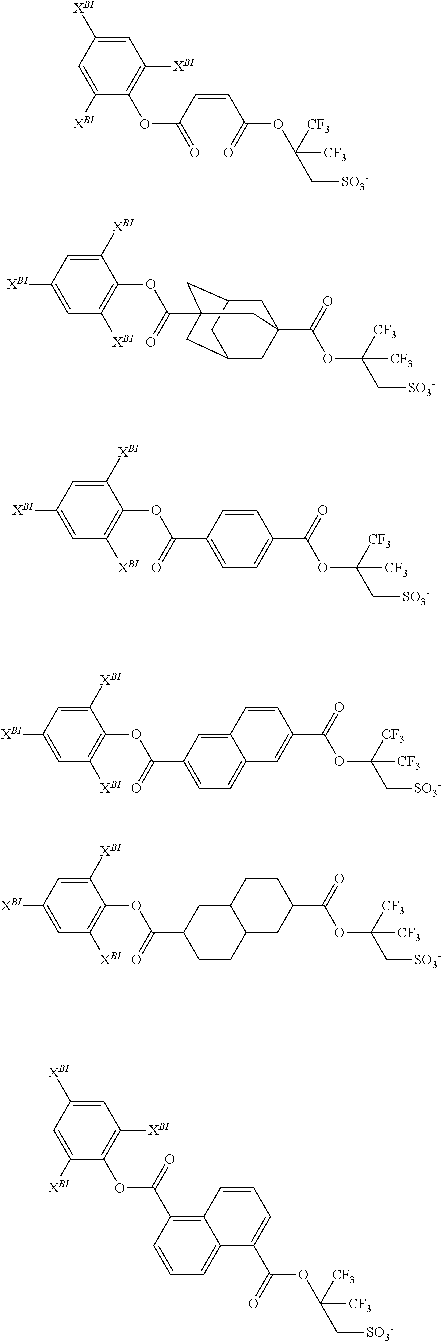

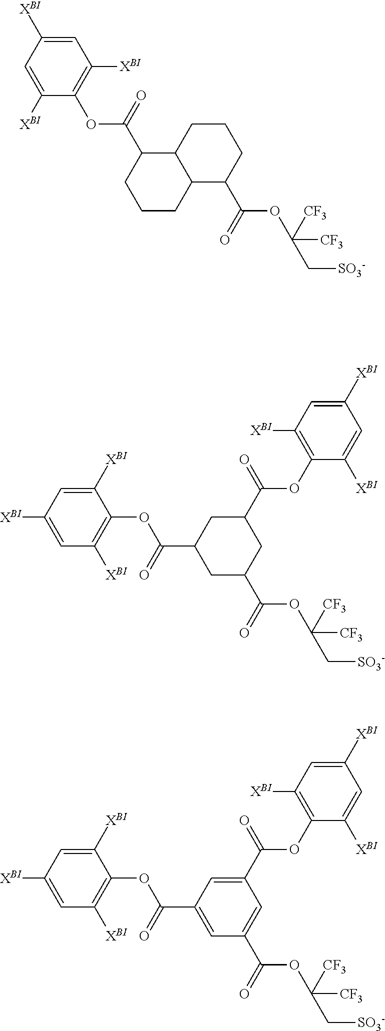

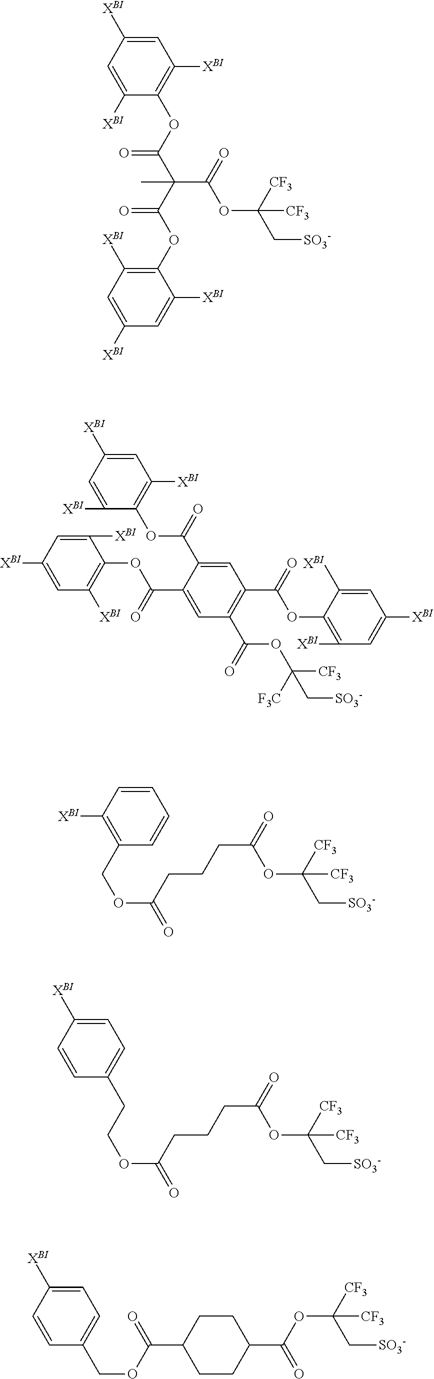

Other examples of the anion of M⁻ include those described in paragraphs [0076] and [0106] of WO 2023/157455, paragraph [0111] of WO 2024/24801, paragraphs [0253] to [0256] of WO 2024/43121, paragraphs [0044] and [0045] of WO 2024/57751, paragraphs [0205] to [0220] of WO 2024/122423, paragraphs [0170] to [0178] of JP-A 2023-123183, paragraphs [0026] to [0028] of JP-A 2024-62406, paragraphs [0022] to [0025] of JP-A 2024-62407, paragraphs [0026] to [0028] of JP-A 2024-62408, paragraphs [0028] to [0030] of JP-A 2024-68156, paragraphs [0026] to [0028] of JP-A 2024-68157, paragraphs [0028] to [0030] of JP-A 2024-68158, paragraphs [0028] to [0030] of JP-A 2024-68159, paragraphs [0031] to [0033] of JP-A 2024-72280, paragraphs [0023] to [0025] of JP-A 2024-72281, paragraphs [0026] to [0029] of JP-A 2024-77618, paragraphs [0020] and [0021] of JP-A 2024-77619, paragraphs [0140] to [0143] of JP-A 2024-80672, paragraphs [0023] to [0025] of JP-A 2024-83303, paragraphs [0028] to [0031] of JP-A 2024-83304, paragraphs [0030] to [0033] of JP-A 2024-99500, paragraphs [0028] to [0030] of JP-A 2024-99502, paragraphs [0030] to [0032] of JP-A 2024-101557, paragraphs [0025] to [0027]of JP-A 2024-102842, paragraphs [0033] to [0035] of JP-A 2024-102843, paragraphs [0021] and [0022] of JP-A 2024-127832, paragraphs [0169] to [0172] of JP-A 2024-144354, paragraphs [0178] to [0181] of JP-A 2024-144356, paragraphs [0040] to [0143] of JP-A 2024-160436, paragraphs [0030] and [0031] of JP-A 2025-705, paragraphs [0157] and [0158] of JP 7247732, paragraphs [0227] to [0238] of JP 7446352, paragraphs [0253] to [0256] of JP 7466597, and paragraphs [0309] to [0312] of JP 7466782.

Other examples of the non-nucleophilic counter ion further include fluorine-free aromatic sulfonic acid anions and alkanesulfonic acid anions described in paragraphs [0186] to [0188] of JP 7398551, paragraphs [0028] and [0029] of JP 7067271, paragraphs [0220] to [0225] of JP 7032549, paragraphs [0040] to [0045] of JP 6645464, paragraphs [0027] to [0029] of JP 6248882, paragraphs [0034] to [0037] of JP 6090585, paragraphs [0037] to [0060] of JP-A 2024-104830, paragraphs [0033] to [0093] of JP-A 2024-140135, paragraphs [0039] to [0066] of JP-A 2023-177038, and paragraphs [0100] to [0102] of JP-A 2006-276759. Examples thereof further include fluorine-containing aromatic sulfonic acid anions described in paragraphs [0029] to [0034] of JP 6648726, paragraphs [0264] to [0267] of JP-A 2024 077330, and paragraphs [0040] to [0071] of JP-A 2025 000201.

As the non-nucleophilic counter ion, an anion of a bissulfonic acid described in JP-A 2015-206932, an anion having a sulfonic acid on one side and having a sulfonamide or a sulfonimide different from the sulfonic acid on the other side, described in WO 2020/158366, and an anion having a sulfonic acid on one side and having a carboxylic acid on the other side, described in JP-A 2015-24989 can be further used.

In the formulas (c2) and (c3), d1 and d2 are each independently 0, 1, 2, or 3, but preferably 1.

In the formula (c4), e1 is 0 or 1. e2 is 0, 1, 2, 3, or 4. e3 is 0, 1, 2, 3, or 4. Provided that 0≤e2+e3≤4 when e1 is 0, and 0≤e2+e3≤6 when e1 is 1.

In the formulas (c2), (c3) and (c4), L¹ is a single bond, an ether bond, an ester bond, a carbonyl group, a sulfonate bond, a sulfonamide bond, a carbonate bond, or a carbamate bond. Among these, an ether bond, an ester bond, and a carbonyl group are preferable, and an ester bond and a carbonyl group are more preferable from a viewpoint of synthesis.

In the formula (c2), Rf¹ and Rf² are each independently a fluorine atom or a C₁-C₆ fluorinated saturated hydrocarbyl group. Among these, Rf¹ and Rf² are each preferably a fluorine atom in order to increase acid strength of a generated acid. Rf³ and Rf⁴ are each independently a hydrogen atom, a fluorine atom, or a C₁-C₆ fluorinated saturated hydrocarbyl group. Among these, at least one of Rf³ and Rf⁴ is preferably a trifluoromethyl group in order to improve solvent solubility.

In the formula (c3), Rf⁵ and Rf⁶ are each independently a hydrogen atom, a fluorine atom, or a C₁-C₆ fluorinated saturated hydrocarbyl group. Provided that not all Rf⁵s and Rf⁶s are simultaneously hydrogen atoms. Among these, at least one of Rf⁵ and Rf⁶ is preferably a trifluoromethyl group in order to improve solvent solubility.

In the formula (c4), Rf⁷ is a fluorine atom, a C₁-C₆ fluorinated alkyl group, a C₁-C₆ fluorinated alkoxy group, or a C₁-C₆ fluorinated alkylthio group. Rf⁷ is preferably a fluorine atom, a trifluoromethyl group, a difluoromethyl group, a trifluoromethoxy group, a difluoromethoxy group, a trifluoromethylthio group, or a difluoromethylthio group, and more preferably a fluorine atom, a trifluoromethyl group, or a trifluoromethoxy group. When e2 is 2, 3, or 4, Rf⁷s may be the same as or different from each other.

In the formula (c4), R⁴³ is a halogen atom other than a fluorine atom, or a C₁-C₂₀ hydrocarbyl group which may contain a heteroatom. The hydrocarbyl group may be saturated or unsaturated, and may be linear, branched, or cyclic. Specific examples thereof include, but are not limited to, those similar to those exemplified as the hydrocarbyl group of R¹ and R² in the description of the formula (1). When e3 is 2, 3, or 4, R⁴³s may be the same as or different from each other.

When e3 is 2, 3, or 4, a plurality of R⁴³s may be bonded to each other to form a ring together with carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of the hydrogen atoms of the ring may be replaced with a group containing a heteroatom such as an oxygen atom, a sulfur atom, a nitrogen atom, or a halogen atom, some of the (—CH₂-)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

Specific examples of the anion of the repeat unit c2 include, but are not limited to, those listed below. Note that, in the following formulas, R^{A} is as defined above, and Me is a methyl group.

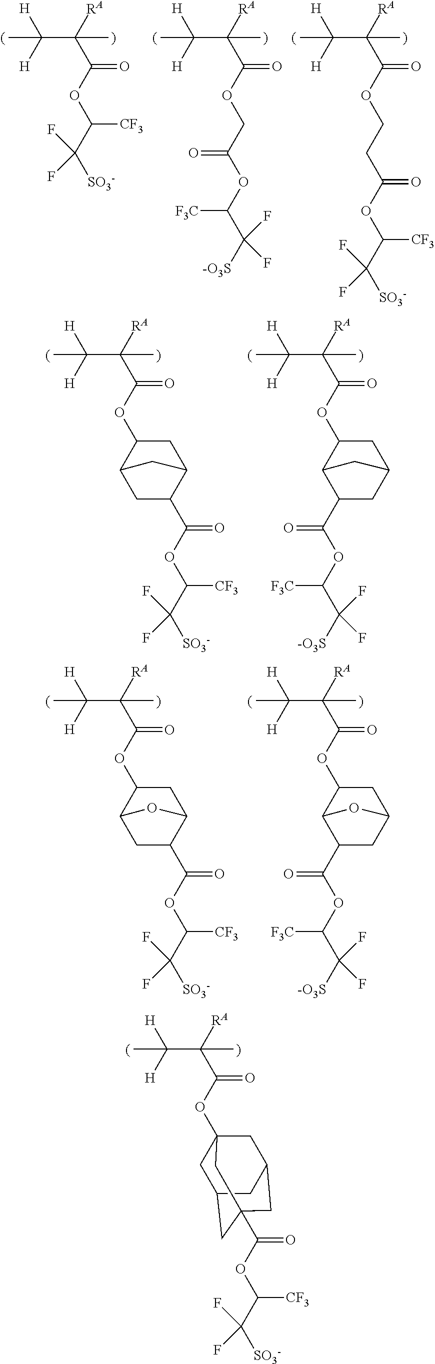

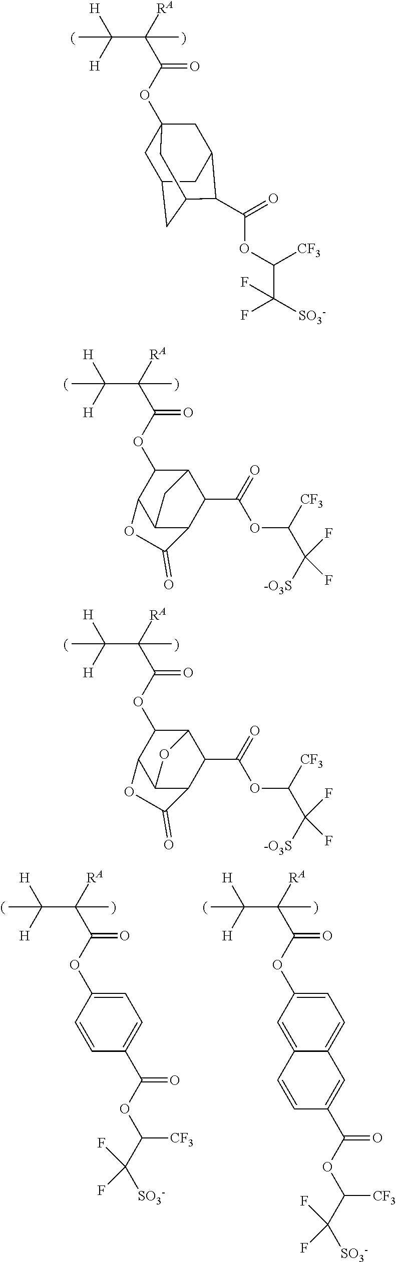

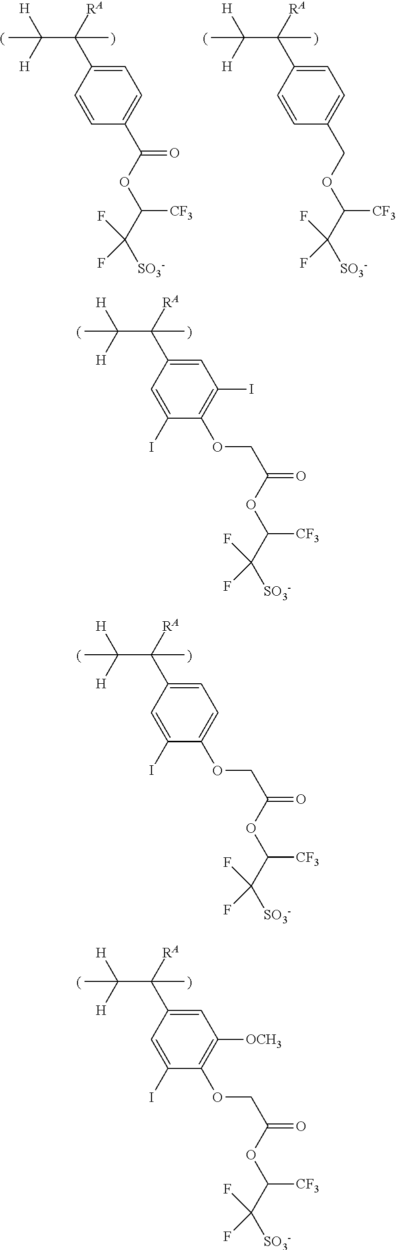

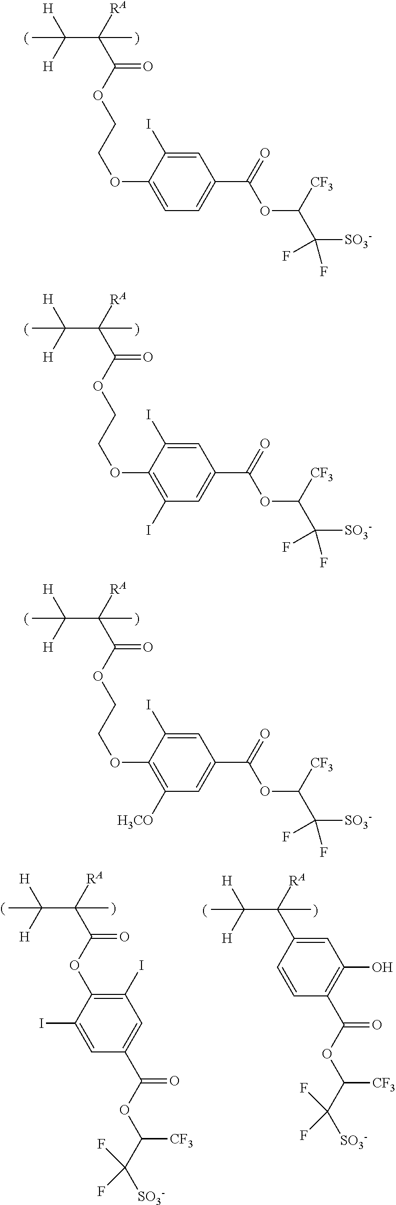

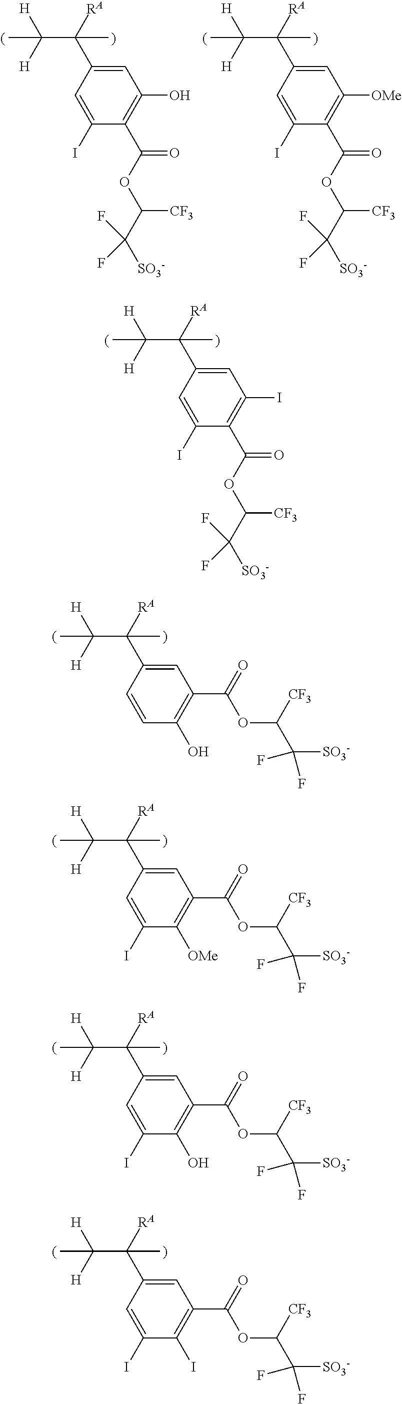

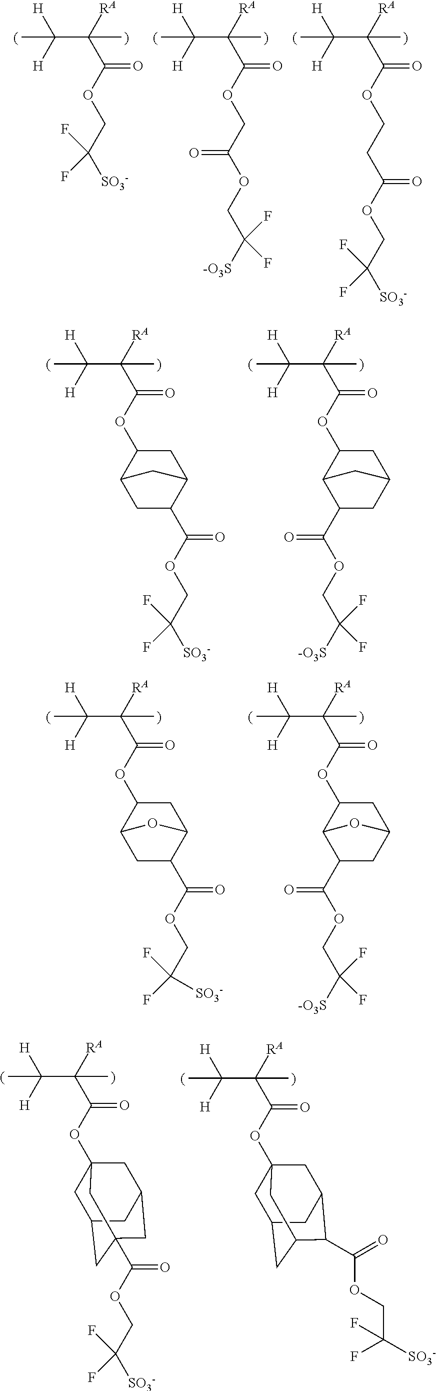

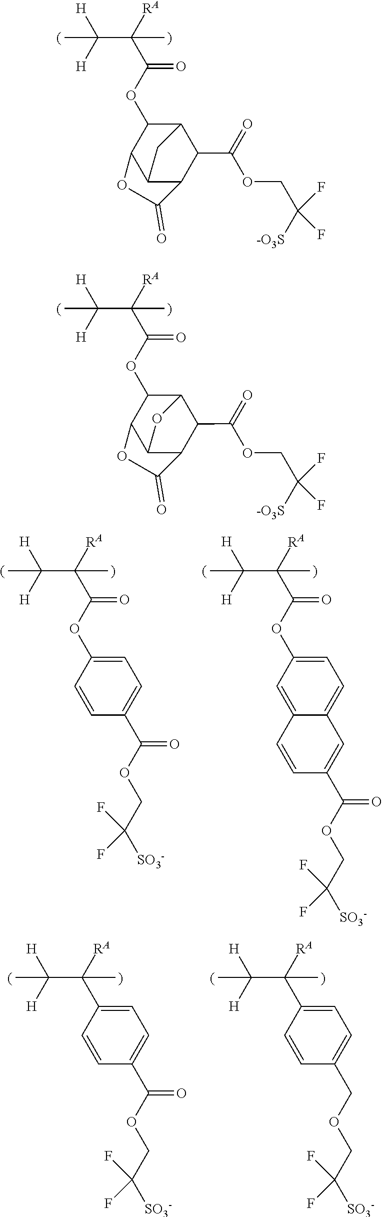

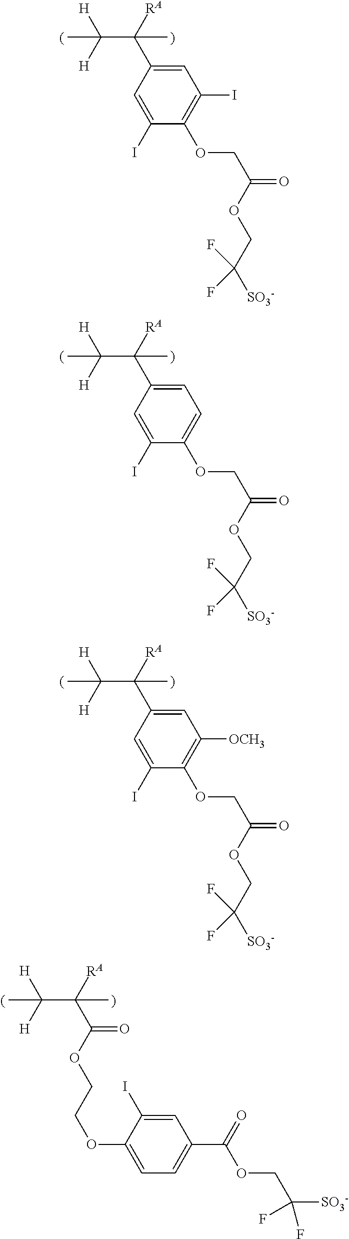

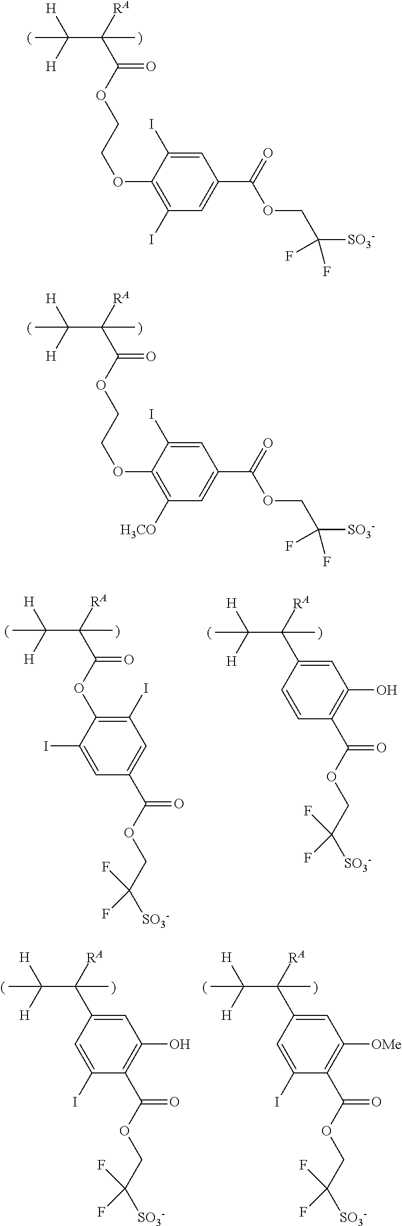

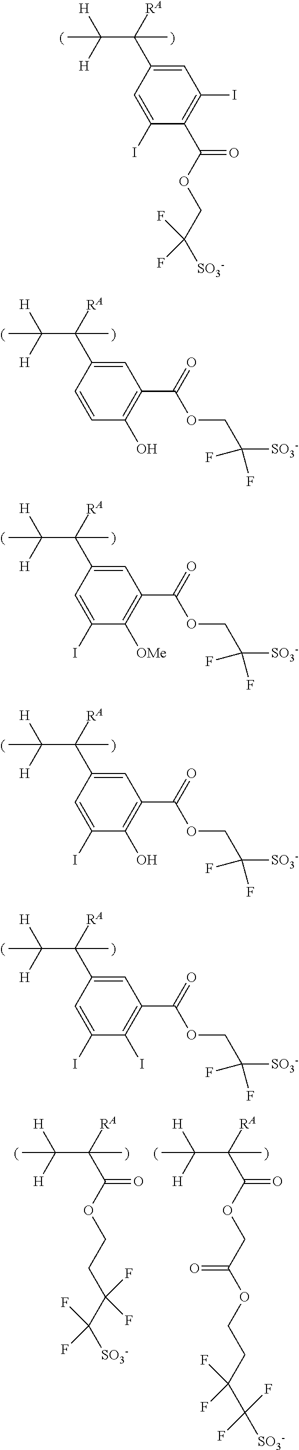

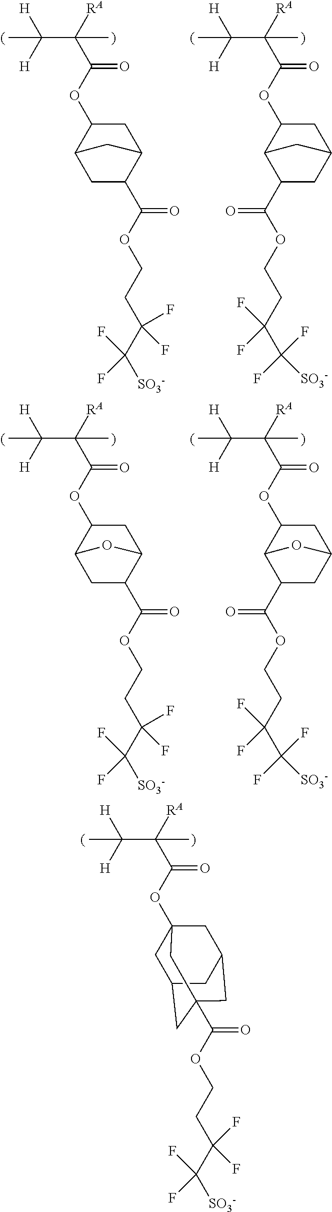

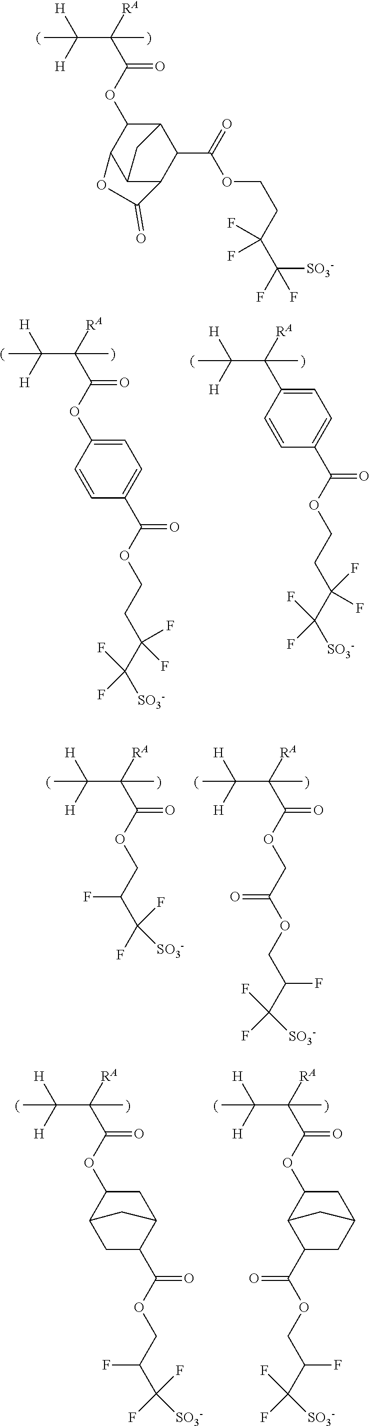

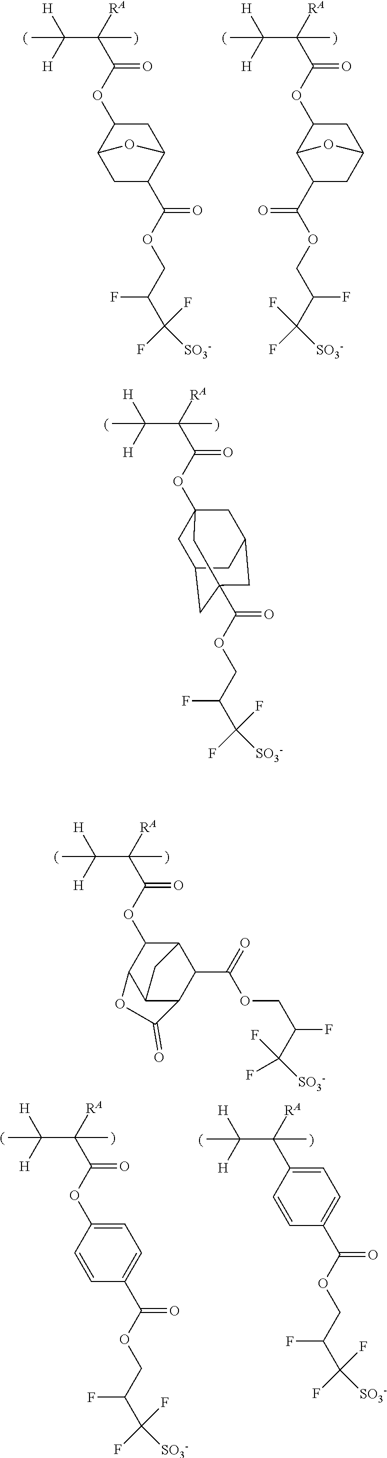

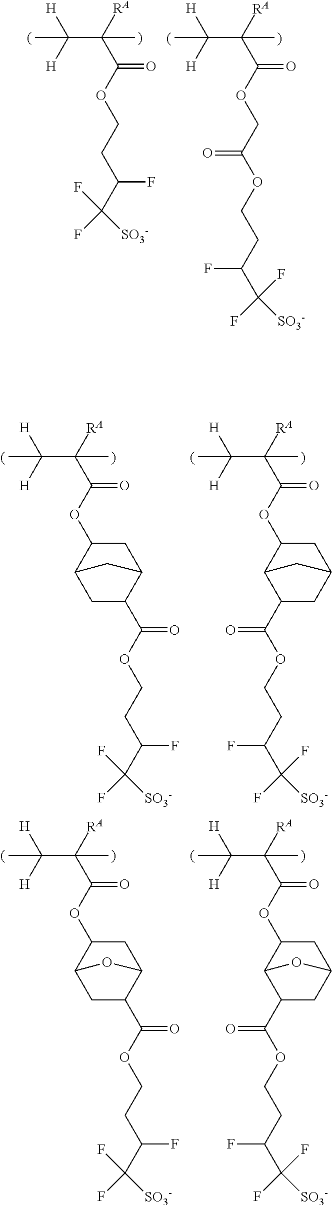

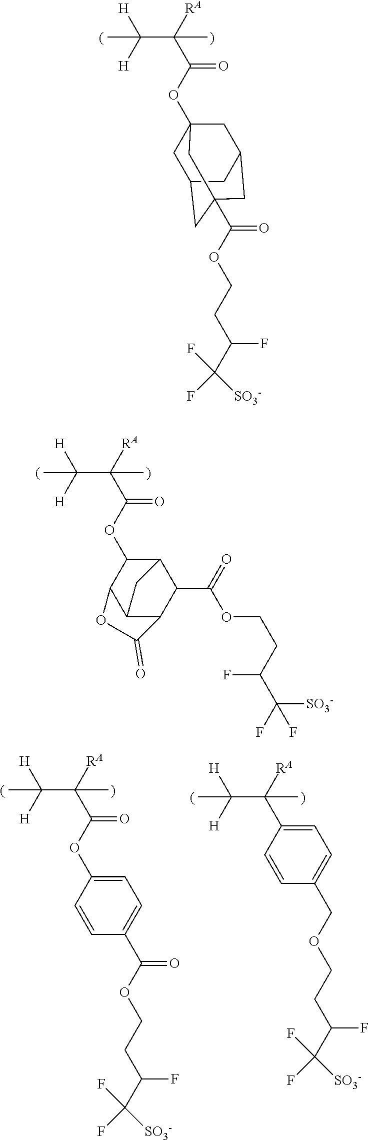

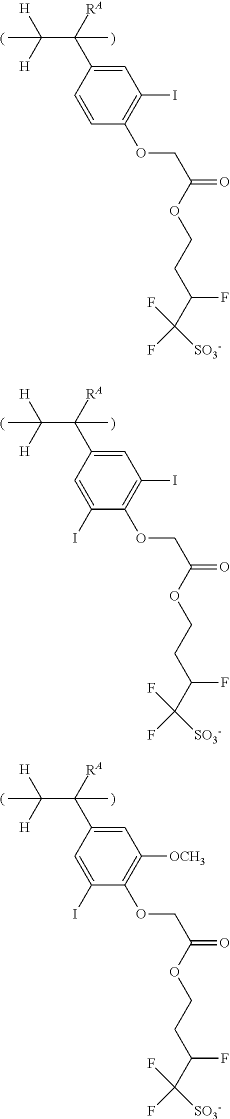

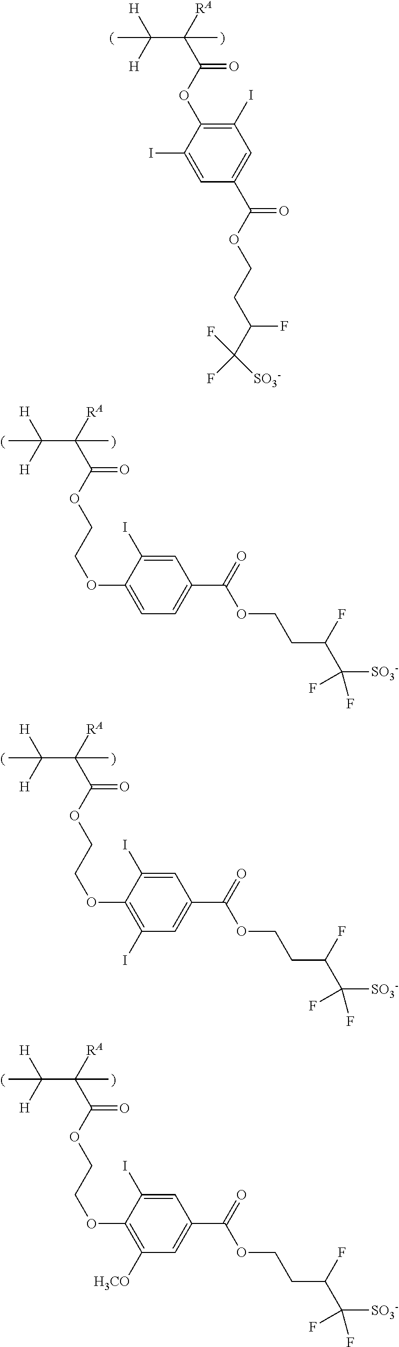

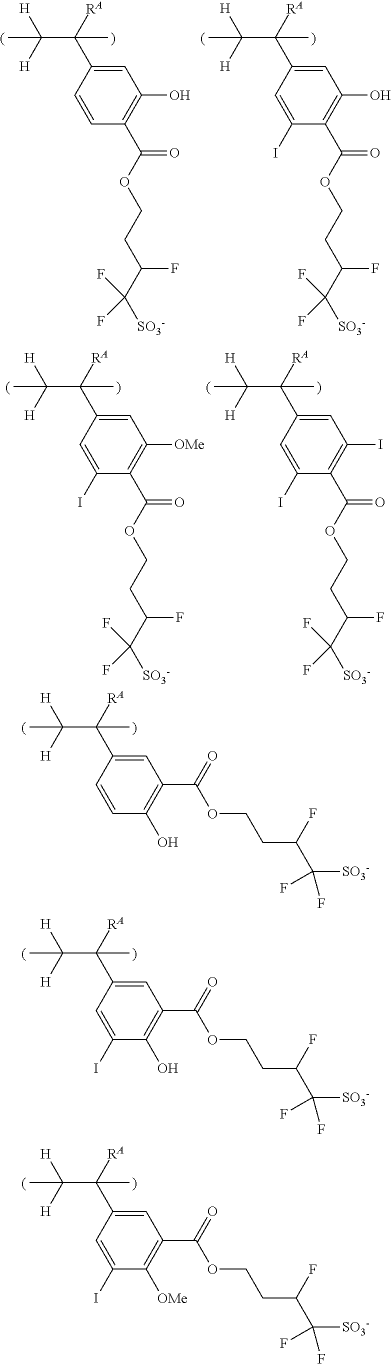

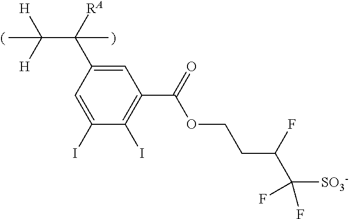

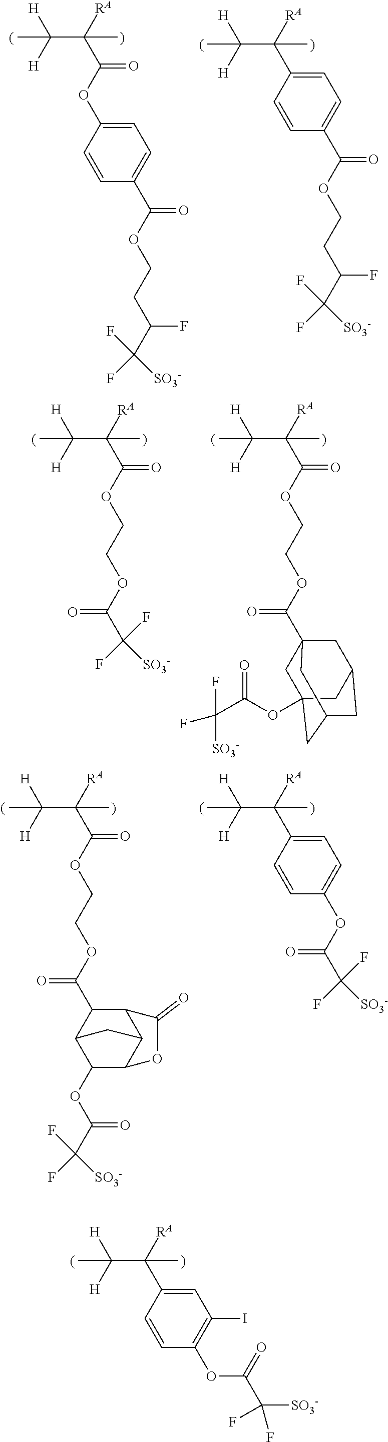

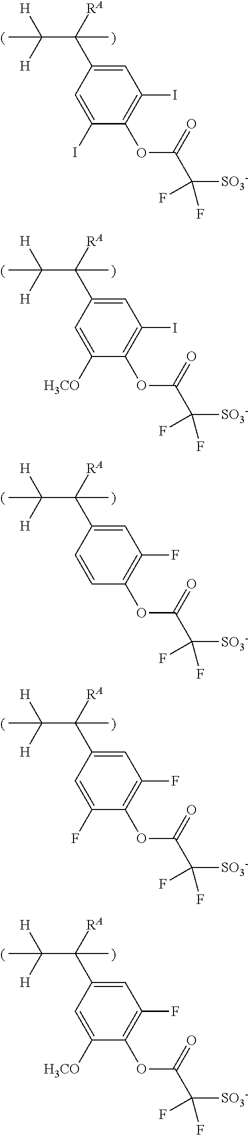

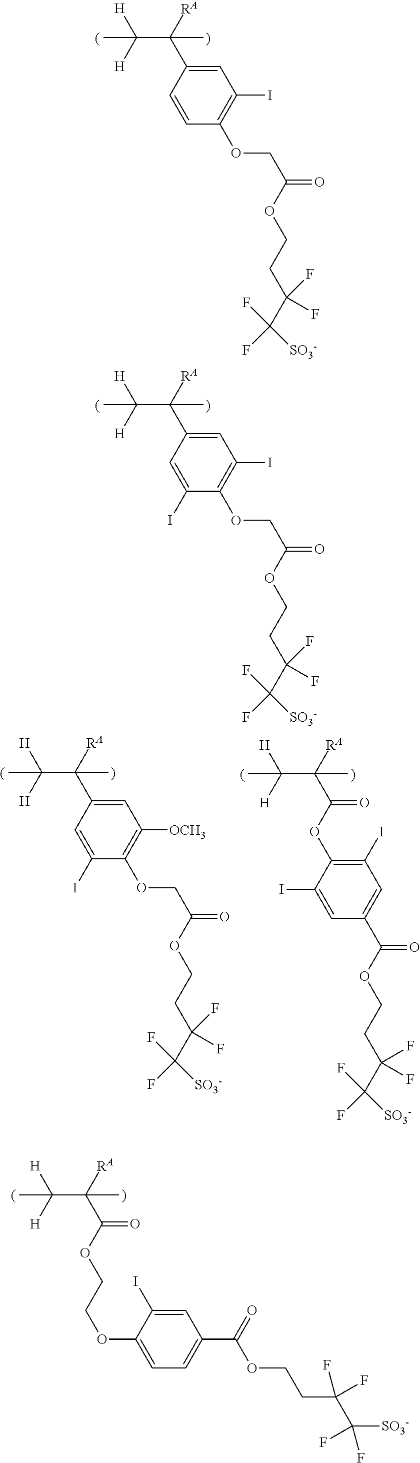

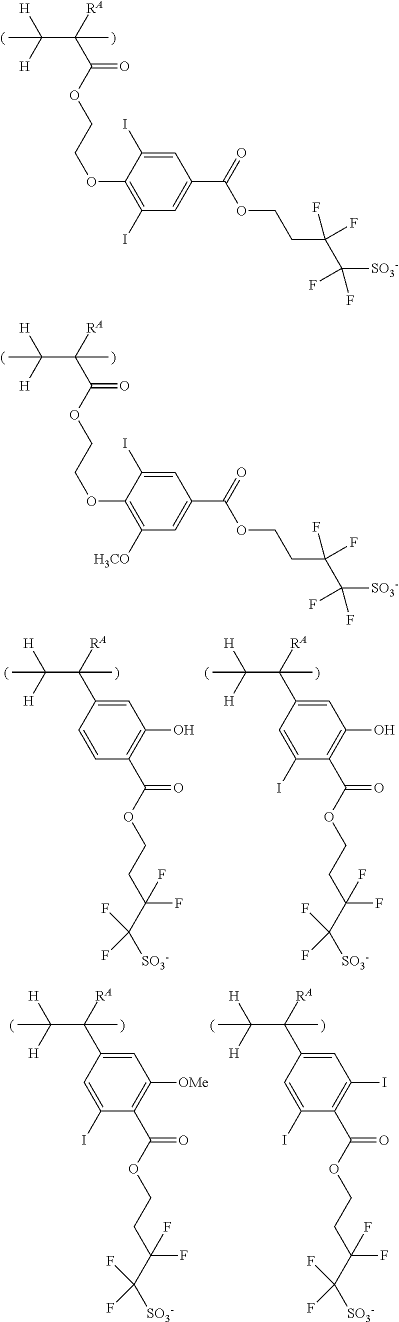

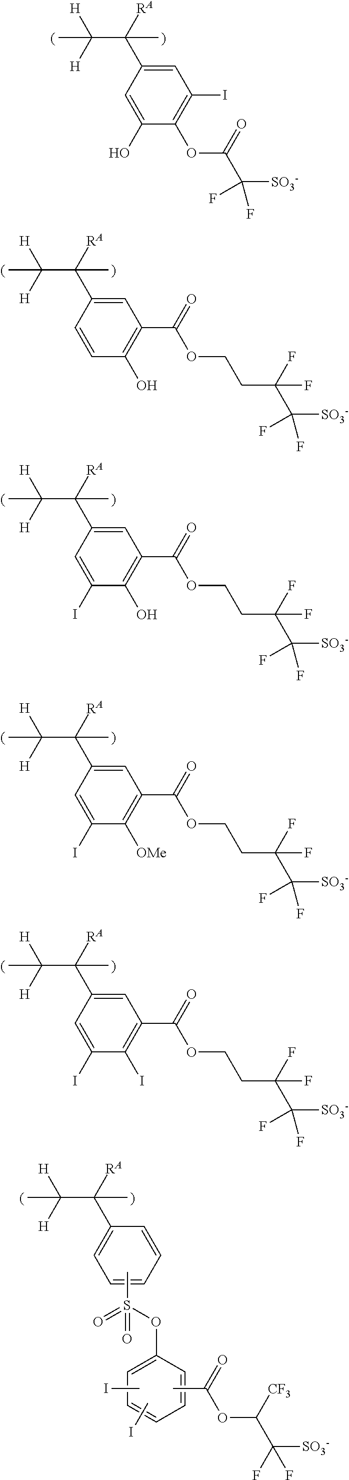

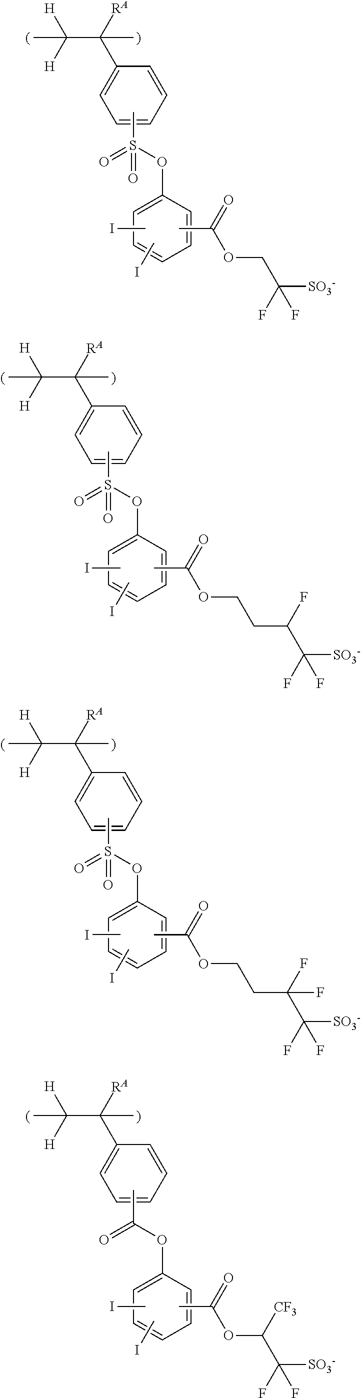

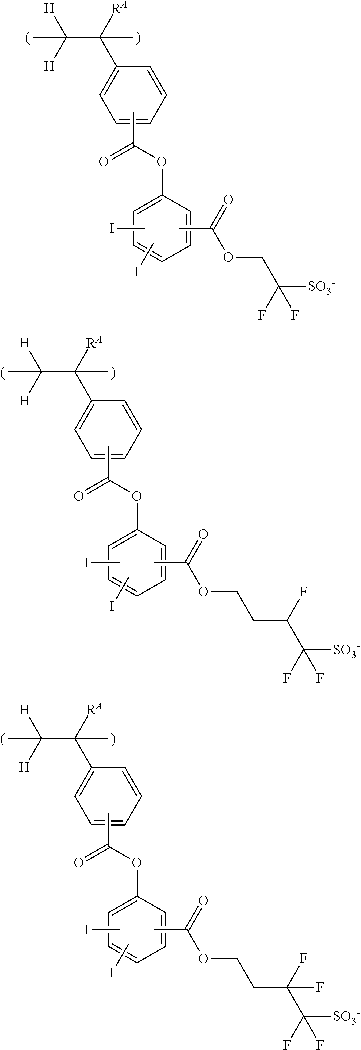

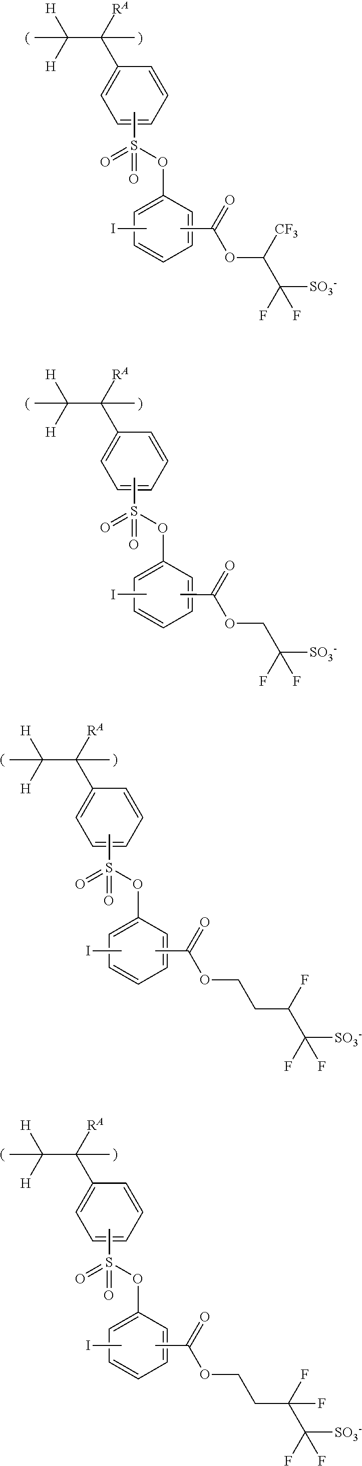

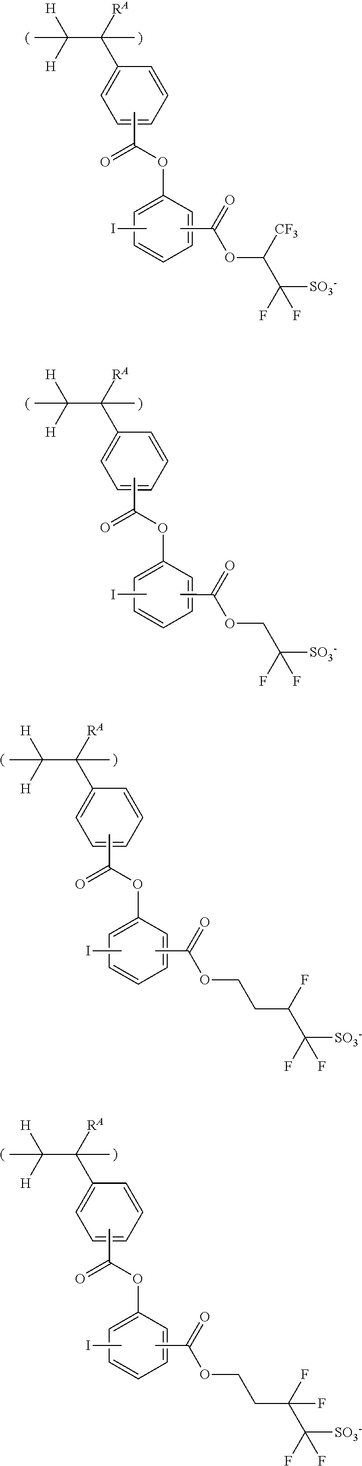

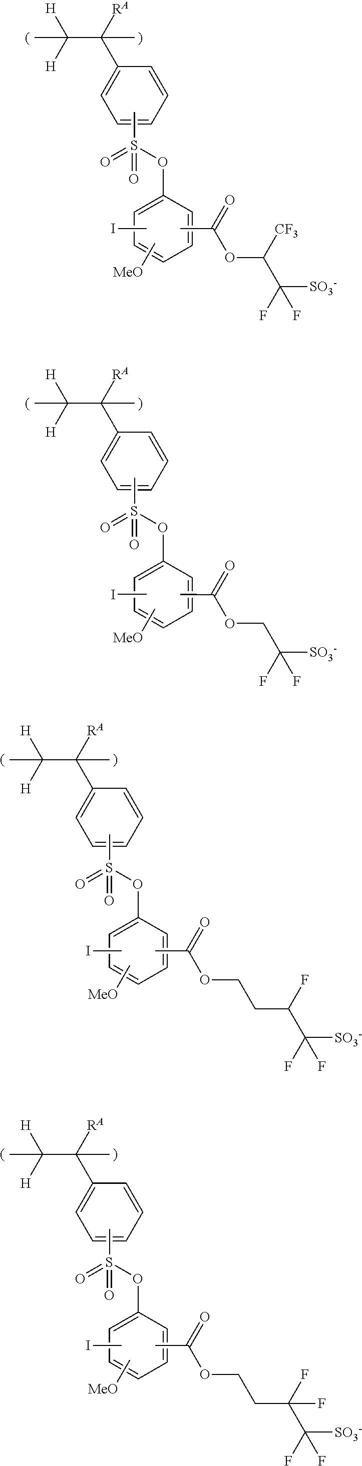

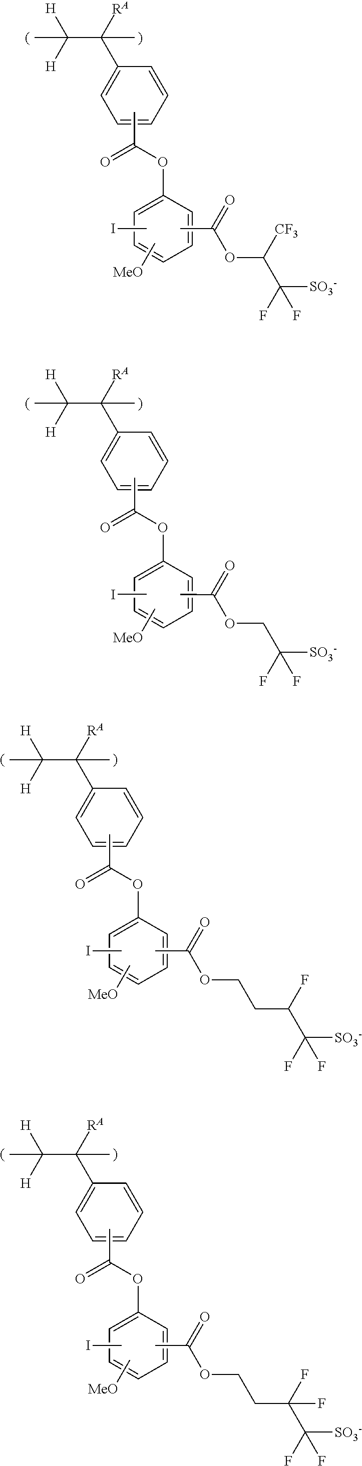

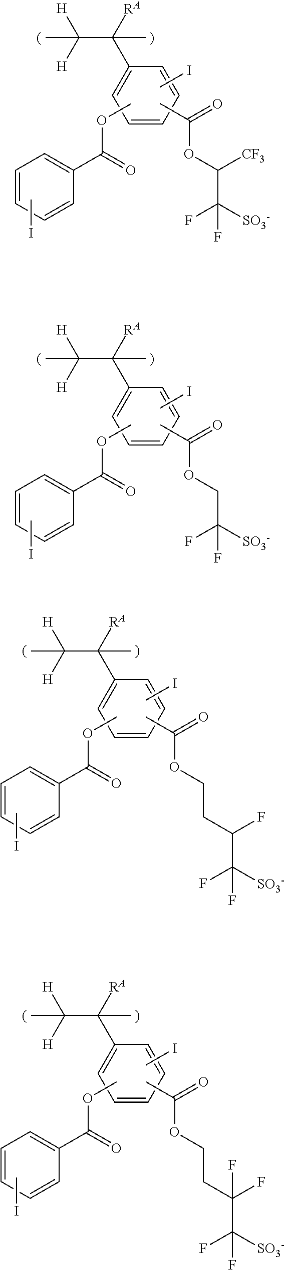

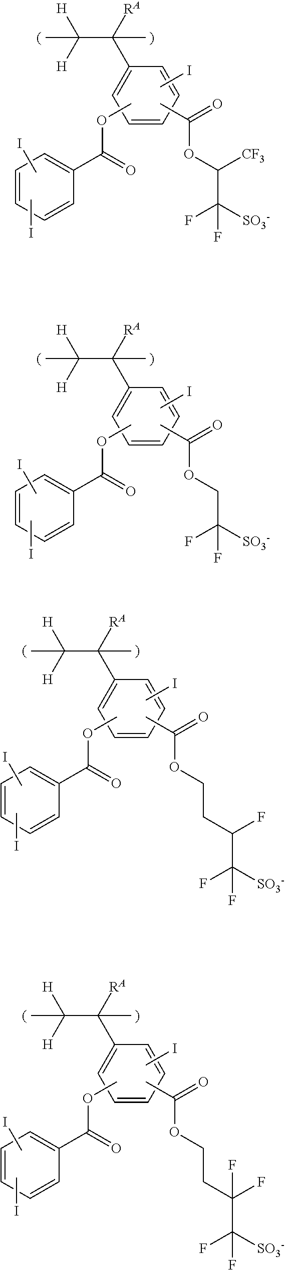

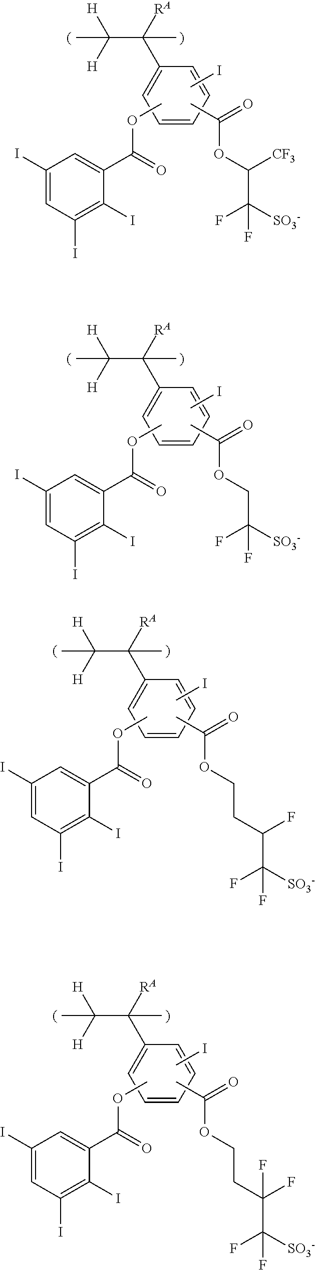

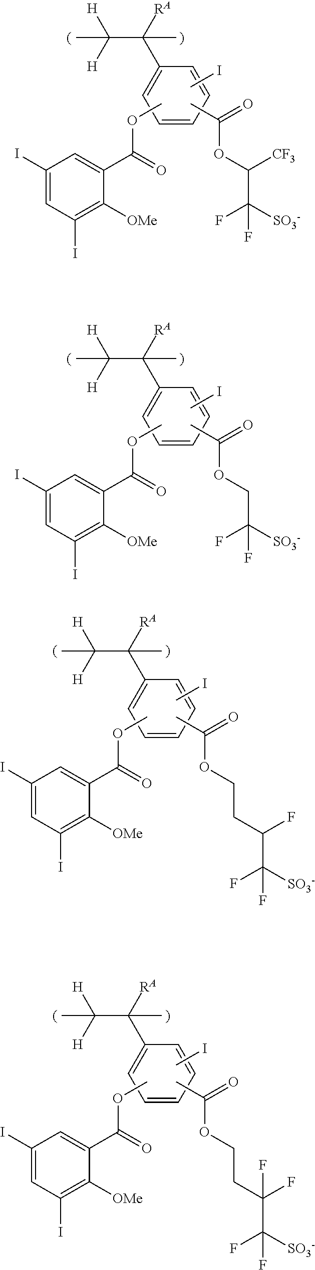

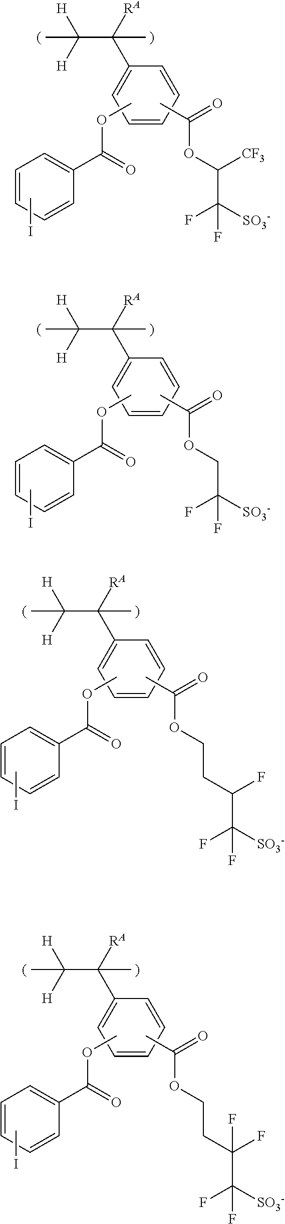

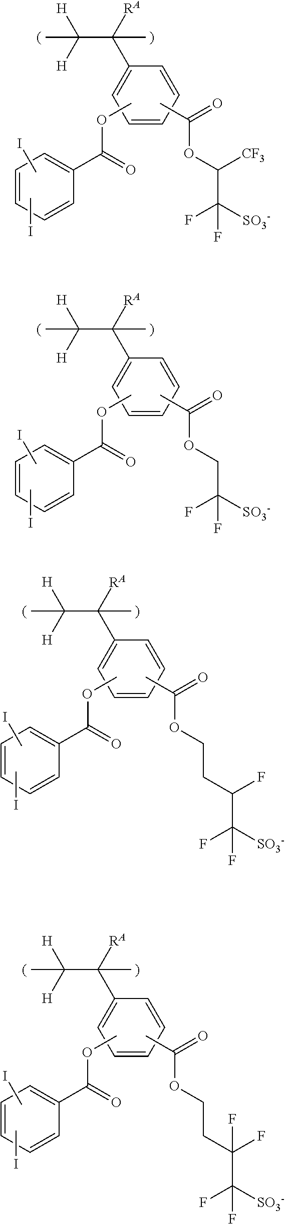

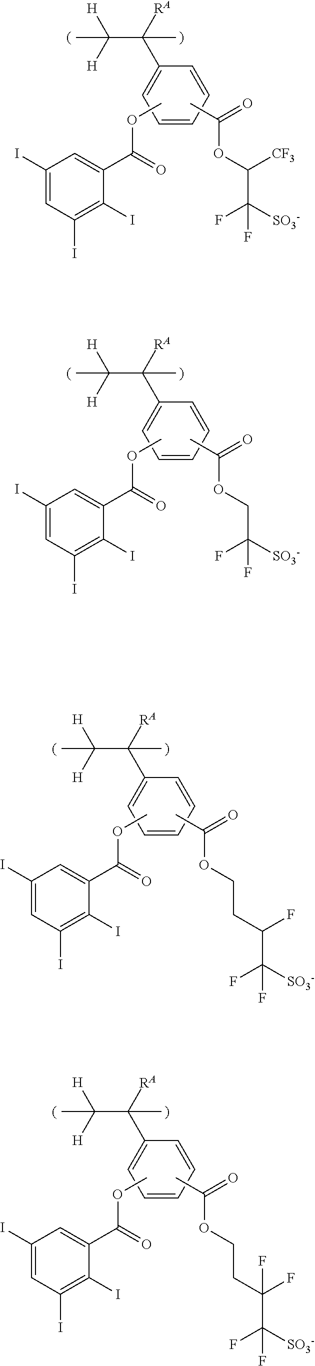

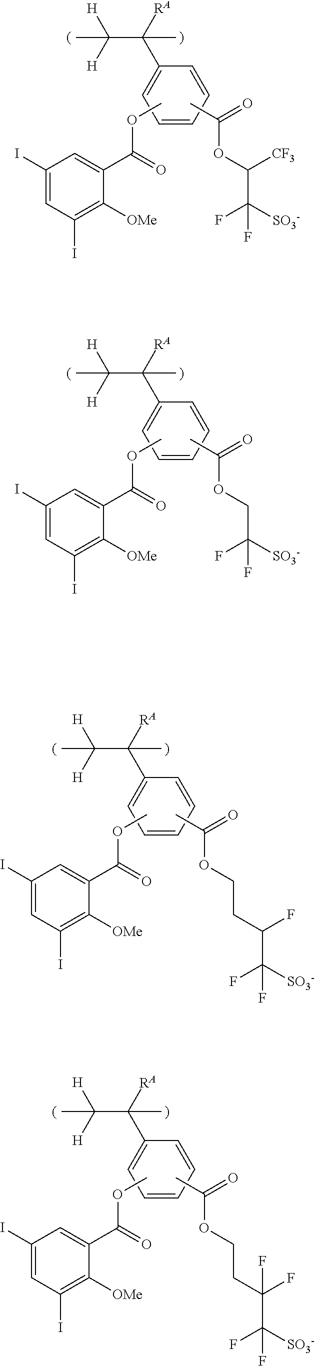

Other specific examples of the repeat unit c2 include those described in paragraphs [0043] and [0044] of WO 2024/176672, paragraphs [0067] to [0070] of WO 2024/176701, paragraphs [0057] and [0058] of WO 2024/190386, paragraphs [0020] to [0023] of JP-A 2024-103465, paragraphs [0067] to [0070] of JP-A 2024-120703, and paragraphs [0075] to [0084] of JP 7520258, paragraphs [0032] to [0047] of JP-A 2024-043941, paragraphs [0385] to [0390] of JP-A 2025-013053, and paragraphs [0062] to [0064] of WO 2025/018169.

Specific examples of the anion of the repeat unit c3 include, but are not limited to, those listed below. Note that, in the following formulas, R^{A} is as defined above.

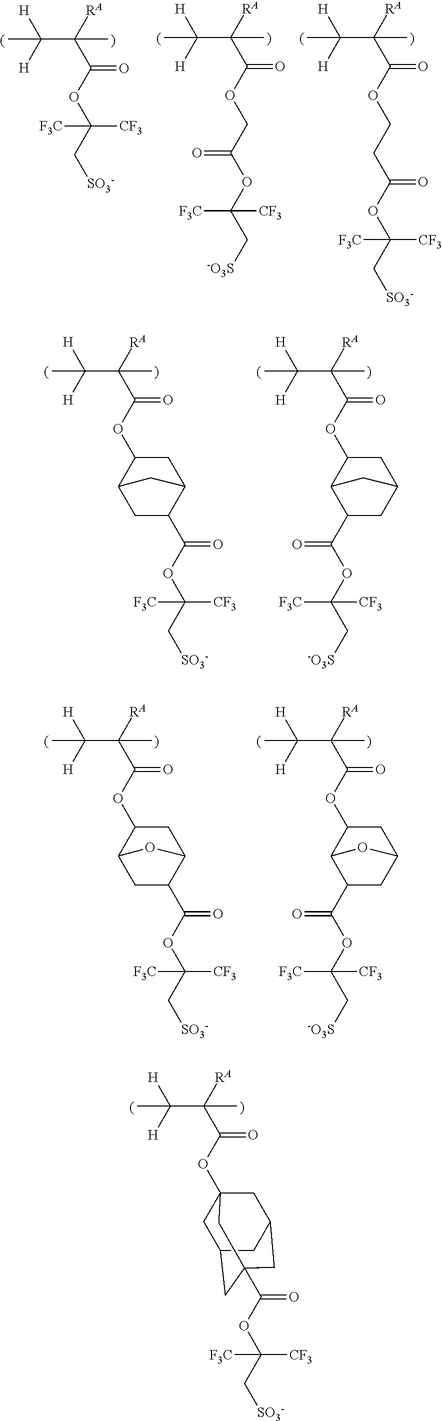

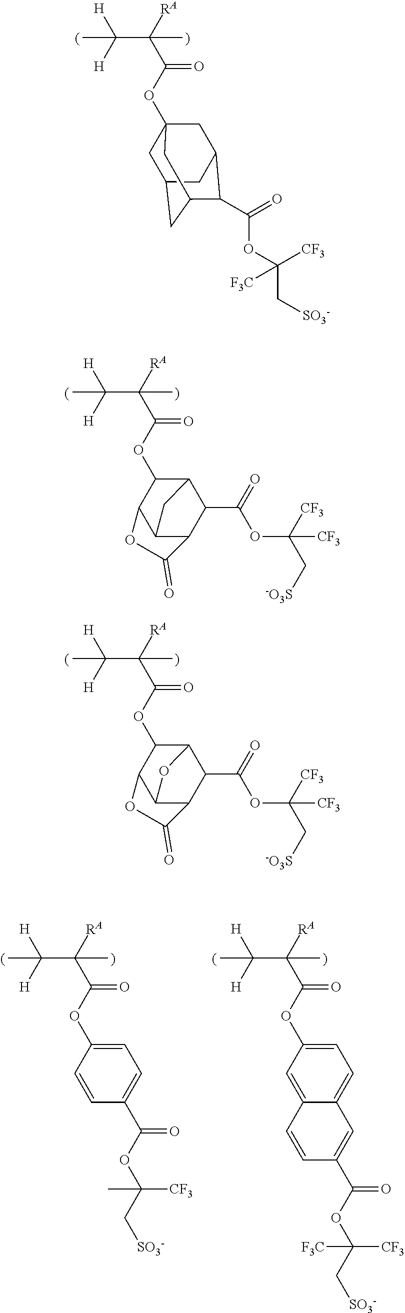

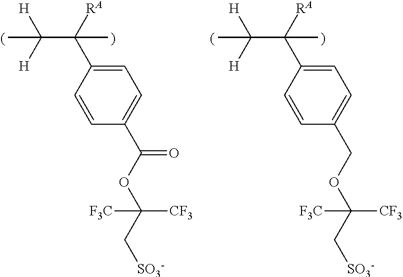

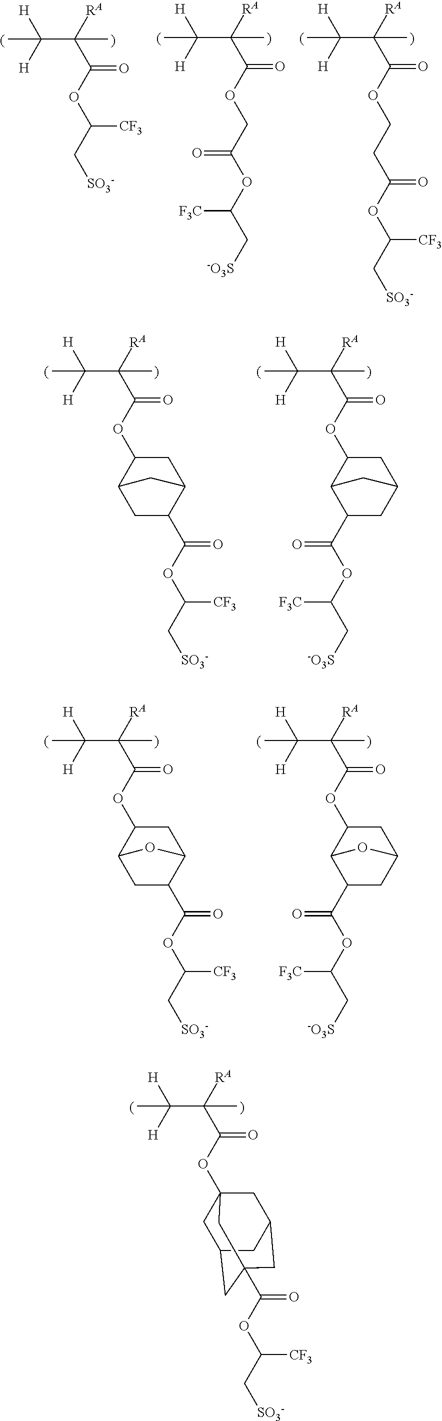

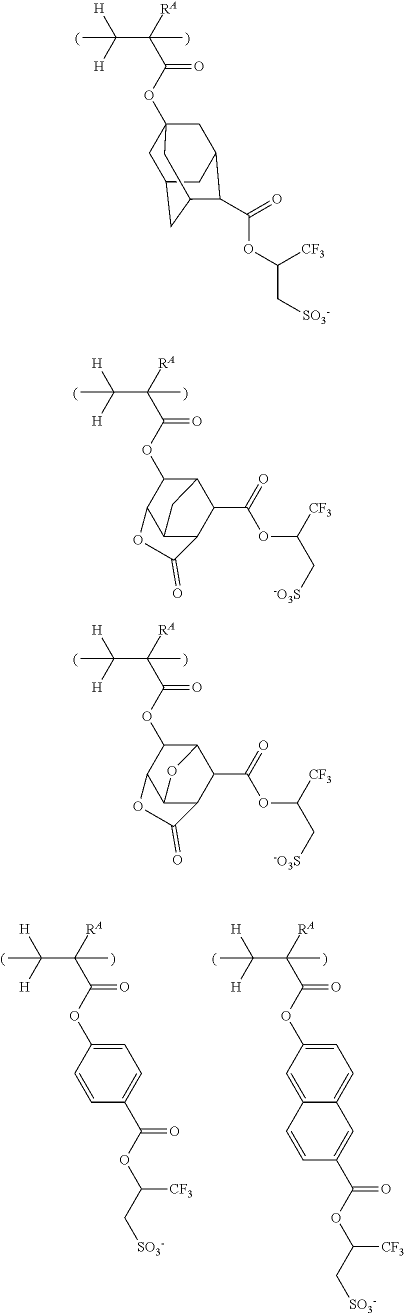

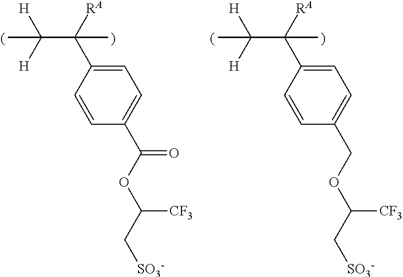

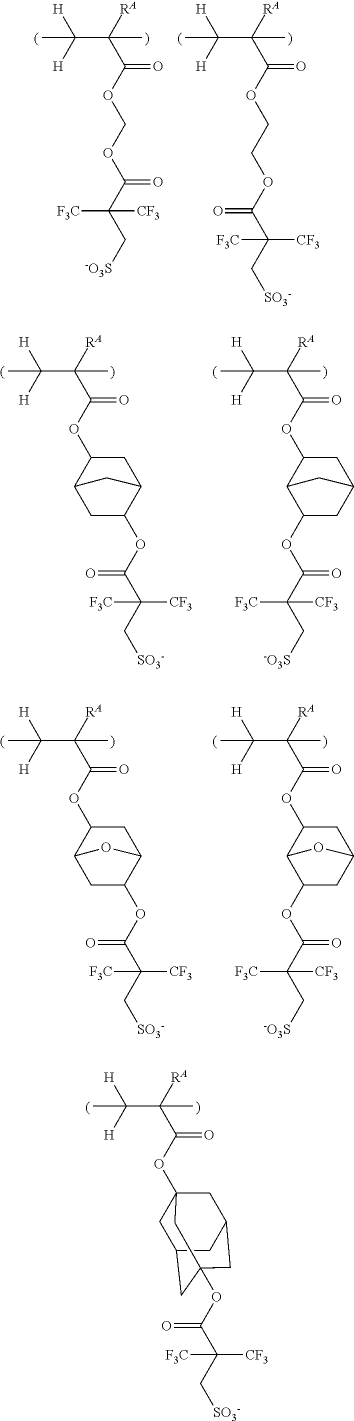

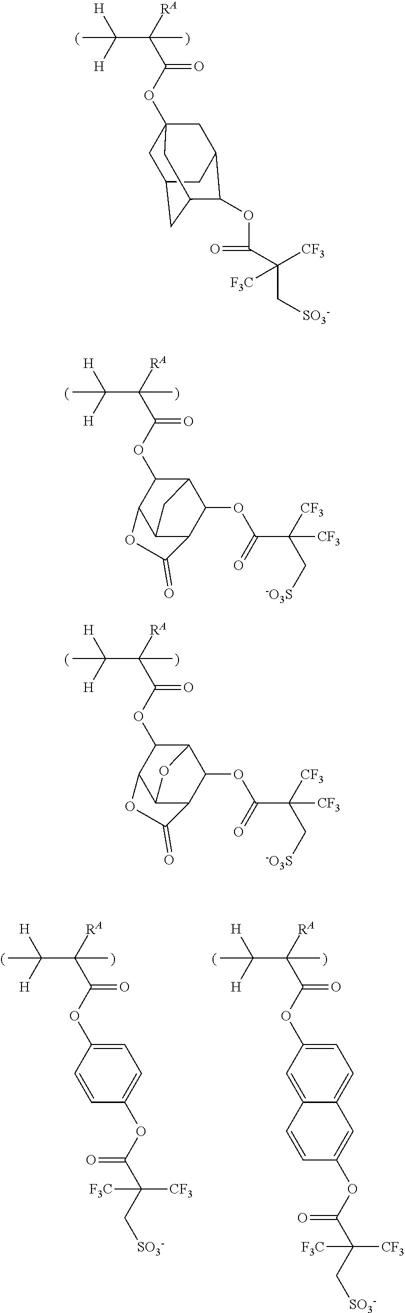

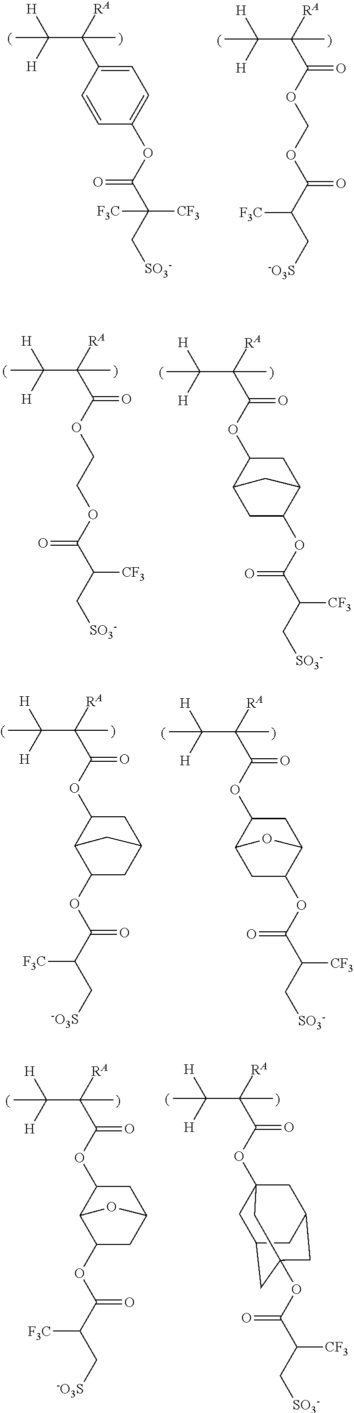

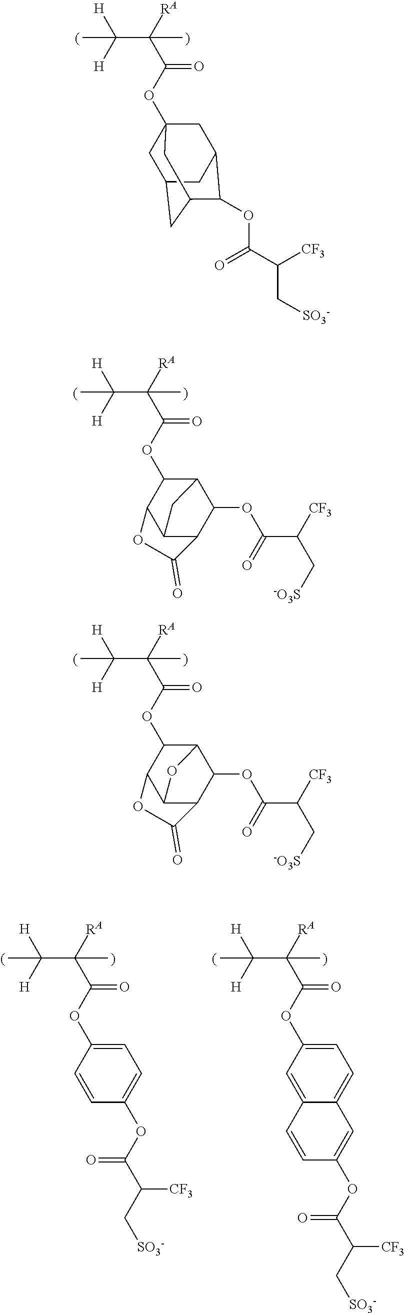

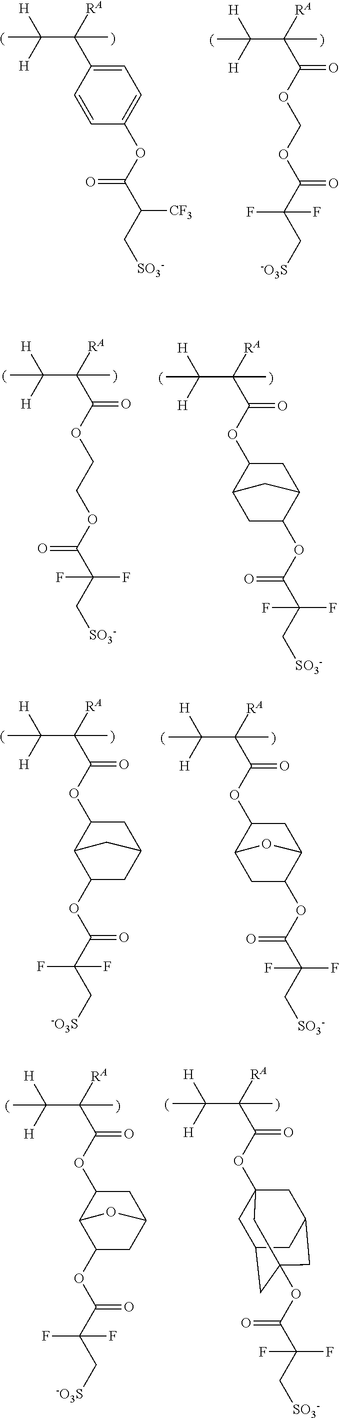

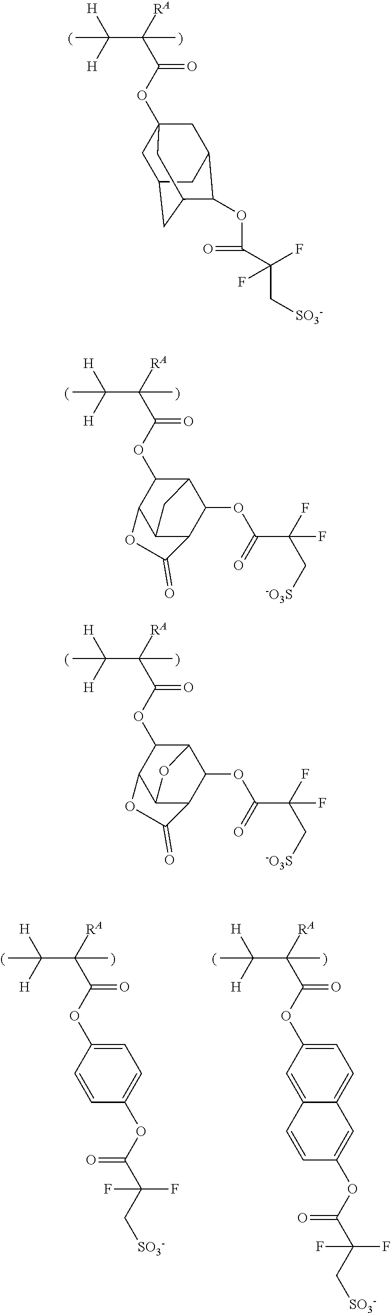

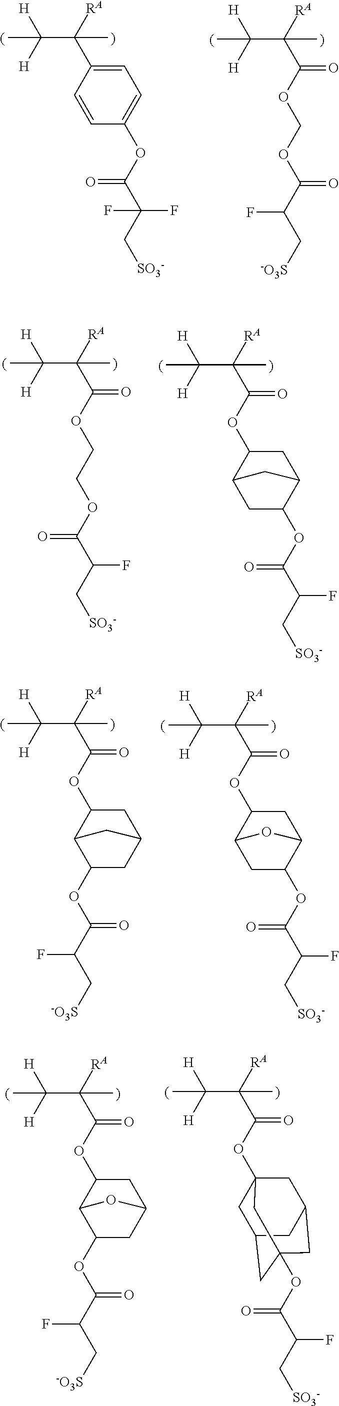

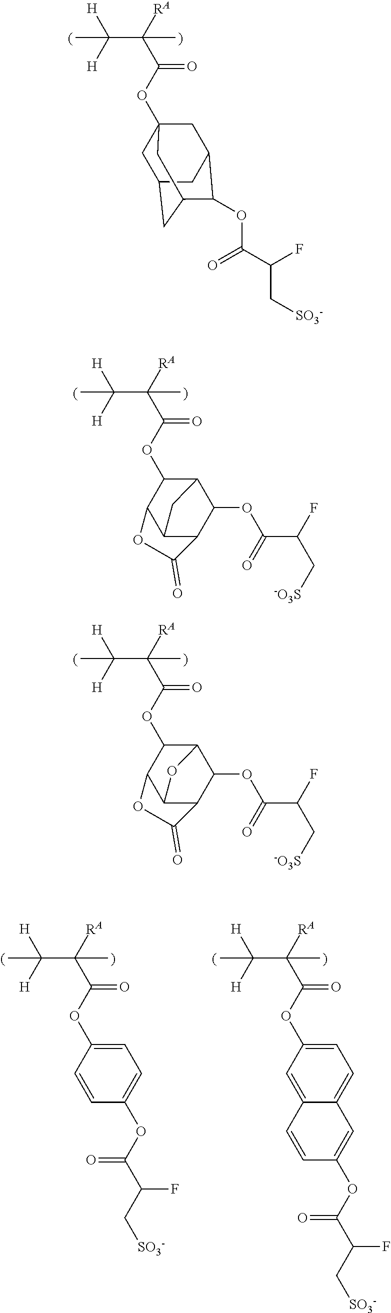

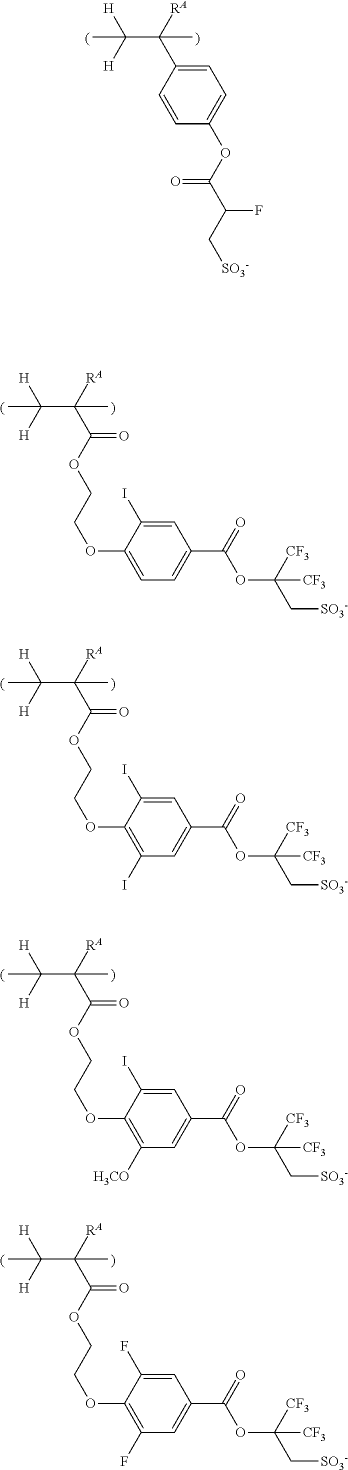

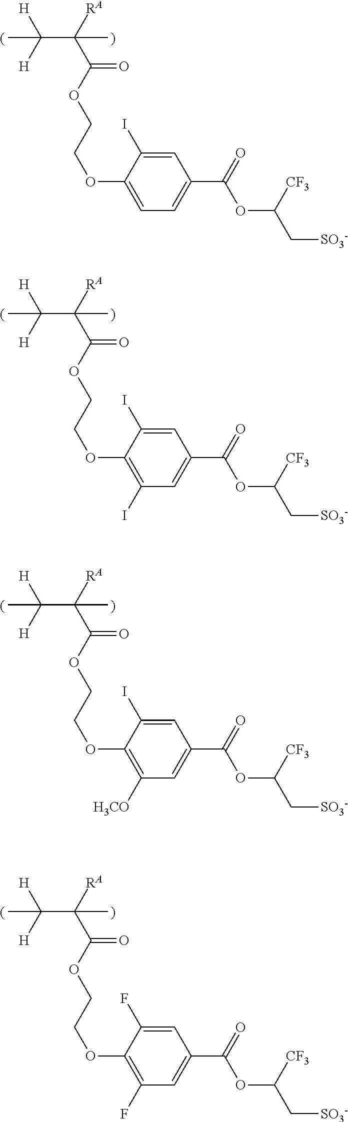

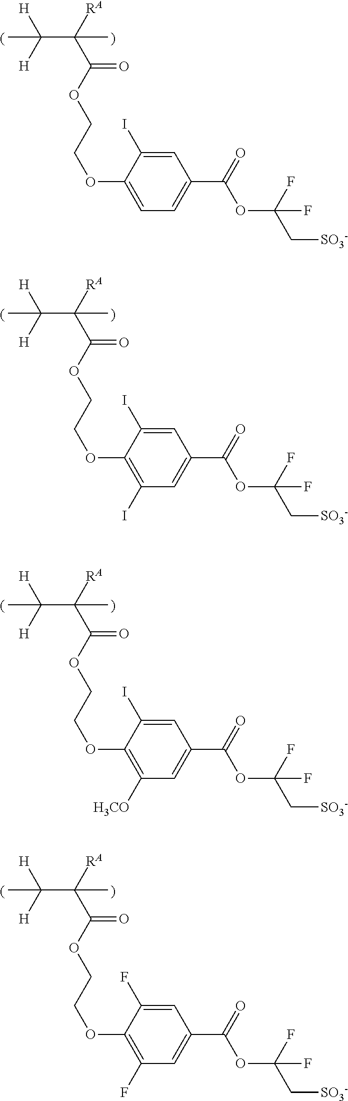

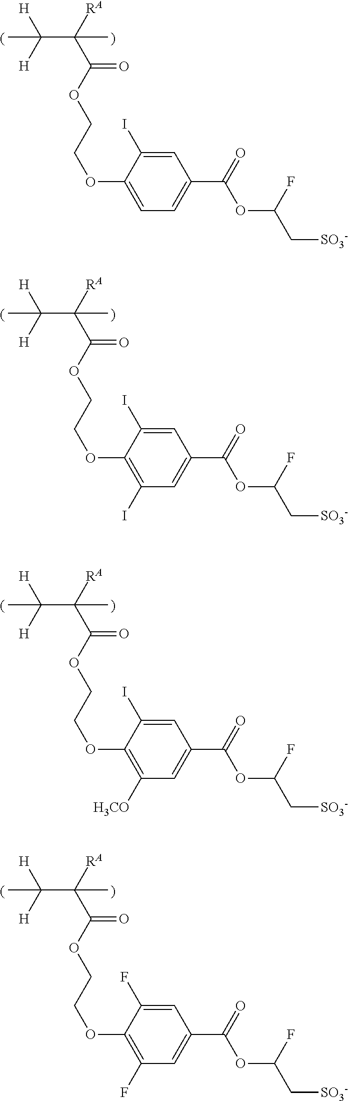

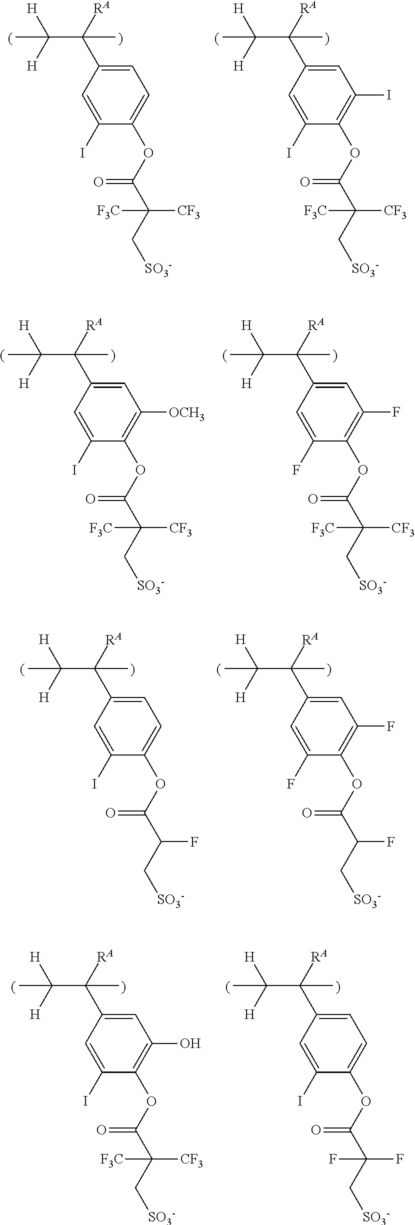

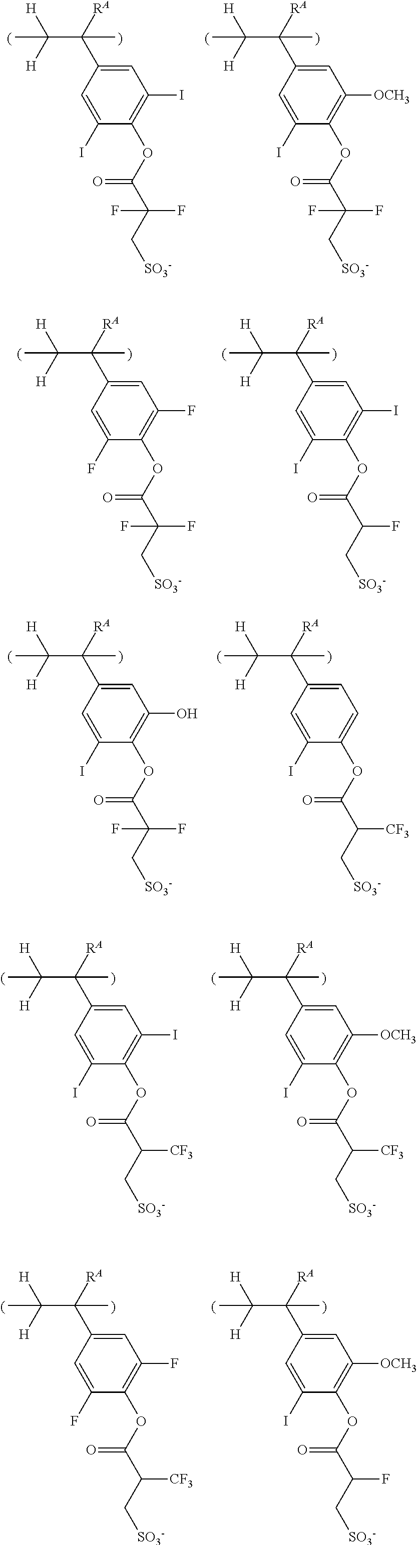

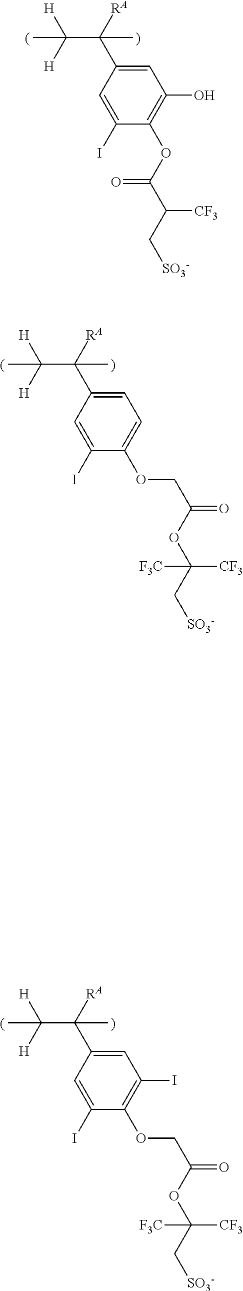

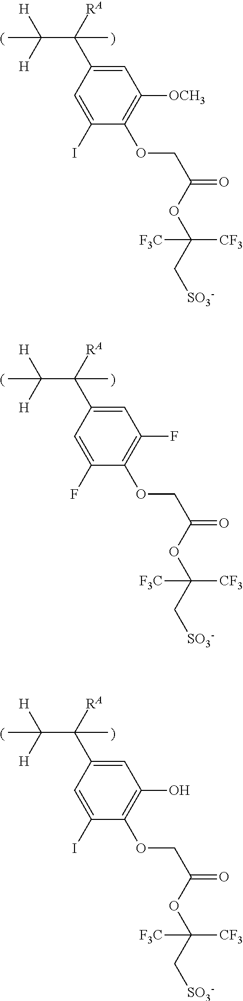

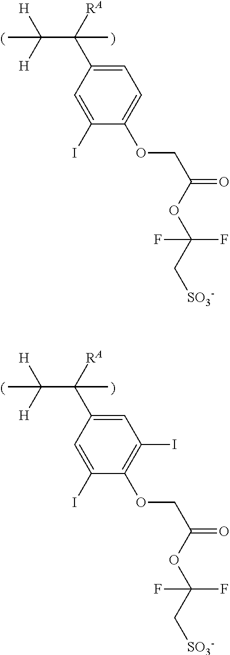

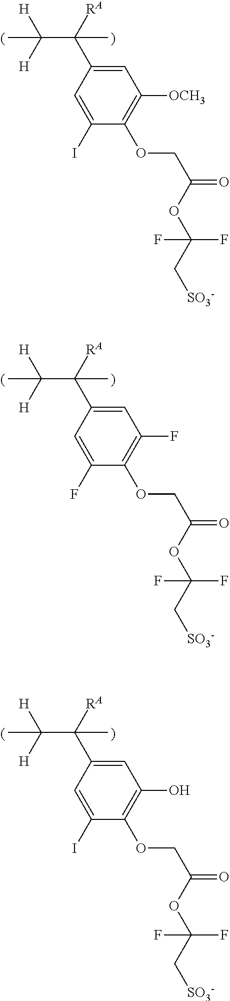

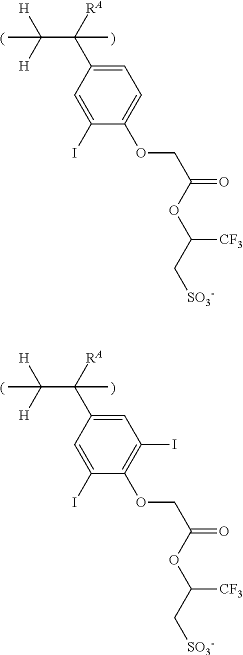

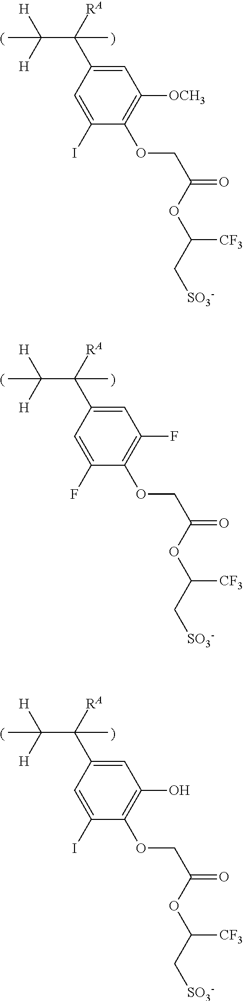

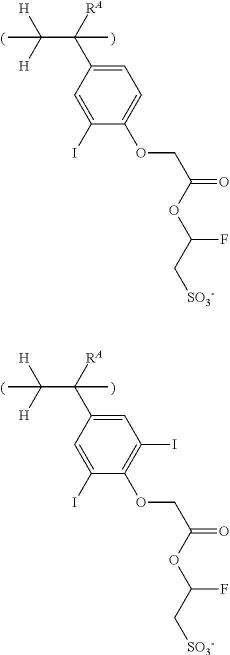

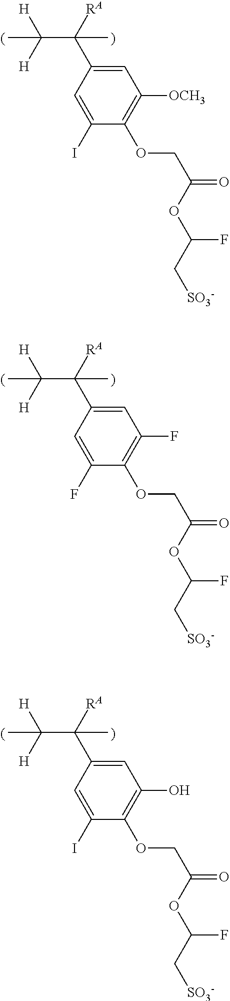

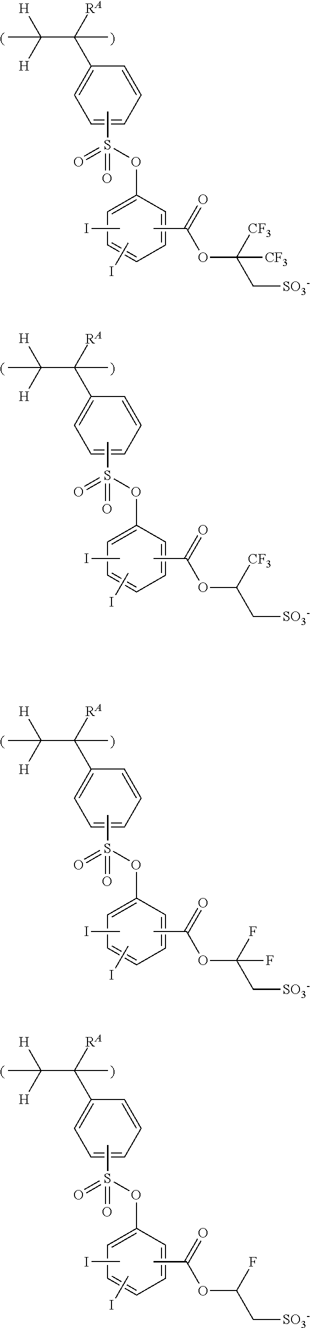

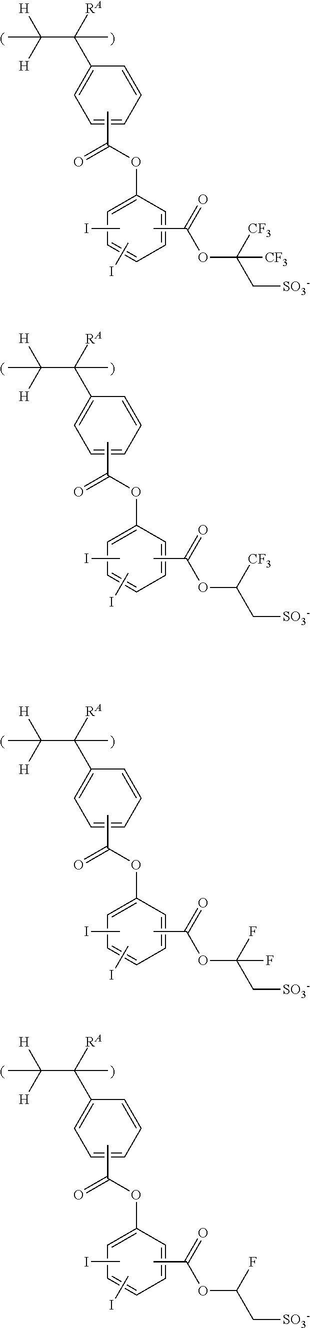

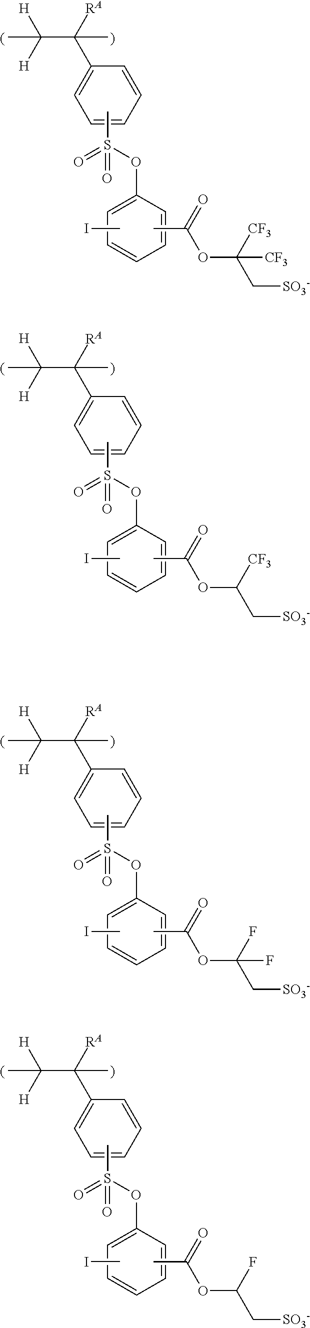

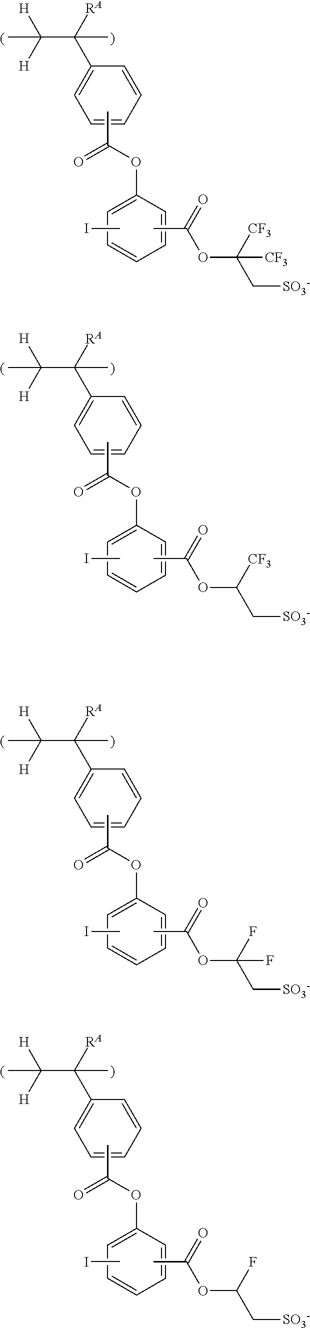

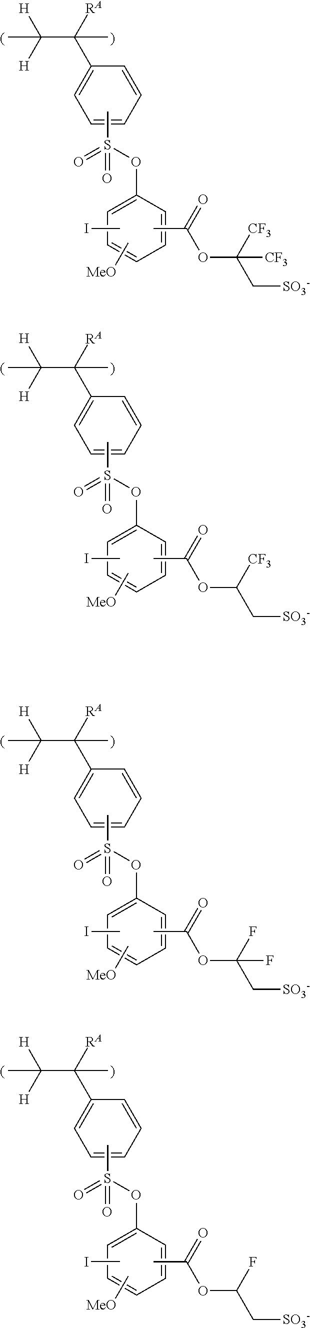

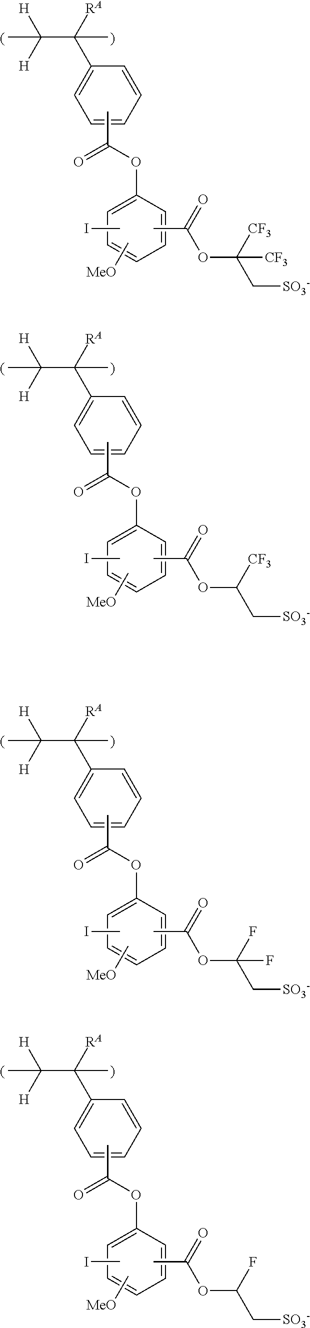

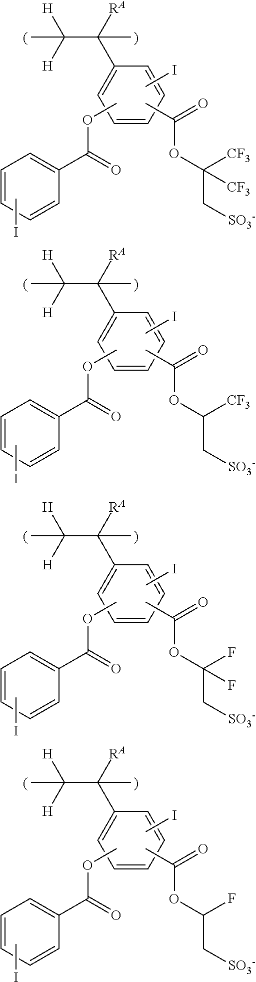

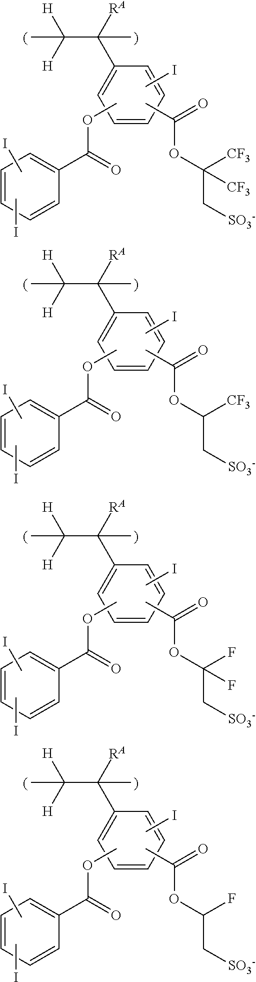

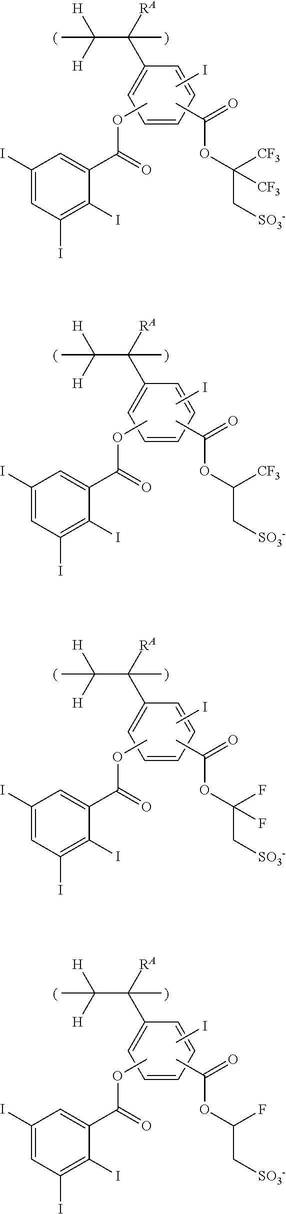

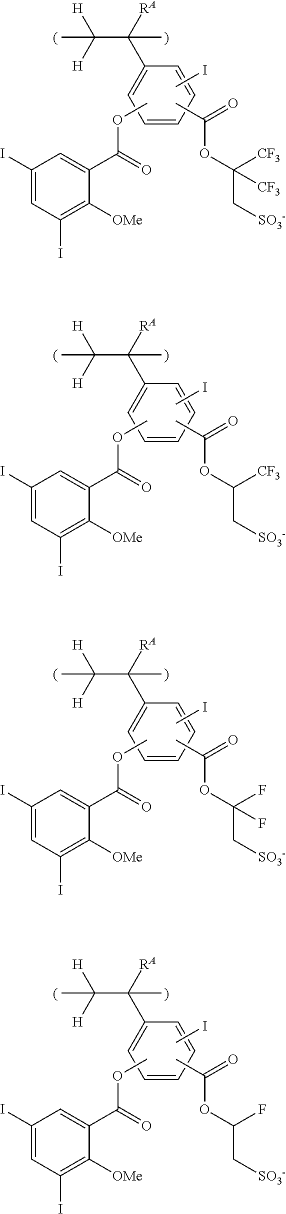

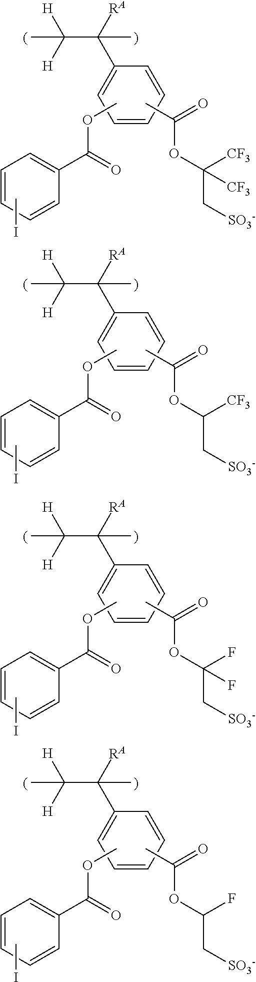

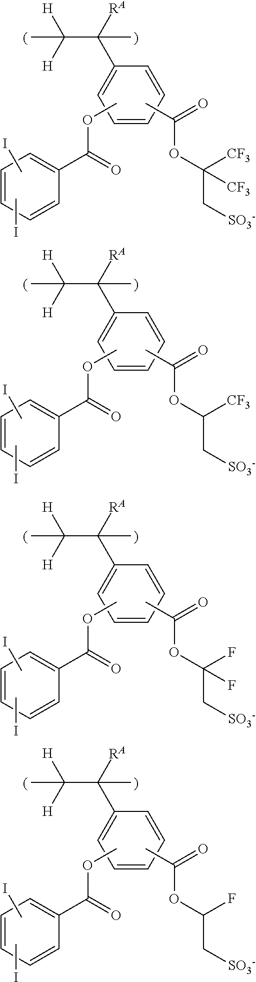

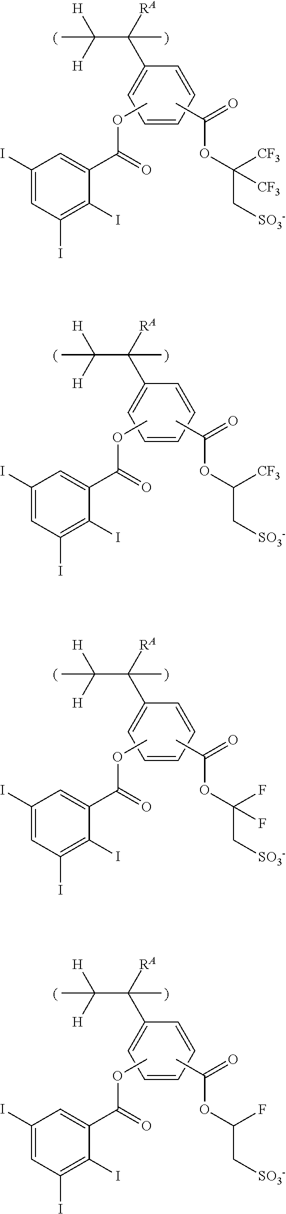

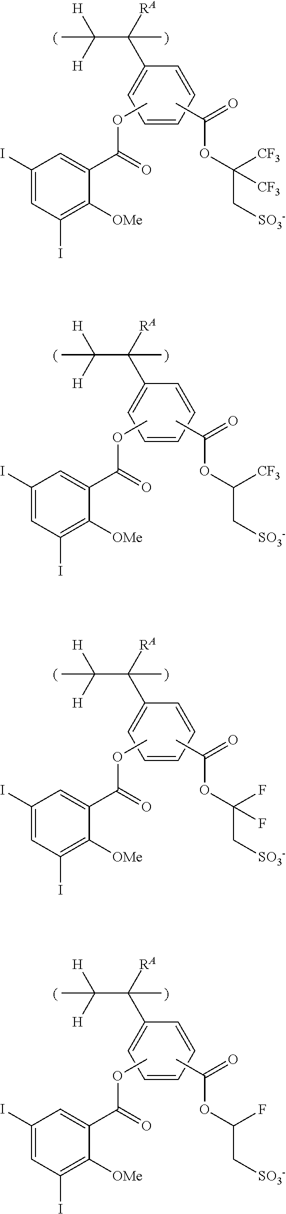

Specific examples of the anion of the repeat unit c4 include, but are not limited to, those listed below. Note that, in the following formulas, R^{A} is as defined above.

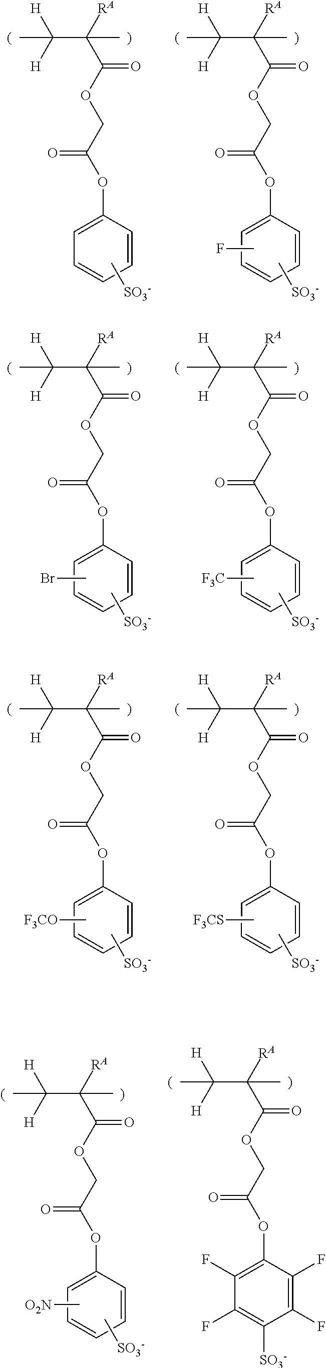

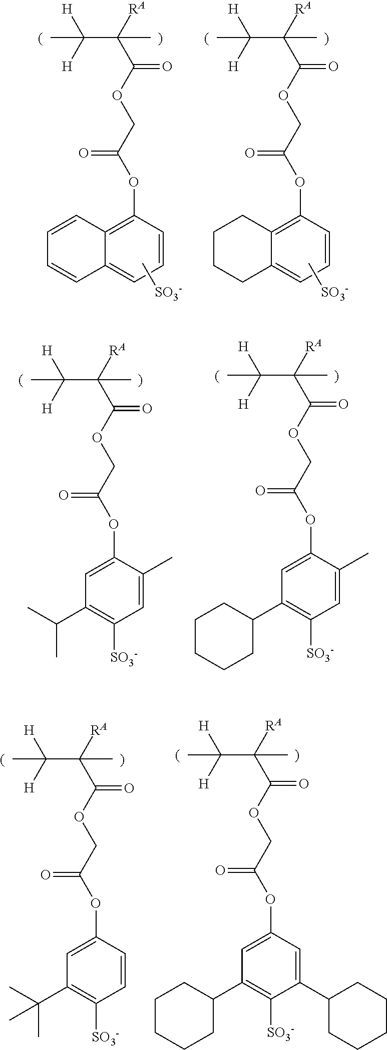

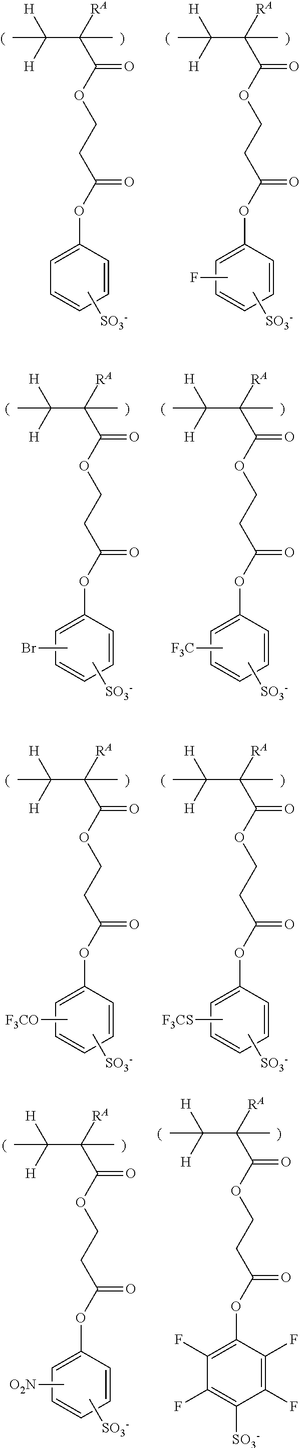

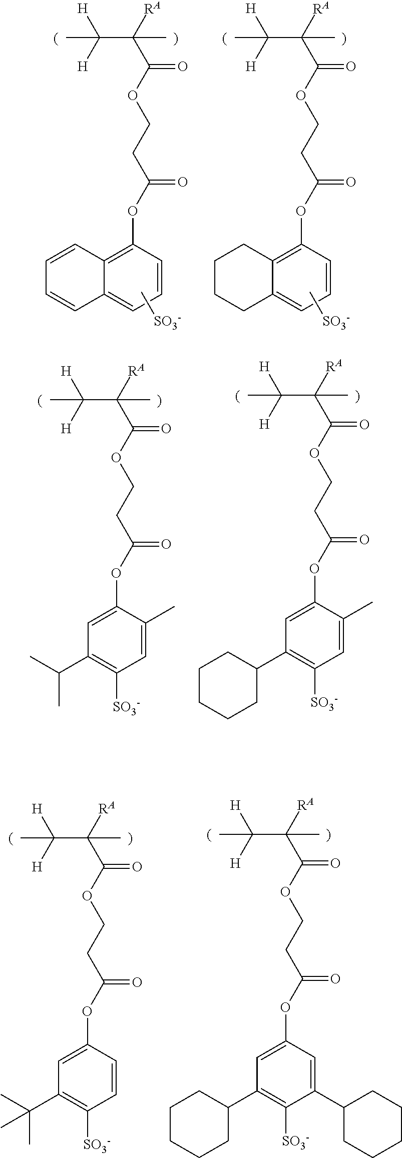

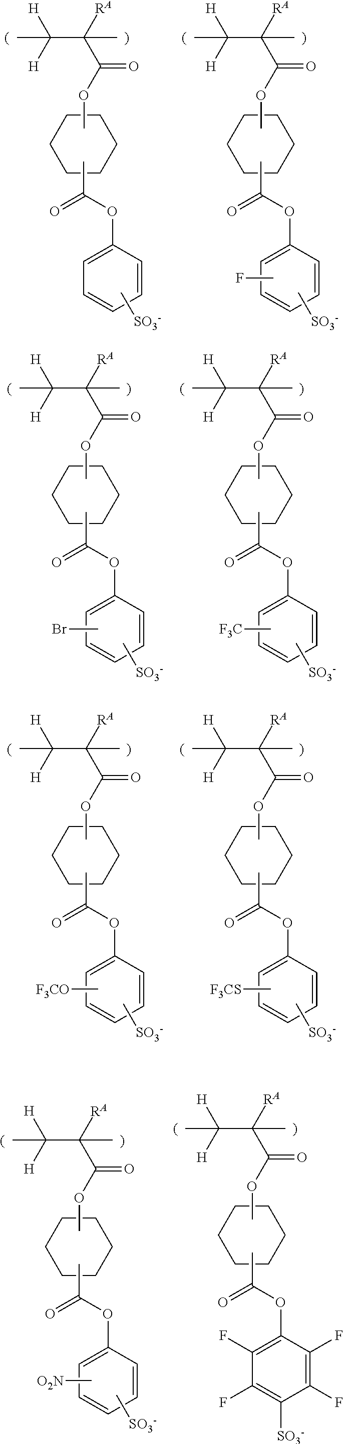

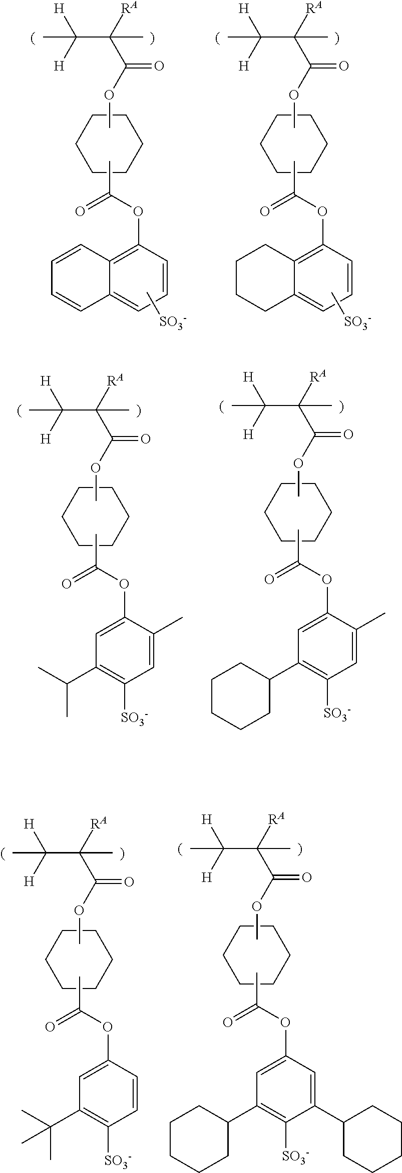

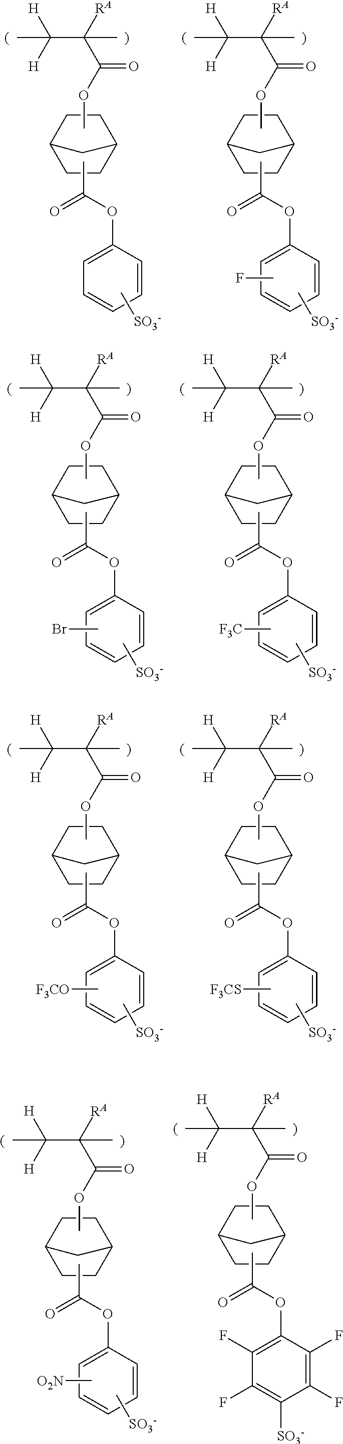

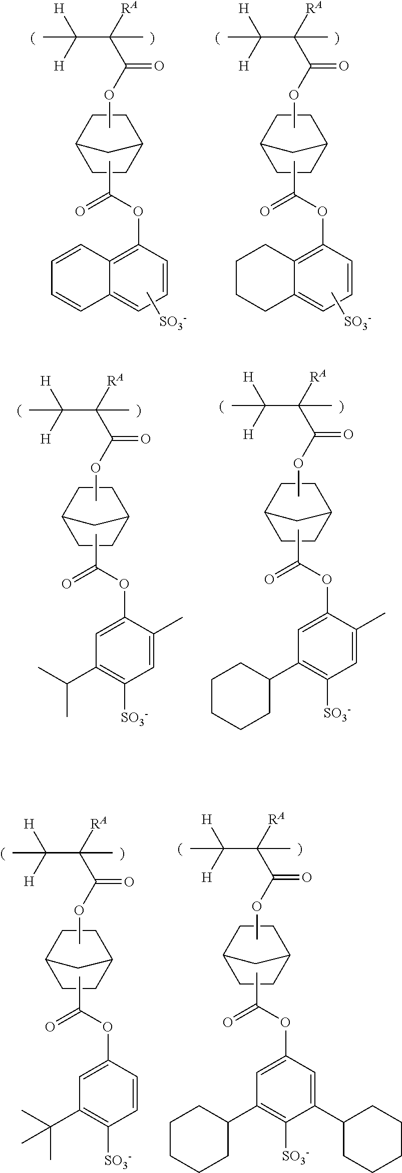

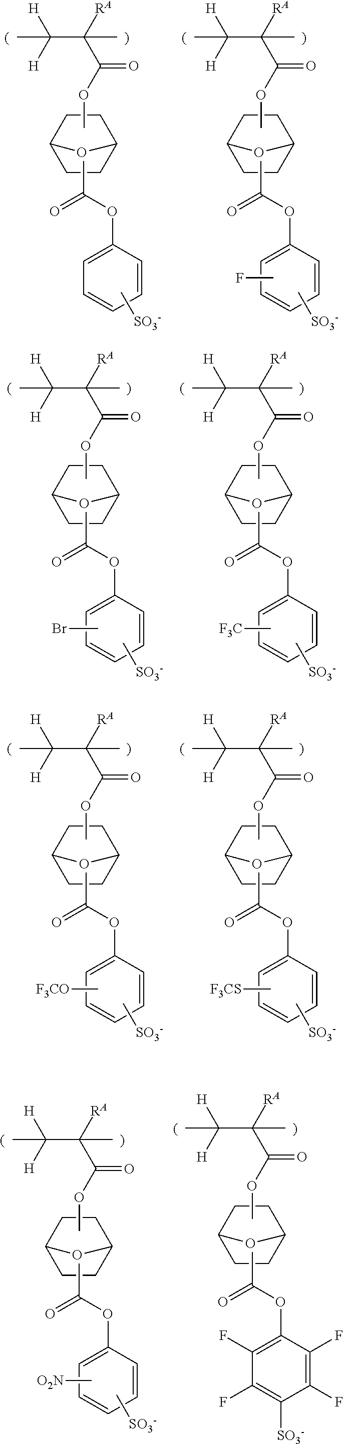

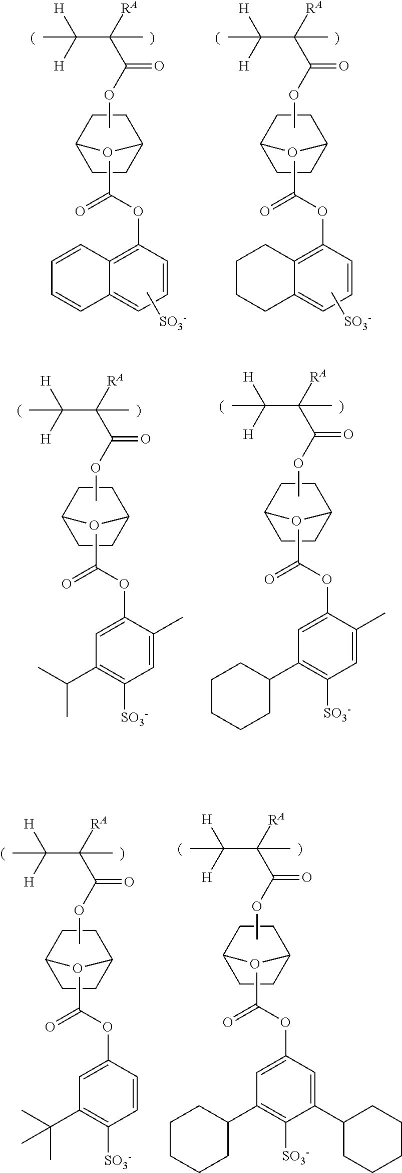

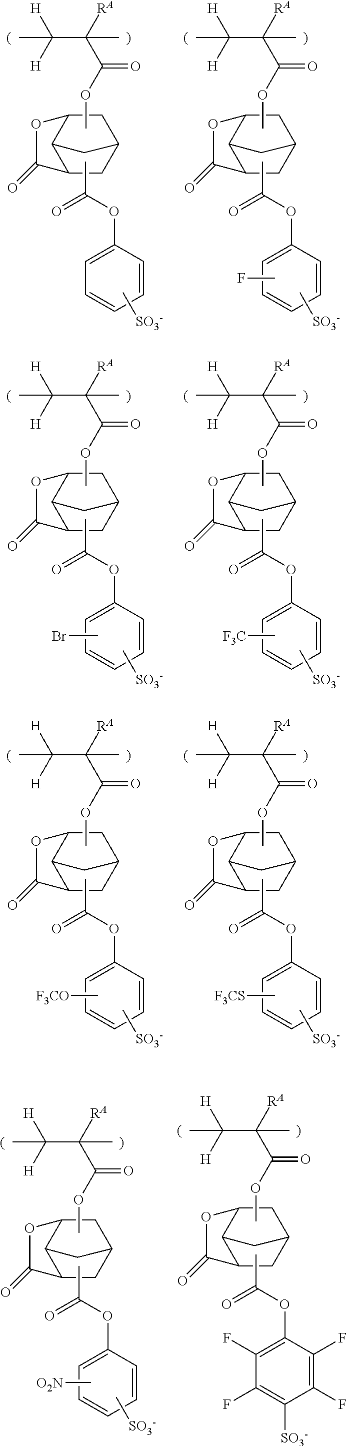

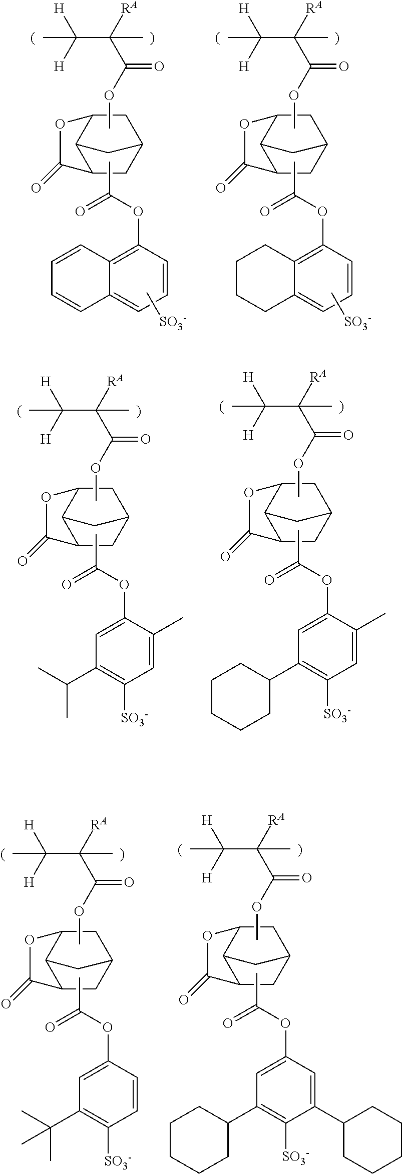

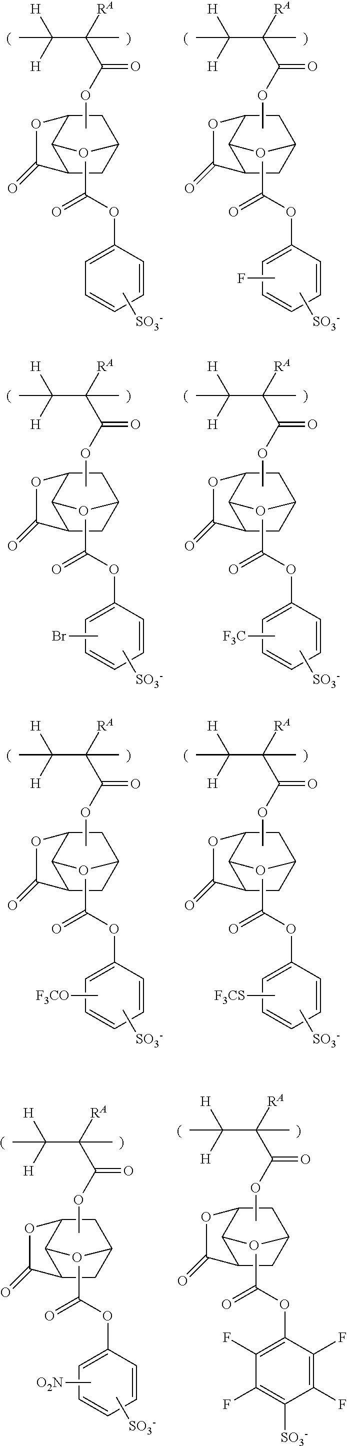

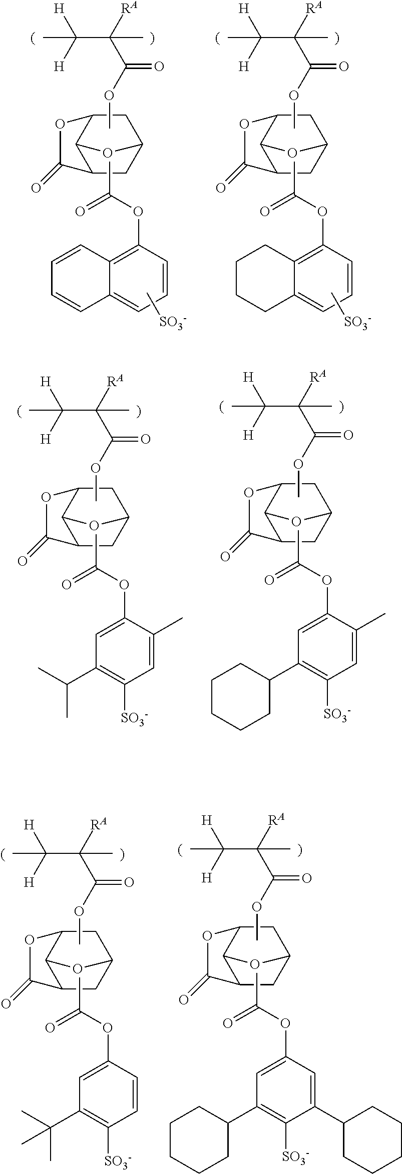

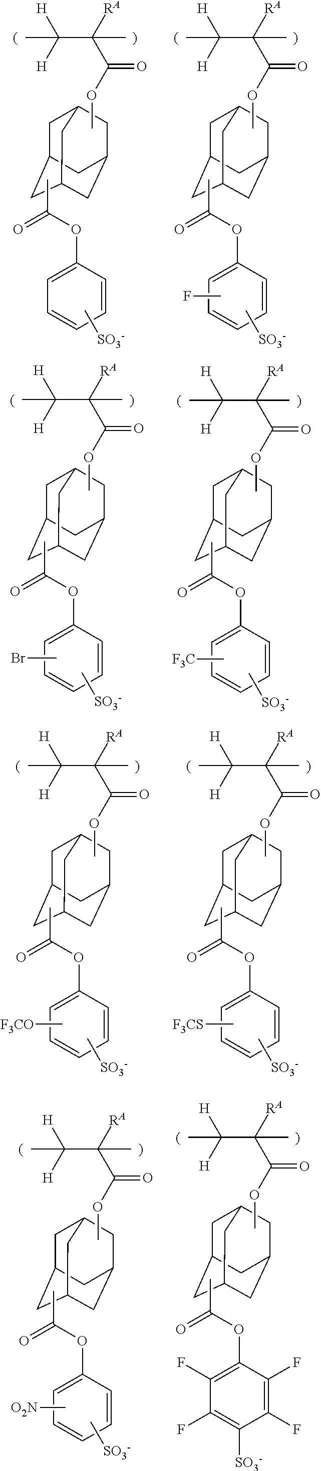

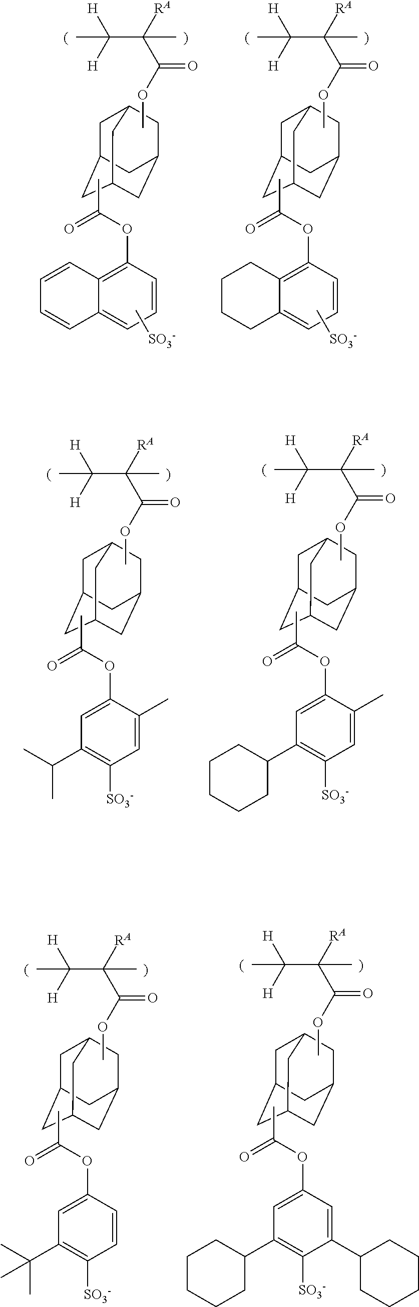

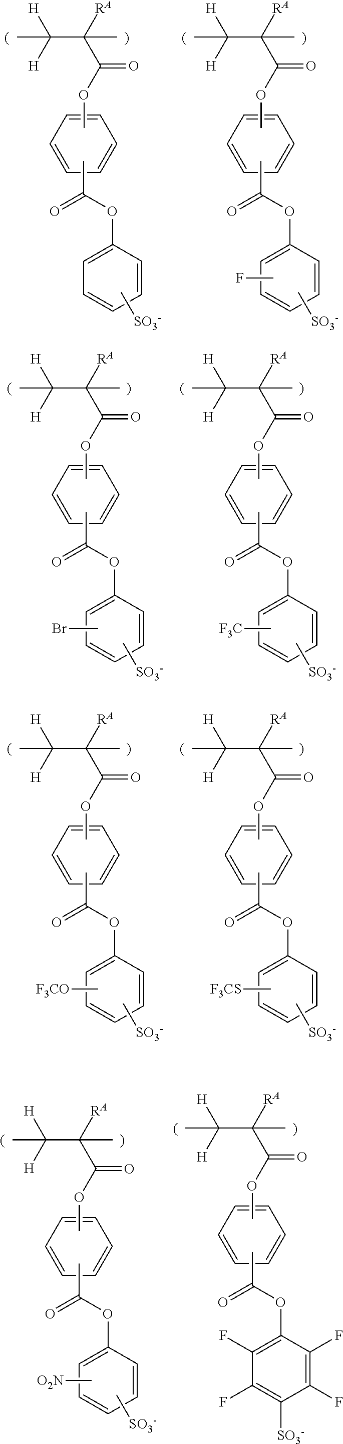

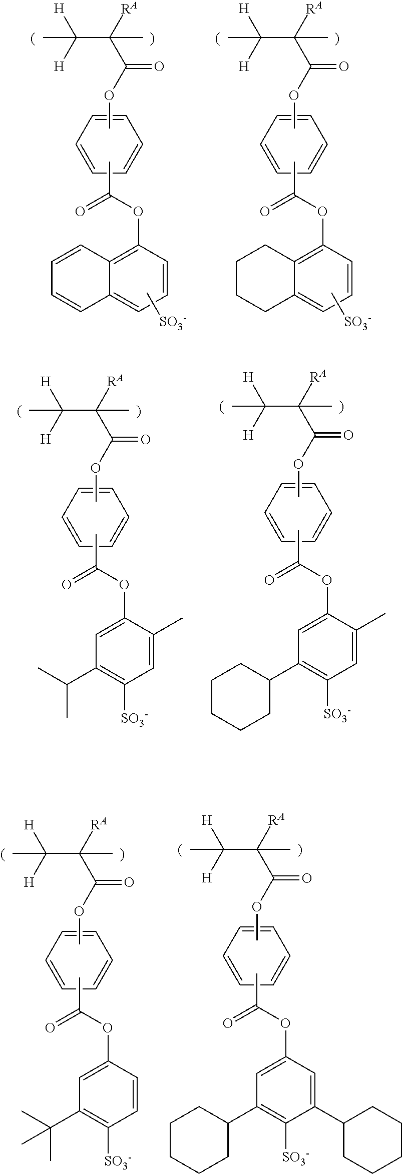

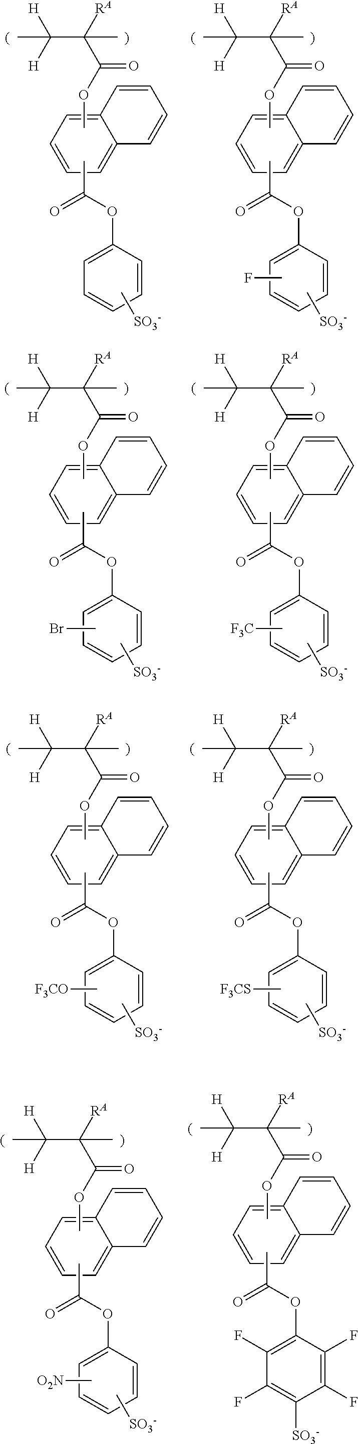

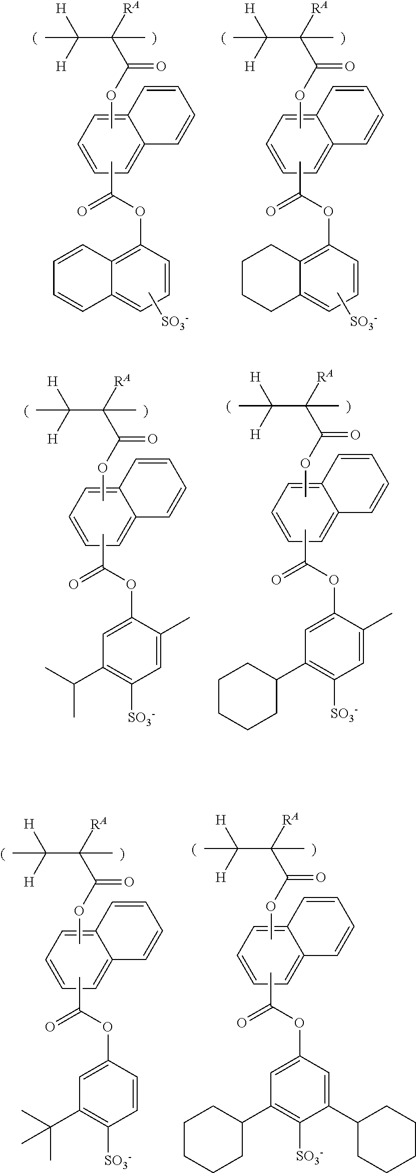

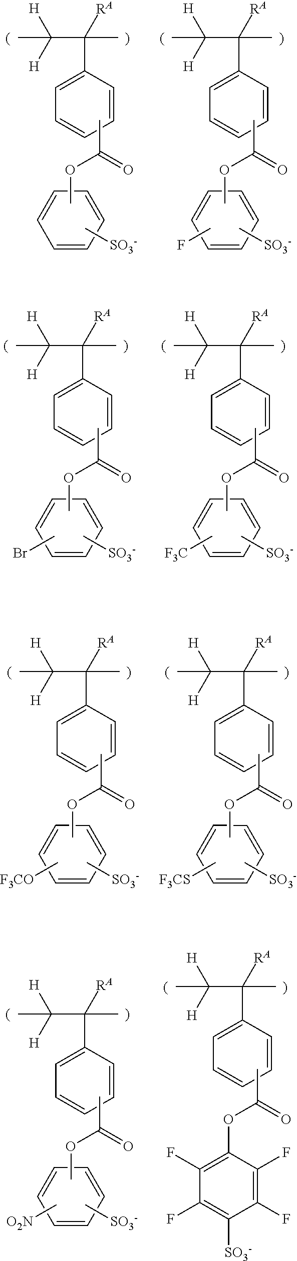

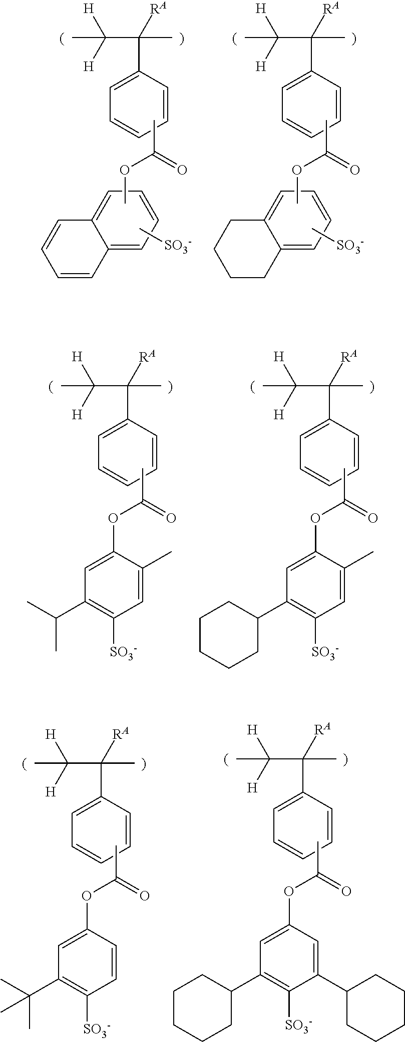

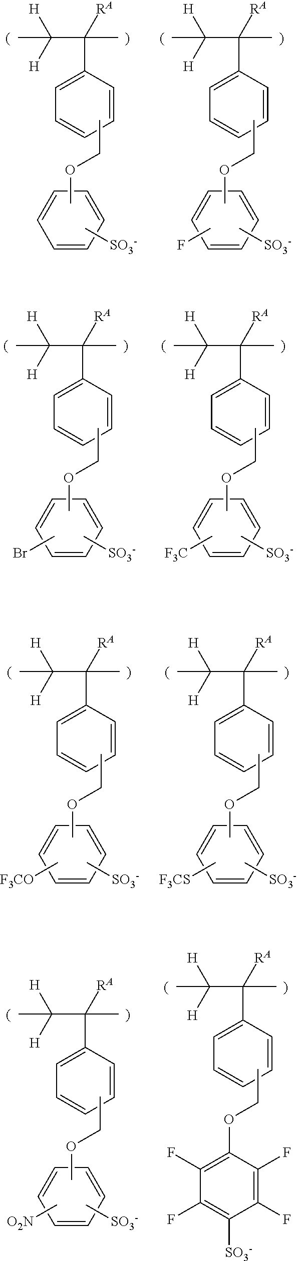

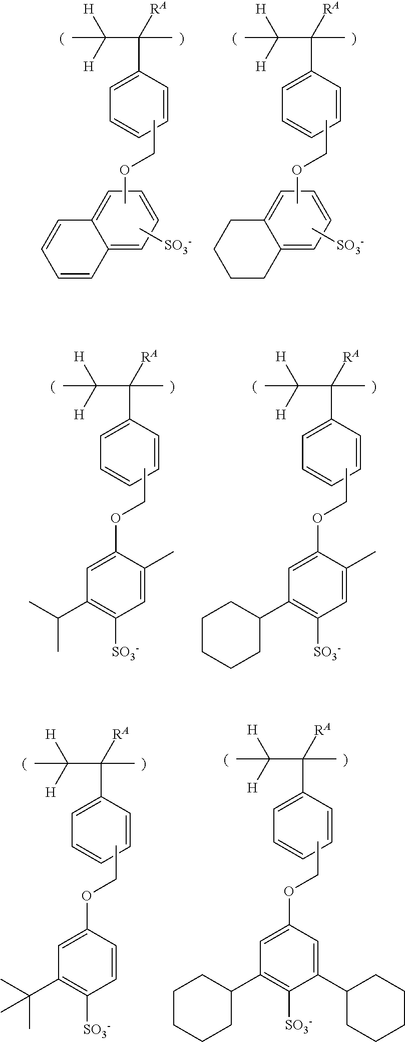

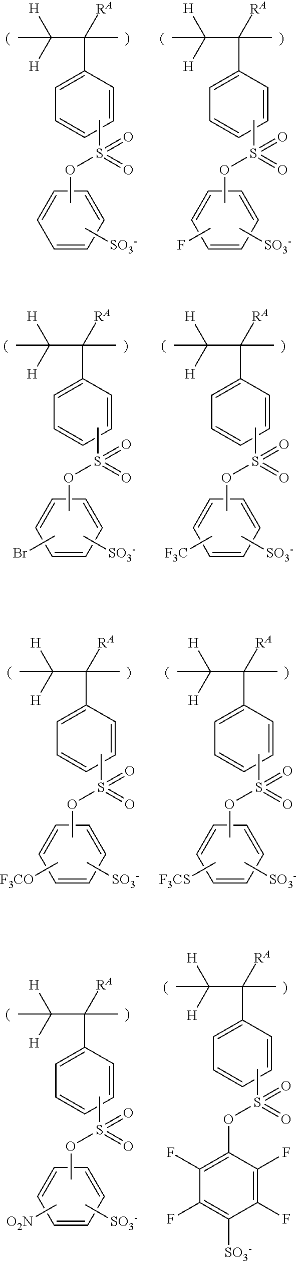

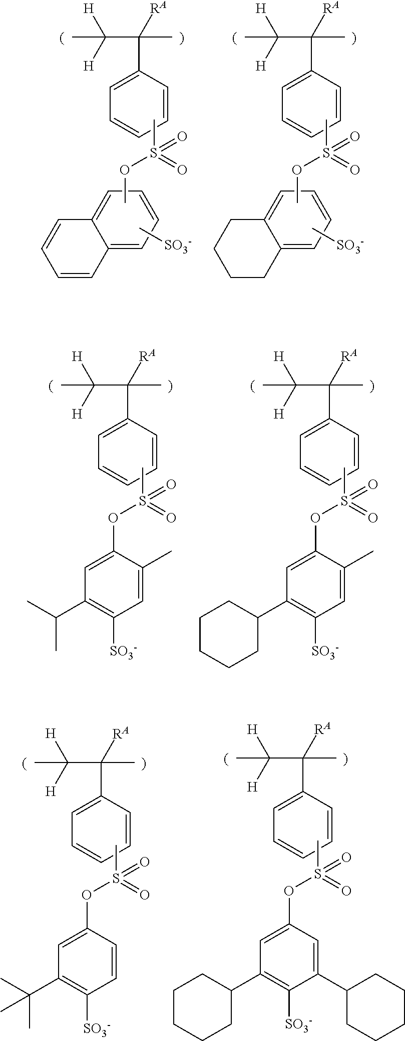

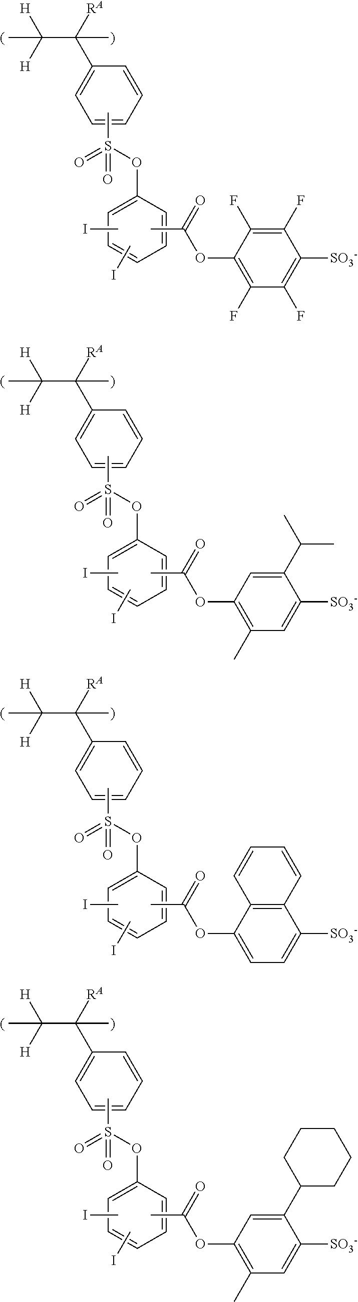

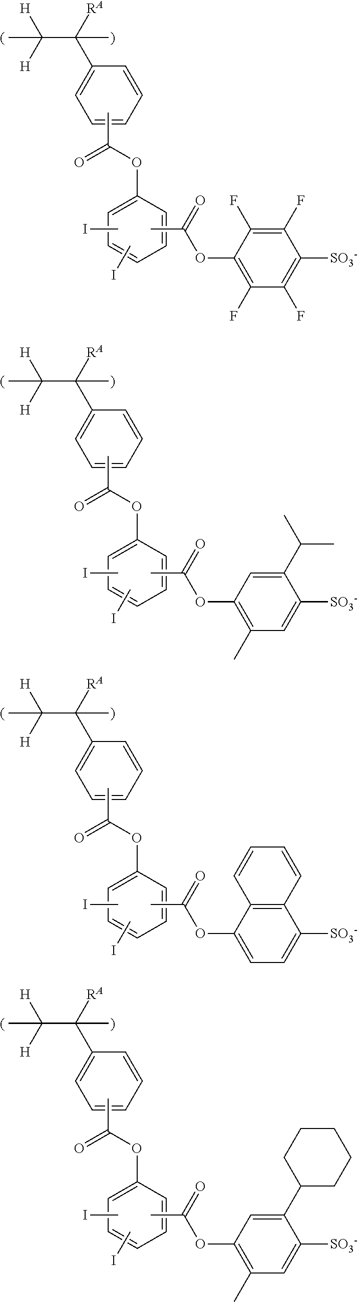

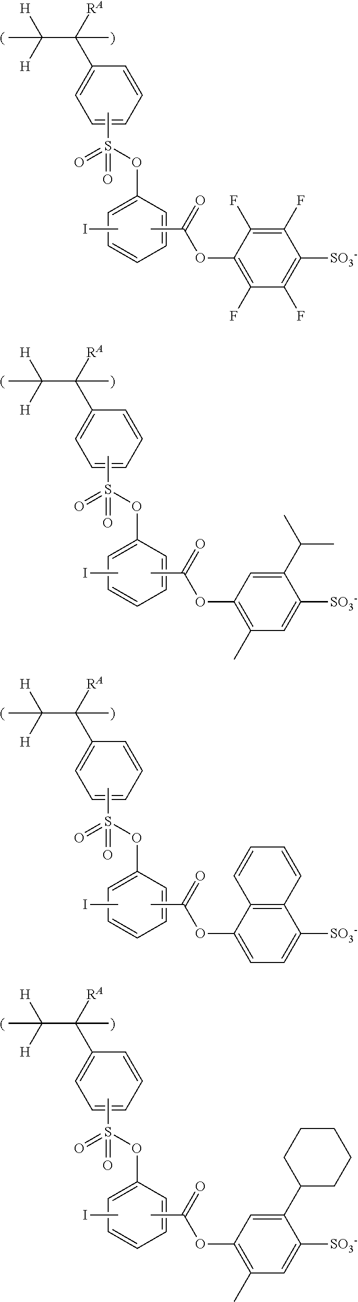

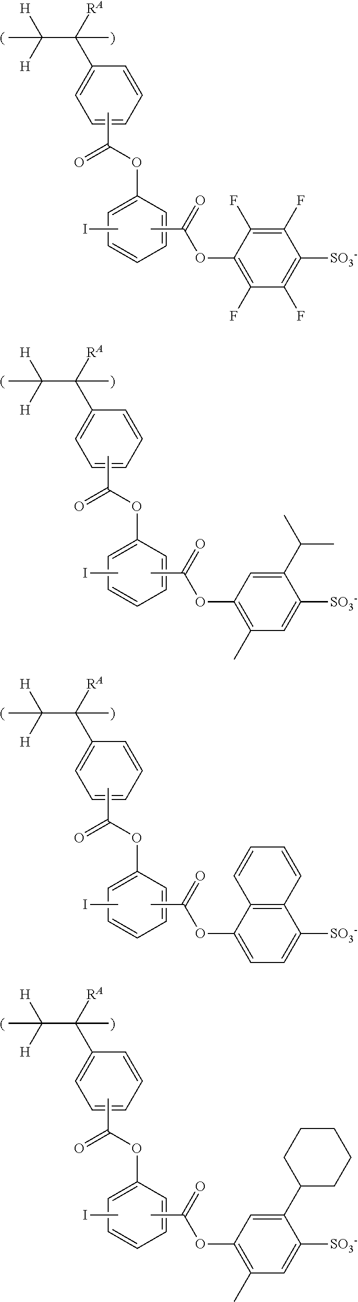

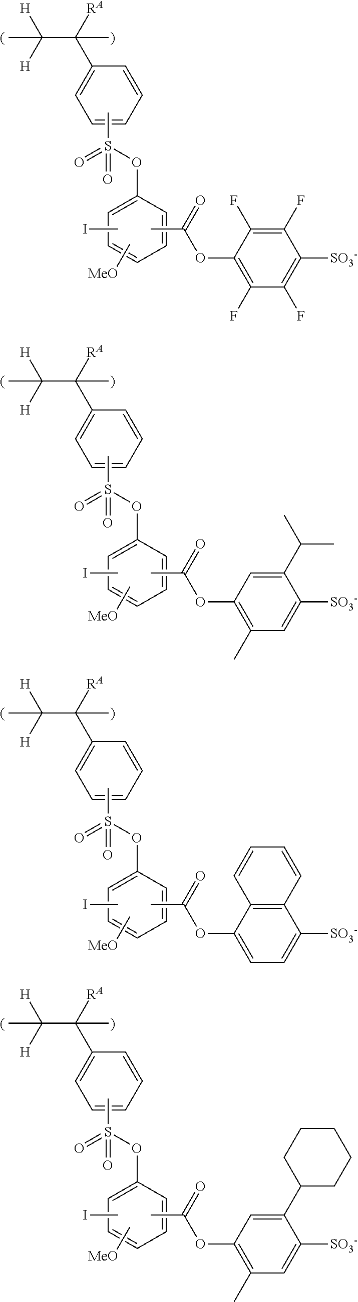

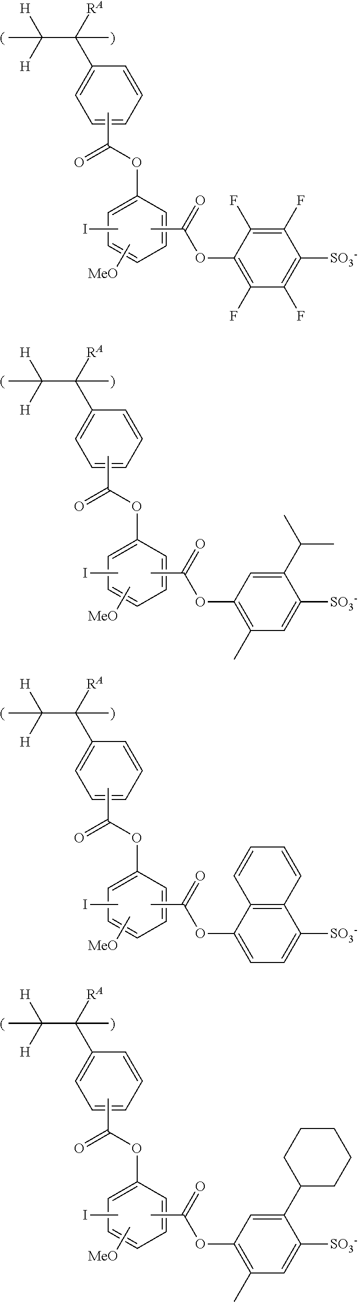

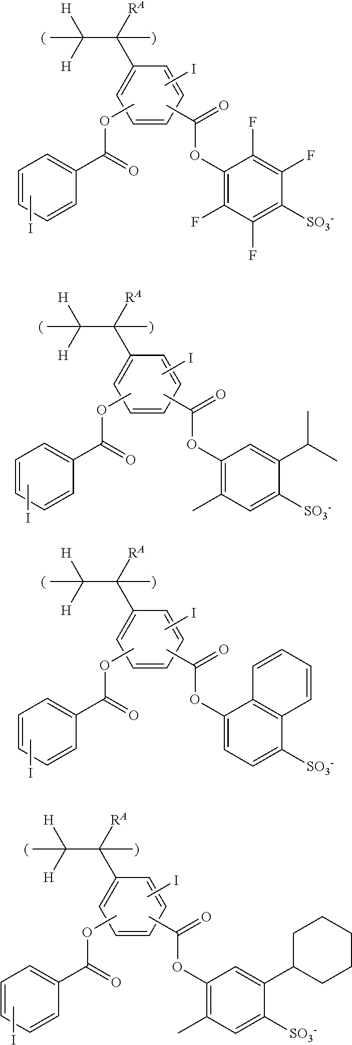

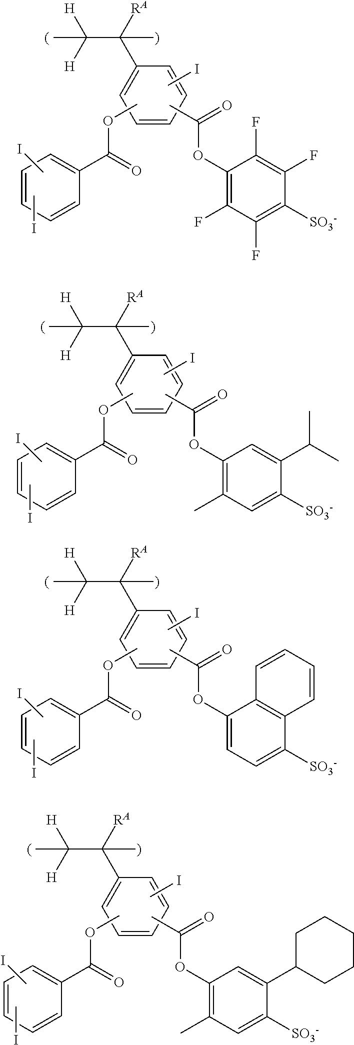

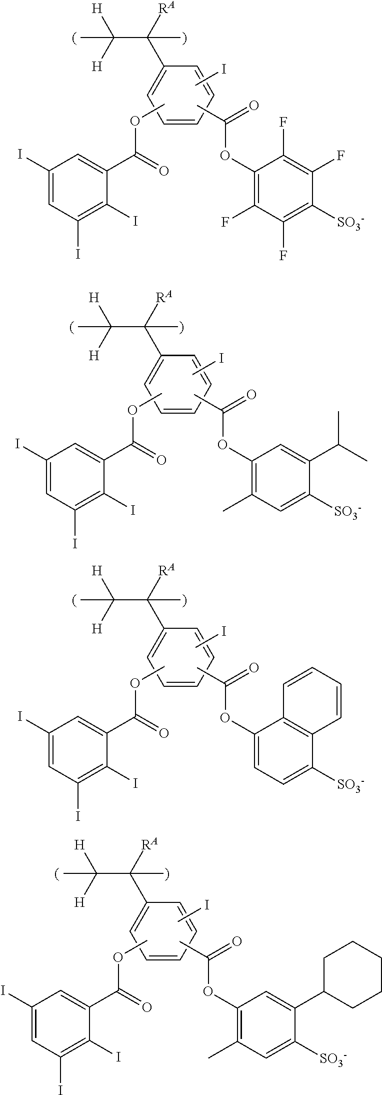

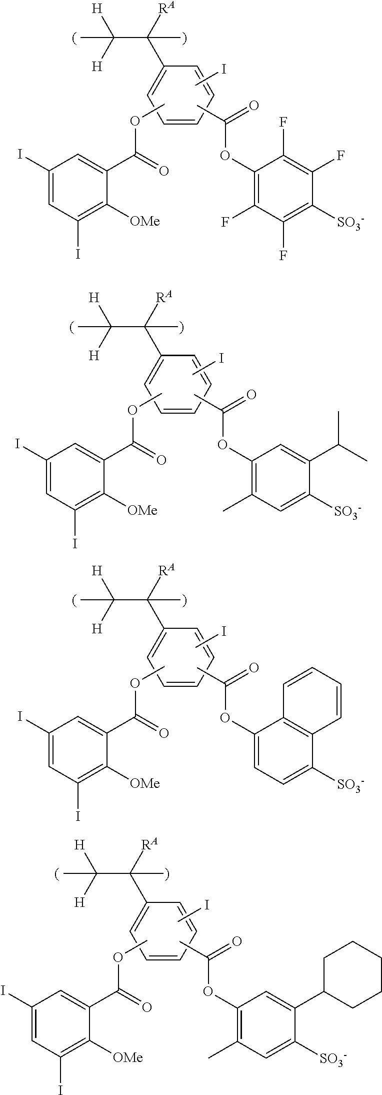

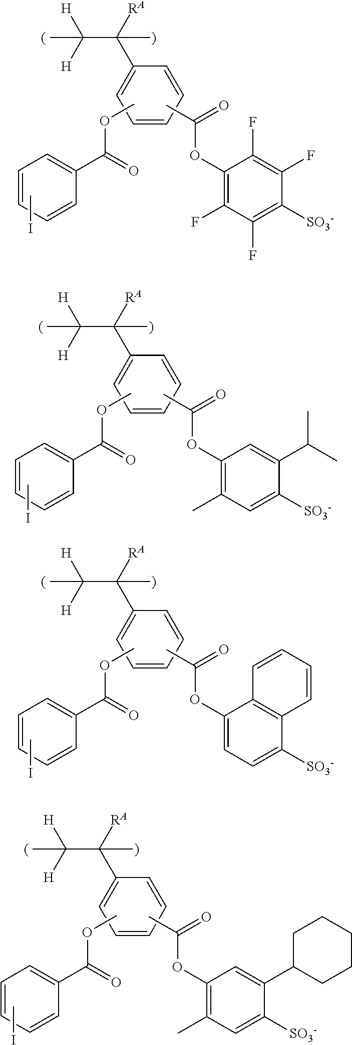

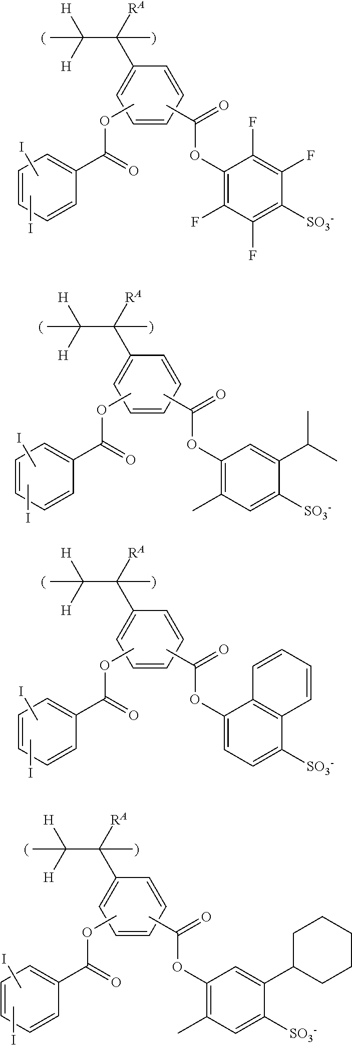

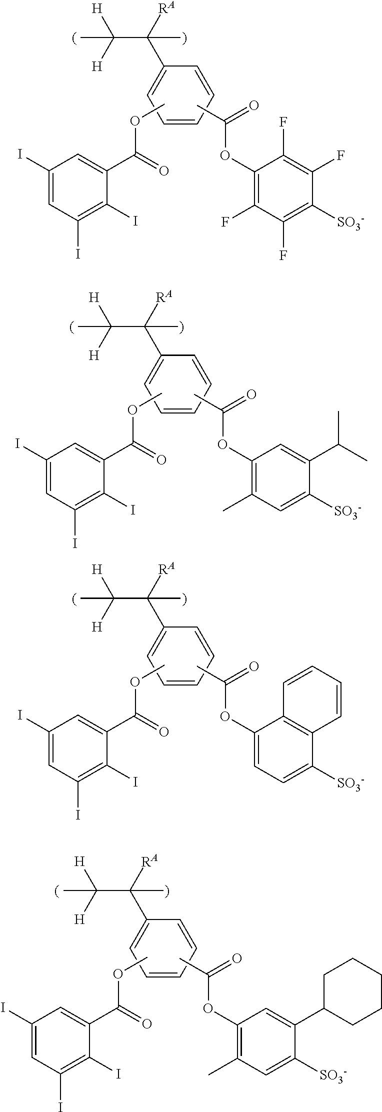

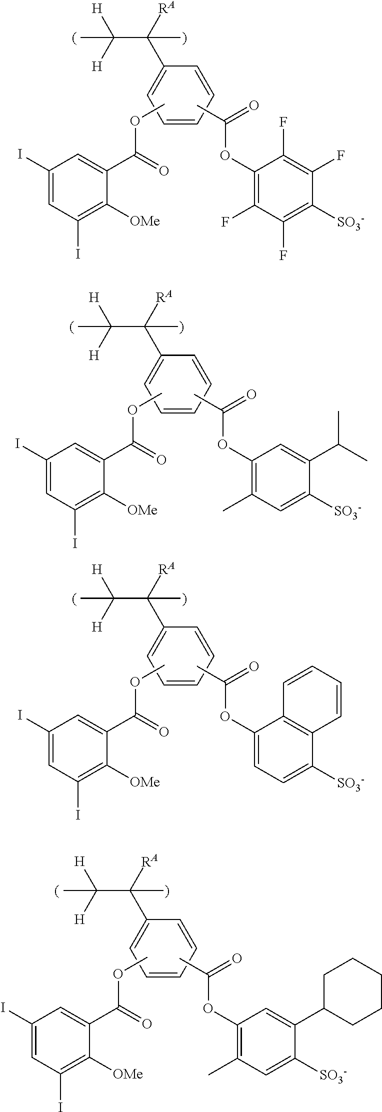

Specific examples of the anion of the repeat unit c4 include those described in paragraphs [0023] to [0026] of JP-A 2024-103464 and paragraphs [0419] and [0420] of JP-A 2024-120702.

Specific examples of the anion of the repeat unit c5 include, but are not limited to, those listed below. Note that, in the following formulas, R^{A} is as defined above.

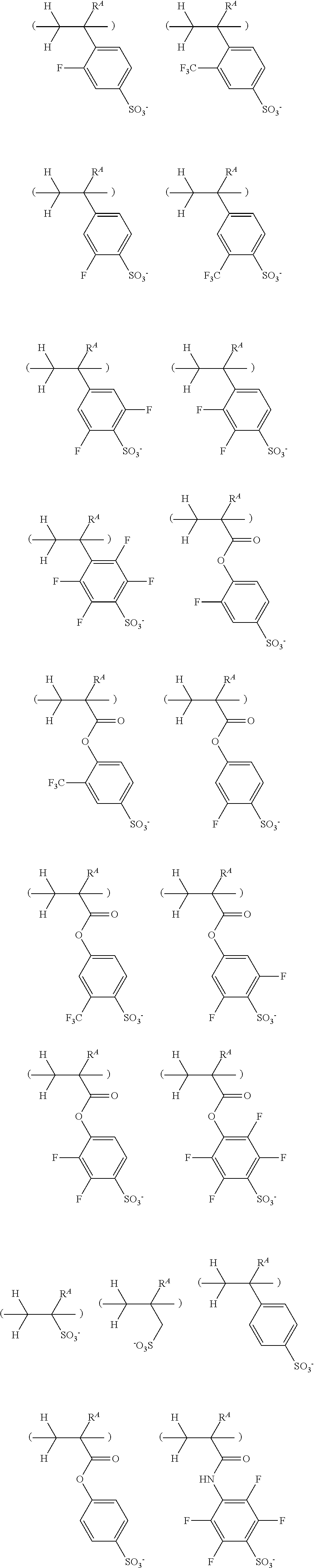

In the formulas (c2) to (c5), A⁺ is an onium cation. Examples of the onium cation include a sulfonium cation, an iodonium cation, and an ammonium cation, and a sulfonium cation and an iodonium cation are preferable. Specific examples of the sulfonium cation include those exemplified as specific examples of the cation of the sulfonium salt having the formula (1), those described in paragraphs [0102] to [0125] of JP-A 2024-3744, those described in paragraphs [0044] to [0049] of WO 2024/128017, and those described in paragraphs [0035] to [0046] of JP 7491173, but are not limited thereto.

As the sulfonium cation, a sulfonium cation having the following formula (sulfo-1) is also preferable.

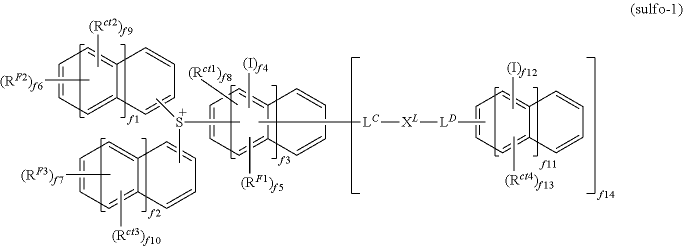

In the formula (sulfo-1), f1 is 0 or 1. When f1 is 0, it is a benzene ring, and when f1 is 1, it is a naphthalene ring, but a benzene ring in which f1 is 0 is preferable from a viewpoint of solvent solubility. f2 is 0 or 1. When f2 is 0, it is a benzene ring, and when f2 is 1, it is a naphthalene ring, but a benzene ring in which f2 is 0 is preferable from a viewpoint of solvent solubility. f3 is 0 or 1. When f3 is 0, it is a benzene ring, and when f3 is 1, it is a naphthalene ring, but a benzene ring in which f3 is 0 is preferable from a viewpoint of solvent solubility.

In the formula (sulfo-1), f4 is 0, 1, 2, 3, or 4. The larger the number of iodine atoms in the cation structure, the higher absorption particularly for EUV, but since there is a concern that solvent solubility is poor and precipitation occurs in a resist composition, f4 is preferably 0, 1, 2, or 3, and more preferably 0, 1, or 2.

In the formula (sulfo-1), f5 is 0, 1, 2, 3, or 4. f5 is preferably 0, 1, 2, or 3, and more preferably 0, 1, or 2 from a viewpoint of raw material procurement. f6 is 0, 1, 2, 3, 4, 5, or 6. f6 is preferably 0, 1, 2, or 3, and more preferably 0, 1, or 2 from a viewpoint of raw material procurement. f7 is 0, 1, 2, 3, 4, 5, or 6. f7 is preferably 0, 1, 2, or 3, and more preferably 0, 1, or 2 from a viewpoint of raw material procurement.

In the formula (sulfo-1), f8 is 0, 1, or 2. f8 is preferably 0 or 1 from a viewpoint of raw material procurement. f9 is 0, 1, or 2. f9 is preferably 0 or 1 from a viewpoint of raw material procurement. f10 is 0, 1, or 2. f10 is preferably 0 or 1 from a viewpoint of raw material procurement.

In the formula (sulfo-1), f11 is 0 or 1. When f11 is 0, it is a benzene ring, and when f11 is 1, it is a naphthalene ring, but a benzene ring in which f11 is 0 is preferable from a viewpoint of solvent solubility.

In the formula (sulfo-1), f12 is 0, 1, 2, 3, or 4. The larger the number of iodine atoms in the cation structure, the higher absorption particularly for EUV, but since there is a concern that solvent solubility is poor and precipitation occurs in a resist composition, f12 is preferably 0, 1, 2, or 3, and more preferably 0, 1, or 2.

In the formula (sulfo-1), f13 is 0, 1, or 2. f13 is preferably 0 or 1 from a viewpoint of raw material procurement. f14 is 0, 1, or 2. f14 is preferably 0 or 1 from a viewpoint of synthesis.

Provided that 0≤f6+f9≤4 when f1 is 0, and 0≤f6+f9≤6 when f1 is 1. 0≤f7+f10≤4 when f2 is 0, and 0≤f7+f10≤6 when f2 is 1. 1≤f4+f5+f8+f14≤4 when f3 is 0, and 1≤f4+f5+f8+f14≤6 when f3 is 1. 0≤f12+f13≤4 when f11 is 0, and 0≤f12+f13≤6 when f11 is 1. f4+f12≥1.

In the formula (sulfo-1), R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group. Among these, R^{F1} to R^{F3} are each preferably a trifluoromethyl group, a trifluoromethoxy group, or a trifluorothiomethoxy group. When f5 is 2, 3, or 4, R^{F1}s may be the same as or different from each other. When f6 is 2, 3, 4, 5, or 6, R^{F2}s may be the same as or different from each other. When f7 is 2, 3, 4, 5, or 6, R^{F3}s may be the same as or different from each other.

In the formula (sulfo-1), each of R^{ct1} to R^{ct4} is a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and hydrocarbyl moieties of the hydrocarbyloxy group and the hydrocarbylthio group may be saturated or unsaturated, and may be linear, branched, or cyclic. Specific examples thereof include those similar to those exemplified as the hydrocarbyl group of R¹ and R² in the description of the formula (1). Some or all of the hydrogen atoms of the hydrocarbyl group and the hydrocarbyl moieties of the hydrocarbyloxy group and the hydrocarbylthio group may be replaced with a group containing a heteroatom such as an oxygen atom, a sulfur atom, a nitrogen atom, or a halogen atom, some of the (—CH₂-)s in the hydrocarbyl group may be replaced with a group containing a heteroatom such as an oxygen atom, a sulfur atom, or a nitrogen atom, and as a result, the hydrocarbyl group and the hydrocarbyl moieties of the hydrocarbyloxy group and the hydrocarbylthio group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

When f8 is 2, two R^{ct1}s may be the same as or different from each other, and the two R^{ct1}s may be bonded to each other to form a ring together with carbon atoms to which they are bonded. When f9 is 2, two R²s may be the same as or different from each other, and the two R^{ct2}s may be bonded to each other to form a ring together with carbon atoms to which they are bonded. When f10 is 2, two R^{ct3}s may be the same as or different from each other, and the two R^{ct3}s may be bonded to each other to form a ring together with carbon atoms to which they are bonded. When f13 is 2, two R^{ct4}s may be the same as or different from each other, and the two R^{ct4}s may be bonded to each other to form a ring together with carbon atoms to which they are bonded. Specific examples of the ring formed at this time include a cyclopropane ring, a cyclobutane ring, a cyclopentane ring, a cyclohexane ring, a norbornane ring, and an adamantane ring. Some or all of the hydrogen atoms of the ring may be replaced with a group containing a heteroatom such as an oxygen atom, a sulfur atom, a nitrogen atom, or a halogen atom, some of the (—CH₂-)s in the ring may be replaced with a group containing a heteroatom such as an oxygen atom, a sulfur atom, or a nitrogen atom, and as a result, the ring may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

Aromatic rings directly bonded to S⁺ in the sulfonium cation having the formula (sulfo-1) may be bonded to each other to form a ring together with S⁺. At this time, specific examples of the structure of the ring include those having the following formulas:

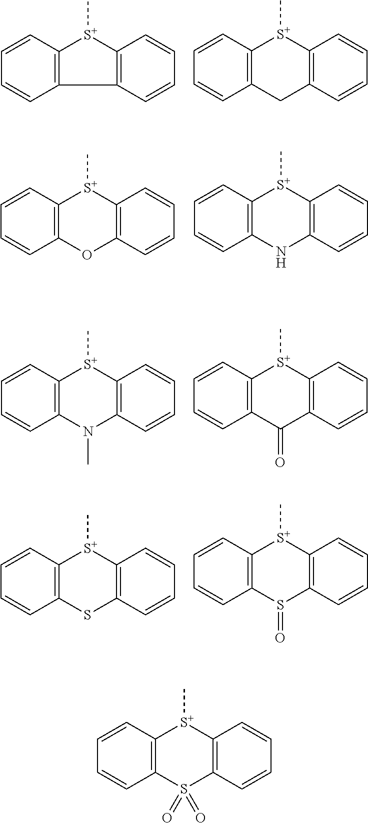

wherein the dashed line is a bond.

In the formula (sulfo-1), L^{C} and L^{D} are each independently a single bond, an ether bond, an ester bond, a sulfonate bond, an amide bond, a sulfonic acid amide bond, a carbonate bond, or a carbamate bond. Among these, L^{C} is preferably a single bond, an ether bond, an ester bond, or a sulfonate bond, and more preferably an ester bond or a sulfonate bond. L^{D} is preferably a single bond, an ether bond, or an ester bond, and more preferably a single bond.

In the formula (sulfo-1), X^{L} is a single bond or a C₁-C₄₀ hydrocarbylene group which may contain a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom.

Specific examples of the C₁-C₄₀ hydrocarbylene group of X^{L} which may contain a heteroatom include, but are not limited to, those listed below. Note that, in the following formulas, *s are a bond with L^{E} and a bond with L^{F}, respectively

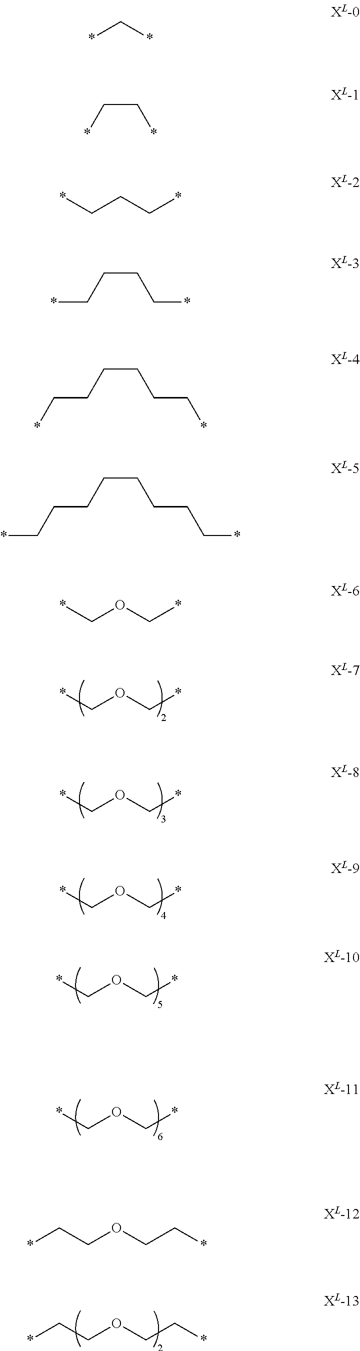

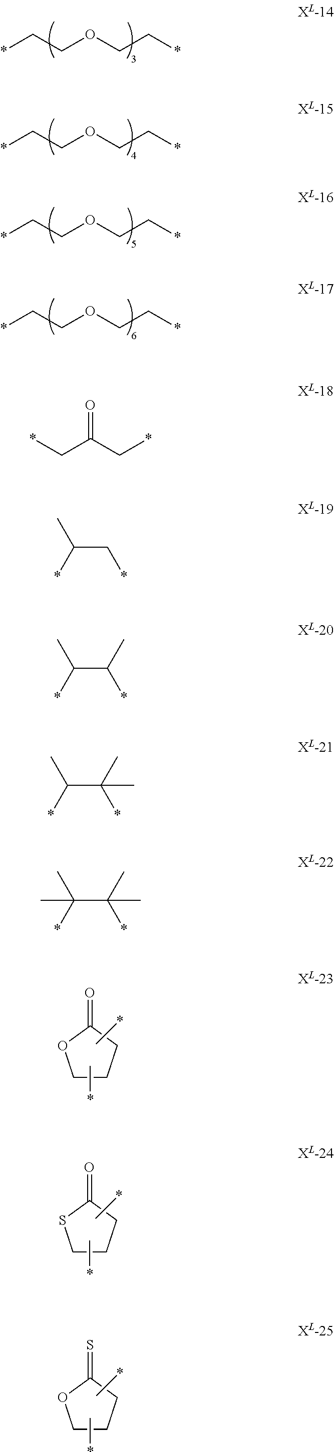

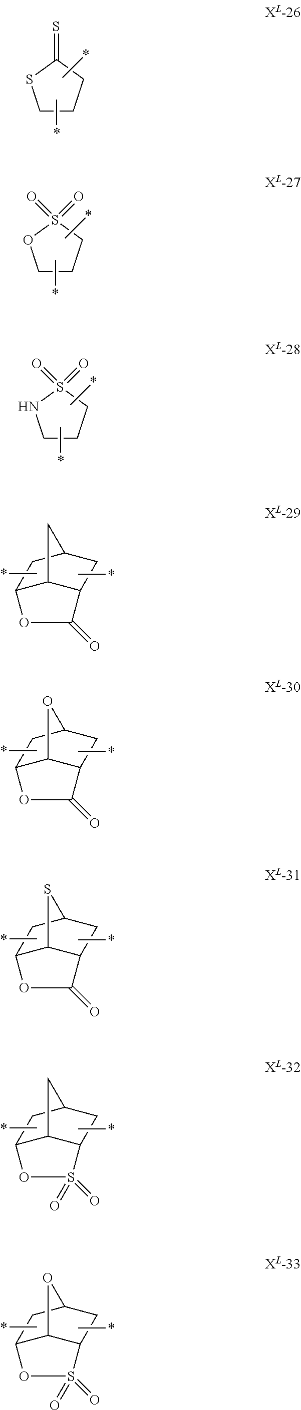

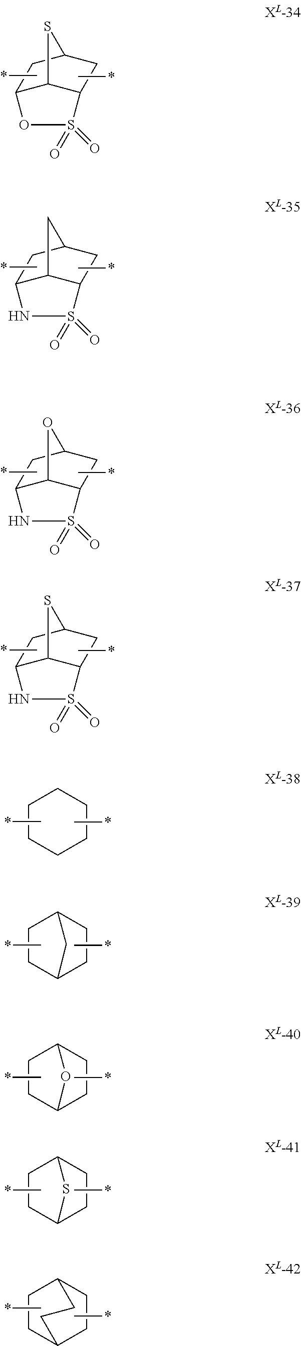

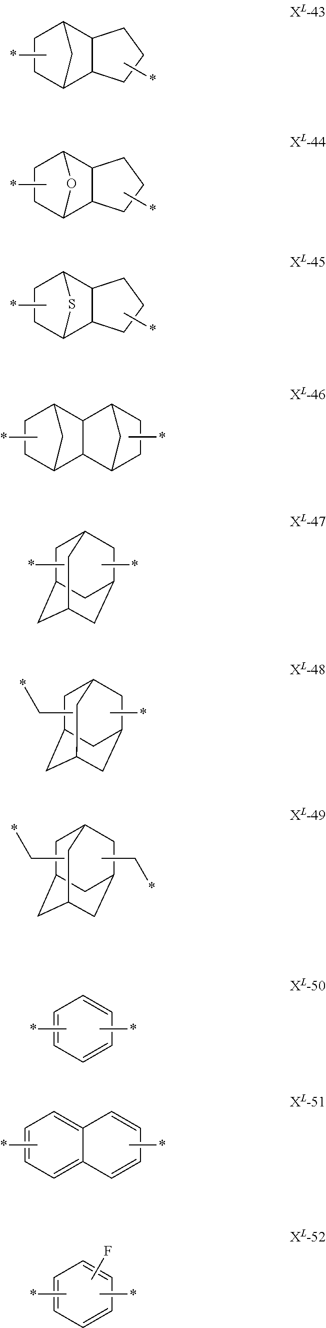

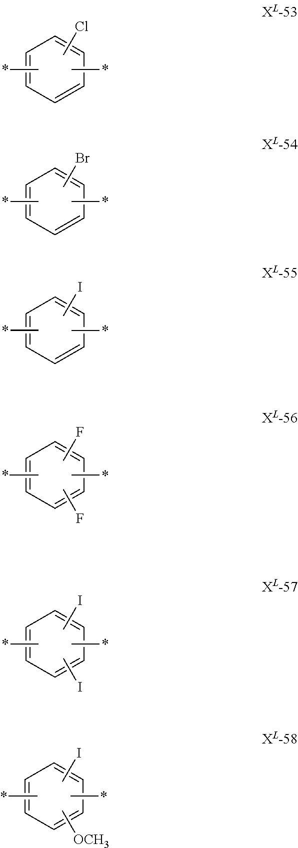

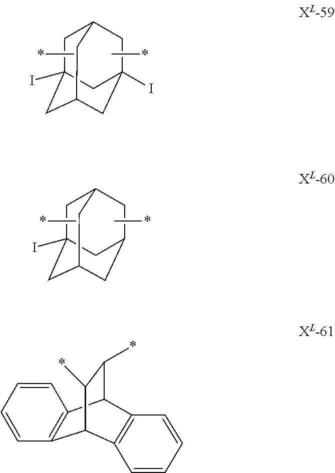

Among these, X^{L}-0 to X^{L}-22, X^{L}-29 to X^{L}-34, and X^{L}-47 to X^{L}-61 are preferable.

The sulfonium cation having the formula (sulfo-1) preferably has the following formula (sulfo-1-1):

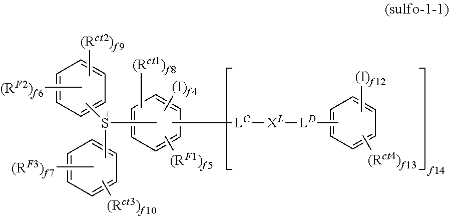

wherein f4 to f10, f12 to f14, R^{F1} to R^{F3}, R^{ct1} to R^{ct4}, L^{C}, L^{D}, and X^{L} are as defined above.

The sulfonium cation having the formula (sulfo-1-1) preferably has the following formula (sulfo-1-2):

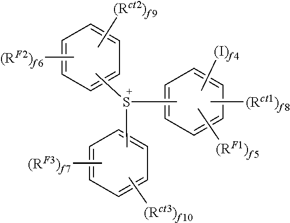

wherein f4 to f10, R^{F1} to R^{F3}, and R^{ct1} to R^{ct3} are as defined above.

Specific examples of the sulfonium cation having the formula (sulfo-1) include, but are not limited to, those listed below. Note that, in the following formulas, Me is a methyl group.

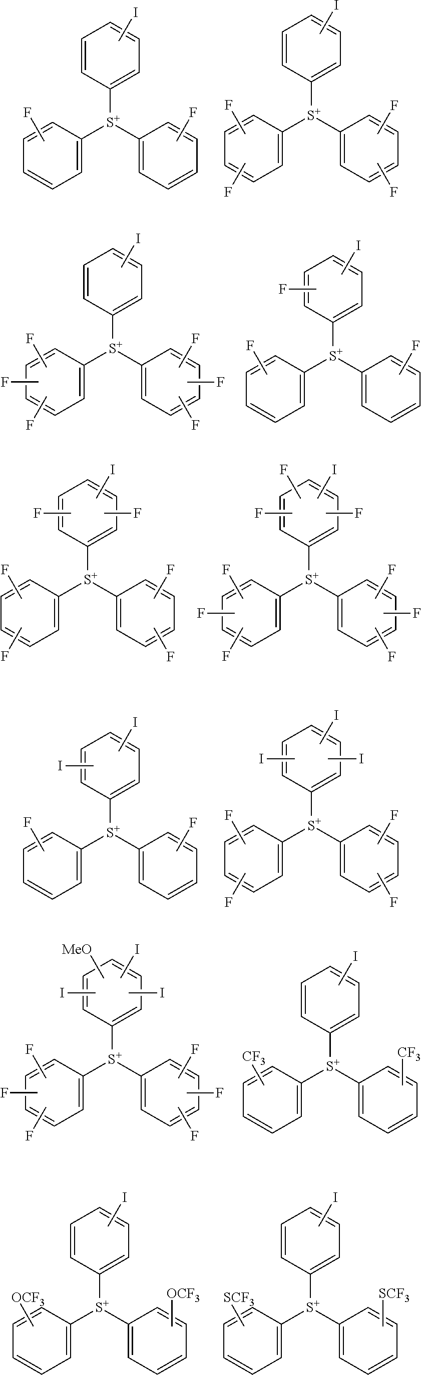

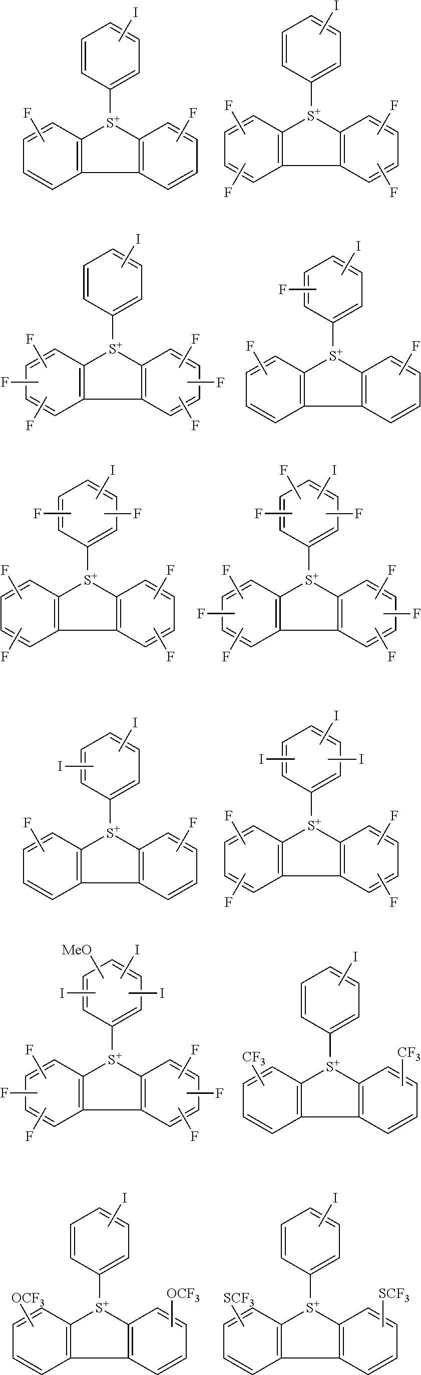

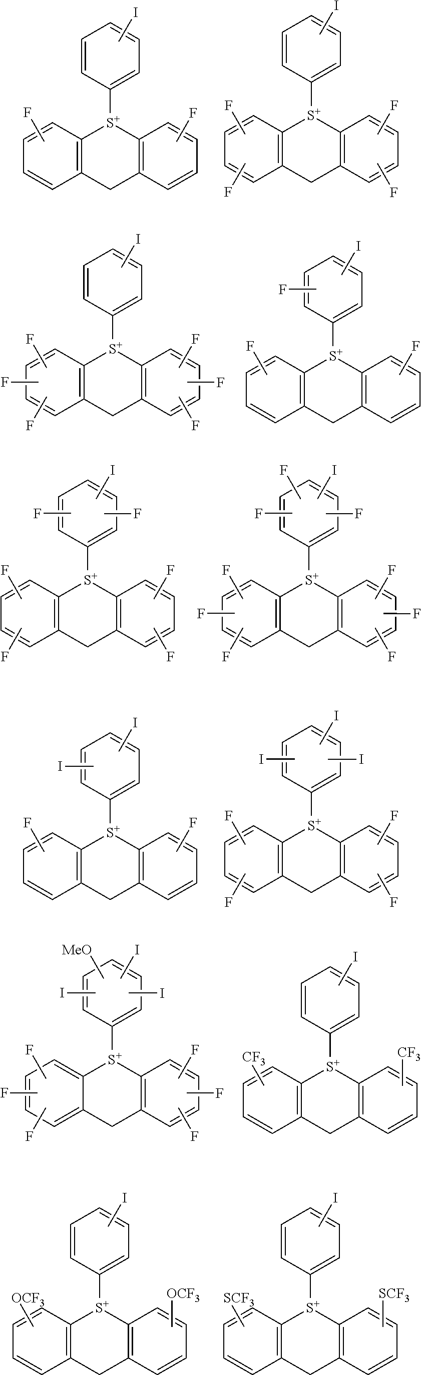

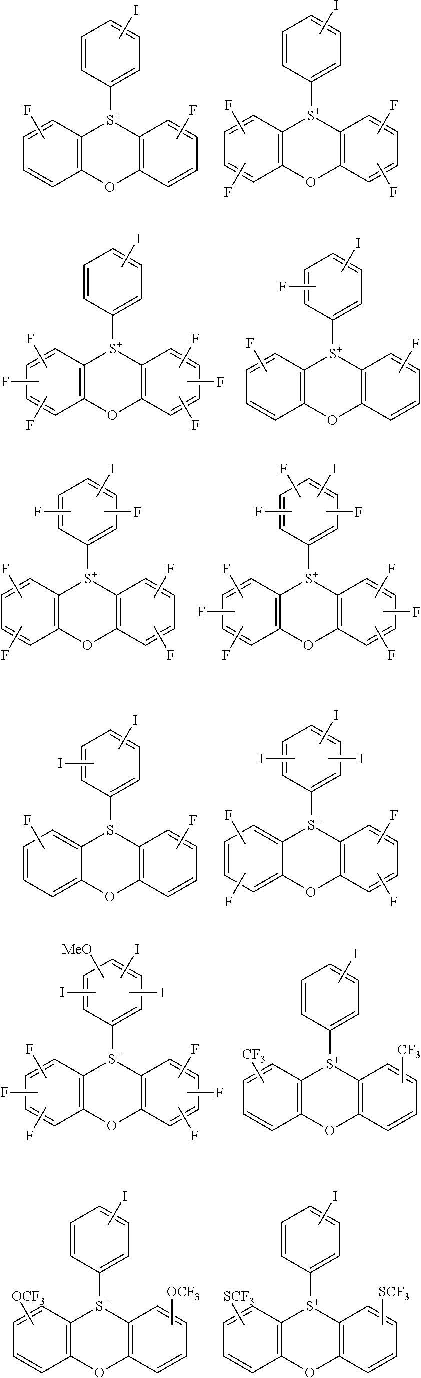

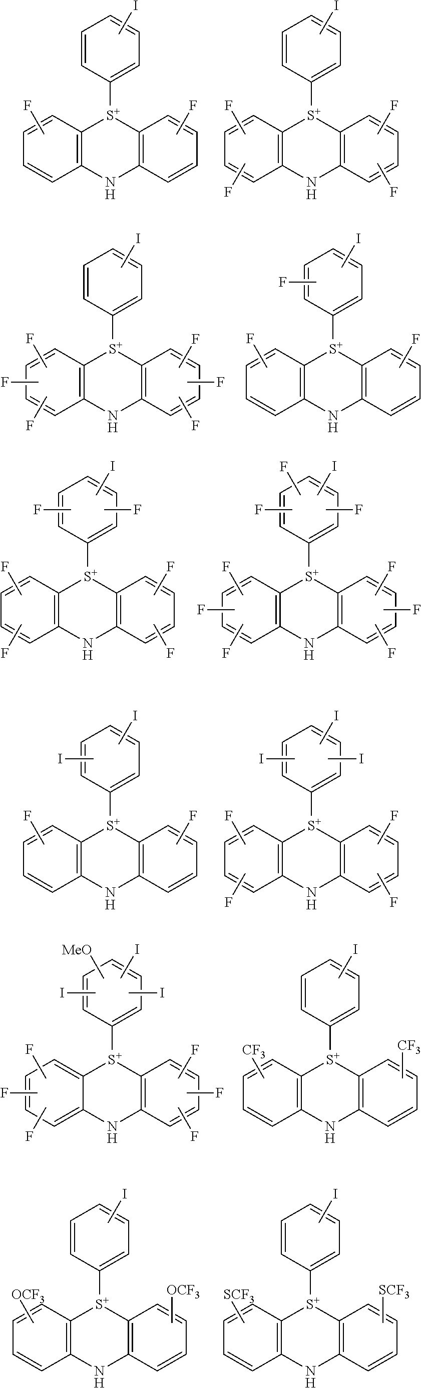

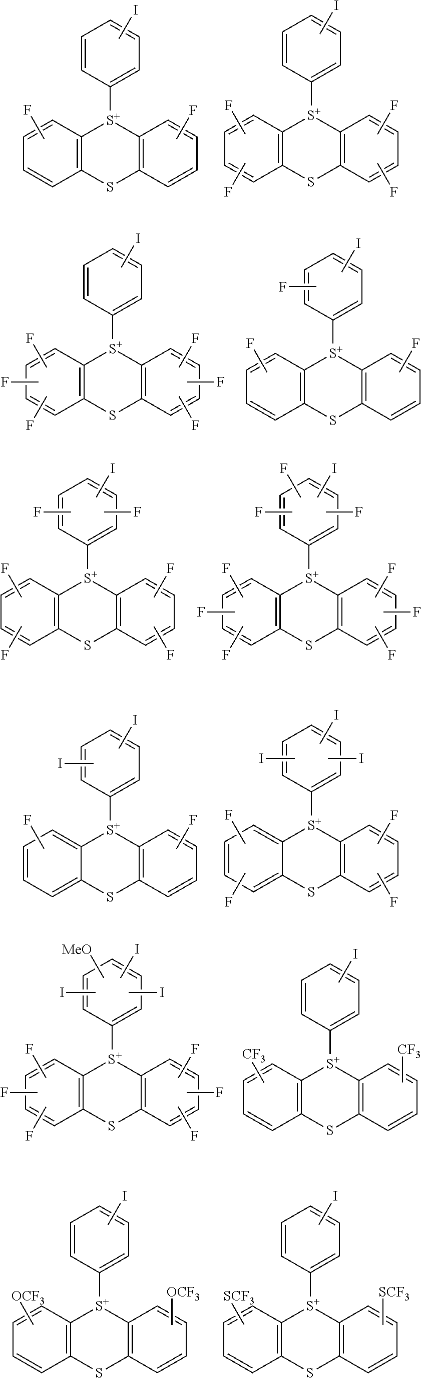

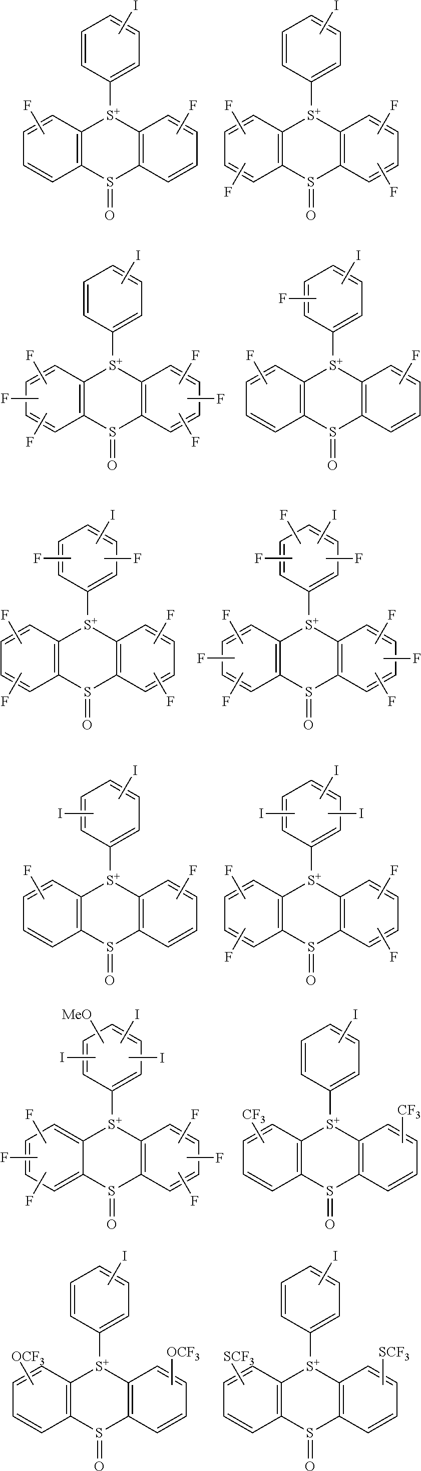

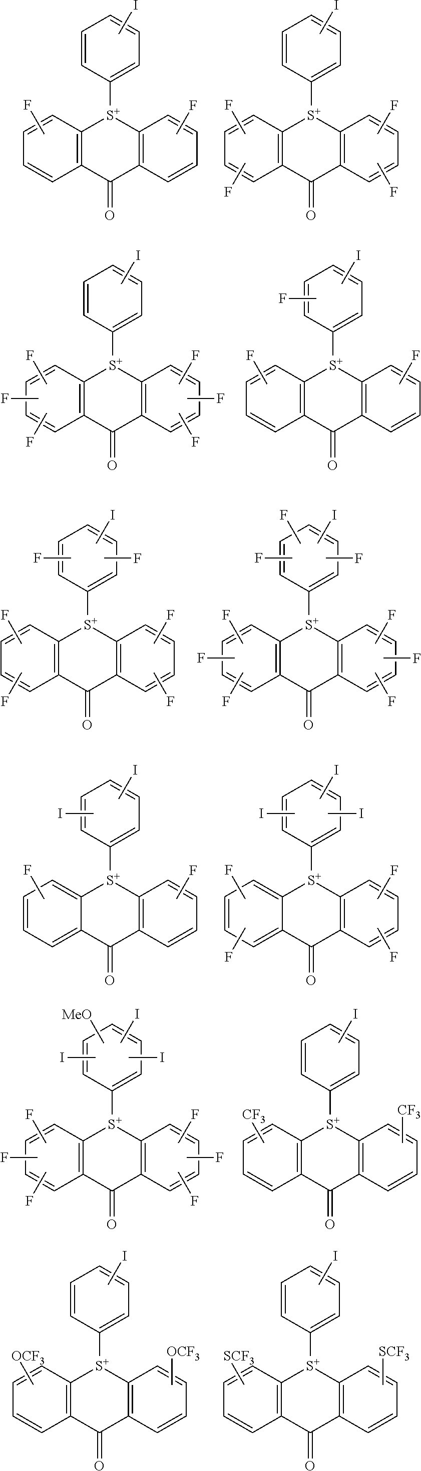

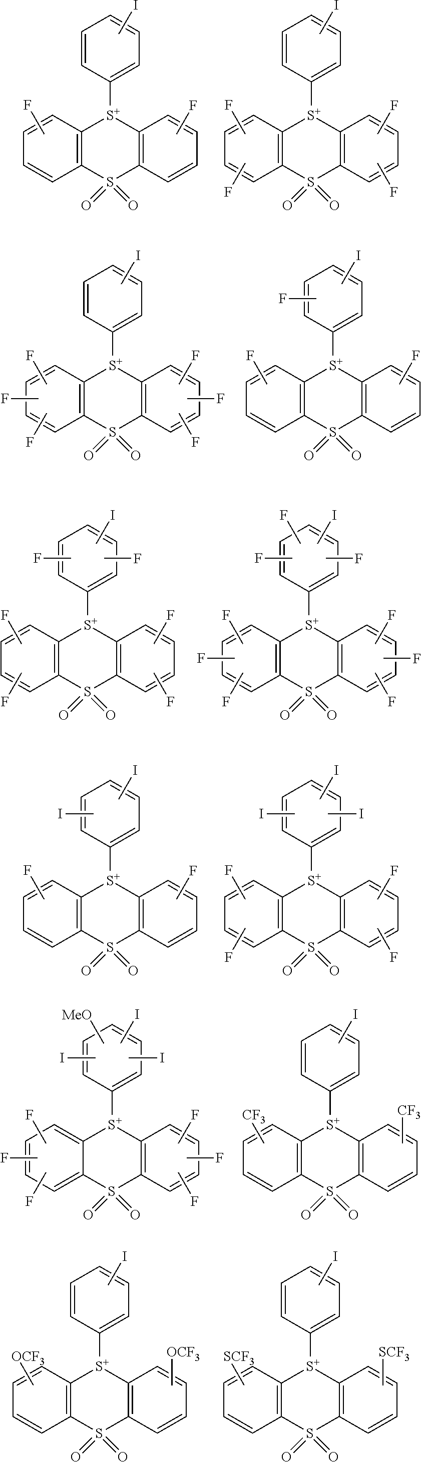

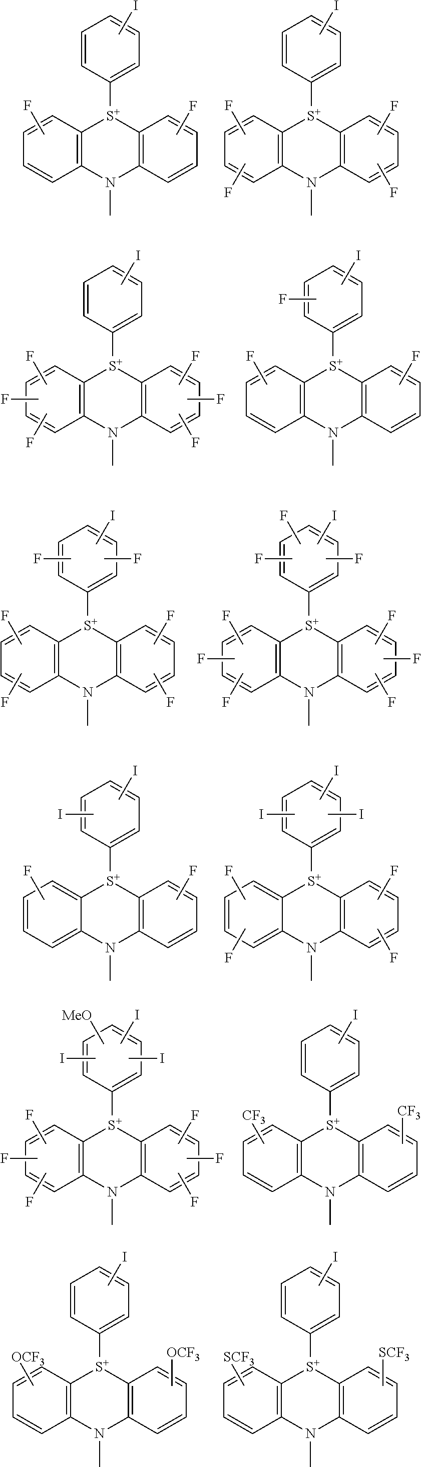

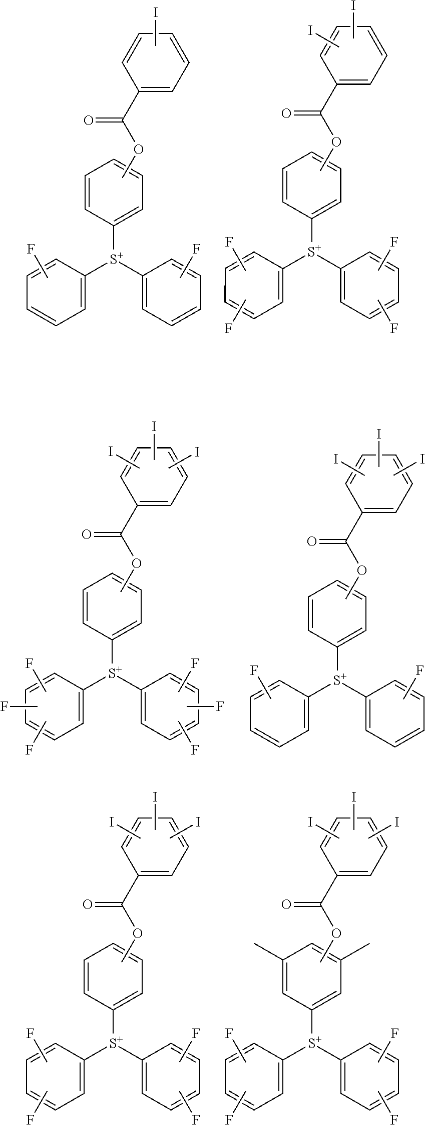

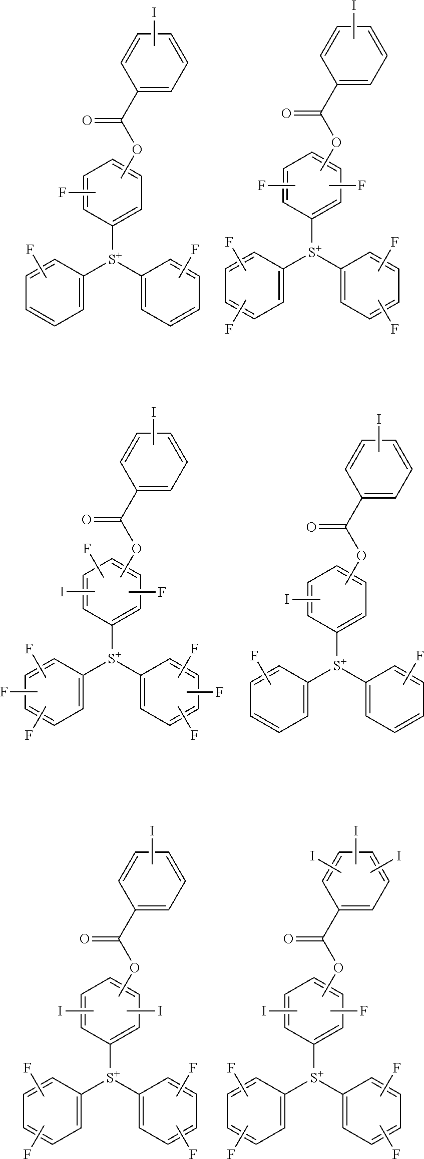

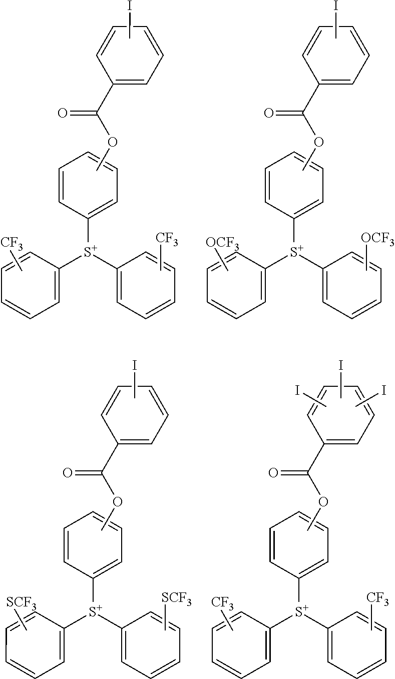

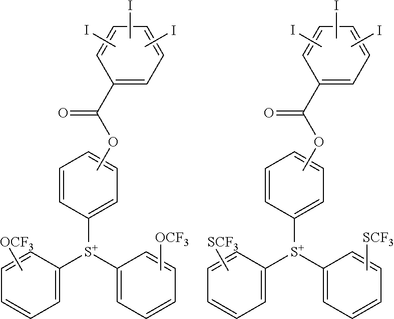

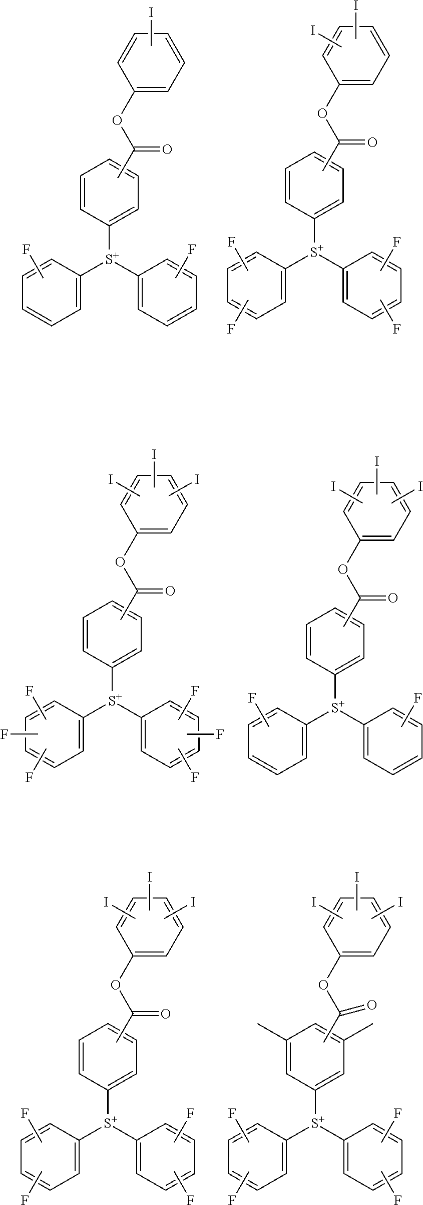

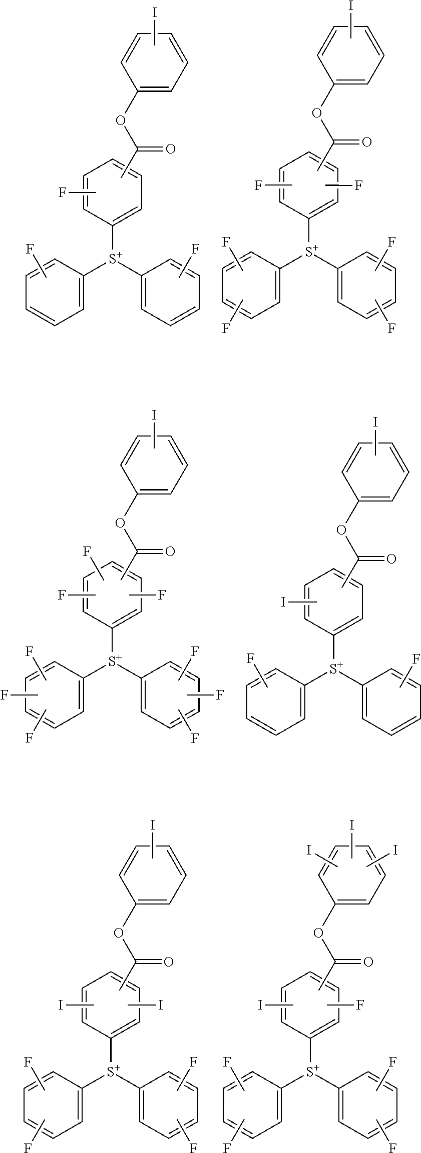

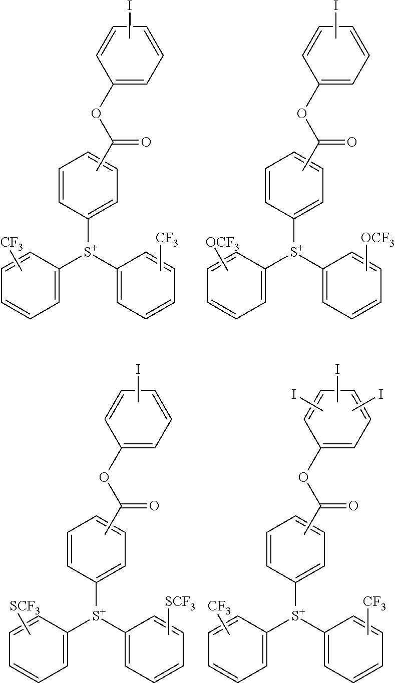

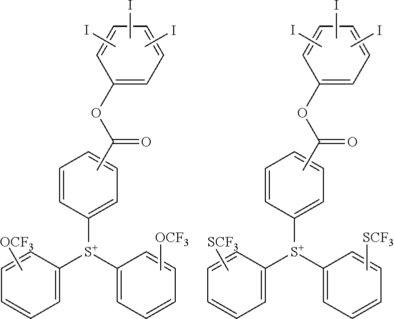

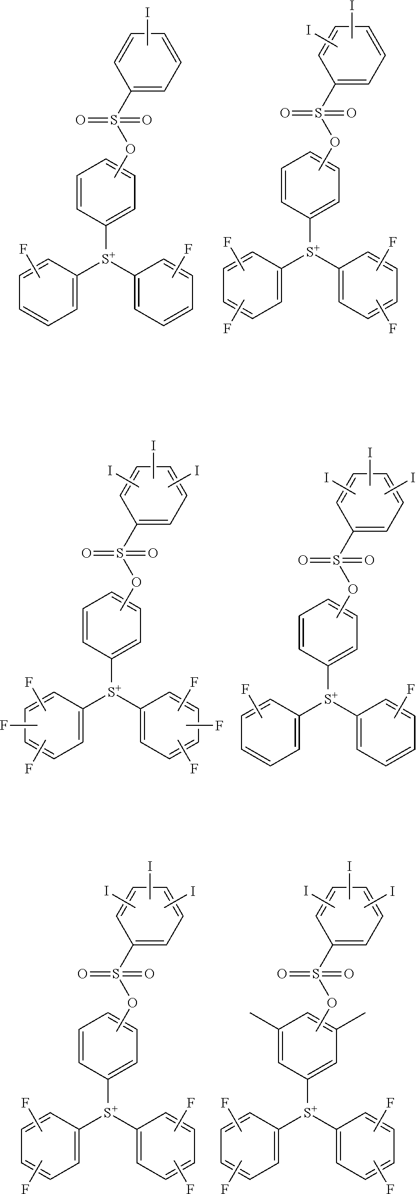

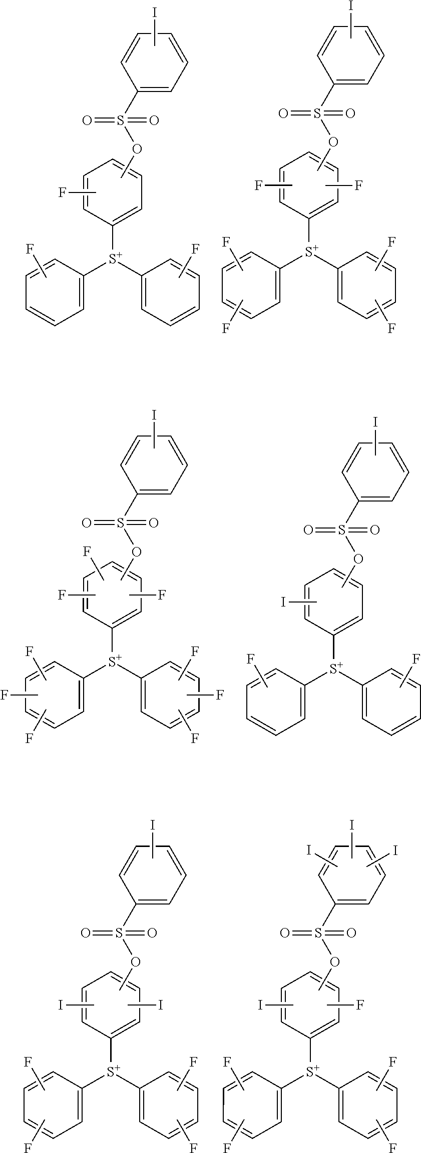

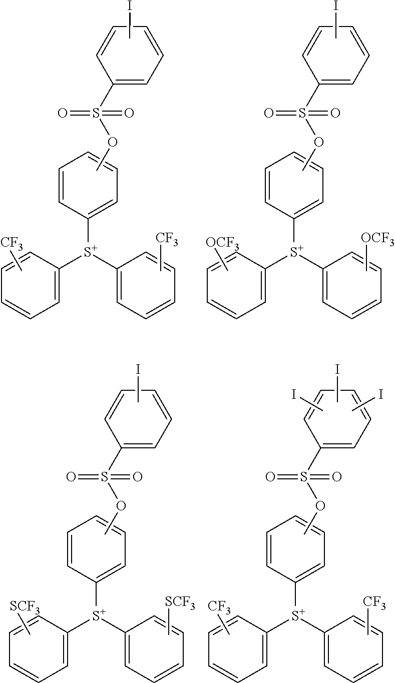

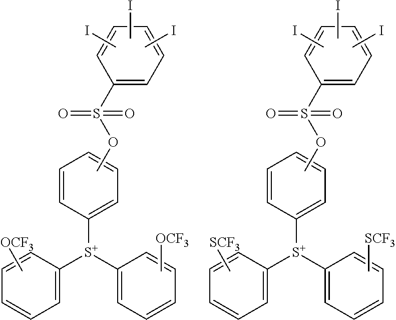

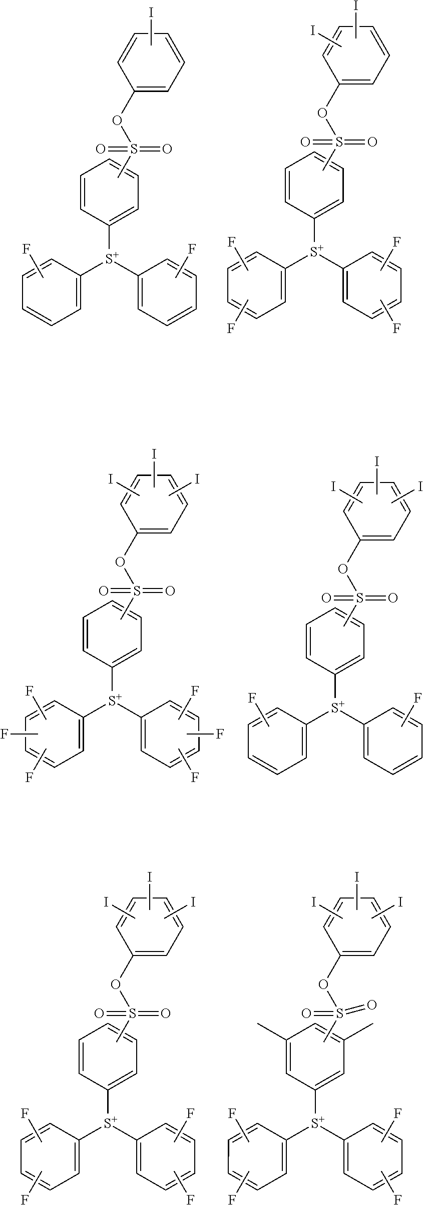

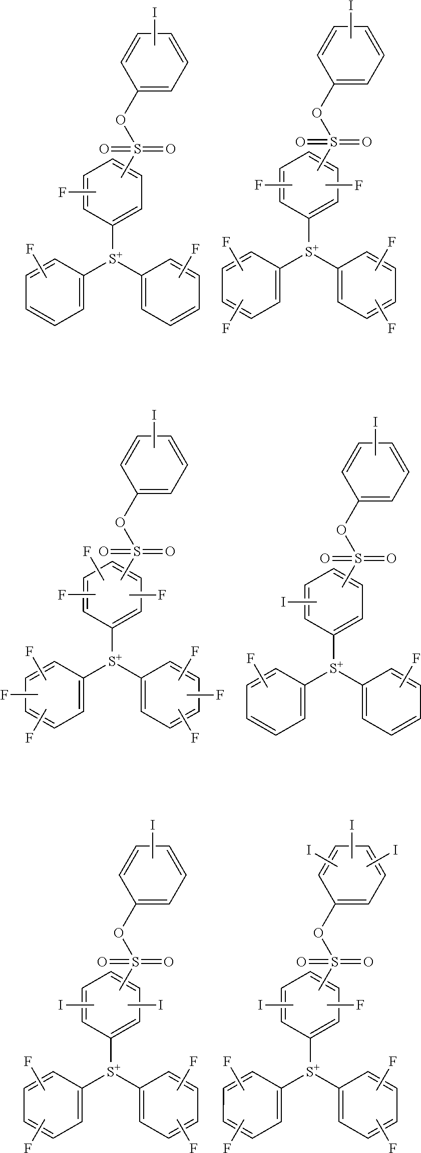

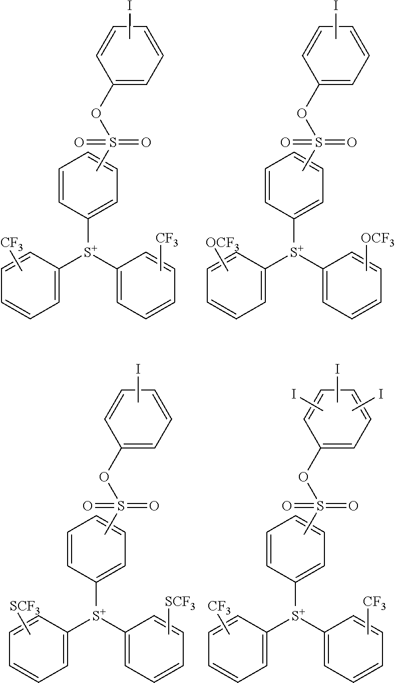

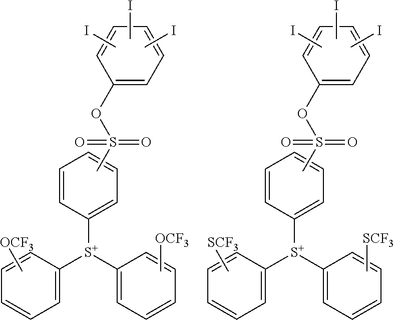

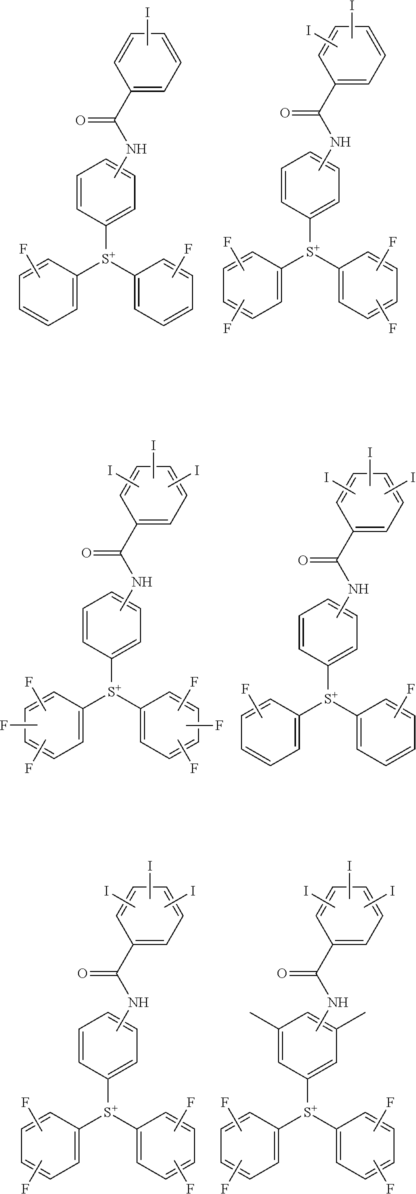

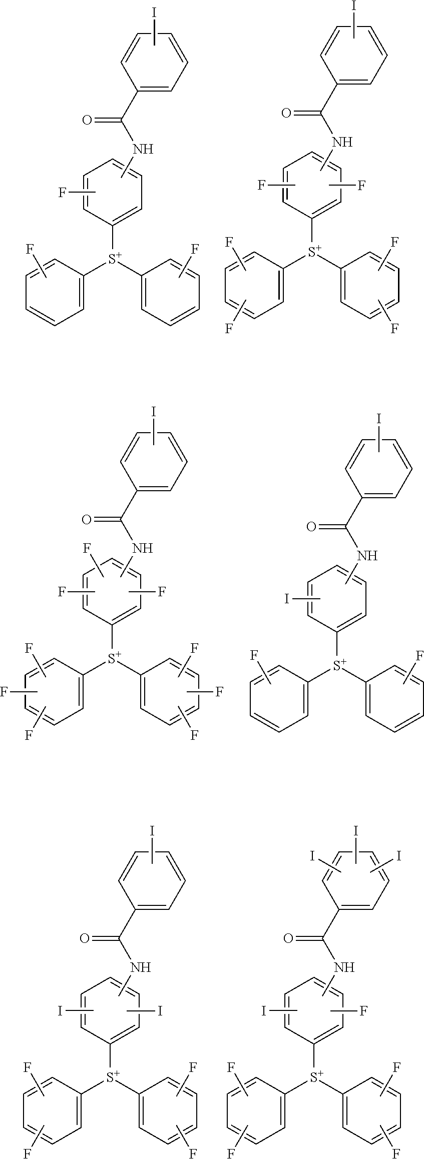

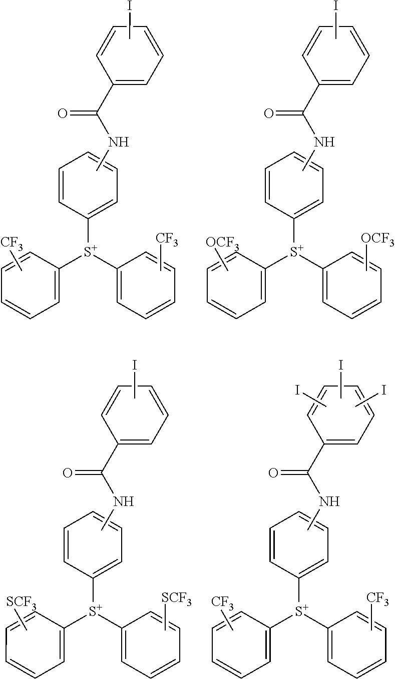

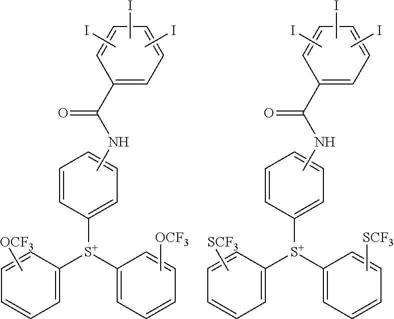

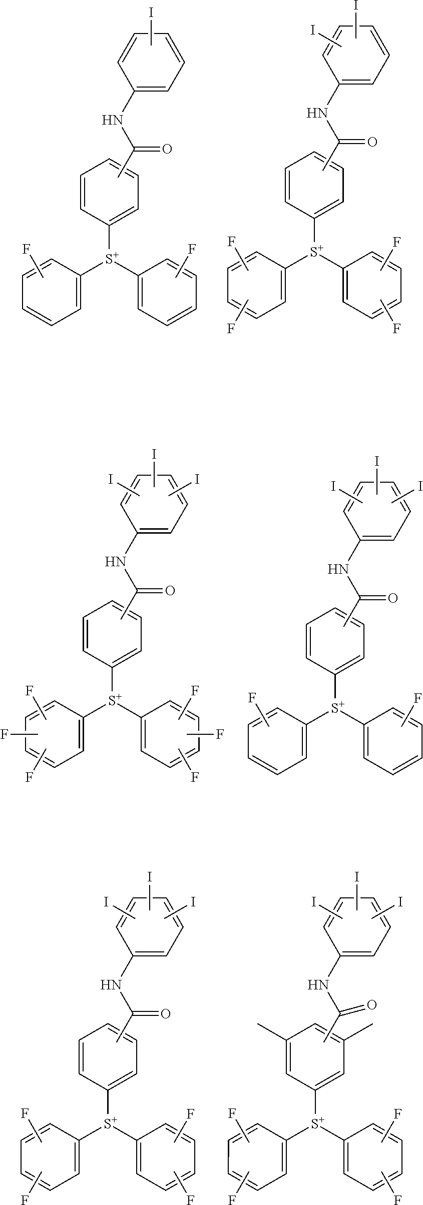

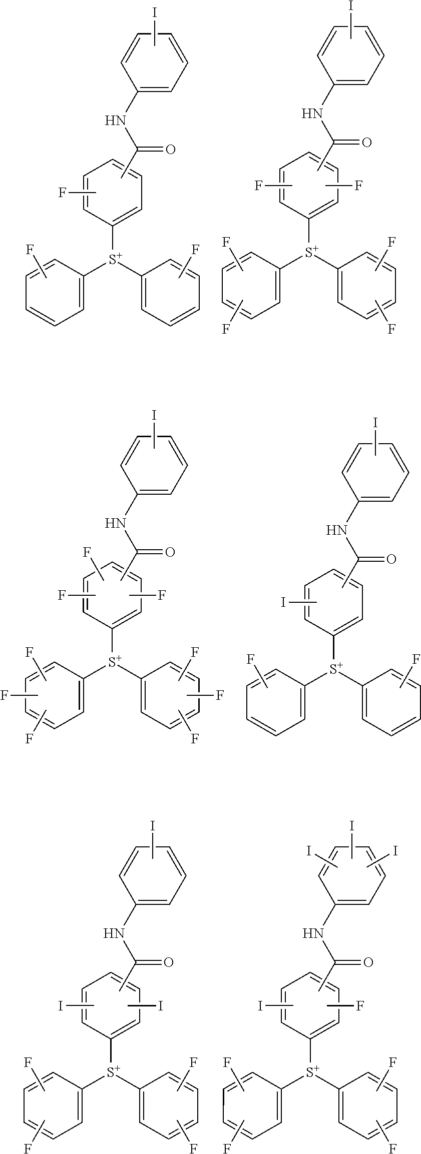

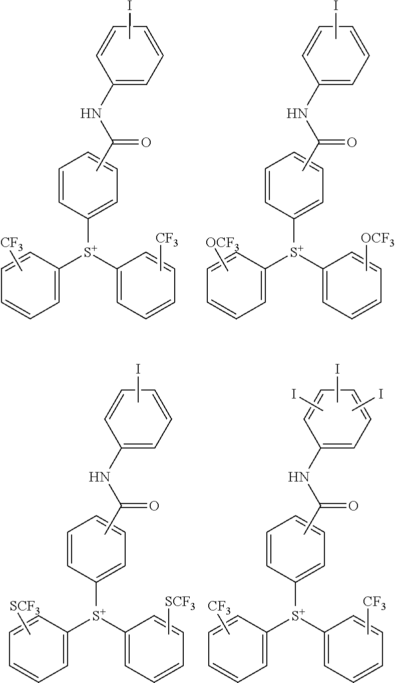

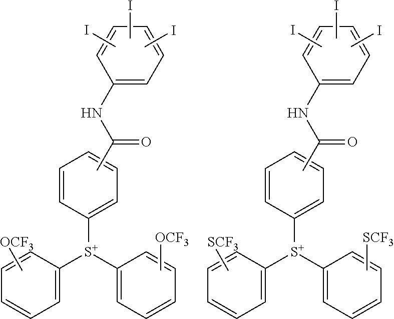

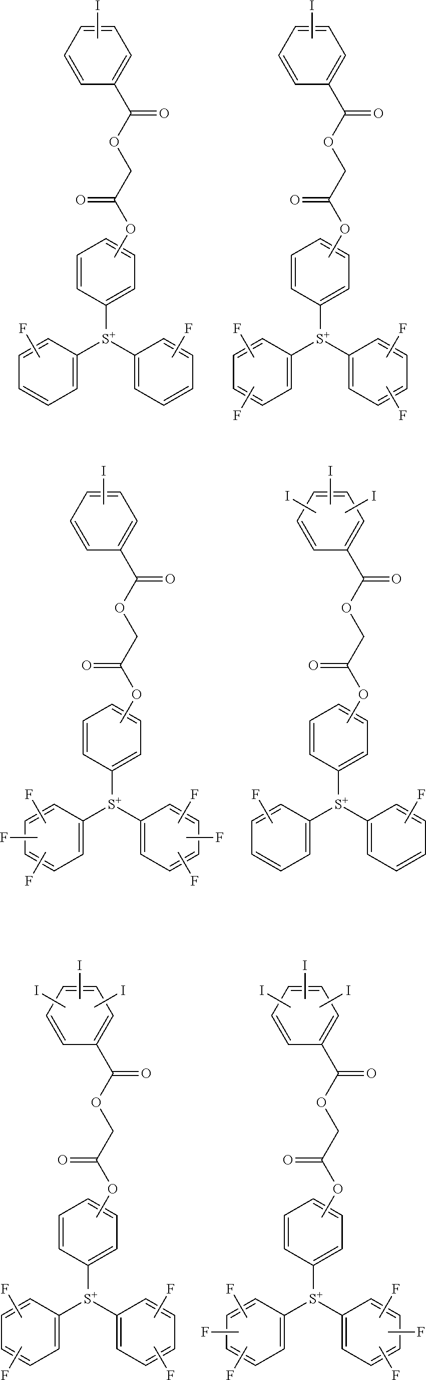

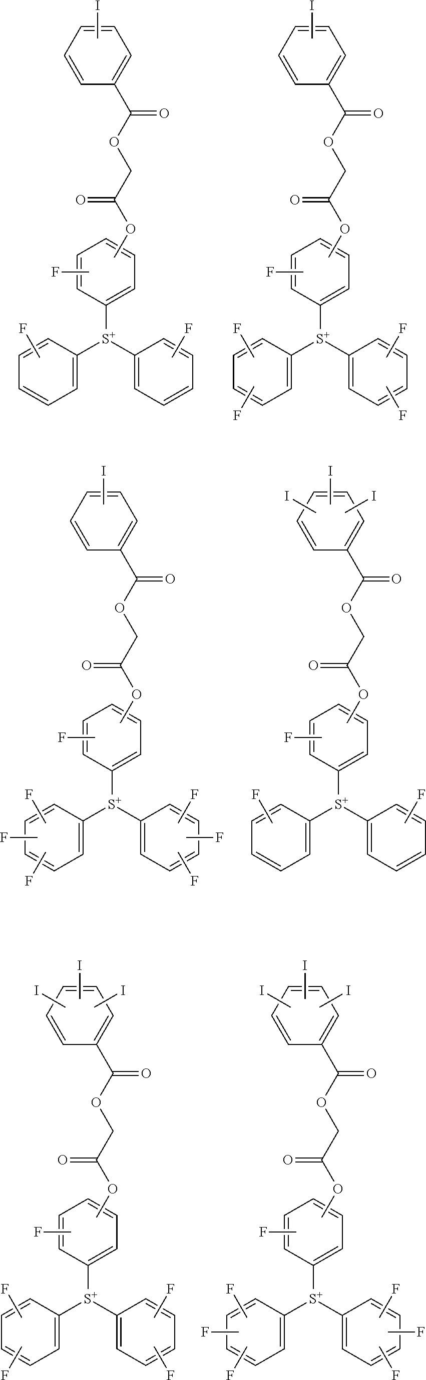

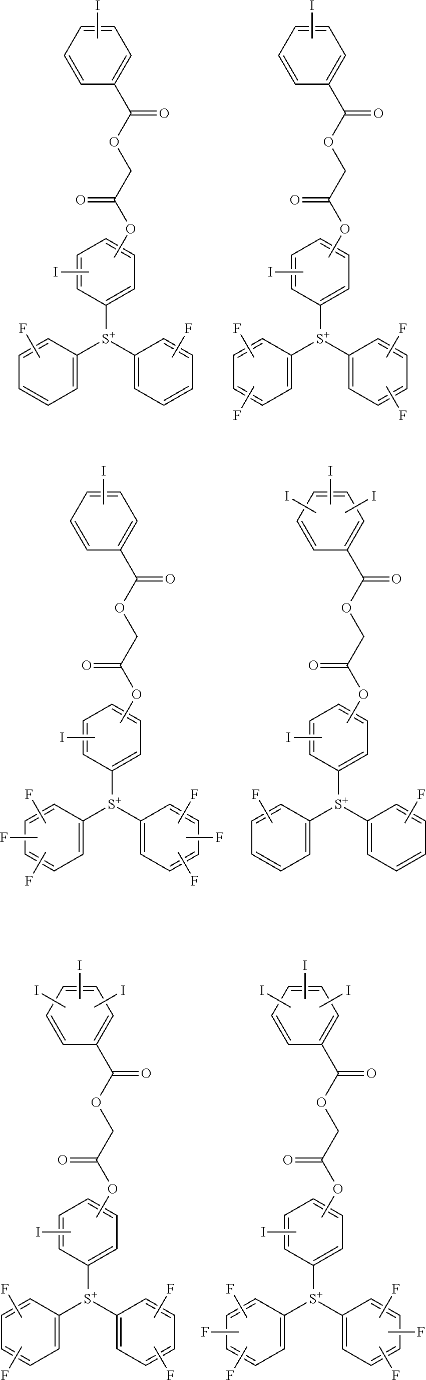

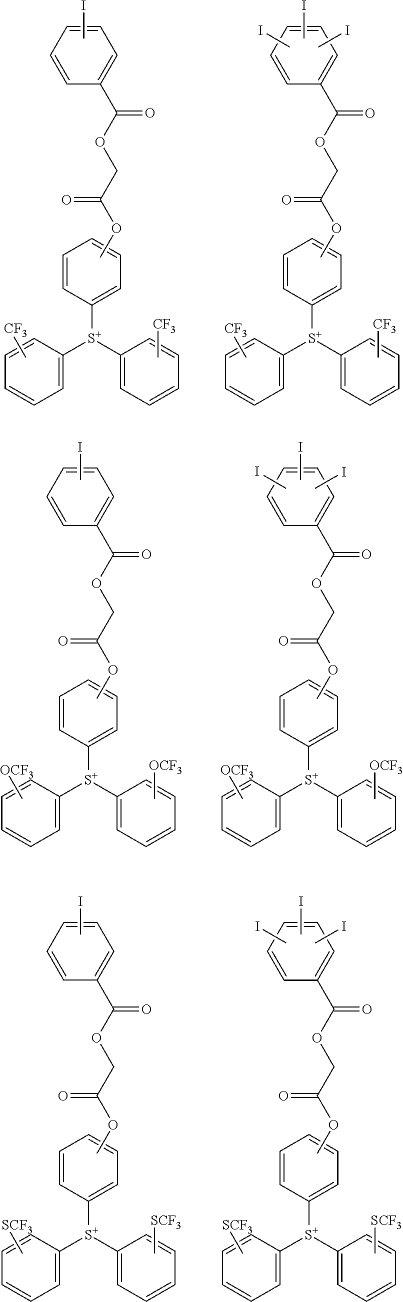

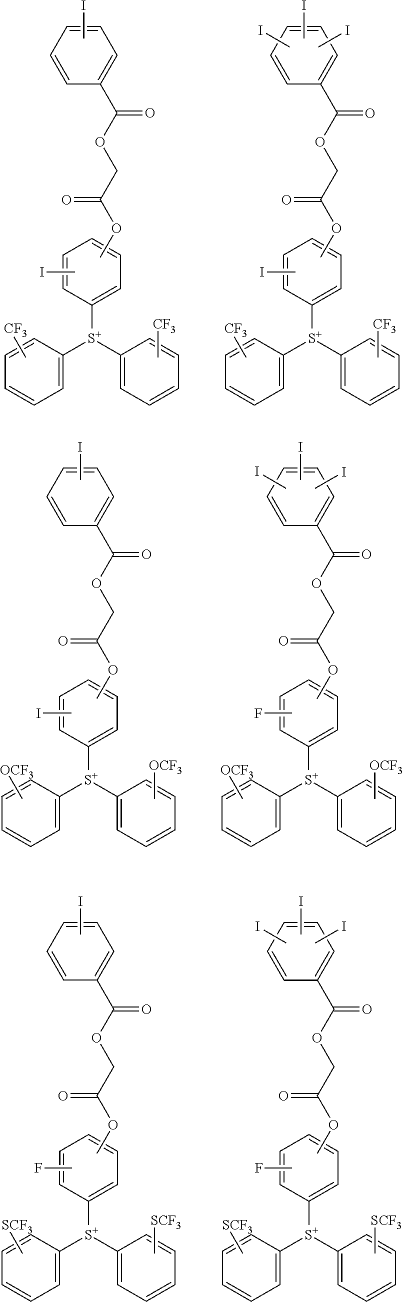

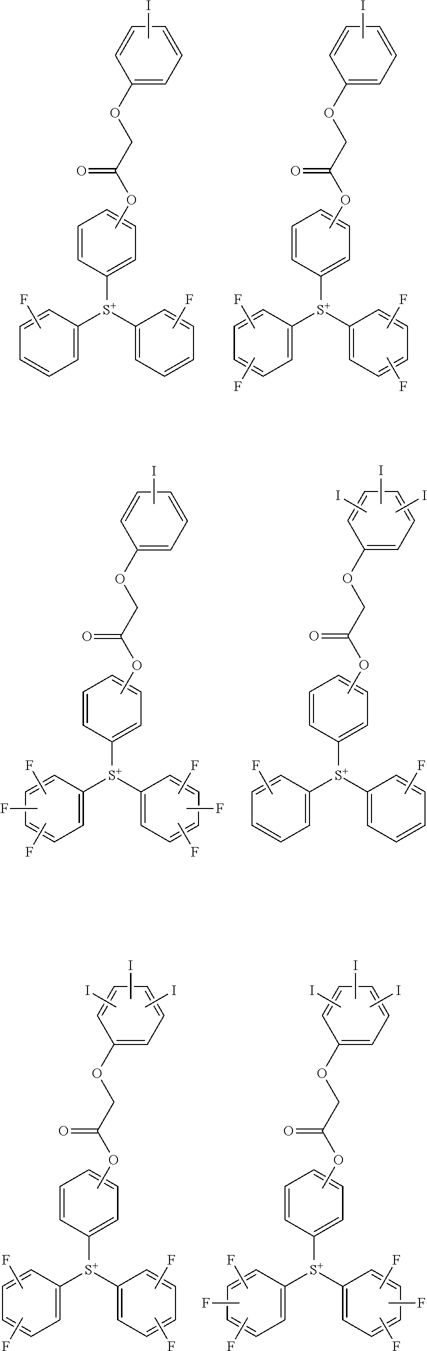

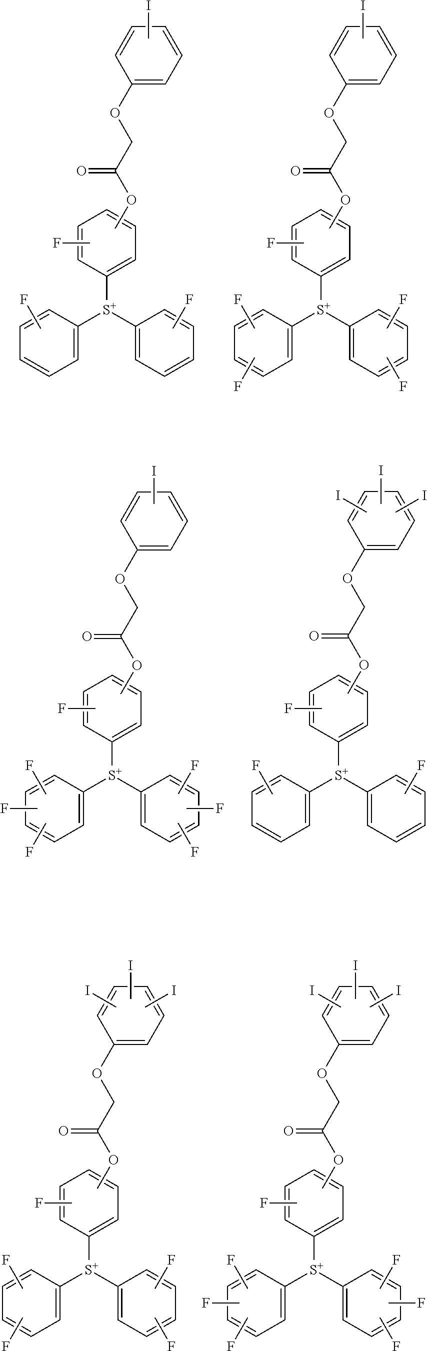

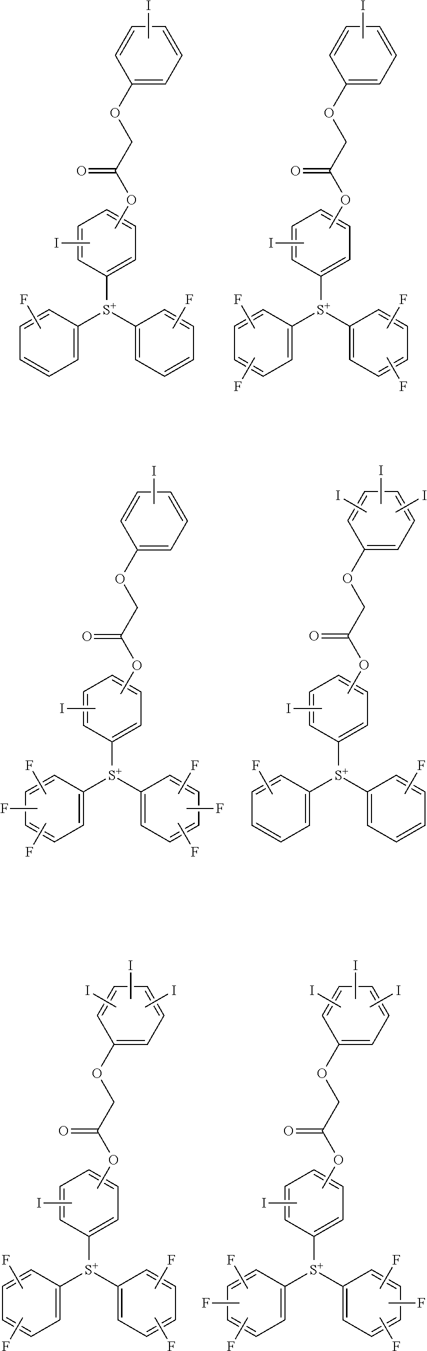

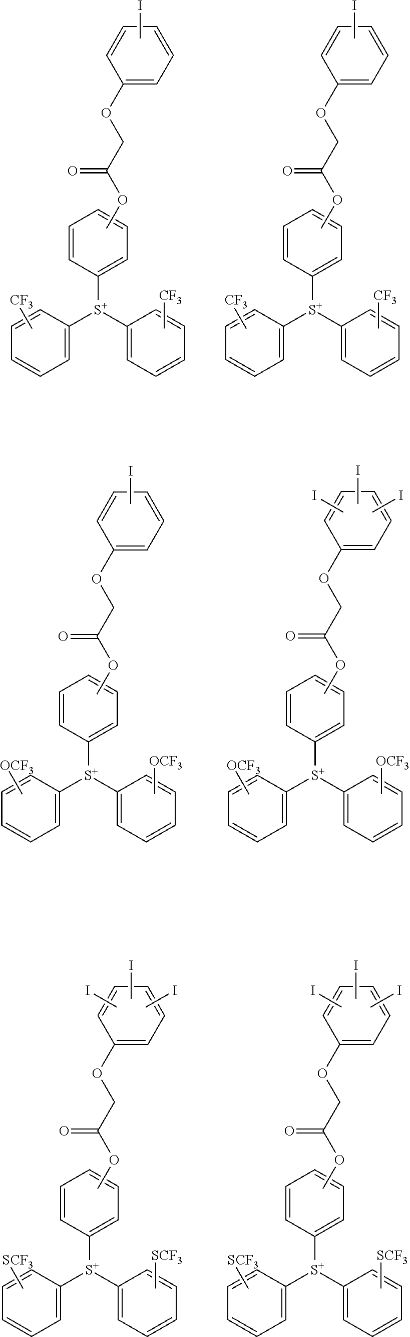

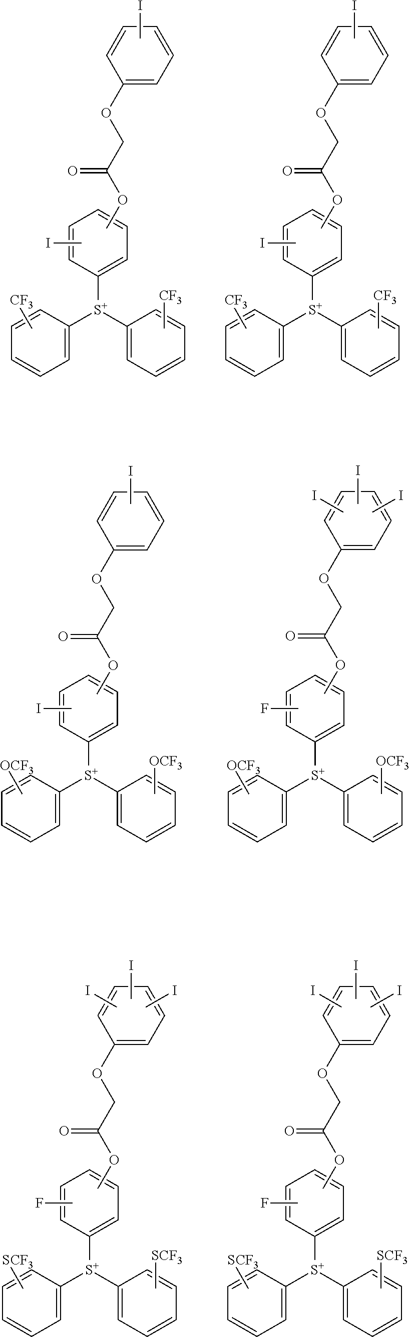

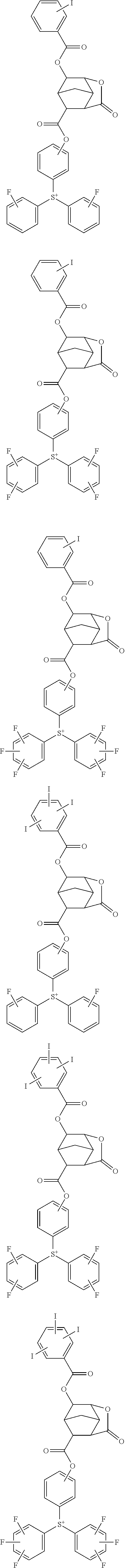

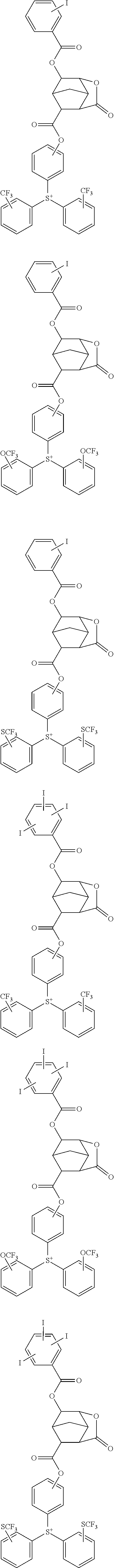

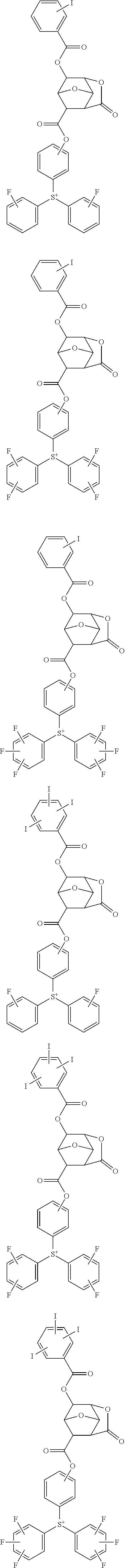

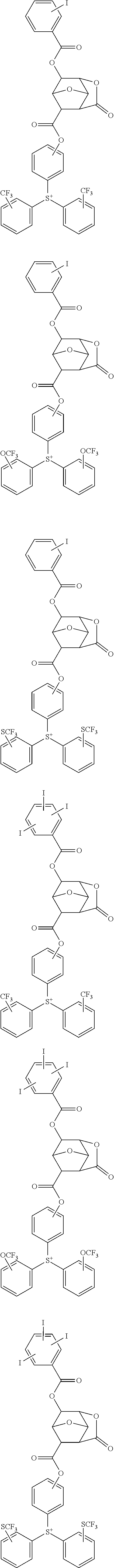

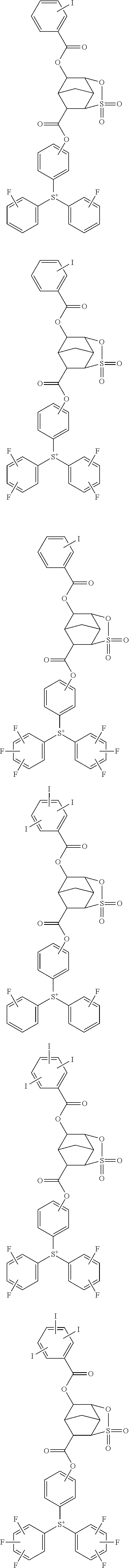

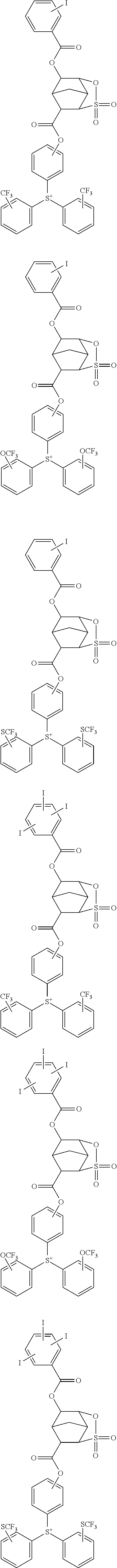

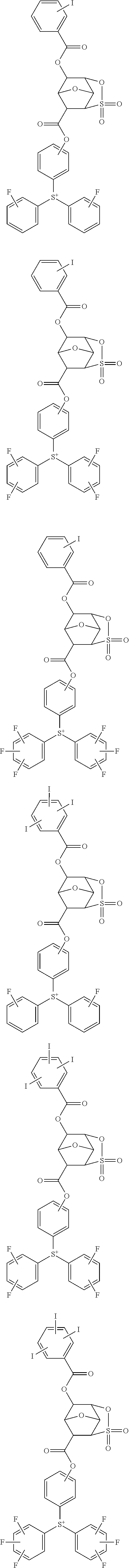

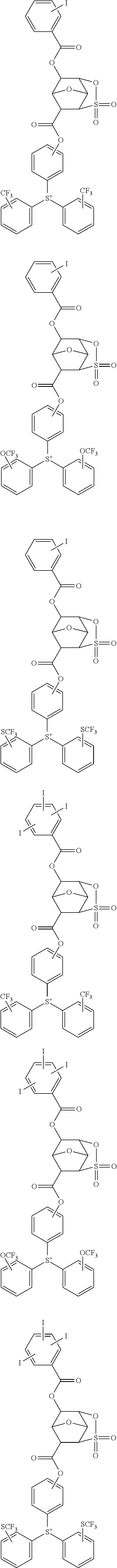

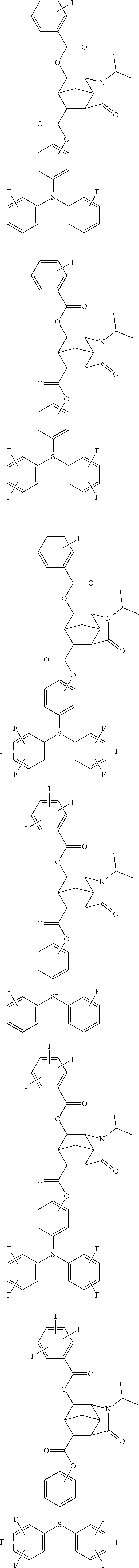

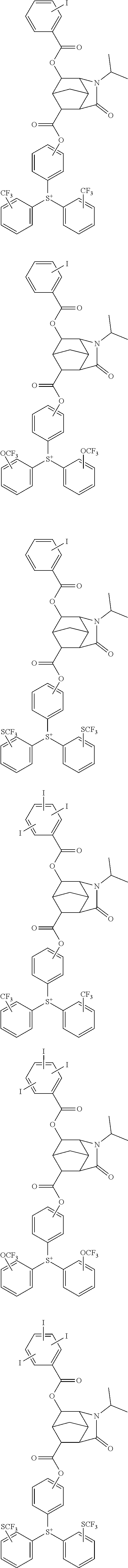

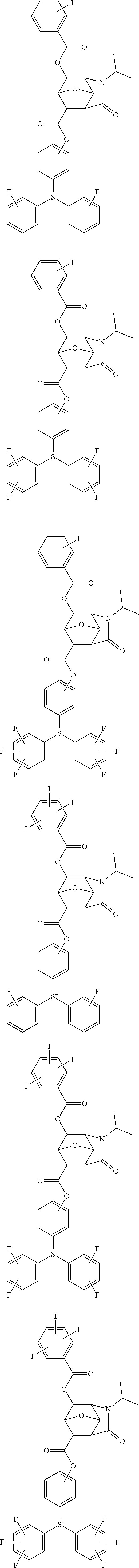

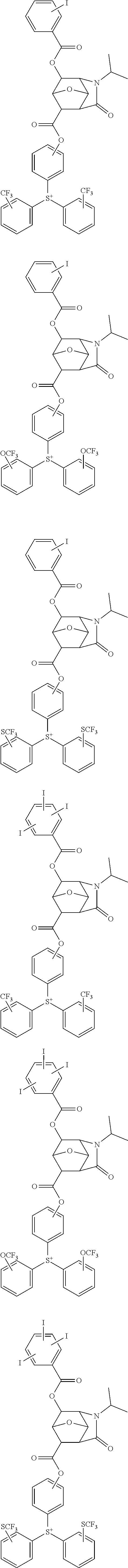

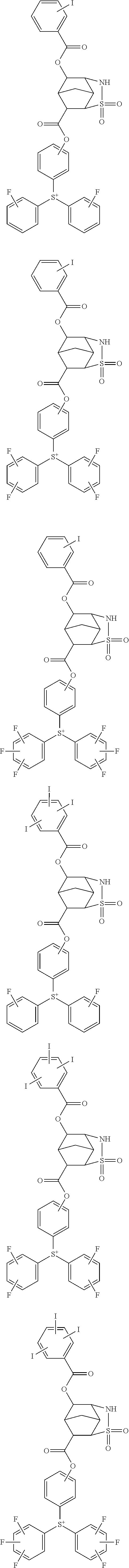

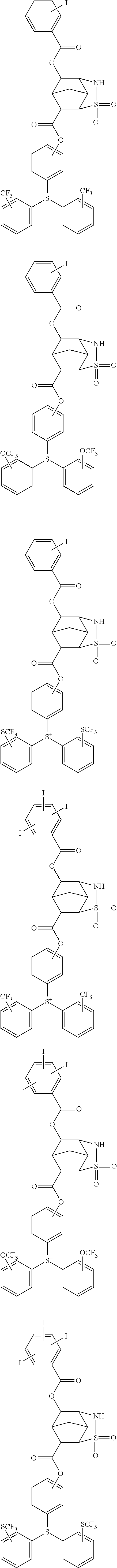

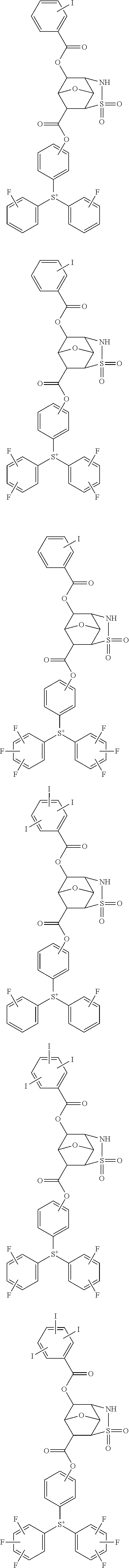

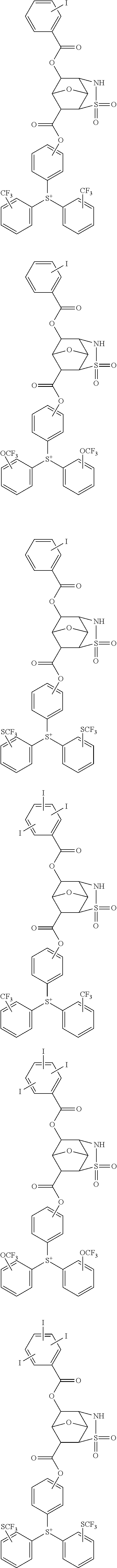

[chemical structure]

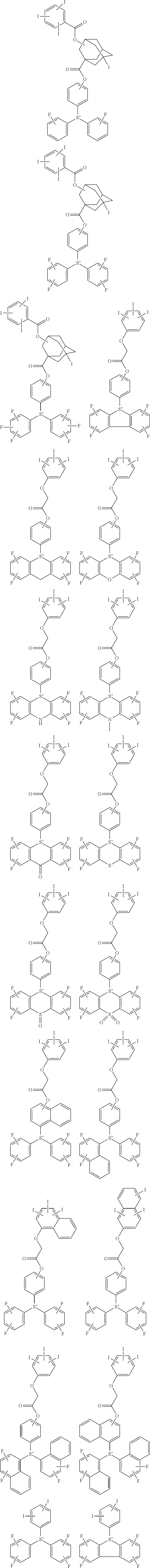

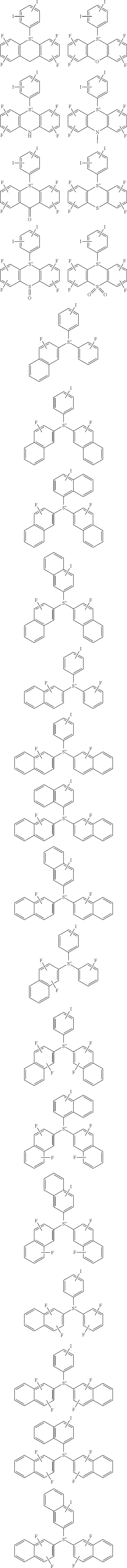

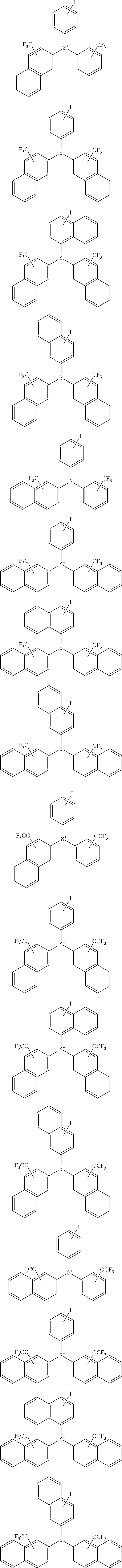

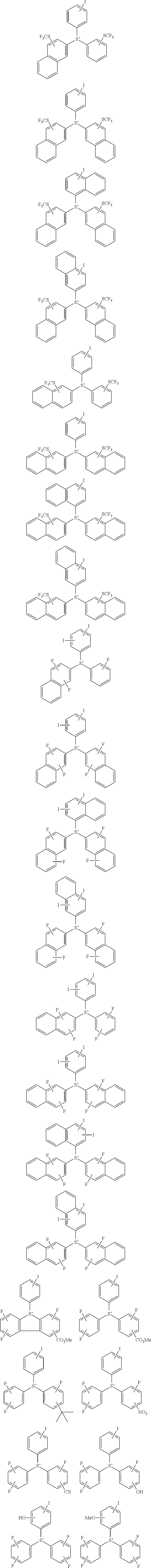

Specific examples of the iodonium cation include, but are not limited to, those described in paragraph [0181] of JP-A 2024-259.

Specific examples of the ammonium cation include those having the following formula (am-1).

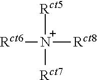

In the formula (am-1), R^{ct5} to R^{ct8} are each independently a C₁-C₄₀ hydrocarbyl group which may contain a heteroatom. R^{ct5} and R^{ct6} may be bonded to each other to form a ring together with a nitrogen atom to which they are bonded. Specific examples of the hydrocarbyl group include those similar to those exemplified as the hydrocarbyl group of R¹ and R² in the description of the formula (1).

Specific examples of the ammonium cation having the formula (am-1) include, but are not limited to, those listed below.

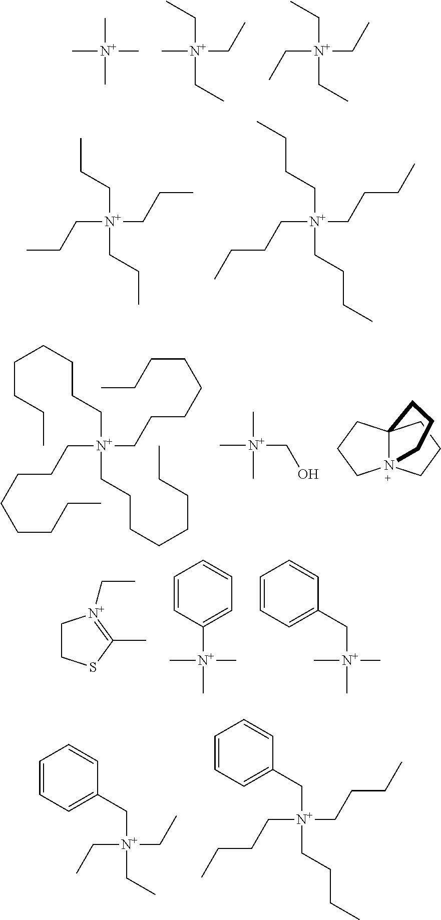

Examples of specific structures of the repeat units c1 to c5 include any combination of the anion and the cation described above.

Among the repeat units c1 to c5, the repeat units c2 to c5 are preferable from a viewpoint of controlling acid diffusion, the repeat units c2, c4, and c5 are more preferable from a viewpoint of acid strength of a generated acid, and the repeat unit c2 is still more preferable from a viewpoint of solvent solubility.

The polymer may contain a repeat unit having a structure in which a hydroxy group is protected by an acid-unstable group (hereinafter, also referred to as a repeat unit d). The repeat unit d is not particularly limited as long as it has one or more structures in which a hydroxy group is protected, and a protecting group is decomposed by an action of an acid to generate a hydroxy group, but preferably has the following formula (d1):

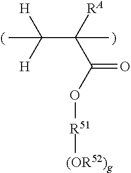

In the formula (d1), R^{A} is a hydrogen atom, a fluorine atom, a methyl group, or a trifluoromethyl group. R⁵¹ is a (g+1)-valent C₁-C₃₀ hydrocarbon group which may contain a heteroatom. R³² is an acid-unstable group. g is 1, 2, 3, or 4.

In the formula (d1), the acid-unstable group of R² only needs to be a group that performs deprotection by an action of an acid to generate a hydroxy group. The structure of R⁵² is not particularly limited, but is preferably an acetal structure, a ketal structure, a hydrocarbyloxycarbonyl group, a hydrocarbyloxymethyl group having the following formula (d2), or the like, and particularly preferably a hydrocarbyloxymethyl group having the following formula (d2).

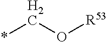

wherein * represents a bond, and R⁵³ is a C₁-C₁₅ hydrocarbyl group.

Specific examples of the acid-unstable group of R⁵², the hydrocarbyloxymethyl group of the formula (d2), and the repeat unit d include those similar to those exemplified in description of a repeat unit d described in JP-A 2020-111564.

The polymer may contain a repeat unit e derived from indene, benzofuran, benzothiophene, acenaphthylene, chromone, coumarin, norbornadiene, or a derivative thereof. Specific examples of a monomer that provides the repeat unit e include, but are not limited to, those listed below.

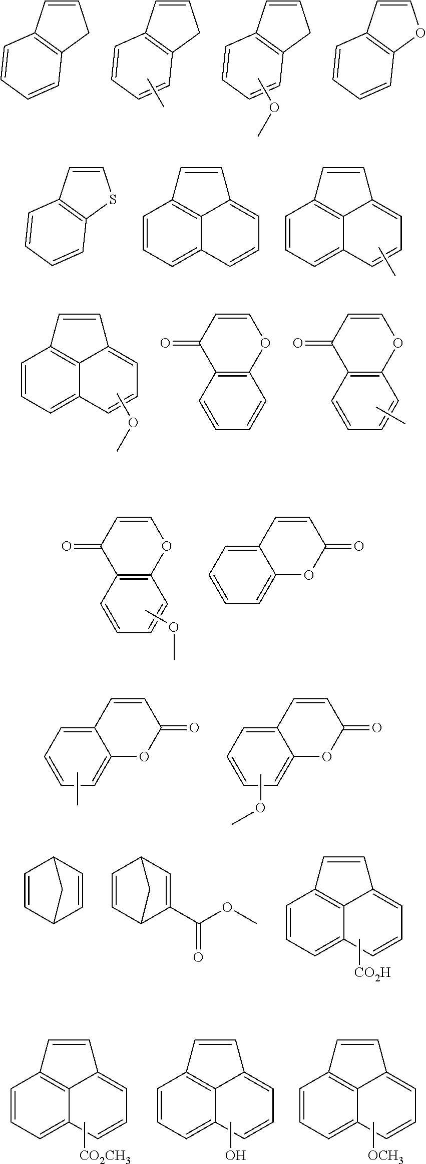

The polymer may contain a repeat unit f derived from indane, vinylpyridine, or vinylcarbazole.

In the polymer, content ratios of the repeat units a1, a2, a3, b1, b2, c1, c2, c3, c4, c5, d, e, and f are preferably 0≤a1≤0.8, 0≤a2≤0.8, 0≤a3≤0.6, 0≤b1≤0.8, 0≤b2≤0.5, 0≤c1≤0.4, 0≤c2≤0.4, 0≤c3≤0.4, 0≤c4≤0.4, 0≤c5≤0.4, 0≤d≤0.3, 0≤e≤0.3, and 0≤f≤0.3, and more preferably 0≤a1≤0.7, 0≤a2≤0.7, 0≤a3≤0.5, 0≤b1≤0.7, 0≤b2≤0.4, 0≤c1≤0.3, 0≤c2≤0.3, 0≤c3≤0.3, 0≤c4≤0.3, 0≤c5≤0.3, 0≤d≤s 0.2, 0≤e≤0.2, and 0≤f≤0.2. Provided that a1+a2+a3+b1+b2+c1+c2+c3+c4+d+e+f≤1.0.

A weight average molecular weight (Mw) of the polymer is preferably 1000 to 500,000, and more preferably 3000 to 100,000. When Mw is in this range, sufficient etching resistance is obtained, and there is no possibility of a decrease in resolution due to inability to ensure a difference in dissolution rate between before and after exposure. Note that, in the present invention, Mw is a value measured in terms of polystyrene by gel permeation chromatography (GPC) using tetrahydrofuran (THF) or N,N-dimethylformamide (DMF) as a solvent.

In a molecular weight distribution (Mw/Mn) of the polymer, an influence of Mw/Mn tends to increase as a pattern rule is finer. Therefore, in order to obtain a resist composition suitably used for fine pattern dimensions, Mw/Mn preferably has a narrow dispersion value of 1.0 to 2.0. Within the above range, the amount of a polymer having a low molecular weight or a high molecular weight is small, and there is no possibility that a foreign matter is observed on a pattern or the shape of the pattern is deteriorated after exposure.

Examples of a method for synthesizing the polymer include a method in which a monomer that provides the above-described repeat unit is added to an organic solvent with a radical polymerization initiator and heated to be polymerized.

Specific examples of the organic solvent used during polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The addition amount of these initiators is preferably 0.01 to 25 mol % based on the total moles of monomers to be polymerized. A reaction temperature is preferably 50 to 150° C., and more preferably 60 to 100° C. A reaction time is preferably 2 to 24 hours, and more preferably 2 to 12 hours from a viewpoint of production efficiency.

The polymerization initiator may be added to the monomer solution, and the mixture may be supplied to a reaction vessel, or an initiator solution may be prepared separately from the monomer solution, and the initiator solution and the monomer solution may be each independently supplied to the reaction vessel. The monomer solution and the initiator solution are preferably each independently prepared and dropwise added from a viewpoint of quality control because there is a possibility that a polymerization reaction proceeds by radicals generated from an initiator during a standby time to generate an ultrahigh polymer. An acid-unstable group introduced into the monomer may be used as it is, or protection or partial protection may be performed after polymerization. In order to adjust a molecular weight, a known chain transfer agent such as dodecyl mercaptan or 2-mercaptoethanol may be used in combination. In this case, the addition amount of these chain transfer agents is preferably 0.01 to 20 mol % based on the total moles of monomers to be polymerized.

In a case of a monomer containing a hydroxy group, the hydroxy group may be replaced with an acetal group such as ethoxyethoxy, which can be easily deprotected by an acid during polymerization, and then deprotected by a weak acid and water after polymerization, or may be replaced with an acetyl group, a formyl group, a pivaloyl group, or the like, and then subjected to alkali hydrolysis after polymerization.

When hydroxystyrene or hydroxyvinylnaphthalene is copolymerized, hydroxystyrene or hydroxyvinylnaphthalene and another monomer may be heated to be polymerized by adding a radical polymerization initiator in an organic solvent, but acetoxystyrene or acetoxyvinylnaphthalene may be used, and an acetoxy group may be deprotected by alkali hydrolysis after polymerization to obtain polyhydroxystyrene or hydroxypolyvinylnaphthalene.

Specific examples of a base during alkali hydrolysis include ammonia water and triethylamine. A reaction temperature is preferably −20 to 100° C., and more preferably 0 to 60° C. A reaction time is preferably 0.2 to 100 hours, and more preferably 0.5 to 20 hours.

Note that the amount of each monomer in the monomer solution only needs to be appropriately set, for example, so as to have the above-described preferable content ratios of the repeat units.

Regarding a polymer obtained by the manufacturing method, a reaction solution obtained by the polymerization reaction may be used as a final product, or a powder obtained through a purification step such as a reprecipitation method in which a polymerization liquid is added to a poor solvent to obtain a powder may be used as a final product. However, it is preferable to use a polymer solution obtained by dissolving a powder obtained by the purification step in a solvent as a final product from a viewpoint of operation efficiency and quality stabilization.

Specific examples of the solvent used at this time include: ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; alcohols such as diacetone alcohol (DAA); high boiling point alcohol solvents such as diethylene glycol, propylene glycol, glycerin, 1,4-butanediol, and 1,3-butanediol; and a mixed solvent thereof, described in paragraphs [0144] to [0145] of JP-A 2008-111103.

A concentration of the polymer in the polymer solution is preferably 0.01 to 30 wt %, and more preferably 0.1 to 20 wt %.

The reaction solution and the polymer solution are preferably subjected to filter filtration. By performing filter filtration, foreign matters and gels that may cause defects can be removed, and it is effective in terms of quality stabilization.

Examples of a material of the filter used for the filter filtration include a fluorocarbon-based material, a cellulose-based material, a nylon-based material, a polyester-based material, and a hydrocarbon-based material. In a step of filtering a resist composition, a filter formed of a fluorocarbon-based material called Teflon®, a hydrocarbon-based material such as polyethylene or polypropylene, or nylon is preferable. A pore size of the filter can be appropriately selected according to a target cleanliness, but is preferably 100 nm or less, and more preferably 20 nm or less. These filters may be used singly or in combination of two or more types thereof. In a filtration method, a solution may be caused to pass only once, but it is more preferable to perform filtration a plurality of times by circulating the solution. The filtration step can be performed in any order and any number of times in the polymer manufacturing process, but it is preferable to filter the reaction solution after a polymerization reaction, the polymer solution, or both.

The base polymer (B) may be used singly, or two or more polymers having different composition ratios, Mw, and/or Mw/Mn may be used in combination. In addition, the base polymer (B) may contain a hydrogenated ring-opened metathesis polymer in addition to the polymer, and as the hydrogenated ring-opened metathesis polymer, those described in JP-A 2003-66612 can be used.

### Organic Solvent (C)

The chemically amplified resist composition of the present invention may contain an organic solvent as a component (C). The organic solvent (C) is not particularly limited as long as it can dissolve the components described above and components described below. Specific examples of such an organic solvent include: ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; ketoalcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; and a mixed solvent thereof.

Among these organic solvents, 1-ethoxy-2-propanol, PGMEA, cyclohexanone, GBL, ethyl lactate, DAA, and a mixed solvent thereof, in which solubility of the base polymer as the component (B) is particularly excellent, are preferable.

The content of the organic solvent (C) in the chemically amplified resist composition of the present invention is preferably 200 to 5000 parts by weight, and more preferably 400 to 3500 parts by weight, per 80 parts by weight of the base polymer (B). The organic solvent (C) may be used singly or in combination of two or more types thereof.

### Photoacid Generator (D)

The chemically amplified resist composition of the present invention may contain a photoacid generator as a component (D). The photoacid generator is not particularly limited as long as it is a compound that generates a strong acid by irradiation with a high energy ray.

Examples of a suitable photoacid generator include those having the following formula (2) or (3).

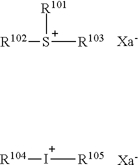

In the formula (2), R¹⁰¹ to R¹⁰⁵ are each independently a halogen atom or a C₁-C₂₀ hydrocarbyl group which may contain a heteroatom. Any two of R¹⁰¹, R¹⁰², and R¹⁰³ may be bonded to each other to form a ring together with a sulfur atom to which they are bonded. Specific examples of the hydrocarbyl group include those similar to those exemplified as the hydrocarbyl group of R¹ and R² in the description of the formula (1).

Specific examples of the cation of the sulfonium salt having the formula (2) include those exemplified as specific examples of the cation of the sulfonium salt having the formula (1), those described in paragraphs [0102] to [0125] of JP-A 2024-3744, those described in paragraphs [0044] to [0049] of WO 2024/128017, those described in paragraphs [0035] to [0046] of JP 7491173, and those exemplified as specific examples of the sulfonium cation having the formula (sulfo-1), but are not limited thereto. Specific examples of the cation of the iodonium salt having the formula (3) include, but are not limited to, those described in paragraph [0181] of JP-A 2024-259.

In the formulas (2) and (3), Xa⁻ is an anion of a strong acid. Specific examples of the anion of a strong acid include those having any of the following formulas (c1-1) to (c1-5). Fluorine-containing aromatic sulfonic acid anions described in paragraphs [0029] to [0034] of JP 6648726, paragraphs [0264] to [0267] of JP-A 2024 077330, and paragraphs [0040] to [0071] of JP-A 2025 000201 can also be used as the anion of a strong acid.

As the photoacid generator as the component (D), those having the following formula (4) are also preferable.

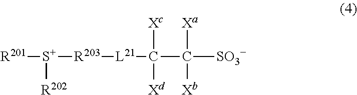

In the formula (4), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰², and R²⁰³ may be bonded to each other to form a ring together with a sulfur atom to which they are bonded.

The C₁-C₃₀ hydrocarbyl group of R²⁰¹ and R²⁰² may be saturated or unsaturated, and may be linear, branched, or cyclic. Specific examples thereof include: a C₁-C₃₀ alkyl group such as a methyl group, an ethyl group, a n-propyl group, an isopropyl group, a n-butyl group, an isobutyl group, a sec-butyl group, a tert-butyl group, a tert-pentyl group, a n-pentyl group, a n-hexyl group, a n-octyl group, a 2-ethylhexyl group, a n-nonyl group, or a n-decyl group; a C₃-C₃₀ cyclic saturated hydrocarbyl group such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo [5.2.1.0^{2,6}] decyl group, or an adamantyl group; a C₆-C₃₀ aryl group such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, a tert-butylnaphthyl group, or an anthracenyl group; and a group obtained by combining these groups. Some or all of the hydrogen atoms of the hydrocarbyl group may be replaced with a group containing a heteroatom such as an oxygen atom, a sulfur atom, a nitrogen atom, or a halogen atom, some of the (—CH₂-)s in the hydrocarbyl group may be replaced with a group containing a heteroatom such as an oxygen atom, a sulfur atom, or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

The C₁-C₃₀ hydrocarbylene group of R²⁰³ may be saturated or unsaturated, and may be linear, branched, or cyclic. Specific examples thereof include: a C₁-C₃₀ alkanediyl group such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, or a heptadecane-1,17-diyl group; a C₃-C₃₀ cyclic saturated hydrocarbylene group such as a cyclopentanediyl group, a cyclohexanediyl group, a norbornanediyl group, or an adamantanediyl group; and an arylene group such as a phenylene group, a methylphenylene group, an ethylphenylene group, a n-propylphenylene group, an isopropylphenylene group, a n-butylphenylene group, an isobutylphenylene group, a sec-butylphenylene group, a tert-butylphenylene group, a naphthylene group, a methylnaphthylene group, an ethylnaphthylene group, a n-propylnaphthylene group, an isopropylnaphthylene group, a n-butylnaphthylene group, an isobutylnaphthylene group, a sec-butylnaphthylene group, or a tert-butylnaphthylene group. Some or all of the hydrogen atoms of the hydrocarbylene group may be replaced with a group containing a heteroatom such as an oxygen atom, a sulfur atom, a nitrogen atom, or a halogen atom, some of the (—CH₂-)s in the hydrocarbylene group may be replaced with a group containing a heteroatom such as an oxygen atom, a sulfur atom, or a nitrogen atom, and as a result, the hydrocarbylene group may contain a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like. The heteroatom is preferably an oxygen atom.

In the formula (4), L²¹ is a single bond, an ether bond, or a C₁-C₂₀ hydrocarbylene group which may contain a heteroatom. The hydrocarbylene group may be saturated or unsaturated, and may be linear, branched, or cyclic. Specific examples thereof include those similar to those exemplified as the hydrocarbylene group of R²⁰³

In the formula (4), X^{a}, X^{b}, X^{c}, and X^{d} are each independently a hydrogen atom, a fluorine atom, or a trifluoromethyl group. Note that at least one of X^{a}, X^{b}, X^{c}, and X^{d} is a fluorine atom or a trifluoromethyl group.

The photoacid generator having formula (4) preferably has the following formula (4′):

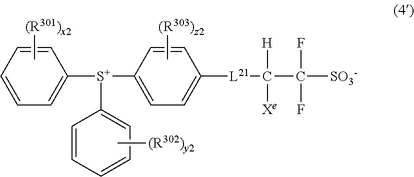

In the formula (4′), L²¹ is as defined above. X^{e} is a hydrogen atom or a trifluoromethyl group, and preferably a trifluoromethyl group. R³⁰¹, R³⁰², and R³⁰³ are each independently a hydrogen atom or a C₁-C₂₀ hydrocarbyl group which may contain a heteroatom. The hydrocarbyl group may be saturated or unsaturated, and may be linear, branched, or cyclic. Specific examples thereof include those similar to those exemplified as the hydrocarbyl group of R^{fa1} in the formula (c1-1-1). x2 and y2 are each independently 0, 1, 2, 3, 4, or 5, and z2 is 0, 1, 2, 3, or 4.

Specific examples of the photoacid generator having formula (4) include those similar to those exemplified as the photoacid generator having formula (2) in JP-A 2017-26980.

Among the photoacid generators, those containing an anion having the formula (c1-1-1) or (c1-4) are particularly preferable because they have small acid diffusion and excellent solubility in a solvent. In addition, those having the formula (4′) have extremely small acid diffusion, and are particularly preferable.

When the chemically amplified resist composition of the present invention contains the photoacid generator (D), the content thereof is preferably 0.1 to 40 parts by weight, and more preferably 0.5 to 20 parts by weight per 80 parts by weight of the base polymer (B). When the addition amount of the photoacid generator (D) is in the above range, resolution is favorable, and there is no possibility that a problem of foreign matters occurs after development of a resist film or during peeling of the resist film, which is preferable. The photoacid generator (D) may be used singly or in combination of two or more types thereof.

### Other Quencher (E)

The chemically amplified resist composition of the present invention may contain a quencher other than the component (A) (hereinafter, also referred to as another quencher) as a component (E).

Examples of the other quencher (E) include an onium salt having the following formula (5) or (6):

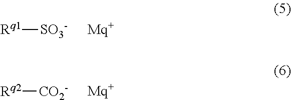

In the formula (5), R^{q1} is a hydrogen atom or a C₁-C₄₀ hydrocarbyl group which may contain a heteroatom, except a hydrocarbyl group in which a hydrogen atom bonded to a carbon atom at an α-position of a sulfo group is replaced with a fluorine atom or a fluoroalkyl group. In the formula (6), R^{q2} is a hydrogen atom or a C₁-C₄₀ hydrocarbyl group which may contain a heteroatom.

Specific examples of the C₁-C₄₀ hydrocarbyl group of R^{q1} include: a C₁-C₄₀ alkyl group such as a methyl group, an ethyl group, a n-propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group, or a n-decyl group; a C₃-C₄₀ cyclic saturated hydrocarbyl group such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo [5.2.1.0^{2,6}] decyl group, or an adamantyl group; and a C₆-C₄₀ aryl group such as a phenyl group, a naphthyl group, or an anthracenyl group. Some or all of the hydrogen atoms of the hydrocarbyl group may be replaced with a group containing a heteroatom such as an oxygen atom, a sulfur atom, a nitrogen atom, or a halogen atom, some of the (—CH₂-)s in the hydrocarbyl group may be replaced with a group containing a heteroatom such as an oxygen atom, a sulfur atom, or a nitrogen atom, and as a result, the hydrocarbyl group may contain a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic acid anhydride (—C(═O)—O—C(═O)—), a haloalkyl group, or the like.

Specific examples of the hydrocarbyl group of R^{q2} include, in addition to the substituents exemplified as specific examples of R^{q1}, a fluorinated saturated hydrocarbyl group such as a trifluoromethyl group or a trifluoroethyl group, and a fluorinated aryl group such as a pentafluorophenyl group or a 4-trifluoromethylphenyl group.

Specific examples of an anion of the onium salt having the formula (5) include, but are not limited to, those listed below.

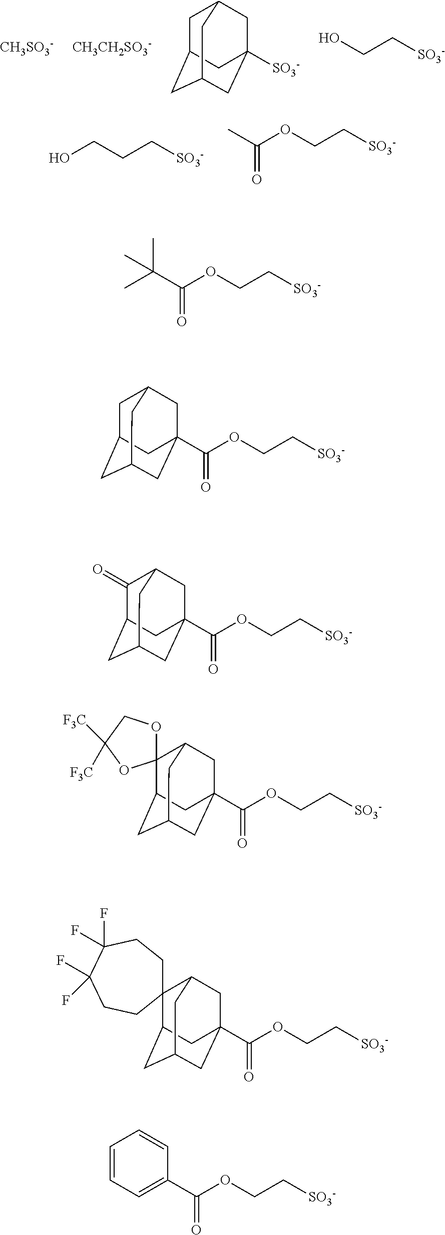

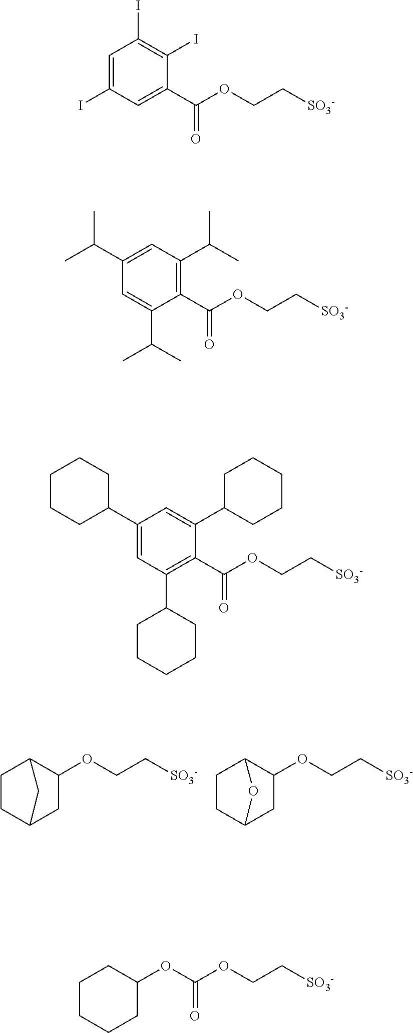

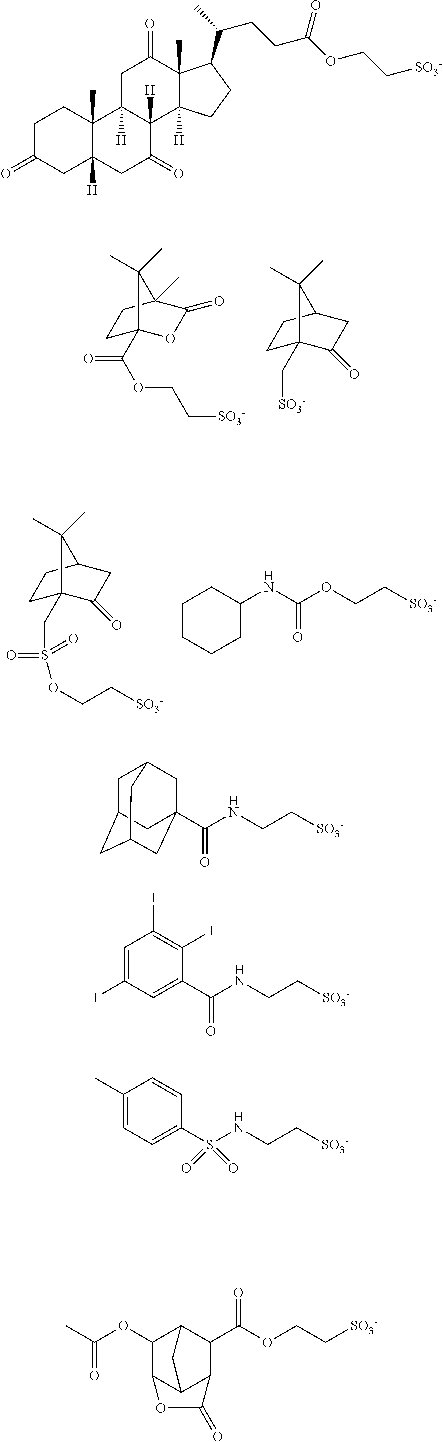

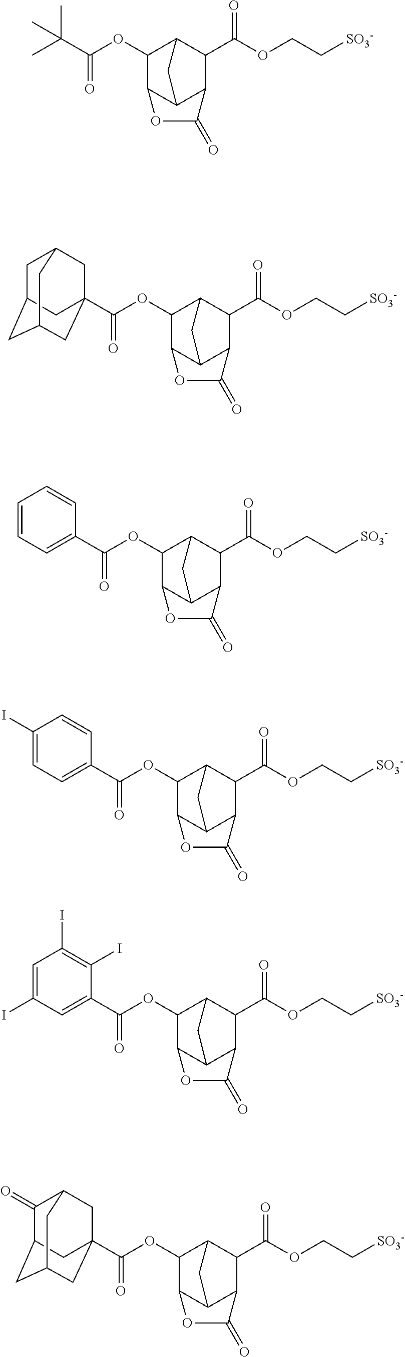

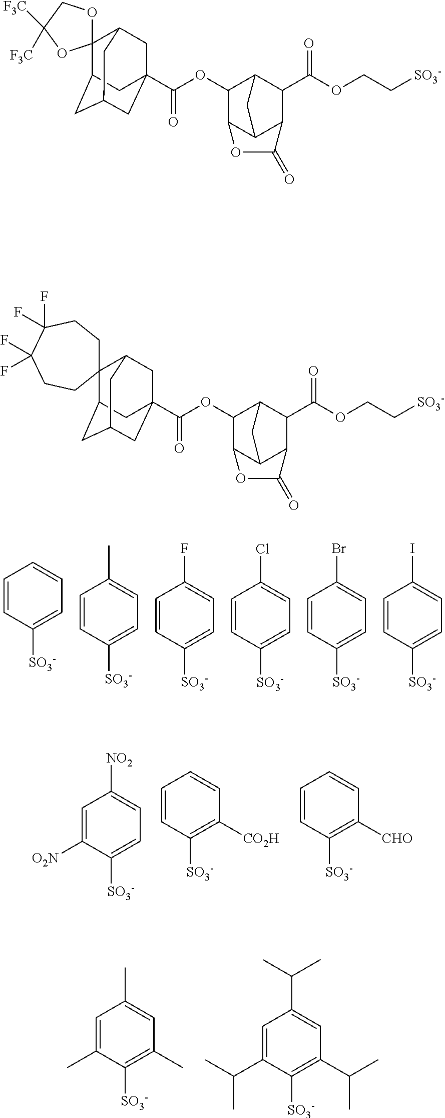

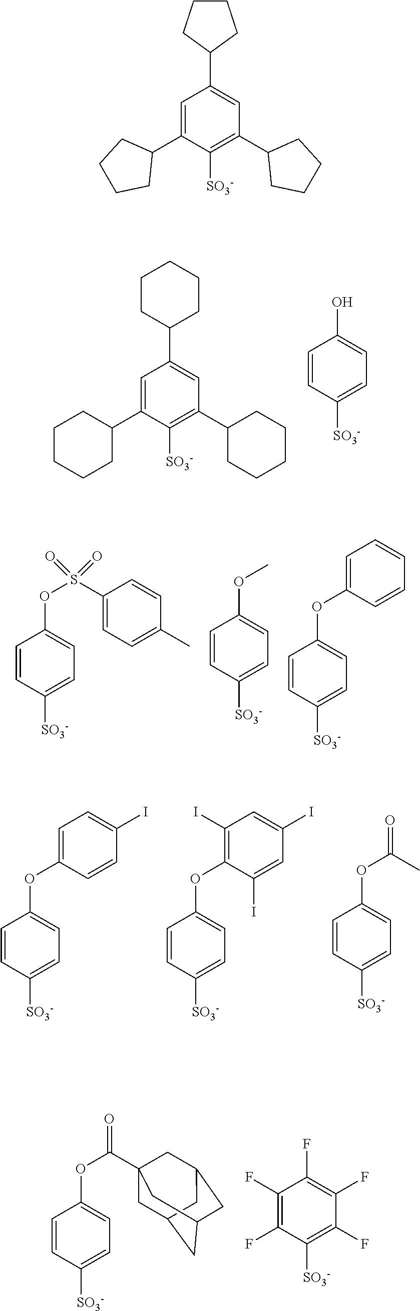

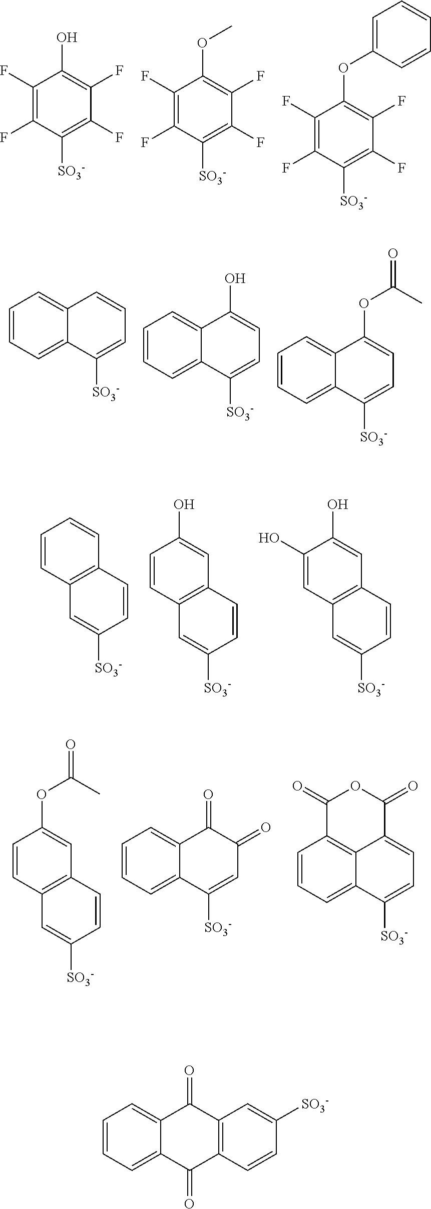

Other specific examples of the anion of the onium salt having the formula (5) include those described in paragraphs [0027] to [0029] of JP 6248882, those described in paragraphs [0029] to [0034] of JP 6648726, those described in paragraphs [0220] to [0225] of JP 7032549, those described in paragraphs [0028] and [0029] of JP 7067271, those described in paragraphs [0037] to [0060] of JP-A 2024-104830, those described in paragraphs [0039] to [0066] of JP-A 2023-177038, those described in paragraphs [0033] to [0093] of JP-A 2024-140135, and those described in paragraphs [0056] to [0059] of WO 2024/241820.

Specific examples of an anion of the onium salt having the formula (6) include, but are not limited to, those listed below.

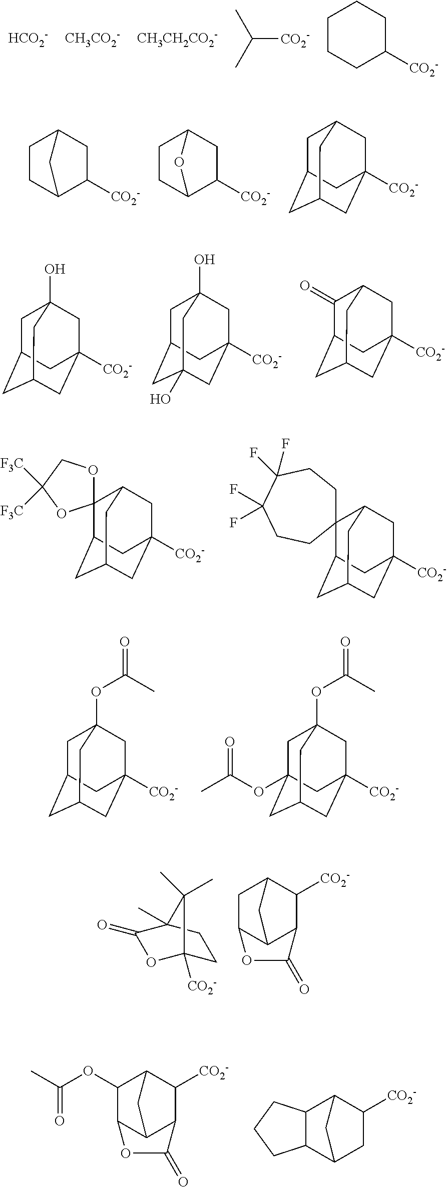

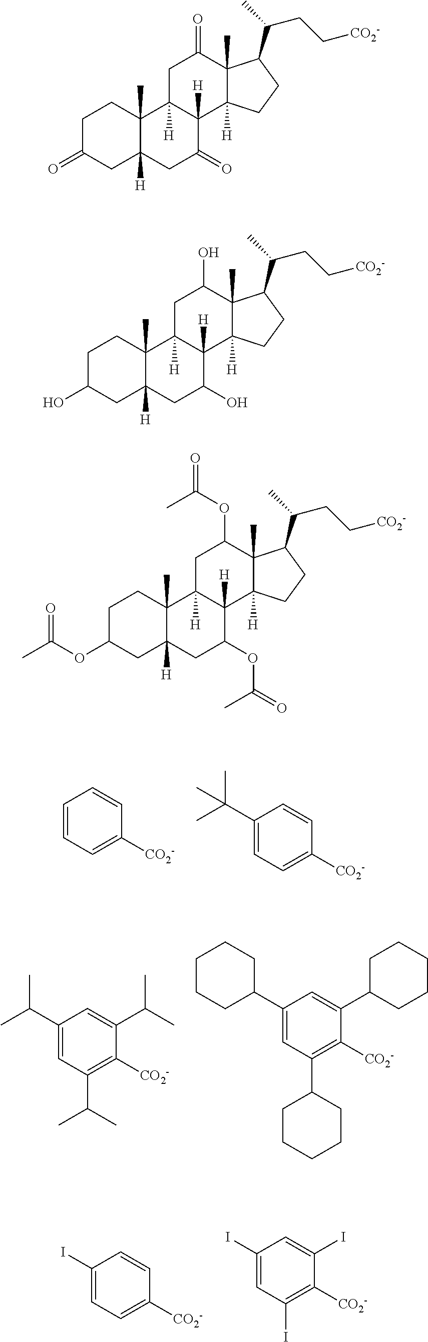

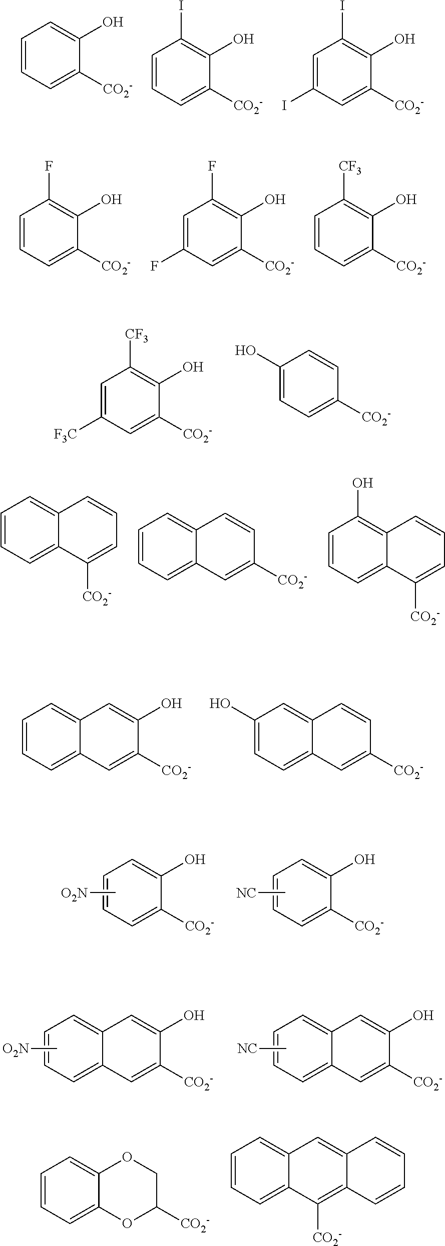

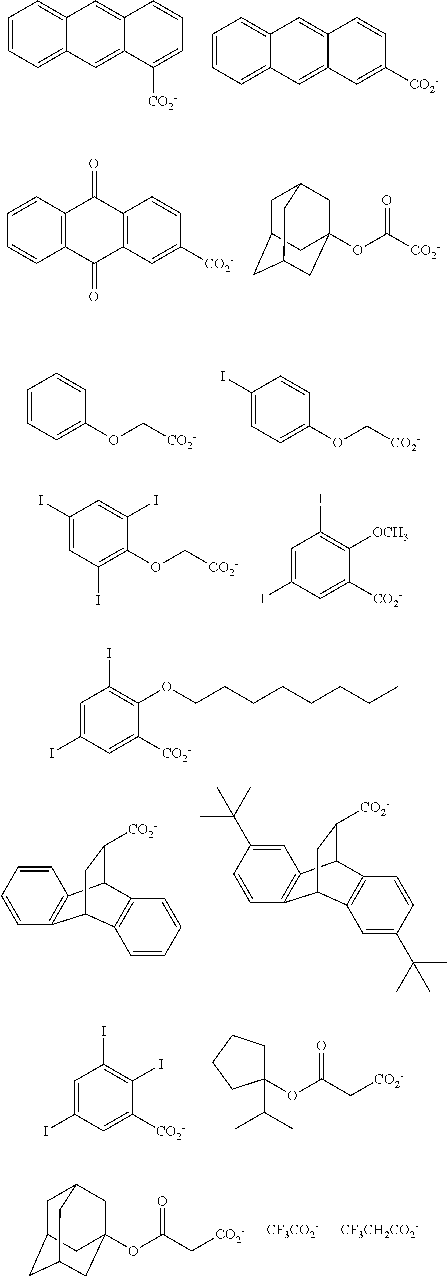

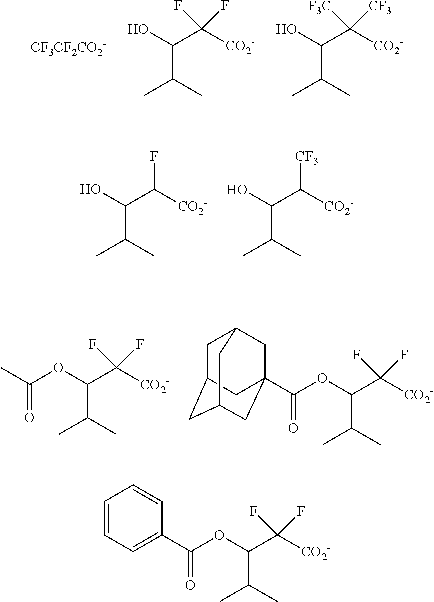

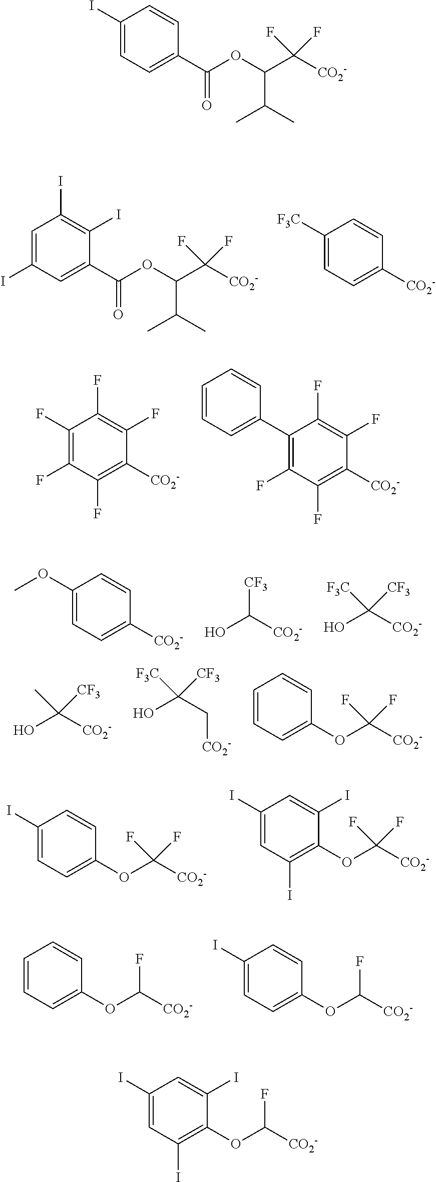

Other specific examples of the anion of the onium salt having the formula (6) include those described in paragraphs [0097] of WO 2023/189502, paragraphs [0231] to [0233] and [0236] of WO 2023/199907, paragraphs [0240] to [0242] of WO 2023/223865, paragraphs [0203] to [0211] of WO 2024/122425, paragraphs [0282] to [0294] of WO 2024/166822, paragraphs [0013] to [0015] of JP-A 2022-077505, paragraphs [0022] to [0024] of JP-A 2022-091720, paragraphs [0026] to [0031] of JP-A 2022-141598, paragraphs [0017] and [0018] of JP-A 2023-018671, paragraph [0157] of JP-A 2023-062898, paragraph [0147] of JP-A 2023-108593, paragraphs [0181] to [0194] of JP-A 2024-040793, paragraph [0171] of JP-A 2024-067732, paragraphs [0029] and [0030] of JP-A 2024-074267, paragraph [0022] of JP-A 2024-074268, paragraphs [0233] to [0237] of JP-A 2024-079160, paragraphs [0025] and [0026] of JP-A 2024-083302, paragraphs [0030] and [0031] of JP-A 2024-083305, paragraphs [0032] to [0034] of JP-A 2024-084141, paragraphs [0030] and [0031] of JP-A 2024-099498, paragraphs [0032] and [0033] of JP-A 2024-099503, paragraphs [0032] and [0033] of JP-A 2024-101558, paragraphs [0027] and [0028] of JP-A 2024-102839, paragraphs [0035] and [0036] of JP-A 2024-102841, paragraphs [0256] to [0261] of JP-A 2024-113453, paragraph [0266] of JP-A 2024-131484, paragraphs [0022] to [0024] of JP-A 2025-010082, paragraph [0299] of JP-A 2025-027484, paragraphs [0190] to [0198] of JP 7611887, paragraphs [0285] to [0290] of JP 7614747, and paragraphs [0201] to [0209] of JP 7621323.

In the formulas (5) and (6), Mq⁺ is an onium cation. Examples of the onium cation include a sulfonium cation, an iodonium cation, and an ammonium cation. Specific examples of the sulfonium cation include those exemplified as specific examples of the cation of the sulfonium salt having the formula (1), those described in paragraphs [0102] to [0125] of JP-A 2024-3744, those described in paragraphs [0044] to [0049] of WO 2024/128017, those described in paragraphs [0035] to [0046] of JP 7491173, and those exemplified as specific examples of the sulfonium cation having the formula (sulfo-1), but are not limited thereto. Specific examples of the iodonium cation include, but are not limited to, those described in paragraph [0181] of JP-A 2024-259. Specific examples of the ammonium cation include those exemplified as specific examples of the ammonium cation having the formula (am-1).

Specific examples of the onium salt having the formula (5) or (6) include any combination of the anion and the cation described above. Note that these onium salts are easily prepared by an ion exchange reaction using a known organic chemical method. For the ion exchange reaction, for example, JP-A 2007-145797 can be referred to.

The onium salt having the formula (5) or (6) acts as a quencher in the chemically amplified resist composition of the present invention. This is because each counter anion of the onium salt is a conjugate base of a weak acid. The weak acid as used herein means an acid having an acidity at which an acid-unstable group of an acid-unstable group-containing unit used for the base polymer cannot perform deprotection. The onium salt having the formula (5) or (6) functions as a quencher when used in combination with an onium salt type photoacid generator having a conjugate base of a strong acid such as a sulfonic acid as a counter anion. That is, in a case where an onium salt that generates a strong acid such as a sulfonic acid and an onium salt that generates a weak acid such as a carboxylic acid are mixed and used, when a strong acid generated from a photoacid generator by irradiation with a high energy ray collides with an unreacted onium salt having a weak acid anion, the weak acid is released by salt exchange to generate an onium salt having a strong acid anion. In this process, the strong acid is exchanged with a weak acid having lower catalytic ability, and therefore the acid is apparently deactivated and acid diffusion can be controlled.

As the other quencher (E), an onium salt having a sulfonium cation and a phenoxide anion moiety in the same molecule described in JP 6848776, an onium salt having a sulfonium cation and a carboxylate anion moiety in the same molecule described in JP 6583136 and JP-A 2020-200311, and an onium salt having an iodonium cation and a carboxylate anion moiety in the same molecule described in JP 6274755 can also be used.

Here, when the photoacid generator that generates a strong acid is an onium salt, as described above, a strong acid generated by irradiation with a high energy ray can be exchanged with a weak acid, but on the other hand, it is considered that it is difficult for a weak acid generated by irradiation with a high energy ray to perform salt exchange by colliding with an unreacted onium salt that generates a strong acid. This is due to a phenomenon that the onium cation easily forms an ion pair with an anion of a stronger acid.

When the chemically amplified resist composition of the present invention contains the onium salt having the formula (5) or (6) as the other quencher (E), the content thereof is preferably 0.1 to 20 parts by weight, and more preferably 0.1 to 10 parts by weight per 80 parts by weight of the base polymer (B). If the content of the onium salt type quencher is in the above range, resolution is favorable, and sensitivity is not significantly lowered, which is preferable. The onium salt having the formula (5) or (6) may be used singly or in combination of two or more types thereof.

The chemically amplified resist composition of the present invention may contain a nitrogen-containing compound as the other quencher (E). Specific examples of the nitrogen-containing compound include primary, secondary, and tertiary amine compounds described in paragraphs [0146] to [0164] of JP-A 2008-111103, particularly an amine compound having a hydroxy group, an ether bond, an ester bond, a lactone ring, a cyano group, or a sulfonate bond. Examples thereof further include a compound obtained by protecting a primary or secondary amine with a carbamate group, described in JP 3790649. Examples thereof further include a compound having a structure in which any of a lactone ring, a sultone ring, a lactam ring, and a sultam ring and an acid-unstable group coexist, described in JP 7615989.

In addition, a sulfonic acid sulfonium salt having a nitrogen-containing substituent may be used as the nitrogen-containing compound. Such a compound functions as a quencher in an unexposed portion, and functions as a so-called photodegradable base that loses quencher ability by neutralization with an acid generated by the compound itself in an exposed portion. By using the photodegradable base, a contrast between the exposed portion and the unexposed portion can be further enhanced. As the photodegradable base, for example, JP-A 2009-109595 and JP-A 2012-46501 can be referred to.

When the chemically amplified resist composition of the present invention contains a nitrogen-containing compound as the other quencher (E), the content thereof is preferably 0.001 to 12 parts by weight, and more preferably 0.01 to 8 parts by weight per 80 parts by weight of the base polymer (B). The nitrogen-containing compound may be used singly or in combination of two or more types thereof.

### Surfactant (F)

The chemically amplified resist composition of the present invention may further contain a surfactant as a component (F). The surfactant (F) is preferably a surfactant insoluble or hardly soluble in water and soluble in an alkaline developer, or a surfactant insoluble or hardly soluble in water and an alkaline developer. As such a surfactant, those described in JP-A 2010-215608 and JP-A 2011-16746 can be referred to.

As the surfactant insoluble or hardly soluble in water and an alkaline developer, among the surfactants described in the above publications, FC-4430 (manufactured by 3M), SURFLON® S-381 (manufactured by AGC Seimi Chemical Co., Ltd.), OLFIN® E1004 (manufactured by Nissin Chemical Industry Co., Ltd.), KH-20 and KH-30 (manufactured by AGC Seimi Chemical Co., Ltd.), an oxetane ring-opened polymer having the following formula (surf-1), and the like are preferable.

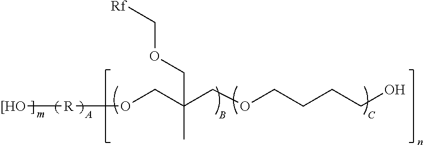

Here, R, Rf, A, B, C, m, n apply only to formula (surf-1) regardless of the above description. R is a di- to tetra-valent C₂-C₅ aliphatic group. Examples of the divalent aliphatic group include an ethylene group, a 1,4-butylene group, a 1,2-propylene group, a 2,2-dimethyl-1,3-propylene group, and a 1,5-pentylene group, and examples of the trivalent or tetravalent aliphatic group include the following groups:

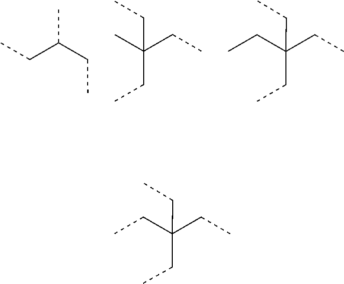

wherein broken lines are bonds, and are partial structures derived from glycerol, trimethylolethane, trimethylolpropane, and pentaerythritol, respectively.

Among these, a 1,4-butylene group, a 2,2-dimethyl-1,3-propylene group, and the like are preferable.

Rf is a trifluoromethyl group or a pentafluoroethyl group, and preferably a trifluoromethyl group. m is an integer of 0 to 3, n is an integer of 1 to 4, and a sum of n and m is a valence of R and an integer of 2 to 4. A is 1. B is an integer of 2 to 25, and preferably an integer of 4 to 20. C is an integer of 0 to 10, and preferably 0 or 1. Constituent units in the formula (surf-1) do not define an arrangement thereof, and may be bonded in a block manner or randomly. Manufacture of a partially fluorinated oxetane ring-opened polymer-based surfactant is described in detail in U.S. Pat. No. 5,650,483 and the like.

When a resist protective film is not used in ArF immersion lithography, a surfactant insoluble or hardly soluble in water and soluble in an alkaline developer has a function of reducing water penetration and leaching by being oriented on a surface of a resist film. Therefore, the surfactant insoluble or hardly soluble in water and soluble in an alkaline developer is useful for suppressing elution of a water-soluble component from the resist film to reduce damage to an exposure apparatus, and is also useful because it is solubilized during alkali aqueous solution development after exposure or after post exposure bake (PEB), and hardly becomes a foreign matter that causes defects. Such a surfactant is a polymeric surfactant having a property of being insoluble or hardly soluble in water and being soluble in an alkaline developer, is also called a hydrophobic resin, and is particularly preferably a surfactant having high water repellency and improving water slippability.

Specific examples of such a polymeric surfactant include those containing at least one selected from a repeat unit having the following formula (7A) (hereinafter, also referred to as a repeat unit 7A), a repeat unit having the following formula (7B) (hereinafter, also referred to as a repeat unit 7B), a repeat unit having the following formula (7C) (hereinafter, also referred to as a repeat unit 7C), a repeat unit having the following formula (7D) (hereinafter, also referred to as a repeat unit 7D), and a repeat unit having the following formula (7E) (hereinafter, also referred to as a repeat unit 7E).

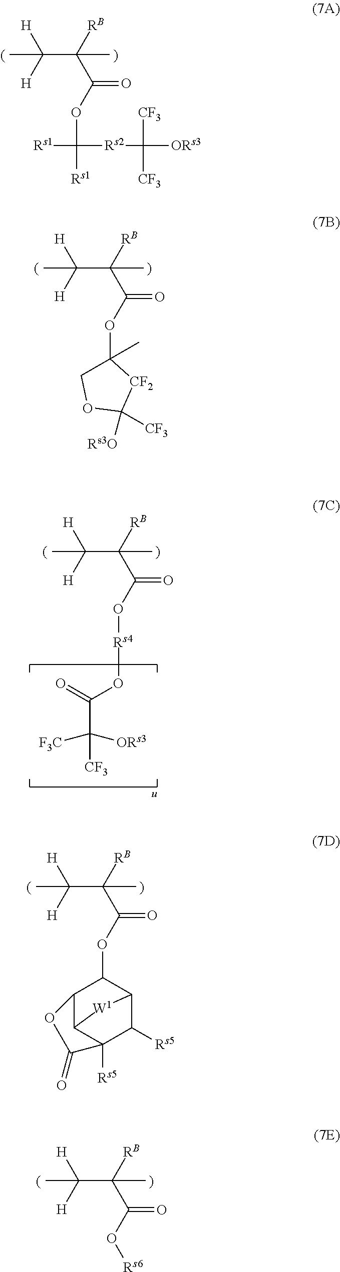

In the formulas (7A) to (7E), R^{B} is a hydrogen atom, a fluorine atom, a methyl group, or a trifluoromethyl group. W¹ is —CH₂—, —CH₂CH₂—, —O—, or two (—H)s separated from each other. R^{s1}s are each independently a hydrogen atom or a C₁-C₁₀ hydrocarbyl group. R^{s2} is a single bond or a linear or branched C₁-C₅ hydrocarbylene group. R^{s3}s are each independently a hydrogen atom, a C₁-C₁₅ hydrocarbyl group or fluorinated hydrocarbyl group, or an acid-unstable group. When R^{s3} is a hydrocarbyl group or a fluorinated hydrocarbyl group, an ether bond or a carbonyl group may be interposed between a carbon-carbon bond. R^{s4} is a (u+1)-valent C₁-C₂₀ hydrocarbon group or fluorinated hydrocarbon group. u is 1, 2, or 3. R^{s5}s are each independently a hydrogen atom or a group of —C(═O)—O—R^{sa}. R^{sa} is a C₁-C₂₀ fluorinated hydrocarbyl group. R^{s6} is a C₁-C₁₅ hydrocarbyl group or fluorinated hydrocarbyl group, and an ether bond or a carbonyl group may be interposed between a carbon-carbon bond thereof.

The C₁-C₁₀ hydrocarbyl group of R^{s1} is preferably a saturated hydrocarbyl group, and may be linear, branched, or cyclic. Specific examples thereof include: a C₁-C₁₀ alkyl group such as a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a n-nonyl group, or a n-decyl group; and a C₃-C₁₀ cyclic saturated hydrocarbyl group such as a cyclopropyl group, a cyclobutyl group, a cyclopentyl group, a cyclohexyl group, an adamantyl group, or a norbornyl group. Among these, groups having 1 to 6 carbon atoms are preferable.

The hydrocarbylene group of R^{s2} is preferably a saturated hydrocarbylene group, and may be linear, branched, or cyclic. Specific examples thereof include a methylene group, an ethylene group, a propylene group, a butylene group, and a pentylene group.

The hydrocarbyl group of R^{s3} or R^{s6} may be saturated or unsaturated, and may be linear, branched, or cyclic. Specific examples thereof include a saturated hydrocarbyl group, an aliphatic unsaturated hydrocarbyl group such as an alkenyl group or an alkynyl group, and a saturated hydrocarbyl group is preferable. Specific examples of the saturated hydrocarbyl group include an undecyl group, a dodecyl group, a tridecyl group, a tetradecyl group, and a pentadecyl group in addition to those exemplified as the hydrocarbyl group of R^{s1}. Examples of the fluorinated hydrocarbyl group of R^{s3} or R^{s6} include groups in which some or all of the hydrogen atoms bonded to carbon atoms of the above-described hydrocarbyl group are replaced with fluorine atoms. As described above, an ether bond or a carbonyl group may be interposed between a carbon-carbon bond of these groups.

Specific examples of the acid-unstable group of R^{s3} include groups having the formulas (AL-3) to (AL-5) described above, a trialkylsilyl group in which each alkyl group is a C₁-C₆ alkyl group, and a C₄-C₂₀ oxo group-containing alkyl group.

The (u+1)-valent hydrocarbon group or fluorinated hydrocarbon group of R^{s4} may be linear, branched, or cyclic, and specific examples thereof include a group obtained by further eliminating u hydrogen atoms from the above-described hydrocarbyl group, fluorinated hydrocarbyl group, or the like.

The fluorinated hydrocarbyl group of R^{sa} is preferably saturated, and may be linear, branched, or cyclic. Specific examples thereof include groups in which some or all of the hydrogen atoms of the hydrocarbyl group are replaced with fluorine atoms, and specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl) ethyl group, a 2-(perfluorohexyl) ethyl group, a 2-(perfluorooctyl) ethyl group, and a 2-(perfluorodecyl) ethyl group.

Specific examples of the repeat units 7A to 7E include, but are not limited to, those listed below. Note that, in the following formulas, R^{B} is as defined above.

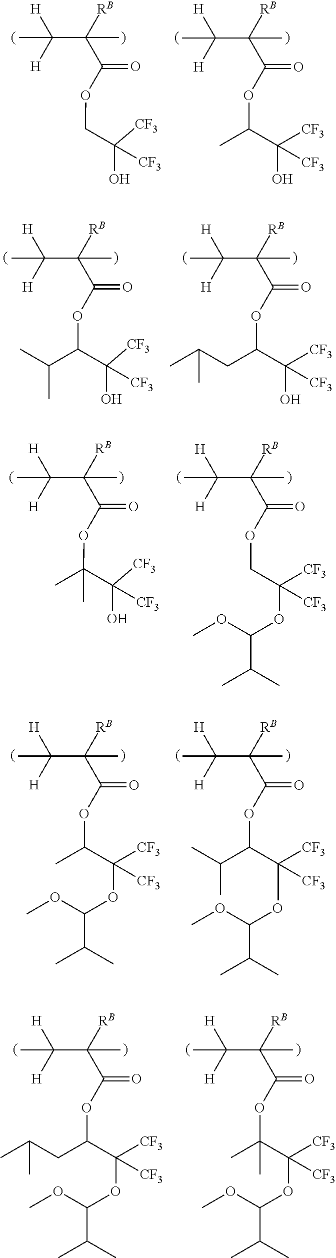

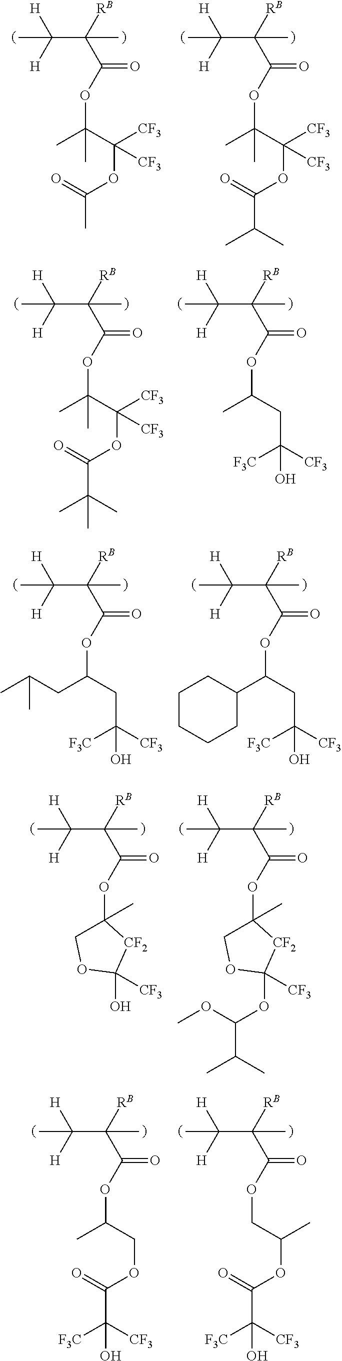

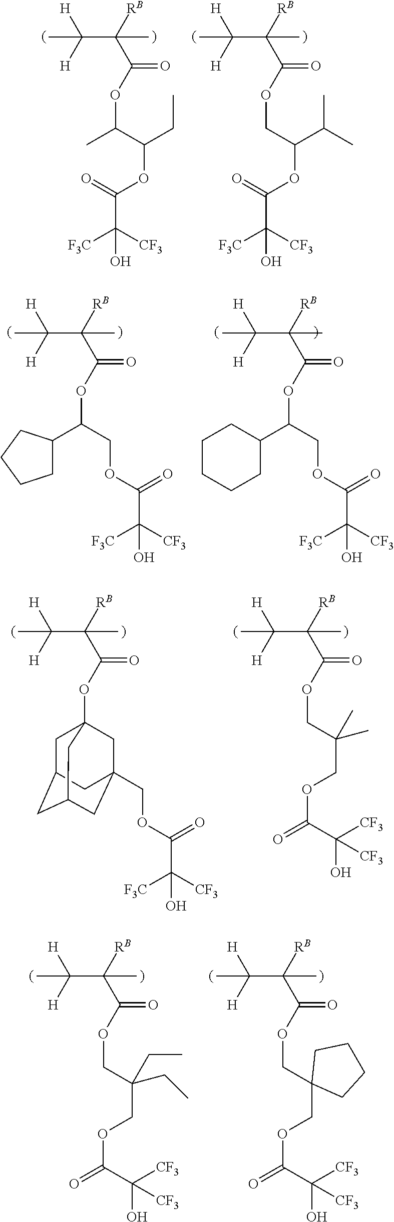

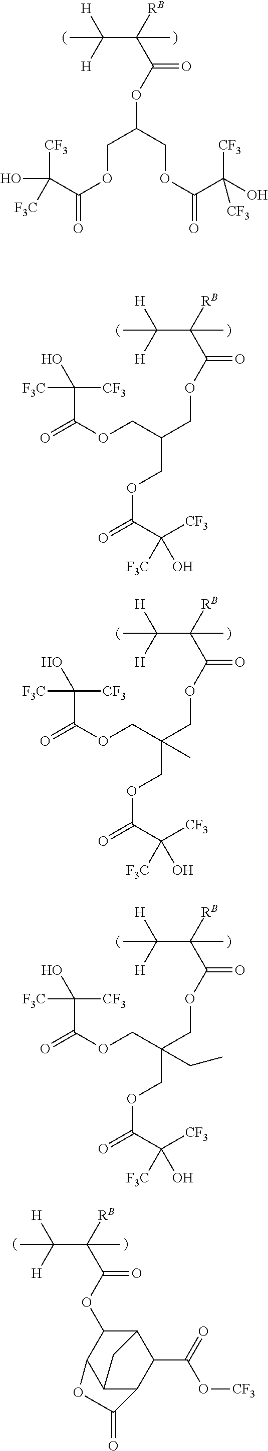

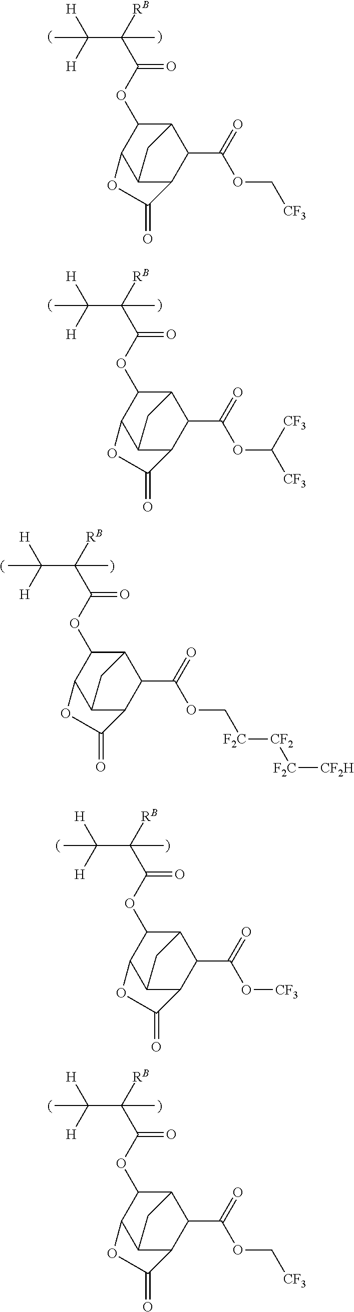

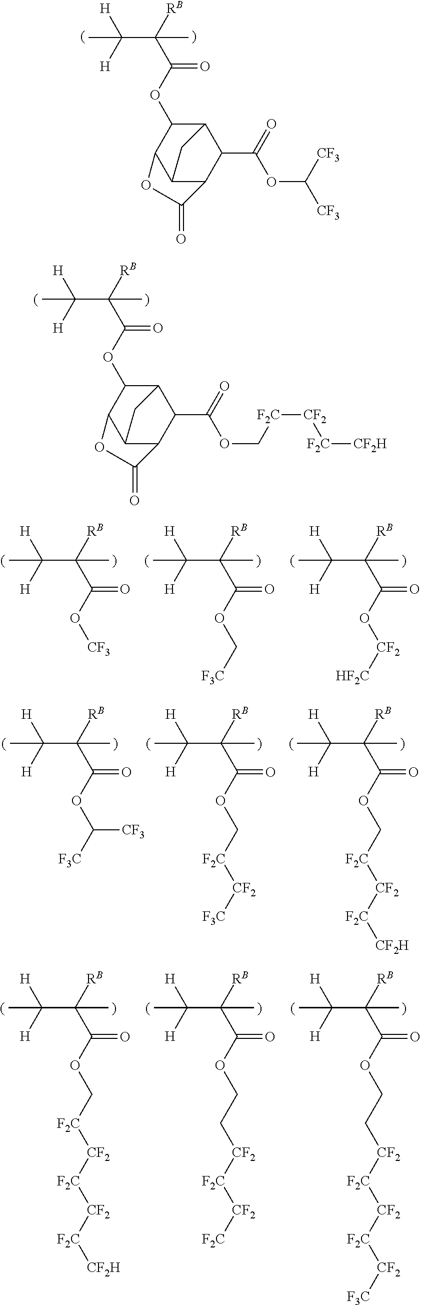

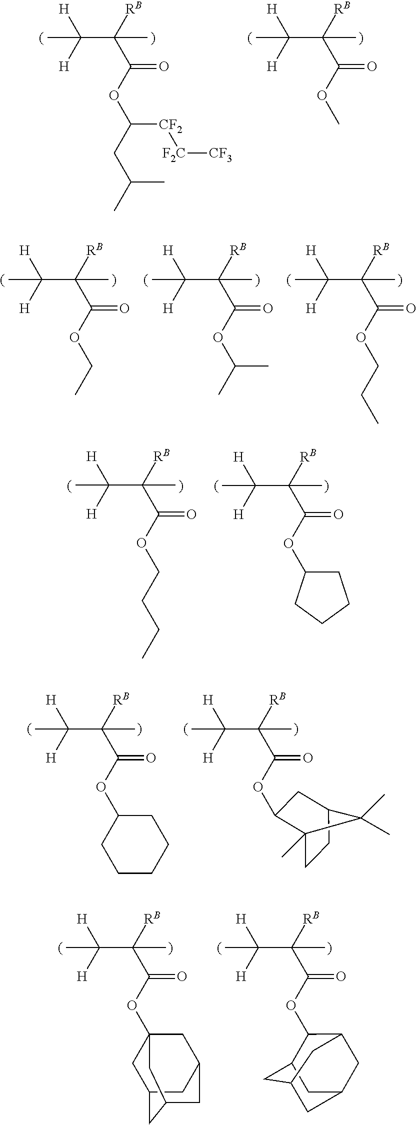

The polymeric surfactant may further contain a repeat unit other than the repeat units 7A to 7E. Specific examples of the other repeat unit include a repeat unit obtained from methacrylic acid, an α-trifluoromethylacrylic acid derivative, or the like. The content of the repeat units 7A to 7E in the polymeric surfactant is preferably 20 mol % or more, more preferably 60 mol % or more, and still more preferably 100 mol % in all repeat units.

Mw of the polymeric surfactant is preferably 1000 to 500,000, and more preferably 3000 to 100,000. Mw/Mn is preferably 1.0 to 2.0, and more preferably 1.0 to 1.6.

Examples of a method for synthesizing the polymeric surfactant include a method in which a monomer containing an unsaturated bond that provides at least one selected from the repeat units 7A to 7E and if necessary, another repeat unit is heated in an organic solvent together with a radical initiator to be polymerized. Specific examples of an organic solvent used during the polymerization include toluene, benzene, THF, diethyl ether, and dioxane. Specific examples of a polymerization initiator include AIBN, 2,2′-azobis(2,4-dimethylvaleronitrile), dimethyl 2,2-azobis(2-methylpropionate), benzoyl peroxide, and lauroyl peroxide. A reaction temperature is preferably 50 to 100° C. A reaction time is preferably 4 to 24 hours. An acid-unstable group introduced into the monomer may be used as it is, or protection or partial protection may be performed after polymerization.

When the polymeric surfactant is synthesized, a known chain transfer agent such as dodecyl mercaptan or 2-mercaptoethanol may be used in order to adjust a molecular weight. In this case, the addition amount of these chain transfer agents is preferably 0.01 to 10 mol % based on the total moles of monomers to be polymerized.

When the chemically amplified resist composition of the present invention contains the surfactant (F), the content thereof is preferably 0.1 to 50 parts by weight, and more preferably 0.5 to 10 parts by weight, per 80 parts by weight of the base polymer (B). If the content of the surfactant (F) is 0.1 parts by weight or more, a receding contact angle between a surface of a resist film and water is sufficiently improved. If the content of the surfactant (F) is 50 parts by weight or less, a dissolution rate of the surface of the resist film in a developer is low, and the height of a formed fine pattern is sufficiently maintained. The surfactant (F) may be used singly or in combination of two or more types thereof.

### Other Components (G)

The chemically amplified resist composition of the present invention may contain, as other components (G), a compound that is decomposed by an acid to generate an acid (acid increase compound), an organic acid derivative, a fluorine-substituted alcohol, a compound (dissolution inhibitor) that has Mw of 3000 or less and changes its solubility in a developer by an action of an acid, and the like. As the acid increase compound, a compound described in JP-A 2009-269953 or JP-A 2010-215608 can be referred to. When the chemically amplified resist composition of the present invention contains the acid increase compound, the content thereof is preferably 0 to 5 parts by weight, and more preferably 0 to 3 parts by weight, per 80 parts by weight of the base polymer (B). When the content is too large, it is difficult to control acid diffusion, and resolution and a pattern shape may be deteriorated. As the organic acid derivative, the fluorine-substituted alcohol, and the dissolution inhibitor, a compound described in JP-A 2009-269953 or JP-A 2010-215608 can be referred to.

### [Pattern Forming Method]

A pattern forming method of the present invention includes: a step of forming a resist film on a substrate using the above-described chemically amplified resist composition; a step of exposing the resist film to a high energy ray; and a step of developing the exposed resist film using a developer.

As the substrate, for example, a substrate for manufacturing an integrated circuit (Si, SiO₂, SiN, SiON, TiN, WSi, BPSG, SOG, an organic antireflection film, or the like) or a substrate for manufacturing a mask circuit (Cr, CrO, CrON, MoSi₂, SiO₂, or the like) can be used.

The resist film can be formed by, for example, applying the chemically amplified resist composition onto a substrate by a method such as spin coating so as to have a film thickness of preferably 0.05 to 2 μm, and prebaking the chemically amplified resist composition on a hot plate preferably at 60 to 150° C. for one to ten minutes, more preferably at 80 to 140° C. for one to five minutes.

Examples of the high energy ray used for exposure of the resist film include a KrF excimer laser beam, an ArF excimer laser beam, EB, and EUV having a wavelength of 3 to 15 nm. When a KrF excimer laser beam, an ArF excimer laser beam, or EUV is used, exposure can be performed by using a mask for forming a target pattern and performing irradiation such that an exposure dose is preferably 1 to 200 mJ/cm² and more preferably 10 to 100 mJ/cm². When EB is used, irradiation is performed using a mask for forming a target pattern or directly such that an exposure dose is preferably 1 to 300 μC/cm² and more preferably 10 to 200 μC/cm².

Note that, in addition to a normal exposure method, it is also possible to use an immersion method in which exposure is performed by interposing a liquid having a refractive index of 1.0 or more between a resist film and a projection lens. In this case, it is also possible to use a water-insoluble protective film.

The water-insoluble protective film is used to prevent an eluate from the resist film and to increase water slippability of a film surface, and there are roughly two types of water-insoluble protective films. One is an organic solvent peeling type in which peeling is required before alkali aqueous solution development by an organic solvent that does not dissolve a resist film, and the other is an alkali aqueous solution soluble type which is soluble in an alkaline developer and removes a protective film together with removal of a resist film soluble portion. The latter is particularly preferably a material based on a polymer having a 1,1,1,3,3,3-hexafluoro-2-propanol residue insoluble in water and soluble in an alkaline developer, and dissolved in an alcohol-based solvent having 4 or more carbon atoms, an ether-based solvent having 8 to 12 carbon atoms, or a mixed solvent thereof. The above-described surfactant insoluble in water and soluble in an alkaline developer can be a material dissolved in an alcohol-based solvent having 4 or more carbon atoms, an ether-based solvent having 8 to 12 carbon atoms, or a mixed solvent thereof.

PEB may be performed after the exposure. PEB can be performed, for example, by performing heating on a hot plate preferably at 60 to 150° C. for one to five minutes, more preferably at 80 to 140° C. for one to three minutes.

As for the development, for example, by performing development using a developer of an alkali aqueous solution such as tetramethylammonium hydroxide (TMAH) of preferably 0.1 to 5 wt %, more preferably 2 to 3 wt % preferably for 0.1 to three minutes, more preferably for 0.5 to two minutes by a conventional method such as a dip method, a puddle method, or a spray method, an exposed portion is dissolved, and a target pattern is formed on a substrate.

After the resist film is formed, the acid generator and the like may be extracted from a film surface by performing pure water rinsing, particles may be washed off, or rinsing for removing water remaining on the film after the exposure may be performed.

Furthermore, pattern formation may be performed by a double patterning method. Examples of the double patterning method include a trench method in which an underlayer of a 1:3 trench pattern is processed by first exposure and etching, and a 1:3 trench pattern is formed by second exposure while shifting the position, thereby forming a 1:1 pattern, and a line method in which a first underlayer of a 1:3 isolation-remaining pattern is processed by first exposure and etching, and a second underlayer in which the 1:3 isolation-remaining pattern is formed under the first underlayer is processed by second exposure while shifting the position, thereby forming a 1:1 pattern with a half pitch.

In the pattern forming method of the present invention, a method of negative tone development in which an unexposed portion is dissolved using an organic solvent as a developer instead of the alkaline aqueous solution may be used.

In the organic solvent development, as the developer, 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, 2-phenylethyl acetate, and the like can be used. These organic solvents may be used singly or in combination of two or more types thereof.

### EXAMPLES

Hereinafter, the present invention is described specifically with reference to Synthesis Examples, Examples, and Comparative Examples, but the present invention is not limited to the following Examples. Note that a device used is as follows.

MALDI TOF-MS: S3000 manufactured by JEOL Ltd.

### [1] Synthesis of Sulfonium Salt

### Example 1-1

### Synthesis of Sulfonium Salt PDQ-1

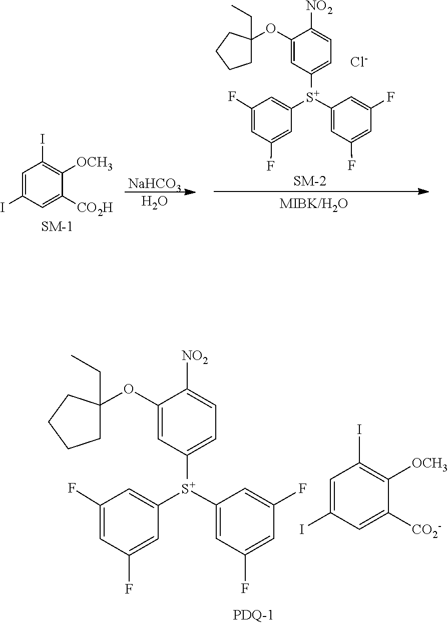

### (1) Synthesis of Sulfonium Salt PDQ-1

In a nitrogen atmosphere, a compound SM-1 (4.0 g), sodium hydrogen carbonate (1.0 g) and water (20 g) were mixed in a reaction vessel, and the mixture was stirred at an internal temperature of 45° C. for one hour. Thereafter, the reaction liquid was cooled to room temperature, a raw material SM-2 (5.3 g) and methyl isobutyl ketone (30 g) were added thereto, and the mixture was stirred at room temperature for 30 minutes. After stirring, the organic layer was separated, washed with water, and then concentrated under reduced pressure. The concentrate was purified by silica gel chromatography to obtain 8.3 g of the sulfonium salt PDQ-1 as an objective product as an oily product (yield: 93%).

Results of TOF-MS for PDQ-1 are described below.

### MALDI TOF-MS:

POSITIVE M⁺ 492 (corresponding to C₂₅H₂₂F₄NO₃S⁺)
NEGATIVE M⁻403 (corresponding to C₈H₅I₂O₃⁻)

### Examples 1-2 to 1-7

### Synthesis of Sulfonium Salts PDQ-2 to PDQ-7

Sulfonium salts PDQ-2 to PDQ-7 having the following formulas were synthesized using corresponding raw materials and known organic synthesis reactions.

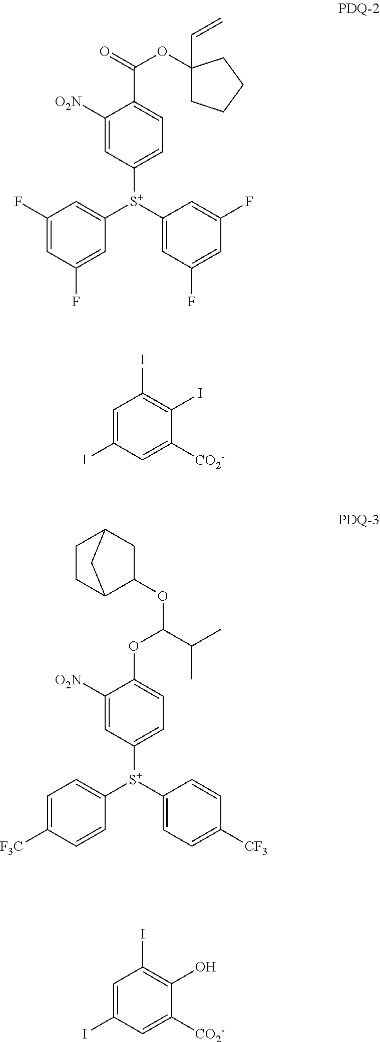

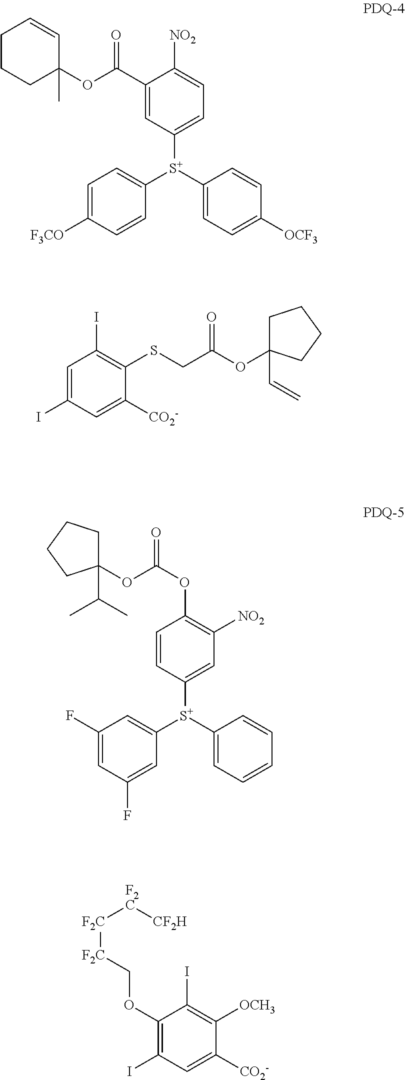

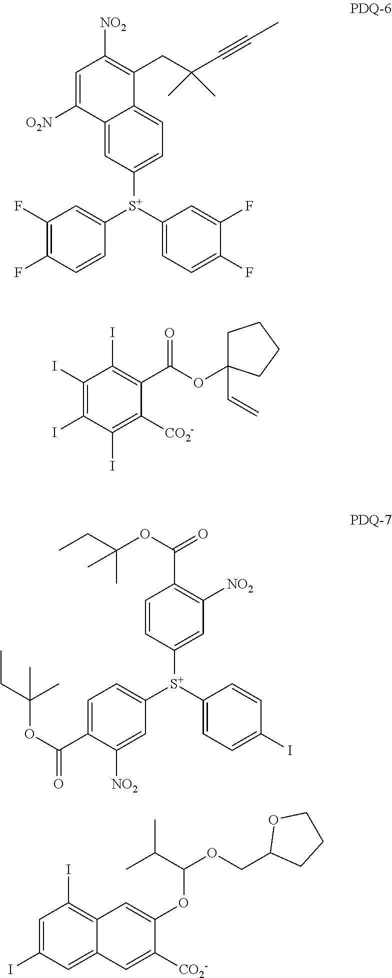

### [2] Synthesis of Base Polymer

### SYNTHESIS EXAMPLE

### Synthesis of Base Polymer (Polymers P-1 to P-5)

Monomers were combined and a copolymerization reaction was performed in MEK as a solvent, the reaction solution was put into hexane, and the precipitated solid was washed with hexane, then isolated, and dried to obtain a base polymer (P-1 to P-5) having the following composition. The composition of the obtained base polymer was confirmed by ¹H-NMR, and Mw and Mw/Mn were confirmed by GPC (solvent: DMF, standard: polystyrene).

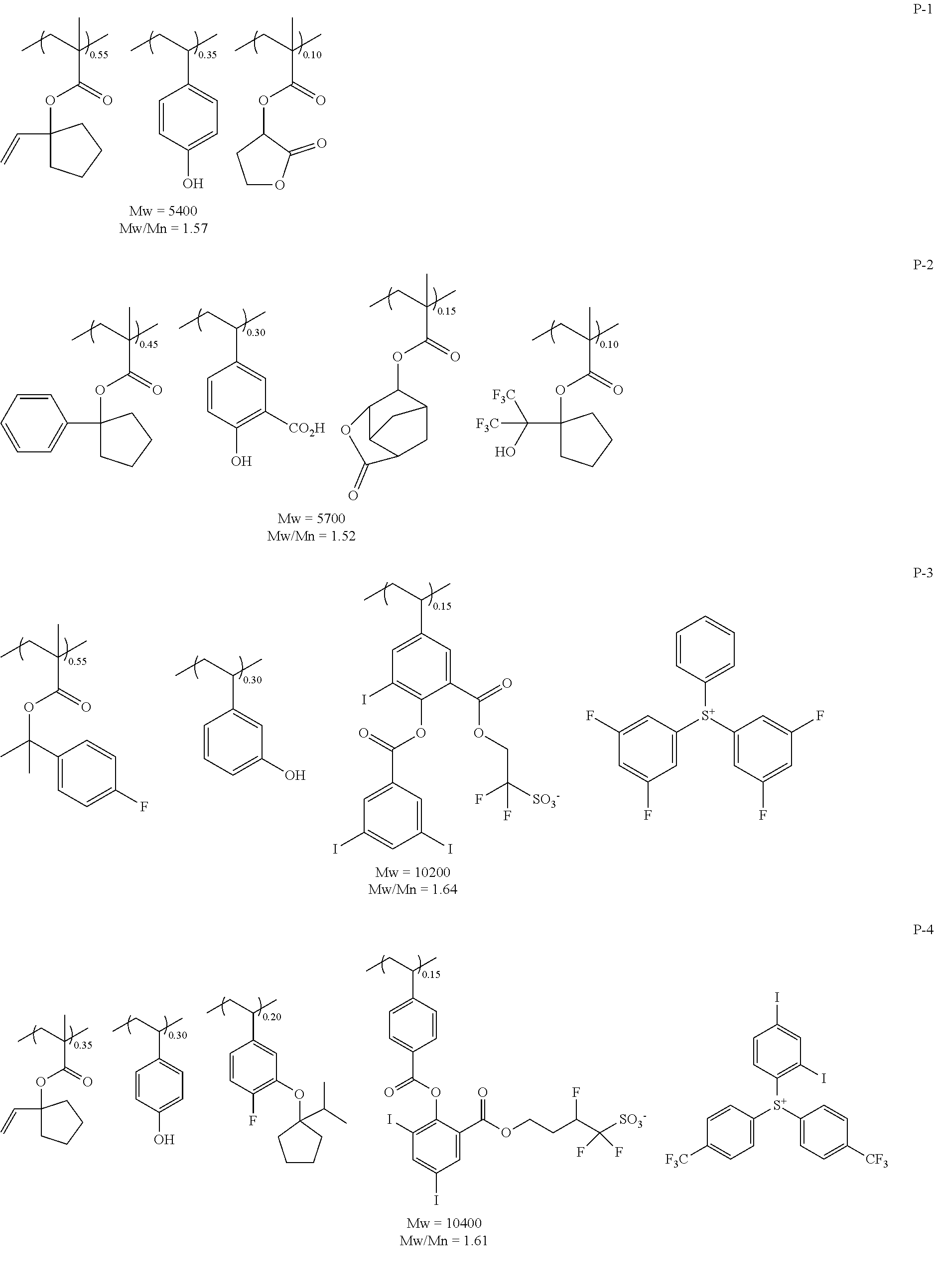

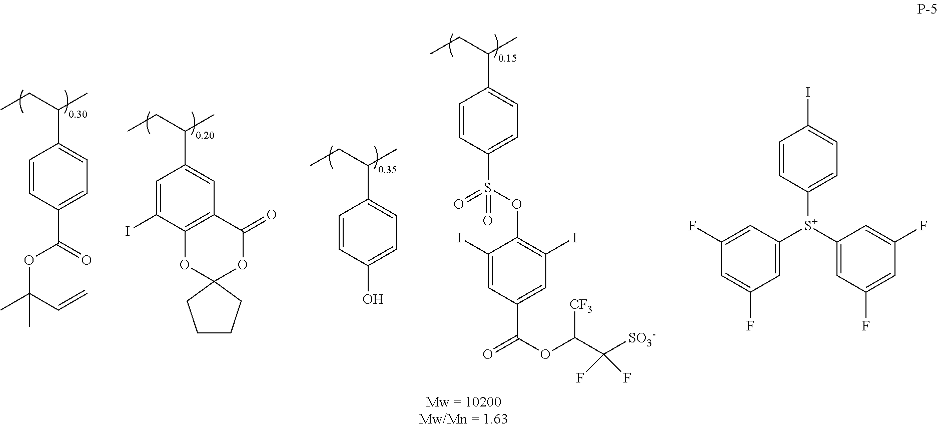

### [3] Preparation of Chemically Amplified Resist Composition

### Examples 2-1 to 2-30 and Comparative Examples 1-1 to 1-20

The quencher of the present invention (PDQ-1 to PDQ-7), a comparative quencher (PDQ-A to PDQ-D), the photoacid generator (PAG-X, PAG-Y, and PAG-Z), the base polymer (P-1 to P-5), and another quencher (AQ-1 and AQ-2) were dissolved in a solvent containing 0.01 wt % of a surfactant A (OMNOVA) with a composition presented in Tables 1 and 2 below to prepare a solution, and the solution was filtered through a 0.2 μm Teflon® filter to prepare a chemically amplified resist composition (R-1 to R-30 and CR-1 to CR-20).

TABLE 1

| | | Resist | Base | | Photoacid | Other | | | |
|---|---|---|---|---|---|---|---|---|---|
| | | compo- | polymer | Quencher | generator | quencher | Solvent 1 | Solvent 2 | Solvent 3 |
| | | sition | (pbw) | (pbw) | (pbw) | (pbw) | (pbw) | (pbw) | (pbw) |
| Example | 2-1 | R-1 | P-1 (80) | PDQ-1 (5) | PAG-X (15) | — | PGMEA (2250) | EL (2800) | DAA (550) |
| | 2-2 | R-2 | P-1 (80) | PDQ-2 (5) | PAG-X (15) | — | PGMEA (2250) | EL (2800) | DAA (550) |
| | 2-3 | R-3 | P-1 (80) | PDQ-3 (5) | PAG-X (15) | — | PGMEA (2250) | EL (2800) | DAA (550) |
| | 2-4 | R-4 | P-1 (80) | PDQ-4 (5) | PAG-X (15) | — | PGMEA (2250) | EL (2800) | DAA (550) |
| | 2-5 | R-5 | P-1 (80) | PDQ-5 (5) | PAG-X (15) | — | PGMEA (2250) | EL (2800) | DAA (550) |
| | 2-6 | R-6 | P-1 (80) | PDQ-6 (5) | PAG-X (15) | — | PGMEA (2250) | EL (2800) | DAA (550) |
| | 2-7 | R-7 | P-1 (80) | PDQ-7 (5) | PAG-X (15) | — | PGMEA (2250) | EL (2800) | DAA (550) |
| | 2-8 | R-8 | P-1 (80) | PDQ-1 (5) | PAG-Y (15) | — | PGMEA (2250) | EL (2800) | DAA (550) |
| | 2-9 | R-9 | P-1 (80) | PDQ-1 (2.5) | PAG-X (15) | AQ-1 (2.5) | PGMEA (2250) | EL (2800) | DAA (550) |
| | 2-10 | R-10 | P-1 (80) | PDQ-2 (3) | PAG-Y (15) | AQ-2 (2) | PGMEA (2250) | EL (2800) | DAA (550) |
| | 2-11 | R-11 | P-2 (80) | PDQ-1 (5) | PAG-X (14) | — | PGMEA (2250) | EL (2800) | DAA (550) |
| | 2-12 | R-12 | P-2 (80) | PDQ-2 (5) | PAG-X (15) | — | PGMEA (2250) | EL (2800) | DAA (550) |
| | 2-13 | R-13 | P-2 (80) | PDQ-4 (5) | PAG-Y (14) | — | PGMEA (2250) | EL (2800) | DAA (550) |
| | 2-14 | R-14 | P-2 (80) | PDQ-6 (5) | PAG-Y (15) | — | PGMEA (2250) | EL (2800) | DAA (550) |
| | 2-15 | R-15 | P-2 (80) | PDQ-1 (4) | PAG-X (10) | AQ-1 (2) | PGMEA (2250) | EL (2800) | DAA (550) |
| | | | | | PAG-Z (5) | | | | |
| | 2-16 | R-16 | P-3 (80) | PDQ-1 (5) | — | — | PGMEA (2250) | EL (2800) | DAA (550) |
| | 2-17 | R-17 | P-3 (80) | PDQ-2 (5) | — | — | PGMEA (2250) | EL (2800) | DAA (550) |
| | 2-18 | R-18 | P-3 (80) | PDQ-3 (5) | — | — | PGMEA (2250) | EL (2800) | DAA (550) |
| | 2-19 | R-19 | P-3 (80) | PDQ-4 (5) | PAG-X (5) | — | PGMEA (2250) | EL (2800) | DAA (550) |
| | 2-20 | R-20 | P-3 (80) | PDQ-2 (3) | — | AQ-1 (2) | PGMEA (2250) | EL (2800) | DAA (550) |
| | 2-21 | R-21 | P-4 (80) | PDQ-1 (5) | — | — | PGMEA (2250) | EL (2800) | DAA (550) |
| | 2-22 | R-22 | P-4 (80) | PDQ-2 (5) | — | — | PGMEA (2250) | EL (2800) | DAA (550) |
| | 2-23 | R-23 | P-4 (80) | PDQ-5 (5) | — | — | PGMEA (2250) | EL (2800) | DAA (550) |
| | 2-24 | R-24 | P-4 (80) | PDQ-6 (6) | PAG-Y (4) | — | PGMEA (2250) | EL (2800) | DAA (550) |
| | | | | | PAG-Z (2) | | | | |
| | 2-25 | R-25 | P-4 (80) | PDQ-2 (5) | — | — | PGMEA (2250) | EL (2800) | DAA (550) |
| | 2-26 | R-26 | P-5 (80) | PDQ-1 (5) | — | — | PGMEA (2250) | EL (2800) | DAA (550) |
| | 2-27 | R-27 | P-5 (80) | PDQ-2 (5) | — | — | PGMEA (2250) | EL (2800) | DAA (550) |
| | 2-28 | R-28 | P-5 (80) | PDQ-7 (3) | PAG-Y (5) | AQ-2 (2) | PGMEA (2250) | EL (2800) | DAA (550) |
| | 2-29 | R-29 | P-5 (80) | PDQ-3 (5) | — | — | PGMEA (2250) | EL (2800) | DAA (550) |
| | 2-30 | R-30 | P-5 (80) | PDQ-2 (2.5) | — | AQ-1 (2.5) | PGMEA (2250) | EL (2800) | DAA (550) |

TABLE 2

| | | | | Compar- | | | | | |
|---|---|---|---|---|---|---|---|---|---|
| | | Resist | Base | ative | Photoacid | Other | | | |
| | | compo- | polymer | quencher | generator | quencher | Solvent 1 | Solvent 2 | Solvent 3 |
| | | sition | (pbw) | (pbw) | (pbw) | (pbw) | (pbw) | (pbw) | (pbw) |
| Comparative | 1-1 | CR-1 | P-1 (80) | PDQ-A (5) | PAG-X (15) | — | PGMEA (2250) | EL (2800) | DAA (550) |
| Example | 1-2 | CR-2 | P-1 (80) | PDQ-B (5) | PAG-X (15) | — | PGMEA (2250) | EL (2800) | DAA (550) |
| | 1-3 | CR-3 | P-1 (80) | PDQ-C (5) | PAG-X (15) | — | PGMEA (2250) | EL (2800) | DAA (550) |
| | 1-4 | CR-4 | P-1 (80) | PDQ-D (5) | PAG-X (15) | — | PGMEA (2250) | EL (2800) | DAA (550) |
| | 1-5 | CR-5 | P-1 (80) | PDQ-A (5) | PAG-Y (15) | — | PGMEA (2250) | EL (2800) | DAA (550) |
| | 1-6 | CR-6 | P-1 (80) | PDQ-B (5) | PAG-Y (15) | — | PGMEA (2250) | EL (2800) | DAA (550) |
| | 1-7 | CR-7 | P-1 (80) | PDQ-C (2.5) | PAG-X (15) | AQ-1 (2.5) | PGMEA (2250) | EL (2800) | DAA (550) |
| | 1-8 | CR-8 | P-1 (80) | PDQ-D (3) | PAG-Y (15) | AQ-2 (2) | PGMEA (2250) | EL (2800) | DAA (550) |
| | 1-9 | CR-9 | P-2 (80) | PDQ-A (5) | PAG-X (15) | — | PGMEA (2250) | EL (2800) | DAA (550) |
| | 1-10 | CR-10 | P-2 (80) | PDQ-B (5) | PAG-X (15) | — | PGMEA (2250) | EL (2800) | DAA (550) |
| | 1-11 | CR-11 | P-2 (80) | PDQ-C (4) | PAG-X (10) | AQ-1 (2) | PGMEA (2250) | EL (2800) | DAA (550) |
| | | | | | PAG-Z (5) | | | | |
| | 1-12 | CR-12 | P-3 (80) | PDQ-A (5) | — | — | PGMEA (2250) | EL (2800) | DAA (550) |
| | 1-13 | CR-13 | P-3 (80) | PDQ-C (5) | — | — | PGMEA (2250) | EL (2800) | DAA (550) |
| | 1-14 | CR-14 | P-3 (80) | PDQ-D (3) | — | AQ-1 (2) | PGMEA (2250) | EL (2800) | DAA (550) |
| | 1-15 | CR-15 | P-4 (80) | PDQ-A (5) | — | — | PGMEA (2250) | EL (2800) | DAA (550) |
| | 1-1 | CR-16 | P-4 (80) | PDQ-D (5) | — | — | PGMEA (2250) | EL (2800) | DAA (550) |
| | 1-17 | CR-17 | P-4 (80) | PDQ-C (6) | PAG-Y (4) | — | PGMEA (2250) | EL (2800) | DAA (550) |
| | | | | | PAG-Z (2) | | | | |
| | 1-18 | CR-18 | P-5 (80) | PDQ-A (5) | — | — | PGMEA (2250) | EL (2800) | DAA (550) |
| | 1-19 | CR-19 | P-5 (80) | PDQ-B (3) | PAG-Y (5) | AQ-2 (2) | PGMEA (2250) | EL (2800) | DAA (550) |
| | 1-20 | CR-20 | P-5 (80) | PDQ-C (5) | — | — | PGMEA (2250) | EL (2800) | DAA (550) |

In Tables 1 and 2, the solvent, the photoacid generators PAG-X, PAG-Y, and PAG-Z, the comparative quenchers PDQ-A to PDQ-D, the other quenchers AQ-1 and AQ-2, and the surfactant A are as follows.

### Solvent:

PGMEA (propylene glycol monomethyl ether acetate)
EL (ethyl lactate)
DAA (diacetone alcohol)

### Photoacid Generators: PAG-X, PAG-Y, and PAG-Z

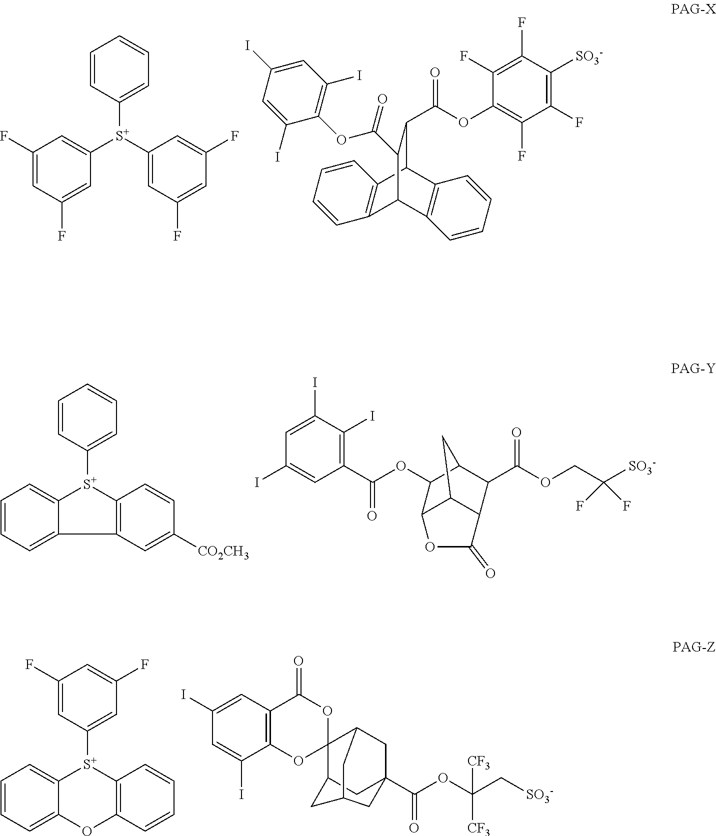

### Comparative Quenchers: PDQ-A to PDQ-D

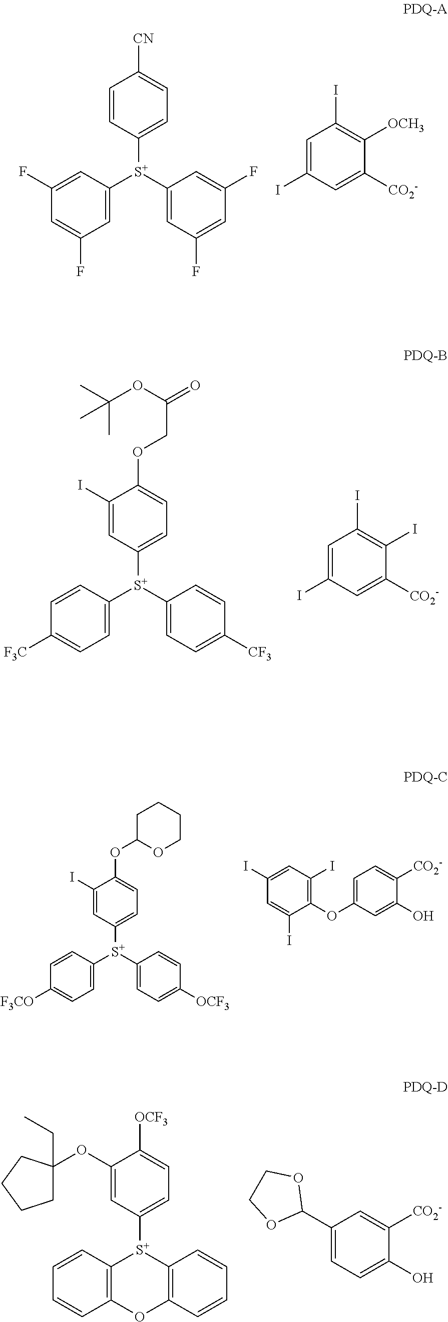

### Other Quenchers: AQ-1 and AQ-2

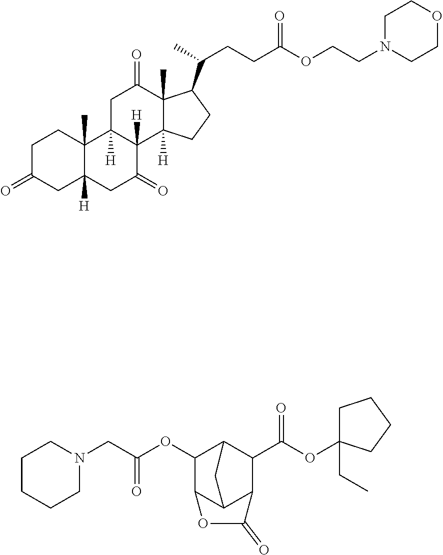

### Surfactant A:

3-methyl-3-(2,2,2-trifluoroethoxymethyl) oxetane/tetrahydrofuran/
2,2-dimethyl-1,3-propanediol copolymer (manufactured by OMNOVA)

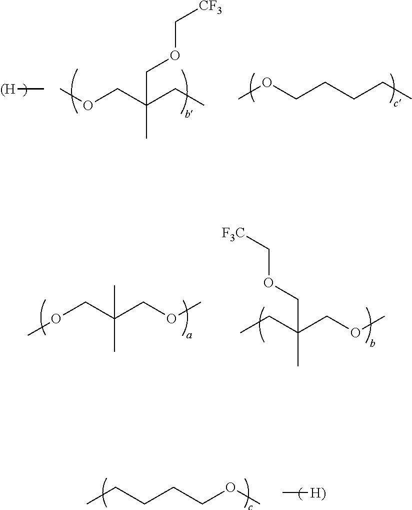

a:(b+b′):(c+c′)=1:4 to 7:0.01 to 1 (molar ratio)
Mw=1,500

### [4] EUV Lithography Evaluation (1)

### Examples 3-1 to 3-30 and Comparative Examples 2-1 to 2-20

Each of the chemically amplified resist compositions (R-1 to R-30 and CR-1 to CR-20) was spin-coated on a Si substrate formed with a silicon-containing spin-on hard mask SHB-A940 (silicon content: 43 wt %) manufactured by Shin-Etsu Chemical Co., Ltd. in a film thickness of 20 nm, and prebaked at 100° C. for 60 seconds using a hot plate to prepare a resist film having a film thickness of 50 nm. The resist film was exposed to light so as to provide an LS pattern having an on-wafer dimension of 18 nm and a pitch of 36 nm with an EUV scanner NXE3400 (NA: 0.33, σ: 0.9/0.6, dipole illumination) manufactured by ASML while an exposure dose and a focus were changed (exposure dose pitch: 1 mJ/cm², focus pitch: 0.020 μm), and after the exposure, PEB was performed at temperatures presented in Tables 3 and 4 for 60 seconds. Thereafter, puddle development was performed with a 2.38 wt % TMAH aqueous solution for 30 seconds, rinsing was performed with a surfactant-containing rinse material, and spin-drying was performed to obtain a positive pattern.

The obtained LS pattern was observed with a length measuring SEM (CG6300) manufactured by Hitachi High-Tech Corporation, and sensitivity, EL, LWR, focal depth (DOF), and a collapse limit were evaluated according to the following methods. In addition, development defect evaluation of the obtained LS pattern was performed. Results thereof are presented in Tables 3 and 4.

### [Sensitivity Evaluation]

An optimum exposure dose Eₒₚ (mJ/cm²) at which an LS pattern having a line width of 18 nm and a pitch of 36 nm could be obtained was determined and taken as sensitivity. The smaller this value is, the higher the sensitivity is.

### [EL Evaluation]

EL (unit:%) was determined from an exposure dose formed within ±10% (16.2 to 19.8 nm) of the space width of 18 nm in the LS pattern by the following formula. The larger this value is, the better the performance is.

$$EL ( \% ) = ( \vert {E}_{1} - {E}_{2} \vert / Eop ) \times 100$$

E₁: Optimum exposure dose that provides an LS pattern having a line width of 16.2 nm and a pitch of 36 nm
E₂: Optimum exposure dose that provides an LS pattern having a line width of 19.8 nm and a pitch of 36 nm
Eₒₚ: Optimum exposure dose that provides an LS pattern having a line width of 18 nm and a pitch of 36 nm

### [LWR Evaluation]

In the LS pattern obtained by irradiation with Eₒₚ, the dimensions of ten points in a longitudinal direction of the line were measured, and from the result, a value (3σ) three times a standard deviation (σ) was obtained as LWR. As this value is smaller, a pattern having a smaller roughness and a more uniform line width can be obtained.

### [DOF Evaluation]

As focus depth evaluation, a focus range formed in a range of ±10% (16.2 to 19.8 nm) of a dimension of 18 nm in the LS pattern was obtained. The larger this value is, the wider focus depth is.

### [Evaluation of Line Pattern Collapse Limit]

The line dimensions of ten points in a longitudinal direction were measured at each exposure dose in an optimal focus of the LS pattern. The thinnest line dimension obtained without collapse was defined as a collapse limit dimension. The smaller this value is, the better a collapse limit is.

### [Development Defect Evaluation]

In the LS pattern with a line width of 18 nm and a pitch of 36 nm formed with the optimum exposure dose, by using a defect inspection apparatus KLA2360 (trade name) manufactured by KLA-Tencor and setting a pixel size of the defect inspection apparatus to 0.16 μm and a threshold thereof to 20, the number of defects (number/cm²) extracted from a difference caused by superimposition on a comparative image in units of pixels was detected, and the number of defects (number/cm²) per unit area was calculated. Thereafter, development defects were classified and extracted from all defects by defect review, and the number of development defects (number/cm²) per unit area was calculated. A sample is rated “A” for a count of less than 0.5, “B” for a count of 0.5 to less than 1.0, “C” for a count of 1.0 to less than 5.0, and “D” for a count of 5.0 or more. A smaller value indicates better performance.

TABLE 3

| | | | | Optimum | | | | | |
|---|---|---|---|---|---|---|---|---|---|
| | | Resist | PEB | exposure | | | | Collapse | Devel- |
| | | compo- | temp. | dose | EL | LWR | DOF | limit | opment |
| | | sition | (° C.) | (mJ/cm²) | (%) | (nm) | (nm) | (nm) | defects |
| Example | 3-1 | R-1 | 100 | 31 | 18 | 2.2 | 110 | 11.3 | A |
| | 3-2 | R-2 | 100 | 32 | 18 | 2.2 | 120 | 11.2 | A |
| | 3-3 | R-3 | 100 | 32 | 19 | 2.1 | 110 | 10.8 | A |
| | 3-4 | R-4 | 95 | 32 | 17 | 2.3 | 100 | 11.1 | A |
| | 3-5 | R-5 | 100 | 31 | 16 | 2.2 | 110 | 11.2 | A |
| | 3-6 | R-6 | 100 | 32 | 17 | 2.2 | 120 | 11.1 | A |
| | 3-7 | R-7 | 105 | 33 | 17 | 2.3 | 100 | 11.1 | A |
| | 3-8 | R-8 | 100 | 33 | 18 | 2.1 | 100 | 10.9 | A |
| | 3-9 | R-9 | 100 | 31 | 19 | 2.2 | 110 | 11.0 | A |
| | 3-10 | R-10 | 100 | 32 | 18 | 2.3 | 120 | 11.2 | A |
| | 3-11 | R-11 | 105 | 33 | 17 | 2.4 | 110 | 10.7 | A |
| | 3-12 | R-12 | 100 | 34 | 16 | 2.2 | 120 | 11.1 | A |
| | 3-13 | R-13 | 95 | 32 | 19 | 2.1 | 110 | 10.8 | A |
| | 3-14 | R-14 | 100 | 31 | 17 | 2.2 | 100 | 10.9 | A |
| | 3-15 | R-15 | 100 | 32 | 18 | 2.3 | 120 | 11.1 | A |
| | 3-16 | R-16 | 100 | 33 | 17 | 2.3 | 100 | 11.3 | A |
| | 3-17 | R-17 | 100 | 32 | 17 | 2.2 | 100 | 11.2 | A |
| | 3-18 | R-18 | 100 | 33 | 18 | 2.1 | 120 | 11.1 | A |
| | 3-19 | R-19 | 100 | 32 | 19 | 2.2 | 110 | 11.0 | A |
| | 3-20 | R-20 | 100 | 32 | 18 | 2.4 | 120 | 10.9 | A |
| | 3-21 | R-21 | 100 | 32 | 16 | 2.3 | 100 | 11.1 | A |
| | 3-22 | R-22 | 105 | 31 | 17 | 2.2 | 110 | 11.2 | A |
| | 3-23 | R-23 | 100 | 32 | 18 | 2.1 | 120 | 11.1 | A |
| | 3-24 | R-24 | 100 | 33 | 17 | 2.2 | 110 | 11.4 | A |
| | 3-25 | R-25 | 105 | 32 | 18 | 2.3 | 110 | 11.2 | A |
| | 3-26 | R-26 | 100 | 31 | 18 | 2.2 | 100 | 10.8 | A |
| | 3-27 | R-27 | 95 | 32 | 19 | 2.2 | 120 | 10.9 | A |
| | 3-28 | R-28 | 105 | 33 | 16 | 2.3 | 110 | 11.0 | A |
| | 3-29 | R-29 | 100 | 32 | 17 | 2.4 | 110 | 11.1 | A |
| | 3-30 | R-30 | 95 | 33 | 18 | 2.2 | 100 | 11.2 | A |

TABLE 4

| | | | | Optimum | | | | | |
|---|---|---|---|---|---|---|---|---|---|
| | | Resist | PEB | exposure | | | | Collapse | Devel- |
| | | compo- | temp. | dose | EL | LWR | DOF | limit | opment |
| | | sition | (° C.) | (mJ/cm²) | (%) | (nm) | (nm) | (nm) | defects |
| Comparative | 2-1 | CR-1 | 100 | 39 | 12 | 3.2 | 70 | 14.1 | C |
| Example | 2-2 | CR-2 | 100 | 36 | 14 | 2.8 | 90 | 12.2 | C |
| | 2-3 | CR-3 | 105 | 37 | 13 | 3.0 | 80 | 12.1 | B |
| | 2-4 | CR-4 | 100 | 36 | 15 | 2.8 | 70 | 12.5 | B |
| | 2-5 | CR-5 | 100 | 37 | 14 | 2.7 | 80 | 13.2 | B |
| | 2-6 | CR-6 | 100 | 38 | 13 | 2.8 | 80 | 13.1 | C |
| | 2-7 | CR-7 | 100 | 36 | 12 | 2.7 | 70 | 12.4 | B |
| | 2-8 | CR-8 | 100 | 37 | 13 | 2.7 | 80 | 12.5 | C |
| | 2-9 | CR-9 | 105 | 36 | 14 | 2.8 | 90 | 13.3 | C |
| | 2-10 | CR-10 | 100 | 37 | 13 | 2.9 | 80 | 13.1 | C |
| | 2-11 | CR-11 | 100 | 37 | 14 | 2.6 | 90 | 12.6 | B |
| | 2-12 | CR-12 | 100 | 36 | 13 | 2.7 | 90 | 13.2 | B |
| | 2-13 | CR-13 | 100 | 36 | 14 | 2.8 | 100 | 12.5 | C |
| | 2-14 | CR-14 | 95 | 38 | 13 | 2.7 | 90 | 12.7 | B |
| | 2-15 | CR-15 | 105 | 37 | 12 | 3.1 | 100 | 13.2 | C |
| | 2-16 | CR-16 | 95 | 36 | 13 | 2.8 | 100 | 13.1 | B |
| | 2-17 | CR-17 | 100 | 37 | 15 | 2.8 | 60 | 12.5 | C |
| | 2-18 | CR-18 | 100 | 38 | 14 | 2.7 | 90 | 12.7 | B |
| | 2-19 | CR-19 | 100 | 38 | 13 | 2.6 | 80 | 12.8 | C |
| | 2-20 | CR-20 | 100 | 37 | 12 | 2.7 | 70 | 13.0 | B |

From the results presented in Tables 3 and 4, it has been found that the chemically amplified resist composition containing the photoacid generator of the present invention has favorable sensitivity and is excellent in EL, LWR, and DOF. In addition, it has been confirmed that the chemically amplified resist composition has a small value of collapse limit and is resistant to pattern collapse even in fine pattern formation. Furthermore, it has been confirmed that development defects are also suppressed. Therefore, it has been indicated that the chemically amplified resist composition of the present invention is suitable as a material for EUV lithography.

### [5] EUV Lithography Evaluation (2)

### Examples 4-1 to 4-30 and Comparative Examples 3-1 to 3-20

Each of the chemically amplified resist compositions (R-1 to R-30 and CR-1 to CR-20) was spin-coated on a Si substrate formed with a silicon-containing spin-on hard mask SHB-A940 (silicon content: 43 wt %) manufactured by Shin-Etsu Chemical Co., Ltd. in a film thickness of 20 nm, and prebaked at 105° C. for 60 seconds using a hot plate to prepare a resist film having a film thickness of 50 nm. The resist film was exposed to light using an EUV scanner NXE3400 (NA: 0.33, a: 0.9/0.6, quadrupole illumination, mask of hole pattern having an on-wafer dimension of 46 nm as a pitch and +20% bias) manufactured by ASML, PEB was performed at temperatures described in Tables 5 and 6 for 60 seconds using a hot plate, and development was performed with a 2.38 wt % TMAH aqueous solution for 30 seconds to form a hole pattern having a dimension of 23 nm.

Using a length measuring SEM (CG6300) manufactured by Hitachi High-Tech Corporation, an exposure dose when a hole was formed with a dimension of 23 nm was measured and taken as sensitivity, the dimensions of 50 holes at this time were measured, and a value (3σ) three times a standard deviation (σ) calculated from the result was taken as CDU. Results thereof are presented in Tables 5 and 6.

TABLE 5

| | | | | Optimum | |
|---|---|---|---|---|---|
| | | Resist | PEB temp. | exposure dose | CDU |
| | | composition | (°C) | (mJ/cm²) | (nm) |
| Example | 4-1 | R-1 | 90 | 21 | 2.2 |
| | 4-2 | R-2 | 90 | 22 | 2.4 |
| | 4-3 | R-3 | 95 | 21 | 2.2 |
| | 4-4 | R-4 | 90 | 22 | 2.3 |
| | 4-5 | R-5 | 90 | 23 | 2.2 |
| | 4-6 | R-6 | 90 | 22 | 2.1 |
| | 4-7 | R-7 | 95 | 23 | 2.2 |
| | 4-8 | R-8 | 95 | 22 | 2.3 |
| | 4-9 | R-9 | 90 | 24 | 2.2 |
| | 4-10 | R-10 | 95 | 22 | 2.1 |
| | 4-11 | R-11 | 95 | 23 | 2.2 |
| | 4-12 | R-12 | 90 | 22 | 2.3 |
| | 4-13 | R-13 | 90 | 21 | 2.4 |
| | 4-14 | R-14 | 95 | 22 | 2.2 |
| | 4-15 | R-15 | 95 | 23 | 2.3 |
| | 4-16 | R-16 | 90 | 22 | 2.2 |
| | 4-17 | R-17 | 95 | 24 | 2.4 |
| | 4-18 | R-18 | 90 | 23 | 2.2 |
| | 4-19 | R-19 | 95 | 22 | 2.1 |
| | 4-20 | R-20 | 90 | 21 | 2.2 |
| | 4-21 | R-21 | 90 | 22 | 2.3 |
| | 4-22 | R-22 | 90 | 23 | 2.3 |
| | 4-23 | R-23 | 95 | 22 | 2.2 |
| | 4-24 | R-24 | 90 | 24 | 2.1 |
| | 4-25 | R-25 | 90 | 22 | 2.3 |
| | 4-26 | R-26 | 90 | 23 | 2.2 |
| | 4-27 | R-27 | 95 | 23 | 2.1 |
| | 4-28 | R-28 | 90 | 21 | 2.3 |
| | 4-29 | R-29 | 90 | 23 | 2.3 |
| | 4-30 | R-30 | 95 | 22 | 2.2 |

TABLE 6

| | | | | Optimum | |
|---|---|---|---|---|---|
| | | Resist | PEB temp. | exposure dose | CDU |
| | | composition | (°C) | (mJ/cm²) | (nm) |
| Comparative | 3-1 | CR-1 | 90 | 32 | 3.2 |
| Example | 3-2 | CR-2 | 90 | 28 | 2.7 |
| | 3-3 | CR-3 | 95 | 27 | 2.8 |
| | 3-4 | CR-4 | 90 | 26 | 2.7 |
| | 3-5 | CR-5 | 95 | 27 | 2.9 |
| | 3-6 | CR-6 | 90 | 28 | 2.8 |
| | 3-7 | CR-7 | 90 | 29 | 2.8 |
| | 3-8 | CR-8 | 90 | 28 | 2.7 |
| | 3-9 | CR-9 | 90 | 27 | 2.9 |
| | 3-10 | CR-10 | 95 | 27 | 2.8 |
| | 3-11 | CR-11 | 90 | 28 | 3.1 |
| | 3-12 | CR-12 | 90 | 26 | 2.8 |
| | 3-13 | CR-13 | 95 | 27 | 2.7 |
| | 3-14 | CR-14 | 90 | 28 | 2.8 |
| | 3-15 | CR-15 | 90 | 27 | 2.8 |
| | 3-16 | CR-16 | 90 | 27 | 2.7 |
| | 3-17 | CR-17 | 95 | 28 | 2.8 |
| | 3-18 | CR-18 | 90 | 27 | 2.9 |
| | 3-19 | CR-19 | 90 | 29 | 2.6 |
| | 3-20 | CR-20 | 90 | 27 | 2.7 |

From the results presented in Tables 5 and 6, it has been confirmed that the chemically amplified resist composition of the present invention has favorable sensitivity and is excellent in CDU.

Japanese Patent Application No. 2025-062666 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A sulfonium salt having the following formula (1): 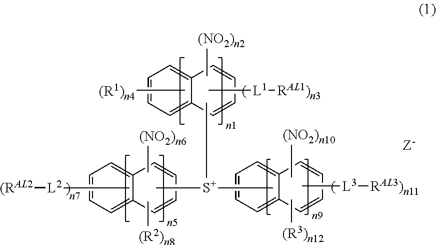
wherein n1 is 0 or 1, n2 is 0, 1, or 2, n3 is 0, 1, or 2, n4 is 0, 1, or 2, provided that 0≤n2+n3+n4≤5 when n1 is 0, and 0≤n2+n3+n4≤7 when n1 is 1, n5 is 0 or 1, n6 is 0, 1, or 2, n7 is 0, 1, or 2, n8 is 0, 1, or 2, provided that 0≤n6+n7+n8≤5 when n5 is 0, and 0≤n6+n7+n8≤7 when n5 is 1, n9 is 0 or 1, n10 is 0, 1, or 2, n11 is 0, 1, or 2, n12 is 0, 1, or 2, provided that 0≤n10+n11+n12≤5 when n9 is 0, and 0≤n10+n11+n12≤7 when n9 is 1, 1≤n2+n6+n10≤6, 1≤n3+n7+n11≤6,
R¹, R², and R³ are each independently a halogen atom, a cyano group, a hydroxy group, a carboxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n4 is 2, R¹s may be the same as or different from each other, and two R¹s may be bonded to each other to form a ring together with carbon atoms to which they are bonded, when n8 is 2, R²s may be the same as or different from each other, and two R²s may be bonded to each other to form a ring together with carbon atoms to which they are bonded, when n12 is 2, R³s may be the same as or different from each other, and two R³s may be bonded to each other to form a ring together with carbon atoms to which they are bonded,
L¹, L², and L³ are each independently an ether bond, an ester bond, or a carbonate bond, when n3 is 2, L¹s may be the same as or different from each other, when n7 is 2, L²s may be the same as or different from each other, when n11 is 2, L³s may be the same as or different from each other,
R^{AL1}, R^{AL2}, and R^{AL3} are each independently an acid-unstable group, when n3 is 2, R^{AL1}s may be the same as or different from each other, when n7 is 2, R^{AL2}s may be the same as or different from each other, when n11 is 2, R^{AL3}s may be the same as or different from each other,
two of the three aromatic rings bonded to S⁺ may be bonded to each other to form a ring together with the sulfur atom to which they are bonded, and
Z⁻ is an aromatic carboxylic acid anion which may have a substituent.

2. The sulfonium salt according to claim 1, having the following formula (1A): 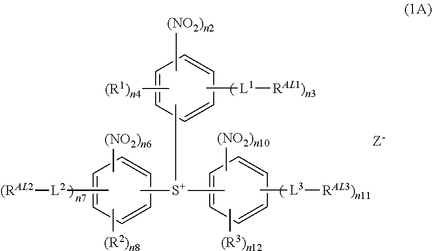
wherein n2 to n4, n6 to n8, n10 to n12, R¹ to R³, L¹ to L³, R^{AL1} to R^{AL3}, and Z⁻ are as defined above.

3. The sulfonium salt according to claim 1, wherein the acid-unstable group has the following formula (AL-1) or (AL-2): 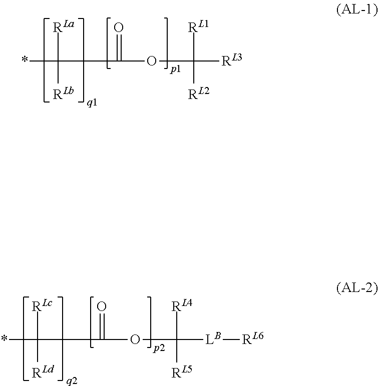
wherein p1 and p2 are each independently 0 or 1, q1 and q2 are each independently 0, 1, 2, 3, or 4,
R^{L1} and R^{L2} are each independently a C₁-C₁₂ hydrocarbyl group, R^{L3} is a hydrogen atom or a C₁-C₁₂ hydrocarbyl group, some of the (—CH₂-)s of the hydrocarbyl group of R^{L1}, R^{L2}, and R^{L3} may be replaced with —O— or —S—, when the hydrocarbyl group contains an aromatic ring, some or all of the hydrogen atoms of the aromatic ring may be replaced with a halogen atom, a cyano group, a nitro group, a C₁-C₄ alkyl group which may contain a halogen atom, or a C₁-C₄ alkoxy group which may contain a halogen atom, R^{L1} and R^{L2} may be bonded to each other to form a ring together with a carbon atom to which they are bonded, some of the (—CH₂-)s of the ring may be replaced with —O— or —S—, provided that when R^{L3} is a hydrogen atom, R^{L1} and R^{L2} are bonded to each other to form an alicyclic ring containing a multiple bond together with a carbon atom to which they are bonded, some of hydrogen atoms of the alicyclic ring may be replaced with a halogen atom,
R^{L4} and R^{L5} are each independently a hydrogen atom or a C₁-C₁₀ hydrocarbyl group, R^{L6} is a C₁-C₂₀ hydrocarbyl group, some of the (—CH₂-)s of the hydrocarbyl group may be replaced with —O— or —S—, R^{L5} and R^{L6} may be bonded to each other to form a C₃-C₂₀ heterocyclic group together with a carbon atom to which they are bonded and L⁴, some of the (—CH₂-)s of the heterocyclic group may be replaced with —O— or —S—,
L⁴ is —O— or —S—,
R^{La} to R^{Ld} are each independently a hydrogen atom or a C₁-C₂₀ hydrocarbyl group which may contain a heteroatom, and
* represents a bond with L¹, L², and L³.

4. The sulfonium salt according to claim 1, wherein Z⁻ is an anion having the following formula (Z): 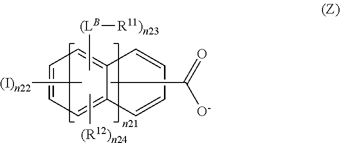
wherein n21 is 0 or 1, n22 is 0, 1, 2, 3, or 4, n23 is 0, 1, or 2, n24 is 0, 1, 2, 3, or 4, provided that 2≤n22+n23+n24≤5 when n21 is 0, provided that 2≤n22+n23+n24≤7 when n21 is 1,
L^{A} is a single bond, —O—, —S—, an ester bond, or a carbonate bond,
R¹¹ is a hydrogen atom or a C₁-C₂₀ hydrocarbyl group which may contain a heteroatom when L^{A} is a single bond, and is a hydrogen atom, a C₁-C₄₀ hydrocarbyl group which may contain a heteroatom (except for an acid-unstable group), or an acid-unstable group when L^{A} is —O—, —S—, an ester bond, or a carbonate bond,
R¹² is a halogen atom other than an iodine atom, a nitro group, a cyano group, a pentafluorosulfanyl group, a carboxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n24 is 2, 3, or 4, a plurality of R¹²s may be bonded to each other to form a ring structure together with carbon atoms on aromatic rings to which R¹²s are bonded, and some of the (—CH₂-)s of the rings may be replaced with —O— or —S—.

5. The sulfonium salt according to claim 4, wherein Z⁻ has the following formula (Z1): 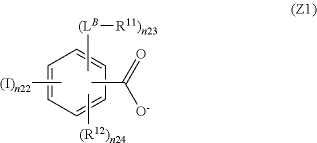
wherein n22 to n24, L^{A}, R¹¹, and R¹² are as defined above.

6. The sulfonium salt according to claim 4, wherein L^{A} is —O— or an ester bond.

7. The sulfonium salt according to claim 4, wherein L^{A} is —O—, —S—, an ester bond, or a carbonate bond, and R¹¹ is an acid-unstable group.

8. The sulfonium salt according to claim 4, wherein the acid-unstable group of R¹¹ has the following formula (AL-3) or (AL-4): 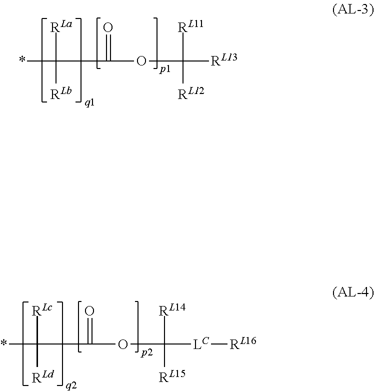
wherein p1 and p2 are each independently 0 or 1, q1 and q2 are each independently 0, 1, 2, 3, or 4,
R^{L11} and R^{L12} are each independently a C₁-C₁₂ hydrocarbyl group, R^{L13} is a hydrogen atom or a C₁-C₁₂ hydrocarbyl group, some of the (—CH₂-)s of the hydrocarbyl group of R^{L11}, R^{L12}, and R^{L13} may be replaced with —O— or —S—, when the hydrocarbyl group contains an aromatic ring, some or all of the hydrogen atoms of the aromatic ring may be replaced with a halogen atom, a cyano group, a nitro group, a C₁-C₄ alkyl group which may contain a halogen atom, or a C₁-C₄ alkoxy group which may contain a halogen atom, R^{L11} and R^{L12} may be bonded to each other to form a ring together with a carbon atom to which they are bonded, some of the (—CH₂-)s of the ring may be replaced with —O— or —S—, provided that when R^{L13} is a hydrogen atom, R^{L11} and R^{L12} are bonded to each other to form an alicyclic ring containing a multiple bond together with a carbon atom to which they are bonded, some of the hydrogen atoms of the alicyclic ring may be replaced with a halogen atom,
R^{L14} and R^{L15} are each independently a hydrogen atom or a C₁-C₁₀ hydrocarbyl group, R^{L16} is a C₁-C₂₀ hydrocarbyl group, some of the (—CH₂-)s of the hydrocarbyl group may be replaced with —O— or —S—, R^{L15} and R^{L16} may be bonded to each other to form a C₃-C₂₀ heterocyclic group together with a carbon atom to which they are bonded and L^{B}, some of the (—CH₂-)s of the heterocyclic group may be replaced with —O— or —S—,
L^{B} is —O— or —S—,
R^{La} to R^{Ld} are each independently a hydrogen atom or a C₁-C₂₀ hydrocarbyl group which may contain a heteroatom, and
* represents a bond with L^{A}.

9. A quencher formed of the sulfonium salt according to claim 1.

10. A chemically amplified resist composition comprising the quencher according to claim 9.

11. The chemically amplified resist composition according to claim 10, further comprising a base polymer containing a polymer containing at least one selected from a repeat unit having the following formula (a1), a repeat unit having the following formula (a2), and a repeat unit having the following formula (a3): 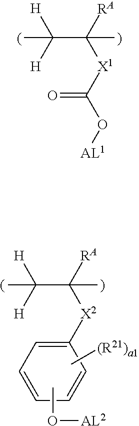
wherein R^{A}s are each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—N(H)—X¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
X² is a single bond, *—C(═O)—O—, or *—C(═O)—N(H)—X¹¹—,
* represents a bond with a carbon atom of a main chain,
R²¹ is a halogen atom, a cyano group, a hydroxy group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when a1 is 2, 3, or 4, R²¹s may be the same as or different from each other,
AL¹ and AL² are each independently an acid-unstable group, and
a1 is 0, 1, 2, 3, or 4, 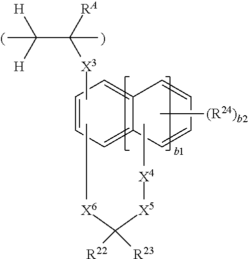
wherein b1 is 0 or 1, b2 is 0, 1, 2, or 3 when b1 is 0, and b2 is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * represents a bond with a carbon atom of a main chain,
X⁴ is a single bond, a C₁-C₄ aliphatic hydrocarbylene group, a carbonyl group, a sulfonyl group, or a group obtained by combining these groups,
X⁵ and X⁶ are each independently an oxygen atom or a sulfur atom, provided that X⁴ and X⁶ are bonded to adjacent aromatic ring carbon atoms,
R²² and R²³ are each independently a hydrogen atom or a C₁-C₂₀ hydrocarbyl group which may contain a heteroatom, R²² and R²³ may be bonded to each other to form a ring together with a carbon atom to which they are bonded,
R²⁴ is a halogen atom, a hydroxy group, a cyano group, a nitro group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}), R^{24A} and R^{24B} are each independently a hydrogen atom or a C₁-C₆ hydrocarbyl group, when b2 is 2 or more, R²⁴s may be the same as or different from each other, and the plurality of R²⁴s may be bonded to each other to form a ring together with aromatic ring carbon atoms to which they are bonded.

12. The chemically amplified resist composition according to claim 11, wherein the polymer contains a repeat unit having the following formula (b1) or (b2): 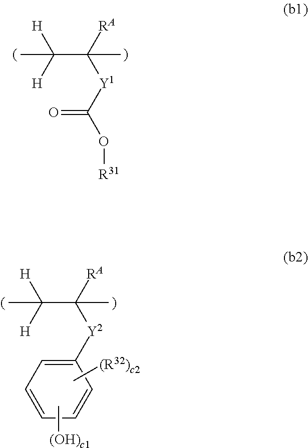
wherein R^{A}s are each independently a hydrogen atom, a fluorine atom, a methyl group, or a trifluoromethyl group,
Y¹ is a single bond, a phenylene group, a naphthylene group, *—C(═O)—O—Y¹¹—, or *—C(═O)—N(H)—Y¹¹—, the phenylene group or the naphthylene group may have a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom as a substituent, Y¹¹ is a C₁-C₁₀ saturated hydrocarbylene group, a phenylene group, or a naphthylene group, and the saturated hydrocarbylene group may contain a hydroxy group, an ether bond, an ester bond, or a lactone ring,
Y² is a single bond or *—C(═O)—O—,
* represents a bond with a carbon atom of a main chain,
R³¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—),
R³² is a halogen atom, a carboxy group, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when c2 is 2, 3, or 4, R³²s may be the same as or different from each other,
c1 is 1, 2, 3, or 4, and c2 is 0, 1, 2, 3, or 4, provided that 1≤c1+c2≤5.

13. The chemically amplified resist composition according to claim 11, wherein the polymer contains at least one selected from a repeat unit having the following formula (c1), a repeat unit having the following formula (c2), a repeat unit having the following formula (c3), a repeat unit having the following formula (c4), and a repeat unit having the following formula (c5): 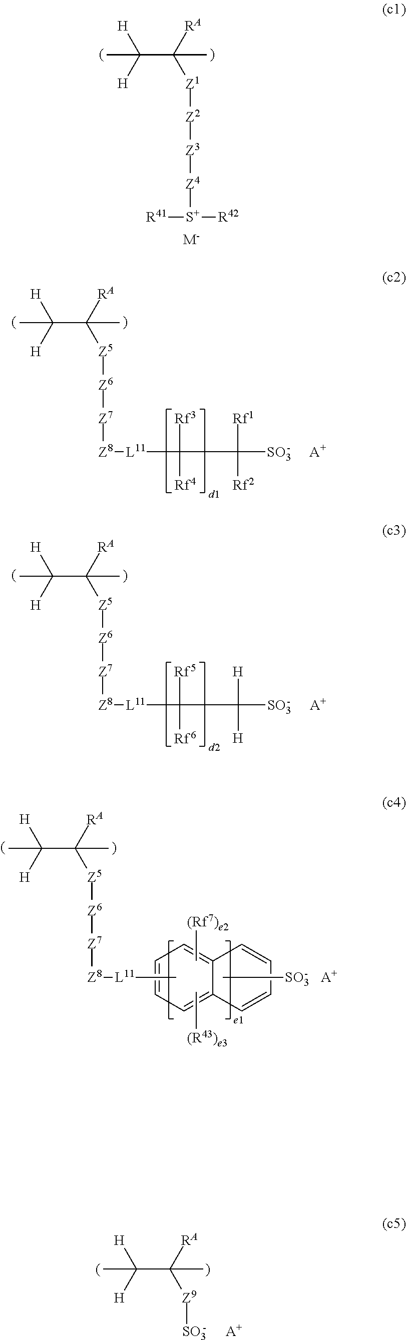
wherein d1 and d2 are each independently 0, 1, 2, or 3,
e1 is 0 or 1, e2 is 0, 1, 2, 3, or 4, e3 is 0, 1, 2, 3, or 4, provided that 0≤e2+e3≤4 when e1 is 0, and 0≤e2+e3≤6 when e1 is 1,
R^{A}s are each independently a hydrogen atom, a fluorine atom, a methyl group, or a trifluoromethyl group,
Z¹ is a single bond or a phenylene group which may have a substituent,
Z² is a single bond, **—C(═O)—O—Z²¹—, **—C(═O)—N(H)—Z²¹—, or **—O—Z²¹—, Z²¹ is a C₁-C₆ aliphatic hydrocarbylene group, a phenylene group, or a divalent group obtained by combining these groups, and may contain a halogen atom, a carbonyl group, an ester bond, an ether bond, or a hydroxy group,
Z³ is a single bond, an ether bond, an ester bond, a sulfonate bond, an amide bond, a sulfonamide bond, a carbonate bond, or a carbamate bond,
Z⁴ is a single bond, or a C₁-C₆ aliphatic hydrocarbylene group, a phenylene group, or a divalent group obtained by combining these groups, and may contain a halogen atom, a carbonyl group, an ester bond, an ether bond, or a hydroxy group,
Z⁵s are each independently a single bond, a phenylene group which may contain a substituent, a naphthylene group which may contain a substituent, *—C(═O)—O—Z⁵¹—, or *—C(═O)—N(R)—Z⁵¹—, Z⁵¹ is a C₁-C₁₀ aliphatic hydrocarbylene group, a phenylene group, or a naphthylene group, and the aliphatic hydrocarbylene group may contain a halogen atom, a hydroxy group, an ether bond, an ester bond, or a lactone ring, R is a hydrogen atom or a C₁-C₁₀ hydrocarbyl group which may contain a heteroatom,
Z⁶ is a single bond, an ether bond, an ester bond, a sulfonate bond, an amide bond, a sulfonamide bond, a carbonate bond, or a carbamate bond,
Z⁷s are each independently a single bond, ***—Z⁷¹—C(═O)—O—, ***—C(═O)—N(R)—Z⁷¹—, or ***—O—Z⁷¹—, Z⁷¹ is a C₁-C₂₀ hydrocarbylene group which may contain a heteroatom, R is as defined above,
Z⁸s are each independently a single bond, ****—Z⁸¹—C(═O)—O—, ****—C(═O)—N(R)—Z⁸¹—, or ****—O—Z⁸¹—, Z⁸¹ is a C₁-C₂₀ hydrocarbylene group which may contain a heteroatom, R is as defined above,
Z⁹ is a single bond, a methylene group, an ethylene group, a phenylene group, a fluorinated phenylene group, a phenylene group replaced with a trifluoromethyl group, *—C(═O)—O—Z⁹¹—, *—C(═O)—N(R)—Z⁹¹—, or *—O—Z⁹¹—, Z⁹¹ is a C₁-C₆ aliphatic hydrocarbylene group, a phenylene group, a fluorinated phenylene group, or a phenylene group replaced with a trifluoromethyl group, and may contain a carbonyl group, an ester bond, an ether bond, or a hydroxy group, R is as defined above,
* represents a bond with a carbon atom of a main chain, ** represents a bond with Z¹, *** represents a bond with Z⁶, **** represents a bond with Z⁷,
L¹¹ is a single bond, an ether bond, an ester bond, a carbonyl group, a sulfonate bond, a sulfonamide bond, a carbonate bond, or a carbamate bond,
Rf¹ and Rf² are each independently a fluorine atom or a C₁-C₆ fluorinated saturated hydrocarbyl group,
Rf³ and Rf⁴ are each independently a hydrogen atom, a fluorine atom, or a C₁-C₆ fluorinated saturated hydrocarbyl group,
Rf⁵ and Rf⁶ are each independently a hydrogen atom, a fluorine atom, or a C₁-C₆ fluorinated saturated hydrocarbyl group, provided that not all Rf⁵s and Rf⁶s are simultaneously hydrogen atoms,
Rf⁷ is a fluorine atom, a C₁-C₆ fluorinated alkyl group, a C₁-C₆ fluorinated alkoxy group, a C₁-C₆ fluorinated alkylthio group, or a pentafluorosulfanyl group, when e2 is 2, 3, or 4, Rf⁷s may be the same as or different from each other,
R⁴¹ and R⁴² are each independently a C₁-C₂₀ hydrocarbyl group which may contain a heteroatom, R⁴¹ and R⁴² may be bonded to each other to form a ring together with a sulfur atom to which they are bonded,
R⁴³ is a halogen atom other than a fluorine atom, or a C₁-C₂₀ hydrocarbyl group which may contain a heteroatom, when e3 is 2, 3, or 4, R⁴³s may be the same as or different from each other, and a plurality of R⁴³s may be bonded to each other to form a ring together with carbon atoms to which they are bonded,
M⁻ is a non-nucleophilic counter ion, and
A⁺ is an onium cation.

14. The chemically amplified resist composition according to claim 10, further comprising an organic solvent.

15. The chemically amplified resist composition according to claim 10, further comprising a photoacid generator that generates a strong acid.

16. The chemically amplified resist composition according to claim 10, further comprising a quencher other than the quencher.

17. The chemically amplified resist composition according to claim 10, further comprising a surfactant.

18. A pattern forming method comprising: a step of forming a resist film on a substrate using the chemically amplified resist composition according to claim 10; a step of exposing the resist film to a high energy ray; and a step of developing the exposed resist film using a developer.

19. The pattern forming method according to claim 18, wherein the high energy ray is a KrF excimer laser beam, an ArF excimer laser beam, an electron beam, or an extreme ultraviolet ray having a wavelength of 3 to 15 nm.
